(19) United States
(12) Patent Application Publication
(10) Pub. No.: US 2023/0200236 A1
(43) Pub. Date: Jun. 22, 2023

(54) LIGHT EMITTING DEVICE AND AMINE COMPOUND FOR LIGHT EMITTING DEVICE

(71) Applicant: Samsung Display Co., Ltd., Yongin-si, Gyeonggi-do (KR)

(72) Inventor: Takuya UNO, Yokohama (JP)

(21) Appl. No.: 17/935,894

(22) Filed: Sep. 27, 2022

(30) Foreign Application Priority Data
Dec. 21, 2021 (KR) 10-2021-0184025

(51) Int. Cl. H01L 51/00; C07D 307/91; C07D 409/12; C07D 333/76; C07D 409/14; C07D 405/12; C07D 405/14; H01L 51/50

(52) U.S. Cl. CPC H01L 51/0073; H01L 51/006; H01L 51/0074; H01L 51/0052; H01L 51/0059; H01L 51/0072; C07D 307/91; C07D 409/12; C07D 333/76; C07D 409/14; C07D 405/12; C07D 405/14; H01L 51/5036; C07B 2200/05

(57) ABSTRACT

A light emitting device that includes a first electrode, a second electrode facing the first electrode, and a plurality of functional layers between the first electrode and the second electrode is provided. At least one functional layer among the plurality of functional layers includes an amine compound represented by Formula 1:

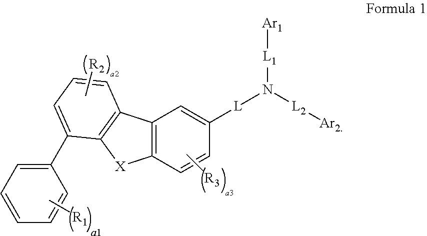

### CROSS-REFERENCE TO RELATED APPLICATION

This application claims priority to and the benefit of Korean Patent Application No. 10-2021-0184025, filed on Dec. 21, 2021, the entire content of which is hereby incorporated by reference.

### BACKGROUND

### 1. Field

Aspects of one or more embodiments of the present disclosure herein relate to an amine compound used in a light emitting device, and for example, to an amine compound used in a hole transport region and a light emitting device including the same.

### 2. Description of the Related Art

Recently, the development of an organic electroluminescence display apparatus as an image display apparatus is being actively conducted. Unlike liquid crystal display apparatuses and the like, the organic electroluminescence display apparatus is a self-luminescent display apparatus in which holes and electrons injected from a first electrode and a second electrode recombine in an emission layer, and thus a luminescent material including an organic compound in the emission layer emits light to implement display.

In the disclosure of an organic electroluminescence device to a display apparatus, there is a demand for an organic electroluminescence device having low driving voltage, high luminous efficiency, and a long service life, and development on materials for an organic electroluminescence device capable of stably attaining such characteristics is being continuously required (sought).

In addition, development on materials of a hole transport layer is being progressed in order to realize a highly efficient organic electroluminescence device.

### SUMMARY

Aspects of one or more embodiments of the present disclosure are directed to a light emitting device in which luminous efficiency and a device service life are improved (increased).

An embodiment of the present disclosure also provides an amine compound capable of improving luminous efficiency and a device service life of a light emitting device.

An embodiment of the present disclosure provides a light emitting device including a first electrode, a second electrode facing the first electrode, and a plurality of functional layers between the first electrode and the second electrode, wherein at least one functional layer among the plurality of functional layers includes an amine compound represented by Formula 1:

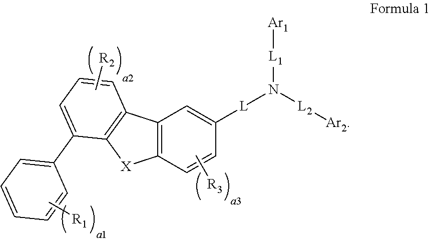

In Formula 1, X may be O or S, R₁ to R₃ may each independently be a hydrogen atom or a deuterium atom, a1 may be an integer from 0 to 5, a2 and a3 may each independently be an integer from 0 to 3, L may be a substituted or unsubstituted arylene group having 6 to 30 ring-forming carbon atoms, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms, Ar₁ may be represented by any one among Formula 2-1 to Formula 2-4, and Ar₂ may be a substituted or unsubstituted aryl group having 10 to 30 ring-forming carbon atoms, or a substituted or unsubstituted heteroaryl group having 2 to 30 ring-forming carbon atoms.

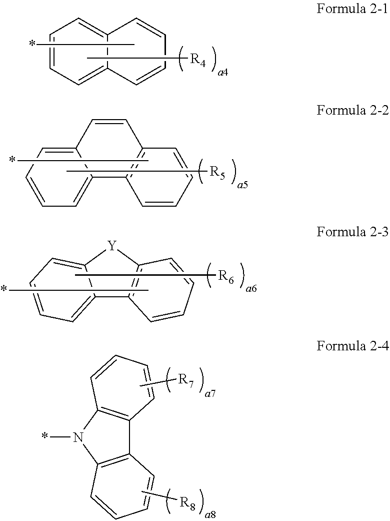

In Formula 2-1 and Formula 2-4, R₄ to R₈ may each independently be a hydrogen atom, a deuterium atom, a halogen atom, a substituted or unsubstituted alkyl group having 1 to 30 carbon atoms, a substituted or unsubstituted alkenyl group having 2 to 20 carbon atoms, a substituted or unsubstituted aryl group 6 to 30 ring-forming carbon atoms, or a substituted or unsubstituted heteroaryl group having 2 to 30 ring-forming carbon atoms, or are bonded to an adjacent group to form a ring, Y may be O, S, NR₉, or CR₁₀R₁₁, R₉ to R₁₁ may each independently be a substituted or unsubstituted alkyl group having 1 to 20 carbon atoms, or a substituted or unsubstituted aryl group 6 to 30 ring-forming carbon atoms, or are bonded to adjacent group to form a ring, a4 and a6 may each independently be an integer from 0 to 7, a5 may be an integer from 0 to 9, a7 and a8 may each independently be an integer from 0 to 4, and [symbol] may be a position linked to Formula 1.

In an embodiment, the plurality of functional layers may include a hole transport region on the first electrode, an emission layer on the hole transport region, and an electron transport region on the emission layer, and the hole transport region may include the amine compound represented by Formula 1.

In an embodiment, the hole transport region may include a hole injection layer on the first electrode, and the hole transport layer on the hole injection layer, and the hole transport layer may include the amine compound represented by Formula 1.

In an embodiment, the hole transport region may include a hole transport layer on the first electrode and an electron blocking layer on the hole transport layer, and the electron blocking layer may include the amine compound represented by Formula 1.

In an embodiment, Ar₂ may be represented by any one among Formula 3-1 to Formula 3-3 or any one among Formula 2-1 to Formula 2-4.

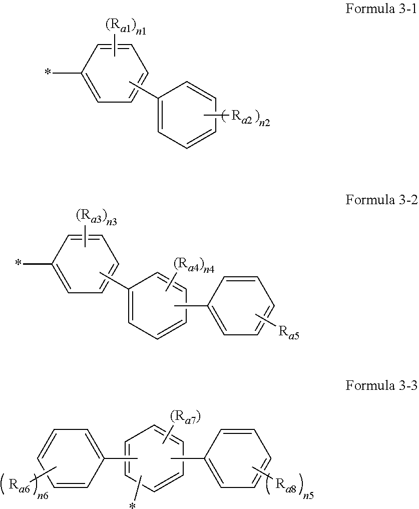

In Formula 3-1 to Formula 3-3, Rₐ₁ to Rₐ₈ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, n1, n3, and n4 may each independently be an integer from 0 to 4, n2, n5, n6, and n8 may each independently be an integer from 0 to 5, and n7 may be an integer from 0 to 3, and [symbol] may be a position linked to Formula 1.

In an embodiment, Ar₁ may be represented by Formula 2-1-1 or Formula 2-1-2:

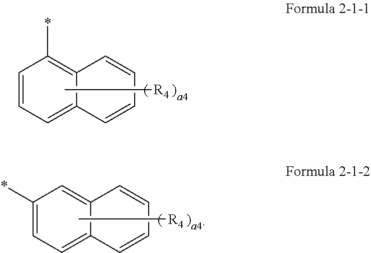

In Formula 2-1-1 and Formula 2-1-2, the same as defined in Formula 2-1 may be applied to R₄ and a4.

In an embodiment, Ar₁ may be represented by any one among Formula 2-2-1 to Formula 2-2-3:

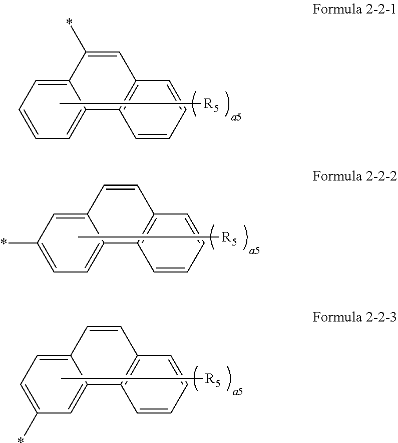

In Formula 2-2-1 to Formula 2-2-3, the same as defined in Formula 2-2 may be applied to R₅ and a5.

In an embodiment, Ar₁ may be represented by any one among Formula 2-3-1 to Formula 2-3-4:

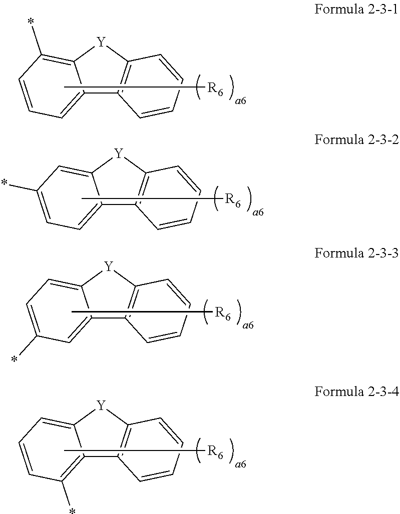

In Formula 2-3-1 to Formula 2-3-4, the same as defined in Formula 2-3 may be applied to Y, R₆ and a6.

In an embodiment, Ar₁ may be represented by any one among Formula 4-1 to Formula 4-7:

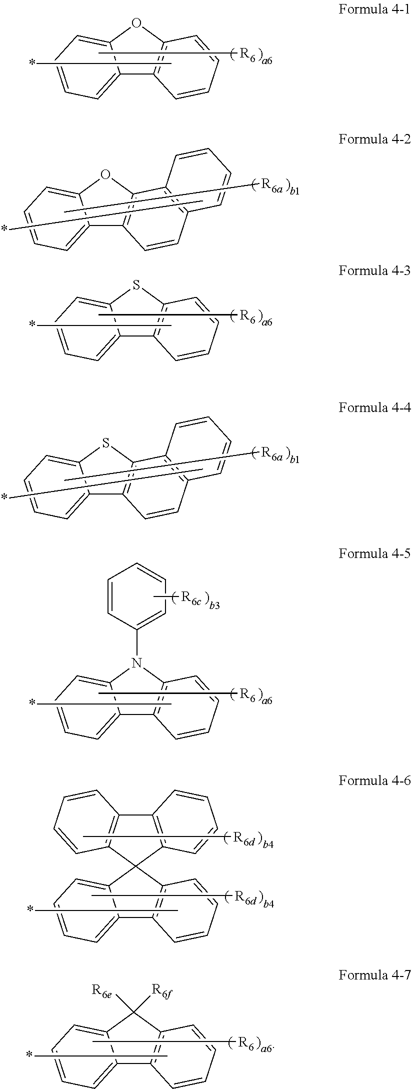

In Formula 4-1 to Formula 4-7, R₆ₐ to R_{6d} may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, R₆ₑ and R_{6f} may each independently be a substituted or unsubstituted alkyl group having 1 to 20 carbon atoms, or a substituted or unsubstituted aryl group having 6 to 30 ring-forming carbon atoms, b1 and b2 may each independently be an integer from 0 to 9, b3 may be an integer from 0 to 5, and b4 may be an integer from 0 to 8.

In Formula 4-1 to Formula 4-7, the same as defined in Formula 2-3 may be applied to R₆ and a6.

In an embodiment, the amine compound represented by Formula 1 may be represented by any one among Formula 5-1 to Formula 5-4:

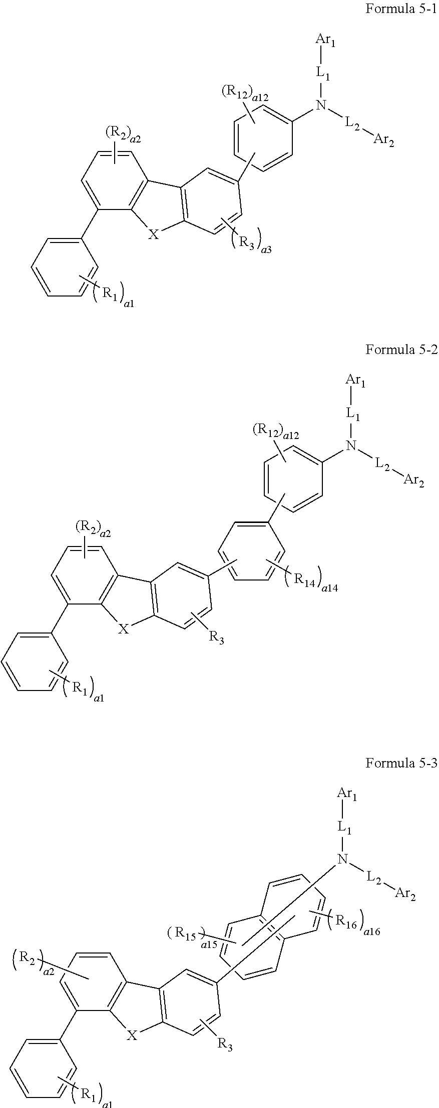

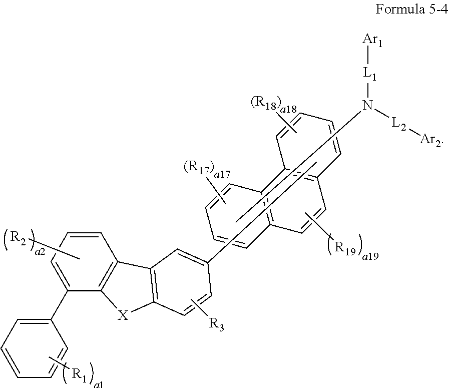

In Formula 5-1 to Formula 5-4, R₁₂ to R₁₉ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, a12 to a18 may each independently be an integer from 0 to 4, a19 may be an integer from 0 to 2, the sum of a15 and a16 may be 6 or less, and the sum of a17 to a19 may be 8 or less.

In Formula 5-1 to Formula 5-4, the same as defined in Formula 1 may be applied to R₁ to R₃, a1 to a3, L₁, L₂, X, Ar₁, and Ar₂.

In an embodiment, the amine compound represented by Formula 1 may be represented by any one among Formula 6-1 to Formula 6-3:

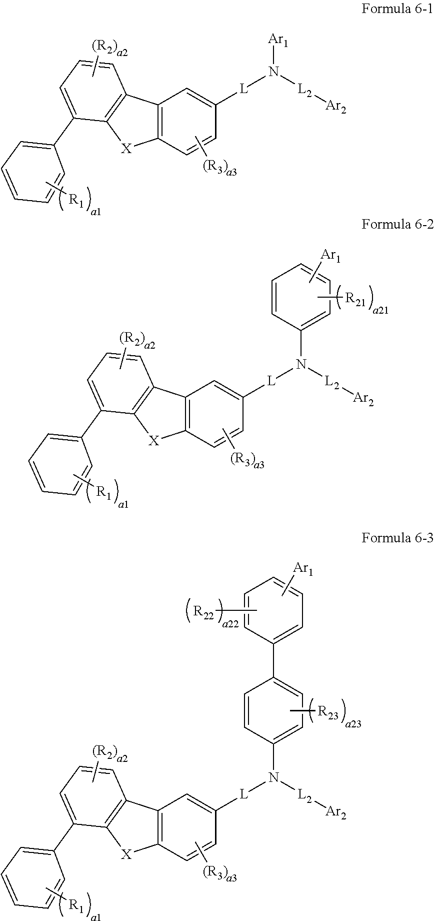

In Formula 6-1 to Formula 6-3, R₂₁ to R₂₃ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and a21 to a23 may each independently be an integer from 0 to 4.

In Formula 6-1 to Formula 6-3, the same as defined in Formula 1 may be applied to R₁ to R₃, a1 to a3, L, L₂, X, Ar₁, and Ar₂.

In an embodiment, L may be represented by any one among Formula L-1 to Formula L-7:

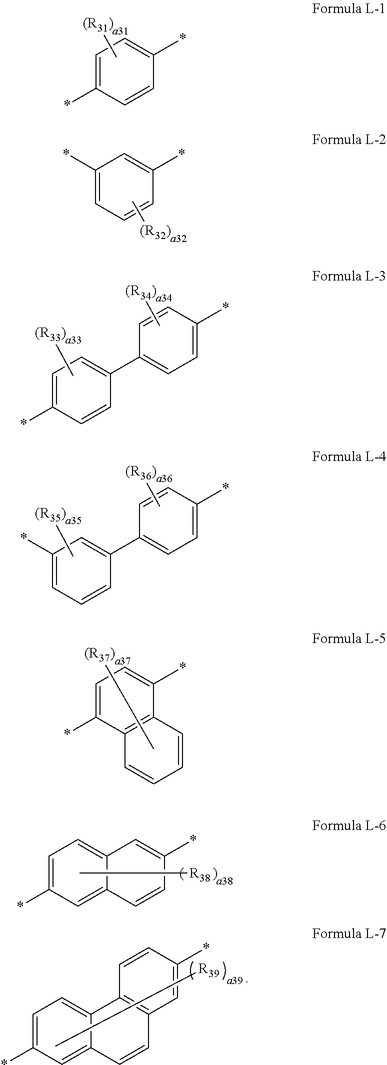

In Formula L-1 to Formula L-7, R₃₁ to R₃₉ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, a31 to a36 may each independently be an integer from 0 to 4, a37 and a38 may each independently be an integer from 0 to 6, and a39 may be an integer from 0 to 8.

In an embodiment of the present disclosure, an amine compound may be represented by Formula 1.

### BRIEF DESCRIPTION OF THE FIGURES

The accompanying drawings are included to provide a further understanding of the present disclosure, and are incorporated in and constitute a part of this disclosure. The drawings illustrate embodiments of the present disclosure and, together with the description, serve to explain principles of the present disclosure. In the drawings:

FIG. 1 is a plan view of a display apparatus according to an embodiment;

FIG. 2 is a cross-sectional view of a display apparatus according to an embodiment;

FIG. 3 is a cross-sectional view schematically illustrating a light emitting device according to an embodiment;

FIG. 4 is a cross-sectional view schematically illustrating a light emitting device according to an embodiment;

FIG. 5 is a cross-sectional view schematically illustrating a light emitting device according to an embodiment;

FIG. 6 is a cross-sectional view schematically illustrating a light emitting device according to an embodiment;

FIG. 7 is a cross-sectional view of a display apparatus according to an embodiment;

FIG. 8 is a cross-sectional view of a display apparatus according to an embodiment;

FIG. 9 is a cross-sectional view illustrating a display apparatus according to an embodiment; and

FIG. 10 is a cross-sectional view illustrating a display apparatus according to an embodiment.

### DETAILED DESCRIPTION

The present disclosure may be modified in many alternate forms, and thus specific embodiments will be exemplified in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numerals are used for referring to like elements, and duplicative descriptions thereof may not be provided. In the accompanying drawings, the dimensions of each structure may be exaggeratingly illustrated for clarity of the present disclosure. It will be understood that, although the terms “first”, “second”, etc. may be used herein to describe one or more suitable elements, these elements should not be limited by these terms. These terms are only used to distinguish one element from another. For example, a first element could be termed a second element, and, similarly, a second element could be termed a first element, without departing from the scope of the present disclosure. As used herein, the singular forms, “a,” “an,” and “the” are intended to include the plural forms as well, unless the context clearly indicates otherwise.

In the present disclosure, it will be understood that the terms “include,” “have” etc., specify the presence of a feature, a fixed number, a step, an operation, an element, a component, or a combination thereof disclosed in the disclosure, but do not exclude the possibility of presence or addition of one or more other features, fixed numbers, steps, operations, elements, components, or combination thereof.

In the present disclosure, when a part such as a layer, a film, a region, or a plate is referred to as being “on” or “above” another part, it can be directly on the other part, or an intervening part may also be present. In contrast, when a part such as a layer, a film, a region, or a plate is referred to as being “under” or “below” another part, it can be directly under the other part, or an intervening part may also be present. In addition, it will be understood that when a part is referred to as being “on” another part, it can be disposed on the other part, or disposed under the other part as well.

In the disclosure, the term “substituted or unsubstituted” may mean substituted or unsubstituted with at least one substituent selected from the group including (e.g., consisting of) a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents exemplified above may be substituted or unsubstituted. For example, a biphenyl group may be interpreted as an aryl group or a phenyl group substituted with a phenyl group.

In the disclosure, the phrase “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle.

The hydrocarbon ring and the heterocycle may be monocyclic or polycyclic. In addition, the rings formed by being bonded to each other may be connected to another ring to form a spiro structure.

In the disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In addition, two methyl groups in 4,5-dimethylphenanthrene may be interpreted as “adjacent groups” to each other.

In the disclosure, examples of the halogen atom may include a fluorine atom, a chlorine atom, a bromine atom, or an iodine atom.

In the disclosure, the alkyl group may be a linear, branched or cyclic type (kind). The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but the embodiment of the present disclosure is not limited thereto.

In the disclosure, an alkenyl group refers to a hydrocarbon group including at least one carbon double bond in the middle or terminal of an alkyl group having 2 or more carbon atoms. The alkenyl group may be a linear chain or a branched chain. The carbon number is not limited, but is 2 to 30, 2 to 20 or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the disclosure, an aryl group refers to any suitable functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

In the disclosure, the fluorenyl group may be substituted, and two substituents may be bonded to each other to form a spiro structure. Examples of embodiments in which the fluorenyl group is substituted are as follows. However, the embodiments of the present disclosure are not limited thereto.

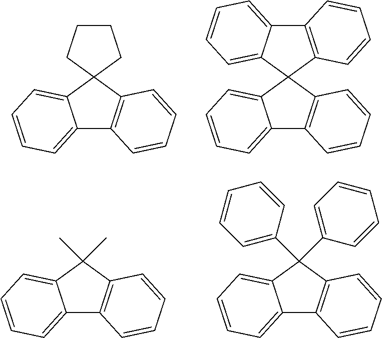

In the disclosure, the heteroaryl group may include at least one of B, O, N, P, Si, or S as a heteroatom. When the heteroaryl group contains two or more heteroatoms, the two or more heteroatoms may be the same as or different from each other. The heteroaryl group may be a monocyclic heteroaryl group or polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the disclosure, the above description of the aryl group may be applied to an arylene group except that the arylene group is a divalent group. The above description of the heteroaryl group may be applied to a heteroarylene group except that the heteroarylene group is a divalent group.

In the disclosure, the number of carbon atoms in an amine group is not limited, but may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, a triphenylamine group, etc., but the embodiment of the present disclosure is not limited thereto.

In the disclosure, a direct linkage may refer to a single bond.

In some embodiments “ [symbol] ” herein refers to a position to be connected.

Hereinafter, embodiments of the present disclosure will be described with reference to the accompanying drawings.

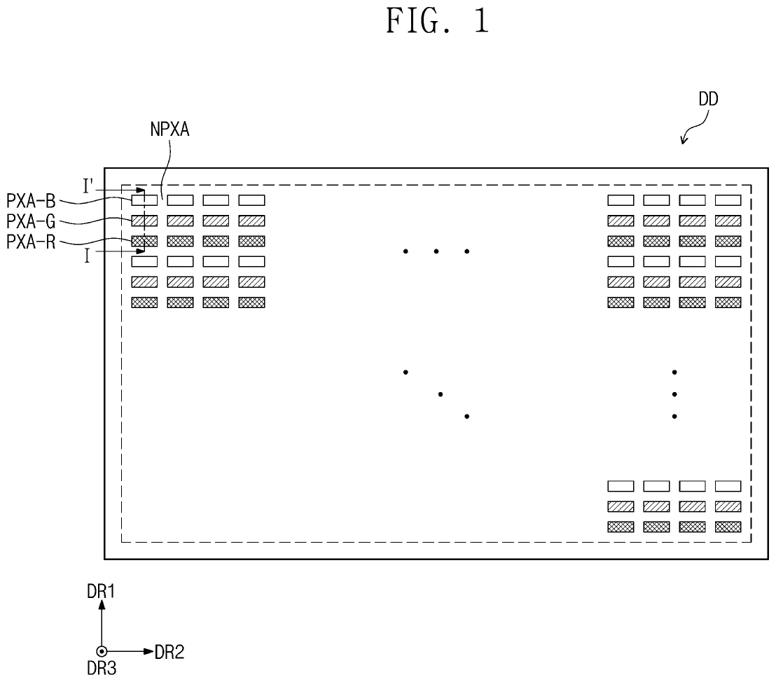

FIG. 1 is a plan view illustrating an embodiment of a display apparatus DD.

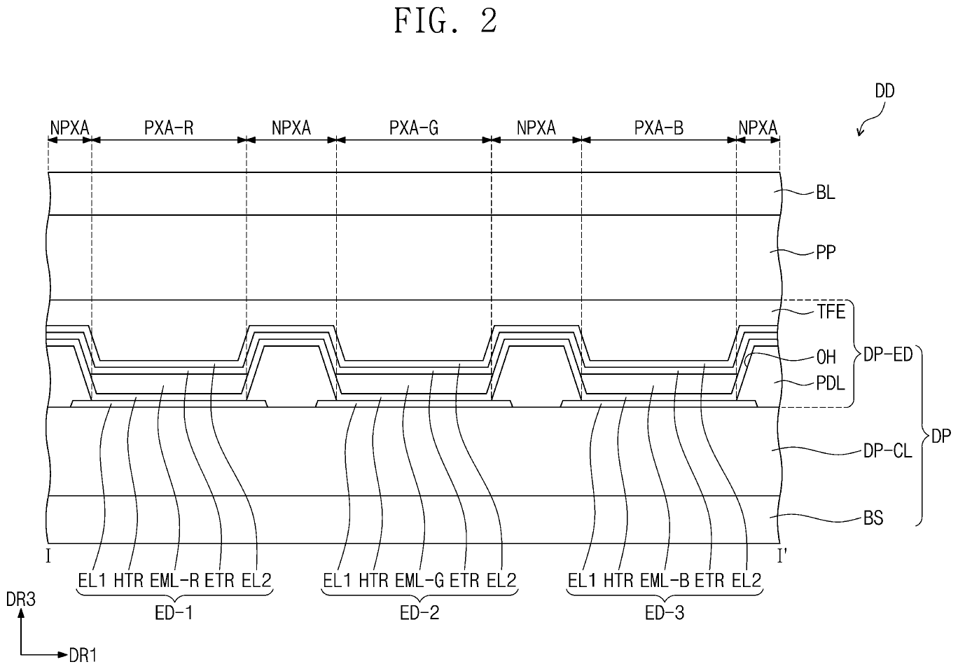

FIG. 2 is a cross-sectional view of the display apparatus DD of the embodiment. FIG. 2 is a cross-sectional view illustrating a part taken along line I-I′ of FIG. 1.

The display apparatus DD may include a display panel DP and an optical layer PP on the display panel DP. The display panel DP includes light emitting devices ED-1, ED-2, and ED-3. The display apparatus DD may include a plurality of light emitting devices ED-1, ED-2, and ED-3. The optical layer PP may be on the display panel DP and control reflected light in the display panel DP due to external light. The optical layer PP may include, for example, a polarization layer or a color filter layer. In some embodiments, the optical layer PP may not be provided from the display apparatus DD.

A base substrate BL may be on the optical layer PP. The base substrate BL may be a member which provides a base surface on which the optical layer PP is disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In addition, in an embodiment, the base substrate BL may not be provided.

The display apparatus DD according to an embodiment may further include a filling layer. The filling layer may be between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting devices ED-1, ED-2, and ED-3 between portions of the pixel defining film PDL, and an encapsulation layer TFE on the light emitting devices ED-1, ED-2, and ED-3.

The base layer BS may be a member which provides a base surface on which the display device layer DP-ED is disposed. The base layer BS may be a glass substrate, a metal substrate, a plastic substrate, etc. However, the embodiment is not limited thereto, and the base layer BS may be an inorganic layer, an organic layer, or a composite material layer.

In an embodiment, the circuit layer DP-CL is on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting devices ED-1, ED-2, and ED-3 of the display device layer DP-ED.

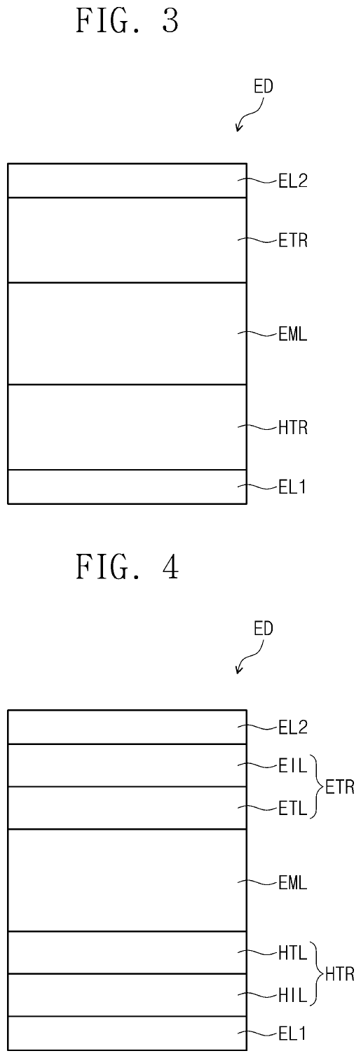

Each of the light emitting devices ED-1, ED-2, and ED-3 may have a structure of a light emitting device ED of an embodiment according to FIGS. 3 to 6, described below. Each of the light emitting devices ED-1, ED-2 and ED-3 may include a first electrode EL1, a hole transport region HTR, emission layers EML-R, EML-G and EML-B, an electron transport region ETR, and a second electrode EL2.

FIG. 2 illustrates an embodiment in which the emission layers EML-R, EML-G, and EML-B of the light emitting devices ED-1, ED-2, and ED-3 are in openings OH defined in the pixel defining film PDL, and the hole transport region HTR, the electron transport region ETR, and the second electrode EL2 are provided as a common layer in the entire light emitting devices ED-1, ED-2, and ED-3. However, the embodiment of the present disclosure is not limited thereto, and the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting devices ED-1, ED-2, and ED-3 in an embodiment may be provided by being patterned in an inkjet printing method.

The encapsulation layer TFE may cover the light emitting devices ED-1, ED-2 and ED-3. The encapsulation layer TFE may substantially seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display device layer DP-ED from moisture/oxygen, and the encapsulation-organic film protects (reduces the exposure to foreign materials) the display device layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not limited thereto. The encapsulation-organic film may include an acrylic-based compound, an epoxy-based compound, and/or the like. The encapsulation-organic film may include a photopolymerizable organic material, but the embodiment of the present disclosure is not limited thereto.

The encapsulation layer TFE may be on the second electrode EL2 and may be disposed filling the opening OH.

Referring to FIGS. 1 and 2, the display apparatus DD may include a non-light emitting region NPXA and light emitting regions PXA-R, PXA-G and PXA-B. The light emitting regions PXA-R, PXA-G and PXA-B may be regions in which light generated by the respective light emitting devices ED-1, ED-2 and ED-3 is emitted. The light emitting regions PXA-R, PXA-G, and PXA-B may be spaced apart from each other on a plane (e.g., in plan view).

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, which correspond to portions of the pixel defining film PDL. In some embodiments, in the disclosure, the light emitting regions PXA-R, PXA-G, and PXA-B may respectively correspond to pixels. The pixel defining film PDL may divide the light emitting devices ED-1, ED-2, and ED-3. The emission layers EML-R, EML-G and EML-B of the light emitting devices ED-1, ED-2 and ED-3 may be disposed in openings OH defined in the pixel defining film PDL and separated from each other.

The light emitting regions PXA-R, PXA-G and PXA-B may be divided into a plurality of groups according to the color of light generated from the light emitting devices ED-1, ED-2 and ED-3. In the display apparatus DD of an embodiment shown in FIGS. 1 and 2, three light emitting regions PXA-R, PXA-G, and PXA-B which emit red light, green light, and blue light, respectively are exemplarily illustrated. For example, the display apparatus DD of an embodiment may include the red light emitting region PXA-R, the green light emitting region PXA-G, and the blue light emitting region PXA-B that are separated from each other.

In the display apparatus DD according to an embodiment, the plurality of light emitting devices ED-1, ED-2 and ED-3 may emit light beams having wavelengths different from each other. For example, in an embodiment, the display apparatus DD may include a first light emitting device ED-1 that emits red light, a second light emitting device ED-2 that emits green light, and a third light emitting device ED-3 that emits blue light. For example, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, the embodiment of the present disclosure is not limited thereto, and the first to third light emitting devices ED-1, ED-2, and ED-3 may emit light beams in substantially the same wavelength range or at least one light emitting device may emit a light beam in a wavelength range different from the others. For example, the first to third light emitting devices ED-1, ED-2, and ED-3 may all emit blue light.

The light emitting regions PXA-R, PXA-G, and PXA-B in the display apparatus DD according to an embodiment may be arranged in a stripe form. Referring to FIG. 1, the plurality of red light emitting regions PXA-R may be arranged with each other along a second direction axis DR2, the plurality of green light emitting regions PXA-G may be arranged with each other along a second direction axis DR2, and the plurality of blue light emitting regions PXA-B each may be arranged with each other along the second direction axis DR2. In addition, the red light emitting region PXA-R, the green light emitting region PXA-G, and the blue light emitting region PXA-B may be alternately arranged in this order along a first direction axis DR1. (DR3 is a third direction which is normal or perpendicular to the plane defined by the first direction DR1 and the second direction DR2).

FIGS. 1 and 2 illustrate that all the light emitting regions PXA-R, PXA-G, and PXA-B have substantially similar areas, but the embodiment of the present disclosure is not limited thereto. Thus, the light emitting regions PXA-R, PXA-G, and PXA-B may have different areas from each other according to the wavelength range of the emitted light. In this embodiment, the areas of the light emitting regions PXA-R, PXA-G, and PXA-B may refer to areas when viewed on a plane defined by the first direction axis DR1 and the second direction axis DR2.

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display apparatus DD. For example, the arrangement form of the light emitting regions PXA-R, PXA-G, and PXA-B may be a PENTILE® arrangement form (PENTILE® arrangement form, for example, an RGBG matrix, an RGBG structure, or RGBG matrix structure) or a diamond arrangement form (Diamond Pixel™ arrangement form). PENTILE® is a duly registered trademark of Samsung Display Co., Ltd. Diamond Pixel™ is a trademark of Samsung Display Co., Ltd.

In some embodiments, the areas (i.e., sizes) of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

Hereinafter, FIGS. 3 to 6 are cross-sectional views schematically illustrating light emitting devices according to embodiments. Each of the light emitting devices ED according to embodiments may include a first electrode EL1, a hole transport region HTR, an emission layer EML, an electron transport region ETR, and a second electrode EL2 that are sequentially stacked.

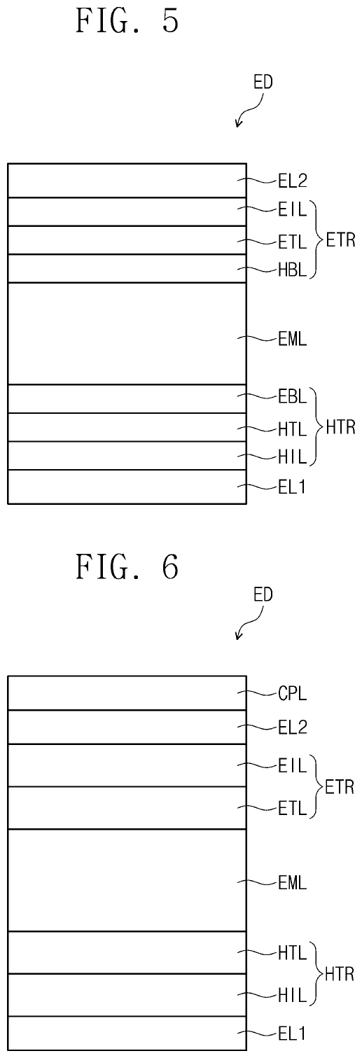

Compared with FIG. 3, FIG. 4 illustrates a cross-sectional view of a light emitting device ED of an embodiment, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light emitting device ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting device ED of an embodiment including a capping layer CPL on a second electrode EL2.

The first electrode EL1 has conductivity. The first electrode EL1 may be formed of a metal material, a metal alloy, and/or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, Zn, compounds comprising one or more of the foregoing elements, combinations of two or more of the foregoing elements or compounds, mixtures of two or more of the foregoing elements or compounds, and oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compounds or mixtures thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but the embodiment of the present disclosure is not limited thereto. However, an embodiment of the present disclosure is not limited thereto, and the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR may be provided on the first electrode EL1. The hole transport region HTR may include at least one of a hole injection layer HIL, a hole transport layer HTL, a buffer layer or an emission-auxiliary layer, or an electron blocking layer EBL. The thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but the embodiment of the present disclosure is not limited thereto.

The hole transport region HTR may be formed using one or more suitable methods such as a vacuum deposition method, a spin coating method, a cast method, a Langmuir-Blodgett (LB) method, an inkjet printing method, a laser printing method, and a laser induced thermal imaging (LITI) method.

The hole transport region HTR in the light emitting device ED of an embodiment may include an amine compound of an embodiment. The hole transport region HTR in the light emitting device ED of an embodiment may include an amine compound represented by Formula 1. The hole transport region HTR in the light emitting device ED of an embodiment may include at least one of the hole injection layer HIL, the hole transport layer HTL, or electron blocking layer EBL, and at least one of the hole injection layer HIL, the hole transport layer HTL, or the electron blocking layer EBL may include the amine compound represented by Formula 1 according to an embodiment. For example, the hole transport layer HTL in the light emitting device ED of an embodiment may include an amine compound represented by Formula 1.

The amine compound of an embodiment includes an amine group and includes, as a substituent, a dibenzoheterole group linked to the amine group. The dibenzoheterole group may be linked to the nitrogen atom of the amine group at the second carbon position, and a linker may be between the dibenzoheterole group and the nitrogen atom of the amine group. The dibenzoheterole group may be bonded to the amine group at the second carbon position with a linker located therebetween, and thus a highest occupied molecular orbital (HOMO) expands, thereby contributing to the improvement in stability of radical or radical cation state. In some embodiments, the dibenzoheterole ring is oriented so as to facilitate the intermolecular interaction, and thus a hole transport property may be improved.

The amine compound of an embodiment may include, as a substituent, a substituted or unsubstituted phenyl group at the sixth carbon position of the dibenzoheterole skeleton, thereby sterically protecting a heteroatom in the dibenzoheterole skeleton, and thus the stability of materials during the driving of the device may be improved. The linkage position between the dibenzoheterole group and the amine group works synergistically with the effect by the introduction of a specific (suitable) substituent of the dibenzoheterole group, and thus when the amine compound of an embodiment of the present disclosure is applied to a light emitting device, high efficiency and a long service life may be realized.

Moreover, the amine compound of an embodiment may improve electron resistance and exciton resistance of the material by the introduction of a first substituent as a substituent besides the dibenzoheterole group. In an embodiment, the first substituent may be a substituted or unsubstituted naphthyl, a substituted or unsubstituted phenanthrene group, a substituted or unsubstituted fluorenyl group, a substituted or unsubstituted dibenzofuran group, a substituted or unsubstituted dibenzothiophene group, or a substituted or unsubstituted carbazole group. The first substituent may be directly bonded to the nitrogen atom of the amine group or linked thereto via a linker. The amine compound of an embodiment may include at least one first substituent, thereby improving the electron and exciton resistance. Thus, the amine compound of an embodiment is applied to the light emitting device, and thereby high luminous efficiency and a long service life may be realized. In some embodiments, in the disclosure, the first substituent may refer to one substituent selected from among Formula 2-1 to Formula 2-4 which will be described later.

The numbers of carbons and heteroatom constituting the dibenzoheterole group are represented by Formula H:

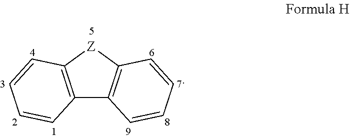

In Formula H, Z may be O or S. With respect to the carbon numbering of the dibenzoheterole group, in the embodiment in which the dibenzoheterole group is arranged such that Z is arranged on the top of the dibenzoheterole group like Formula H, the numbers are assigned in a clockwise direction from the carbon atom at the para position with Z from among the carbon atoms constituting the left benzene ring, and the carbon number at the condensation position is excluded.

The amine compound of an embodiment may be a monoamine compound. The amine compound may include one amine group in the compound structure.

The amine compound of an embodiment may be represented by Formula 1:

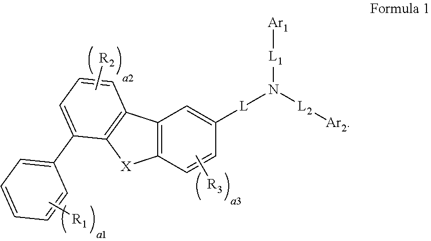

In Formula 1, X may be O or S. When X is O, the amine compound of an embodiment may include a dibenzofuran moiety. When X is S, the amine compound of an embodiment may include a dibenzothiophene moiety.

In Formula 1, R₁ to R₃ may each independently be a hydrogen atom or a deuterium atom. For example, R₁ and R₃ may each independently be a hydrogen atom.

In Formula 1, a1 may be an integer from 0 to 5. When a1 is 0, the amine compound of an embodiment may not be substituted with R₁. In Formula 1, the embodiment in which a1 is 5 and R₁s are all hydrogen atoms may be the same as the embodiment in which a1 is 0 in Formula 1. When a1 is an integer of 2 or more, a plurality of R₁s may all be the same, or at least one of the plurality of R₁s may be different from the others.

In Formula 1, a2 and a3 may each independently be an integer from 0 to 3. When each of a2 and a3 is 0, the amine compound of an embodiment may not be substituted with each of R₂ and R₃. The embodiment in which each of a2 and a3 is 3 and R₂s and R₃s are each hydrogen atoms may be the same as the embodiment in which each of a2 and a3 is 0. When each of a2 and a3 is an integer of 2 or more, a plurality of R₂s and R₃s may each be the same or at least one among the plurality of R₂s and R₃s may be different from the others.

In Formula 1, L may be a substituted or unsubstituted arylene group having 6 to 30 ring-forming carbon atoms. For example, L may be a substituted or unsubstituted phenylene group, a substituted or unsubstituted divalent biphenyl group, a substituted or unsubstituted divalent naphthyl group, or a substituted or unsubstituted divalent phenanthrene group.

In Formula 1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. For example, L₁ and L₂ may each independently be a direct linkage, a substituted or unsubstituted phenylene group, or a substituted or unsubstituted bivalent biphenyl group.

In Formula 1, Ar₁ may be represented by any one among Formula 2-1 to Formula 2-4. For example, the compound represented by Formula 1 of an embodiment may include the first substituent represented by any one among Formula 2-1 to Formula 2-4. The amine compound of an embodiment may include at least one first substituent. Accordingly, the amine compound of an embodiment may have an improvement in electron resistance and exciton resistance.

In Formula 1, Ar₂ may be a substituted or unsubstituted aryl group having 10 to 30 ring-forming carbon atoms, or a substituted or unsubstituted heteroaryl group having 2 to 30 ring-forming carbon atoms. For example, Ar₂ may be a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrene group, a substituted or unsubstituted fluorenyl group, a substituted or unsubstituted dibenzofuran group, a substituted or unsubstituted dibenzothiophene group, a substituted or unsubstituted benzo[b]naphtho[1,2-d]furan group, benzo[b]naphtho[2,1-d]thiophene group, or a substituted or unsubstituted carbazole group. In some embodiments, in Formula 1, Ar₂ may be represented by any one among Formula 2-1 to Formula 2-4:

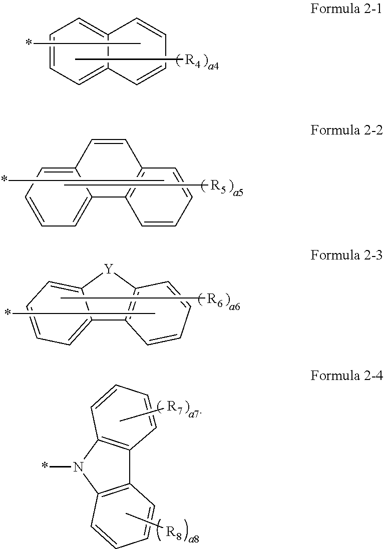

In Formula 2-1 to Formula 2-4, R₄ to R₈ may each independently be a hydrogen atom, a deuterium atom, a halogen atom, a substituted or unsubstituted alkyl group having 1 to 30 carbon atoms, a substituted or unsubstituted alkenyl group having 2 to 30 carbon atoms, a substituted or unsubstituted aryl group having 6 to 30 ring-forming carbon atoms, or a substituted or unsubstituted heteroaryl group having 2 to 30 ring-forming carbon atoms. Alternatively, each of R₄ to R₈ may be bonded to an adjacent group to form a ring. For example, R₄ to R₈ may each independently be a hydrogen atom or a substituted or unsubstituted phenyl group.

In Formula 2-3, Y may be O, S, NR₉, or CR₁₀R₁₁.

In Formula 2-3, R₉ to R₁₁ may each independently be a substituted or unsubstituted alkyl group having 1 to 20 carbon atoms, or a substituted or unsubstituted aryl group having 6 to 30 ring-forming carbon atoms. Alternatively, each of R₉ to R₁₁ may be bonded to an adjacent group to form a ring. For example, R₉ to R₁₁ may each independently be a substituted or unsubstituted methyl group, or a substituted or unsubstituted phenyl group.

In Formula 2-1 and Formula 2-3, a4 and a6 may each independently be an integer from 0 to 7. When each of a4 and a6 is 0, the amine compound of an embodiment may not be substituted with each of R₄ and R₆. The embodiment in which each of a4 and a6 is 7 and R₄s and R₆s are each hydrogen atoms may be the same as the embodiment in which each of a4 and a6 is 0. When each of a4 and a6 is an integer of 2 or more, a plurality of R₄s and R₆s may each be the same or at least one among the plurality of R₄s and R₆s may be different from the others.

In Formula 2-2, a5 may be an integer from 0 to 9. When a5 is 0, the amine compound of an embodiment may not be substituted with R₅. In Formula 1, the embodiment in which a5 is 9 and R₅s are all hydrogen atoms may be the same as the embodiment in which a5 is 0 in Formula 1. When a5 is an integer of 2 or more, a plurality of R₅s may all be the same, or at least one of the plurality of R₅s may be different from the others.

In Formula 2-4, a7 and a8 may each independently be an integer from 0 to 4. When each of a7 and a8 is 0, the amine compound of an embodiment may not be substituted with each of R₇ and R₈. The embodiment in which each of a7 and a8 is 4 and R₇s and R₈s are each hydrogen atoms may be the same as the embodiment in which each of a7 and a8 is 0. When each of a7 and a8 is an integer of 2 or more, a plurality of R₇s and R₈s may each be the same or at least one among the plurality of R₇s and R₈s may be different from the others.

In Formula 2-1 to Formula 2-4, [symbol] may be a position linked to Formula 1.

The amine compound of an embodiment may include a structure represented by Formula 1. The amine compound of an embodiment may have a structure in which the dibenzoheterole group is linked to the amine group at the second carbon position. For example, the amine compound of an embodiment may have a structure in which the dibenzoheterole group is linked to the nitrogen atom of the amine group via a linker. In some embodiments, the dibenzoheterole group includes a substituted or unsubstituted phenyl group linked to the sixth carbon position. Thus, the amine compound of an embodiment may be oriented such that the HOMO orbital expands to thus contribute to the improvement in stability of radical or radical cation state and facilitate the intermolecular interaction, and thus the hole transport property may be improved. In some embodiments, the amine compound of an embodiment includes the first substituent represented by any one among Formula 2-1 to Formula 2-4. Accordingly, the amine compound of an embodiment may have an improvement in electron resistance and exciton resistance. Thus, when the amine compound of an embodiment is used as a hole transport material of the light emitting device, the high efficiency and a long service life of the light emitting device may be realized.

In an embodiment, Ar₂ may be represented by any one among Formula 3-1 to Formula 3-3 or any one among Formula 2-1 to Formula 2-4:

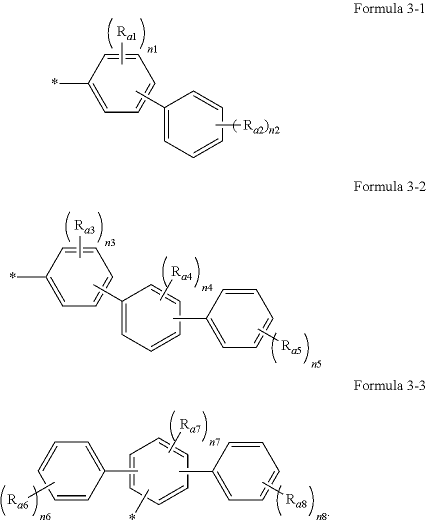

In Formula 3-1 to Formula 3-3, Rₐ₁ to Rₐ₈ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Alternatively, each of Rₐ₁ to Rₐ₈ may be bonded to an adjacent group to form a ring. For example, Rₐ₁ to Rₐ₈ may each independently be a hydrogen atom, a deuterium atom, or a substituted or unsubstituted phenyl group.

In Formula 3-1 and Formula 3-2, n1, n3, and n4 may each independently be an integer from 0 to 4. When each of n1, n3, and n4 is 0, the amine compound of an embodiment may not be substituted with each of Rₐ₁, Rₐ₃, and Rₐ₄. The embodiment in which each of n1, n3, and n4 is 4 and Rₐ₁s, Rₐ₃s and Rₐ₄s each are hydrogen atoms may be the same as the embodiment in which each of n1, n3, and n4 is 0. When each of n1, n3, and n4 is an integer of 2 or more, a plurality of Rₐ₁s, Rₐ₃s, and Rₐ₄s each may be the same or at least one among the plurality of Rₐ₁s, Rₐ₃s, and Rₐ₄s may be different from the others.

In Formula 3-1 to Formula 3-3, n2, n5, n6, and n8 may each independently be an integer from 0 to 5. When each of n2, n5, n6, and n8 is 0, the amine compound of an embodiment may not be substituted with each of Rₐ₂, Rₐ₅, Rₐ₆, and Rₐ₈. The embodiment in which each of n2, n5, n6, and n8 is 5 and Rₐ₂s, Rₐ₅s, Rₐ₆s and Rₐ₈s each are hydrogen atoms may be the same as the embodiment in which each of n2, n5, n6, and n8 is 0. When each of n2, n5, n6, and n8 is an integer of 2 or more, a plurality of Rₐ₂s, Rₐ₅s, Rₐ₆s and Rₐ₈s each may be the same or at least one among the plurality of Rₐ₂s, Rₐ₅s, Rₐ₆s and Rₐ₈s may be different from the others.

In Formula 3-3, n7 may be an integer from 0 to 3. In Formula 3-3, when n7 is 0, the amine compound of an embodiment may not be substituted with Rₐ₇. In Formula 3-3, the embodiment in which n7 is 3 and Rₐ₇s are all hydrogen atoms may be the same as the embodiment in which n7 is 0 in Formula 3-3. When n7 is an integer of 2 or more, a plurality of Rₐ₇s may all be the same, or at least one of the plurality of Rₐ₇s may be different from the others.

In Formula 3-1 to Formula 3-3, [symbol] may be a position linked to Formula 1.

In the amine compound represented by Formula 1 of an embodiment, Ar₁ and Ar₂ may each independently be represented by any one among Formula 2-1 to Formula 2-4. In this embodiment, the amine compound represented by Formula 1 of an embodiment may include two first substituents. For example, the amine compound of an embodiment may include two first substituents that are selected from among Formula 2-1 to Formula 2-4.

When each of Ar₁ and Ar₂ is represented by any one among Formula 2-1 to Formula 2-4, Ar₁ and Ar₂ may be the same as or different from each other. For example, both Ar₁ and Ar₂ may be represented by Formula 2-1, Formula 2-2, Formula 2-3, or Formula 2-4. Alternatively, Ar₁ may be represented by Formula 2-1, and Ar₂ may be represented by any one among Formulae 2-2 to 2-4, or Ar₁ may be represented by Formula 2-2, and Ar₂ may be represented by any one among Formulae 2-3 and 2-4, or Ar₁ may be represented by Formula 2-3, and Ar₂ may be represented by Formula 2-4. However, an embodiment of the present disclosure is not limited thereto.

In an embodiment, Ar₁ may be represented by Formula 2-1-1 or Formula 2-1-2. When Ar₁ is represented by Formula 2-1, Ar₁ may be represented by Formula 2-1-1 or Formula 2-1-2. When each of Ar₁ and Ar₂ is represented by Formula 2-1, Ar₁ and Ar₂ may each independently be represented by Formula 2-1-1 or Formula 2-1-2:

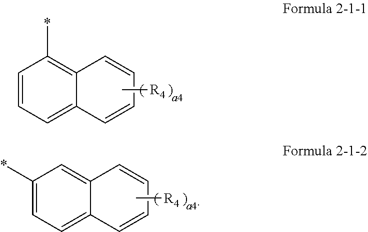

Formula 2-1-1 and Formula 2-1-2 represent the embodiments in which the position, at which the substituent represented by Formula 2-1 is linked to the amine group in Formula 1, is specified.

In Formula 2-1-1 and Formula 2-1-2, the same as described in Formula 2-1 may be applied to R₄ and a4.

In an embodiment, Ar₁ may be represented by any one among Formula 2-2-1 to Formula 2-2-3. When Ar₁ is represented by Formula 2-2, Ar₁ may be represented by any one among Formula 2-2-1 to Formula 2-2-3. In some embodiments, when each of Ar₁ and Ar₂ is represented by Formula 2-2, Ar₁ and Ar₂ may each independently be represented by any one among Formula 2-2-1 to Formula 2-2-3:

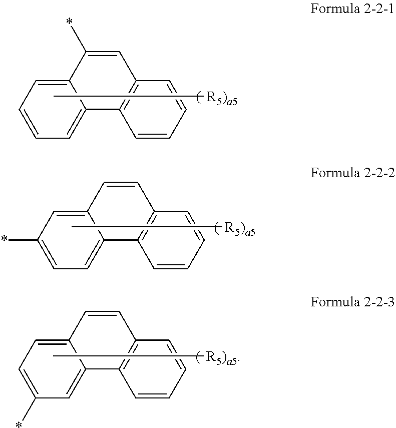

Formula 2-2-1 to Formula 2-2-3 represent the embodiments in which the position, at which the substituent represented by Formula 2-2 is linked to the amine group in Formula 1, is specified.

In Formula 2-2-1 to Formula 2-2-3, the same as described in Formula 2-2 may be applied to R₅ and a5.

In an embodiment, Ar₁ may be represented by any one among Formula 2-3-1 to Formula 2-3-4. When Ar₁ is represented by Formula 2-3, Ar₁ may be represented by any one among Formula 2-3-1 to Formula 2-3-4. In some embodiments, when each of Ar₁ and Ar₂ is represented by Formula 2-3, Ar₁ and Ar₂ may each independently be represented by any one among Formula 2-3-1 to Formula 2-3-4:

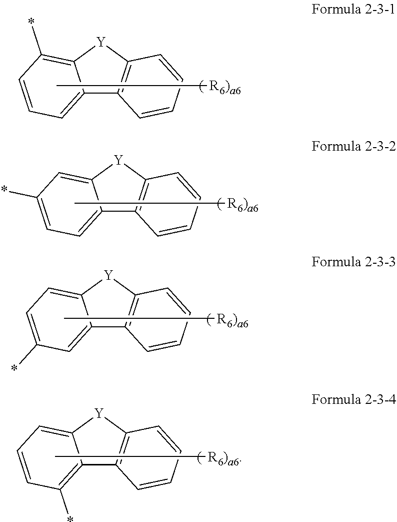

Formula 2-3-1 to Formula 2-3-4 represent the embodiments in which the position, at which the substituent represented by Formula 2-3 is linked to the amine group in Formula 1, is specified.

In Formula 2-3-1 to Formula 2-3-4, the same as described in Formula 2-3 may be applied to Y, R₆ and a6.

In an embodiment, Ar₁ may be represented by any one among Formula 4-1 to Formula 4-7. When Ar₁ is represented by Formula 2-3, Ar₁ may be represented by any one among Formula 4-1 to Formula 4-7. In some embodiments, when each of Ar₁ and Ar₂ is represented by Formula 2-3, Ar₁ and Ar₂ may each independently be represented by any one among Formula 4-1 to Formula 4-7:

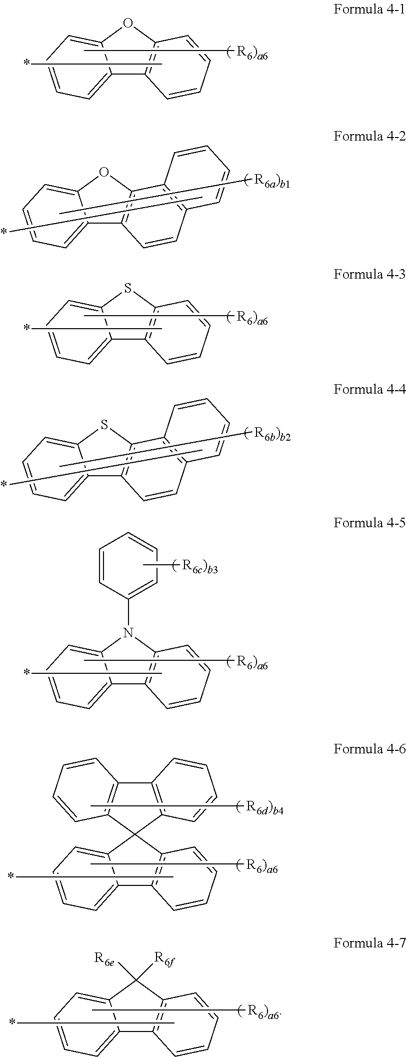

In Formula 4-1 to Formula 4-6, R₆ₐ to R_{6d} may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₆ₐ to R_{6d} may each independently be a hydrogen atom, or a substituted or unsubstituted phenyl group.

In Formula 4-7, R₆ₑ and R_{6f} may each independently be a substituted or unsubstituted alkyl group having 1 to 20 carbon atoms, or a substituted or unsubstituted aryl group having 6 to 30 ring-forming carbon atoms. For example, R₆ₑ and R_{6f} may each independently be a substituted or unsubstituted methyl group, or a substituted or unsubstituted phenyl group.

In Formula 4-2 and Formula 4-4, b1 and b2 may each independently be an integer from 0 to 9. When each of b1 and b2 is 0, the amine compound of an embodiment may not be substituted with each of R₆ₐ and R_{6b}. The embodiment in which each of b1 and b2 is 9 and R₆ₐs and R_{6b}s each are hydrogen atoms may be the same as the embodiment in which each of b1 and b2 is 0. When each of b1 and b2 is an integer of 2 or more, a plurality of R₆ₐs and R_{6b}s each may be the same or at least one among the plurality of R₆ₐs and R_{6b}s may be different from the others.

In Formula 4-5, b3 may be an integer from 0 to 5. In Formula 4-5, when b3 is 0, the amine compound of an embodiment may not be substituted with Rec. In Formula 4-5, the embodiment in which b3 is 5 and Recs are all hydrogen atoms may be the same as the embodiment in which b3 is 0 in Formula 4-5. When b3 is an integer of 2 or more, a plurality of R_{6c}s may all be the same, or at least one of the plurality of R_{6c}S may be different from the others.

In Formula 4-6, b4 may be an integer from 0 to 8. In Formula 4-6, when b4 is 0, the amine compound of an embodiment may not be substituted with R_{6d}. In Formula 4-6, the embodiment in which b4 is 8 and R_{6d}s are all hydrogen atoms may be the same as the embodiment in which b4 is 0 in Formula 4-6. When b4 is an integer of 2 or more, a plurality of R_{6d}s may be all the same or at least one of the plurality of R_{6d}s may be different from the others.

In Formula 4-1 to Formula 4-7, the same as described in Formula 2-3 may be applied to R₆ and a6.

In an embodiment, the amine compound represented by Formula 1 may be represented by any one among Formula 5-1 to Formula 5-4:

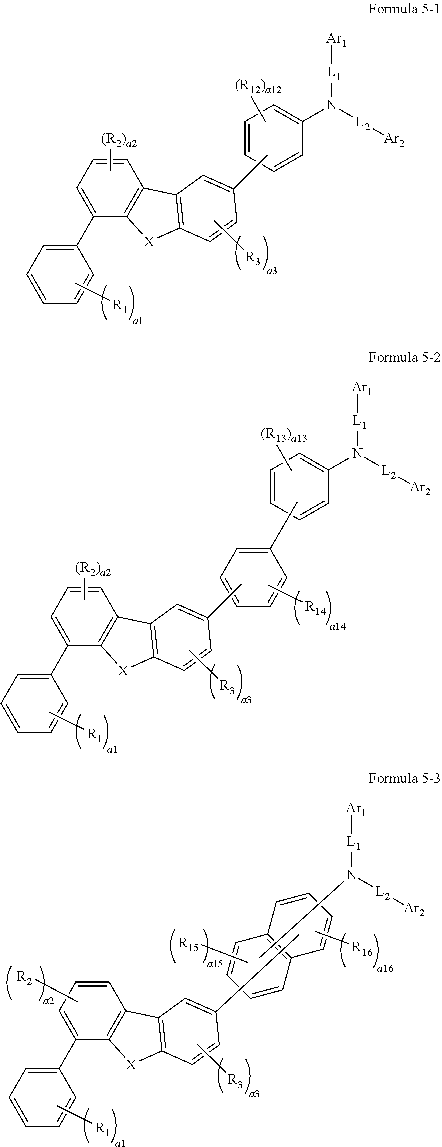

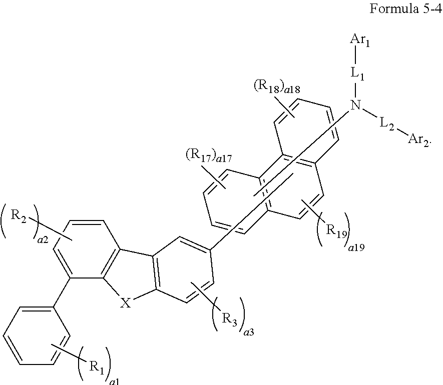

Formula 5-1 to Formula 5-4 represent the embodiments in which the types (kinds) of L in Formula 1 are specified. Formula 5-1 represents the embodiment in which L in Formula 1 is a substituted or unsubstituted phenylene group. Formula 5-2 represents the embodiment in which L in Formula 1 is a substituted or unsubstituted bivalent biphenyl group. Formula 5-3 represents the embodiment in which L in Formula 1 is a substituted or unsubstituted bivalent naphthyl group. Formula 5-4 represents the embodiment in which L in Formula 1 is a substituted or unsubstituted bivalent phenanthrene group.

In Formula 5-1 to Formula 5-4, R₁₂ to R₁₉ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₁₂ to R₁₉ may each independently be a hydrogen atom.

In Formula 5-1 to Formula 5-4, a12 to a18 may each independently be an integer from 0 to 4. When each of a12 to a18 is 0, the amine compound of an embodiment may not be substituted with each of R₁₂ to R₁₈. The embodiment in which each of a12 to a18 is 4 and R₁₂s to R₁₈s each are hydrogen atoms may be the same as the embodiment in which each of a12 to a18 is 0. When each of a12 to a18 is an integer of 2 or more, a plurality of R₁₂s to R₁₈s may each be the same or at least one among the plurality of R₁₂s to R₁₈s may be different from the others.

In Formula 5-4, a19 may be an integer from 0 to 2. In Formula 5-4, when a19 is 0, the amine compound of an embodiment may not be substituted with R₁₉. In Formula 5-4, the embodiment in which a19 is 2 and R₁₉s are all hydrogen atoms may be the same as the embodiment in which a19 is 0 in Formula 5-4. When a19 is an integer of 2 or more, a plurality of R₁₉s may all be the same, or at least one of the plurality of R₁₉s may be different from the others.

In an embodiment, the sum of a15 and a16 may be 6 or less, and the sum of a17 to a19 may be 8 or less.

In Formula 5-1 to Formula 5-4, the same as described in Formula 1 may be applied to R₁ to R₃, a1 to a3, L₁, L₂, X, Ar₁, and Ar₂.

In an embodiment, the amine compound represented by Formula 1 may be represented by any one among Formula 6-1 to Formula 6-3:

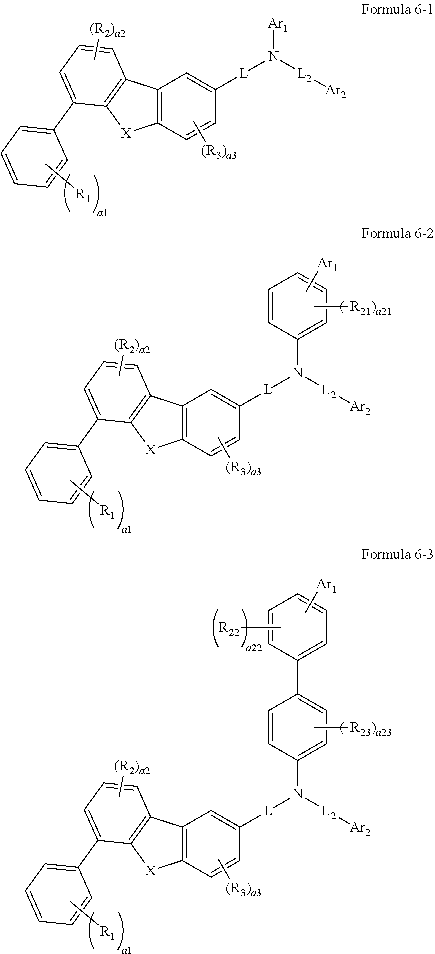

Formula 6-1 to Formula 6-3 represent the embodiments in which the types (kinds) of L₁ in Formula 1 are specified. Formula 6-1 represents the embodiment in which L₁ in Formula 1 is a direct linkage. Formula 6-2 represents the embodiment in which L₁ in Formula 1 is a substituted or unsubstituted phenylene group. Formula 6-3 represents the embodiment in which L₁ in Formula 1 is a substituted or unsubstituted bivalent biphenyl group.

In Formula 6-1 to Formula 6-3, R₂₁ to R₂₃ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₂₁ and R₂₃ may each independently be a hydrogen atom.

In Formula 6-2 and Formula 6-3, a21 to a23 may each independently be an integer from 0 to 4. When each of a21 to a23 is 0, the amine compound of an embodiment may not be substituted with each of R₂₁ to R₂₃. The embodiment in which each of a21 to a23 is 4 and R₂₁s to R₂₃s each are hydrogen atoms may be the same as the embodiment in which each of a21 to a23 is 0. When each of a21 to a23 is an integer of 2 or more, a plurality of R₂₁s to R₂₃s may each be the same or at least one among the plurality of R₂₁s to R₂₃s may be different from the others.

In Formula 6-1 to Formula 6-3, the same as described in Formula 1 may be applied to R₁ to R₃, a1 to a3, L, L₂, X, Ar₁, and Ar₂.

In the amine compound represented by Formula 1 of an embodiment, L may be represented by any one among Formula L-1 to Formula L-7:

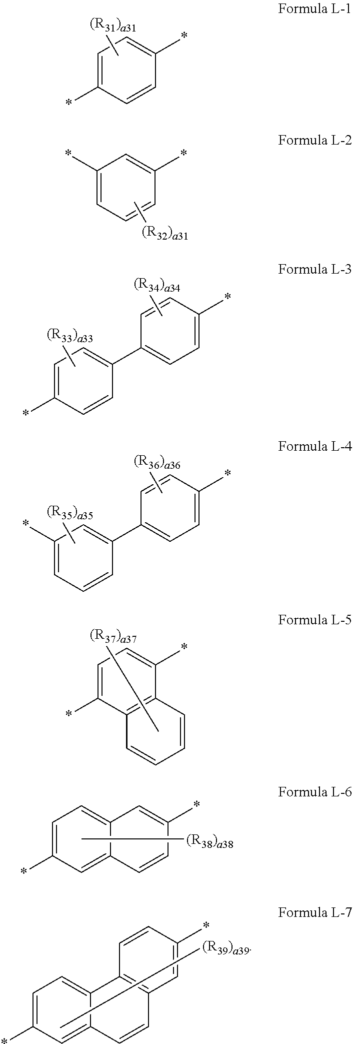

Formula L-1 to Formula L-7 are the embodiments in which the structure of L in Formula 1 is specified. For example, Formula L-1 to Formula L-7 are the embodiments in which the structure of a linker for linking the dibenzoheterole group and the nitrogen atom of the amine group is specified.

In Formula L-1 to Formula L-7, R₃₁ to R₃₉ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₃₁ to R₃₉ may each independently be a hydrogen atom.

In Formula L-1 to Formula L-4, a31 to a36 may each independently be an integer from 0 to 4. When each of a31 to a36 is 0, the amine compound of an embodiment may not be substituted with each of R₃₁ to R₃₆. The embodiment in which each of a31 to a36 is 4 and R₃₁s to R₃₆s each are hydrogen atoms may be the same as the embodiment in which each of a31 to a36 is 0. When each of a31 to a36 is an integer of 2 or more, a plurality of R₃₁s to R₃₆s may each be the same or at least one among the plurality of R₃₁s to R₃₆s may be different from the others.

In Formula L-5 and Formula L-6, a37 and a38 may each independently be an integer from 0 to 6. When each of a37 and a38 is 0, the amine compound of an embodiment may not be substituted with each of R₃₇ and R₃₈. The embodiment in which each of a37 and a38 is 6 and R₃₇s and R₃₈s are each hydrogen atoms may be the same as the embodiment in which each of a37 and a38 is 0. When each of a37 and a38 is an integer of 2 or more, a plurality of R₃₇s and R₃₈s may each be the same or at least one among the plurality of R₃₇s and R₃₈s may be different from the others.

In Formula L-7, a39 is an integer from 0 to 8. In Formula L-7, when a39 is 0, the amine compound of an embodiment may not be substituted with R₃₉. In Formula L-7, the embodiment in which a39 is 8 and R₃₉s are all hydrogen atoms may be the same as the embodiment in which a39 is 0 in Formula L-7. When a39 is an integer of 2 or more, a plurality of R₃₉s may all be the same, or at least one of the plurality of R₃₉s may be different from the others.

The amine compound may be any one of the compounds represented by Compound Group 1. The light emitting device ED of an embodiment may include at least one amine compound among the compounds represented by Compound Group 1 in the hole transport region HTR. In some embodiments, D in Compound Group 1 is a deuterium atom.

### Compound Group 1

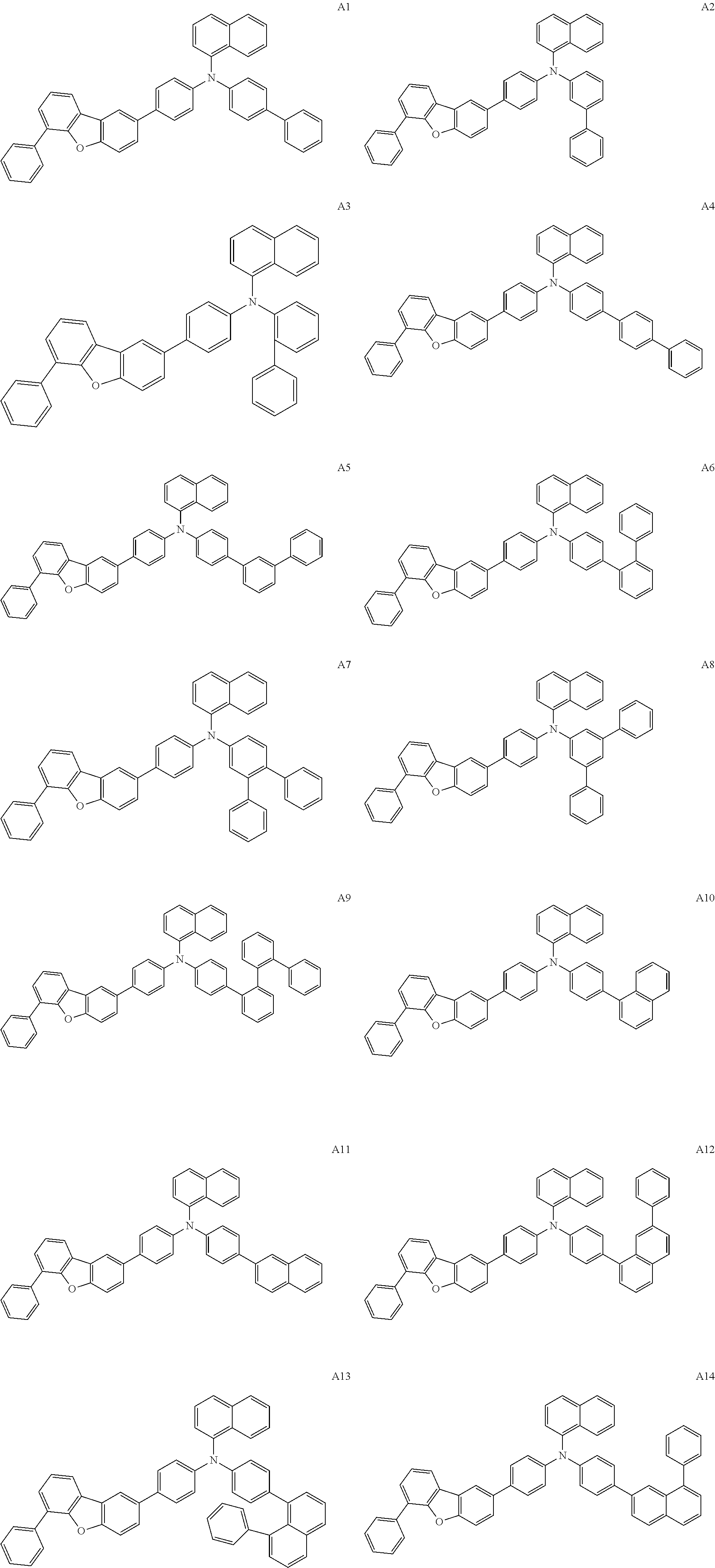

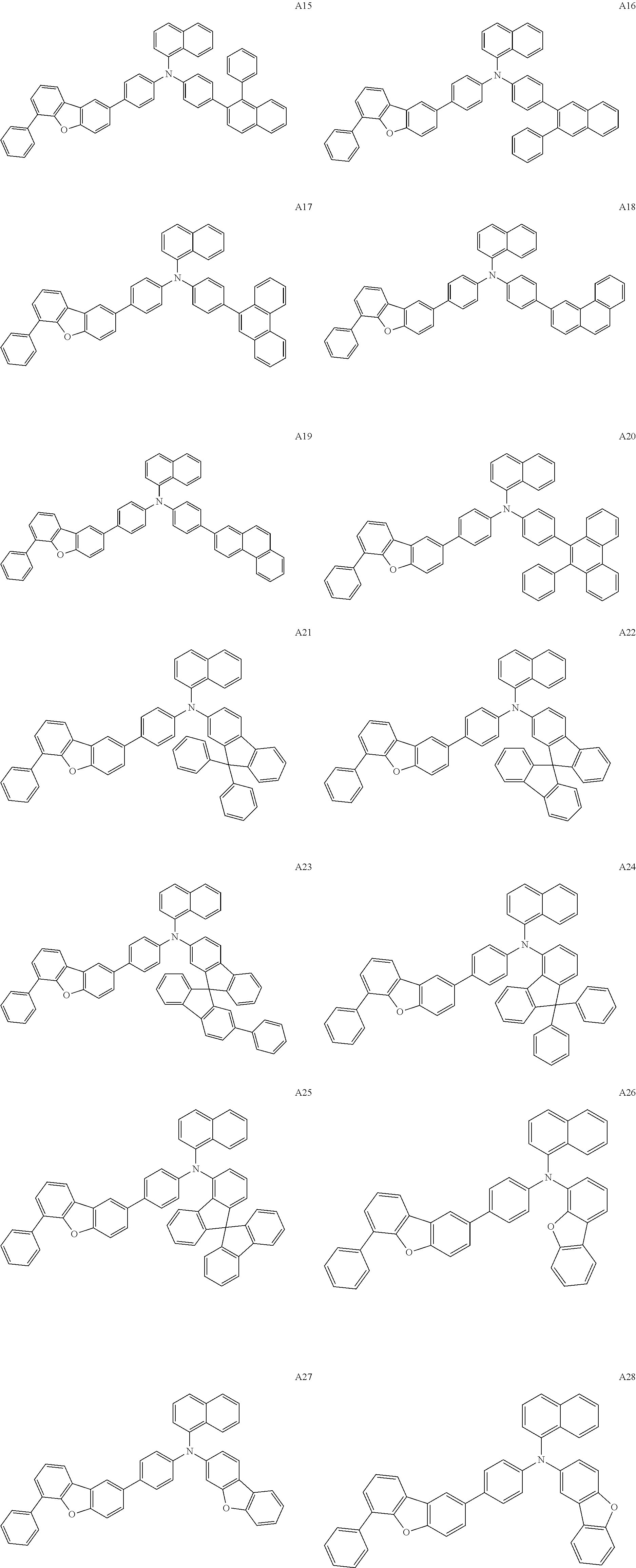

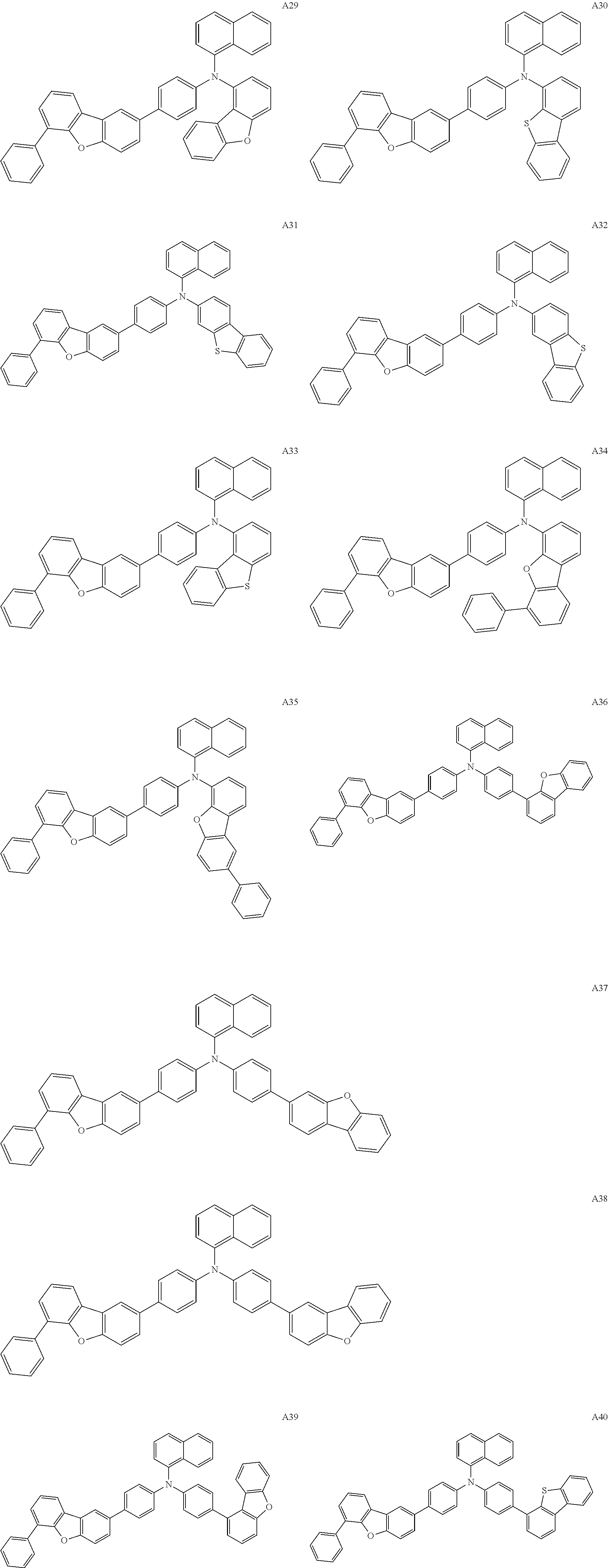

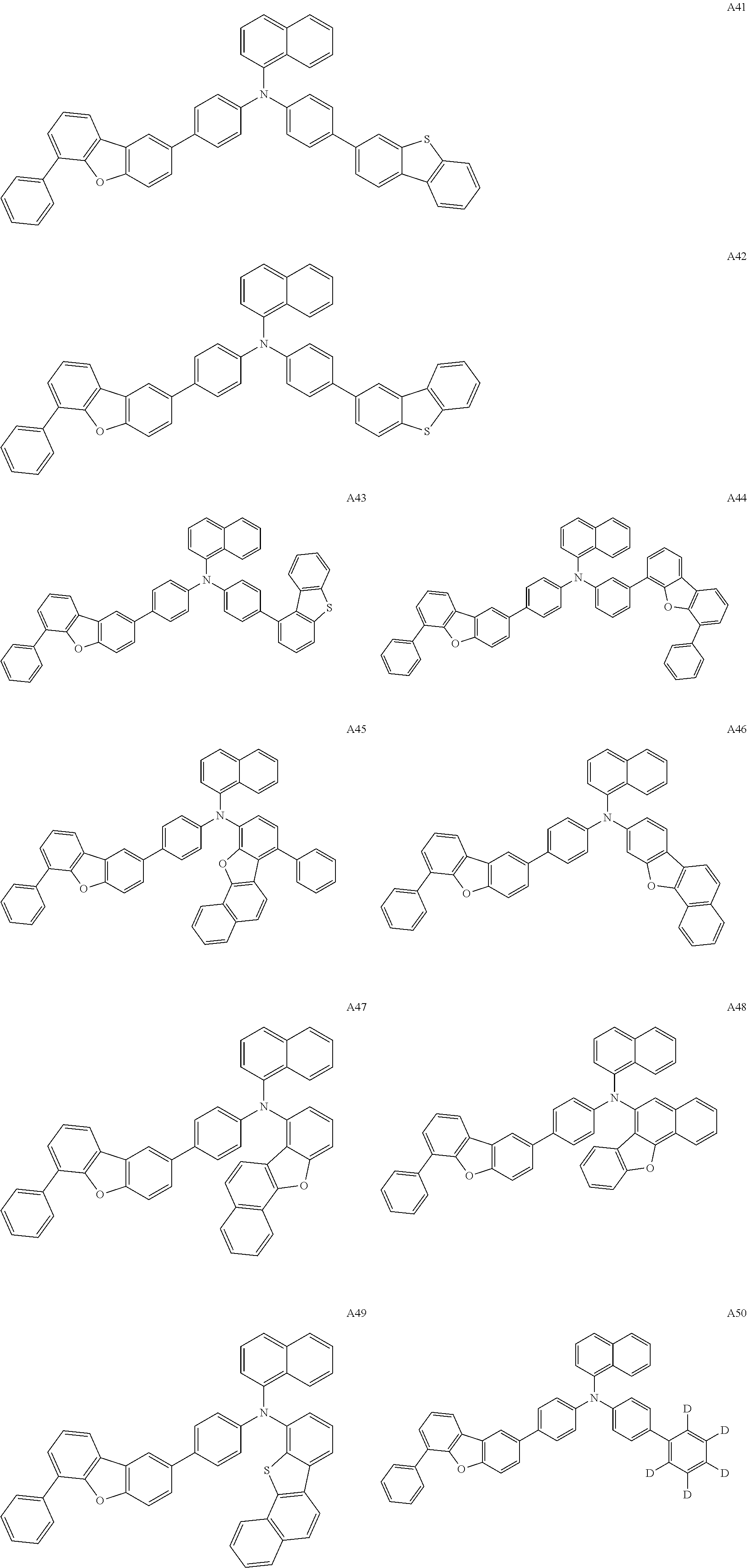

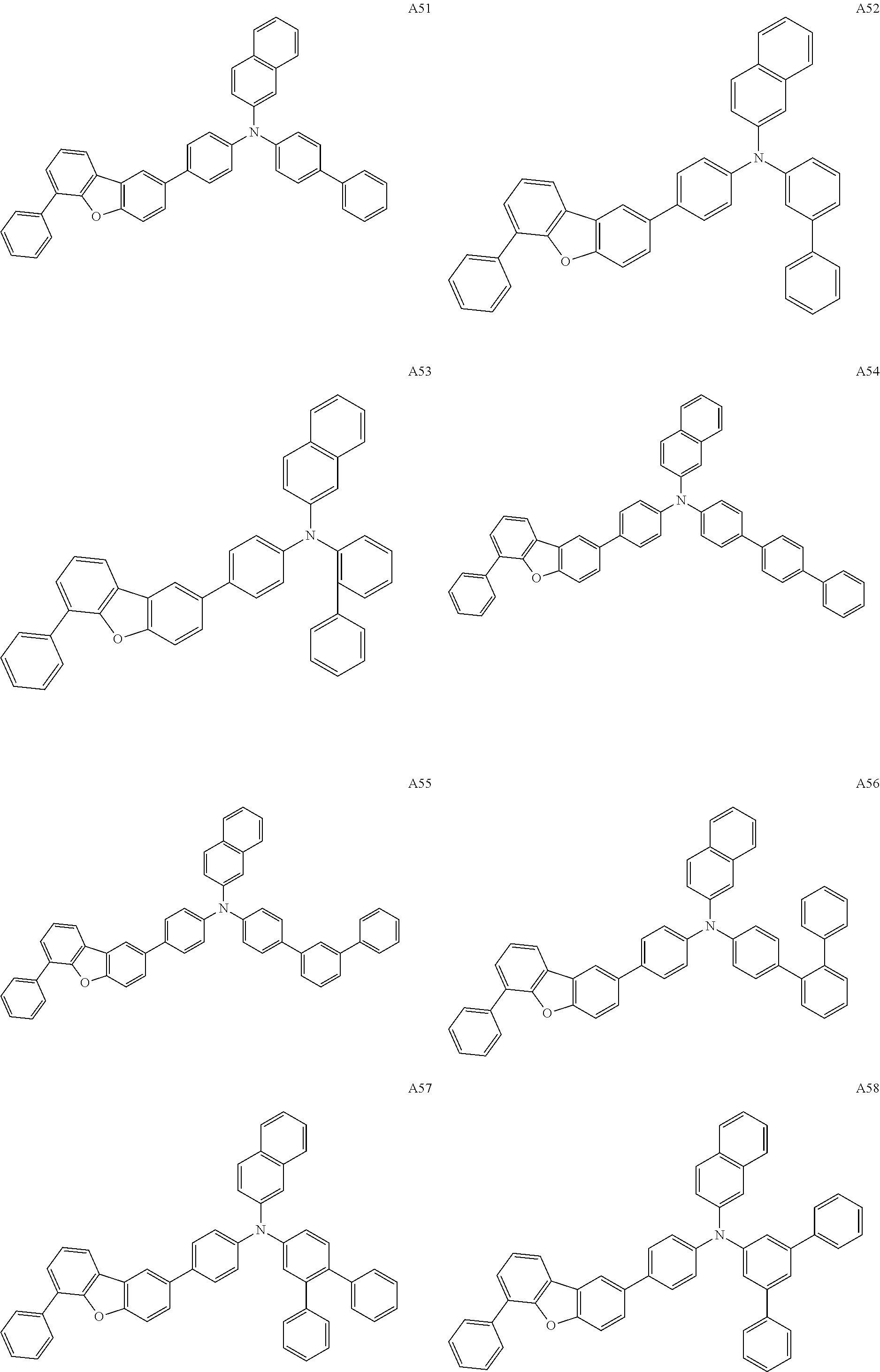

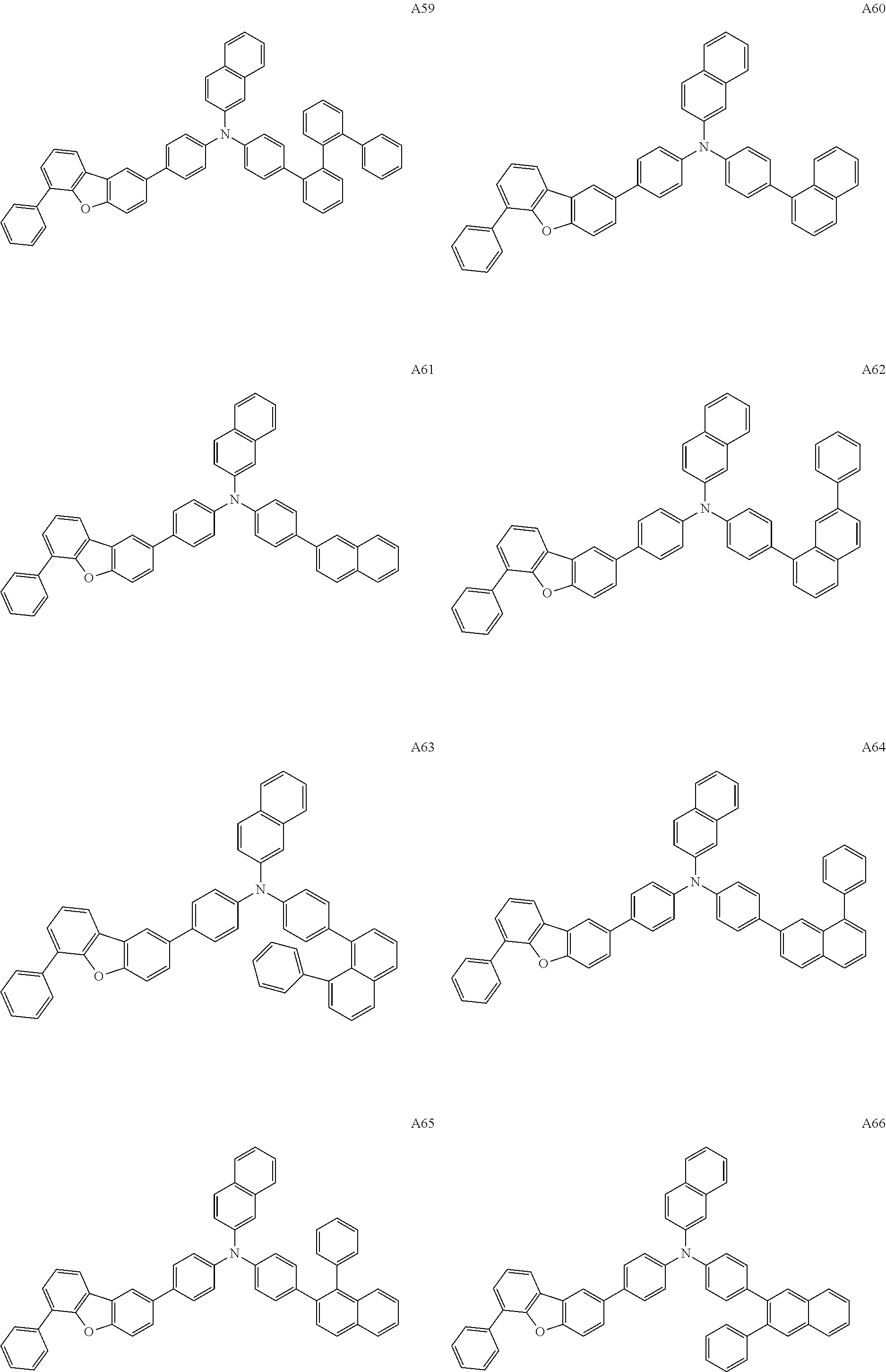

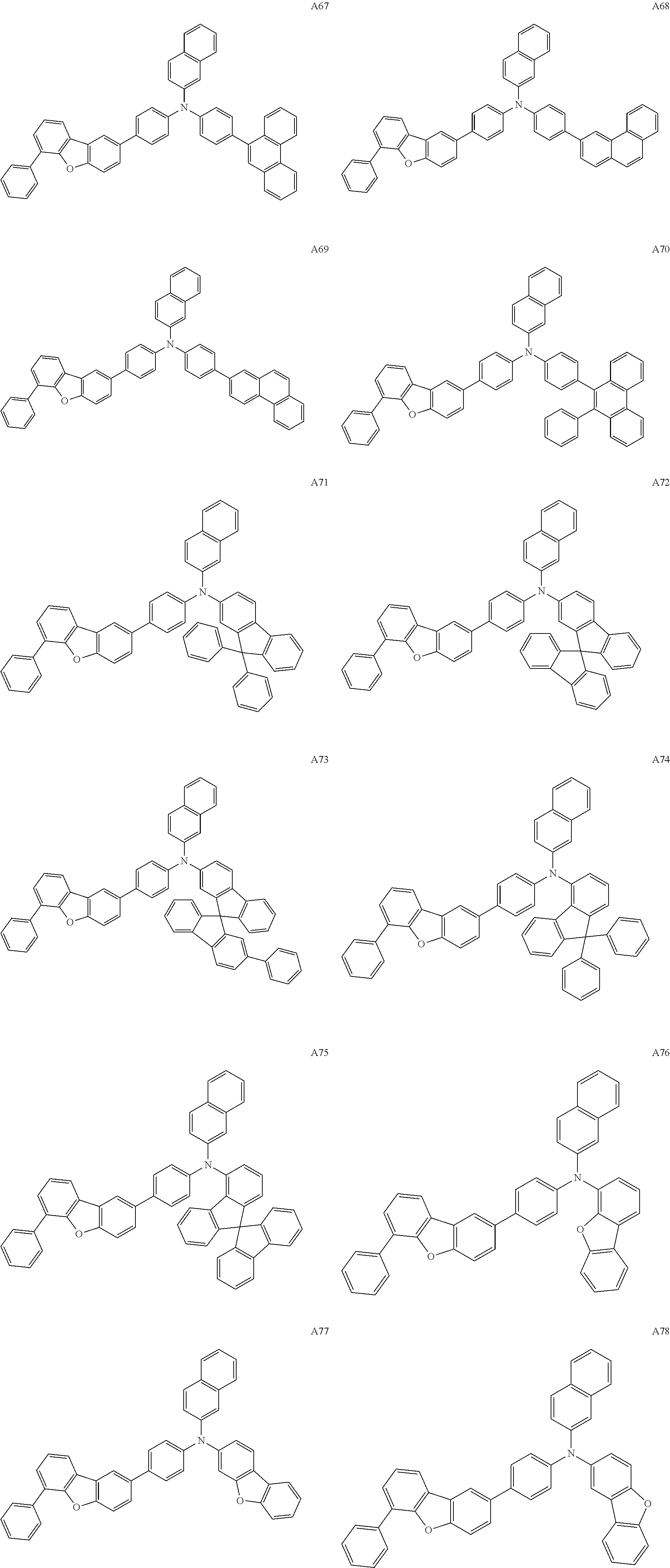

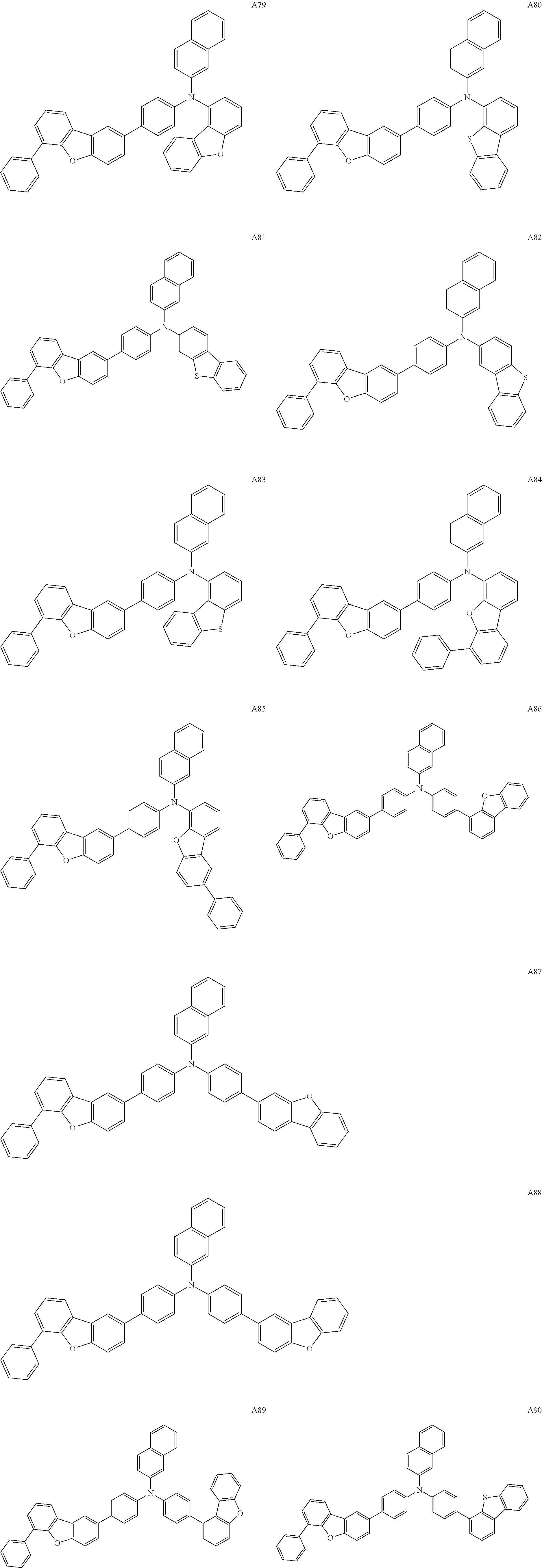

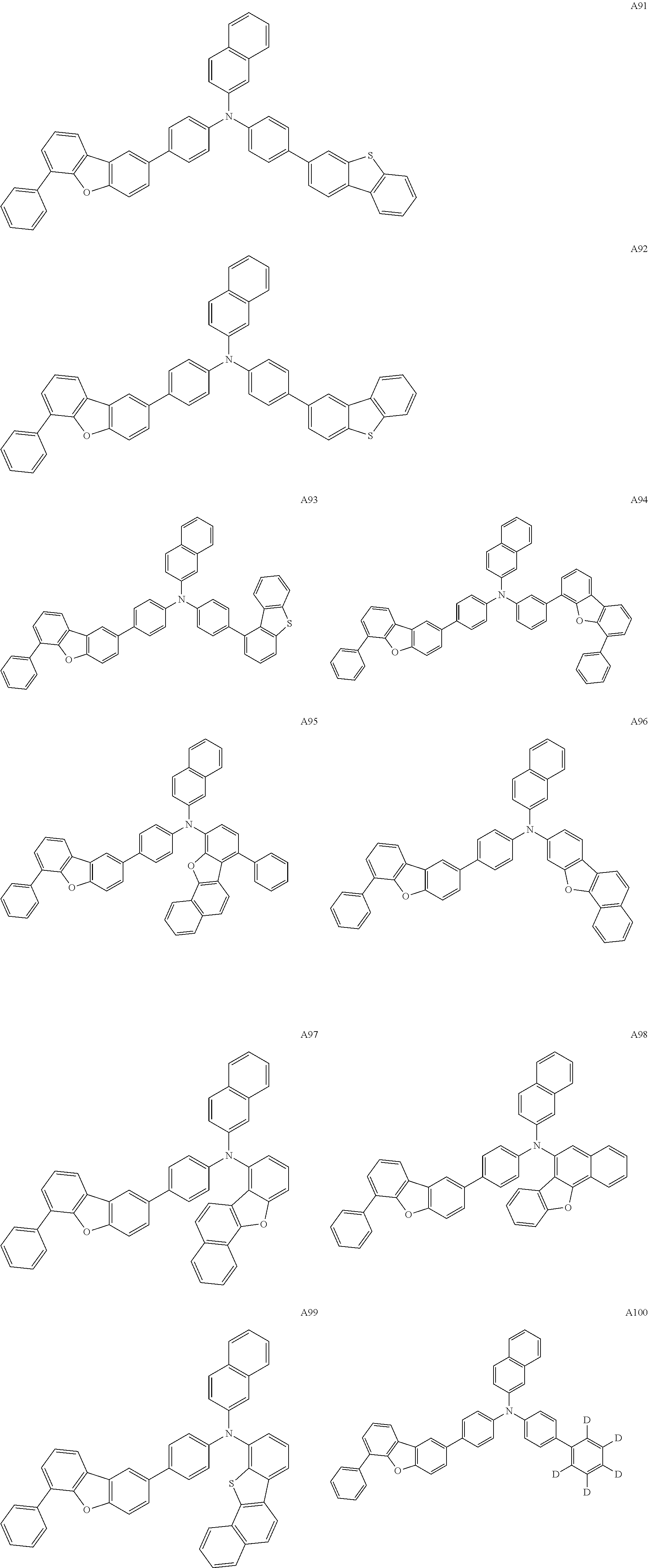

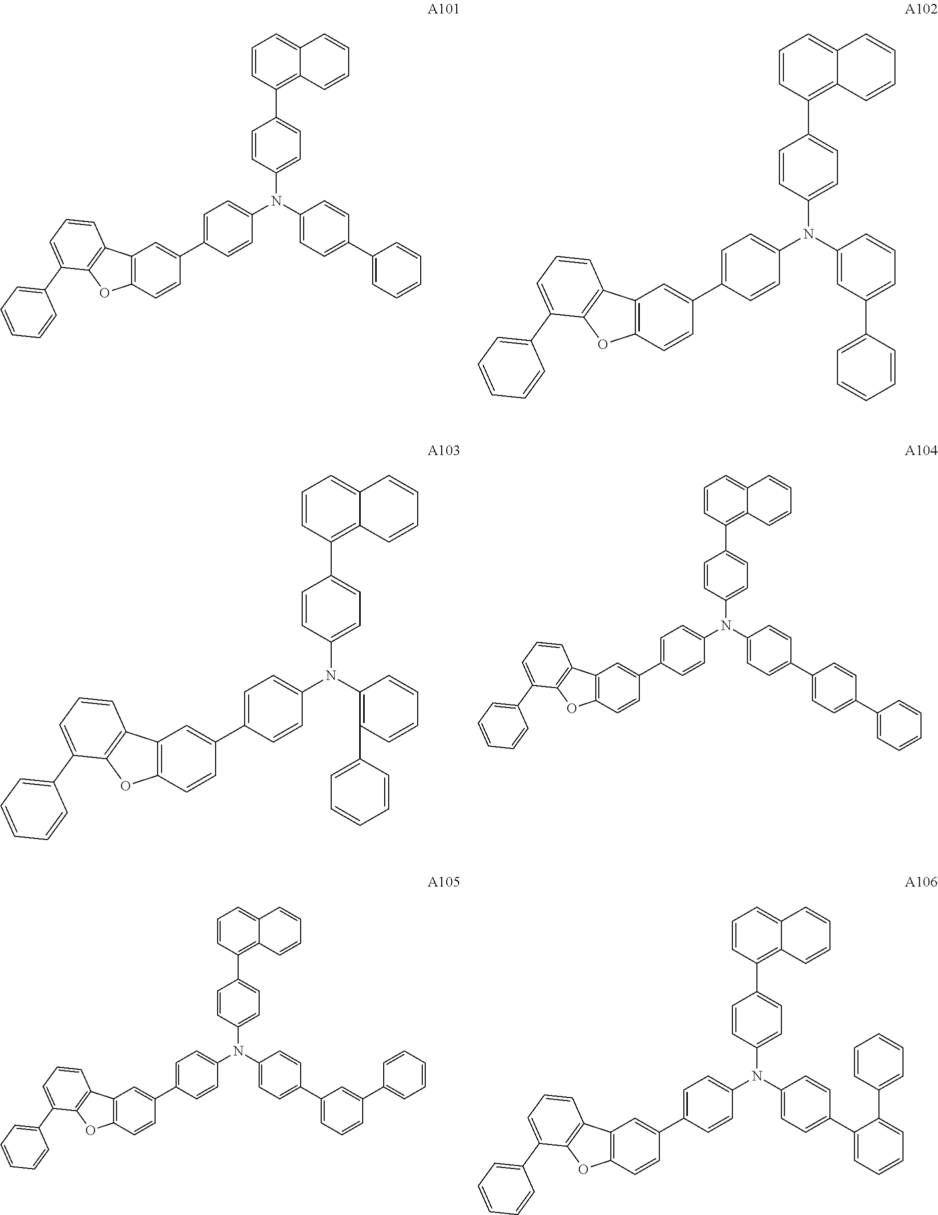

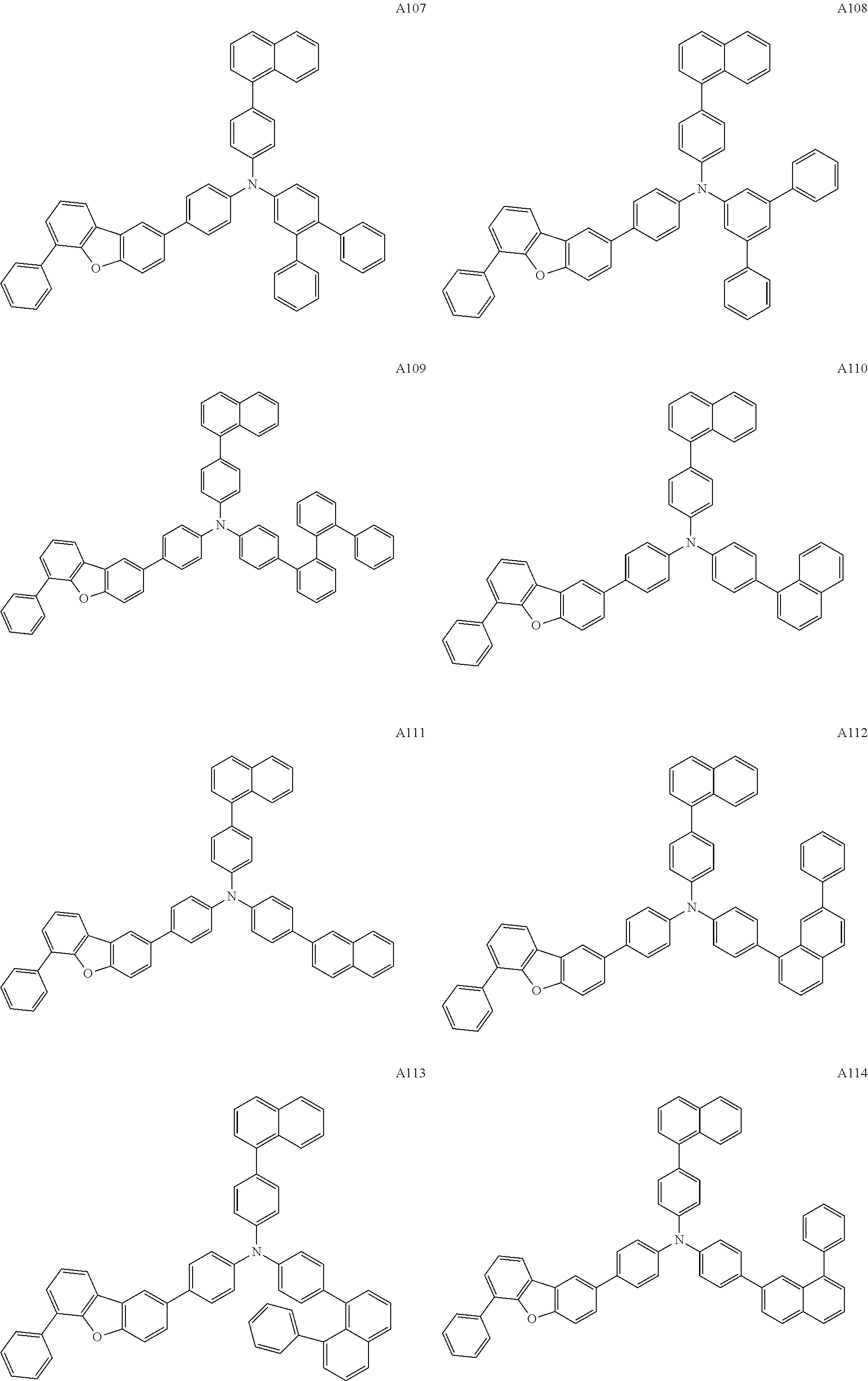

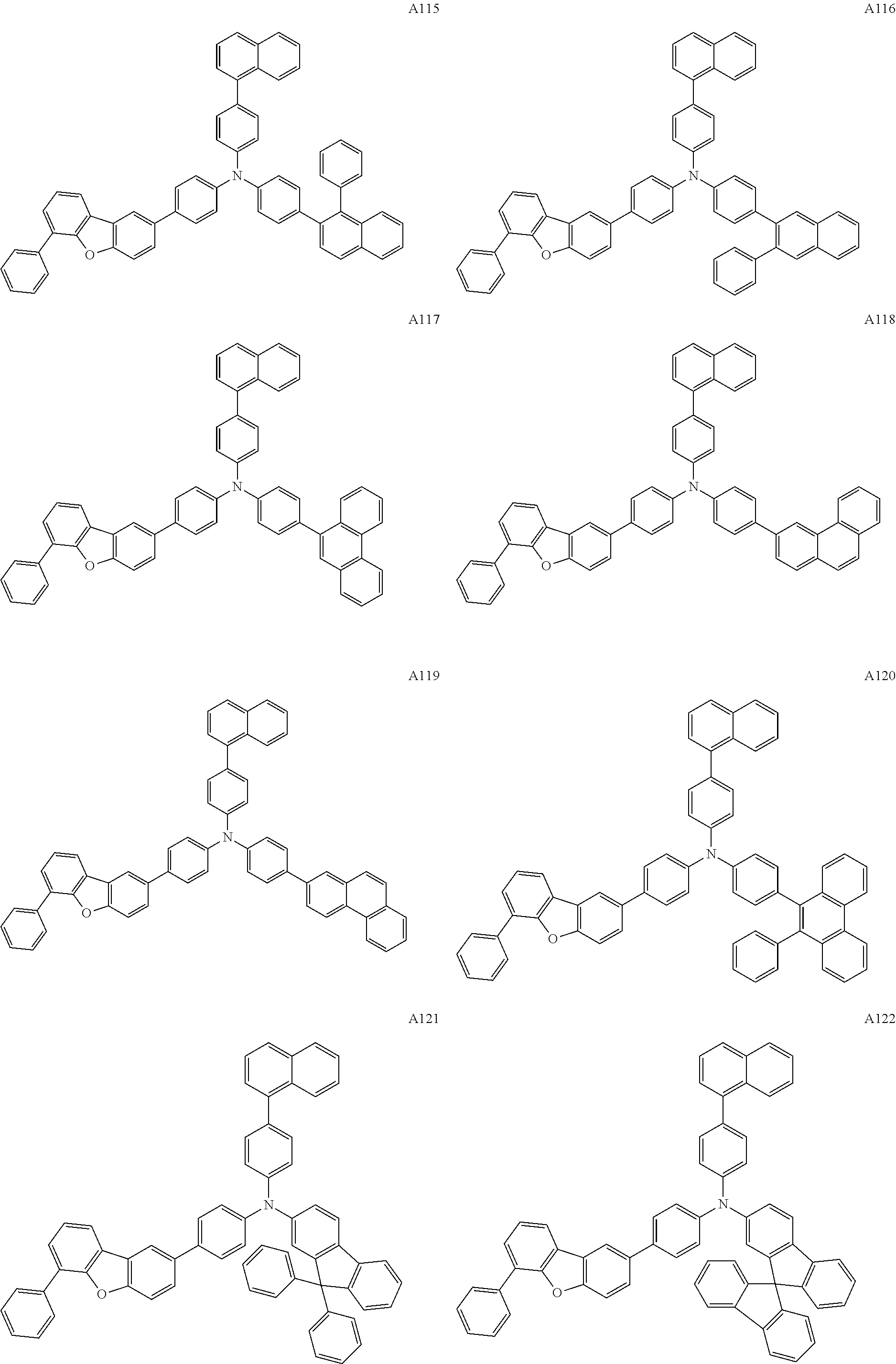

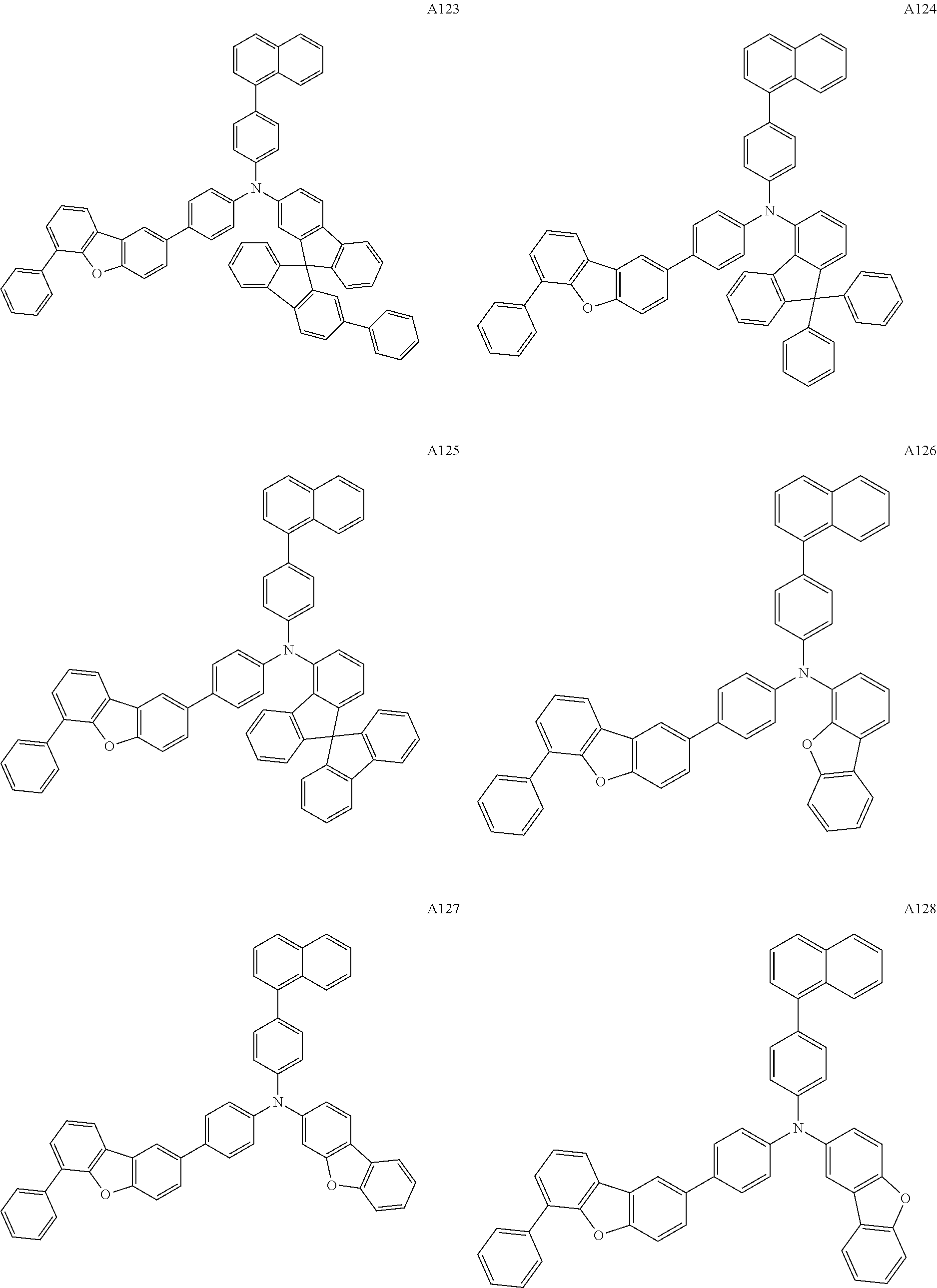

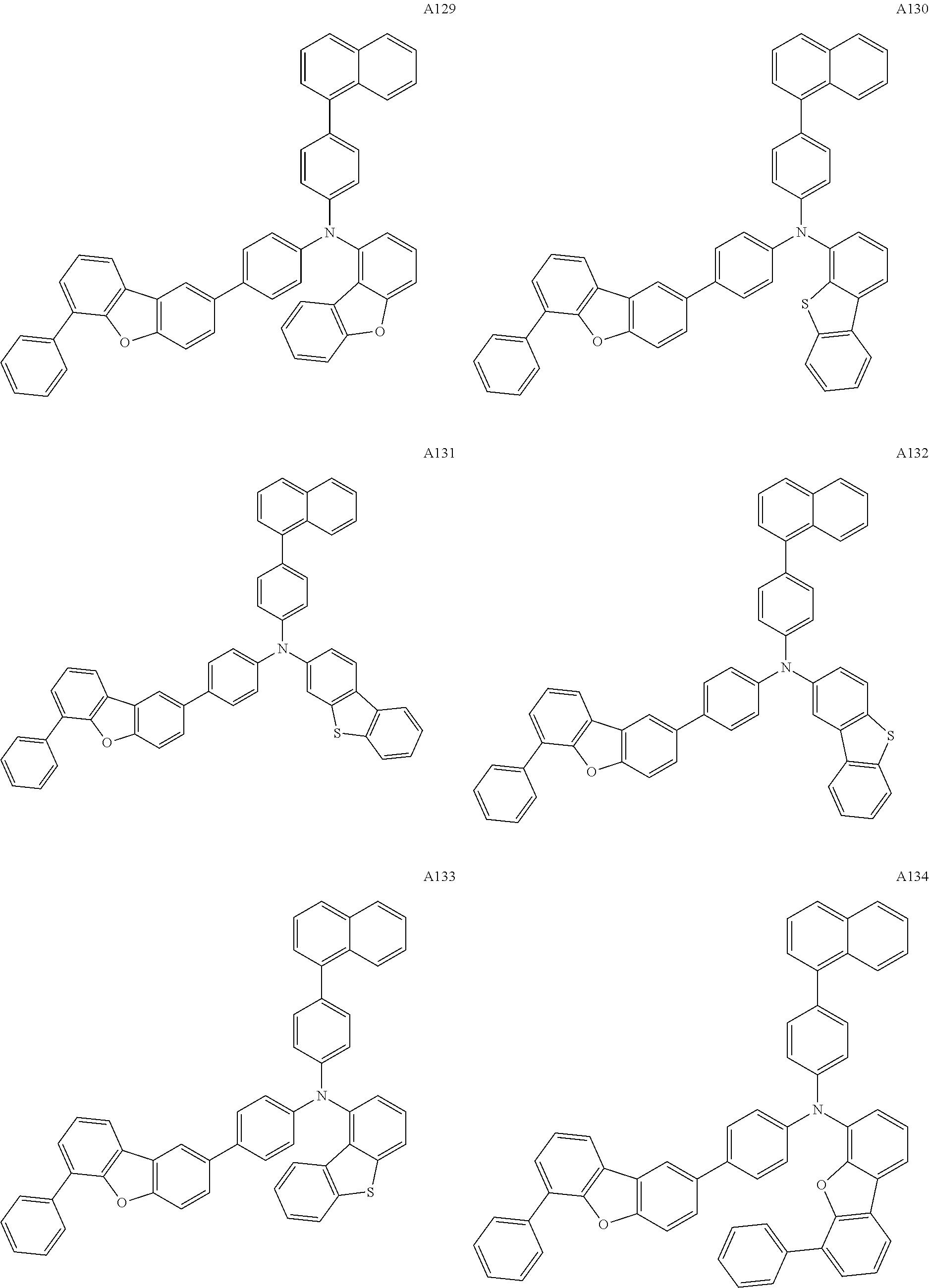

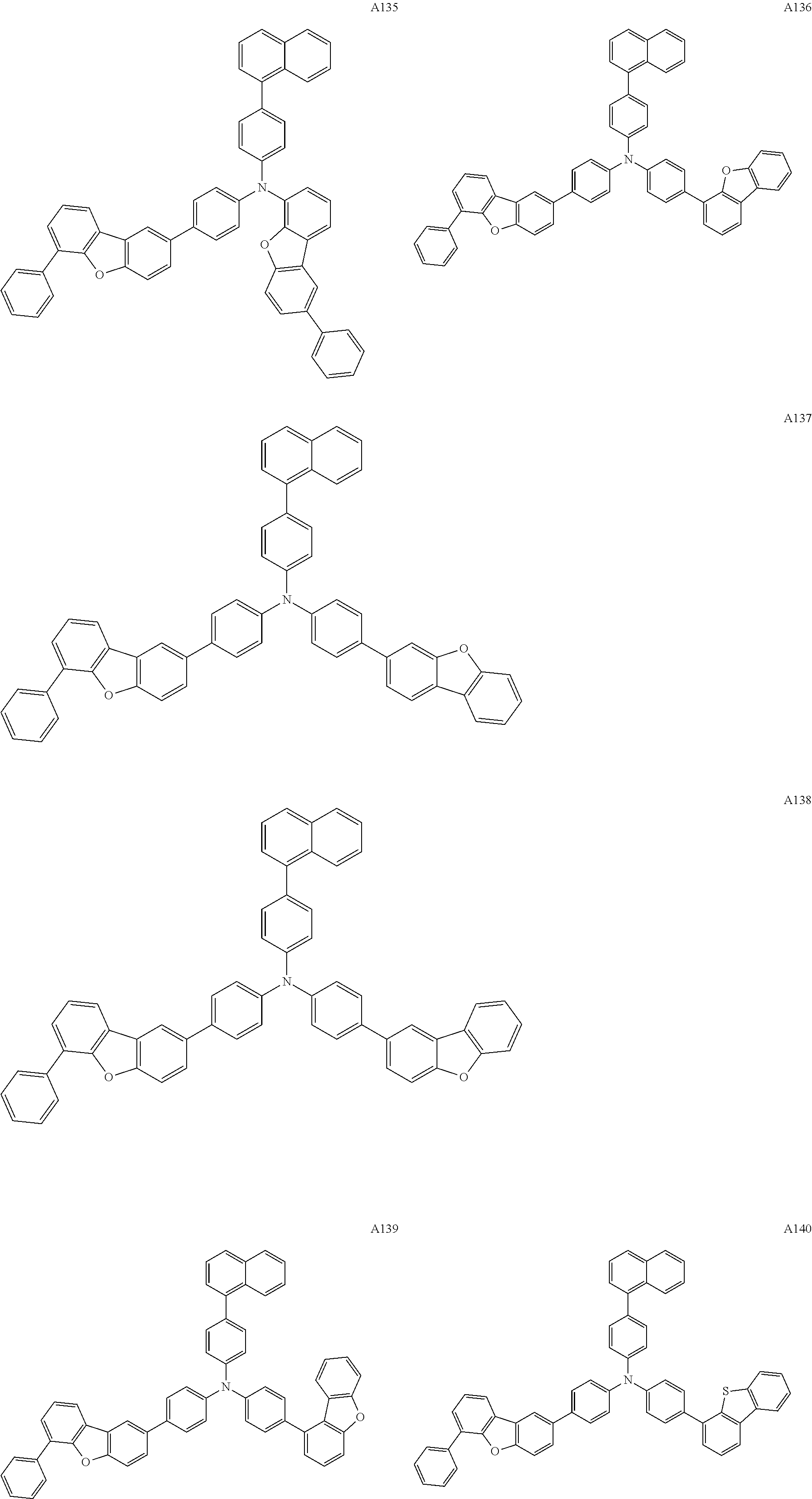

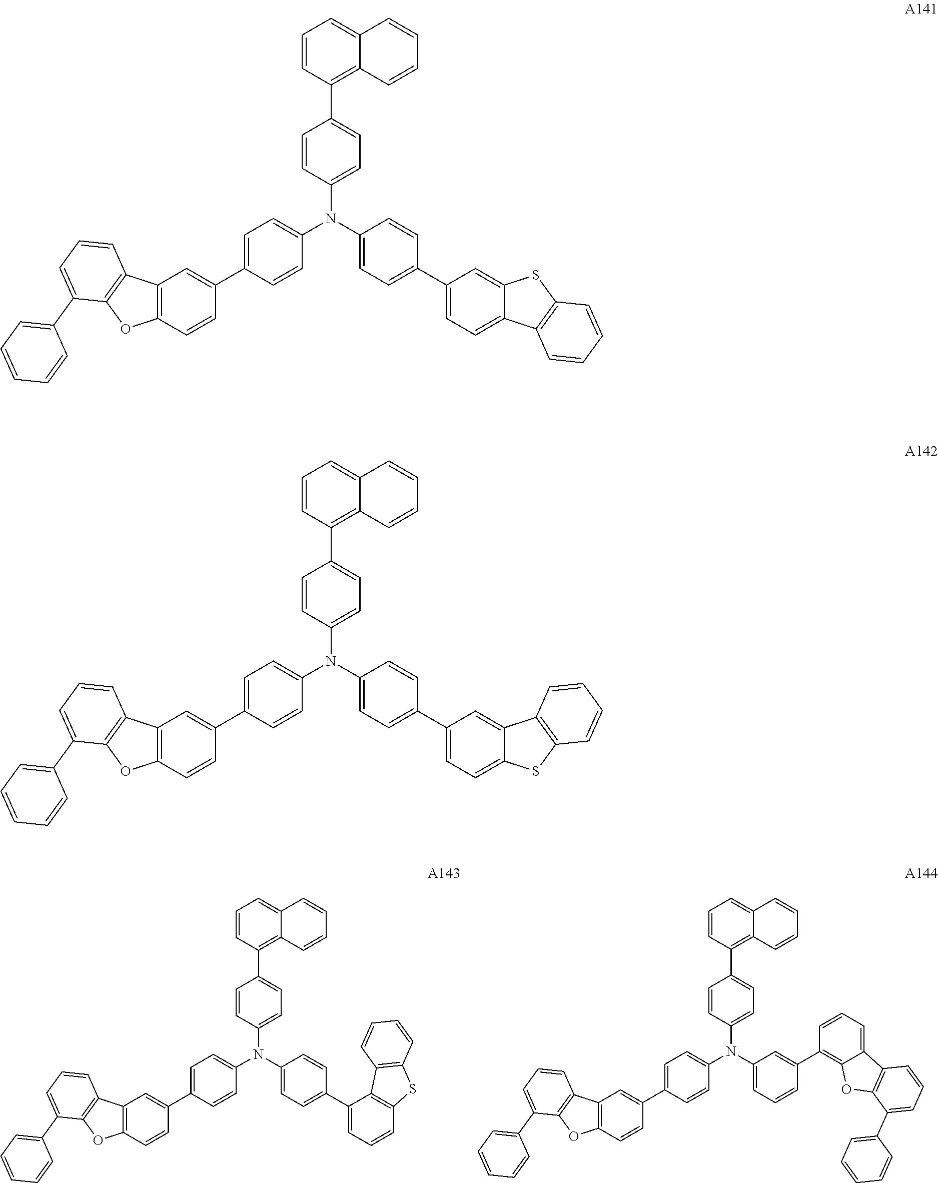 [SMILES: c1ccc(-c2cccc3c2oc2ccc(-c4ccc(N(c5ccc(-c6ccc7c(c6)sc6ccccc67)cc5)c5ccc(-c6cccc7ccccc67)cc5)cc4)cc23)cc1 ; c1ccc(-c2cccc3c2oc2ccc(-c4ccc(N(c5ccc(-c6ccc7sc8ccccc8c7c6)cc5)c5ccc(-c6cccc7ccccc67)cc5)cc4)cc23)cc1 ; c1ccc(-c2cccc3c2oc2ccc(-c4ccc(N(c5ccc(-c6cccc7ccccc67)cc5)c5ccc(-c6cccc7sc8ccccc8c67)cc5)cc4)cc23)cc1 ; c1ccc(-c2cccc3c2oc2ccc(-c4ccc(N(c5ccc(-c6cccc7ccccc67)cc5)c5cccc(-c6cccc7c6oc6c(-c8ccccc8)cccc67)c5)cc4)cc23)cc1]

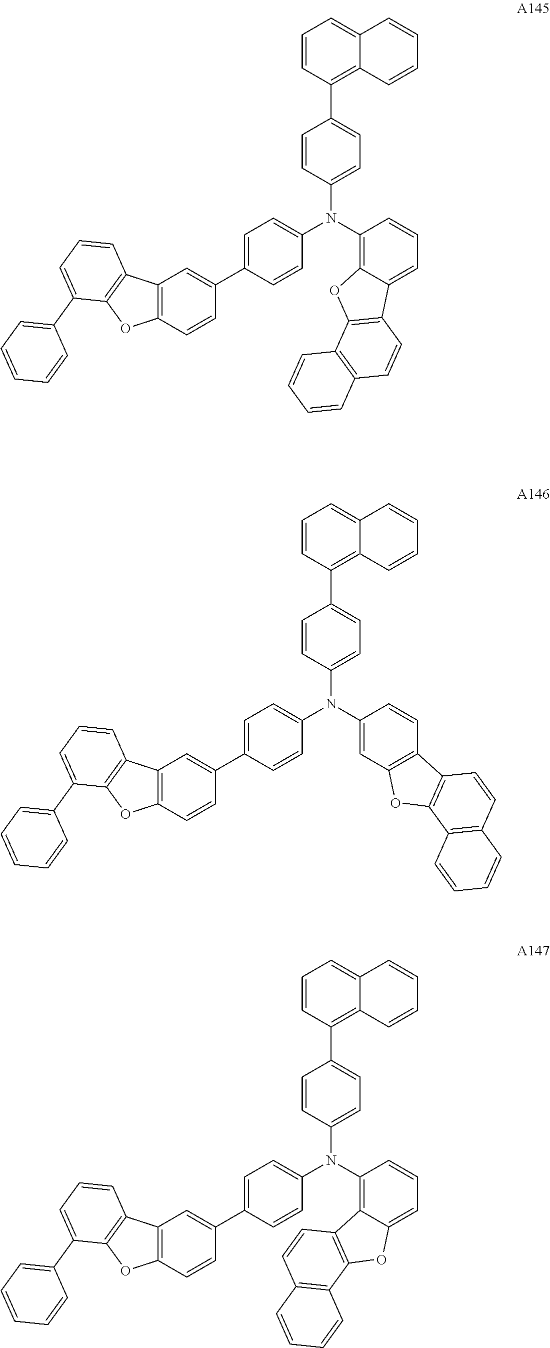 [SMILES: c1ccc(-c2cccc3c2oc2ccc(-c4ccc(N(c5ccc(-c6cccc7ccccc67)cc5)c5cccc6c5oc5c7ccccc7ccc65)cc4)cc23)cc1 ; c1ccc(-c2cccc3c2oc2ccc(-c4ccc(N(c5ccc(-c6cccc7ccccc67)cc5)c5ccc6c(c5)oc5c7ccccc7ccc65)cc4)cc23)cc1 ; c1ccc(-c2cccc3c2oc2ccc(-c4ccc(N(c5ccc(-c6cccc7ccccc67)cc5)c5cccc6oc7c8ccccc8ccc7c56)cc4)cc23)cc1]

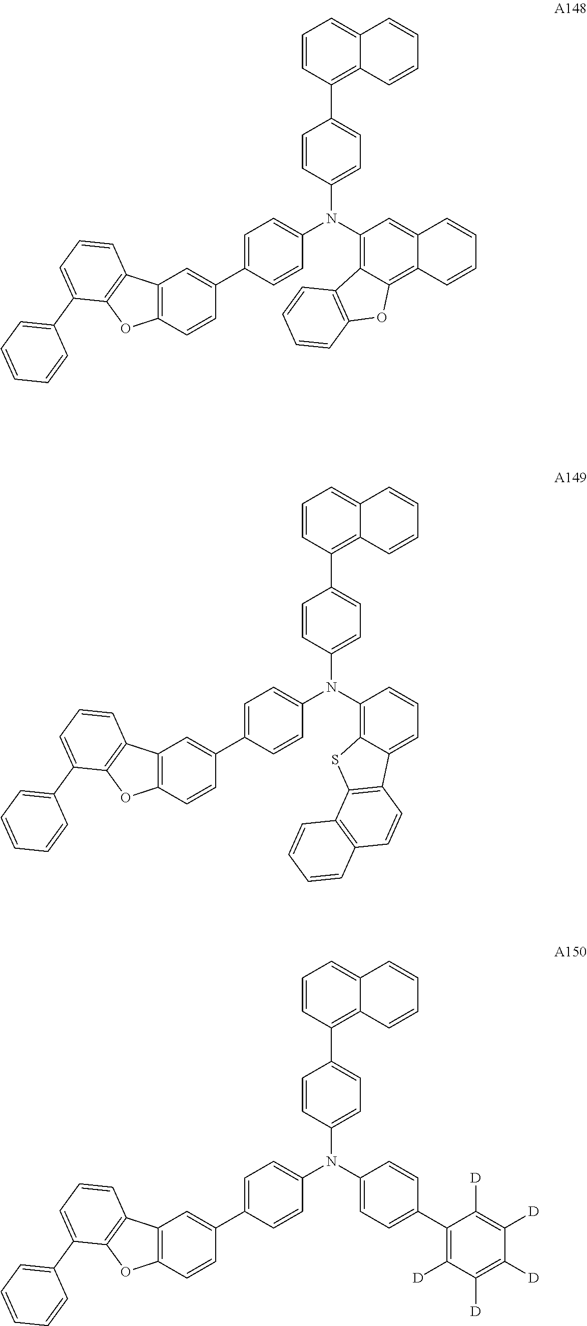 [SMILES: c1ccc(-c2cccc3c2oc2ccc(-c4ccc(N(c5ccc(-c6cccc7ccccc67)cc5)c5cc6ccccc6c6oc7ccccc7c56)cc4)cc23)cc1 ; c1ccc(-c2cccc3c2oc2ccc(-c4ccc(N(c5ccc(-c6cccc7ccccc67)cc5)c5cccc6c5sc5c7ccccc7ccc65)cc4)cc23)cc1 ; [2H]c1c([2H])c([2H])c(-c2ccc(N(c3ccc(-c4ccc5oc6c(-c7ccccc7)cccc6c5c4)cc3)c3ccc(-c4cccc5ccccc45)cc3)cc2)c([2H])c1[2H]]

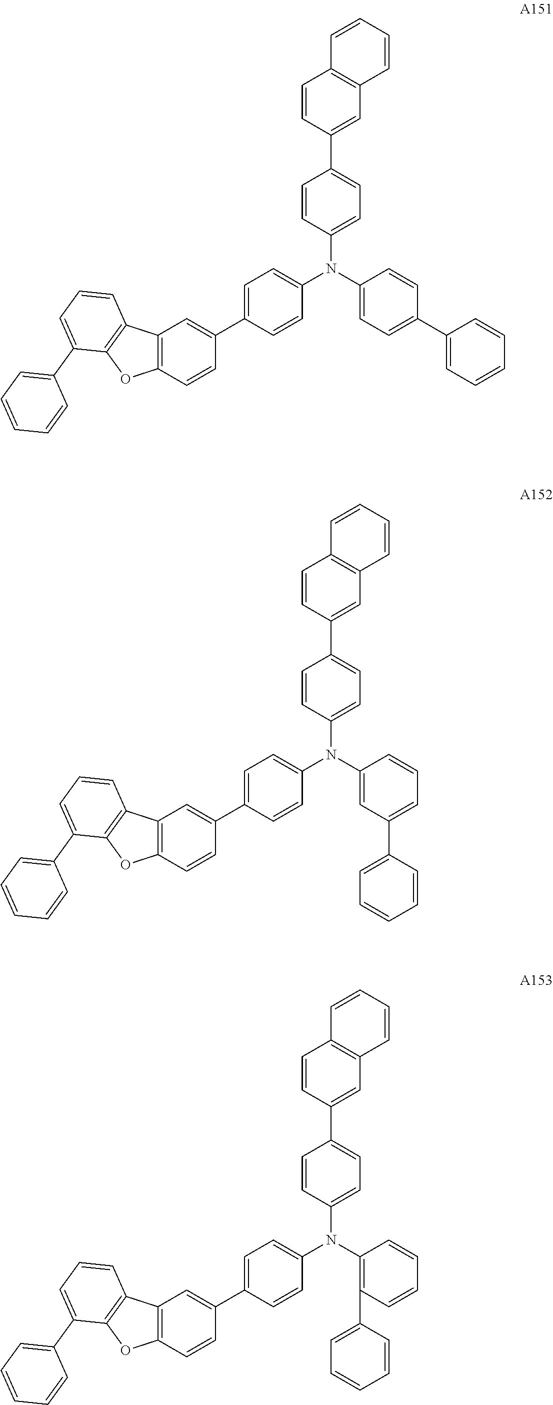 [SMILES: c1ccc(-c2ccc(N(c3ccc(-c4ccc5ccccc5c4)cc3)c3ccc(-c4ccc5oc6c(-c7ccccc7)cccc6c5c4)cc3)cc2)cc1 ; c1ccc(-c2cccc(N(c3ccc(-c4ccc5ccccc5c4)cc3)c3ccc(-c4ccc5oc6c(-c7ccccc7)cccc6c5c4)cc3)c2)cc1 ; c1ccc(-c2ccccc2N(c2ccc(-c3ccc4ccccc4c3)cc2)c2ccc(-c3ccc4oc5c(-c6ccccc6)cccc5c4c3)cc2)cc1]

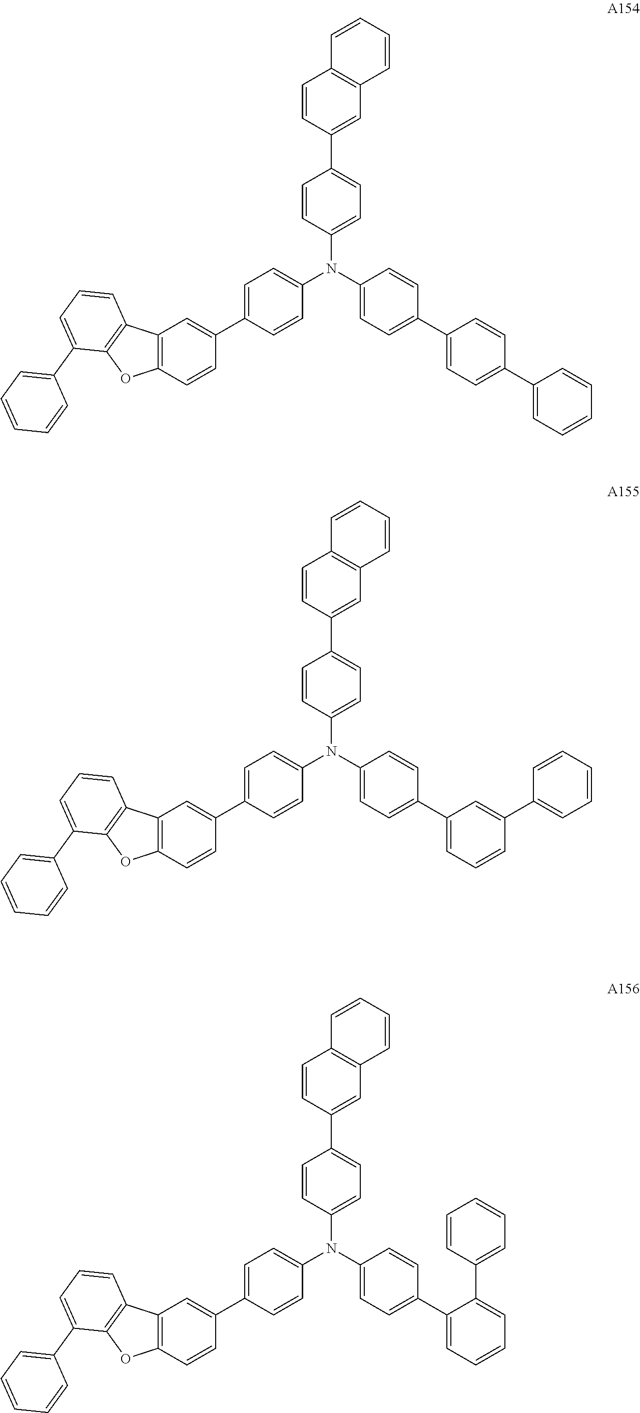 [SMILES: c1ccc(-c2ccc(-c3ccc(N(c4ccc(-c5ccc6ccccc6c5)cc4)c4ccc(-c5ccc6oc7c(-c8ccccc8)cccc7c6c5)cc4)cc3)cc2)cc1 ; c1ccc(-c2cccc(-c3ccc(N(c4ccc(-c5ccc6ccccc6c5)cc4)c4ccc(-c5ccc6oc7c(-c8ccccc8)cccc7c6c5)cc4)cc3)c2)cc1 ; c1ccc(-c2ccccc2-c2ccc(N(c3ccc(-c4ccc5ccccc5c4)cc3)c3ccc(-c4ccc5oc6c(-c7ccccc7)cccc6c5c4)cc3)cc2)cc1]

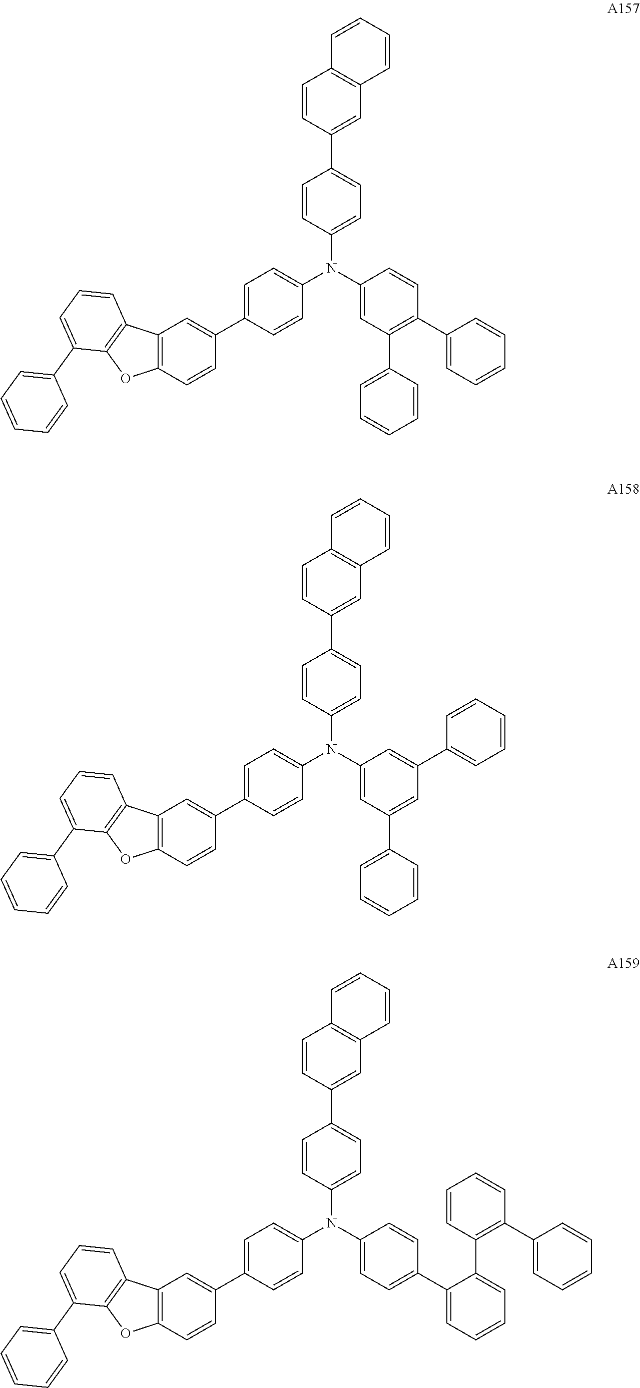 [SMILES: c1ccc(-c2ccc(N(c3ccc(-c4ccc5ccccc5c4)cc3)c3ccc(-c4ccc5oc6c(-c7ccccc7)cccc6c5c4)cc3)cc2-c2ccccc2)cc1 ; c1ccc(-c2cc(-c3ccccc3)cc(N(c3ccc(-c4ccc5ccccc5c4)cc3)c3ccc(-c4ccc5oc6c(-c7ccccc7)cccc6c5c4)cc3)c2)cc1 ; c1ccc(-c2ccccc2-c2ccccc2-c2ccc(N(c3ccc(-c4ccc5ccccc5c4)cc3)c3ccc(-c4ccc5oc6c(-c7ccccc7)cccc6c5c4)cc3)cc2)cc1]

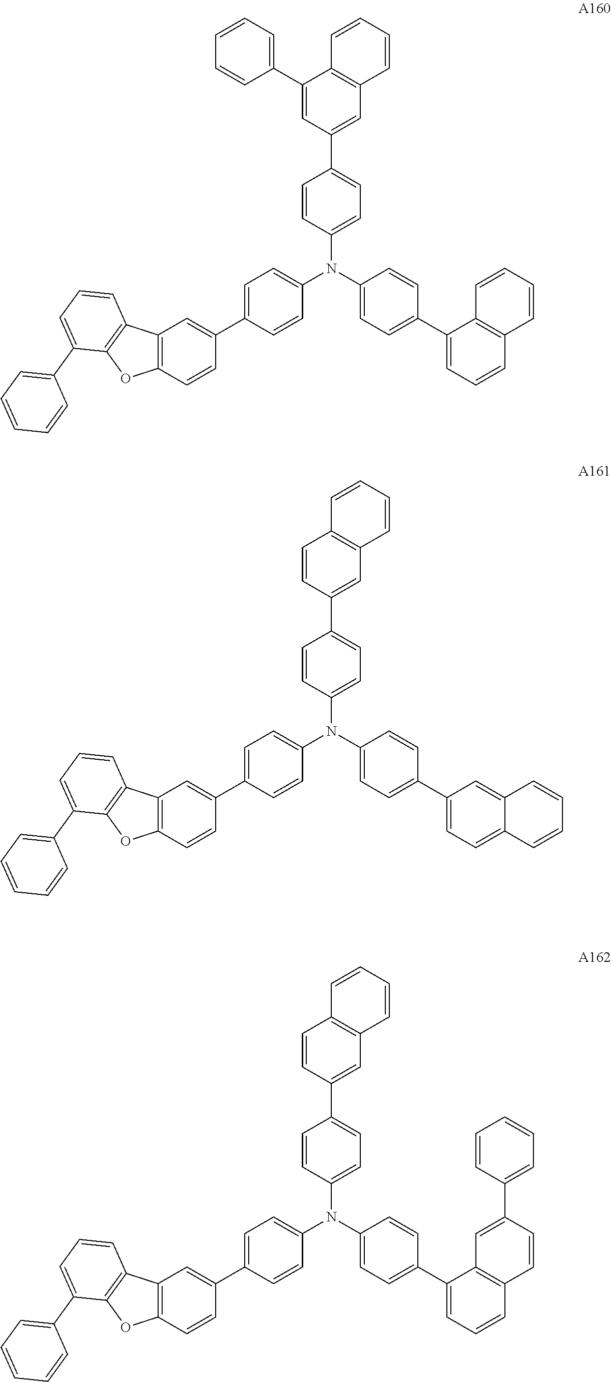 [SMILES: c1ccc(-c2cc(-c3ccc(N(c4ccc(-c5ccc6oc7c(-c8ccccc8)cccc7c6c5)cc4)c4ccc(-c5cccc6ccccc56)cc4)cc3)cc3ccccc23)cc1 ; c1ccc(-c2cccc3c2oc2ccc(-c4ccc(N(c5ccc(-c6ccc7ccccc7c6)cc5)c5ccc(-c6ccc7ccccc7c6)cc5)cc4)cc23)cc1 ; c1ccc(-c2ccc3cccc(-c4ccc(N(c5ccc(-c6ccc7ccccc7c6)cc5)c5ccc(-c6ccc7oc8c(-c9ccccc9)cccc8c7c6)cc5)cc4)c3c2)cc1]

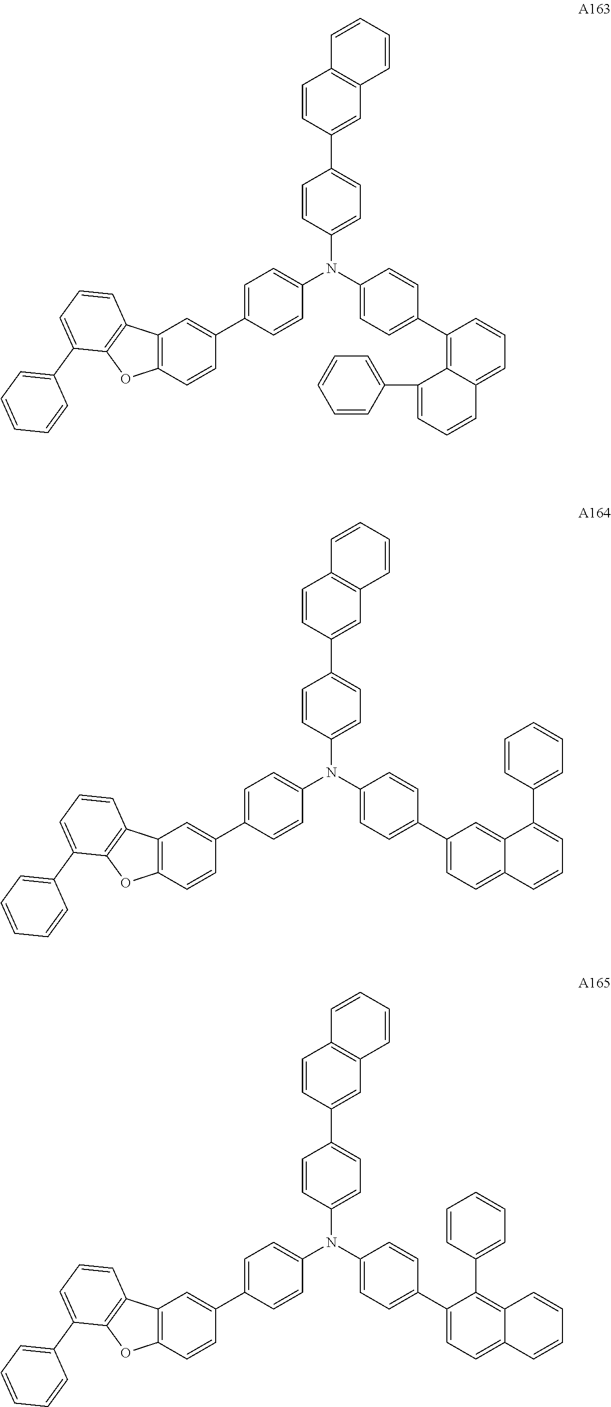 [SMILES: c1ccc(-c2cccc3c2oc2ccc(-c4ccc(N(c5ccc(-c6ccc7ccccc7c6)cc5)c5ccc(-c6cccc7cccc(-c8ccccc8)c67)cc5)cc4)cc23)cc1 ; c1ccc(-c2cccc3ccc(-c4ccc(N(c5ccc(-c6ccc7ccccc7c6)cc5)c5ccc(-c6ccc7oc8c(-c9ccccc9)cccc8c7c6)cc5)cc4)cc23)cc1 ; c1ccc(-c2c(-c3ccc(N(c4ccc(-c5ccc6ccccc6c5)cc4)c4ccc(-c5ccc6oc7c(-c8ccccc8)cccc7c6c5)cc4)cc3)ccc3ccccc23)cc1]

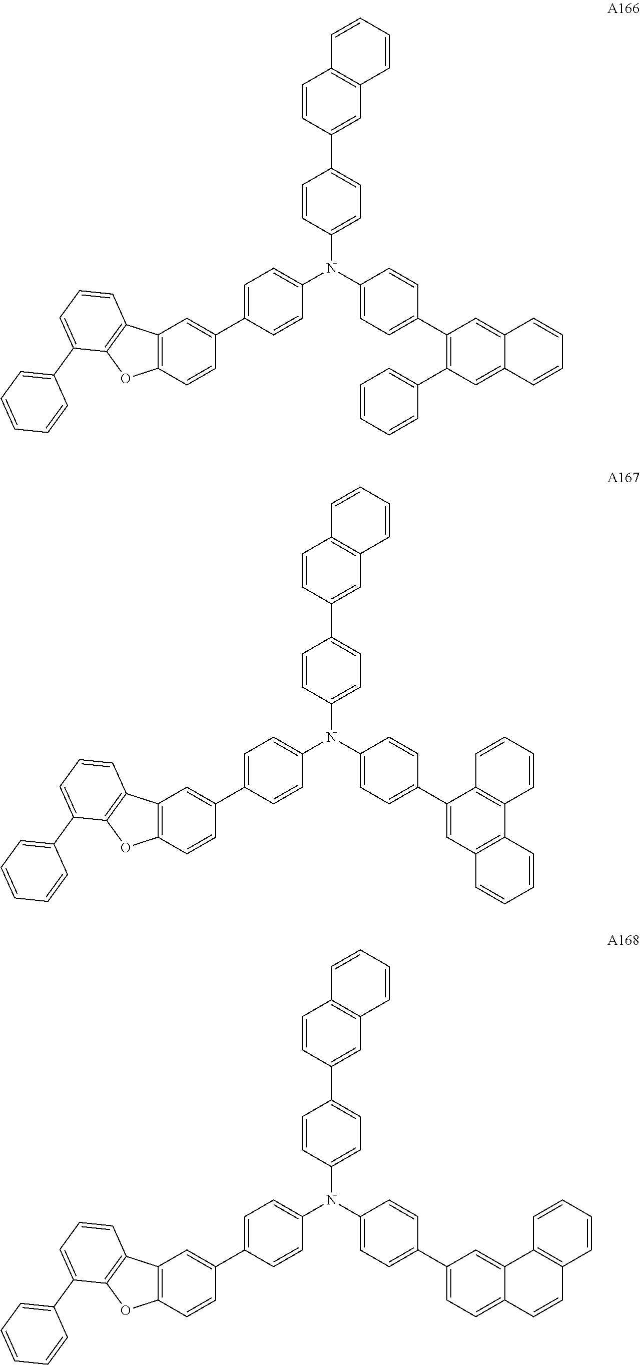 [SMILES: c1ccc(-c2cc3ccccc3cc2-c2ccc(N(c3ccc(-c4ccc5ccccc5c4)cc3)c3ccc(-c4ccc5oc6c(-c7ccccc7)cccc6c5c4)cc3)cc2)cc1 ; c1ccc(-c2cccc3c2oc2ccc(-c4ccc(N(c5ccc(-c6ccc7ccccc7c6)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7ccccc7c6)cc5)c5ccc(-c6ccc7ccc8ccccc8c7c6)cc5)cc4)cc23)cc1]

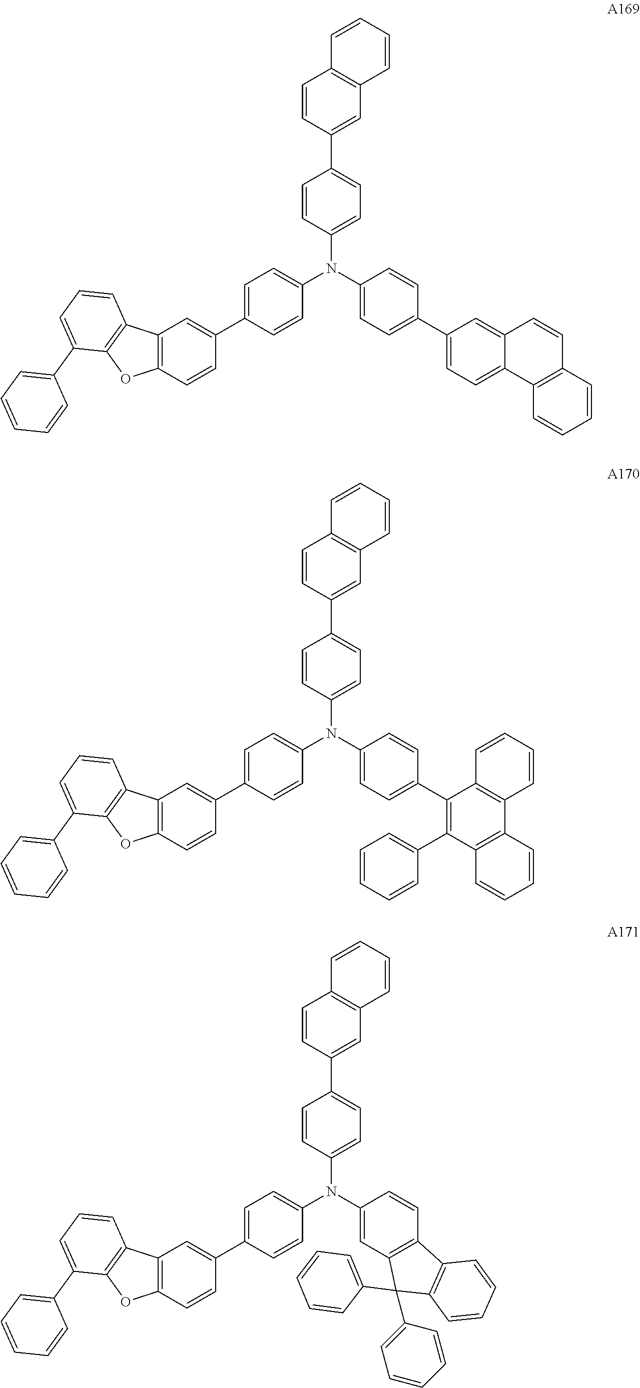 [SMILES: c1ccc(-c2cccc3c2oc2ccc(-c4ccc(N(c5ccc(-c6ccc7ccccc7c6)cc5)c5ccc(-c6ccc7c(ccc8ccccc87)c6)cc5)cc4)cc23)cc1 ; c1ccc(-c2c(-c3ccc(N(c4ccc(-c5ccc6ccccc6c5)cc4)c4ccc(-c5ccc6oc7c(-c8ccccc8)cccc7c6c5)cc4)cc3)c3ccccc3c3ccccc23)cc1 ; c1ccc(-c2cccc3c2oc2ccc(-c4ccc(N(c5ccc(-c6ccc7ccccc7c6)cc5)c5ccc6c(c5)C(c5ccccc5)(c5ccccc5)c5ccccc5-6)cc4)cc23)cc1]

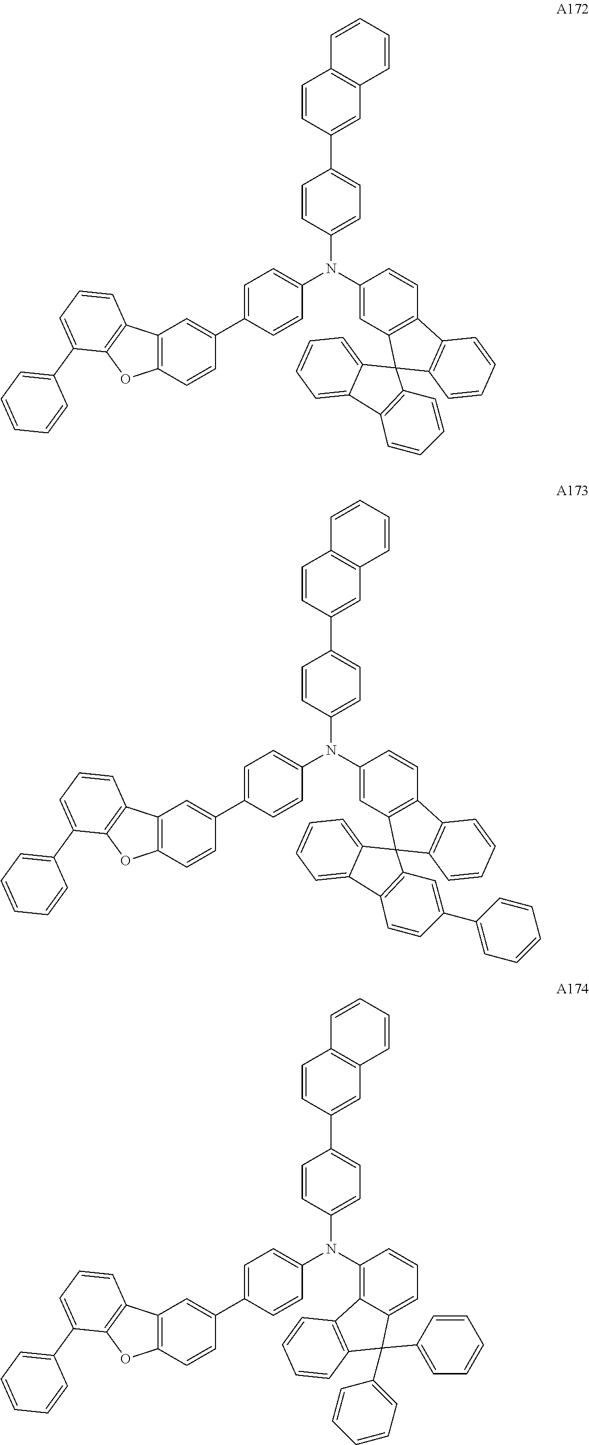 [SMILES: c1ccc(-c2cccc3c2oc2ccc(-c4ccc(N(c5ccc(-c6ccc7ccccc7c6)cc5)c5ccc6c(c5)C5(c7ccccc7-c7ccccc75)c5ccccc5-6)cc4)cc23)cc1 ; c1ccc(-c2ccc3c(c2)C2(c4ccccc4-3)c3ccccc3-c3ccc(N(c4ccc(-c5ccc6ccccc6c5)cc4)c4ccc(-c5ccc6oc7c(-c8ccccc8)cccc7c6c5)cc4)cc32)cc1 ; c1ccc(-c2cccc3c2oc2ccc(-c4ccc(N(c5ccc(-c6ccc7ccccc7c6)cc5)c5cccc6c5-c5ccccc5C6(c5ccccc5)c5ccccc5)cc4)cc23)cc1]

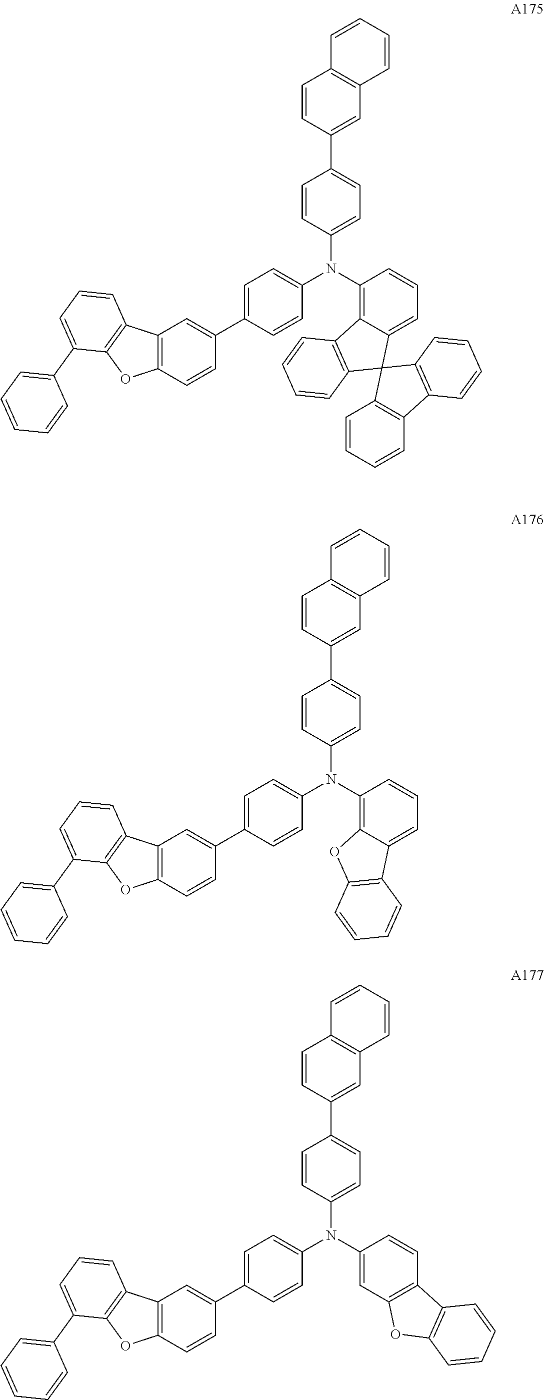

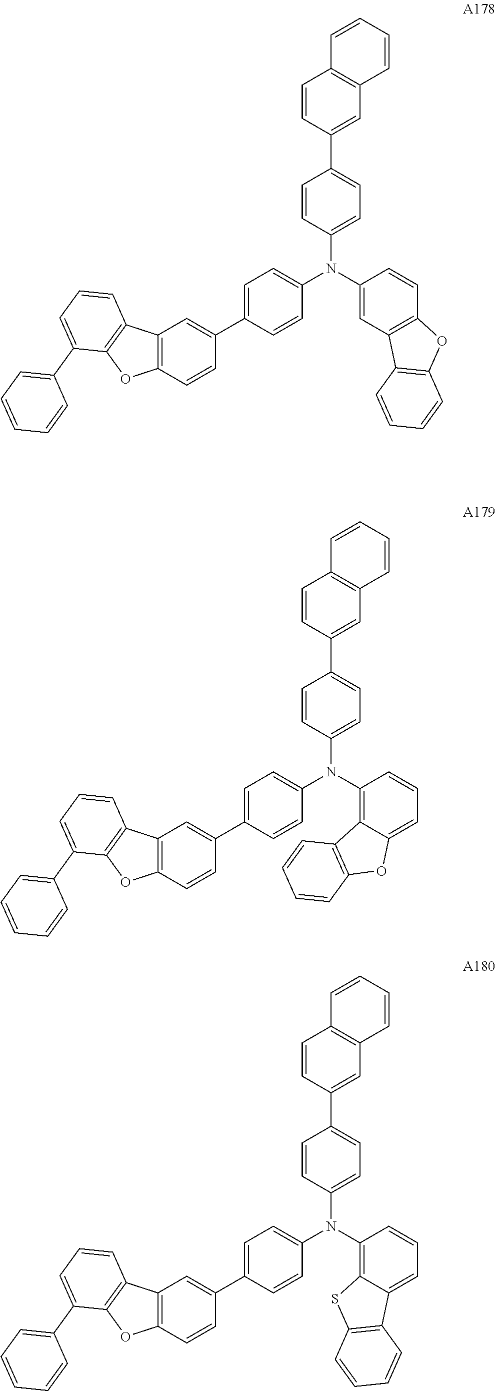 [SMILES: c1ccc(-c2cccc3c2oc2ccc(-c4ccc(N(c5ccc(-c6ccc7ccccc7c6)cc5)c5ccc6oc7ccccc7c6c5)cc4)cc23)cc1 ; c1ccc(-c2cccc3c2oc2ccc(-c4ccc(N(c5ccc(-c6ccc7ccccc7c6)cc5)c5cccc6oc7ccccc7c56)cc4)cc23)cc1 ; c1ccc(-c2cccc3c2oc2ccc(-c4ccc(N(c5ccc(-c6ccc7ccccc7c6)cc5)c5cccc6c5sc5ccccc56)cc4)cc23)cc1]

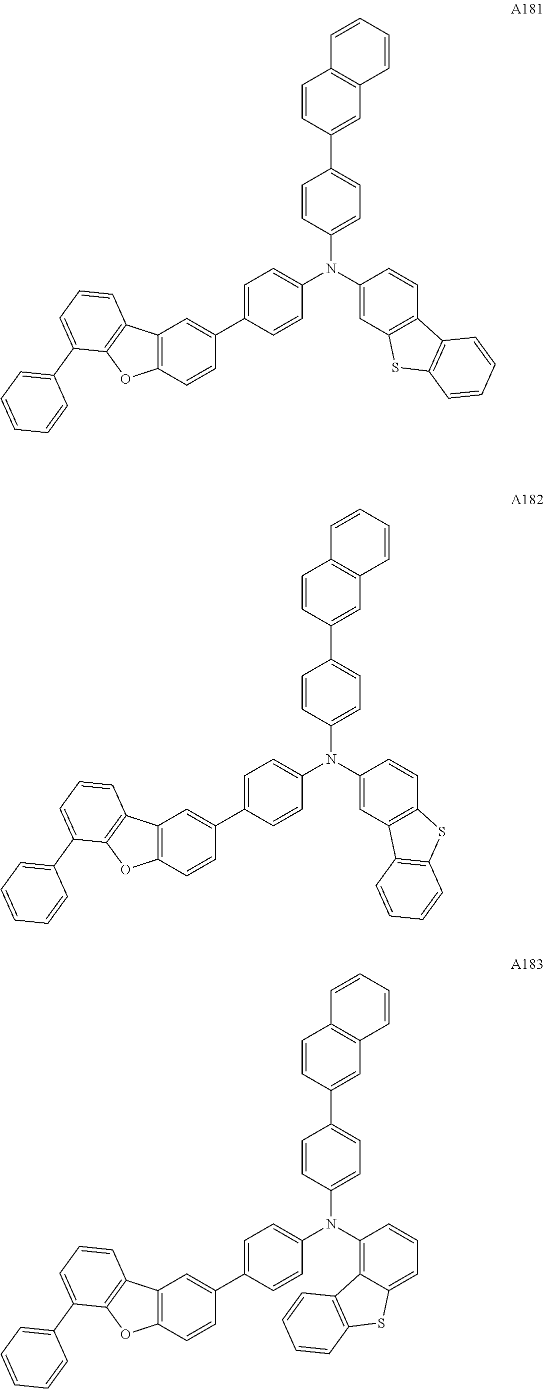 [SMILES: c1ccc(-c2cccc3c2oc2ccc(-c4ccc(N(c5ccc(-c6ccc7ccccc7c6)cc5)c5ccc6c(c5)sc5ccccc56)cc4)cc23)cc1 ; c1ccc(-c2cccc3c2oc2ccc(-c4ccc(N(c5ccc(-c6ccc7ccccc7c6)cc5)c5ccc6sc7ccccc7c6c5)cc4)cc23)cc1 ; c1ccc(-c2cccc3c2oc2ccc(-c4ccc(N(c5ccc(-c6ccc7ccccc7c6)cc5)c5cccc6sc7ccccc7c56)cc4)cc23)cc1]

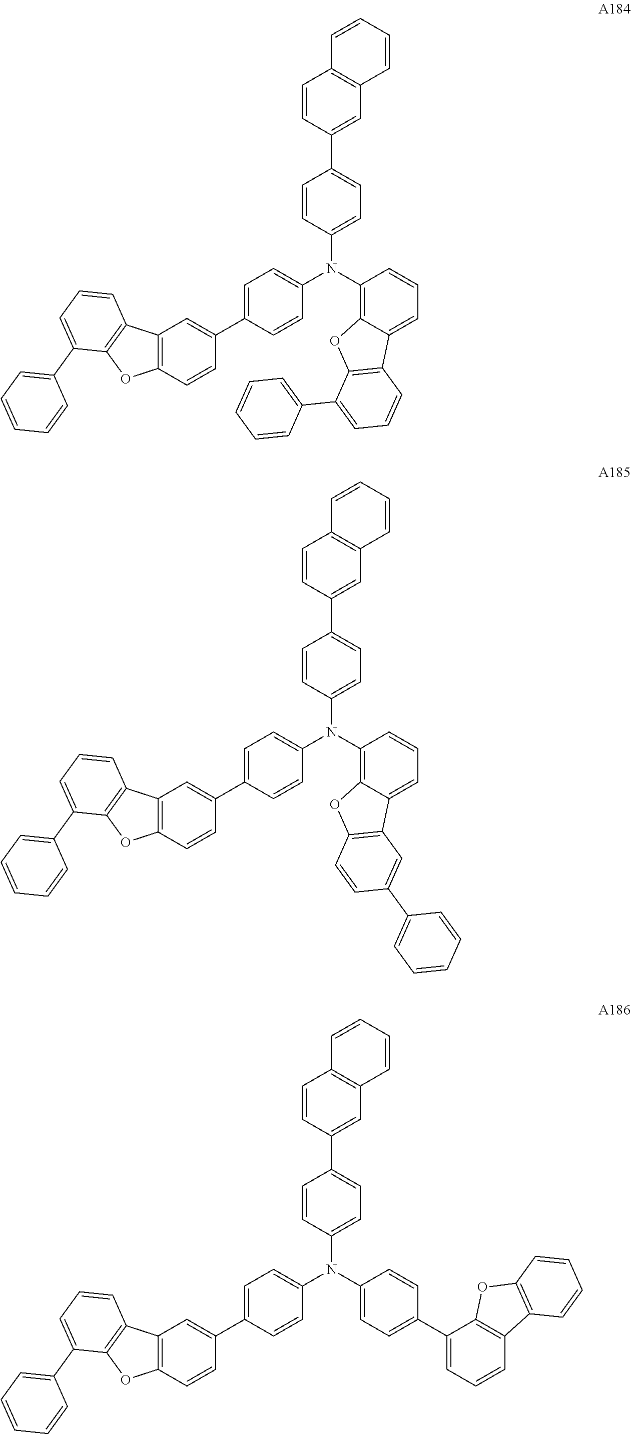 [SMILES: c1ccc(-c2cccc3c2oc2ccc(-c4ccc(N(c5ccc(-c6ccc7ccccc7c6)cc5)c5cccc6c5oc5c(-c7ccccc7)cccc56)cc4)cc23)cc1 ; c1ccc(-c2ccc3oc4c(N(c5ccc(-c6ccc7ccccc7c6)cc5)c5ccc(-c6ccc7oc8c(-c9ccccc9)cccc8c7c6)cc5)cccc4c3c2)cc1 ; c1ccc(-c2cccc3c2oc2ccc(-c4ccc(N(c5ccc(-c6ccc7ccccc7c6)cc5)c5ccc(-c6cccc7c6oc6ccccc67)cc5)cc4)cc23)cc1]

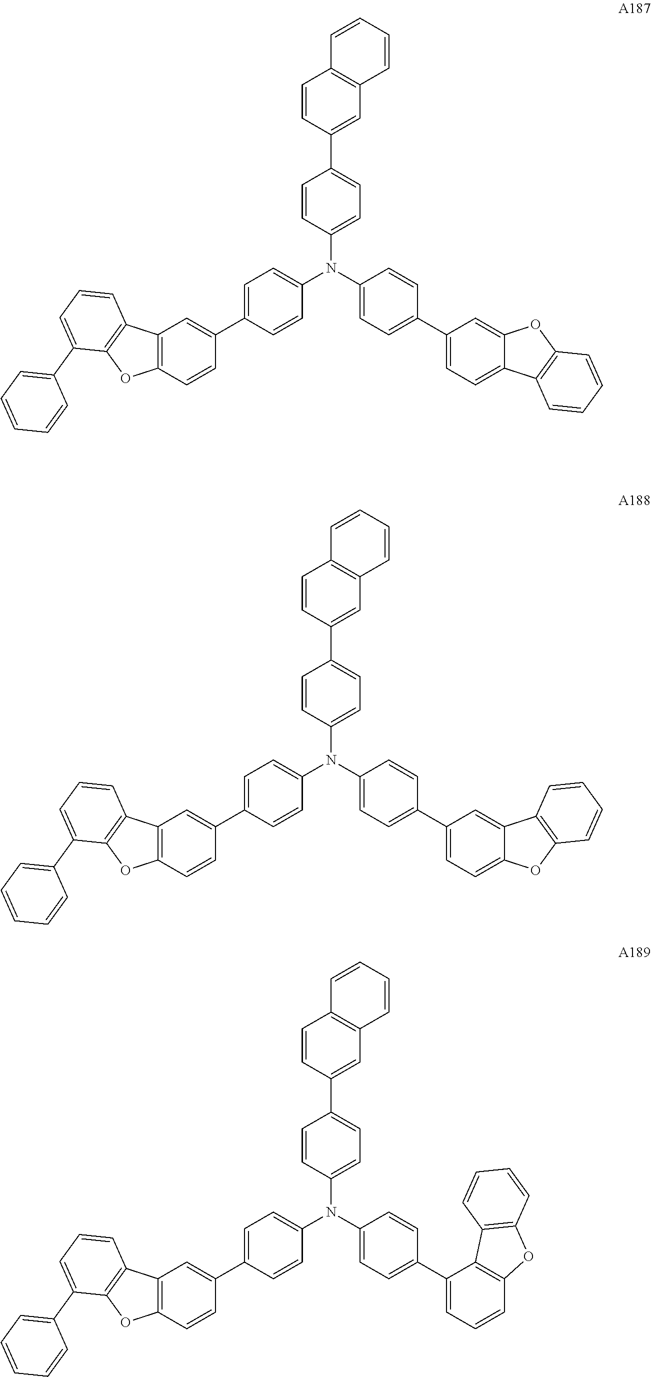 [SMILES: c1ccc(-c2cccc3c2oc2ccc(-c4ccc(N(c5ccc(-c6ccc7ccccc7c6)cc5)c5ccc(-c6ccc7c(c6)oc6ccccc67)cc5)cc4)cc23)cc1 ; c1ccc(-c2cccc3c2oc2ccc(-c4ccc(N(c5ccc(-c6ccc7ccccc7c6)cc5)c5ccc(-c6ccc7oc8ccccc8c7c6)cc5)cc4)cc23)cc1 ; c1ccc(-c2cccc3c2oc2ccc(-c4ccc(N(c5ccc(-c6ccc7ccccc7c6)cc5)c5ccc(-c6cccc7oc8ccccc8c67)cc5)cc4)cc23)cc1]

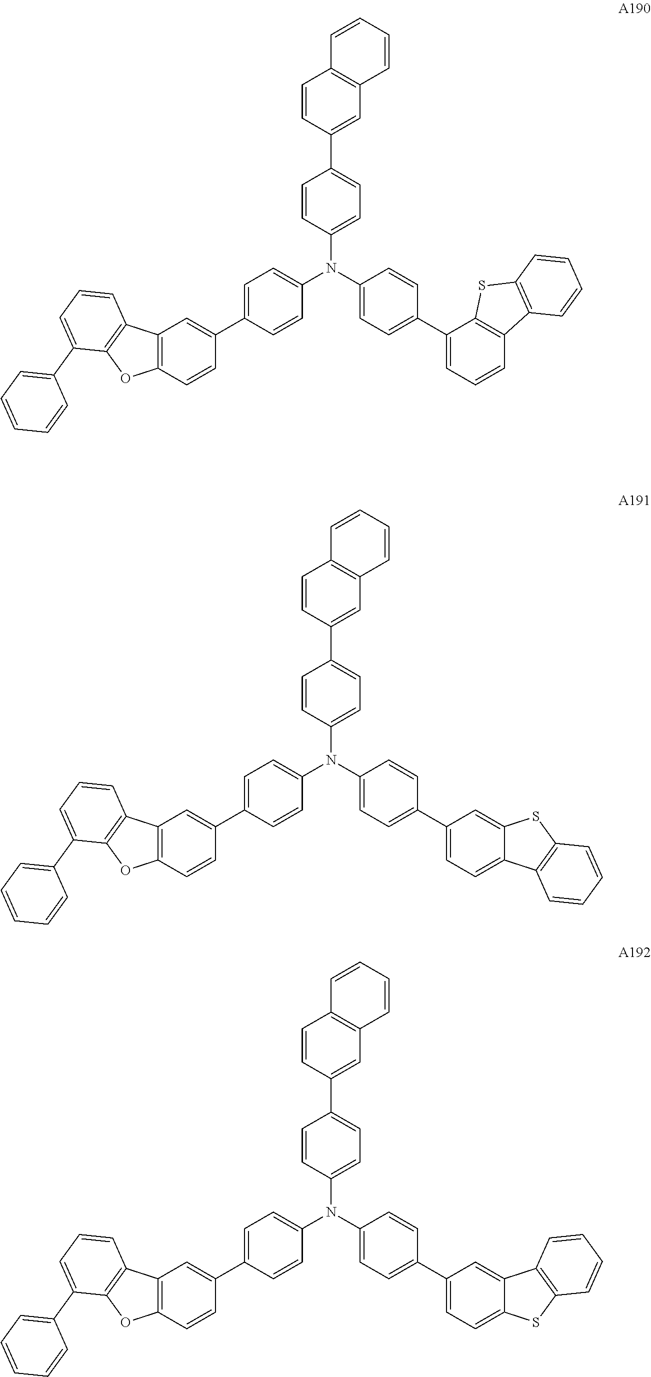 [SMILES: c1ccc(-c2cccc3c2oc2ccc(-c4ccc(N(c5ccc(-c6ccc7ccccc7c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7ccccc7c6)cc5)c5ccc(-c6ccc7c(c6)sc6ccccc67)cc5)cc4)cc23)cc1 ; c1ccc(-c2cccc3c2oc2ccc(-c4ccc(N(c5ccc(-c6ccc7ccccc7c6)cc5)c5ccc(-c6ccc7sc8ccccc8c7c6)cc5)cc4)cc23)cc1]

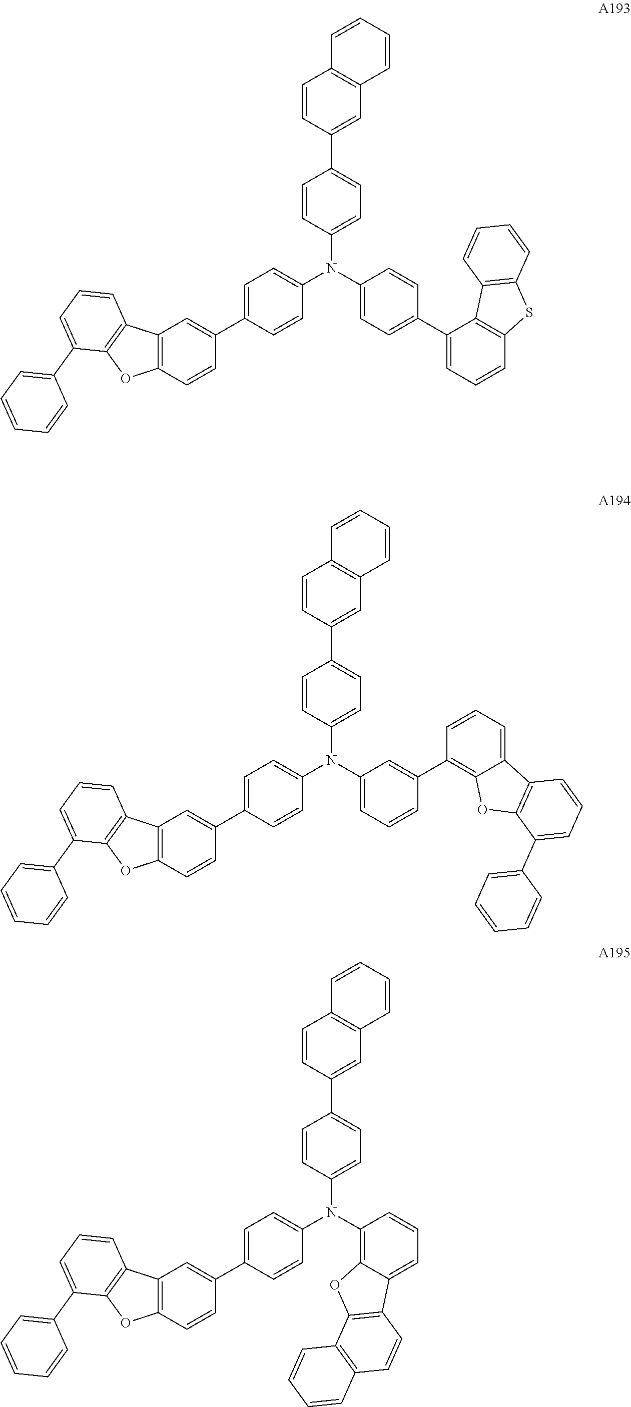 [SMILES: c1ccc(-c2cccc3c2oc2ccc(-c4ccc(N(c5ccc(-c6ccc7ccccc7c6)cc5)c5ccc(-c6cccc7sc8ccccc8c67)cc5)cc4)cc23)cc1 ; c1ccc(-c2cccc3c2oc2ccc(-c4ccc(N(c5ccc(-c6ccc7ccccc7c6)cc5)c5cccc(-c6cccc7c6oc6c(-c8ccccc8)cccc67)c5)cc4)cc23)cc1 ; c1ccc(-c2cccc3c2oc2ccc(-c4ccc(N(c5ccc(-c6ccc7ccccc7c6)cc5)c5cccc6c5oc5c7ccccc7ccc65)cc4)cc23)cc1]

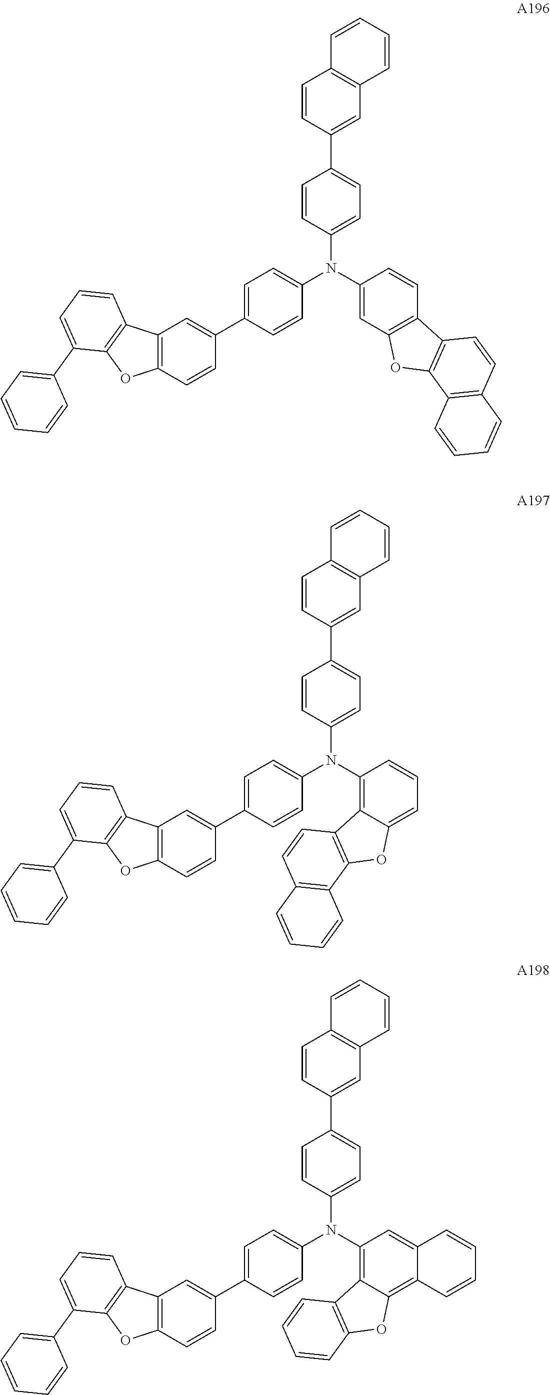 [SMILES: c1ccc(-c2cccc3c2oc2ccc(-c4ccc(N(c5ccc(-c6ccc7ccccc7c6)cc5)c5ccc6c(c5)oc5c7ccccc7ccc65)cc4)cc23)cc1 ; c1ccc(-c2cccc3c2oc2ccc(-c4ccc(N(c5ccc(-c6ccc7ccccc7c6)cc5)c5cccc6oc7c8ccccc8ccc7c56)cc4)cc23)cc1 ; c1ccc(-c2cccc3c2oc2ccc(-c4ccc(N(c5ccc(-c6ccc7ccccc7c6)cc5)c5cc6ccccc6c6oc7ccccc7c56)cc4)cc23)cc1]

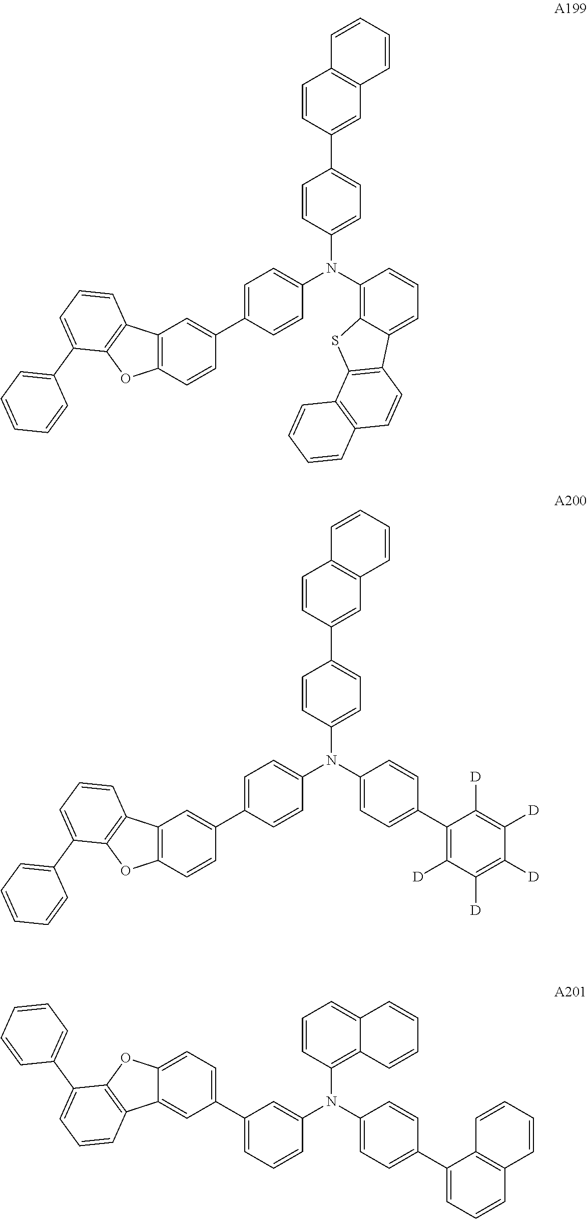 [SMILES: c1ccc(-c2cccc3c2oc2ccc(-c4ccc(N(c5ccc(-c6ccc7ccccc7c6)cc5)c5cccc6c5sc5c7ccccc7ccc65)cc4)cc23)cc1 ; [2H]c1c([2H])c([2H])c(-c2ccc(N(c3ccc(-c4ccc5ccccc5c4)cc3)c3ccc(-c4ccc5oc6c(-c7ccccc7)cccc6c5c4)cc3)cc2)c([2H])c1[2H] ; c1ccc(-c2cccc3c2oc2ccc(-c4cccc(N(c5ccc(-c6cccc7ccccc67)cc5)c5cccc6ccccc56)c4)cc23)cc1]

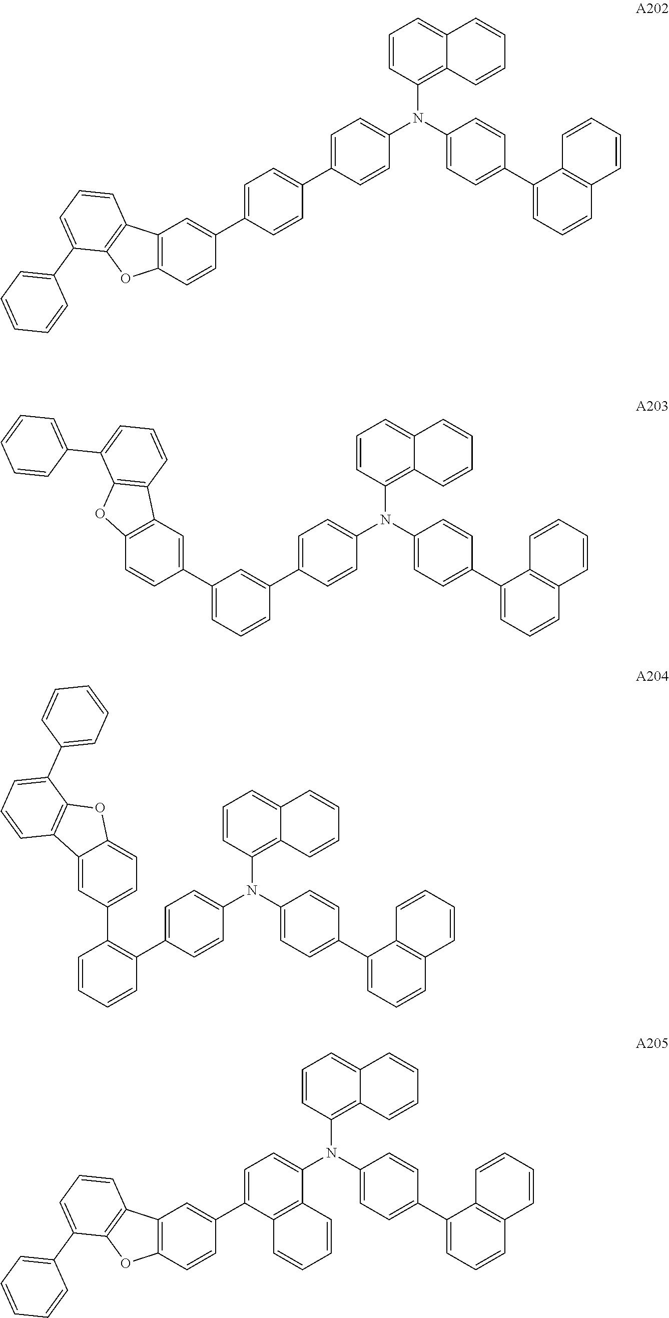

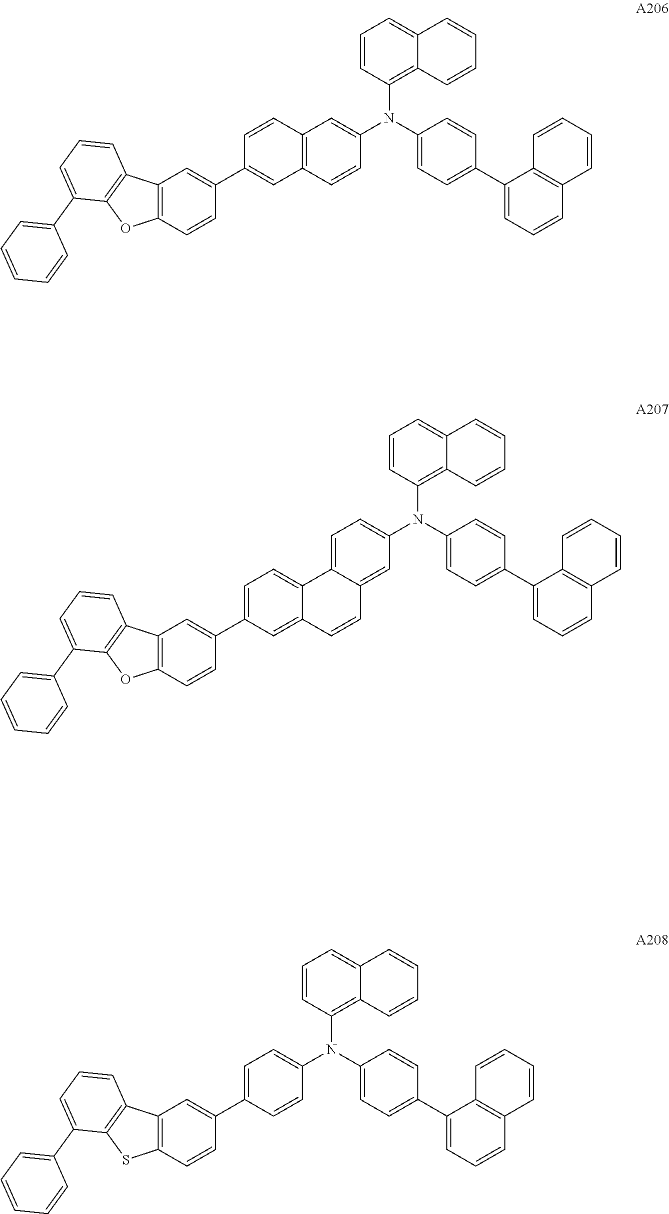 [SMILES: c1ccc(-c2cccc3c2oc2ccc(-c4ccc5cc(N(c6ccc(-c7cccc8ccccc78)cc6)c6cccc7ccccc67)ccc5c4)cc23)cc1 ; c1ccc(-c2cccc3c2oc2ccc(-c4ccc5c(ccc6cc(N(c7ccc(-c8cccc9ccccc89)cc7)c7cccc8ccccc78)ccc65)c4)cc23)cc1 ; c1ccc(-c2cccc3c2sc2ccc(-c4ccc(N(c5ccc(-c6cccc7ccccc67)cc5)c5cccc6ccccc56)cc4)cc23)cc1]

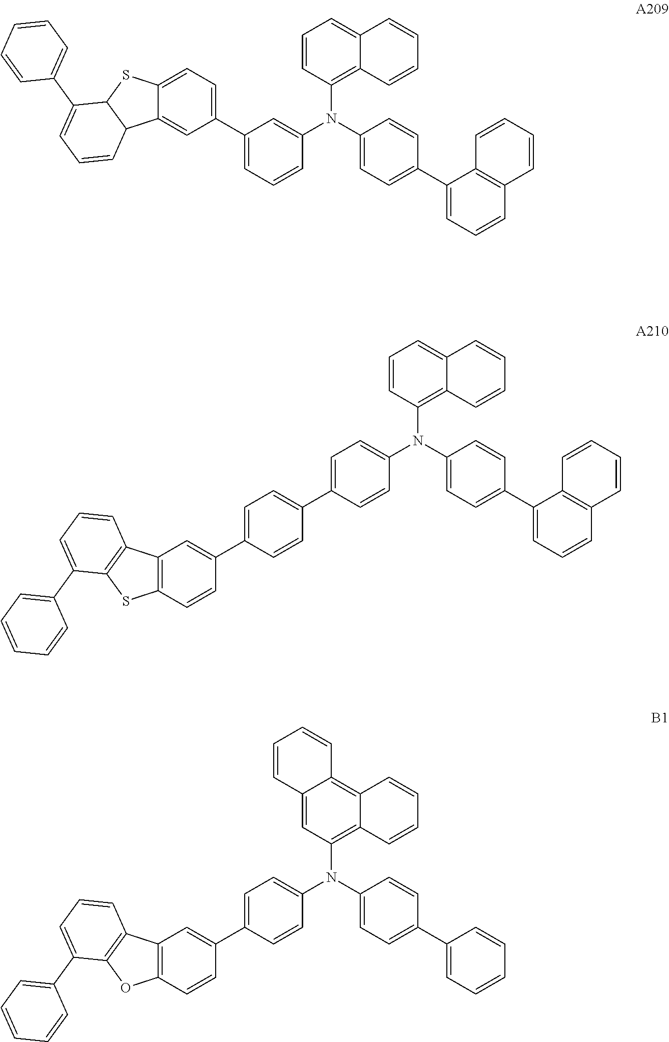 [SMILES: C1=CC2c3cc(-c4cccc(N(c5ccc(-c6cccc7ccccc67)cc5)c5cccc6ccccc56)c4)ccc3SC2C(c2ccccc2)=C1 ; c1ccc(-c2cccc3c2sc2ccc(-c4ccc(-c5ccc(N(c6ccc(-c7cccc8ccccc78)cc6)c6cccc7ccccc67)cc5)cc4)cc23)cc1 ; c1ccc(-c2ccc(N(c3ccc(-c4ccc5oc6c(-c7ccccc7)cccc6c5c4)cc3)c3cc4ccccc4c4ccccc34)cc2)cc1]

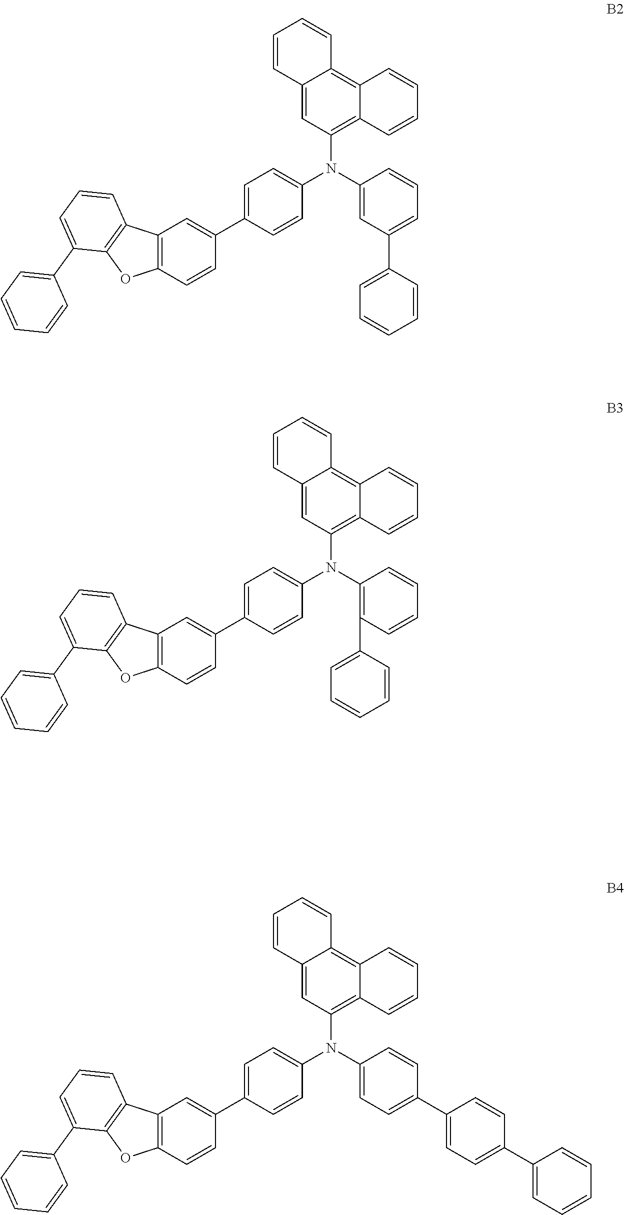 [SMILES: c1ccc(-c2cccc(N(c3ccc(-c4ccc5oc6c(-c7ccccc7)cccc6c5c4)cc3)c3cc4ccccc4c4ccccc34)c2)cc1 ; c1ccc(-c2ccccc2N(c2ccc(-c3ccc4oc5c(-c6ccccc6)cccc5c4c3)cc2)c2cc3ccccc3c3ccccc23)cc1 ; c1ccc(-c2ccc(-c3ccc(N(c4ccc(-c5ccc6oc7c(-c8ccccc8)cccc7c6c5)cc4)c4cc5ccccc5c5ccccc45)cc3)cc2)cc1]

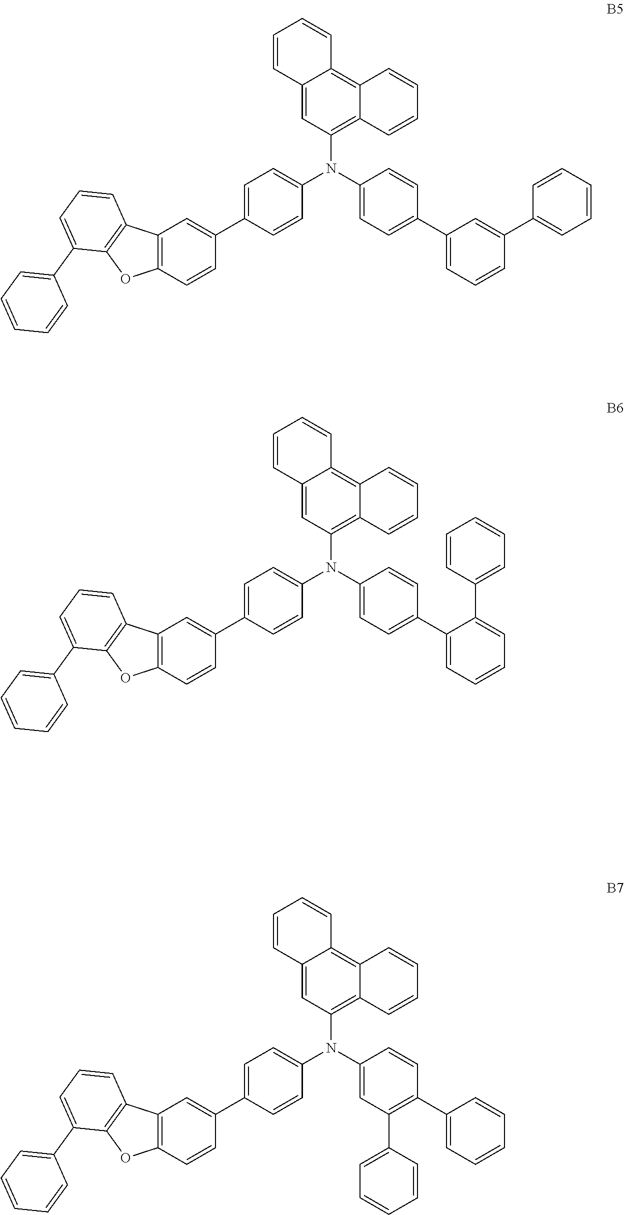 [SMILES: c1ccc(-c2cccc(-c3ccc(N(c4ccc(-c5ccc6oc7c(-c8ccccc8)cccc7c6c5)cc4)c4cc5ccccc5c5ccccc45)cc3)c2)cc1 ; c1ccc(-c2ccccc2-c2ccc(N(c3ccc(-c4ccc5oc6c(-c7ccccc7)cccc6c5c4)cc3)c3cc4ccccc4c4ccccc34)cc2)cc1 ; c1ccc(-c2ccc(N(c3ccc(-c4ccc5oc6c(-c7ccccc7)cccc6c5c4)cc3)c3cc4ccccc4c4ccccc34)cc2-c2ccccc2)cc1]

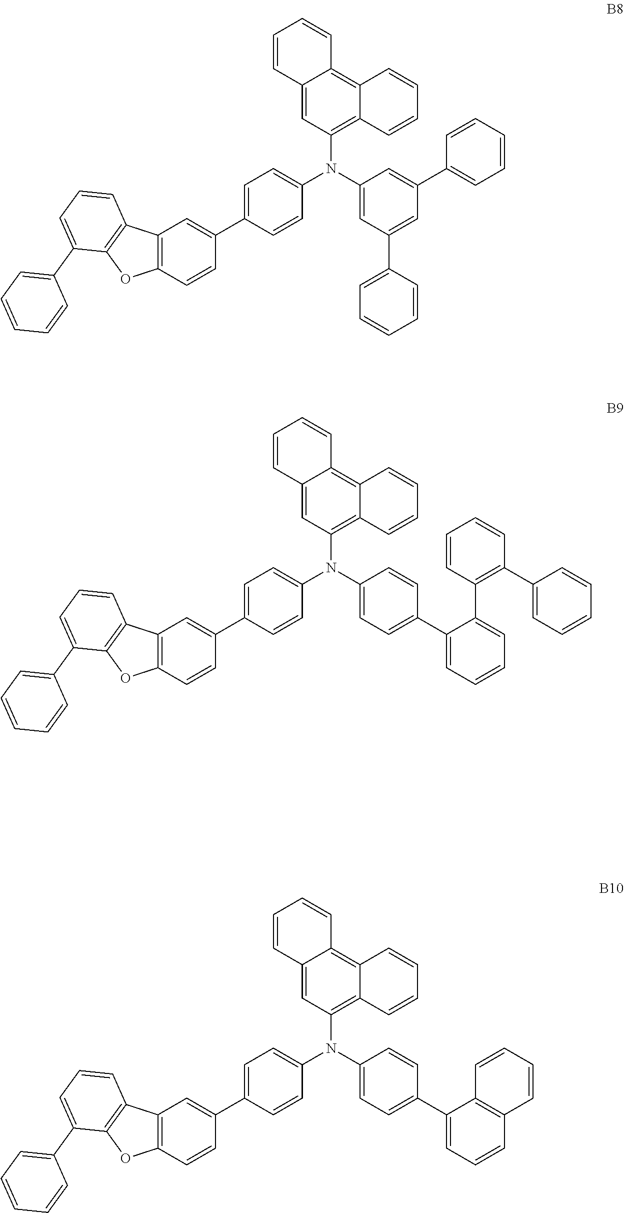 [SMILES: c1ccc(-c2cc(-c3ccccc3)cc(N(c3ccc(-c4ccc5oc6c(-c7ccccc7)cccc6c5c4)cc3)c3cc4ccccc4c4ccccc34)c2)cc1 ; c1ccc(-c2ccccc2-c2ccccc2-c2ccc(N(c3ccc(-c4ccc5oc6c(-c7ccccc7)cccc6c5c4)cc3)c3cc4ccccc4c4ccccc34)cc2)cc1 ; c1ccc(-c2cccc3c2oc2ccc(-c4ccc(N(c5ccc(-c6cccc7ccccc67)cc5)c5cc6ccccc6c6ccccc56)cc4)cc23)cc1]

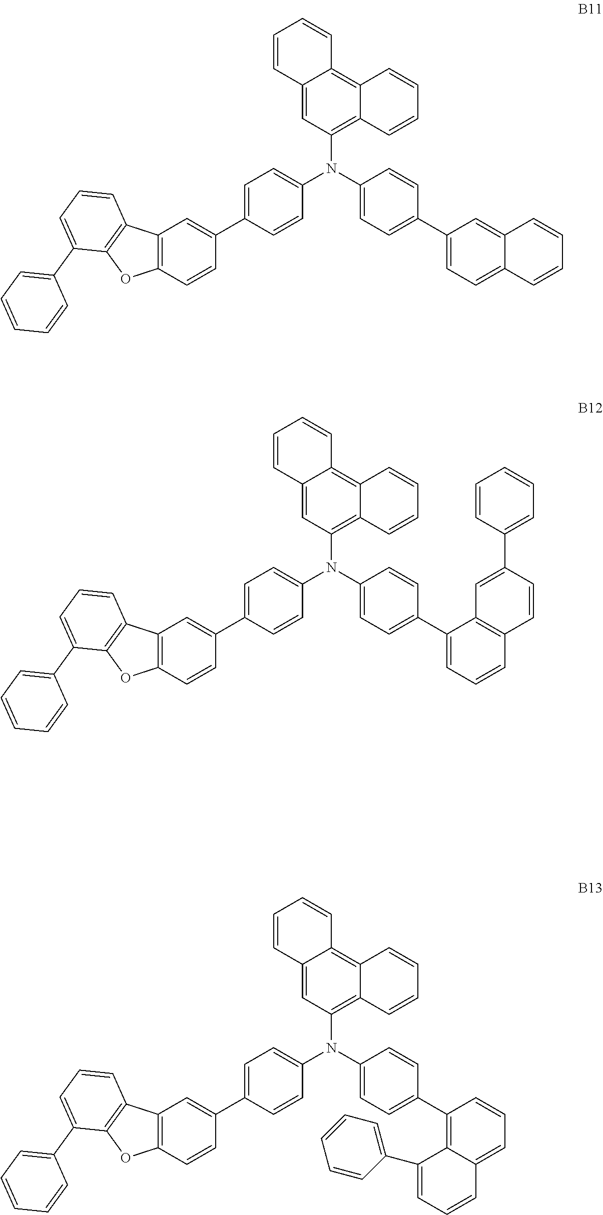 [SMILES: c1ccc(-c2cccc3c2oc2ccc(-c4ccc(N(c5ccc(-c6ccc7ccccc7c6)cc5)c5cc6ccccc6c6ccccc56)cc4)cc23)cc1 ; c1ccc(-c2ccc3cccc(-c4ccc(N(c5ccc(-c6ccc7oc8c(-c9ccccc9)cccc8c7c6)cc5)c5cc6ccccc6c6ccccc56)cc4)c3c2)cc1 ; c1ccc(-c2cccc3c2oc2ccc(-c4ccc(N(c5ccc(-c6cccc7cccc(-c8ccccc8)c67)cc5)c5cc6ccccc6c6ccccc56)cc4)cc23)cc1]

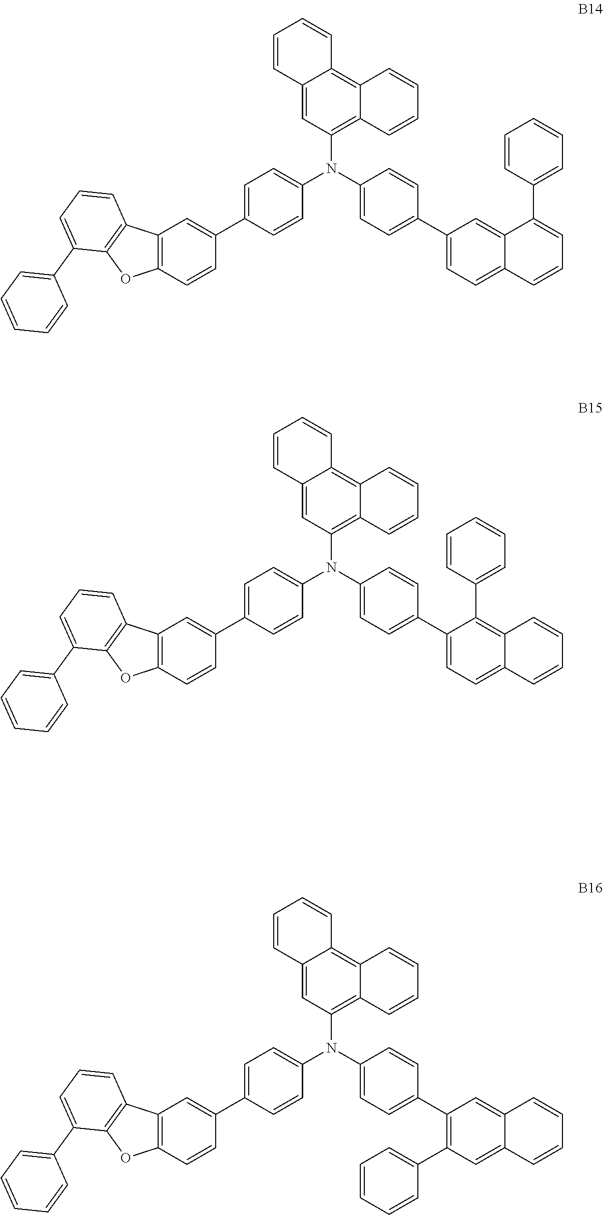 [SMILES: c1ccc(-c2cccc3ccc(-c4ccc(N(c5ccc(-c6ccc7oc8c(-c9ccccc9)cccc8c7c6)cc5)c5cc6ccccc6c6ccccc56)cc4)cc23)cc1 ; c1ccc(-c2c(-c3ccc(N(c4ccc(-c5ccc6oc7c(-c8ccccc8)cccc7c6c5)cc4)c4cc5ccccc5c5ccccc45)cc3)ccc3ccccc23)cc1 ; c1ccc(-c2cc3ccccc3cc2-c2ccc(N(c3ccc(-c4ccc5oc6c(-c7ccccc7)cccc6c5c4)cc3)c3cc4ccccc4c4ccccc34)cc2)cc1]

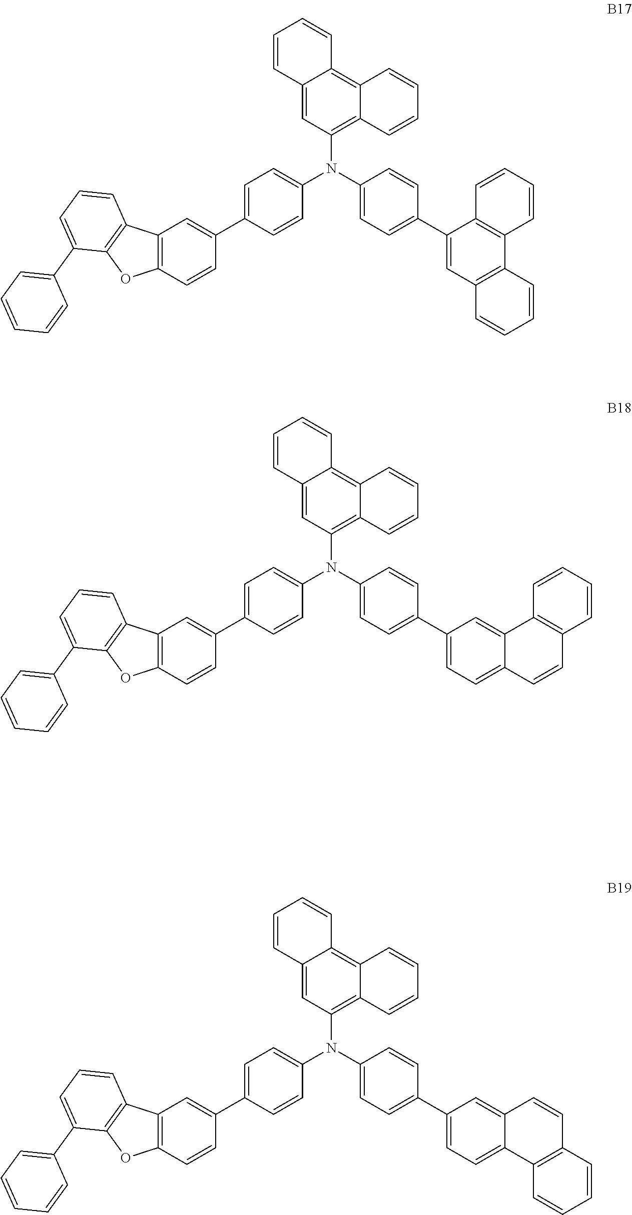 [SMILES: c1ccc(-c2cccc3c2oc2ccc(-c4ccc(N(c5ccc(-c6cc7ccccc7c7ccccc67)cc5)c5cc6ccccc6c6ccccc56)cc4)cc23)cc1 ; c1ccc(-c2cccc3c2oc2ccc(-c4ccc(N(c5ccc(-c6ccc7ccc8ccccc8c7c6)cc5)c5cc6ccccc6c6ccccc56)cc4)cc23)cc1 ; c1ccc(-c2cccc3c2oc2ccc(-c4ccc(N(c5ccc(-c6ccc7c(ccc8ccccc87)c6)cc5)c5cc6ccccc6c6ccccc56)cc4)cc23)cc1]

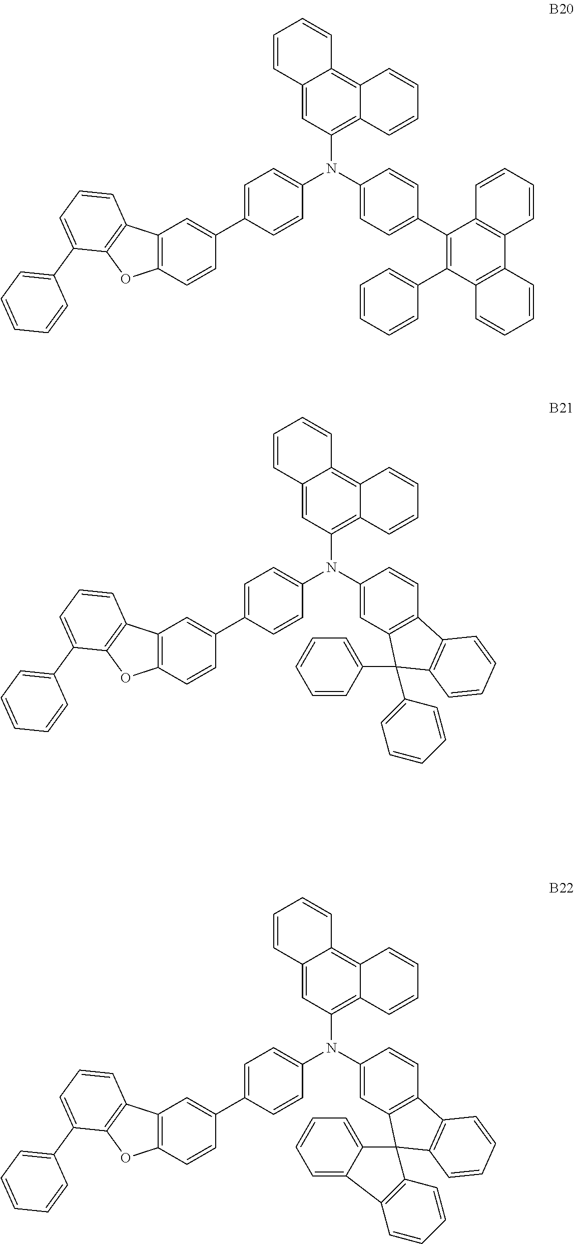 [SMILES: c1ccc(-c2c(-c3ccc(N(c4ccc(-c5ccc6oc7c(-c8ccccc8)cccc7c6c5)cc4)c4cc5ccccc5c5ccccc45)cc3)c3ccccc3c3ccccc23)cc1 ; c1ccc(-c2cccc3c2oc2ccc(-c4ccc(N(c5ccc6c(c5)C(c5ccccc5)(c5ccccc5)c5ccccc5-6)c5cc6ccccc6c6ccccc56)cc4)cc23)cc1 ; c1ccc(-c2cccc3c2oc2ccc(-c4ccc(N(c5ccc6c(c5)C5(c7ccccc7-c7ccccc75)c5ccccc5-6)c5cc6ccccc6c6ccccc56)cc4)cc23)cc1]

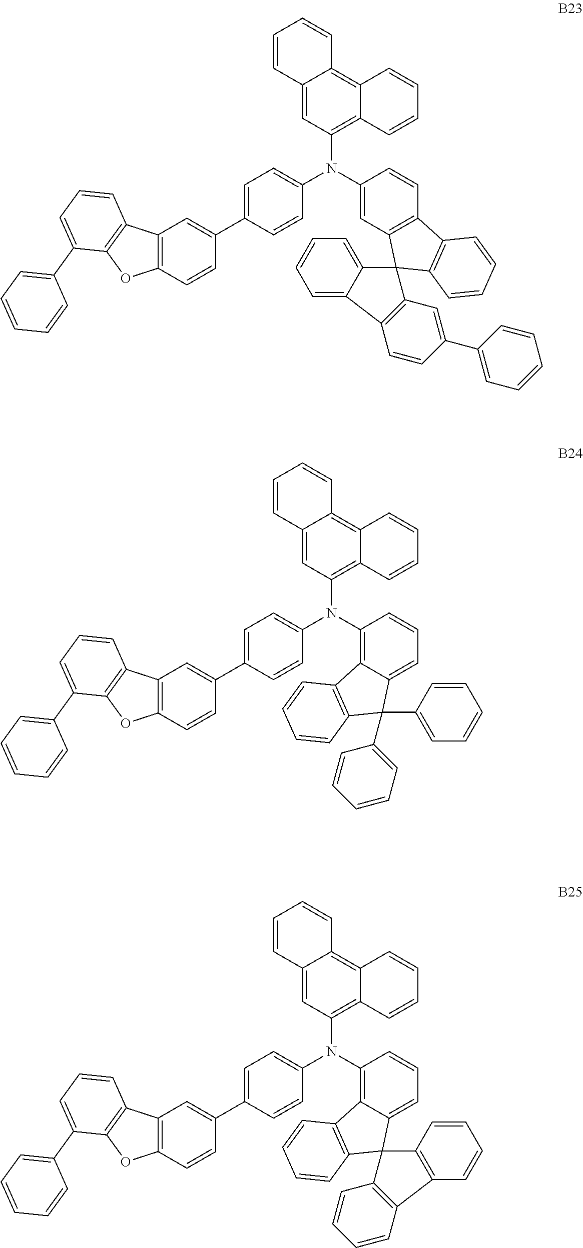 [SMILES: c1ccc(-c2ccc3c(c2)C2(c4ccccc4-3)c3ccccc3-c3ccc(N(c4ccc(-c5ccc6oc7c(-c8ccccc8)cccc7c6c5)cc4)c4cc5ccccc5c5ccccc45)cc32)cc1 ; c1ccc(-c2cccc3c2oc2ccc(-c4ccc(N(c5cccc6c5-c5ccccc5C6(c5ccccc5)c5ccccc5)c5cc6ccccc6c6ccccc56)cc4)cc23)cc1 ; c1ccc(-c2cccc3c2oc2ccc(-c4ccc(N(c5cccc6c5-c5ccccc5C65c6ccccc6-c6ccccc65)c5cc6ccccc6c6ccccc56)cc4)cc23)cc1]

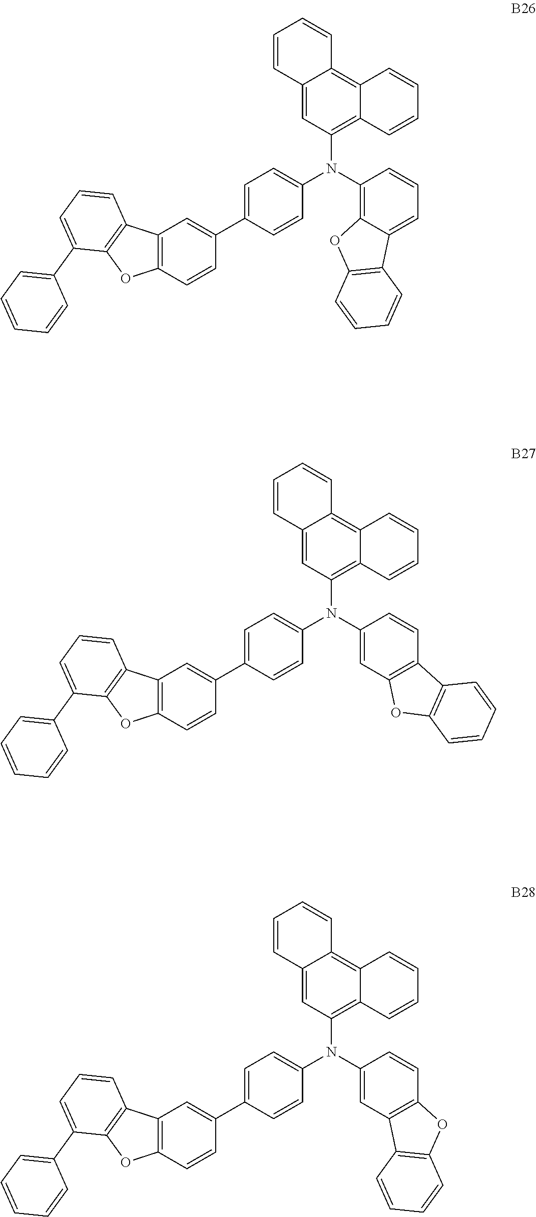 [SMILES: c1ccc(-c2cccc3c2oc2ccc(-c4ccc(N(c5cc6ccccc6c6ccccc56)c5cccc6c5oc5ccccc56)cc4)cc23)cc1 ; c1ccc(-c2cccc3c2oc2ccc(-c4ccc(N(c5ccc6c(c5)oc5ccccc56)c5cc6ccccc6c6ccccc56)cc4)cc23)cc1 ; c1ccc(-c2cccc3c2oc2ccc(-c4ccc(N(c5ccc6oc7ccccc7c6c5)c5cc6ccccc6c6ccccc56)cc4)cc23)cc1]

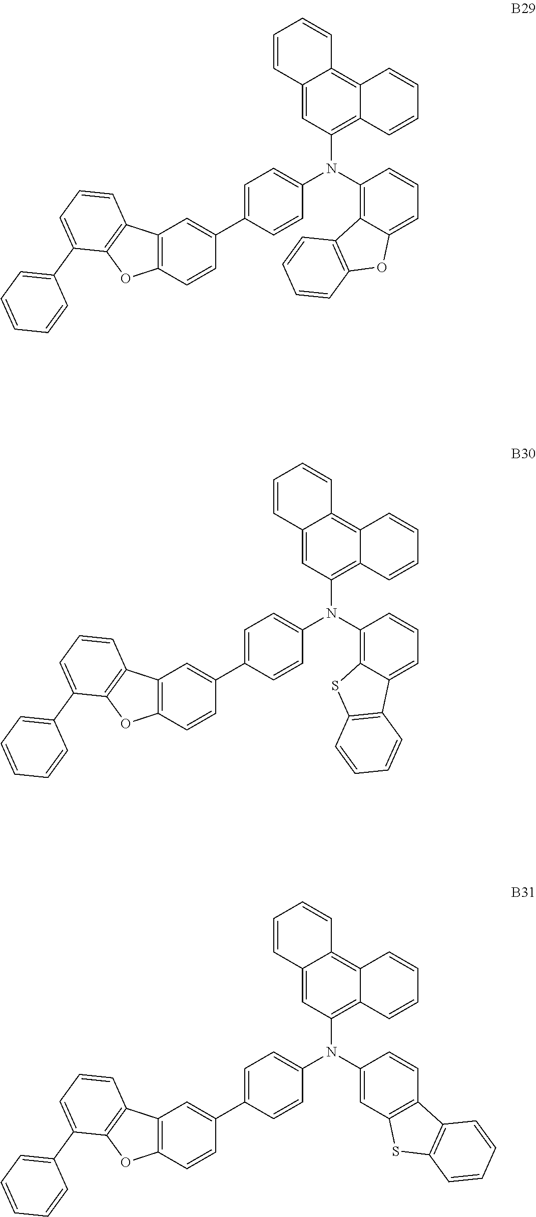 [SMILES: c1ccc(-c2cccc3c2oc2ccc(-c4ccc(N(c5cc6ccccc6c6ccccc56)c5cccc6oc7ccccc7c56)cc4)cc23)cc1 ; c1ccc(-c2cccc3c2oc2ccc(-c4ccc(N(c5cc6ccccc6c6ccccc56)c5cccc6c5sc5ccccc56)cc4)cc23)cc1 ; c1ccc(-c2cccc3c2oc2ccc(-c4ccc(N(c5ccc6c(c5)sc5ccccc56)c5cc6ccccc6c6ccccc56)cc4)cc23)cc1]

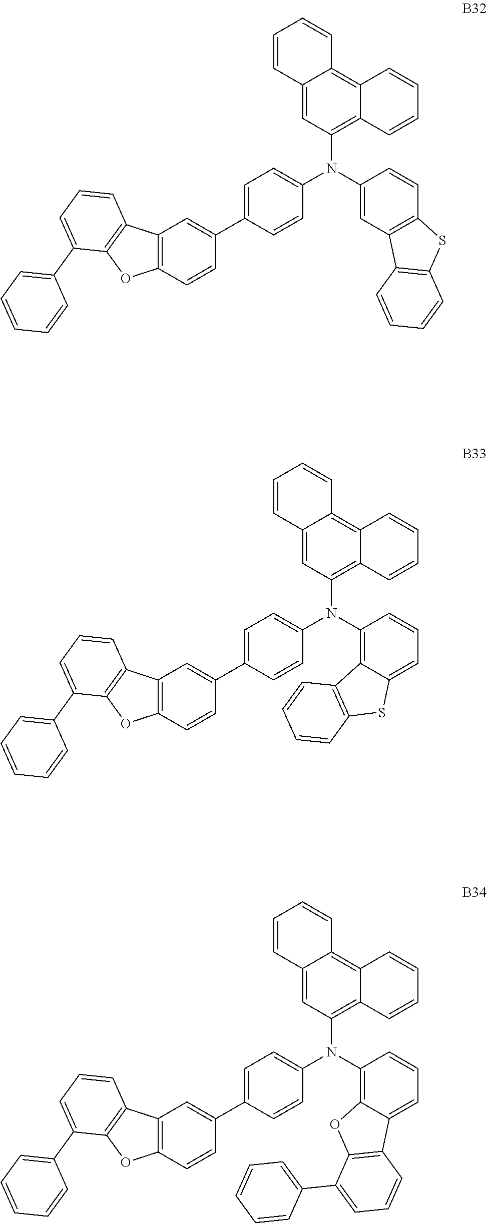 [SMILES: c1ccc(-c2cccc3c2oc2ccc(-c4ccc(N(c5ccc6sc7ccccc7c6c5)c5cc6ccccc6c6ccccc56)cc4)cc23)cc1 ; c1ccc(-c2cccc3c2oc2ccc(-c4ccc(N(c5cc6ccccc6c6ccccc56)c5cccc6sc7ccccc7c56)cc4)cc23)cc1 ; c1ccc(-c2cccc3c2oc2ccc(-c4ccc(N(c5cc6ccccc6c6ccccc56)c5cccc6c5oc5c(-c7ccccc7)cccc56)cc4)cc23)cc1]

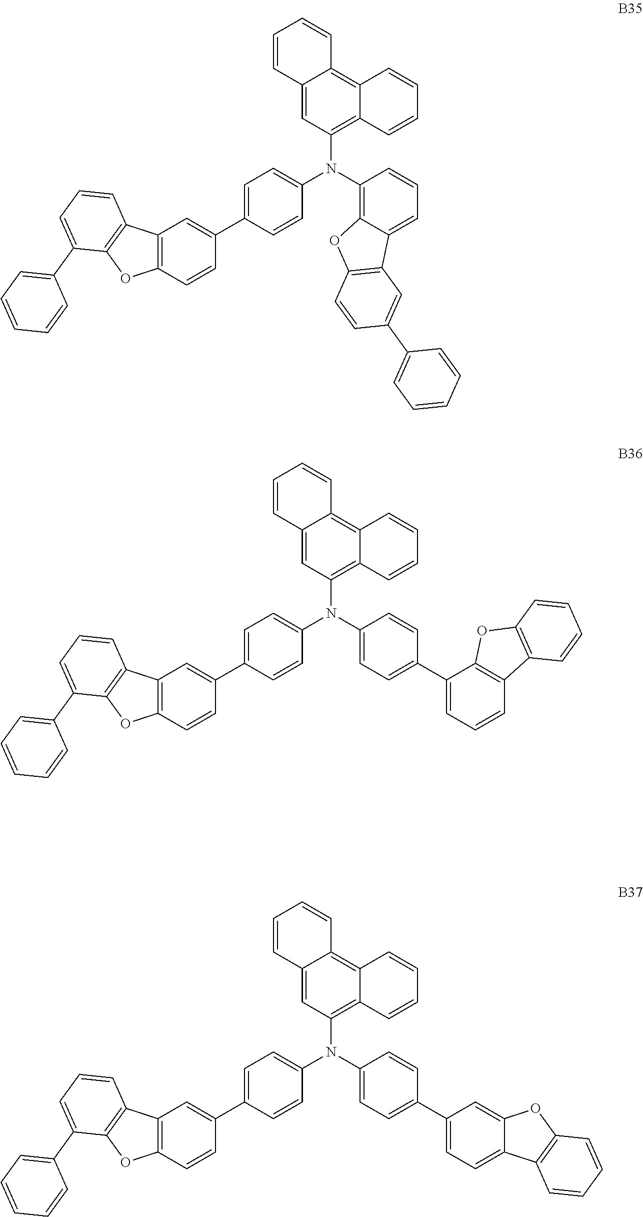 [SMILES: c1ccc(-c2ccc3oc4c(N(c5ccc(-c6ccc7oc8c(-c9ccccc9)cccc8c7c6)cc5)c5cc6ccccc6c6ccccc56)cccc4c3c2)cc1 ; c1ccc(-c2cccc3c2oc2ccc(-c4ccc(N(c5ccc(-c6cccc7c6oc6ccccc67)cc5)c5cc6ccccc6c6ccccc56)cc4)cc23)cc1 ; c1ccc(-c2cccc3c2oc2ccc(-c4ccc(N(c5ccc(-c6ccc7c(c6)oc6ccccc67)cc5)c5cc6ccccc6c6ccccc56)cc4)cc23)cc1]

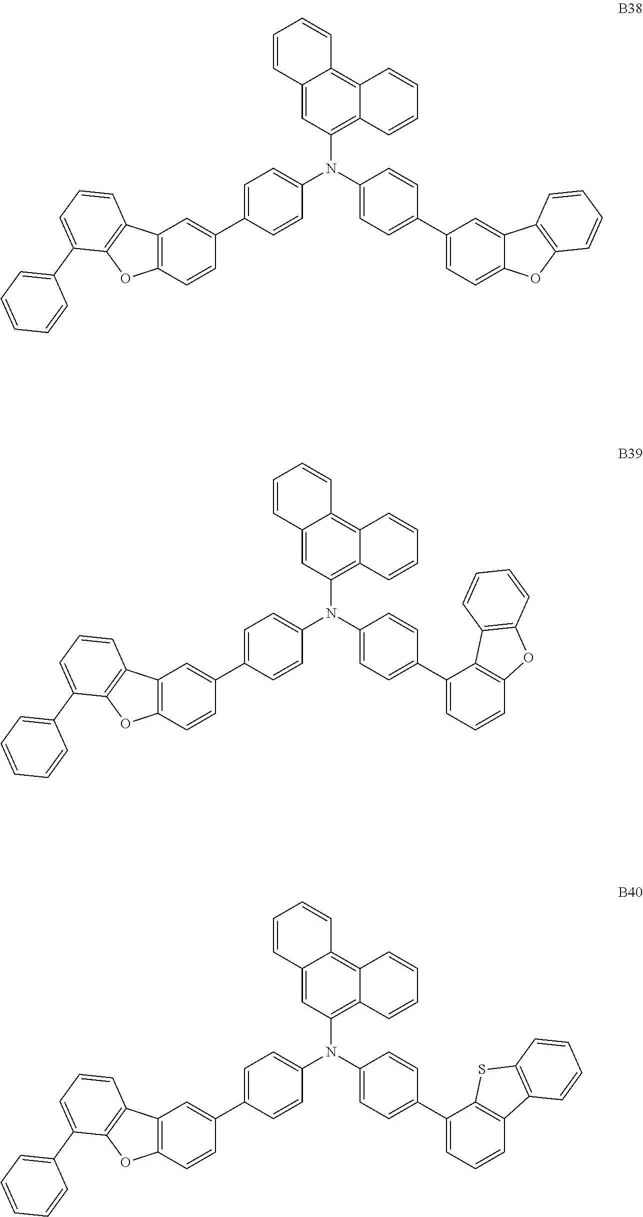 [SMILES: c1ccc(-c2cccc3c2oc2ccc(-c4ccc(N(c5ccc(-c6ccc7oc8ccccc8c7c6)cc5)c5cc6ccccc6c6ccccc56)cc4)cc23)cc1 ; c1ccc(-c2cccc3c2oc2ccc(-c4ccc(N(c5ccc(-c6cccc7oc8ccccc8c67)cc5)c5cc6ccccc6c6ccccc56)cc4)cc23)cc1 ; c1ccc(-c2cccc3c2oc2ccc(-c4ccc(N(c5ccc(-c6cccc7c6sc6ccccc67)cc5)c5cc6ccccc6c6ccccc56)cc4)cc23)cc1]

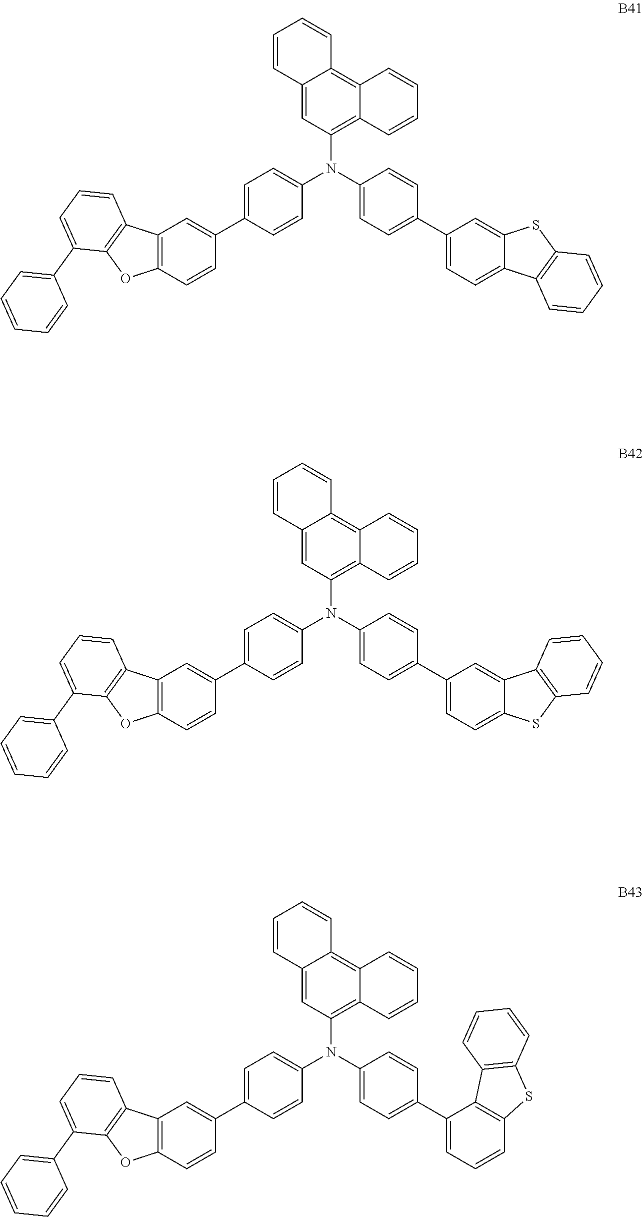 [SMILES: c1ccc(-c2cccc3c2oc2ccc(-c4ccc(N(c5ccc(-c6ccc7c(c6)sc6ccccc67)cc5)c5cc6ccccc6c6ccccc56)cc4)cc23)cc1 ; c1ccc(-c2cccc3c2oc2ccc(-c4ccc(N(c5ccc(-c6ccc7sc8ccccc8c7c6)cc5)c5cc6ccccc6c6ccccc56)cc4)cc23)cc1 ; c1ccc(-c2cccc3c2oc2ccc(-c4ccc(N(c5ccc(-c6cccc7sc8ccccc8c67)cc5)c5cc6ccccc6c6ccccc56)cc4)cc23)cc1]

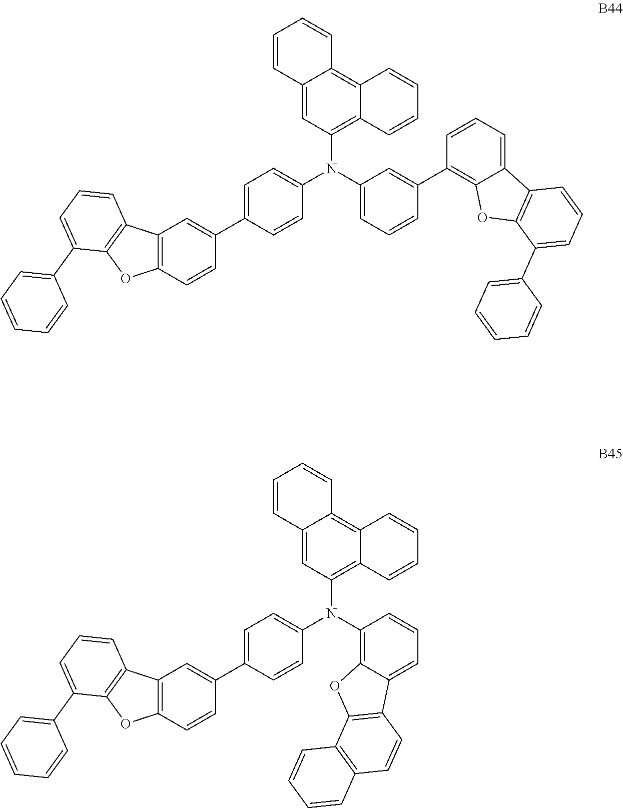 [SMILES: c1ccc(-c2cccc3c2oc2ccc(-c4ccc(N(c5cccc(-c6cccc7c6oc6c(-c8ccccc8)cccc67)c5)c5cc6ccccc6c6ccccc56)cc4)cc23)cc1 ; c1ccc(-c2cccc3c2oc2ccc(-c4ccc(N(c5cc6ccccc6c6ccccc56)c5cccc6c5oc5c7ccccc7ccc65)cc4)cc23)cc1]

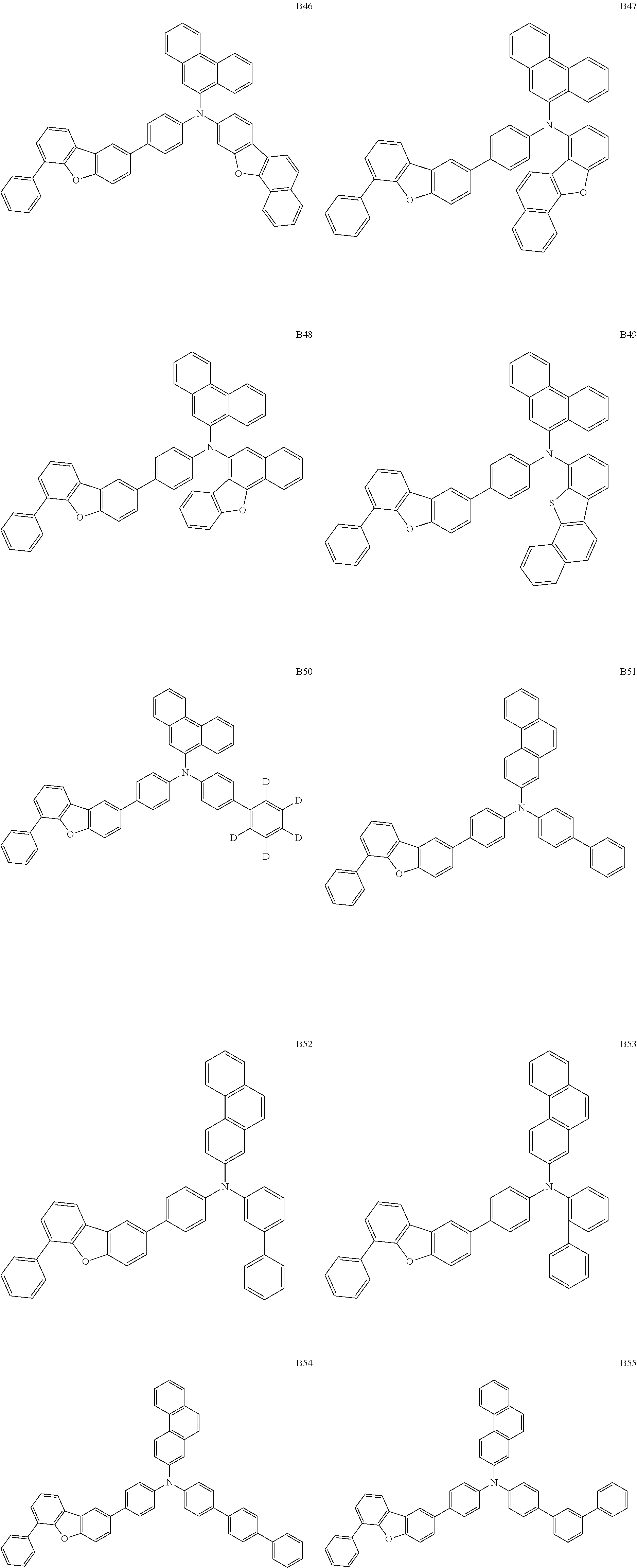

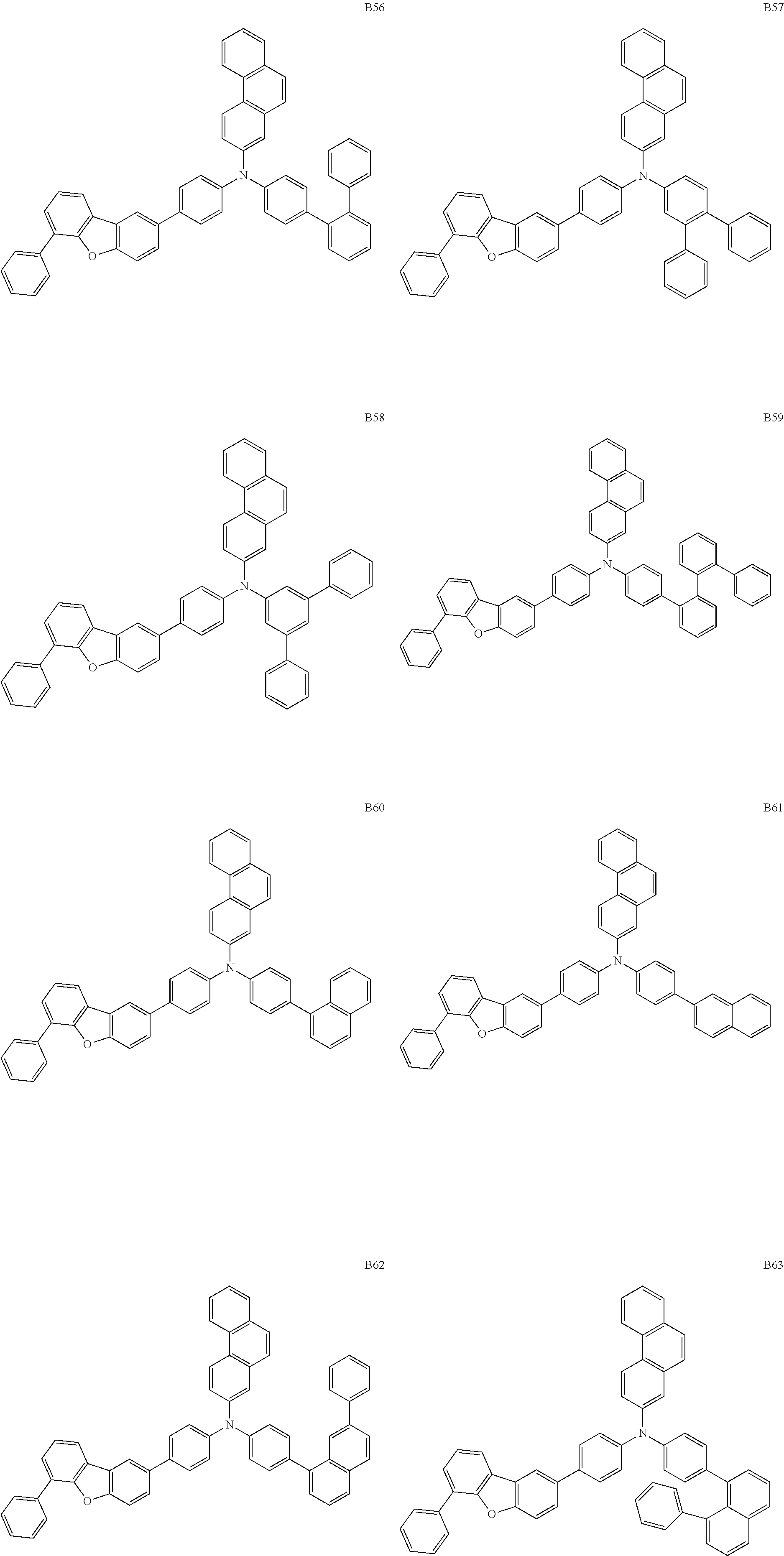

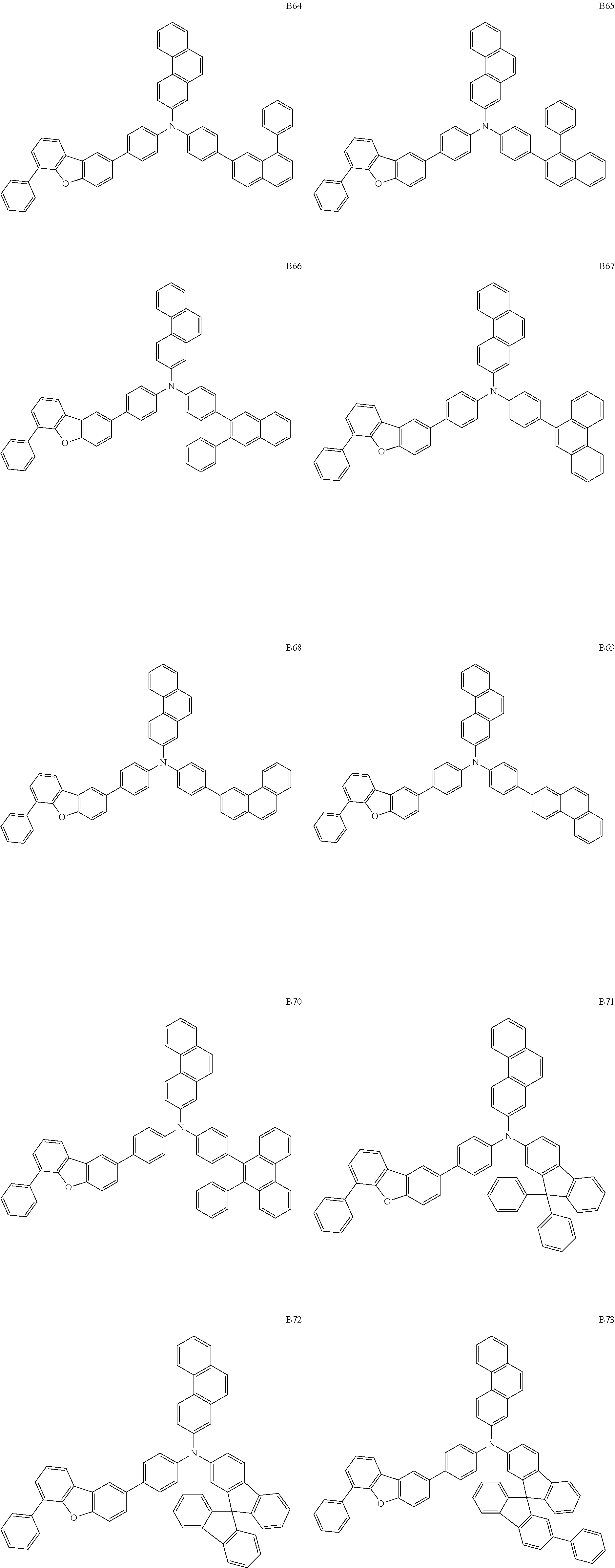

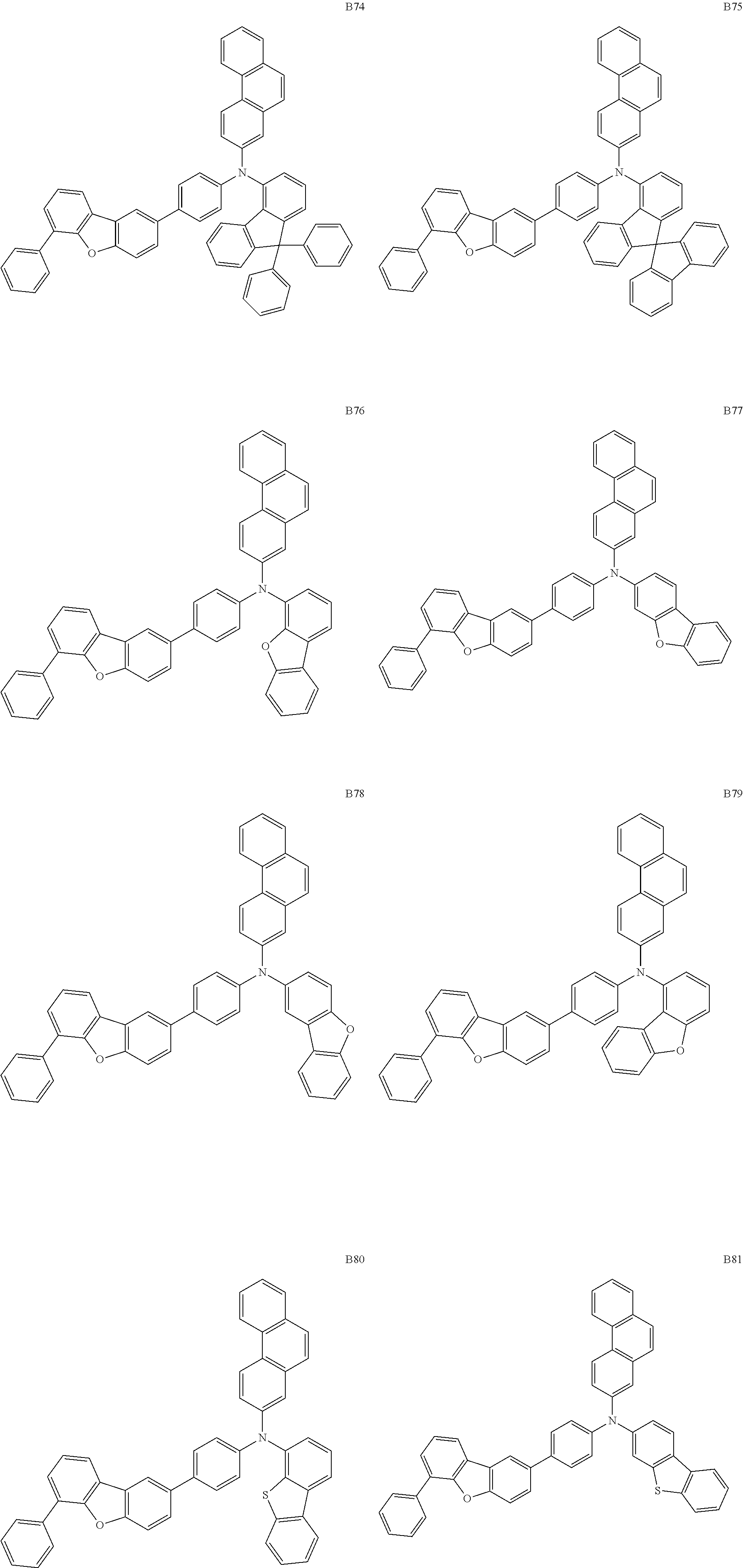

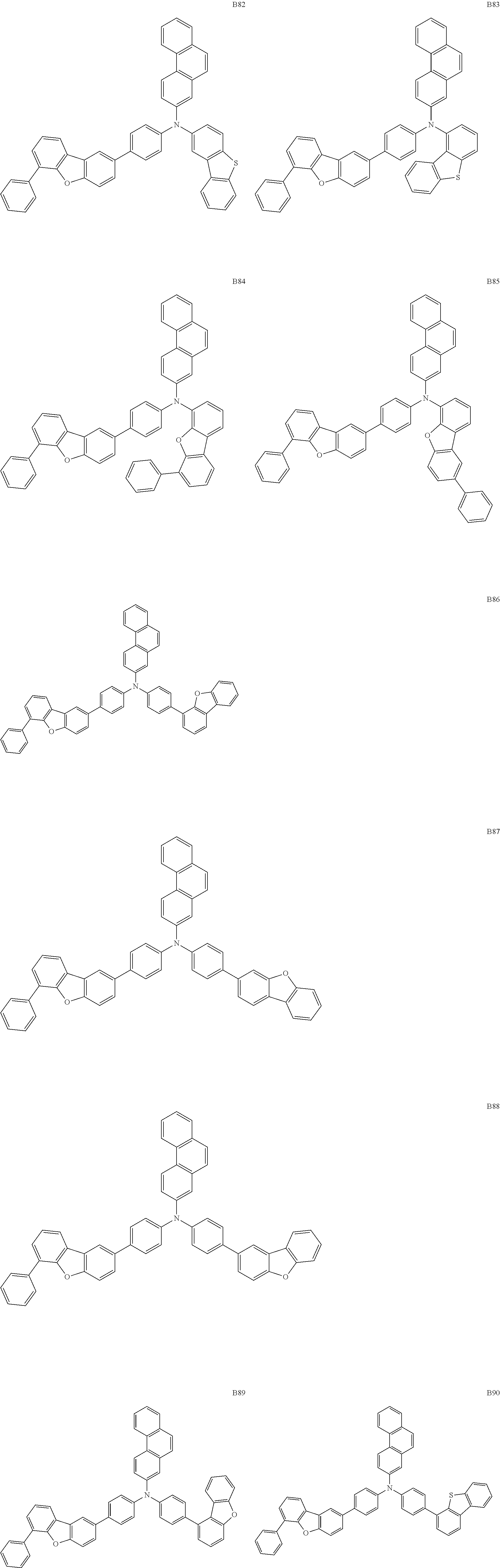

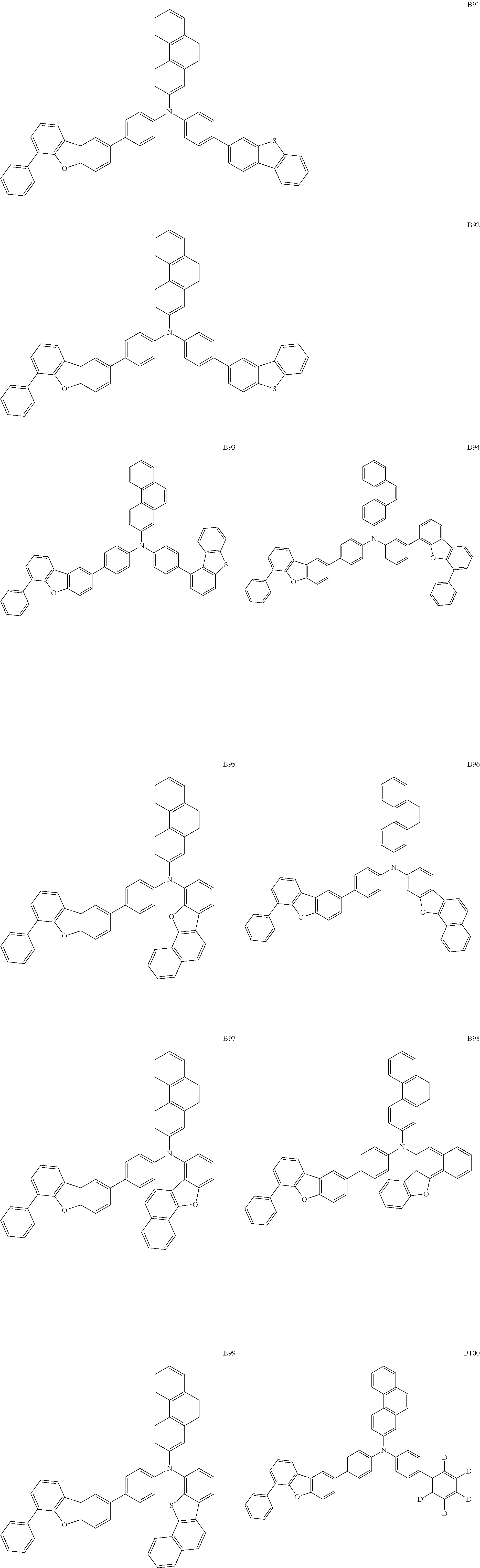

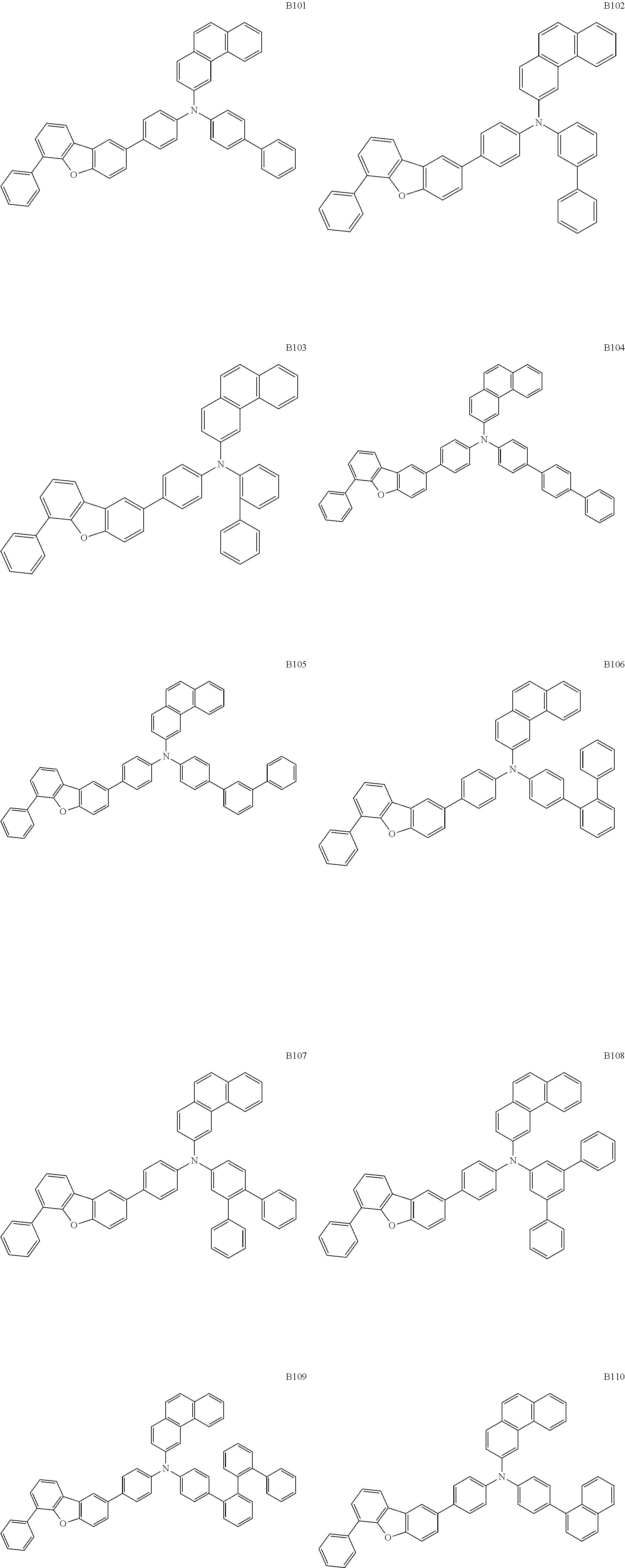

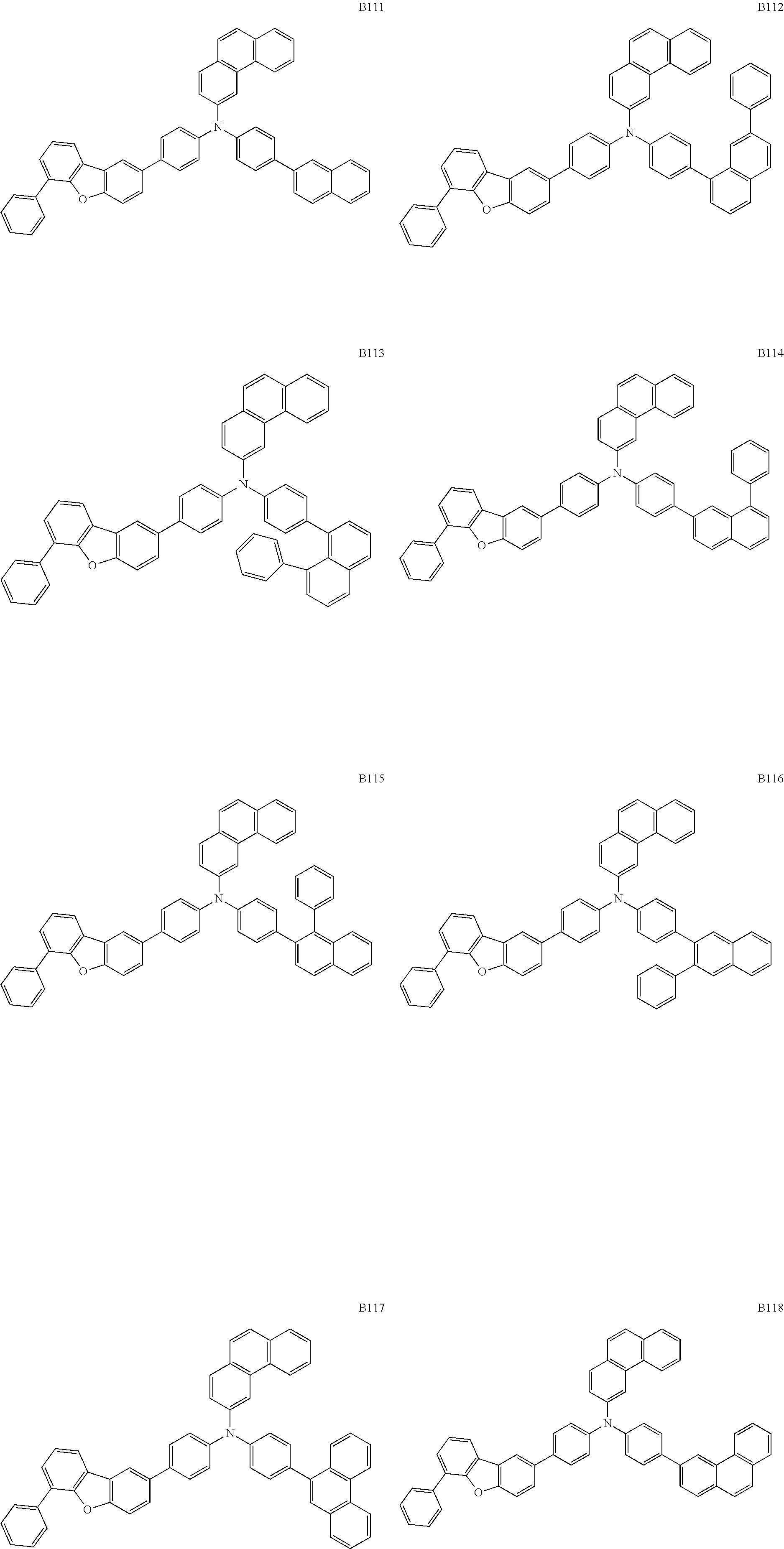

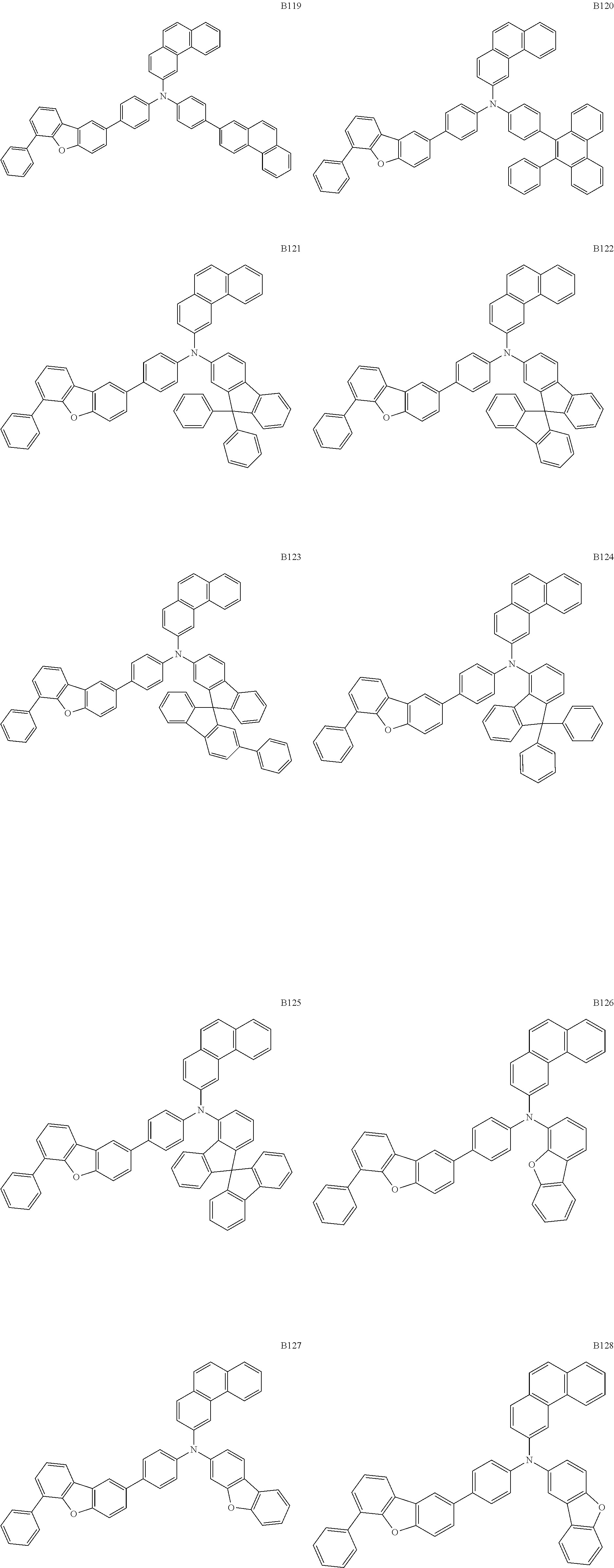

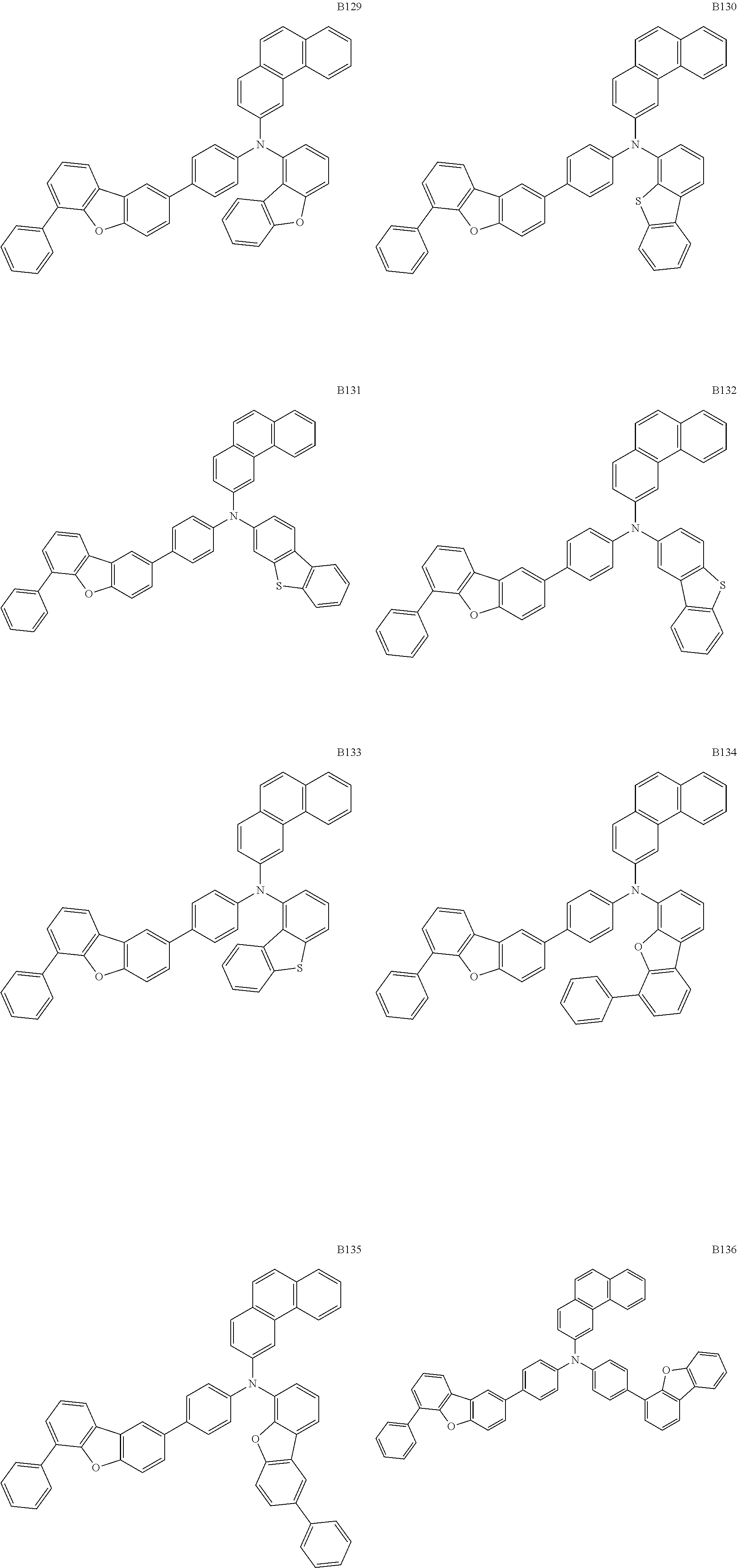

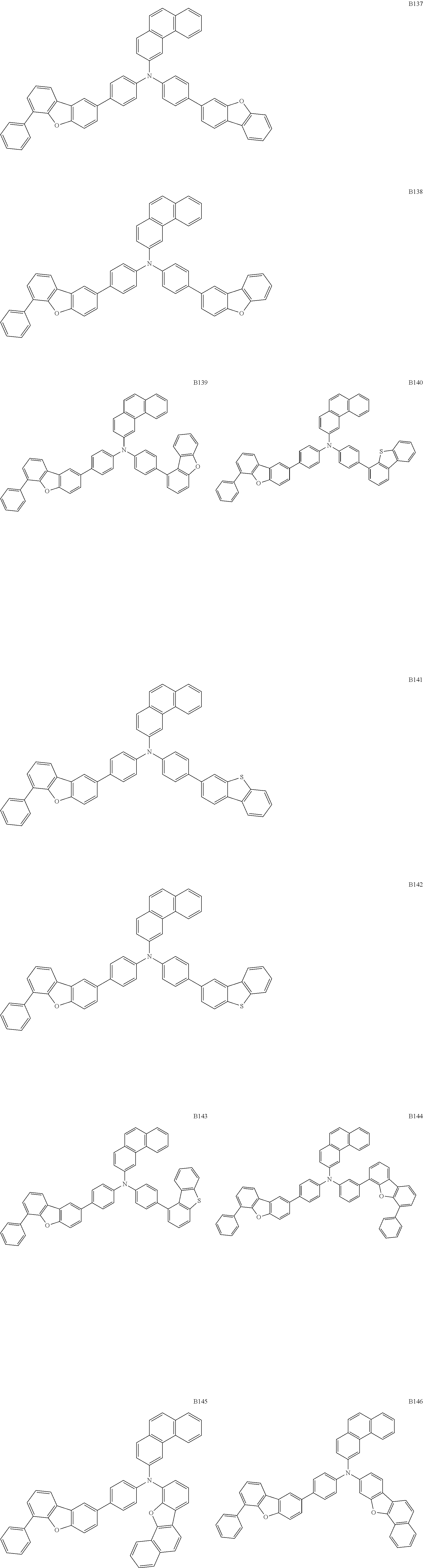

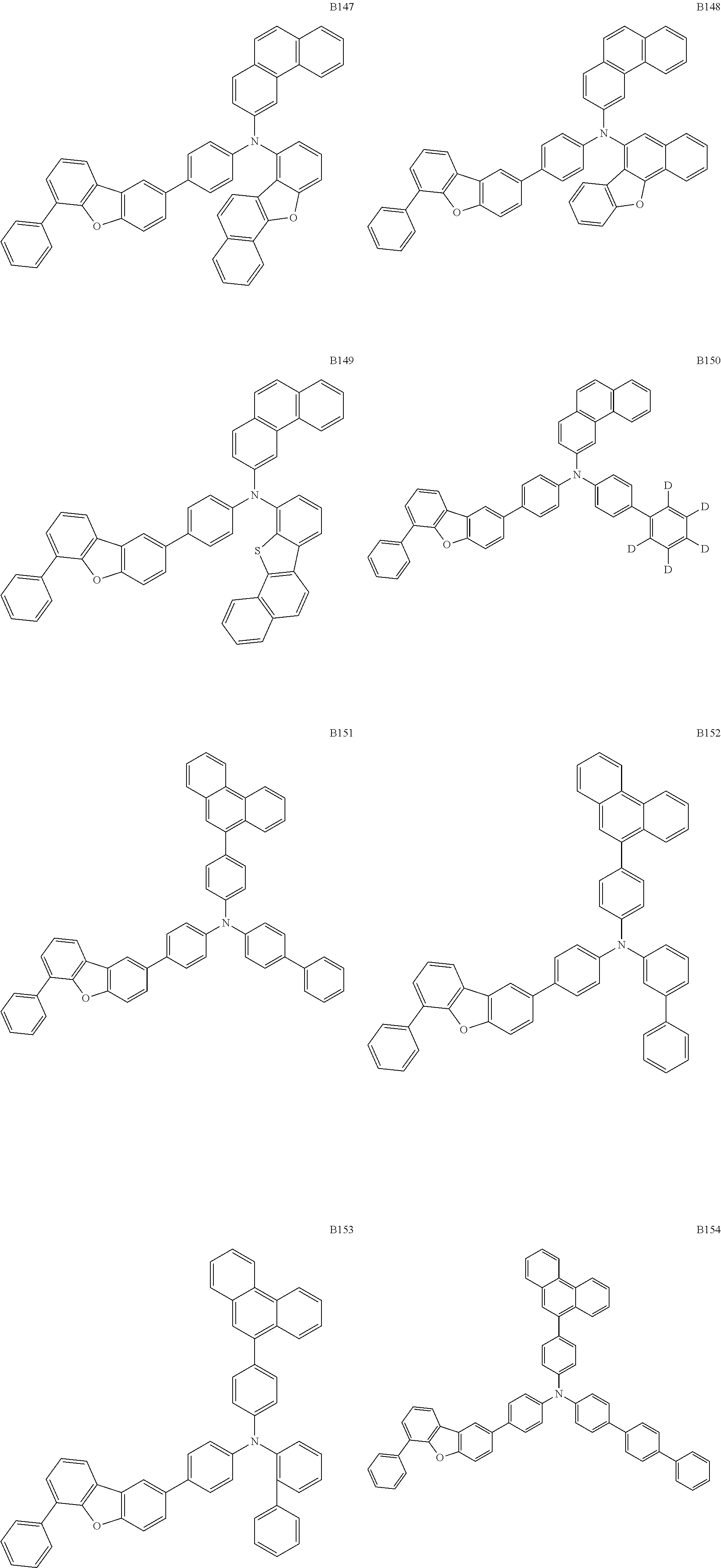

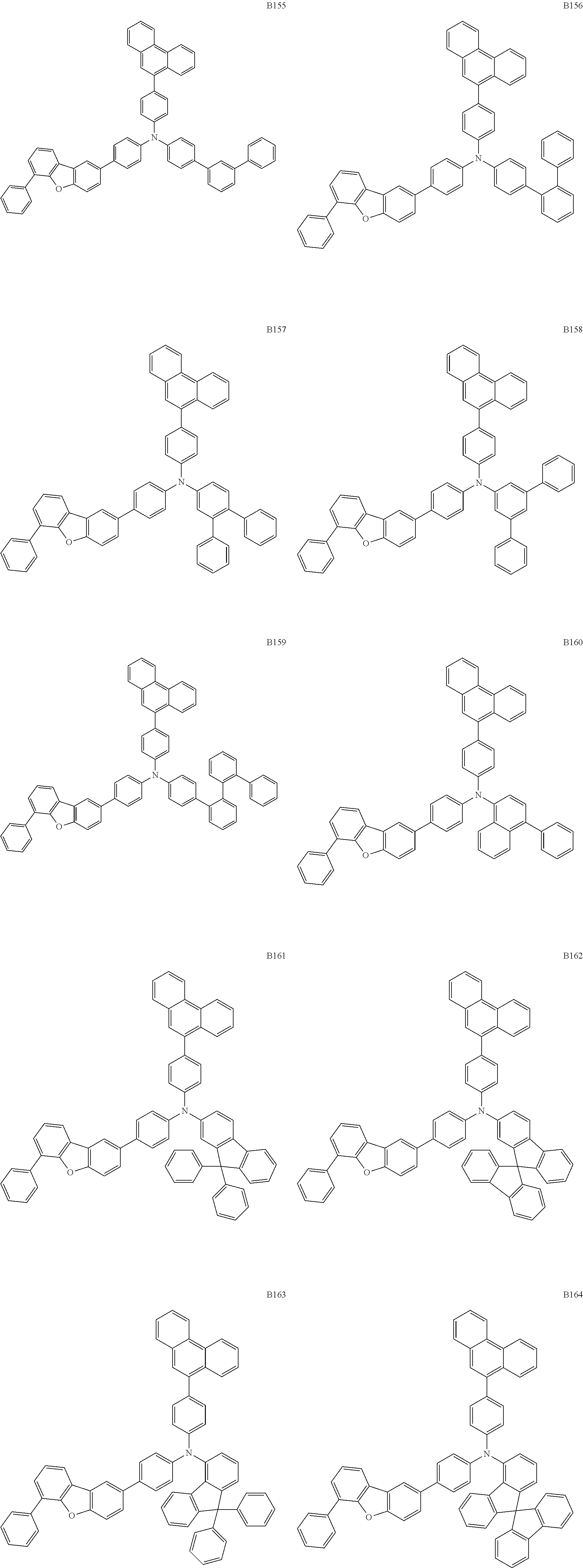

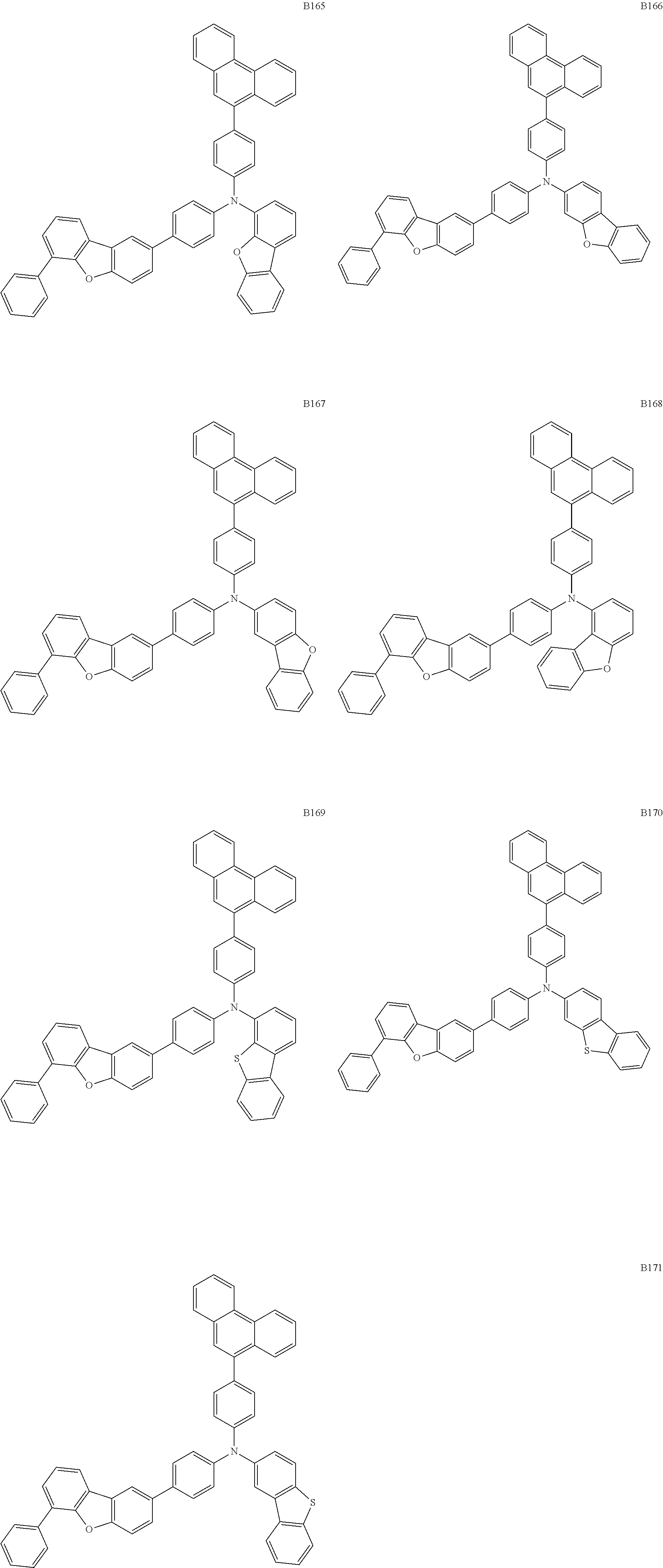

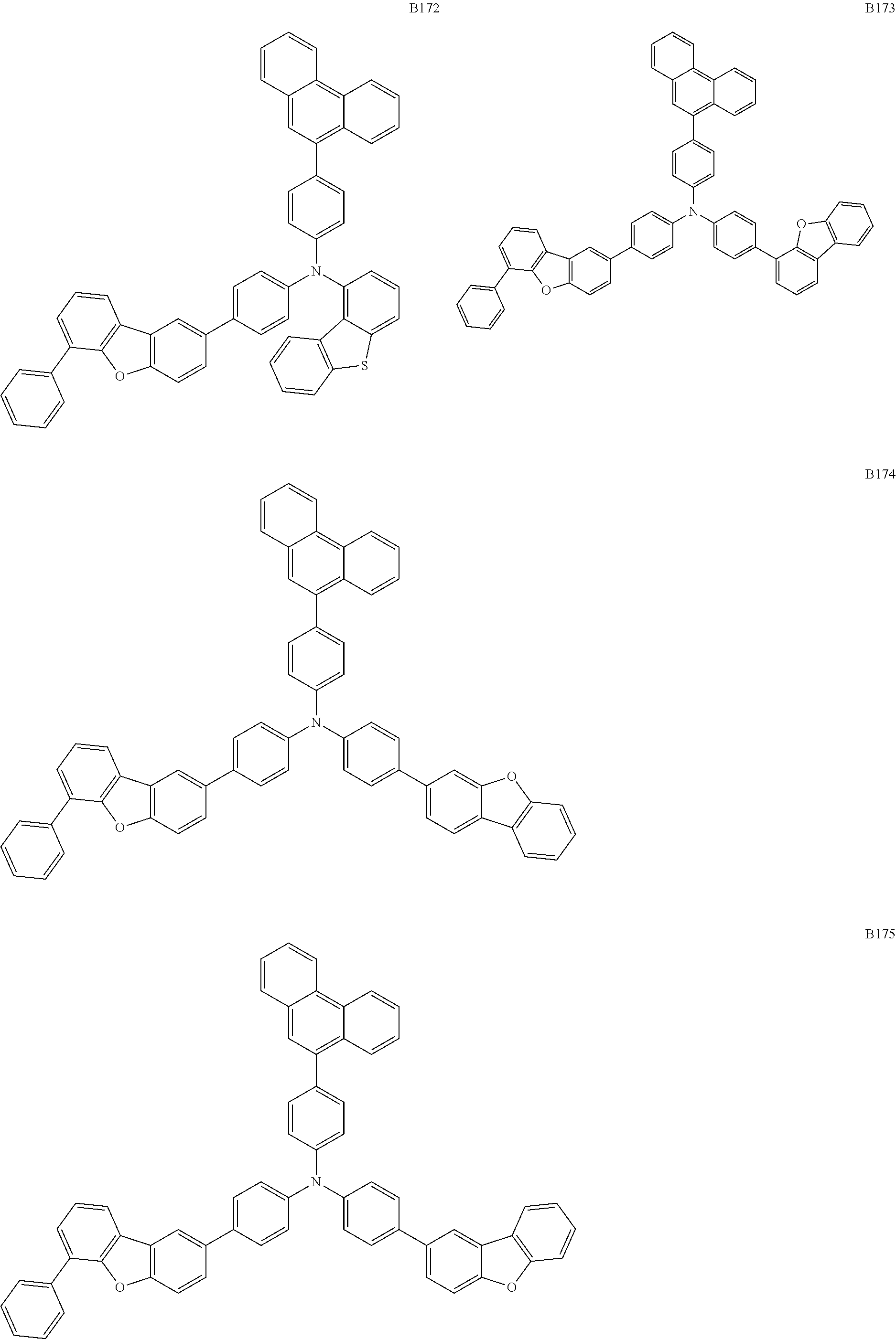 [SMILES: c1ccc(-c2cccc3c2oc2ccc(-c4ccc(N(c5ccc(-c6cc7ccccc7c7ccccc67)cc5)c5cccc6sc7ccccc7c56)cc4)cc23)cc1 ; c1ccc(-c2cccc3c2oc2ccc(-c4ccc(N(c5ccc(-c6cc7ccccc7c7ccccc67)cc5)c5ccc(-c6cccc7c6oc6ccccc67)cc5)cc4)cc23)cc1 ; c1ccc(-c2cccc3c2oc2ccc(-c4ccc(N(c5ccc(-c6ccc7c(c6)oc6ccccc67)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7oc8ccccc8c7c6)cc5)c5ccc(-c6cc7ccccc7c7ccccc67)cc5)cc4)cc23)cc1]

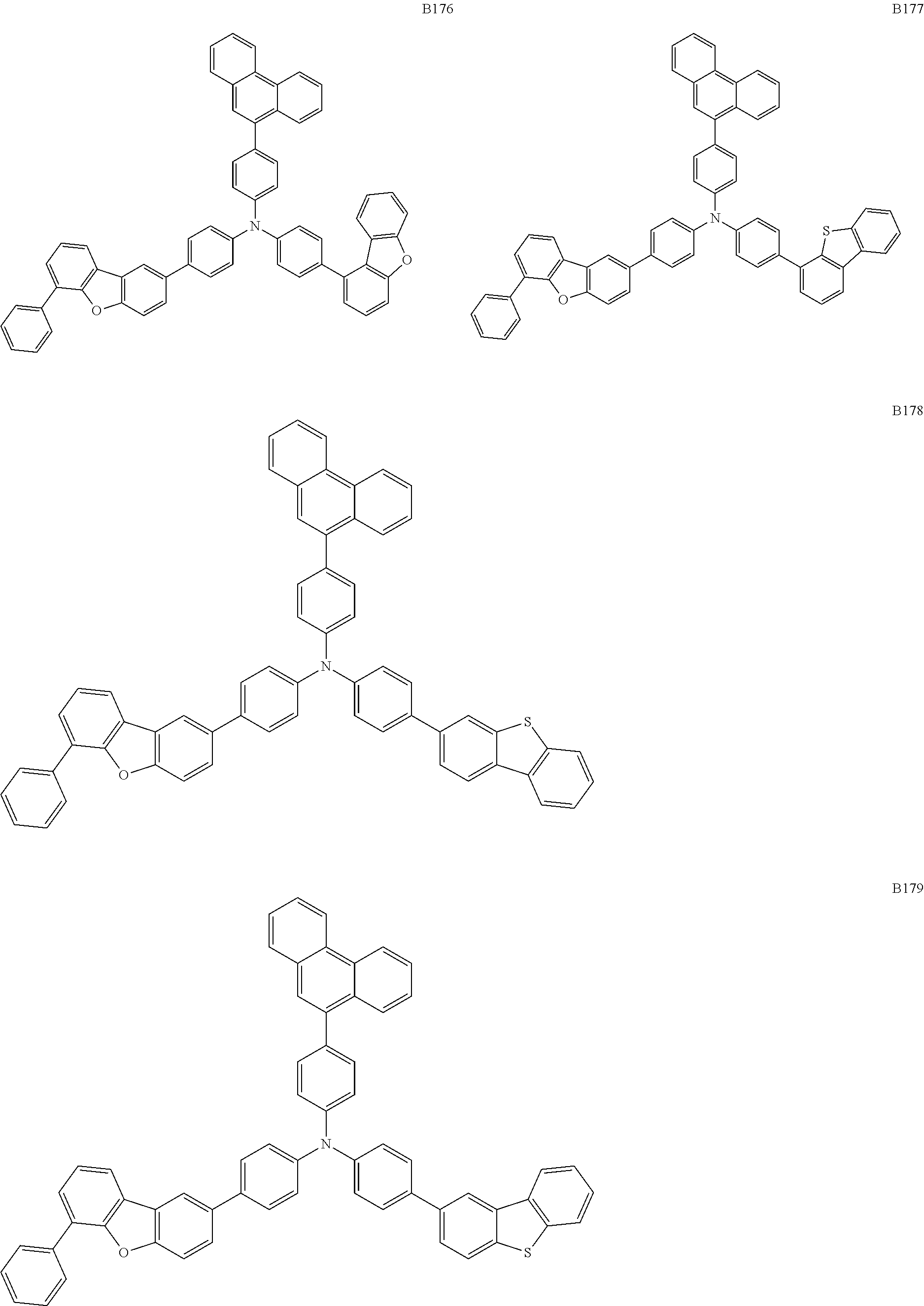 [SMILES: c1ccc(-c2cccc3c2oc2ccc(-c4ccc(N(c5ccc(-c6cc7ccccc7c7ccccc67)cc5)c5ccc(-c6cccc7oc8ccccc8c67)cc5)cc4)cc23)cc1 ; c1ccc(-c2cccc3c2oc2ccc(-c4ccc(N(c5ccc(-c6cc7ccccc7c7ccccc67)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7sc8ccccc8c7c6)cc5)c5ccc(-c6cc7ccccc7c7ccccc67)cc5)cc4)cc23)cc1]

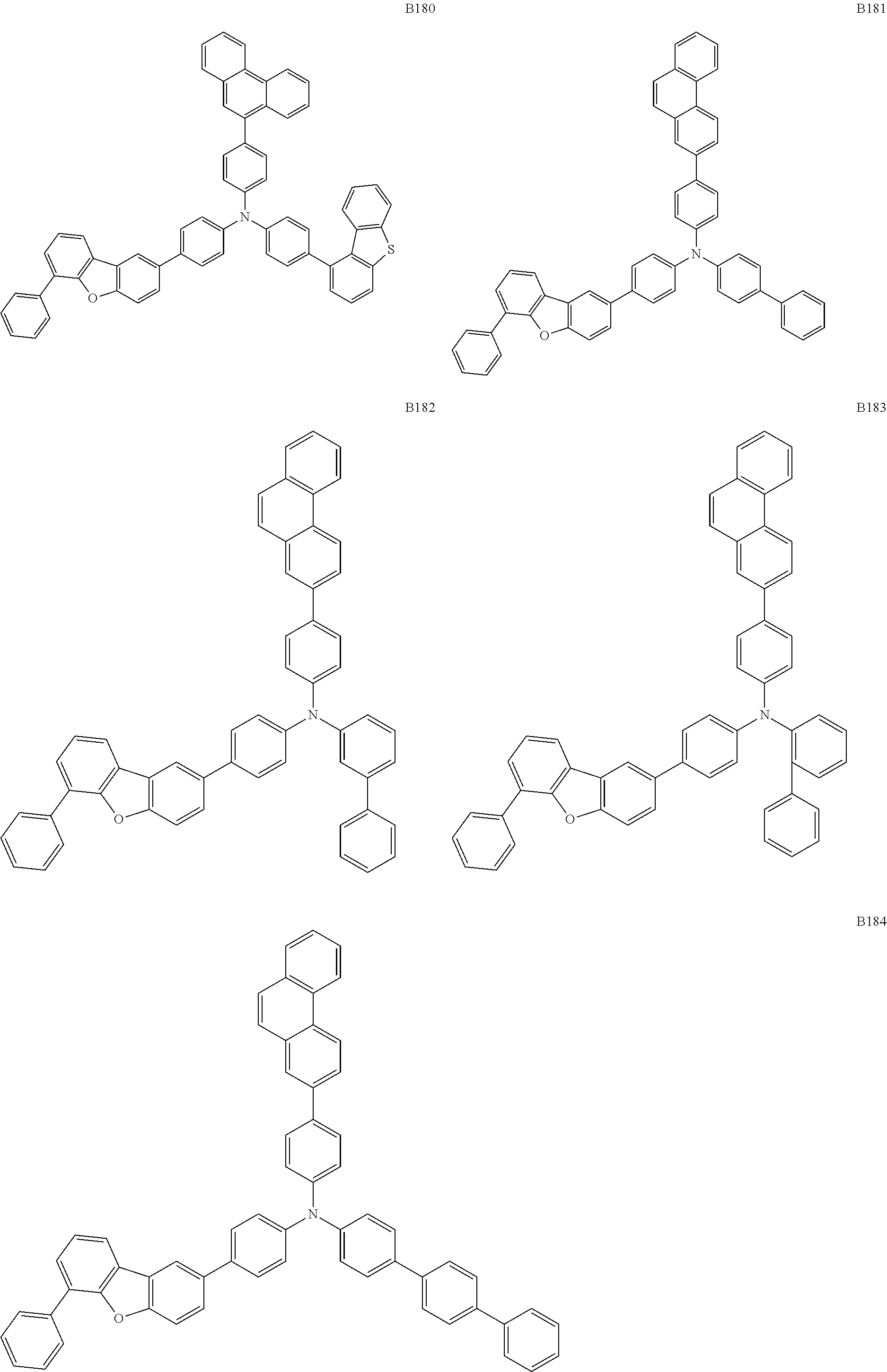

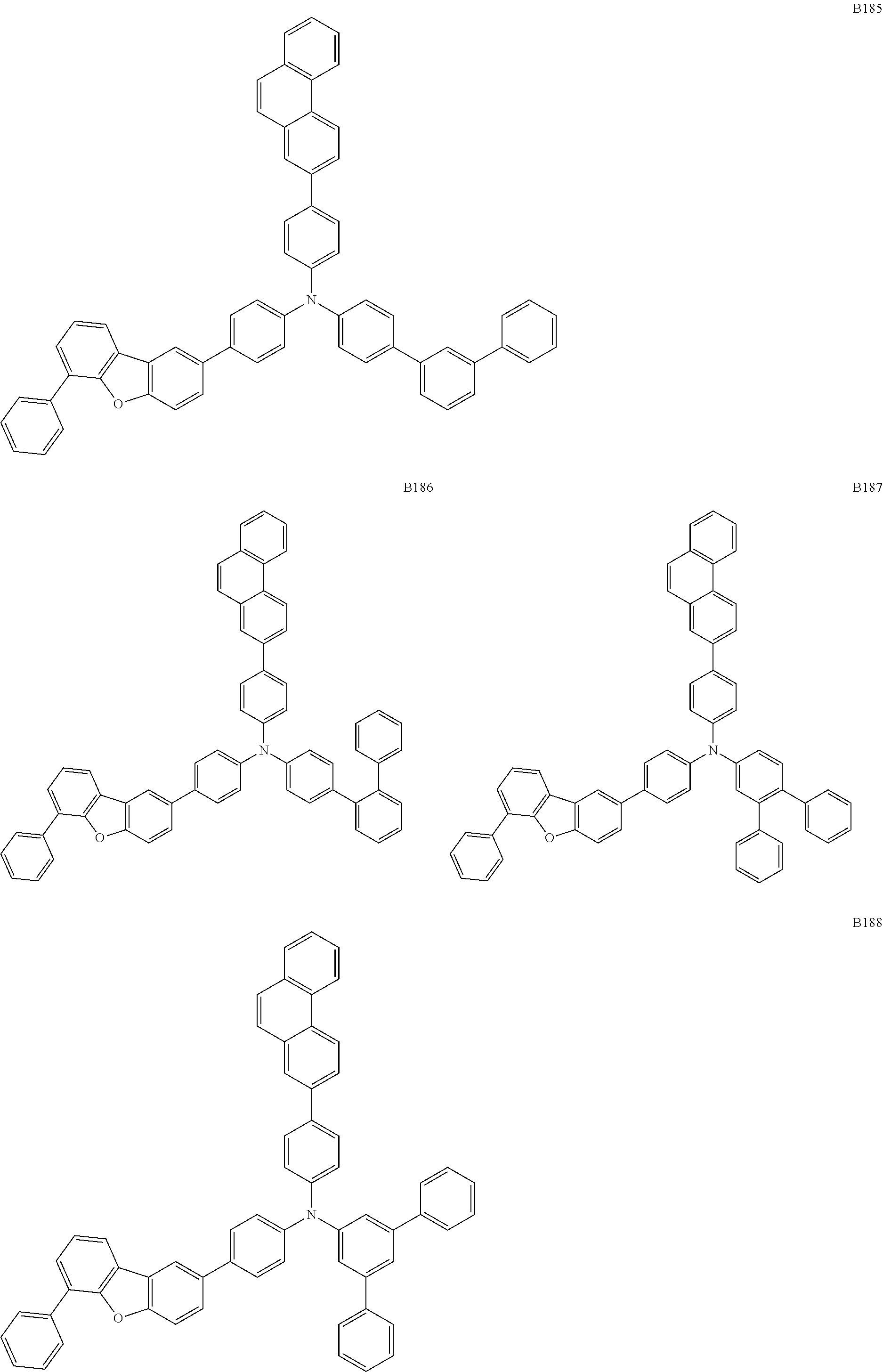 [SMILES: c1ccc(-c2cccc(-c3ccc(N(c4ccc(-c5ccc6c(ccc7ccccc76)c5)cc4)c4ccc(-c5ccc6oc7c(-c8ccccc8)cccc7c6c5)cc4)cc3)c2)cc1 ; c1ccc(-c2ccccc2-c2ccc(N(c3ccc(-c4ccc5c(ccc6ccccc65)c4)cc3)c3ccc(-c4ccc5oc6c(-c7ccccc7)cccc6c5c4)cc3)cc2)cc1 ; c1ccc(-c2ccc(N(c3ccc(-c4ccc5c(ccc6ccccc65)c4)cc3)c3ccc(-c4ccc5oc6c(-c7ccccc7)cccc6c5c4)cc3)cc2-c2ccccc2)cc1 ; c1ccc(-c2cc(-c3ccccc3)cc(N(c3ccc(-c4ccc5c(ccc6ccccc65)c4)cc3)c3ccc(-c4ccc5oc6c(-c7ccccc7)cccc6c5c4)cc3)c2)cc1]

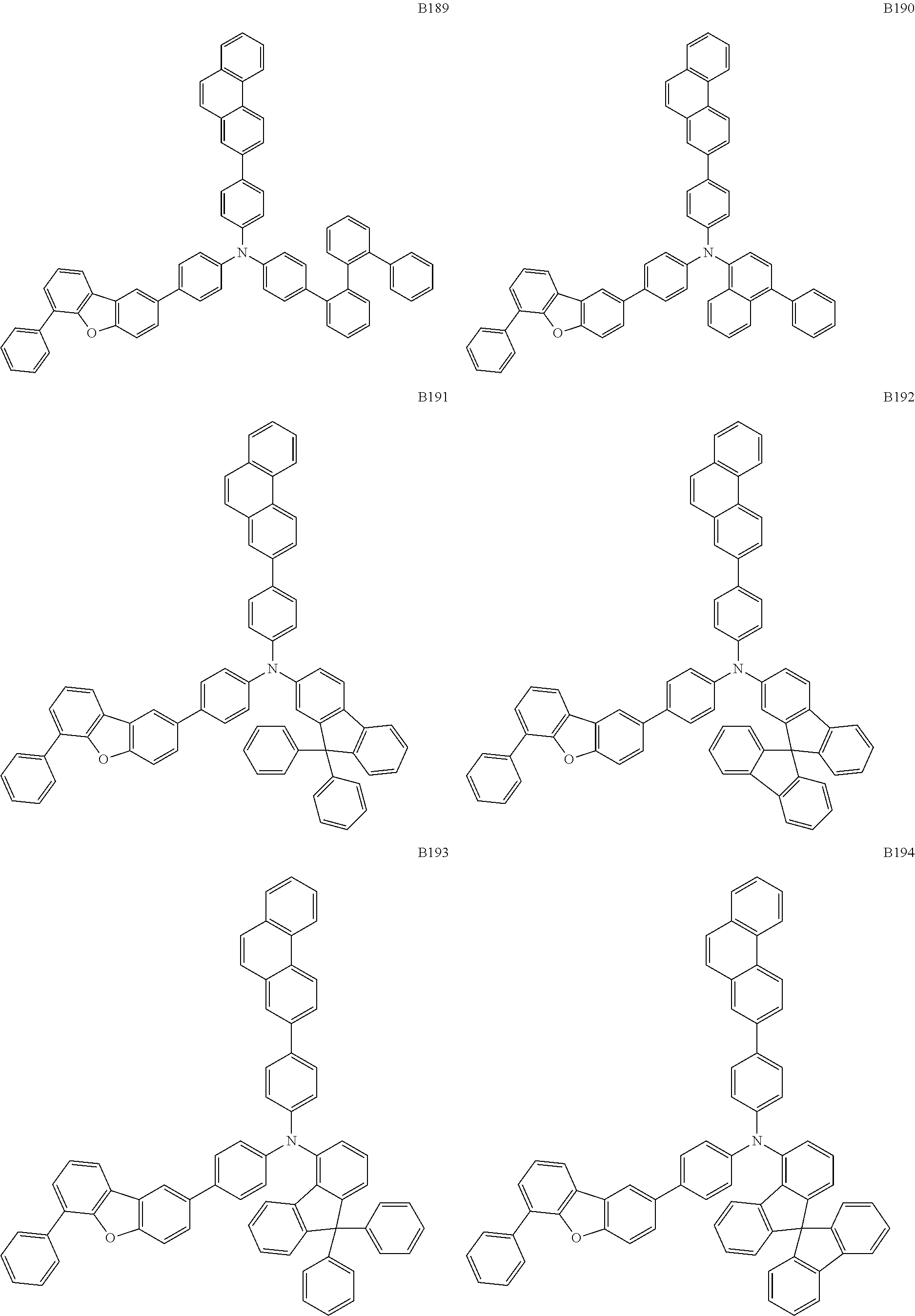

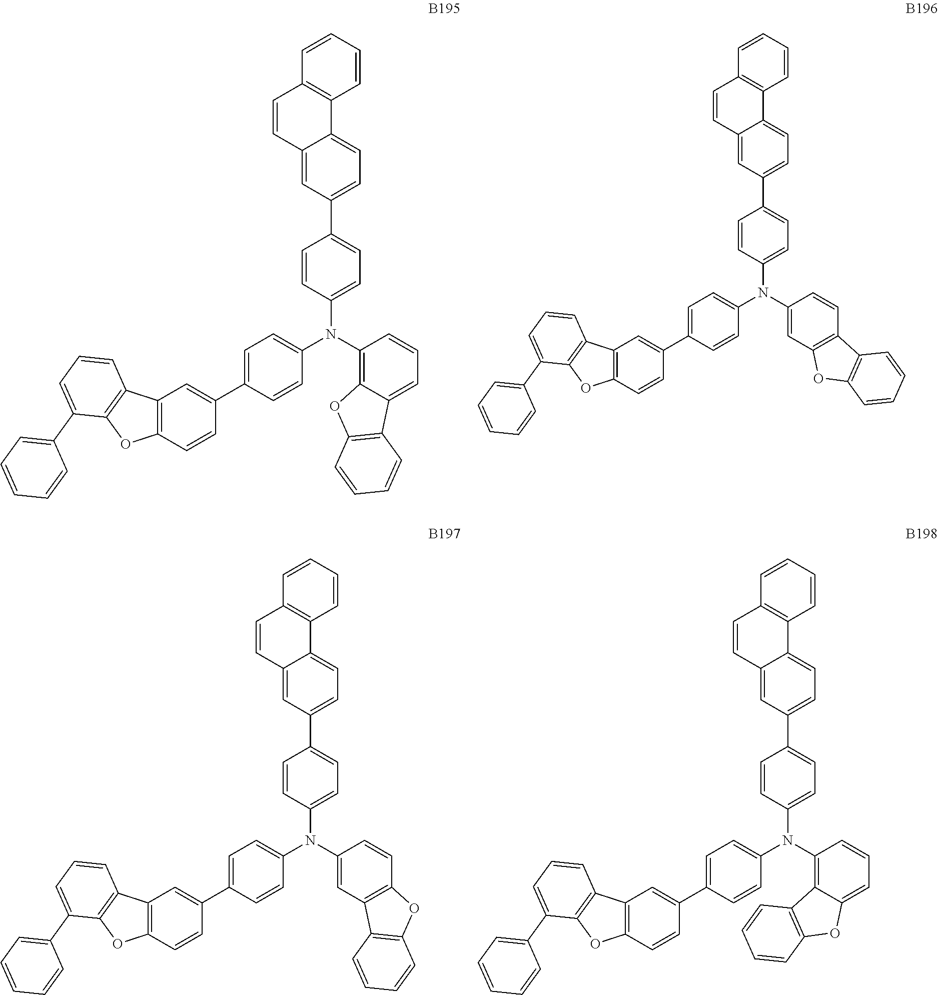 [SMILES: c1ccc(-c2cccc3c2oc2ccc(-c4ccc(N(c5ccc(-c6ccc7c(ccc8ccccc87)c6)cc5)c5cccc6c5oc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6c(c5)oc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6oc7ccccc7c6c5)cc4)cc23)cc1 ; c1ccc(-c2cccc3c2oc2ccc(-c4ccc(N(c5ccc(-c6ccc7c(ccc8ccccc87)c6)cc5)c5cccc6oc7ccccc7c56)cc4)cc23)cc1]

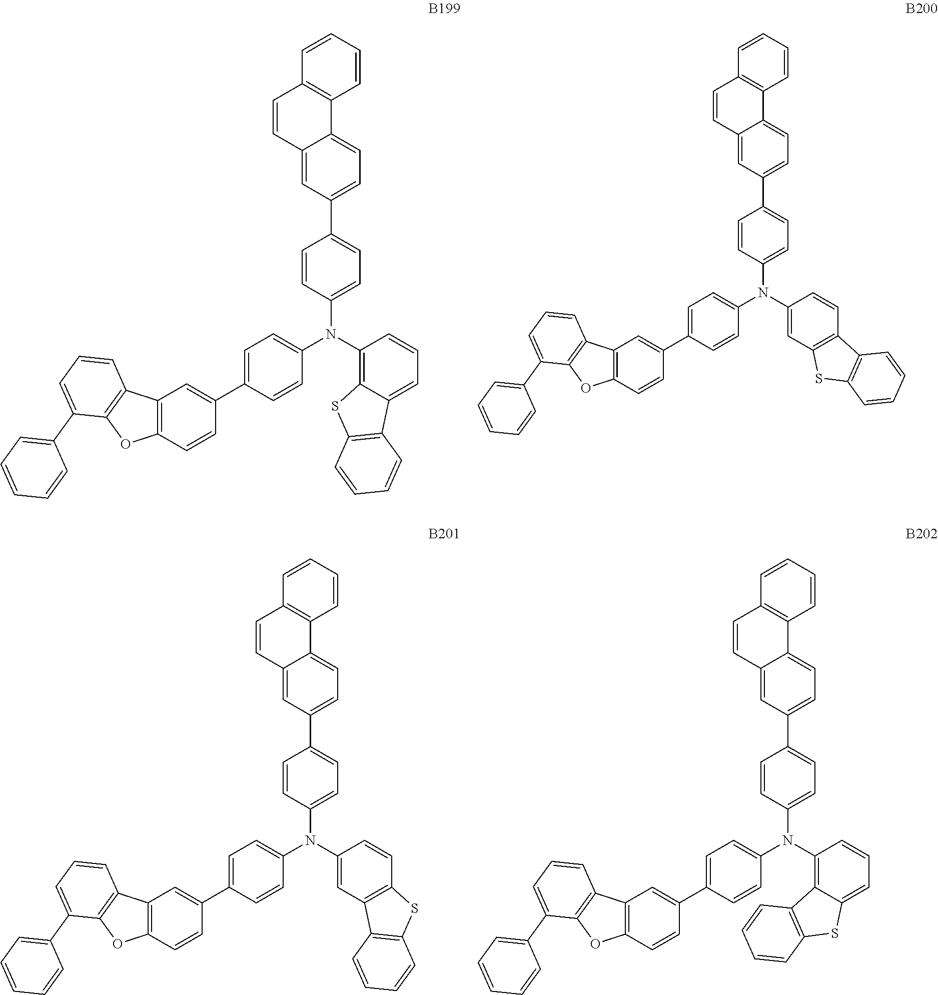 [SMILES: c1ccc(-c2cccc3c2oc2ccc(-c4ccc(N(c5ccc(-c6ccc7c(ccc8ccccc87)c6)cc5)c5cccc6c5sc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6c(c5)sc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6sc7ccccc7c6c5)cc4)cc23)cc1 ; c1ccc(-c2cccc3c2oc2ccc(-c4ccc(N(c5ccc(-c6ccc7c(ccc8ccccc87)c6)cc5)c5cccc6sc7ccccc7c56)cc4)cc23)cc1]

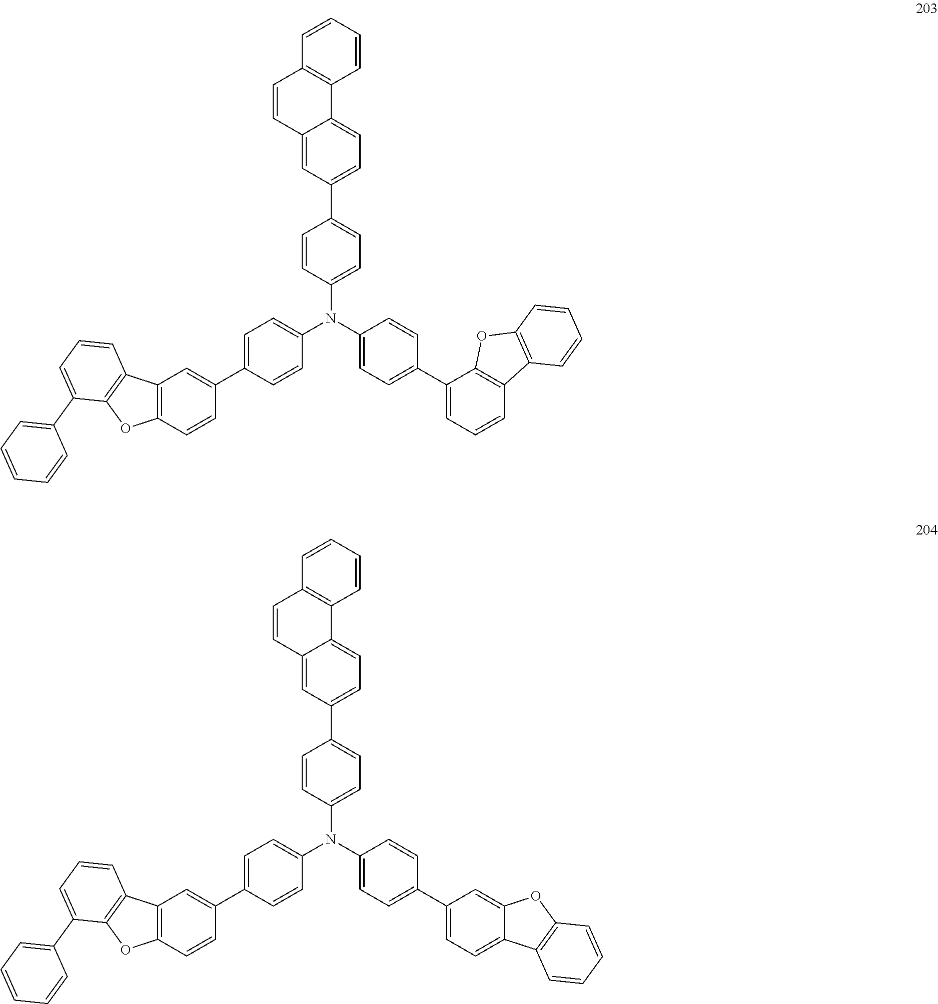 [SMILES: c1ccc(-c2cccc3c2oc2ccc(-c4ccc(N(c5ccc(-c6ccc7c(ccc8ccccc87)c6)cc5)c5ccc(-c6cccc7c6oc6ccccc67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6ccc7c(c6)oc6ccccc67)cc5)cc4)cc23)cc1]

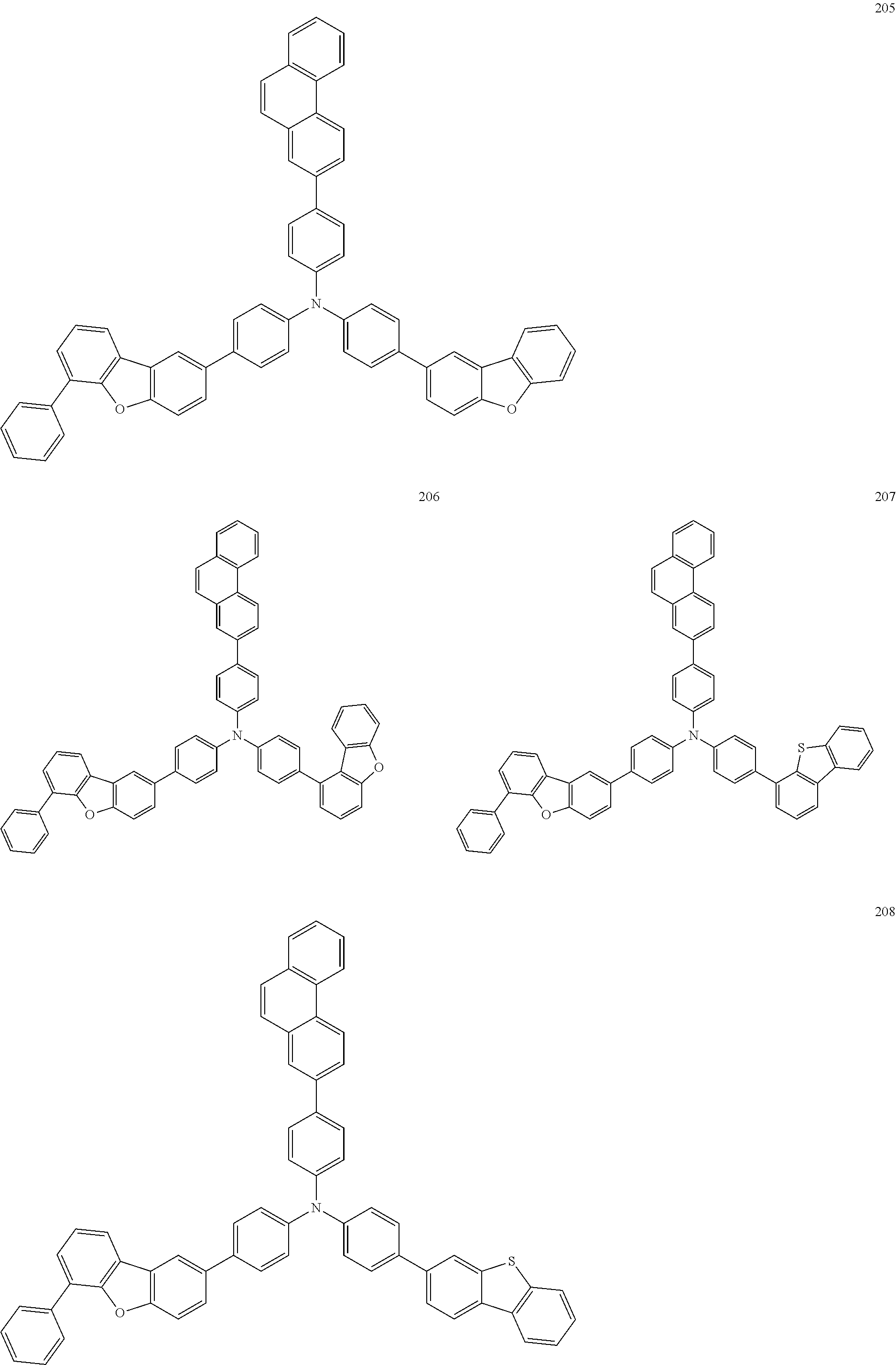 [SMILES: c1ccc(-c2cccc3c2oc2ccc(-c4ccc(N(c5ccc(-c6ccc7c(ccc8ccccc87)c6)cc5)c5ccc(-c6ccc7oc8ccccc8c7c6)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6ccc7c(c6)sc6ccccc67)cc5)cc4)cc23)cc1]

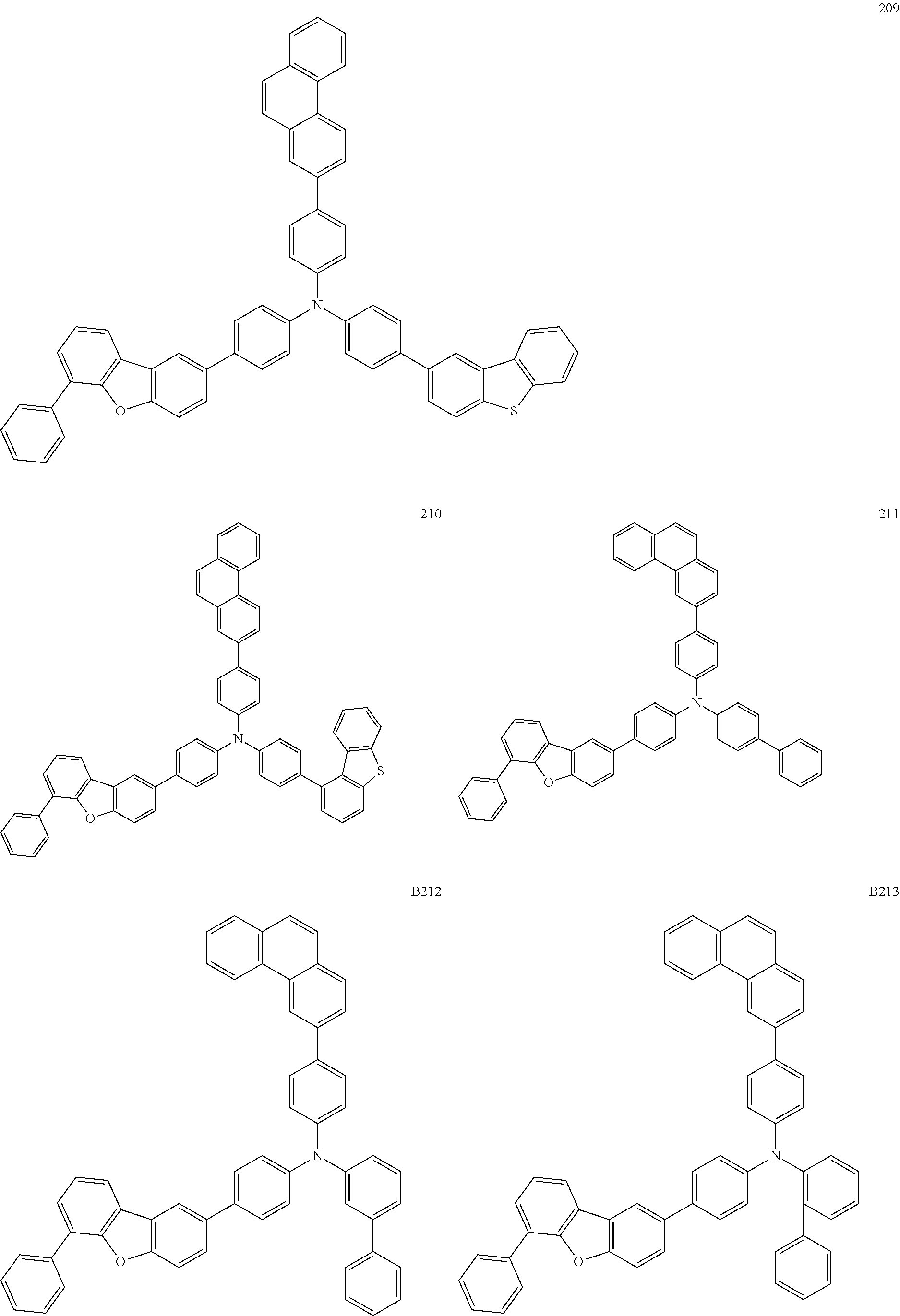

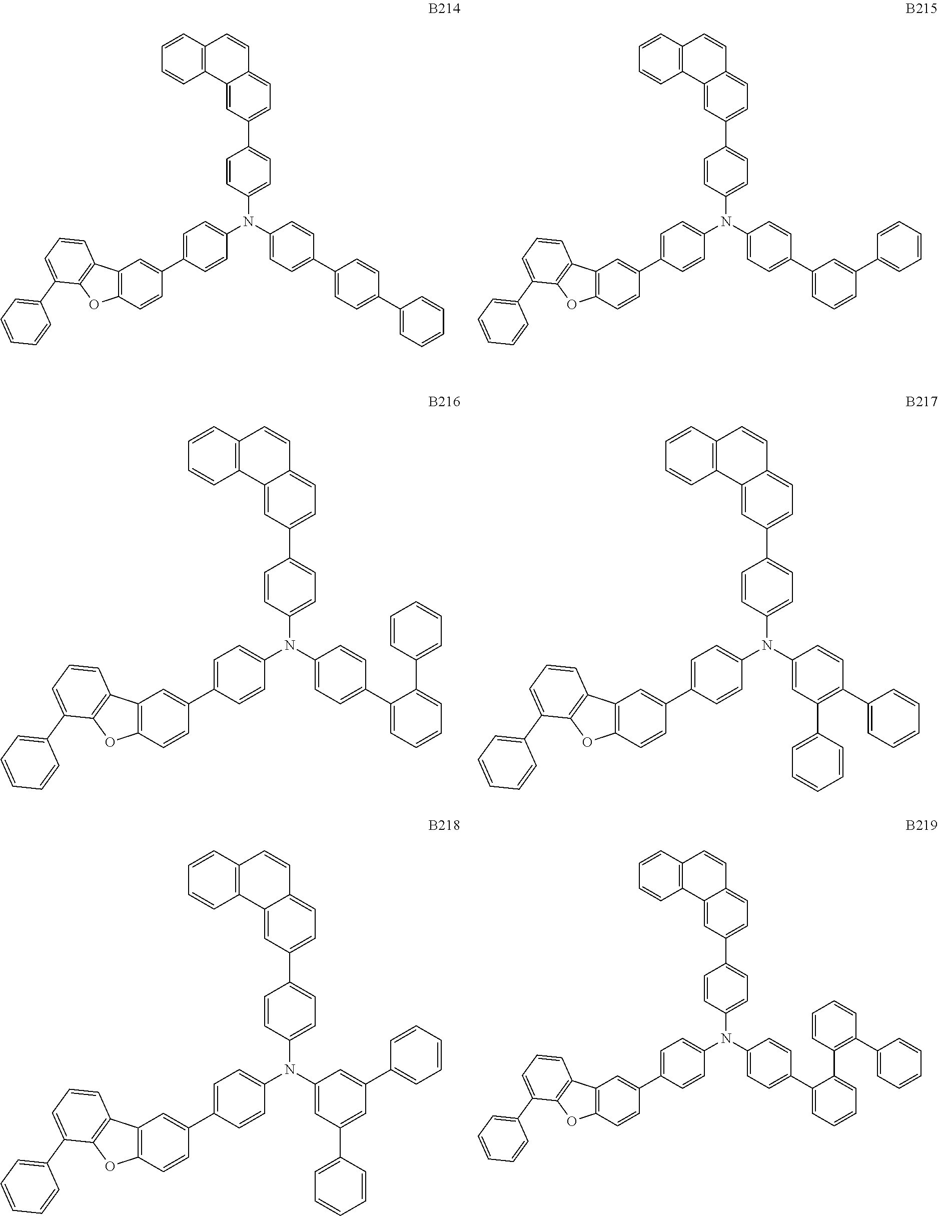

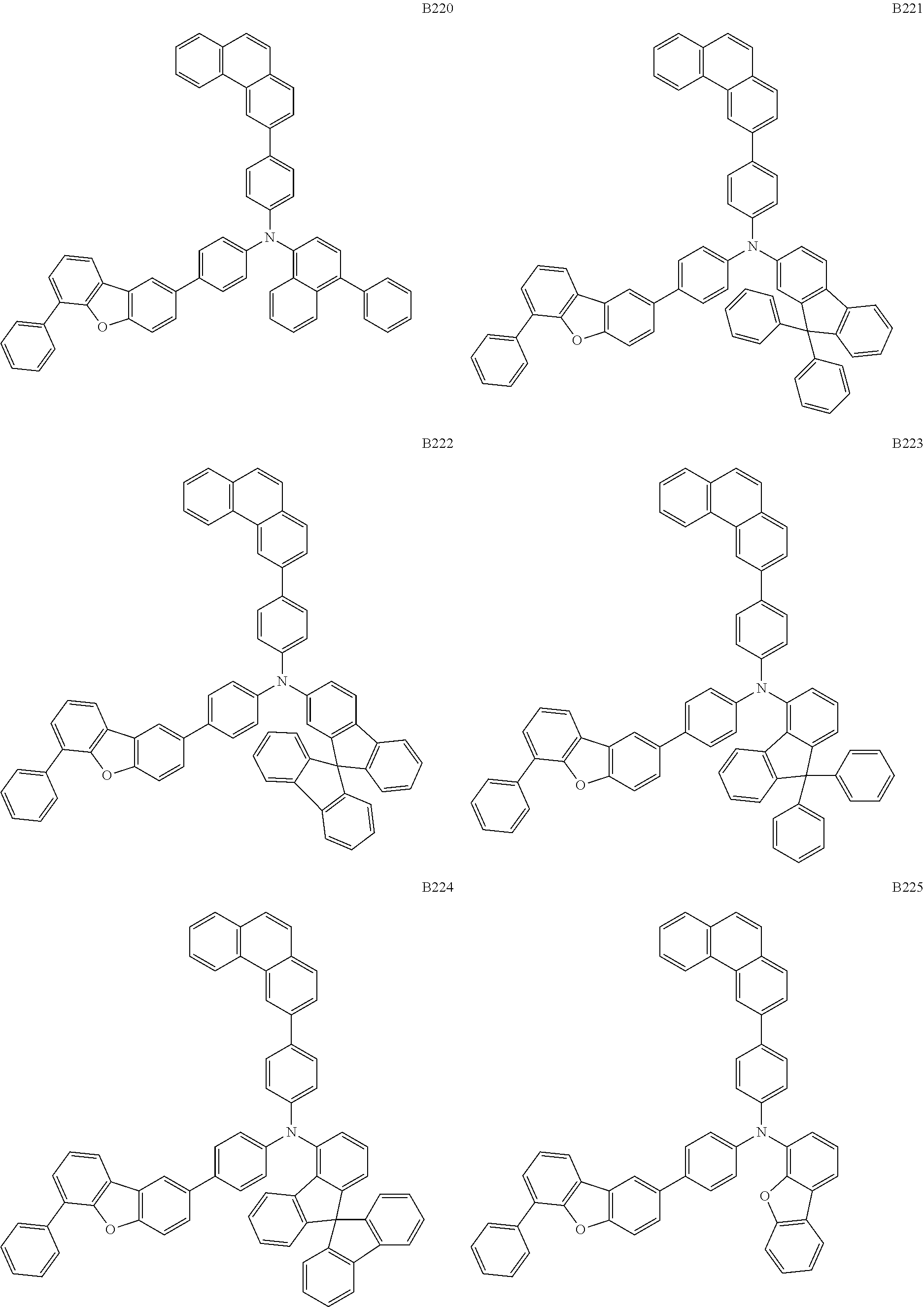

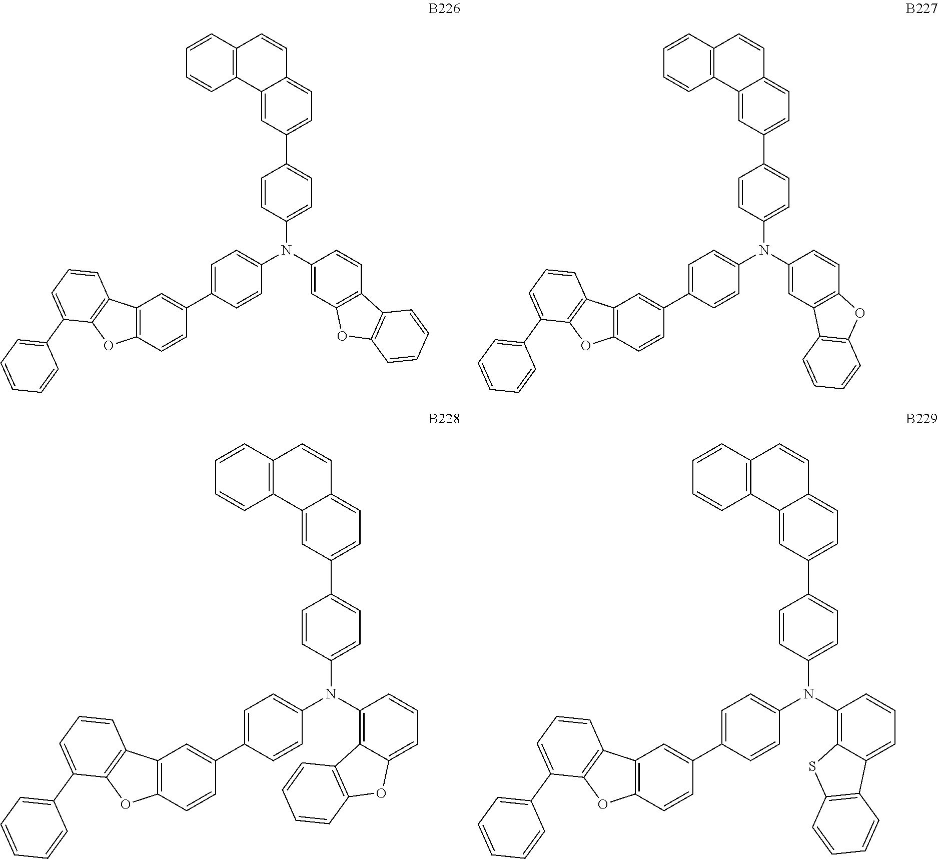 [SMILES: c1ccc(-c2cccc3c2oc2ccc(-c4ccc(N(c5ccc(-c6ccc7ccc8ccccc8c7c6)cc5)c5ccc6c(c5)oc5ccccc56)cc4)cc23)cc1 ; c1ccc(-c2cccc3c2oc2ccc(-c4ccc(N(c5ccc(-c6ccc7ccc8ccccc8c7c6)cc5)c5ccc6oc7ccccc7c6c5)cc4)cc23)cc1 ; c1ccc(-c2cccc3c2oc2ccc(-c4ccc(N(c5ccc(-c6ccc7ccc8ccccc8c7c6)cc5)c5cccc6oc7ccccc7c56)cc4)cc23)cc1 ; c1ccc(-c2cccc3c2oc2ccc(-c4ccc(N(c5ccc(-c6ccc7ccc8ccccc8c7c6)cc5)c5cccc6c5sc5ccccc56)cc4)cc23)cc1]

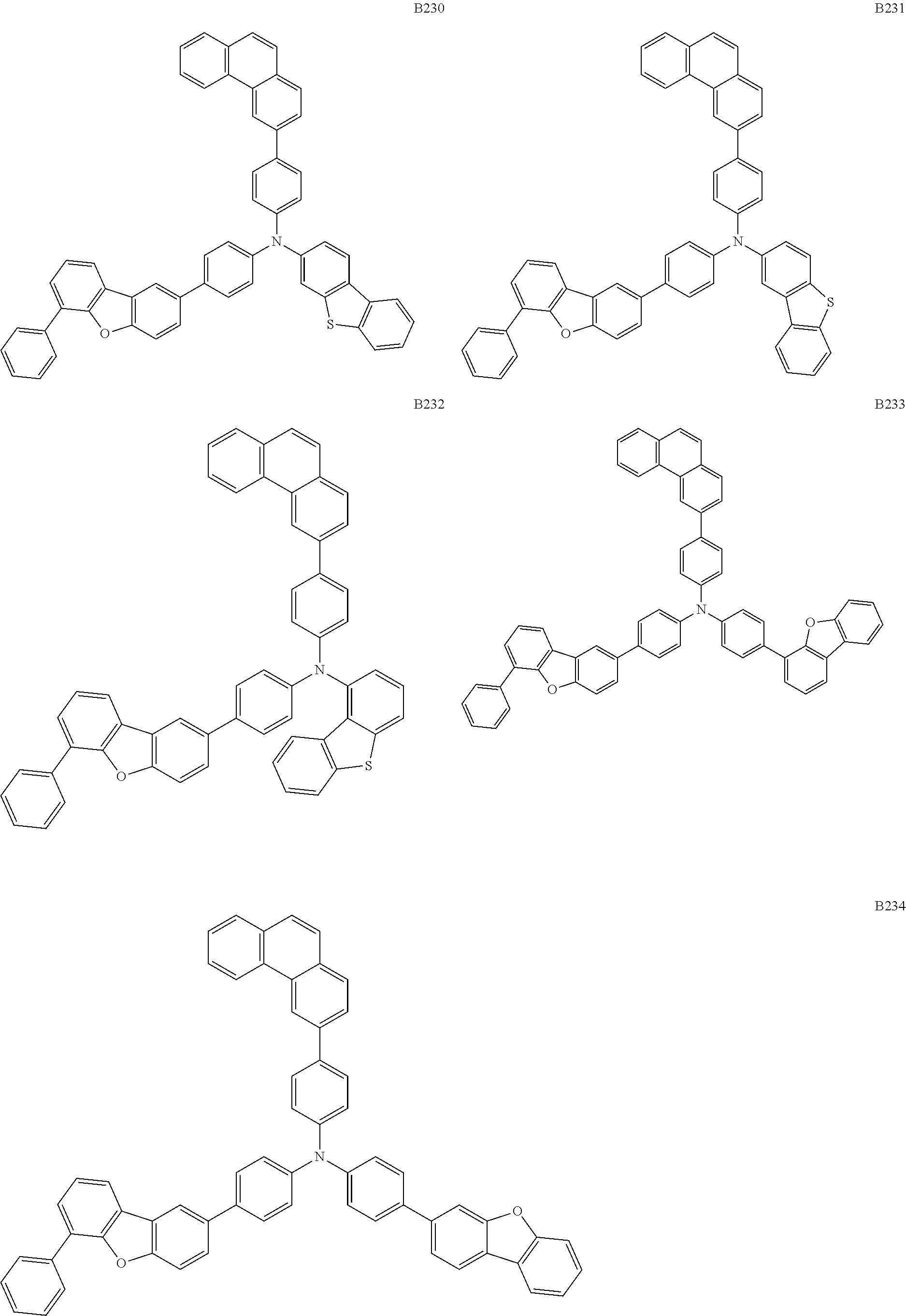

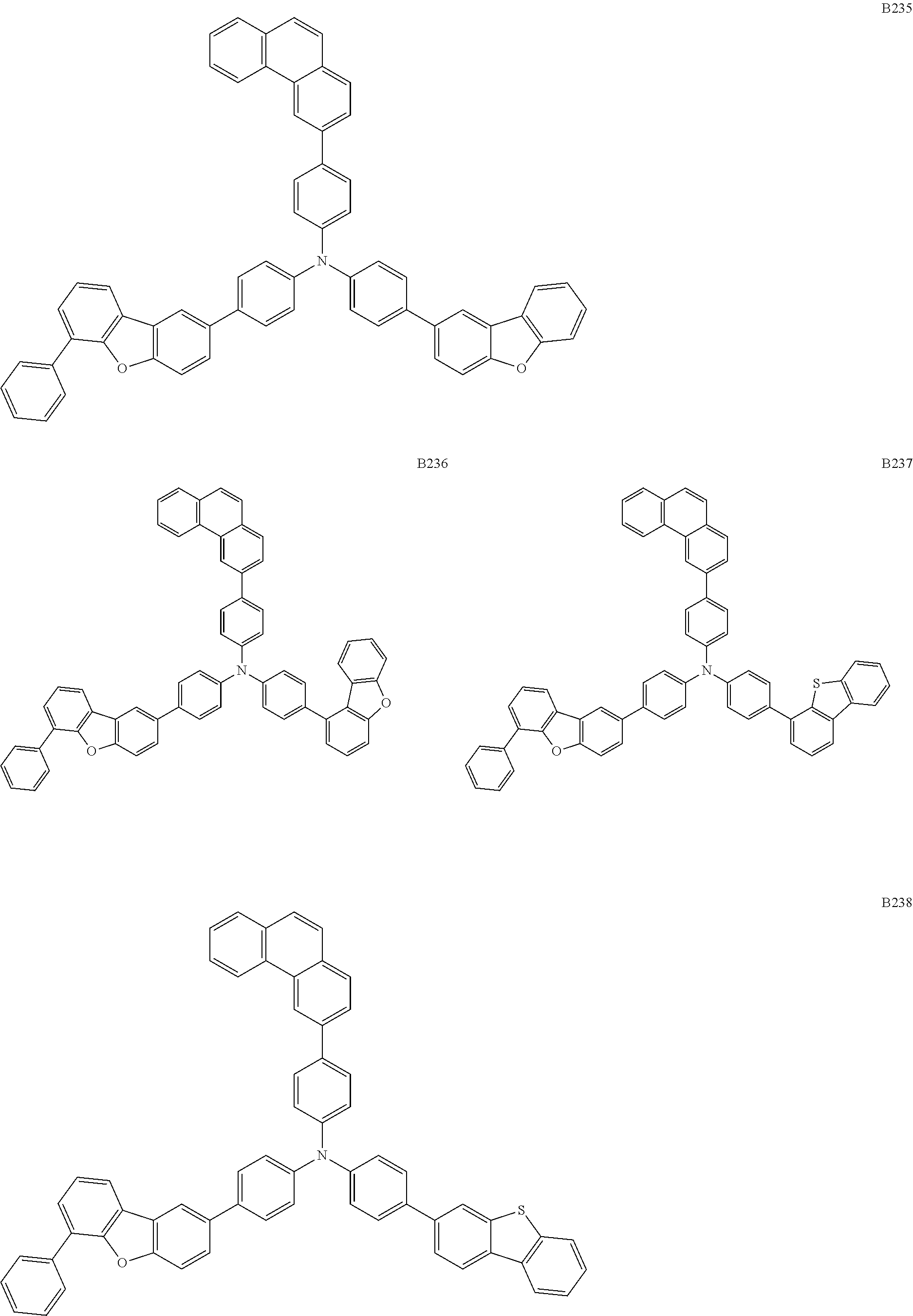 [SMILES: c1ccc(-c2cccc3c2oc2ccc(-c4ccc(N(c5ccc(-c6ccc7ccc8ccccc8c7c6)cc5)c5ccc(-c6ccc7oc8ccccc8c7c6)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6ccc7ccc8ccccc8c7c6)cc5)cc4)cc23)cc1]

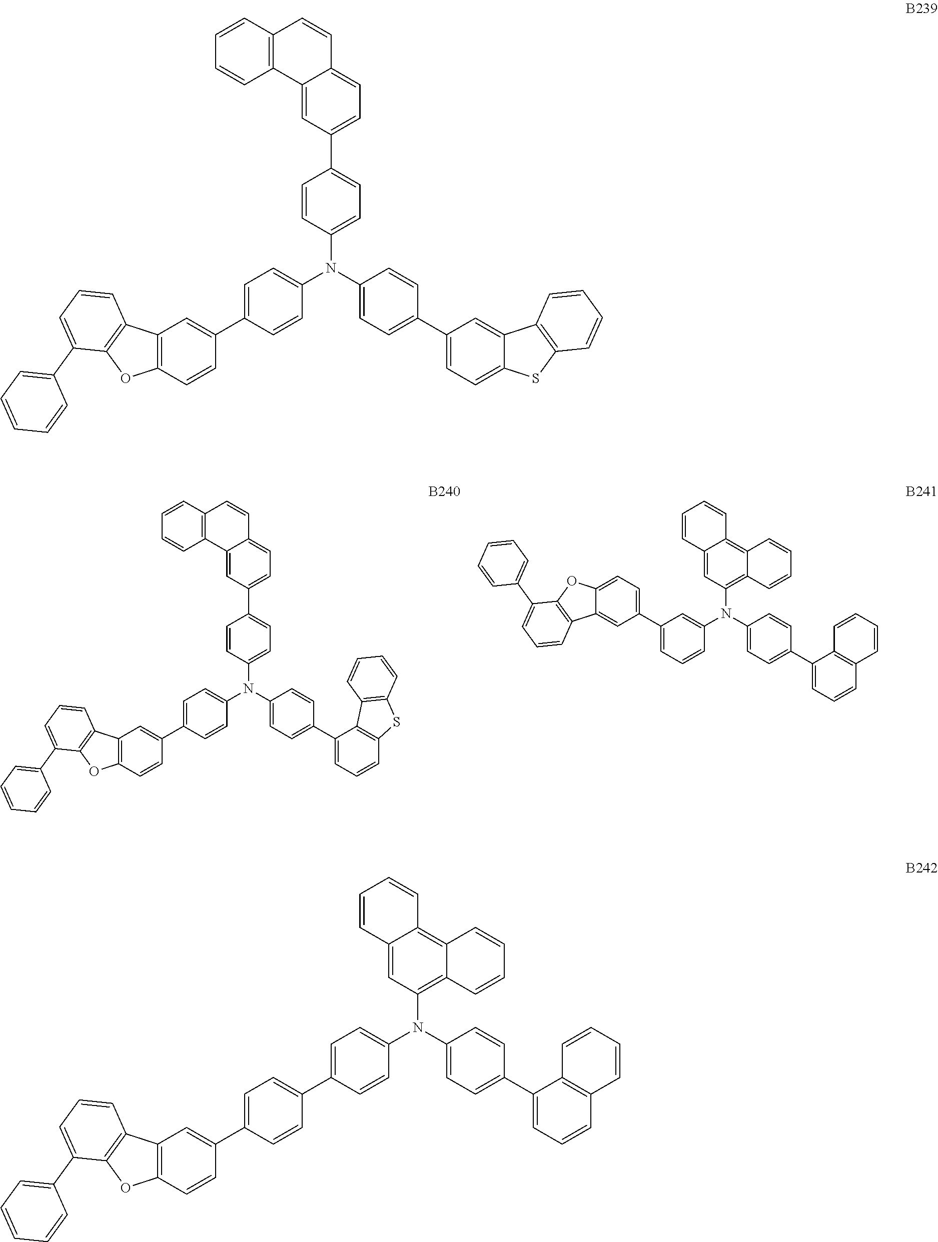 [SMILES: c1ccc(-c2cccc3c2oc2ccc(-c4ccc(N(c5ccc(-c6ccc7ccc8ccccc8c7c6)cc5)c5ccc(-c6ccc7sc8ccccc8c7c6)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7sc8ccccc8c67)cc5)cc4)cc23)cc1 ; c1ccc(-c2cccc3c2oc2ccc(-c4cccc(N(c5ccc(-c6cccc7ccccc67)cc5)c5cc6ccccc6c6ccccc56)c4)cc23)cc1 ; c1ccc(-c2cccc3c2oc2ccc(-c4ccc(-c5ccc(N(c6ccc(-c7cccc8ccccc78)cc6)c6cc7ccccc7c7ccccc67)cc5)cc4)cc23)cc1]

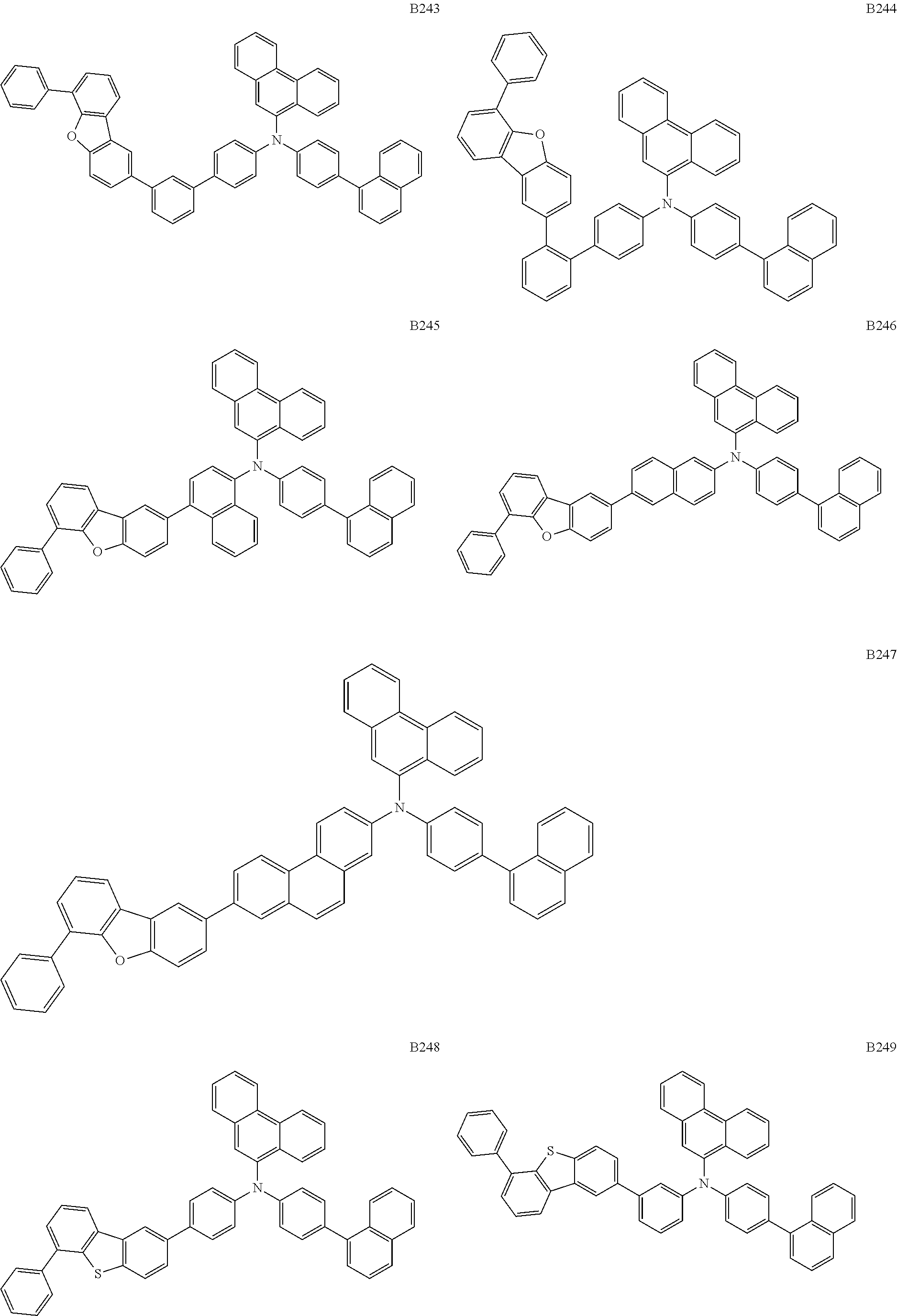

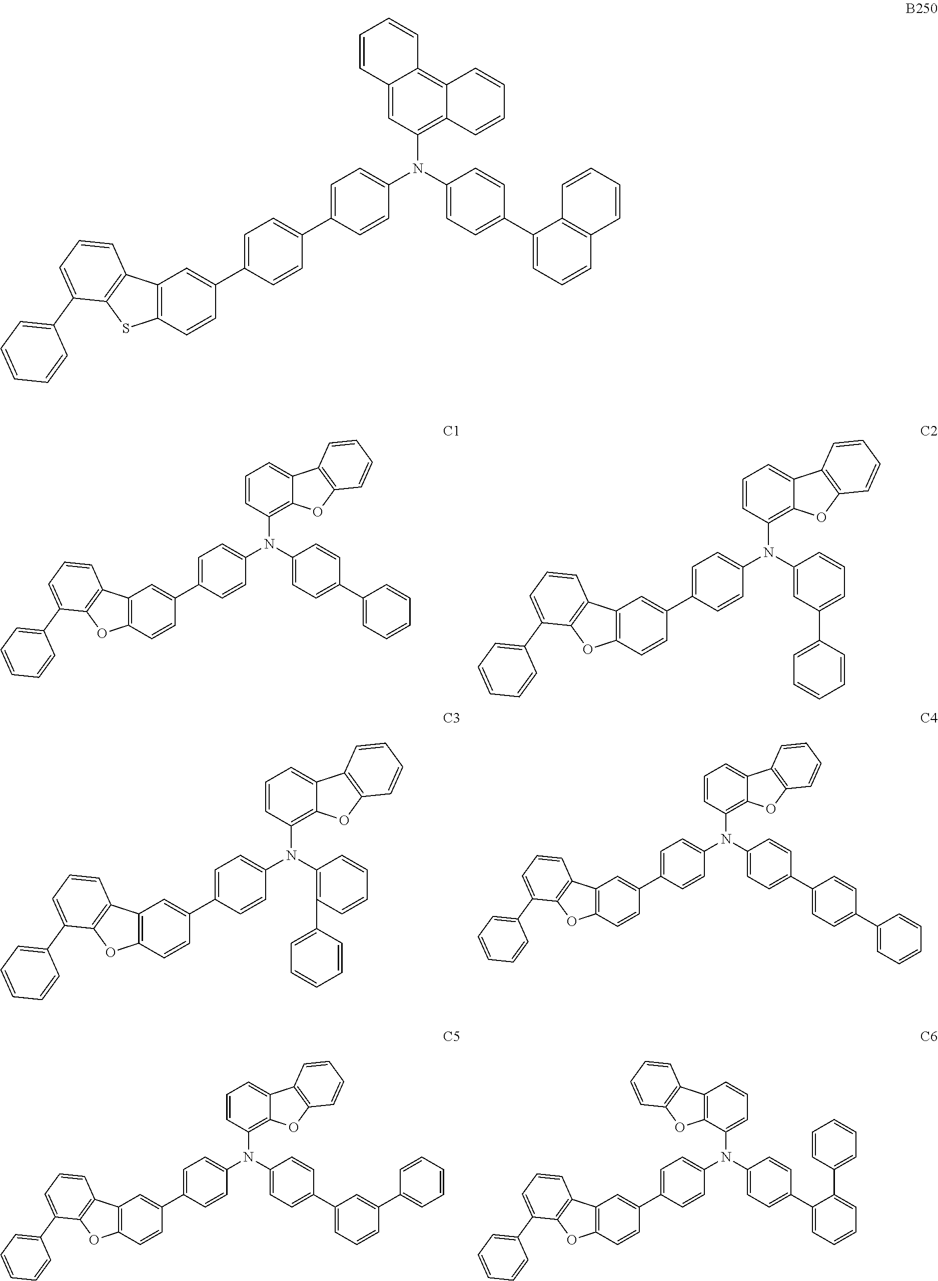

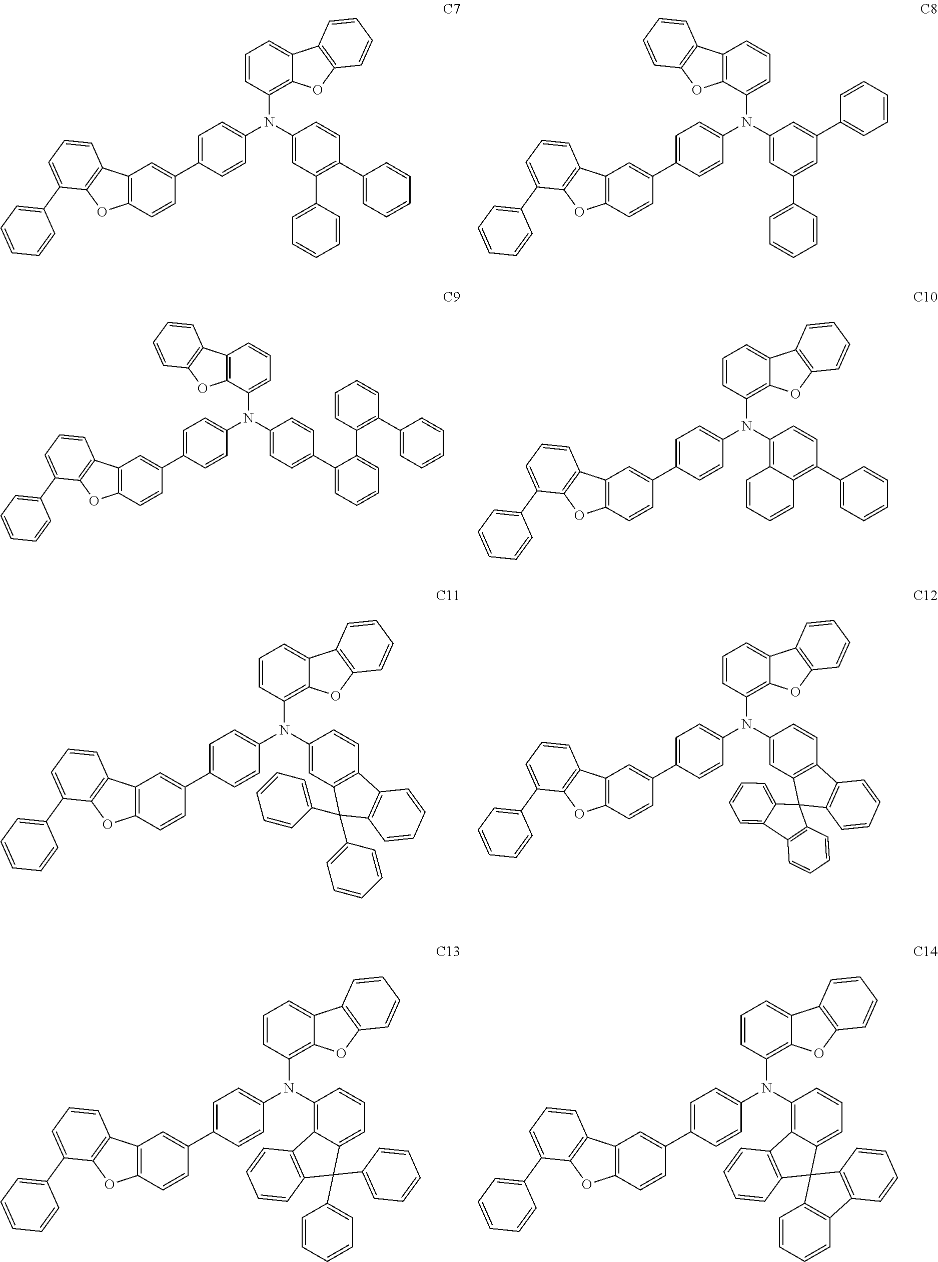

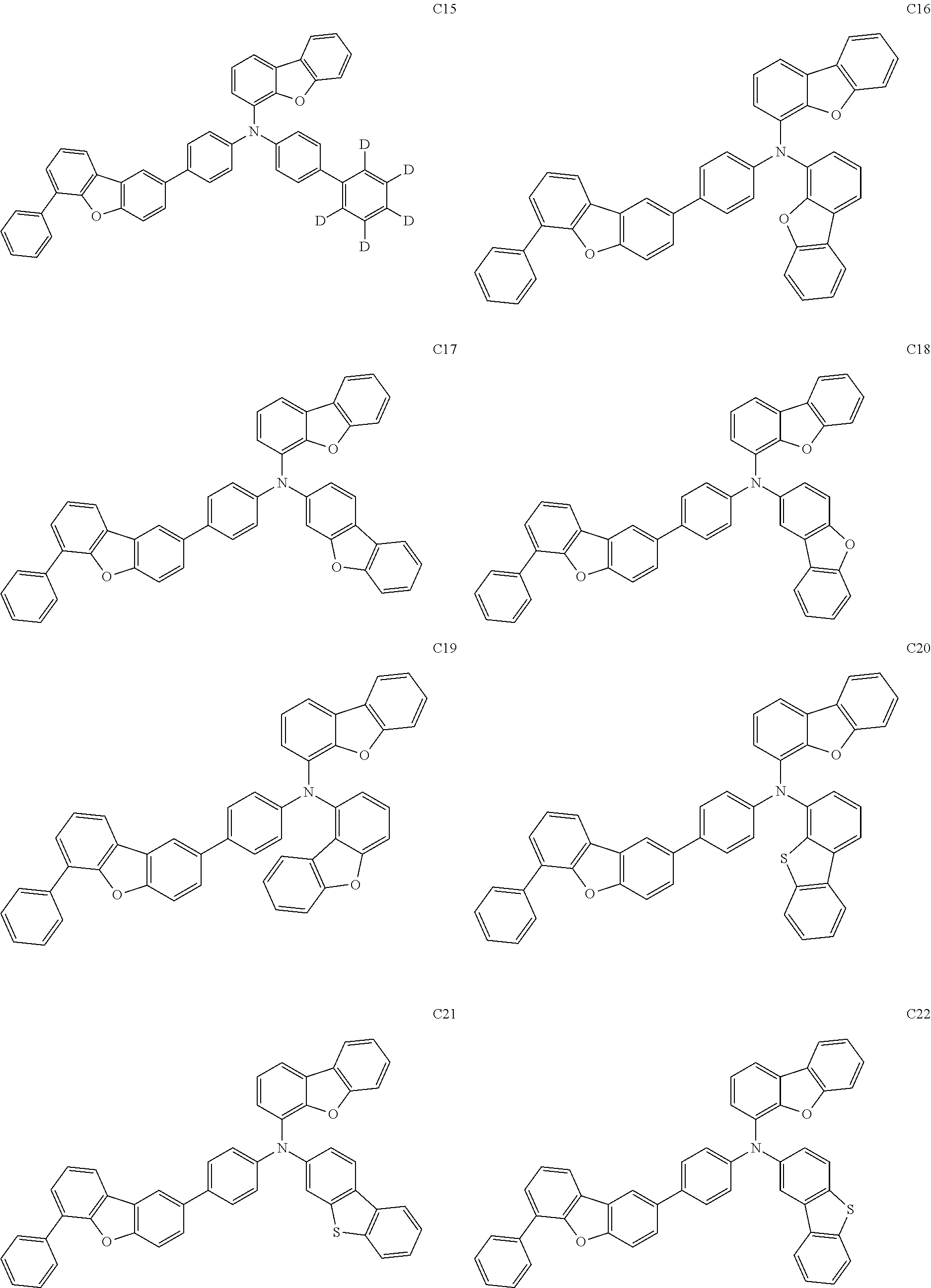

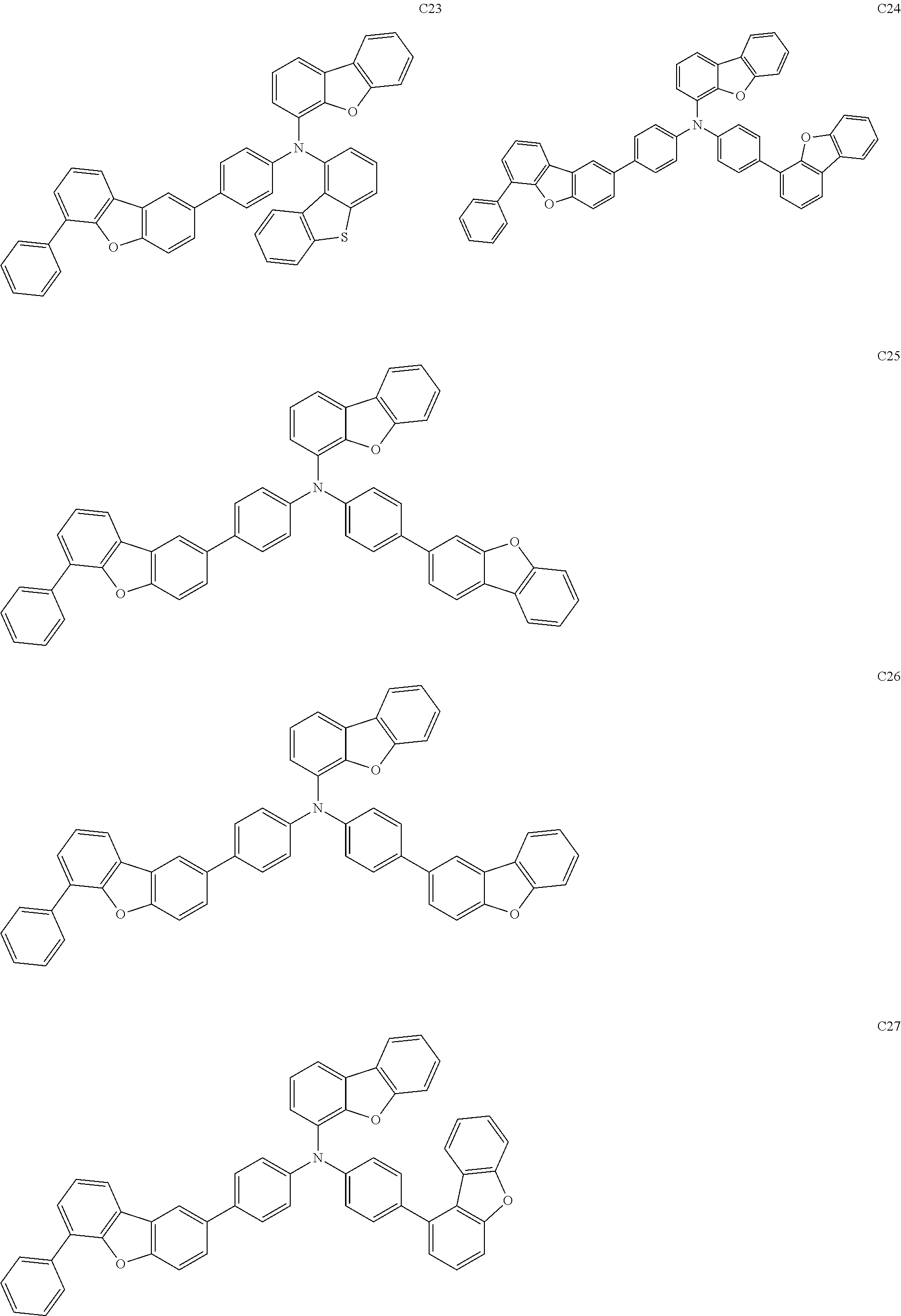 [SMILES: c1ccc(-c2cccc3c2oc2ccc(-c4ccc(N(c5cccc6c5oc5ccccc56)c5cccc6sc7ccccc7c56)cc4)cc23)cc1 ; c1ccc(-c2cccc3c2oc2ccc(-c4ccc(N(c5ccc(-c6cccc7c6oc6ccccc67)cc5)c5cccc6c5oc5ccccc56)cc4)cc23)cc1 ; c1ccc(-c2cccc3c2oc2ccc(-c4ccc(N(c5ccc(-c6ccc7c(c6)oc6ccccc67)cc5)c5cccc6c5oc5ccccc56)cc4)cc23)cc1 ; c1ccc(-c2cccc3c2oc2ccc(-c4ccc(N(c5ccc(-c6ccc7oc8ccccc8c7c6)cc5)c5cccc6c5oc5ccccc56)cc4)cc23)cc1 ; c1ccc(-c2cccc3c2oc2ccc(-c4ccc(N(c5ccc(-c6cccc7oc8ccccc8c67)cc5)c5cccc6c5oc5ccccc56)cc4)cc23)cc1]

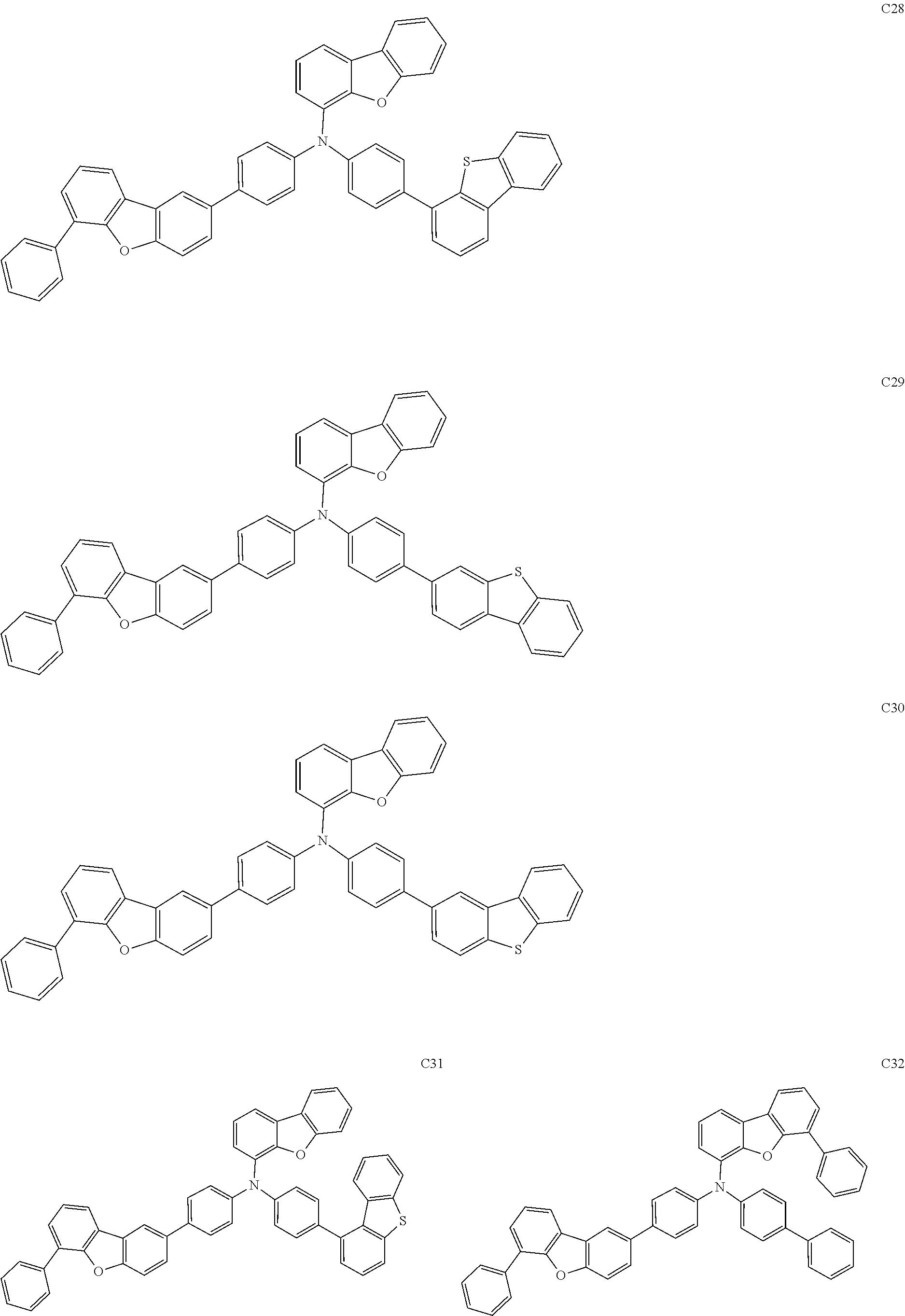 [SMILES: c1ccc(-c2cccc3c2oc2ccc(-c4ccc(N(c5ccc(-c6cccc7c6sc6ccccc67)cc5)c5cccc6c5oc5ccccc56)cc4)cc23)cc1 ; c1ccc(-c2cccc3c2oc2ccc(-c4ccc(N(c5ccc(-c6ccc7c(c6)sc6ccccc67)cc5)c5cccc6c5oc5ccccc56)cc4)cc23)cc1 ; c1ccc(-c2cccc3c2oc2ccc(-c4ccc(N(c5ccc(-c6ccc7sc8ccccc8c7c6)cc5)c5cccc6c5oc5ccccc56)cc4)cc23)cc1 ; c1ccc(-c2cccc3c2oc2ccc(-c4ccc(N(c5ccc(-c6cccc7sc8ccccc8c67)cc5)c5cccc6c5oc5ccccc56)cc4)cc23)cc1 ; c1ccc(-c2ccc(N(c3ccc(-c4ccc5oc6c(-c7ccccc7)cccc6c5c4)cc3)c3cccc4c3oc3c(-c5ccccc5)cccc34)cc2)cc1]

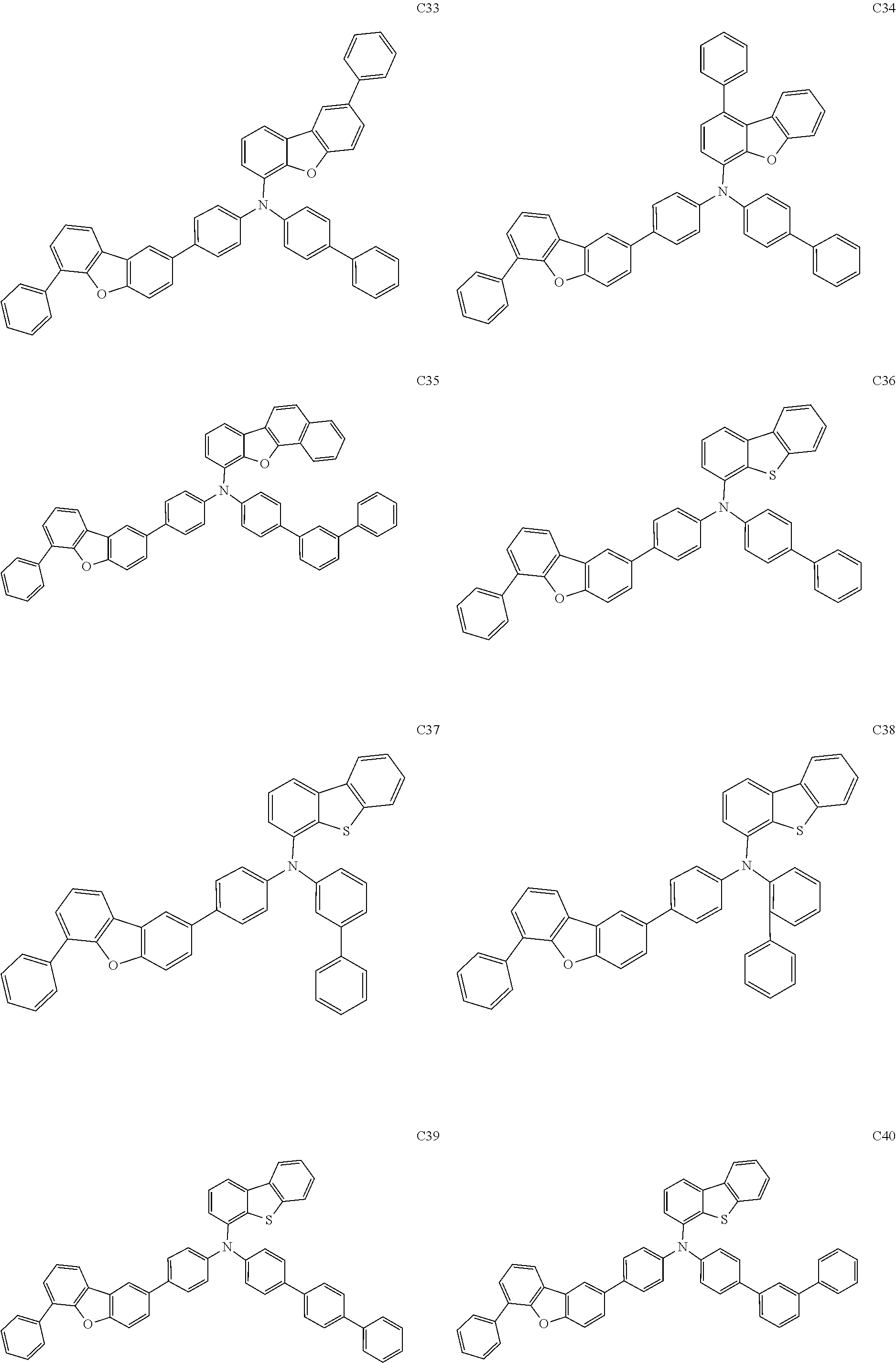

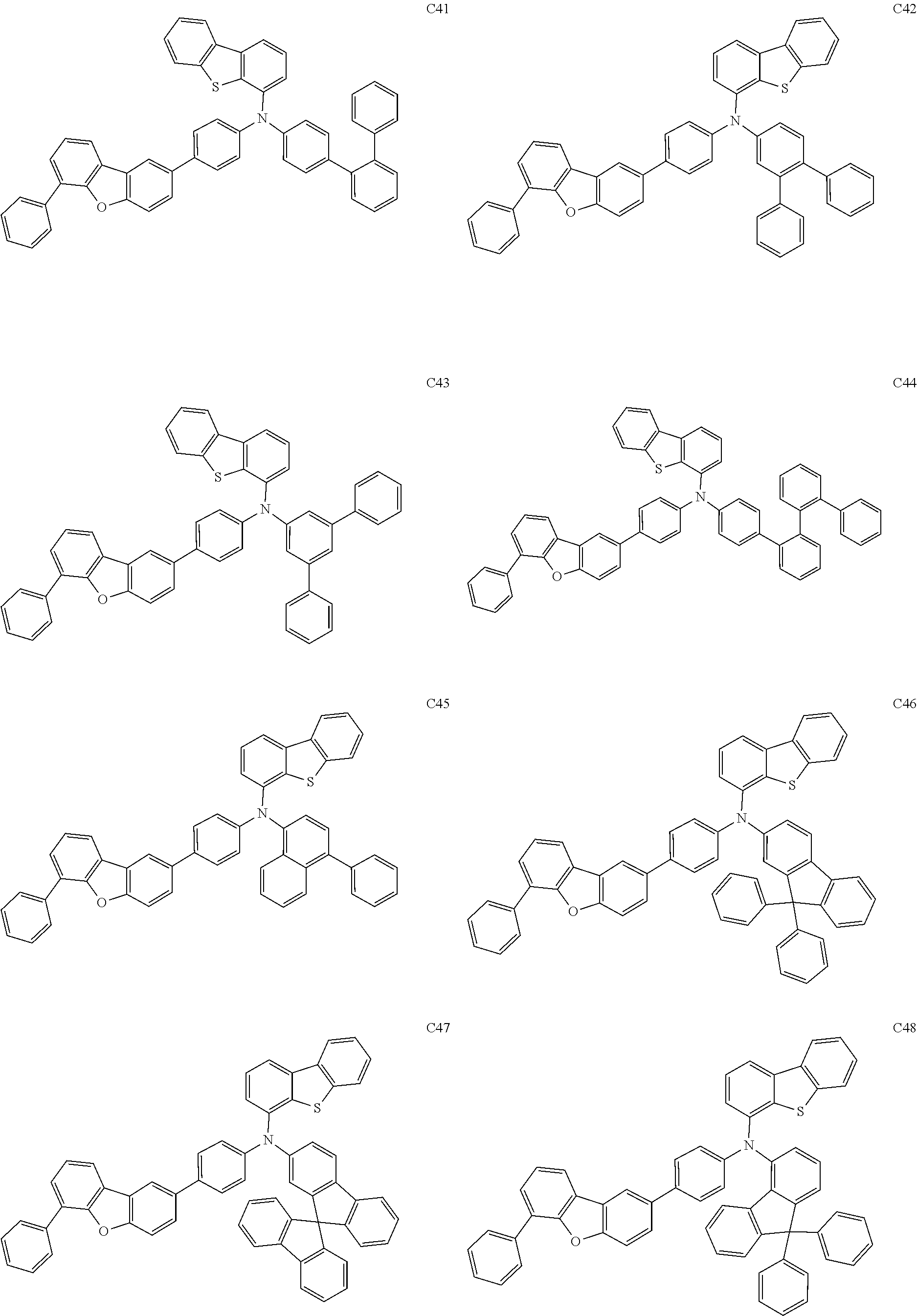

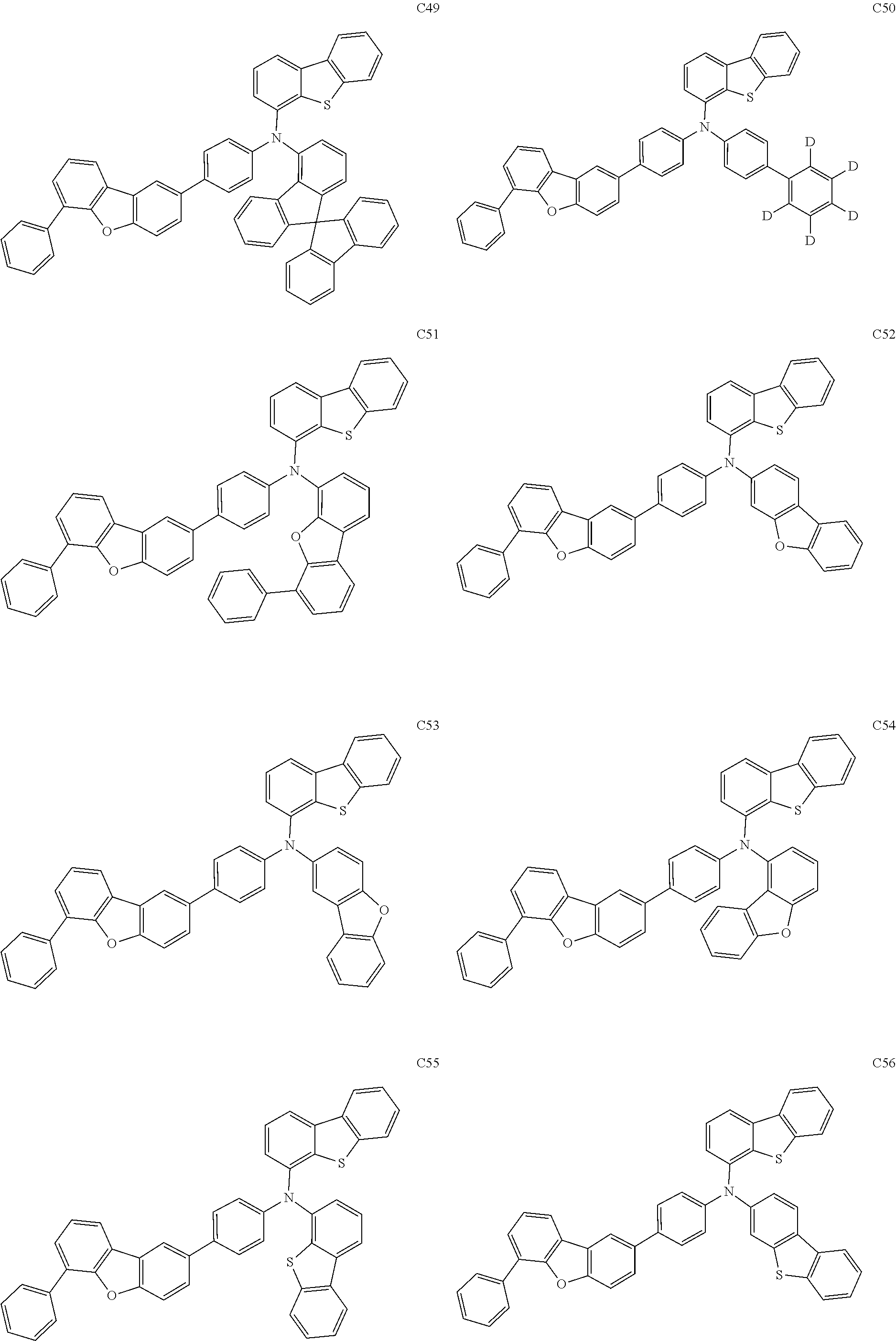

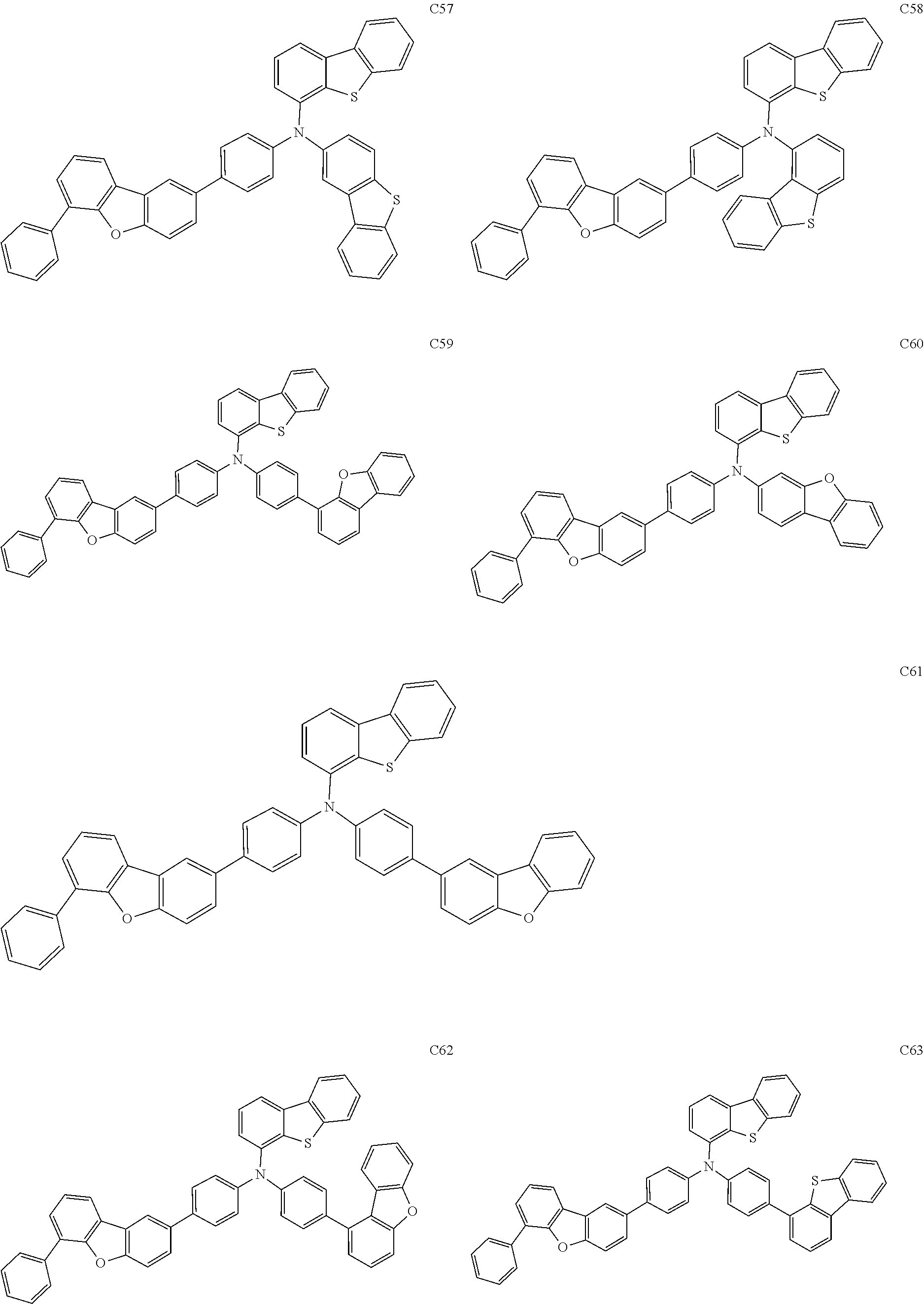

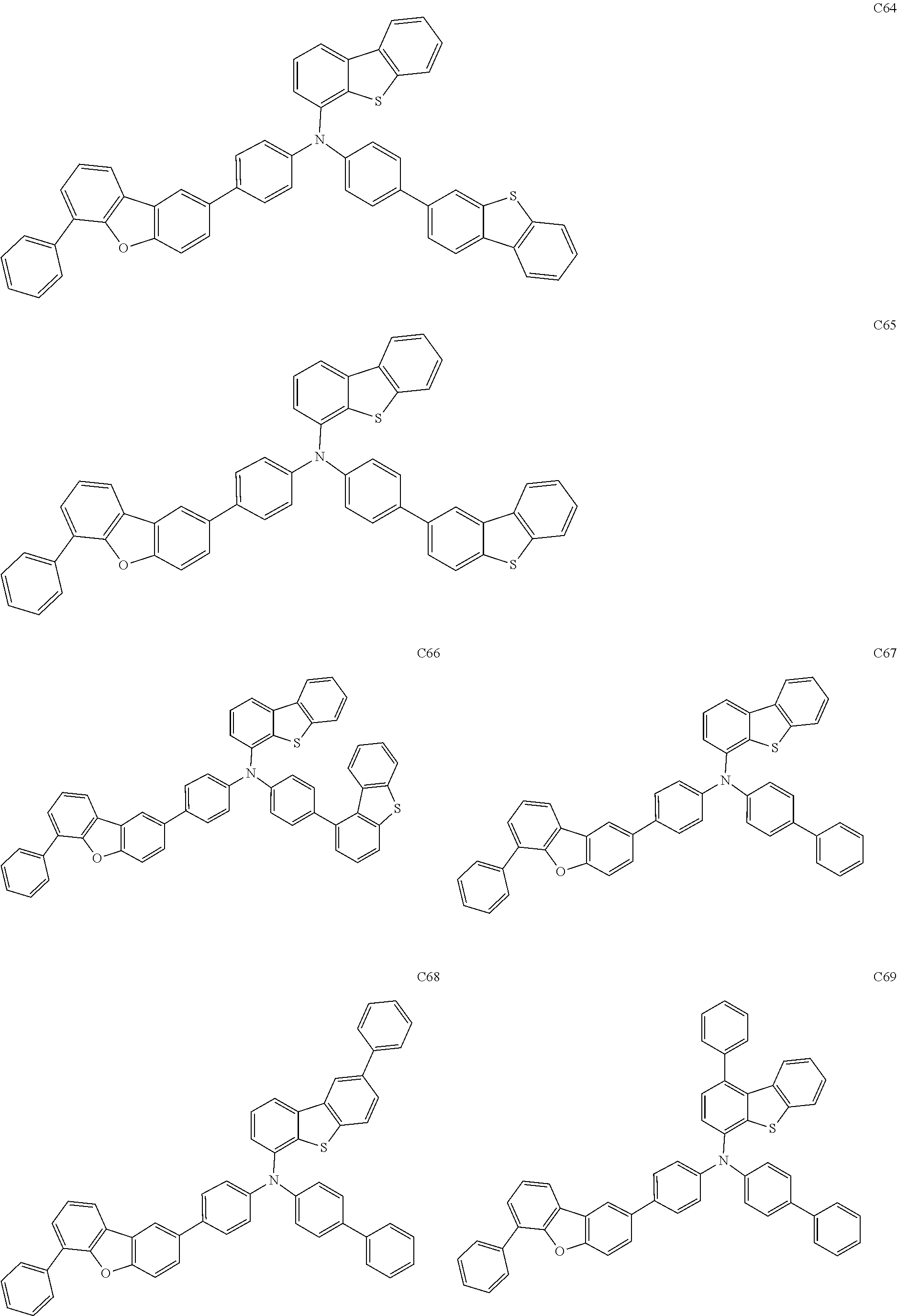

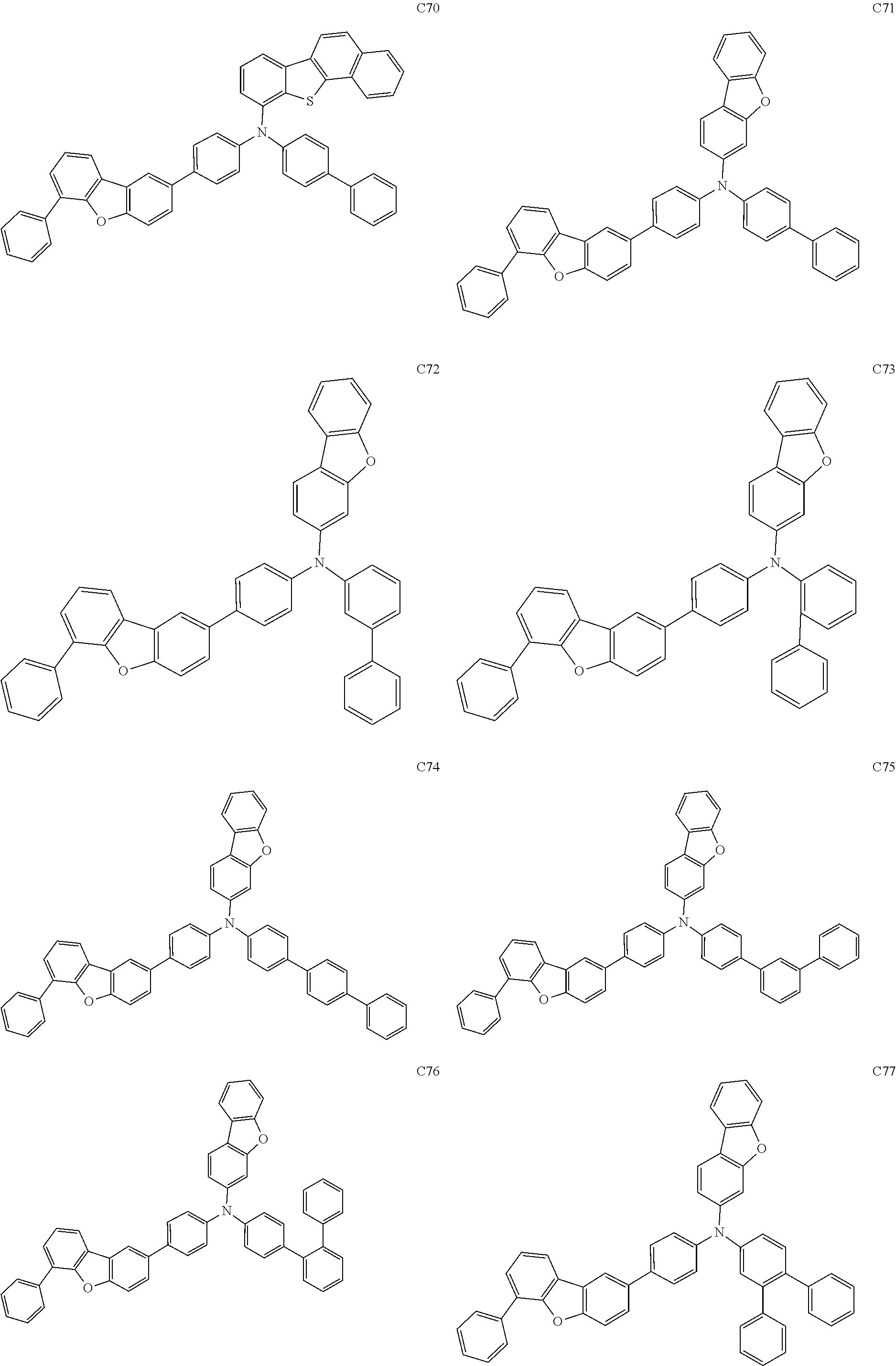

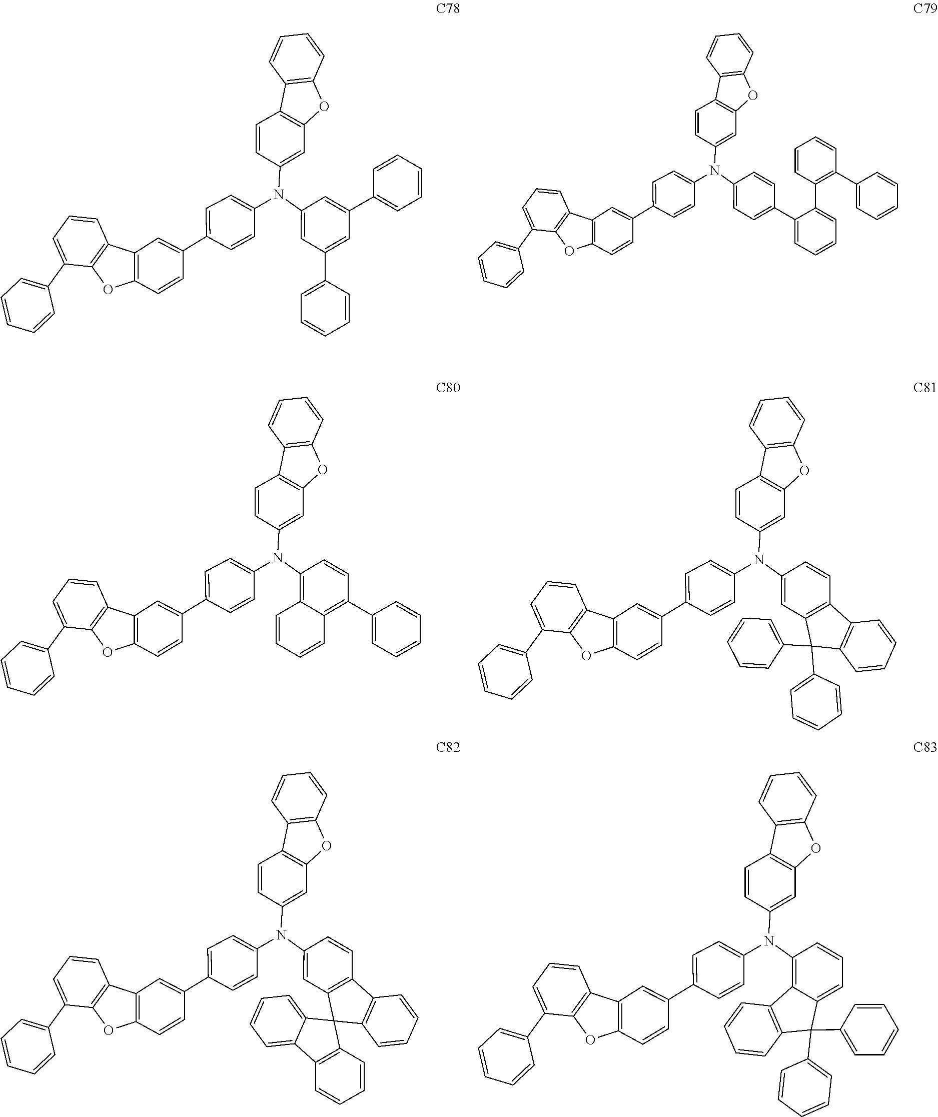

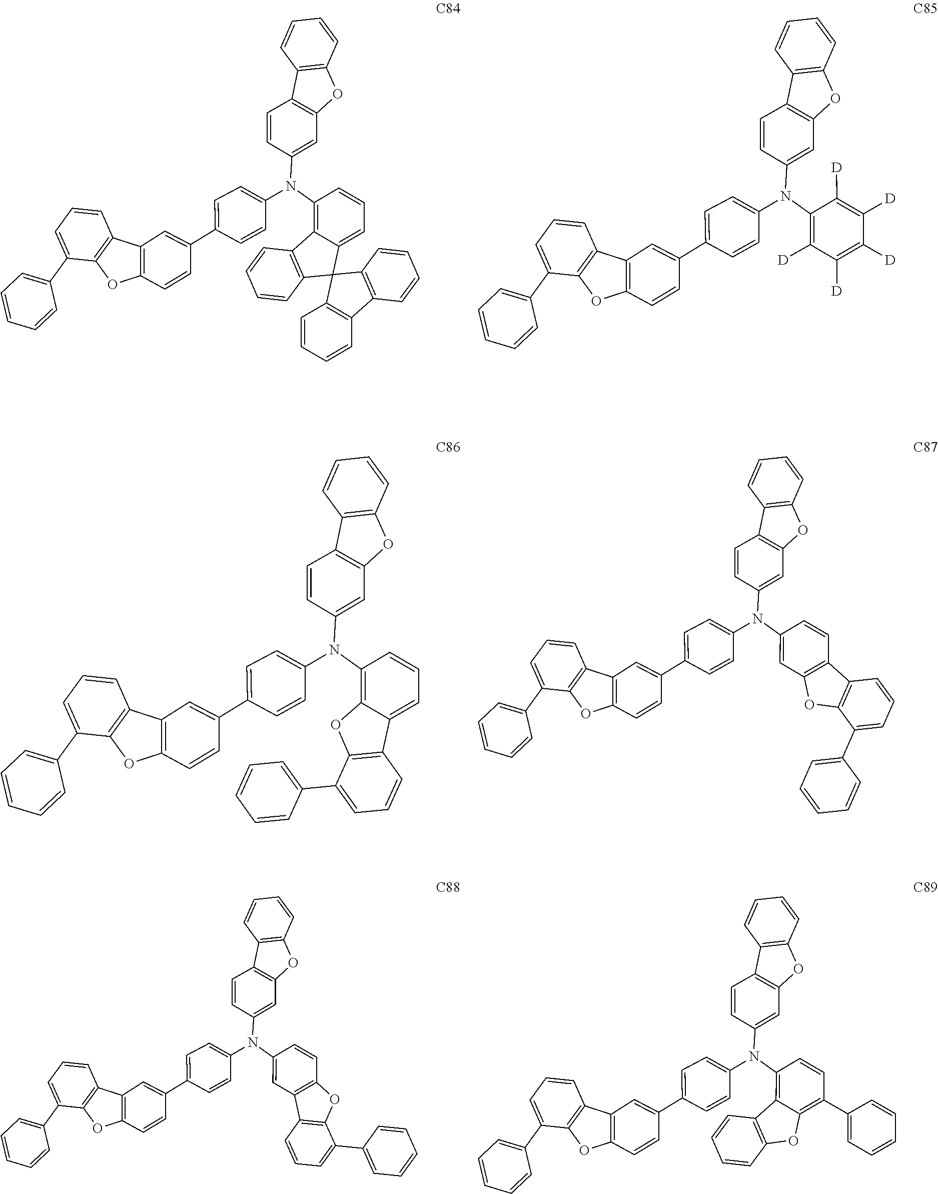

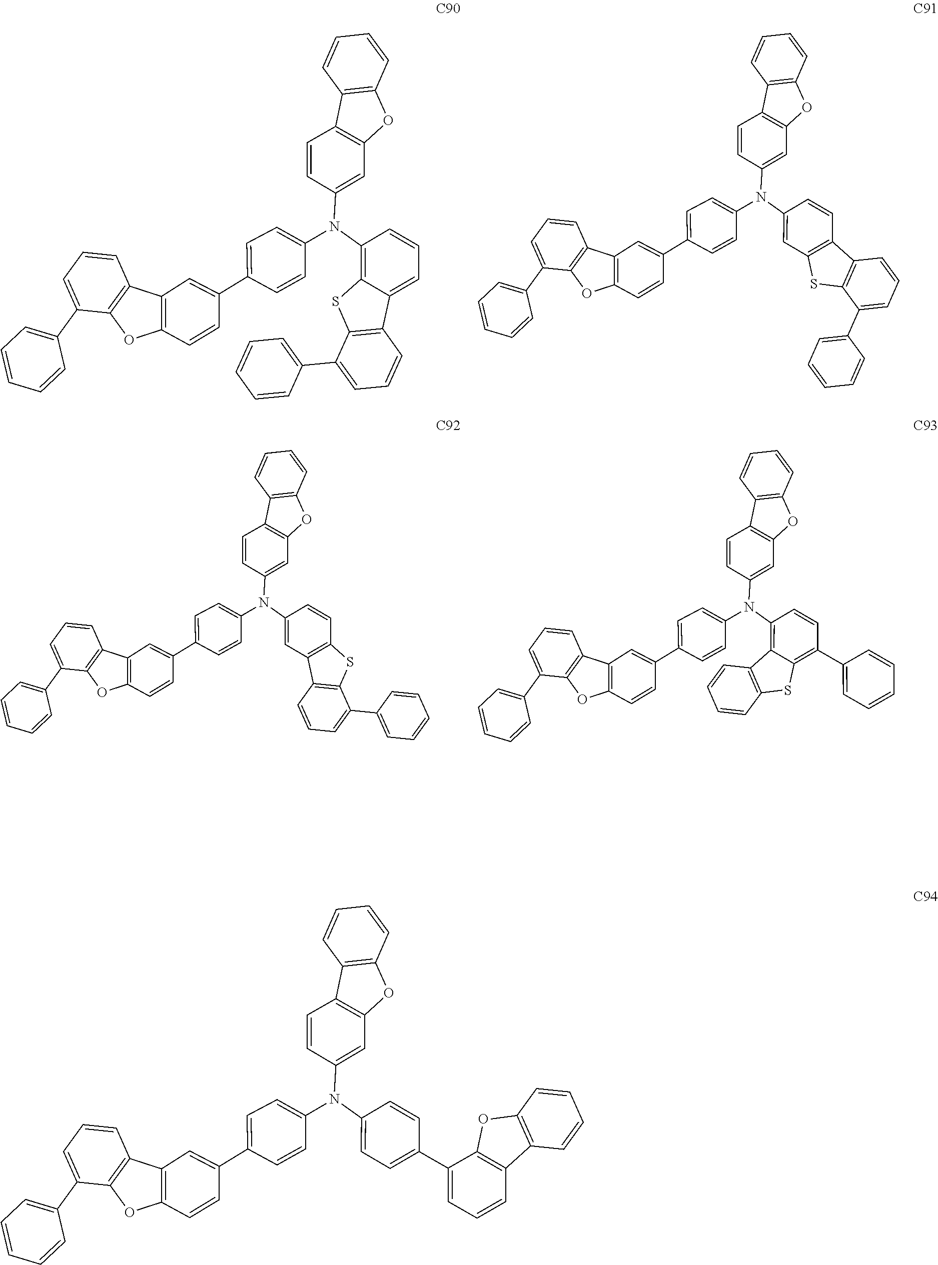 [SMILES: c1ccc(-c2cccc3c2oc2ccc(-c4ccc(N(c5ccc6c(c5)oc5ccccc56)c5cccc6c5sc5c(-c7ccccc7)cccc56)cc4)cc23)cc1 ; c1ccc(-c2cccc3c2oc2ccc(-c4ccc(N(c5ccc6c(c5)oc5ccccc56)c5ccc6c(c5)sc5c(-c7ccccc7)cccc56)cc4)cc23)cc1 ; c1ccc(-c2cccc3c2oc2ccc(-c4ccc(N(c5ccc6c(c5)oc5ccccc56)c5ccc6sc7c(-c8ccccc8)cccc7c6c5)cc4)cc23)cc1 ; c1ccc(-c2cccc3c2oc2ccc(-c4ccc(N(c5ccc6c(c5)oc5ccccc56)c5ccc(-c6ccccc6)c6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc6c(c5)oc5ccccc56)cc4)cc23)cc1]

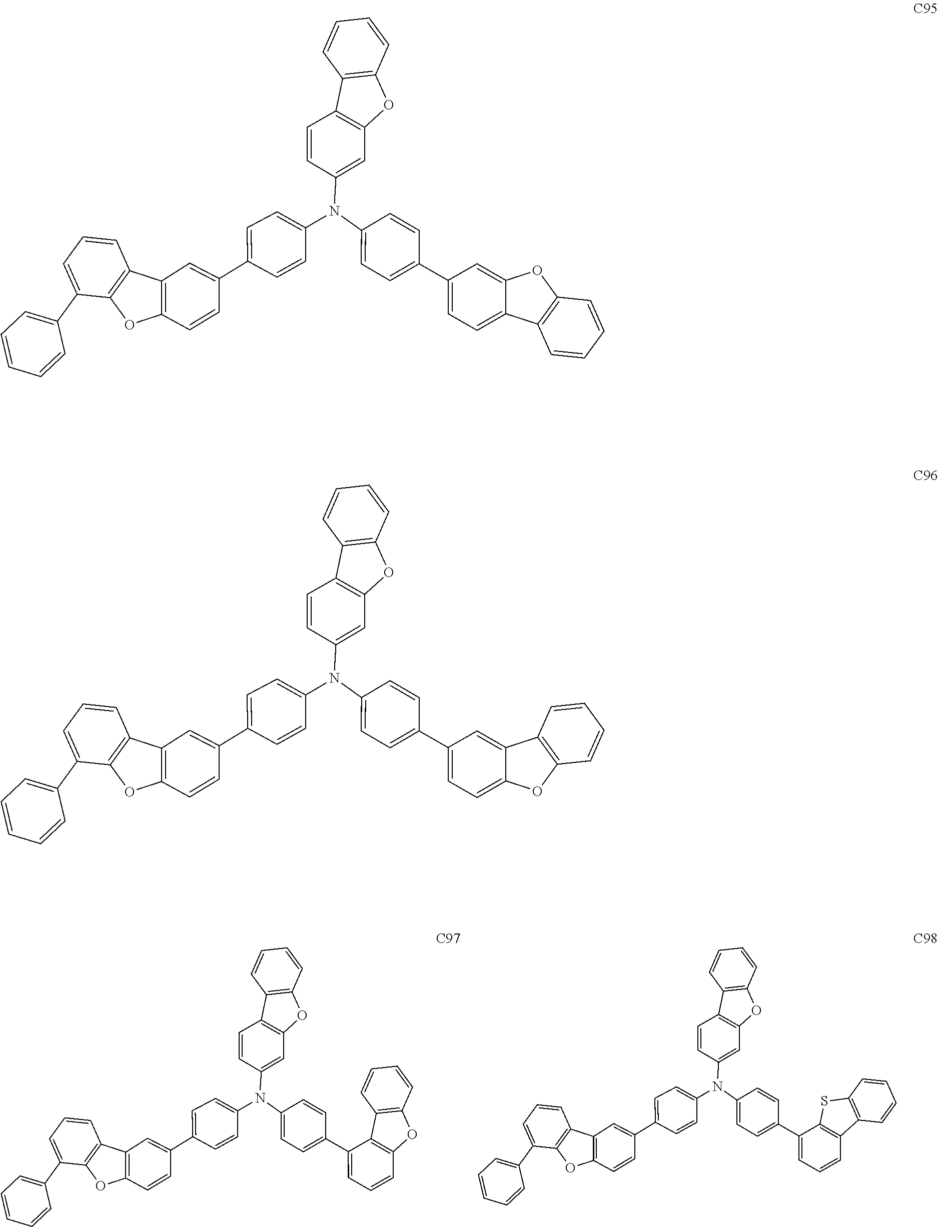 [SMILES: c1ccc(-c2cccc3c2oc2ccc(-c4ccc(N(c5ccc(-c6ccc7c(c6)oc6ccccc67)cc5)c5ccc6c(c5)oc5ccccc56)cc4)cc23)cc1 ; c1ccc(-c2cccc3c2oc2ccc(-c4ccc(N(c5ccc(-c6ccc7oc8ccccc8c7c6)cc5)c5ccc6c(c5)oc5ccccc56)cc4)cc23)cc1 ; c1ccc(-c2cccc3c2oc2ccc(-c4ccc(N(c5ccc(-c6cccc7oc8ccccc8c67)cc5)c5ccc6c(c5)oc5ccccc56)cc4)cc23)cc1 ; c1ccc(-c2cccc3c2oc2ccc(-c4ccc(N(c5ccc(-c6cccc7c6sc6ccccc67)cc5)c5ccc6c(c5)oc5ccccc56)cc4)cc23)cc1]

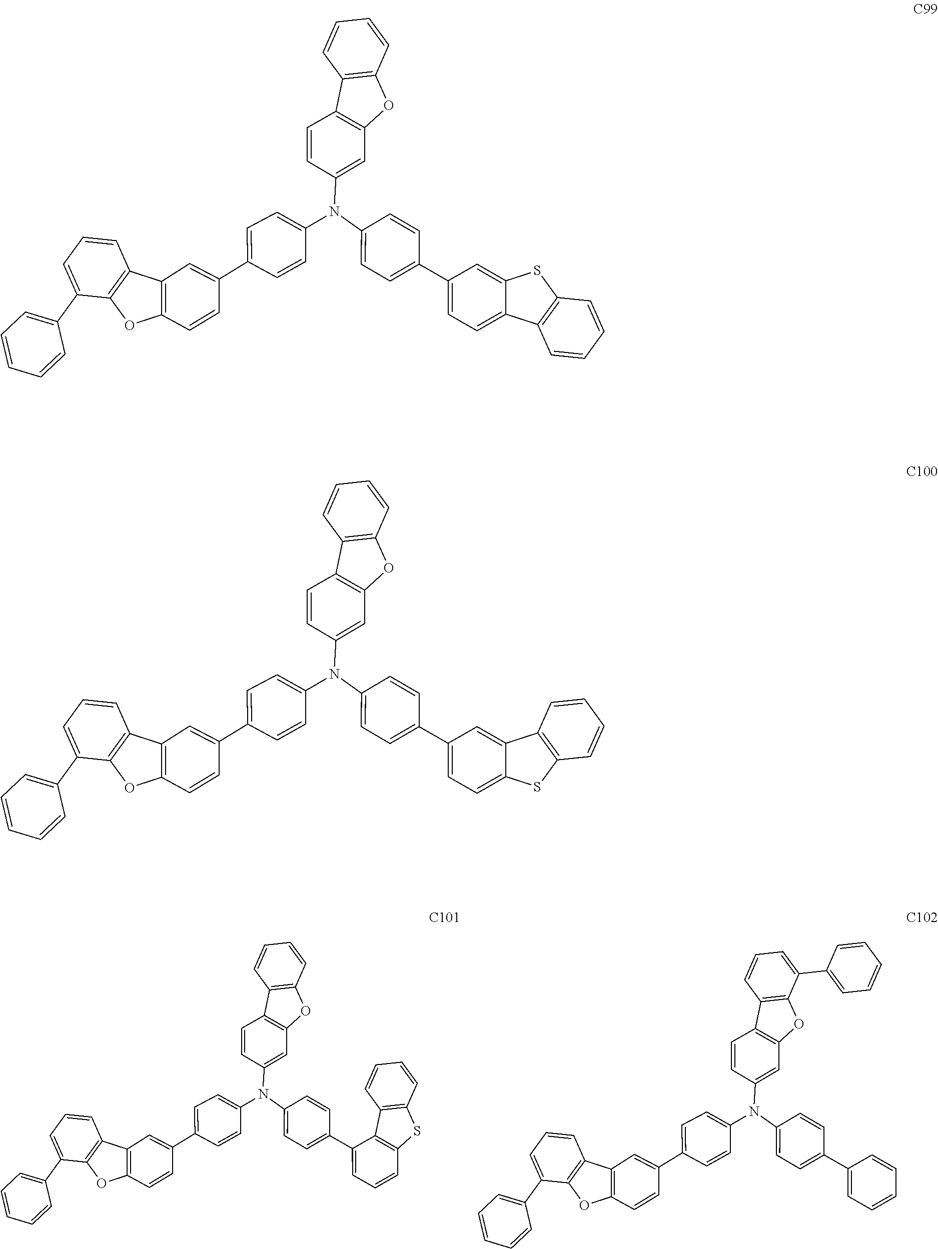 [SMILES: c1ccc(-c2cccc3c2oc2ccc(-c4ccc(N(c5ccc(-c6ccc7c(c6)sc6ccccc67)cc5)c5ccc6c(c5)oc5ccccc56)cc4)cc23)cc1 ; c1ccc(-c2cccc3c2oc2ccc(-c4ccc(N(c5ccc(-c6ccc7sc8ccccc8c7c6)cc5)c5ccc6c(c5)oc5ccccc56)cc4)cc23)cc1 ; c1ccc(-c2cccc3c2oc2ccc(-c4ccc(N(c5ccc(-c6cccc7sc8ccccc8c67)cc5)c5ccc6c(c5)oc5ccccc56)cc4)cc23)cc1 ; c1ccc(-c2ccc(N(c3ccc(-c4ccc5oc6c(-c7ccccc7)cccc6c5c4)cc3)c3ccc4c(c3)oc3c(-c5ccccc5)cccc34)cc2)cc1]

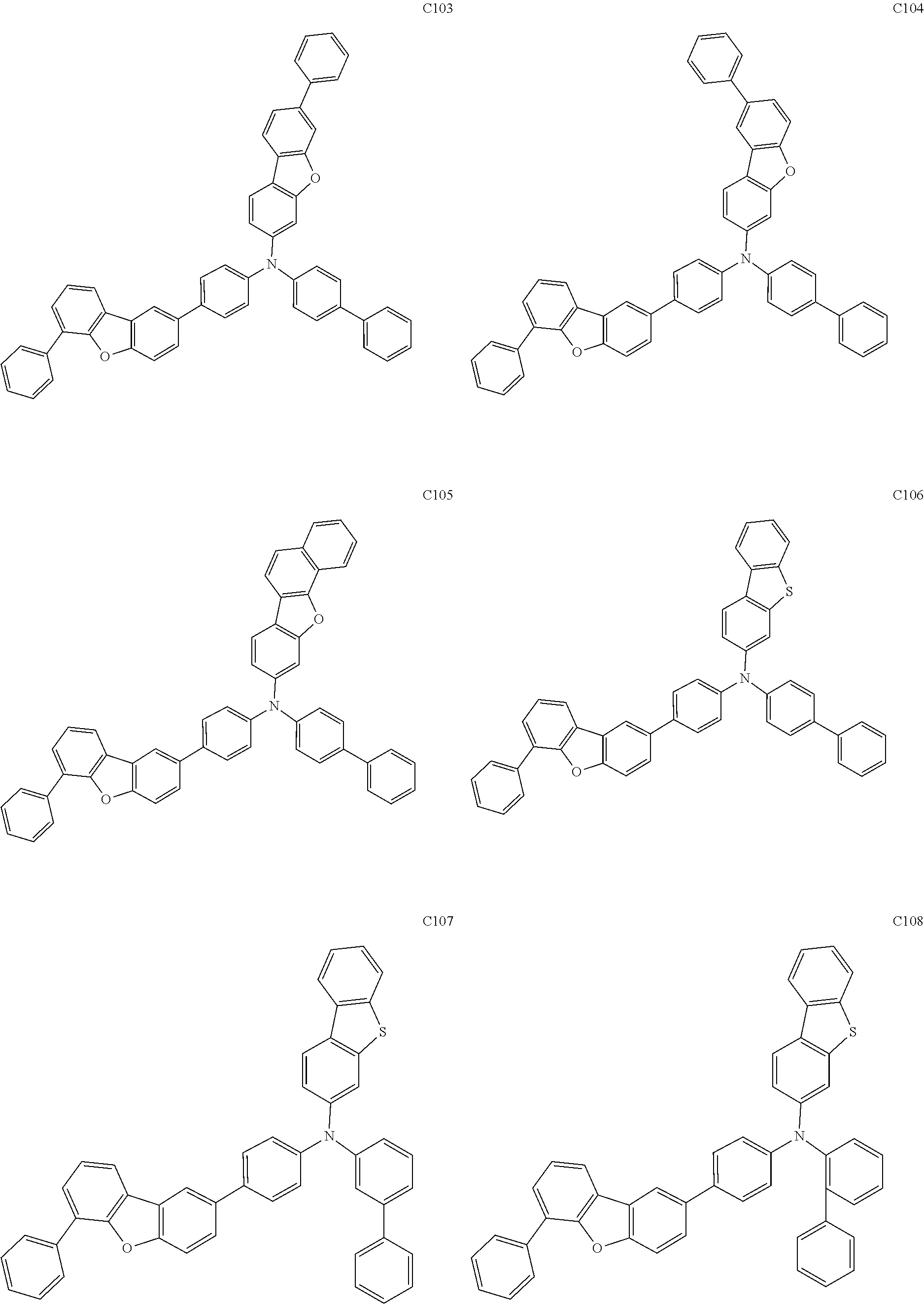

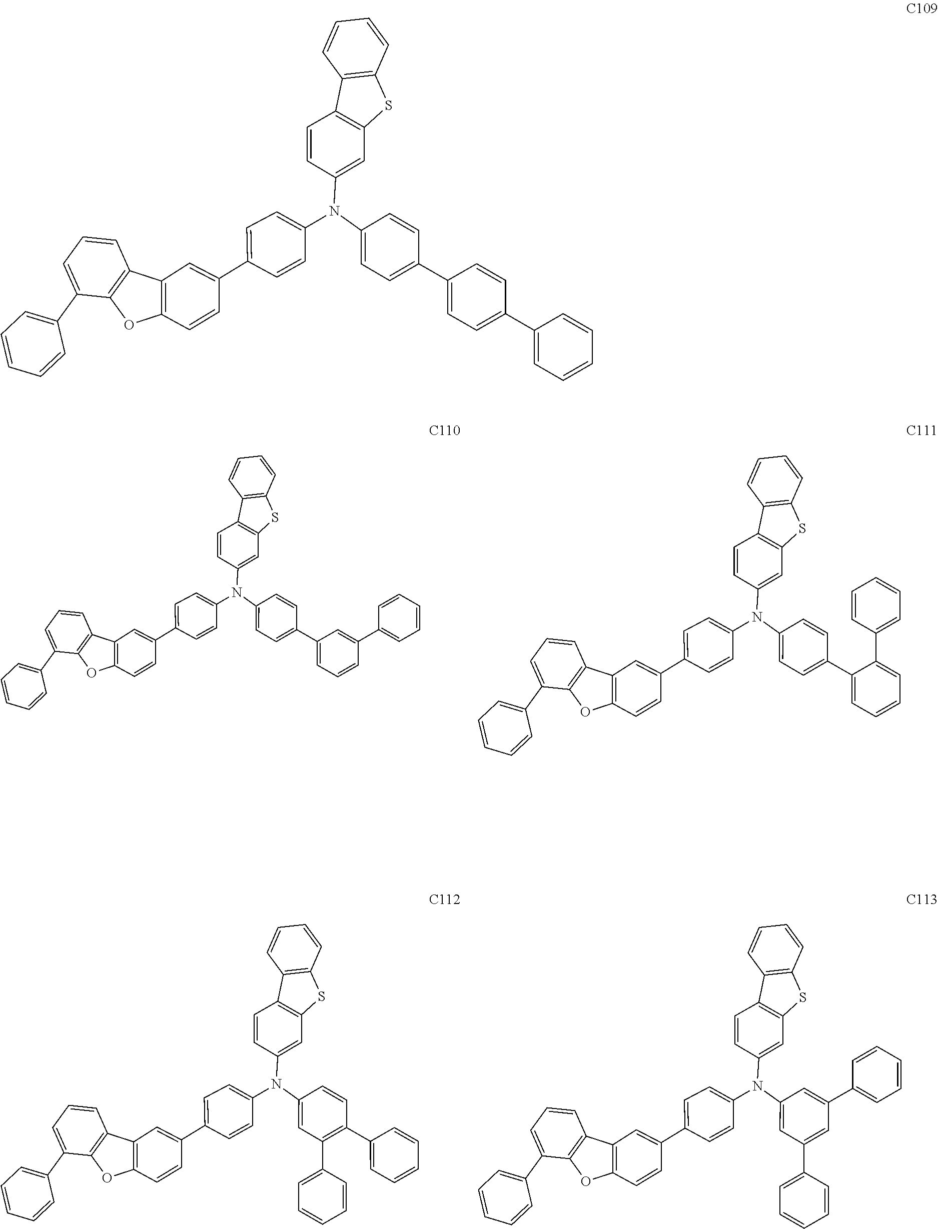 [SMILES: c1ccc(-c2ccc(-c3ccc(N(c4ccc(-c5ccc6oc7c(-c8ccccc8)cccc7c6c5)cc4)c4ccc5c(c4)sc4ccccc45)cc3)cc2)cc1 ; c1ccc(-c2cccc(-c3ccc(N(c4ccc(-c5ccc6oc7c(-c8ccccc8)cccc7c6c5)cc4)c4ccc5c(c4)sc4ccccc45)cc3)c2)cc1 ; c1ccc(-c2ccccc2-c2ccc(N(c3ccc(-c4ccc5oc6c(-c7ccccc7)cccc6c5c4)cc3)c3ccc4c(c3)sc3ccccc34)cc2)cc1 ; c1ccc(-c2ccc(N(c3ccc(-c4ccc5oc6c(-c7ccccc7)cccc6c5c4)cc3)c3ccc4c(c3)sc3ccccc34)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc4c(c3)sc3ccccc34)c2)cc1]

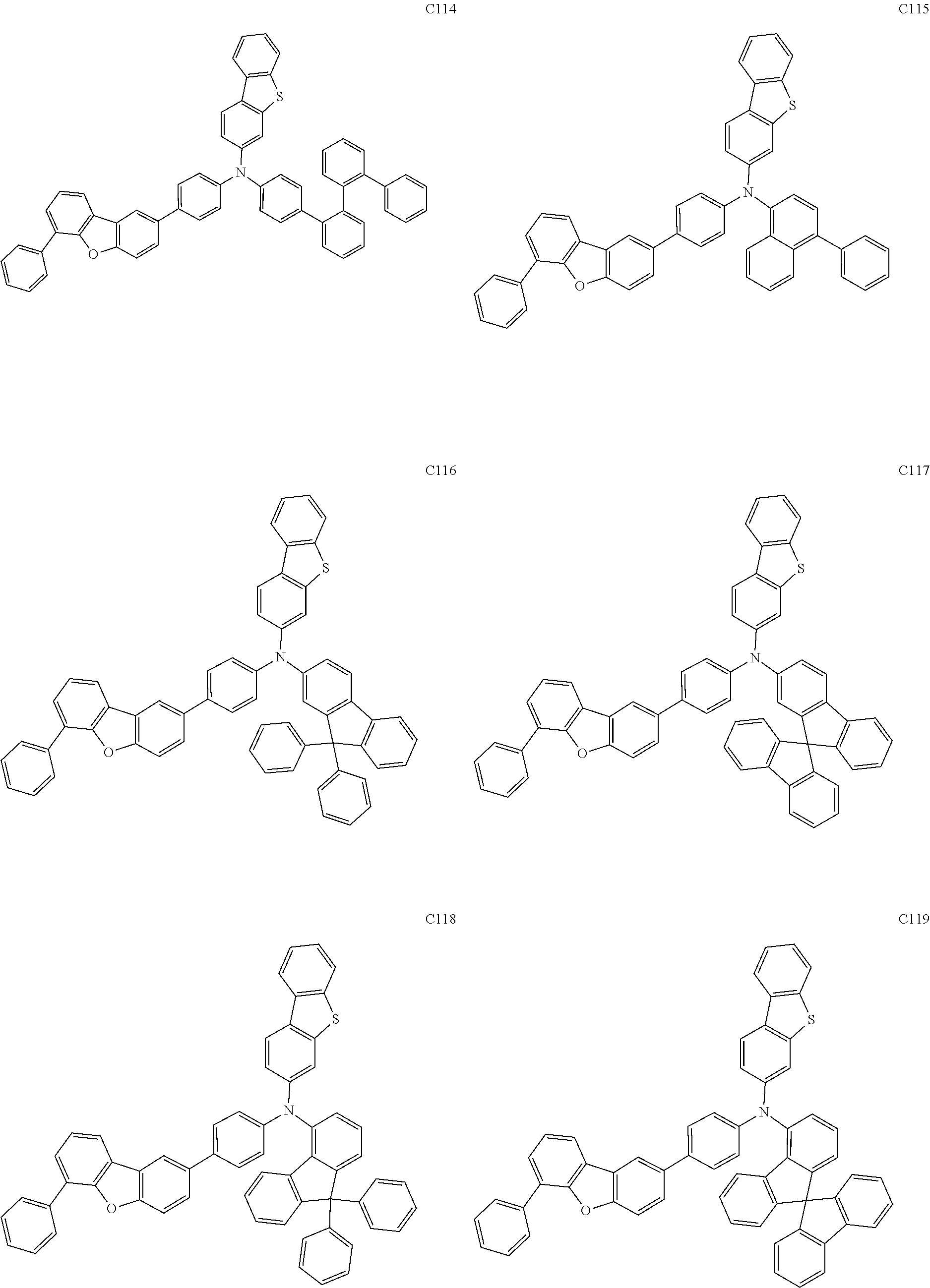

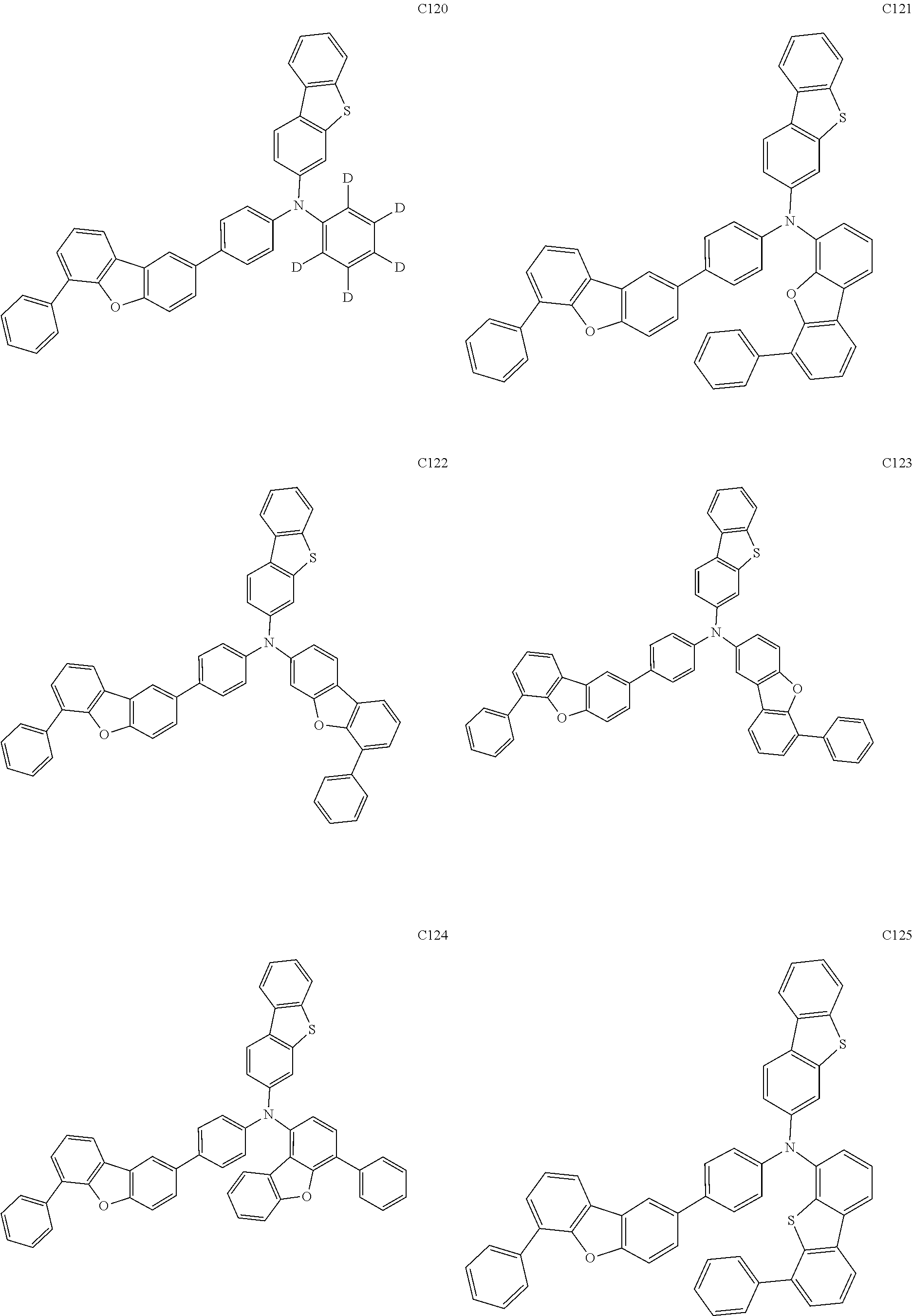

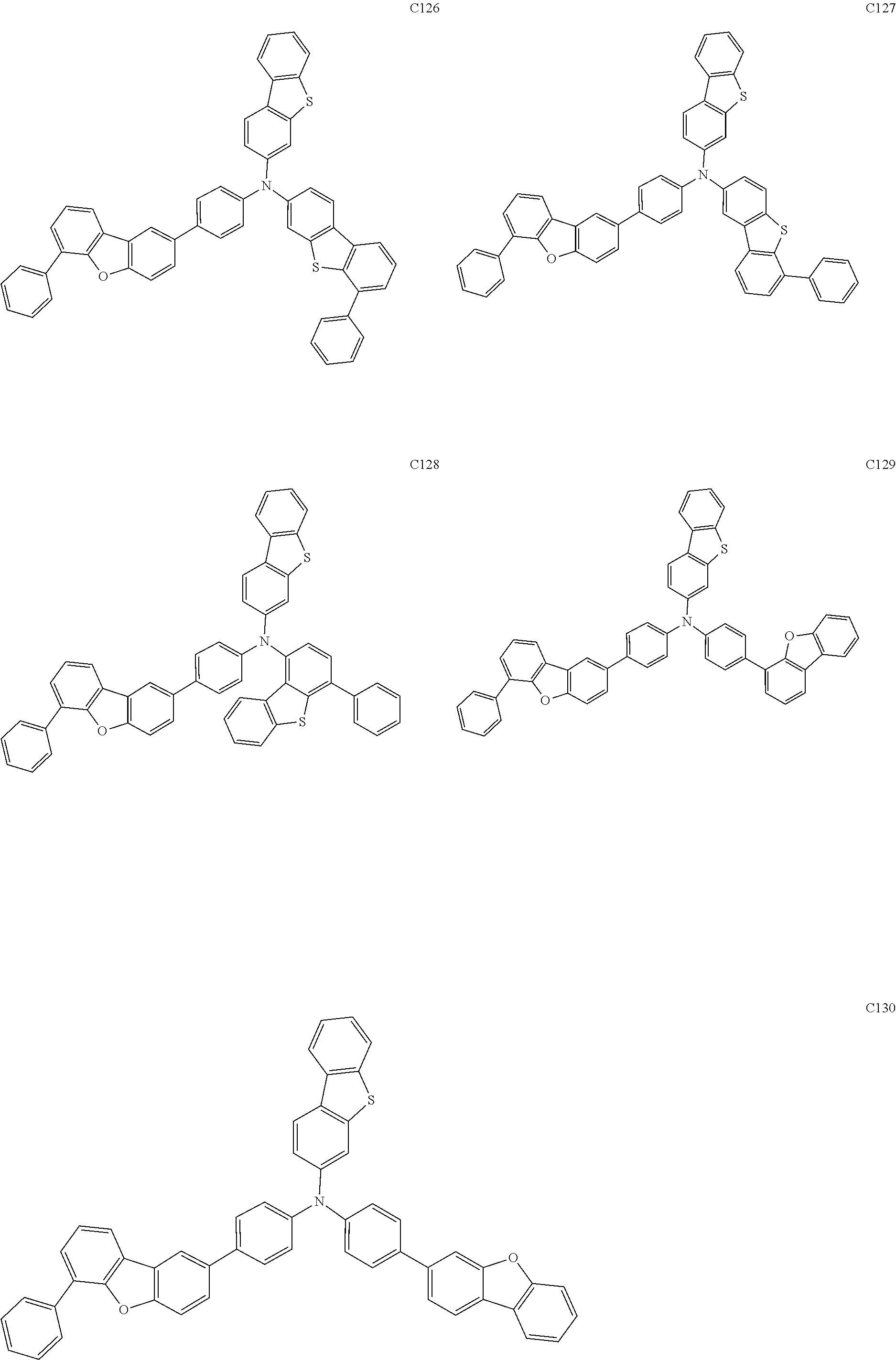

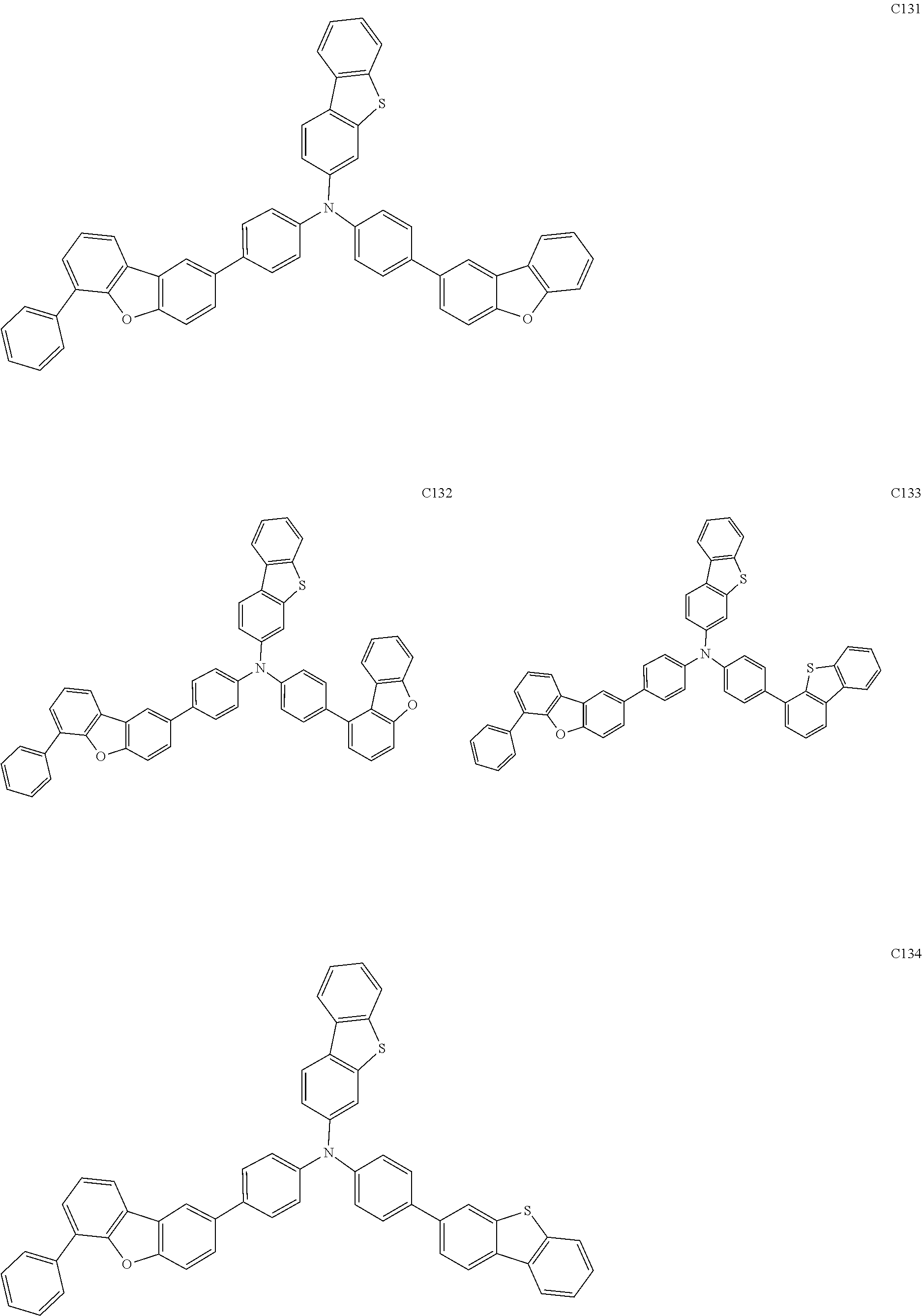 [SMILES: c1ccc(-c2cccc3c2oc2ccc(-c4ccc(N(c5ccc(-c6ccc7oc8ccccc8c7c6)cc5)c5ccc6c(c5)sc5ccccc56)cc4)cc23)cc1 ; c1ccc(-c2cccc3c2oc2ccc(-c4ccc(N(c5ccc(-c6cccc7oc8ccccc8c67)cc5)c5ccc6c(c5)sc5ccccc56)cc4)cc23)cc1 ; c1ccc(-c2cccc3c2oc2ccc(-c4ccc(N(c5ccc(-c6cccc7c6sc6ccccc67)cc5)c5ccc6c(c5)sc5ccccc56)cc4)cc23)cc1 ; c1ccc(-c2cccc3c2oc2ccc(-c4ccc(N(c5ccc(-c6ccc7c(c6)sc6ccccc67)cc5)c5ccc6c(c5)sc5ccccc56)cc4)cc23)cc1]

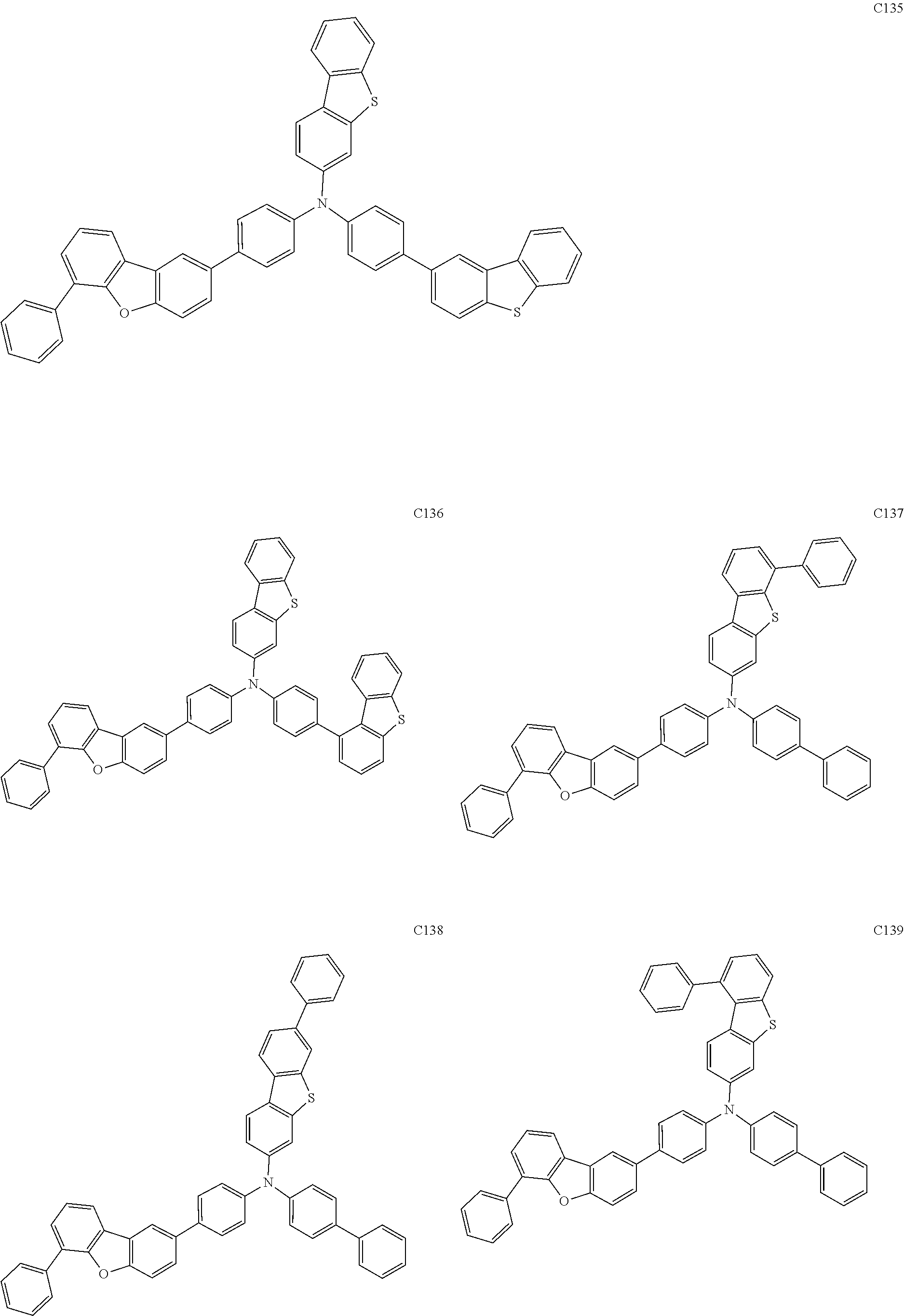 [SMILES: c1ccc(-c2cccc3c2oc2ccc(-c4ccc(N(c5ccc(-c6ccc7sc8ccccc8c7c6)cc5)c5ccc6c(c5)sc5ccccc56)cc4)cc23)cc1 ; c1ccc(-c2cccc3c2oc2ccc(-c4ccc(N(c5ccc(-c6cccc7sc8ccccc8c67)cc5)c5ccc6c(c5)sc5ccccc56)cc4)cc23)cc1 ; c1ccc(-c2ccc(N(c3ccc(-c4ccc5oc6c(-c7ccccc7)cccc6c5c4)cc3)c3ccc4c(c3)sc3c(-c5ccccc5)cccc34)cc2)cc1 ; c1ccc(-c2ccc(N(c3ccc(-c4ccc5oc6c(-c7ccccc7)cccc6c5c4)cc3)c3ccc4c(c3)sc3cc(-c5ccccc5)ccc34)cc2)cc1 ; c1ccc(-c2ccc(N(c3ccc(-c4ccc5oc6c(-c7ccccc7)cccc6c5c4)cc3)c3ccc4c(c3)sc3cccc(-c5ccccc5)c34)cc2)cc1]

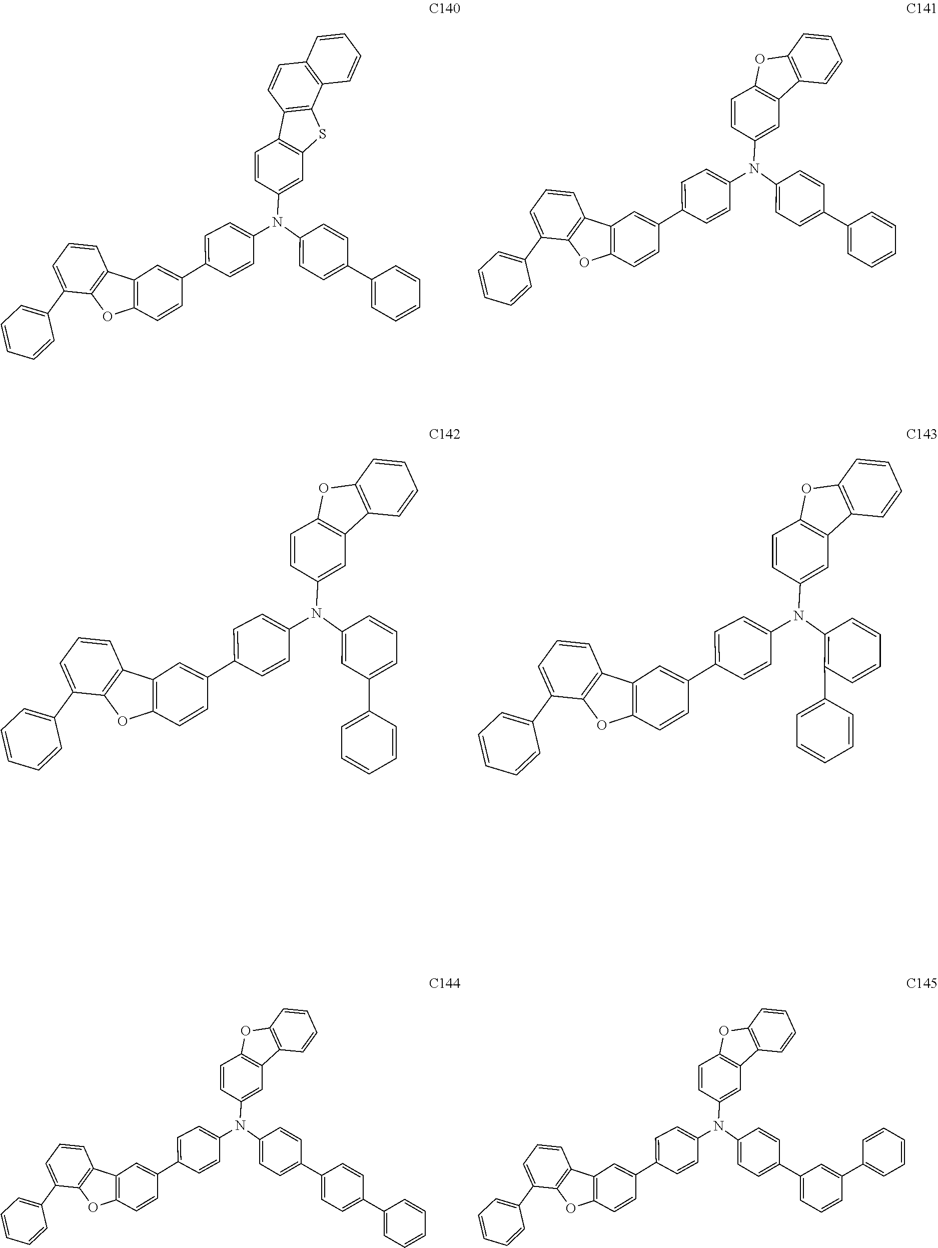

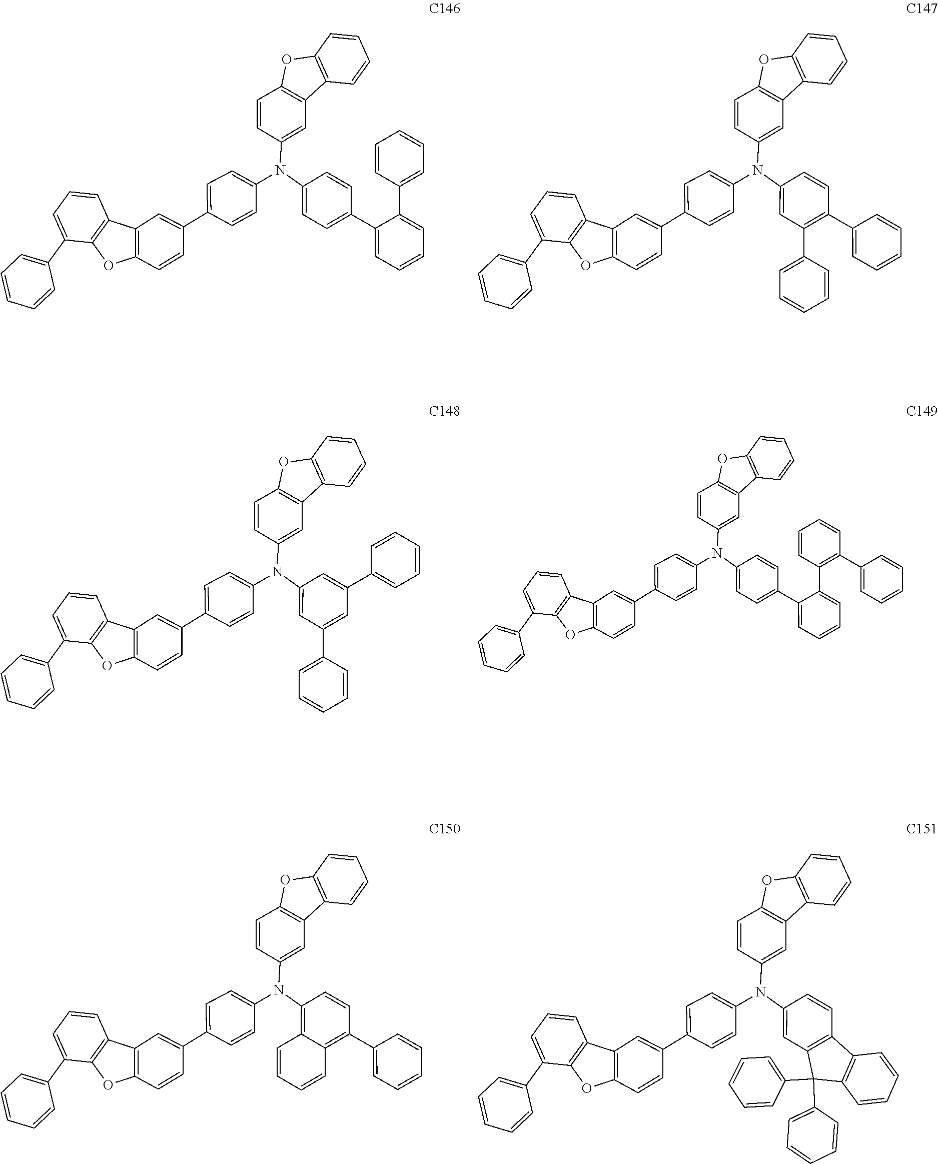

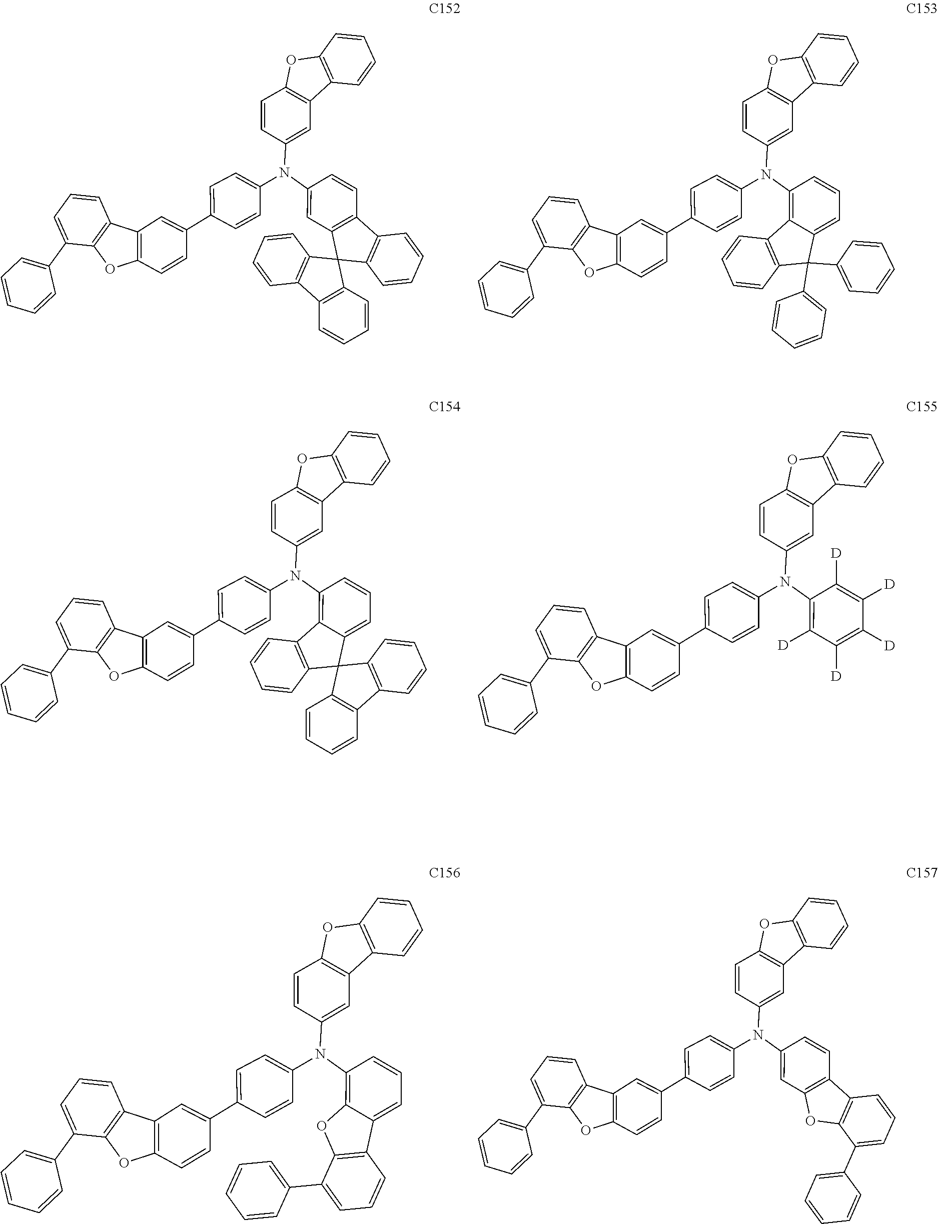

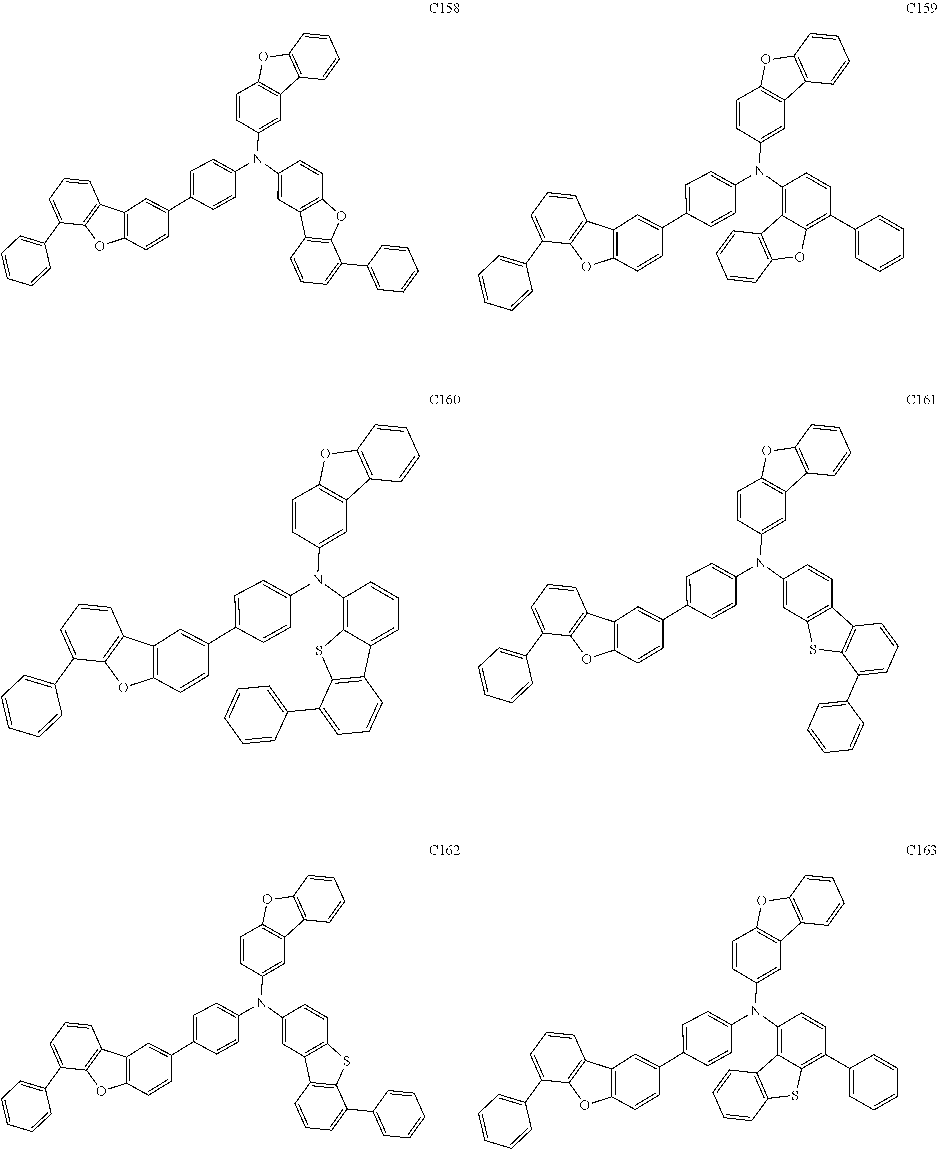

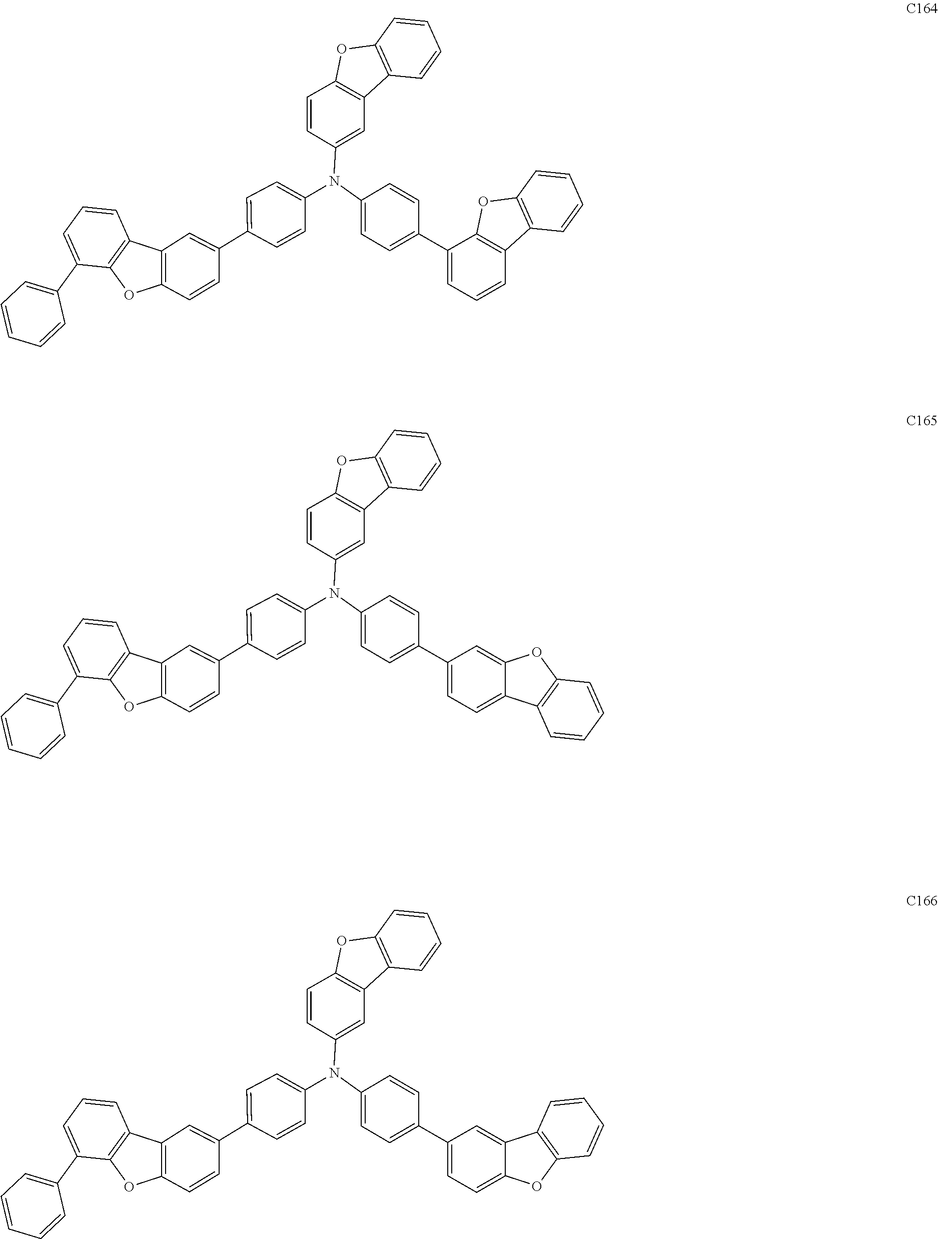 [SMILES: c1ccc(-c2cccc3c2oc2ccc(-c4ccc(N(c5ccc(-c6cccc7c6oc6ccccc67)cc5)c5ccc6oc7ccccc7c6c5)cc4)cc23)cc1 ; c1ccc(-c2cccc3c2oc2ccc(-c4ccc(N(c5ccc(-c6ccc7c(c6)oc6ccccc67)cc5)c5ccc6oc7ccccc7c6c5)cc4)cc23)cc1 ; c1ccc(-c2cccc3c2oc2ccc(-c4ccc(N(c5ccc(-c6ccc7oc8ccccc8c7c6)cc5)c5ccc6oc7ccccc7c6c5)cc4)cc23)cc1]

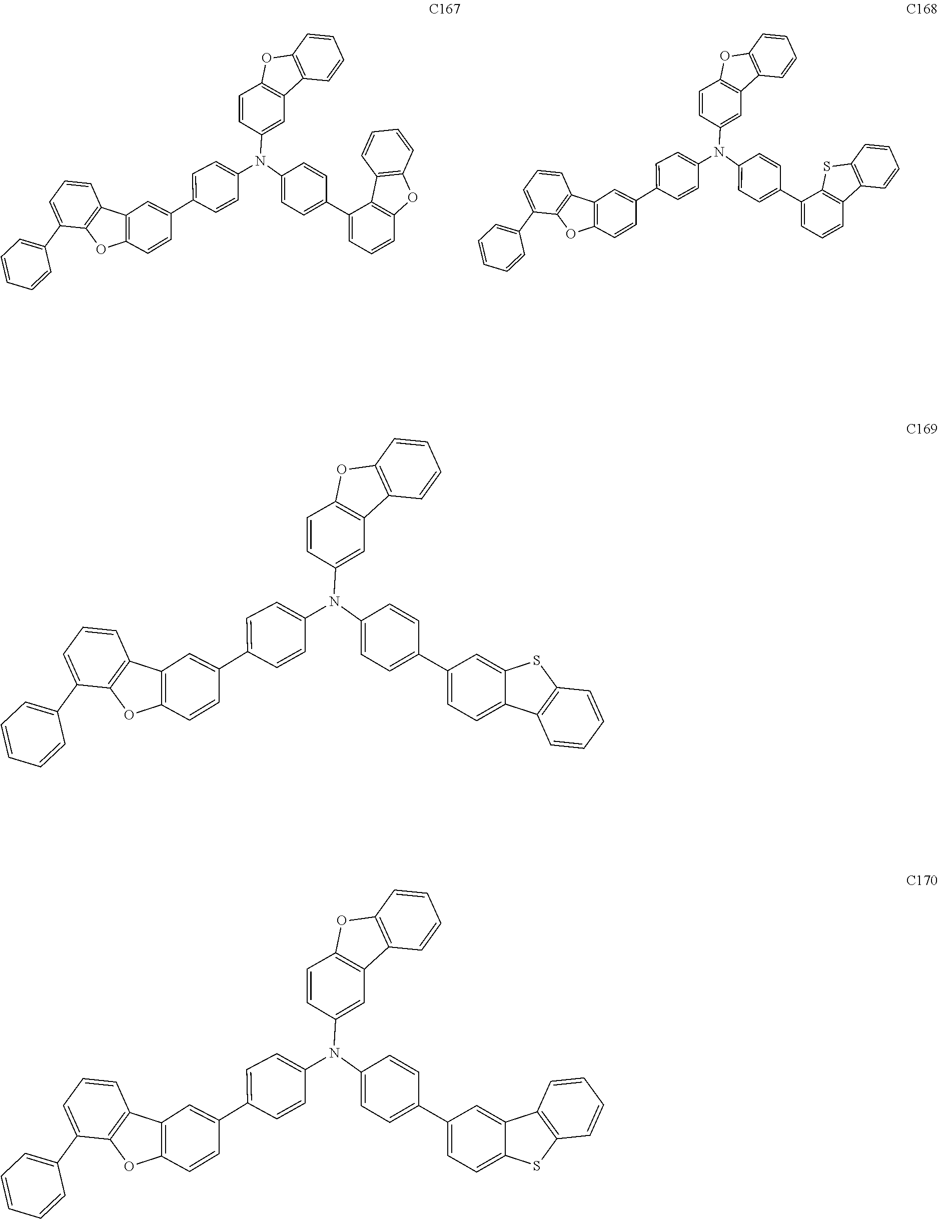 [SMILES: c1ccc(-c2cccc3c2oc2ccc(-c4ccc(N(c5ccc(-c6cccc7oc8ccccc8c67)cc5)c5ccc6oc7ccccc7c6c5)cc4)cc23)cc1 ; c1ccc(-c2cccc3c2oc2ccc(-c4ccc(N(c5ccc(-c6cccc7c6sc6ccccc67)cc5)c5ccc6oc7ccccc7c6c5)cc4)cc23)cc1 ; c1ccc(-c2cccc3c2oc2ccc(-c4ccc(N(c5ccc(-c6ccc7c(c6)sc6ccccc67)cc5)c5ccc6oc7ccccc7c6c5)cc4)cc23)cc1 ; c1ccc(-c2cccc3c2oc2ccc(-c4ccc(N(c5ccc(-c6ccc7sc8ccccc8c7c6)cc5)c5ccc6oc7ccccc7c6c5)cc4)cc23)cc1]

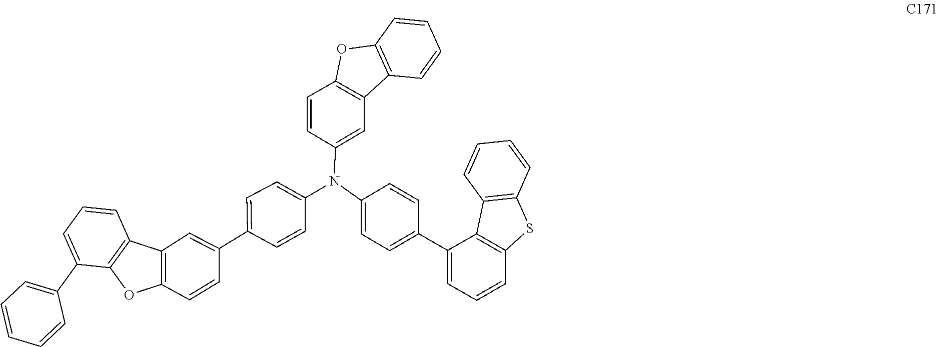 [SMILES: c1ccc(-c2cccc3c2oc2ccc(-c4ccc(N(c5ccc(-c6cccc7sc8ccccc8c67)cc5)c5ccc6oc7ccccc7c6c5)cc4)cc23)cc1]

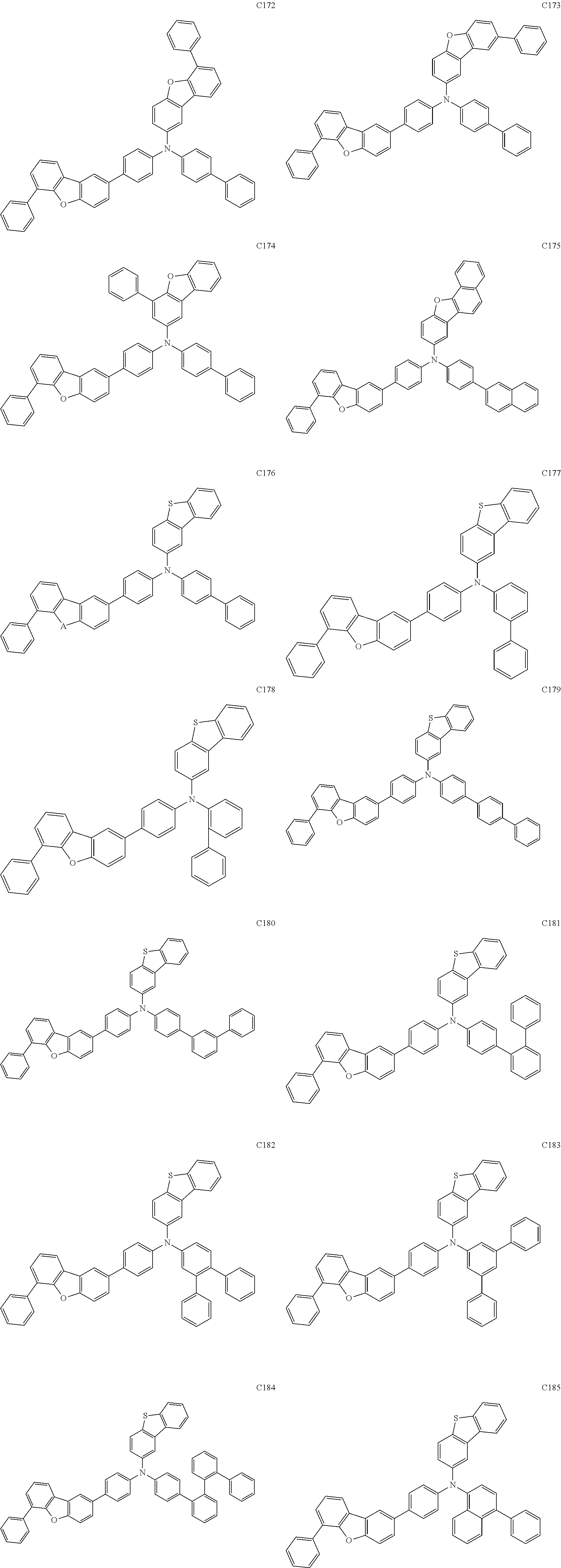

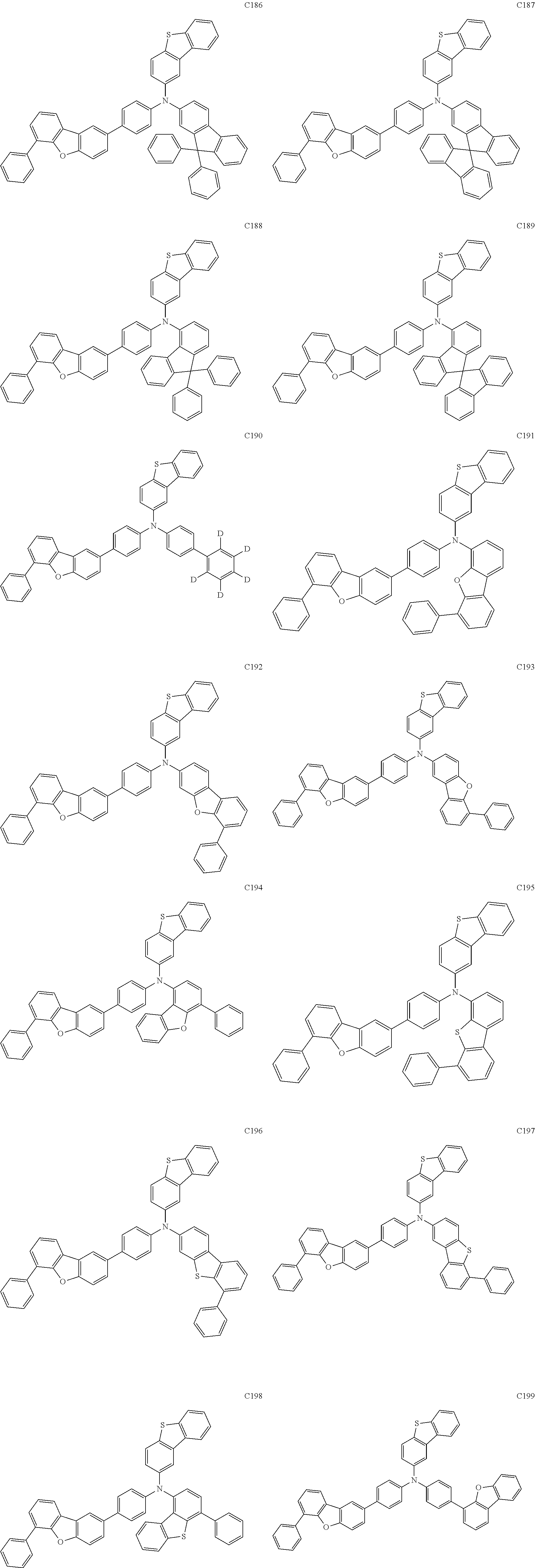

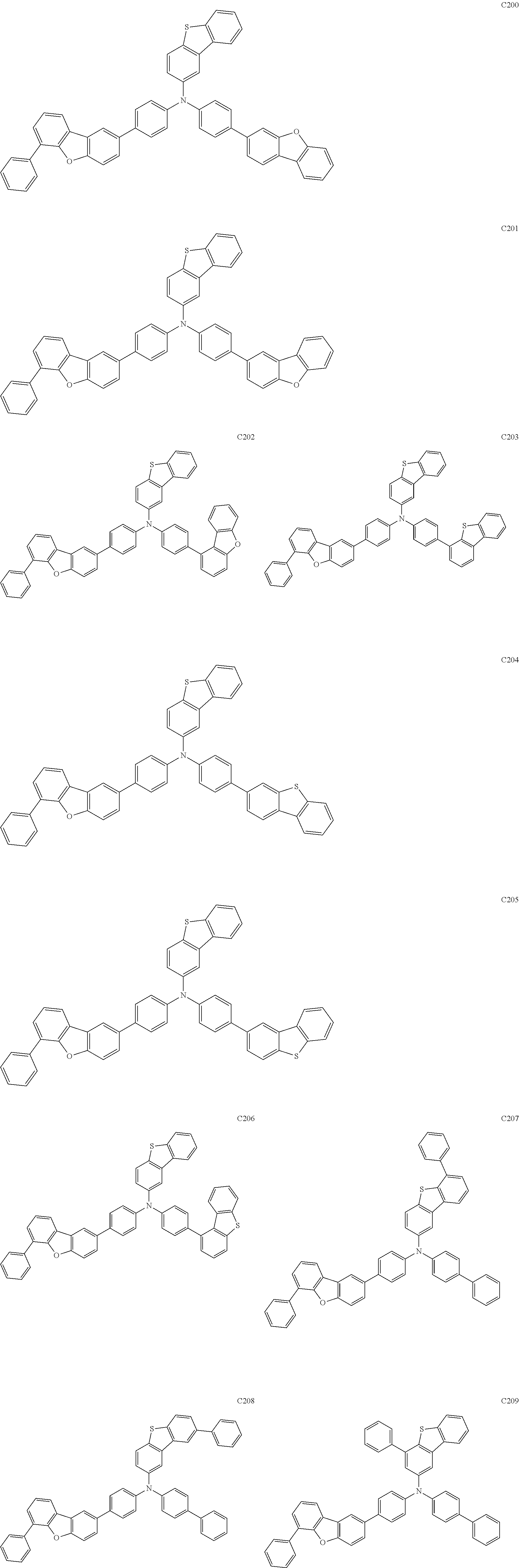

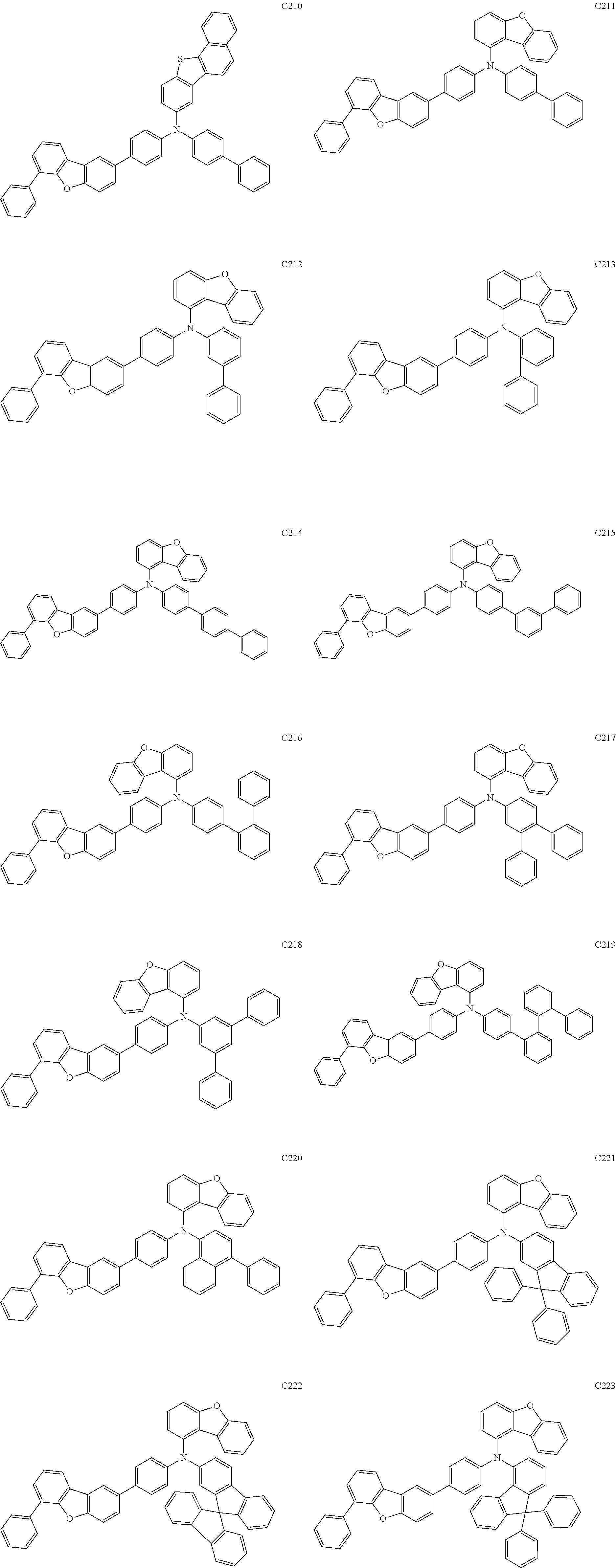

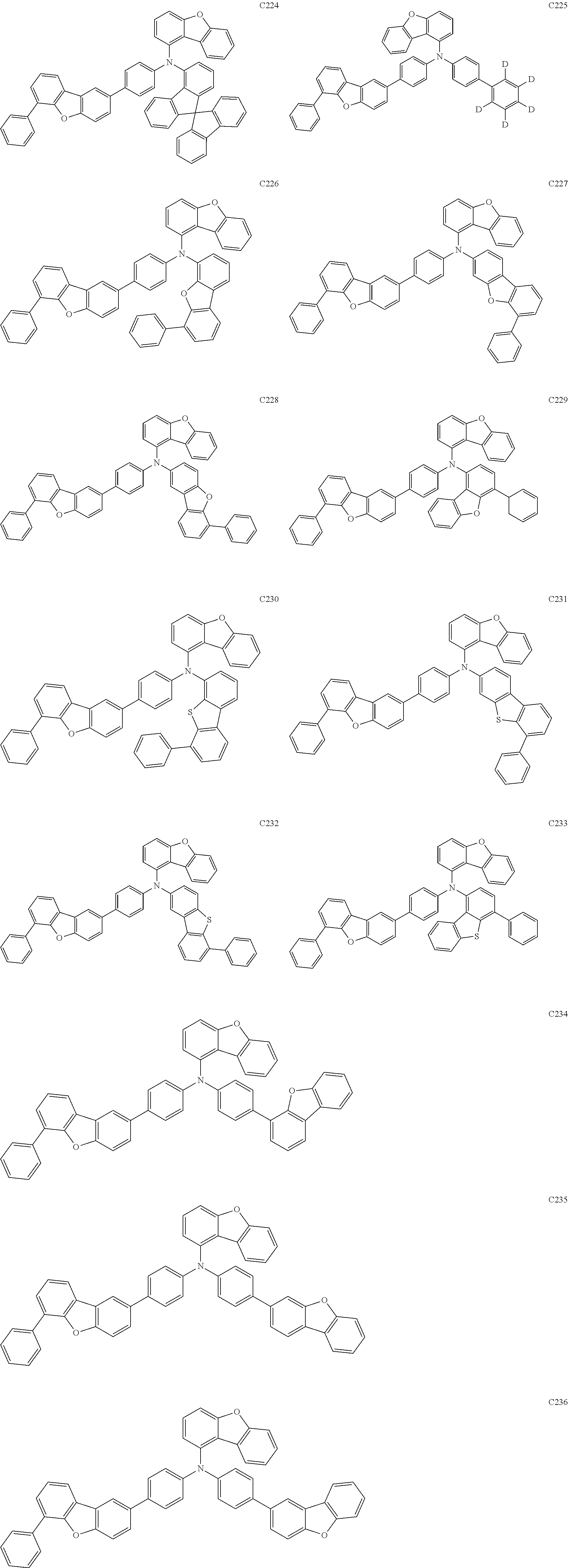

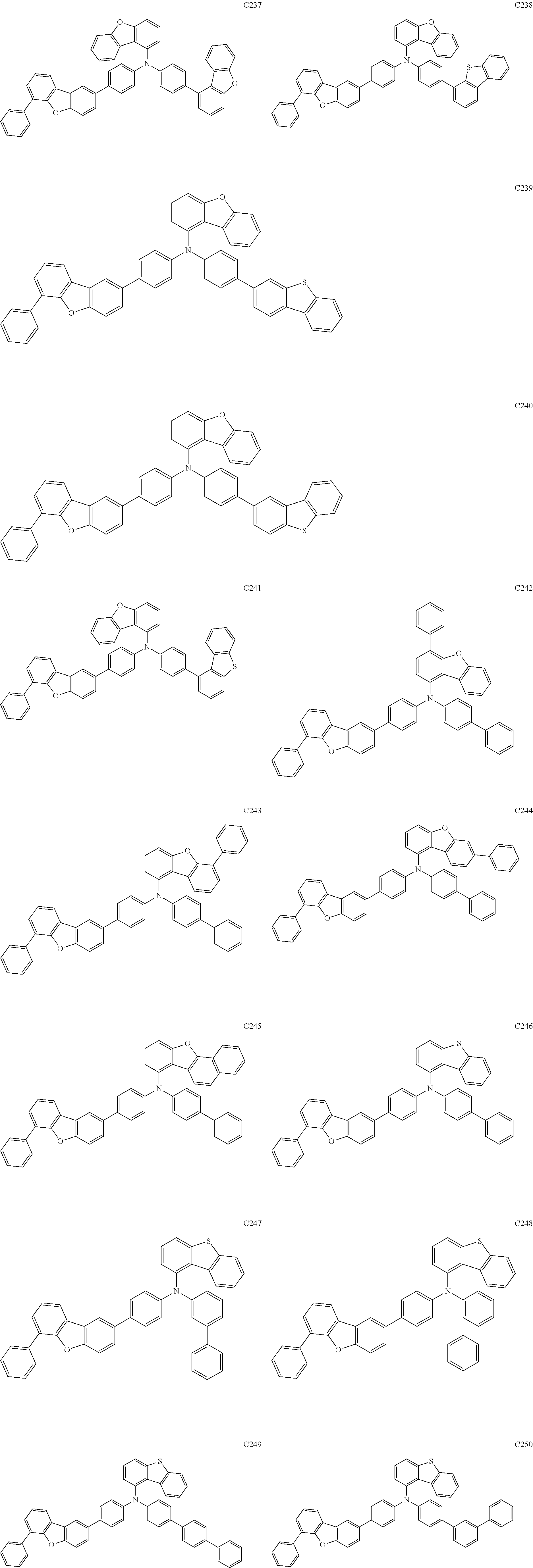

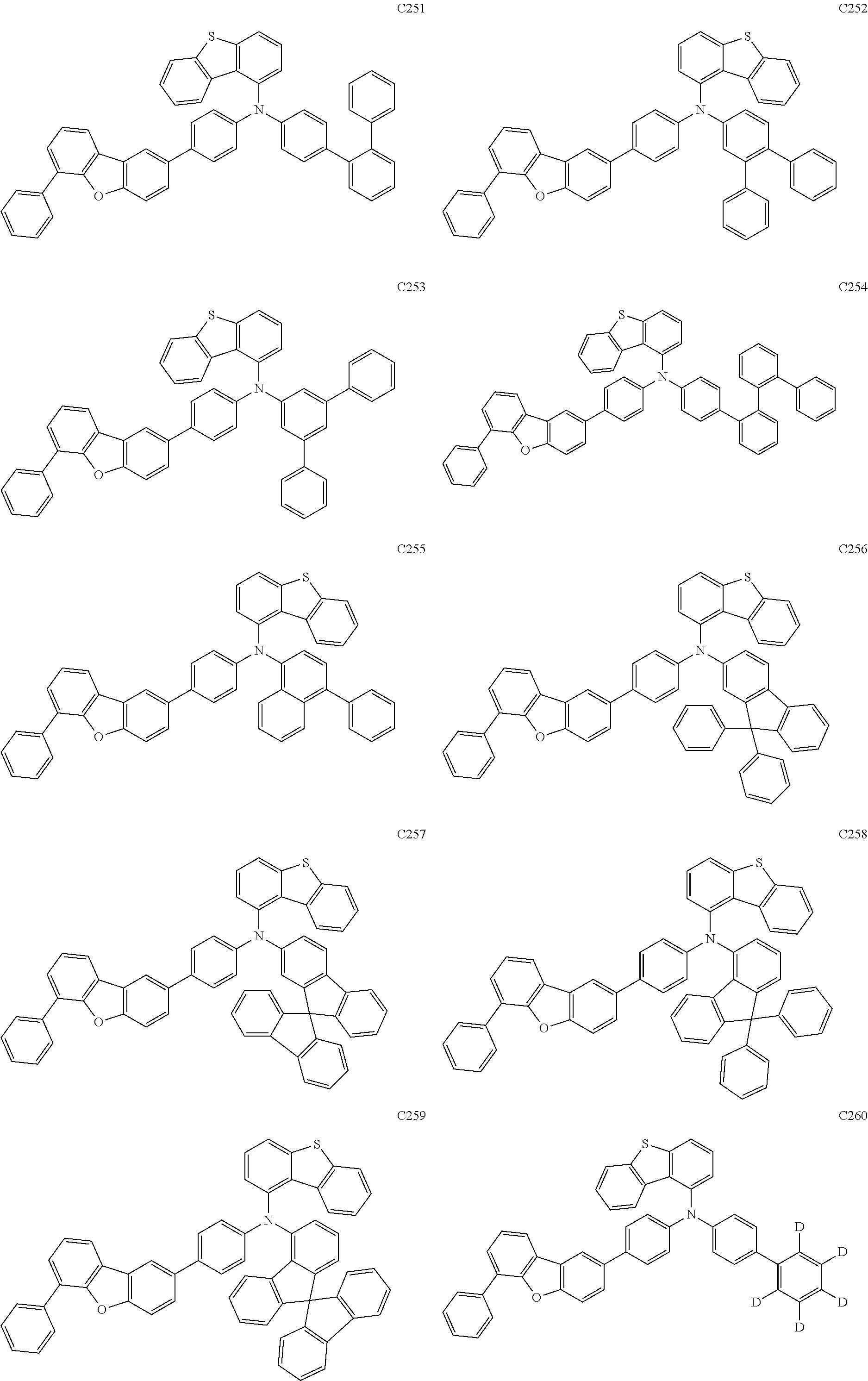

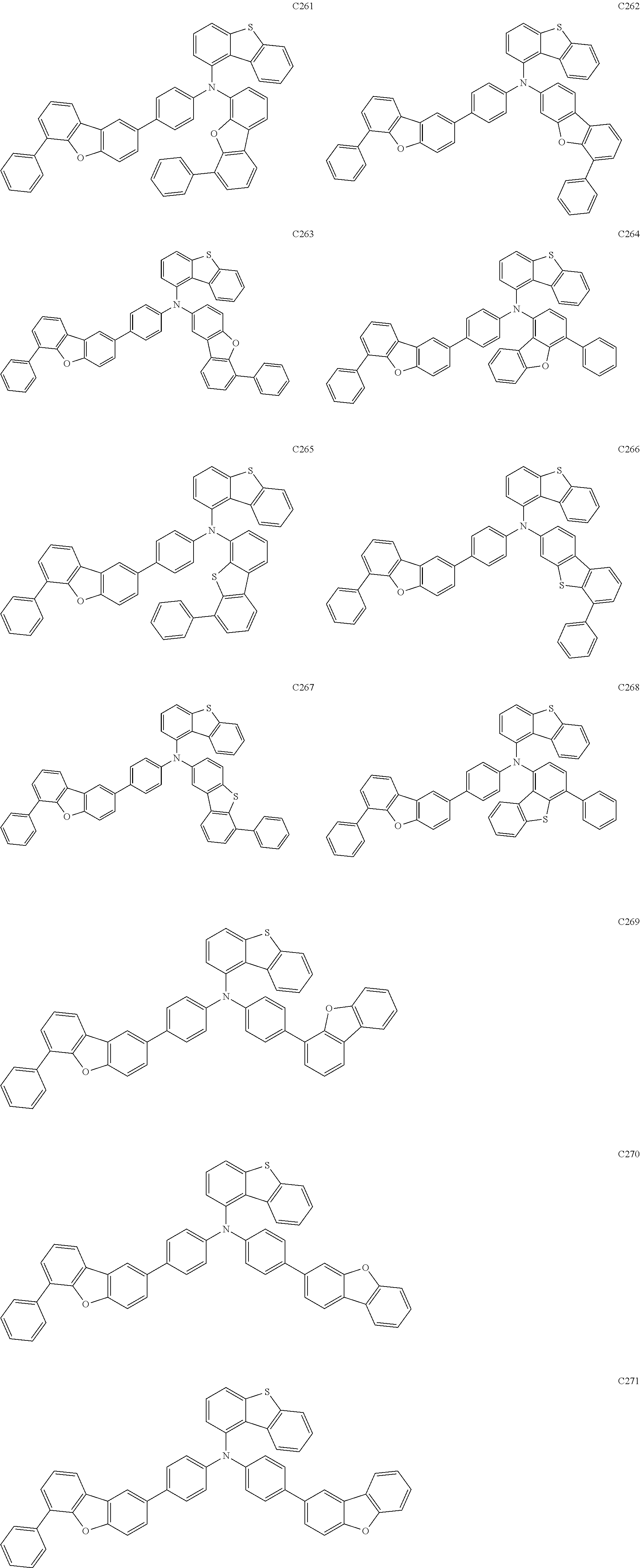

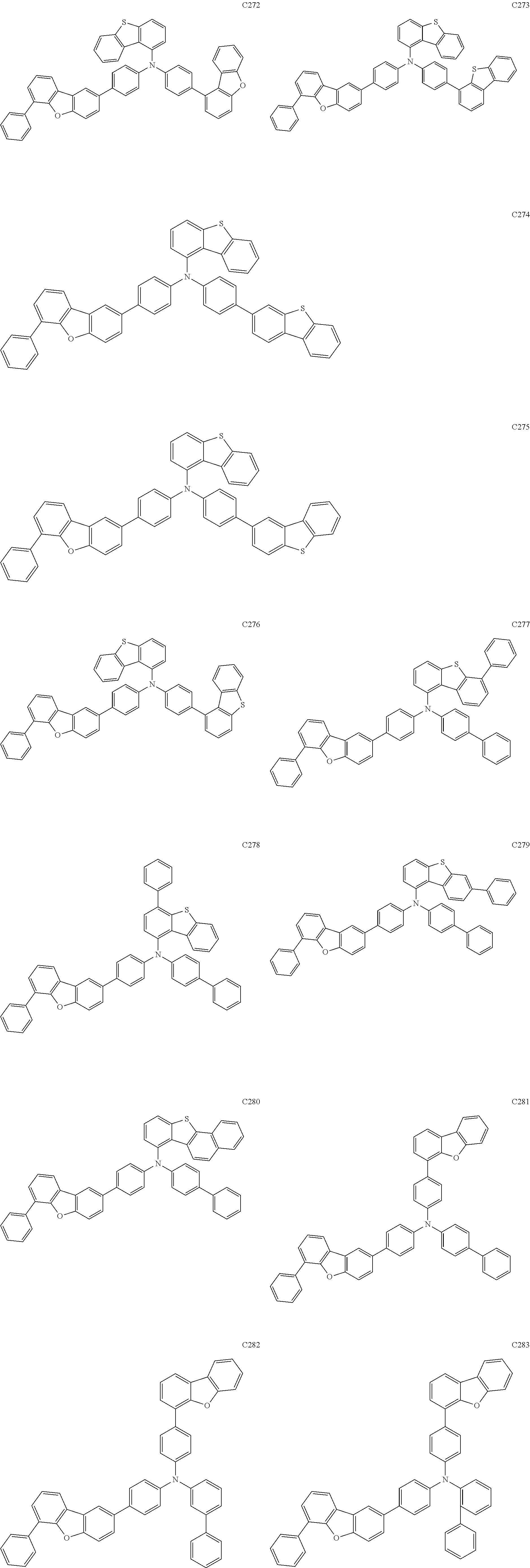

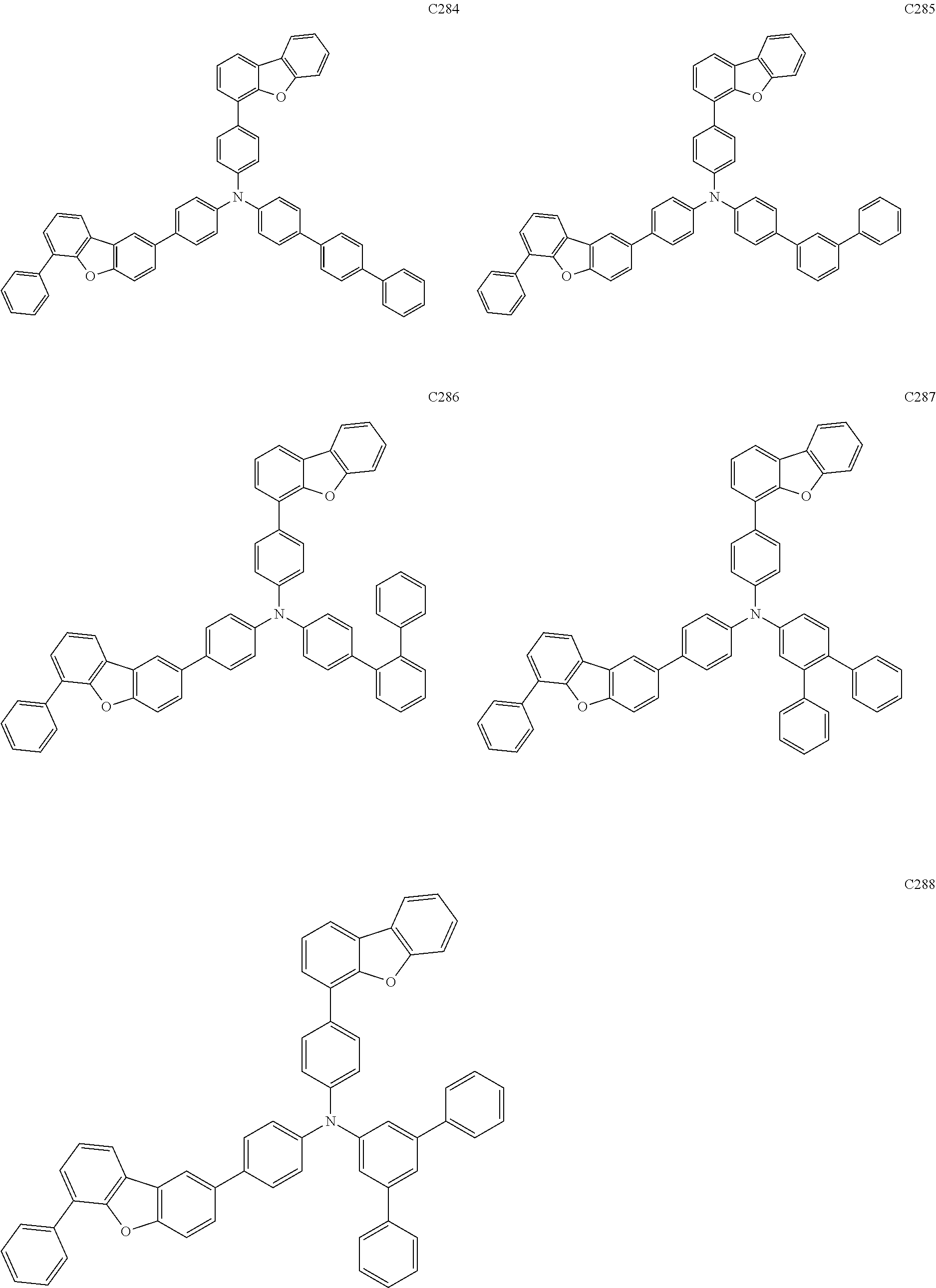

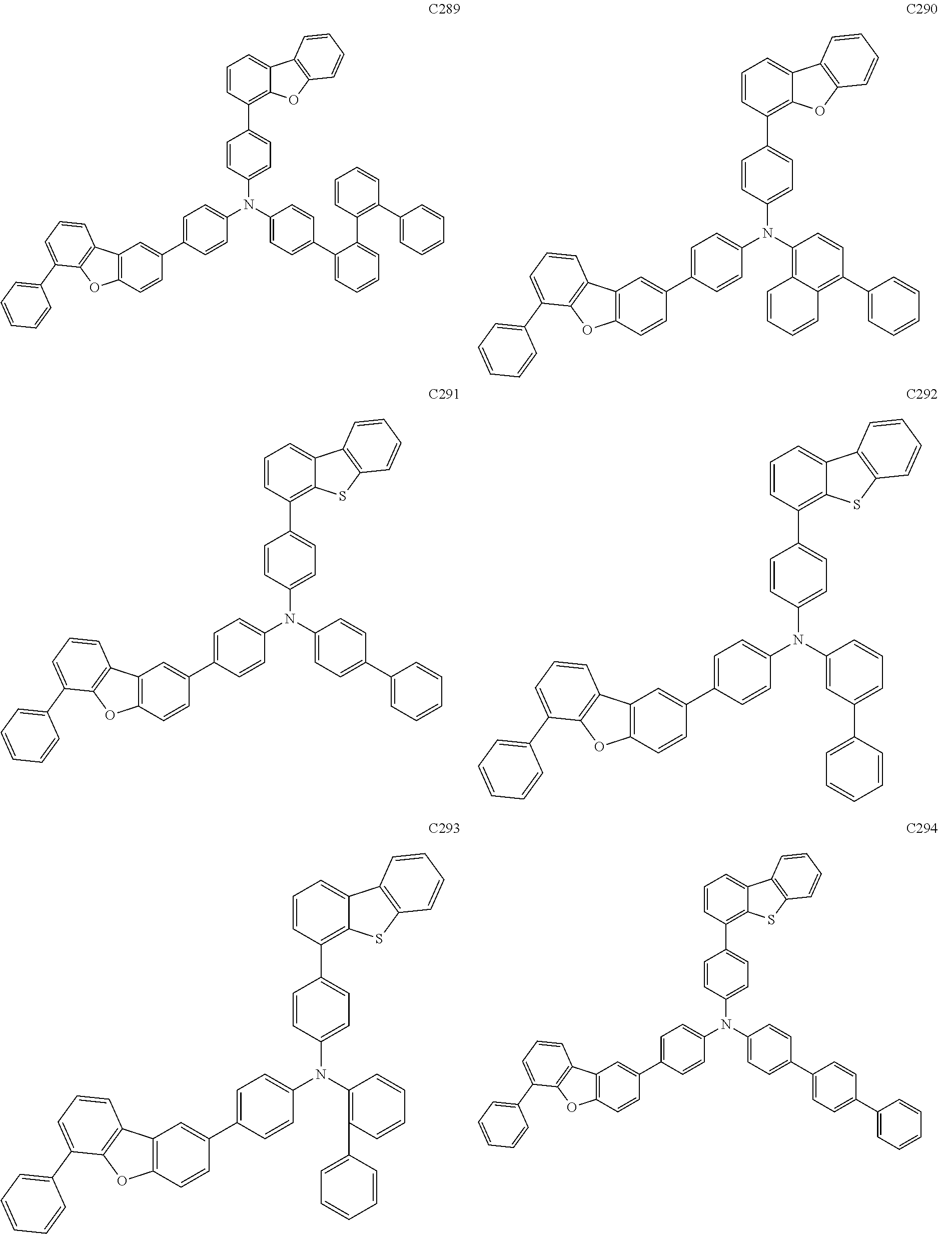

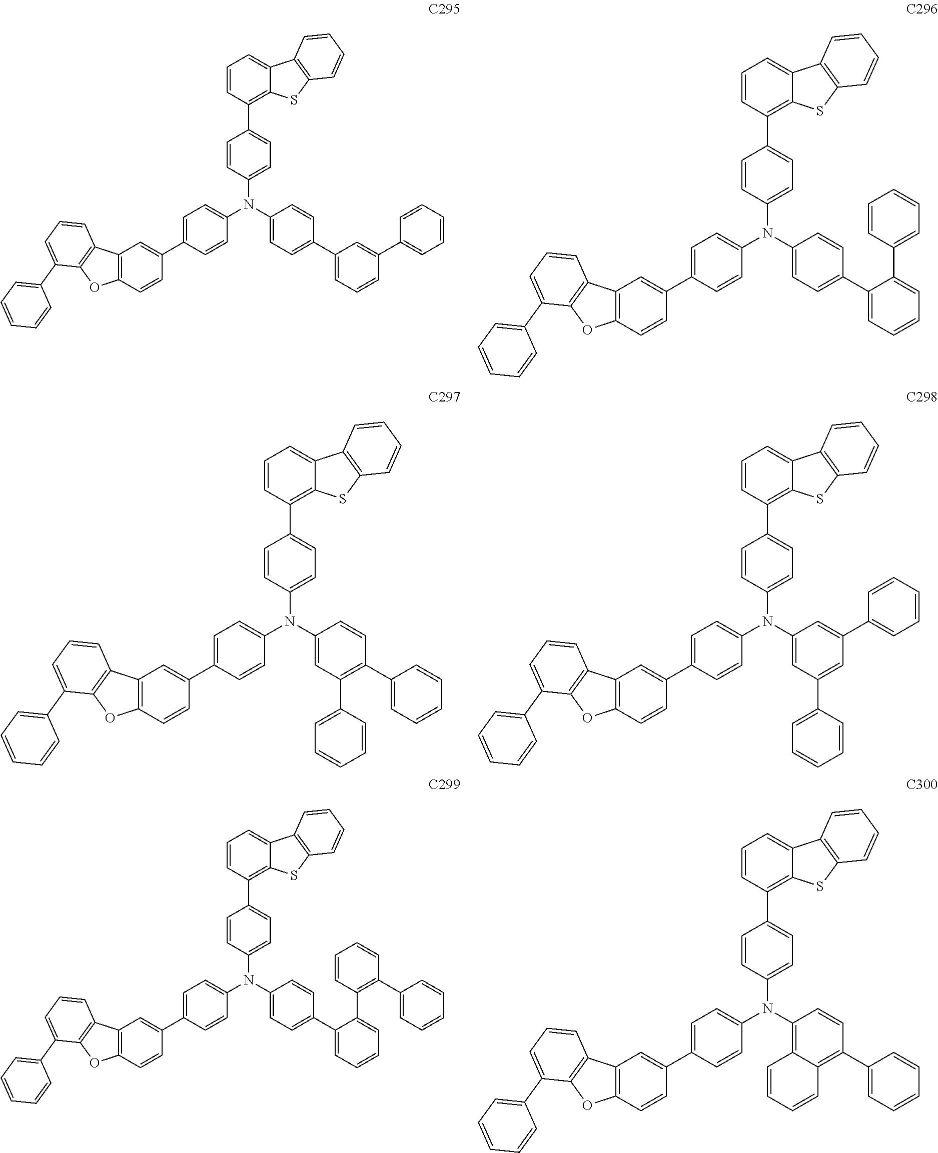

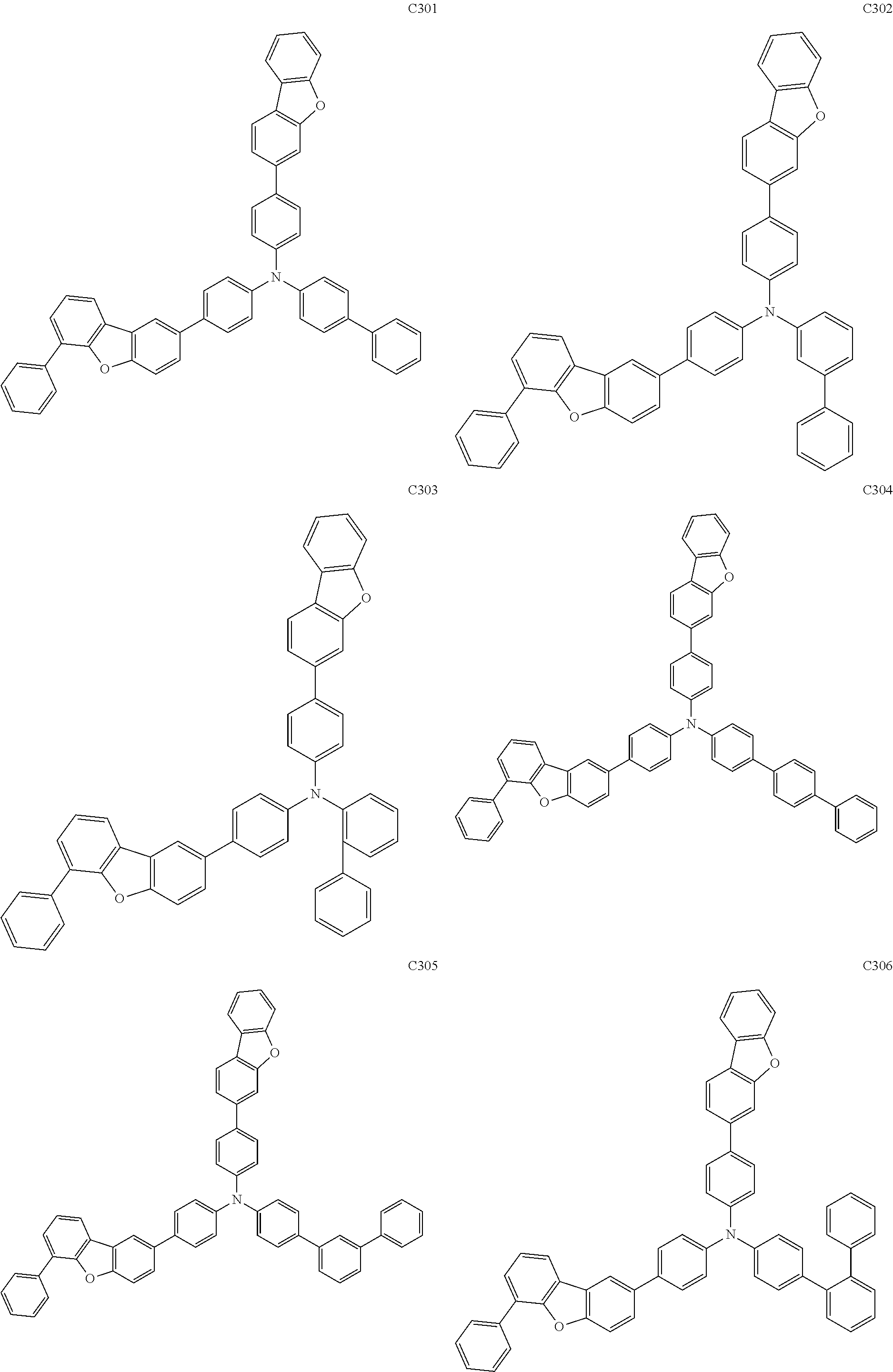

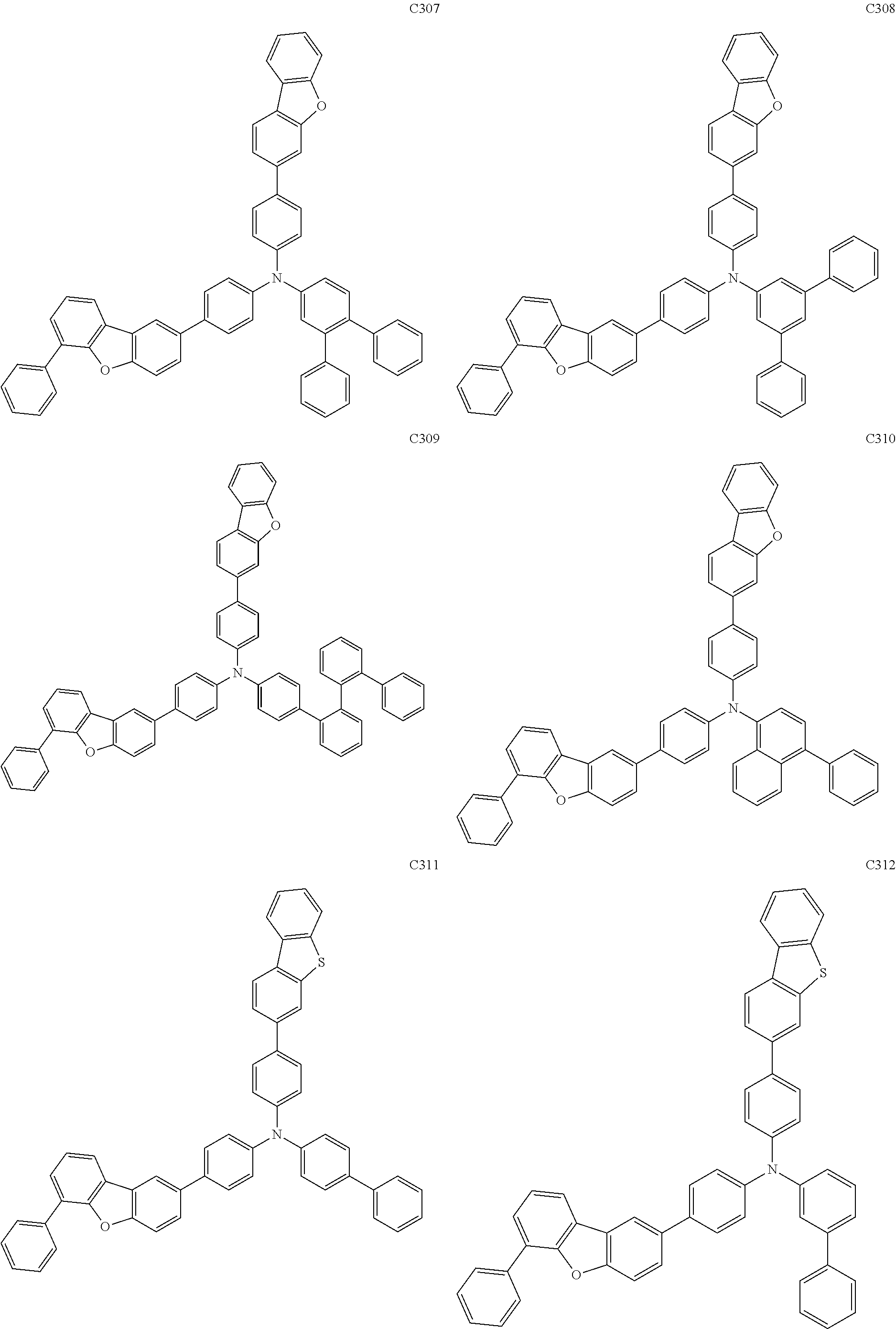

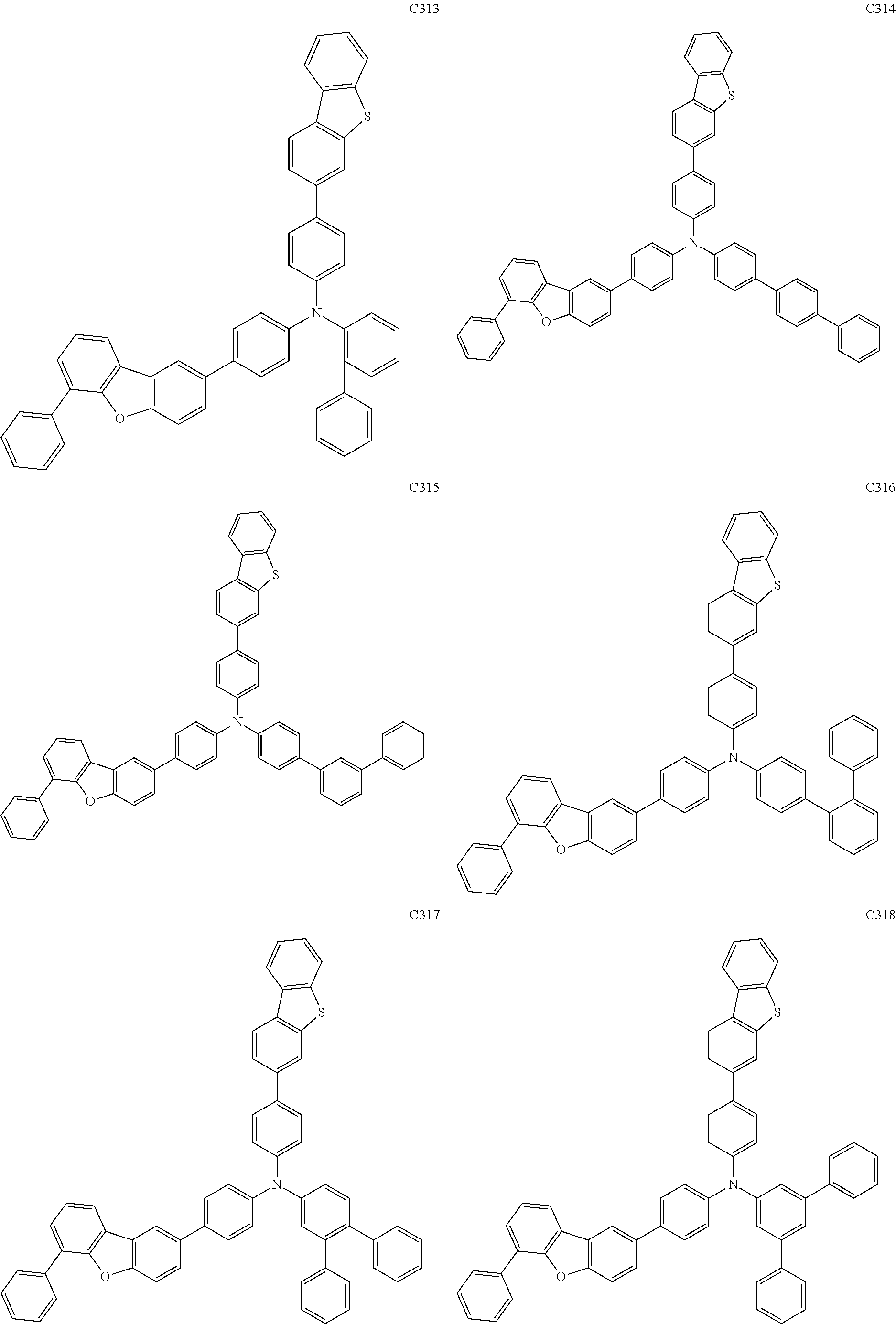

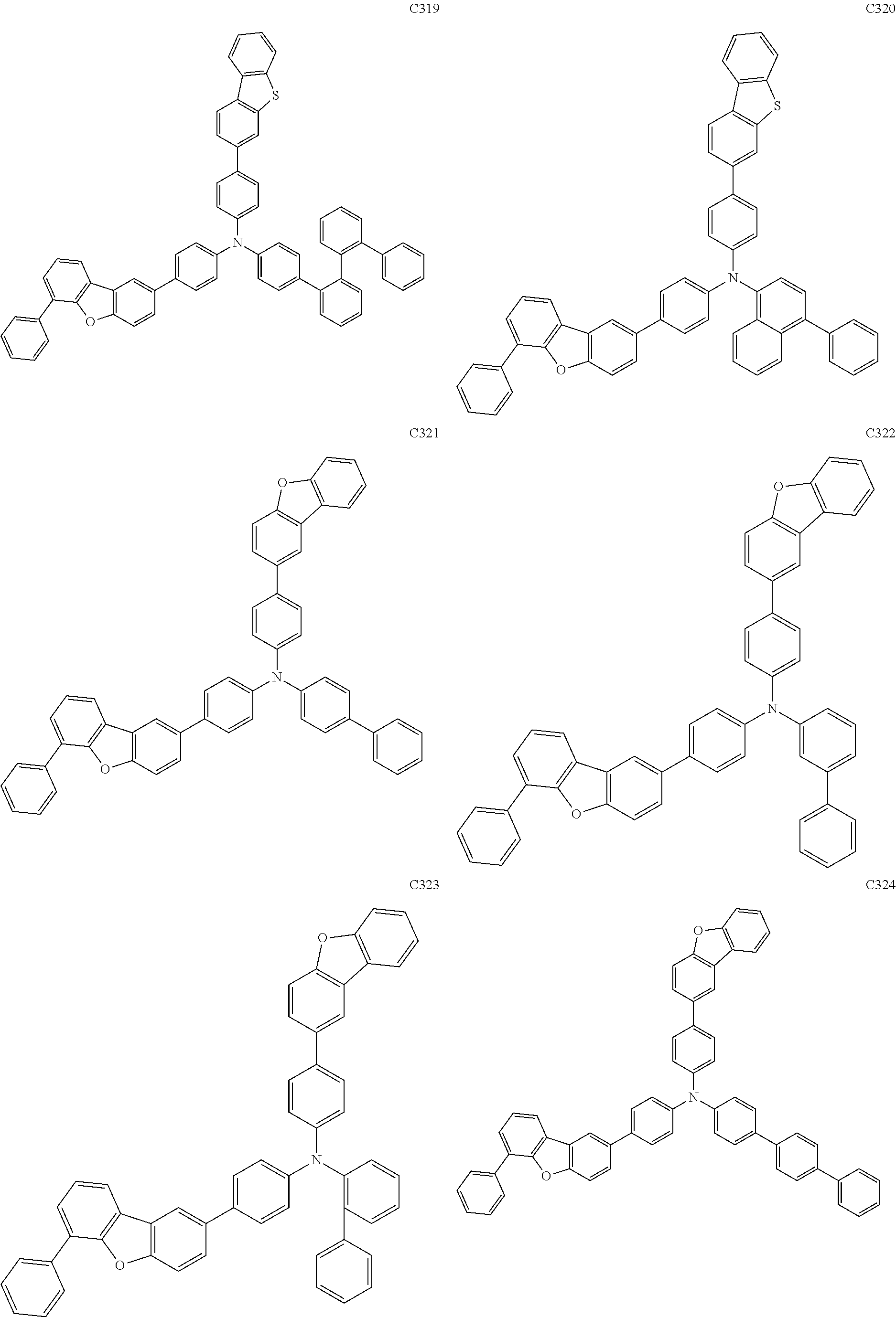

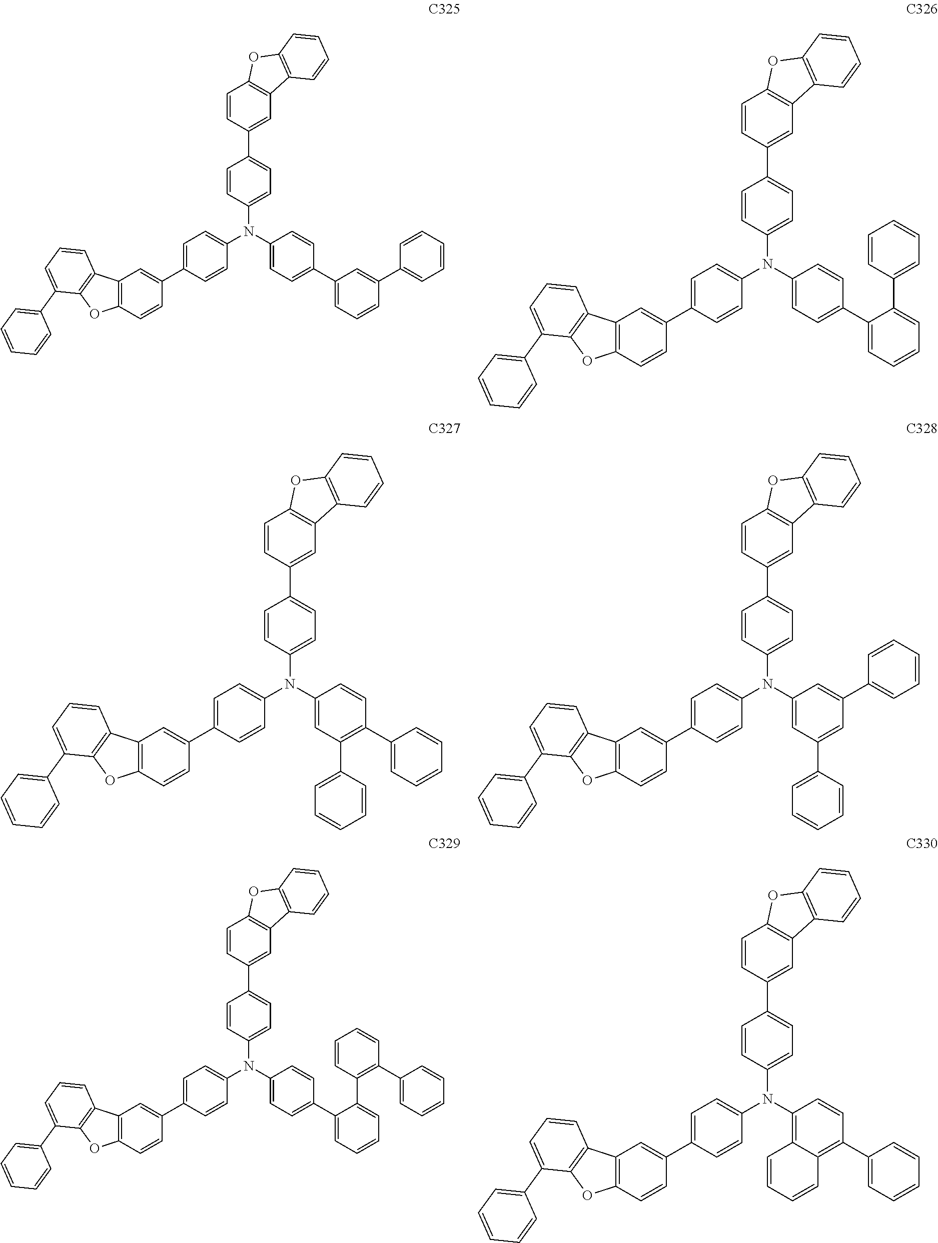

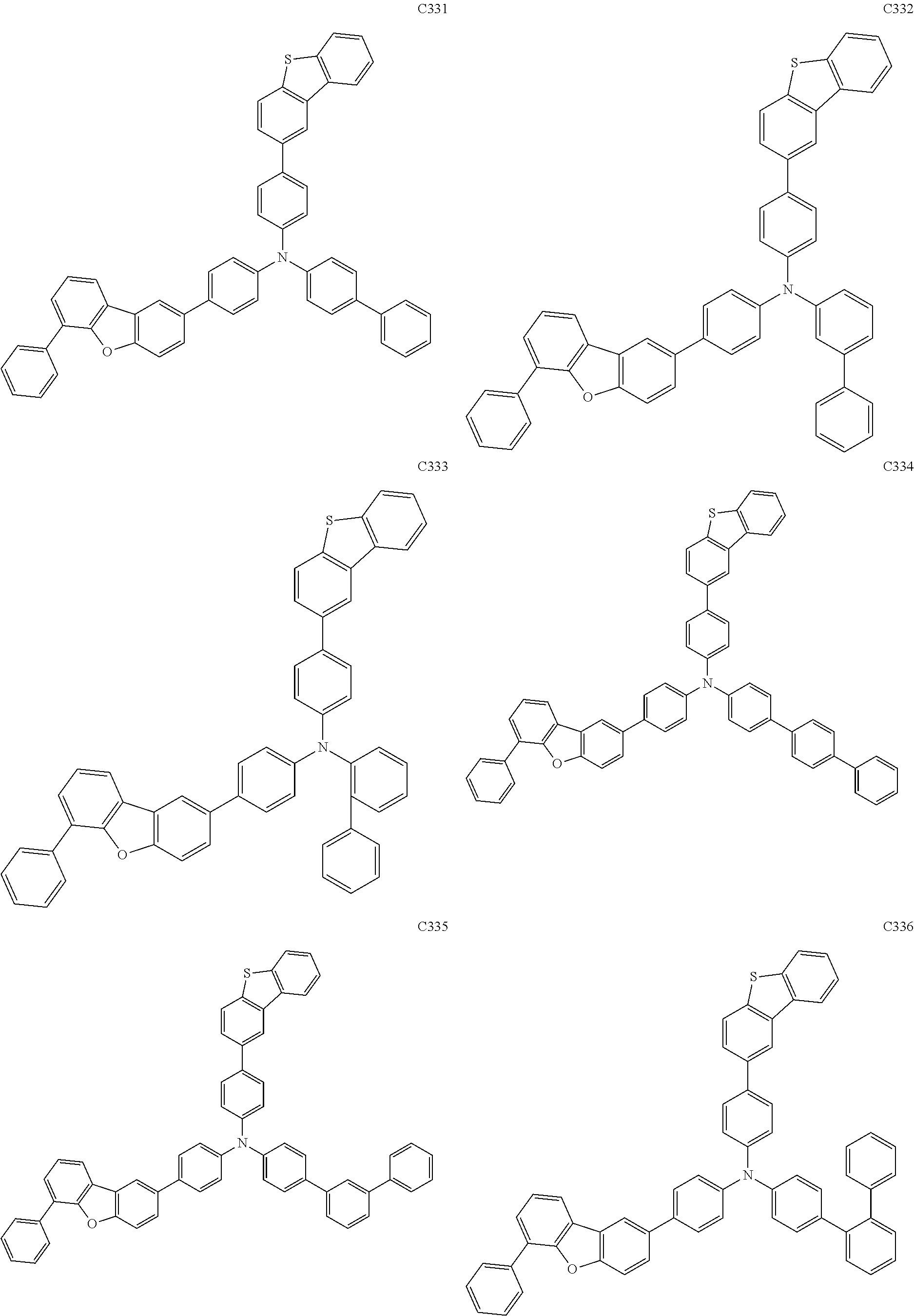

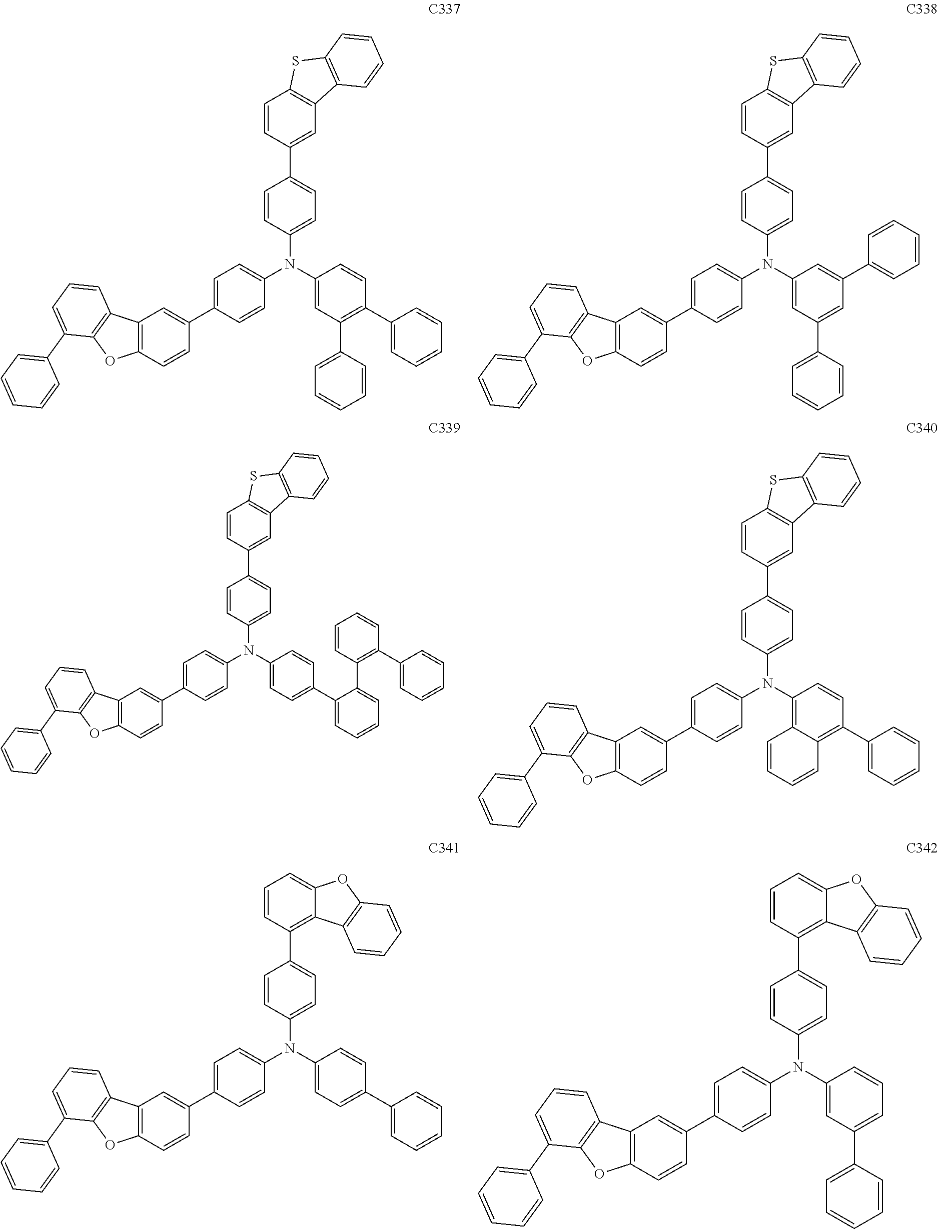

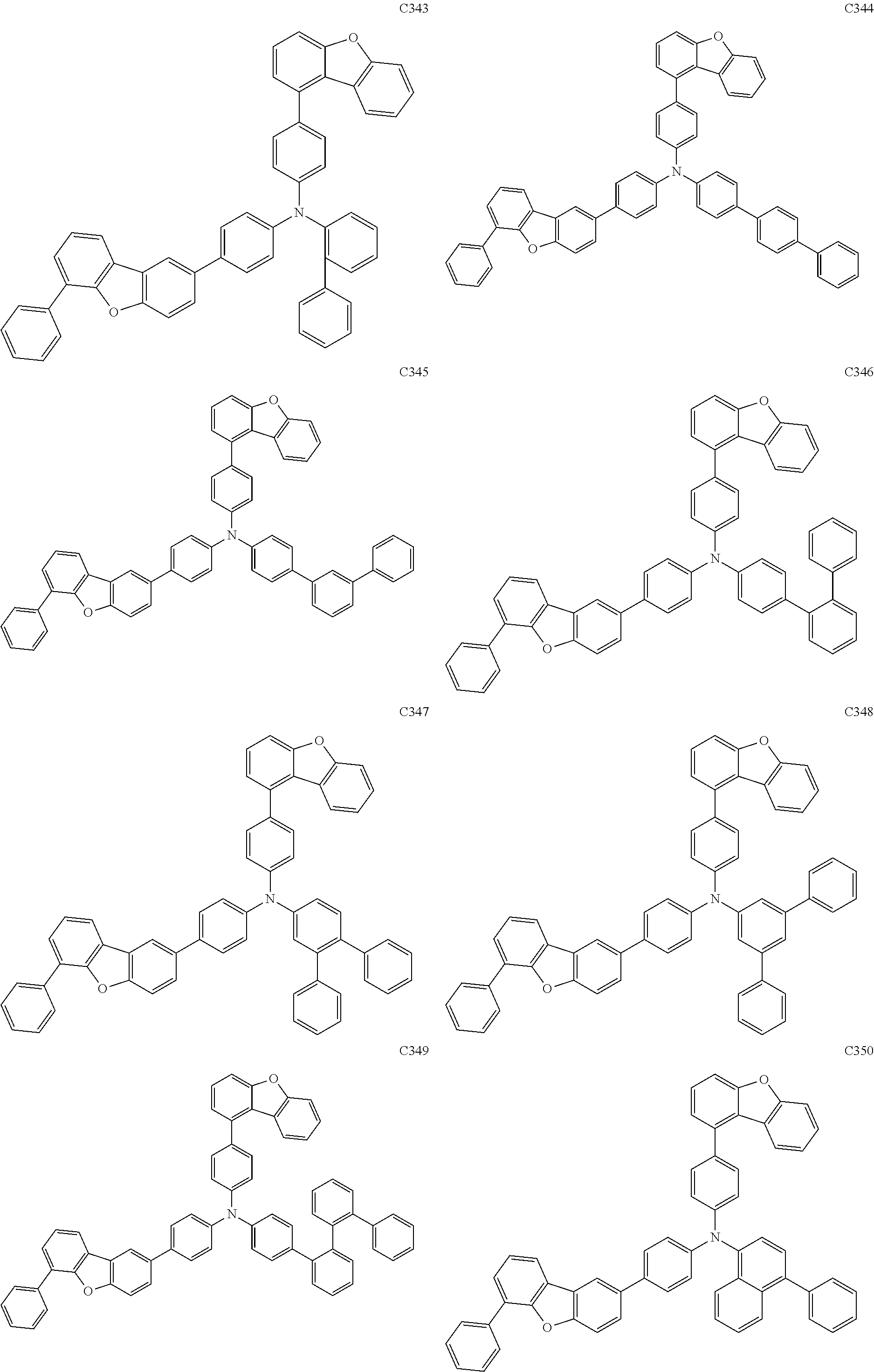

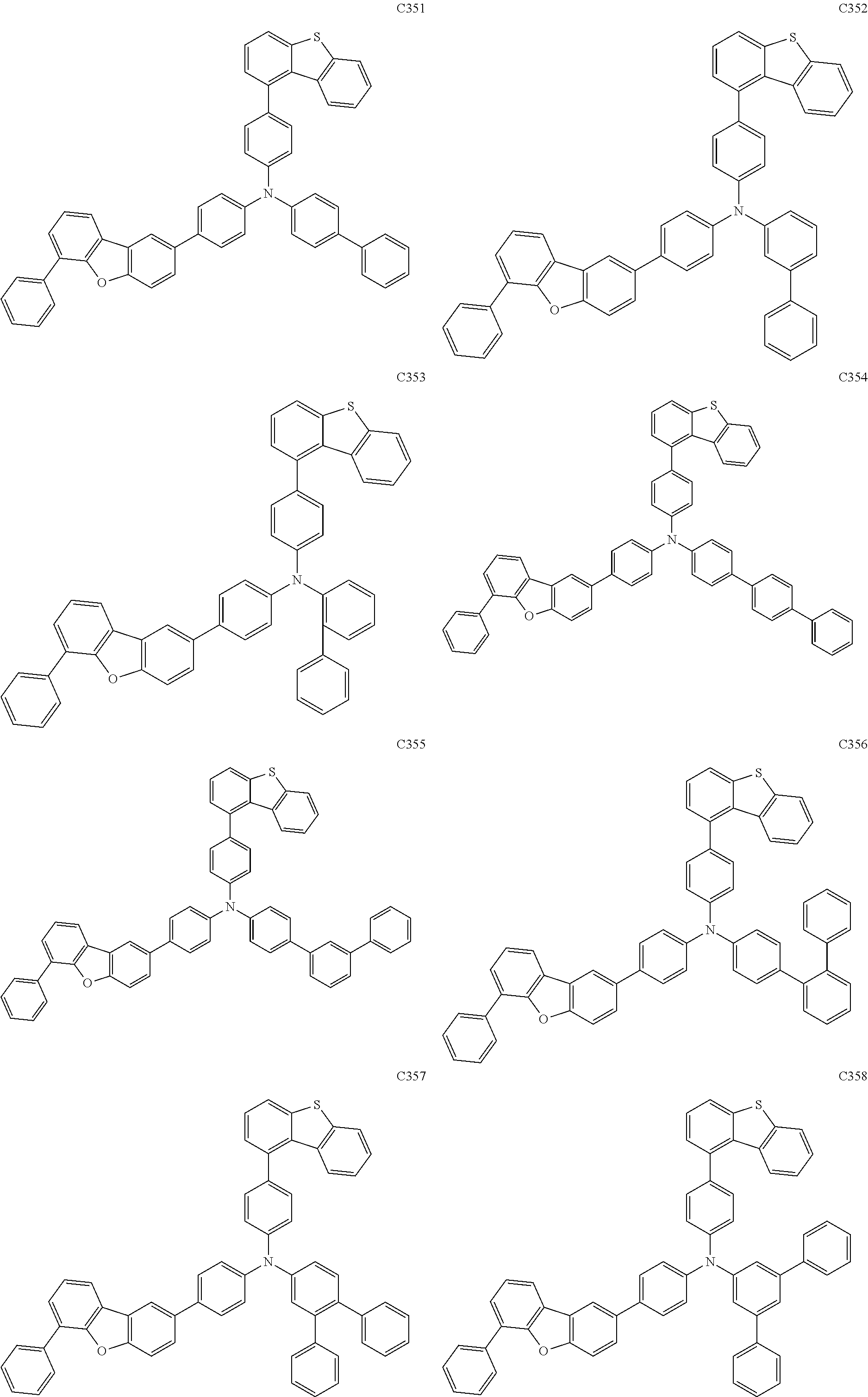

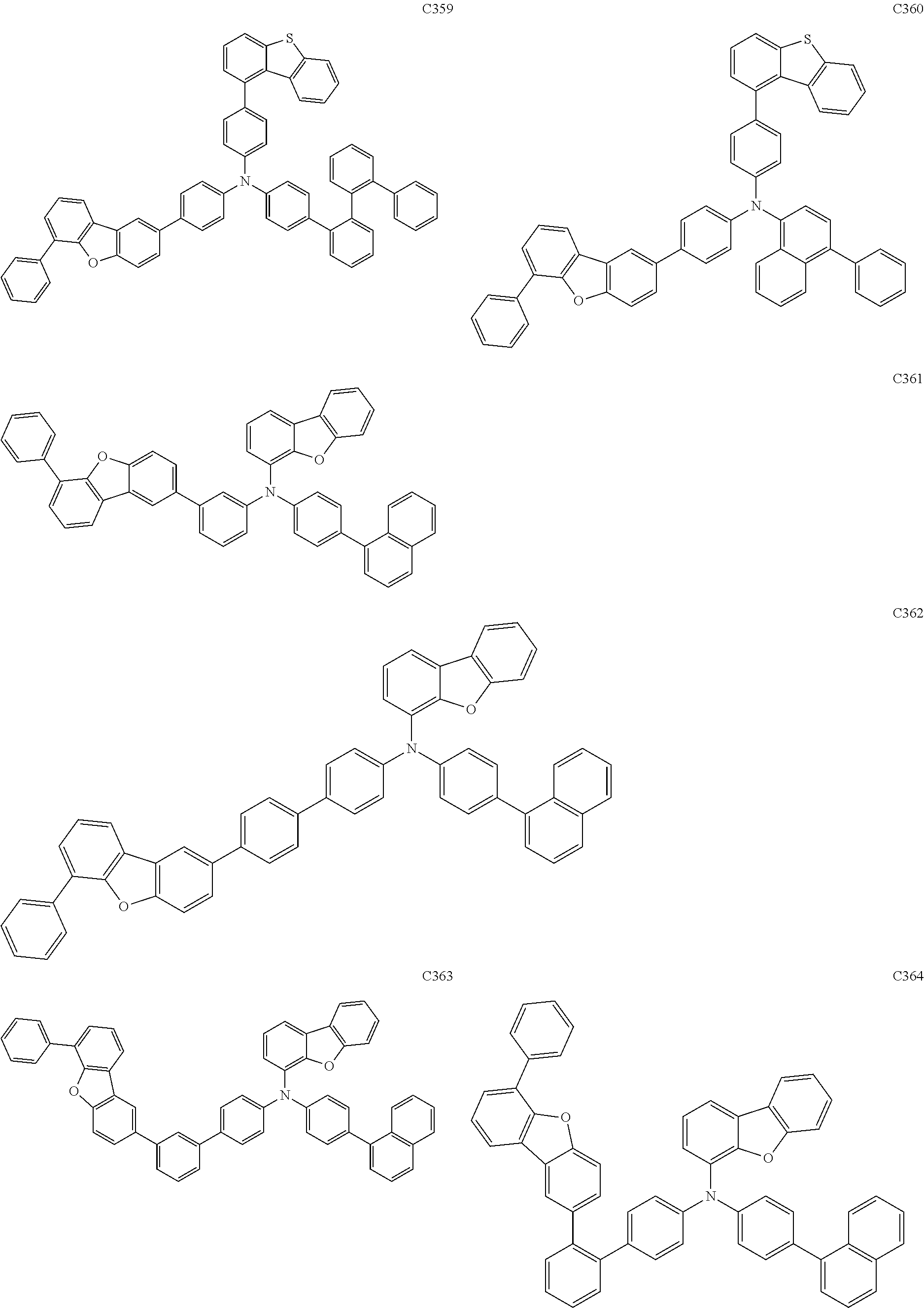

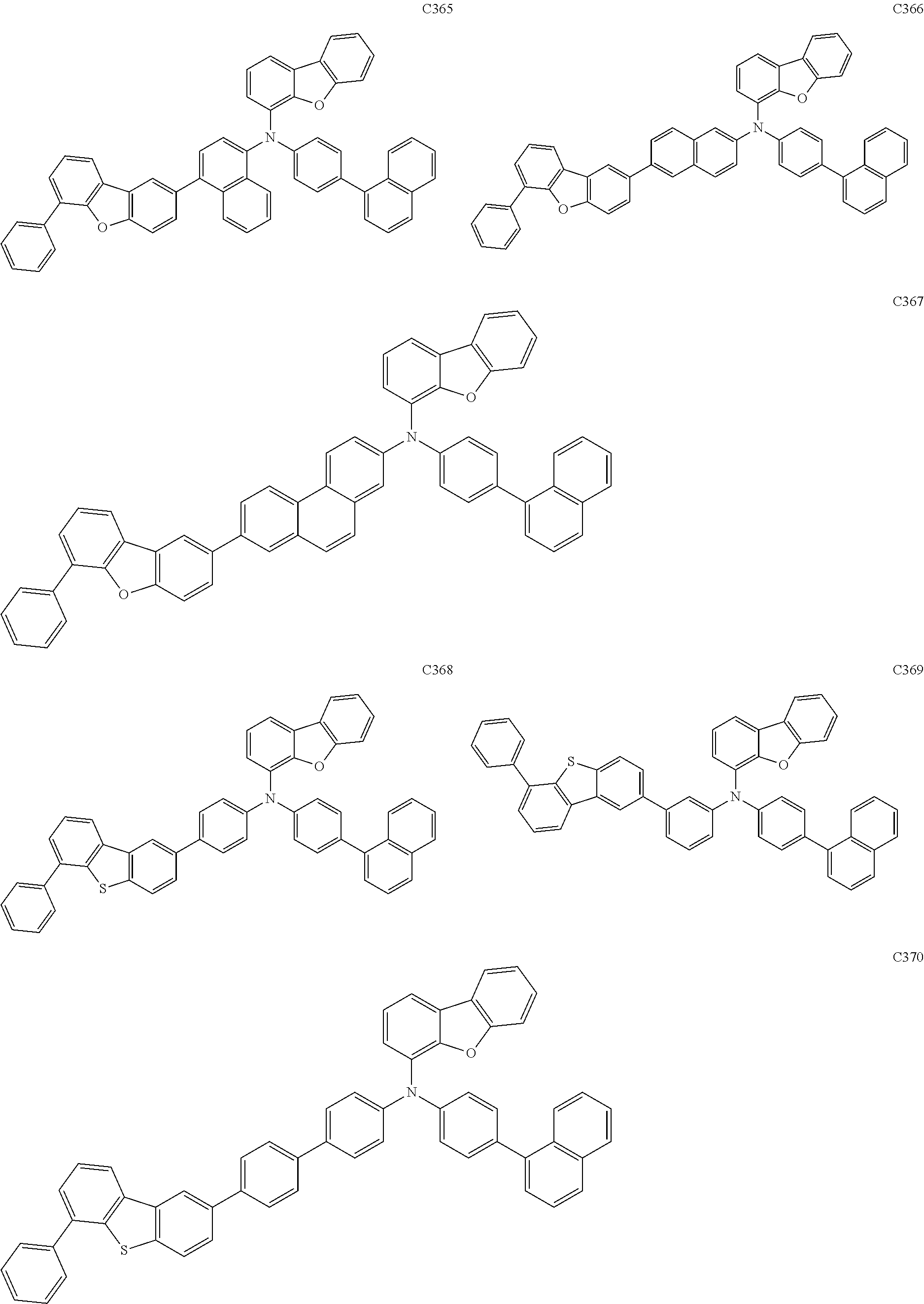

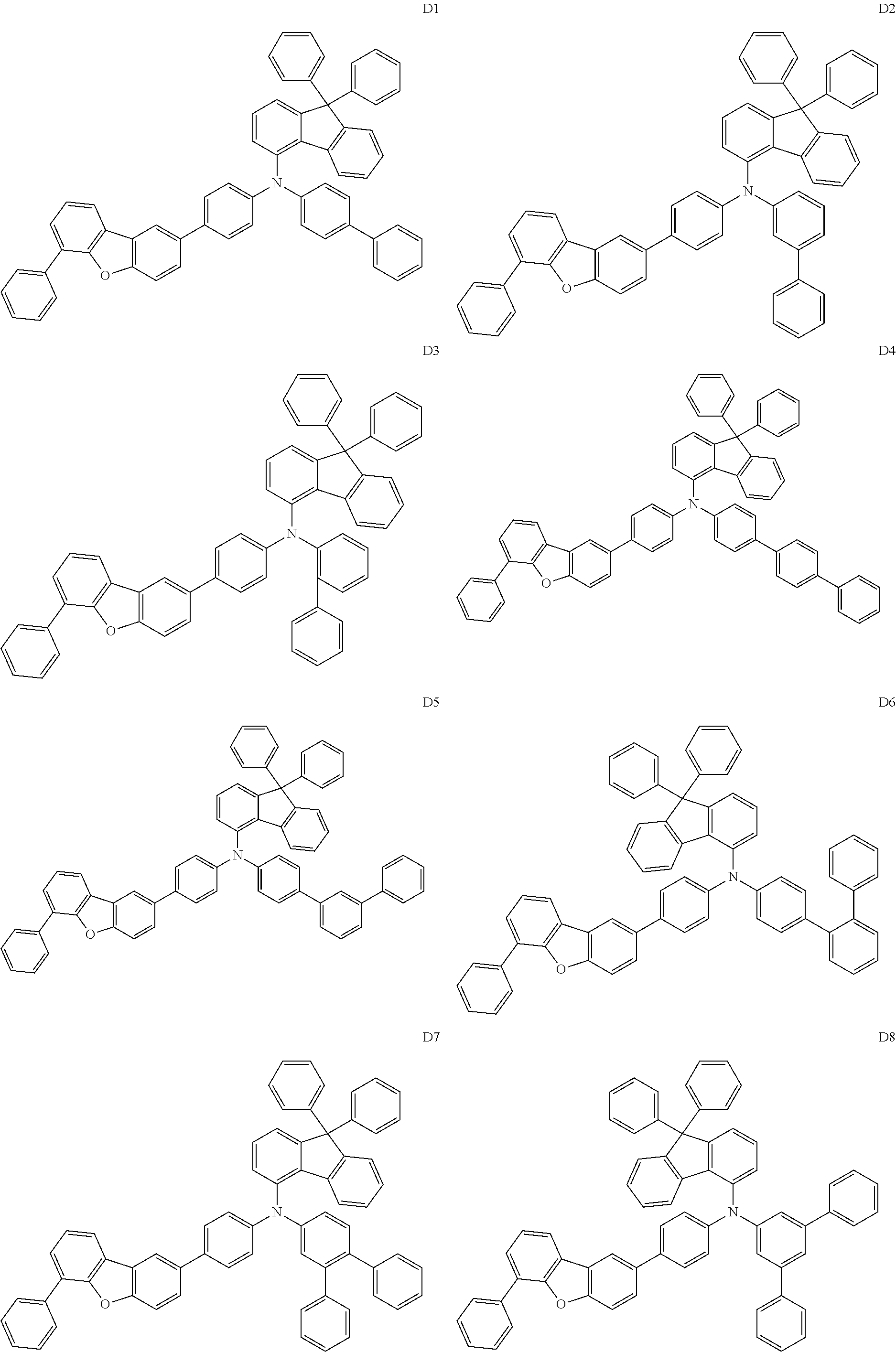

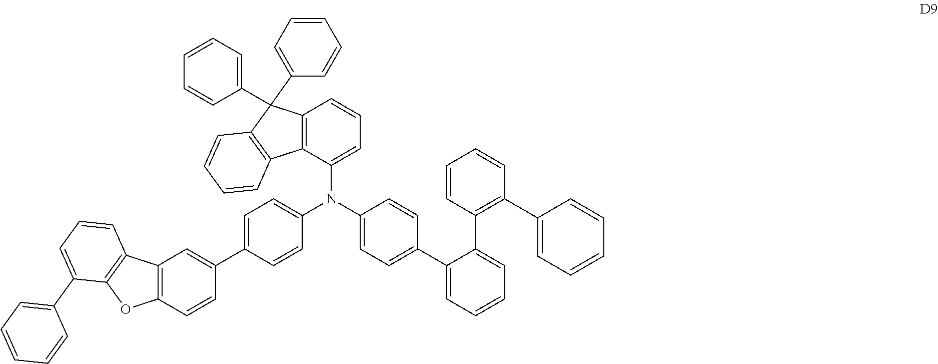 [SMILES: c1ccc(-c2ccccc2-c2ccccc2-c2ccc(N(c3ccc(-c4ccc5oc6c(-c7ccccc7)cccc6c5c4)cc3)c3cccc4c3-c3ccccc3C4(c3ccccc3)c3ccccc3)cc2)cc1]

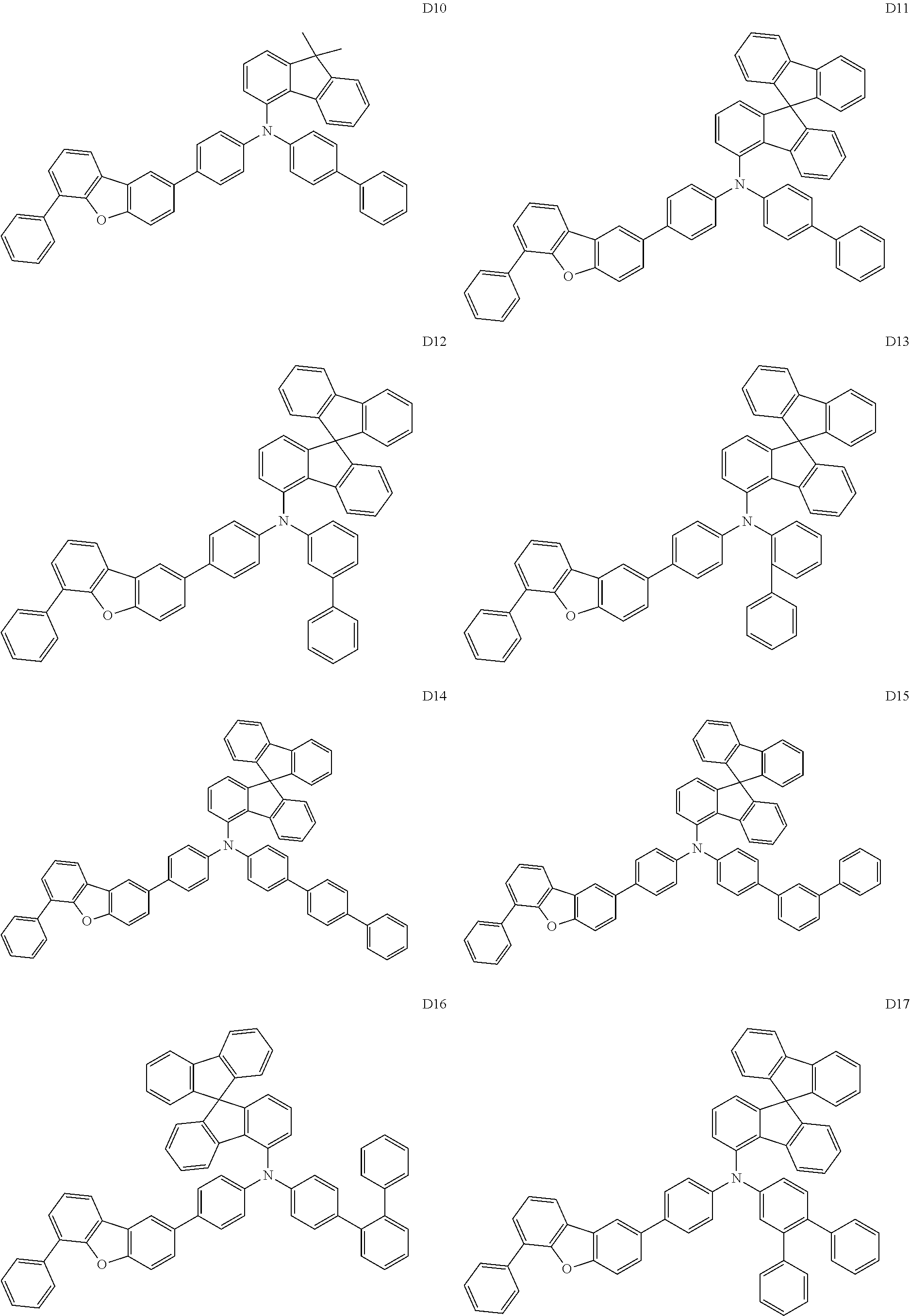

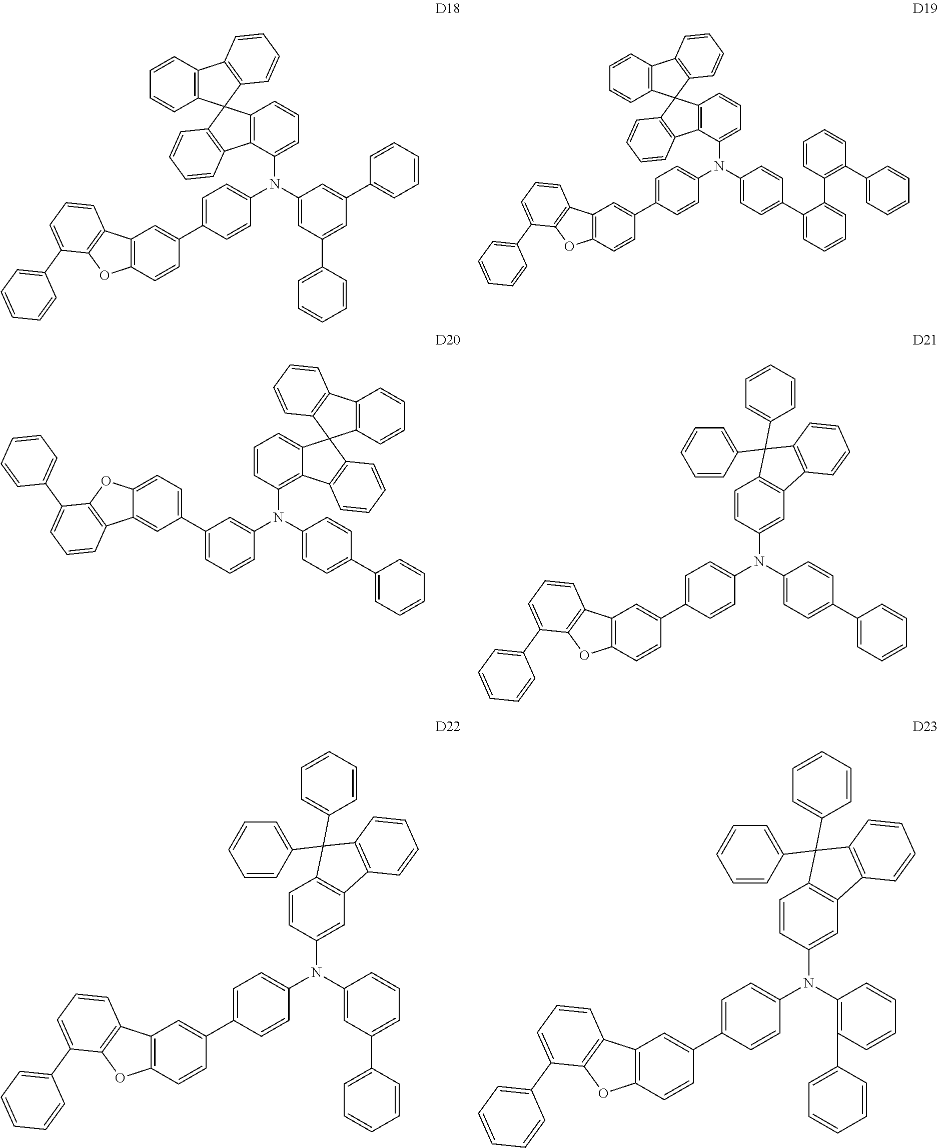

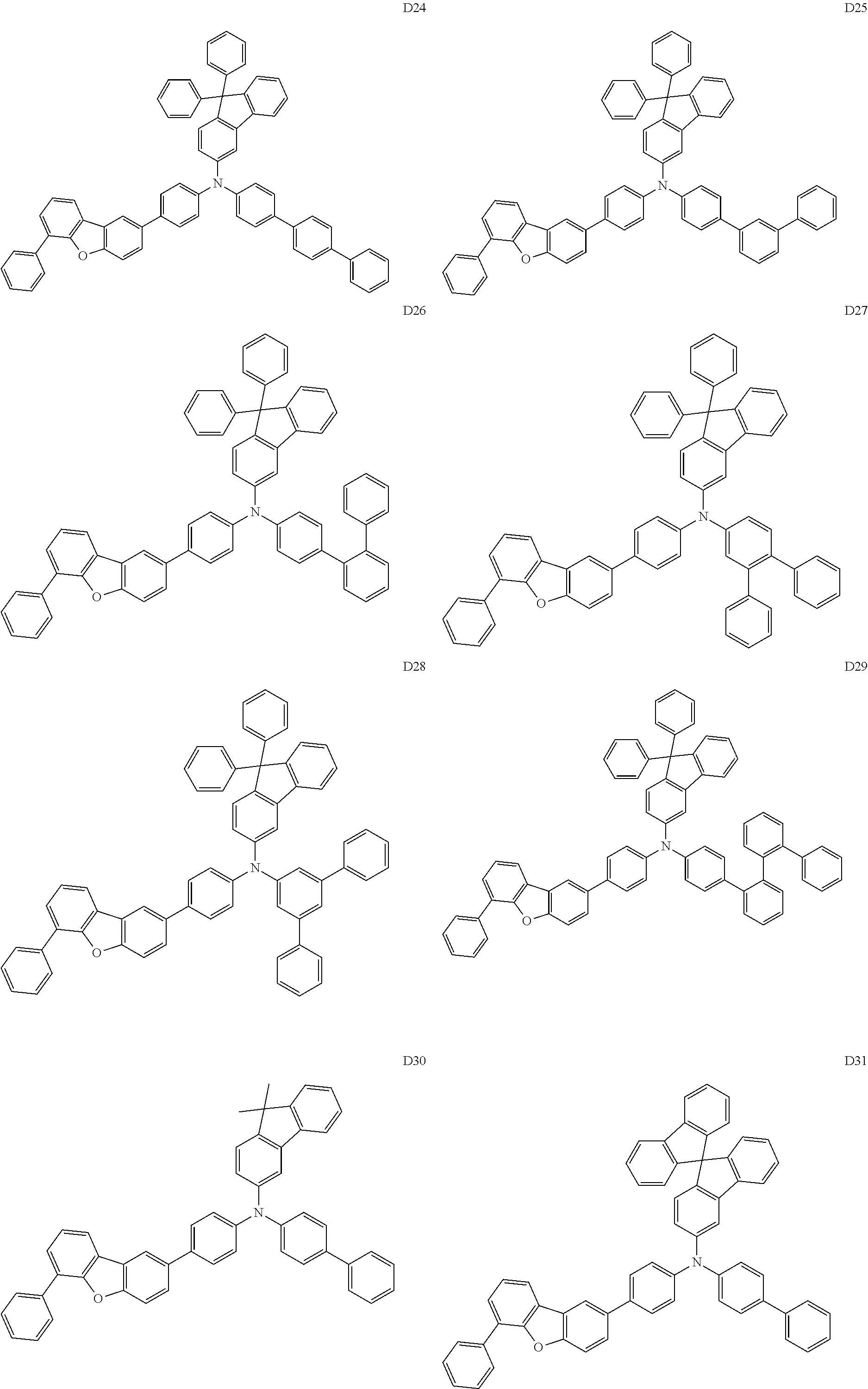

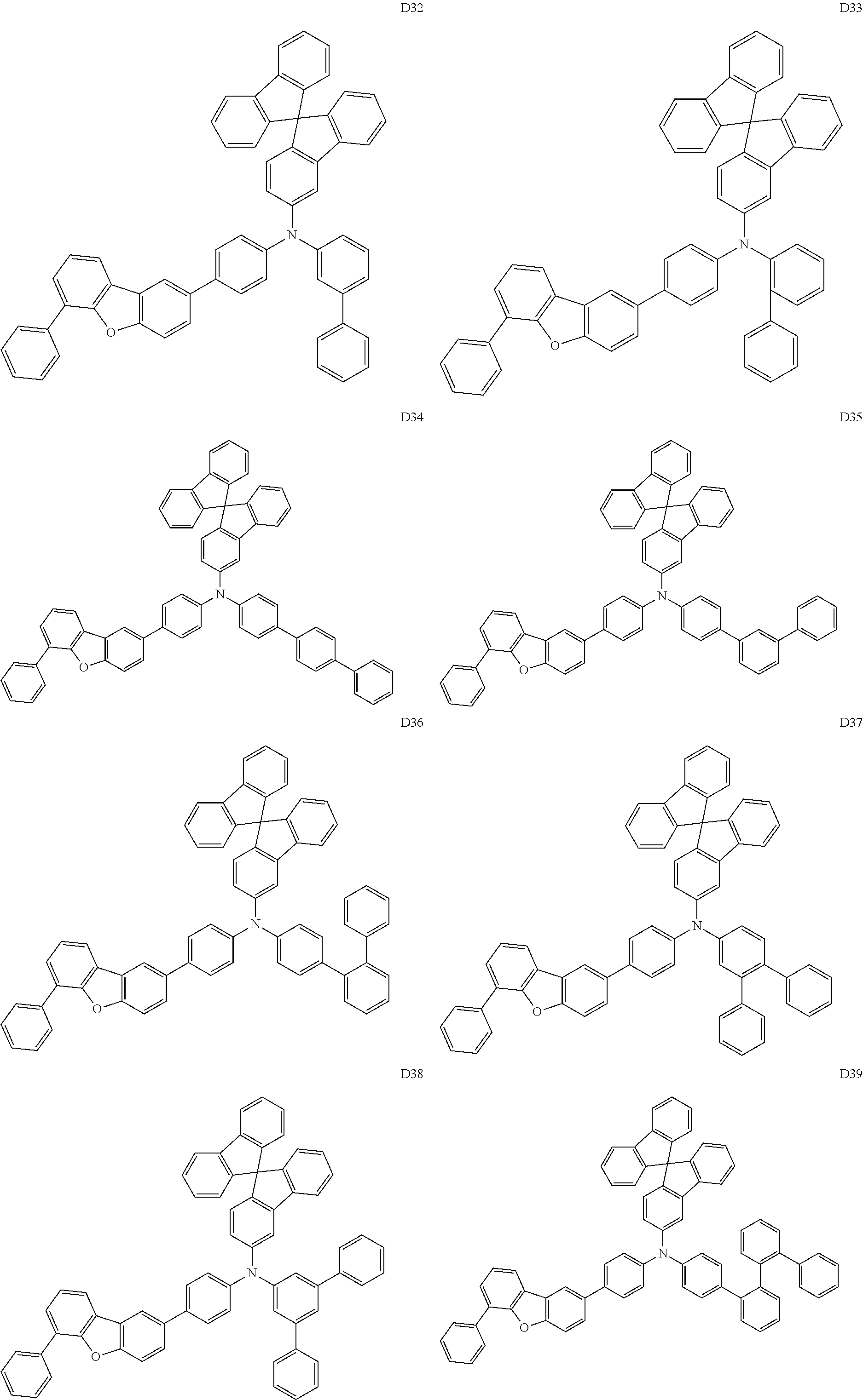

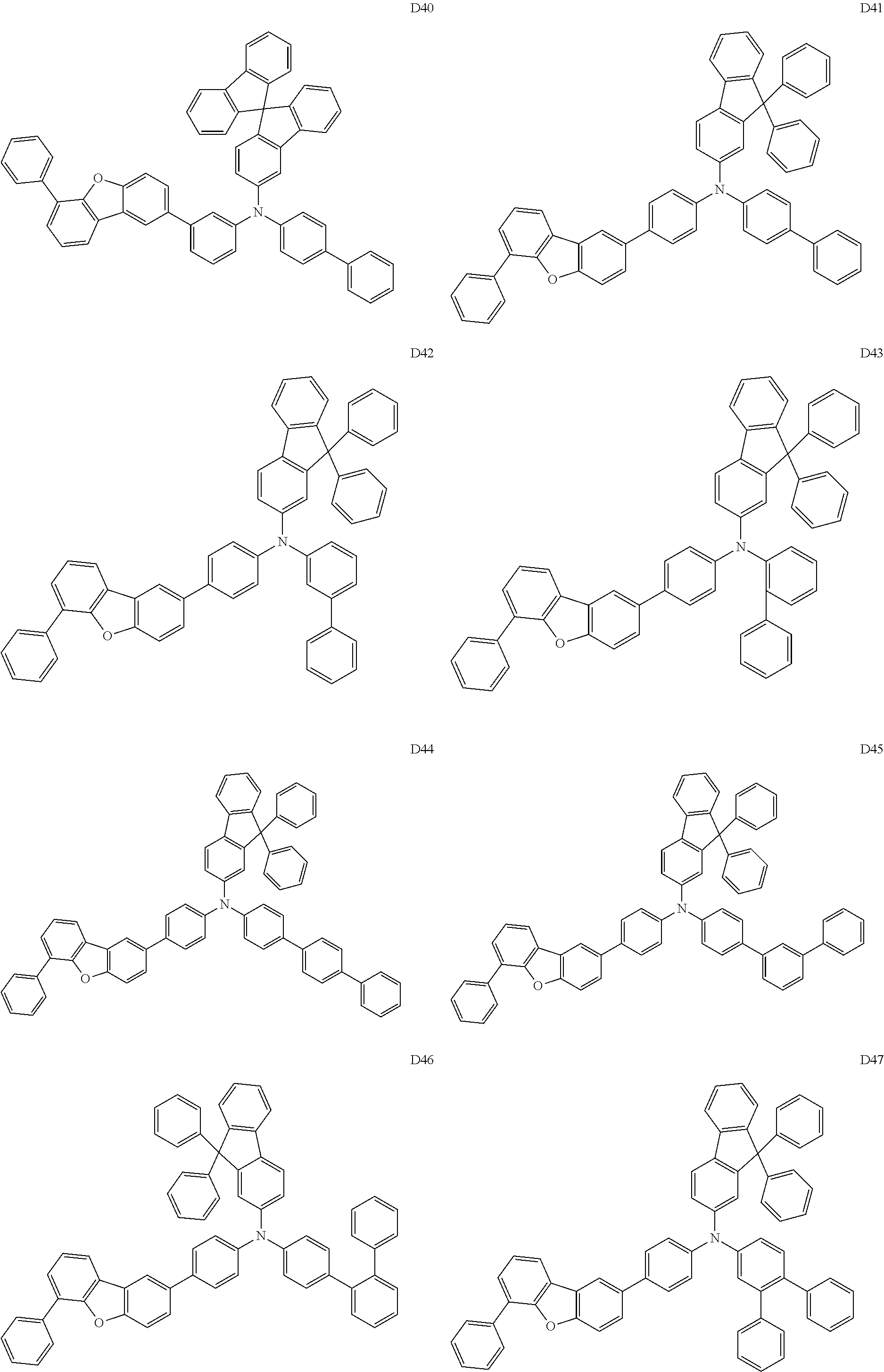

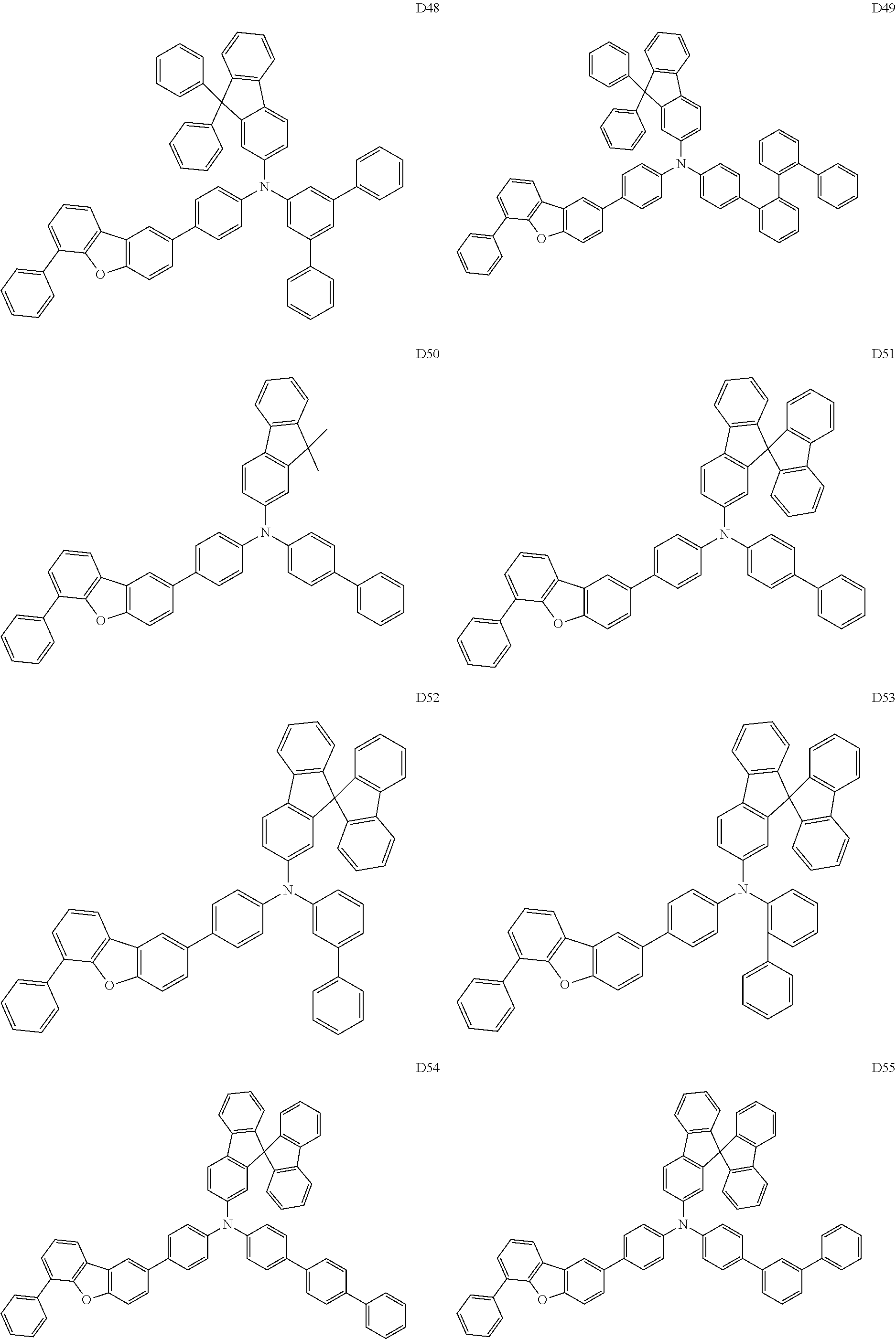

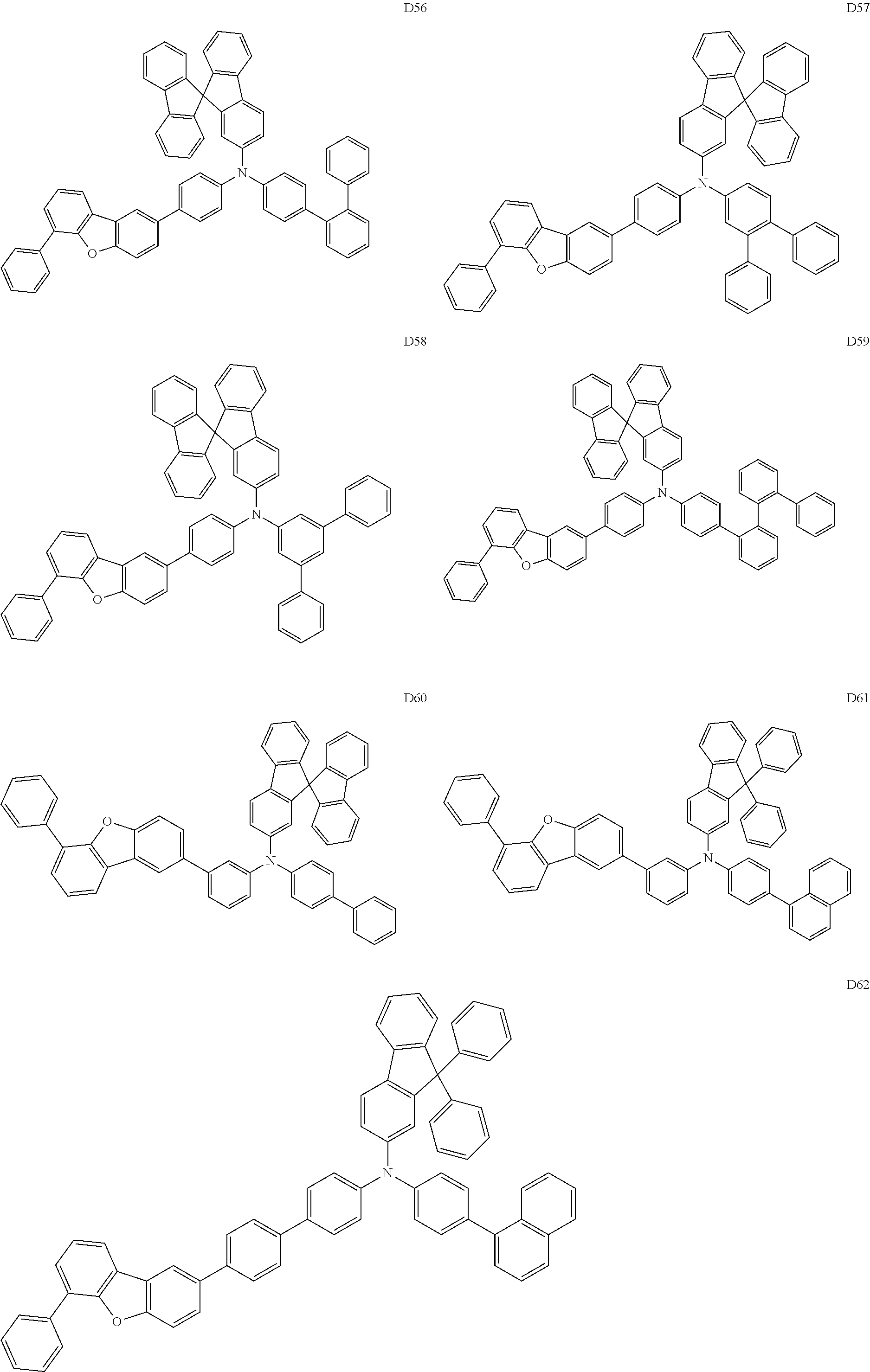

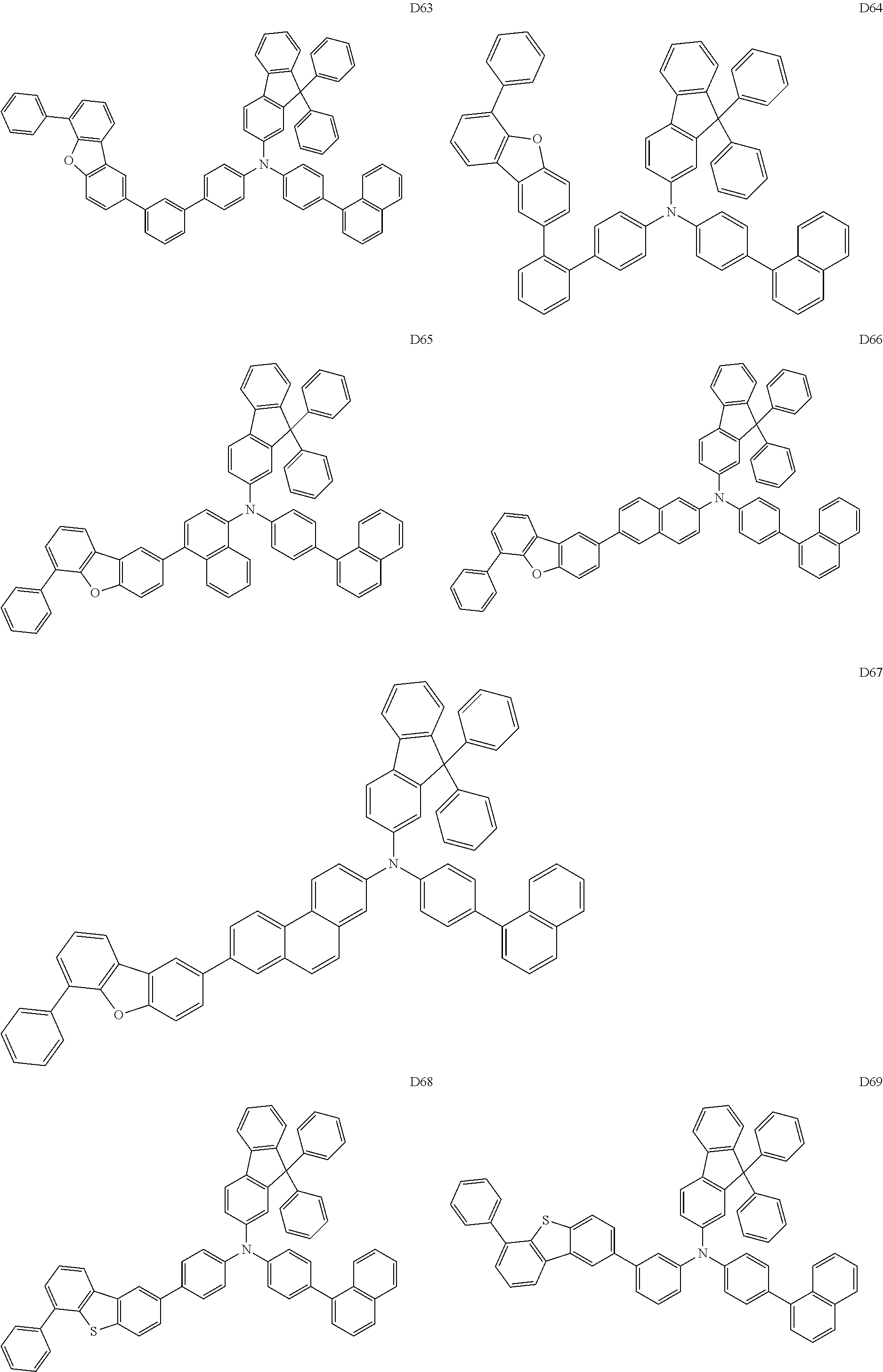

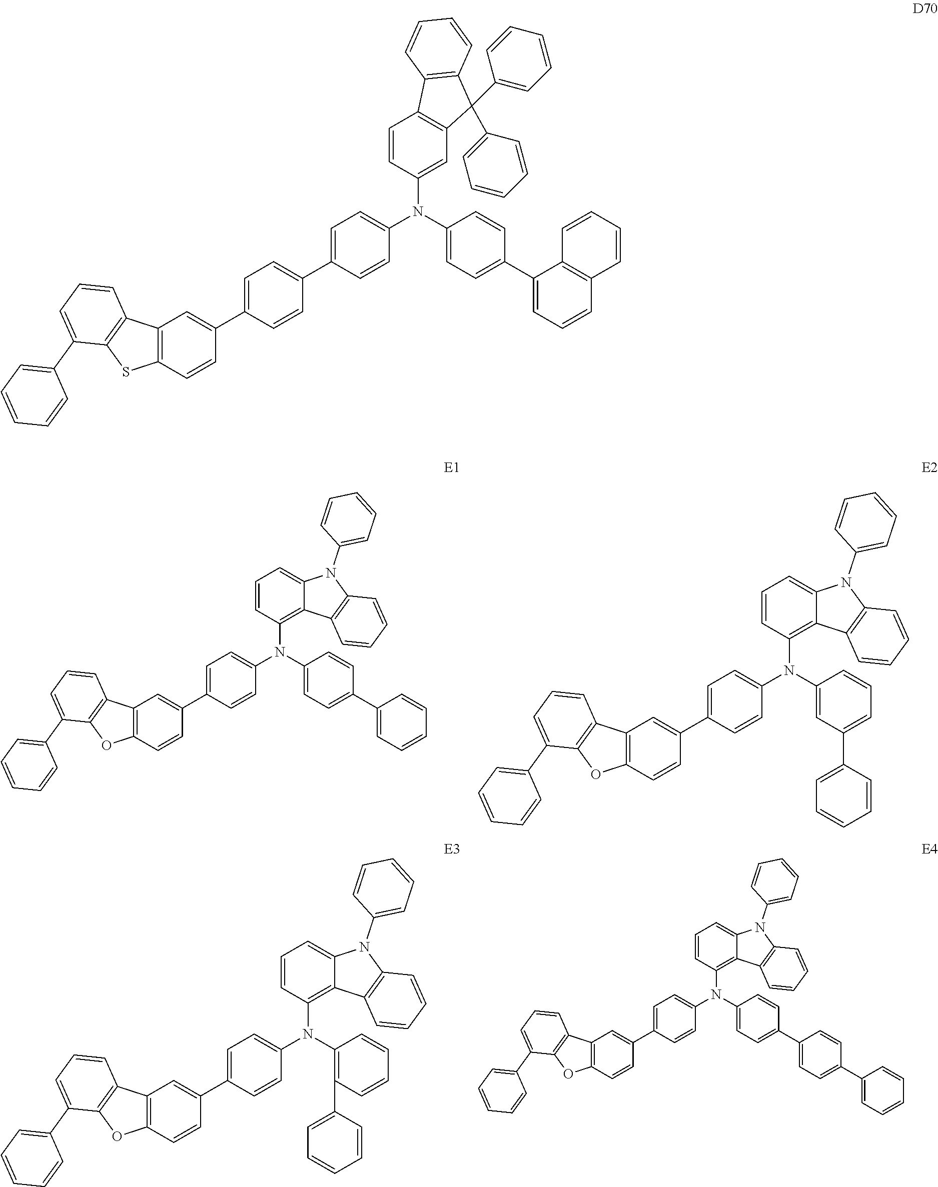

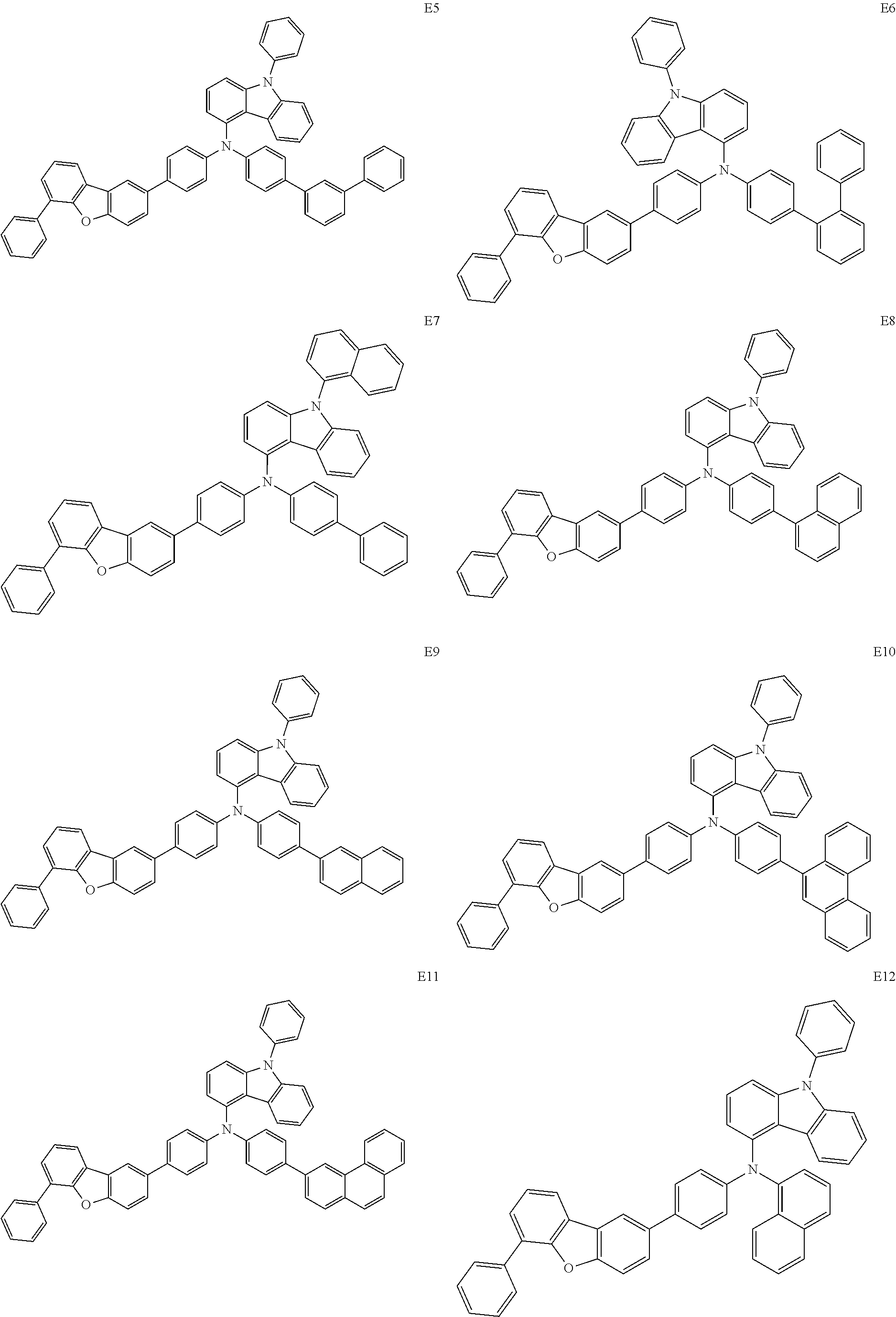

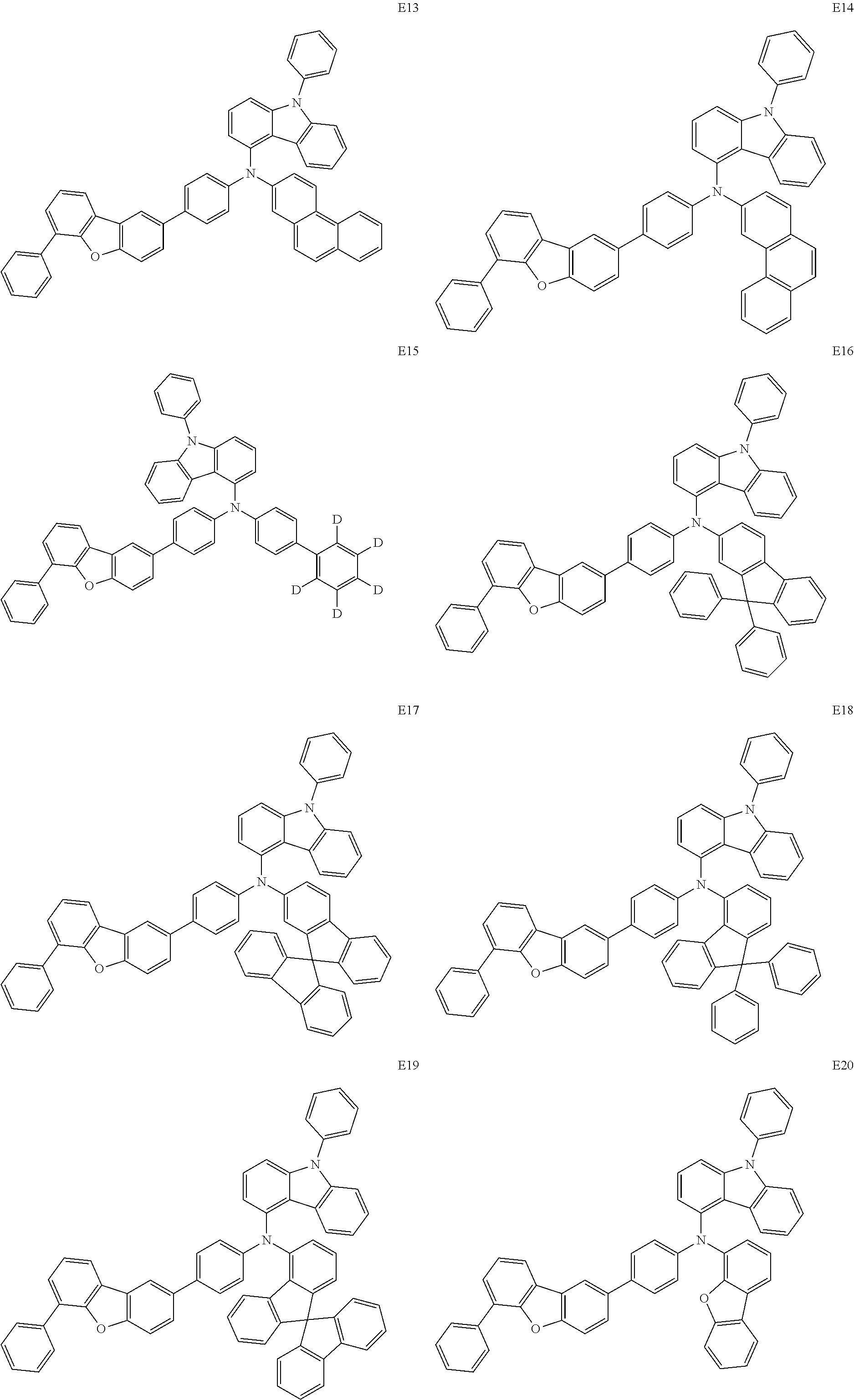

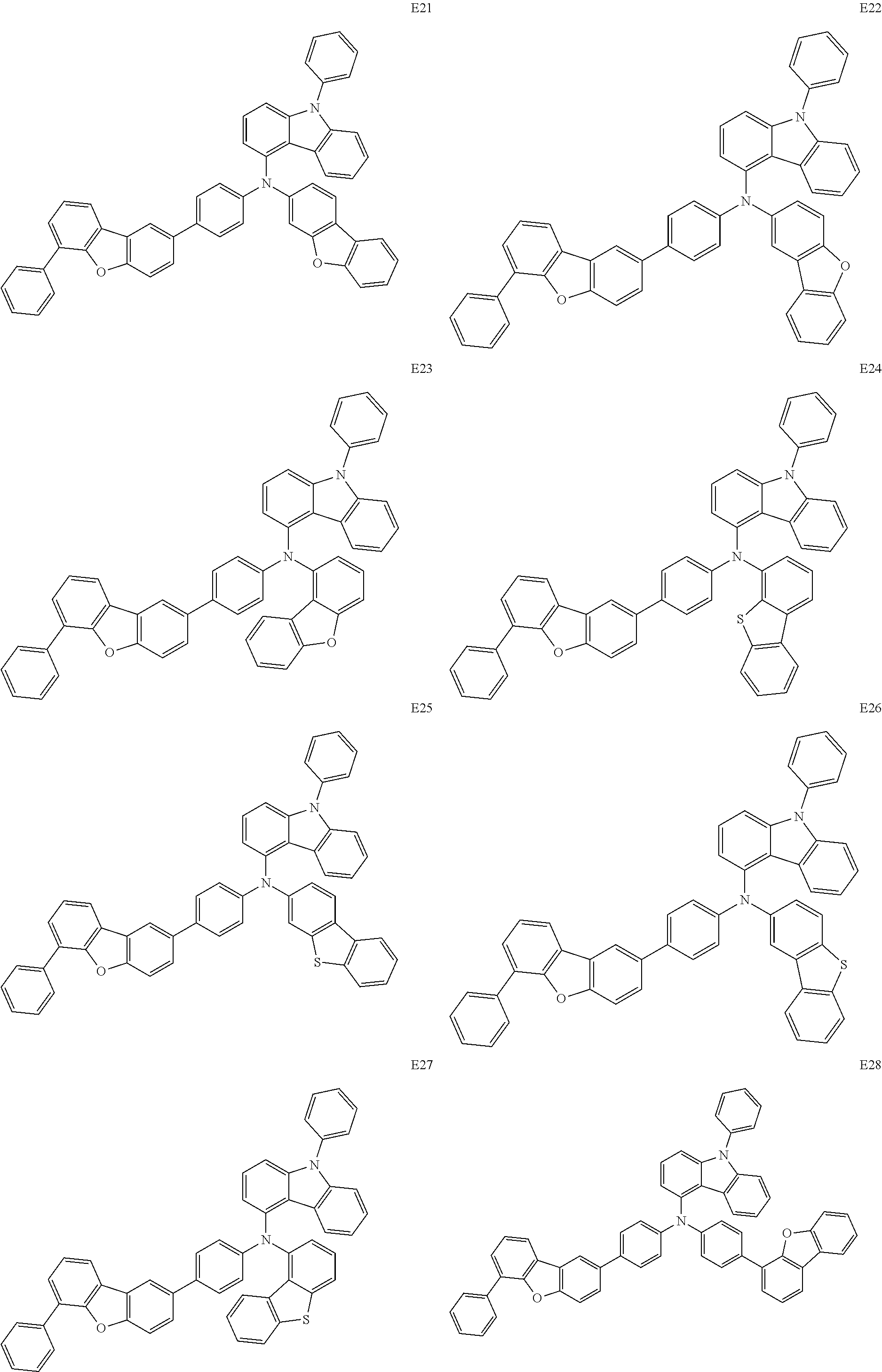

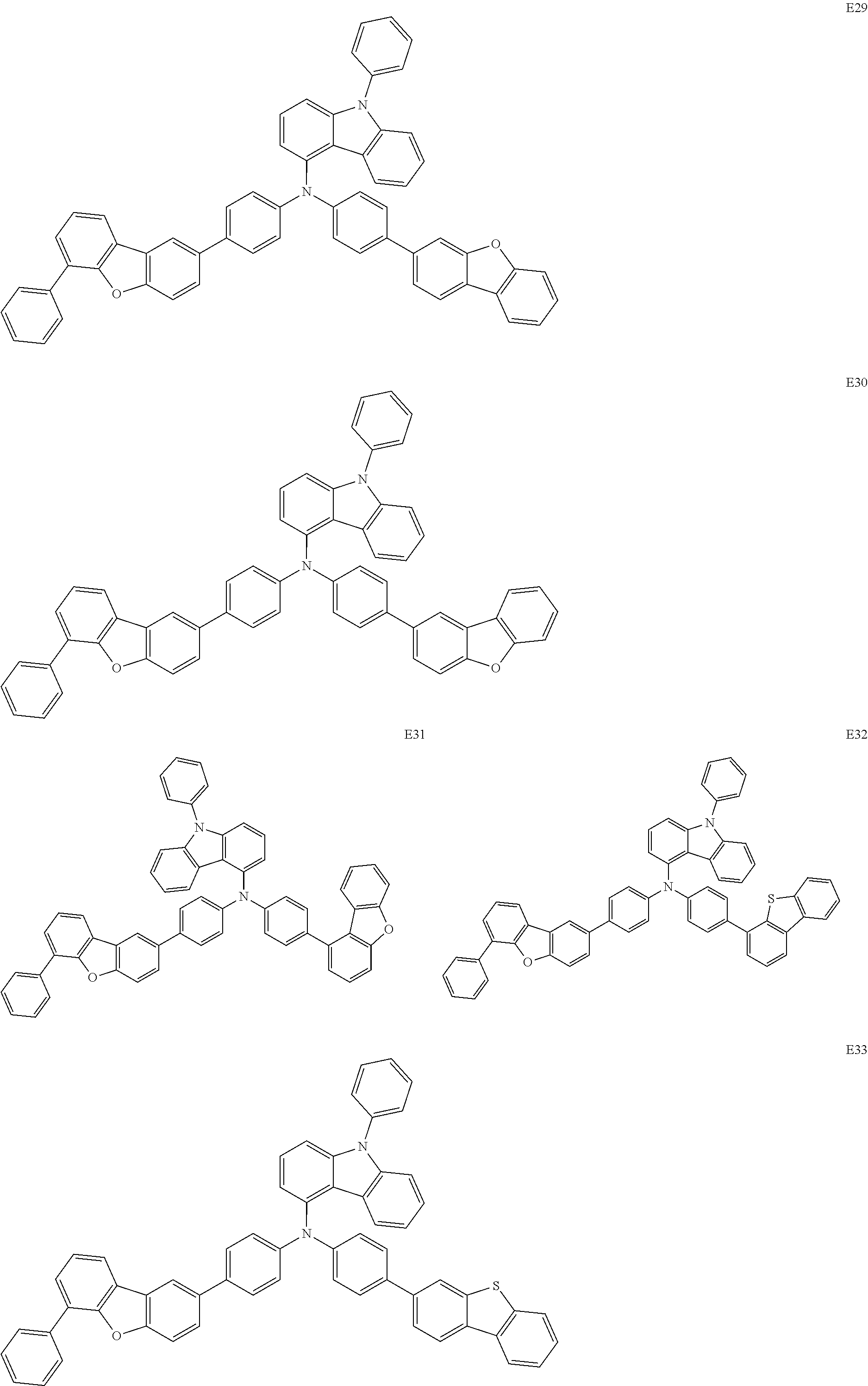

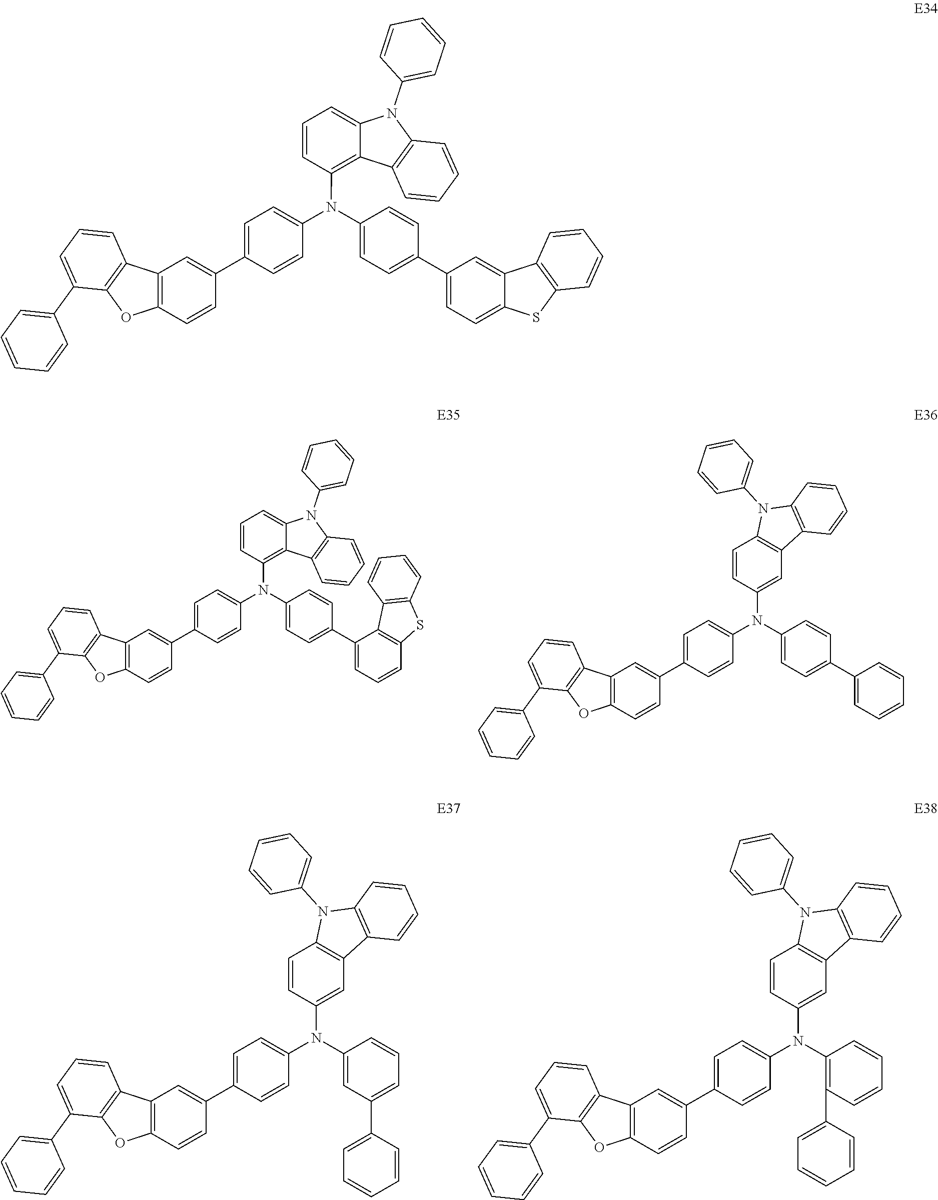

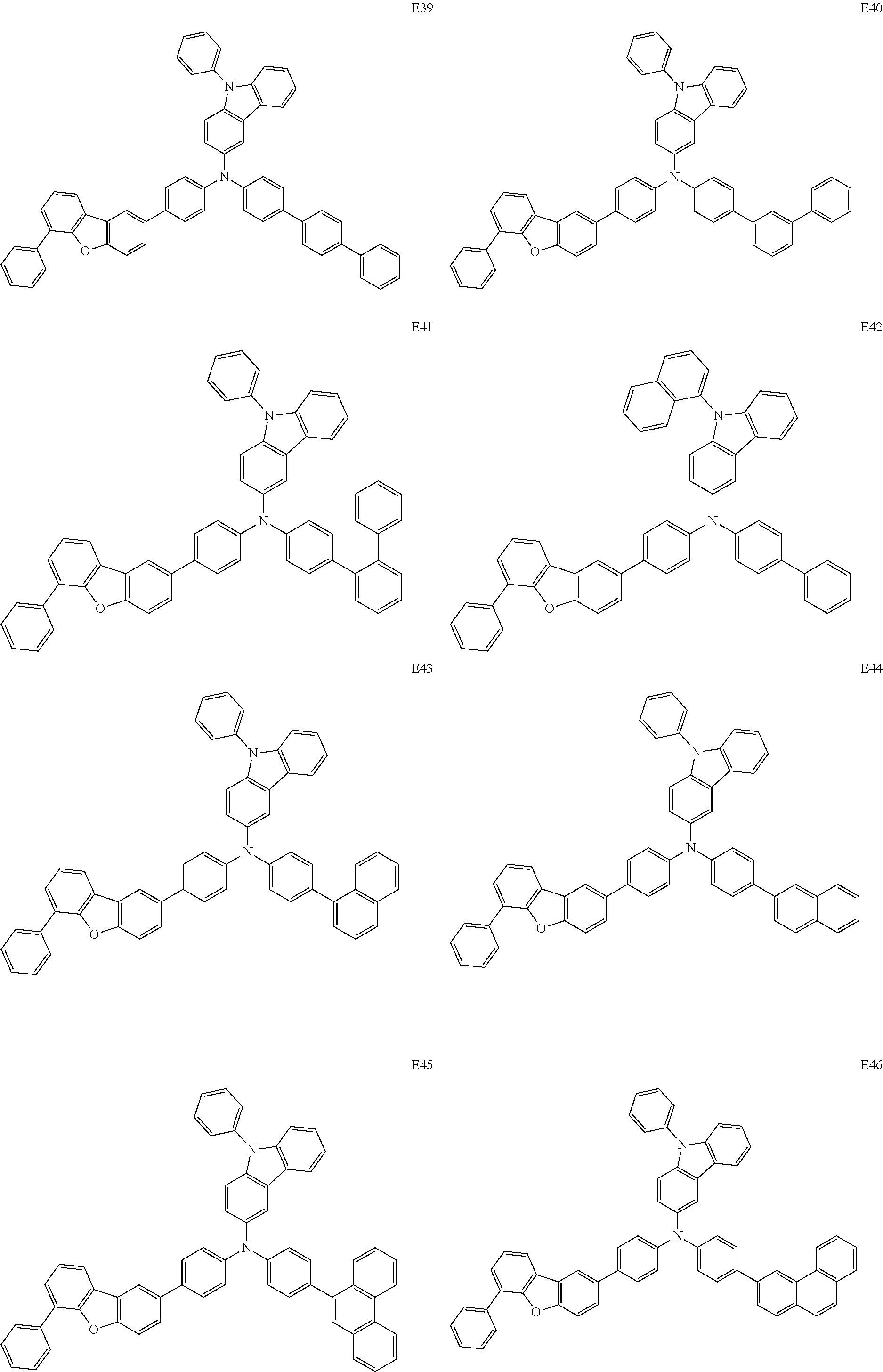

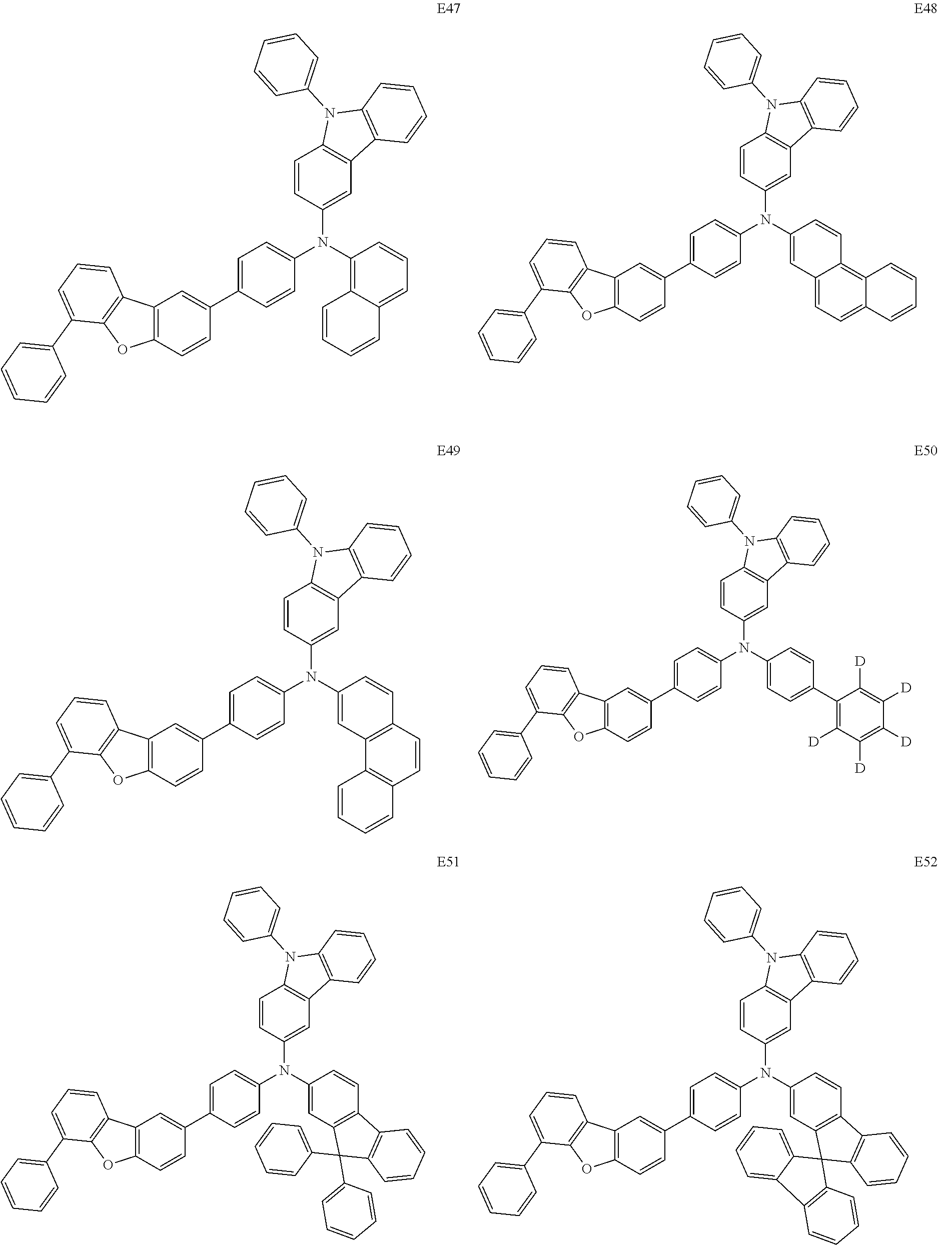

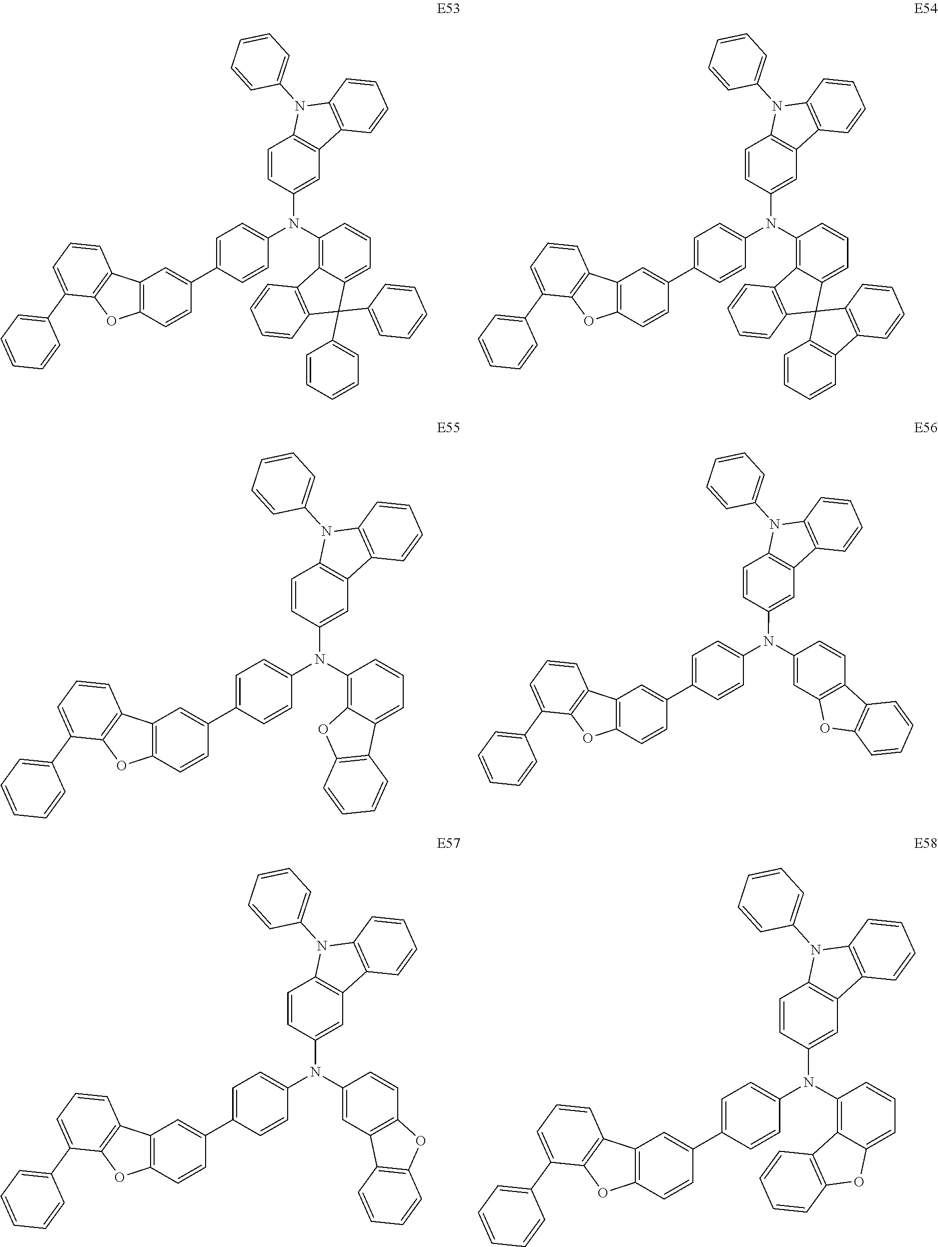

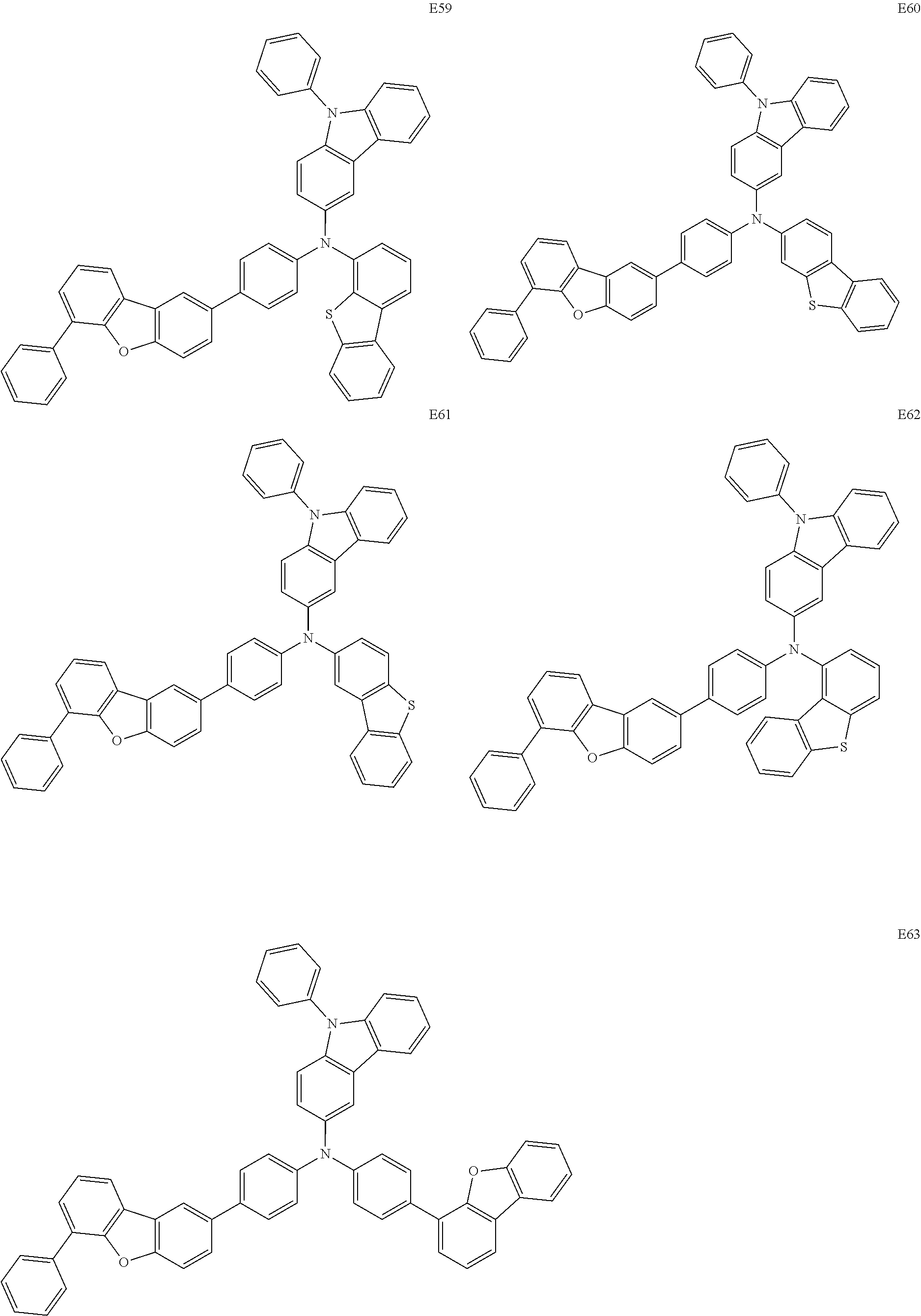 [SMILES: c1ccc(-c2cccc3c2oc2ccc(-c4ccc(N(c5ccc6c(c5)c5ccccc5n6-c5ccccc5)c5cccc6c5sc5ccccc56)cc4)cc23)cc1 ; c1ccc(-c2cccc3c2oc2ccc(-c4ccc(N(c5ccc6c(c5)sc5ccccc56)c5ccc6c(c5)c5ccccc5n6-c5ccccc5)cc4)cc23)cc1 ; c1ccc(-c2cccc3c2oc2ccc(-c4ccc(N(c5ccc6sc7ccccc7c6c5)c5ccc6c(c5)c5ccccc5n6-c5ccccc5)cc4)cc23)cc1 ; c1ccc(-c2cccc3c2oc2ccc(-c4ccc(N(c5ccc6c(c5)c5ccccc5n6-c5ccccc5)c5cccc6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc6c(c5)c5ccccc5n6-c5ccccc5)cc4)cc23)cc1]

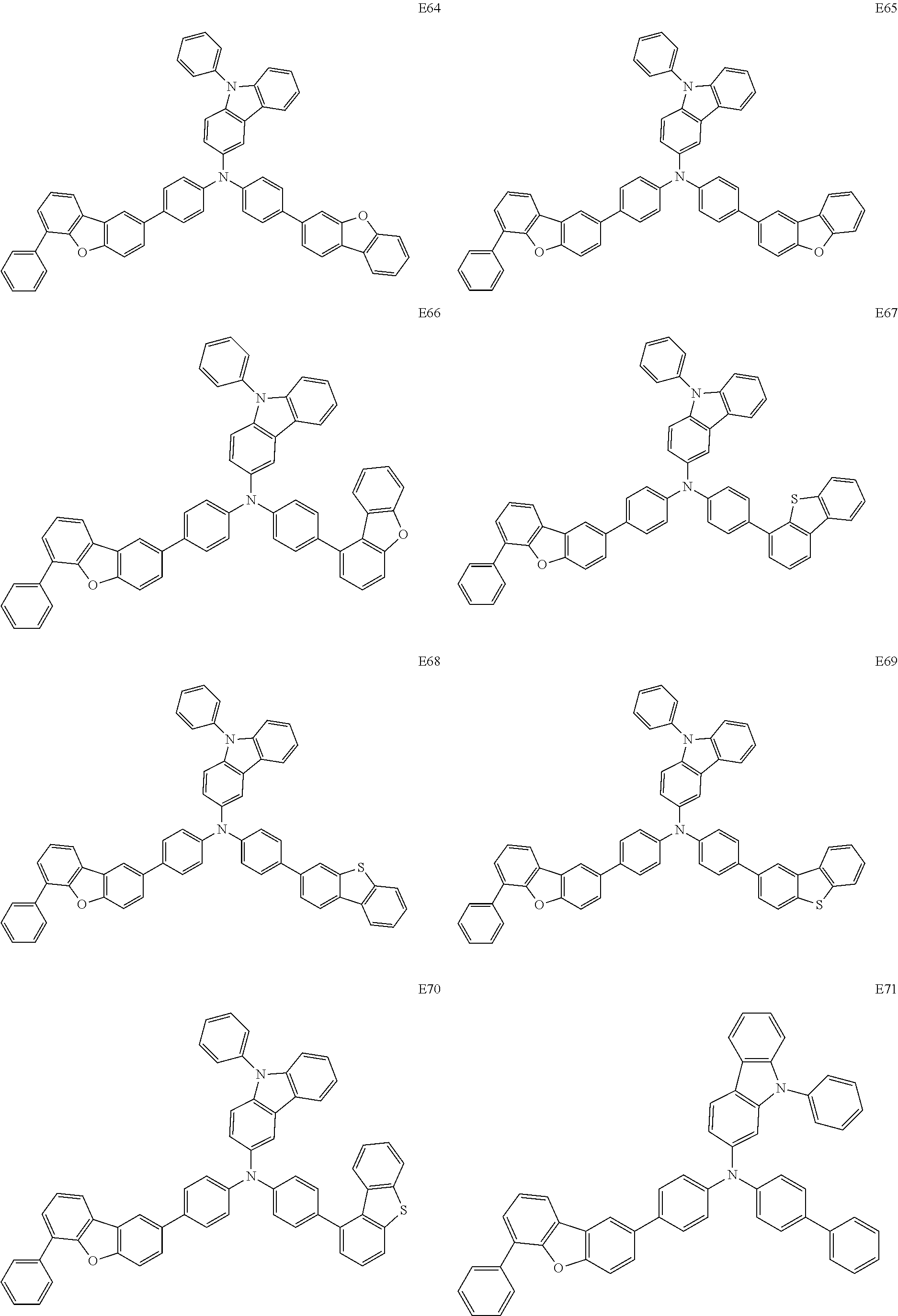

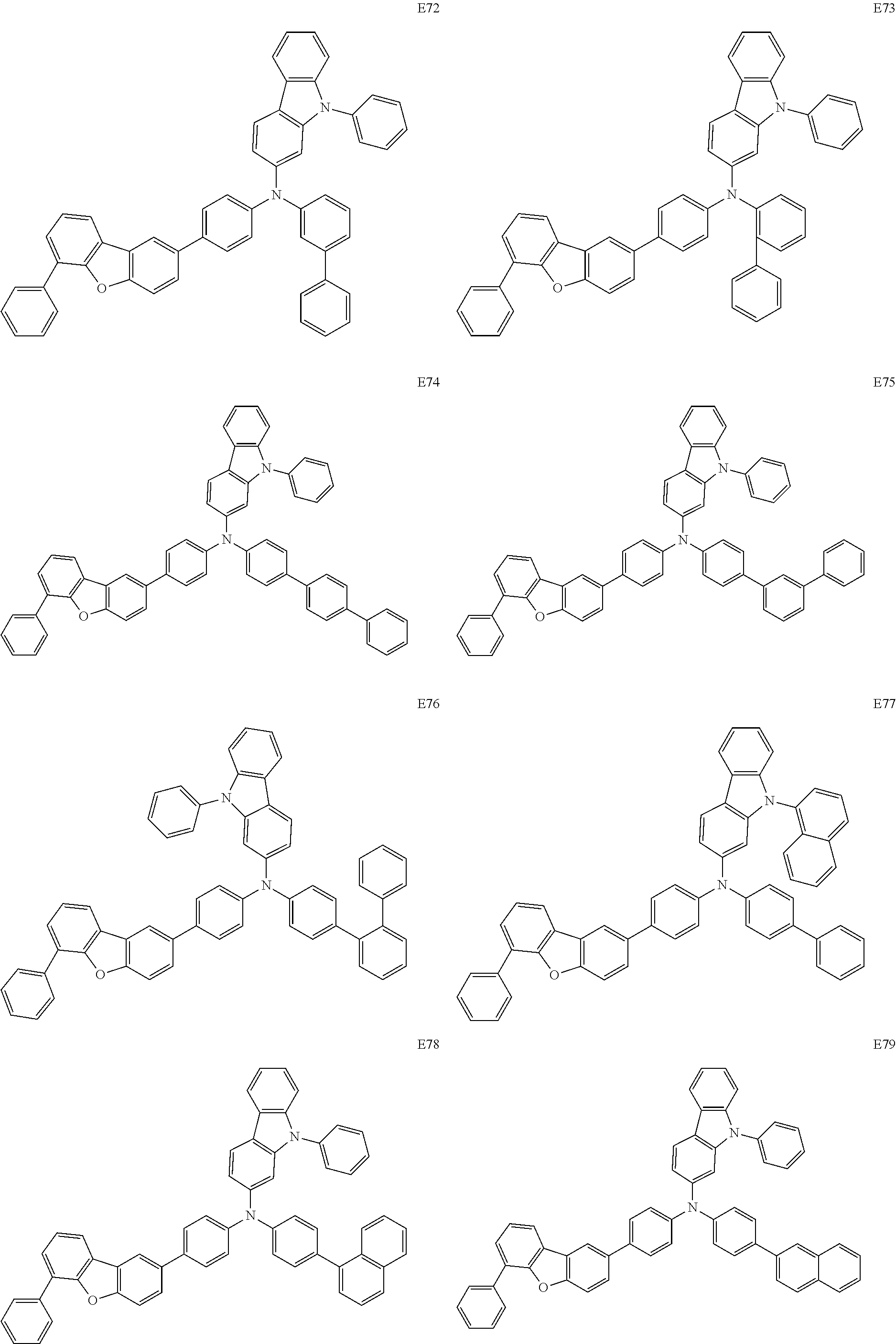

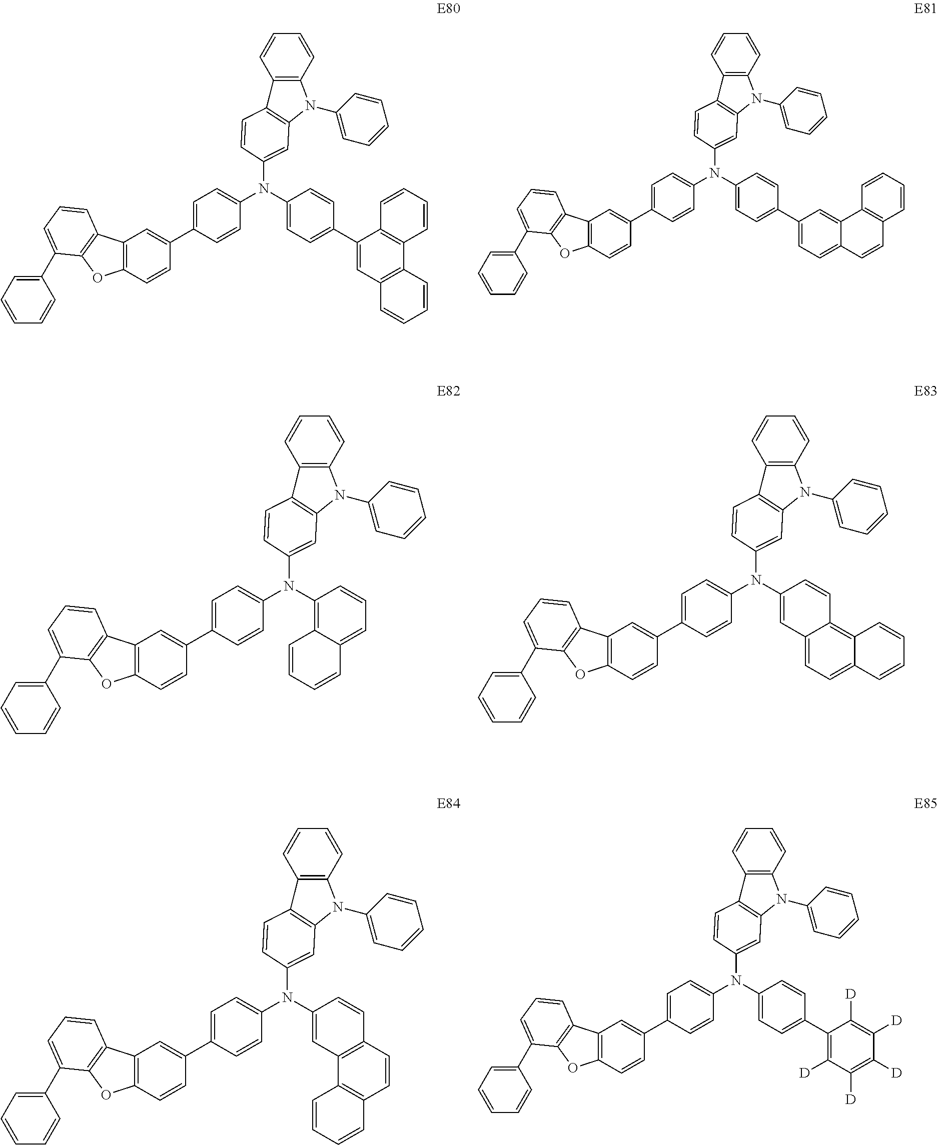

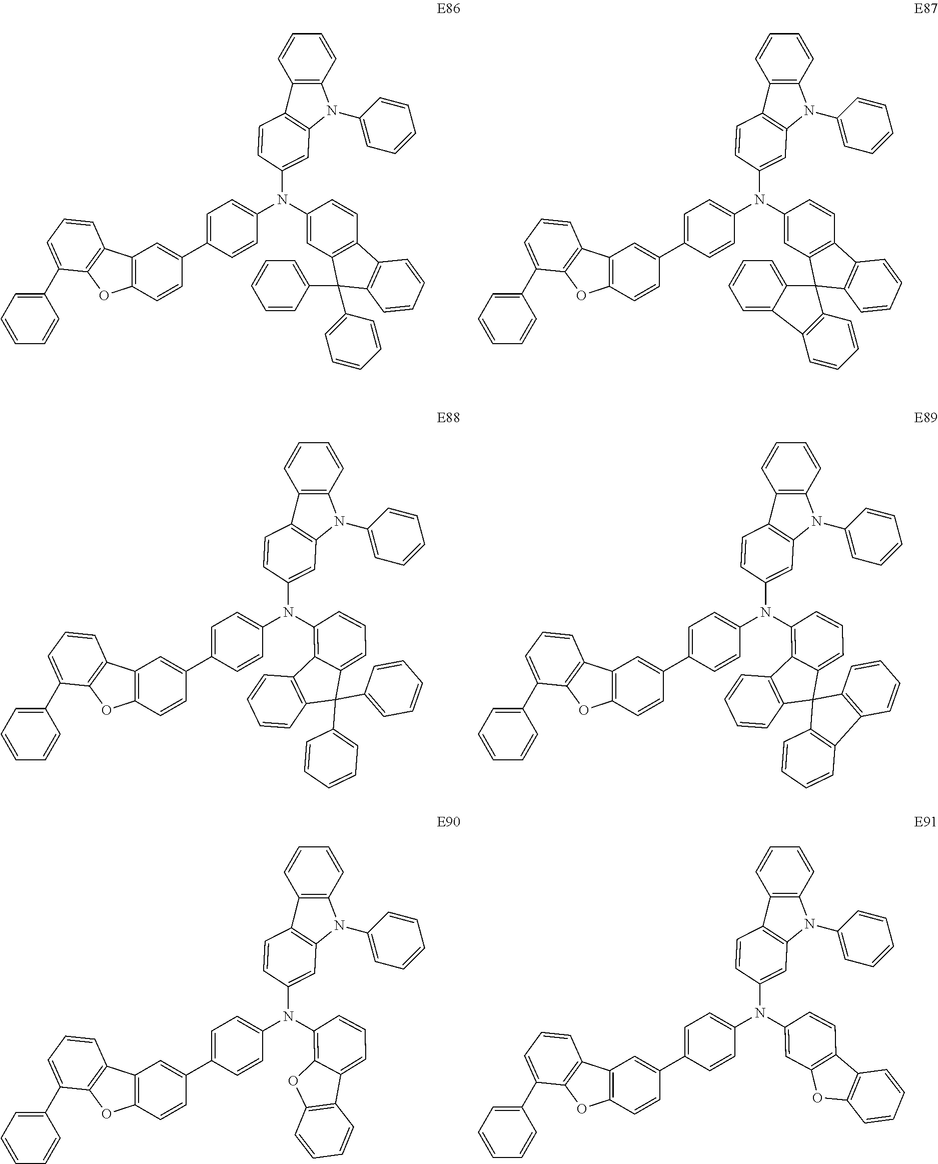

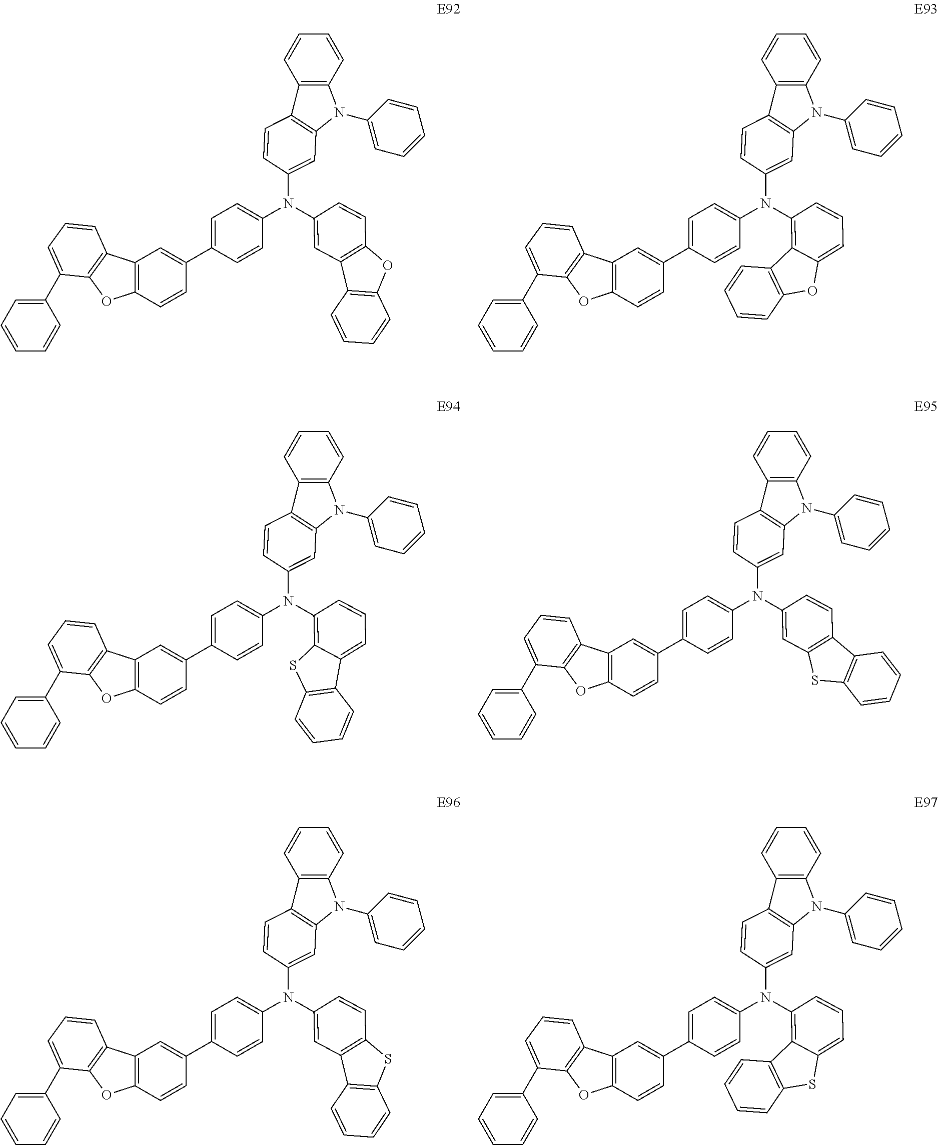

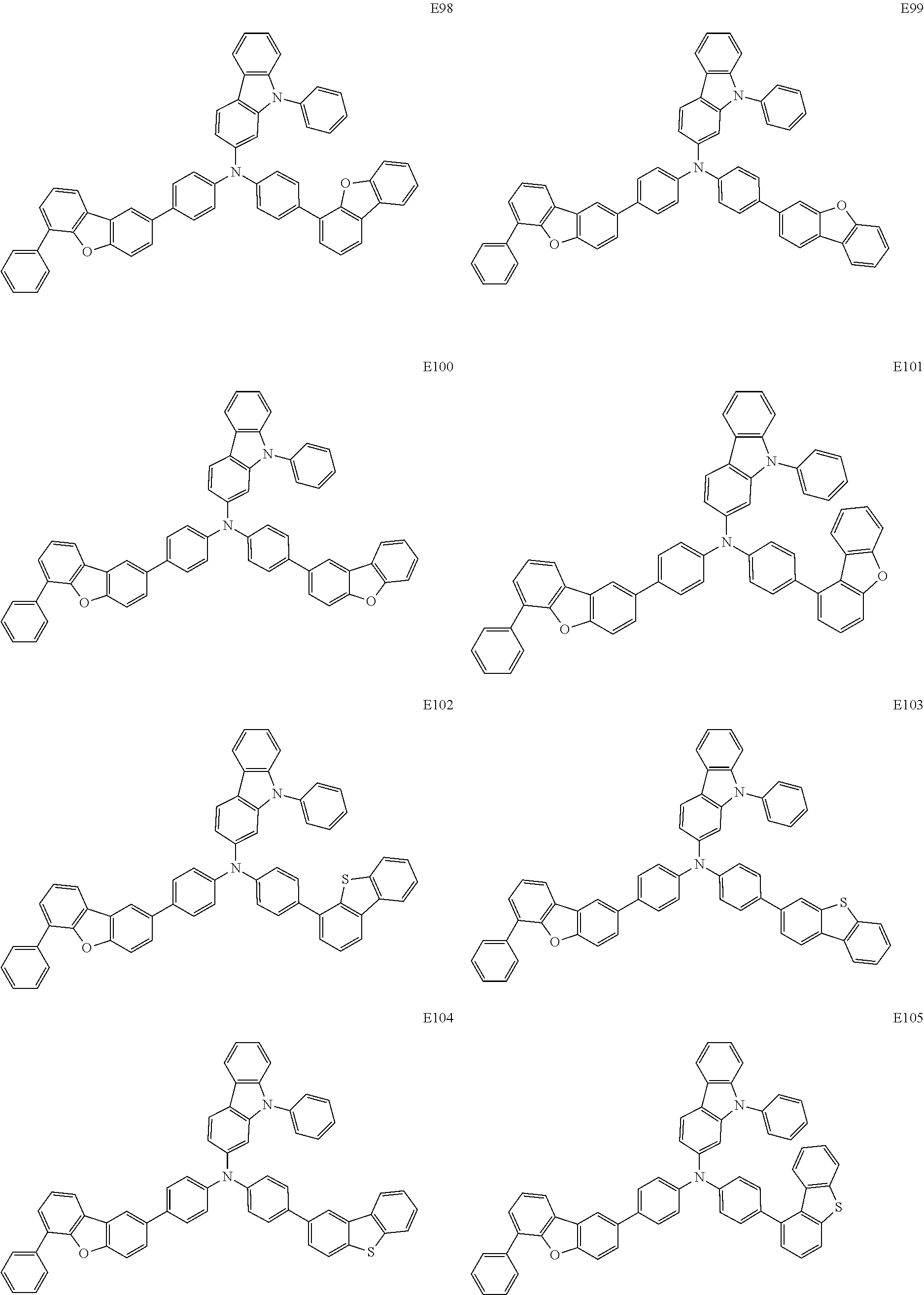

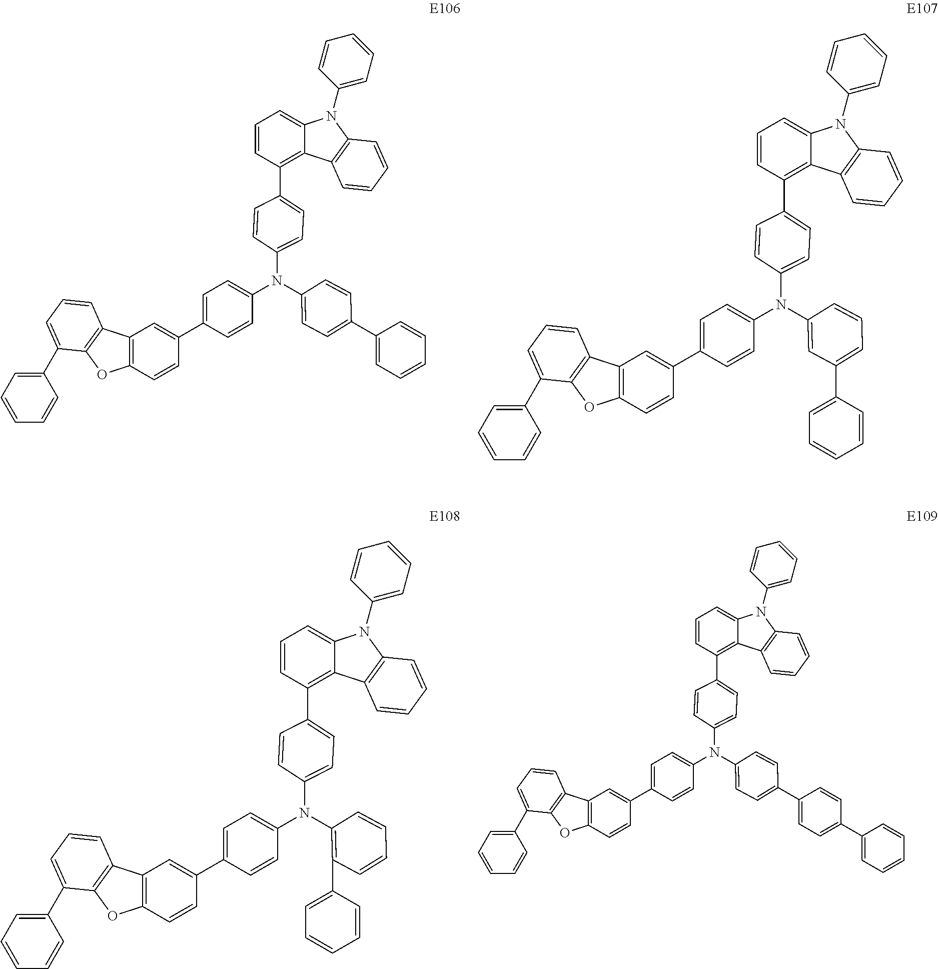 [SMILES: c1ccc(-c2ccc(N(c3ccc(-c4ccc5oc6c(-c7ccccc7)cccc6c5c4)cc3)c3ccc(-c4cccc5c4c4ccccc4n5-c4ccccc4)cc3)cc2)cc1 ; c1ccc(-c2cccc(N(c3ccc(-c4ccc5oc6c(-c7ccccc7)cccc6c5c4)cc3)c3ccc(-c4cccc5c4c4ccccc4n5-c4ccccc4)cc3)c2)cc1 ; c1ccc(-c2ccccc2N(c2ccc(-c3ccc4oc5c(-c6ccccc6)cccc5c4c3)cc2)c2ccc(-c3cccc4c3c3ccccc3n4-c3ccccc3)cc2)cc1 ; c1ccc(-c2ccc(-c3ccc(N(c4ccc(-c5ccc6oc7c(-c8ccccc8)cccc7c6c5)cc4)c4ccc(-c5cccc6c5c5ccccc5n6-c5ccccc5)cc4)cc3)cc2)cc1]

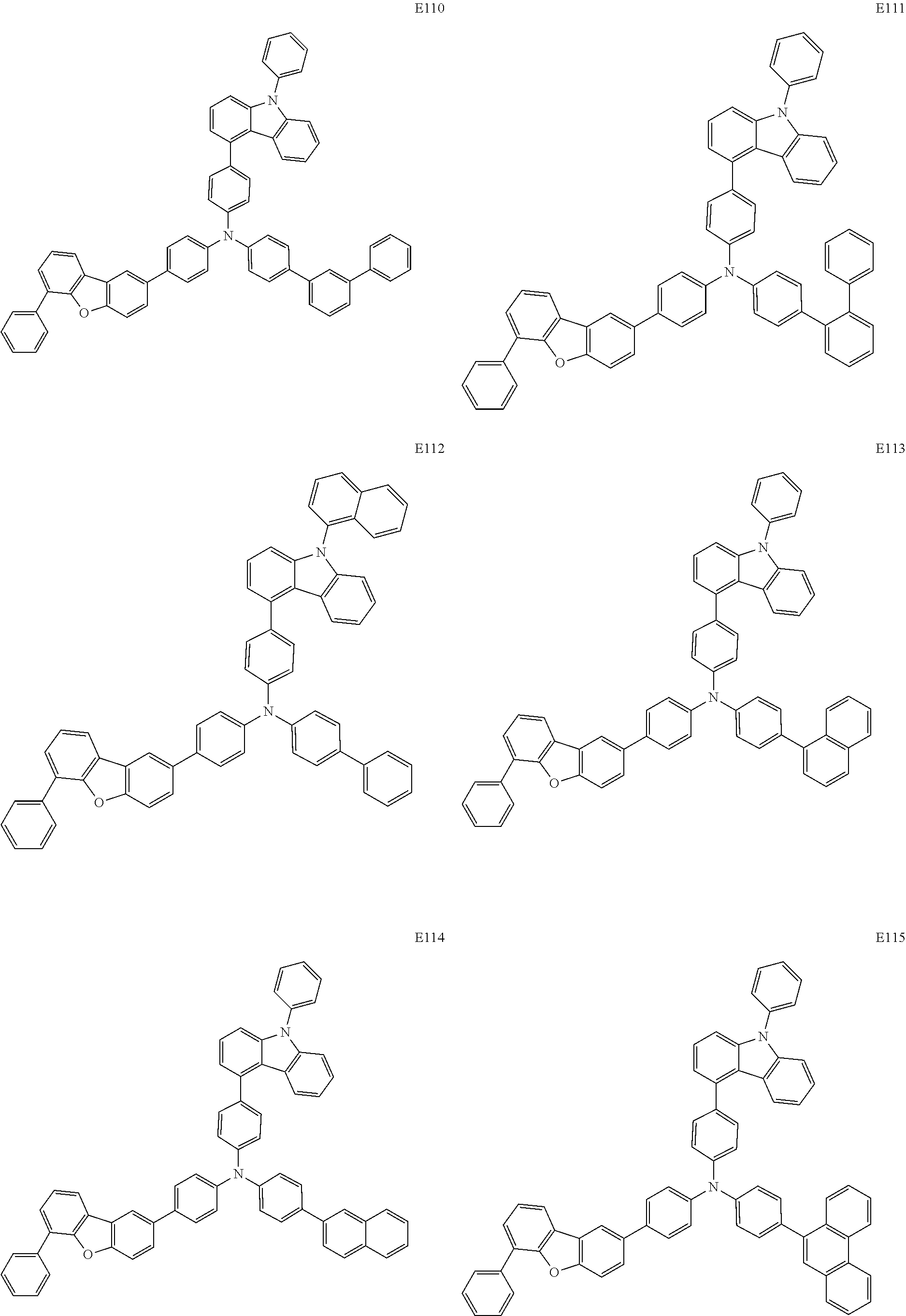

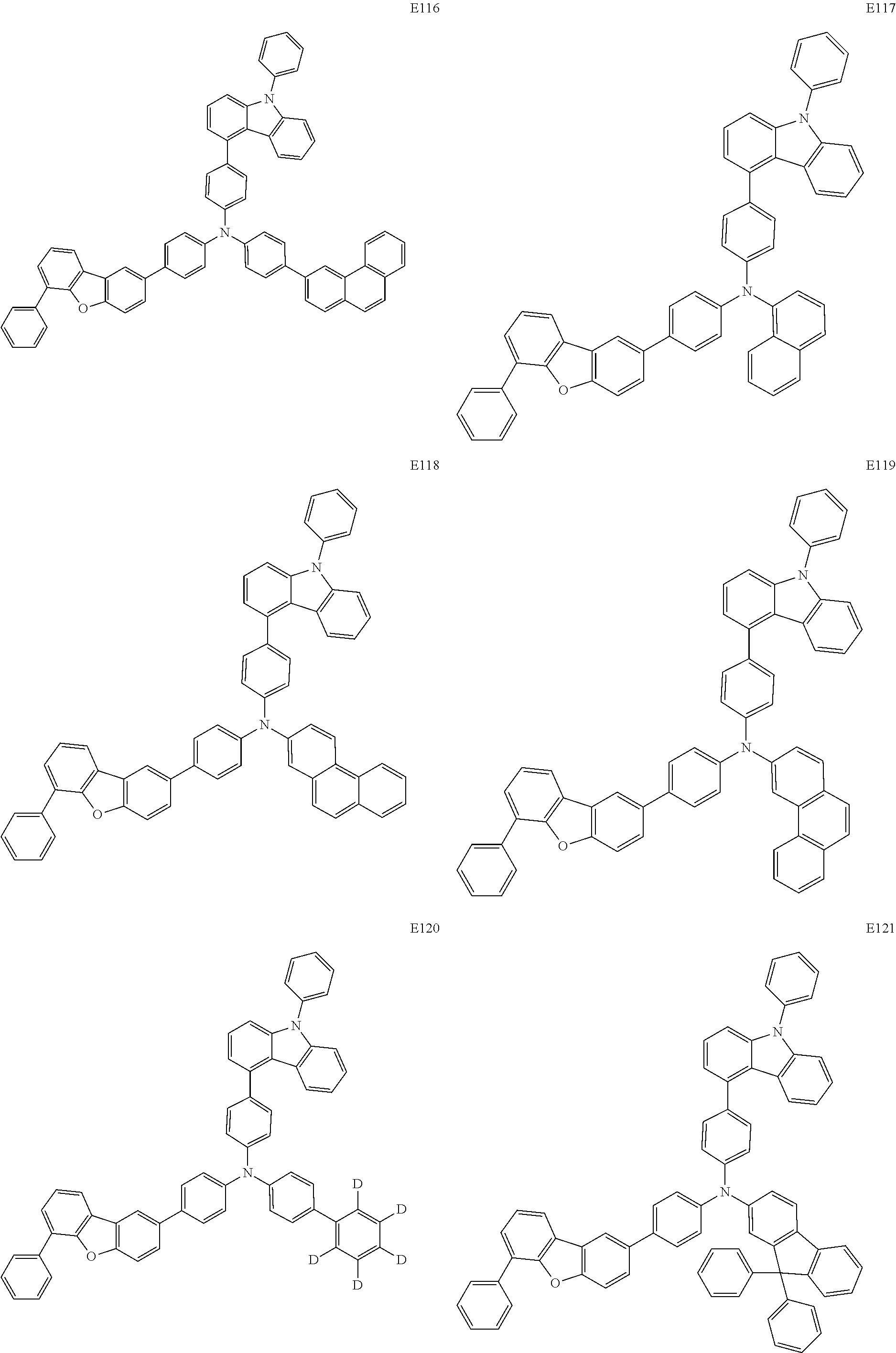

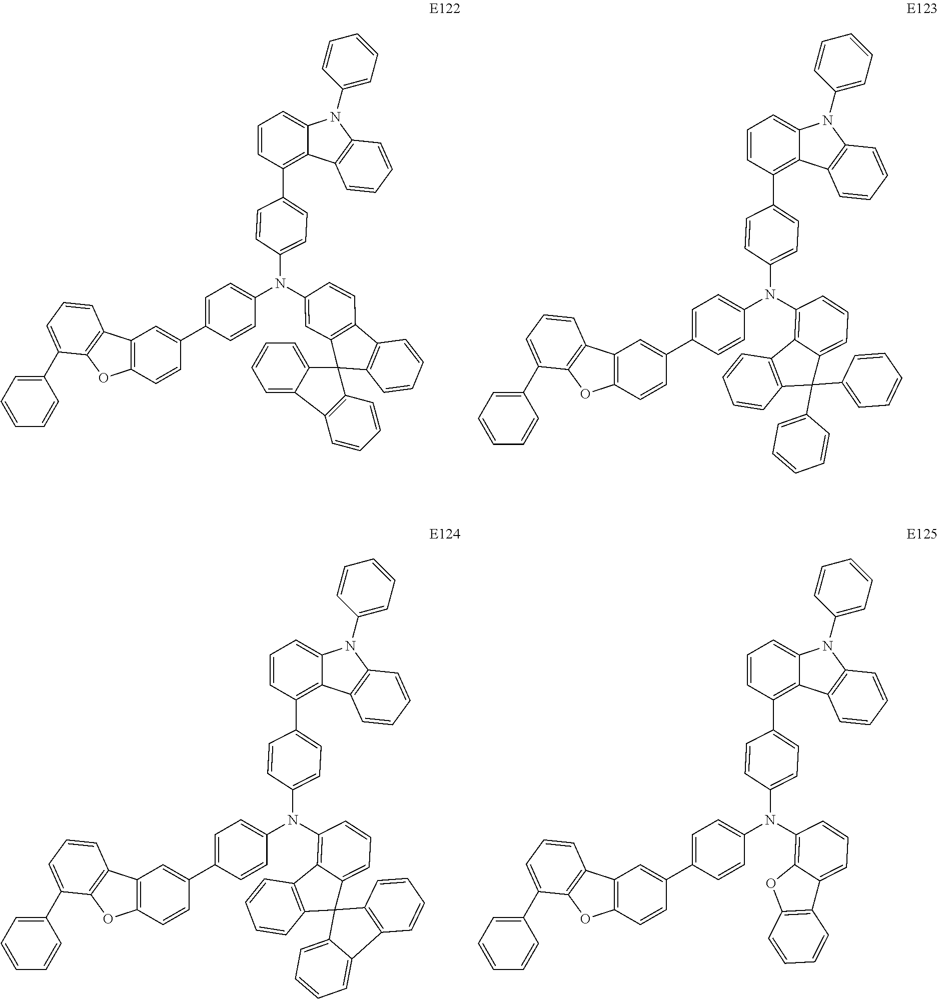 [SMILES: c1ccc(-c2cccc3c2oc2ccc(-c4ccc(N(c5ccc(-c6cccc7c6c6ccccc6n7-c6ccccc6)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6cccc7c6c6ccccc6n7-c6ccccc6)cc5)c5cccc6c5-c5ccccc5C6(c5ccccc5)c5ccccc5)cc4)cc23)cc1 ; c1ccc(-c2cccc3c2oc2ccc(-c4ccc(N(c5ccc(-c6cccc7c6c6ccccc6n7-c6ccccc6)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6cccc7c6c6ccccc6n7-c6ccccc6)cc5)c5cccc6c5oc5ccccc56)cc4)cc23)cc1]

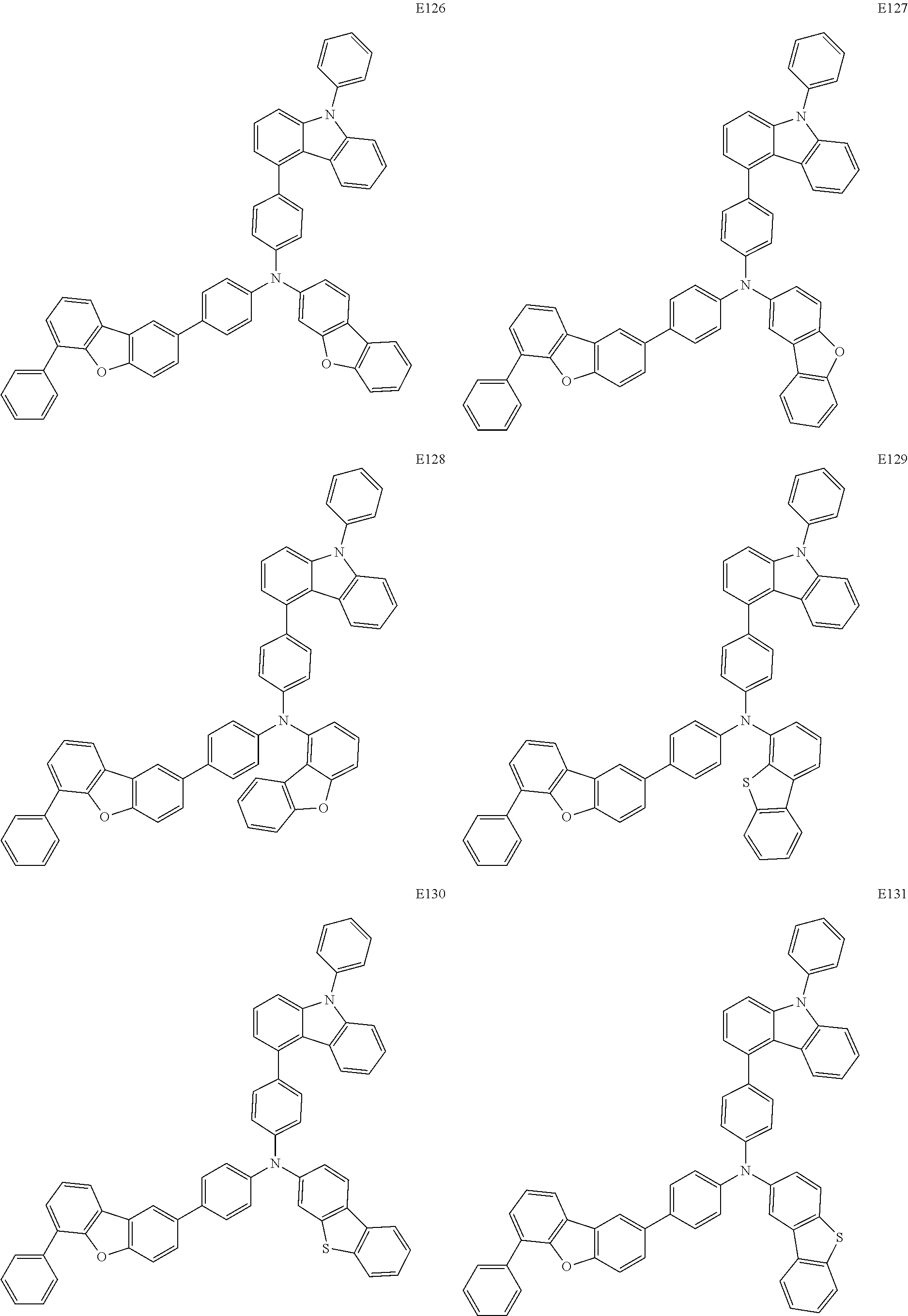

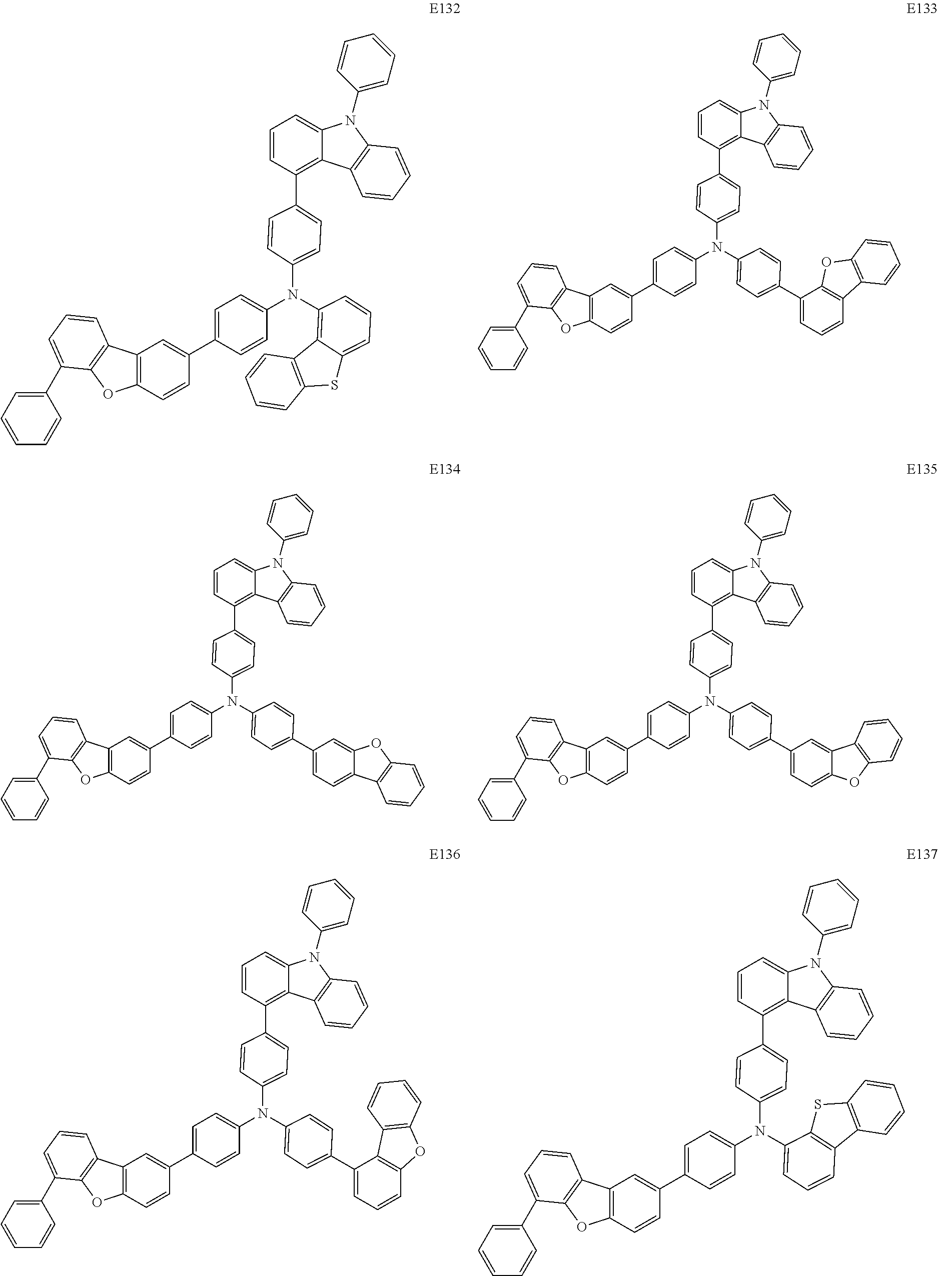

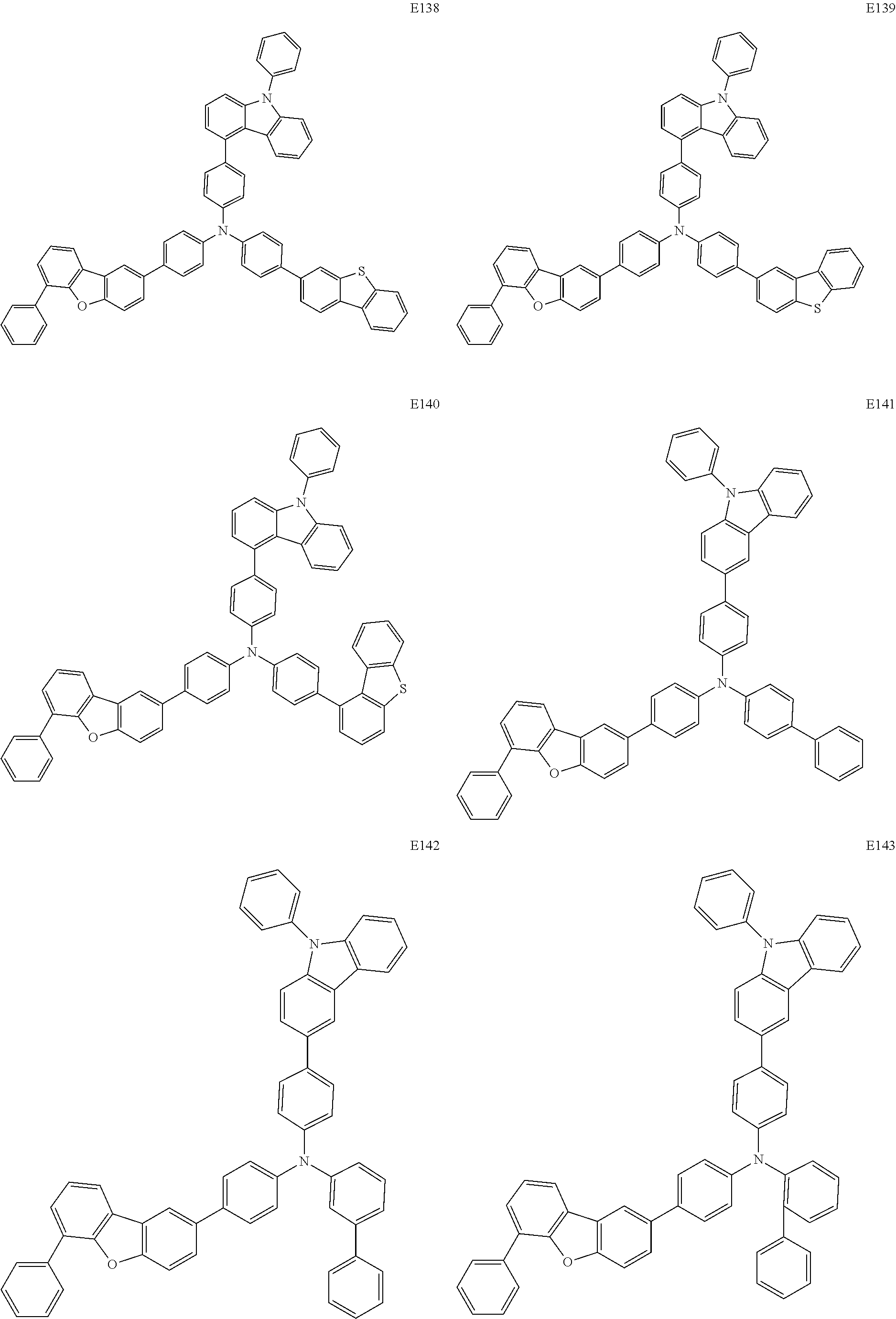

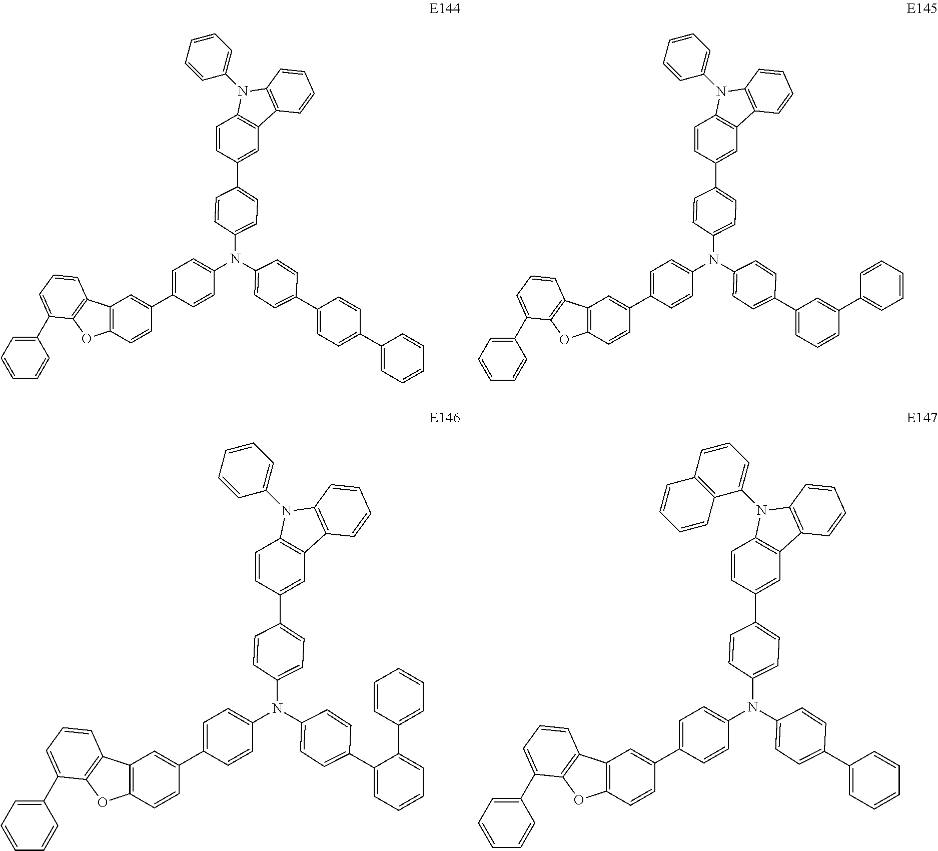 [SMILES: c1ccc(-c2ccc(-c3ccc(N(c4ccc(-c5ccc6oc7c(-c8ccccc8)cccc7c6c5)cc4)c4ccc(-c5ccc6c(c5)c5ccccc5n6-c5ccccc5)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5ccc6c(c5)c5ccccc5n6-c5ccccc5)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4ccc5c(c4)c4ccccc4n5-c4ccccc4)cc3)cc2)cc1 ; c1ccc(-c2ccc(N(c3ccc(-c4ccc5oc6c(-c7ccccc7)cccc6c5c4)cc3)c3ccc(-c4ccc5c(c4)c4ccccc4n5-c4cccc5ccccc45)cc3)cc2)cc1]

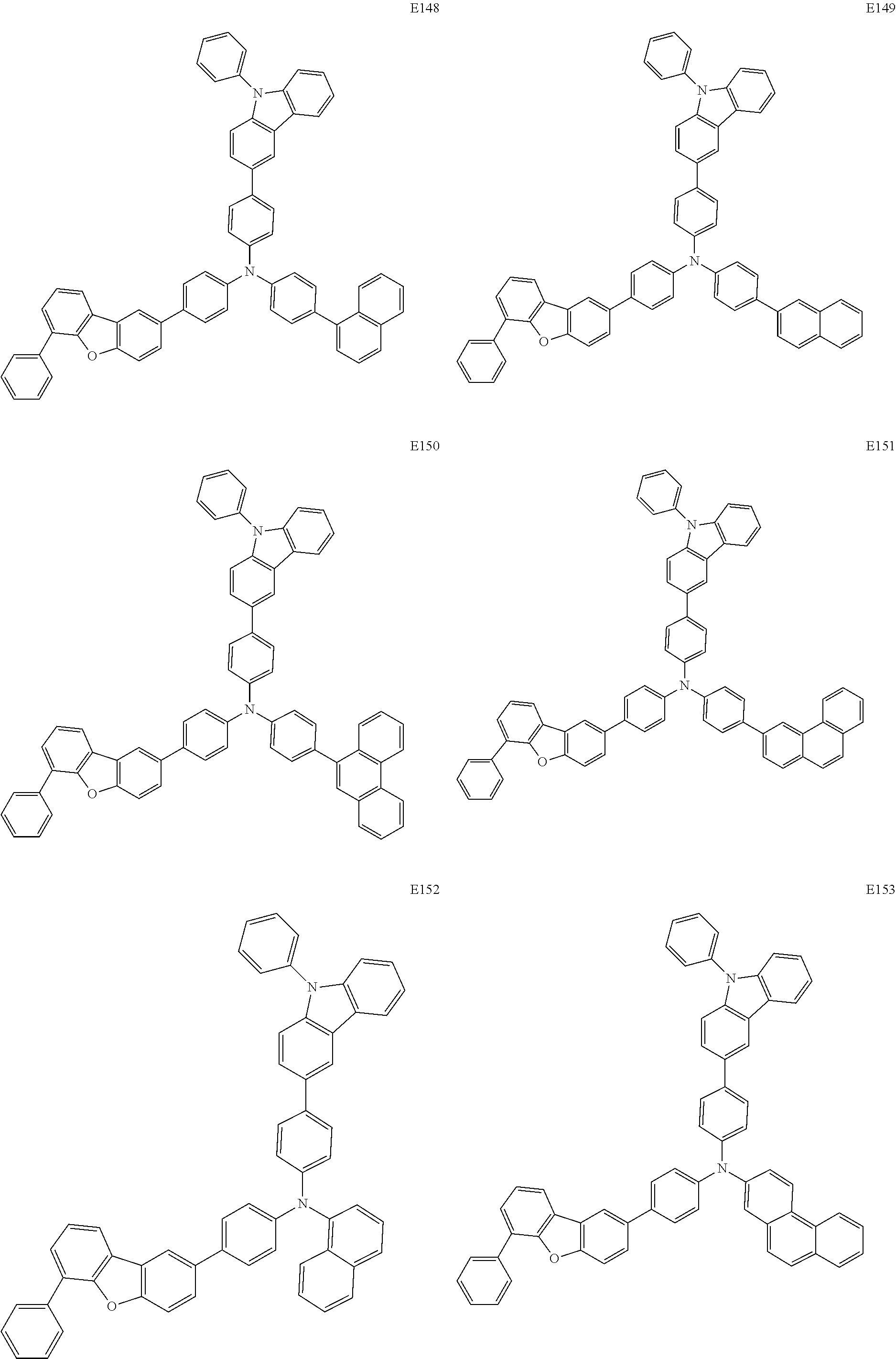

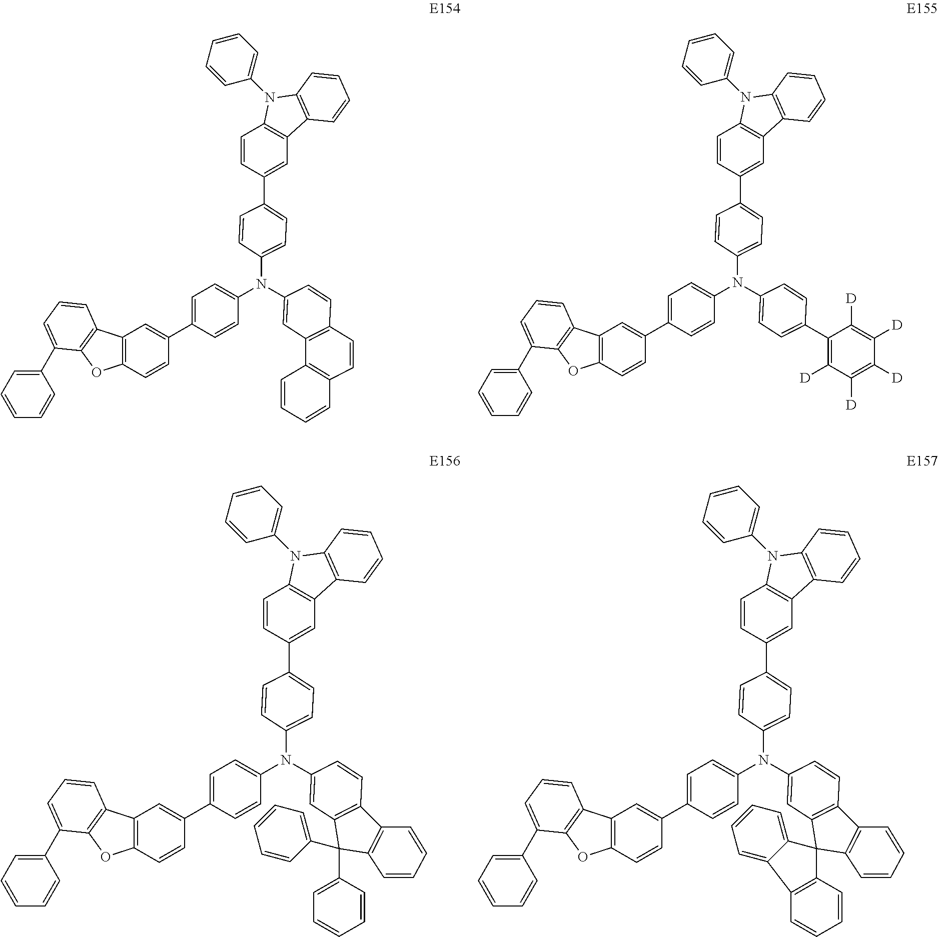

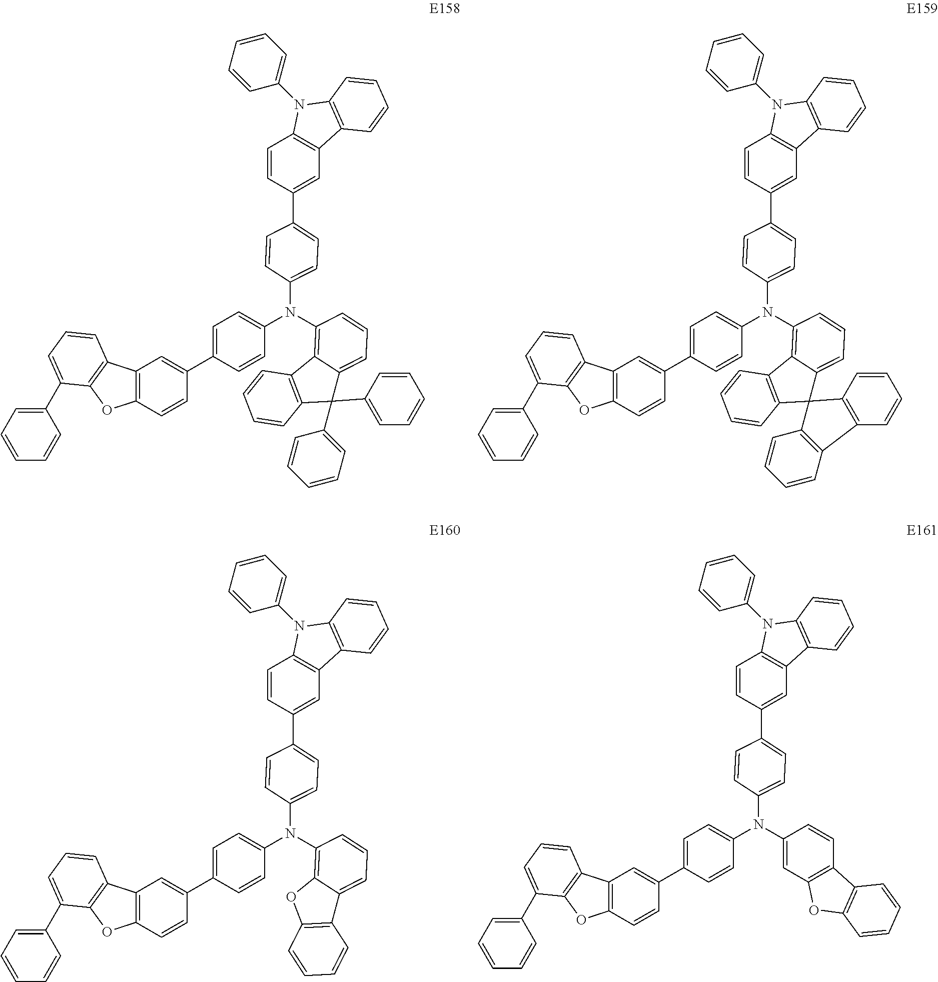 [SMILES: c1ccc(-c2cccc3c2oc2ccc(-c4ccc(N(c5ccc(-c6ccc7c(c6)c6ccccc6n7-c6ccccc6)cc5)c5cccc6c5-c5ccccc5C6(c5ccccc5)c5ccccc5)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6c5oc5ccccc56)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6c(c5)oc5ccccc56)cc4)cc23)cc1]

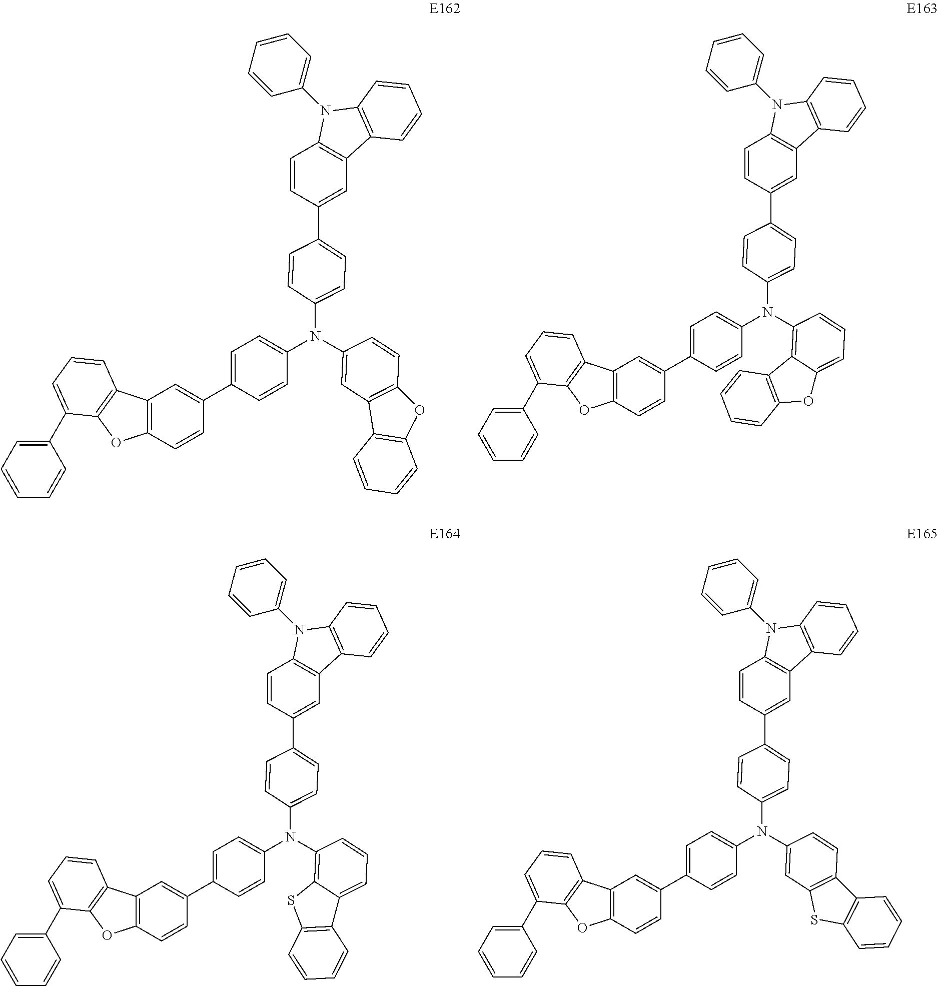 [SMILES: c1ccc(-c2cccc3c2oc2ccc(-c4ccc(N(c5ccc(-c6ccc7c(c6)c6ccccc6n7-c6ccccc6)cc5)c5ccc6oc7ccccc7c6c5)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6oc7ccccc7c56)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6c5sc5ccccc56)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6c(c5)sc5ccccc56)cc4)cc23)cc1]

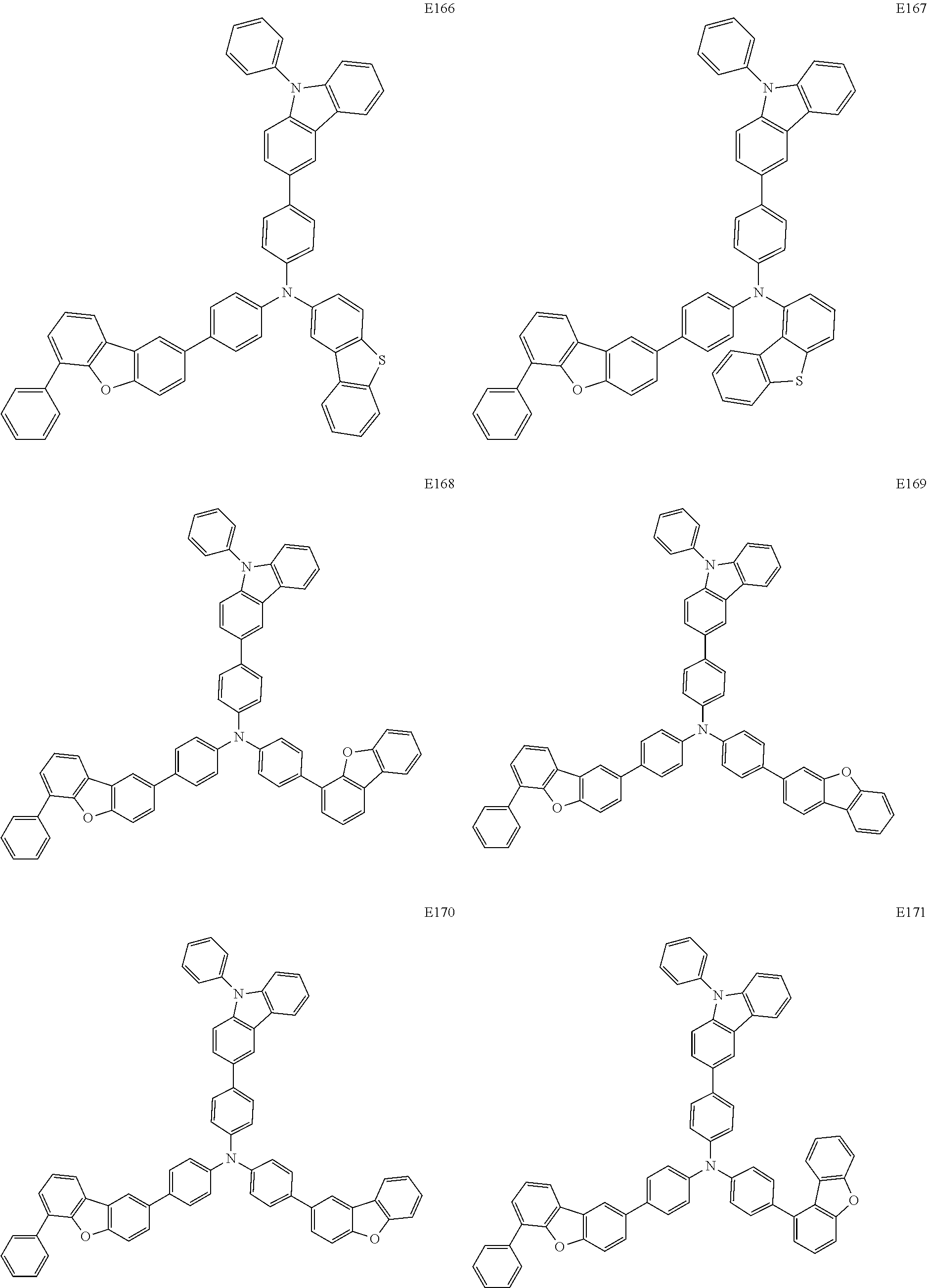

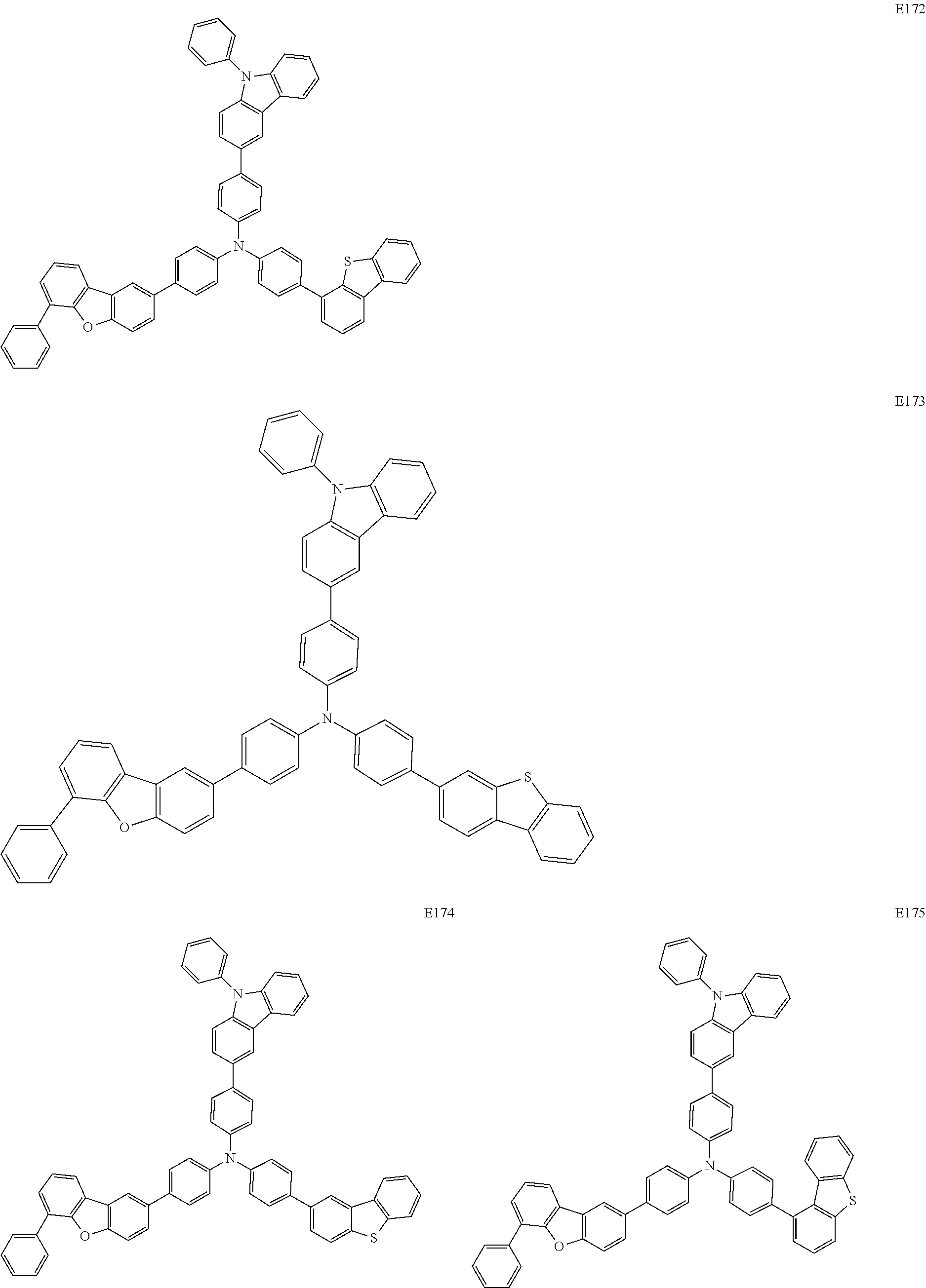 [SMILES: c1ccc(-c2cccc3c2oc2ccc(-c4ccc(N(c5ccc(-c6ccc7c(c6)c6ccccc6n7-c6ccccc6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6ccc7c(c6)c6ccccc6n7-c6ccccc6)cc5)cc4)cc23)cc1 ; c1ccc(-c2cccc3c2oc2ccc(-c4ccc(N(c5ccc(-c6ccc7sc8ccccc8c7c6)cc5)c5ccc(-c6ccc7c(c6)c6ccccc6n7-c6ccccc6)cc5)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc(-c6cccc7sc8ccccc8c67)cc5)cc4)cc23)cc1]

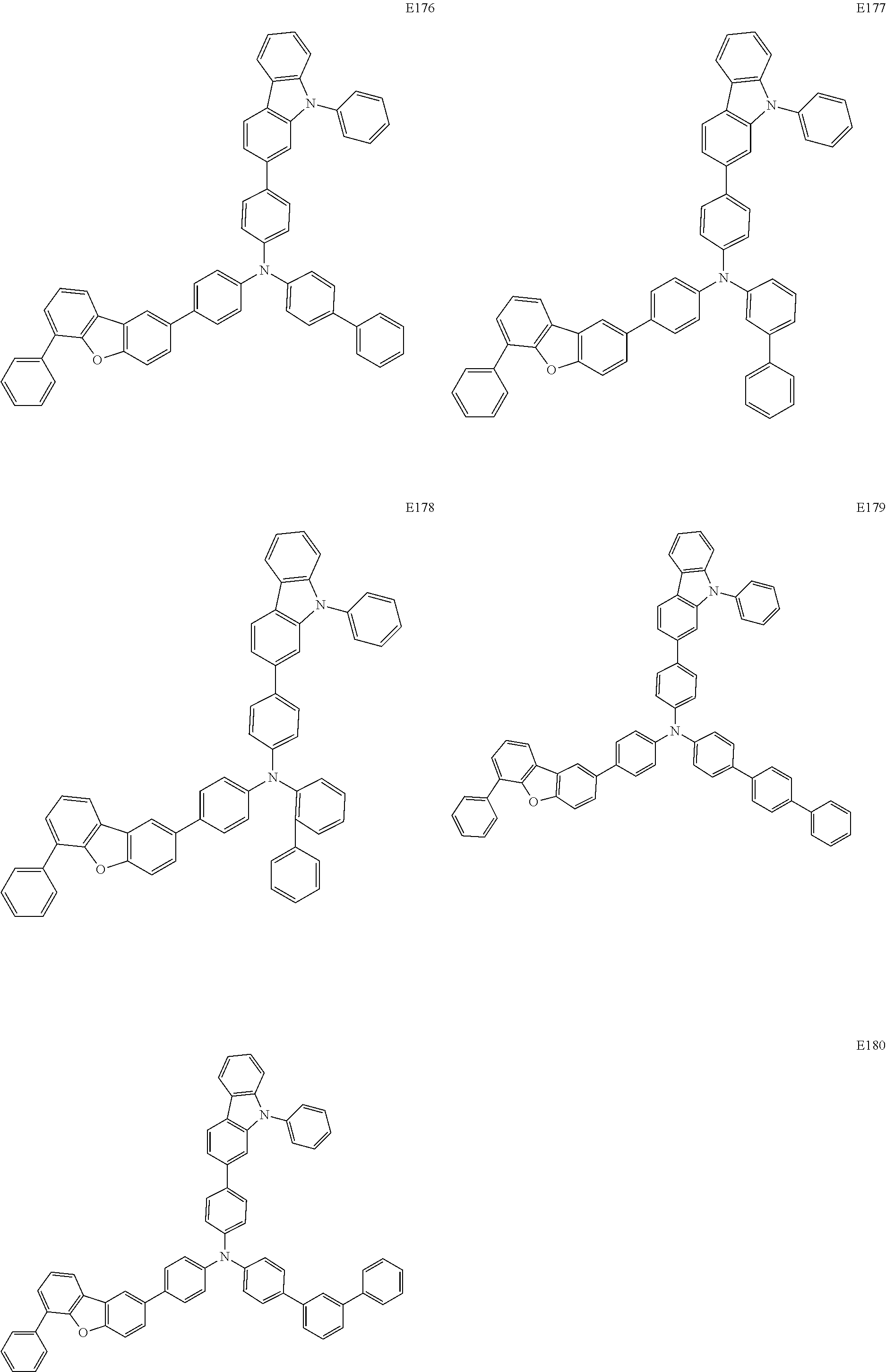

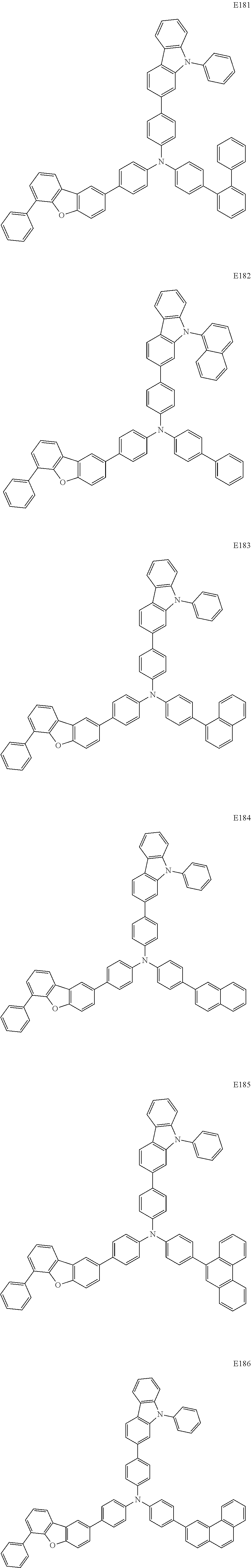

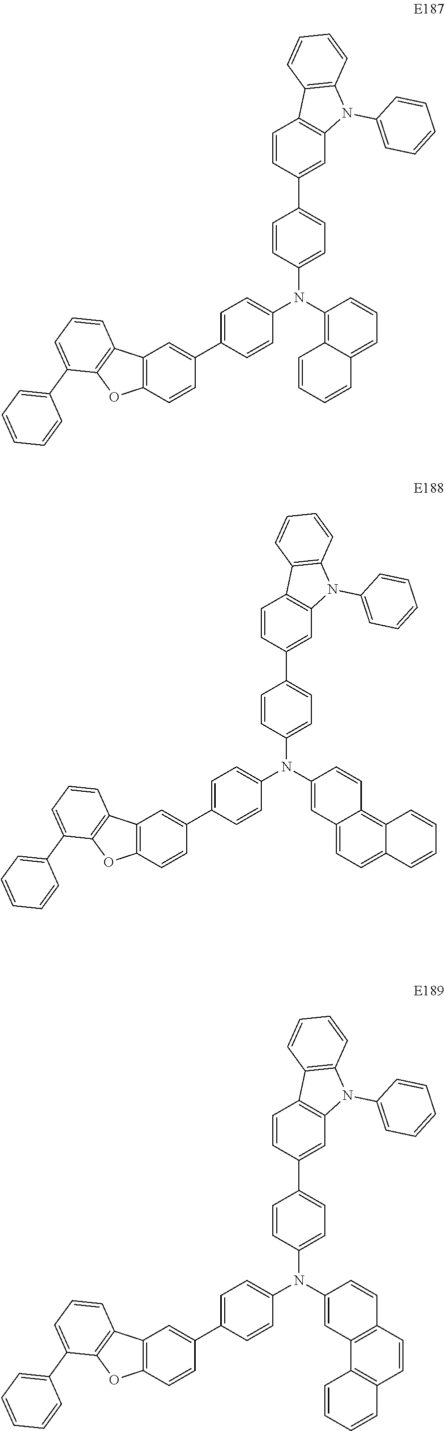 [SMILES: c1ccc(-c2cccc3c2oc2ccc(-c4ccc(N(c5ccc(-c6ccc7c8ccccc8n(-c8ccccc8)c7c6)cc5)c5cccc6ccccc56)cc4)cc23)cc1 ; c1ccc(-c2cccc3c2oc2ccc(-c4ccc(N(c5ccc(-c6ccc7c8ccccc8n(-c8ccccc8)c7c6)cc5)c5ccc6c(ccc7ccccc76)c5)cc4)cc23)cc1 ; c1ccc(-c2cccc3c2oc2ccc(-c4ccc(N(c5ccc(-c6ccc7c8ccccc8n(-c8ccccc8)c7c6)cc5)c5ccc6ccc7ccccc7c6c5)cc4)cc23)cc1]

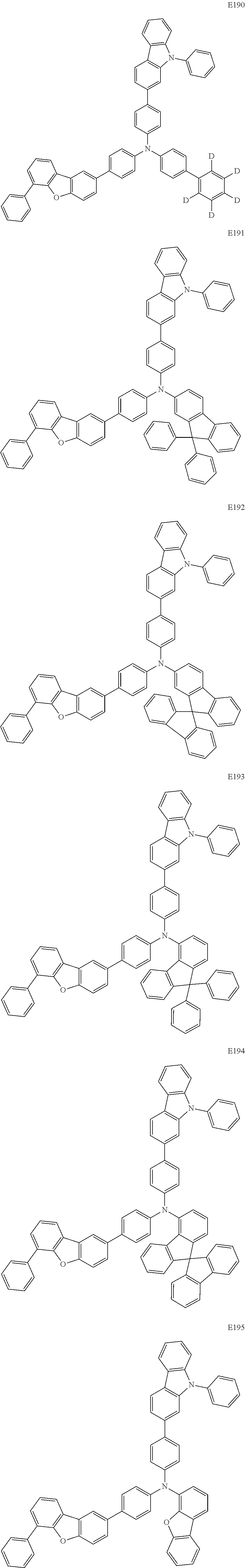

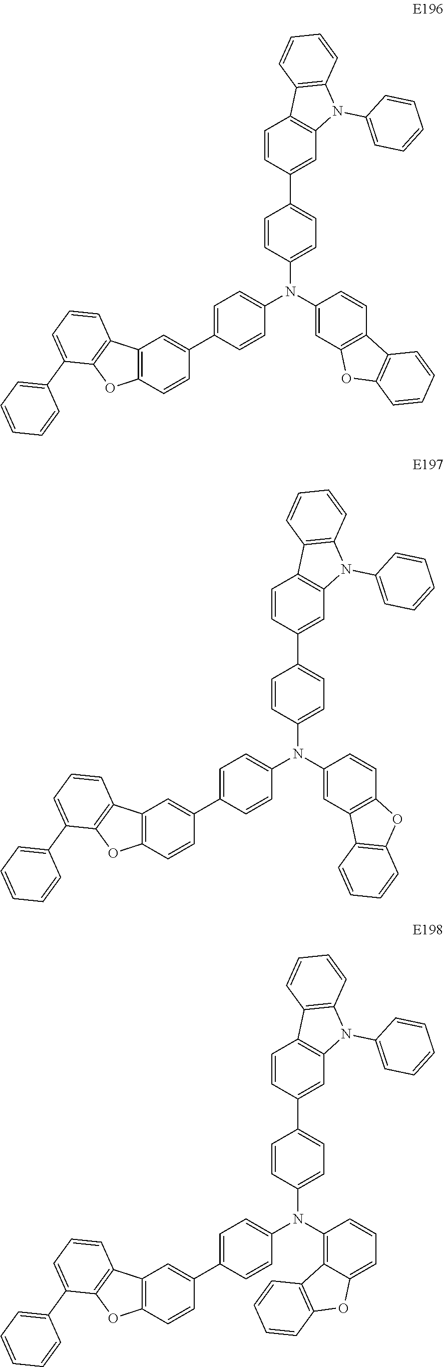 [SMILES: c1ccc(-c2cccc3c2oc2ccc(-c4ccc(N(c5ccc(-c6ccc7c8ccccc8n(-c8ccccc8)c7c6)cc5)c5ccc6c(c5)oc5ccccc56)cc4)cc23)cc1 ; c1ccc(-c2cccc3c2oc2ccc(-c4ccc(N(c5ccc(-c6ccc7c8ccccc8n(-c8ccccc8)c7c6)cc5)c5ccc6oc7ccccc7c6c5)cc4)cc23)cc1 ; c1ccc(-c2cccc3c2oc2ccc(-c4ccc(N(c5ccc(-c6ccc7c8ccccc8n(-c8ccccc8)c7c6)cc5)c5cccc6oc7ccccc7c56)cc4)cc23)cc1]

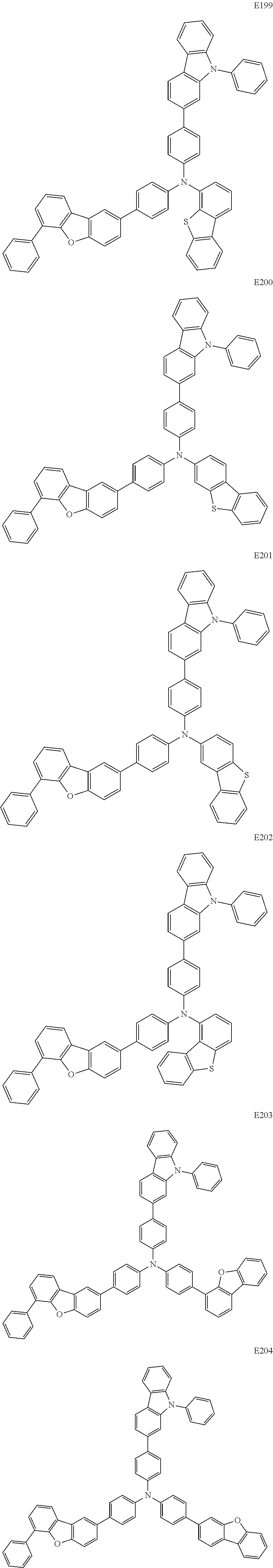

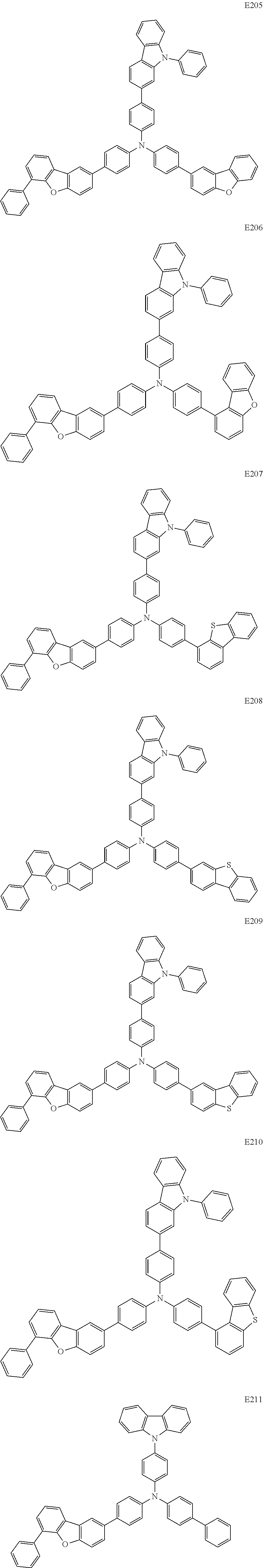

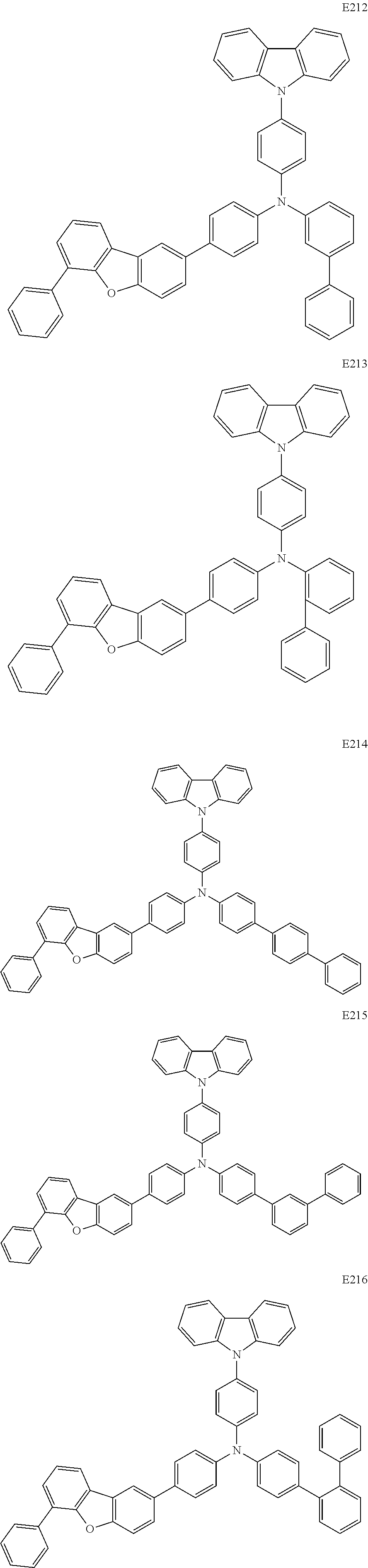

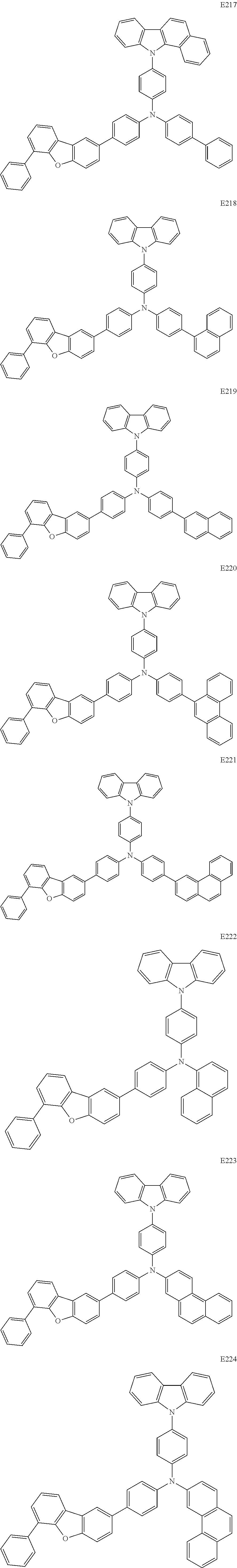

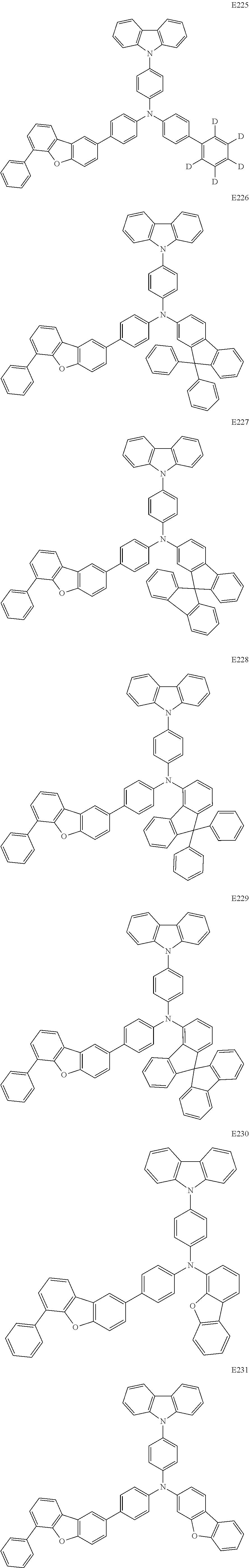

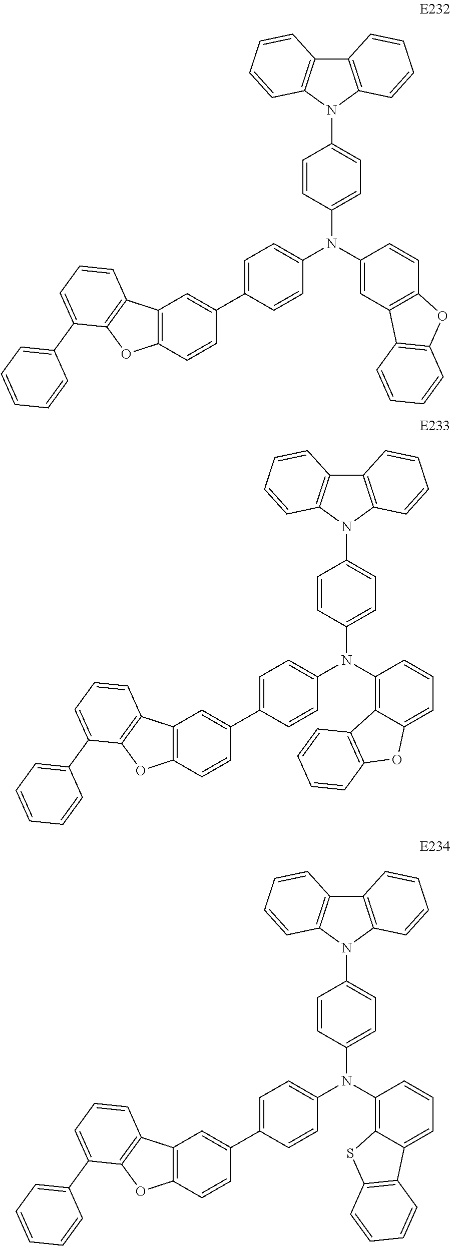 [SMILES: c1ccc(-c2cccc3c2oc2ccc(-c4ccc(N(c5ccc(-n6c7ccccc7c7ccccc76)cc5)c5ccc6oc7ccccc7c6c5)cc4)cc23)cc1 ; c1ccc(-c2cccc3c2oc2ccc(-c4ccc(N(c5ccc(-n6c7ccccc7c7ccccc76)cc5)c5cccc6oc7ccccc7c56)cc4)cc23)cc1 ; c1ccc(-c2cccc3c2oc2ccc(-c4ccc(N(c5ccc(-n6c7ccccc7c7ccccc76)cc5)c5cccc6c5sc5ccccc56)cc4)cc23)cc1]

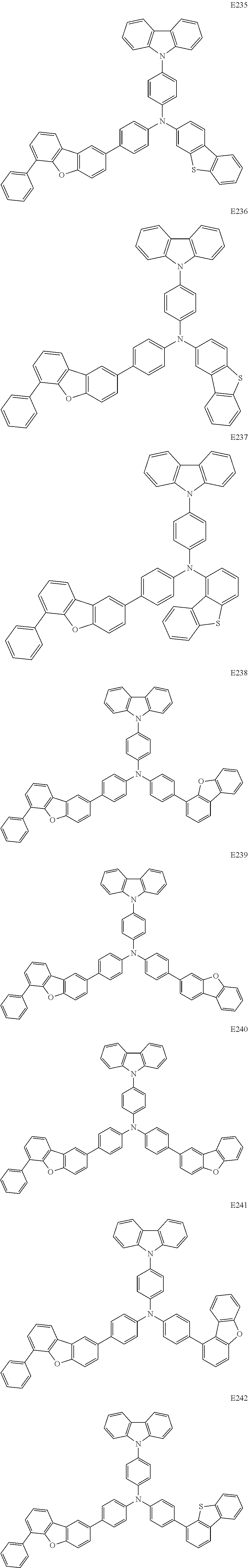

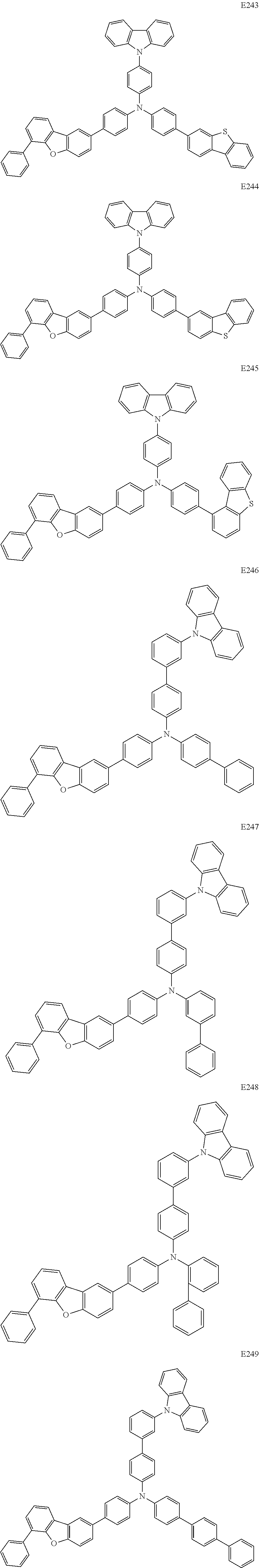

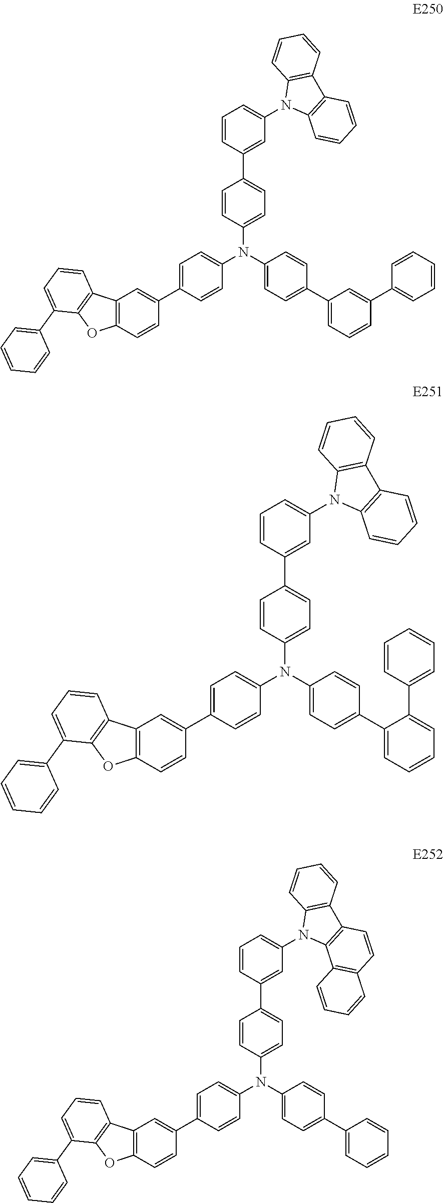 [SMILES: c1ccc(-c2cccc(-c3ccc(N(c4ccc(-c5cccc(-n6c7ccccc7c7ccccc76)c5)cc4)c4ccc(-c5ccc6oc7c(-c8ccccc8)cccc7c6c5)cc4)cc3)c2)cc1 ; c1ccc(-c2ccccc2-c2ccc(N(c3ccc(-c4cccc(-n5c6ccccc6c6ccccc65)c4)cc3)c3ccc(-c4ccc5oc6c(-c7ccccc7)cccc6c5c4)cc3)cc2)cc1 ; c1ccc(-c2ccc(N(c3ccc(-c4cccc(-n5c6ccccc6c6ccc7ccccc7c65)c4)cc3)c3ccc(-c4ccc5oc6c(-c7ccccc7)cccc6c5c4)cc3)cc2)cc1]

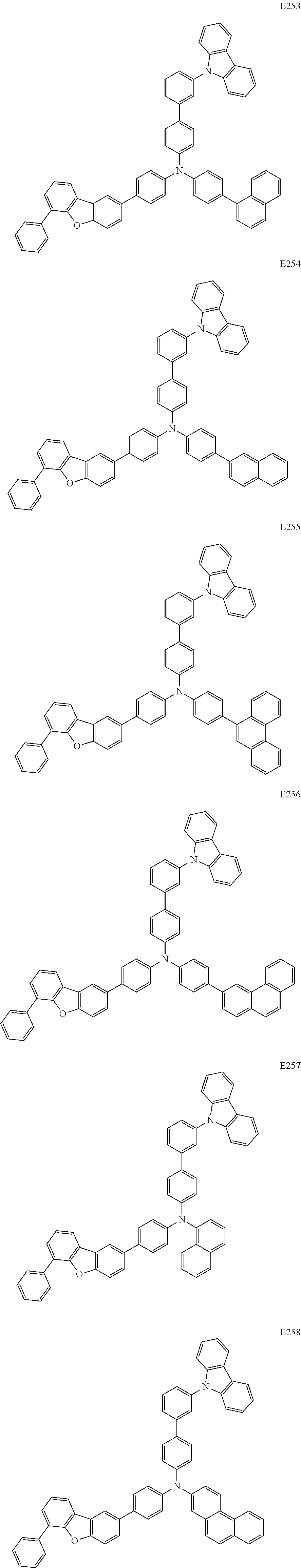

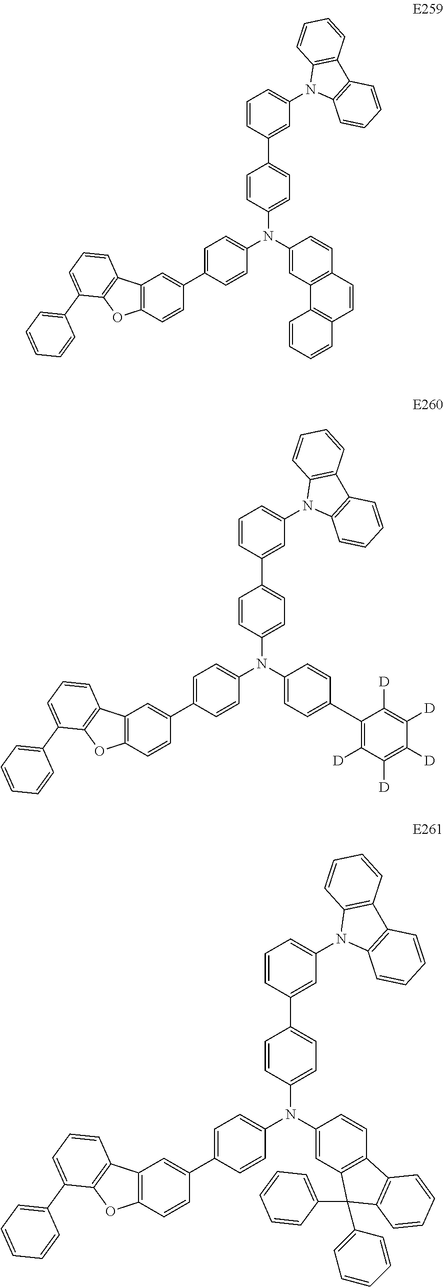 [SMILES: c1ccc(-c2cccc3c2oc2ccc(-c4ccc(N(c5ccc(-c6cccc(-n7c8ccccc8c8ccccc87)c6)cc5)c5ccc6ccc7ccccc7c6c5)cc4)cc23)cc1 ; [2H]c1c([2H])c([2H])c(-c2ccc(N(c3ccc(-c4cccc(-n5c6ccccc6c6ccccc65)c4)cc3)c3ccc(-c4ccc5oc6c(-c7ccccc7)cccc6c5c4)cc3)cc2)c([2H])c1[2H] ; c1ccc(-c2cccc3c2oc2ccc(-c4ccc(N(c5ccc(-c6cccc(-n7c8ccccc8c8ccccc87)c6)cc5)c5ccc6c(c5)C(c5ccccc5)(c5ccccc5)c5ccccc5-6)cc4)cc23)cc1]

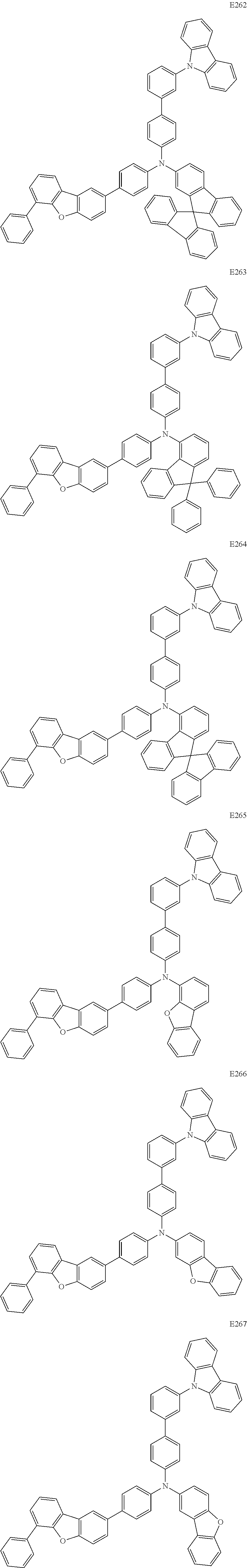

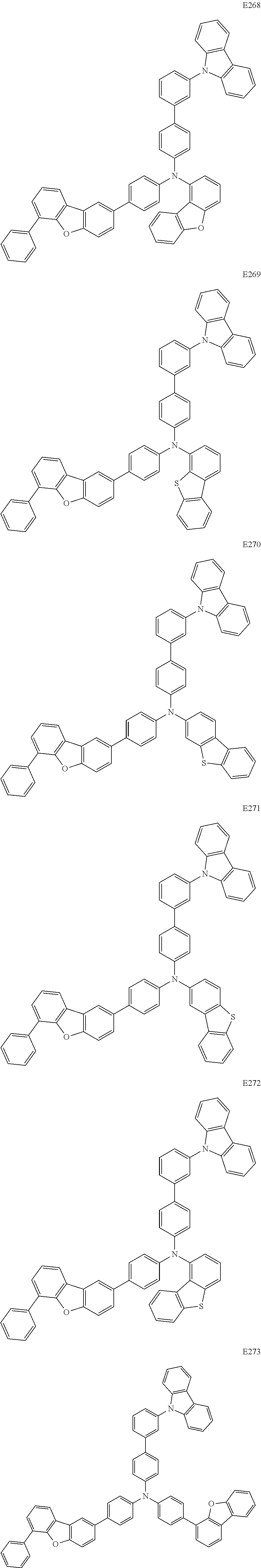

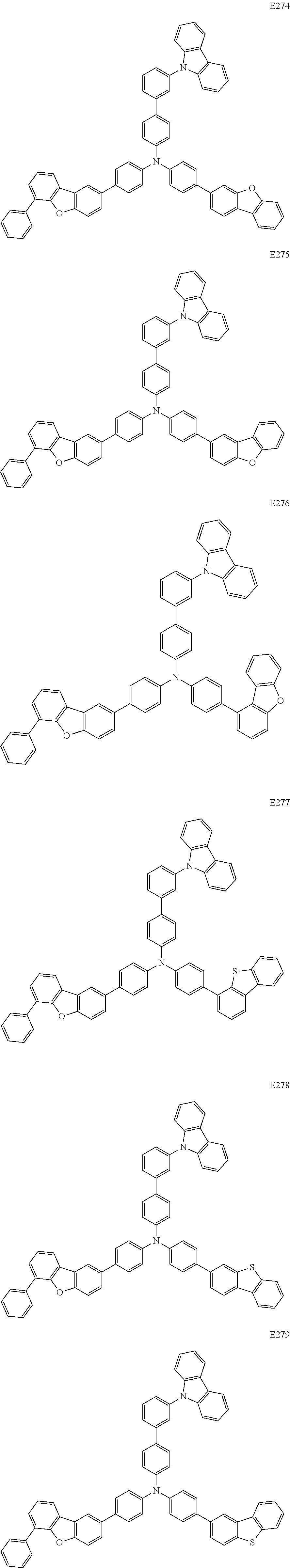

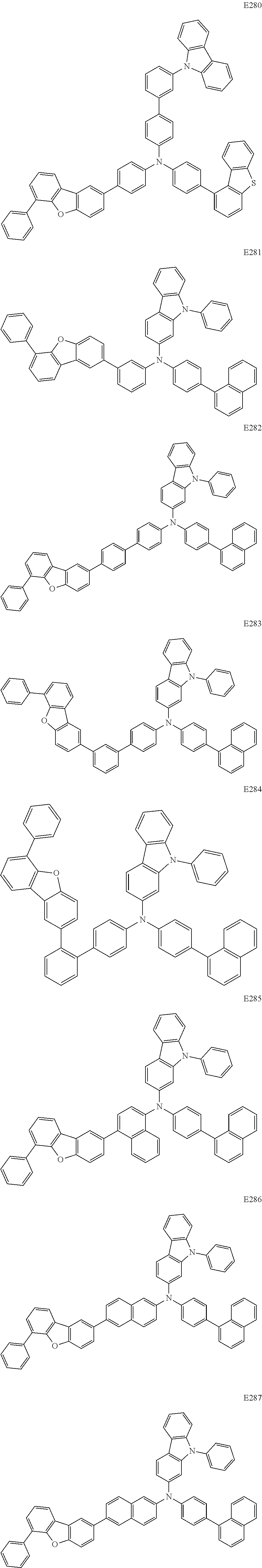

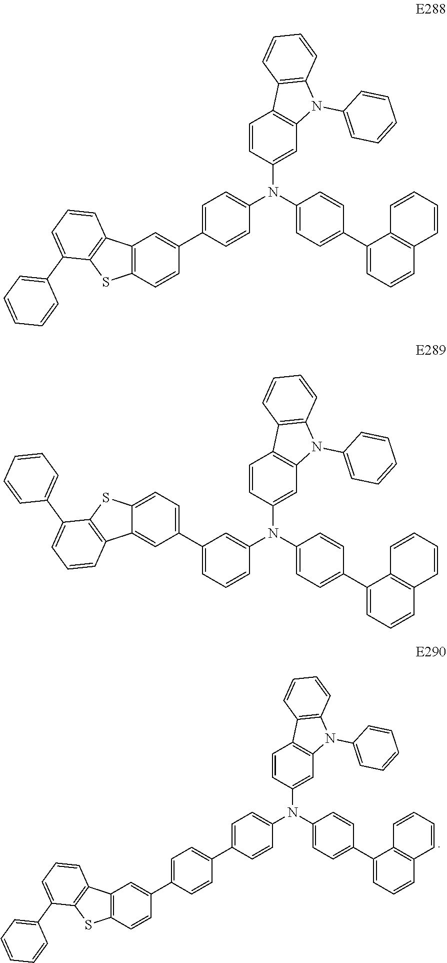 [SMILES: c1ccc(-c2cccc3c2sc2ccc(-c4ccc(N(c5ccc(-c6cccc7ccccc67)cc5)c5ccc6c7ccccc7n(-c7ccccc7)c6c5)cc4)cc23)cc1 ; c1ccc(-c2cccc3c2sc2ccc(-c4cccc(N(c5ccc(-c6cccc7ccccc67)cc5)c5ccc6c7ccccc7n(-c7ccccc7)c6c5)c4)cc23)cc1 ; c1ccc(-c2cccc3c2sc2ccc(-c4ccc(-c5ccc(N(c6ccc(-c7cccc8ccccc78)cc6)c6ccc7c8ccccc8n(-c8ccccc8)c7c6)cc5)cc4)cc23)cc1]

The amine compound represented by Formula 1 of an embodiment includes the dibenzoheterole group as a substituent, and for example, may have a feature in that carbon 2 of the dibenzoheterole group is bonded to the nitrogen atom of the amine group with a linker located therebetween. Accordingly, the amine compound of an embodiment may exhibit high stability in radical or radical cation state due to the expansion of the HOMO orbital. In some embodiments, the dibenzoheterole group included in the amine compound of may include a substituted or unsubstituted phenyl group substituted at carbon 6, thereby sterically protecting a heteroatom in the dibenzoheterole skeleton, and thus the stability of materials during the driving of the device may be improved. Moreover, the amine compound of an embodiment may include the first substituent as a substituent in addition to the dibenzoheterole group, and the introduction of a specific (suitable) polycyclic aromatic ring and heterocyclic ring may contribute to improving the electron resistance and exciton resistance of the material. The amine compound of an embodiment may have excellent electrical stability and high charge transport ability due to the introduction of a specific (suitable) substituent and the specification of the substitution position. In addition, the light emitting device of an embodiment including the amine compound of an embodiment may have an improvement in luminous efficiency and service life.

In some embodiments, the light emitting device ED may further include materials for the hole transport region, described further below, in the hole transport region HTR in addition to the above-described amine compound.

The hole transport region HTR may include a compound represented by Formula H-1:

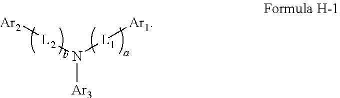

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer from 0 to 10. In some embodiments, when a or b is an integer of 2 or greater, a plurality of L₁s and L₂s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. Alternatively, the compound represented by Formula H-1 may be a diamine compound in which at least one among Ar₁ to Ar₃ includes the amine group as a substituent. In some embodiments, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one among the compounds of Compound Group H. However, the compounds listed in Compound Group H are merely examples, and the compounds represented by Formula H-1 are not limited to those represented by Compound Group H:

### Compound Group H

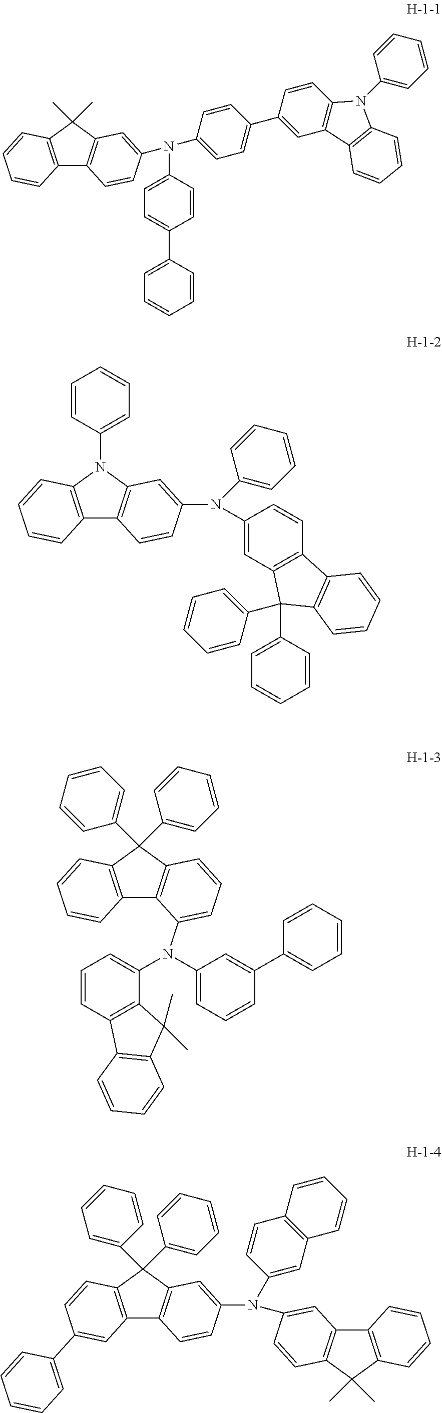 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

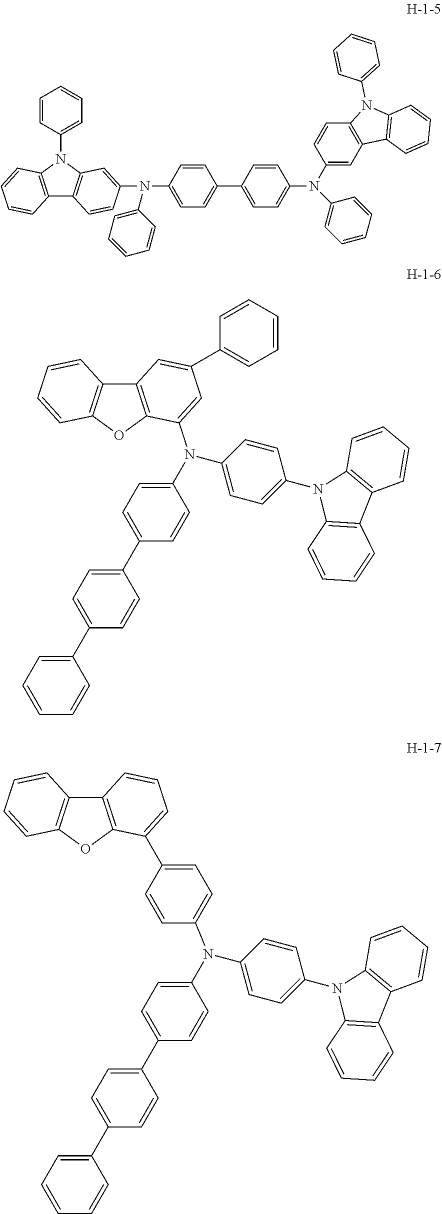 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

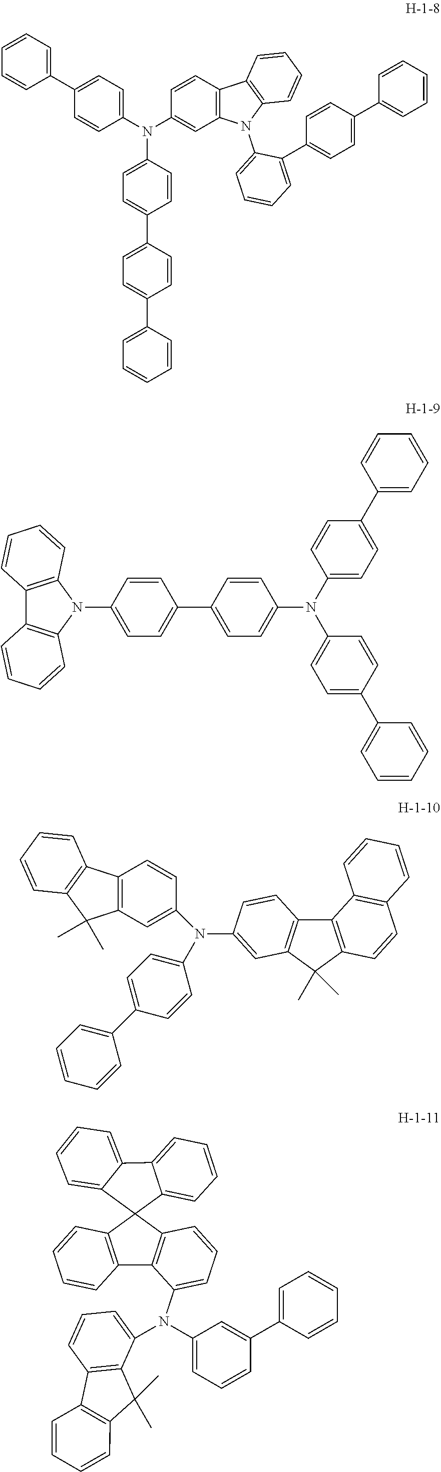

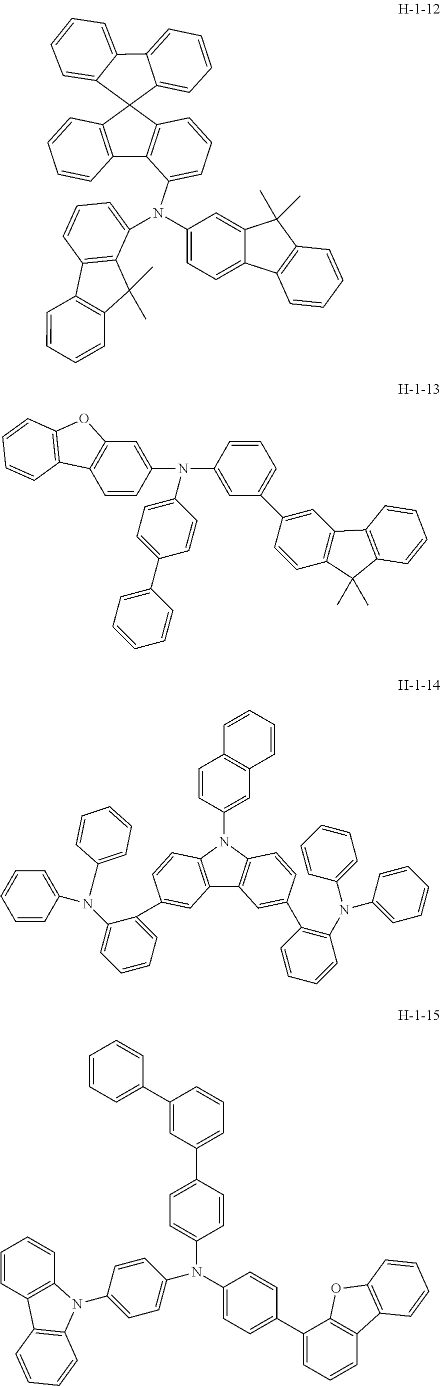 [SMILES: CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

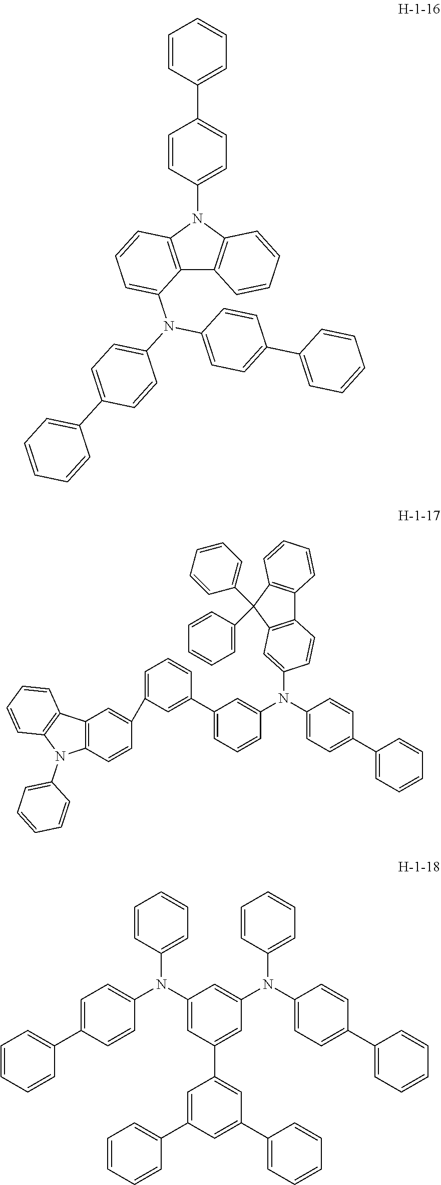 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

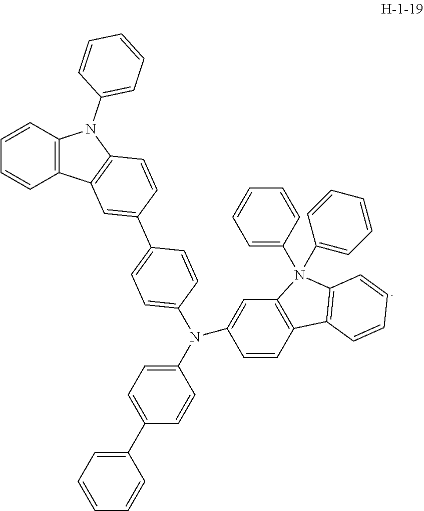

The hole transport region HTR may include a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N, N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-I-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-I-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the above-described ranges, satisfactory (suitable) hole transport properties may be achieved without a substantial increase in driving voltage.

The hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may include a metal halide compound such as CuI or RbI, a quinone derivative such as tetracyanoquinodimethane (TCNQ) or 2,3,5,6-tetrafluoro-7,7′8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cya nomethyl]-2,3,5,6-tetrafluorobenzonitrile (NDP9), etc., but the embodiment of the present disclosure is not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be contained in the hole transport region HTR may be used as a material to be contained in the buffer layer. The electron blocking layer EBL is a layer that serves to prevent (reduce) the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

In the light emitting device ED of an embodiment, the emission layer EML may include an anthracene derivative, a pyrene derivative, a fluoranthene derivative, a chrysene derivative, a dehydrobenzanthracene derivative, or a triphenylene derivative. For example, the emission layer EML may include the anthracene derivative or the pyrene derivative.

In each light emitting device ED of embodiments illustrated in FIGS. 3 to 6, the emission layer EML may include a host and a dopant, and the emission layer EML may include a compound represented by Formula E-1. The compound represented by Formula E-1 may be used as a fluorescent host material.

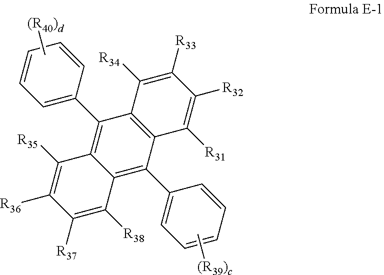

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer from 0 to 5.

Formula E-1 may be represented by any one among Compound E1 to Compound E19:

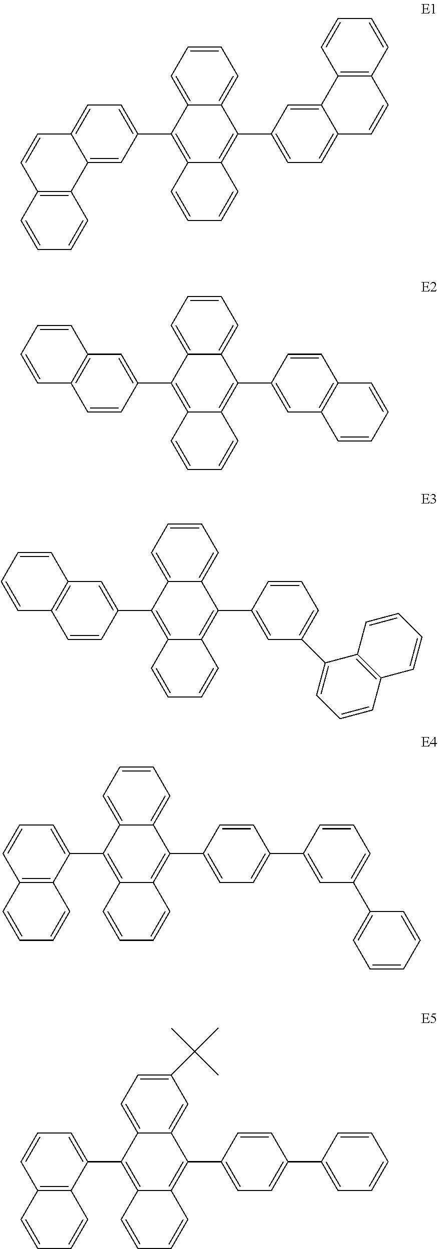

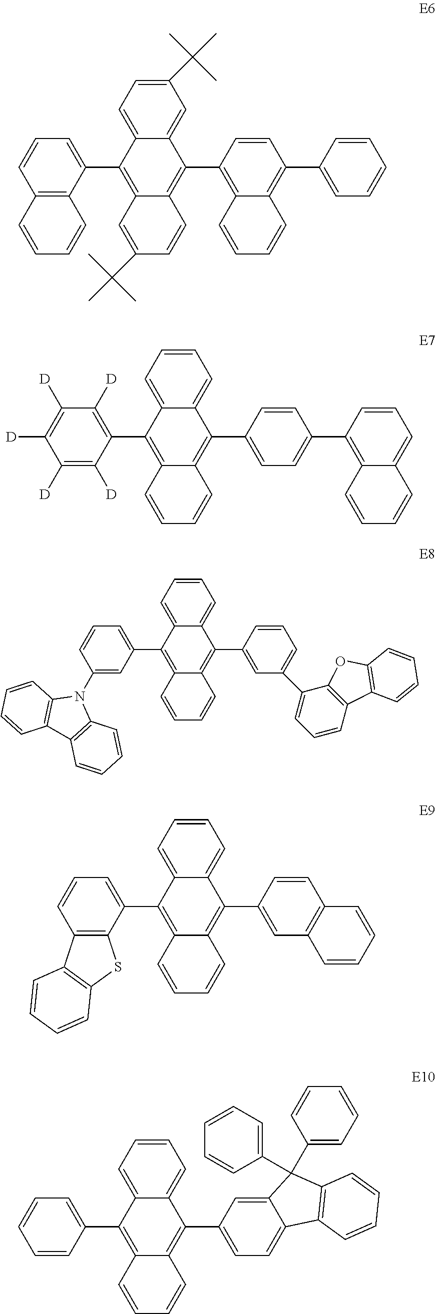

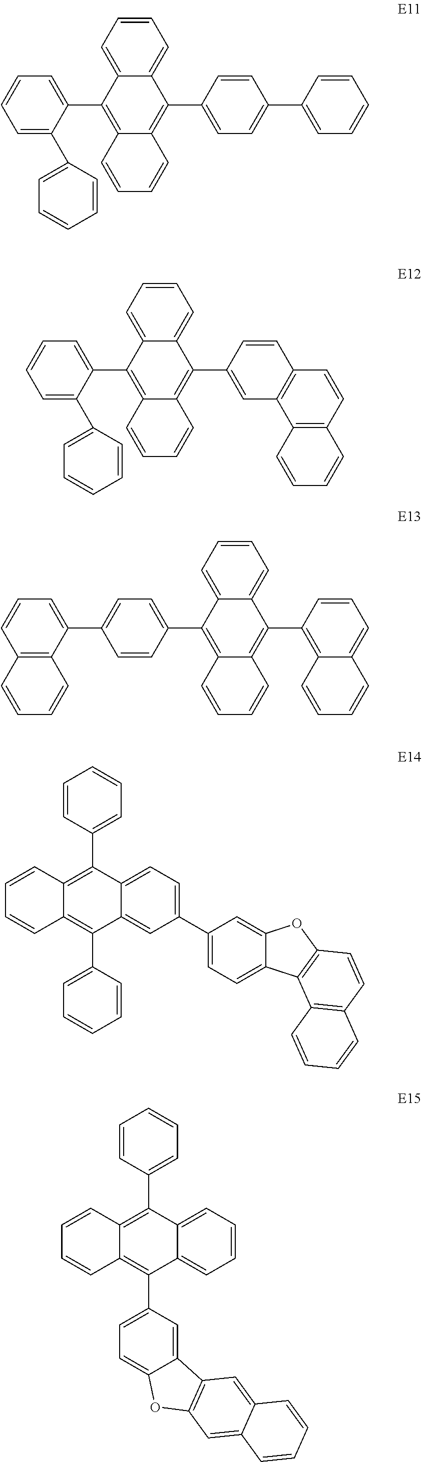

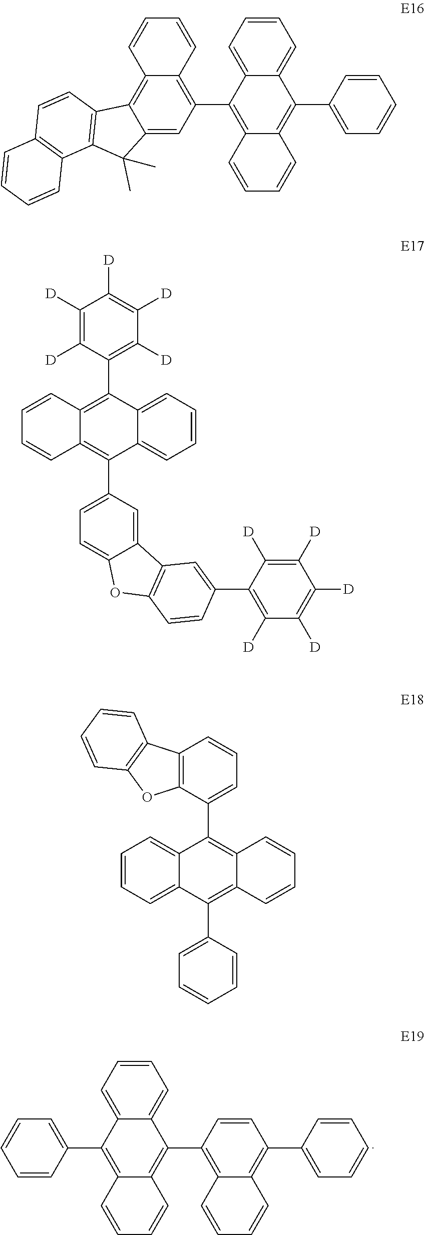

In an embodiment, the emission layer EML may include a compound represented by Formula E-2a or Formula E-2b. The compound represented by Formula E-2a or Formula E-2b may be used as a phosphorescent host material.

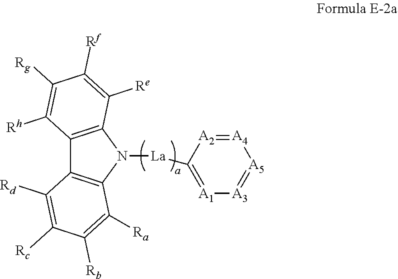

In Formula E-2a, a may be an integer from 0 to 10, La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or more, a plurality of Las may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, etc. as a ring-forming atom.

In some embodiments, in Formula E-2a, two or three selected from among A₁ to A₅ may be N, and the rest may be CRᵢ.

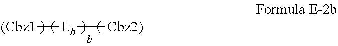

In Formula E-2b, Cbz1 and Cbz2 may each independently be an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer from 0 to 10, and when b is an integer of 2 or more, a plurality of L_{b}s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among the compounds of Compound Group E-2. However, the compounds listed in Compound Group E-2 are exemplary, and the compound represented by Formula E-2a or Formula E-2b is not limited to those represented in Compound Group E-2.

### Compound Group E-2

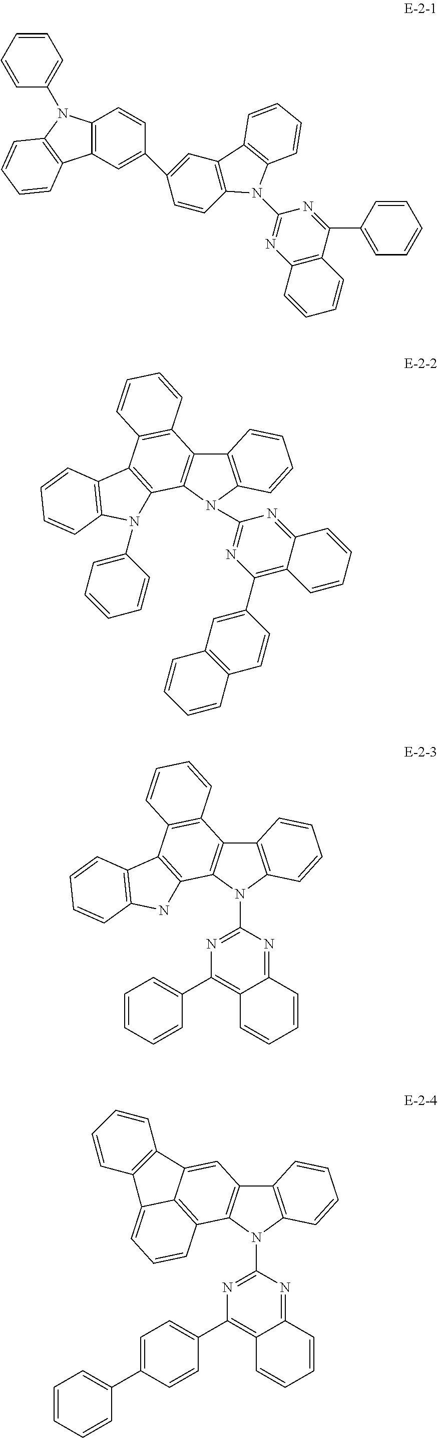

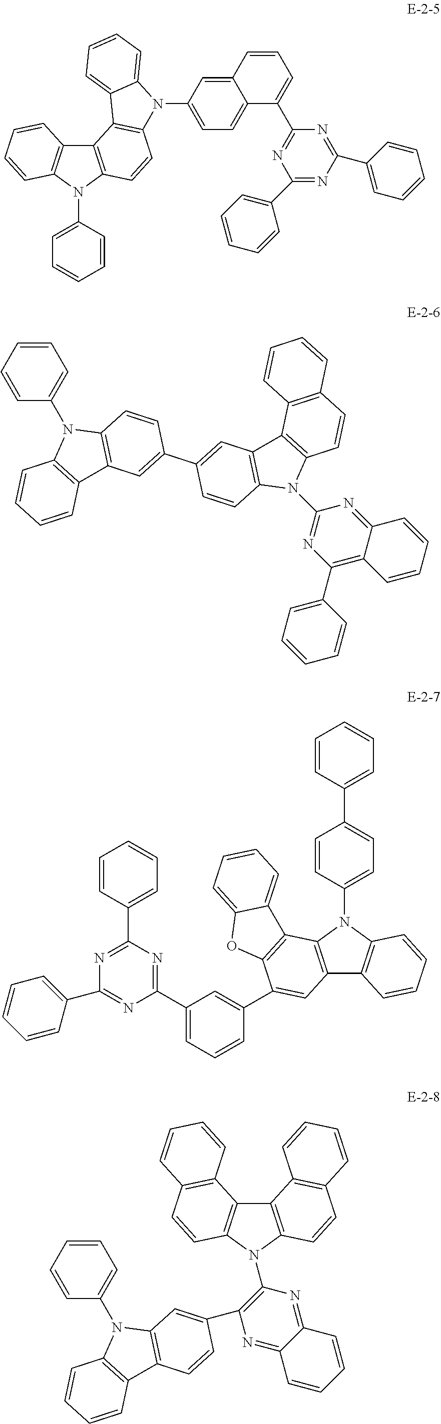 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

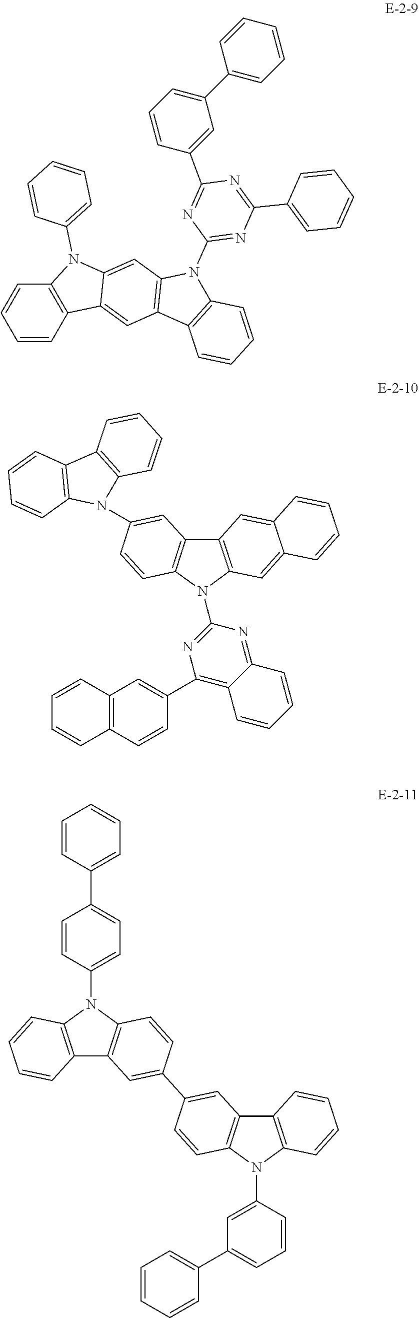 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

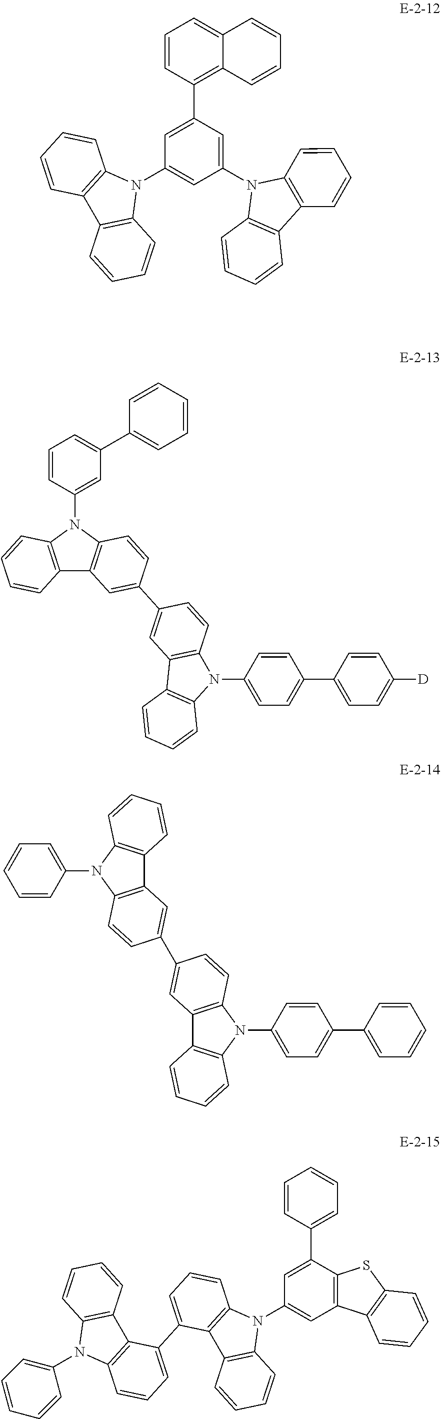 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

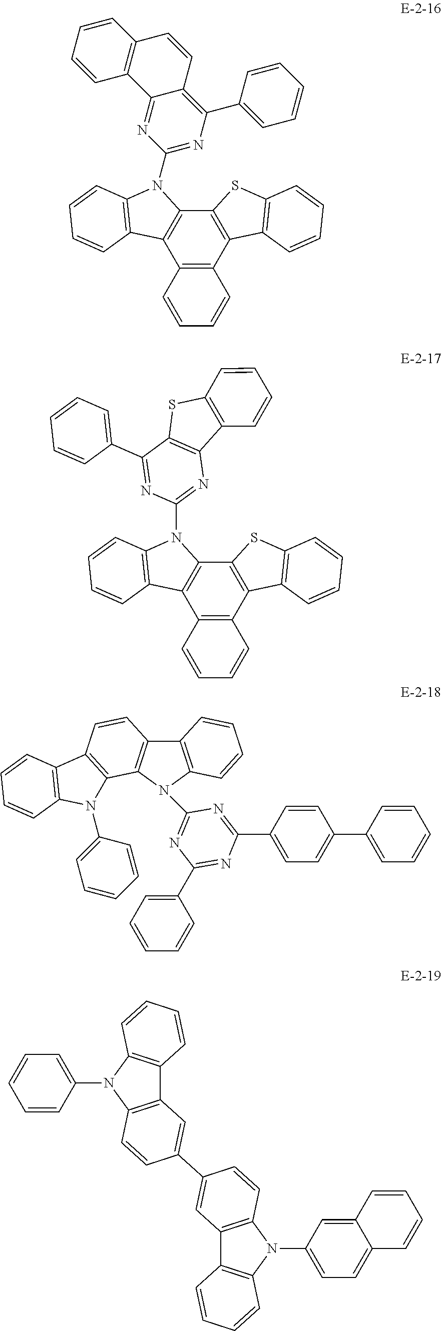

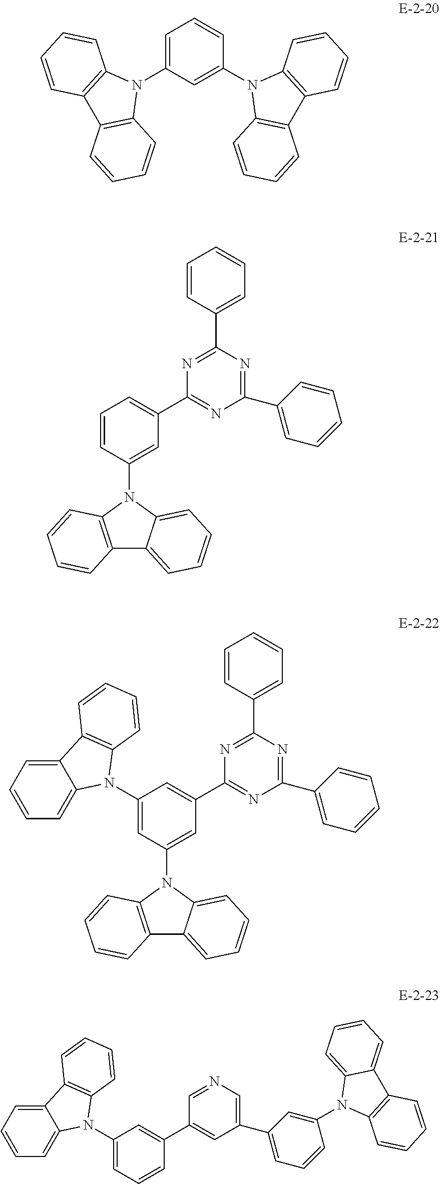 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

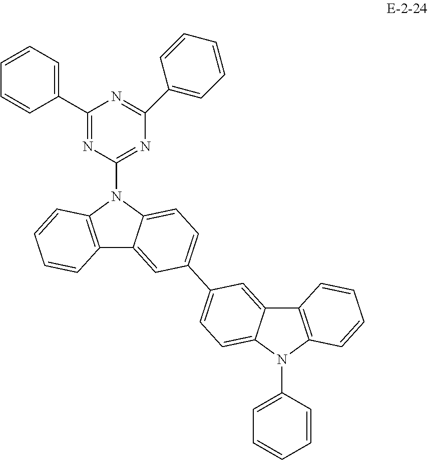 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material generally used in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the present disclosure is not limited thereto, for example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as a host material.

The emission layer EML may include a compound represented by Formula M-a or Formula M-b. The compound represented by Formula M-a or Formula M-b may be used as a phosphorescent dopant material.

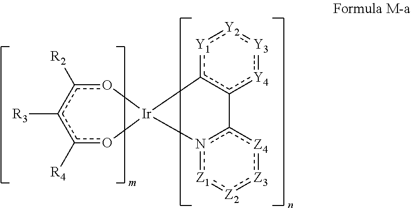

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m may be 0 or 1, and n may be 2 or 3. In Formula M-a, when m is 0, n is 3, and when m is 1, n is 2.

The compound represented by Formula M-a may be used as a phosphorescent dopant.

The compound represented by Formula M-a may be represented by any one among Compound M-a1 to Compound M-a25. However, Compounds M-a1 to M-a25 are merely examples, and the compound represented by Formula M-a is not limited to those represented by Compounds M-a1 to M-a25.

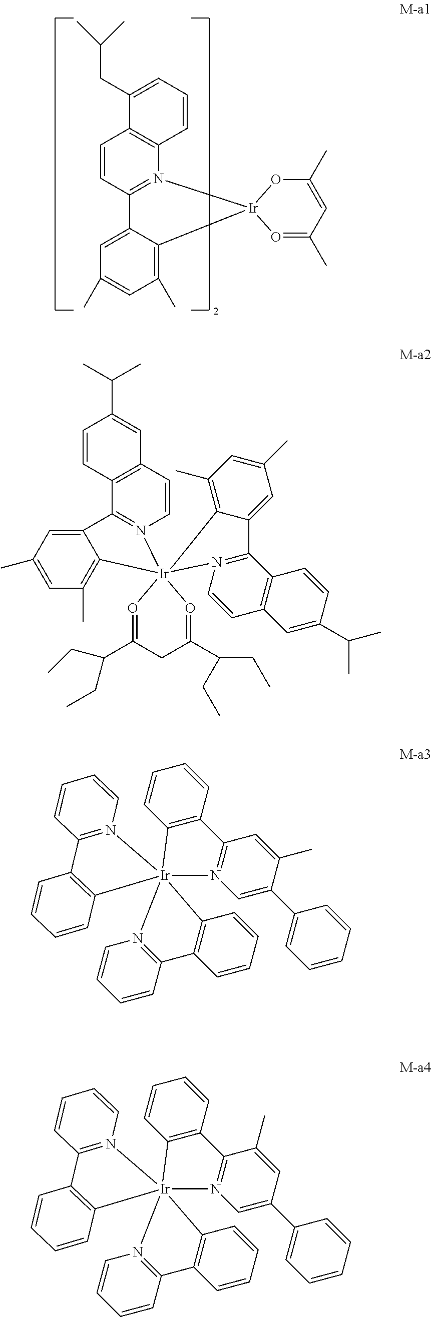

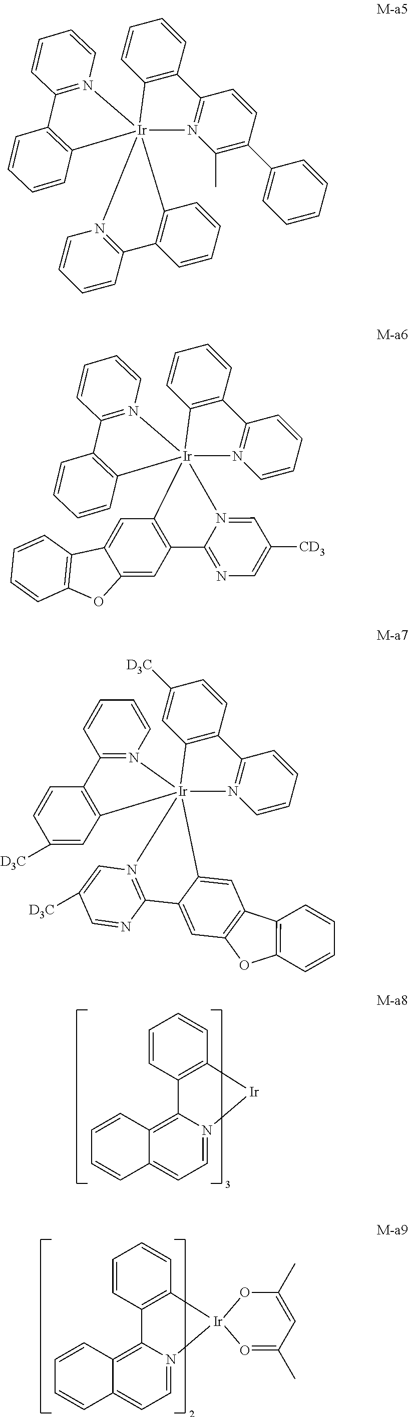

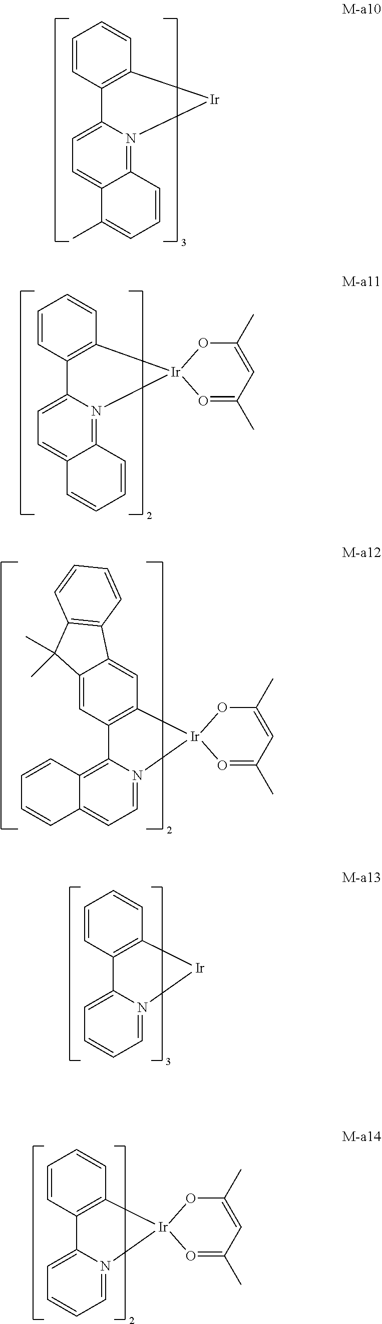

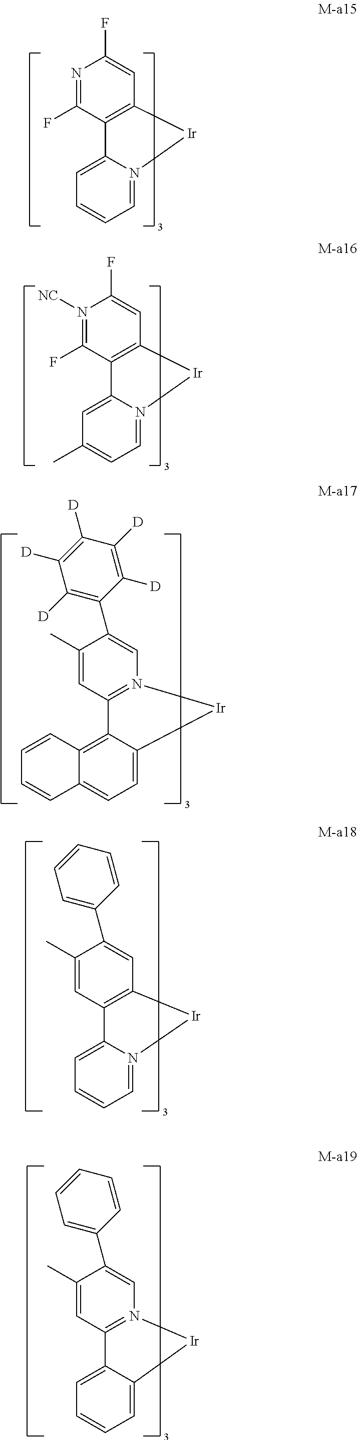

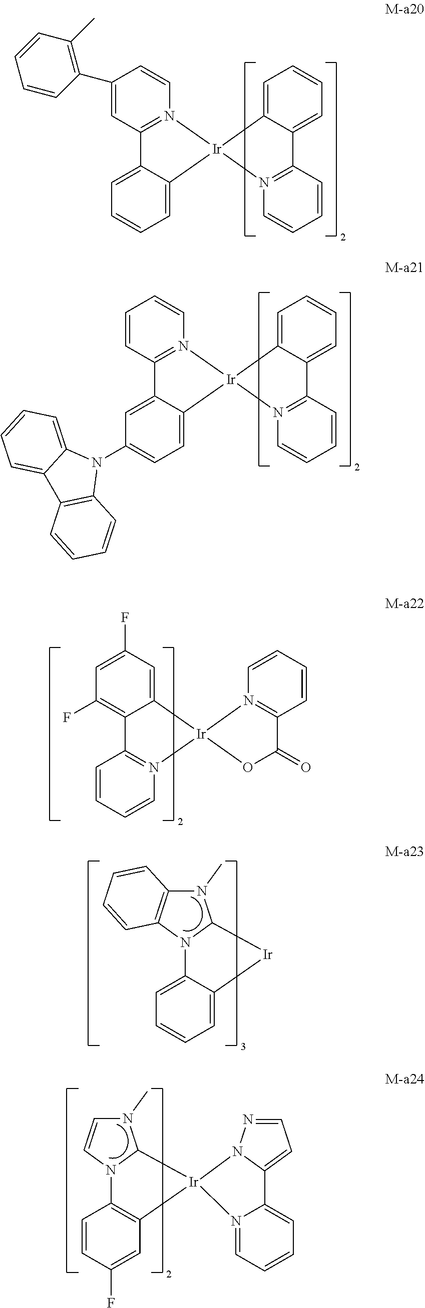

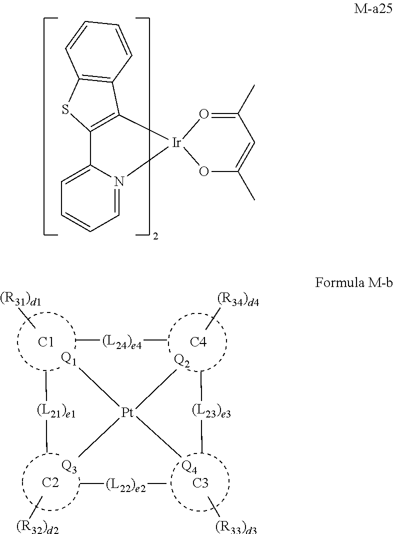

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently be a direct linkage,

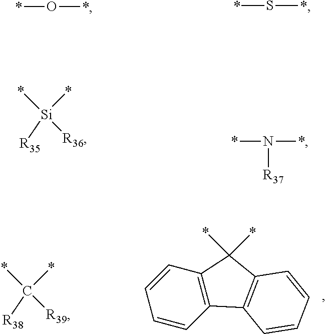

substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and d1 to d4 may each independently be an integer from 0 to 4.

The compound represented by Formula M-b may be used as a blue phosphorescent dopant or a green phosphorescent dopant.

The compound represented by Formula M-b may be represented by any one among the compounds below. However, the compounds below are merely examples, and the compound represented by Formula M-b is not limited to those represented by the compounds below.

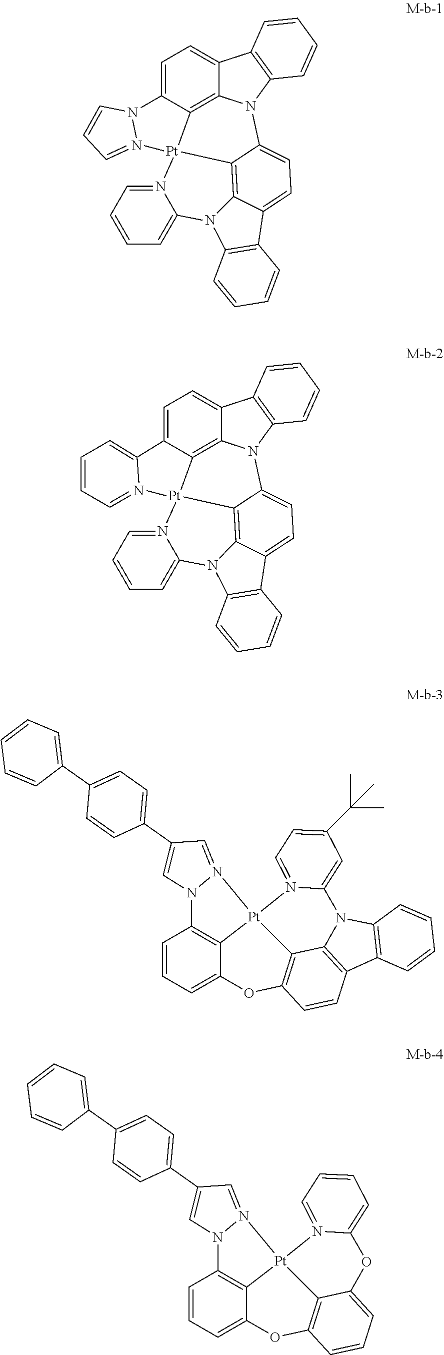

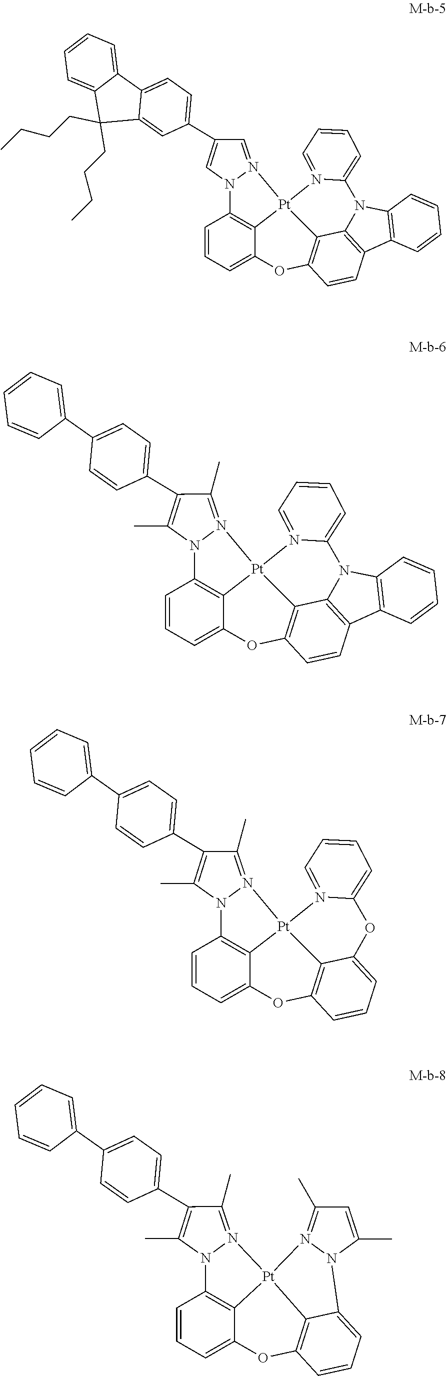

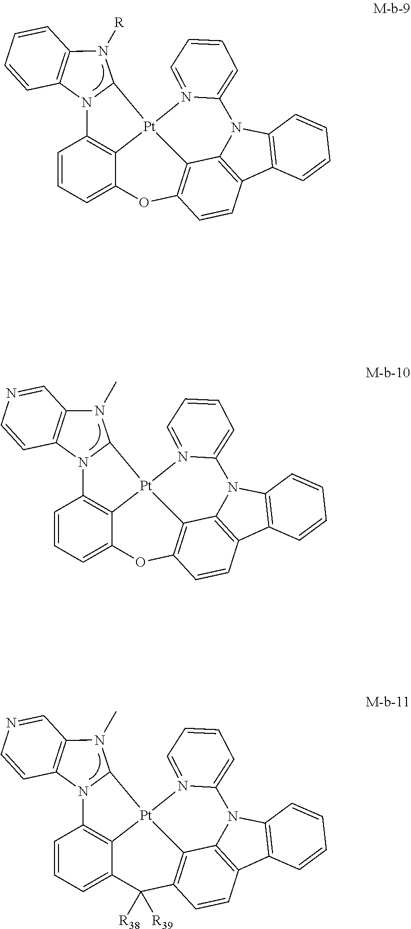

In the compounds, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one among Formula F-a to Formula F-c. The compound represented by Formula F-a or Formula F-c may be used as a fluorescence dopant material.

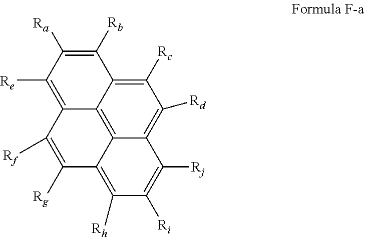

In Formula F-a, two selected from among Rₐ to Rⱼ may each independently be substituted with [symbol] NAr₁Ar₂. The others, which are not substituted with [symbol] NAr₁Ar₂, among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In [symbol] NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

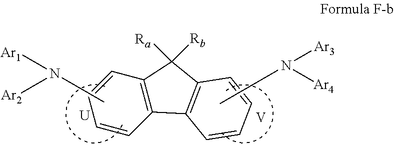

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one among Ar₁ to Ar₄ may be a heteroaryl group containing O or S as a ring-forming atom.

In Formula F-b, the number of rings represented by U and V may each independently be 0 or 1. For example, in Formula F-b, when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

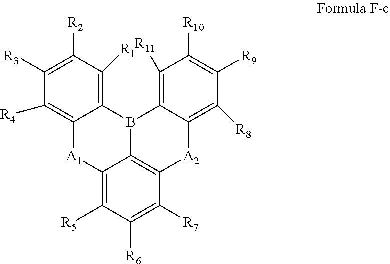

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a condensed ring. For example, when A₁ and A₂ are each independently NRₘ, A₁ may be bonded to R₄ or R₅ to form a ring. In some embodiments, A₂ may be bonded to R₇ or R₈ to form a ring.

In an embodiment, the emission layer EML may further include, as a generally used dopant material, styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenz enamine (N-BDAVBi), 4,4′-bis[2-(4-(N,N-diphenylamino)phenyl)vinyl]biphenyl (DPAVBi), perylene and/or the derivatives thereof (e.g., 2,5,8,11-tetra-t-butylperylene (TBP)), pyrene and/or the derivatives thereof (e.g., 1,1-dipyrene, 1,4-dipyrenylbenzene, 1,4-bis(N,N-diphenylamino)pyrene), etc.

The emission layer EML may further include a generally used phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), or platinum octaethyl porphyrin (PtOEP) may be used as a phosphorescent dopant. However, the embodiment of the present disclosure is not limited thereto.

The emission layer EML may include a quantum dot material. The core of the quantum dot may be selected from among a Group II-VI compound, a Group III-VI compound, a Group I-III-IV compound, a Group III-V compound, a Group III-II-V compound, a Group IV-VI compound, a Group IV element, a Group IV compound, and one or more combinations thereof.

The Group II-VI compound may be selected from the group including (e.g., consisting of) a binary compound selected from the group including (e.g., consisting of) CdSe, CdTe, CdS, ZnS, ZnSe, ZnTe, ZnO, HgS, HgSe, HgTe, MgSe, MgS, and a mixture thereof, a ternary compound selected from the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and one or more mixtures thereof, and a quaternary compound selected from the group including (e.g., consisting of) HgZnTeS, CdZnSeS, CdZnSeTe, CdZnSTe, CdHgSeS, CdHgSeTe, CdHgSTe, HgZnSeS, HgZnSeTe, HgZnSTe, and one or more mixtures thereof.

The Group III-VI compound may include a binary compound such as In₂S₃ or In₂Se₃, a ternary compound such as InGaS₃ or InGaSe₃, or one or more combinations thereof.

The Group I-III-VI compound may be selected from a ternary compound selected from the group including (e.g., consisting of) AgInS, AgInS₂, CuInS, CuInS₂, AgGaS₂, CuGaS₂ CuGaO₂, AgGaO₂, AgAlO₂, and a mixture thereof, or a quaternary compound such as AgInGaS₂ or CuInGaS₂.

The Group III-V compound may be selected from the group consisting of a binary compound selected from the group including (e.g., consisting of) GaN, GaP, GaAs, GaSb, AlN, AlP, AlAs, AlSb, InN, InP, InAs, InSb, and a mixture thereof, a ternary compound selected from the group including (e.g., consisting of) GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and one or more mixtures thereof, and a quaternary compound selected from the group including (e.g., consisting of) GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and one or more mixtures thereof. In some embodiments, the Group III-V compound may further include a Group II metal. For example, InZnP, etc. may be selected as a Group III-II-V compound.

The Group IV-VI compound may be selected from the group including (e.g., consisting of) a binary compound selected from the group including (e.g., consisting of) SnS, SnSe, SnTe, PbS, PbSe, PbTe, and a mixture thereof, a ternary compound selected from the group including (e.g., consisting of) SnSeS, SnSeTe, SnSTe, PbSeS, PbSeTe, PbSTe, SnPbS, SnPbSe, SnPbTe, and one or more mixtures thereof, and a quaternary compound selected from the group including (e.g., consisting of) SnPbSSe, SnPbSeTe, SnPbSTe, and one or more mixtures thereof. The Group IV element may be selected from the group including (e.g., consisting of) Si, Ge, and one or more mixtures thereof. The Group IV compound may be a binary compound selected from the group including (e.g., consisting of) SiC, SiGe, and one or more mixtures thereof.

In this embodiment, a binary compound, a ternary compound, or a quaternary compound may be present in a particle with a substantially uniform concentration distribution, or may be present in the same particle with a partially different concentration distribution. In some embodiments, a core/shell structure in which one quantum dot surrounds another quantum dot may also be possible. The core/shell structure may have a concentration gradient in which the concentration of elements present in the shell decreases toward the core.

In some embodiments, the quantum dot may have the above-described core/shell structure including a core containing nanocrystals and a shell surrounding the core. The shell of the quantum dot may serve as a protection layer to prevent (reduce) the chemical deformation of the core so as to maintain semiconductor properties, and/or a charging layer to impart electrophoresis properties to the quantum dot. The shell may be a single layer or a multilayer. An example of the shell of the quantum dot may include a metal or non-metal oxide, a semiconductor compound, or one or more combinations thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the embodiment of the present disclosure is not limited thereto.

Also, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have a full width of half maximum (FWHM) of a light emitting wavelength spectrum of about 45 nm or less, about 40 nm or less, and about 30 nm or less, and color purity or color reproducibility may be improved in the above range. In some embodiments, light emitted through such a quantum dot is emitted in all directions, and thus a viewing angle may be improved (increased).

In some embodiments, although the form of a quantum dot is not limited as long as it is a form generally used in the art, for example, a quantum dot in the form of substantially spherical, pyramidal, multi-arm, or cubic nanoparticles, nanotubes, nanowires, nanofibers, nanoparticles, etc. may be used.

The quantum dot may control the color of emitted light according to the particle size thereof, and accordingly, the quantum dot may have one or more suitable emission colors such as blue, red, and green.

In each light emitting device ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiment of the present disclosure is not limited thereto. The electron transport region ETR may have a thickness, for example, from about 1,000 Å to about 1,500 Å.

The electron transport region ETR may be formed by using one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1:

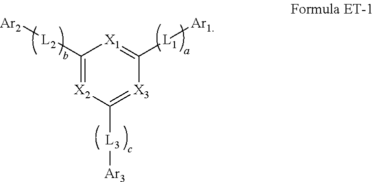

In Formula ET-1, at least one among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), berylliumbis(benzoquinolin-10-olate) (Bebq₂), 9,10-di(naphthalene-2-yl)anthracene) (ADN), 1,3-bis[3,5-di(pyridin-3-yl)phenyl]benzene (BmPyPhB), or one or more mixtures thereof.

The electron transport region ETR may include at least one among Compound ET1 to Compound ET36:

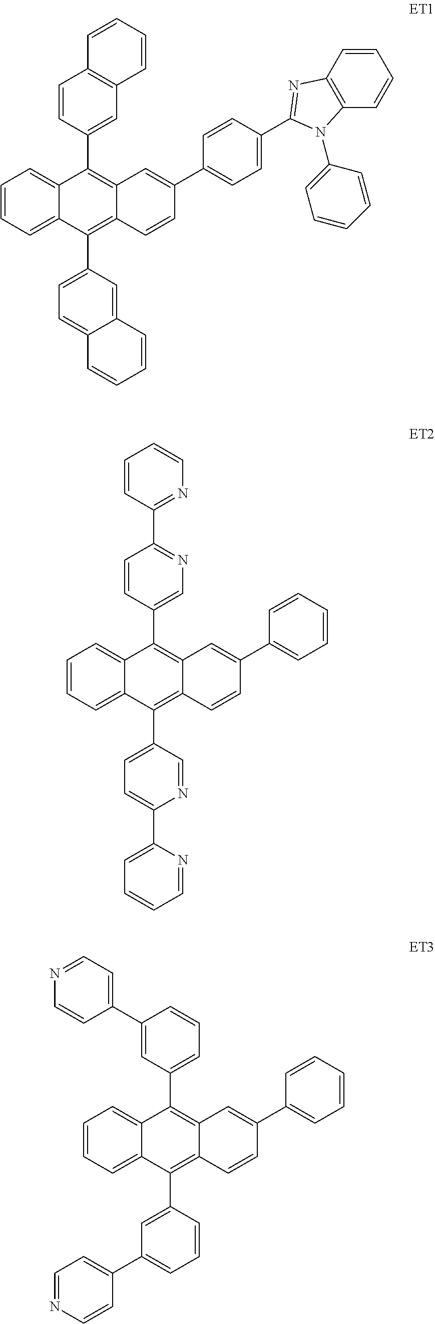 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

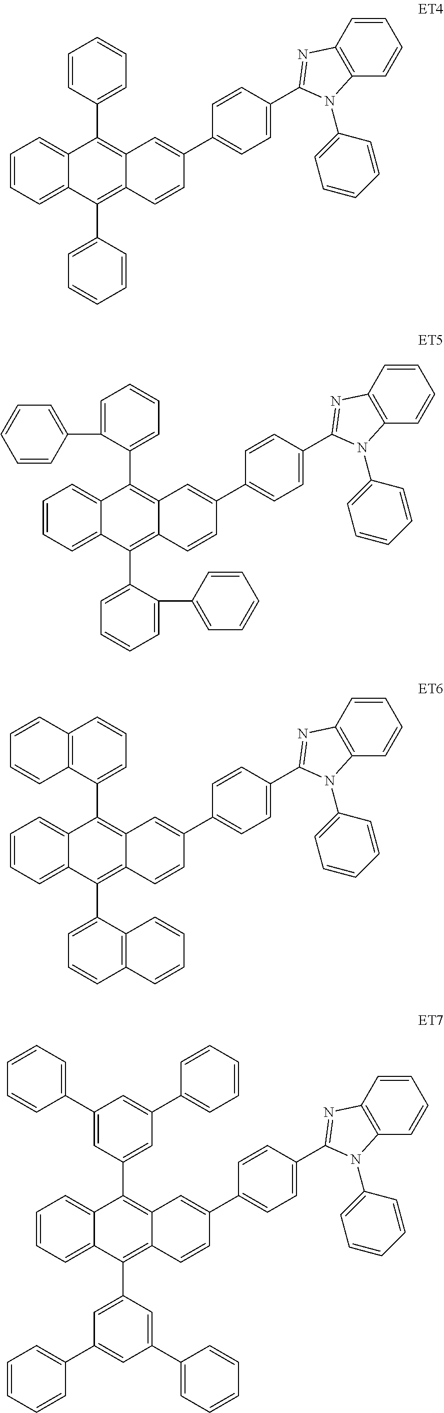 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

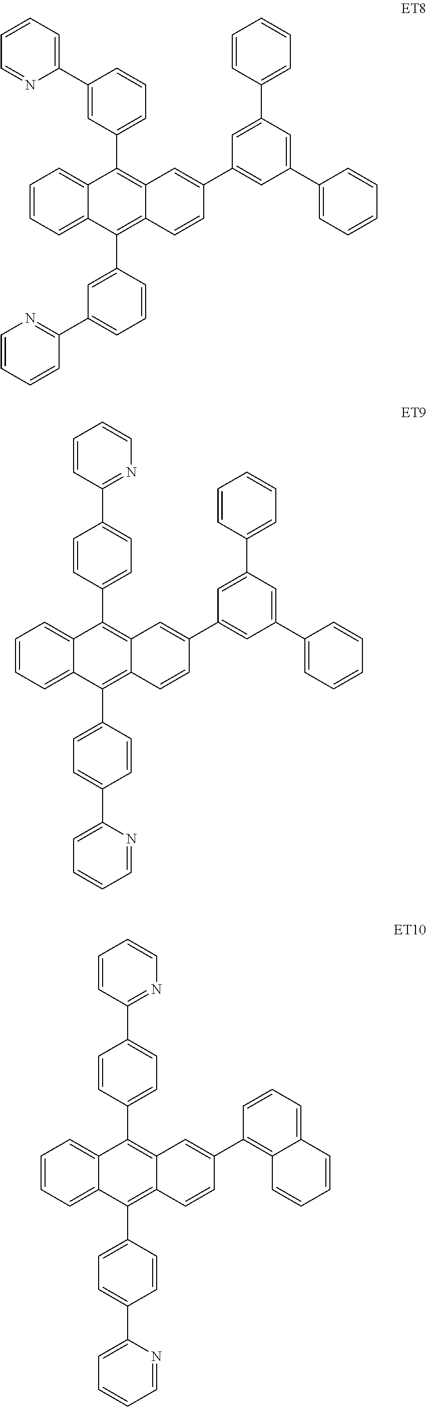 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

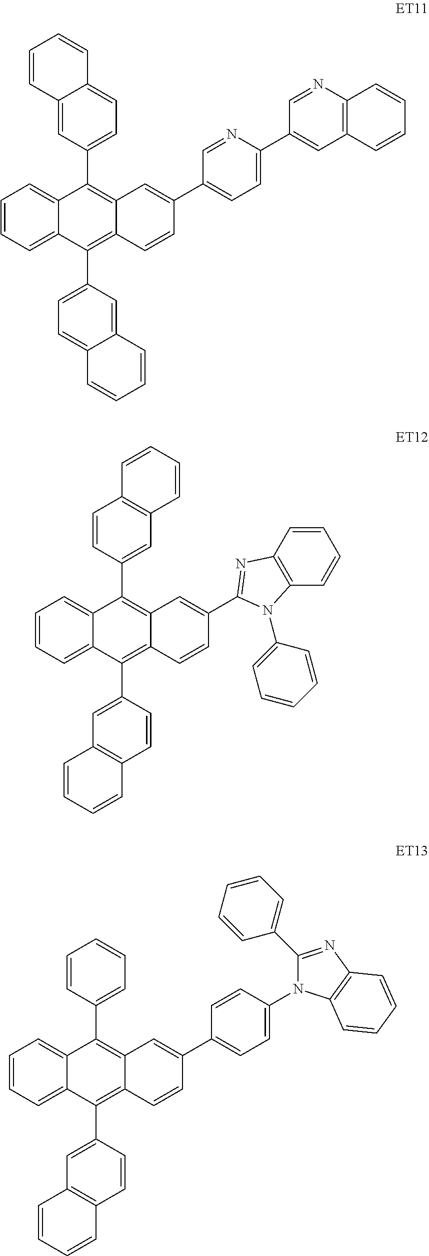 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

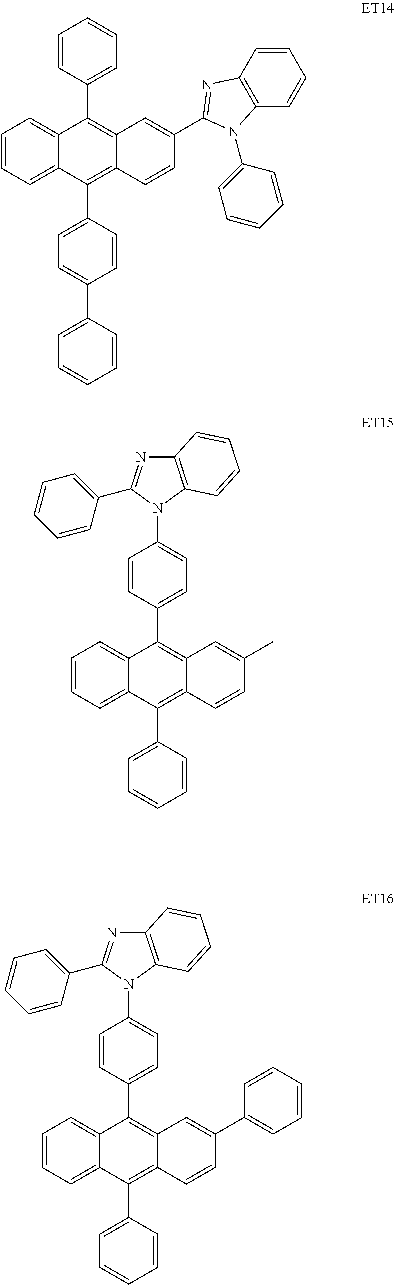 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

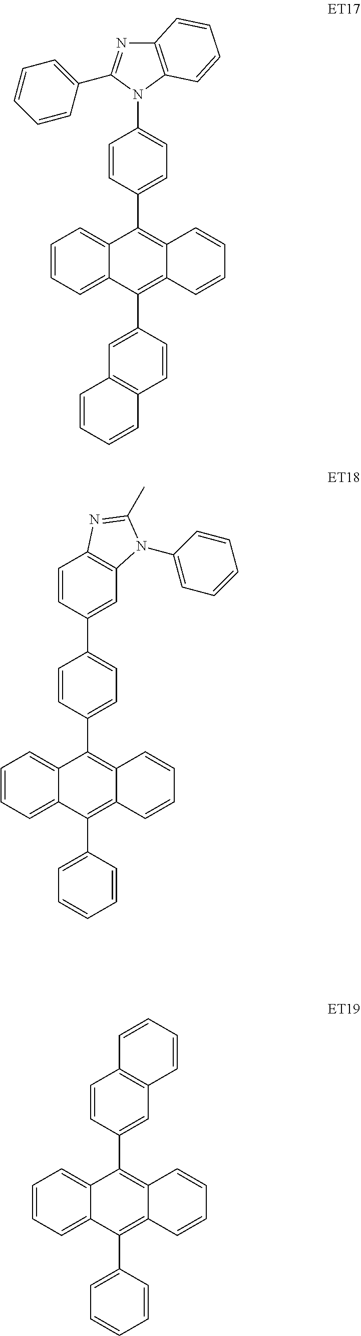

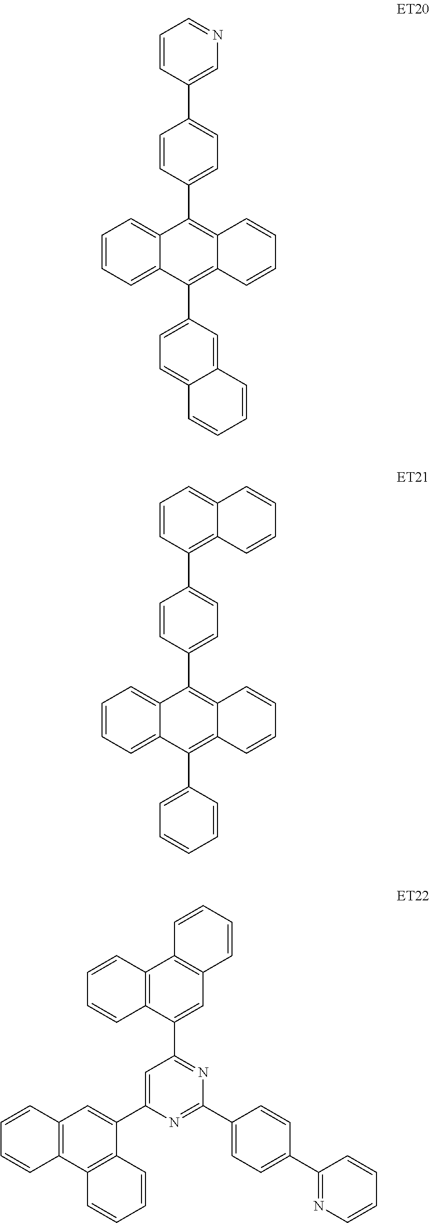

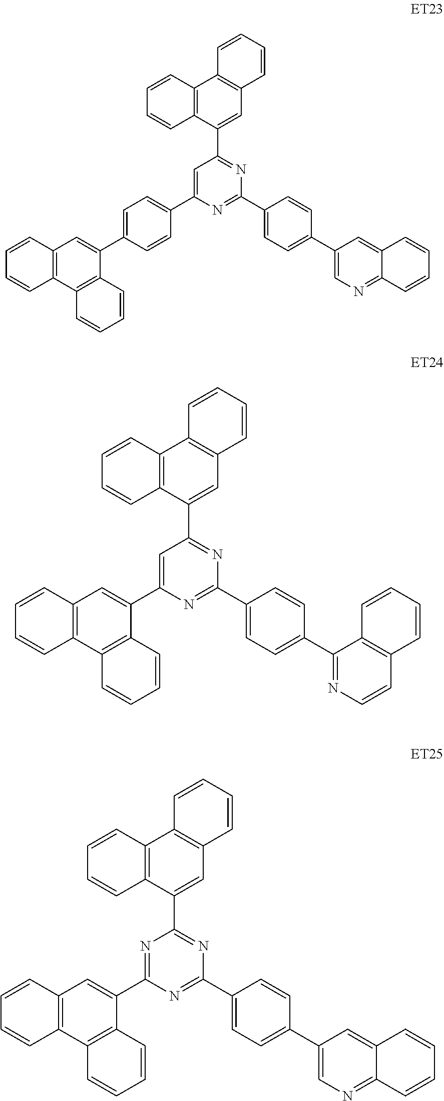 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

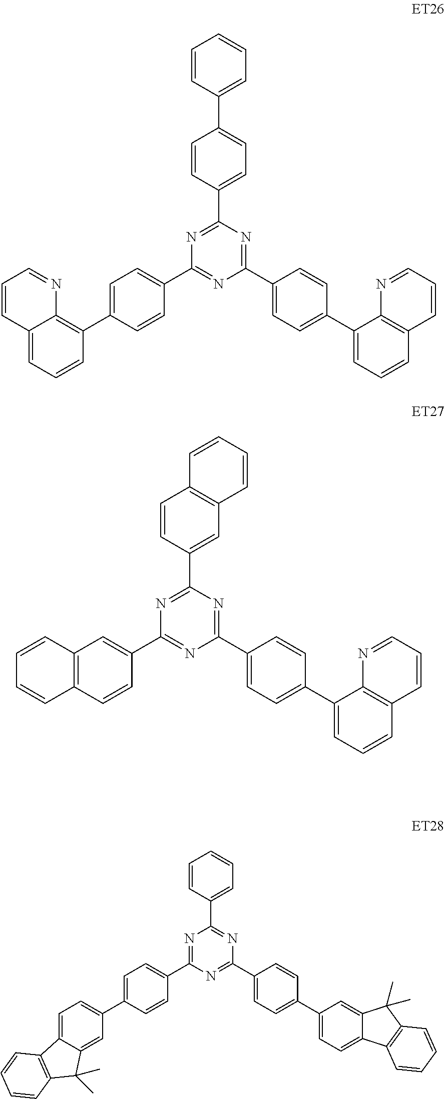 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

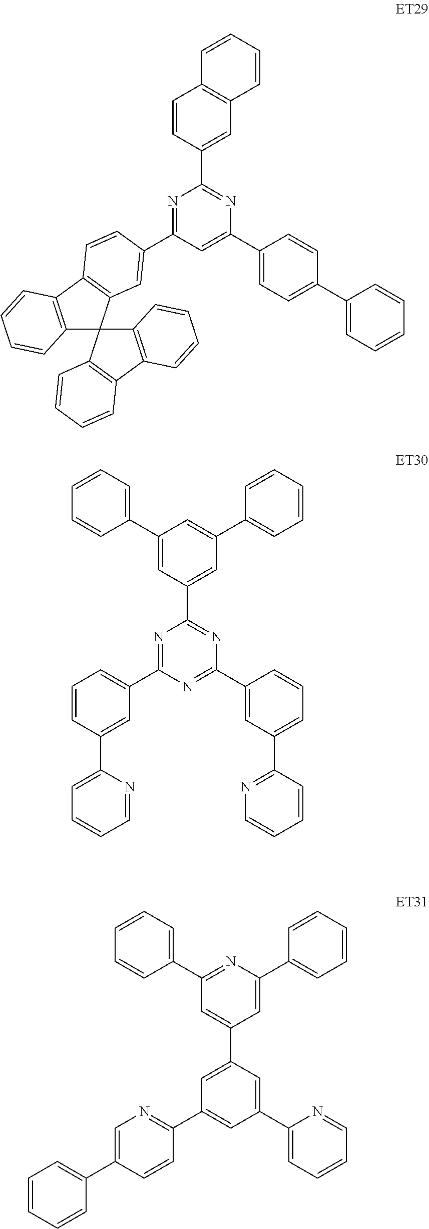

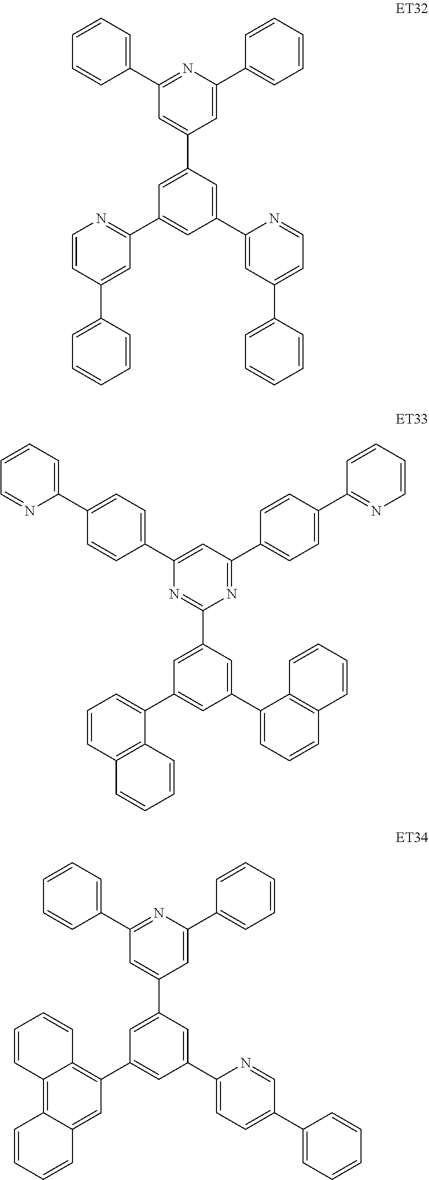 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

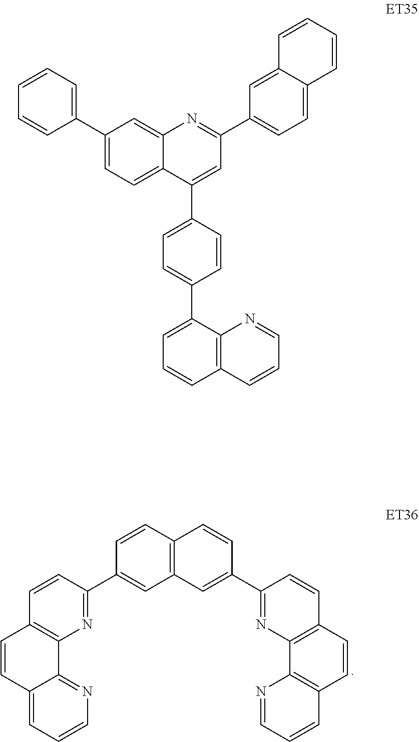 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; "."]

In some embodiments, the electron transport regions ETR may include a metal halide such as LiF, NaCl, CsF, RbCl, RbI, CuI, and/or KI, a lanthanide metal such as Yb, and a co-deposited material of the metal halide and the lanthanide metal. For example, the electron transport region ETR may include KI:Yb, RbI:Yb, LiF:Yb, etc. as a co-deposited material. In some embodiments, the electron transport region ETR may be formed using a metal oxide such as Li₂O or BaO, or 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. The organometallic salt may include, for example, a metal acetate, a metal benzoate, a metal acetoacetate, a metal acetylacetonate, and/or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but the embodiment of the present disclosure is not limited thereto.

The electron transport region ETR may include the above-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, satisfactory (suitable) electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the above-described range, satisfactory (suitable) electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or one or more compounds or mixtures thereof (e.g., AgMg, AgYb, or MgAg). Alternatively, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

The second electrode EL2 may be connected with an auxiliary electrode. When the second electrode EL2 is connected with the auxiliary electrode, the resistance of the second electrode EL2 may be decreased.

In some embodiments, a capping layer CPL may further be disposed on the second electrode EL2 of the light emitting device ED of an embodiment. The capping layer CPL may include a multilayer or a single layer.

In an embodiment, the capping layer CPL may be an organic layer or an inorganic layer. For example, when the capping layer CPL contains an inorganic material, the inorganic material may include an alkaline metal compound (for example, LiF), an alkaline earth metal compound (for example, MgF₂), SiON, SiNx, SiOy, etc.

For example, when the capping layer CPL contains an organic material, the organic material may include 2,2′-dimethyl-N,N′-di-[(1-naphthyl)-N, N′-diphenyl]-1,1′-biphenyl-4,4′-diamine (α-NPD), NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or may include an epoxy resin, or an acrylate such as a methacrylate. However, the embodiment of the present disclosure is not limited thereto, and the capping layer CPL may include at least one among Compounds P1 to P5:

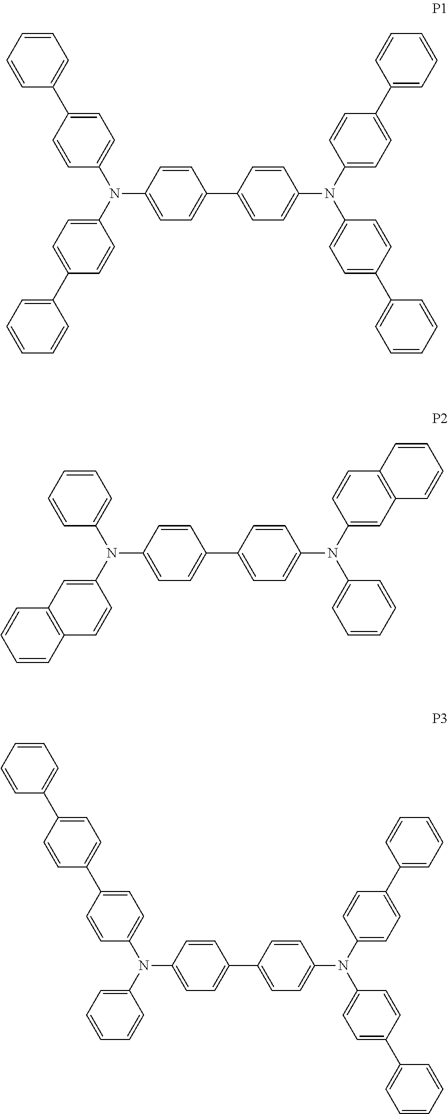 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

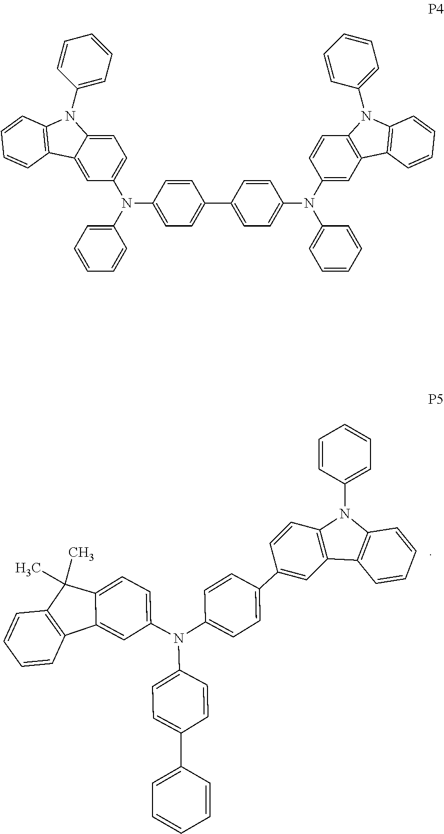 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

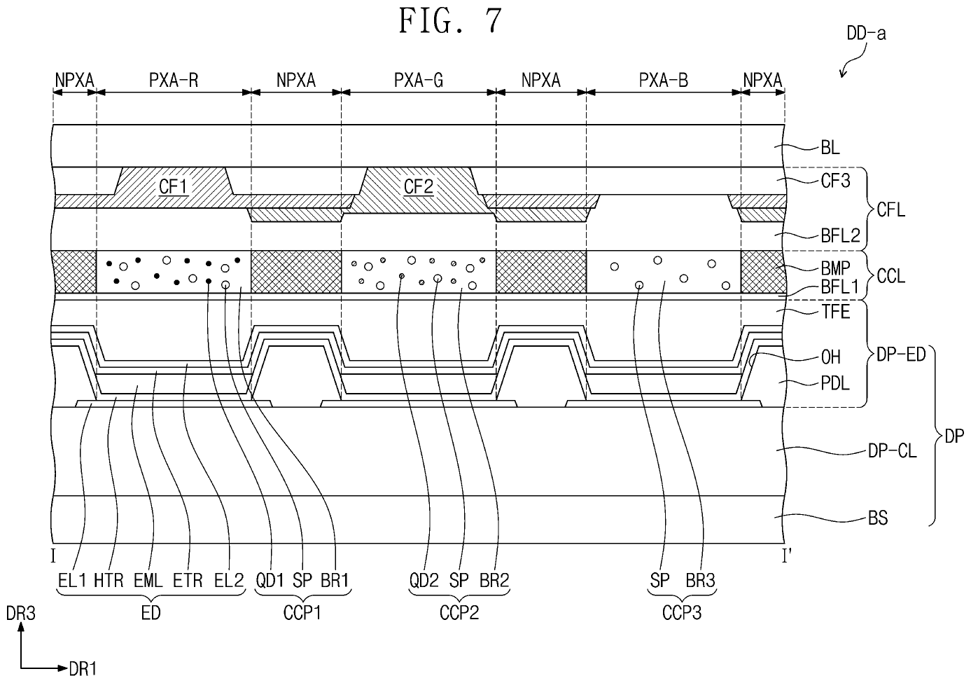

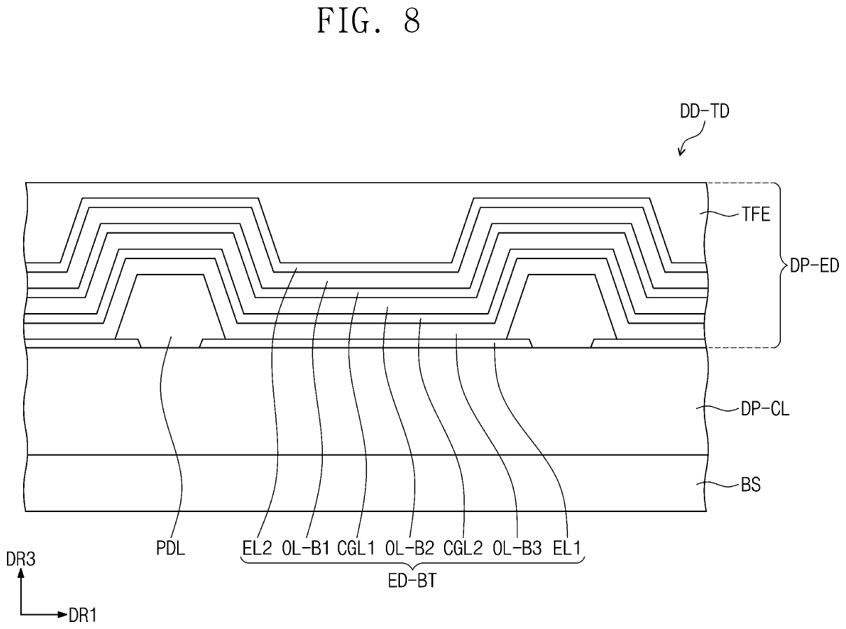

FIGS. 7 and 8 each are a cross-sectional view of a display apparatus according to an embodiment. Hereinafter, in describing the display apparatuses of embodiments with reference to FIGS. 7 and 8, the duplicated features which have been described in FIGS. 1 to 6 may not be described again, but their differences will primarily be described.

Referring to FIG. 7, the display apparatus DD according to an embodiment may include a display panel DP including a display device layer DP-ED, a light control layer CCL on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In some embodiments, the structures of the light emitting devices of FIGS. 3 to 6 as described above may be equally applied to the structure of the light emitting device ED illustrated in FIG. 7.

Referring to FIG. 7, the emission layer EML may be in an opening OH defined in a pixel defining film PDL. For example, the emission layer EML which is divided by the pixel defining film PDL and provided corresponding to each light emitting regions PXA-R, PXA-G, and PXA-B may emit light in substantially the same wavelength range. In the display apparatus DD of an embodiment, the emission layer EML may emit blue light. In some embodiments, the emission layer EML may be provided as a common layer in the entire light emitting regions PXA-R, PXA-G, and PXA-B.

The light control layer CCL may be on the display panel DP. The light control layer CCL may include a light conversion body. The light conversion body may be a quantum dot, a phosphor, and/or the like. The light conversion body may emit provided light by converting the wavelength thereof. For example, the light control layer CCL may be a layer containing the quantum dot or a layer containing the phosphor.

The light control layer CCL may include a plurality of light control parts CCP1, CCP2 and CCP3. The light control parts CCP1, CCP2, and CCP3 may be spaced apart from (separated from) each other.

Referring to FIG. 7, divided patterns BMP may be between the light control parts CCP1, CCP2 and CCP3 which are spaced apart from each other, but the embodiment of the present disclosure is not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting device ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting device ED. For example, the first quantum dot QD1 may be a red quantum dot, and the second quantum dot QD2 may be a green quantum dot. The same as described above may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may serve to prevent (reduce) the penetration of moisture and/or oxygen (hereinafter, referred to as ‘moisture/oxygen’). The barrier layer BFL1 may be on the light control parts CCP1, CCP2, and CCP3 to block (reduce) the light control parts CCP1, CCP2 and CCP3 from being exposed to moisture/oxygen. In some embodiments, the barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display apparatus DD of an embodiment, the color filter layer CFL may be on the light control layer CCL. For example, the color filter layer CFL may be directly on the light control layer CCL. In this embodiment, the barrier layer BFL2 may not be provided.

The color filter layer CFL may include a light shielding part BM and color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye. In some embodiments, the embodiment of the present disclosure is not limited thereto, and the third filter CF3 may not include (e.g., may exclude) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

In some embodiments, the first filter CF1 and the second filter CF2 may be a yellow filter. The first filter CF1 and the second filter CF2 may not be separated but be provided as one filter.

The light shielding part BM may be a black matrix. The light shielding part BM may include an organic light shielding material or an inorganic light shielding material containing a black pigment or dye. The light shielding part BM may prevent (reduce) light leakage, and may separate boundaries between the adjacent filters CF1, CF2, and CF3. In some embodiments, the light shielding part BM may be formed of a blue filter.

The first to third filters CF1, CF2, and CF3 may be disposed corresponding to the red light emitting region PXA-R, the green light emitting region PXA-G, and the blue light emitting region PXA-B, respectively.

A base substrate BL may be on the color filter layer CFL. The base substrate BL may be a member which provides a base surface in which the color filter layer CFL, the light control layer CCL, and/or the like are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided.

FIG. 8 is a cross-sectional view illustrating a portion of a display apparatus according to an embodiment of the present disclosure. FIG. 8 illustrates a cross-sectional view of a part corresponding to the display panel DP of FIG. 7. In the display apparatus DD-TD of an embodiment, the light emitting device ED-BT may include a plurality of light emitting structures OL-B1, OL-B2, and OL-B3. The light emitting device ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) therebetween.

For example, the light emitting device ED-BT included in the display apparatus DD-TD of an embodiment may be a light emitting device having a tandem structure and including a plurality of emission layers.

In an embodiment illustrated in FIG. 8, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting device ED-BT including the plurality of light emitting structures OL-B1, OL-B2, and OL-B3 which emit light beams having wavelength ranges different from each other may emit white light.

Charge generation layers CGL1 and CGL2 may be respectively disposed between two of the neighboring light emitting structures OL-B1, OL-B2, and OL-B3. The charge generation layers CGL1 and CGL2 may include a p-type (kind) charge generation layer and/or an n-type (kind) charge generation layer.

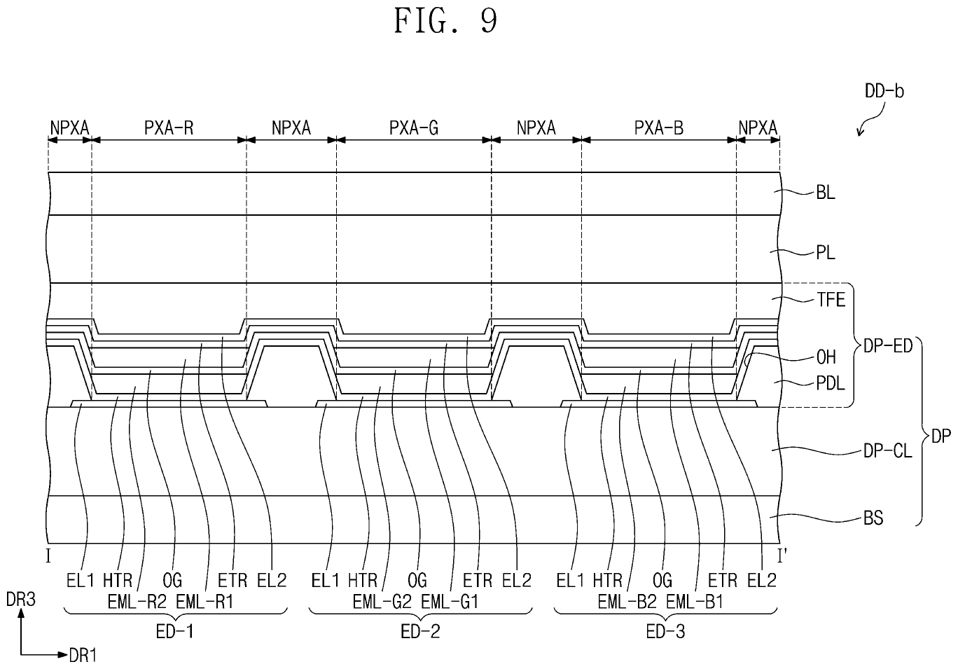

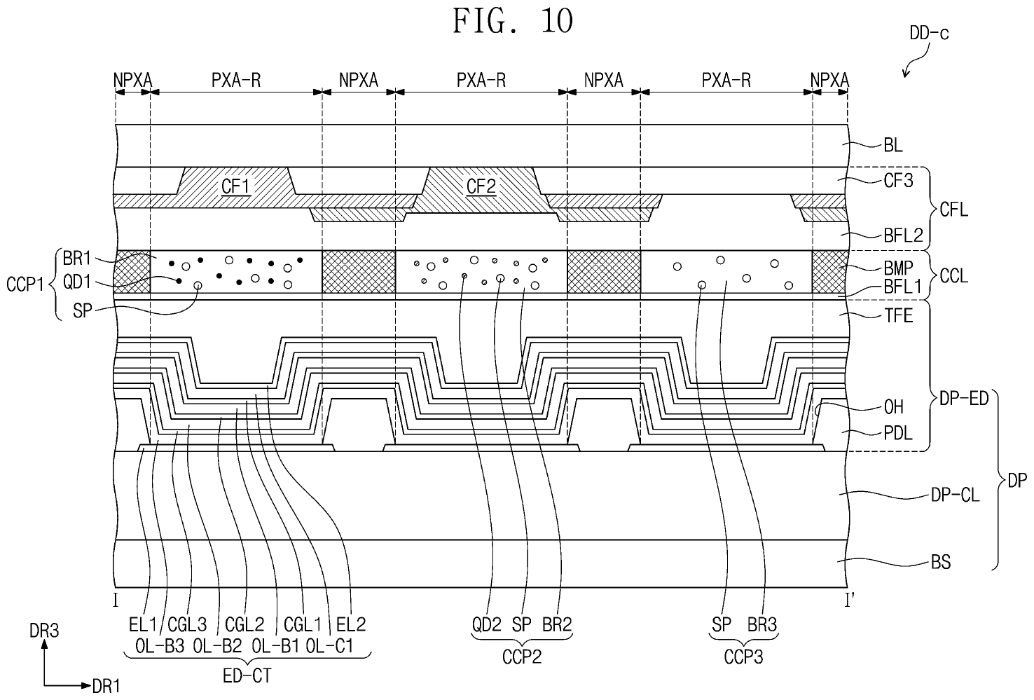

Referring to FIG. 9, the display apparatus DD-b according to an embodiment may include light emitting devices ED-1, ED-2, and ED-3 in which two emission layers are stacked. Compared with the display apparatus DD of an embodiment illustrated in FIG. 2, an embodiment illustrated in FIG. 9 has a difference in that the first to third light emitting devices ED-1, ED-2, and ED-3 each include two emission layers stacked in the thickness direction. In each of the first to third light emitting devices ED-1, ED-2, and ED-3, the two emission layers may emit light in substantially the same wavelength region.

The first light emitting device ED-1 may include a first red emission layer EML-R1 and a second red emission layer EML-R2. The second light emitting device ED-2 may include a first green emission layer EML-G1 and a second green emission layer EML-G2. In some embodiments, the third light emitting device ED-3 may include a first blue emission layer EML-B1 and a second blue emission layer EML-B2. An emission auxiliary part OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting devices ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be between the electron transport region ETR and the emission auxiliary part OG. The second red emission layer EML-R2, the second green emission layer EML-G2, and the second blue emission layer EML-B2 may be between the emission auxiliary part OG and the hole transport region HTR.

For example, the first light emitting device ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked from the first electrode EL1. The second light emitting device ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked from the first electrode EL1. The third light emitting device ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked from the first electrode EL1.

In some embodiments, an optical auxiliary layer PL may be on the display device layer DP-ED. The optical auxiliary layer PL may include a polarizing layer. The optical auxiliary layer PL may be on the display panel DP and control reflected light in the display panel DP due to external light. The optical auxiliary layer PL in the display apparatus according to an embodiment may not be provided.

Unlike FIGS. 8 and 9, FIG. 10 illustrates that a display apparatus DD-c includes four light emitting structures OL-B1, OL-B2, OL-B3, and OL-C1. A light emitting device ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, the embodiment of the present disclosure is not limited thereto, and the first to fourth light emitting structures OL-B1, OL-B2, OL-B3, and OL-C1 may emit light beams in different wavelength regions.

The charge generation layers CGL1, CGL2, and CGL3 disposed between adjacent light emitting structures OL-B1, OL-B2, OL-B3, and OL-C1 may include a p-type (kind) charge generation layer and/or an n-type (kind) charge generation layer.

At least one among the light emitting structures OL-B1, OL-B2, OL-B3, and OL-C1 included in the display apparatus DD-c of an embodiment may include the above-described amine compound of an embodiment.

Hereinafter, with reference to Examples and Comparative Examples, an amine compound according to an embodiment of the present disclosure and a light emitting device of an embodiment of the present disclosure will be described in more detail. In addition, Examples described below are merely illustrations to assist the understanding of the present disclosure, and the scope of the present disclosure is not limited thereto.

### EXAMPLES

### 1. Synthesis of Amine Compound

First, a synthetic method of an amine compound according to the current embodiment will be described in more detail by illustrating synthetic methods of Compounds A10, A127, A179, A199, B32, B135, B151, B182, C35, C52, C98, C341, C361, D9, D53, D68, E14, E164, E193, and E249. Also, in the following descriptions, the synthetic method of the amine compound is provided as an example, but the synthetic method according to an embodiment of the present disclosure is not limited to the examples below. In some embodiments, for the molecular weight of the compound, FAB-MS was measured by using JMS-700V manufactured by JEOL, Ltd. In some embodiments, for the NMR of the compound, 1H-NMR was measured by using AVAVCE300M manufactured by Bruker Biospin K.K.

### (1) Synthesis of Compound A10

Amine Compound A10 may be synthesized by, for example, the reaction below.

### Synthesis of Intermediate IM-1

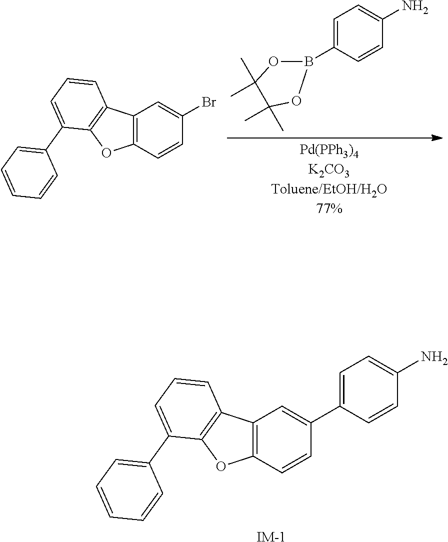

In an Ar atmosphere, in a 2000 mL three-neck flask, 2-bromo-6-phenyldibenzofuran (50.00 g, 154.7 mmol), 4-(4,4,5,5-tetramethyl-1,3,2-dioxaborolan-2-yl)aniline (37.28 g, 1.1 equiv, 170.2 mmol), K₂CO₃ (64.15 g, 3.0 equiv, 464.1 mmol), Pd(PPh₃)₄ (8.94 g, 0.05 equiv, 7.7 mmol), and a mixed solution of toluene/EtOH/H₂O (4/2/1) (1083 mL) were sequentially added, and heated and stirred at about 80° C. After air-cooled to room temperature, the reaction solution was extracted with toluene. A water layer was removed, organic layers were washed with saturated saline, and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-1 (39.96 g, yield 77%).

By measuring FAB-MS, a mass number of m/z=335 was observed by molecular ion peak, thereby identifying Intermediate IM-1.

### Synthesis of Intermediate IM-2

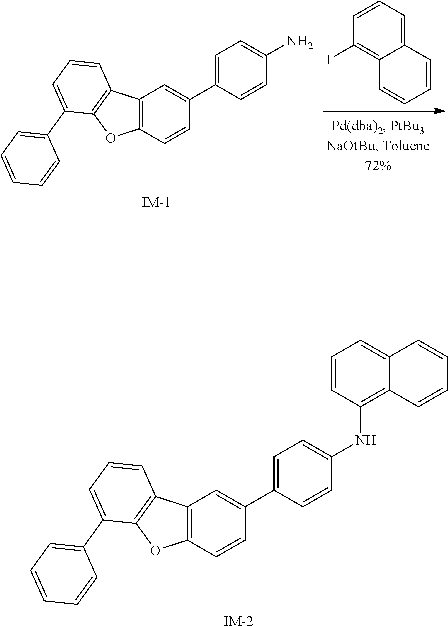

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 1-iodonaphthalene (12.50 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-2 (14.86 g, yield 72%).

By measuring FAB-MS, a mass number of m/z=461 was observed by molecular ion peak, thereby identifying Intermediate IM-2.

### Synthesis of Compound A10

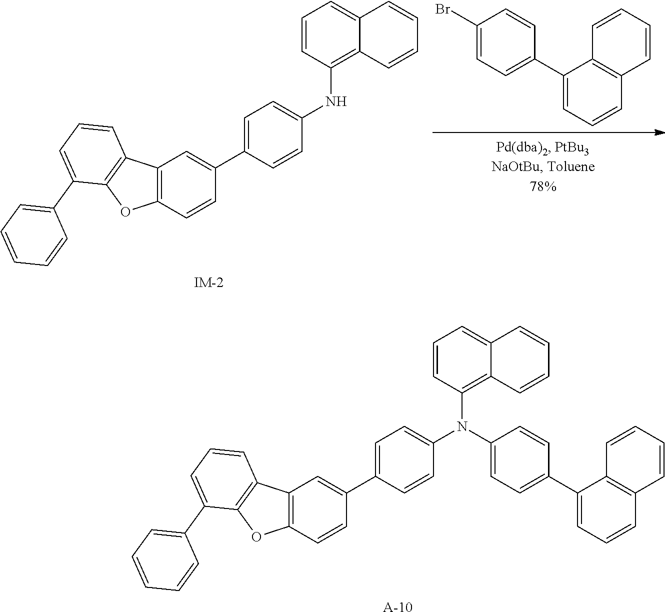

In an Ar atmosphere, in a 300 mL three-neck flask, Intermediate IM-2 (10.00 g, 21.7 mmol), Pd(dba)₂ (0.37 g, 0.03 equiv, 0.6 mmol), NaOtBu (4.16 g, 2.0 equiv, 43.3 mmol), toluene (108 mL), 1-(4-bromophenyl)naphthalene (6.75 g, 1.1 equiv, 23.8 mmol), and PtBu₃ (0.44 g, 0.1 equiv, 2.2 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A10 (11.22 g, yield 78%).

By measuring FAB-MS, a mass number of m/z=663 was observed by molecular ion peak, thereby identifying Compound A10.

### (2) Synthesis of Compound A127

Amine Compound A127 may be synthesized by, for example, the reaction below.

### Synthesis of Intermediate IM-3

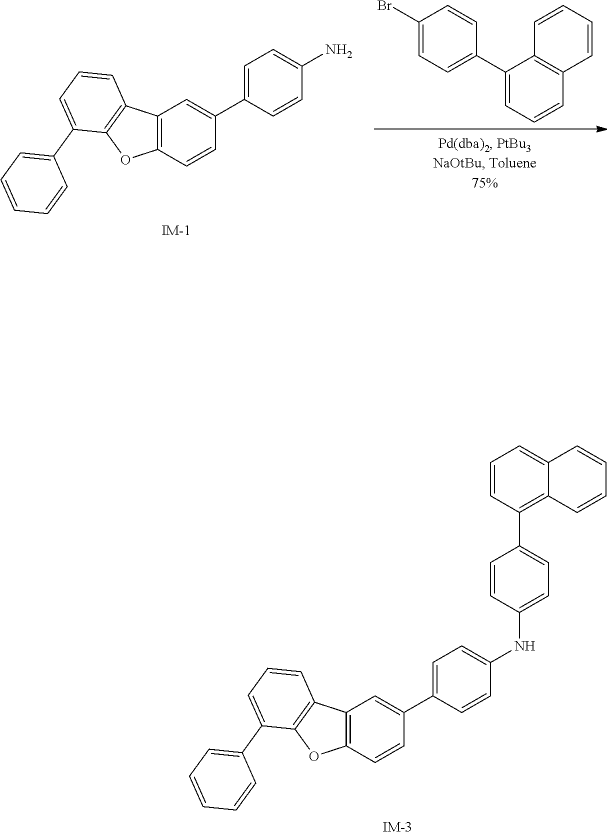

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 1-(4-bromophenyl)naphthalene (13.93 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-3 (18.03 g, yield 75%).

By measuring FAB-MS, a mass number of m/z=537 was observed by molecular ion peak, thereby identifying Intermediate IM-3.

### Synthesis of Compound A127

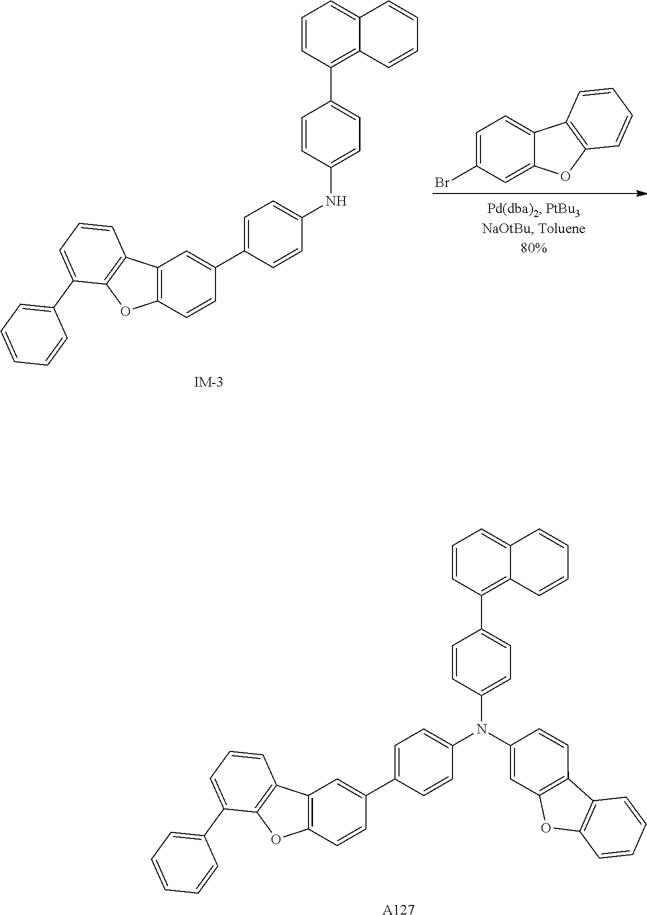

In an Ar atmosphere, in a 300 mL three-neck flask, Intermediate IM-3 (10.00 g, 18.6 mmol), Pd(dba)₂ (0.32 g, 0.03 equiv, 0.6 mmol), NaOtBu (3.57 g, 2.0 equiv, 37.2 mmol), toluene (93 mL), 3-bromodibenzofuran (5.06 g, 1.1 equiv, 20.5 mmol), and PtBu₃ (0.38 g, 0.1 equiv, 1.9 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A127 (10.47 g, yield 80%).

By measuring FAB-MS, a mass number of m/z=703 was observed by molecular ion peak, thereby identifying Compound A127.

### (3) Synthesis of Compound A179

Amine Compound A179 may be synthesized by, for example, the reaction below.

### Synthesis of Intermediate IM-4

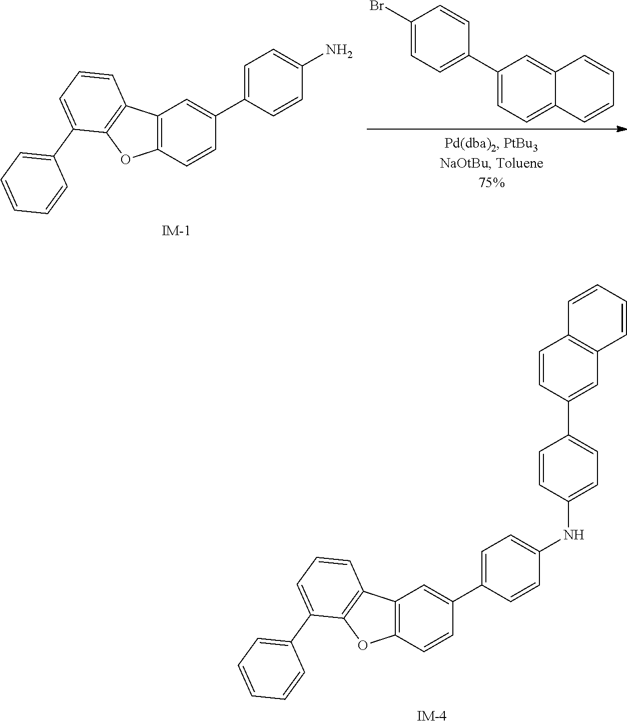

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 2-(4-bromophenyl)naphthalene (13.93 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-4 (18.03 g, yield 75%).

By measuring FAB-MS, a mass number of m/z=537 was observed by molecular ion peak, thereby identifying Intermediate IM-4.

### Synthesis of Compound A179

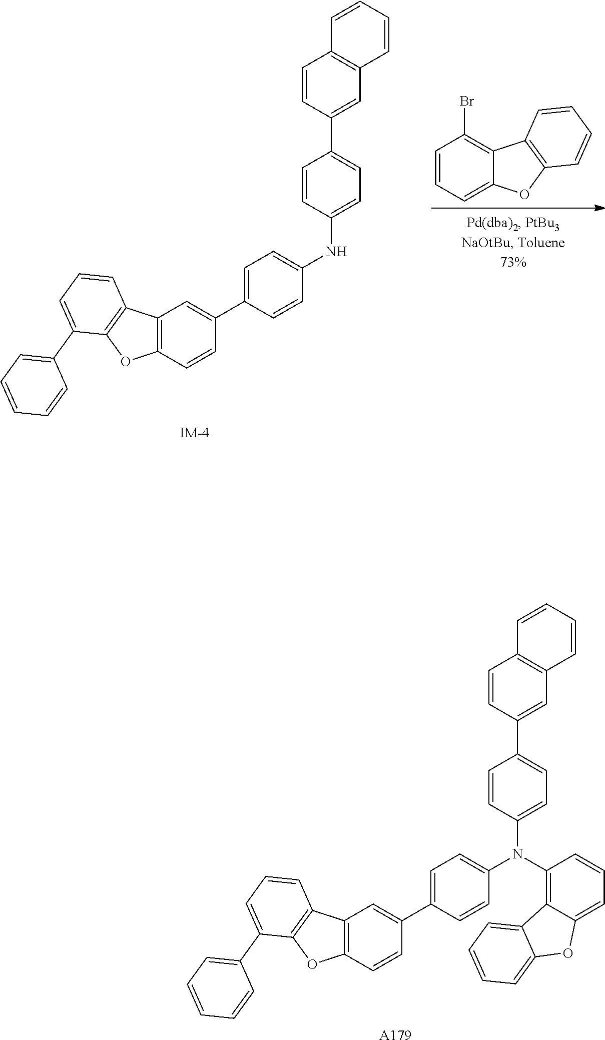

In an Ar atmosphere, in a 300 mL three-neck flask, Intermediate IM-4 (10.00 g, 18.6 mmol), Pd(dba)₂ (0.32 g, 0.03 equiv, 0.6 mmol), NaOtBu (3.57 g, 2.0 equiv, 37.2 mmol), toluene (93 mL), 1-bromodibenzofuran (5.06 g, 1.1 equiv, 20.5 mmol), and PtBu₃ (0.38 g, 0.1 equiv, 1.9 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A179 (9.56 g, yield 73%).

By measuring FAB-MS, a mass number of m/z=703 was observed by molecular ion peak, thereby identifying Compound A179.

### (4) Synthesis of Compound A199

Amine Compound A199 may be synthesized by, for example, the reaction below.

### Synthesis of Compound A199

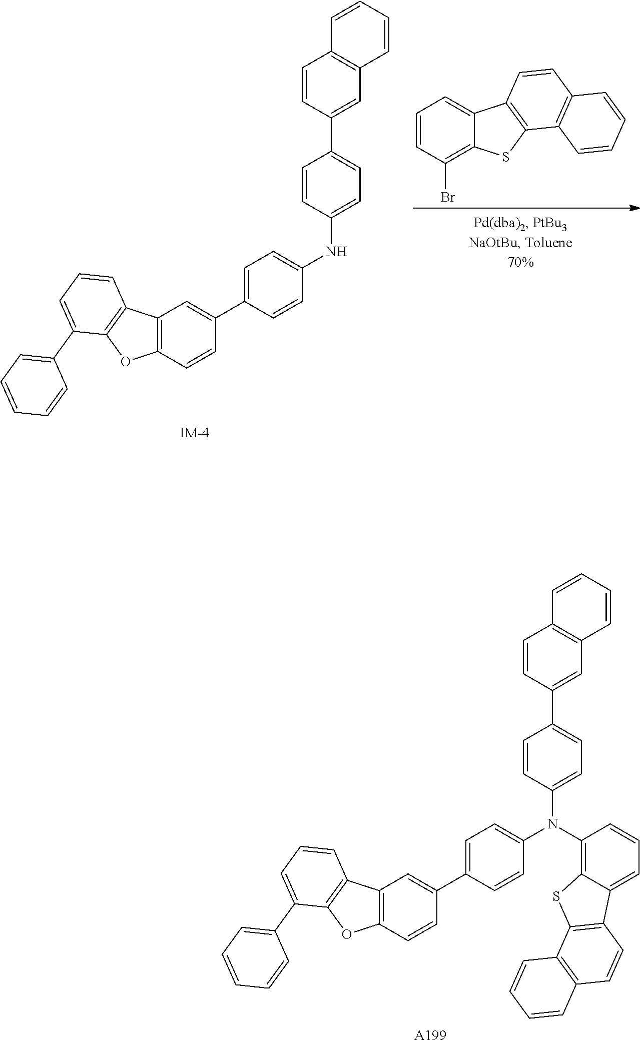

In an Ar atmosphere, in a 300 mL three-neck flask, Intermediate IM-4 (10.00 g, 18.6 mmol), Pd(dba)₂ (0.32 g, 0.03 equiv, 0.6 mmol), NaOtBu (3.57 g, 2.0 equiv, 37.2 mmol), toluene (93 mL), 10-bromobenzonaphtho[2,1-d]thiophene (6.47 g, 1.1 equiv, 20.5 mmol), and PtBu₃ (0.38 g, 0.1 equiv, 1.9 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound A199 (10.02 g, yield 70%).

By measuring FAB-MS, a mass number of m/z=769 was observed by molecular ion peak, thereby identifying Compound A199.

### (5) Synthesis of Compound B32

Amine Compound B32 may be synthesized by, for example, the reaction below.

### Synthesis of Intermediate IM-5

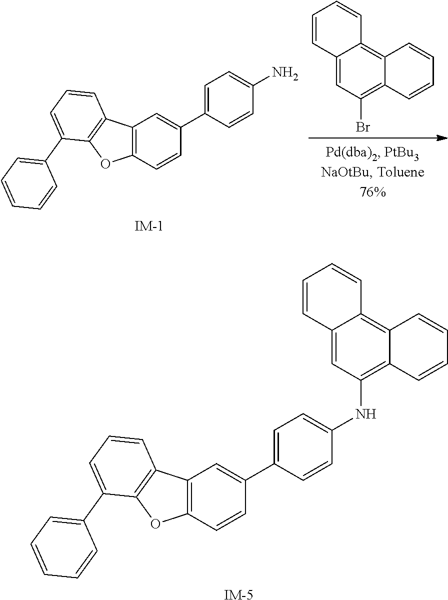

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 9-bromophenanthrene (12.65 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-5 (17.39 g, yield 76%).

By measuring FAB-MS, a mass number of m/z=511 was observed by molecular ion peak, thereby identifying Intermediate IM-5.

### Synthesis of Compound B32

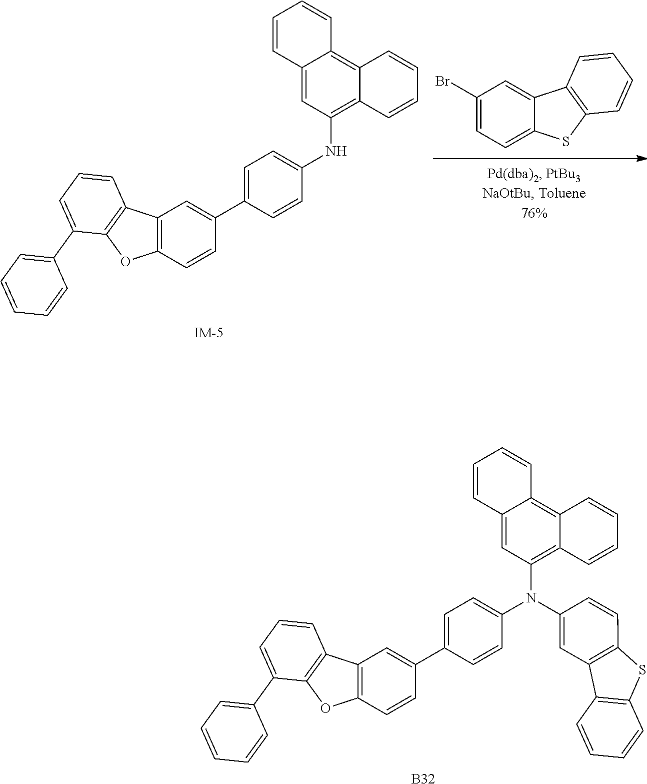

In an Ar atmosphere, in a 300 mL three-neck flask, Intermediate IM-5 (10.00 g, 19.5 mmol), Pd(dba)₂ (0.34 g, 0.03 equiv, 0.6 mmol), NaOtBu (3.76 g, 2.0 equiv, 39.1 mmol), toluene (98 mL), 2-bromodibenzothiophene (5.68 g, 1.1 equiv, 21.5 mmol), and PtBu₃ (0.40 g, 0.1 equiv, 2.0 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B32 (10.31 g, yield 76%).

By measuring FAB-MS, a mass number of m/z=693 was observed by molecular ion peak, thereby identifying Compound B32.

### (6) Synthesis of Compound B135

Amine Compound B135 may be synthesized by, for example, the reaction below.

### Synthesis of Intermediate IM-6

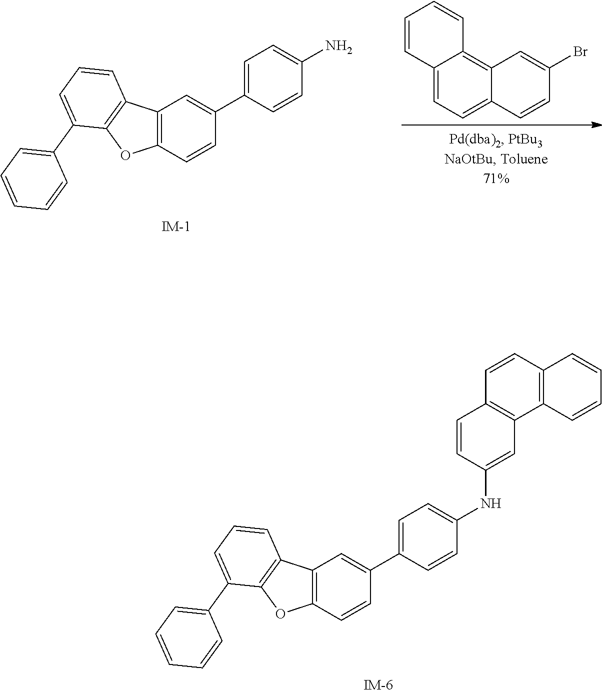

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 3-bromophenanthrene (12.65 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-6 (16.25 g, yield 71%).

By measuring FAB-MS, a mass number of m/z=511 was observed by molecular ion peak, thereby identifying Intermediate IM-6.

### Synthesis of Compound B135

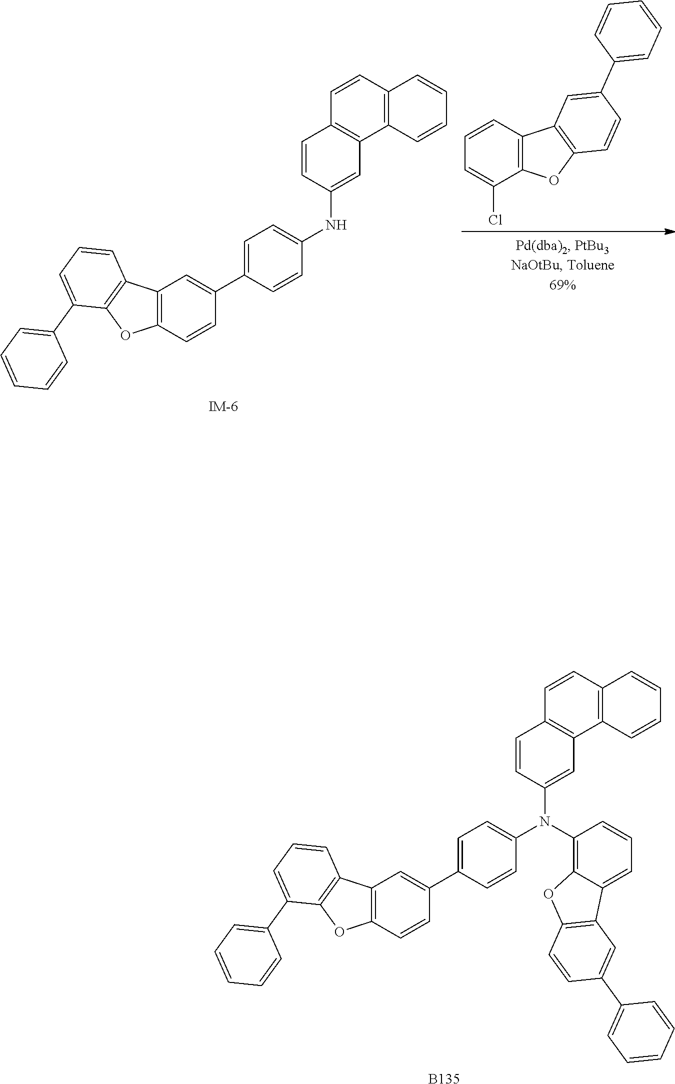

In an Ar atmosphere, in a 300 mL three-neck flask, Intermediate IM-6 (10.00 g, 19.5 mmol), Pd(dba)₂ (0.34 g, 0.03 equiv, 0.6 mmol), NaOtBu (3.76 g, 2.0 equiv, 39.1 mmol), toluene (98 mL), 6-chloro-2-phenyldibenzofuran (5.99 g, 1.1 equiv, 21.5 mmol), and PtBu₃ (0.40 g, 0.1 equiv, 2.0 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B135 (10.17 g, yield 69%).

By measuring FAB-MS, a mass number of m/z=753 was observed by molecular ion peak, thereby identifying Compound B135.

### (7) Synthesis of Compound B151

Amine Compound B151 may be synthesized by, for example, the reaction below.

### Synthesis of Intermediate IM-7

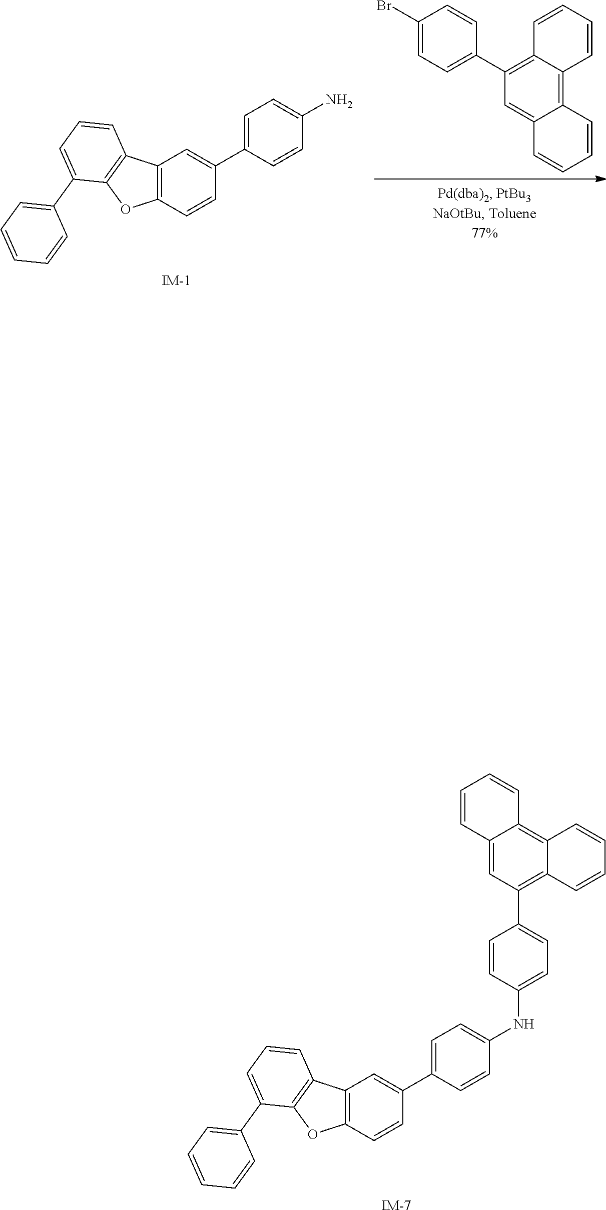

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 9-(4-bromophenyl)phenanthrene (16.39 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-7 (20.24 g, yield 77%).

By measuring FAB-MS, a mass number of m/z=587 was observed by molecular ion peak, thereby identifying Intermediate IM-7.

### Synthesis of Compound B151

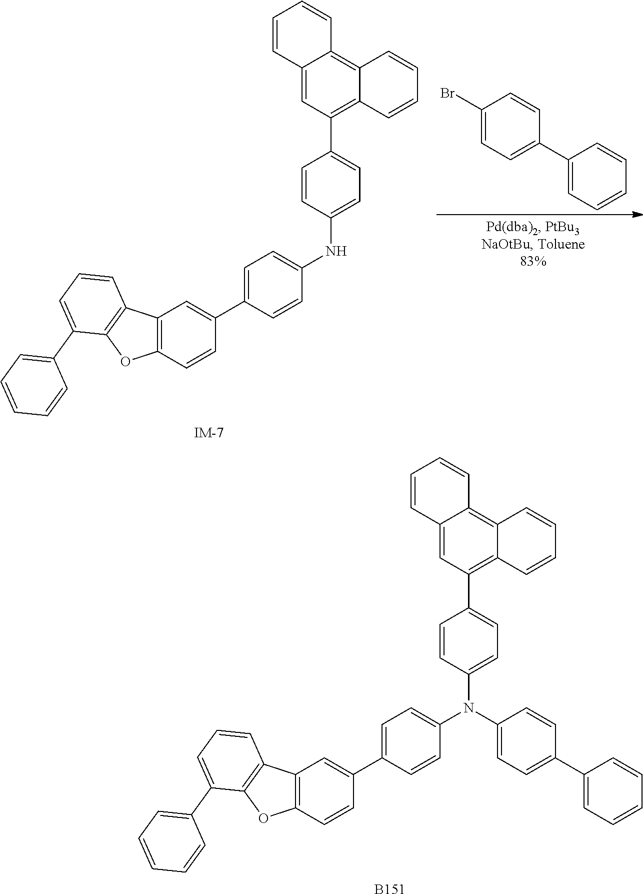

In an Ar atmosphere, in a 300 mL three-neck flask, Intermediate IM-7 (10.00 g, 17.0 mmol), Pd(dba)₂ (0.29 g, 0.03 equiv, 0.5 mmol), NaOtBu (3.27 g, 2.0 equiv, 34.0 mmol), toluene (85 mL), 4-bromobiphenyl (4.37 g, 1.1 equiv, 18.7 mmol), and PtBu₃ (0.34 g, 0.1 equiv, 1.7 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound B151 (10.45 g, yield 83%).

By measuring FAB-MS, a mass number of m/z=739 was observed by molecular ion peak, thereby identifying Compound B151.

### (8) Synthesis of Compound B182

Amine Compound B182 may be synthesized by, for example, the reaction below.

### Synthesis of Intermediate IM-8

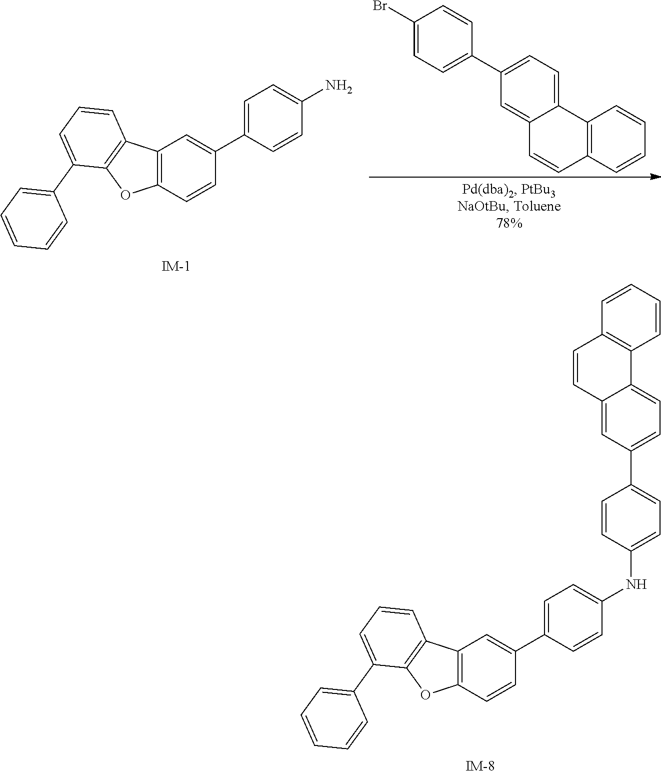

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 2-(4-bromophenyl)phenanthrene (16.39 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, the organic layer was fractionated by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-8 (20.50 g, yield 78%).

By measuring FAB-MS, a mass number of m/z=587 was observed by molecular ion peak, thereby identifying Intermediate IM-8.

### Synthesis of Compound B182

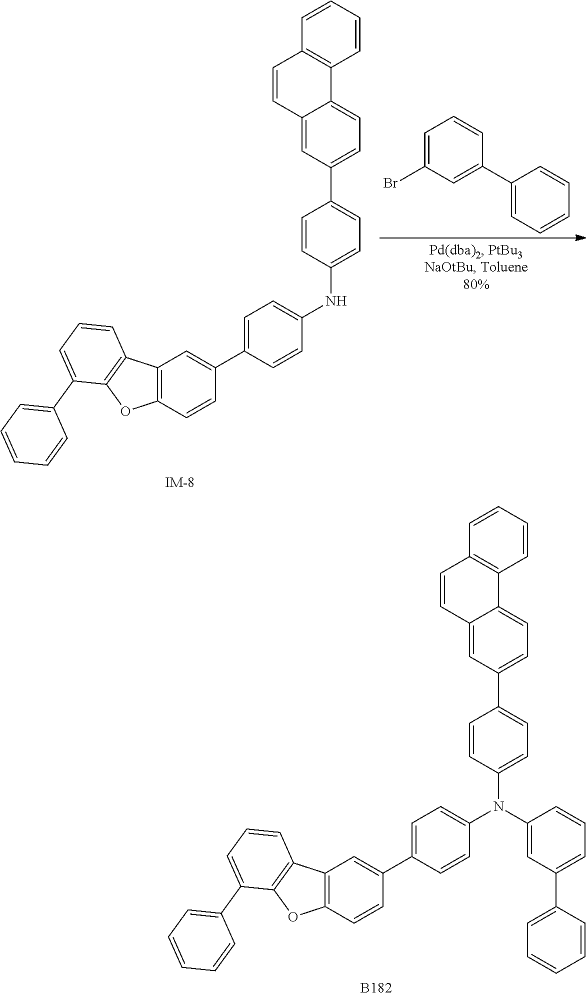

In an Ar atmosphere, in a 300 mL three-neck flask, Intermediate IM-8 (10.00 g, 17.0 mmol), Pd(dba)₂ (0.29 g, 0.03 equiv, 0.5 mmol), NaOtBu (3.27 g, 2.0 equiv, 34.0 mmol), toluene (85 mL), 3-bromobiphenyl (4.37 g, 1.1 equiv, 18.7 mmol), and PtBu₃ (0.34 g, 0.1 equiv, 1.7 mmol) were sequentially added, and then heated and stirred under reflux. After the reaction solution was air-cooled to room temperature, the organic layer was fractionated by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound B182 (10.07 g, yield 80%).

By measuring FAB-MS, a mass number of m/z=739 was observed by molecular ion peak, thereby identifying Compound B182.

### (9) Synthesis of Compound C35

Amine Compound C35 may be synthesized by, for example, the reaction below.

### Synthesis of Intermediate IM-9

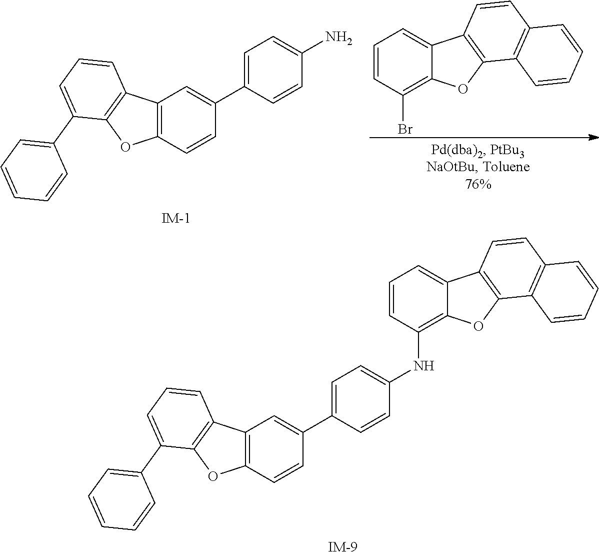

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 10-bromonaphtho[1,2-b]benzofuran (14.62 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-9 (18.75 g, yield 76%).

By measuring FAB-MS, a mass number of m/z=551 was observed by molecular ion peak, thereby identifying Intermediate IM-9.

### Synthesis of Compound C35

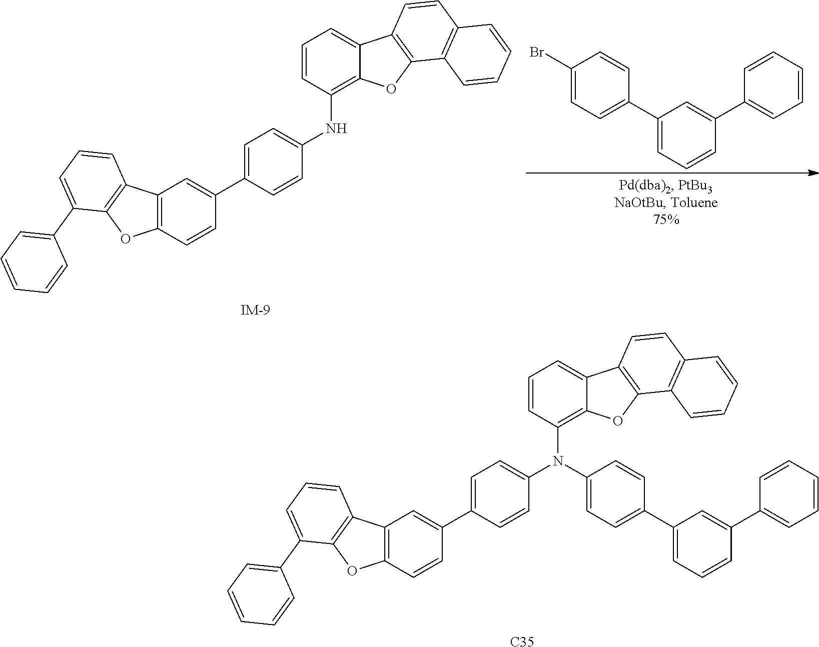

In an Ar atmosphere, in a 300 mL three-neck flask, Intermediate IM-9 (10.00 g, 18.1 mmol), Pd(dba)₂ (0.31 g, 0.03 equiv, 0.5 mmol), NaOtBu (3.48 g, 2.0 equiv, 36.3 mmol), toluene (90 mL), 4-bromo-1,1′:3′,1″-terphenyl (6.17 g, 1.1 equiv, 19.9 mmol), and PtBu₃ (0.37 g, 0.1 equiv, 1.8 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C35 (10.60 g, yield 75%).

By measuring FAB-MS, a mass number of m/z=779 was observed by molecular ion peak, thereby identifying Compound C35.

### (10) Synthesis of Compound C52

Amine Compound C52 may be synthesized by, for example, the reaction below.

### Synthesis of Intermediate IM-10

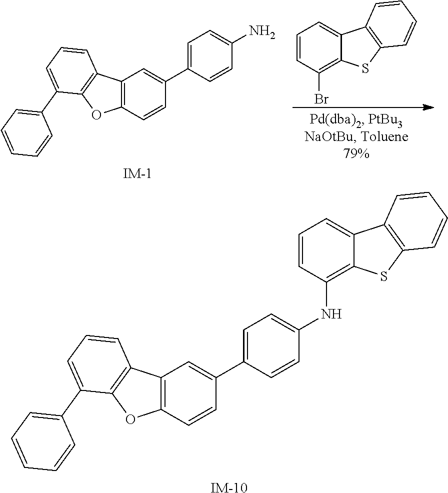

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 4-bromodibenzothiophene (12.95 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-10 (18.29 g, yield 79%).

By measuring FAB-MS, a mass number of m/z=517 was observed by molecular ion peak, thereby identifying Intermediate IM-10.

### Synthesis of Compound C52

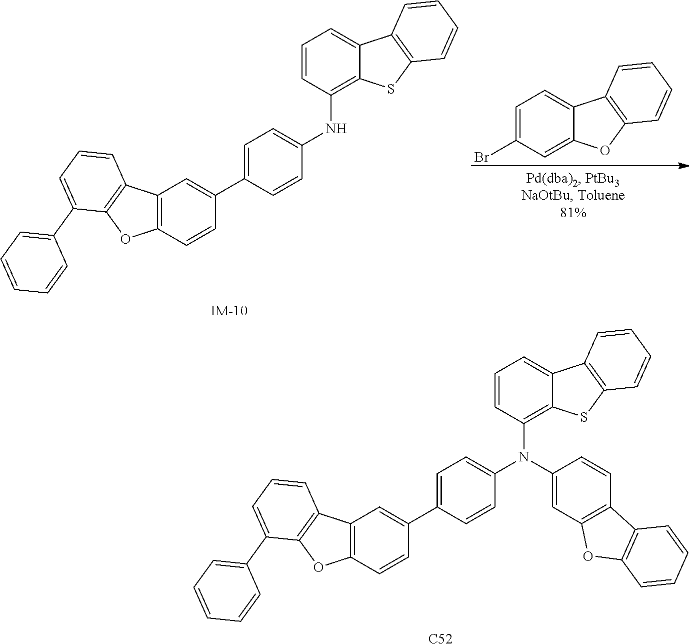

In an Ar atmosphere, in a 300 mL three-neck flask, Intermediate IM-10 (10.00 g, 19.3 mmol), Pd(dba)₂ (0.33 g, 0.03 equiv, 0.6 mmol), NaOtBu (3.71 g, 2.0 equiv, 38.6 mmol), toluene (97 mL), 3-bromodibenzofuran (5.25 g, 1.1 equiv, 21.2 mmol), and PtBu₃ (0.39 g, 0.1 equiv, 1.9 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C52 (10.70 g, yield 81%).

By measuring FAB-MS, a mass number of m/z=683 was observed by molecular ion peak, thereby identifying Compound C52.

### (11) Synthesis of Compound C98

Amine Compound C98 may be synthesized by, for example, the reaction below.

### Synthesis of Intermediate IM-11

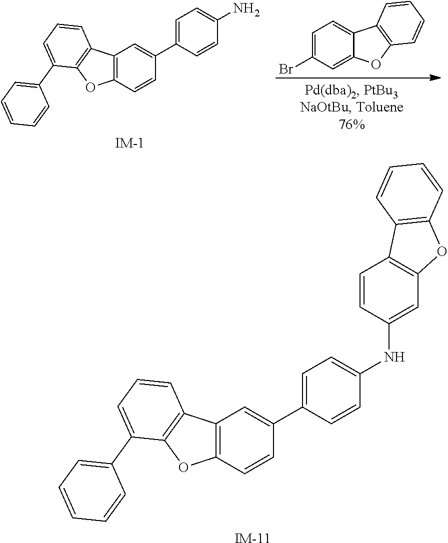

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 3-bromodibenzofuran (12.16 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-11 (17.05 g, yield 76%).

By measuring FAB-MS, a mass number of m/z=501 was observed by molecular ion peak, thereby identifying Intermediate IM-11.

### Synthesis of Compound C98

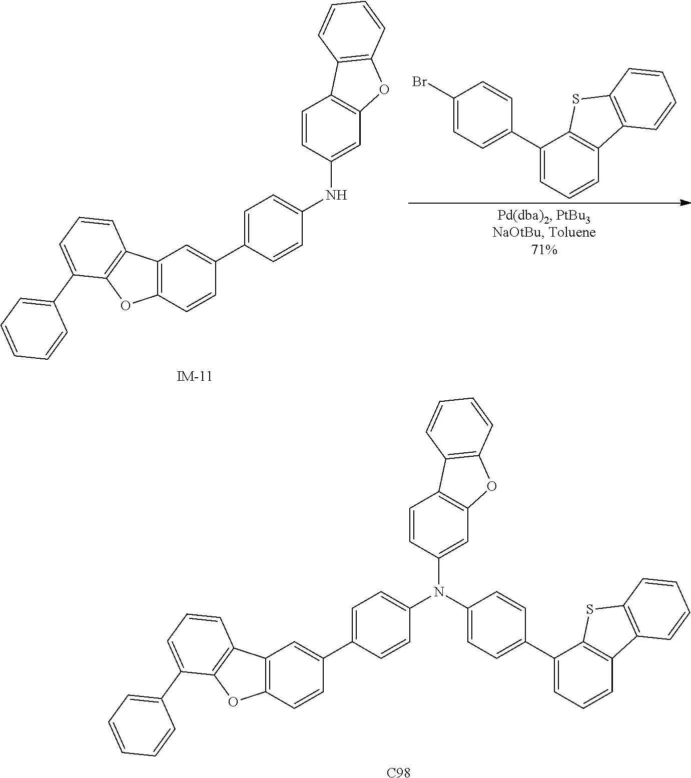

In an Ar atmosphere, in a 300 mL three-neck flask, Intermediate IM-11 (10.00 g, 19.9 mmol), Pd(dba)₂ (0.34 g, 0.03 equiv, 0.6 mmol), NaOtBu (3.83 g, 2.0 equiv, 39.9 mmol), toluene (100 mL), 4-(4-bromophenyl)dibenzothiophene (7.44 g, 1.1 equiv, 21.9 mmol), and PtBu₃ (0.40 g, 0.1 equiv, 2.0 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C98 (10.76 g, yield 71%).

By measuring FAB-MS, a mass number of m/z=759 was observed by molecular ion peak, thereby identifying Compound C98.

### (12) Synthesis of Compound C341

Amine Compound C341 may be synthesized by, for example, the reaction below.

### Synthesis of Intermediate IM-12

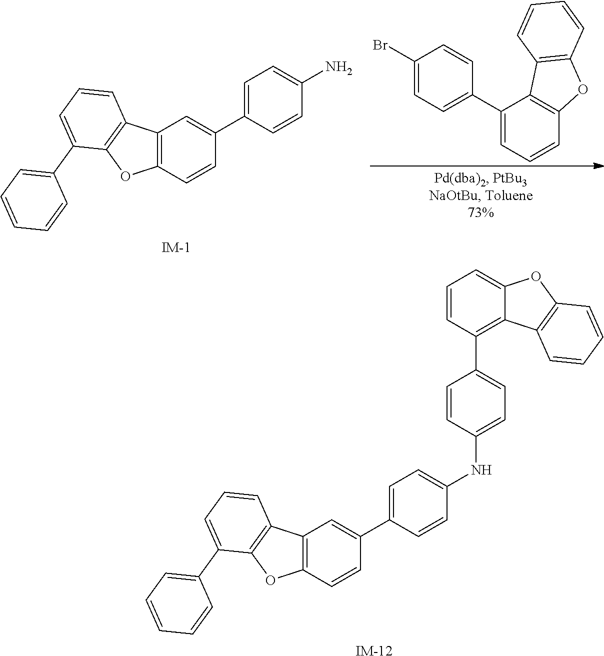

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 1-(4-bromophenyl)dibenzofuran (15.90 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-12 (18.86 g, yield 73%).

By measuring FAB-MS, a mass number of m/z=577 was observed by molecular ion peak, thereby identifying Intermediate IM-12.

### Synthesis of Compound C341

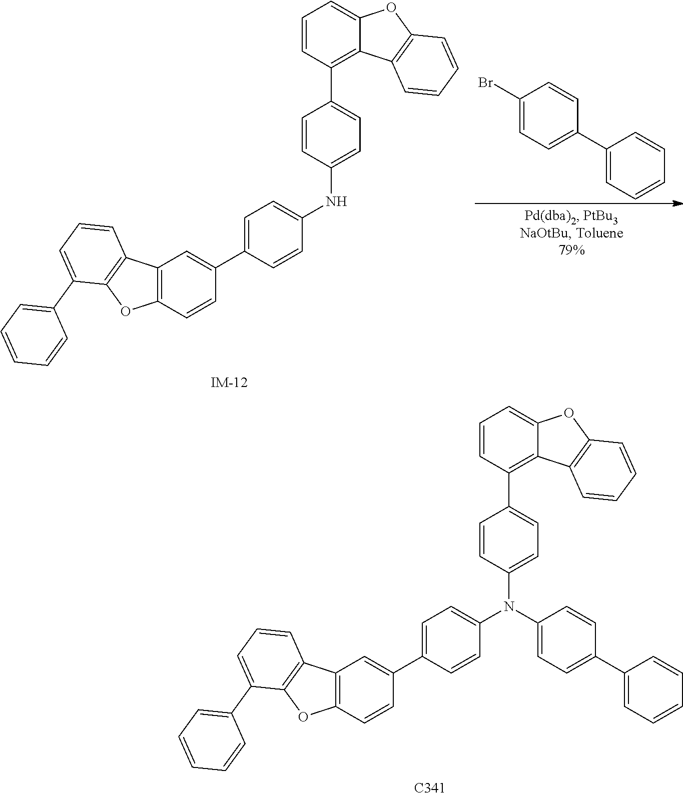

In an Ar atmosphere, in a 300 mL three-neck flask, Intermediate IM-12 (10.00 g, 17.3 mmol), Pd(dba)₂ (0.30 g, 0.03 equiv, 0.5 mmol), NaOtBu (3.33 g, 2.0 equiv, 34.6 mmol), toluene (87 mL), 4-bromobiphenyl (4.44 g, 1.1 equiv, 19.0 mmol), and PtBu₃ (0.35 g, 0.1 equiv, 1.7 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C341 (9.98 g, yield 79%).

By measuring FAB-MS, a mass number of m/z=729 was observed by molecular ion peak, thereby identifying Compound C341.

### (13) Synthesis of Compound C361

Amine Compound C361 may be synthesized by, for example, the reaction below.

### Synthesis of Intermediate IM-13

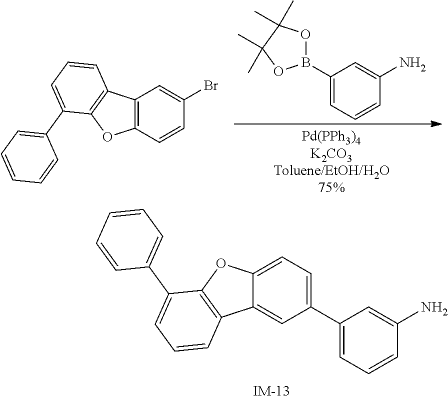

In an Ar atmosphere, in a 2000 mL three-neck flask, 2-bromo-6-phenyldibenzofuran (50.00 g, 154.7 mmol), 3-(4,4,5,5-tetramethyl-1,3,2-dioxaborolan-2-yl)aniline (37.28 g, 1.1 equiv, 170.2 mmol), K₂CO₃ (64.15 g, 3.0 equiv, 464.1 mmol), Pd(PPh₃)₄ (8.94 g, 0.05 equiv, 7.7 mmol), and a mixed solution of toluene/EtOH/H₂O (4/2/1) (1083 mL) were sequentially added, and heated and stirred at about 80° C. After air-cooled to room temperature, the reaction solution was extracted with toluene. A water layer was removed, organic layers were washed with saturated saline, and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-13 (38.92 g, yield 75%).

By measuring FAB-MS, a mass number of m/z=335 was observed by molecular ion peak, thereby identifying Intermediate IM-13.

### Synthesis of Intermediate IM-14

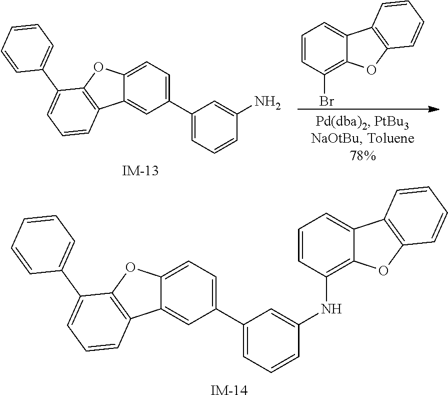

In an Ar atmosphere, in a 500 mL three-neck flask, Intermediate IM-13 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 4-bromodibenzofuran (12.16 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-14 (16.82 g, yield 75%).

By measuring FAB-MS, a mass number of m/z=501 was observed by molecular ion peak, thereby identifying Intermediate IM-14.

### Synthesis of Compound C361

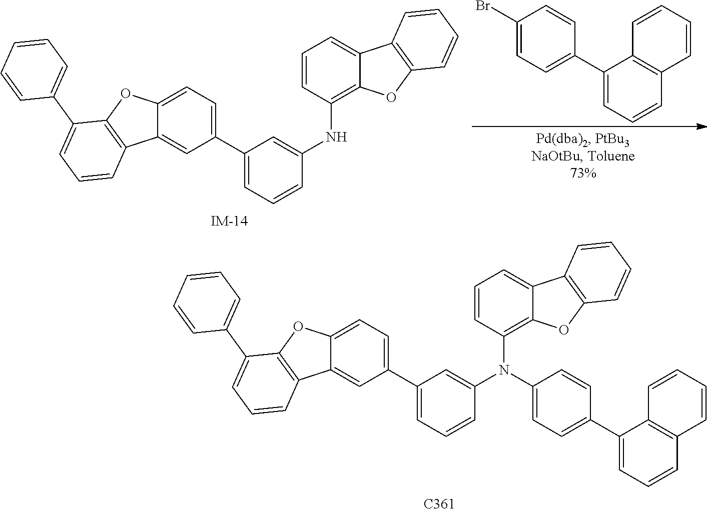

In an Ar atmosphere, in a 300 mL three-neck flask, Intermediate IM-14 (10.00 g, 19.9 mmol), Pd(dba)₂ (0.34 g, 0.03 equiv, 0.6 mmol), NaOtBu (3.83 g, 2.0 equiv, 39.9 mmol), toluene (100 mL), 1-(4-bromophenyl)naphthalene (6.21 g, 1.1 equiv, 21.9 mmol), and PtBu₃ (0.40 g, 0.1 equiv, 2.0 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound C361 (10.24 g, yield 73%).

By measuring FAB-MS, a mass number of m/z=703 was observed by molecular ion peak, thereby identifying Compound C361.

### (14) Synthesis of Compound D9

Amine Compound D9 may be synthesized by, for example, the reaction below.

### Synthesis of Intermediate IM-15

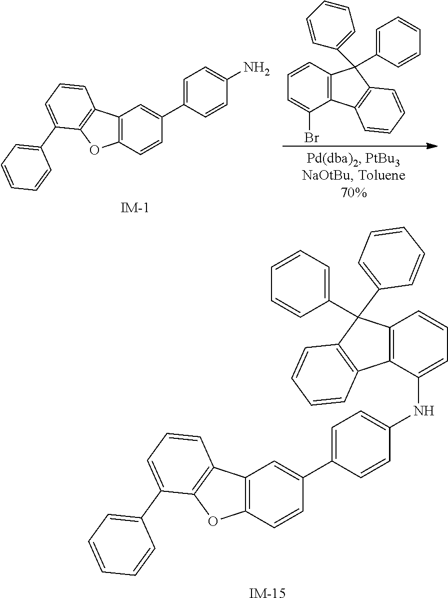

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 4-bromo-9,9-diphenyl-9H-fluorene (19.55 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-15 (20.40 g, yield 70%).

By measuring FAB-MS, a mass number of m/z=651 was observed by molecular ion peak, thereby identifying Intermediate IM-15.

### Synthesis of Compound D9

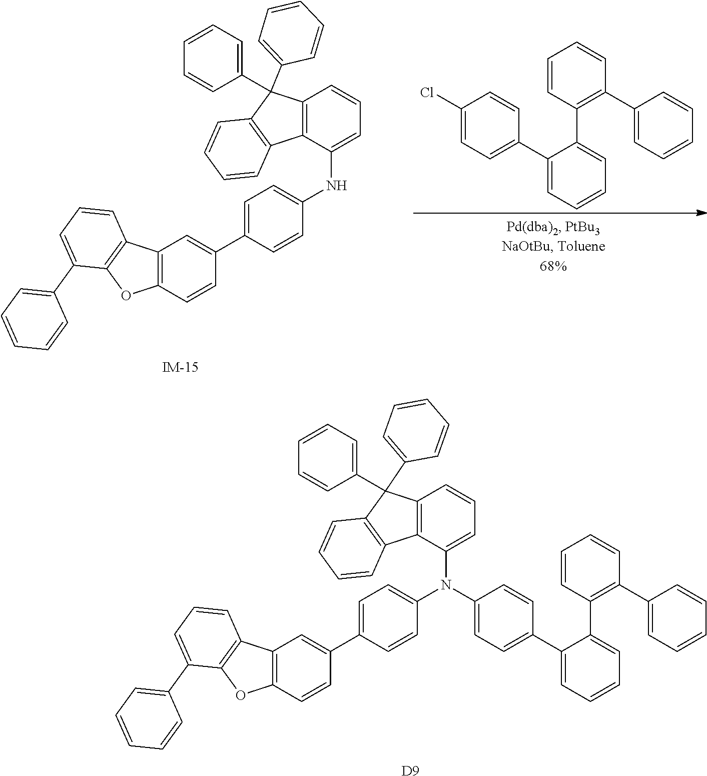

In an Ar atmosphere, in a 300 mL three-neck flask, Intermediate IM-15 (10.00 g, 15.3 mmol), Pd(dba)₂ (0.26 g, 0.03 equiv, 0.5 mmol), NaOtBu (2.95 g, 2.0 equiv, 30.7 mmol), toluene (77 mL), 4-chloro-1,1′:2′,1″:2″,1′″-quaterphenyl (5.75 g, 1.1 equiv, 16.9 mmol), and PtBu₃ (0.31 g, 0.1 equiv, 1.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound D9 (9.96 g, yield 68%).

By measuring FAB-MS, a mass number of m/z=956 was observed by molecular ion peak, thereby identifying Compound D9.

### (15) Synthesis of Compound D53

Amine Compound D53 may be synthesized by, for example, the reaction below.

### Synthesis of Intermediate IM-16

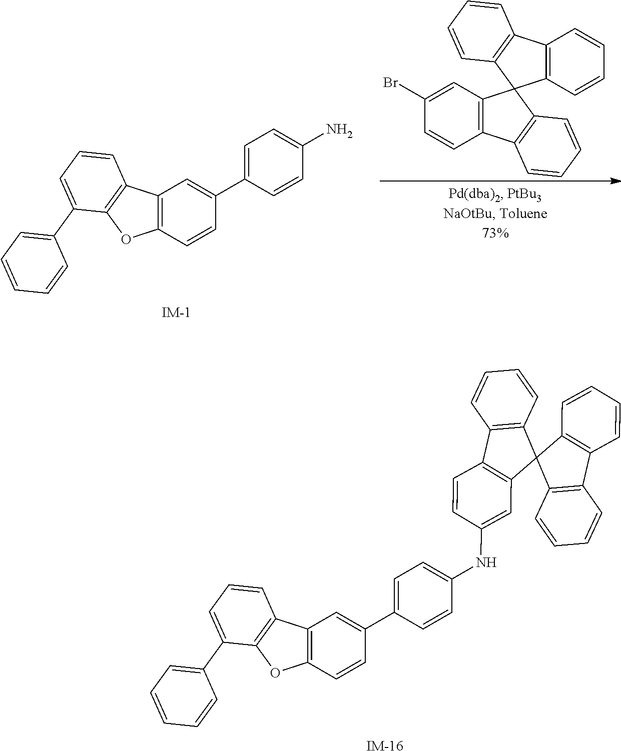

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 2-bromo-9,9′-spirobi[fluorene] (19.45 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-16 (21.21 g, yield 73%).

By measuring FAB-MS, a mass number of m/z=649 was observed by molecular ion peak, thereby identifying Intermediate IM-16.

### Synthesis of Compound D53

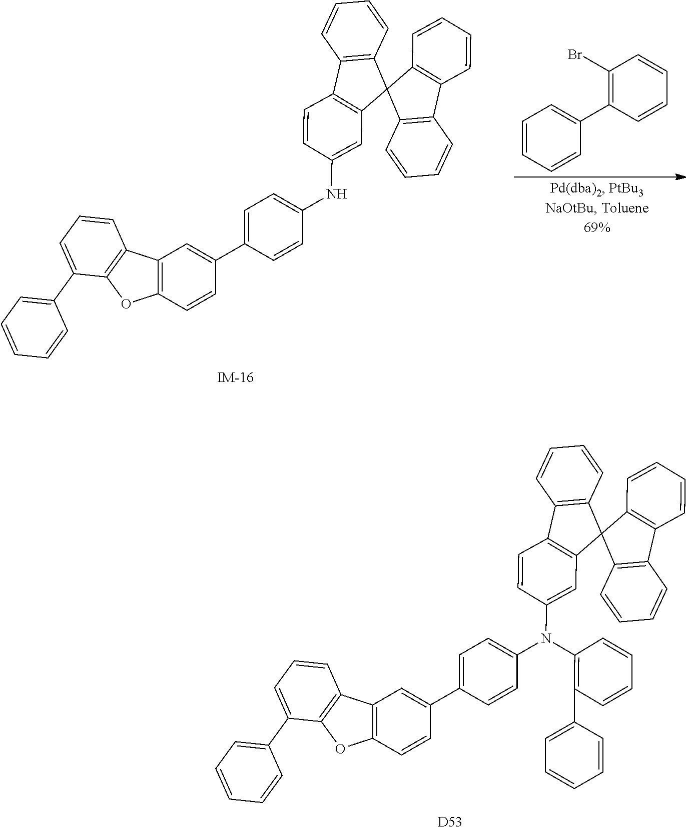

In an Ar atmosphere, in a 300 mL three-neck flask, Intermediate IM-16 (10.00 g, 15.4 mmol), Pd(dba)₂ (0.27 g, 0.03 equiv, 0.5 mmol), NaOtBu (2.96 g, 2.0 equiv, 30.8 mmol), toluene (77 mL), 2-bromobiphenyl (3.95 g, 1.1 equiv, 16.9 mmol), and PtBu₃ (0.31 g, 0.1 equiv, 1.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound D53 (8.62 g, yield 69%).

By measuring FAB-MS, a mass number of m/z=801 was observed by molecular ion peak, thereby identifying Compound D53.

### (16) Synthesis of Compound D68

Amine Compound D68 may be synthesized by, for example, the reaction below.

### Synthesis of Intermediate IM-17

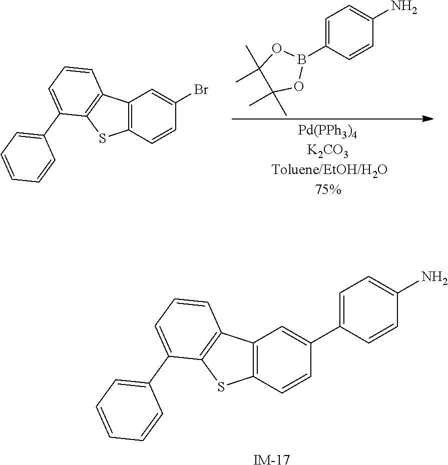

In an Ar atmosphere, in a 2000 mL three-neck flask, 2-bromo-6-phenyldibenzothiophene (50.00 g, 147.4 mmol), 4-(4,4,5,5-tetramethyl-1,3,2-dioxaborolan-2-yl)aniline (35.52 g, 1.1 equiv, 162.1 mmol), K₂CO₃ (61.11 g, 3.0 equiv, 442.2 mmol), Pd(PPh₃)₄ (8.52 g, 0.05 equiv, 7.4 mmol), and a mixed solution of toluene/EtOH/H₂O (4/2/1) (1083 mL) were sequentially added, and heated and stirred at about 80° C. After air-cooled to room temperature, the reaction solution was extracted with toluene. A water layer was removed, organic layers were washed with saturated saline, and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-17 (38.85 g, yield 75%).

By measuring FAB-MS, a mass number of m/z=351 was observed by molecular ion peak, thereby identifying Intermediate IM-17.

### Synthesis of Intermediate IM-18

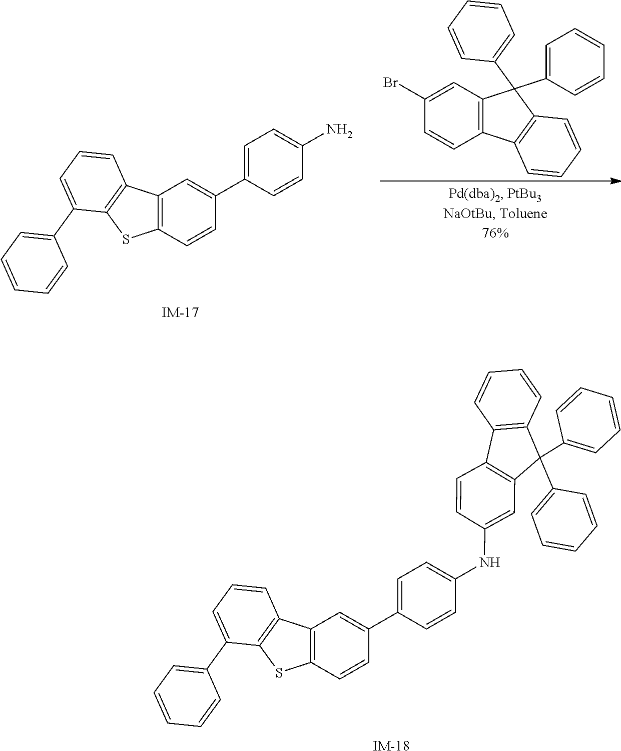

In an Ar atmosphere, in a 500 mL three-neck flask, Intermediate IM-17 (15.00 g, 42.7 mmol), Pd(dba)₂ (0.74 g, 0.03 equiv, 1.3 mmol), NaOtBu (4.10 g, 1.0 equiv, 42.7 mmol), toluene (214 mL), 2-bromo-9,9-diphenyl-9H-fluorene (18.65 g, 1.1 equiv, 46.9 mmol), and PtBu₃ (0.86 g, 0.1 equiv, 4.3 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-18 (21.66 g, yield 76%).

By measuring FAB-MS, a mass number of m/z=667 was observed by molecular ion peak, thereby identifying Intermediate IM-18.

### Synthesis of Compound D68

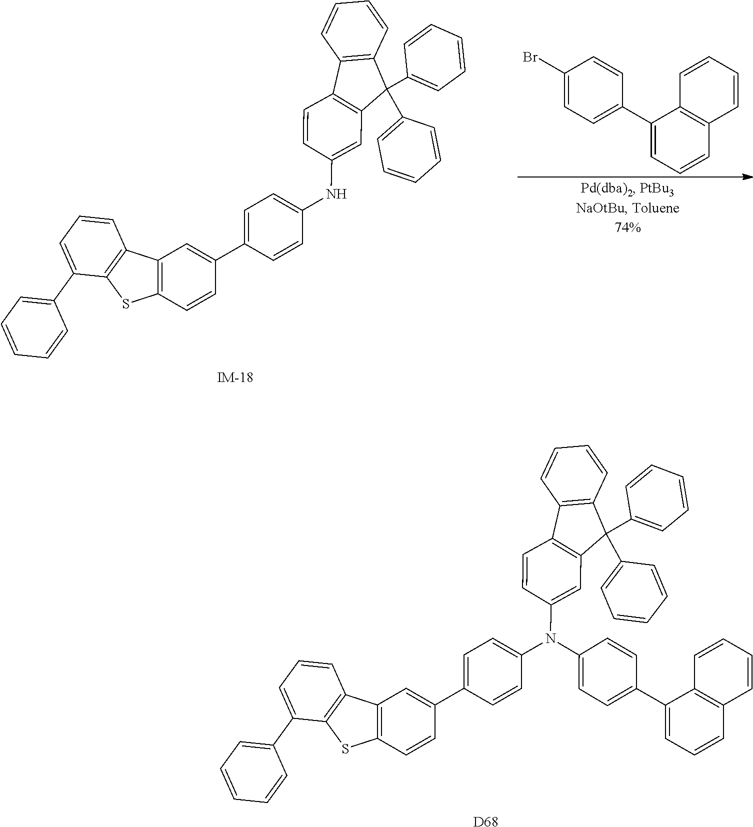

In an Ar atmosphere, in a 300 mL three-neck flask, Intermediate IM-18 (10.00 g, 15.0 mmol), Pd(dba)₂ (0.26 g, 0.03 equiv, 0.5 mmol), NaOtBu (2.88 g, 2.0 equiv, 29.9 mmol), toluene (75 mL), 1-(4-bromophenyl)naphthalene (4.66 g, 1.1 equiv, 16.5 mmol), and PtBu₃ (0.30 g, 0.1 equiv, 1.5 mmol) were sequentially added, and then heated and stirred under reflux. After the reaction solution was air-cooled to room temperature, the organic layer was fractionated by adding water to the reaction solvent. The organic layers were further extracted by adding toluene to a water layer, and then the combined organic layers were washed with saline and dried over MgSO₄. MgSO₄ was filtered off and the organic layers were concentrated, and then the resulting crude product was purified by silica gel column chromatography (using a mixed solvent of hexane and toluene as an eluent) to obtain solid Compound D68 (9.64 g, yield 74%).

By measuring FAB-MS, a mass number of m/z=870 was observed by molecular ion peak, thereby identifying Compound D68.

### (17) Synthesis of Compound E14

Amine Compound E14 may be synthesized by, for example, the reaction below.

### Synthesis of Intermediate IM-19

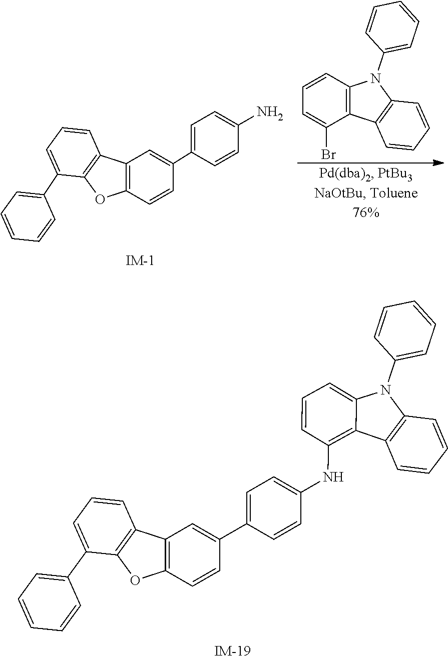

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 4-bromo-9-phenyl-9H-carbazole (15.85 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-19 (19.60 g, yield 76%).

By measuring FAB-MS, a mass number of m/z=576 was observed by molecular ion peak, thereby identifying Intermediate IM-19.

### Synthesis of Compound E14

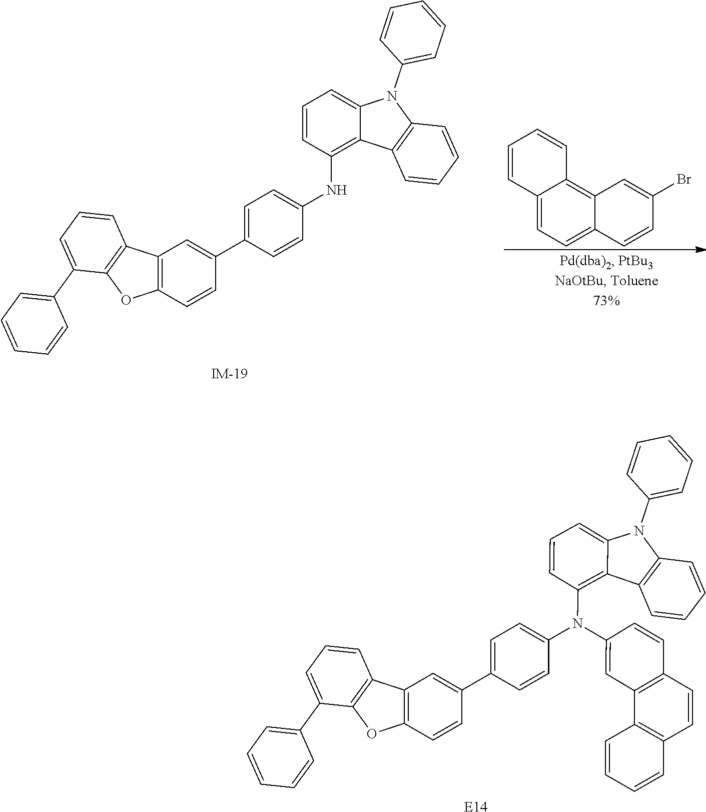

In an Ar atmosphere, in a 300 mL three-neck flask, Intermediate IM-19 (10.00 g, 17.3 mmol), Pd(dba)₂ (0.30 g, 0.03 equiv, 0.5 mmol), NaOtBu (3.33 g, 2.0 equiv, 34.7 mmol), toluene (87 mL), 3-bromophenanthrene (4.90 g, 1.1 equiv, 19.1 mmol), and PtBu₃ (0.35 g, 0.1 equiv, 1.7 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound E14 (9.53 g, yield 73%).

By measuring FAB-MS, a mass number of m/z=752 was observed by molecular ion peak, thereby identifying Compound E14.

### (18) Synthesis of Compound E164

Amine Compound E164 may be synthesized by, for example, the reaction below.

### Synthesis of Intermediate IM-20

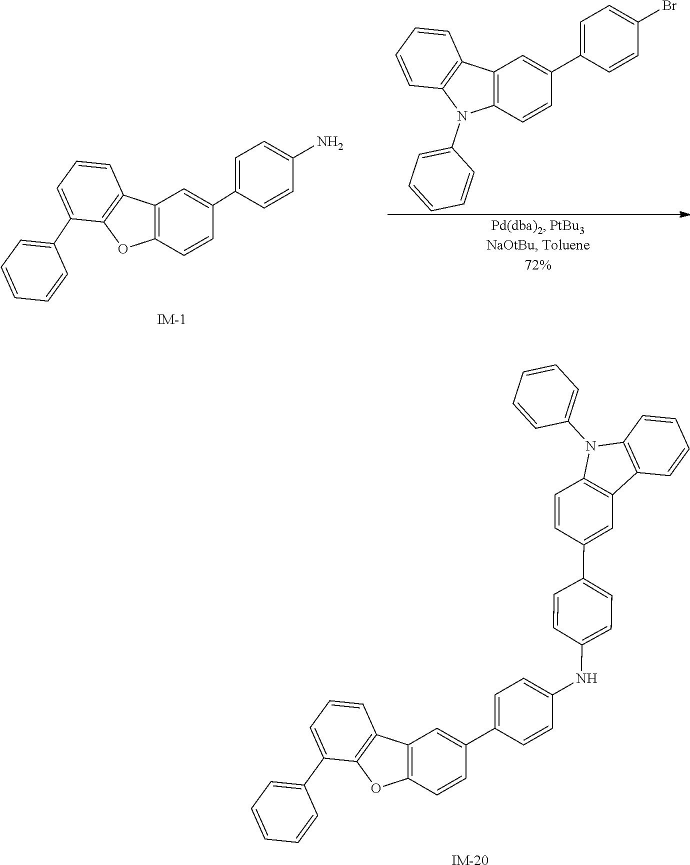

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 3-(4-bromophenyl)-9-phenyl-9H-carbazole (19.59 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-20 (21.02 g, yield 72%).

By measuring FAB-MS, a mass number of m/z=576 was observed by molecular ion peak, thereby identifying Intermediate IM-20.

### Synthesis of Compound E164

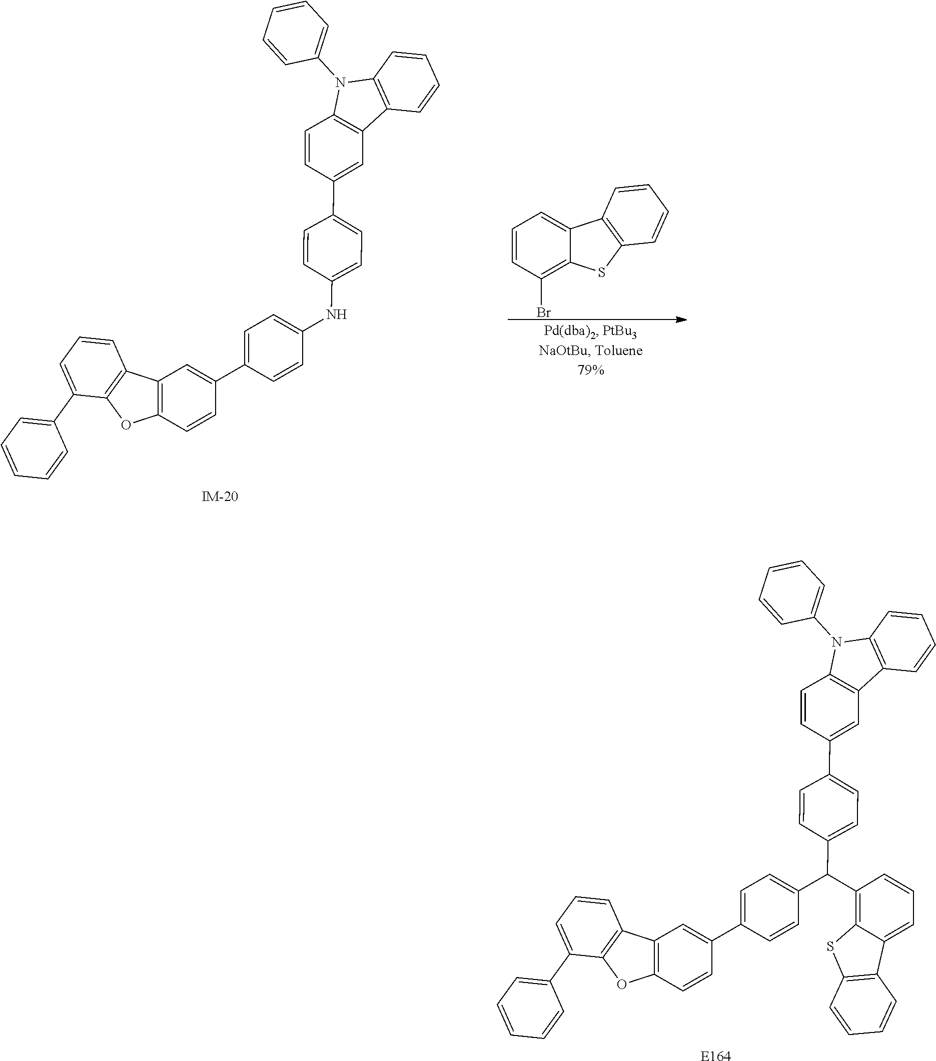

In an Ar atmosphere, in a 300 mL three-neck flask, Intermediate IM-20 (10.00 g, 15.3 mmol), Pd(dba)₂ (0.26 g, 0.03 equiv, 0.5 mmol), NaOtBu (2.94 g, 2.0 equiv, 30.6 mmol), toluene (77 mL), 4-bromodibenzothiophene (4.43 g, 1.1 equiv, 16.9 mmol), and PtBu₃ (0.31 g, 0.1 equiv, 1.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound E164 (10.11 g, yield 79%).

By measuring FAB-MS, a mass number of m/z=835 was observed by molecular ion peak, thereby identifying Compound E164.

### (19) Synthesis of Compound E193

Amine Compound E193 may be synthesized by, for example, the reaction below.

### Synthesis of Intermediate IM-21

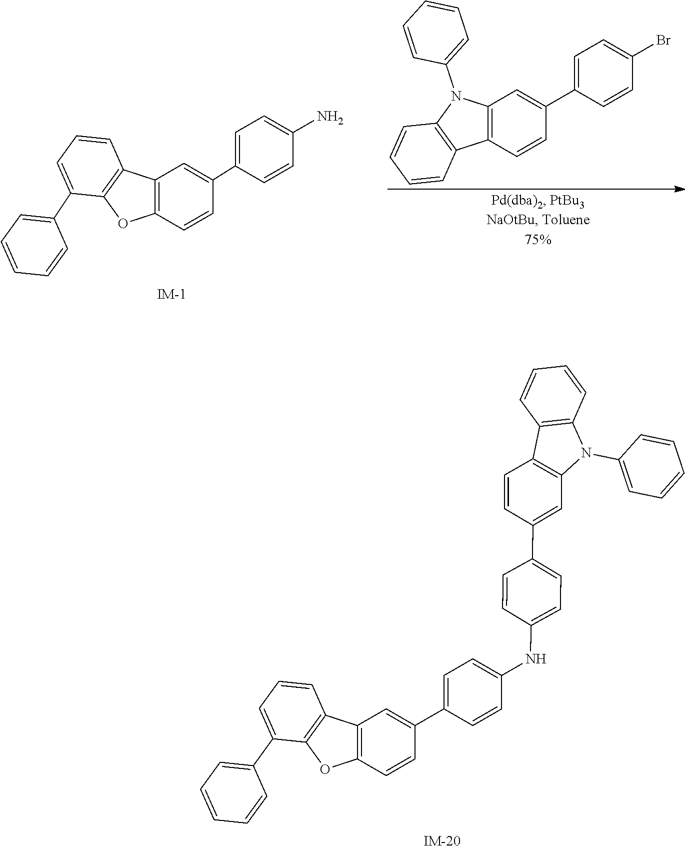

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 2-(4-bromophenyl)-9-phenyl-9H-carbazole (19.59 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-21 (21.90 g, yield 75%).

By measuring FAB-MS, a mass number of m/z=576 was observed by molecular ion peak, thereby identifying Intermediate IM-21.

### Synthesis of Compound E193

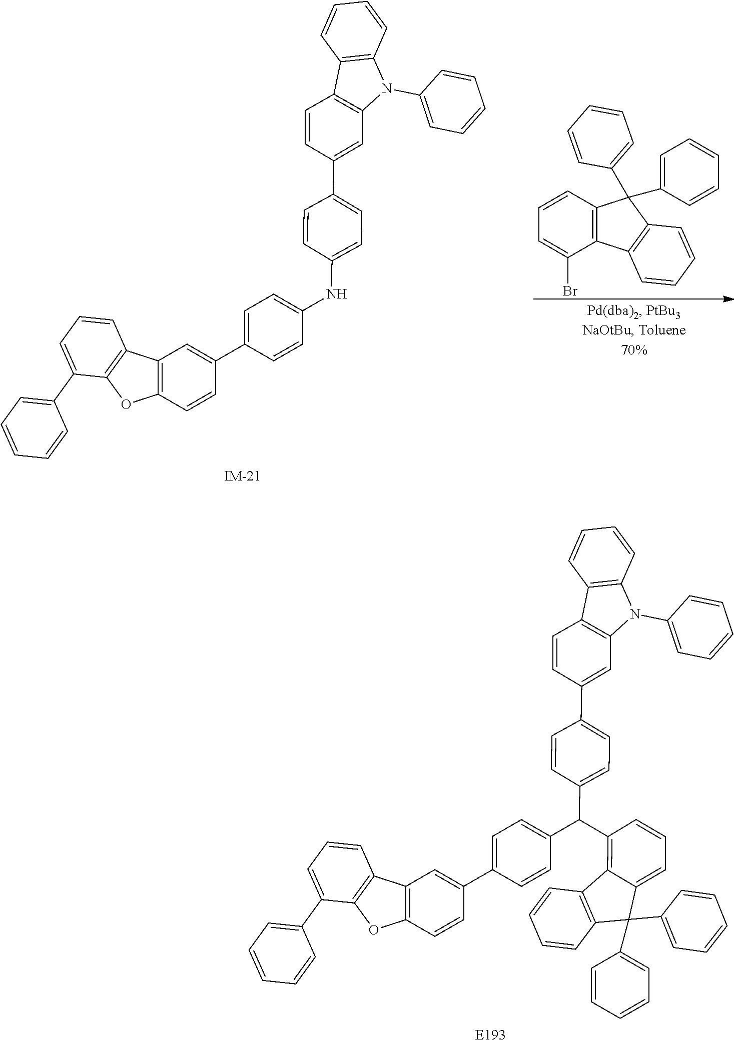

In an Ar atmosphere, in a 300 mL three-neck flask, Intermediate IM-21 (10.00 g, 15.3 mmol), Pd(dba)₂ (0.26 g, 0.03 equiv, 0.5 mmol), NaOtBu (2.94 g, 2.0 equiv, 30.6 mmol), toluene (77 mL), 4-bromo-9,9-diphenyl-9H-fluorene (6.71 g, 1.1 equiv, 16.9 mmol), and PtBu₃ (0.31 g, 0.1 equiv, 1.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound E193 (10.39 g, yield 70%).

By measuring FAB-MS, a mass number of m/z=969 was observed by molecular ion peak, thereby identifying Compound E193.

### (20) Synthesis of Compound E249

Amine Compound E249 may be synthesized by, for example, the reaction below.

### Synthesis of Intermediate IM-22

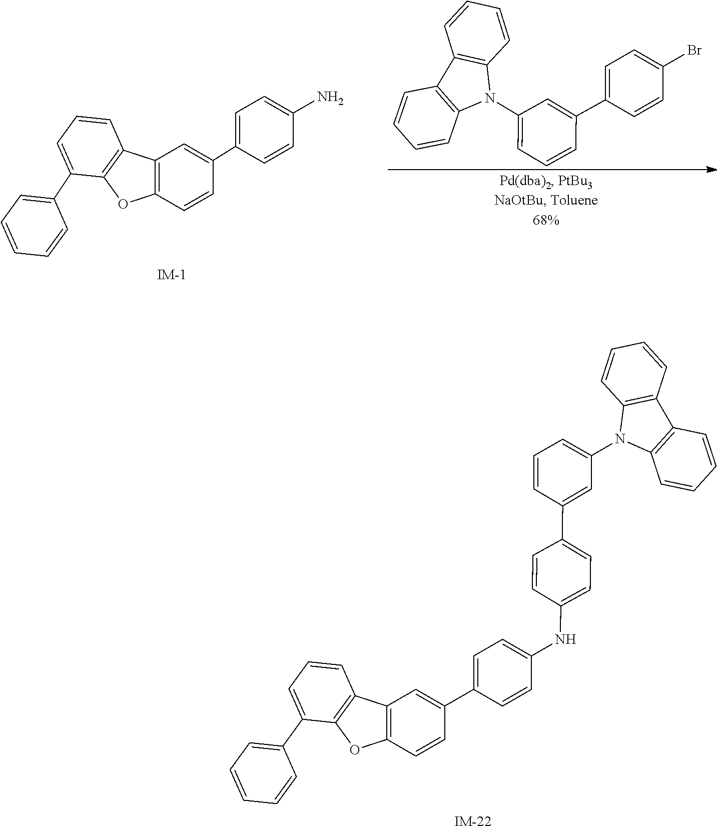

In an Ar atmosphere, in a 500 mL three-neck flask, Intermediate IM-1 (15.00 g, 44.7 mmol), Pd(dba)₂ (0.77 g, 0.03 equiv, 1.3 mmol), NaOtBu (4.30 g, 1.0 equiv, 44.7 mmol), toluene (223 mL), 9-(4′-bromo-[1,1′-biphenyl]-3-yl)-9H-carbazole (19.59 g, 1.1 equiv, 49.2 mmol), and PtBu₃ (0.90 g, 0.1 equiv, 4.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain Intermediate IM-22 (19.85 g, yield 68%).

By measuring FAB-MS, a mass number of m/z=576 was observed by molecular ion peak, thereby identifying Intermediate IM-22.

### Synthesis of Compound E249

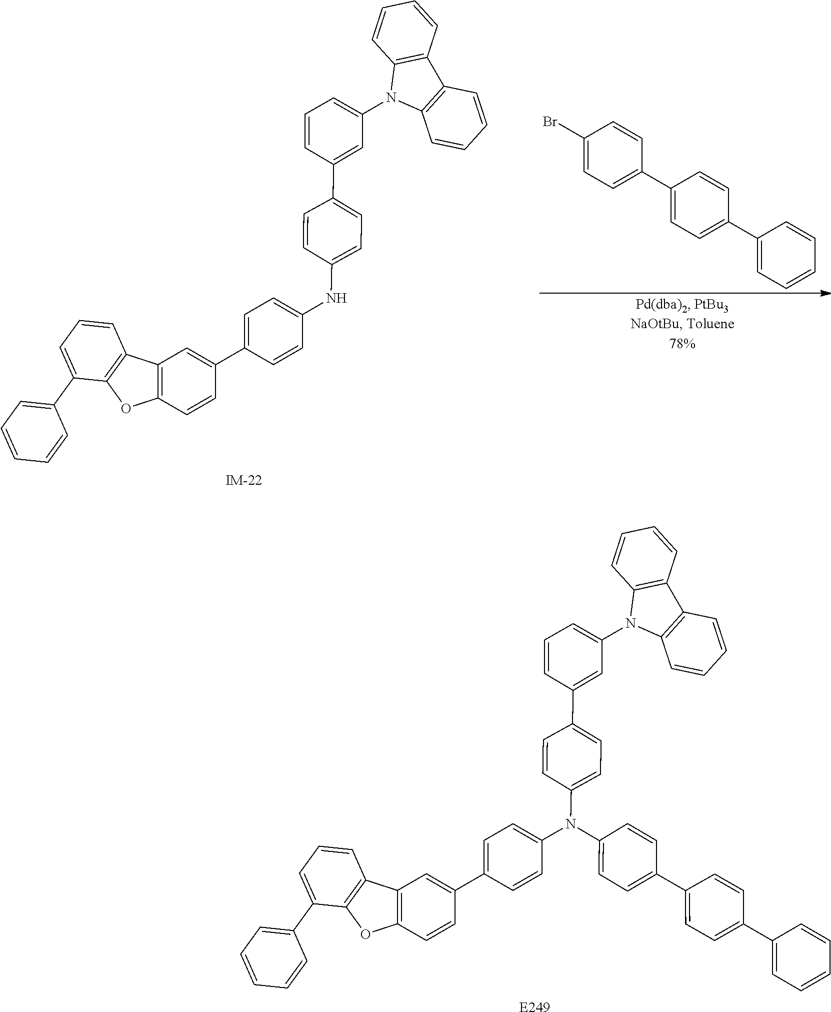

In an Ar atmosphere, in a 300 mL three-neck flask, Intermediate IM-22 (10.00 g, 15.3 mmol), Pd(dba)₂ (0.26 g, 0.03 equiv, 0.5 mmol), NaOtBu (2.94 g, 2.0 equiv, 30.6 mmol), toluene (77 mL), p-bromoterphenyl (5.21 g, 1.1 equiv, 16.9 mmol), and PtBu₃ (0.31 g, 0.1 equiv, 1.5 mmol) were sequentially added, and then heated and stirred under reflux. After the reaction solution was air-cooled to room temperature, organic layers were fractionated by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (using a mixed solvent of hexane and toluene as an eluent) to obtain solid Compound E249 (10.53 g, yield 78%).

By measuring FAB-MS, a mass number of m/z=881 was observed by molecular ion peak, thereby identifying Compound E249.

### 2. Manufacture and Evaluation of Light Emitting Device Including Amine Compound

Evaluation of the light emitting devices including compounds of Examples and Comparative Examples in a hole transport layer was performed as follows. The method for manufacturing the light emitting device for the evaluation of the element is described below.

### (1) Manufacture of Light Emitting Device 1 Using Amine Compound as Hole Transport Layer Material

The light emitting device 1 of an example including the amine compound of an example in a hole transport layer was manufactured as follows. Examples 1-1 to 1-20 correspond to the light emitting devices manufactured by using Compounds A10, A127, A179, A199, B32, B135, B151, B182, C35, C52, C98, C341, C361, D9, D53, D68, E14, E164, E193, and E249 which are Example Compounds as described above as a hole transport layer material, respectively. Comparative Examples 1-1 to 1-15 correspond to the light emitting devices manufactured by using Comparative Example Compounds R1 to R15 as a hole transport layer material, respectively.

ITO was used to form a 150 nm-thick first electrode, 4,4′,4″-tris[N(2-naphthyl)-N-phenylamino]-triphenylamine (2-TNATA) was used to form a 60 nm-thick hole injection layer, Example Compound or Comparative Example Compound was used to form a 30 nm-thick hole transport layer, 2,5,8,11-tetra-t-butylperylene (TBP) was doped by 3% to 9,10-di(naphthalene-2-yl)anthracene (ADN) to form a 25 nm-thick emission layer, tris(8-hydroxyquinolinato)aluminum (Alq₃) was used to form a 25 nm-thick electron transport layer, LiF was used to form a 1 nm-thick electron injection layer, and Al was used to form a 100 nm-thick second electrode. Each layer was formed by a deposition method in a vacuum atmosphere.

### (2) Manufacture of Light Emitting Device 2 Using Amine Compound as Electron Blocking Layer Material

The light emitting device 2 of an example including the amine compound of an example in an electron blocking layer was manufactured as follows. Examples 2-1 to 2-20 correspond to the light emitting devices manufactured by using Compounds A10, A127, A179, A199, B32, B135, B151, B182, C35, C52, C98, C341, C361, D9, D53, D68, E14, E164, E193, and E249 which are Example Compounds as described above as an electron blocking layer material, respectively. Comparative Examples 2-1 to 2-15 correspond to the light emitting devices manufactured by using Comparative Example Compounds R1 to R15 as an electron blocking layer material, respectively.

ITO was used to form a 150 nm-thick first electrode, 4,4′,4″-tris[N(2-naphthyl)-N-phenylamino]-triphenylamine (2-TNATA) was used to form a 60 nm-thick hole injection layer, H-1-1 was used to form a 20 nm-thick hole transport layer, Example Compound or Comparative Example Compound was used to form a 10 nm-thick electron blocking layer, 2,5,8,11-tetra-t-butylperylene (TBP) was doped by 3% to 9,10-di(naphthalene-2-yl)anthracene (ADN) to form a 25 nm-thick emission layer, tris(8-hydroxyquinolinato)aluminum (Alq₃) was used to form a 25 nm-thick electron transport layer, LiF was used to form a 1 nm-thick electron injection layer, and A₁ was used to form a 100 nm-thick second electrode. Each layer was formed by a deposition method in a vacuum atmosphere.

Example Compounds and Comparative Example Compounds used to manufacture light emitting device 1 and light emitting device 2 are as follows:

### Example Compounds

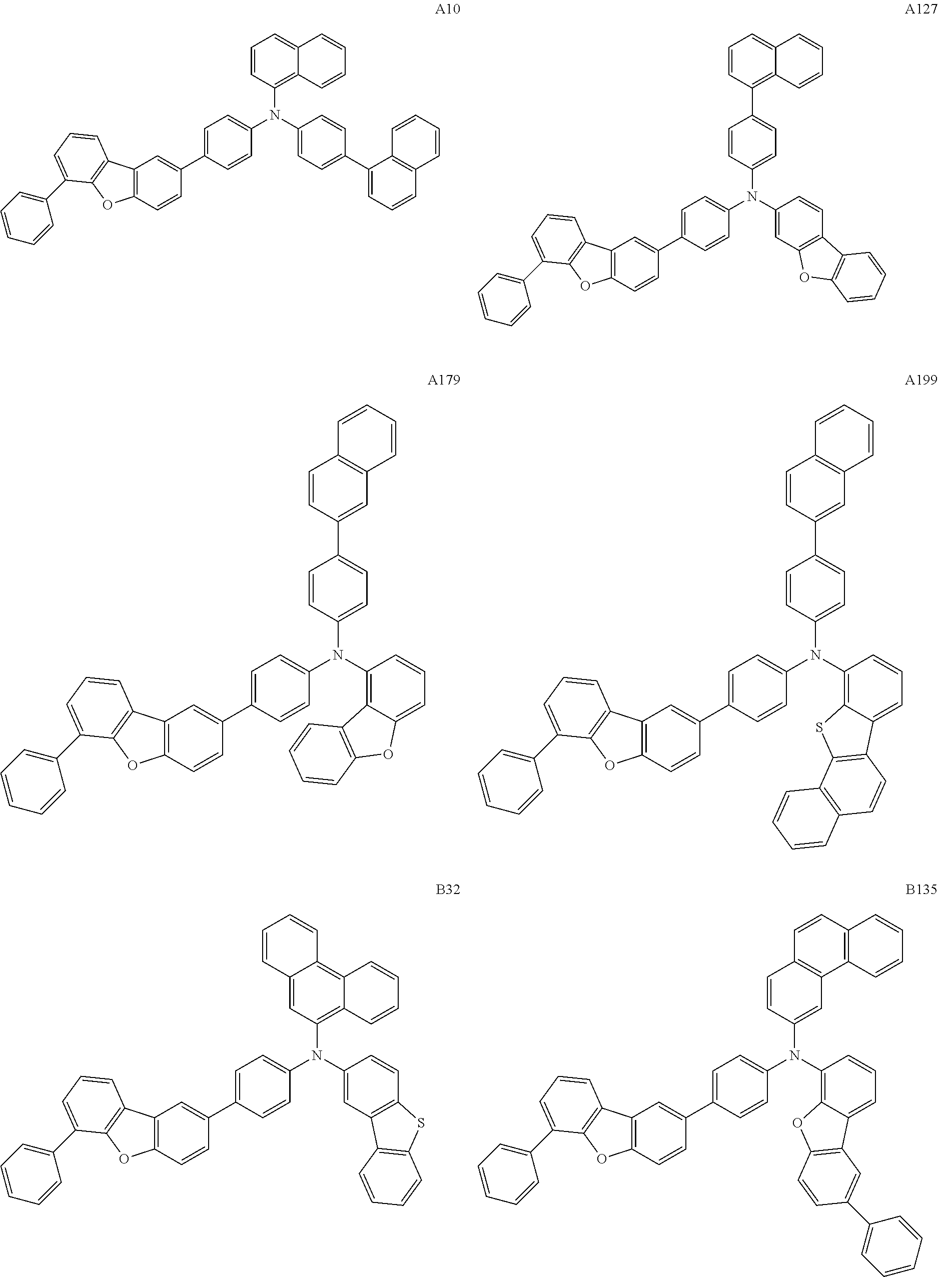

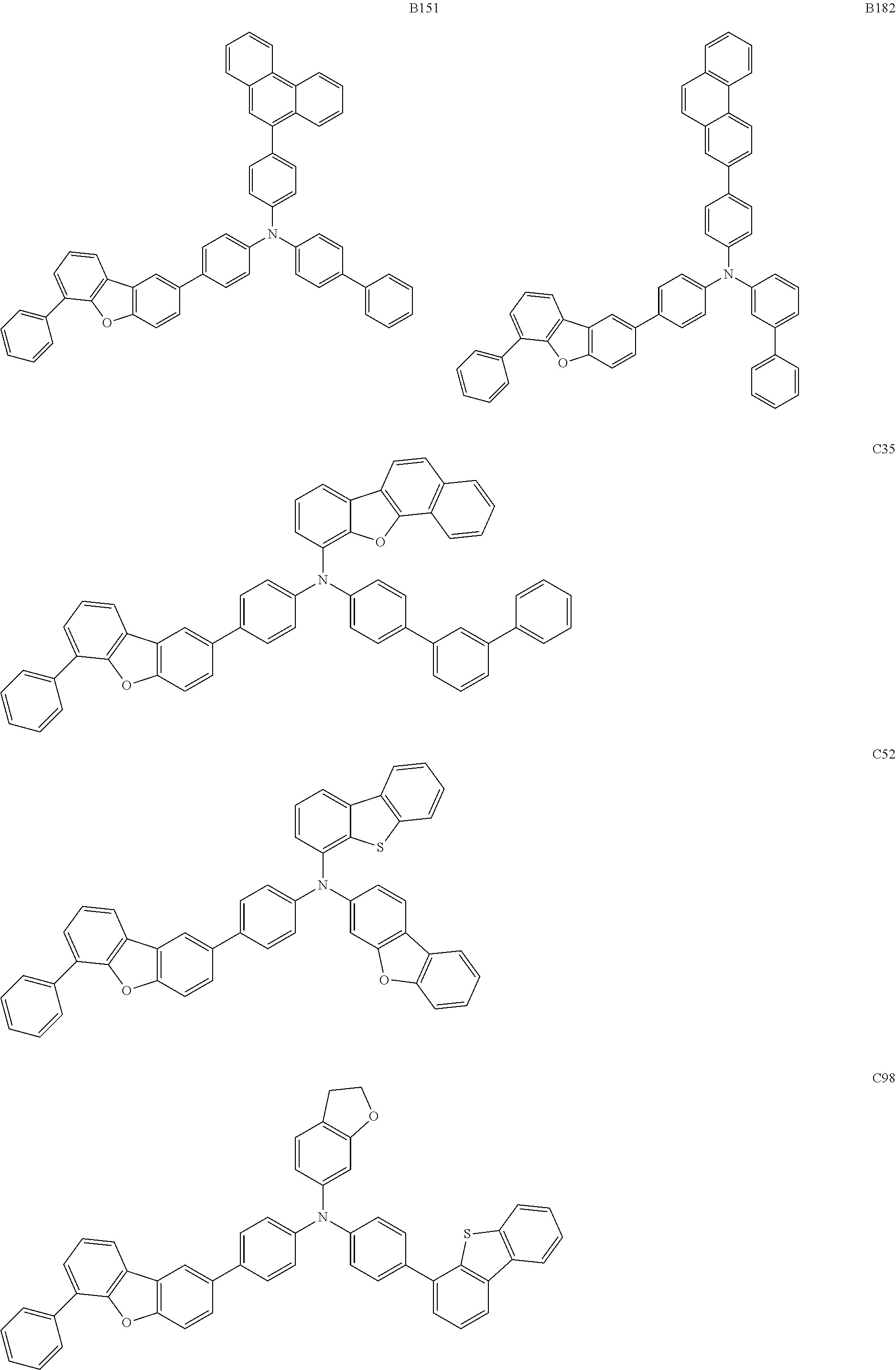 [SMILES: c1ccc(-c2ccc(N(c3ccc(-c4ccc5oc6c(-c7ccccc7)cccc6c5c4)cc3)c3ccc(-c4cc5ccccc5c5ccccc45)cc3)cc2)cc1 ; c1ccc(-c2cccc(N(c3ccc(-c4ccc5c(ccc6ccccc65)c4)cc3)c3ccc(-c4ccc5oc6c(-c7ccccc7)cccc6c5c4)cc3)c2)cc1 ; c1ccc(-c2cccc(-c3ccc(N(c4ccc(-c5ccc6oc7c(-c8ccccc8)cccc7c6c5)cc4)c4cccc5c4oc4c6ccccc6ccc54)cc3)c2)cc1 ; c1ccc(-c2cccc3c2oc2ccc(-c4ccc(N(c5ccc6c(c5)oc5ccccc56)c5cccc6c5sc5ccccc56)cc4)cc23)cc1 ; c1ccc(-c2cccc3c2oc2ccc(-c4ccc(N(c5ccc(-c6cccc7c6sc6ccccc67)cc5)c5ccc6c(c5)OCC6)cc4)cc23)cc1]

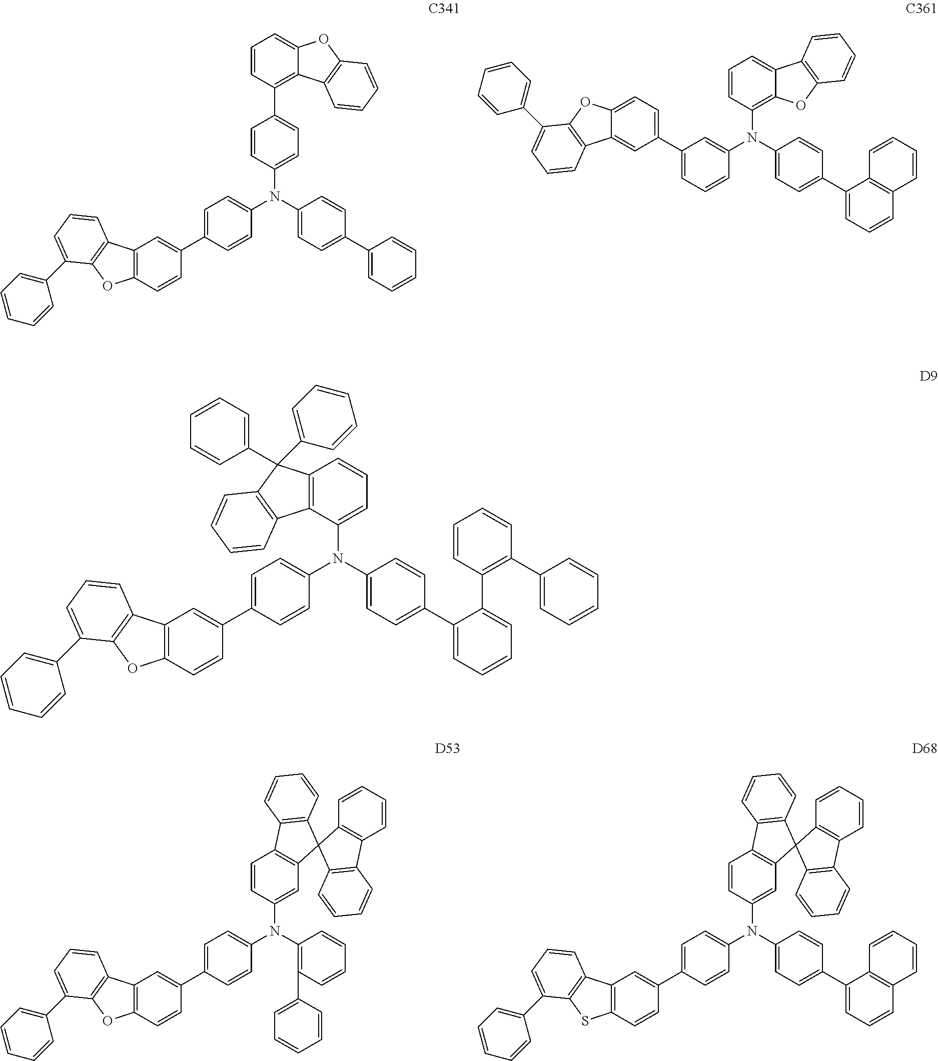

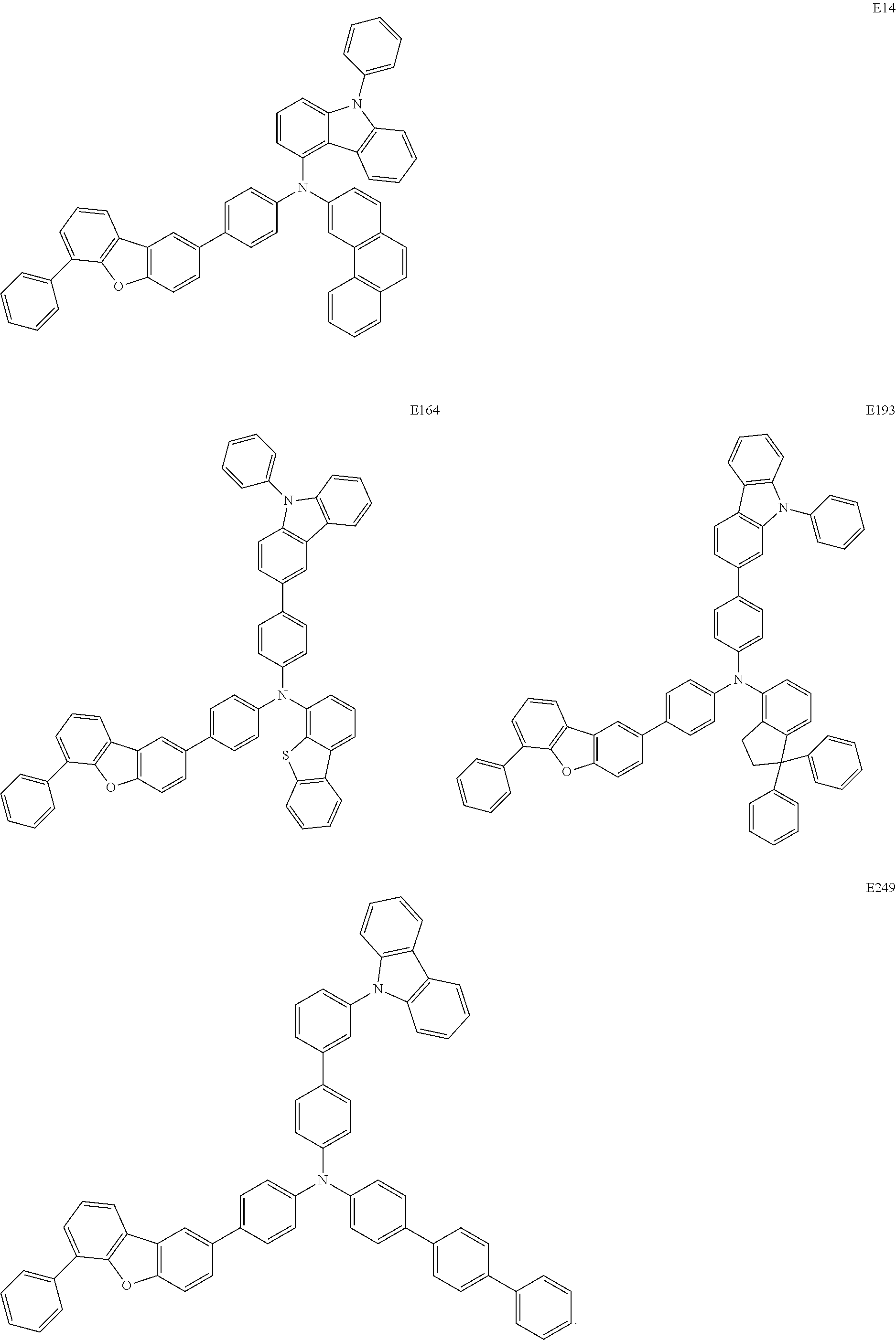 [SMILES: c1ccc(-c2cccc3c2oc2ccc(-c4ccc(N(c5ccc6ccc7ccccc7c6c5)c5cccc6c5c5ccccc5n6-c5ccccc5)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6c5sc5ccccc56)cc4)cc23)cc1 ; c1ccc(-c2cccc3c2oc2ccc(-c4ccc(N(c5ccc(-c6ccc7c8ccccc8n(-c8ccccc8)c7c6)cc5)c5cccc6c5CCC6(c5ccccc5)c5ccccc5)cc4)cc23)cc1 ; c1ccc(-c2ccc(-c3ccc(N(c4ccc(-c5cccc(-n6c7ccccc7c7ccccc76)c5)cc4)c4ccc(-c5ccc6oc7c(-c8ccccc8)cccc7c6c5)cc4)cc3)cc2)cc1 ; "."]

Comparative Example Compounds R1 to R15 were used to manufacture devices of Comparative Examples.

### Comparative Example Compounds

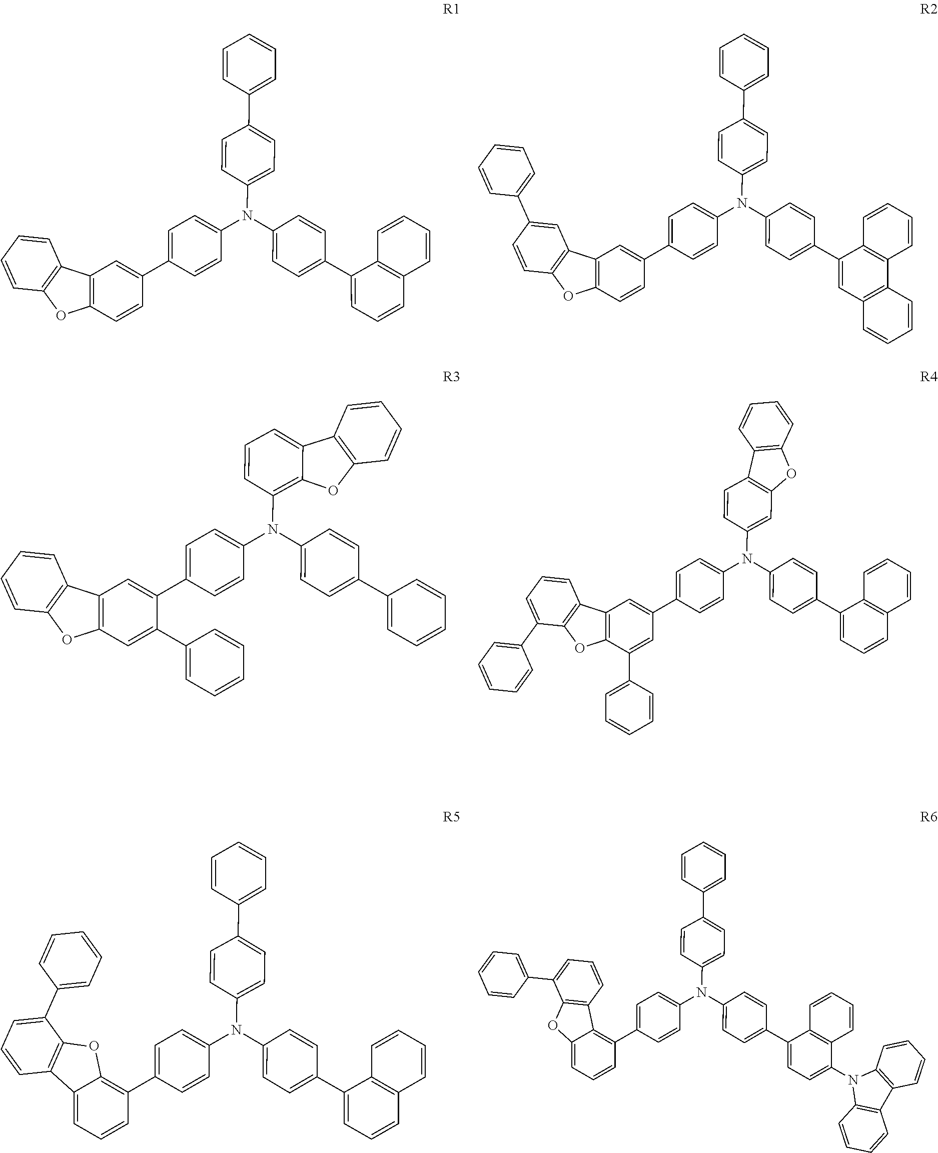

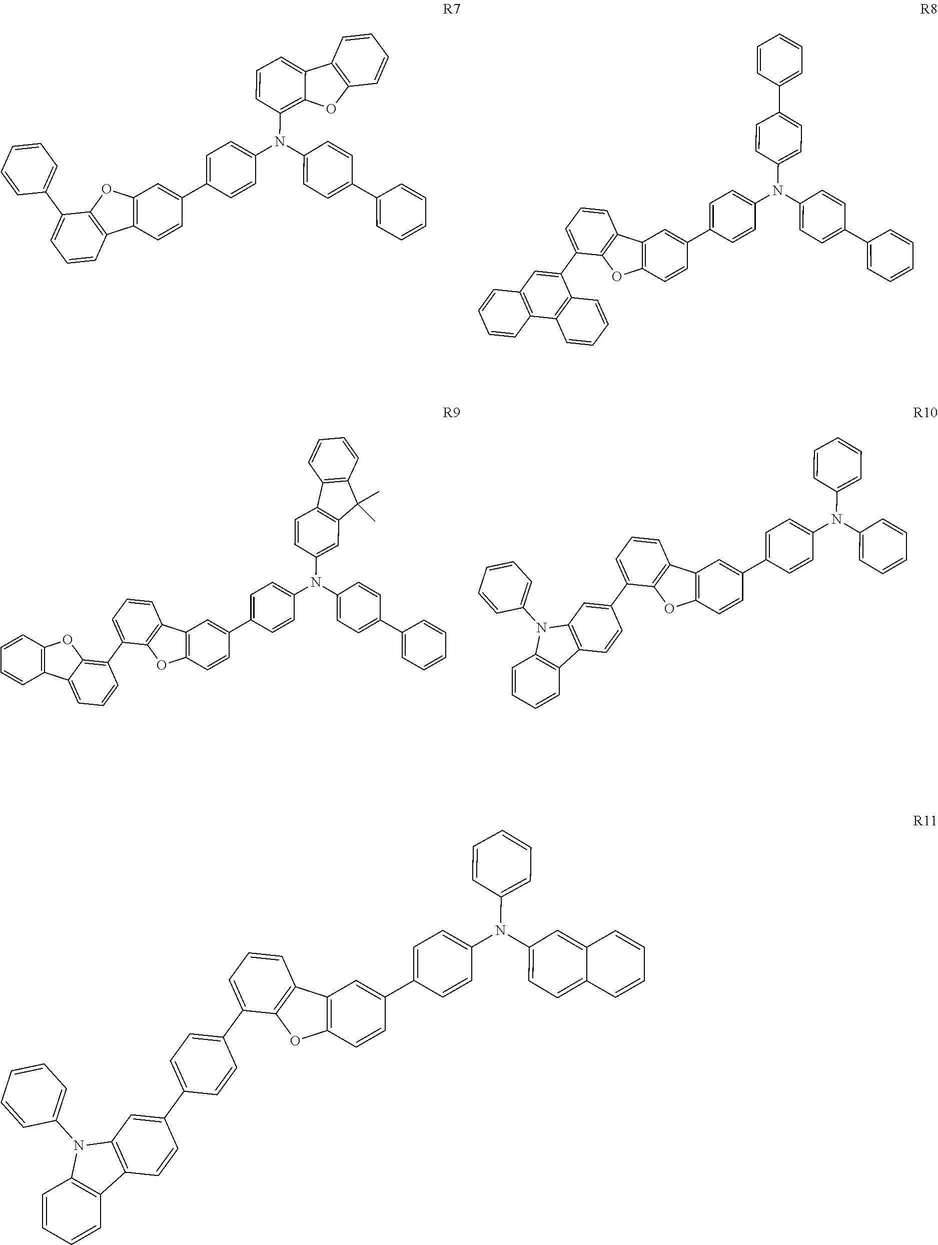 [SMILES: c1ccc(-c2ccc(N(c3ccc(-c4ccc5c(c4)oc4c(-c6ccccc6)cccc45)cc3)c3cccc4c3oc3ccccc34)cc2)cc1 ; c1ccc(-c2ccc(N(c3ccc(-c4ccccc4)cc3)c3ccc(-c4ccc5oc6c(-c7cc8ccccc8c8ccccc78)cccc6c5c4)cc3)cc2)cc1 ; CC1(C)c2ccccc2-c2ccc(N(c3ccc(-c4ccccc4)cc3)c3ccc(-c4ccc5oc6c(-c7cccc8c7oc7ccccc78)cccc6c5c4)cc3)cc21 ; c1ccc(N(c2ccccc2)c2ccc(-c3ccc4oc5c(-c6ccc7c8ccccc8n(-c8ccccc8)c7c6)cccc5c4c3)cc2)cc1 ; c1ccc(N(c2ccc(-c3ccc4oc5c(-c6ccc(-c7ccc8c9ccccc9n(-c9ccccc9)c8c7)cc6)cccc5c4c3)cc2)c2ccc3ccccc3c2)cc1]

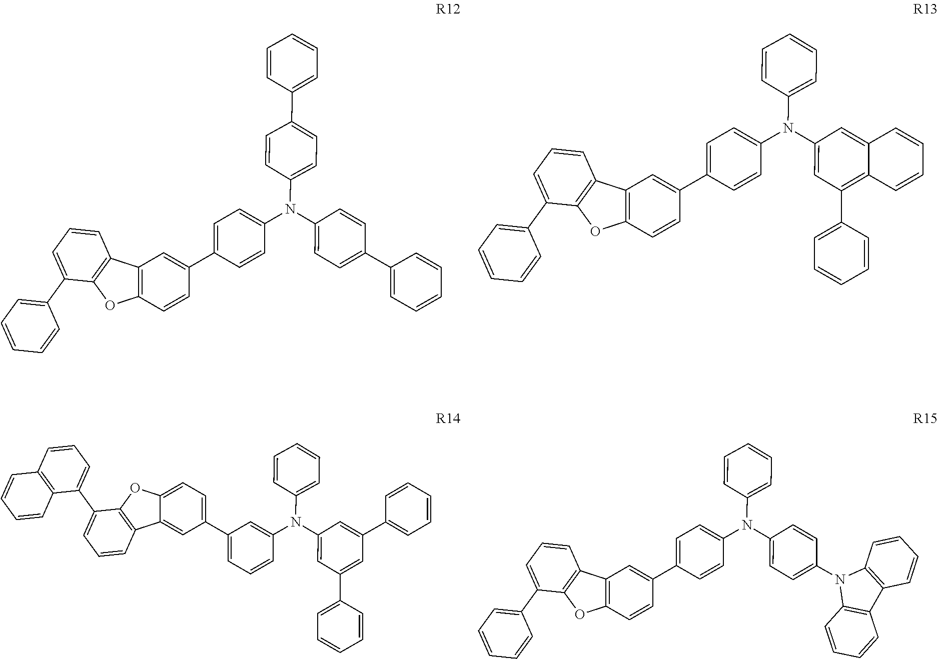 [SMILES: c1ccc(-c2ccc(N(c3ccc(-c4ccccc4)cc3)c3ccc(-c4ccc5oc6c(-c7ccccc7)cccc6c5c4)cc3)cc2)cc1 ; c1ccc(-c2cc(N(c3ccccc3)c3ccc(-c4ccc5oc6c(-c7ccccc7)cccc6c5c4)cc3)cc3ccccc23)cc1 ; c1ccc(-c2cc(-c3ccccc3)cc(N(c3ccccc3)c3cccc(-c4ccc5oc6c(-c7cccc8ccccc78)cccc6c5c4)c3)c2)cc1 ; c1ccc(-c2cccc3c2oc2ccc(-c4ccc(N(c5ccccc5)c5ccc(-n6c7ccccc7c7ccccc76)cc5)cc4)cc23)cc1]

Compounds used for manufacturing the light emitting devices of Examples and Comparative Examples are disclosed below. The compounds below are generally used materials, and commercial products were subjected to sublimation purification and used to manufacture the devices.

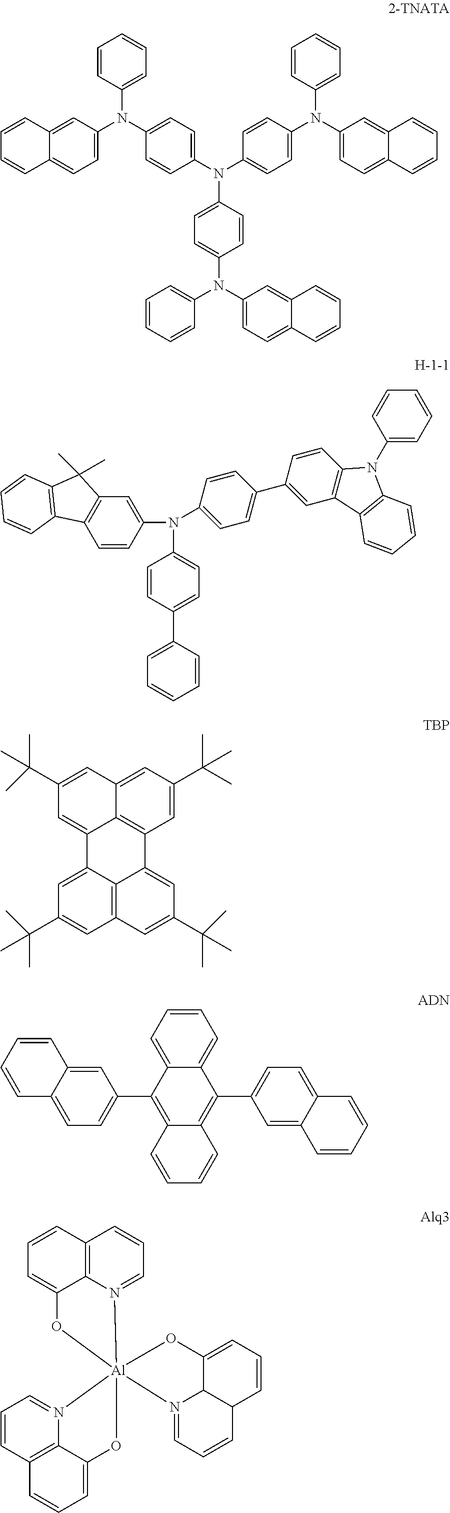

### (3) Evaluation of Light Emitting Device 1 and Light Emitting Device 2

The device efficiency and device service life of the light emitting devices manufactured by using Experimental Example Compounds A10, A127, A179, A199, B32, B135, B151, B182, C35, C52, C98, C341, C361, D9, D53, D68, E14, E164, E193, and E249 and Comparative Example Compounds R1 to R15 as described above were evaluated. Evaluation results of the light emitting device 1 of Examples 1-1 to 1-20 and Comparative Examples 1-1 to 1-15 are listed in Table 1. Evaluation results of the light emitting device 2 of Examples 2-1 to 2-20 Comparative Examples 2-1 to 2-15 are listed in Table 2. The luminous efficiencies and device service lives of the manufactured light emitting devices are listed in comparison in Tables 1 and 2. In the evaluation results of the characteristics for Examples and Comparative Examples listed in Tables 1 and 2, the luminous efficiency shows an efficiency value at a current density of 10 mA/cm2, and the device service life (LT50) shows a brightness half-life at 1.0 mA/cm². In addition, luminous efficiencies and device service lives are represented as a comparative value when efficiency and device service life of Comparative Example 1-1 are considered 100%.

Current densities, voltages and luminous efficiencies of the devices were measured in a dark room by using 2400 Series Source Meter from Keithley Instruments, Inc., CS-200, Color and Luminance Meter from Konica Minolta, Inc., and PC Program LabVIEW 8.2 for the measurement from Japan National Instrument, Inc.

TABLE 1

| Examples of | | | Device |
|---|---|---|---|
| manufactured | | Luminous | service life |
| elements | Hole transport layer | efficiency | (LT50) |
| Example 1-1 | Example Compound A10 | 155% | 186% |
| Example 1-2 | Example Compound A127 | 149% | 195% |
| Example 1-3 | Example Compound A179 | 157% | 180% |
| Example 1-4 | Example Compound A199 | 153% | 198% |
| Example 1-5 | Example Compound B32 | 156% | 178% |
| Example 1-6 | Example Compound B135 | 157% | 181% |
| Example 1-7 | Example Compound B151 | 150% | 183% |
| Example 1-8 | Example Compound B182 | 151% | 180% |
| Example 1-9 | Example Compound C35 | 152% | 185% |
| Example 1-10 | Example Compound C52 | 148% | 187% |
| Example 1-11 | Example Compound C98 | 150% | 190% |
| Example 1-12 | Example Compound C341 | 153% | 175% |
| Example 1-13 | Example Compound C361 | 154% | 173% |
| Example 1-14 | Example Compound D9 | 156% | 170% |
| Example 1-15 | Example Compound D53 | 160% | 168% |
| Example 1-16 | Example Compound D68 | 150% | 192% |
| Example 1-17 | Example Compound E14 | 153% | 189% |
| Example 1-18 | Example Compound E164 | 149% | 190% |
| Example 1-19 | Example Compound E193 | 152% | 187% |
| Example 1-20 | Example Compound E249 | 153% | 180% |
| Comparative | Comparative Example | 100% | 100% |
| Example 1-1 | Compound R1 | | |
| Comparative | Comparative Example | 105% | 96% |
| Example 1-2 | Compound R2 | | |
| Comparative | Comparative Example | 107% | 88% |
| Example 1-3 | Compound R3 | | |
| Comparative | Comparative Example | 101% | 75% |
| Example 1-4 | Compound R4 | | |
| Comparative | Comparative Example | 105% | 105% |
| Example 1-5 | Compound R5 | | |
| Comparative | Comparative Example | 110% | 90% |
| Example 1-6 | Compound R6 | | |
| Comparative | Comparative Example | 100% | 110% |
| Example 1-7 | Compound R7 | | |
| Comparative | Comparative Example | 105% | 90% |
| Example 1-8 | Compound R8 | | |
| Comparative | Comparative Example | 104% | 86% |
| Example 1-9 | Compound R9 | | |
| Comparative | Comparative Example | 102% | 84% |
| Example 1-10 | Compound R10 | | |
| Comparative | Comparative Example | 98% | 80% |
| Example 1-11 | Compound R11 | | |
| Comparative | Comparative Example | 113% | 120% |
| Example 1-12 | Compound R12 | | |
| Comparative | Comparative Example | 110% | 105% |
| Example 1-13 | Compound R13 | | |
| Comparative | Comparative Example | 114% | 100% |
| Example 1-14 | Compound R14 | | |
| Comparative | Comparative Example | 113% | 93% |
| Example 1-15 | Compound R15 | | |

Referring to the results of Table 1, it may be confirmed that Examples of the light emitting devices in which the amine compounds according to examples of the present disclosure are used as a hole transport layer material have improved luminous efficiencies and device service lives compared to Comparative Examples. For the amine compound according to the present disclosure, the dibenzoheterole group may be bonded to the amine group at the second carbon position with a linker located therebetween, and thus a HOMO orbital expands, thereby contributing to the improvement in stability of radical or radical cation state, and at the same time (concurrently), the dibenzoheterole ring is oriented so as to facilitate the intermolecular interaction, and thus the hole transport property may be improved. In addition, for the amine compound of an example, a substituted or unsubstituted phenyl group may be introduced as a substituent at the sixth carbon position of the dibenzoheterole skeleton, thereby sterically protecting the heteroatom in the dibenzoheterole skeleton, and thus the stability of materials during the driving may be improved. Moreover, the first substituent that is one substituent selected from among Formula 2-1 to Formula 2-4 may be introduced as a substituent linked to the amine group besides the dibenzoheterole group, thereby improving the electron resistance and exciton resistance of materials. The above-described effects work synergistically, and thus when the amine compound of an example is introduced as a hole transport layer material of the light emitting device, high efficiency and a long service life may be realized.

It may be confirmed that Comparative Example Compound R1 included in Comparative Example 1-1 is a compound in which a phenyl group is not substituted at the dibenzofuran skeleton, the stability during the driving is not sufficient, and thus both the luminous efficiency and the device service life are reduced.

Comparative Example Compound R2 and Comparative Example Compound R3 included in Comparative Example 1-2 and Comparative Example 1-3, respectively, are compounds having a different substitution position of a phenyl group on the dibenzofuran ring, and thus the heteroatom in the dibenzofuran ring cannot be protected, and the stability during the driving lacks, thereby reducing both the luminous efficiency and device service life compared with Examples.

Comparative Example Compound R4 included in Comparative Example 1-4 is a compound in which two phenyl groups are substituted at the dibenzofuran ring, and the deposition temperature of materials is elevated, and thus the deterioration of materials is caused under the high temperature condition, thereby reducing both the luminous efficiency and device service life compared with Examples.

Comparative Example Compound R5 to Comparative Example Compound R7 included in Comparative Example 1-5 to Comparative Example 1-7, respectively, are compounds having a different binding position between the dibenzofuran group and the amine group from Example Compounds, and thus the hole transport property and the expansion of the HOMO orbital are not sufficient compared with Example Compounds, thereby reducing both the luminous efficiency and device service life compared with Examples.

Comparative Example Compound R8 to Comparative Example Compound R10 included in Comparative Example 1-8 to Comparative Example 1-10, respectively, are compounds in which a polycyclic aromatic ring and a heterocyclic group are bonded on the dibenzofuran ring, and the deposition temperature of materials is elevated, and thus the deterioration of materials is caused under the high temperature condition, thereby reducing both the luminous efficiency and device service life compared with Examples.

Comparative Example Compound R11 included in Comparative Example 1-11 is a compound having the dibenzoheterole skeleton like Example Compounds, but both the luminous efficiency and device service life are reduced compared with Examples. Comparative Example Compound R11 does not include, as a substituent linked to the amine group, the first substituent that is one substituent selected from among Formula 2-1 to Formula 2-4 represented in this present disclosure, and it is believed that as a result the electron resistance and exciton resistance are not sufficient.

Comparative Example Compound R12 to Comparative Example Compound R15 included in Comparative Example 1-12 to Comparative Example 1-15 all include the first substituent that is one substituent selected from among Formula 2-1 to Formula 2-4 represented in the present disclosure, but an unsubstituted phenyl group is substituted at the amine group, and thus both the luminous efficiency and device service life are reduced compared with Examples. Comparative Example Compound R12 to Comparative Example Compound R15 have a difference of including the unsubstituted phenyl group compared with the amine compounds of Examples. The amine compounds of Examples of the present disclosure may include two first substituents each linked to the amine group or may include one first substituent and a substituted or unsubstituted aryl group having at least 10 ring-forming carbon atoms, thereby exhibiting an improved hole transport property. For example, the amine compounds of Examples may have improved hole transport property by substituting at least one first substituent, and may have an improvement in electron resistance and exciton resistance by introducing a substituent containing at least a predetermined number of carbons, thereby exhibiting improved luminous efficiency and service life characteristics. In contrast, it may be confirmed that Comparative Example Compound R12 to Comparative Example Compound R15 all have a structure in which the unsubstituted phenyl group is linked to the amine group, and thus the electron resistance and exciton resistance are not sufficient, thereby reducing both the luminous efficiency and device service life compared with Examples.

TABLE 2

| Examples of | | | Device |
|---|---|---|---|
| manufactured | | Luminous | service life |
| elements | Electron blocking layer | efficiency | (LT50) |
| Example 2-1 | Example Compound A10 | 157% | 182% |
| Example 2-2 | Example Compound A127 | 145% | 194% |
| Example 2-3 | Example Compound A179 | 160% | 188% |
| Example 2-4 | Example Compound A199 | 154% | 195% |
| Example 2-5 | Example Compound B32 | 156% | 180% |
| Example 2-6 | Example Compound B135 | 158% | 174% |
| Example 2-7 | Example Compound B151 | 155% | 176% |
| Example 2-8 | Example Compound B182 | 147% | 184% |
| Example 2-9 | Example Compound C35 | 150% | 180% |
| Example 2-10 | Example Compound C52 | 155% | 182% |
| Example 2-11 | Example Compound C98 | 154% | 194% |
| Example 2-12 | Example Compound C341 | 157% | 175% |
| Example 2-13 | Example Compound C361 | 155% | 170% |
| Example 2-14 | Example Compound D9 | 160% | 168% |
| Example 2-15 | Example Compound D53 | 162% | 169% |
| Example 2-16 | Example Compound D68 | 155% | 189% |
| Example 2-17 | Example Compound E14 | 154% | 187% |
| Example 2-18 | Example Compound E164 | 152% | 195% |
| Example 2-19 | Example Compound E193 | 150% | 180% |
| Example 2-20 | Example Compound E249 | 149% | 178% |
| Comparative | Comparative Example | 100% | 100% |
| Example 2-1 | Compound R1 | | |
| Comparative | Comparative Example | 107% | 103% |
| Example 2-2 | Compound R2 | | |
| Comparative | Comparative Example | 108% | 91% |
| Example 2-3 | Compound R3 | | |
| Comparative | Comparative Example | 103% | 79% |
| Example 2-4 | Compound R4 | | |
| Comparative | Comparative Example | 108% | 103% |
| Example 2-5 | Compound R5 | | |
| Comparative | Comparative Example | 111% | 98% |
| Example 2-6 | Compound R6 | | |
| Comparative | Comparative Example | 104% | 106% |
| Example 2-7 | Compound R7 | | |
| Comparative | Comparative Example | 103% | 94% |
| Example 2-8 | Compound R8 | | |
| Comparative | Comparative Example | 107% | 91% |
| Example 2-9 | Compound R9 | | |
| Comparative | Comparative Example | 101% | 87% |
| Example 2-10 | Compound R10 | | |
| Comparative | Comparative Example | 100% | 83% |
| Example 2-11 | Compound R11 | | |
| Comparative | Comparative Example | 116% | 118% |
| Example 2-12 | Compound R12 | | |
| Comparative | Comparative Example | 109% | 107% |
| Example 2-13 | Compound R13 | | |
| Comparative | Comparative Example | 111% | 98% |
| Example 2-14 | Compound R14 | | |
| Comparative | Comparative Example | 109% | 96% |
| Example 2-15 | Compound R15 | | |

Referring to the results of Table 2, it may be confirmed that the light emitting devices of Examples 2-1 to 2-20 exhibit a long service life and high efficiency characteristics compared with those of Comparative Examples 2-1 to 2-15. For example, it may be seen that even when the amine compound of an example is used in the electron blocking layer, the light emitting element may exhibit excellent (suitable) device characteristics.

The light emitting device of an embodiment may exhibit improved device characteristics with high efficiency and a long service life.

The amine compound of an embodiment may be included in the hole transport region of the light emitting device to contribute to high efficiency and a long service life of the light emitting device.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The light emitting device or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one of ordinary skill in the art without departing from the spirit and scope of the present disclosure as defined by the following claims and equivalents thereof.

What is claimed is:

1. A light emitting device comprising:
a first electrode;
a second electrode facing the first electrode; and
a plurality of functional layers between the first electrode and the second electrode,
wherein at least one functional layer among the plurality of functional layers comprises an amine compound represented by Formula 1: 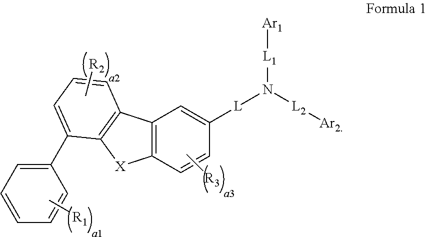
wherein, in Formula 1,
X is O or S,
R₁ to R₃ are each independently a hydrogen atom, or a deuterium atom,
a1 is an integer from 0 to 5,
a2 and a3 are each independently an integer from 0 to 3,
L is a substituted or unsubstituted arylene group having 6 to 30 ring-forming carbon atoms,
L₁ and L₂ are each independently a direct linkage, a substituted or unsubstituted arylene group having 6 to 30 ring-forming carbon atoms, or a substituted or unsubstituted heteroarylene group having 2 to 30 ring-forming carbon atoms,
Ar₁ is represented by any one among Formula 2-1 to Formula 2-4, and
Ar₂ is a substituted or unsubstituted aryl group having 10 to 30 ring-forming carbon atoms, or a substituted or unsubstituted heteroaryl group having 2 to 30 ring-forming carbon atoms, 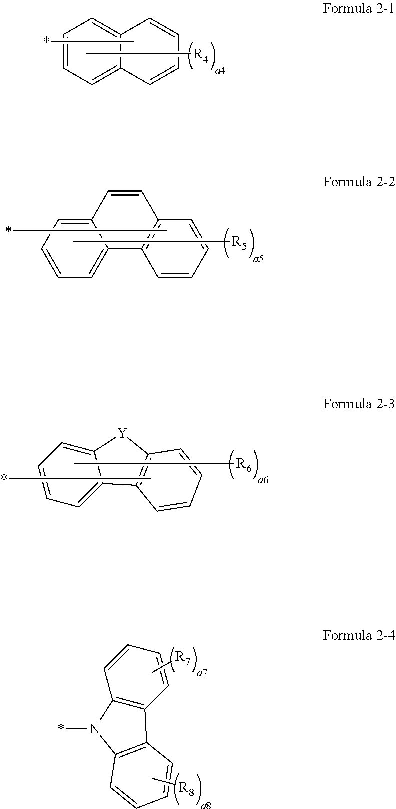
wherein, in Formula 2-1 to Formula 2-4,
R₄ to R₈ are each independently a hydrogen atom, a deuterium atom, a halogen atom, a substituted or unsubstituted alkyl group having 1 to 30 carbon group, a substituted or unsubstituted alkenyl group having 2 to 30 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring,
Y is O, S, NR₉, or CR₁₀R₁₁,
R₉ to R₁₁ are each independently a substituted or unsubstituted alkyl group having 1 to 20 carbon atoms, or a substituted or unsubstituted aryl group having 6 to 30 ring-forming carbon atoms, or are bonded to an adjacent group to form a ring,
a4 and a6 are each independently an integer from 0 to 7,
a5 is an integer from 0 to 9,
a7 and a8 are each independently an integer from 0 to 4, and [symbol]
is a position linked to Formula 1.

2. The light emitting device of claim 1, wherein the plurality of functional layers comprise:
a hole transport region on the first electrode;
an emission layer on the hole transport region; and
an electron transport region on the emission layer,
the hole transport region comprises the amine compound represented by Formula 1.

3. The light emitting device of claim 2, wherein the hole transport region further comprises:
a hole injection layer on the first electrode; and
a hole transport layer on the hole injection layer, and
the hole transport layer comprises the amine compound represented by Formula 1.

4. The light emitting device of claim 2, wherein the hole transport region further comprises:
a hole transport layer on the first electrode; and
an electron blocking layer on the hole transport layer, and
the electron blocking layer comprises the amine compound represented by Formula 1.

5. The light emitting device of claim 1, wherein Ar₂ is represented by any one among Formula 3-1 to Formula 3-3 or any one among Formula 2-1 to Formula 2-4: 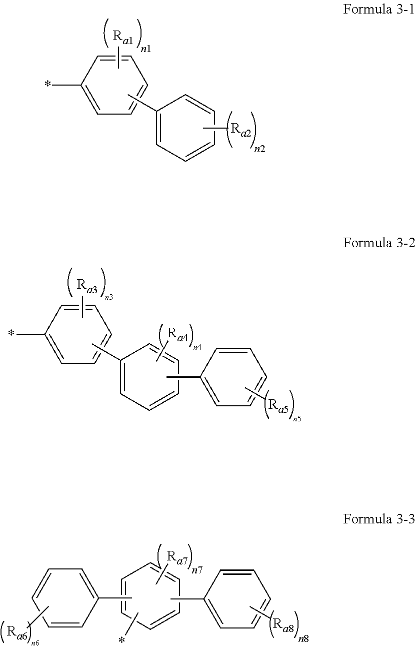
wherein, in Formula 3-1 to Formula 3-3,
Rₐ₁ to Rₐ₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring,
n1, n3 and n4 are each independently an integer from 0 to 4,
n2, n5, n6, and n8 are each independently an integer from 0 to 5,
n7 is an integer from 0 to 3, and [symbol]
is a position linked to Formula 1.

6. The light emitting device of claim 1, wherein Ar₁ is represented by Formula 2-1-1 or Formula 2-1-2: 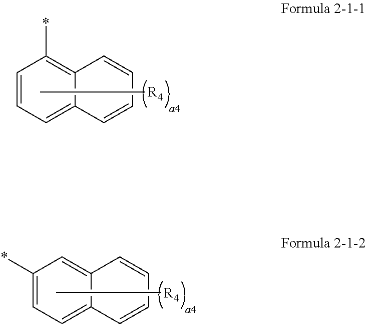
wherein, in Formula 2-1-1 and Formula 2-1-2, R₄ and a4 are the same as defined in Formula 2-1.

7. The light emitting device of claim 1, wherein Ar₁ is represented by any one among Formula 2-2-1 to Formula 2-2-3: 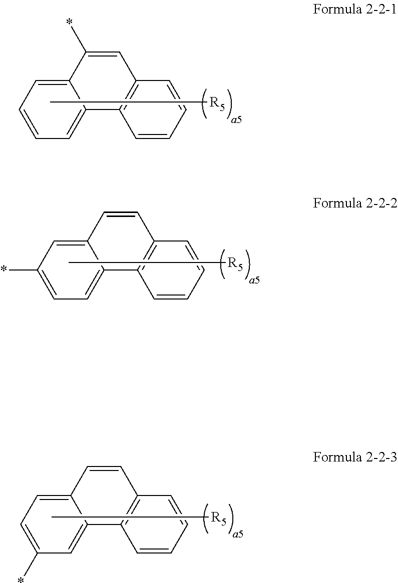
wherein, in Formula 2-2-1 to Formula 2-2-3, R₅ and a5 are the same as defined in Formula 2-2.

8. The light emitting device of claim 1, wherein Ar₁ is represented by any one among Formula 2-3-1 to Formula 2-3-4: 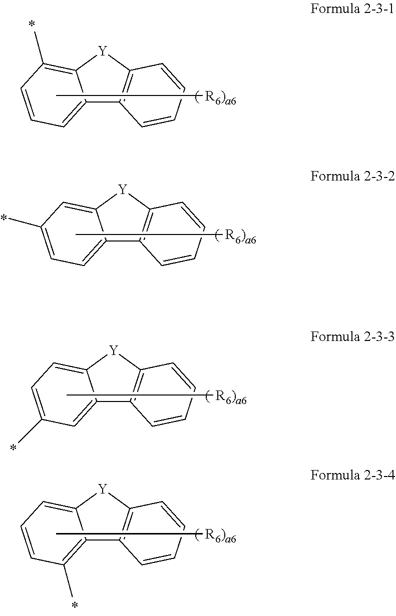
wherein, in Formula 2-3-1 to Formula 2-3-4, Y, R₆ and a6 are the same as defined in Formula 2-3.

9. The light emitting device of claim 1, wherein Ar₁ is represented by any one among Formula 4-1 to Formula 4-7: 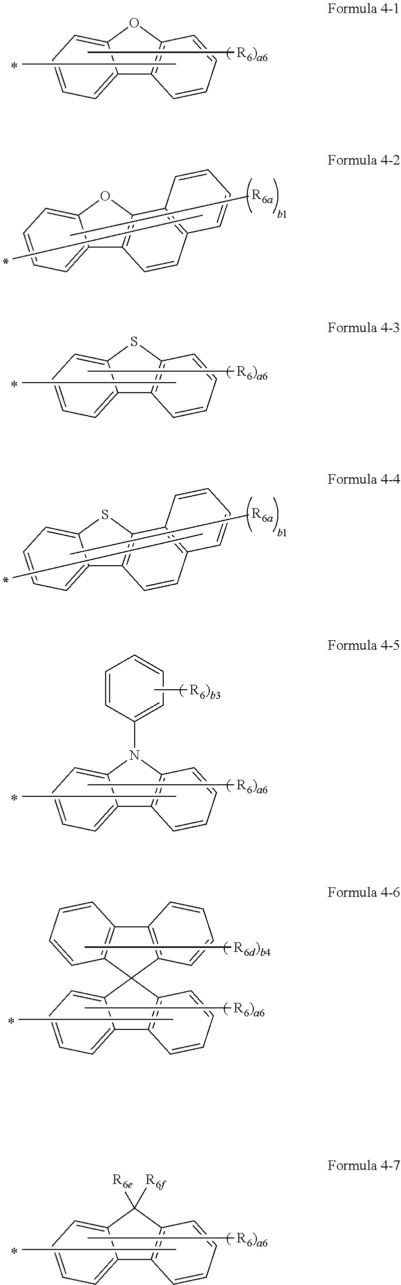
wherein, in Formula 4-1 to Formula 4-7,
R₆ₐ to R_{6d} are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
R₆ₑ and R_{6f} are each independently a substituted or unsubstituted alkyl group having 1 to 20 carbon atoms, or a substituted or unsubstituted aryl group having 6 to 30 ring-forming carbon atoms,
b1 and b2 are each independently an integer from 0 to 9,
b3 is an integer from 0 to 5,
b4 is an integer from 0 to 8, and
R₆ and a6 are the same as defined in Formula 2-3.

10. The light emitting device of claim 1, wherein the amine compound represented by Formula 1 is represented by any one among Formula 5-1 to Formula 5-4: 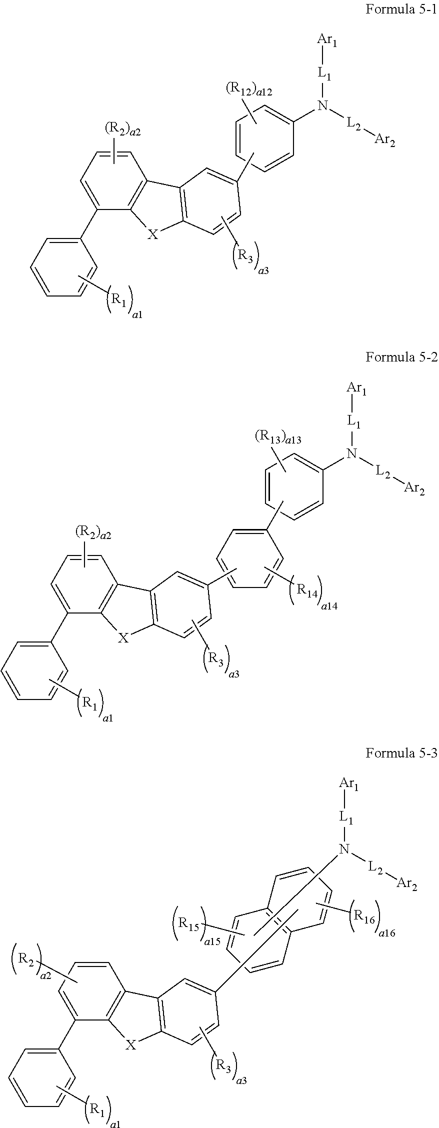 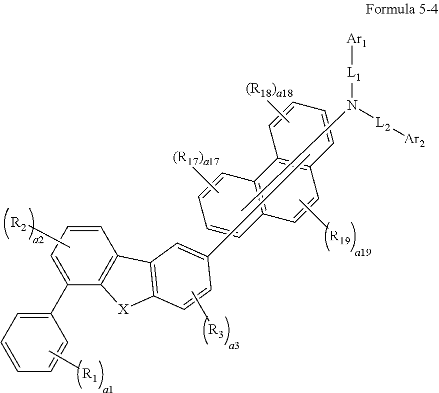
wherein, in Formula 5-1 to Formula 5-4,
R₁₂ to R₁₉ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
a12 to a18 are each independently an integer from 0 to 4,
a19 is an integer from 0 to 2,
the sum of a15 and a16 is 6 or less,
the sum of a17 to a19 is 8 or less, and
R₁ to R₃, a1 to a3, L₁, L₂, X, Ar₁, and Ar₂ are the same as defined in Formula 1.

11. The light emitting device of claim 1, wherein the amine compound represented by Formula 1 is represented by any one among Formula 6-1 to Formula 6-3: 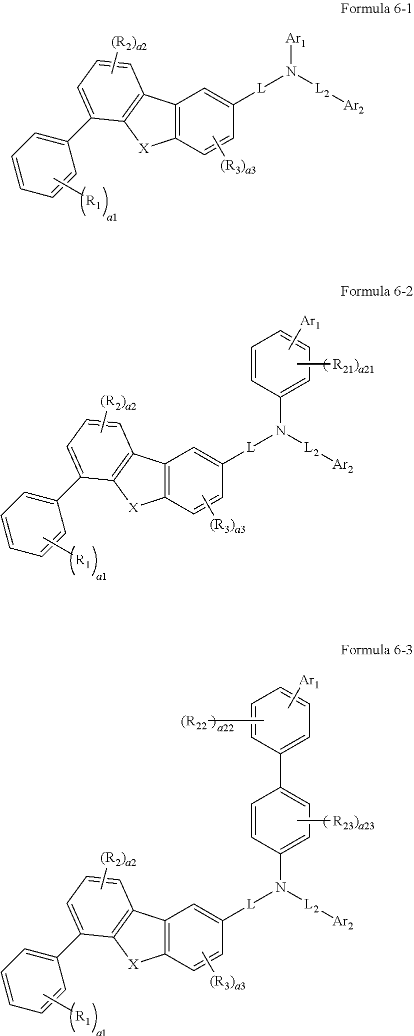
wherein, in Formula 6-1 to Formula 6-3,
R₂₁ to R₂₃ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
a21 to a23 are each independently an integer from 0 to 4, and
R₁ to R₃, a1 to a3, L, L₂, X, Ar₁, and Ar₂ are the same as defined in Formula 1.

12. The light emitting device of claim 1, wherein L is represented by any one among Formula L-1 to Formula L-7: 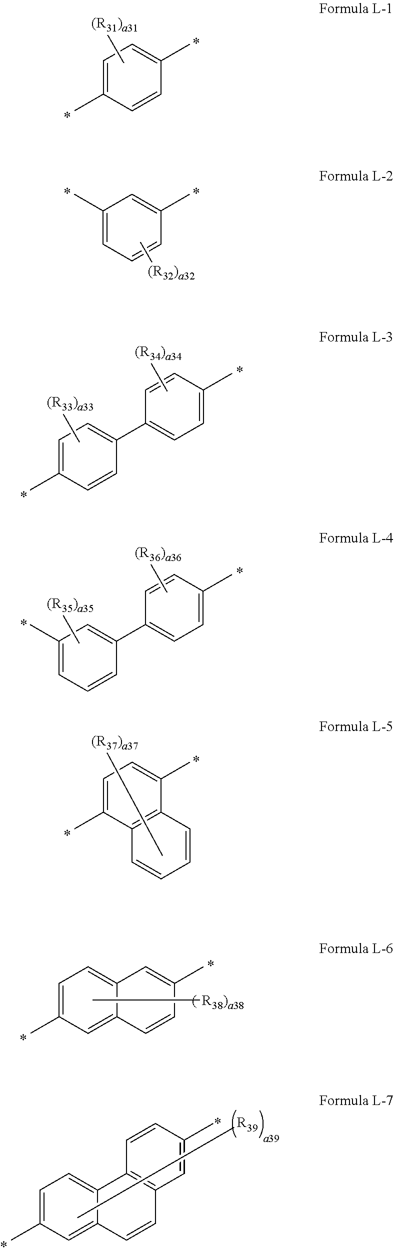
wherein, in Formula L-1 to Formula L-7,
R₃₁ to R₃₉ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
a31 to a36 are each independently an integer from 0 to 4,
a37 and a38 are each independently an integer from 0 to 6, and
a39 is an integer from 0 to 8.

13. The light emitting device of claim 1, wherein the amine compound represented by Formula 1 comprises at least one from among the compounds in Compound Group 1:

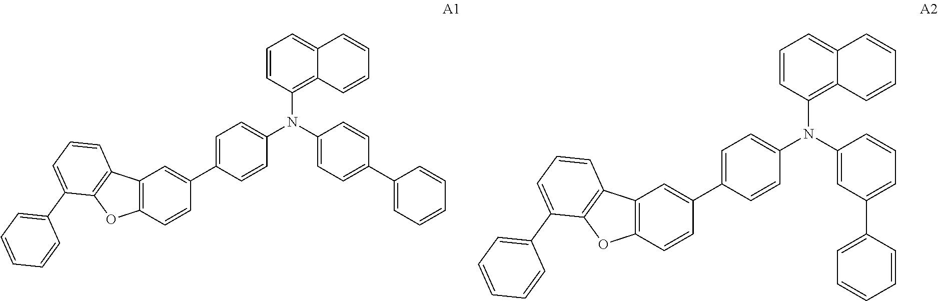 [SMILES: c1ccc(-c2ccc(N(c3ccc(-c4ccc5oc6c(-c7ccccc7)cccc6c5c4)cc3)c3cccc4ccccc34)cc2)cc1 ; c1ccc(-c2cccc(N(c3ccc(-c4ccc5oc6c(-c7ccccc7)cccc6c5c4)cc3)c3cccc4ccccc34)c2)cc1]

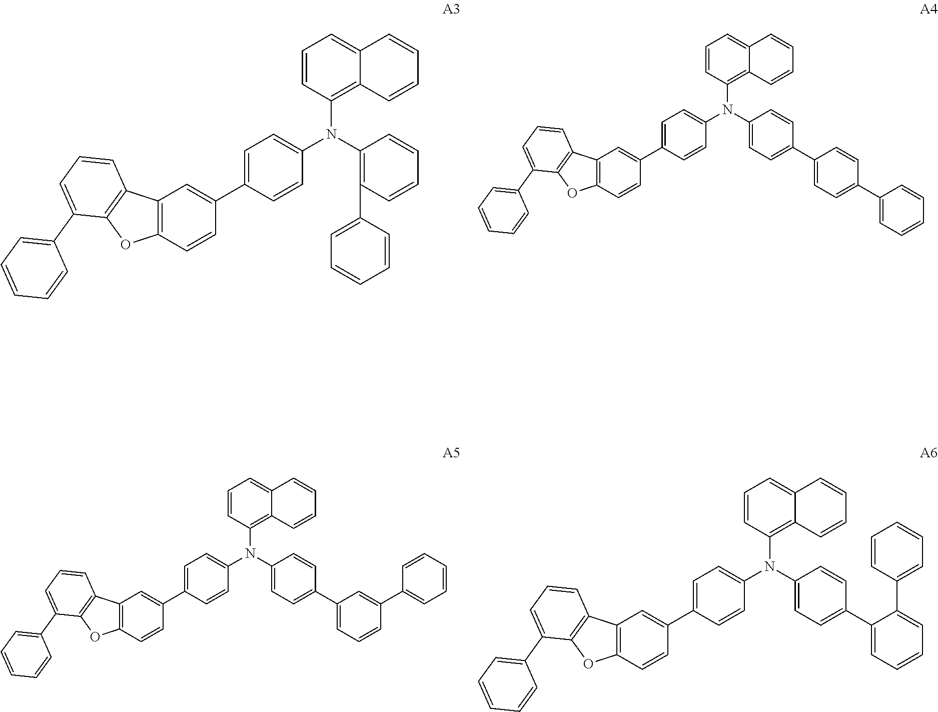 [SMILES: c1ccc(-c2ccccc2N(c2ccc(-c3ccc4oc5c(-c6ccccc6)cccc5c4c3)cc2)c2cccc3ccccc23)cc1 ; c1ccc(-c2ccc(-c3ccc(N(c4ccc(-c5ccc6oc7c(-c8ccccc8)cccc7c6c5)cc4)c4cccc5ccccc45)cc3)cc2)cc1 ; c1ccc(-c2cccc(-c3ccc(N(c4ccc(-c5ccc6oc7c(-c8ccccc8)cccc7c6c5)cc4)c4cccc5ccccc45)cc3)c2)cc1 ; c1ccc(-c2ccccc2-c2ccc(N(c3ccc(-c4ccc5oc6c(-c7ccccc7)cccc6c5c4)cc3)c3cccc4ccccc34)cc2)cc1]

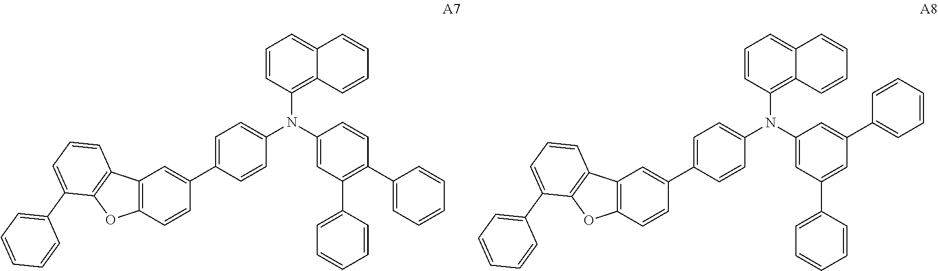 [SMILES: c1ccc(-c2ccc(N(c3ccc(-c4ccc5oc6c(-c7ccccc7)cccc6c5c4)cc3)c3cccc4ccccc34)cc2-c2ccccc2)cc1 ; c1ccc(-c2cc(-c3ccccc3)cc(N(c3ccc(-c4ccc5oc6c(-c7ccccc7)cccc6c5c4)cc3)c3cccc4ccccc34)c2)cc1]

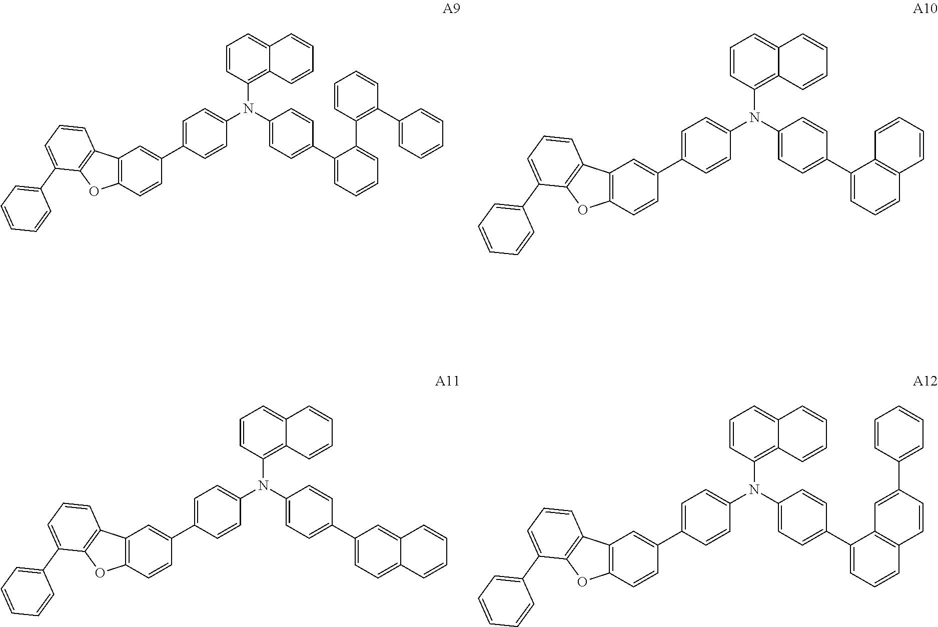 [SMILES: c1ccc(-c2ccccc2-c2ccccc2-c2ccc(N(c3ccc(-c4ccc5oc6c(-c7ccccc7)cccc6c5c4)cc3)c3cccc4ccccc34)cc2)cc1 ; c1ccc(-c2cccc3c2oc2ccc(-c4ccc(N(c5ccc(-c6cccc7ccccc67)cc5)c5cccc6ccccc56)cc4)cc23)cc1 ; c1ccc(-c2cccc3c2oc2ccc(-c4ccc(N(c5ccc(-c6ccc7ccccc7c6)cc5)c5cccc6ccccc56)cc4)cc23)cc1 ; c1ccc(-c2ccc3cccc(-c4ccc(N(c5ccc(-c6ccc7oc8c(-c9ccccc9)cccc8c7c6)cc5)c5cccc6ccccc56)cc4)c3c2)cc1]

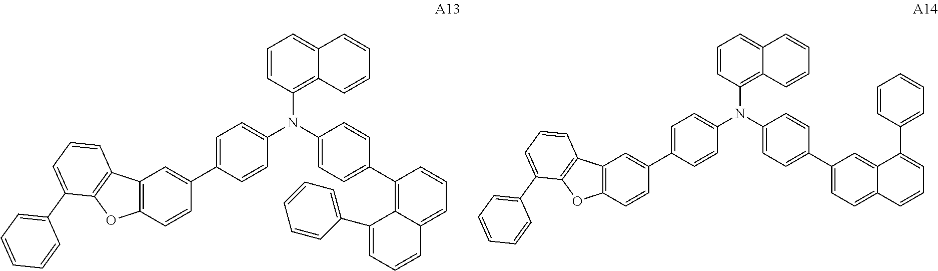

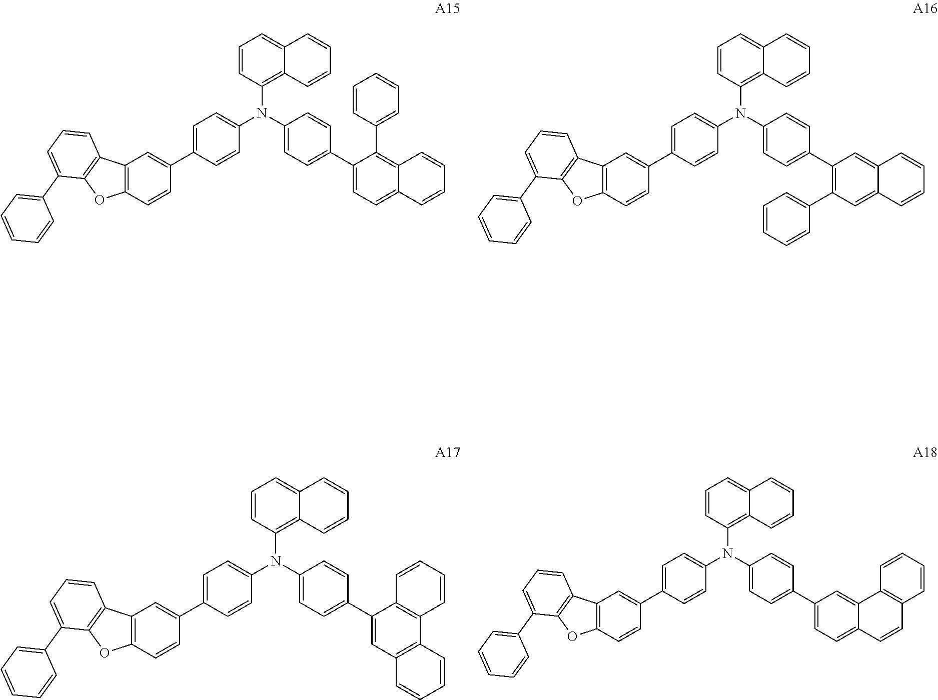

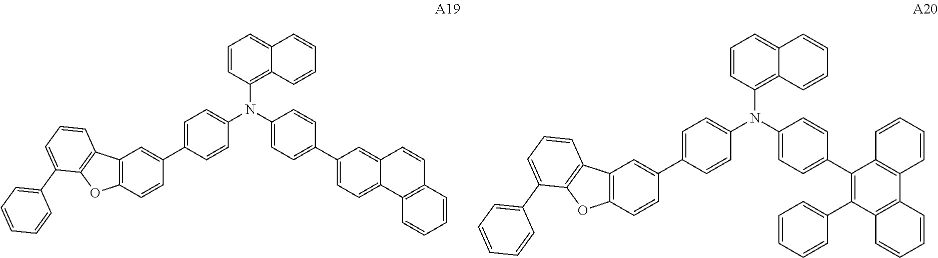

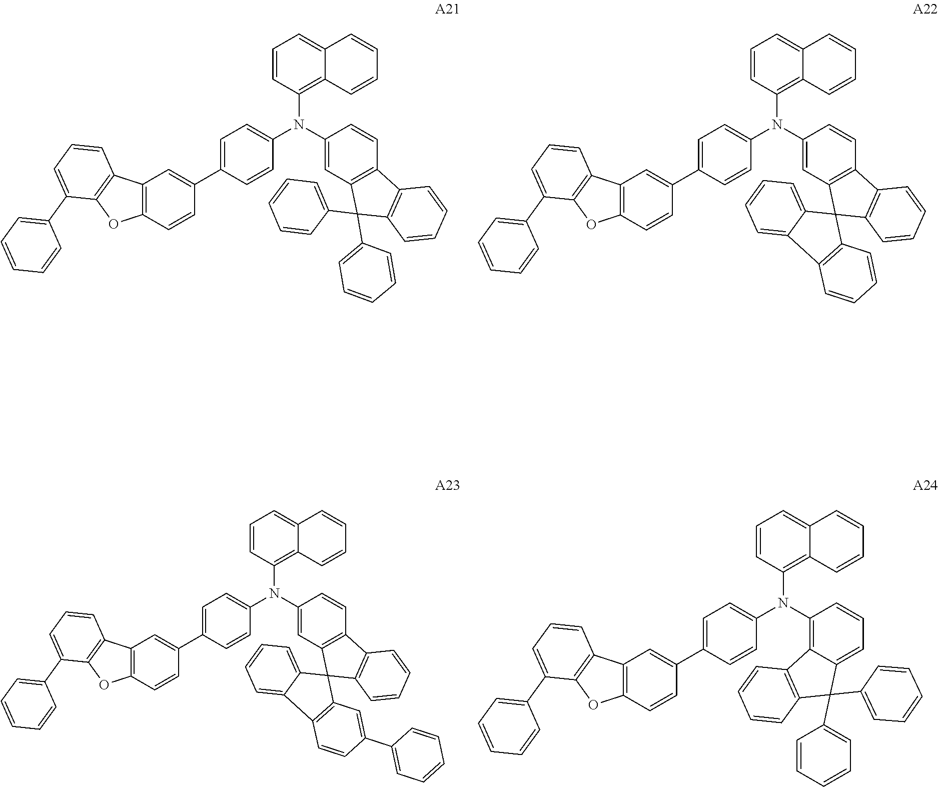 [SMILES: c1ccc(-c2cccc3c2oc2ccc(-c4ccc(N(c5ccc6c(c5)C(c5ccccc5)(c5ccccc5)c5ccccc5-6)c5cccc6ccccc56)cc4)cc23)cc1 ; c1ccc(-c2cccc3c2oc2ccc(-c4ccc(N(c5ccc6c(c5)C5(c7ccccc7-c7ccccc75)c5ccccc5-6)c5cccc6ccccc56)cc4)cc23)cc1 ; c1ccc(-c2ccc3c(c2)C2(c4ccccc4-3)c3ccccc3-c3ccc(N(c4ccc(-c5ccc6oc7c(-c8ccccc8)cccc7c6c5)cc4)c4cccc5ccccc45)cc32)cc1 ; c1ccc(-c2cccc3c2oc2ccc(-c4ccc(N(c5cccc6c5-c5ccccc5C6(c5ccccc5)c5ccccc5)c5cccc6ccccc56)cc4)cc23)cc1]

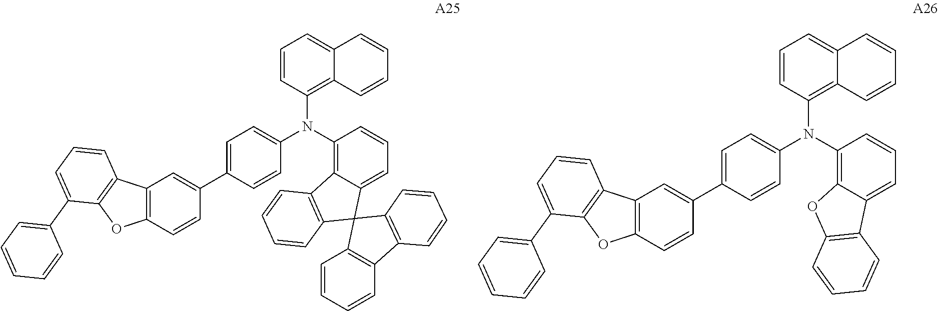 [SMILES: c1ccc(-c2cccc3c2oc2ccc(-c4ccc(N(c5cccc6c5-c5ccccc5C65c6ccccc6-c6ccccc65)c5cccc6ccccc56)cc4)cc23)cc1 ; c1ccc(-c2cccc3c2oc2ccc(-c4ccc(N(c5cccc6ccccc56)c5cccc6c5oc5ccccc56)cc4)cc23)cc1]

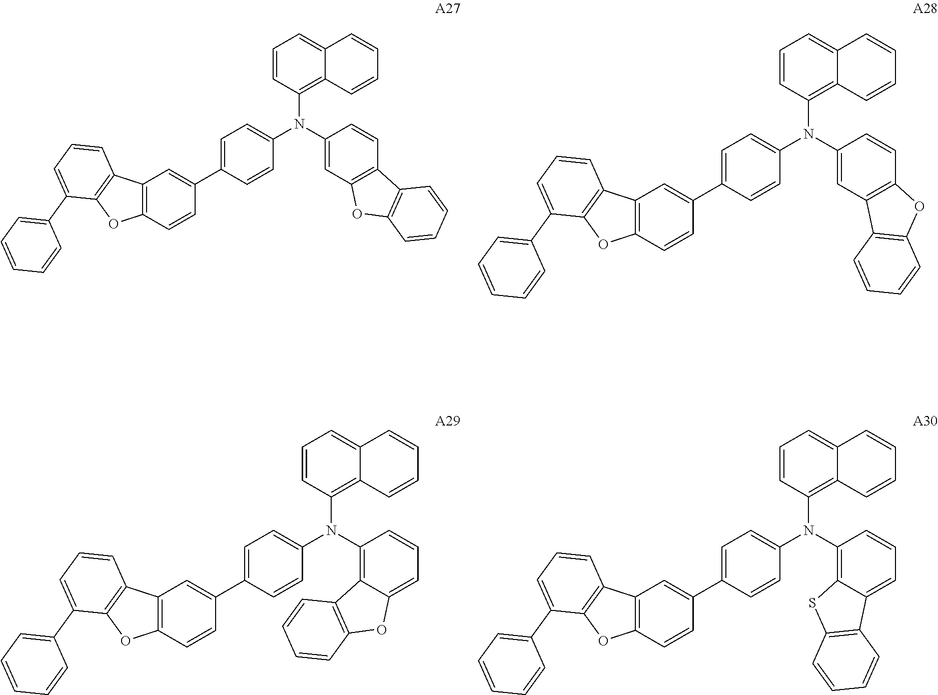 [SMILES: c1ccc(-c2cccc3c2oc2ccc(-c4ccc(N(c5ccc6c(c5)oc5ccccc56)c5cccc6ccccc56)cc4)cc23)cc1 ; c1ccc(-c2cccc3c2oc2ccc(-c4ccc(N(c5ccc6oc7ccccc7c6c5)c5cccc6ccccc56)cc4)cc23)cc1 ; c1ccc(-c2cccc3c2oc2ccc(-c4ccc(N(c5cccc6ccccc56)c5cccc6oc7ccccc7c56)cc4)cc23)cc1 ; c1ccc(-c2cccc3c2oc2ccc(-c4ccc(N(c5cccc6ccccc56)c5cccc6c5sc5ccccc56)cc4)cc23)cc1]

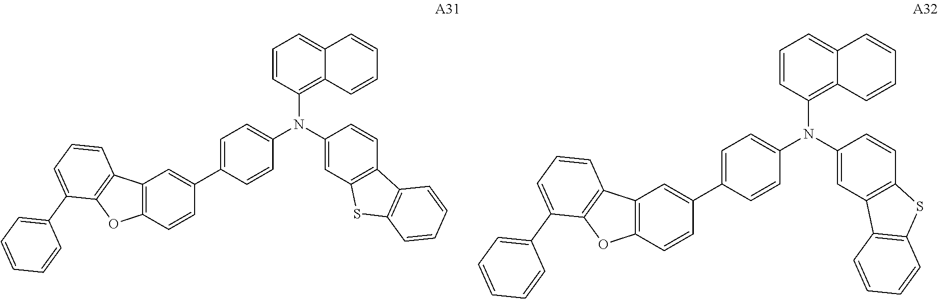 [SMILES: c1ccc(-c2cccc3c2oc2ccc(-c4ccc(N(c5ccc6c(c5)sc5ccccc56)c5cccc6ccccc56)cc4)cc23)cc1 ; c1ccc(-c2cccc3c2oc2ccc(-c4ccc(N(c5ccc6sc7ccccc7c6c5)c5cccc6ccccc56)cc4)cc23)cc1]

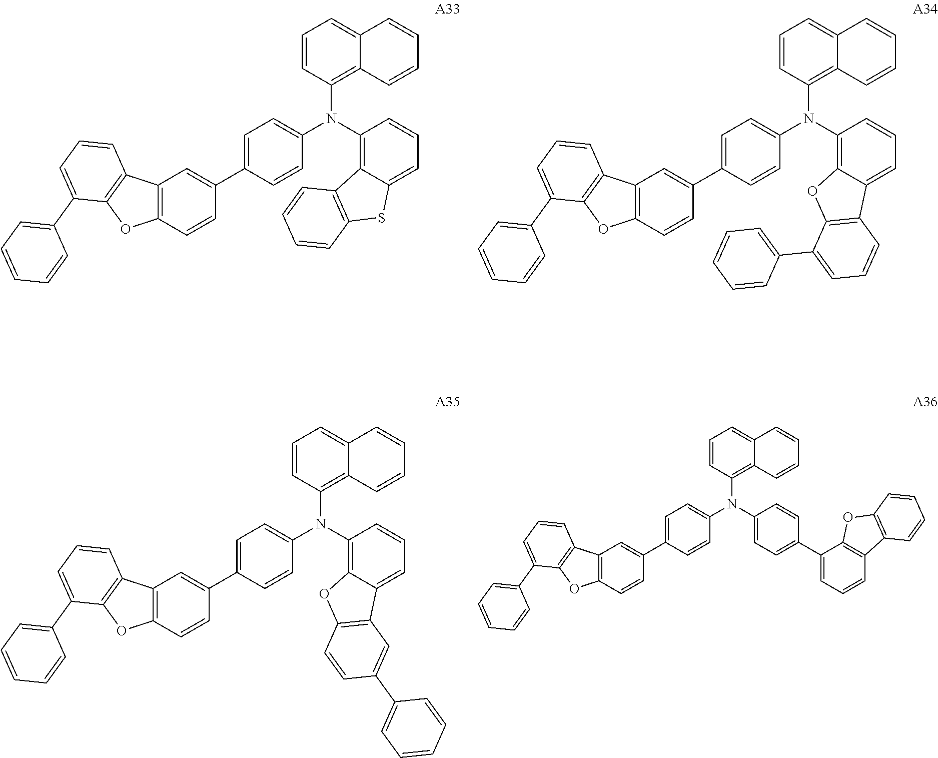 [SMILES: c1ccc(-c2cccc3c2oc2ccc(-c4ccc(N(c5cccc6ccccc56)c5cccc6sc7ccccc7c56)cc4)cc23)cc1 ; c1ccc(-c2cccc3c2oc2ccc(-c4ccc(N(c5cccc6ccccc56)c5cccc6c5oc5c(-c7ccccc7)cccc56)cc4)cc23)cc1 ; c1ccc(-c2ccc3oc4c(N(c5ccc(-c6ccc7oc8c(-c9ccccc9)cccc8c7c6)cc5)c5cccc6ccccc56)cccc4c3c2)cc1 ; c1ccc(-c2cccc3c2oc2ccc(-c4ccc(N(c5ccc(-c6cccc7c6oc6ccccc67)cc5)c5cccc6ccccc56)cc4)cc23)cc1]

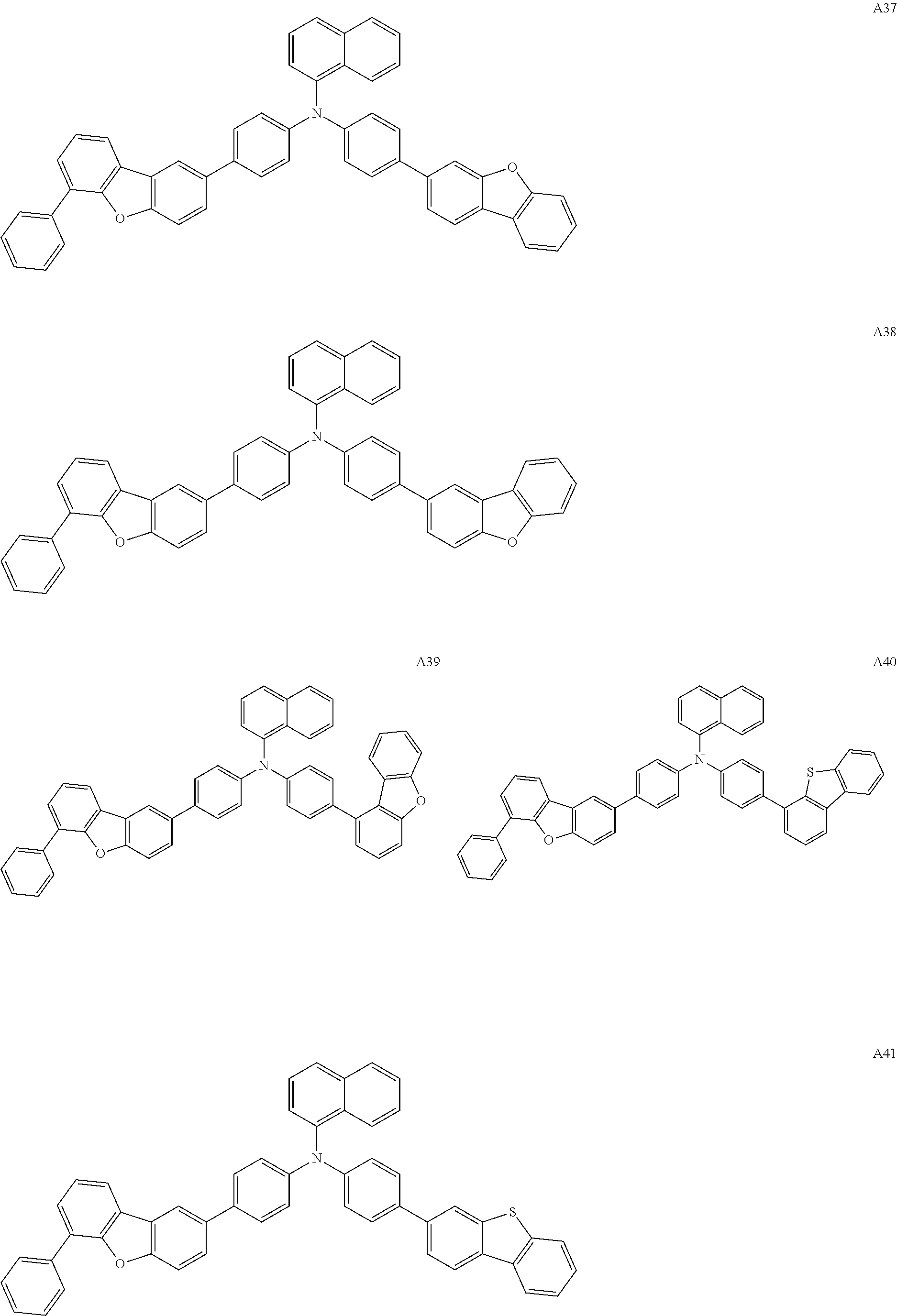 [SMILES: c1ccc(-c2cccc3c2oc2ccc(-c4ccc(N(c5ccc(-c6ccc7c(c6)oc6ccccc67)cc5)c5cccc6ccccc56)cc4)cc23)cc1 ; c1ccc(-c2cccc3c2oc2ccc(-c4ccc(N(c5ccc(-c6ccc7oc8ccccc8c7c6)cc5)c5cccc6ccccc56)cc4)cc23)cc1 ; c1ccc(-c2cccc3c2oc2ccc(-c4ccc(N(c5ccc(-c6cccc7oc8ccccc8c67)cc5)c5cccc6ccccc56)cc4)cc23)cc1 ; c1ccc(-c2cccc3c2oc2ccc(-c4ccc(N(c5ccc(-c6cccc7c6sc6ccccc67)cc5)c5cccc6ccccc56)cc4)cc23)cc1 ; c1ccc(-c2cccc3c2oc2ccc(-c4ccc(N(c5ccc(-c6ccc7c(c6)sc6ccccc67)cc5)c5cccc6ccccc56)cc4)cc23)cc1]

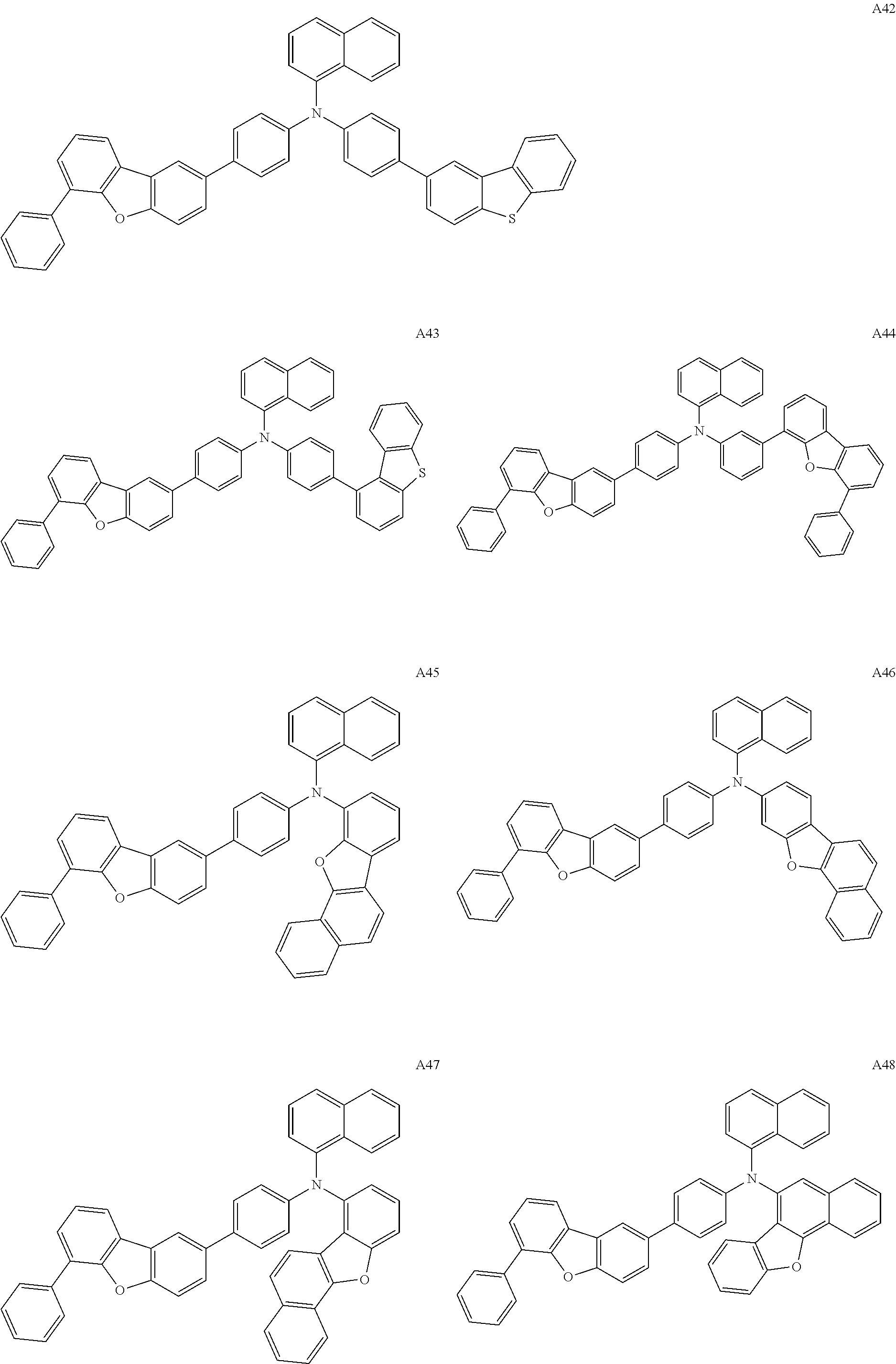

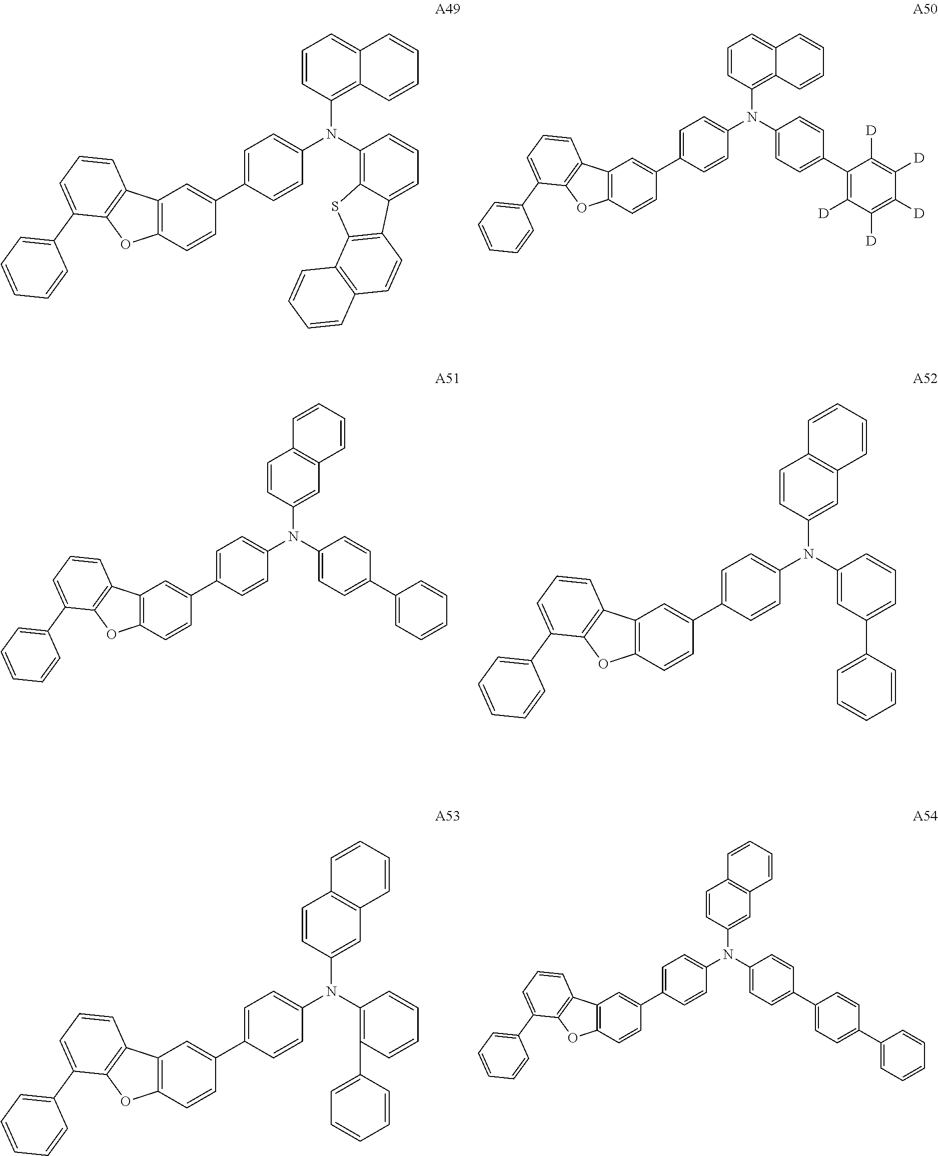

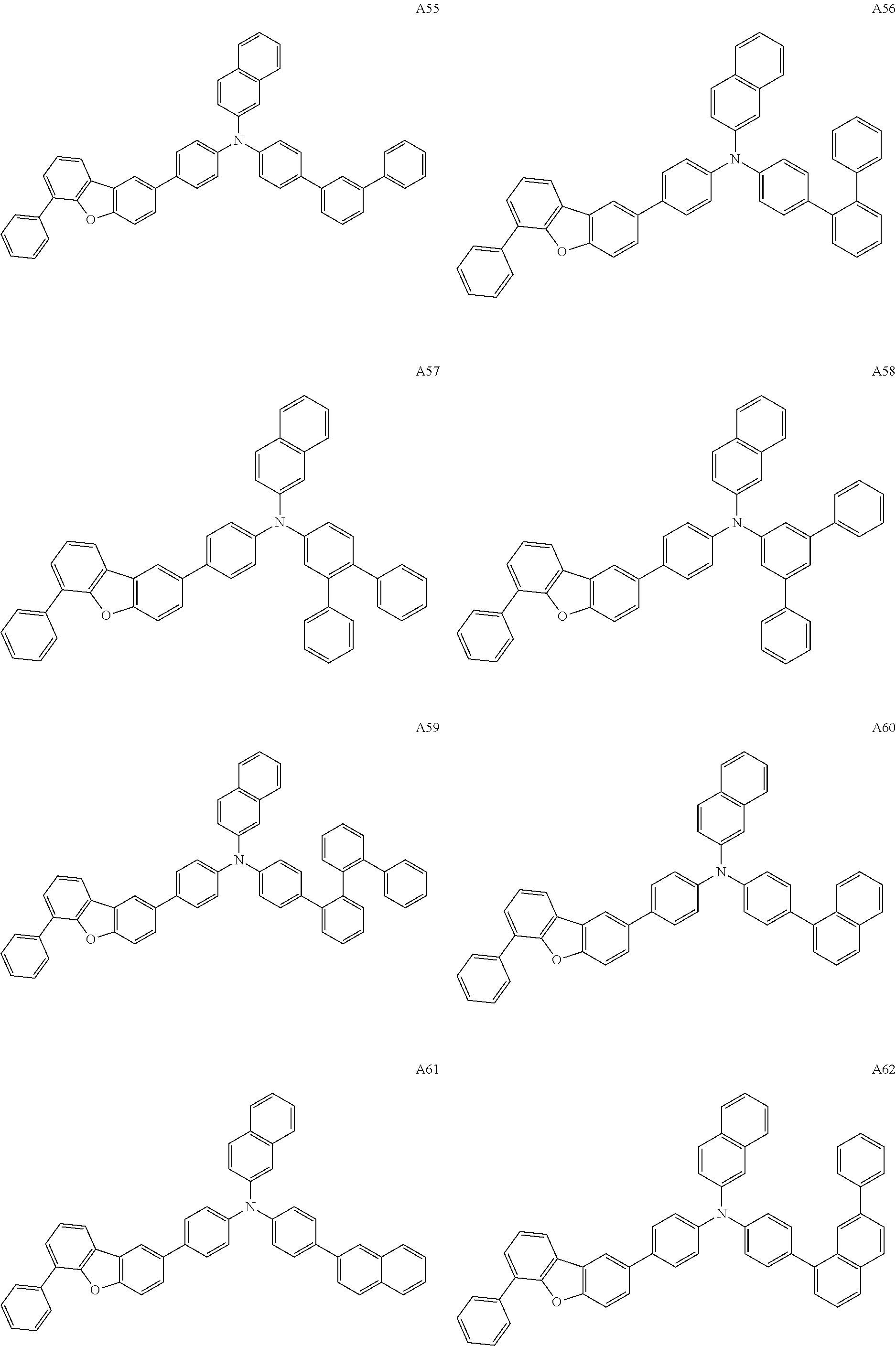

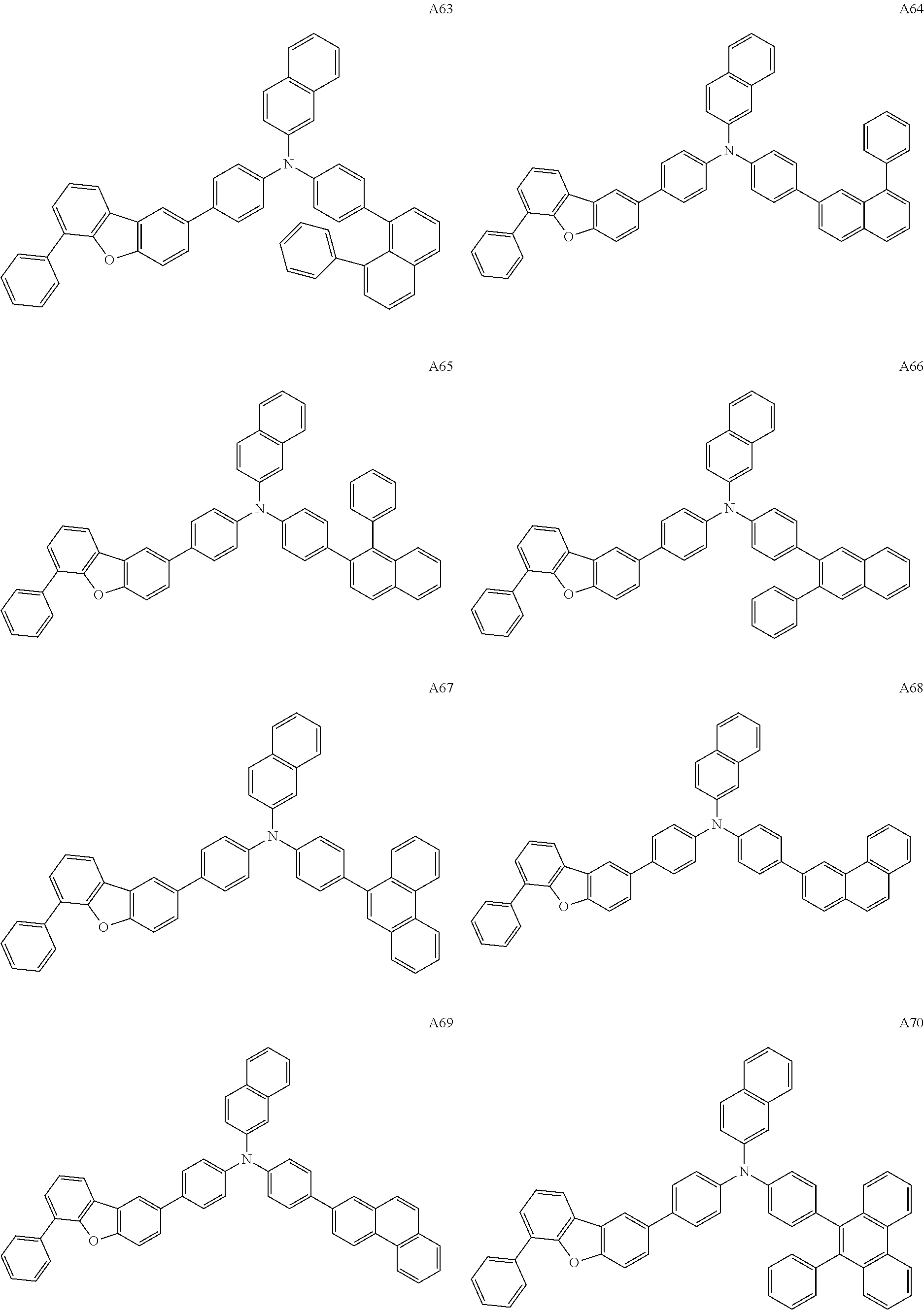

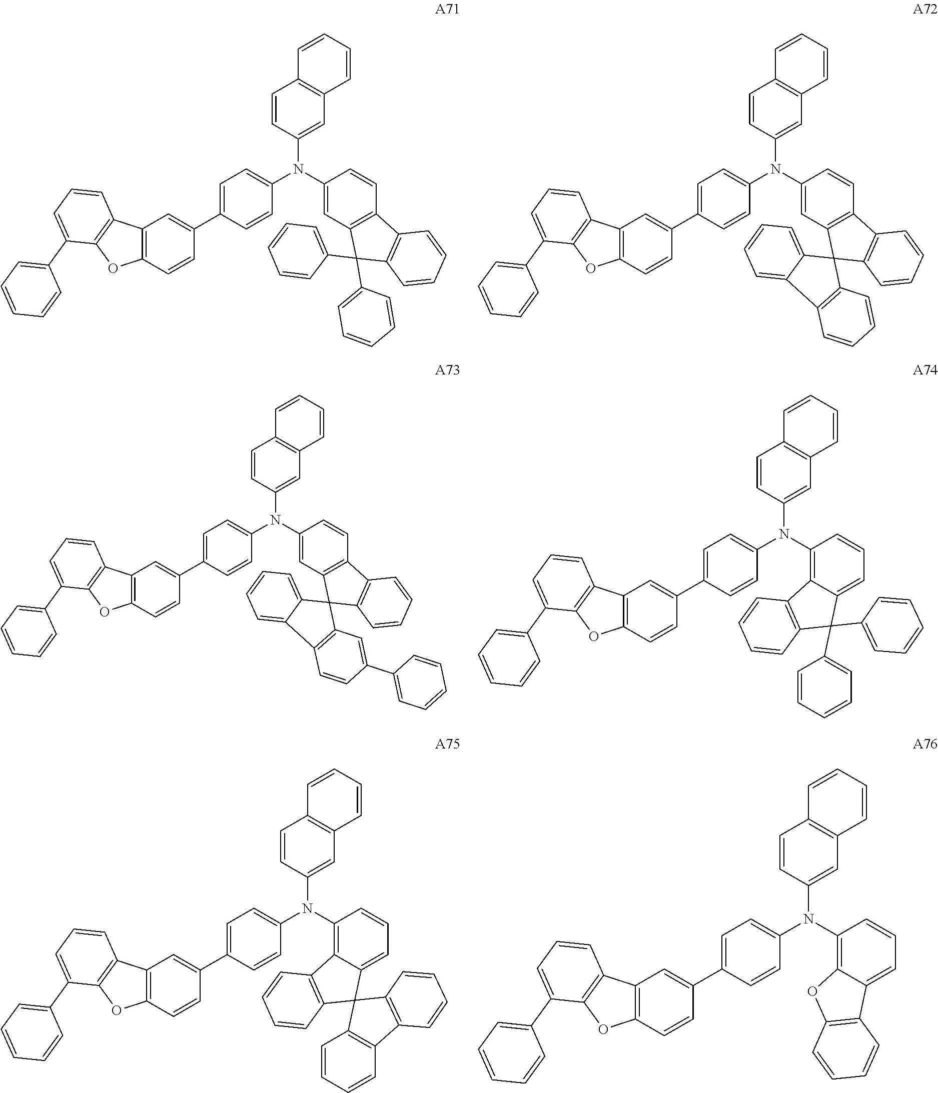

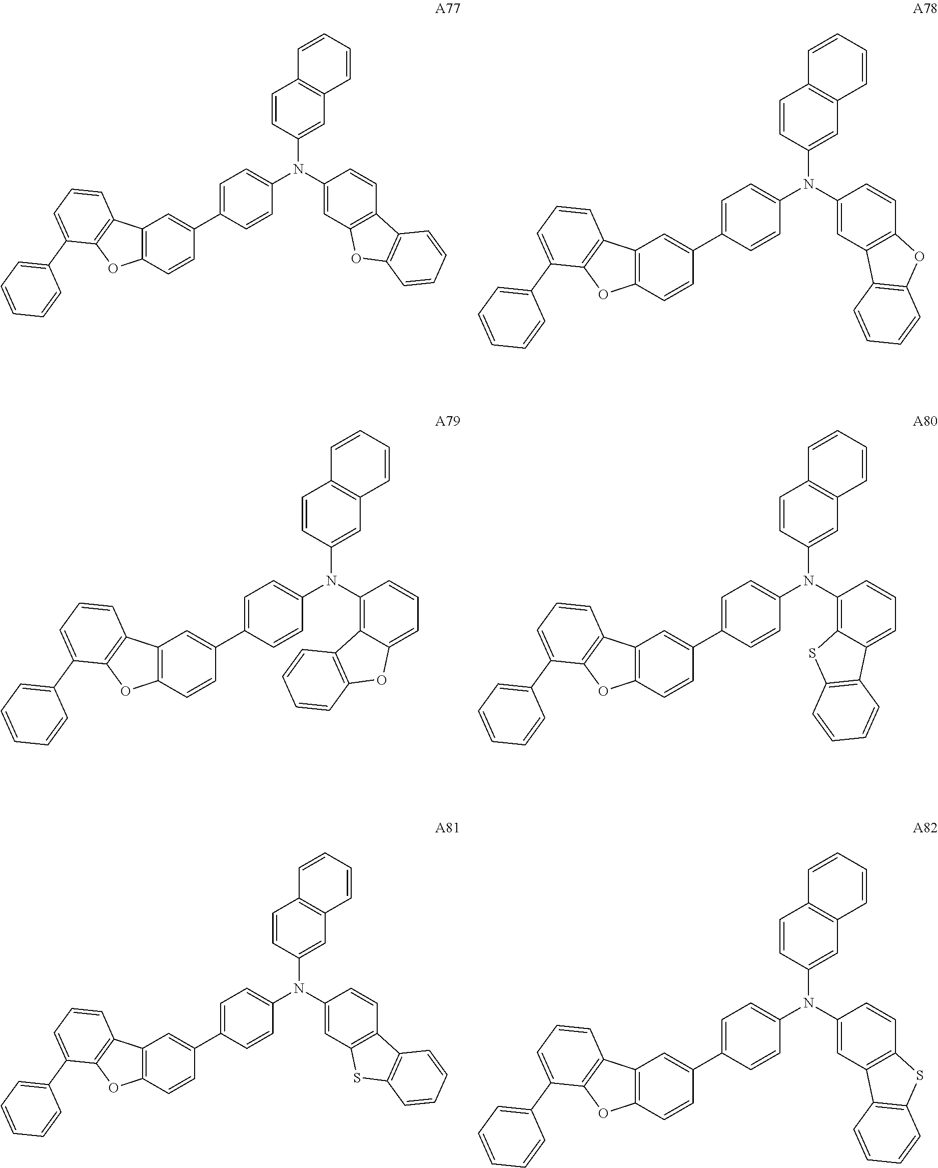 [SMILES: c1ccc(-c2cccc3c2oc2ccc(-c4ccc(N(c5ccc6ccccc6c5)c5ccc6c(c5)oc5ccccc56)cc4)cc23)cc1 ; c1ccc(-c2cccc3c2oc2ccc(-c4ccc(N(c5ccc6ccccc6c5)c5ccc6oc7ccccc7c6c5)cc4)cc23)cc1 ; c1ccc(-c2cccc3c2oc2ccc(-c4ccc(N(c5ccc6ccccc6c5)c5cccc6oc7ccccc7c56)cc4)cc23)cc1 ; c1ccc(-c2cccc3c2oc2ccc(-c4ccc(N(c5ccc6ccccc6c5)c5cccc6c5sc5ccccc56)cc4)cc23)cc1 ; c1ccc(-c2cccc3c2oc2ccc(-c4ccc(N(c5ccc6ccccc6c5)c5ccc6c(c5)sc5ccccc56)cc4)cc23)cc1 ; c1ccc(-c2cccc3c2oc2ccc(-c4ccc(N(c5ccc6ccccc6c5)c5ccc6sc7ccccc7c6c5)cc4)cc23)cc1]

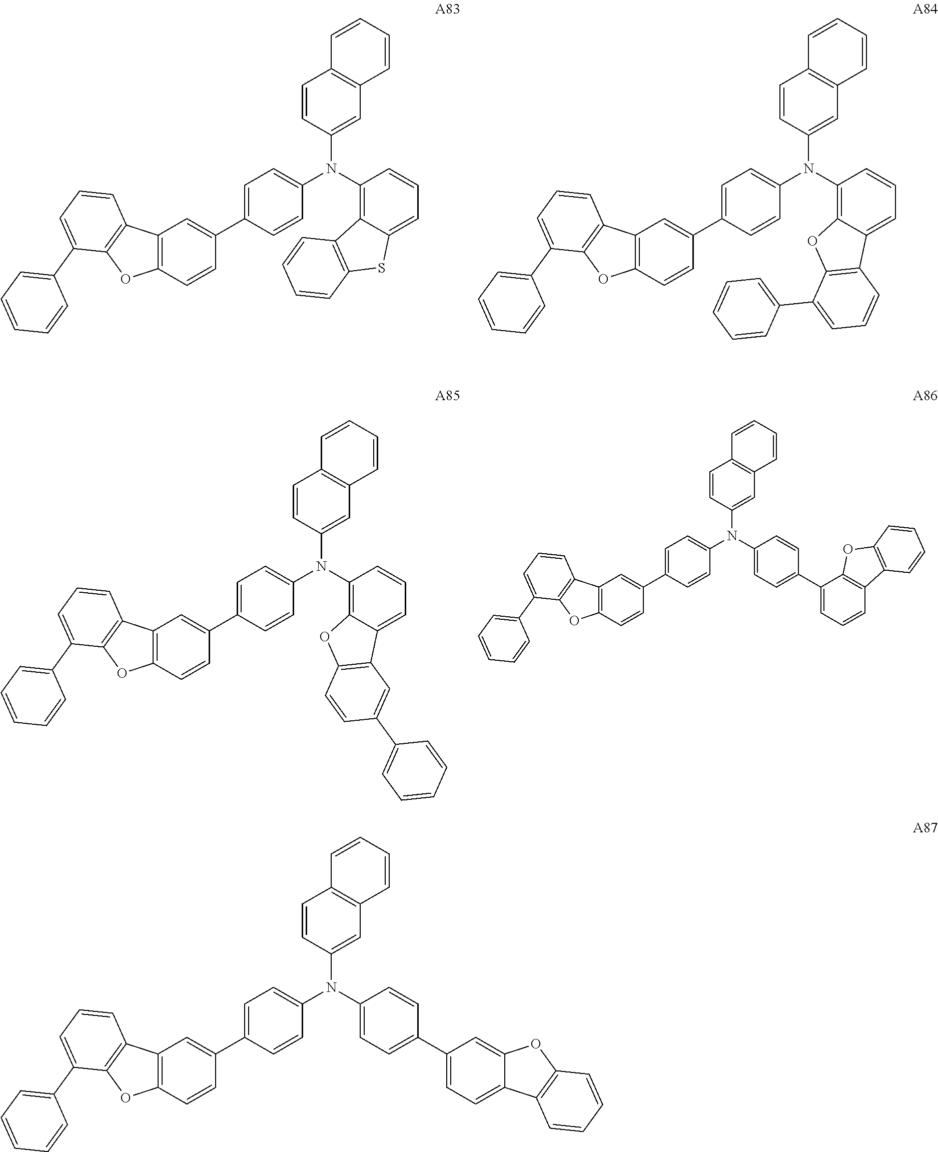 [SMILES: c1ccc(-c2cccc3c2oc2ccc(-c4ccc(N(c5ccc6ccccc6c5)c5cccc6sc7ccccc7c56)cc4)cc23)cc1 ; c1ccc(-c2cccc3c2oc2ccc(-c4ccc(N(c5ccc6ccccc6c5)c5cccc6c5oc5c(-c7ccccc7)cccc56)cc4)cc23)cc1 ; c1ccc(-c2ccc3oc4c(N(c5ccc(-c6ccc7oc8c(-c9ccccc9)cccc8c7c6)cc5)c5ccc6ccccc6c5)cccc4c3c2)cc1 ; c1ccc(-c2cccc3c2oc2ccc(-c4ccc(N(c5ccc(-c6cccc7c6oc6ccccc67)cc5)c5ccc6ccccc6c5)cc4)cc23)cc1 ; c1ccc(-c2cccc3c2oc2ccc(-c4ccc(N(c5ccc(-c6ccc7c(c6)oc6ccccc67)cc5)c5ccc6ccccc6c5)cc4)cc23)cc1]

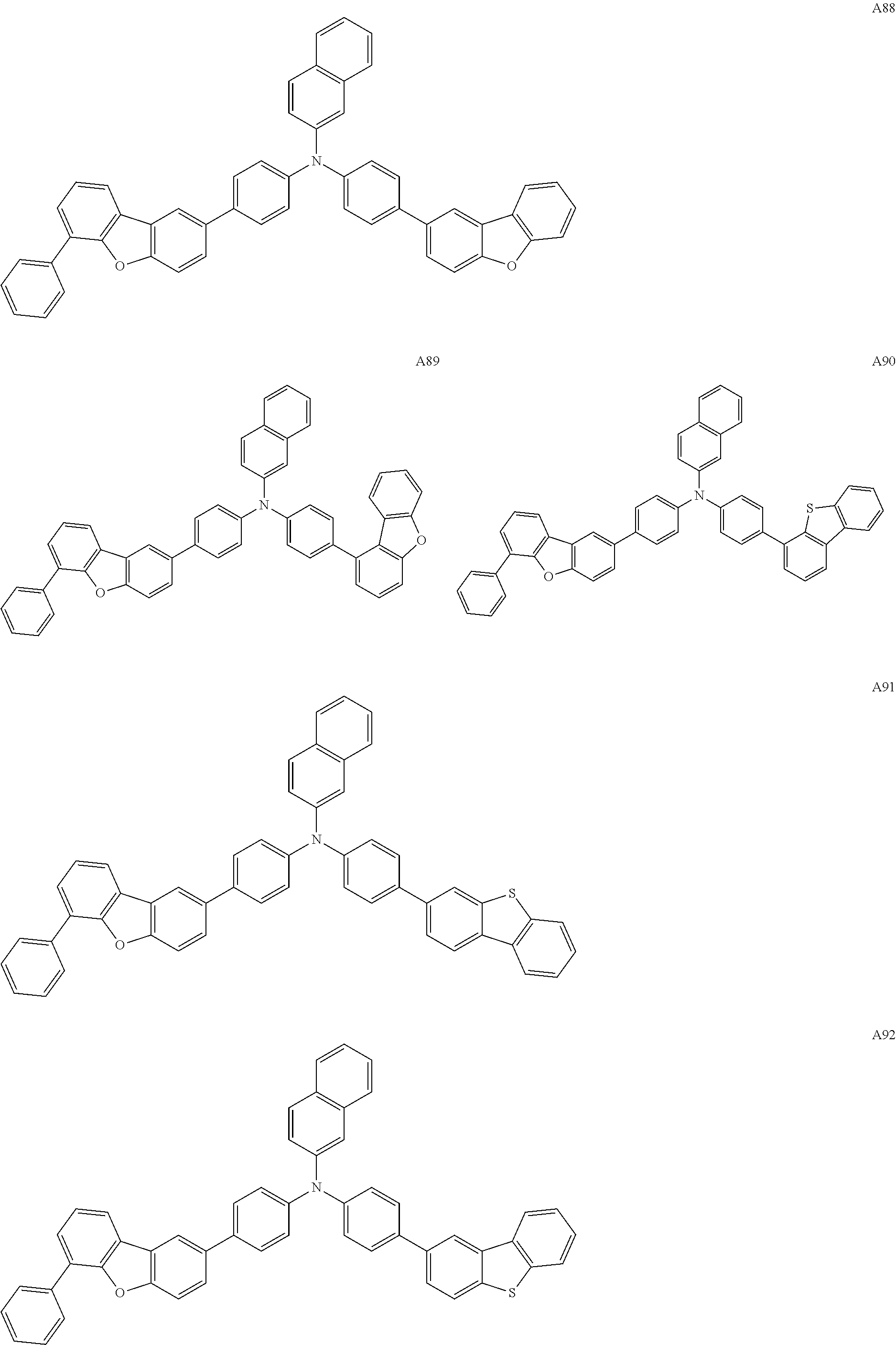 [SMILES: c1ccc(-c2cccc3c2oc2ccc(-c4ccc(N(c5ccc(-c6ccc7oc8ccccc8c7c6)cc5)c5ccc6ccccc6c5)cc4)cc23)cc1 ; c1ccc(-c2cccc3c2oc2ccc(-c4ccc(N(c5ccc(-c6cccc7oc8ccccc8c67)cc5)c5ccc6ccccc6c5)cc4)cc23)cc1 ; c1ccc(-c2cccc3c2oc2ccc(-c4ccc(N(c5ccc(-c6cccc7c6sc6ccccc67)cc5)c5ccc6ccccc6c5)cc4)cc23)cc1 ; c1ccc(-c2cccc3c2oc2ccc(-c4ccc(N(c5ccc(-c6ccc7c(c6)sc6ccccc67)cc5)c5ccc6ccccc6c5)cc4)cc23)cc1 ; c1ccc(-c2cccc3c2oc2ccc(-c4ccc(N(c5ccc(-c6ccc7sc8ccccc8c7c6)cc5)c5ccc6ccccc6c5)cc4)cc23)cc1]

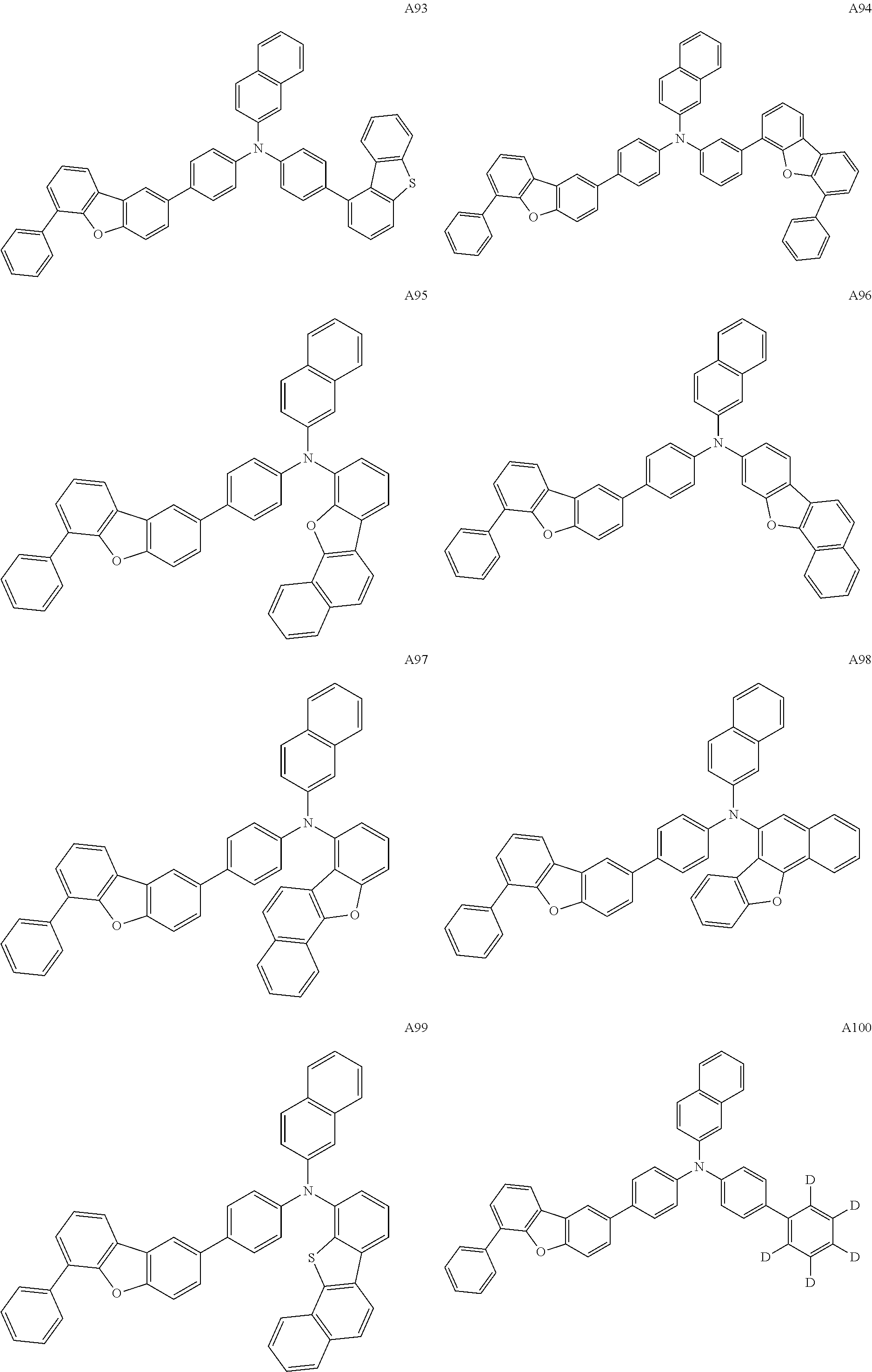

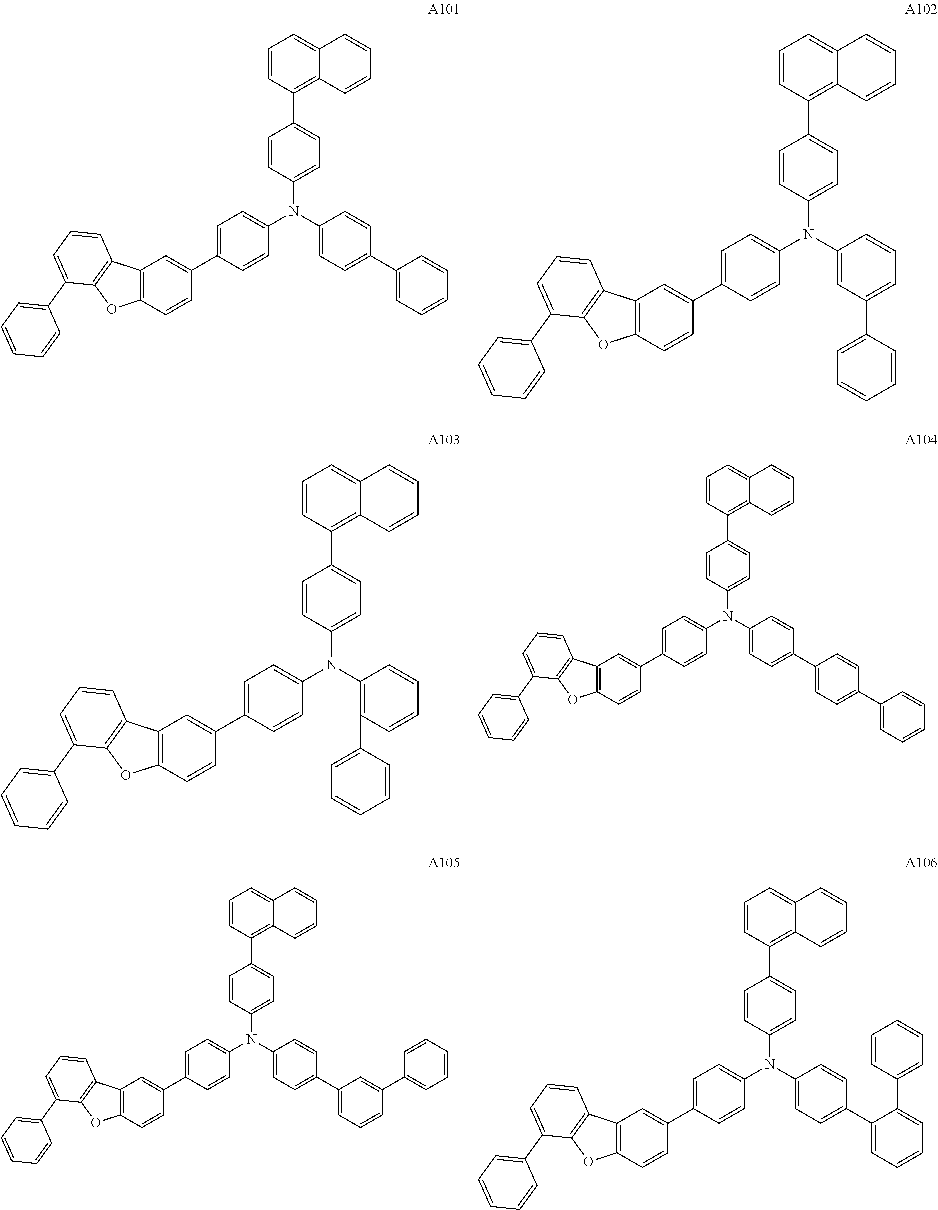

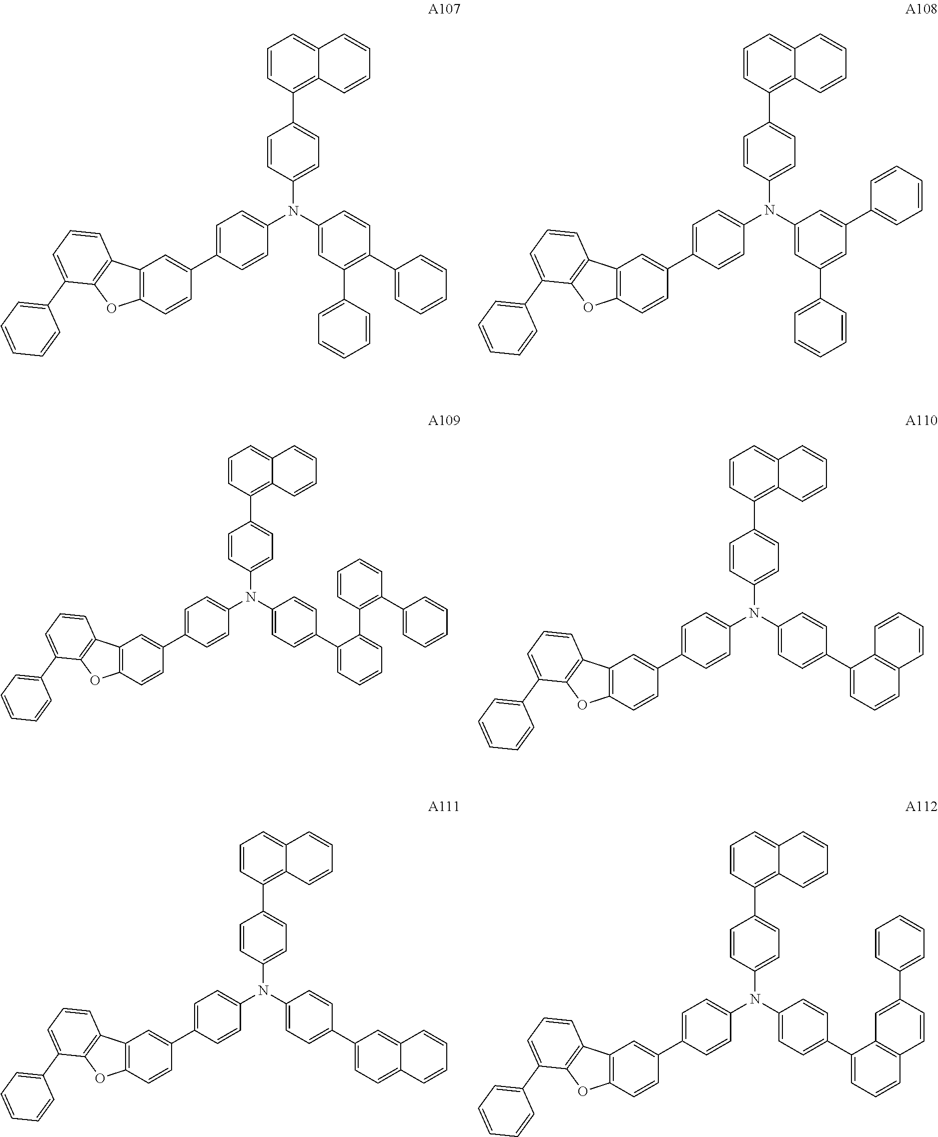

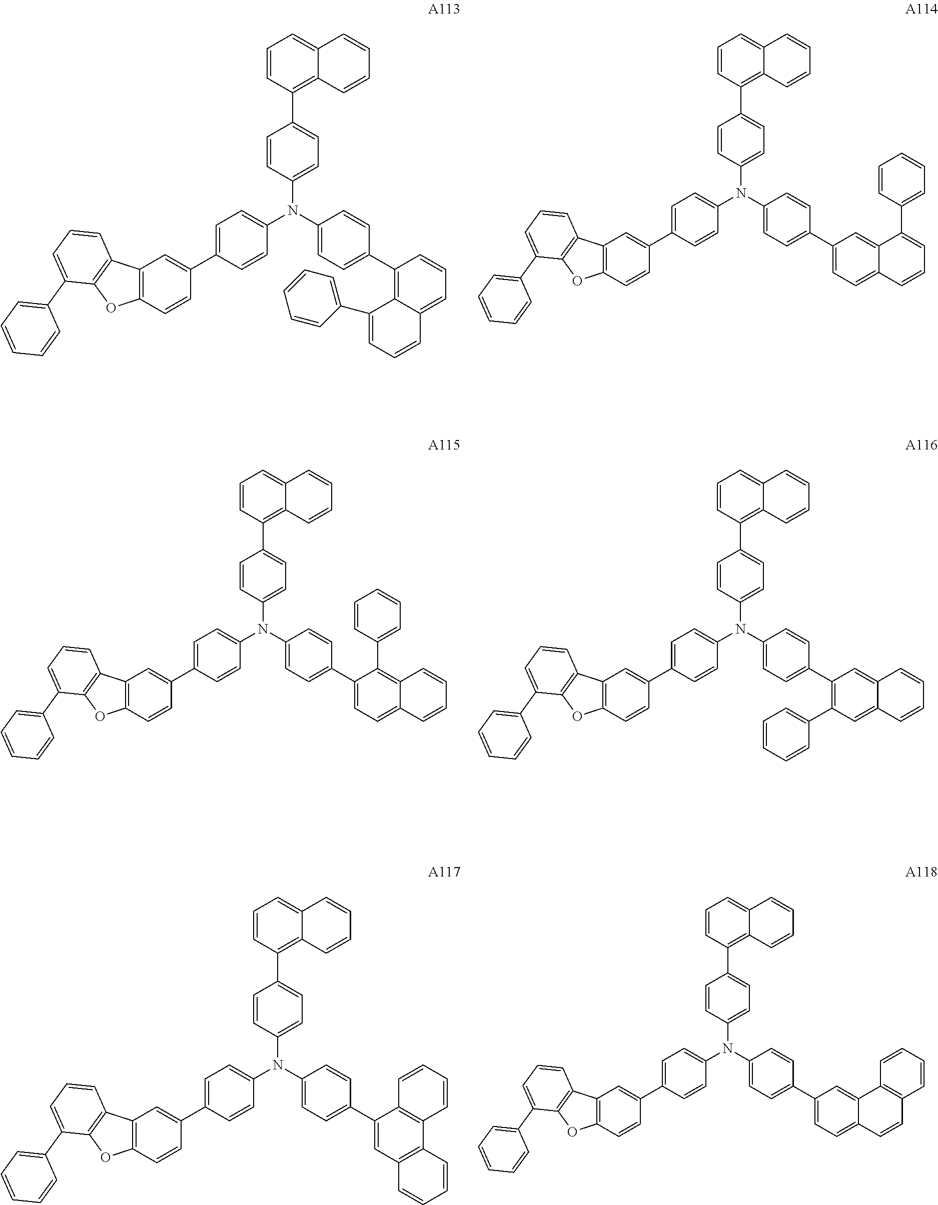

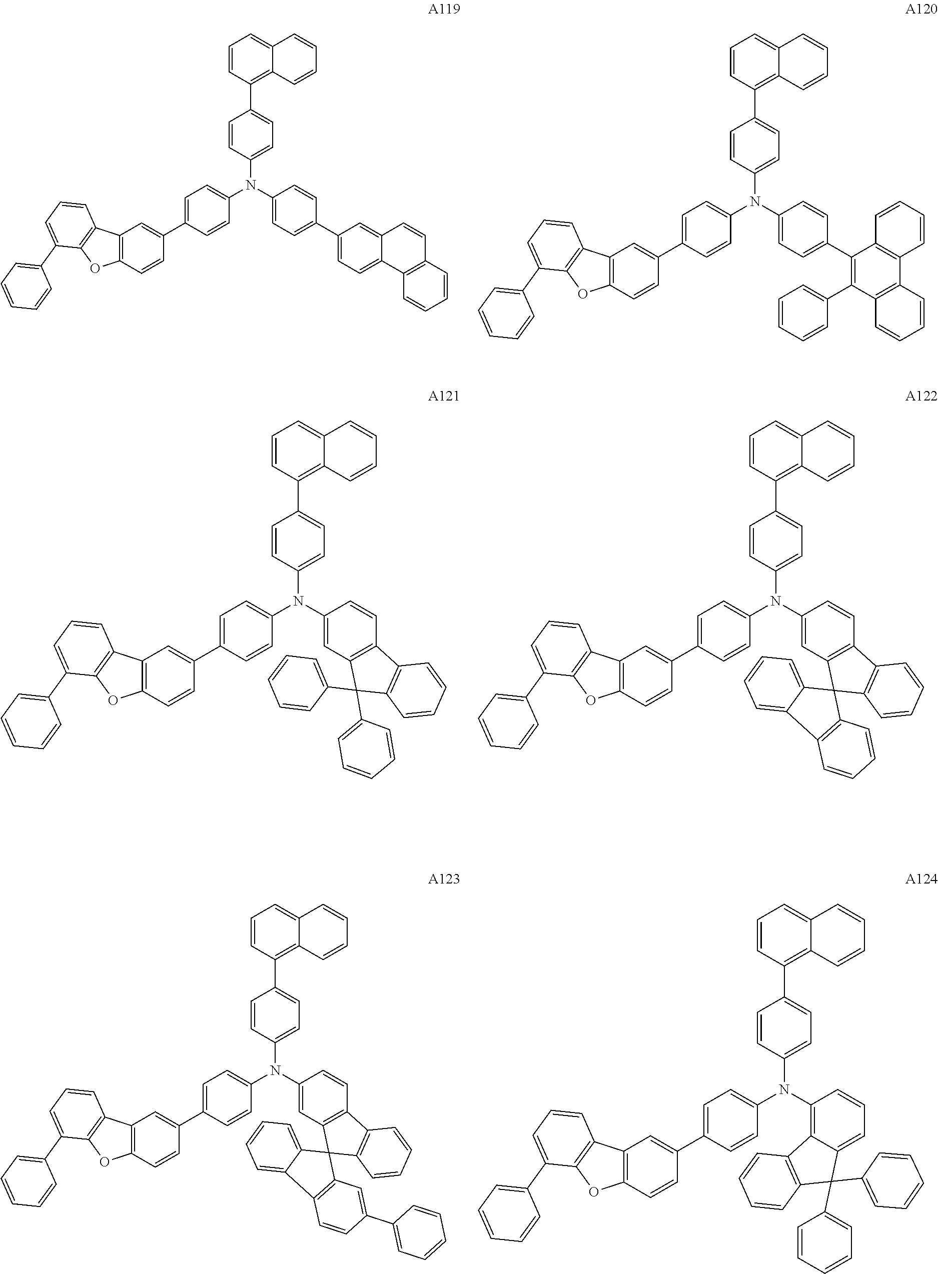

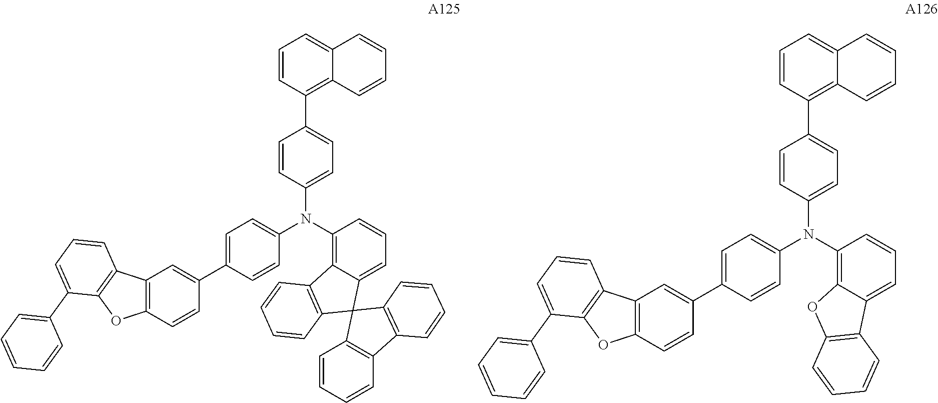 [SMILES: c1ccc(-c2cccc3c2oc2ccc(-c4ccc(N(c5ccc(-c6cccc7ccccc67)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6cccc7ccccc67)cc5)c5cccc6c5oc5ccccc56)cc4)cc23)cc1]

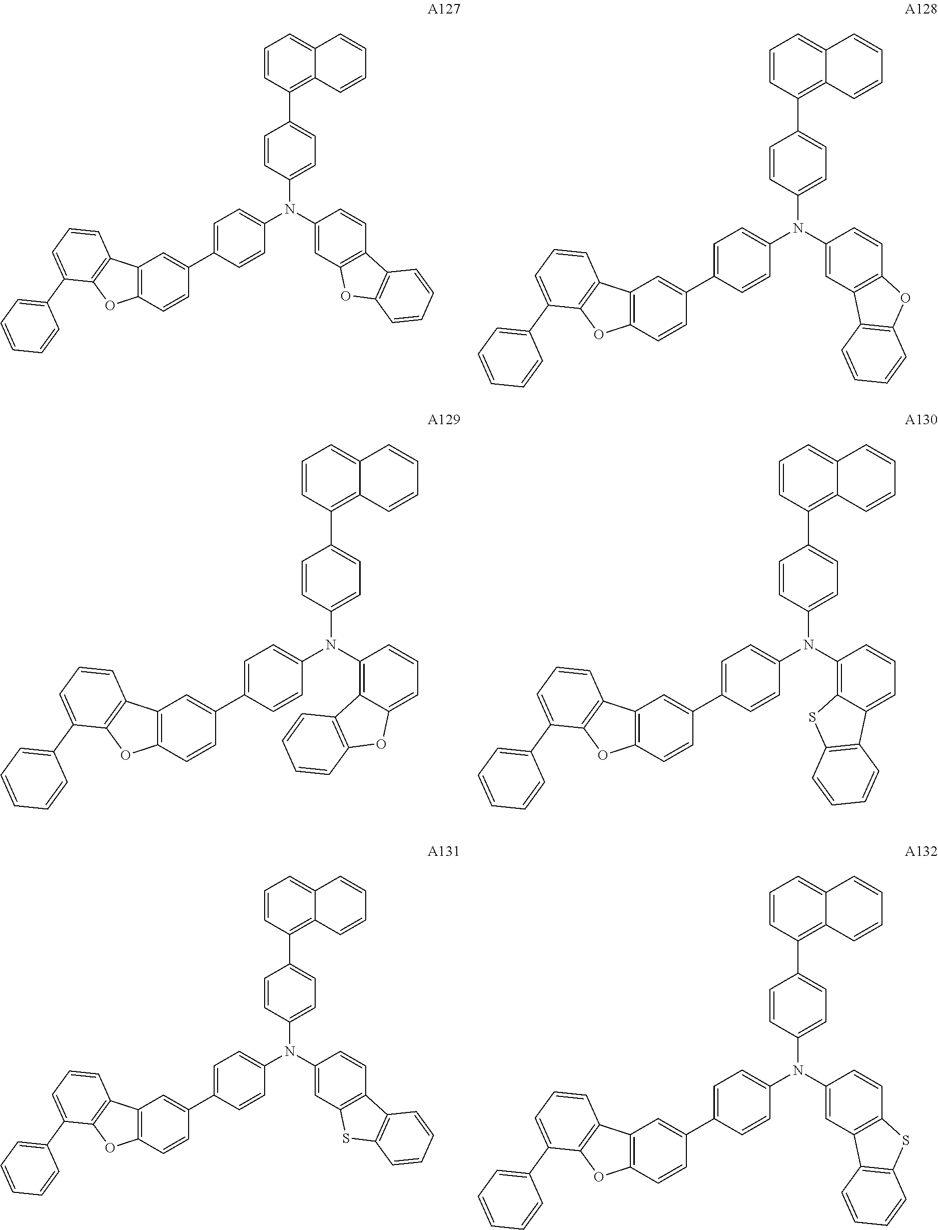

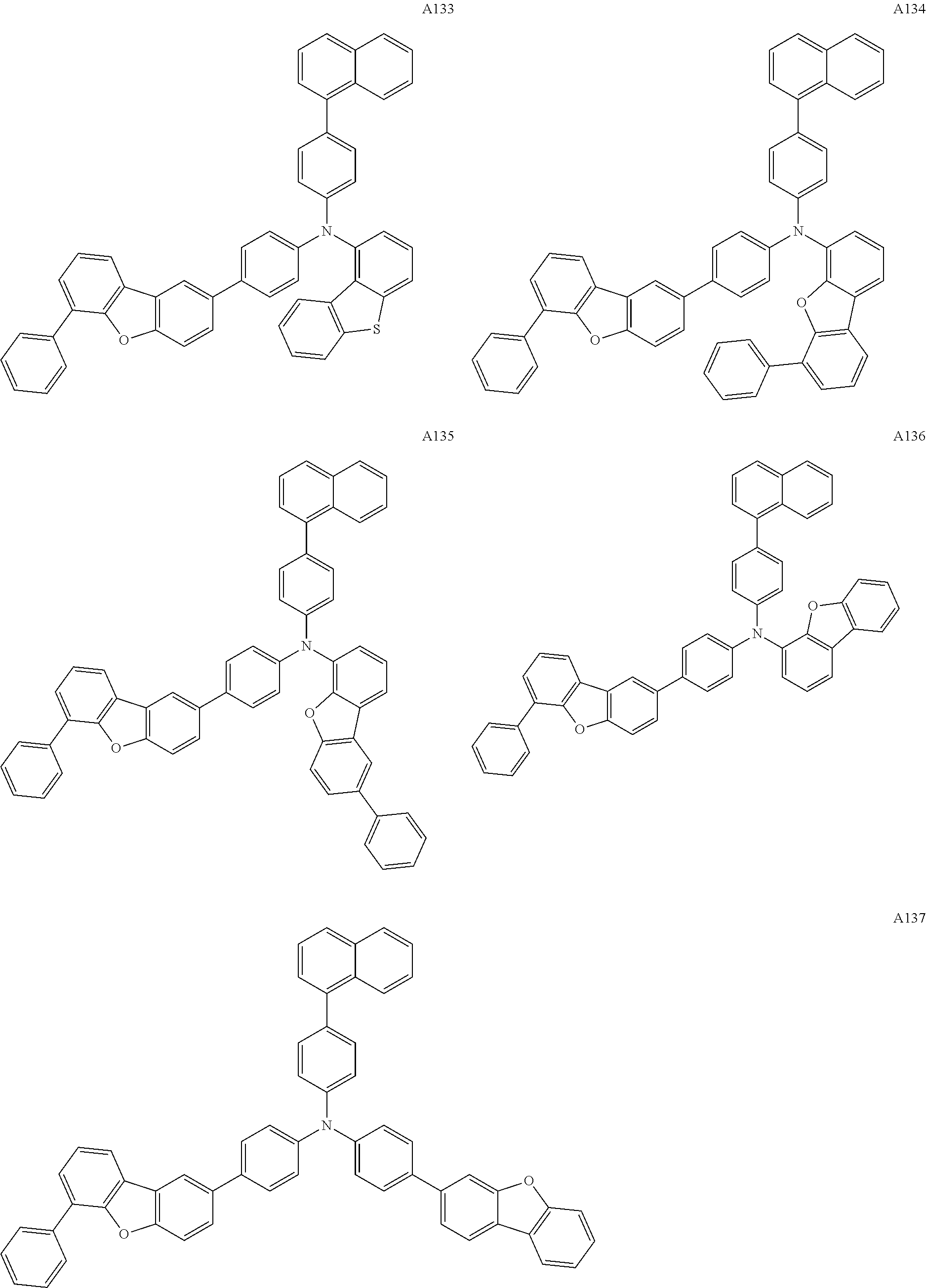 [SMILES: c1ccc(-c2cccc3c2oc2ccc(-c4ccc(N(c5ccc(-c6cccc7ccccc67)cc5)c5cccc6sc7ccccc7c56)cc4)cc23)cc1 ; c1ccc(-c2cccc3c2oc2ccc(-c4ccc(N(c5ccc(-c6cccc7ccccc67)cc5)c5cccc6c5oc5c(-c7ccccc7)cccc56)cc4)cc23)cc1 ; c1ccc(-c2ccc3oc4c(N(c5ccc(-c6ccc7oc8c(-c9ccccc9)cccc8c7c6)cc5)c5ccc(-c6cccc7ccccc67)cc5)cccc4c3c2)cc1 ; c1ccc(-c2cccc3c2oc2ccc(-c4ccc(N(c5ccc(-c6cccc7ccccc67)cc5)c5cccc6c5oc5ccccc56)cc4)cc23)cc1 ; c1ccc(-c2cccc3c2oc2ccc(-c4ccc(N(c5ccc(-c6ccc7c(c6)oc6ccccc67)cc5)c5ccc(-c6cccc7ccccc67)cc5)cc4)cc23)cc1]

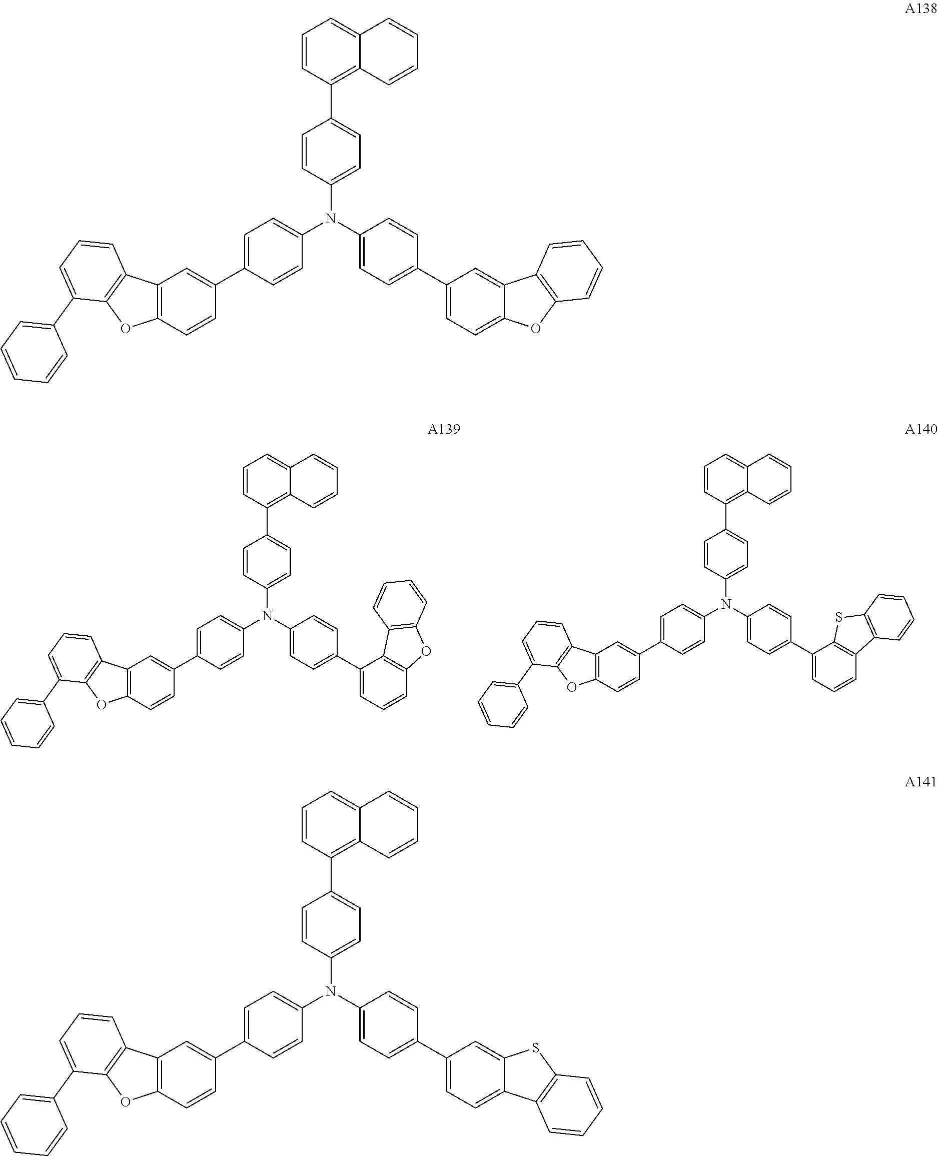 [SMILES: c1ccc(-c2cccc3c2oc2ccc(-c4ccc(N(c5ccc(-c6ccc7oc8ccccc8c7c6)cc5)c5ccc(-c6cccc7ccccc67)cc5)cc4)cc23)cc1 ; c1ccc(-c2cccc3c2oc2ccc(-c4ccc(N(c5ccc(-c6cccc7ccccc67)cc5)c5ccc(-c6cccc7oc8ccccc8c67)cc5)cc4)cc23)cc1 ; c1ccc(-c2cccc3c2oc2ccc(-c4ccc(N(c5ccc(-c6cccc7ccccc67)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6cccc7ccccc67)cc5)cc4)cc23)cc1]

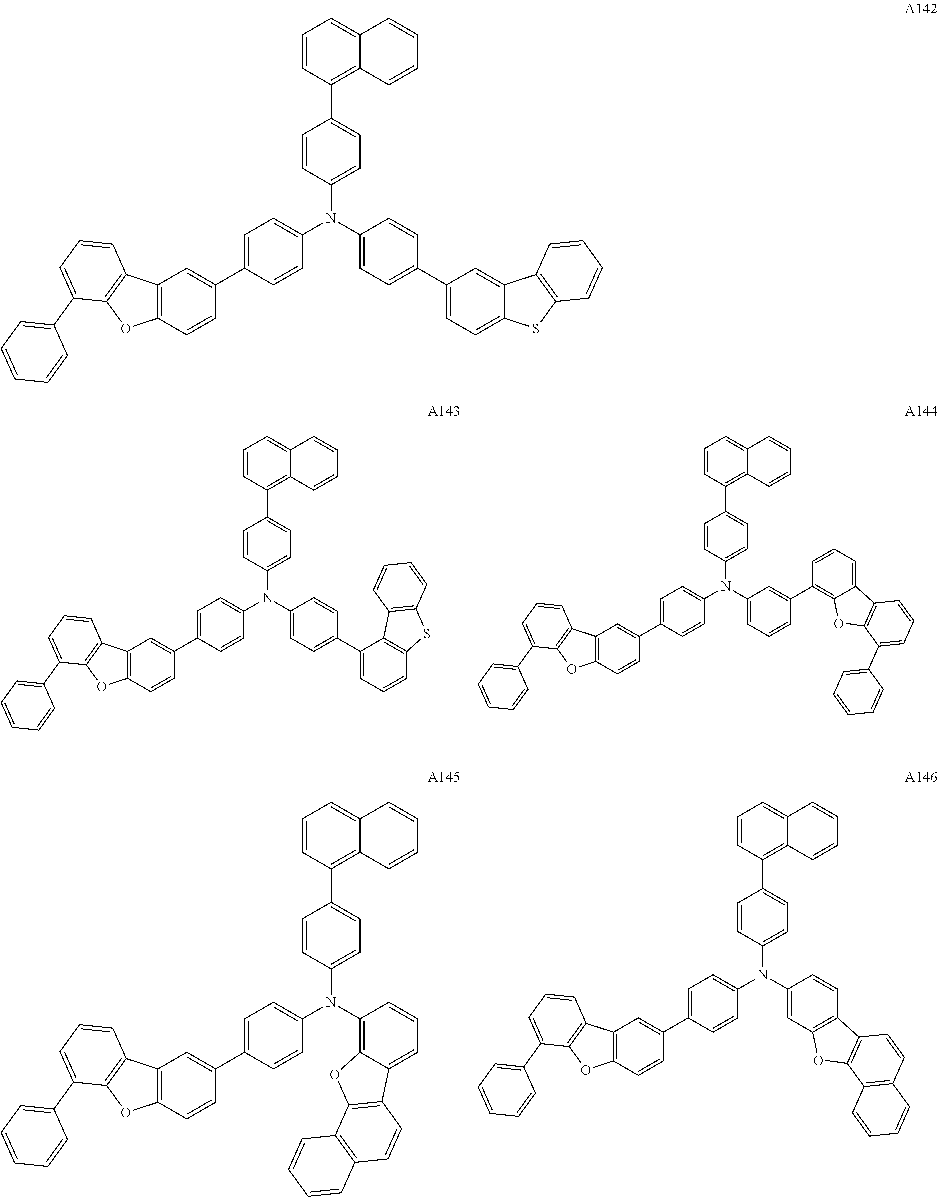

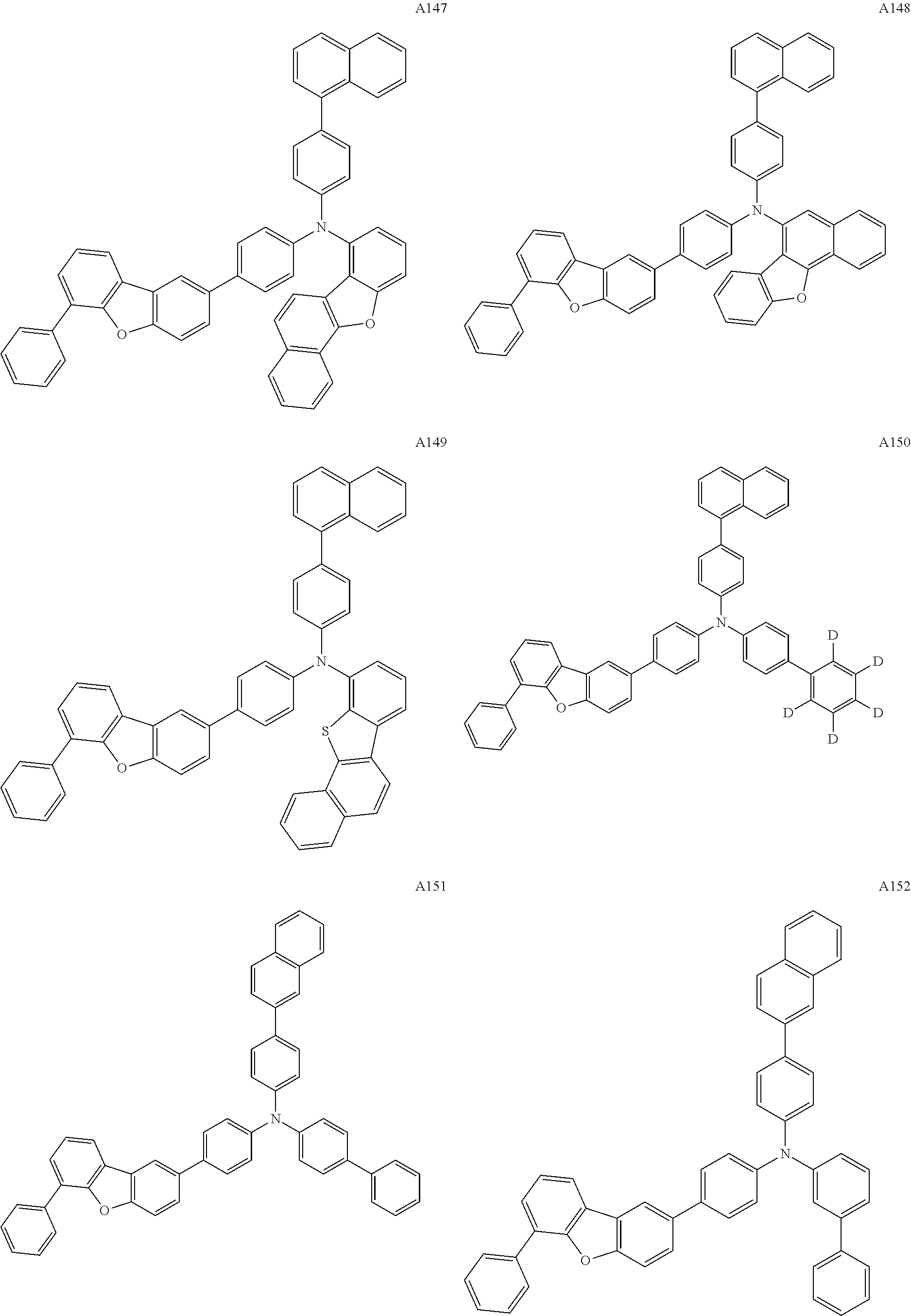

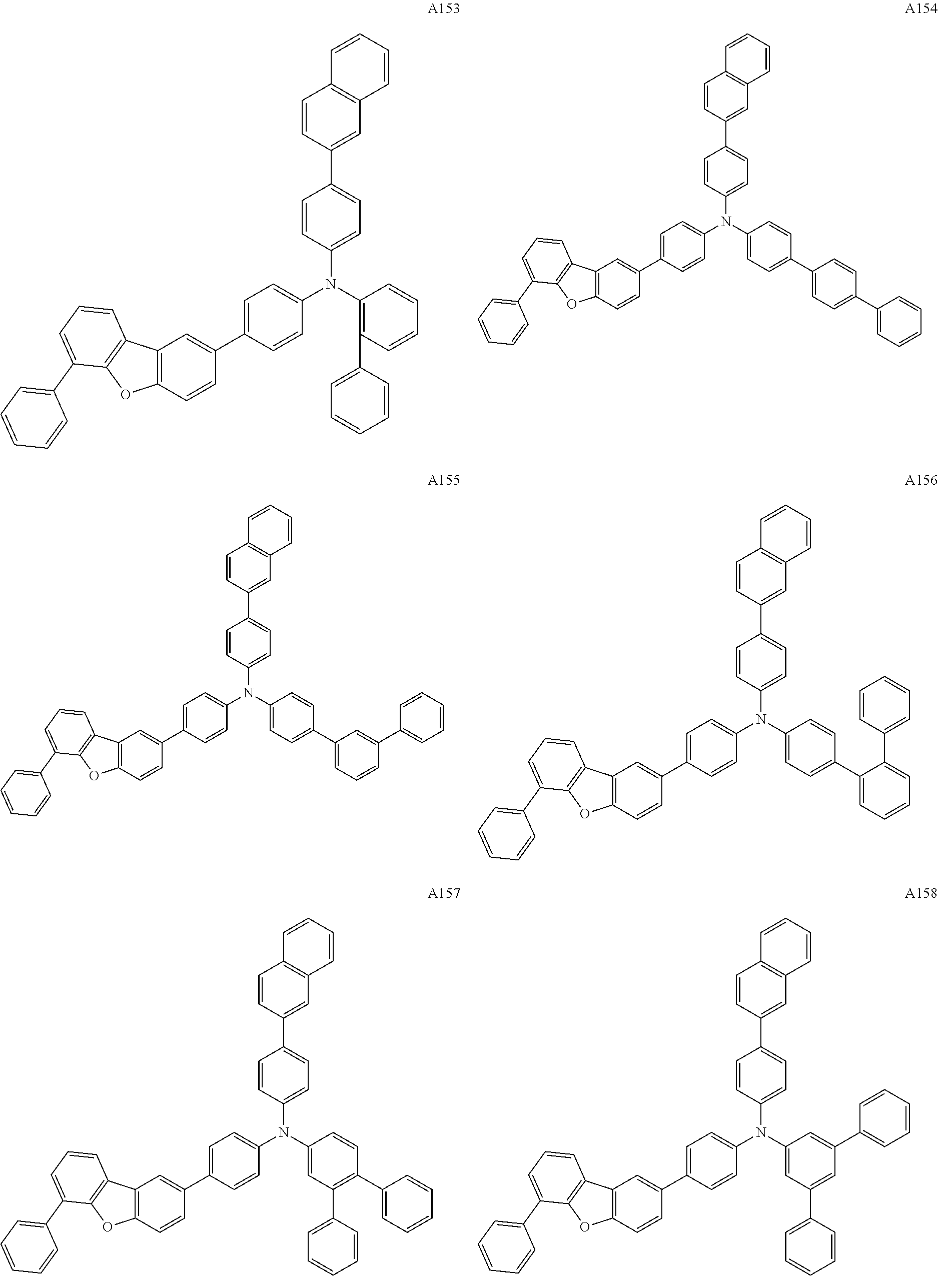

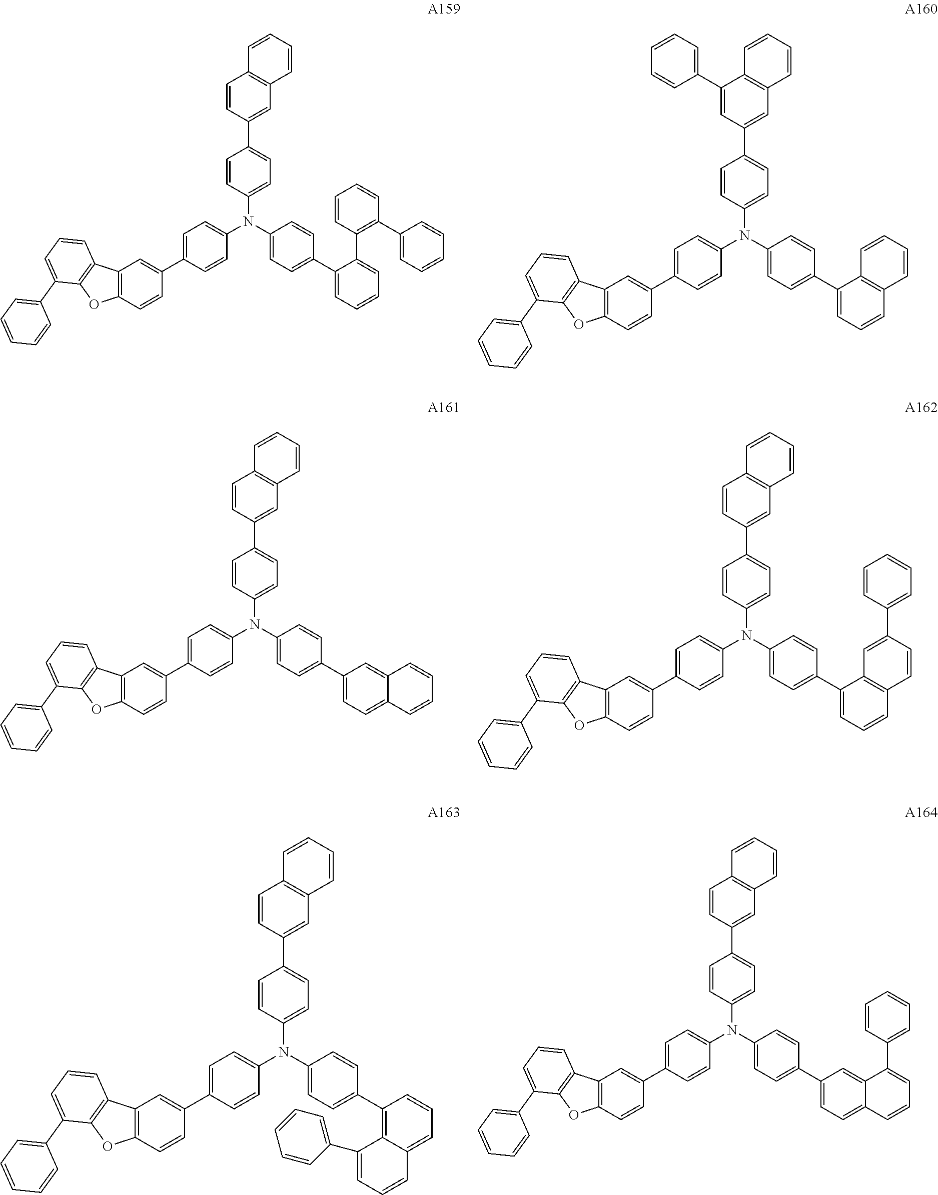

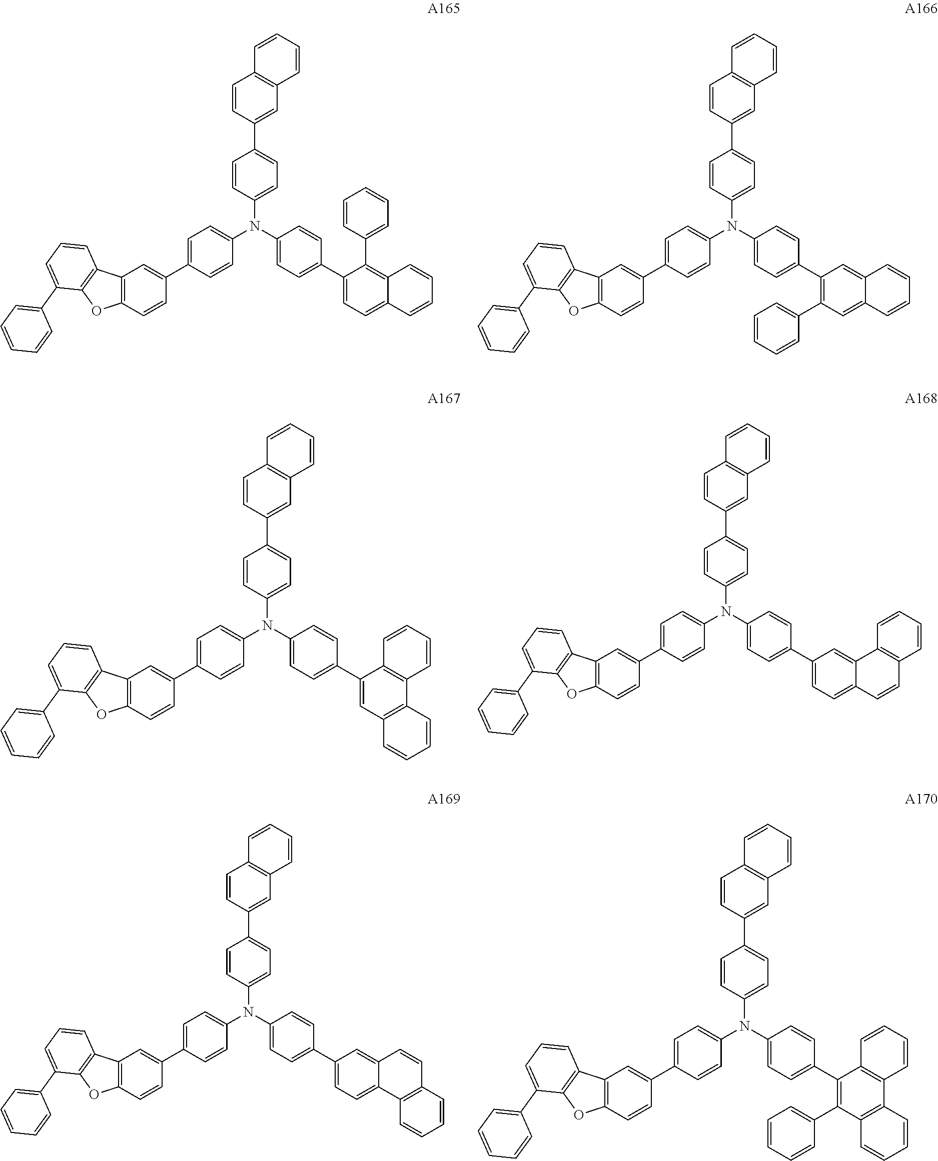

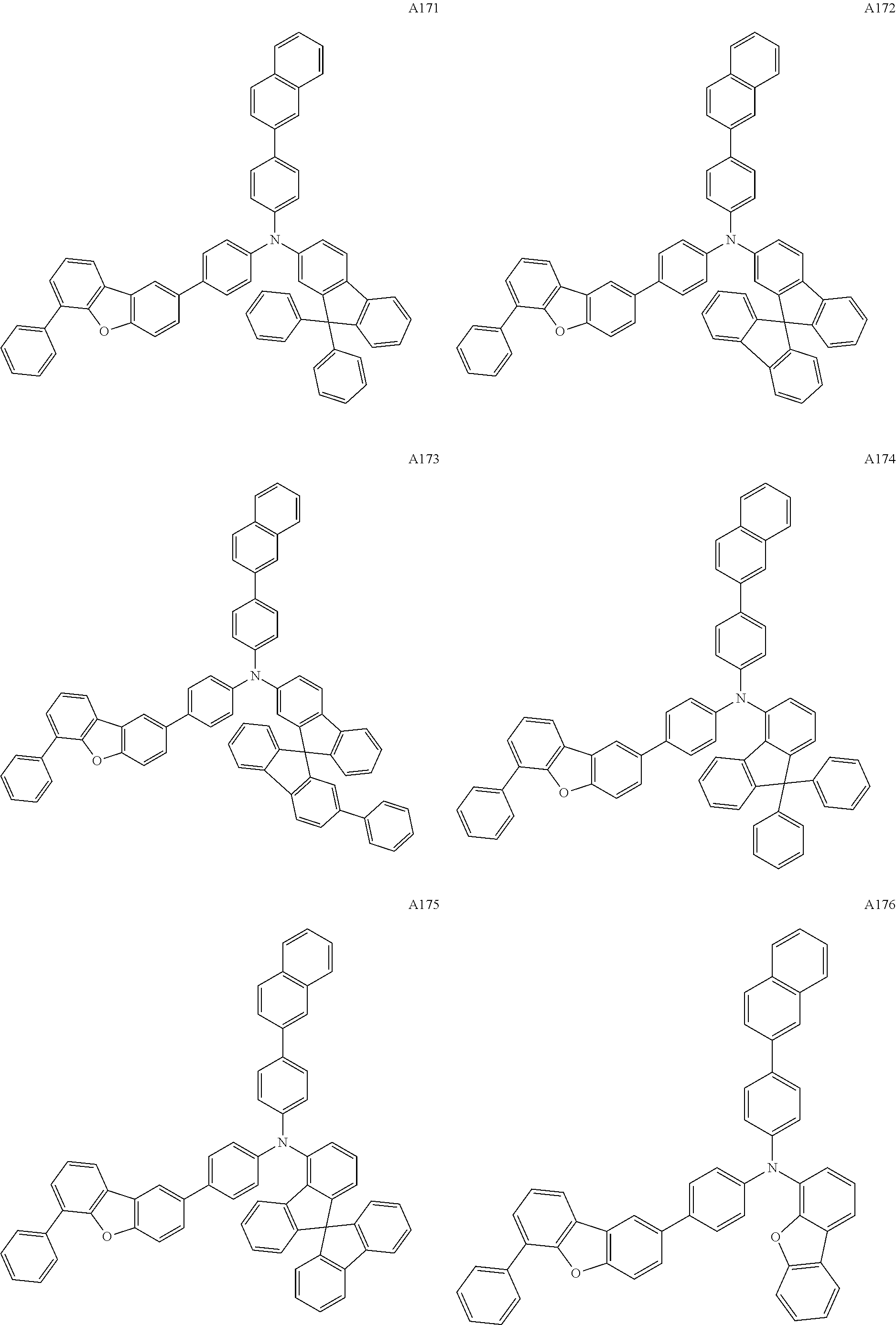

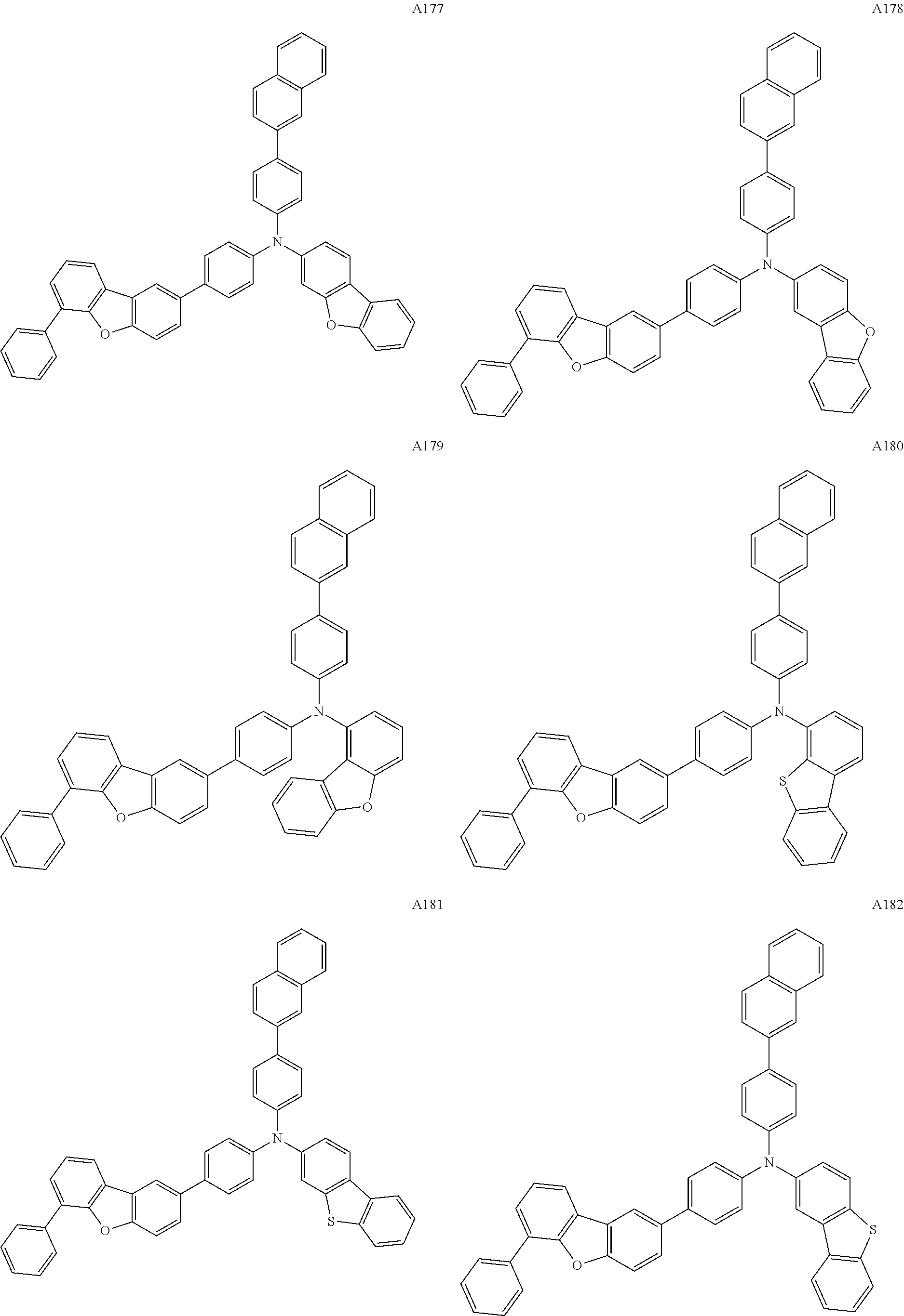

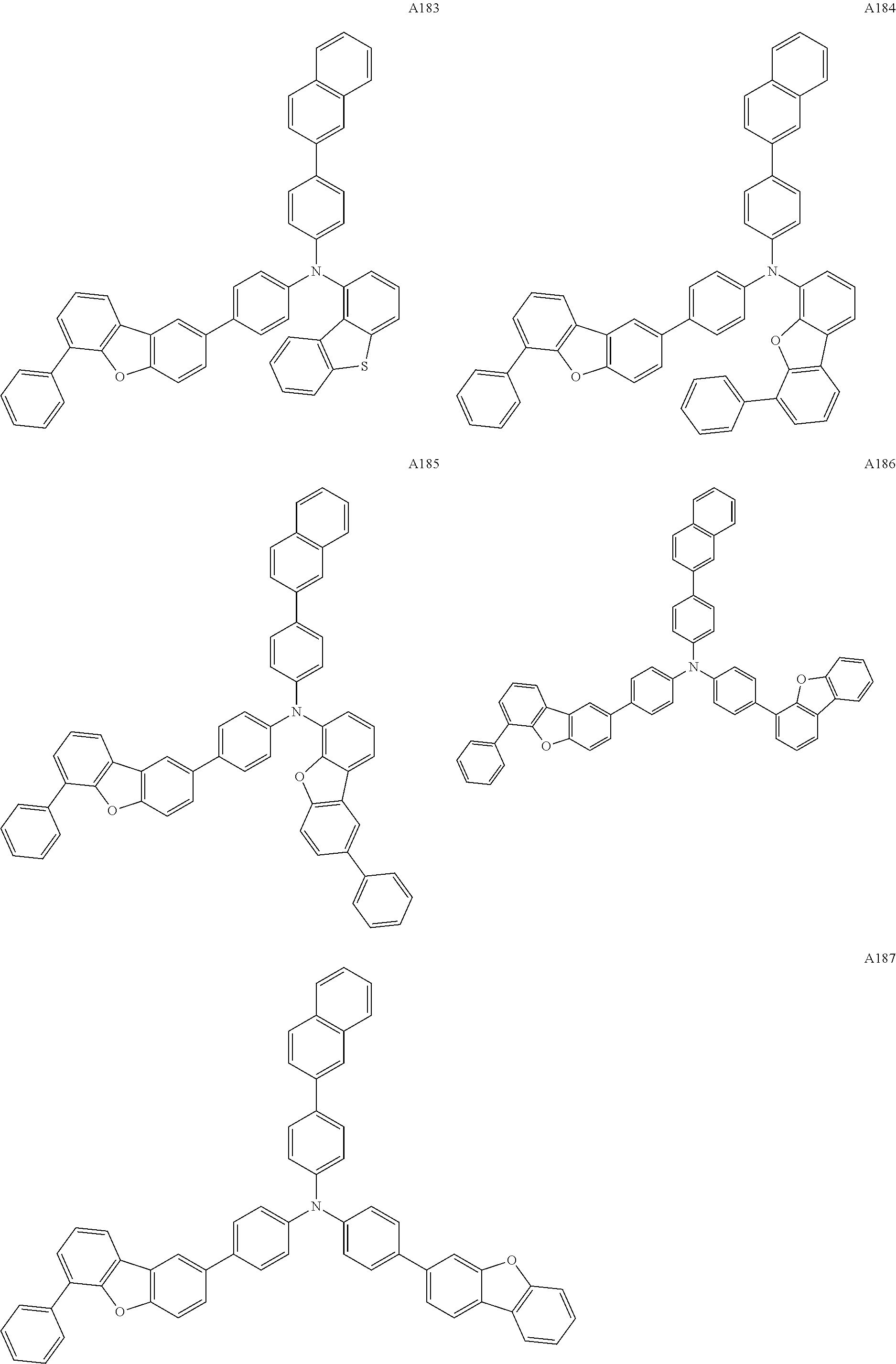

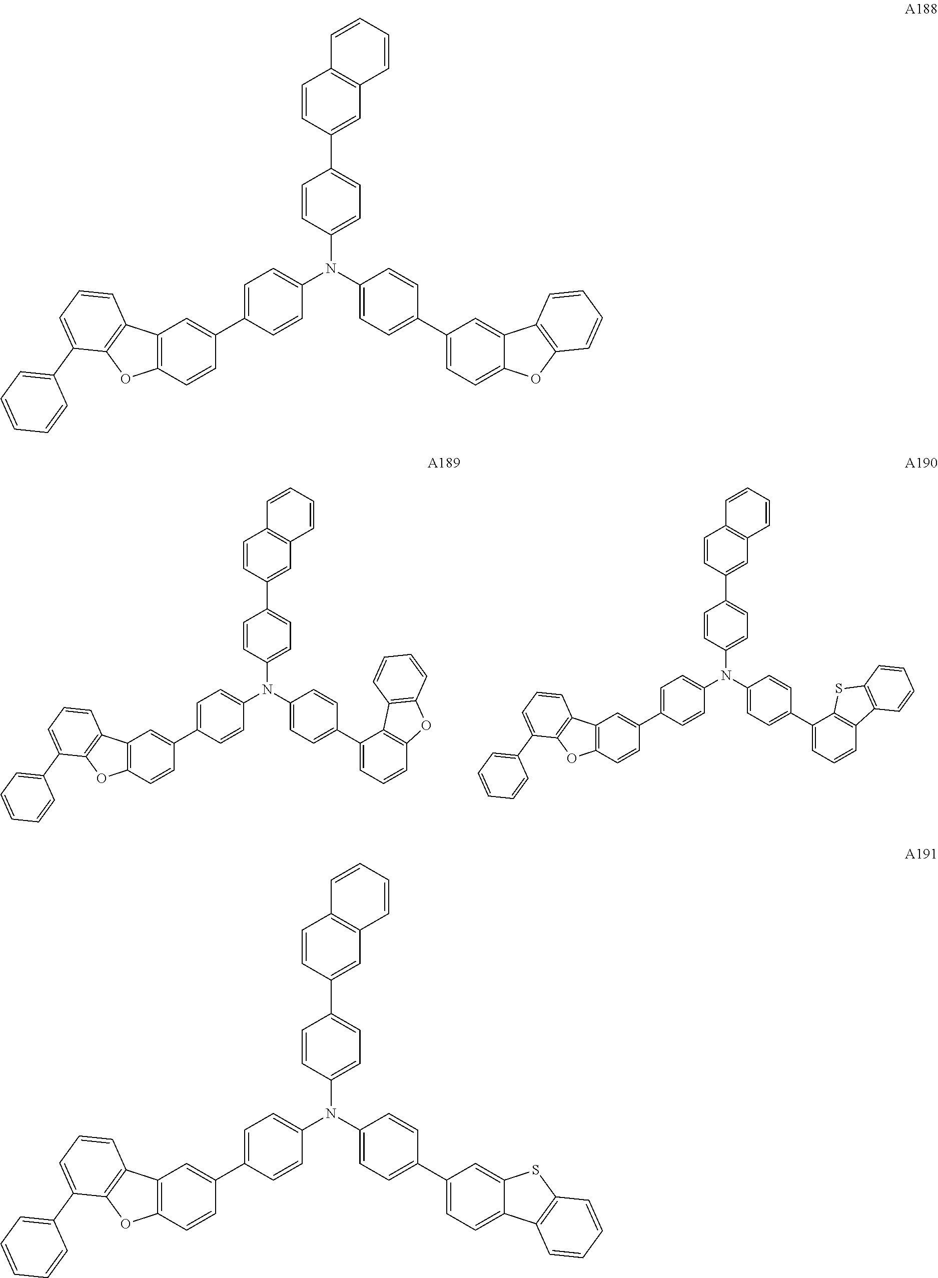 [SMILES: c1ccc(-c2cccc3c2oc2ccc(-c4ccc(N(c5ccc(-c6ccc7ccccc7c6)cc5)c5ccc(-c6ccc7oc8ccccc8c7c6)cc5)cc4)cc23)cc1 ; c1ccc(-c2cccc3c2oc2ccc(-c4ccc(N(c5ccc(-c6ccc7ccccc7c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7ccccc7c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7ccccc7c6)cc5)c5ccc(-c6ccc7c(c6)sc6ccccc67)cc5)cc4)cc23)cc1]

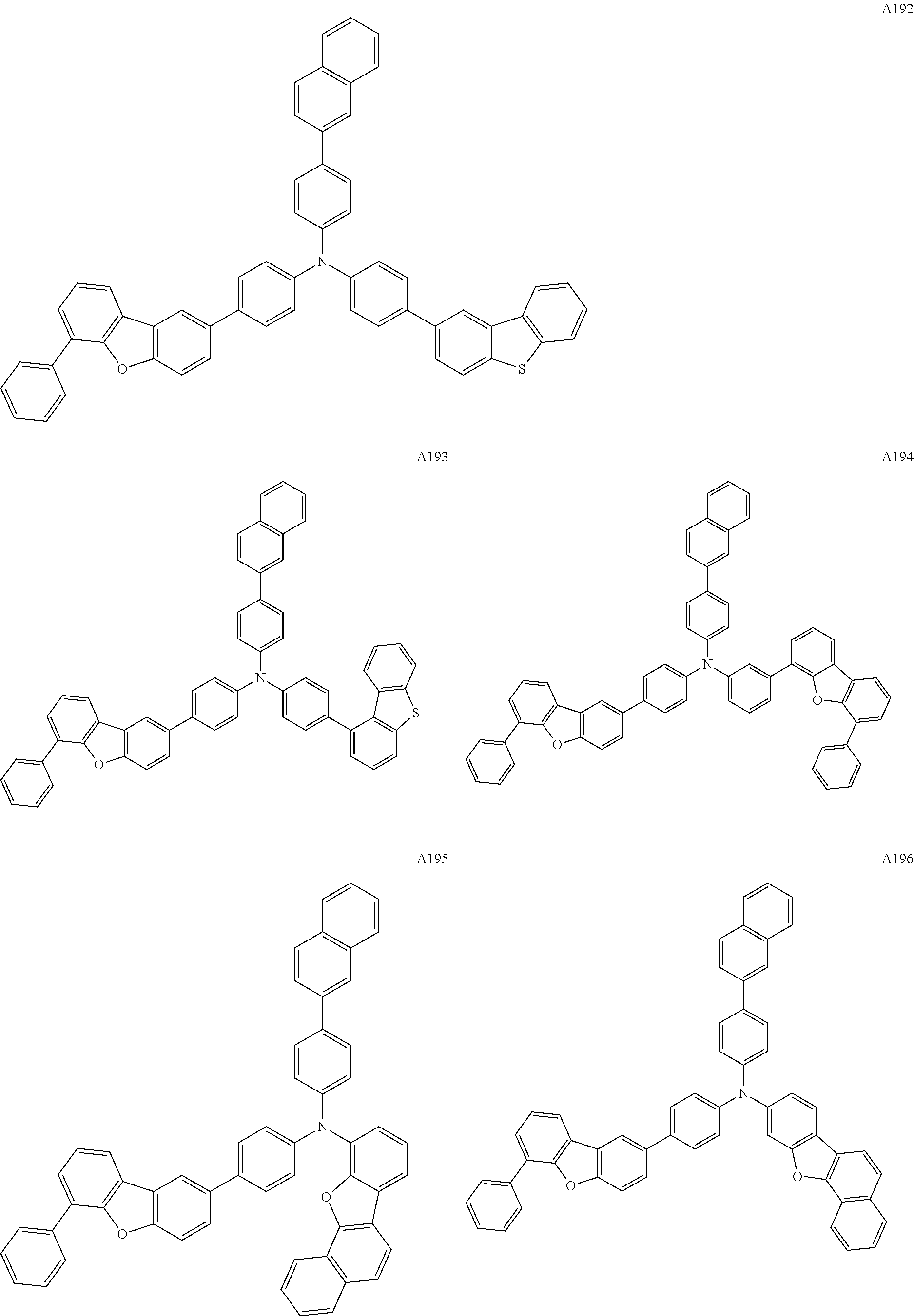

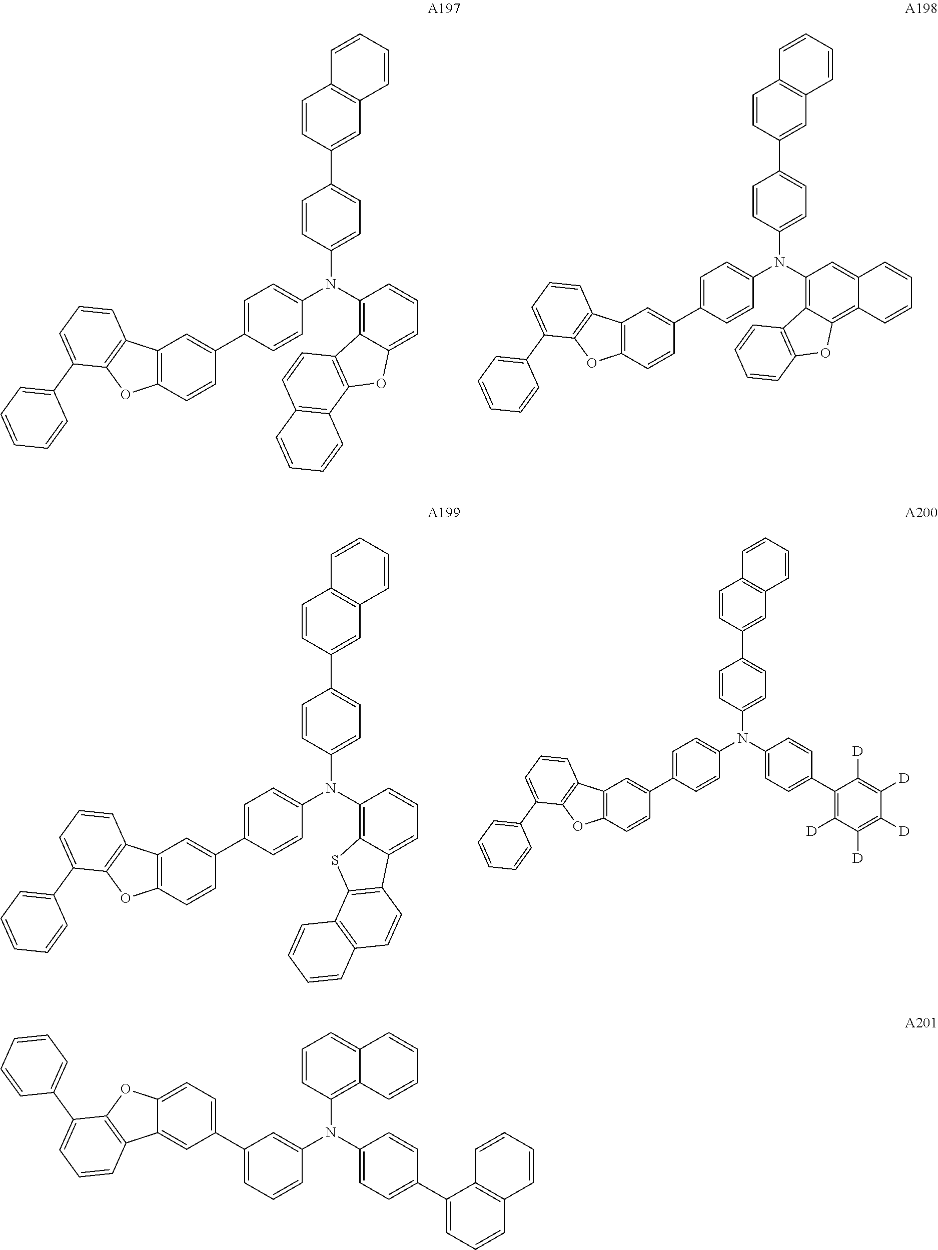

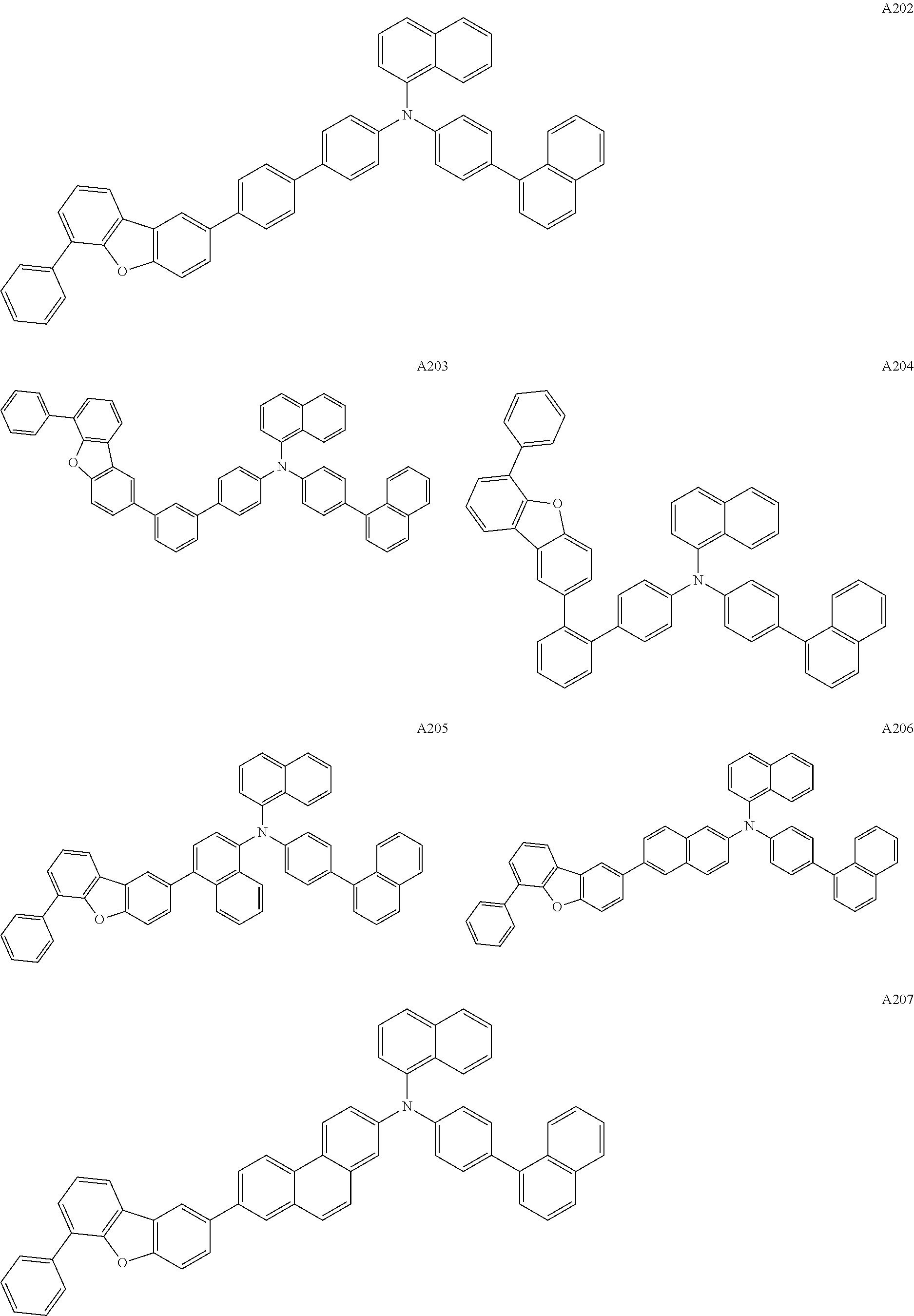

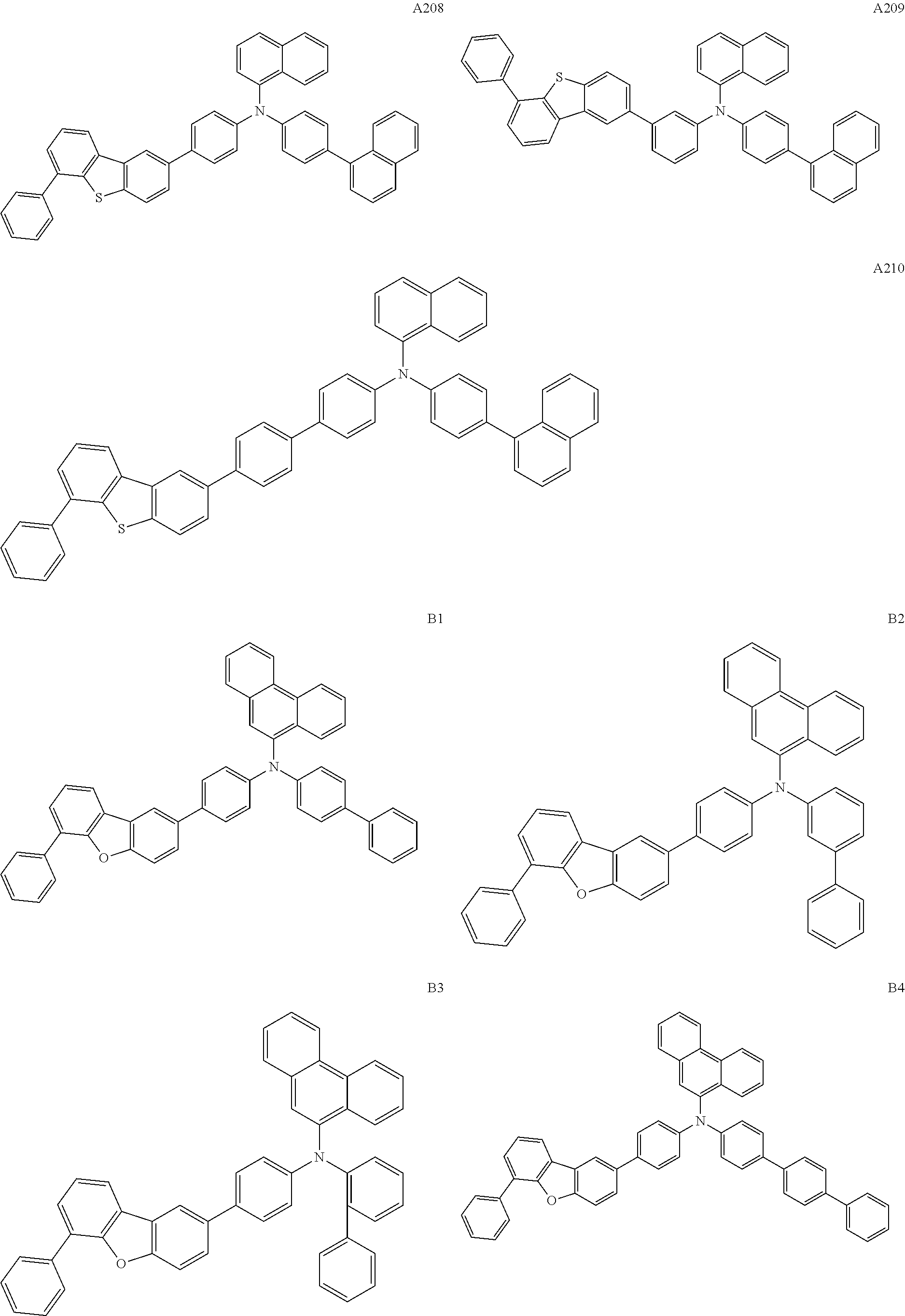

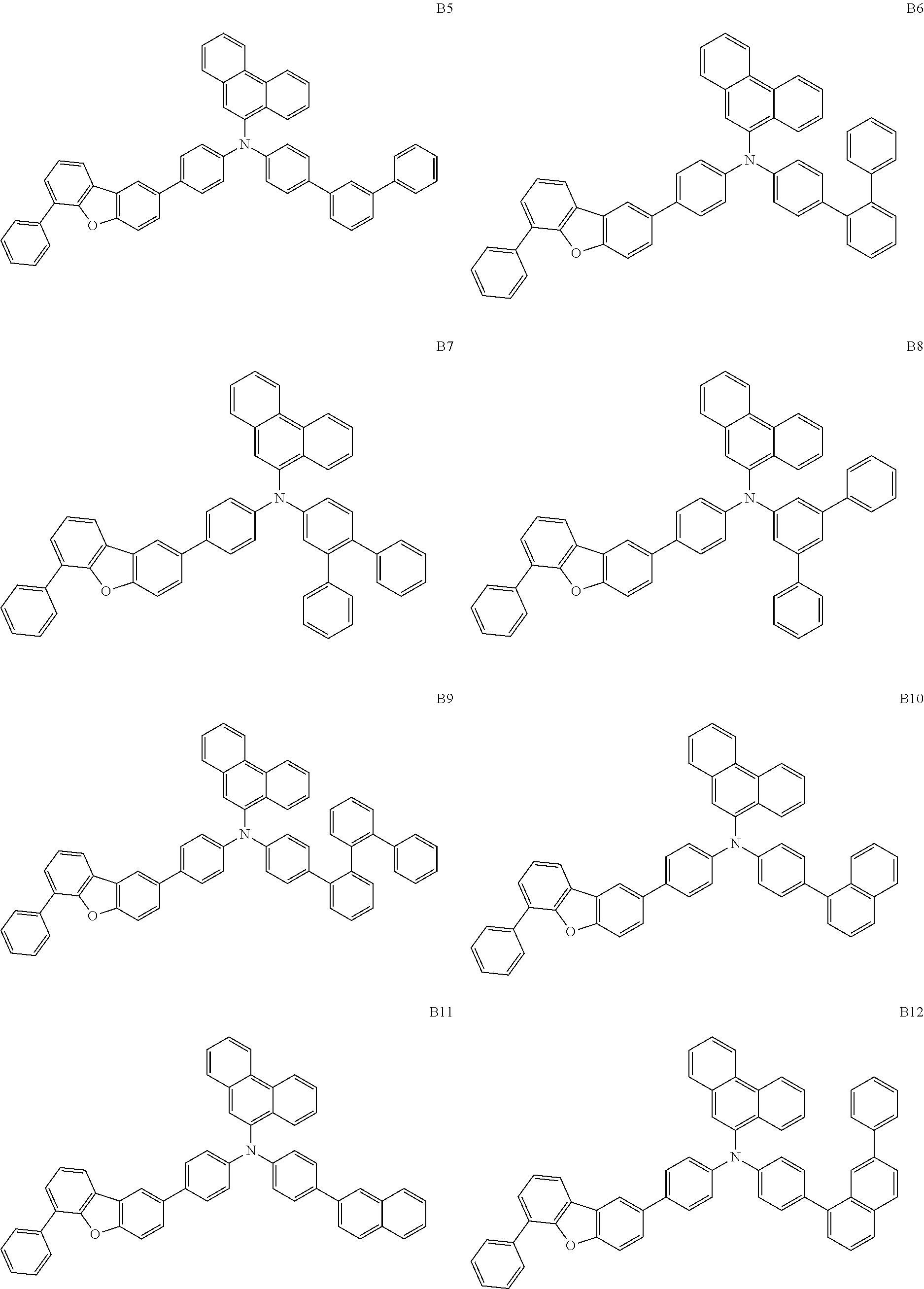

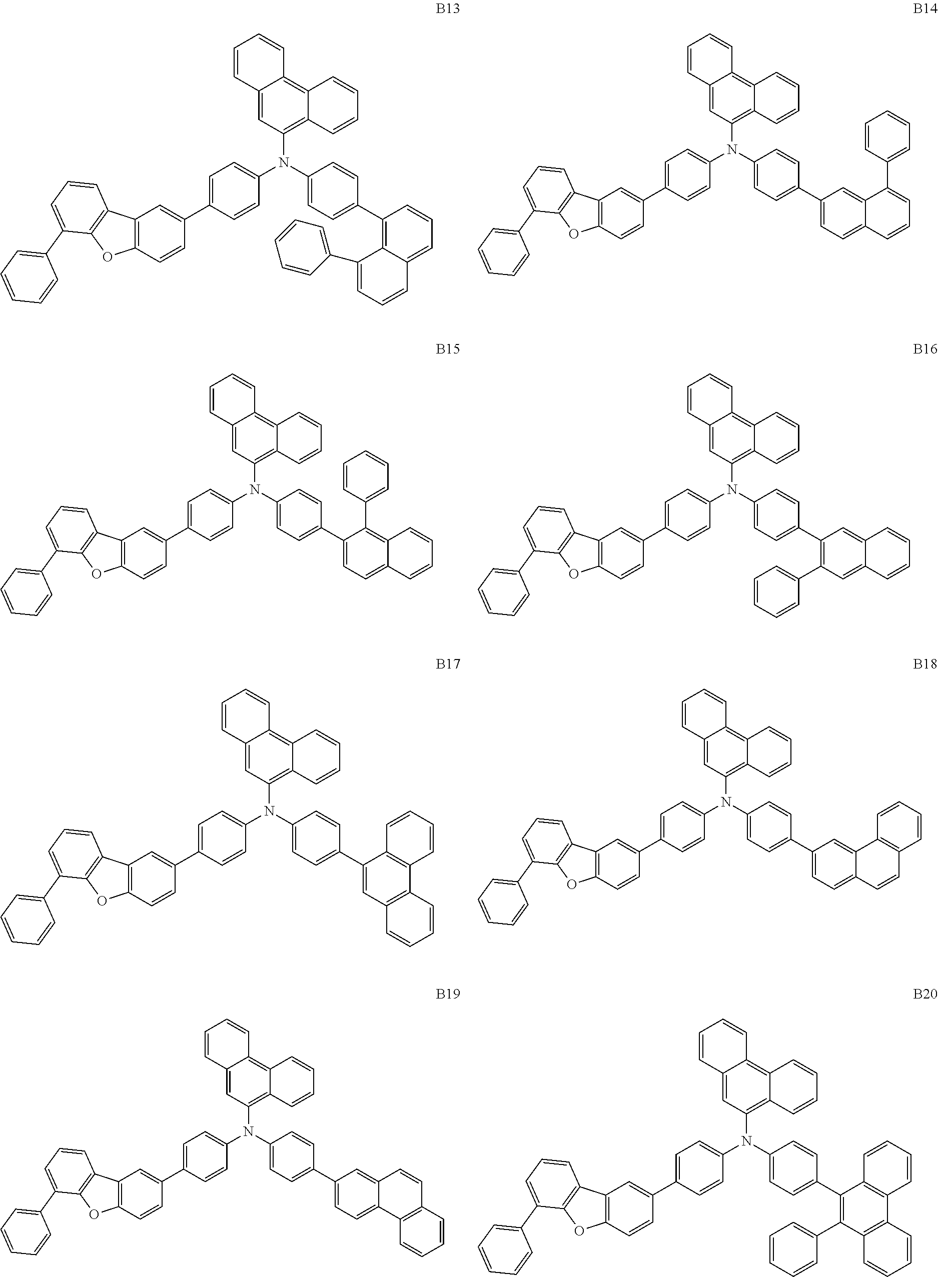

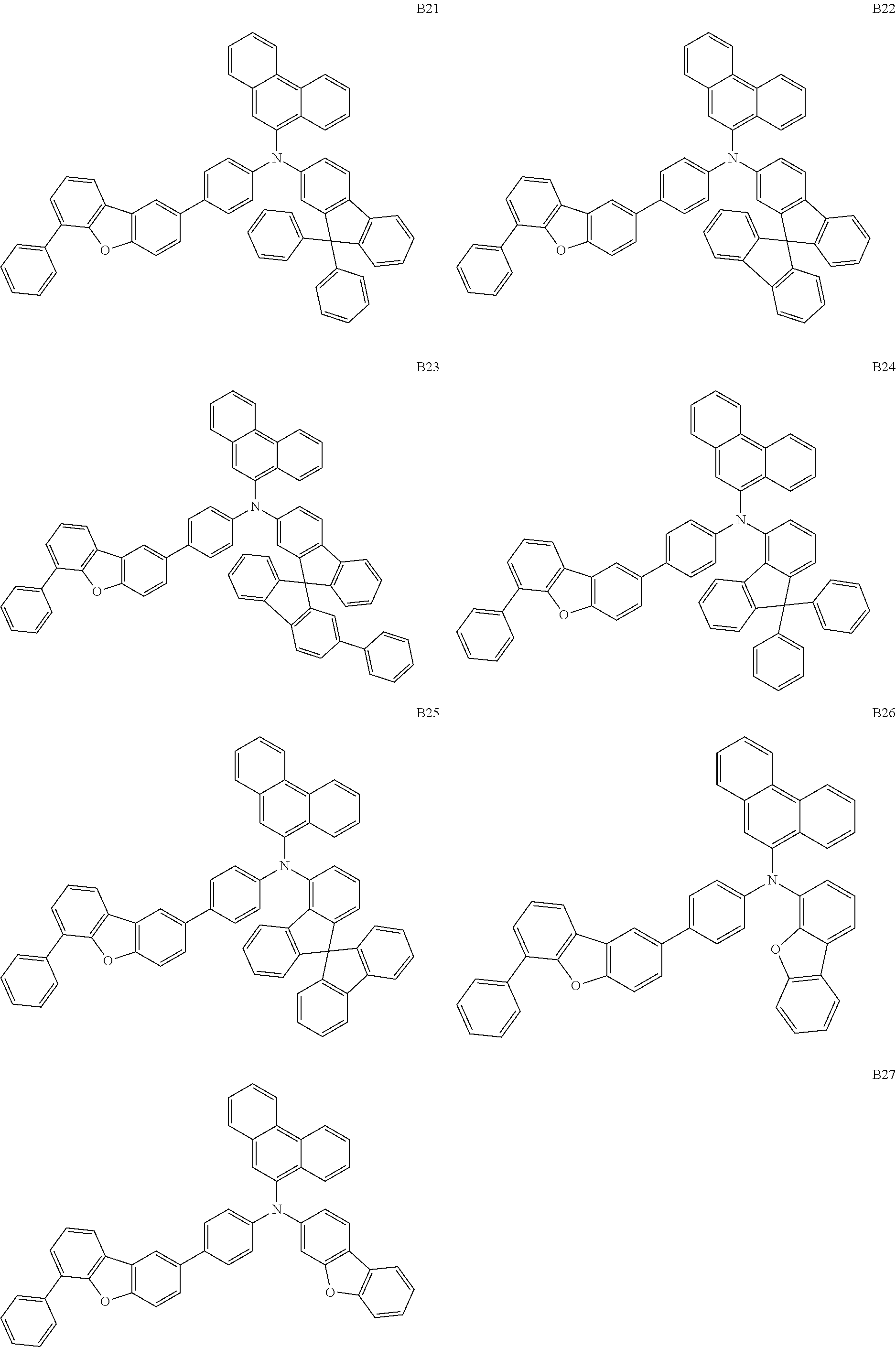

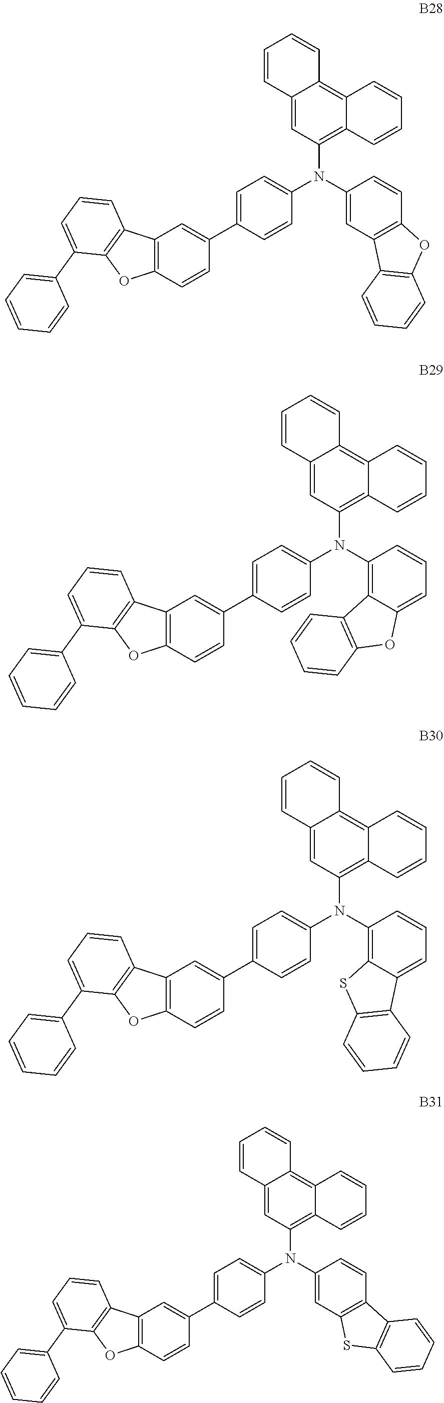 [SMILES: c1ccc(-c2cccc3c2oc2ccc(-c4ccc(N(c5ccc6oc7ccccc7c6c5)c5cc6ccccc6c6ccccc56)cc4)cc23)cc1 ; c1ccc(-c2cccc3c2oc2ccc(-c4ccc(N(c5cc6ccccc6c6ccccc56)c5cccc6oc7ccccc7c56)cc4)cc23)cc1 ; c1ccc(-c2cccc3c2oc2ccc(-c4ccc(N(c5cc6ccccc6c6ccccc56)c5cccc6c5sc5ccccc56)cc4)cc23)cc1 ; c1ccc(-c2cccc3c2oc2ccc(-c4ccc(N(c5ccc6c(c5)sc5ccccc56)c5cc6ccccc6c6ccccc56)cc4)cc23)cc1]

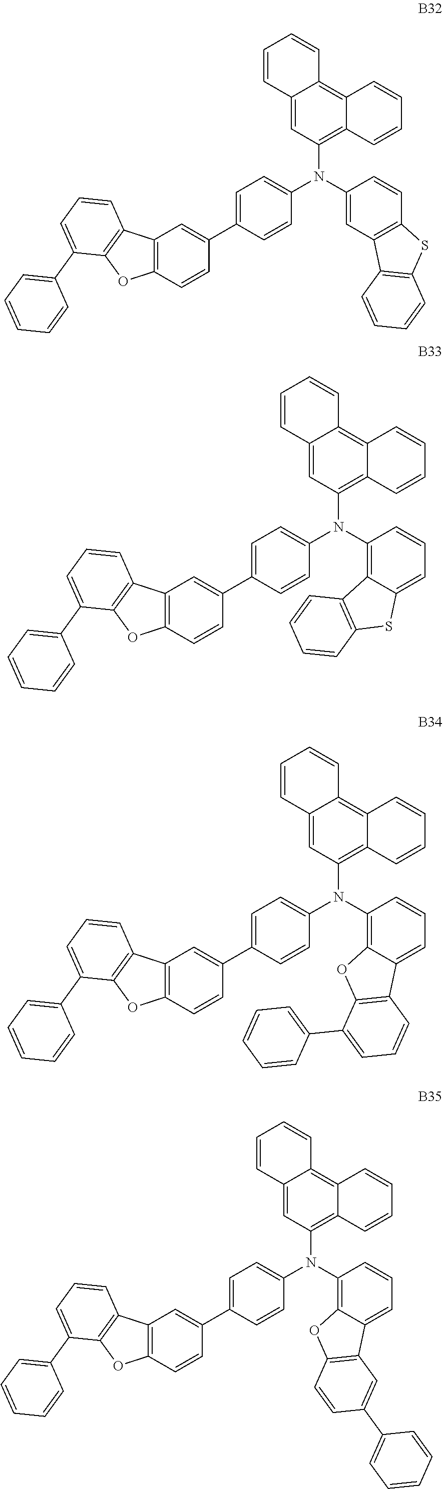 [SMILES: c1ccc(-c2cccc3c2oc2ccc(-c4ccc(N(c5ccc6sc7ccccc7c6c5)c5cc6ccccc6c6ccccc56)cc4)cc23)cc1 ; c1ccc(-c2cccc3c2oc2ccc(-c4ccc(N(c5cc6ccccc6c6ccccc56)c5cccc6sc7ccccc7c56)cc4)cc23)cc1 ; c1ccc(-c2cccc3c2oc2ccc(-c4ccc(N(c5cc6ccccc6c6ccccc56)c5cccc6c5oc5c(-c7ccccc7)cccc56)cc4)cc23)cc1 ; c1ccc(-c2ccc3oc4c(N(c5ccc(-c6ccc7oc8c(-c9ccccc9)cccc8c7c6)cc5)c5cc6ccccc6c6ccccc56)cccc4c3c2)cc1]

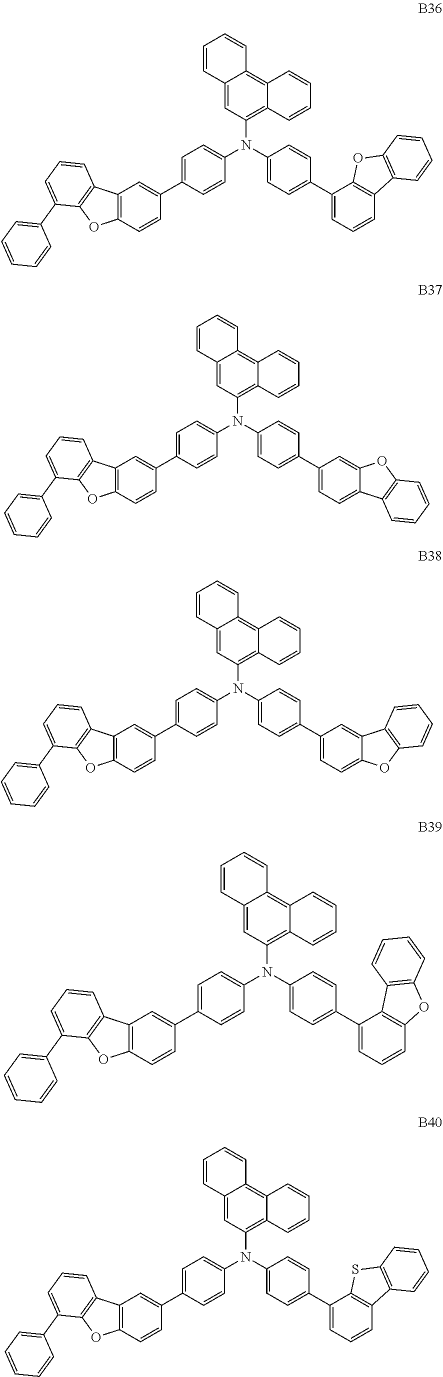 [SMILES: c1ccc(-c2cccc3c2oc2ccc(-c4ccc(N(c5ccc(-c6cccc7c6oc6ccccc67)cc5)c5cc6ccccc6c6ccccc56)cc4)cc23)cc1 ; c1ccc(-c2cccc3c2oc2ccc(-c4ccc(N(c5ccc(-c6ccc7c(c6)oc6ccccc67)cc5)c5cc6ccccc6c6ccccc56)cc4)cc23)cc1 ; c1ccc(-c2cccc3c2oc2ccc(-c4ccc(N(c5ccc(-c6ccc7oc8ccccc8c7c6)cc5)c5cc6ccccc6c6ccccc56)cc4)cc23)cc1 ; c1ccc(-c2cccc3c2oc2ccc(-c4ccc(N(c5ccc(-c6cccc7oc8ccccc8c67)cc5)c5cc6ccccc6c6ccccc56)cc4)cc23)cc1 ; c1ccc(-c2cccc3c2oc2ccc(-c4ccc(N(c5ccc(-c6cccc7c6sc6ccccc67)cc5)c5cc6ccccc6c6ccccc56)cc4)cc23)cc1]

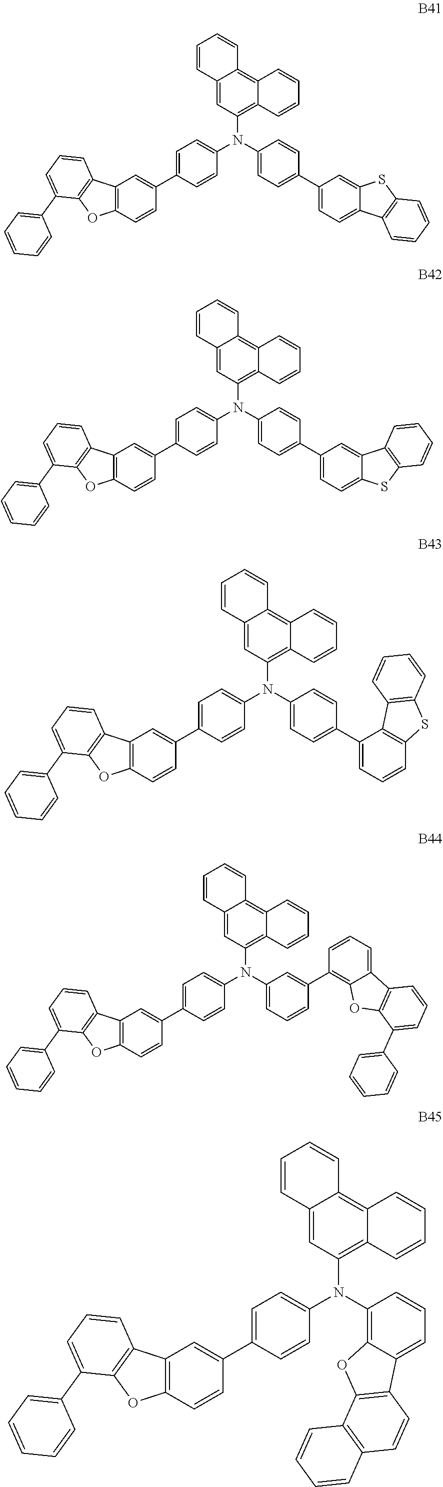 [SMILES: c1ccc(-c2cccc3c2oc2ccc(-c4ccc(N(c5ccc(-c6ccc7c(c6)sc6ccccc67)cc5)c5cc6ccccc6c6ccccc56)cc4)cc23)cc1 ; c1ccc(-c2cccc3c2oc2ccc(-c4ccc(N(c5ccc(-c6ccc7sc8ccccc8c7c6)cc5)c5cc6ccccc6c6ccccc56)cc4)cc23)cc1 ; c1ccc(-c2cccc3c2oc2ccc(-c4ccc(N(c5ccc(-c6cccc7sc8ccccc8c67)cc5)c5cc6ccccc6c6ccccc56)cc4)cc23)cc1 ; c1ccc(-c2cccc3c2oc2ccc(-c4ccc(N(c5cccc(-c6cccc7c6oc6c(-c8ccccc8)cccc67)c5)c5cc6ccccc6c6ccccc56)cc4)cc23)cc1 ; c1ccc(-c2cccc3c2oc2ccc(-c4ccc(N(c5cc6ccccc6c6ccccc56)c5cccc6c5oc5c7ccccc7ccc65)cc4)cc23)cc1]

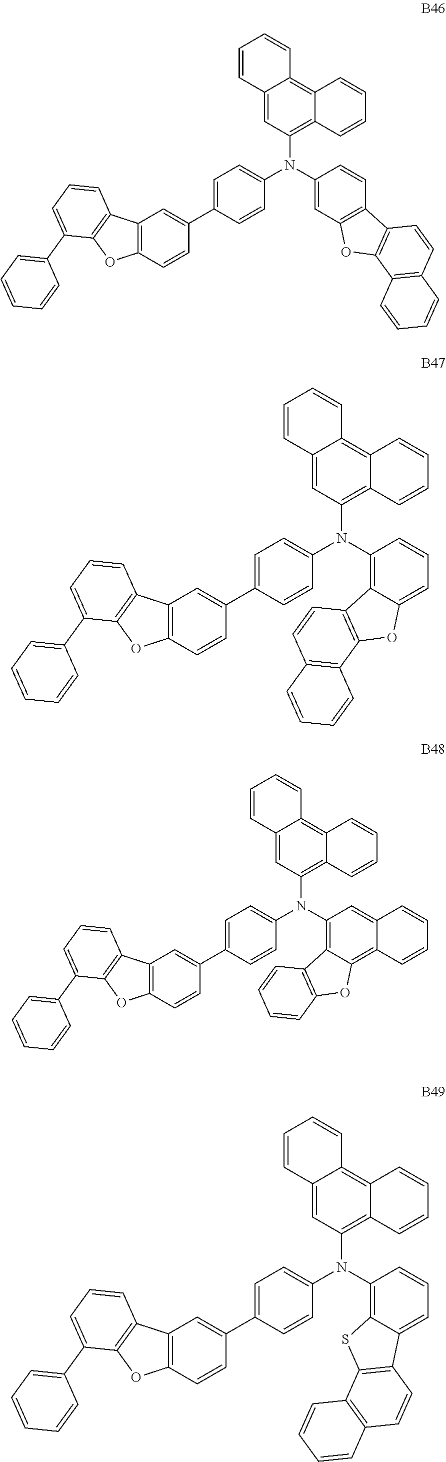 [SMILES: c1ccc(-c2cccc3c2oc2ccc(-c4ccc(N(c5ccc6c(c5)oc5c7ccccc7ccc65)c5cc6ccccc6c6ccccc56)cc4)cc23)cc1 ; c1ccc(-c2cccc3c2oc2ccc(-c4ccc(N(c5cc6ccccc6c6ccccc56)c5cccc6oc7c8ccccc8ccc7c56)cc4)cc23)cc1 ; c1ccc(-c2cccc3c2oc2ccc(-c4ccc(N(c5cc6ccccc6c6ccccc56)c5cc6ccccc6c6oc7ccccc7c56)cc4)cc23)cc1 ; c1ccc(-c2cccc3c2oc2ccc(-c4ccc(N(c5cc6ccccc6c6ccccc56)c5cccc6c5sc5c7ccccc7ccc65)cc4)cc23)cc1]

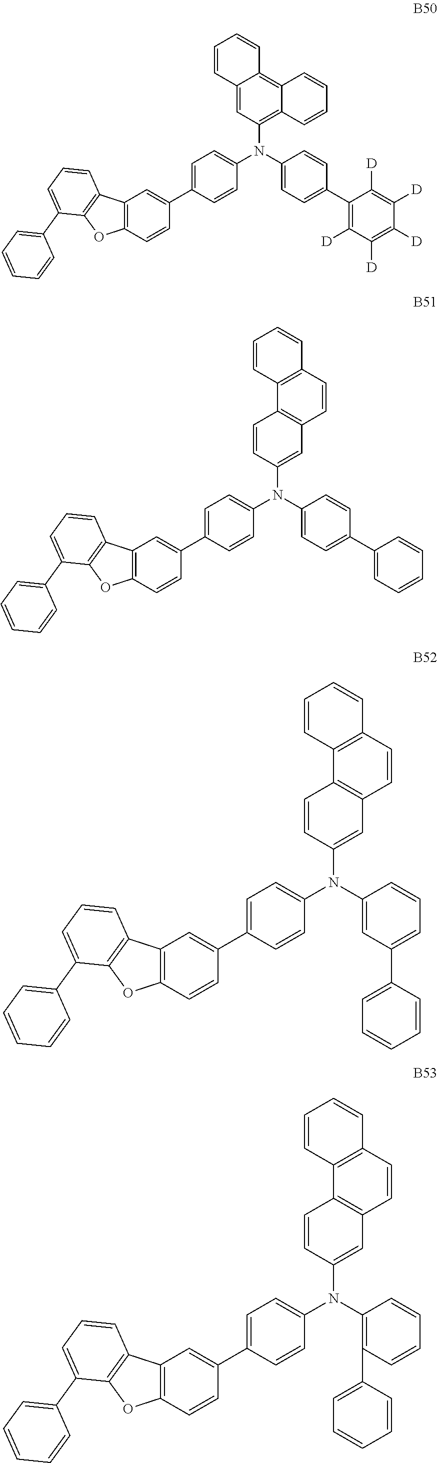 [SMILES: [2H]c1c([2H])c([2H])c(-c2ccc(N(c3ccc(-c4ccc5oc6c(-c7ccccc7)cccc6c5c4)cc3)c3cc4ccccc4c4ccccc34)cc2)c([2H])c1[2H] ; c1ccc(-c2ccc(N(c3ccc(-c4ccc5oc6c(-c7ccccc7)cccc6c5c4)cc3)c3ccc4c(ccc5ccccc54)c3)cc2)cc1 ; c1ccc(-c2cccc(N(c3ccc(-c4ccc5oc6c(-c7ccccc7)cccc6c5c4)cc3)c3ccc4c(ccc5ccccc54)c3)c2)cc1 ; c1ccc(-c2ccccc2N(c2ccc(-c3ccc4oc5c(-c6ccccc6)cccc5c4c3)cc2)c2ccc3c(ccc4ccccc43)c2)cc1]

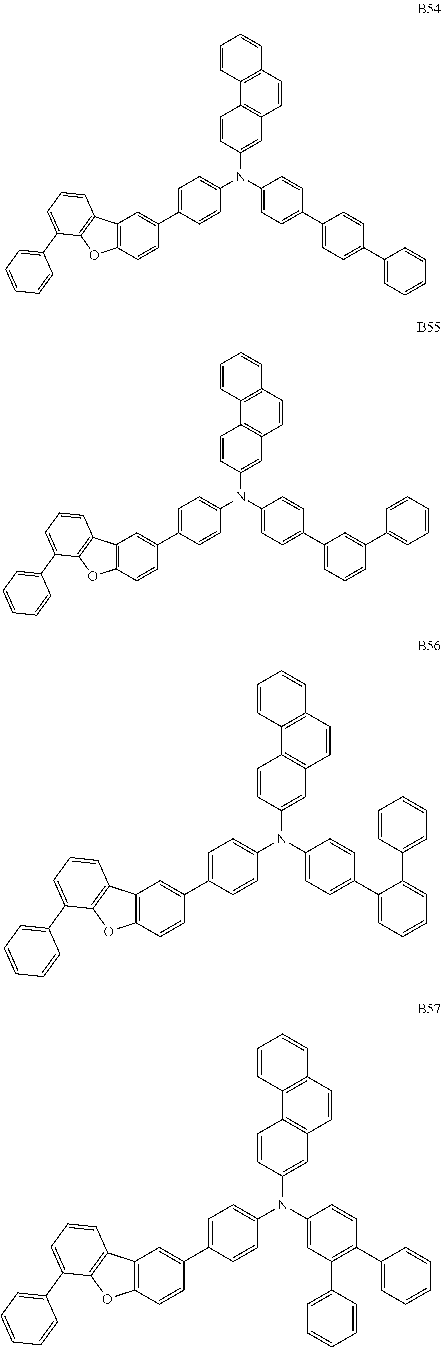 [SMILES: c1ccc(-c2ccc(-c3ccc(N(c4ccc(-c5ccc6oc7c(-c8ccccc8)cccc7c6c5)cc4)c4ccc5c(ccc6ccccc65)c4)cc3)cc2)cc1 ; c1ccc(-c2cccc(-c3ccc(N(c4ccc(-c5ccc6oc7c(-c8ccccc8)cccc7c6c5)cc4)c4ccc5c(ccc6ccccc65)c4)cc3)c2)cc1 ; c1ccc(-c2ccccc2-c2ccc(N(c3ccc(-c4ccc5oc6c(-c7ccccc7)cccc6c5c4)cc3)c3ccc4c(ccc5ccccc54)c3)cc2)cc1 ; c1ccc(-c2ccc(N(c3ccc(-c4ccc5oc6c(-c7ccccc7)cccc6c5c4)cc3)c3ccc4c(ccc5ccccc54)c3)cc2-c2ccccc2)cc1]

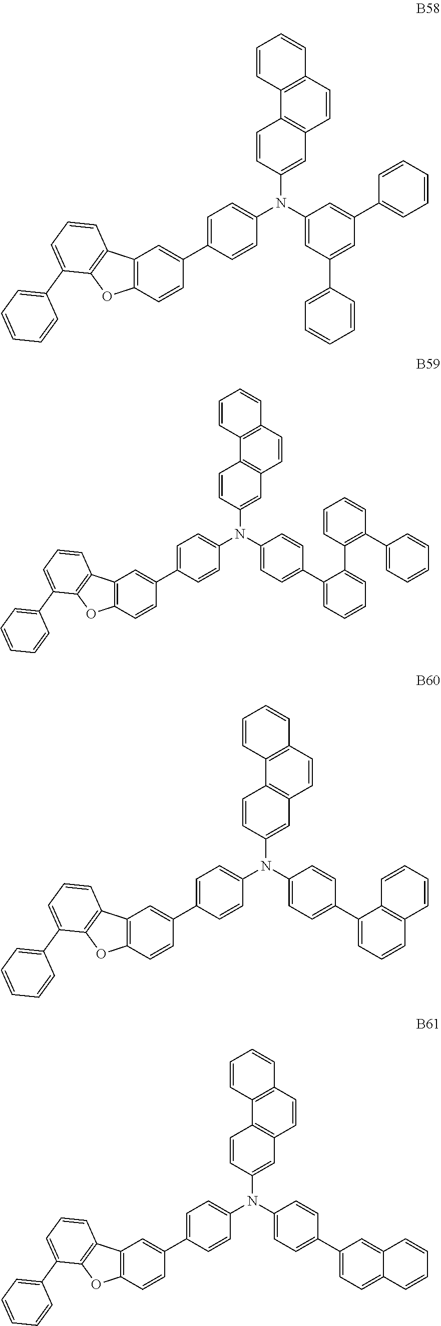 [SMILES: c1ccc(-c2cc(-c3ccccc3)cc(N(c3ccc(-c4ccc5oc6c(-c7ccccc7)cccc6c5c4)cc3)c3ccc4c(ccc5ccccc54)c3)c2)cc1 ; c1ccc(-c2ccccc2-c2ccccc2-c2ccc(N(c3ccc(-c4ccc5oc6c(-c7ccccc7)cccc6c5c4)cc3)c3ccc4c(ccc5ccccc54)c3)cc2)cc1 ; c1ccc(-c2cccc3c2oc2ccc(-c4ccc(N(c5ccc(-c6cccc7ccccc67)cc5)c5ccc6c(ccc7ccccc76)c5)cc4)cc23)cc1 ; c1ccc(-c2cccc3c2oc2ccc(-c4ccc(N(c5ccc(-c6ccc7ccccc7c6)cc5)c5ccc6c(ccc7ccccc76)c5)cc4)cc23)cc1]

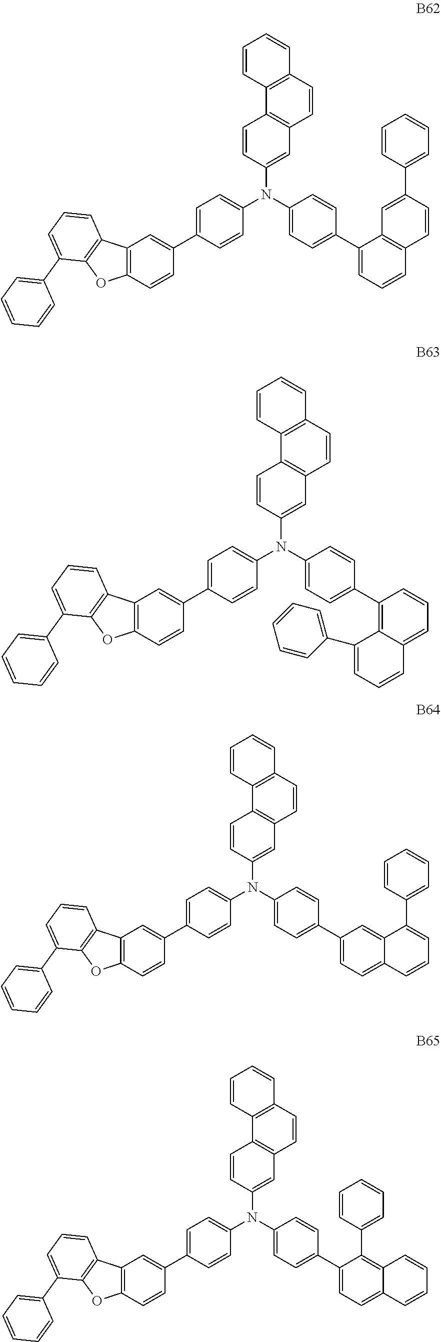 [SMILES: c1ccc(-c2ccc3cccc(-c4ccc(N(c5ccc(-c6ccc7oc8c(-c9ccccc9)cccc8c7c6)cc5)c5ccc6c(ccc7ccccc76)c5)cc4)c3c2)cc1 ; c1ccc(-c2cccc3c2oc2ccc(-c4ccc(N(c5ccc(-c6cccc7cccc(-c8ccccc8)c67)cc5)c5ccc6c(ccc7ccccc76)c5)cc4)cc23)cc1 ; c1ccc(-c2cccc3ccc(-c4ccc(N(c5ccc(-c6ccc7oc8c(-c9ccccc9)cccc8c7c6)cc5)c5ccc6c(ccc7ccccc76)c5)cc4)cc23)cc1 ; c1ccc(-c2c(-c3ccc(N(c4ccc(-c5ccc6oc7c(-c8ccccc8)cccc7c6c5)cc4)c4ccc5c(ccc6ccccc65)c4)cc3)ccc3ccccc23)cc1]

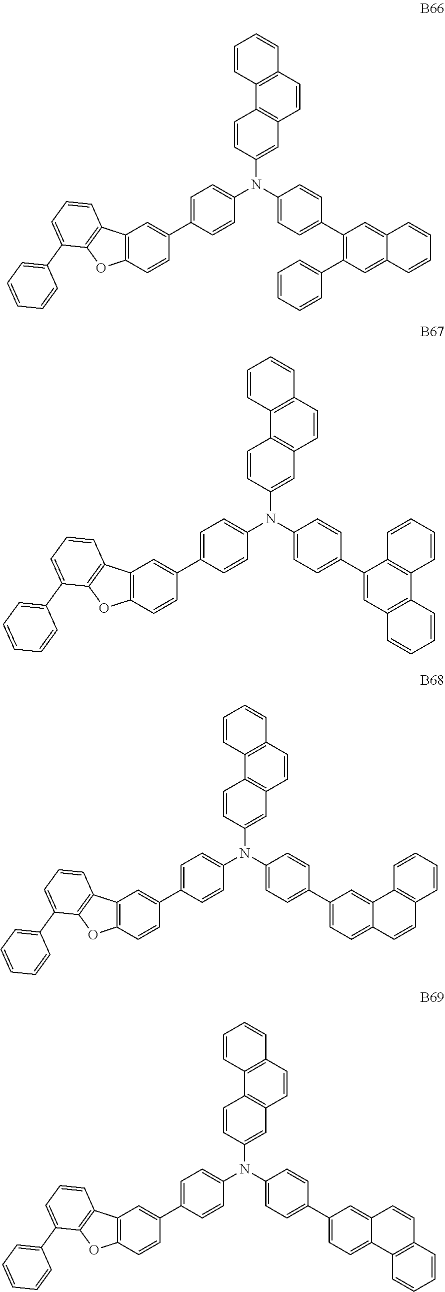 [SMILES: c1ccc(-c2cc3ccccc3cc2-c2ccc(N(c3ccc(-c4ccc5oc6c(-c7ccccc7)cccc6c5c4)cc3)c3ccc4c(ccc5ccccc54)c3)cc2)cc1 ; c1ccc(-c2cccc3c2oc2ccc(-c4ccc(N(c5ccc(-c6cc7ccccc7c7ccccc67)cc5)c5ccc6c(ccc7ccccc76)c5)cc4)cc23)cc1 ; c1ccc(-c2cccc3c2oc2ccc(-c4ccc(N(c5ccc(-c6ccc7ccc8ccccc8c7c6)cc5)c5ccc6c(ccc7ccccc76)c5)cc4)cc23)cc1 ; c1ccc(-c2cccc3c2oc2ccc(-c4ccc(N(c5ccc(-c6ccc7c(ccc8ccccc87)c6)cc5)c5ccc6c(ccc7ccccc76)c5)cc4)cc23)cc1]

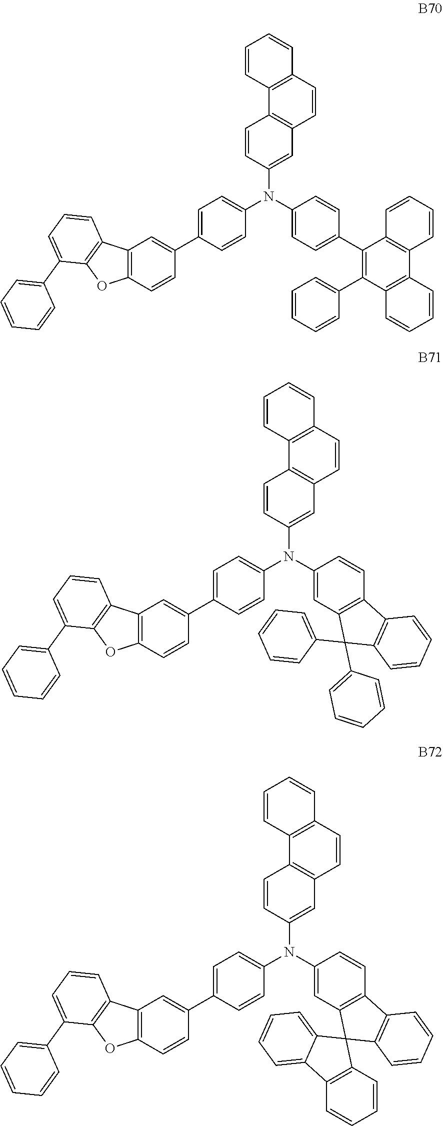 [SMILES: c1ccc(-c2c(-c3ccc(N(c4ccc(-c5ccc6oc7c(-c8ccccc8)cccc7c6c5)cc4)c4ccc5c(ccc6ccccc65)c4)cc3)c3ccccc3c3ccccc23)cc1 ; c1ccc(-c2cccc3c2oc2ccc(-c4ccc(N(c5ccc6c(c5)C(c5ccccc5)(c5ccccc5)c5ccccc5-6)c5ccc6c(ccc7ccccc76)c5)cc4)cc23)cc1 ; c1ccc(-c2cccc3c2oc2ccc(-c4ccc(N(c5ccc6c(c5)C5(c7ccccc7-c7ccccc75)c5ccccc5-6)c5ccc6c(ccc7ccccc76)c5)cc4)cc23)cc1]

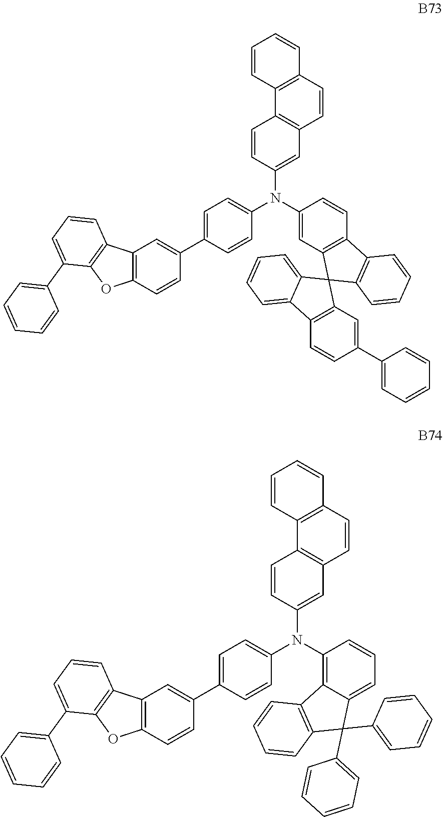 [SMILES: c1ccc(-c2ccc3c(c2)C2(c4ccccc4-3)c3ccccc3-c3ccc(N(c4ccc(-c5ccc6oc7c(-c8ccccc8)cccc7c6c5)cc4)c4ccc5c(ccc6ccccc65)c4)cc32)cc1 ; c1ccc(-c2cccc3c2oc2ccc(-c4ccc(N(c5ccc6c(ccc7ccccc76)c5)c5cccc6c5-c5ccccc5C6(c5ccccc5)c5ccccc5)cc4)cc23)cc1]

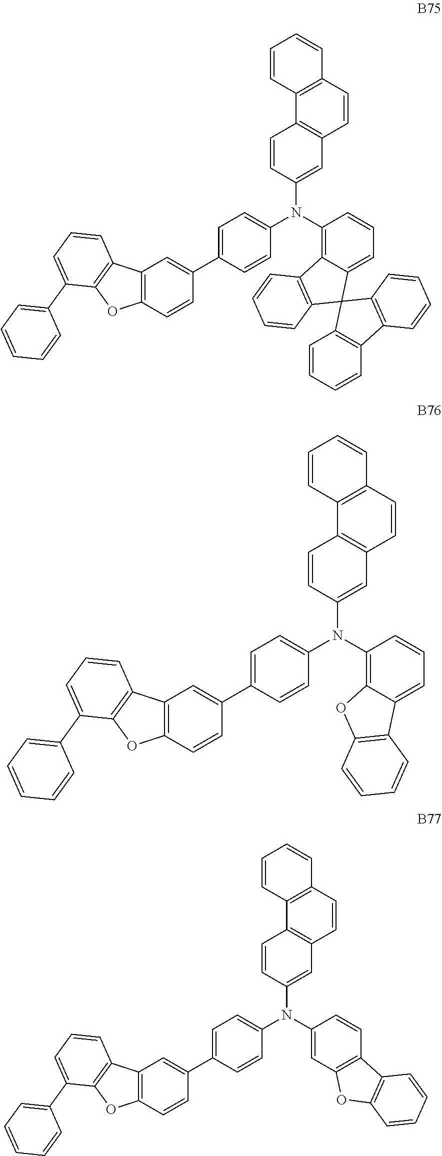 [SMILES: c1ccc(-c2cccc3c2oc2ccc(-c4ccc(N(c5ccc6c(ccc7ccccc76)c5)c5cccc6c5-c5ccccc5C65c6ccccc6-c6ccccc65)cc4)cc23)cc1 ; c1ccc(-c2cccc3c2oc2ccc(-c4ccc(N(c5ccc6c(ccc7ccccc76)c5)c5cccc6c5oc5ccccc56)cc4)cc23)cc1 ; c1ccc(-c2cccc3c2oc2ccc(-c4ccc(N(c5ccc6c(ccc7ccccc76)c5)c5ccc6c(c5)oc5ccccc56)cc4)cc23)cc1]

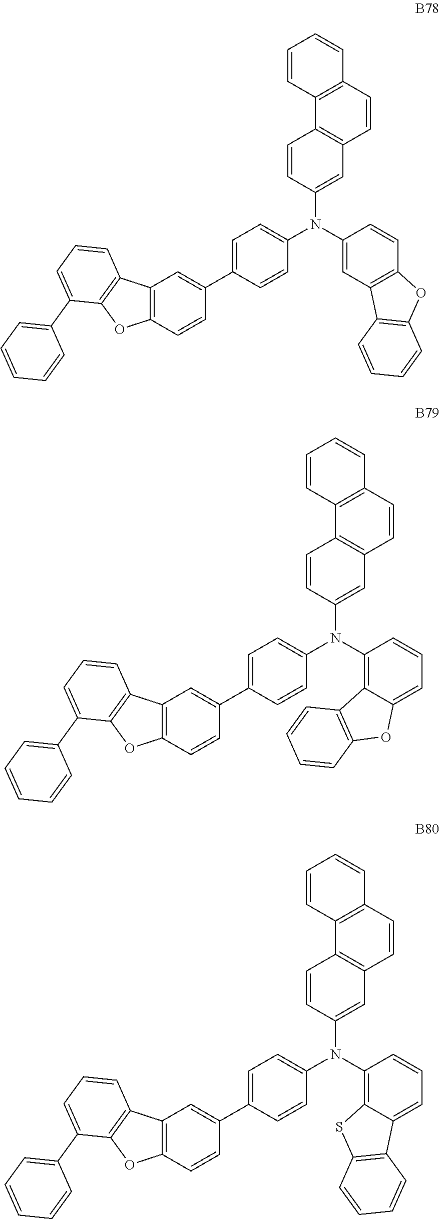 [SMILES: c1ccc(-c2cccc3c2oc2ccc(-c4ccc(N(c5ccc6c(ccc7ccccc76)c5)c5ccc6oc7ccccc7c6c5)cc4)cc23)cc1 ; c1ccc(-c2cccc3c2oc2ccc(-c4ccc(N(c5ccc6c(ccc7ccccc76)c5)c5cccc6oc7ccccc7c56)cc4)cc23)cc1 ; c1ccc(-c2cccc3c2oc2ccc(-c4ccc(N(c5ccc6c(ccc7ccccc76)c5)c5cccc6c5sc5ccccc56)cc4)cc23)cc1]

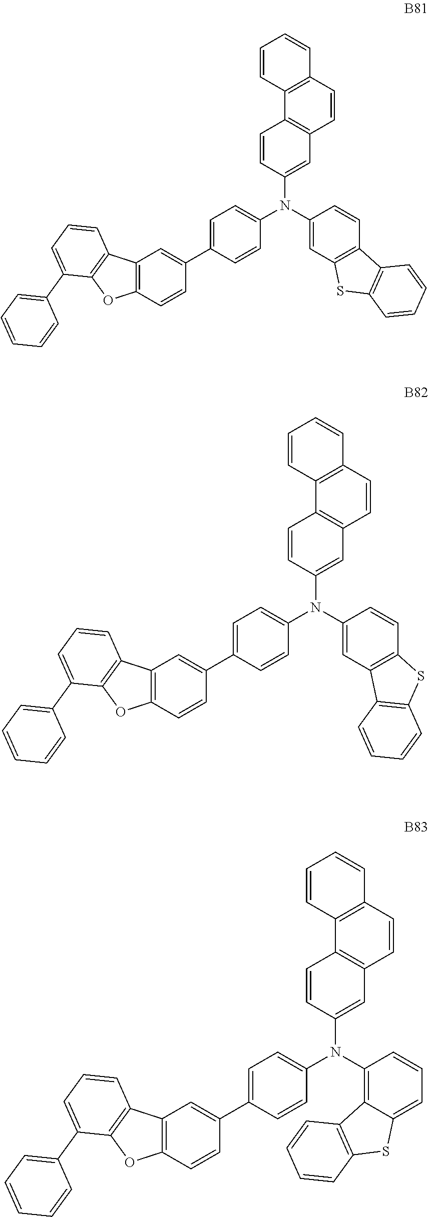 [SMILES: c1ccc(-c2cccc3c2oc2ccc(-c4ccc(N(c5ccc6c(ccc7ccccc76)c5)c5ccc6c(c5)sc5ccccc56)cc4)cc23)cc1 ; c1ccc(-c2cccc3c2oc2ccc(-c4ccc(N(c5ccc6c(ccc7ccccc76)c5)c5ccc6sc7ccccc7c6c5)cc4)cc23)cc1 ; c1ccc(-c2cccc3c2oc2ccc(-c4ccc(N(c5ccc6c(ccc7ccccc76)c5)c5cccc6sc7ccccc7c56)cc4)cc23)cc1]

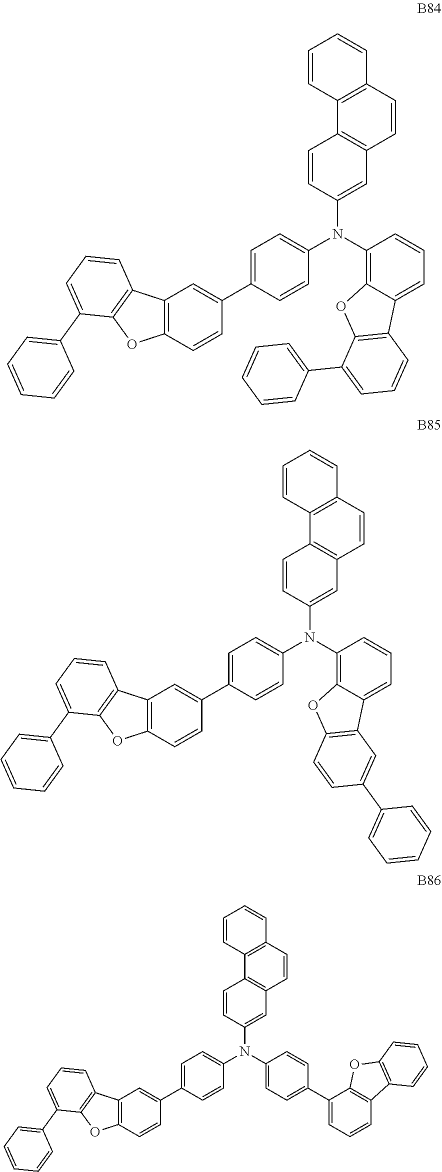 [SMILES: c1ccc(-c2cccc3c2oc2ccc(-c4ccc(N(c5ccc6c(ccc7ccccc76)c5)c5cccc6c5oc5c(-c7ccccc7)cccc56)cc4)cc23)cc1 ; c1ccc(-c2ccc3oc4c(N(c5ccc(-c6ccc7oc8c(-c9ccccc9)cccc8c7c6)cc5)c5ccc6c(ccc7ccccc76)c5)cccc4c3c2)cc1 ; c1ccc(-c2cccc3c2oc2ccc(-c4ccc(N(c5ccc(-c6cccc7c6oc6ccccc67)cc5)c5ccc6c(ccc7ccccc76)c5)cc4)cc23)cc1]

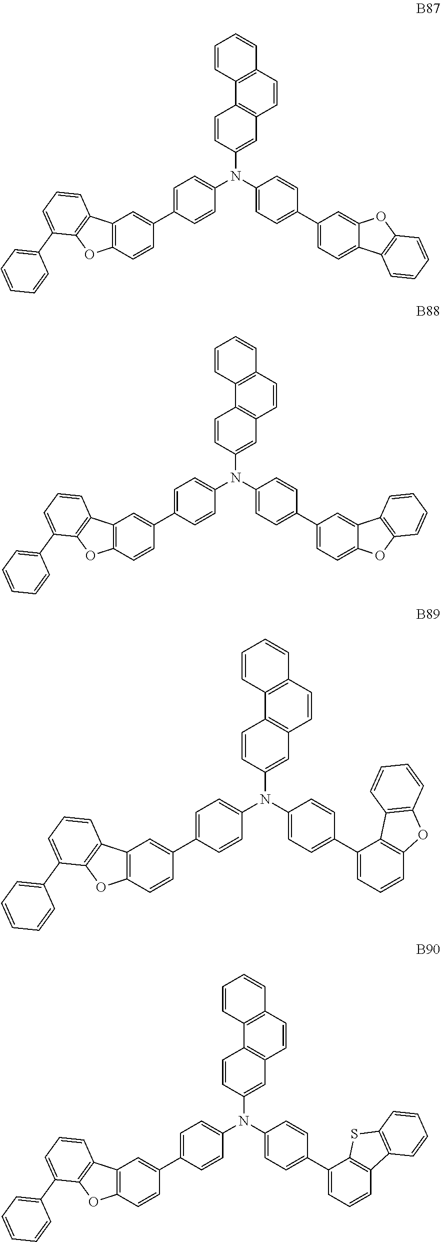 [SMILES: c1ccc(-c2cccc3c2oc2ccc(-c4ccc(N(c5ccc(-c6ccc7c(c6)oc6ccccc67)cc5)c5ccc6c(ccc7ccccc76)c5)cc4)cc23)cc1 ; c1ccc(-c2cccc3c2oc2ccc(-c4ccc(N(c5ccc(-c6ccc7oc8ccccc8c7c6)cc5)c5ccc6c(ccc7ccccc76)c5)cc4)cc23)cc1 ; c1ccc(-c2cccc3c2oc2ccc(-c4ccc(N(c5ccc(-c6cccc7oc8ccccc8c67)cc5)c5ccc6c(ccc7ccccc76)c5)cc4)cc23)cc1 ; c1ccc(-c2cccc3c2oc2ccc(-c4ccc(N(c5ccc(-c6cccc7c6sc6ccccc67)cc5)c5ccc6c(ccc7ccccc76)c5)cc4)cc23)cc1]

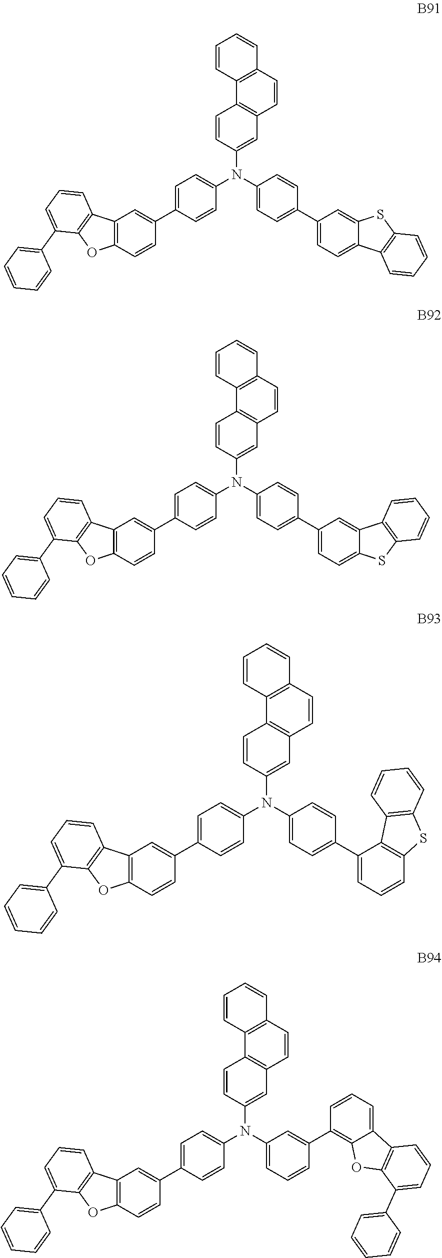 [SMILES: c1ccc(-c2cccc3c2oc2ccc(-c4ccc(N(c5ccc(-c6ccc7c(c6)sc6ccccc67)cc5)c5ccc6c(ccc7ccccc76)c5)cc4)cc23)cc1 ; c1ccc(-c2cccc3c2oc2ccc(-c4ccc(N(c5ccc(-c6ccc7sc8ccccc8c7c6)cc5)c5ccc6c(ccc7ccccc76)c5)cc4)cc23)cc1 ; c1ccc(-c2cccc3c2oc2ccc(-c4ccc(N(c5ccc(-c6cccc7sc8ccccc8c67)cc5)c5ccc6c(ccc7ccccc76)c5)cc4)cc23)cc1 ; c1ccc(-c2cccc3c2oc2ccc(-c4ccc(N(c5cccc(-c6cccc7c6oc6c(-c8ccccc8)cccc67)c5)c5ccc6c(ccc7ccccc76)c5)cc4)cc23)cc1]

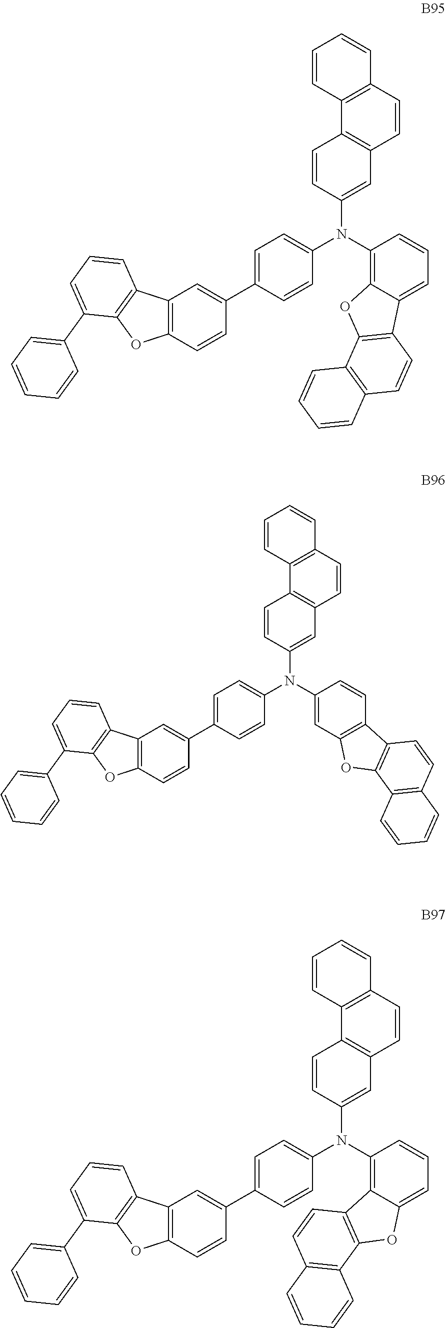 [SMILES: c1ccc(-c2cccc3c2oc2ccc(-c4ccc(N(c5ccc6c(ccc7ccccc76)c5)c5cccc6c5oc5c7ccccc7ccc65)cc4)cc23)cc1 ; c1ccc(-c2cccc3c2oc2ccc(-c4ccc(N(c5ccc6c(ccc7ccccc76)c5)c5ccc6c(c5)oc5c7ccccc7ccc65)cc4)cc23)cc1 ; c1ccc(-c2cccc3c2oc2ccc(-c4ccc(N(c5ccc6c(ccc7ccccc76)c5)c5cccc6oc7c8ccccc8ccc7c56)cc4)cc23)cc1]

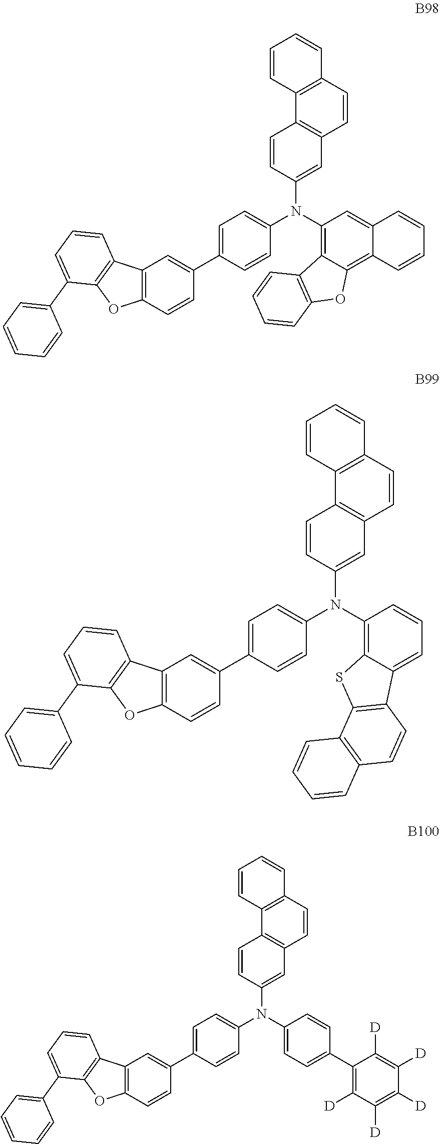 [SMILES: c1ccc(-c2cccc3c2oc2ccc(-c4ccc(N(c5ccc6c(ccc7ccccc76)c5)c5cc6ccccc6c6oc7ccccc7c56)cc4)cc23)cc1 ; c1ccc(-c2cccc3c2oc2ccc(-c4ccc(N(c5ccc6c(ccc7ccccc76)c5)c5cccc6c5sc5c7ccccc7ccc65)cc4)cc23)cc1 ; [2H]c1c([2H])c([2H])c(-c2ccc(N(c3ccc(-c4ccc5oc6c(-c7ccccc7)cccc6c5c4)cc3)c3ccc4c(ccc5ccccc54)c3)cc2)c([2H])c1[2H]]

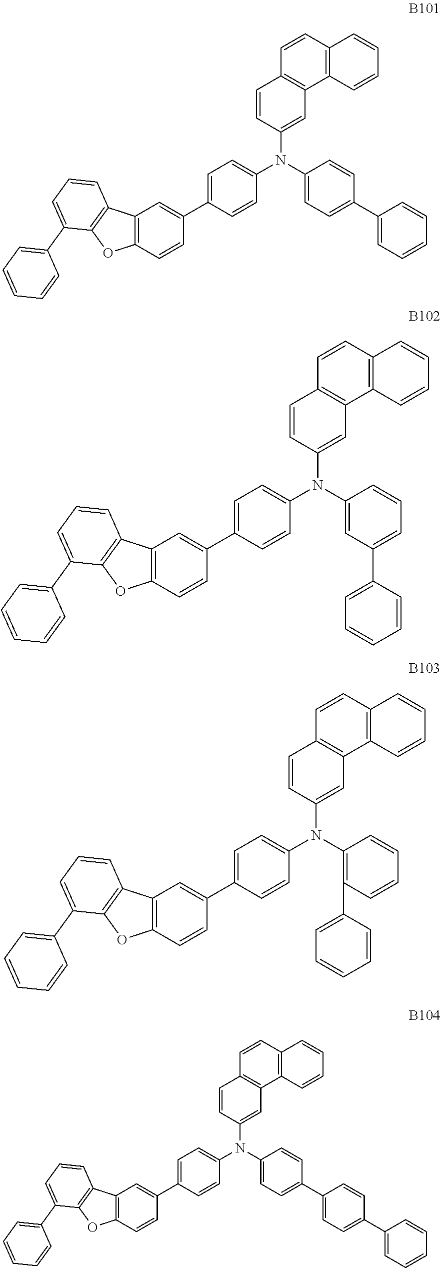 [SMILES: c1ccc(-c2ccc(N(c3ccc(-c4ccc5oc6c(-c7ccccc7)cccc6c5c4)cc3)c3ccc4ccc5ccccc5c4c3)cc2)cc1 ; c1ccc(-c2cccc(N(c3ccc(-c4ccc5oc6c(-c7ccccc7)cccc6c5c4)cc3)c3ccc4ccc5ccccc5c4c3)c2)cc1 ; c1ccc(-c2ccccc2N(c2ccc(-c3ccc4oc5c(-c6ccccc6)cccc5c4c3)cc2)c2ccc3ccc4ccccc4c3c2)cc1 ; c1ccc(-c2ccc(-c3ccc(N(c4ccc(-c5ccc6oc7c(-c8ccccc8)cccc7c6c5)cc4)c4ccc5ccc6ccccc6c5c4)cc3)cc2)cc1]

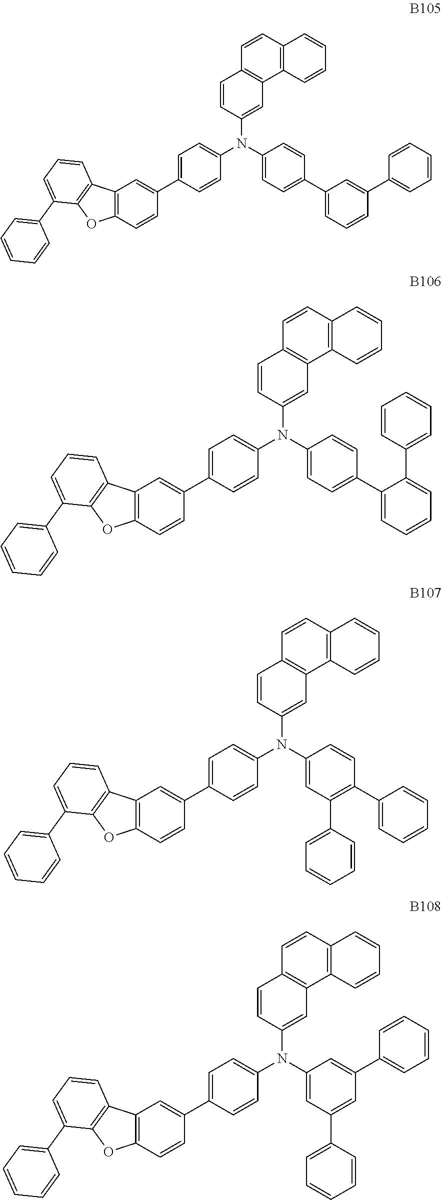 [SMILES: c1ccc(-c2cccc(-c3ccc(N(c4ccc(-c5ccc6oc7c(-c8ccccc8)cccc7c6c5)cc4)c4ccc5ccc6ccccc6c5c4)cc3)c2)cc1 ; c1ccc(-c2ccccc2-c2ccc(N(c3ccc(-c4ccc5oc6c(-c7ccccc7)cccc6c5c4)cc3)c3ccc4ccc5ccccc5c4c3)cc2)cc1 ; c1ccc(-c2ccc(N(c3ccc(-c4ccc5oc6c(-c7ccccc7)cccc6c5c4)cc3)c3ccc4ccc5ccccc5c4c3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc4ccc5ccccc5c4c3)c2)cc1]

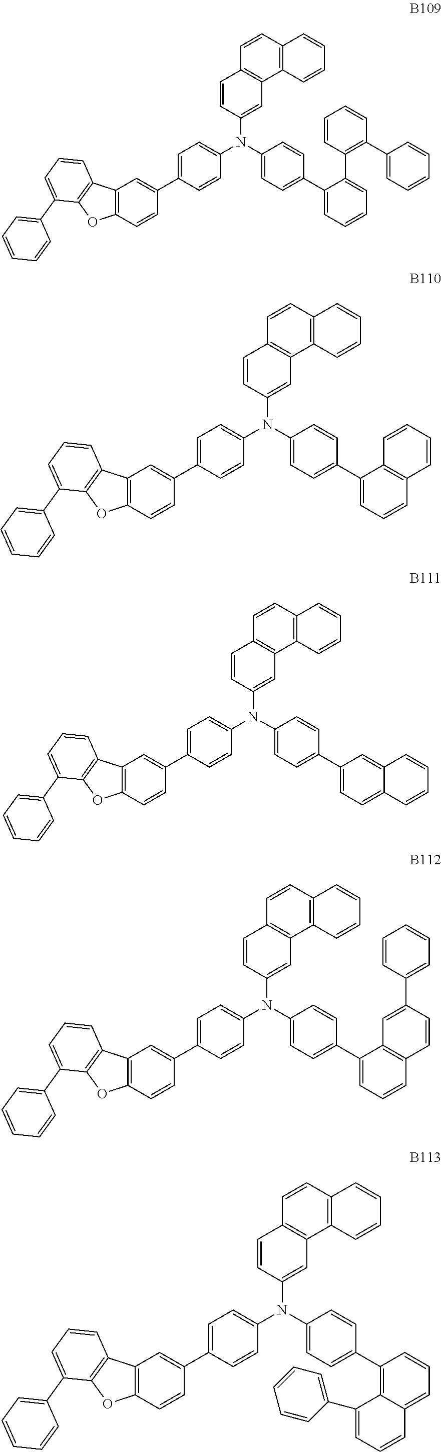

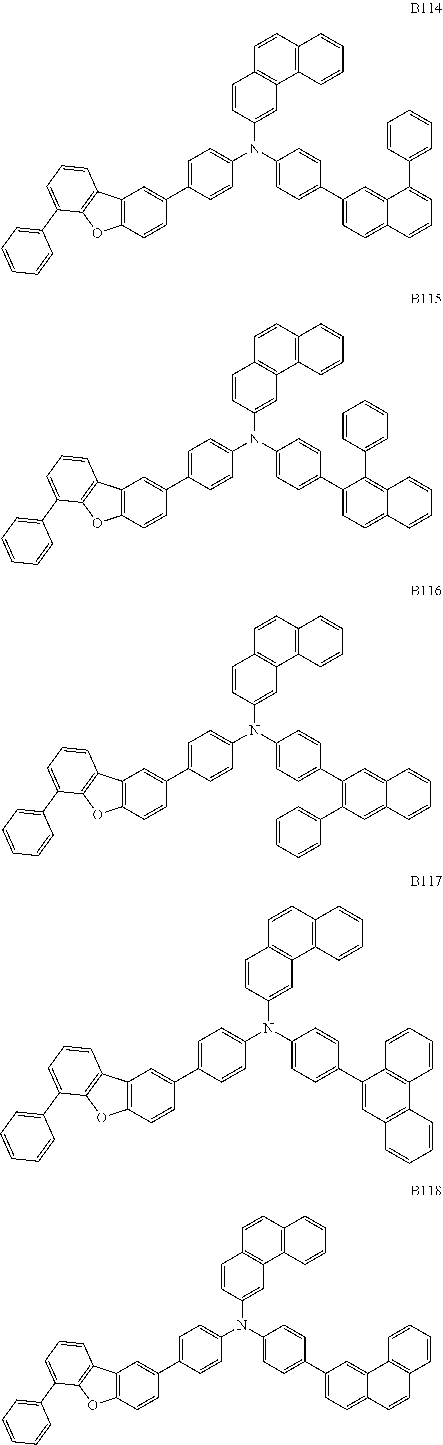

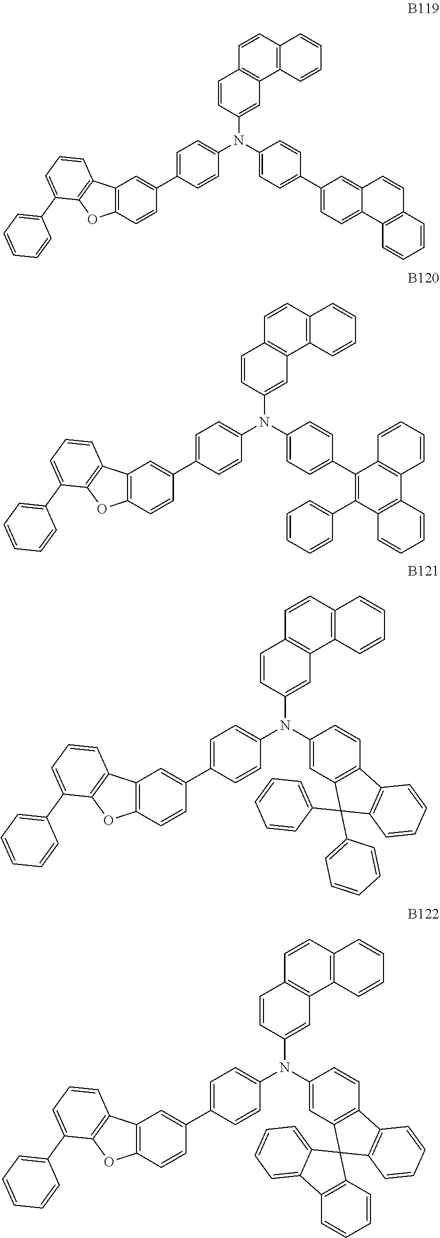 [SMILES: c1ccc(-c2cccc3c2oc2ccc(-c4ccc(N(c5ccc(-c6ccc7c(ccc8ccccc87)c6)cc5)c5ccc6ccc7ccccc7c6c5)cc4)cc23)cc1 ; c1ccc(-c2c(-c3ccc(N(c4ccc(-c5ccc6oc7c(-c8ccccc8)cccc7c6c5)cc4)c4ccc5ccc6ccccc6c5c4)cc3)c3ccccc3c3ccccc23)cc1 ; c1ccc(-c2cccc3c2oc2ccc(-c4ccc(N(c5ccc6c(c5)C(c5ccccc5)(c5ccccc5)c5ccccc5-6)c5ccc6ccc7ccccc7c6c5)cc4)cc23)cc1 ; c1ccc(-c2cccc3c2oc2ccc(-c4ccc(N(c5ccc6c(c5)C5(c7ccccc7-c7ccccc75)c5ccccc5-6)c5ccc6ccc7ccccc7c6c5)cc4)cc23)cc1]

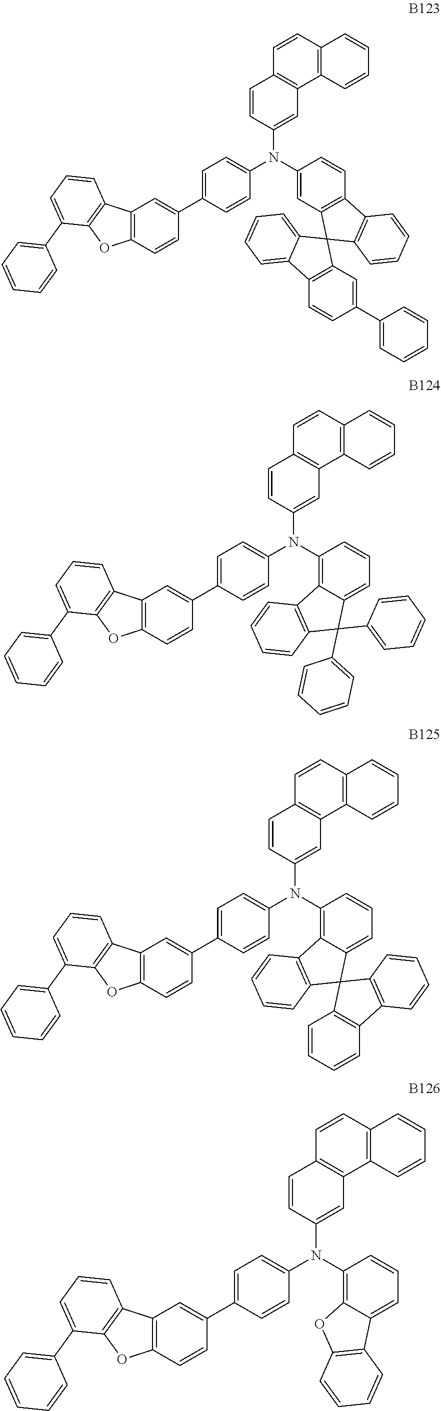 [SMILES: c1ccc(-c2ccc3c(c2)C2(c4ccccc4-3)c3ccccc3-c3ccc(N(c4ccc(-c5ccc6oc7c(-c8ccccc8)cccc7c6c5)cc4)c4ccc5ccc6ccccc6c5c4)cc32)cc1 ; c1ccc(-c2cccc3c2oc2ccc(-c4ccc(N(c5ccc6ccc7ccccc7c6c5)c5cccc6c5-c5ccccc5C6(c5ccccc5)c5ccccc5)cc4)cc23)cc1 ; c1ccc(-c2cccc3c2oc2ccc(-c4ccc(N(c5ccc6ccc7ccccc7c6c5)c5cccc6c5-c5ccccc5C65c6ccccc6-c6ccccc65)cc4)cc23)cc1 ; c1ccc(-c2cccc3c2oc2ccc(-c4ccc(N(c5ccc6ccc7ccccc7c6c5)c5cccc6c5oc5ccccc56)cc4)cc23)cc1]

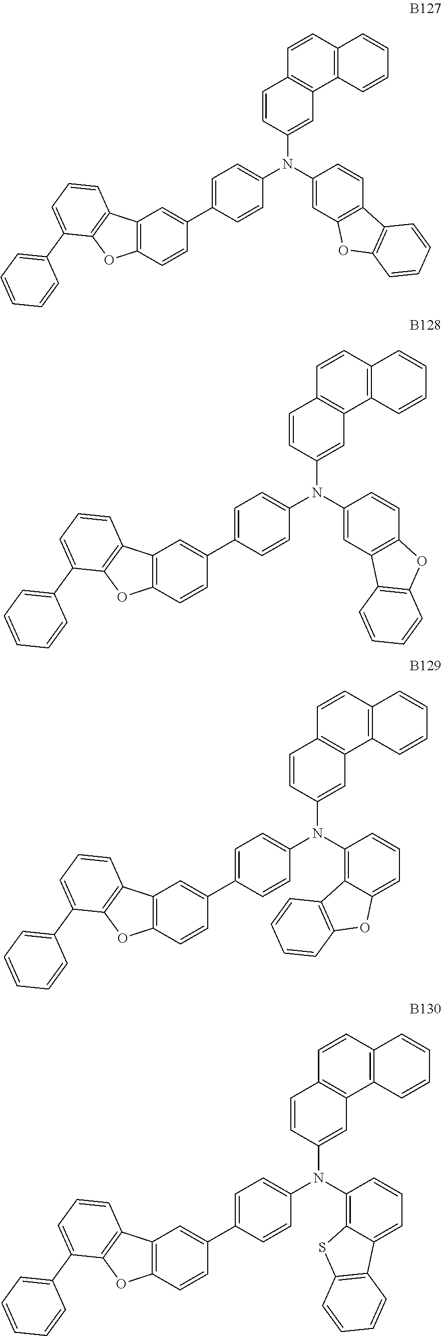 [SMILES: c1ccc(-c2cccc3c2oc2ccc(-c4ccc(N(c5ccc6c(c5)oc5ccccc56)c5ccc6ccc7ccccc7c6c5)cc4)cc23)cc1 ; c1ccc(-c2cccc3c2oc2ccc(-c4ccc(N(c5ccc6ccc7ccccc7c6c5)c5ccc6oc7ccccc7c6c5)cc4)cc23)cc1 ; c1ccc(-c2cccc3c2oc2ccc(-c4ccc(N(c5ccc6ccc7ccccc7c6c5)c5cccc6oc7ccccc7c56)cc4)cc23)cc1 ; c1ccc(-c2cccc3c2oc2ccc(-c4ccc(N(c5ccc6ccc7ccccc7c6c5)c5cccc6c5sc5ccccc56)cc4)cc23)cc1]

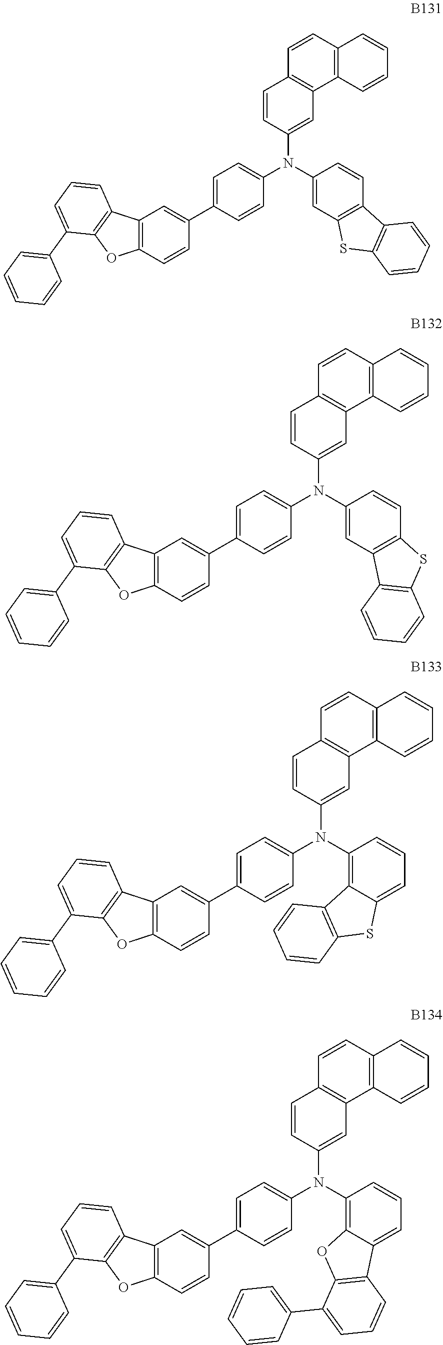 [SMILES: c1ccc(-c2cccc3c2oc2ccc(-c4ccc(N(c5ccc6c(c5)sc5ccccc56)c5ccc6ccc7ccccc7c6c5)cc4)cc23)cc1 ; c1ccc(-c2cccc3c2oc2ccc(-c4ccc(N(c5ccc6ccc7ccccc7c6c5)c5ccc6sc7ccccc7c6c5)cc4)cc23)cc1 ; c1ccc(-c2cccc3c2oc2ccc(-c4ccc(N(c5ccc6ccc7ccccc7c6c5)c5cccc6sc7ccccc7c56)cc4)cc23)cc1 ; c1ccc(-c2cccc3c2oc2ccc(-c4ccc(N(c5ccc6ccc7ccccc7c6c5)c5cccc6c5oc5c(-c7ccccc7)cccc56)cc4)cc23)cc1]

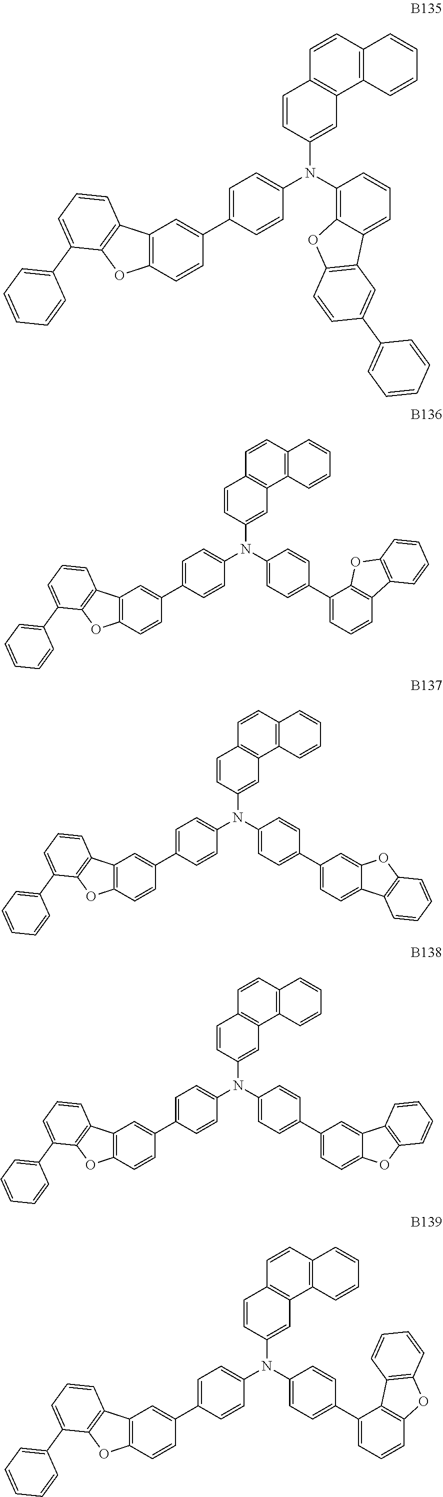 [SMILES: c1ccc(-c2ccc3oc4c(N(c5ccc(-c6ccc7oc8c(-c9ccccc9)cccc8c7c6)cc5)c5ccc6ccc7ccccc7c6c5)cccc4c3c2)cc1 ; c1ccc(-c2cccc3c2oc2ccc(-c4ccc(N(c5ccc(-c6cccc7c6oc6ccccc67)cc5)c5ccc6ccc7ccccc7c6c5)cc4)cc23)cc1 ; c1ccc(-c2cccc3c2oc2ccc(-c4ccc(N(c5ccc(-c6ccc7c(c6)oc6ccccc67)cc5)c5ccc6ccc7ccccc7c6c5)cc4)cc23)cc1 ; c1ccc(-c2cccc3c2oc2ccc(-c4ccc(N(c5ccc(-c6ccc7oc8ccccc8c7c6)cc5)c5ccc6ccc7ccccc7c6c5)cc4)cc23)cc1 ; c1ccc(-c2cccc3c2oc2ccc(-c4ccc(N(c5ccc(-c6cccc7oc8ccccc8c67)cc5)c5ccc6ccc7ccccc7c6c5)cc4)cc23)cc1]

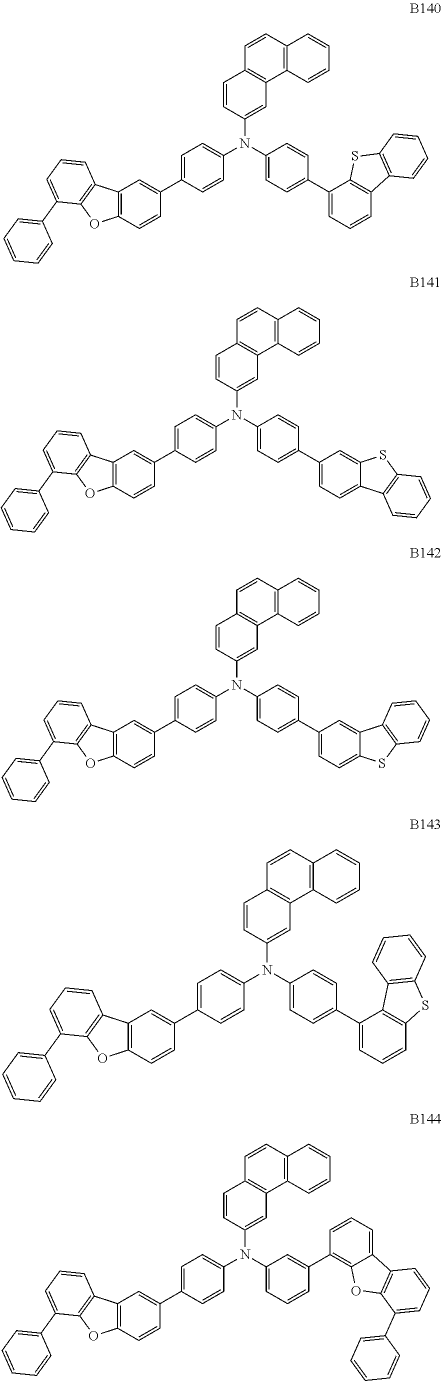

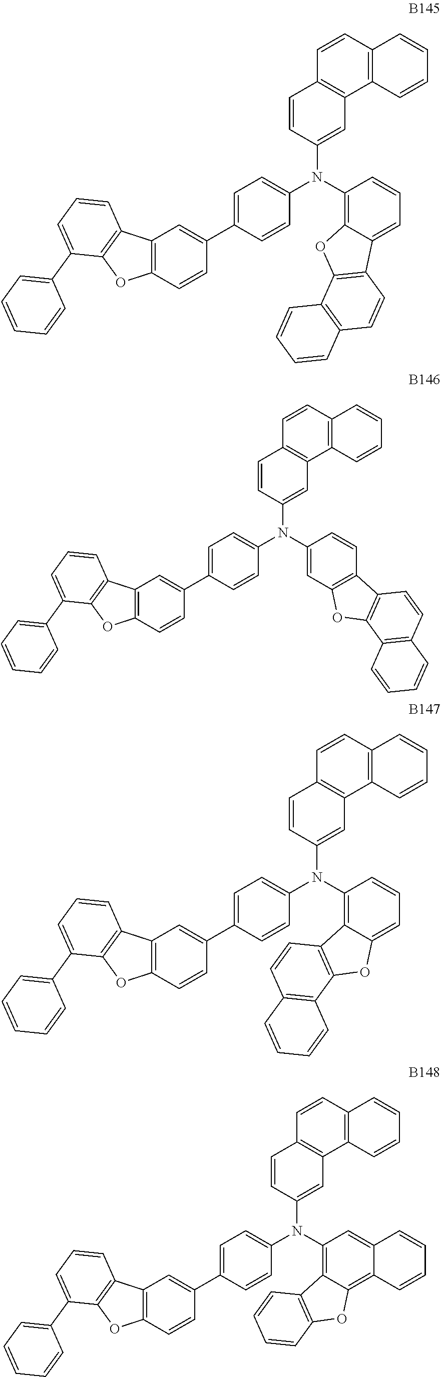 [SMILES: c1ccc(-c2cccc3c2oc2ccc(-c4ccc(N(c5ccc6ccc7ccccc7c6c5)c5cccc6c5oc5c7ccccc7ccc65)cc4)cc23)cc1 ; c1ccc(-c2cccc3c2oc2ccc(-c4ccc(N(c5ccc6c(c5)oc5c7ccccc7ccc65)c5ccc6ccc7ccccc7c6c5)cc4)cc23)cc1 ; c1ccc(-c2cccc3c2oc2ccc(-c4ccc(N(c5ccc6ccc7ccccc7c6c5)c5cccc6oc7c8ccccc8ccc7c56)cc4)cc23)cc1 ; c1ccc(-c2cccc3c2oc2ccc(-c4ccc(N(c5ccc6ccc7ccccc7c6c5)c5cc6ccccc6c6oc7ccccc7c56)cc4)cc23)cc1]

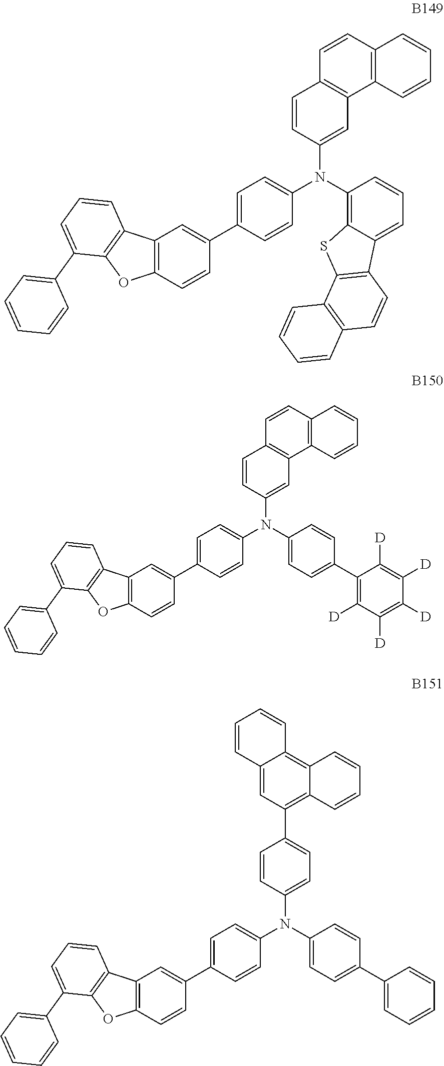 [SMILES: c1ccc(-c2cccc3c2oc2ccc(-c4ccc(N(c5ccc6ccc7ccccc7c6c5)c5cccc6c5sc5c7ccccc7ccc65)cc4)cc23)cc1 ; [2H]c1c([2H])c([2H])c(-c2ccc(N(c3ccc(-c4ccc5oc6c(-c7ccccc7)cccc6c5c4)cc3)c3ccc4ccc5ccccc5c4c3)cc2)c([2H])c1[2H] ; c1ccc(-c2ccc(N(c3ccc(-c4ccc5oc6c(-c7ccccc7)cccc6c5c4)cc3)c3ccc(-c4cc5ccccc5c5ccccc45)cc3)cc2)cc1]

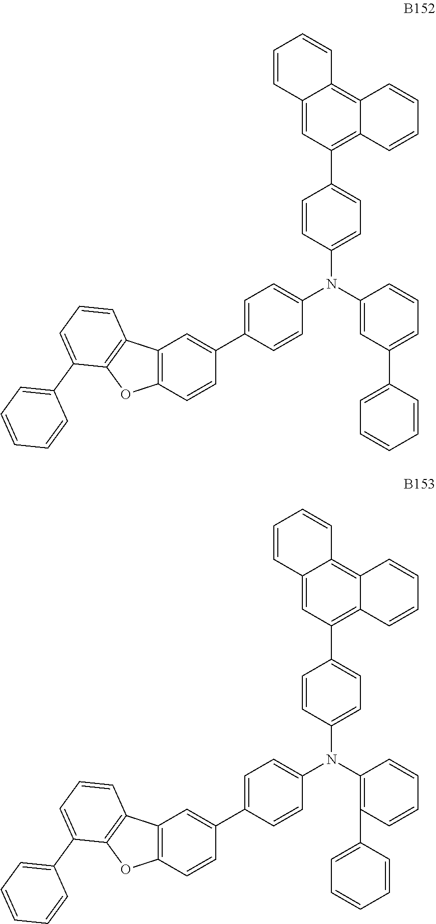 [SMILES: c1ccc(-c2cccc(N(c3ccc(-c4ccc5oc6c(-c7ccccc7)cccc6c5c4)cc3)c3ccc(-c4cc5ccccc5c5ccccc45)cc3)c2)cc1 ; c1ccc(-c2ccccc2N(c2ccc(-c3ccc4oc5c(-c6ccccc6)cccc5c4c3)cc2)c2ccc(-c3cc4ccccc4c4ccccc34)cc2)cc1]

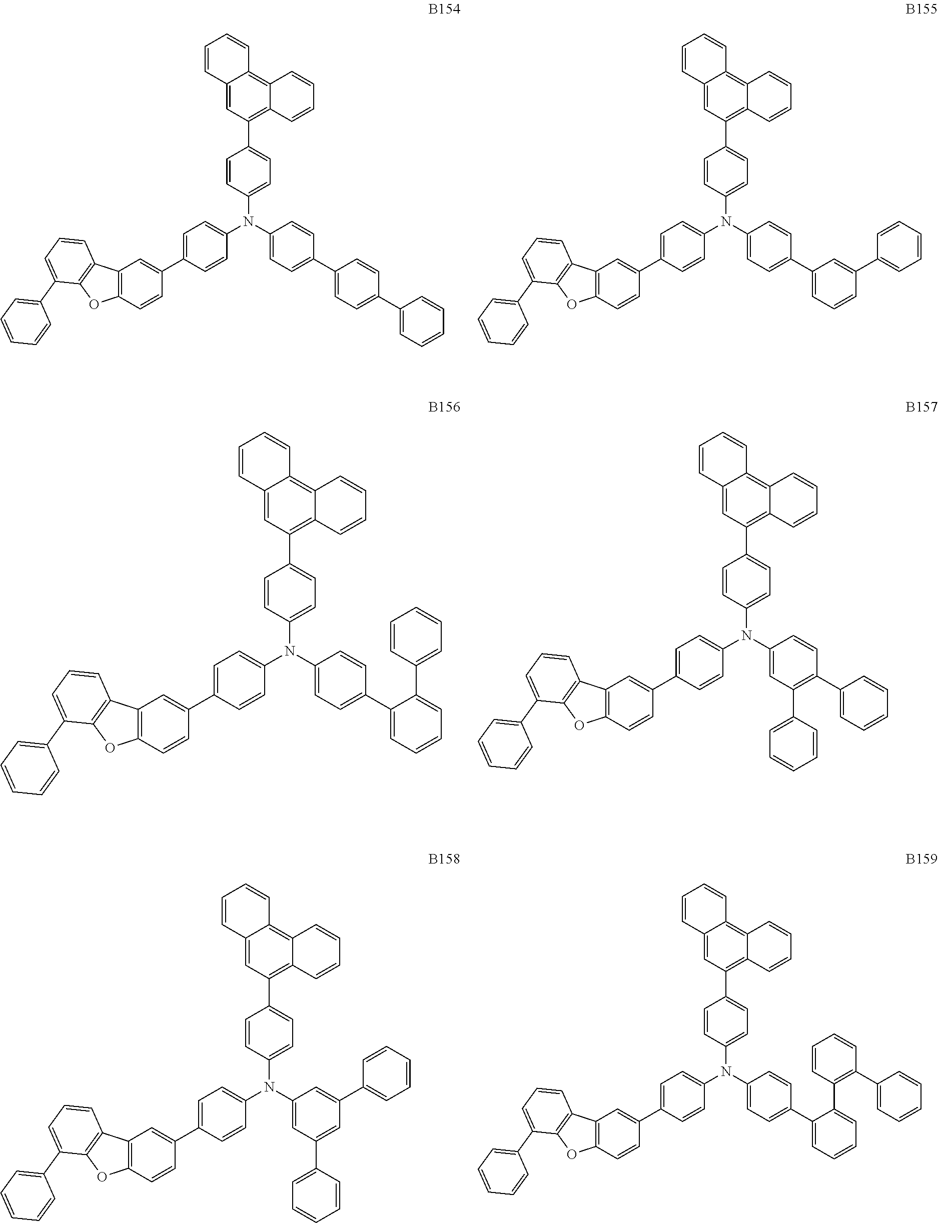

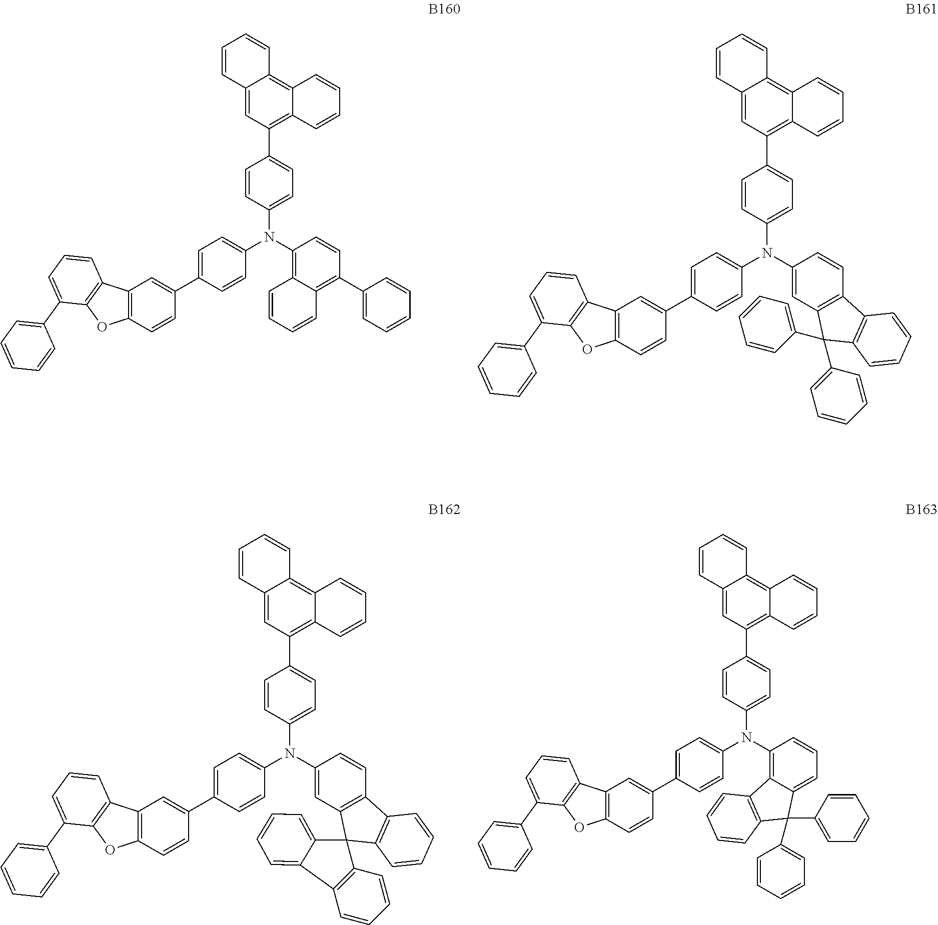 [SMILES: c1ccc(-c2ccc(N(c3ccc(-c4ccc5oc6c(-c7ccccc7)cccc6c5c4)cc3)c3ccc(-c4cc5ccccc5c5ccccc45)cc3)c3ccccc23)cc1 ; c1ccc(-c2cccc3c2oc2ccc(-c4ccc(N(c5ccc(-c6cc7ccccc7c7ccccc67)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6cc7ccccc7c7ccccc67)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6cc7ccccc7c7ccccc67)cc5)c5cccc6c5-c5ccccc5C6(c5ccccc5)c5ccccc5)cc4)cc23)cc1]

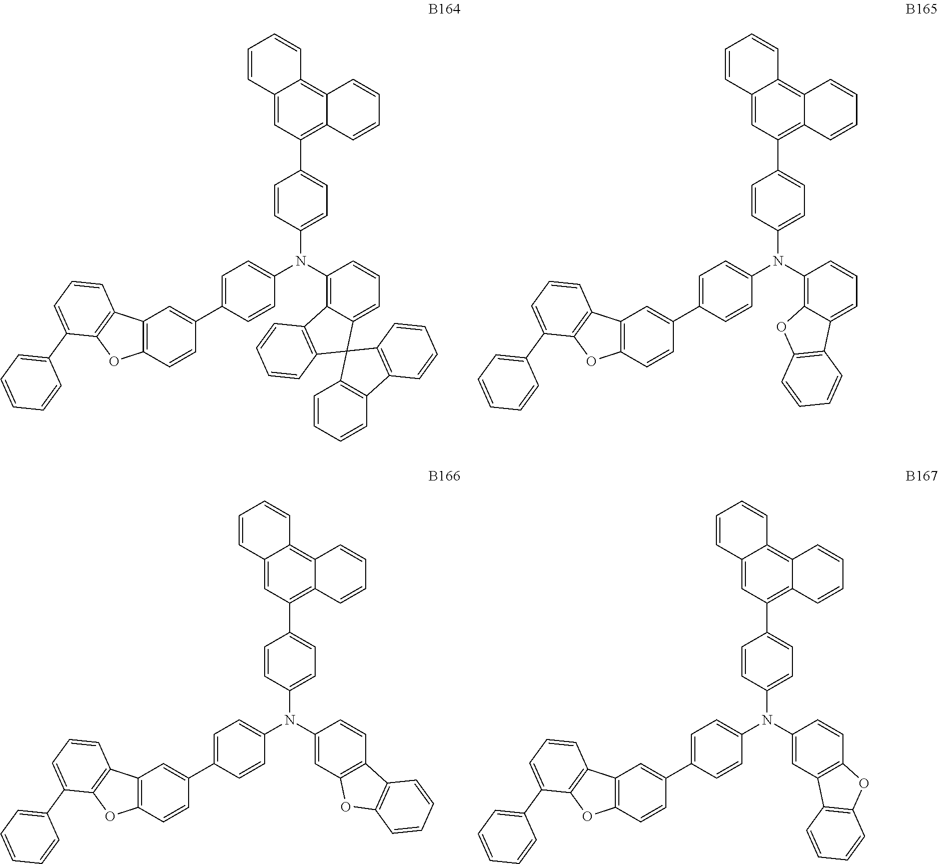 [SMILES: c1ccc(-c2cccc3c2oc2ccc(-c4ccc(N(c5ccc(-c6cc7ccccc7c7ccccc67)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6cc7ccccc7c7ccccc67)cc5)c5cccc6c5oc5ccccc56)cc4)cc23)cc1 ; c1ccc(-c2cccc3c2oc2ccc(-c4ccc(N(c5ccc(-c6cc7ccccc7c7ccccc67)cc5)c5ccc6c(c5)oc5ccccc56)cc4)cc23)cc1 ; c1ccc(-c2cccc3c2oc2ccc(-c4ccc(N(c5ccc(-c6cc7ccccc7c7ccccc67)cc5)c5ccc6oc7ccccc7c6c5)cc4)cc23)cc1]

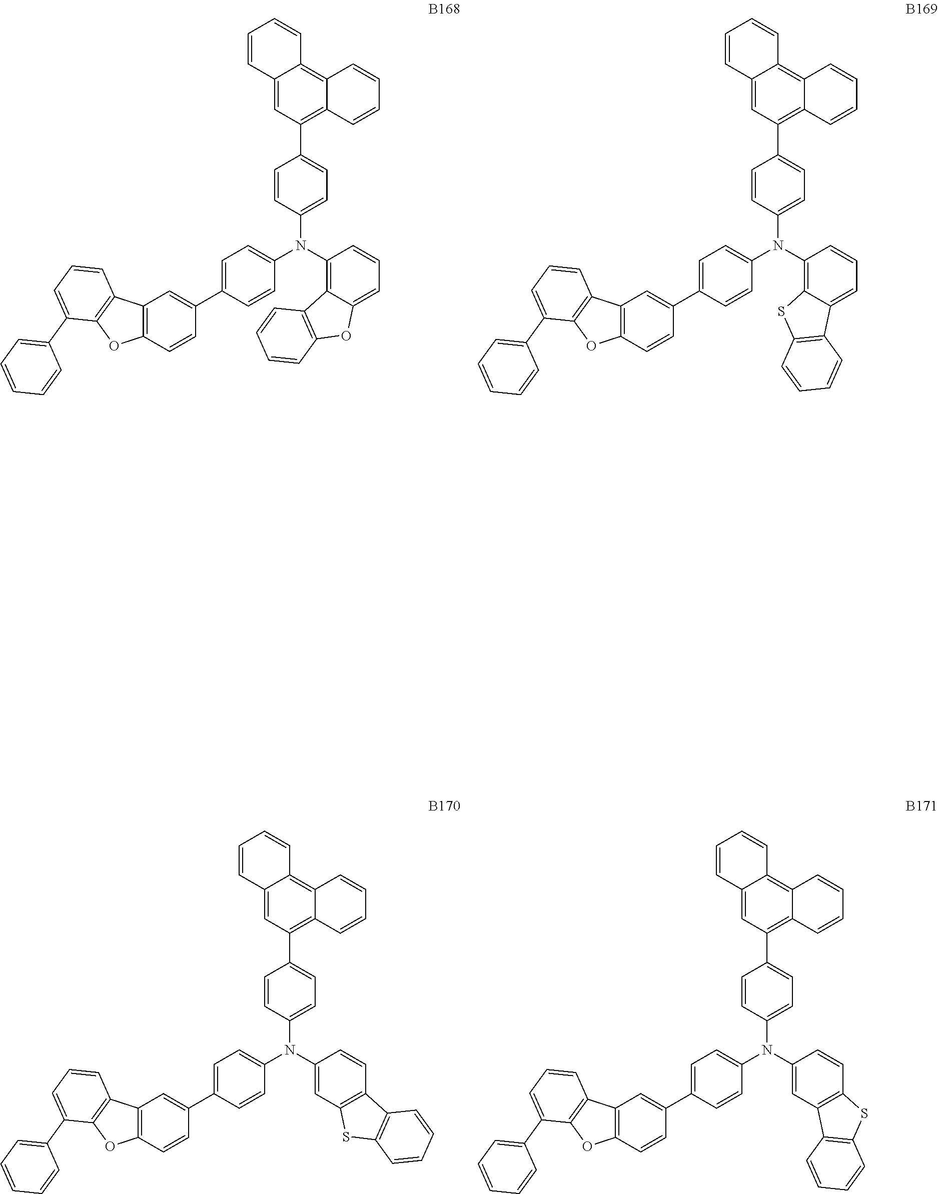 [SMILES: c1ccc(-c2cccc3c2oc2ccc(-c4ccc(N(c5ccc(-c6cc7ccccc7c7ccccc67)cc5)c5cccc6oc7ccccc7c56)cc4)cc23)cc1 ; c1ccc(-c2cccc3c2oc2ccc(-c4ccc(N(c5ccc(-c6cc7ccccc7c7ccccc67)cc5)c5cccc6c5sc5ccccc56)cc4)cc23)cc1 ; c1ccc(-c2cccc3c2oc2ccc(-c4ccc(N(c5ccc(-c6cc7ccccc7c7ccccc67)cc5)c5ccc6c(c5)sc5ccccc56)cc4)cc23)cc1 ; c1ccc(-c2cccc3c2oc2ccc(-c4ccc(N(c5ccc(-c6cc7ccccc7c7ccccc67)cc5)c5ccc6sc7ccccc7c6c5)cc4)cc23)cc1]

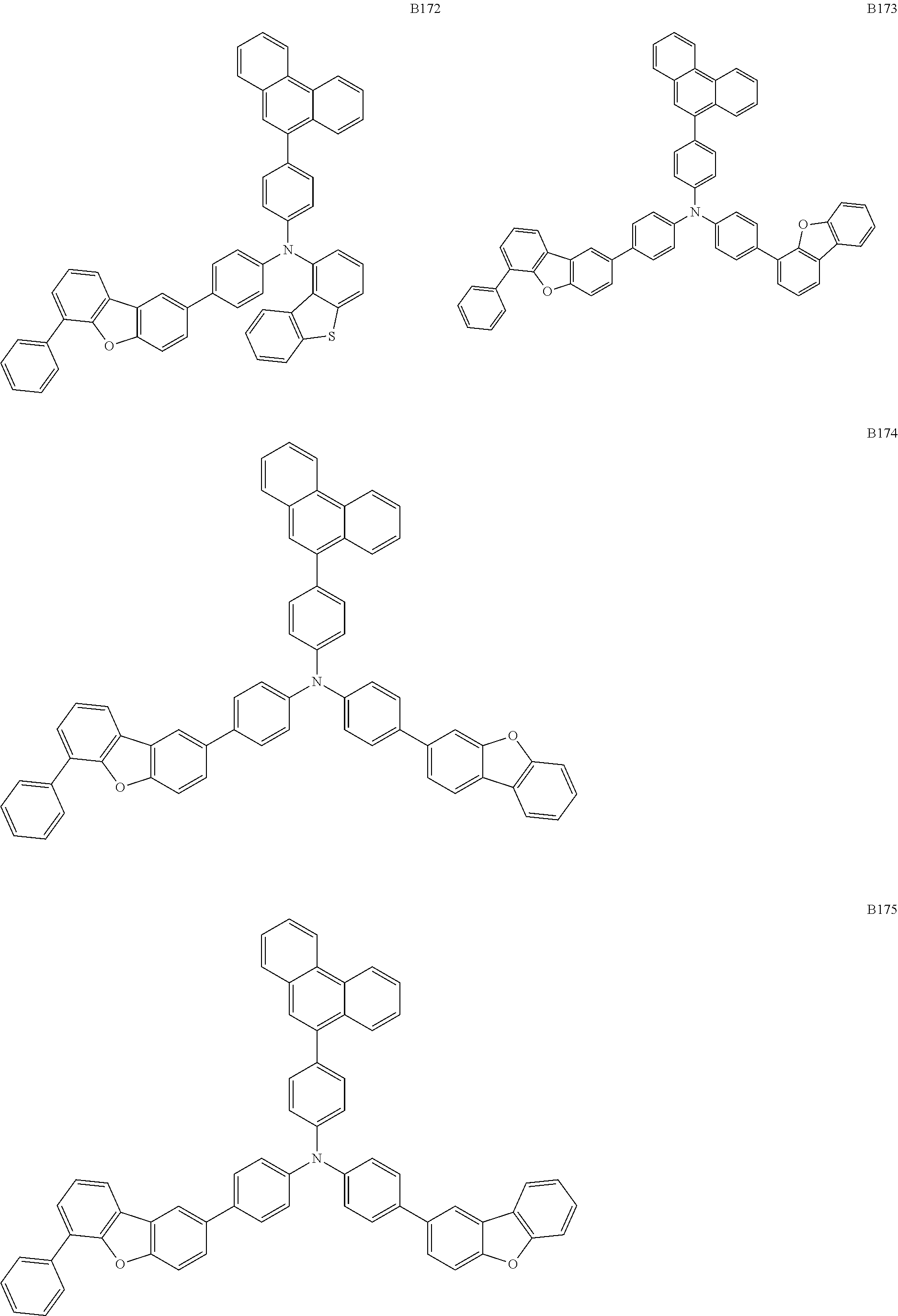 [SMILES: c1ccc(-c2cccc3c2oc2ccc(-c4ccc(N(c5ccc(-c6cc7ccccc7c7ccccc67)cc5)c5cccc6sc7ccccc7c56)cc4)cc23)cc1 ; c1ccc(-c2cccc3c2oc2ccc(-c4ccc(N(c5ccc(-c6cc7ccccc7c7ccccc67)cc5)c5ccc(-c6cccc7c6oc6ccccc67)cc5)cc4)cc23)cc1 ; c1ccc(-c2cccc3c2oc2ccc(-c4ccc(N(c5ccc(-c6ccc7c(c6)oc6ccccc67)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7oc8ccccc8c7c6)cc5)c5ccc(-c6cc7ccccc7c7ccccc67)cc5)cc4)cc23)cc1]

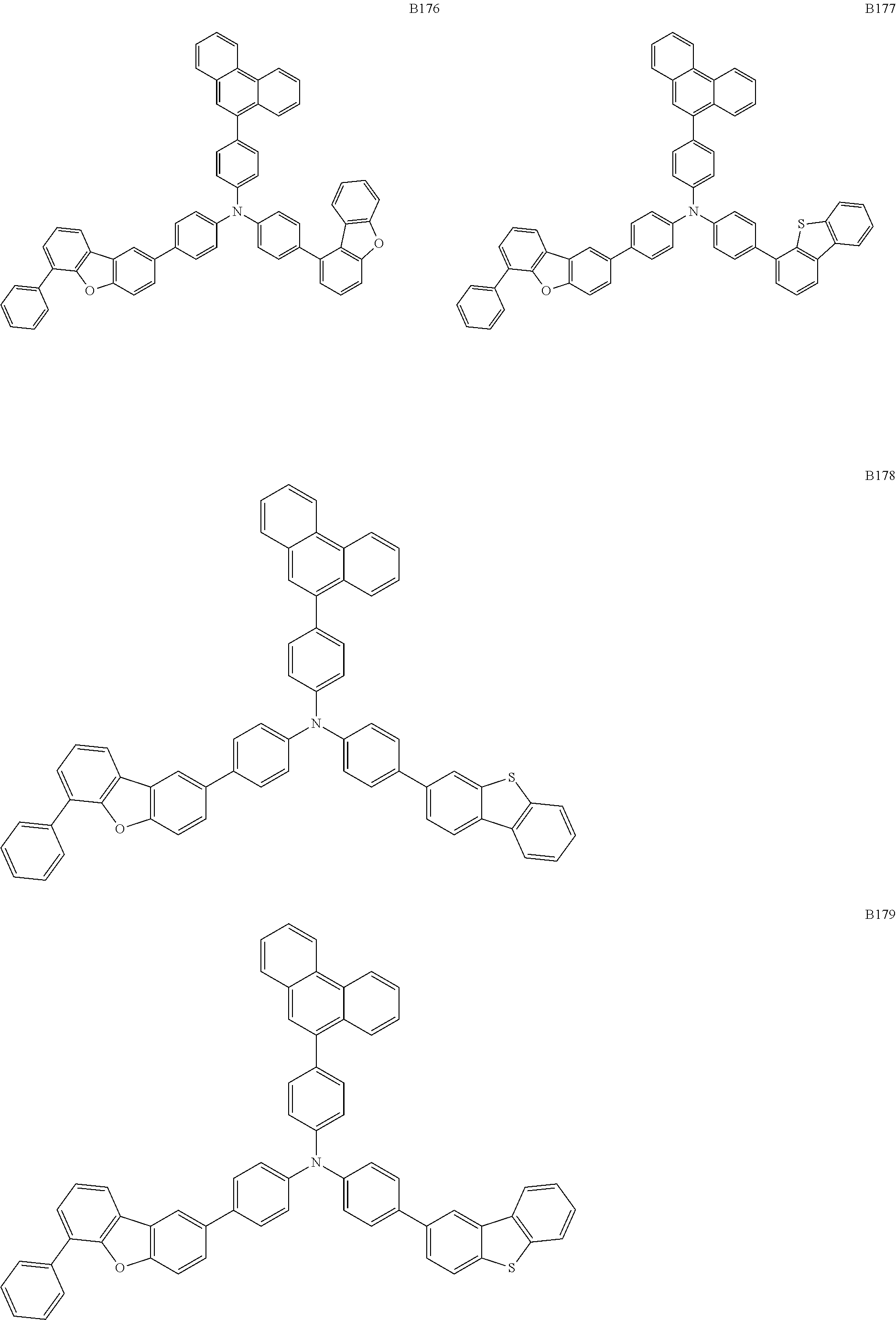 [SMILES: c1ccc(-c2cccc3c2oc2ccc(-c4ccc(N(c5ccc(-c6cc7ccccc7c7ccccc67)cc5)c5ccc(-c6cccc7oc8ccccc8c67)cc5)cc4)cc23)cc1 ; c1ccc(-c2cccc3c2oc2ccc(-c4ccc(N(c5ccc(-c6cc7ccccc7c7ccccc67)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7sc8ccccc8c7c6)cc5)c5ccc(-c6cc7ccccc7c7ccccc67)cc5)cc4)cc23)cc1]

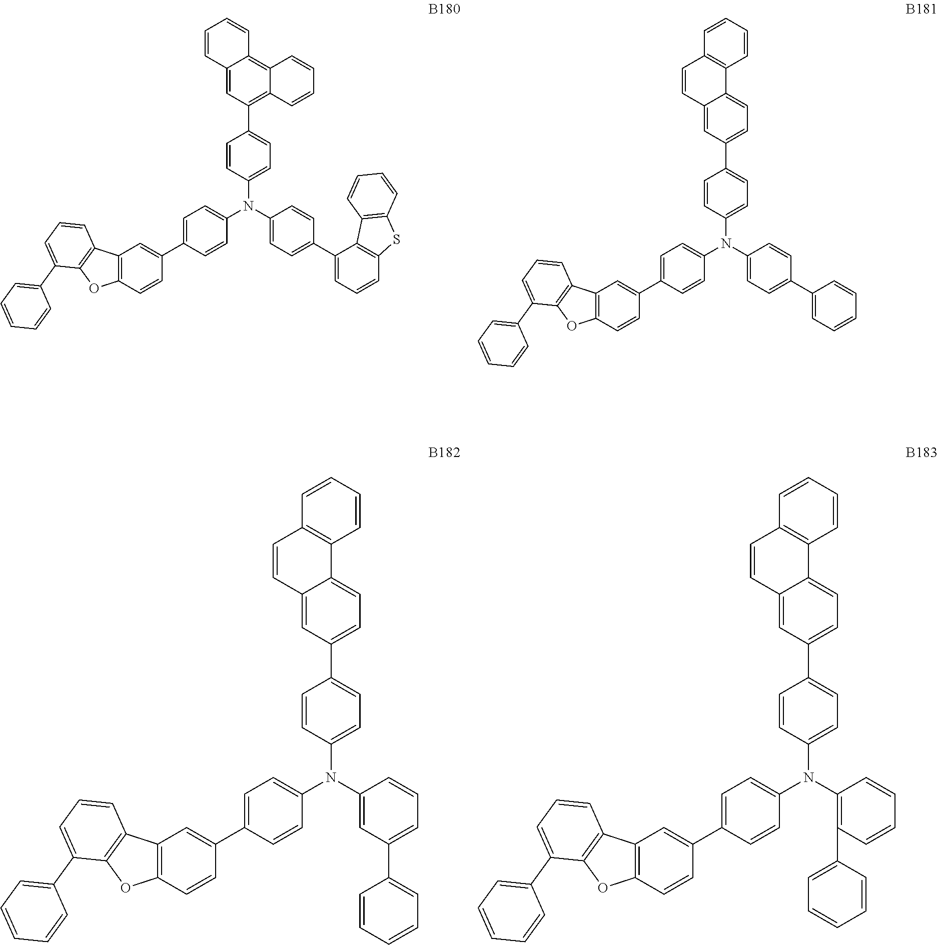 [SMILES: c1ccc(-c2cccc3c2oc2ccc(-c4ccc(N(c5ccc(-c6cc7ccccc7c7ccccc67)cc5)c5ccc(-c6cccc7sc8ccccc8c67)cc5)cc4)cc23)cc1 ; c1ccc(-c2ccc(N(c3ccc(-c4ccc5c(ccc6ccccc65)c4)cc3)c3ccc(-c4ccc5oc6c(-c7ccccc7)cccc6c5c4)cc3)cc2)cc1 ; c1ccc(-c2cccc(N(c3ccc(-c4ccc5c(ccc6ccccc65)c4)cc3)c3ccc(-c4ccc5oc6c(-c7ccccc7)cccc6c5c4)cc3)c2)cc1 ; c1ccc(-c2ccccc2N(c2ccc(-c3ccc4c(ccc5ccccc54)c3)cc2)c2ccc(-c3ccc4oc5c(-c6ccccc6)cccc5c4c3)cc2)cc1]

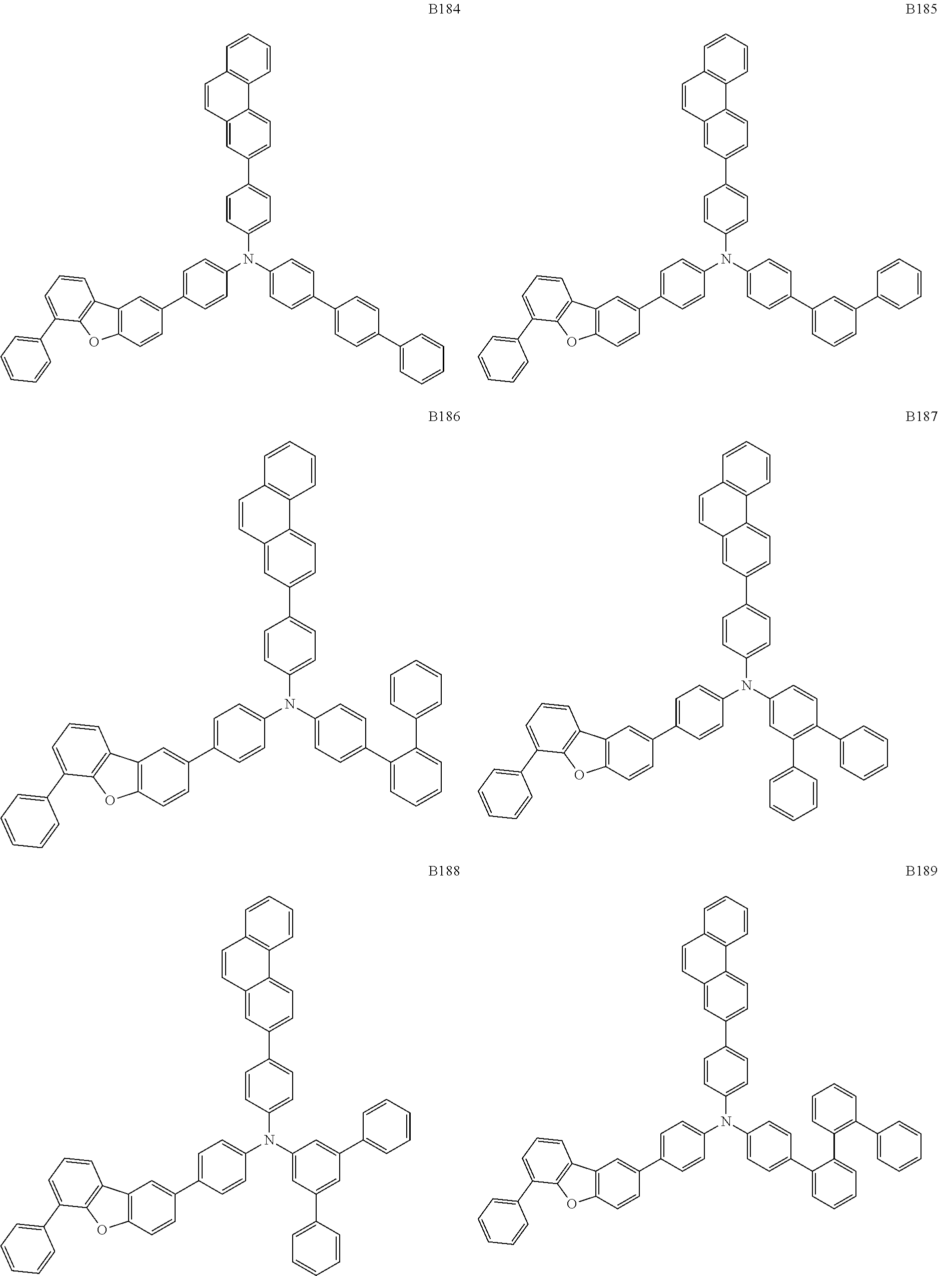

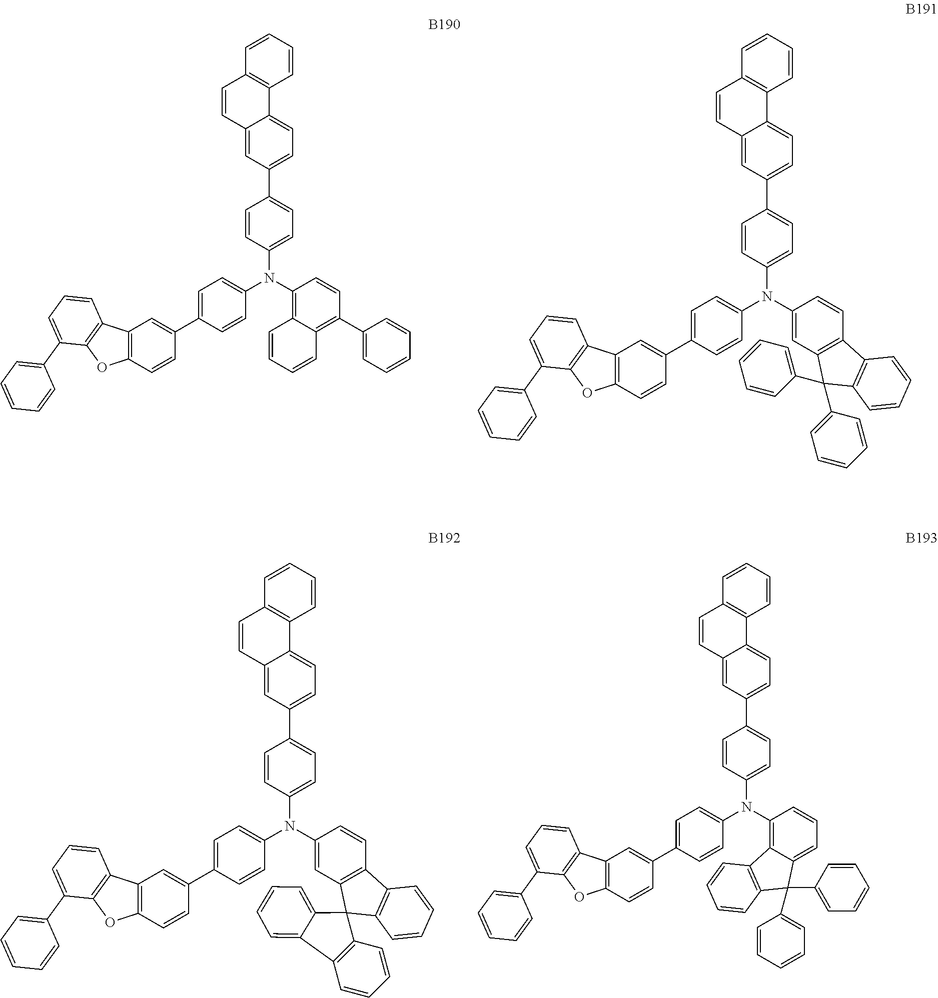 [SMILES: c1ccc(-c2ccc(N(c3ccc(-c4ccc5c(ccc6ccccc65)c4)cc3)c3ccc(-c4ccc5oc6c(-c7ccccc7)cccc6c5c4)cc3)c3ccccc23)cc1 ; c1ccc(-c2cccc3c2oc2ccc(-c4ccc(N(c5ccc(-c6ccc7c(ccc8ccccc87)c6)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6ccc7c(ccc8ccccc87)c6)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6ccc7c(ccc8ccccc87)c6)cc5)c5cccc6c5-c5ccccc5C6(c5ccccc5)c5ccccc5)cc4)cc23)cc1]

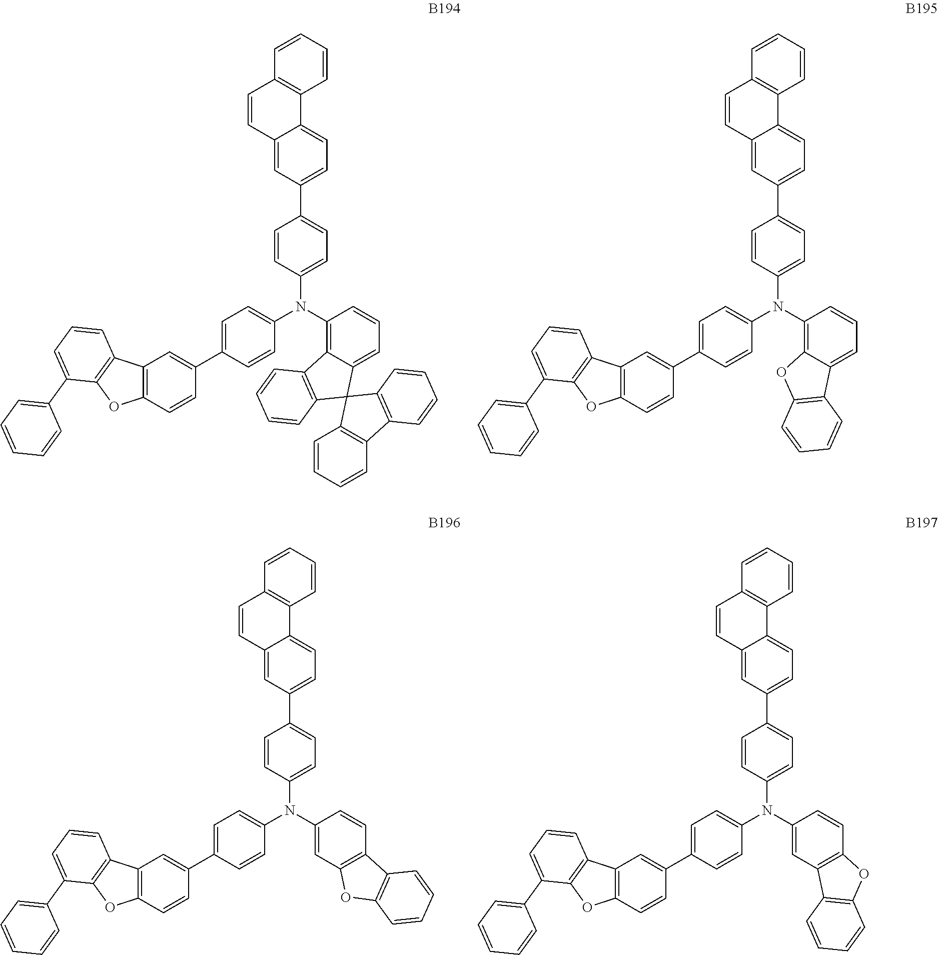 [SMILES: c1ccc(-c2cccc3c2oc2ccc(-c4ccc(N(c5ccc(-c6ccc7c(ccc8ccccc87)c6)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6ccc7c(ccc8ccccc87)c6)cc5)c5cccc6c5oc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6c(c5)oc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6oc7ccccc7c6c5)cc4)cc23)cc1]

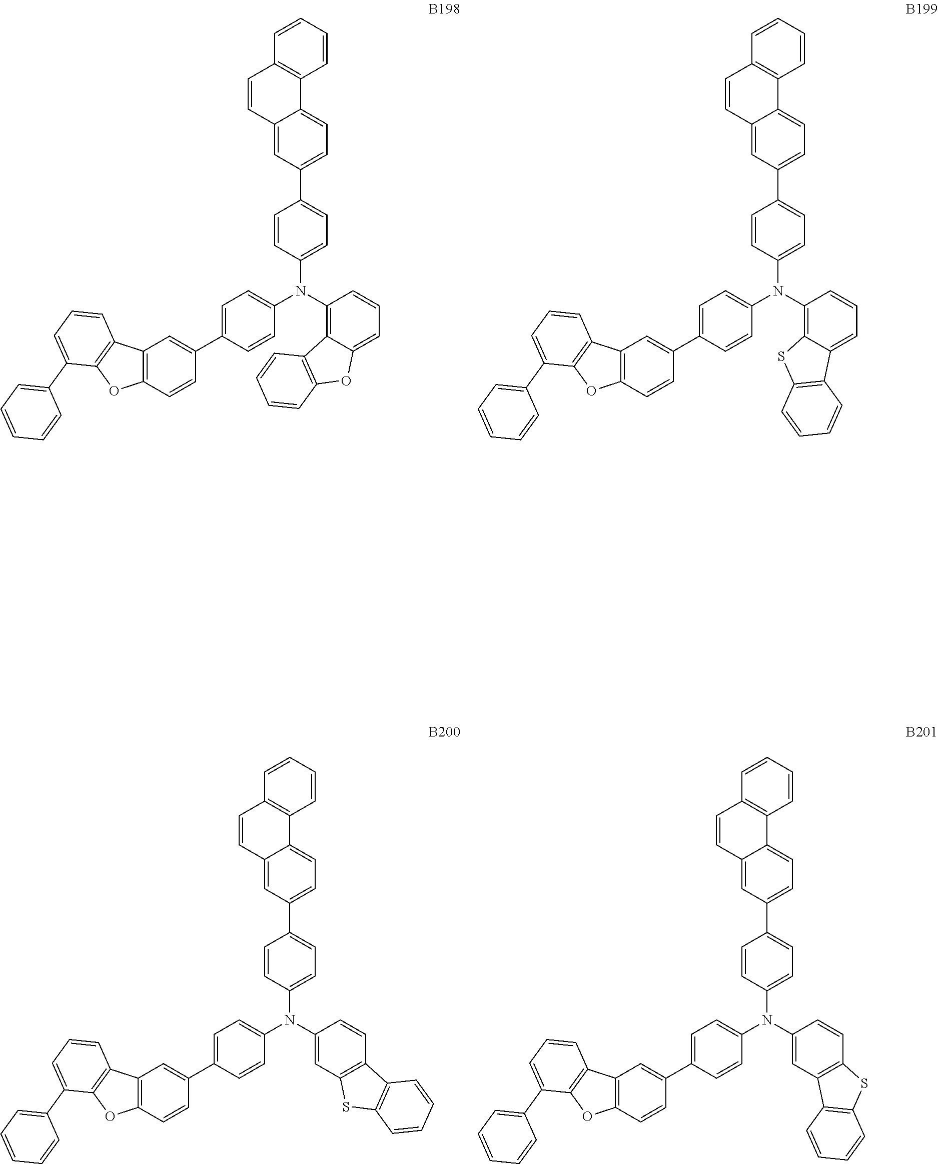 [SMILES: c1ccc(-c2cccc3c2oc2ccc(-c4ccc(N(c5ccc(-c6ccc7c(ccc8ccccc87)c6)cc5)c5cccc6oc7ccccc7c56)cc4)cc23)cc1 ; c1ccc(-c2cccc3c2oc2ccc(-c4ccc(N(c5ccc(-c6ccc7c(ccc8ccccc87)c6)cc5)c5cccc6c5sc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6c(c5)sc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6sc7ccccc7c6c5)cc4)cc23)cc1]

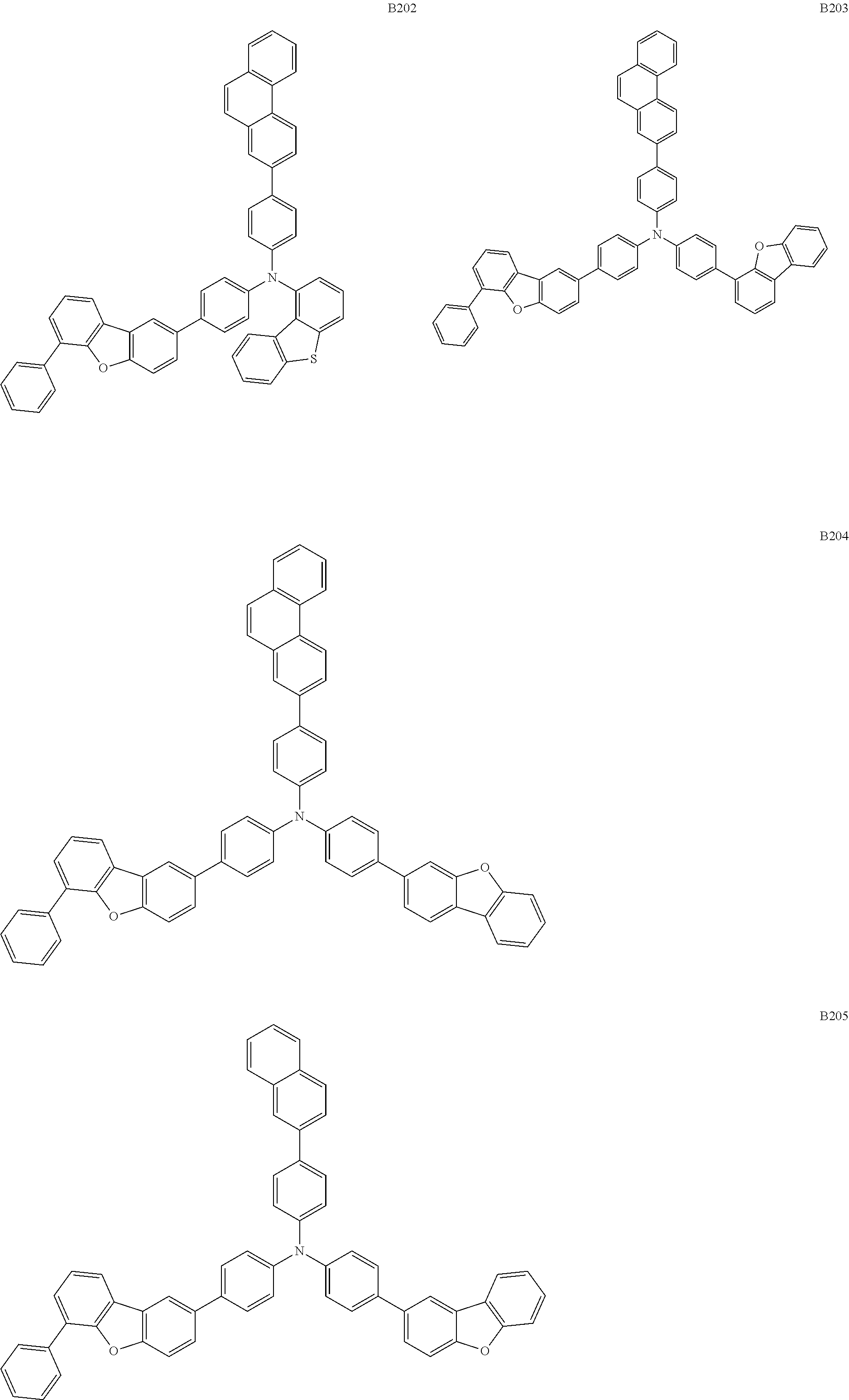 [SMILES: c1ccc(-c2cccc3c2oc2ccc(-c4ccc(N(c5ccc(-c6ccc7c(ccc8ccccc87)c6)cc5)c5cccc6sc7ccccc7c56)cc4)cc23)cc1 ; c1ccc(-c2cccc3c2oc2ccc(-c4ccc(N(c5ccc(-c6ccc7c(ccc8ccccc87)c6)cc5)c5ccc(-c6cccc7c6oc6ccccc67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6ccc7c(c6)oc6ccccc67)cc5)cc4)cc23)cc1 ; c1ccc(-c2cccc3c2oc2ccc(-c4ccc(N(c5ccc(-c6ccc7ccccc7c6)cc5)c5ccc(-c6ccc7oc8ccccc8c7c6)cc5)cc4)cc23)cc1]

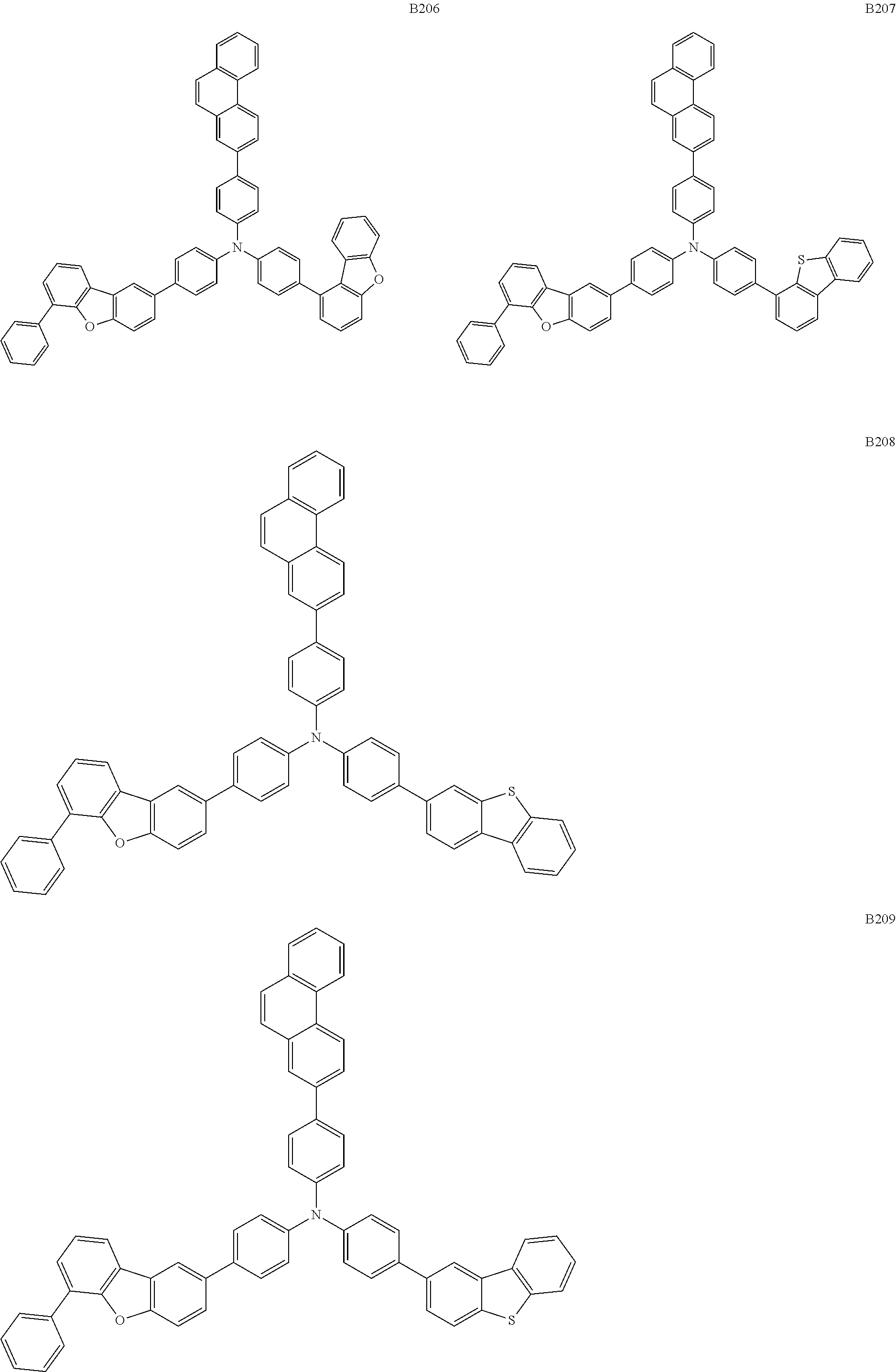 [SMILES: c1ccc(-c2cccc3c2oc2ccc(-c4ccc(N(c5ccc(-c6ccc7c(ccc8ccccc87)c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6ccc7c(c6)sc6ccccc67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6ccc7sc8ccccc8c7c6)cc5)cc4)cc23)cc1]

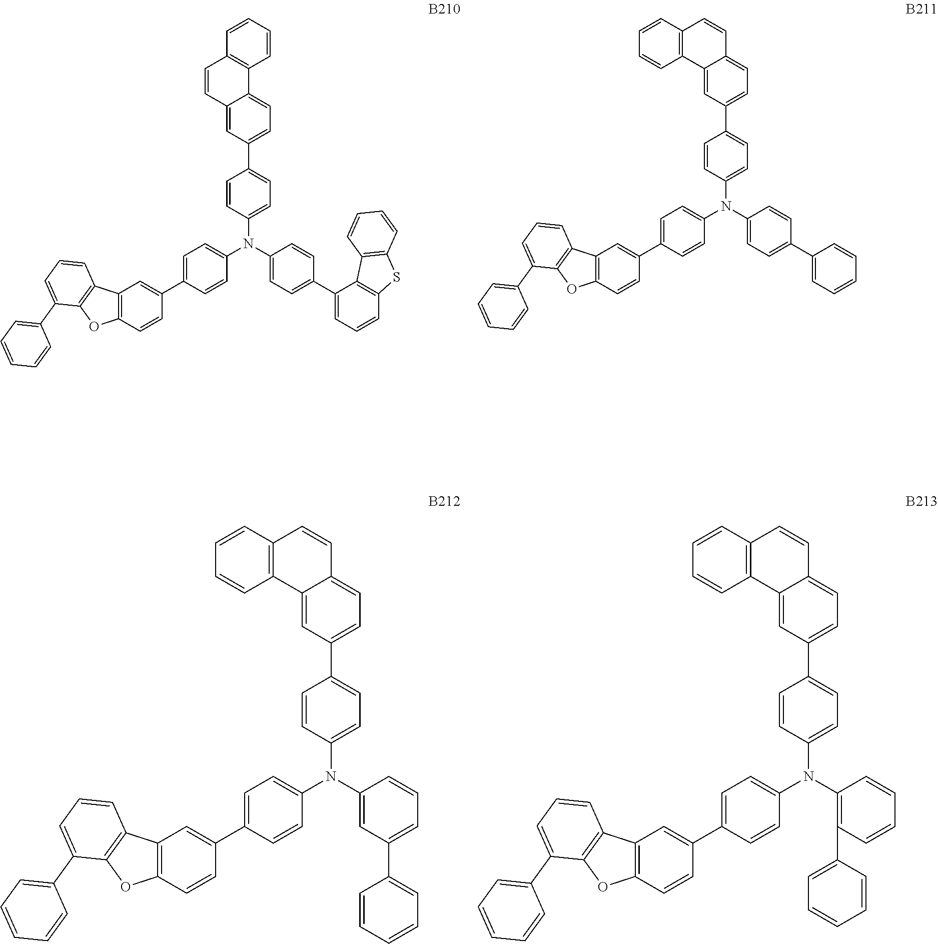 [SMILES: c1ccc(-c2cccc3c2oc2ccc(-c4ccc(N(c5ccc(-c6ccc7c(ccc8ccccc87)c6)cc5)c5ccc(-c6cccc7sc8ccccc8c67)cc5)cc4)cc23)cc1 ; c1ccc(-c2ccc(N(c3ccc(-c4ccc5ccc6ccccc6c5c4)cc3)c3ccc(-c4ccc5oc6c(-c7ccccc7)cccc6c5c4)cc3)cc2)cc1 ; c1ccc(-c2cccc(N(c3ccc(-c4ccc5ccc6ccccc6c5c4)cc3)c3ccc(-c4ccc5oc6c(-c7ccccc7)cccc6c5c4)cc3)c2)cc1 ; c1ccc(-c2ccccc2N(c2ccc(-c3ccc4ccc5ccccc5c4c3)cc2)c2ccc(-c3ccc4oc5c(-c6ccccc6)cccc5c4c3)cc2)cc1]

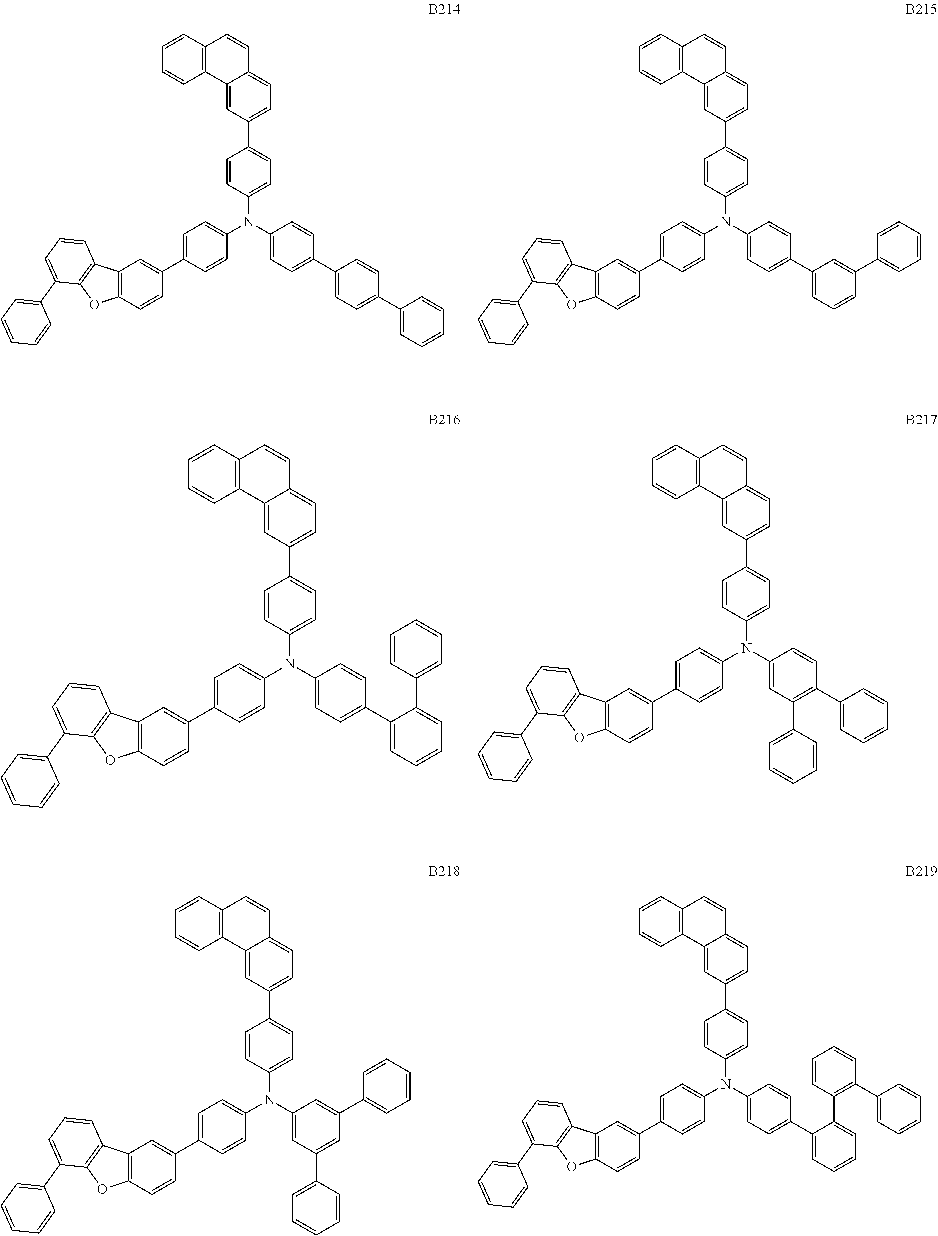

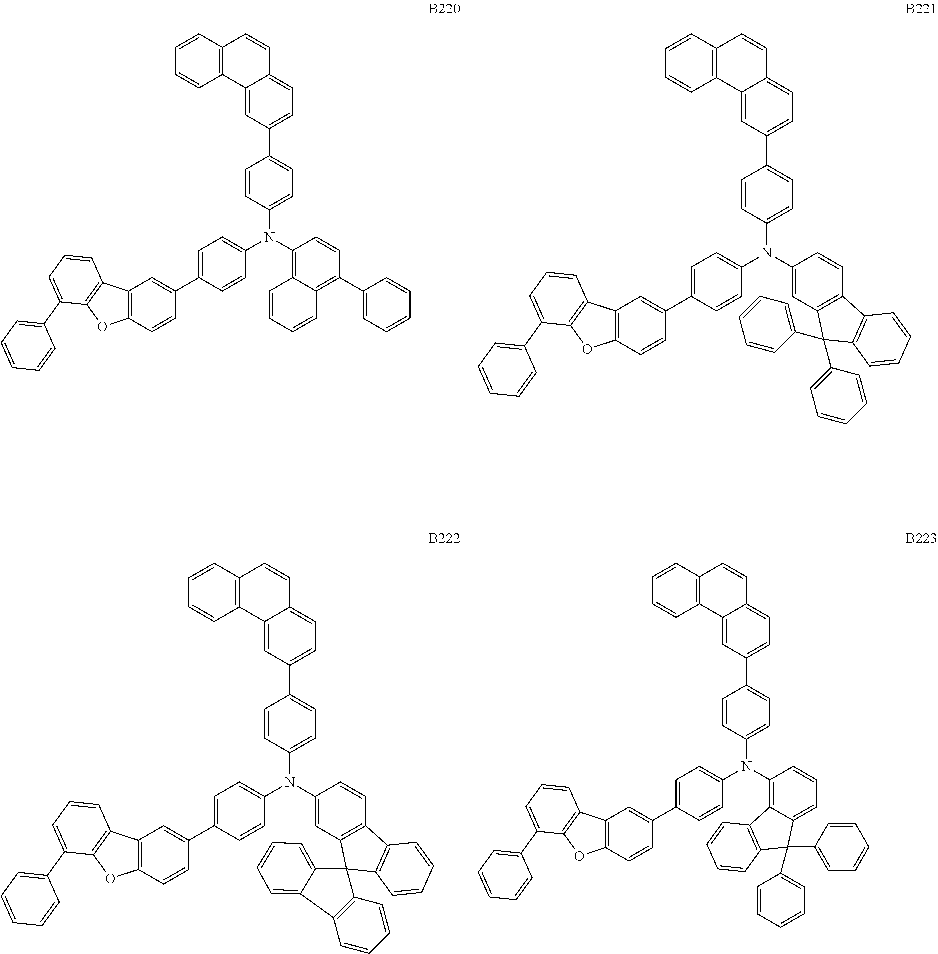 [SMILES: c1ccc(-c2ccc(N(c3ccc(-c4ccc5ccc6ccccc6c5c4)cc3)c3ccc(-c4ccc5oc6c(-c7ccccc7)cccc6c5c4)cc3)c3ccccc23)cc1 ; c1ccc(-c2cccc3c2oc2ccc(-c4ccc(N(c5ccc(-c6ccc7ccc8ccccc8c7c6)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6ccc7ccc8ccccc8c7c6)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6ccc7ccc8ccccc8c7c6)cc5)c5cccc6c5-c5ccccc5C6(c5ccccc5)c5ccccc5)cc4)cc23)cc1]

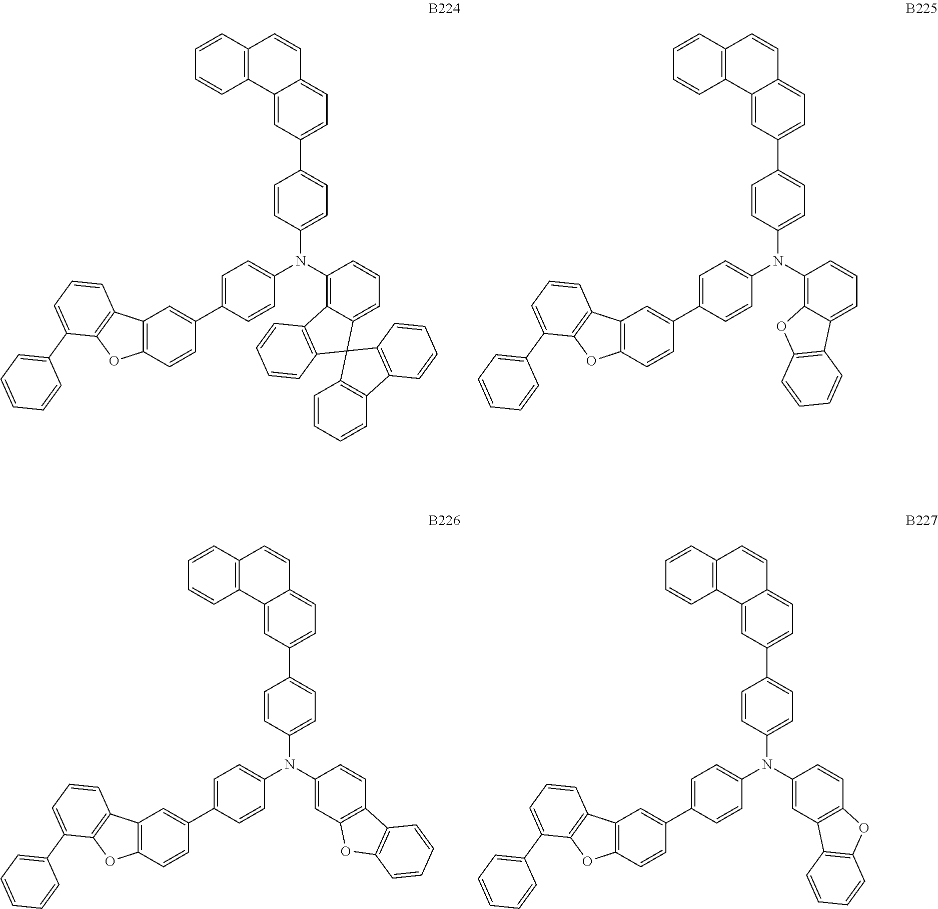 [SMILES: c1ccc(-c2cccc3c2oc2ccc(-c4ccc(N(c5ccc(-c6ccc7ccc8ccccc8c7c6)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6ccc7ccc8ccccc8c7c6)cc5)c5cccc6c5oc5ccccc56)cc4)cc23)cc1 ; c1ccc(-c2cccc3c2oc2ccc(-c4ccc(N(c5ccc(-c6ccc7ccc8ccccc8c7c6)cc5)c5ccc6c(c5)oc5ccccc56)cc4)cc23)cc1 ; c1ccc(-c2cccc3c2oc2ccc(-c4ccc(N(c5ccc(-c6ccc7ccc8ccccc8c7c6)cc5)c5ccc6oc7ccccc7c6c5)cc4)cc23)cc1]

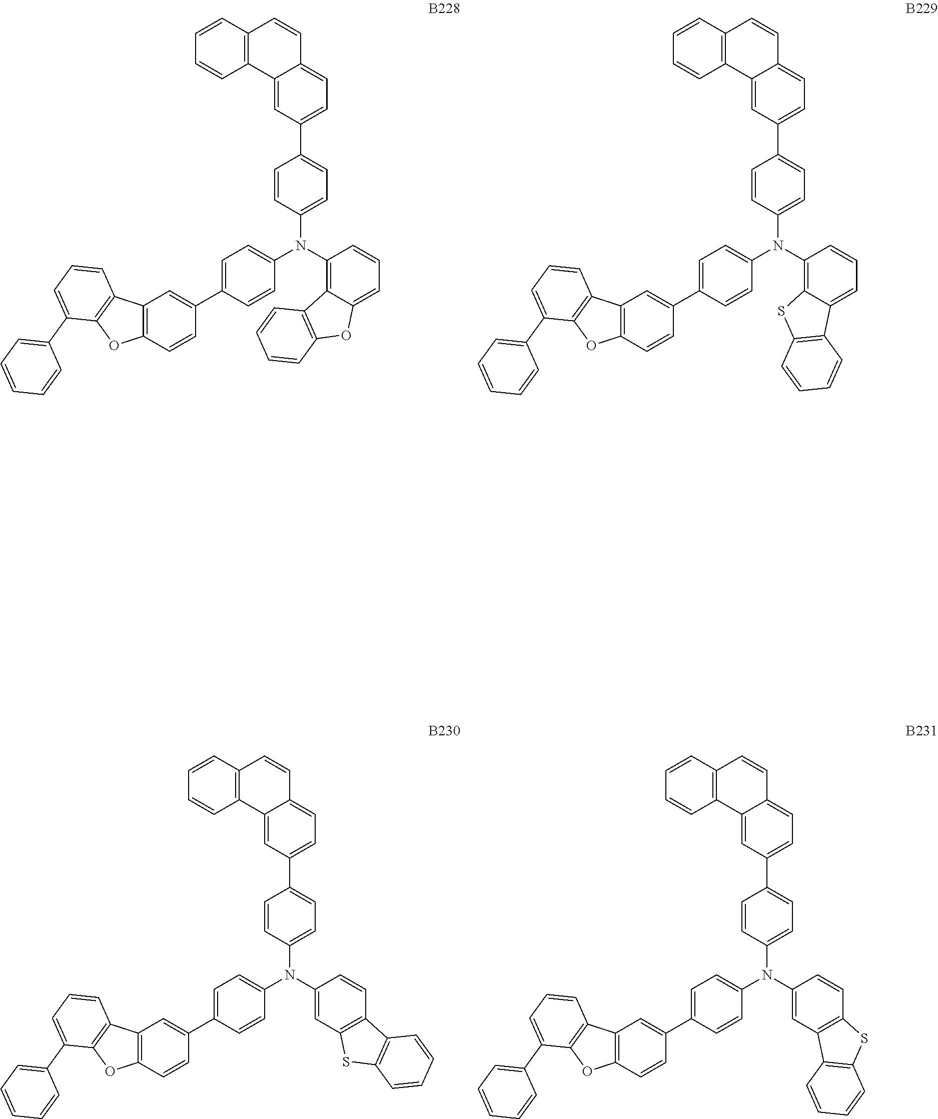 [SMILES: c1ccc(-c2cccc3c2oc2ccc(-c4ccc(N(c5ccc(-c6ccc7ccc8ccccc8c7c6)cc5)c5cccc6oc7ccccc7c56)cc4)cc23)cc1 ; c1ccc(-c2cccc3c2oc2ccc(-c4ccc(N(c5ccc(-c6ccc7ccc8ccccc8c7c6)cc5)c5cccc6c5sc5ccccc56)cc4)cc23)cc1 ; c1ccc(-c2cccc3c2oc2ccc(-c4ccc(N(c5ccc(-c6ccc7ccc8ccccc8c7c6)cc5)c5ccc6c(c5)sc5ccccc56)cc4)cc23)cc1 ; c1ccc(-c2cccc3c2oc2ccc(-c4ccc(N(c5ccc(-c6ccc7ccc8ccccc8c7c6)cc5)c5ccc6sc7ccccc7c6c5)cc4)cc23)cc1]

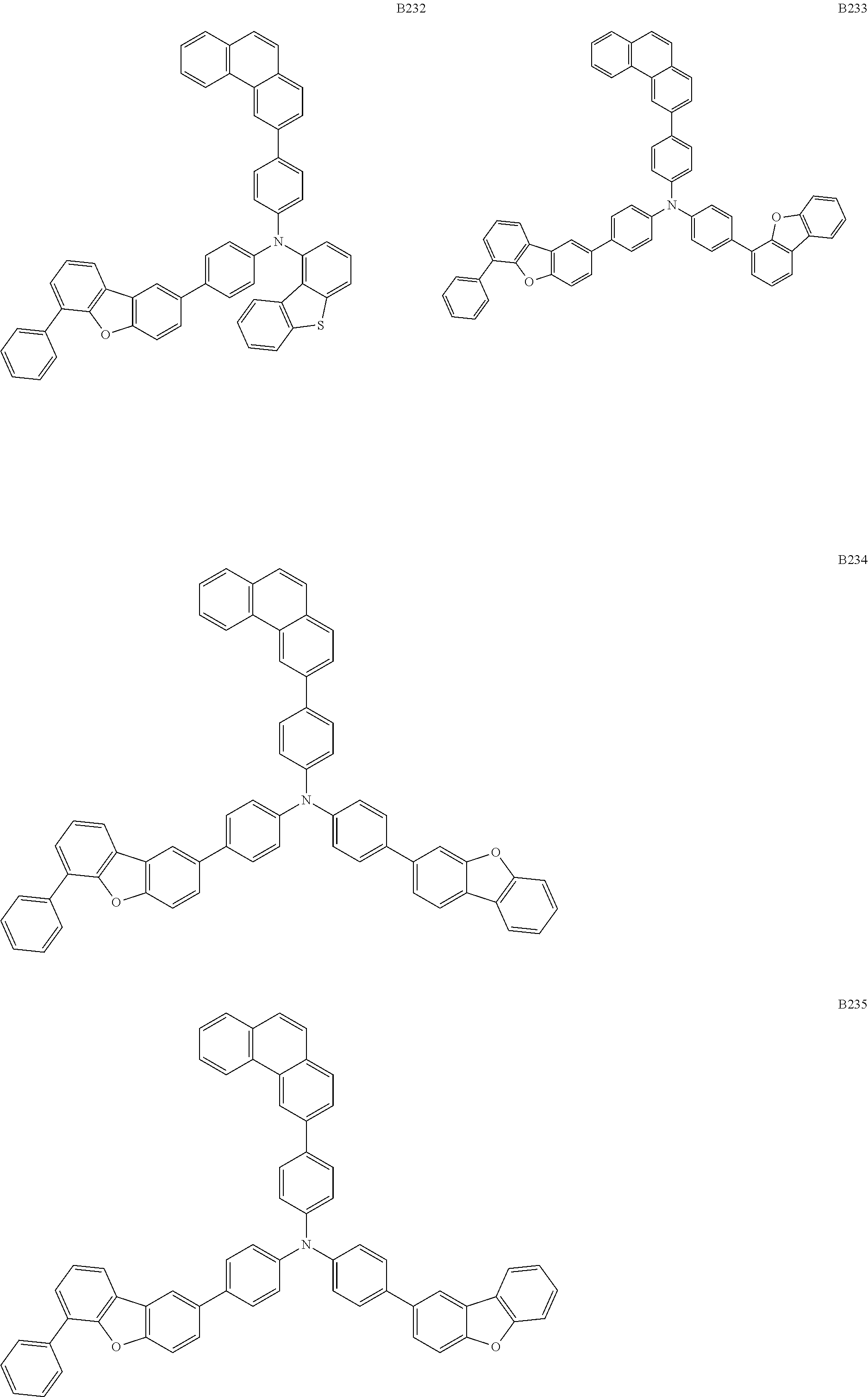 [SMILES: c1ccc(-c2cccc3c2oc2ccc(-c4ccc(N(c5ccc(-c6ccc7ccc8ccccc8c7c6)cc5)c5cccc6sc7ccccc7c56)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7c6oc6ccccc67)cc5)cc4)cc23)cc1 ; c1ccc(-c2cccc3c2oc2ccc(-c4ccc(N(c5ccc(-c6ccc7c(c6)oc6ccccc67)cc5)c5ccc(-c6ccc7ccc8ccccc8c7c6)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6ccc7oc8ccccc8c7c6)cc5)cc4)cc23)cc1]

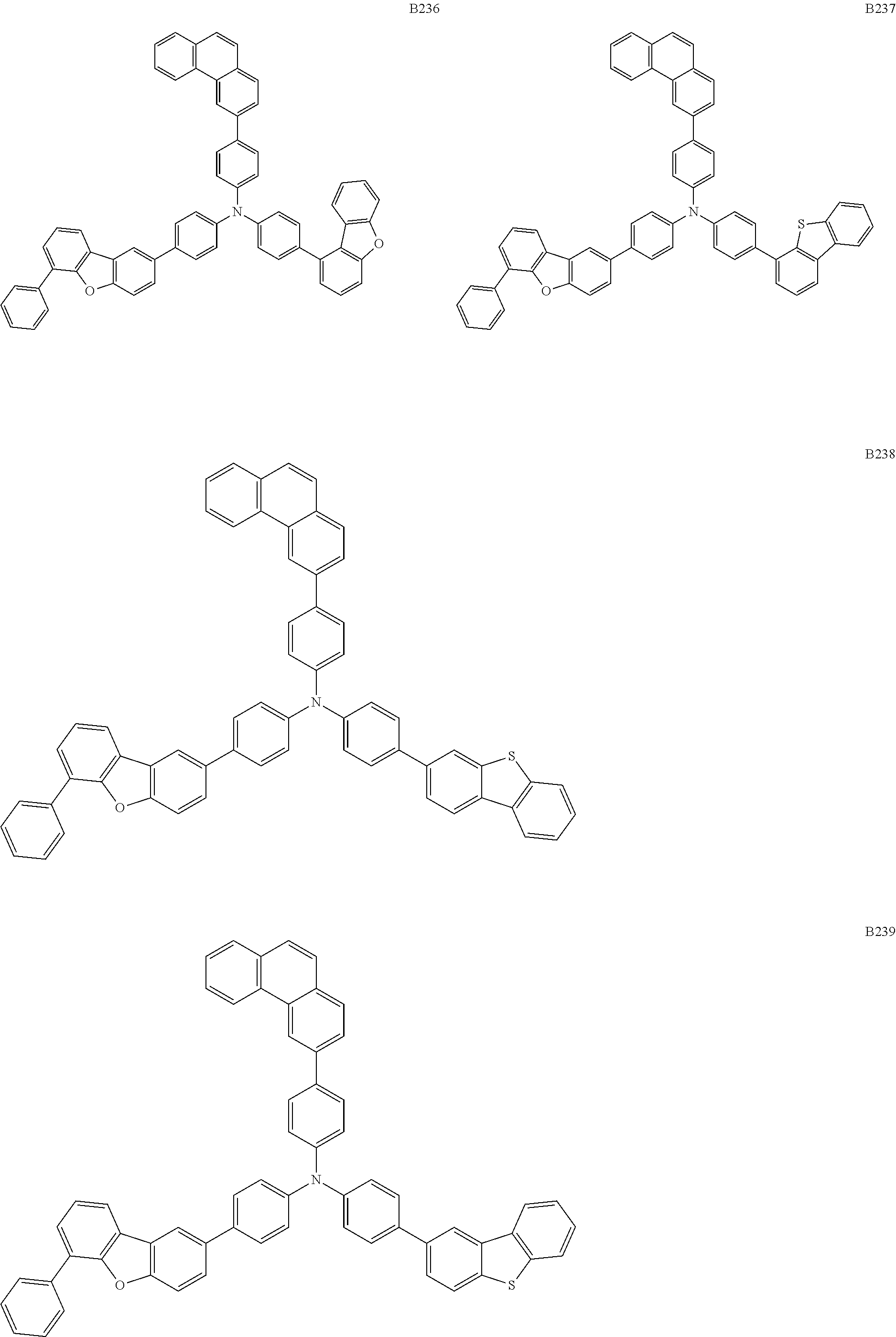 [SMILES: c1ccc(-c2cccc3c2oc2ccc(-c4ccc(N(c5ccc(-c6ccc7ccc8ccccc8c7c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6ccc7ccc8ccccc8c7c6)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6ccc7sc8ccccc8c7c6)cc5)cc4)cc23)cc1]

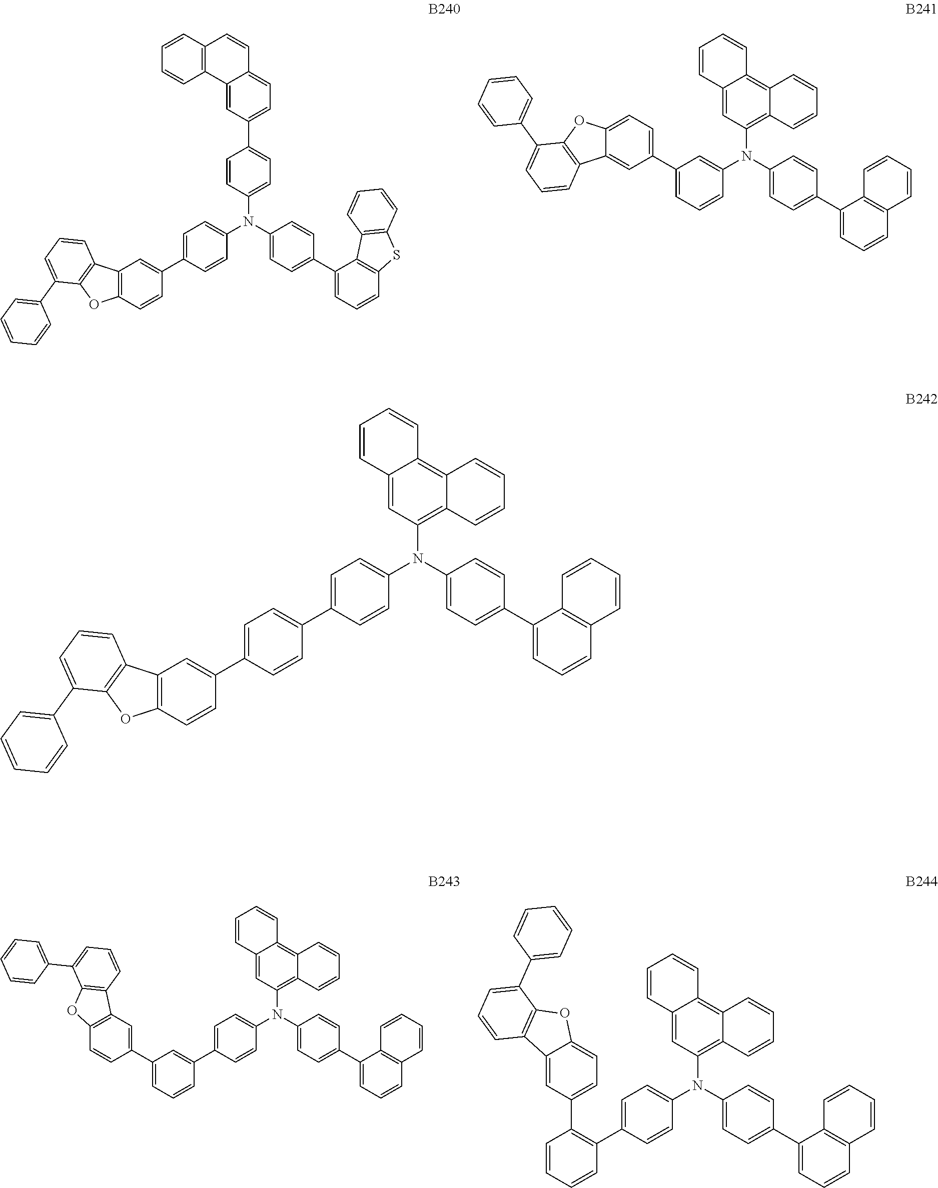

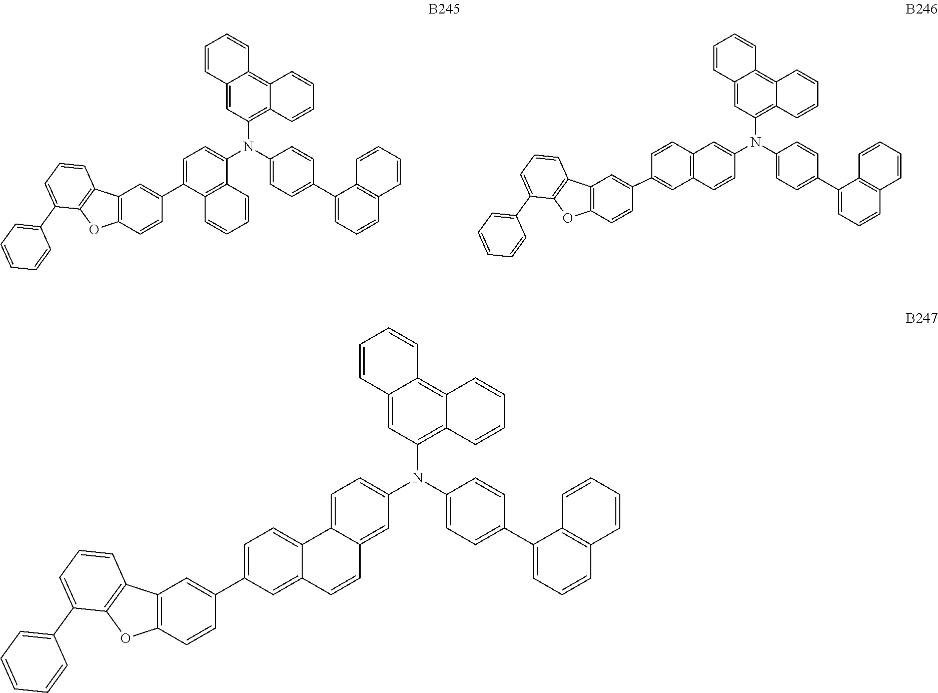 [SMILES: c1ccc(-c2cccc3c2oc2ccc(-c4ccc(N(c5ccc(-c6cccc7ccccc67)cc5)c5cc6ccccc6c6ccccc56)c5ccccc45)cc23)cc1 ; c1ccc(-c2cccc3c2oc2ccc(-c4ccc5cc(N(c6ccc(-c7cccc8ccccc78)cc6)c6cc7ccccc7c7ccccc67)ccc5c4)cc23)cc1 ; c1ccc(-c2cccc3c2oc2ccc(-c4ccc5c(ccc6cc(N(c7ccc(-c8cccc9ccccc89)cc7)c7cc8ccccc8c8ccccc78)ccc65)c4)cc23)cc1]

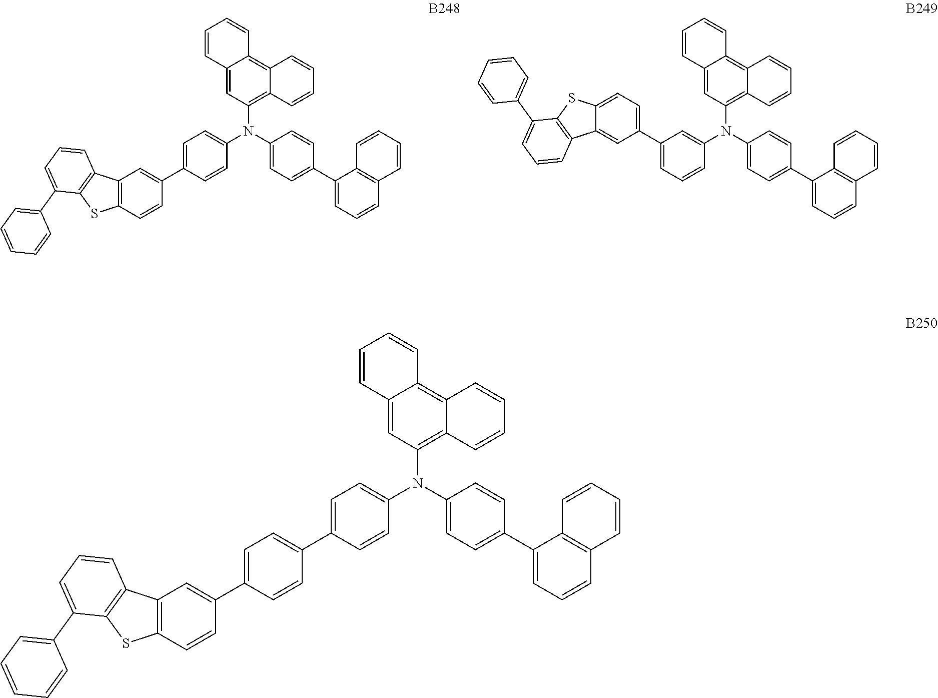 [SMILES: c1ccc(-c2cccc3c2sc2ccc(-c4ccc(N(c5ccc(-c6cccc7ccccc67)cc5)c5cc6ccccc6c6ccccc56)cc4)cc23)cc1 ; c1ccc(-c2cccc3c2sc2ccc(-c4cccc(N(c5ccc(-c6cccc7ccccc67)cc5)c5cc6ccccc6c6ccccc56)c4)cc23)cc1 ; c1ccc(-c2cccc3c2sc2ccc(-c4ccc(-c5ccc(N(c6ccc(-c7cccc8ccccc78)cc6)c6cc7ccccc7c7ccccc67)cc5)cc4)cc23)cc1]

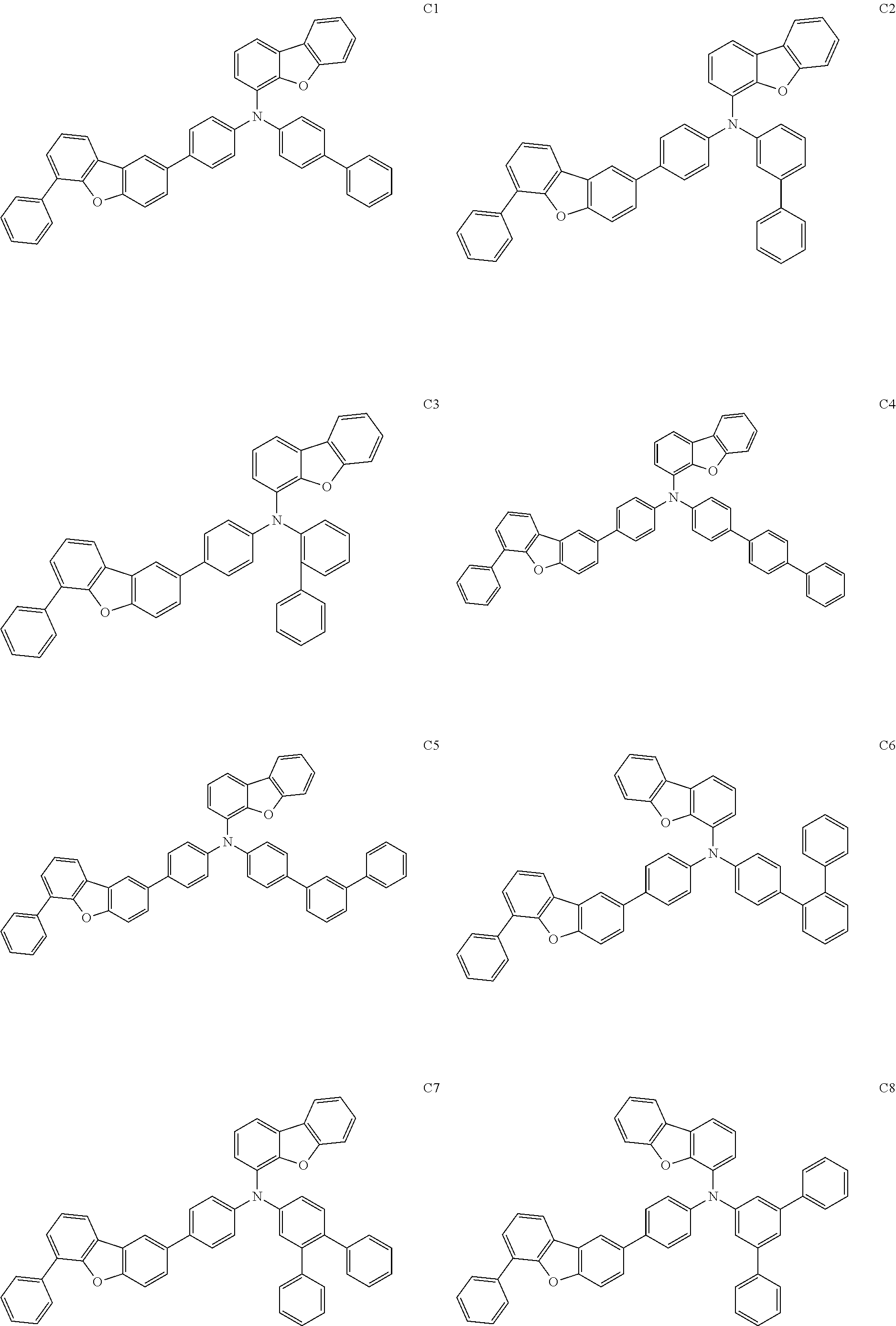

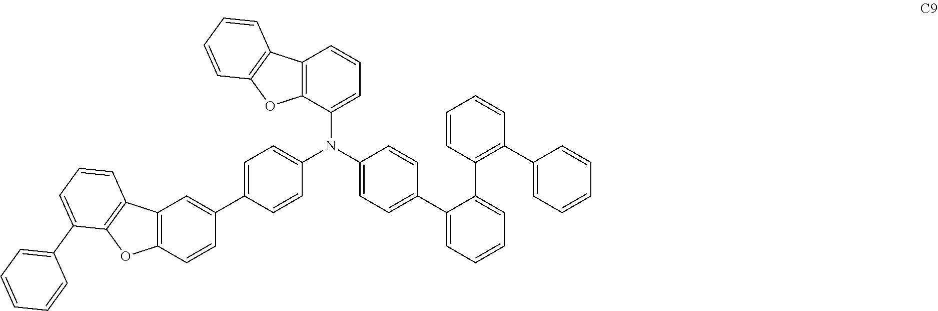 [SMILES: c1ccc(-c2ccccc2-c2ccccc2-c2ccc(N(c3ccc(-c4ccc5oc6c(-c7ccccc7)cccc6c5c4)cc3)c3cccc4c3oc3ccccc34)cc2)cc1]

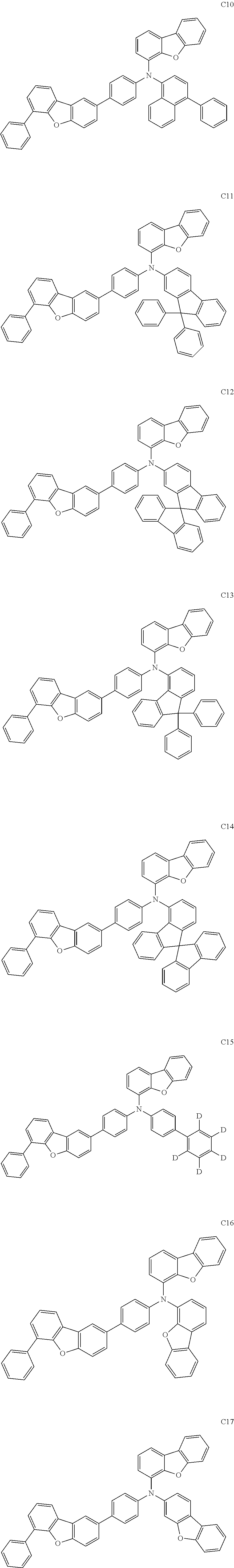

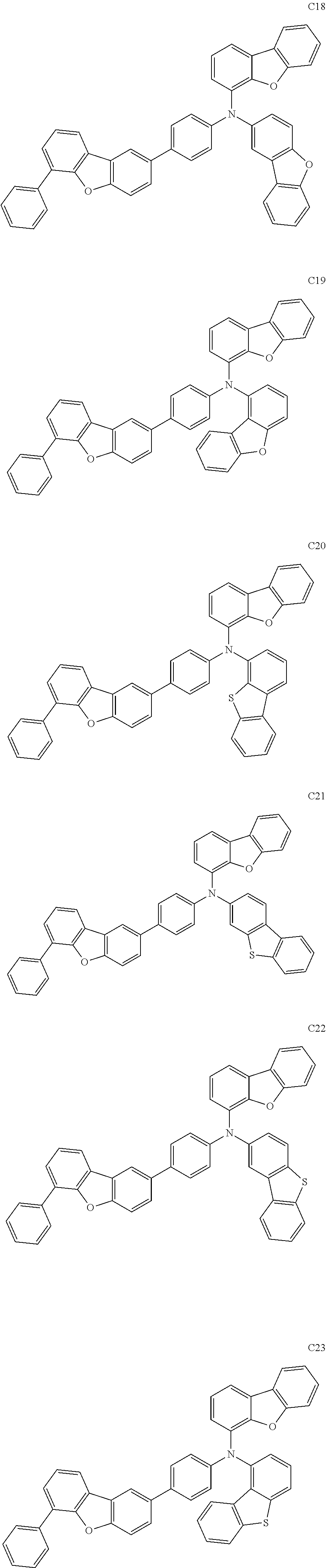

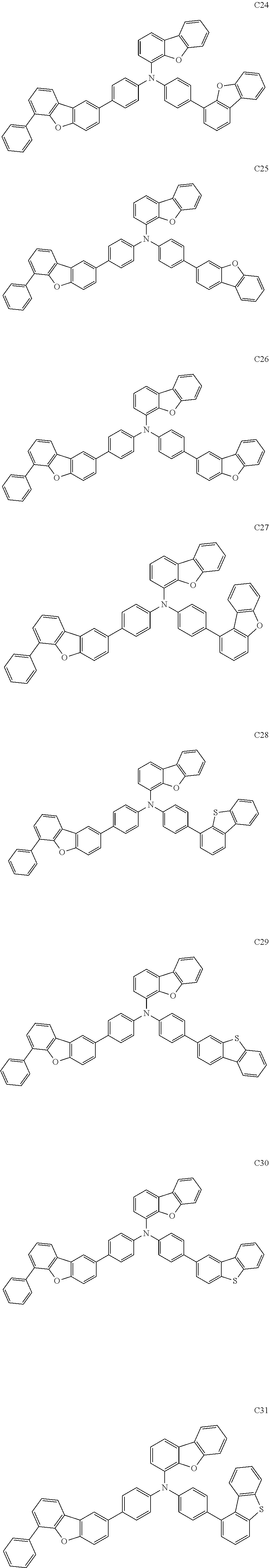

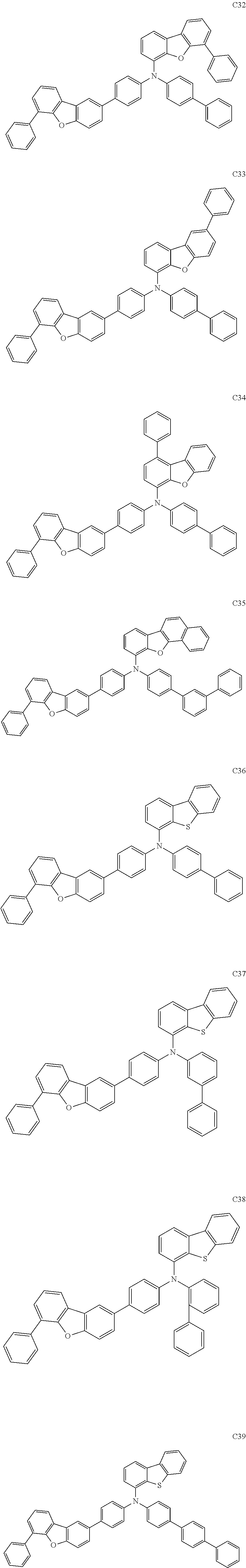

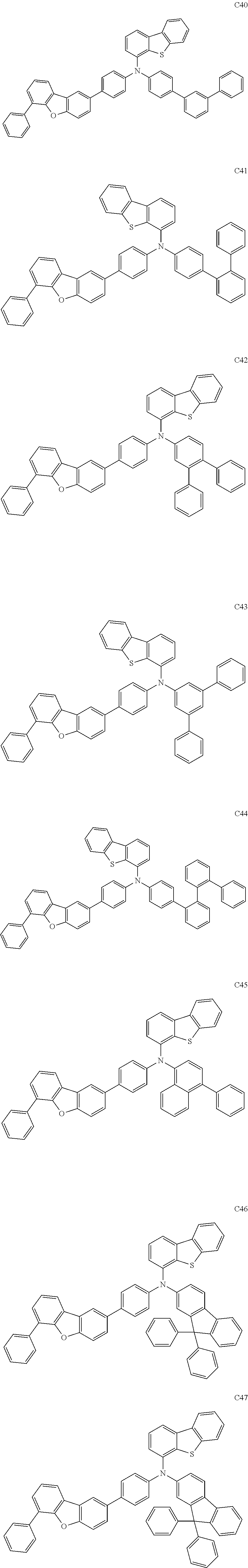

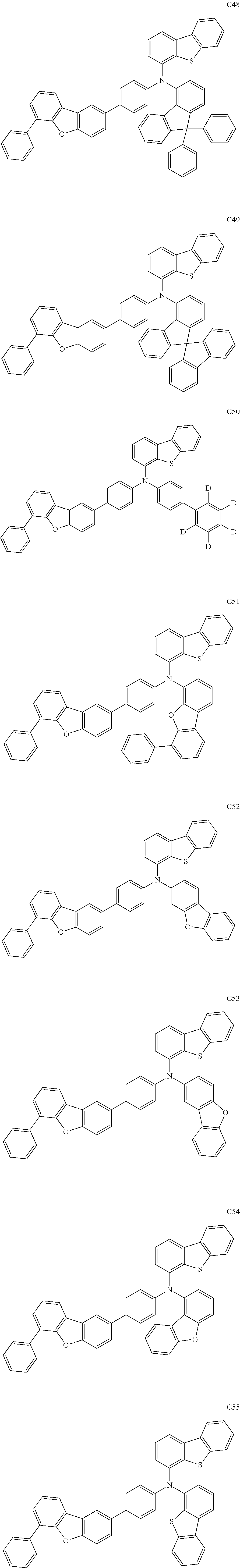

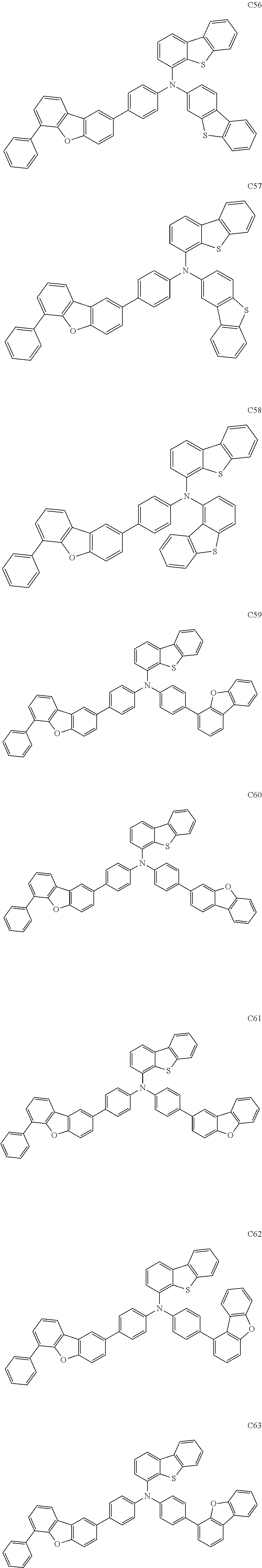

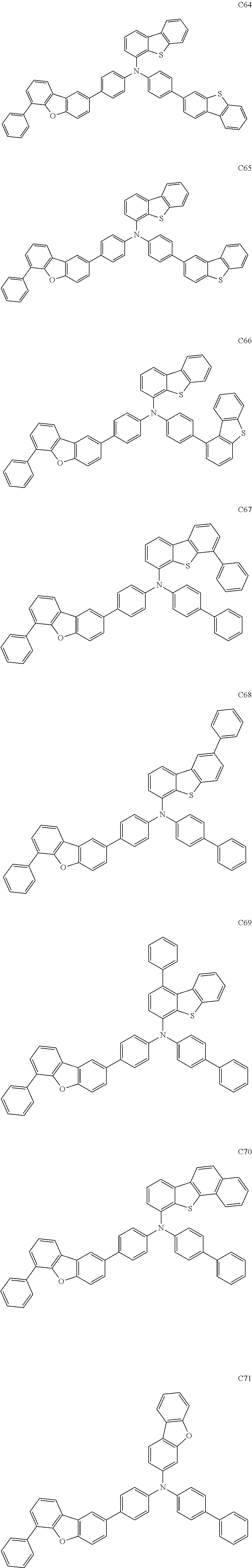

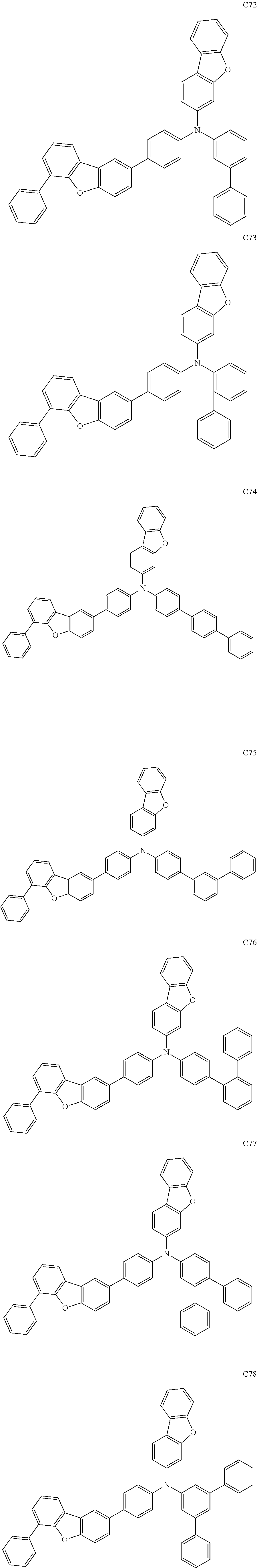

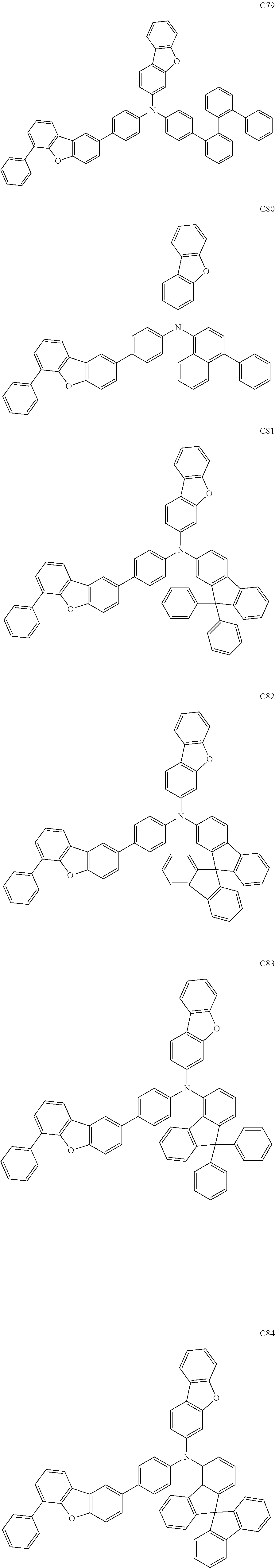

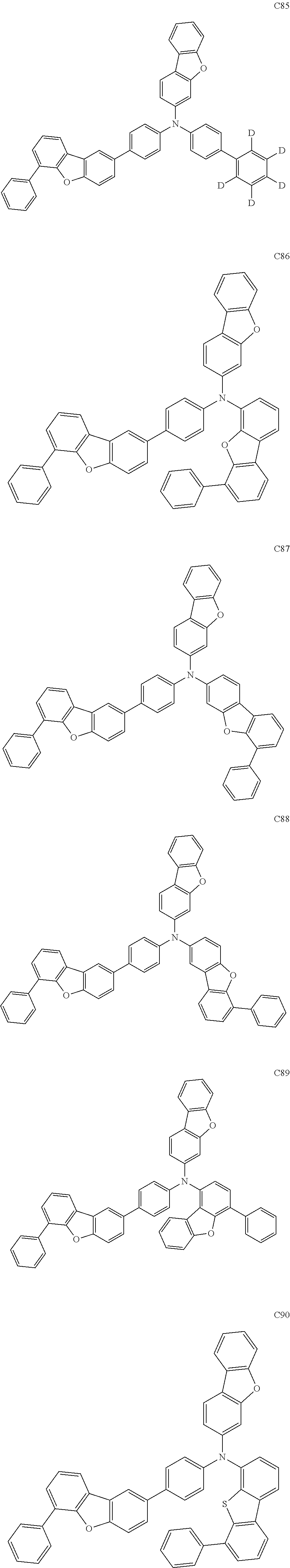

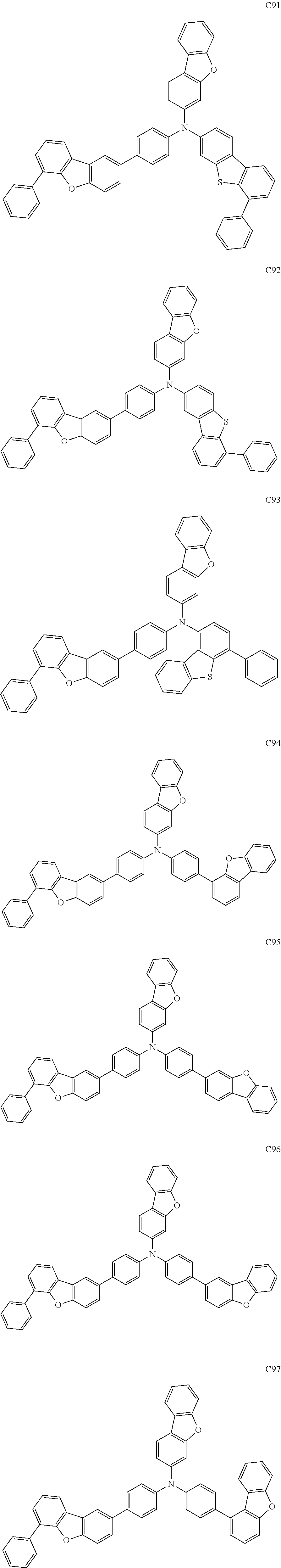

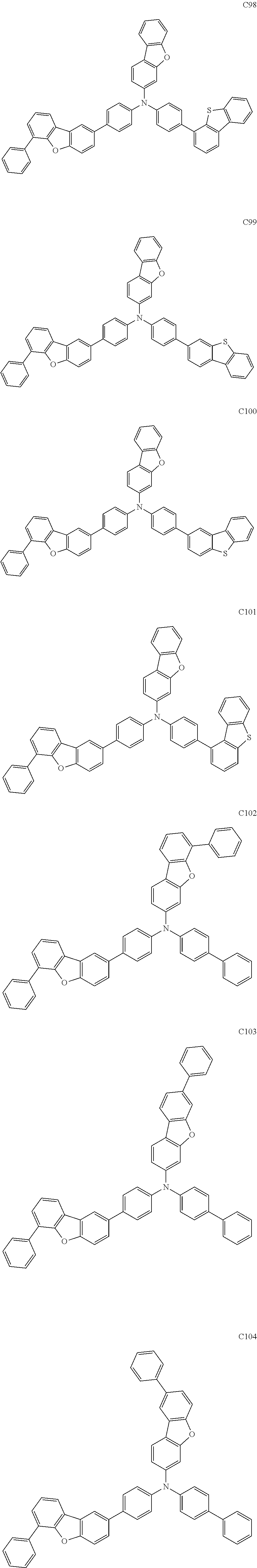

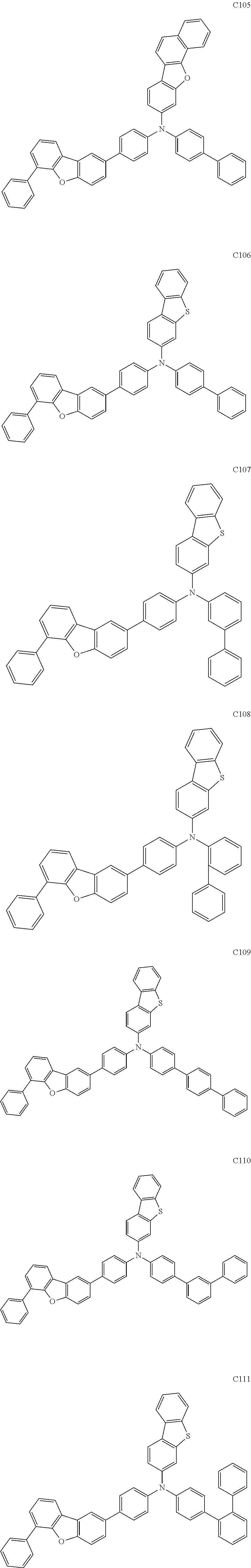

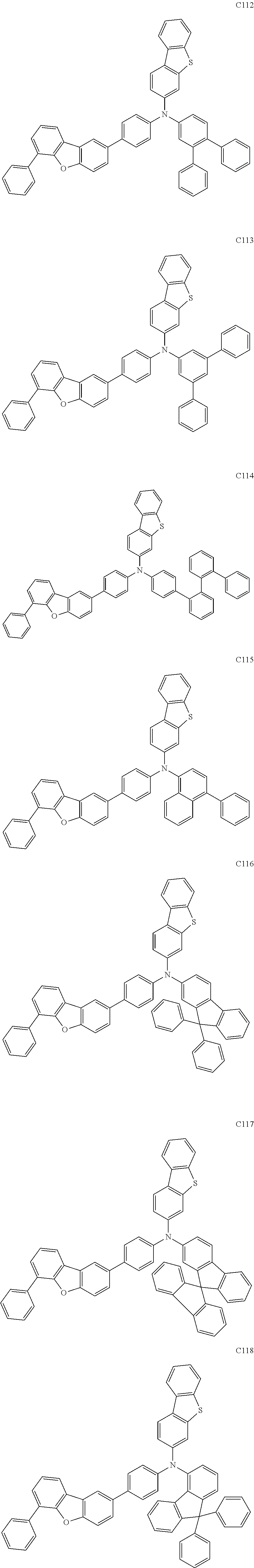

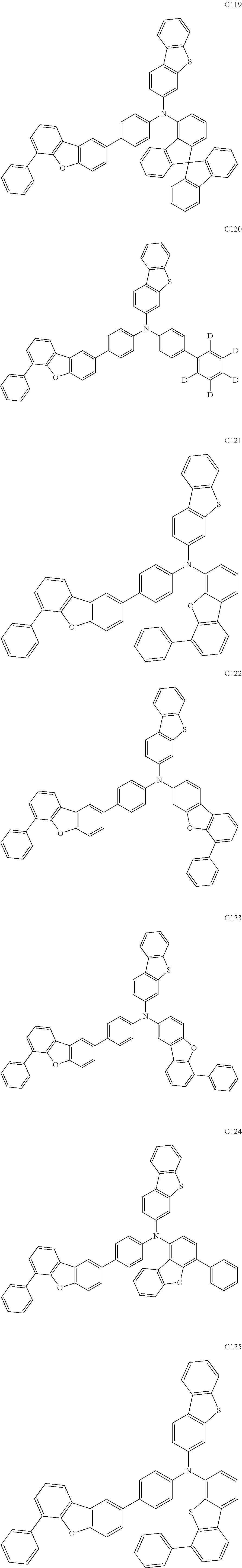

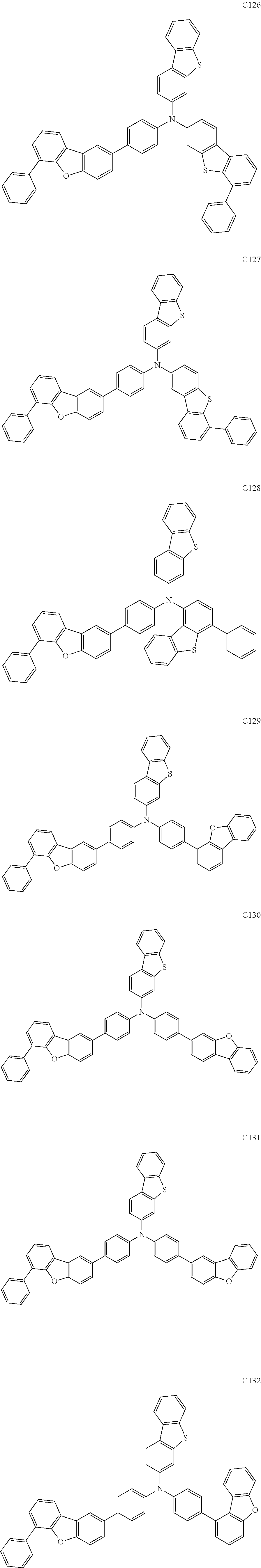

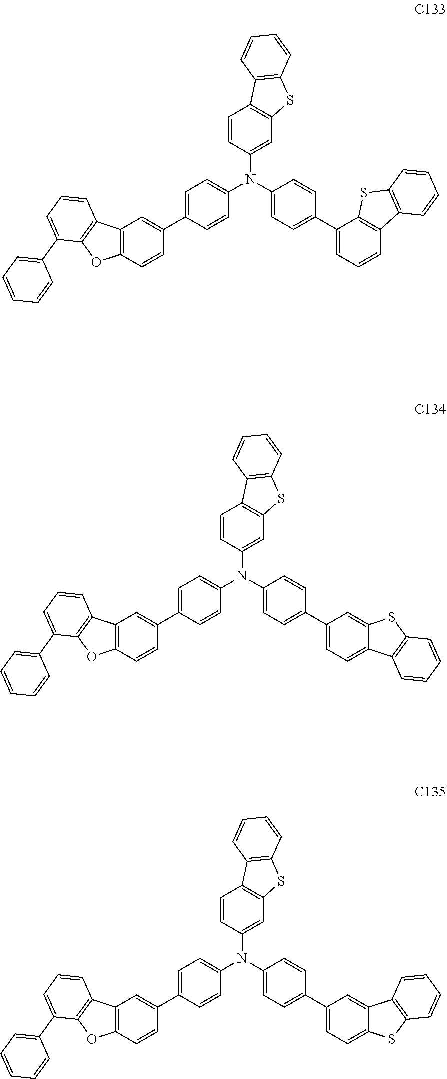 [SMILES: c1ccc(-c2cccc3c2oc2ccc(-c4ccc(N(c5ccc(-c6cccc7c6sc6ccccc67)cc5)c5ccc6c(c5)sc5ccccc56)cc4)cc23)cc1 ; c1ccc(-c2cccc3c2oc2ccc(-c4ccc(N(c5ccc(-c6ccc7c(c6)sc6ccccc67)cc5)c5ccc6c(c5)sc5ccccc56)cc4)cc23)cc1 ; c1ccc(-c2cccc3c2oc2ccc(-c4ccc(N(c5ccc(-c6ccc7sc8ccccc8c7c6)cc5)c5ccc6c(c5)sc5ccccc56)cc4)cc23)cc1]

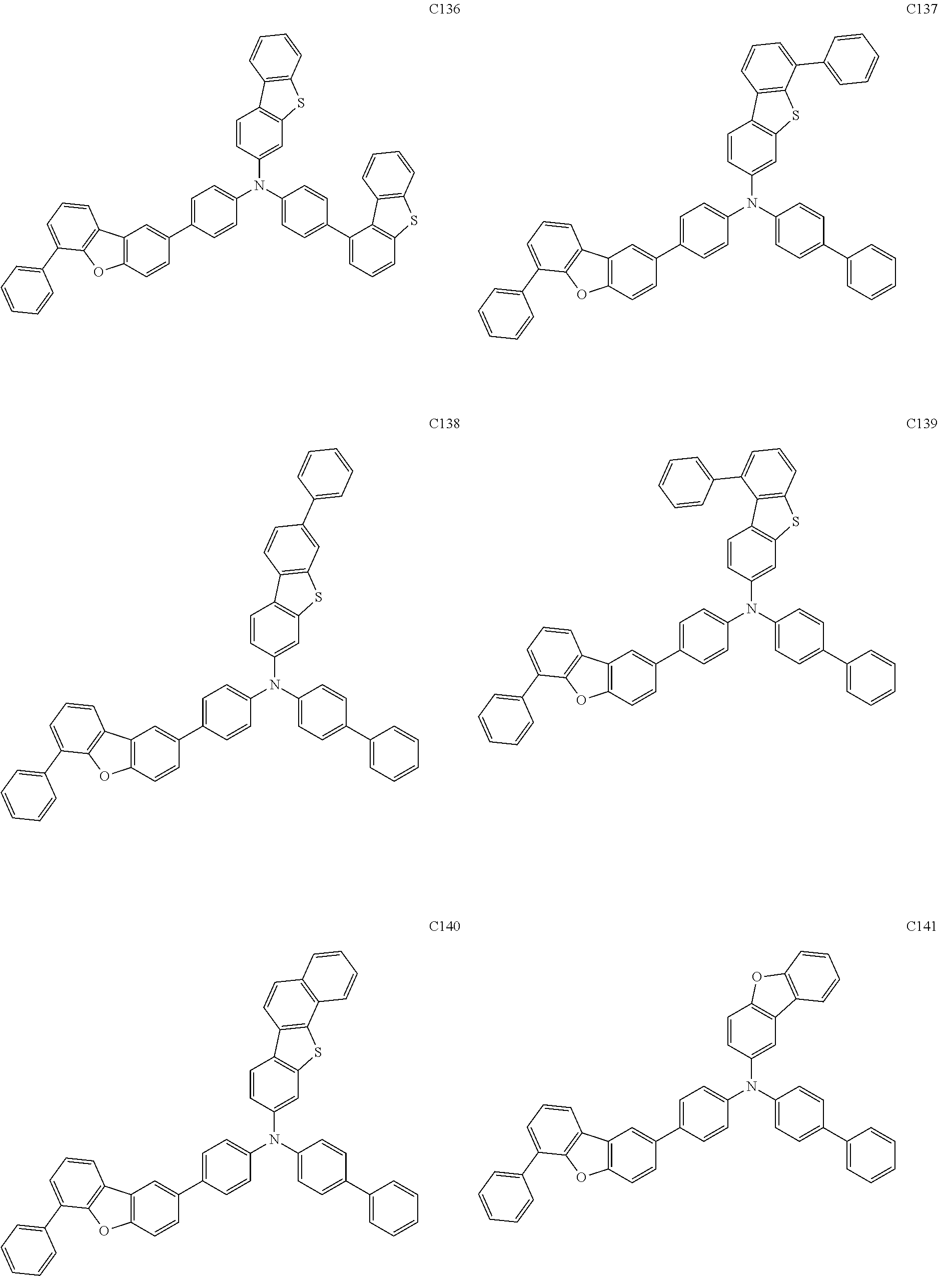

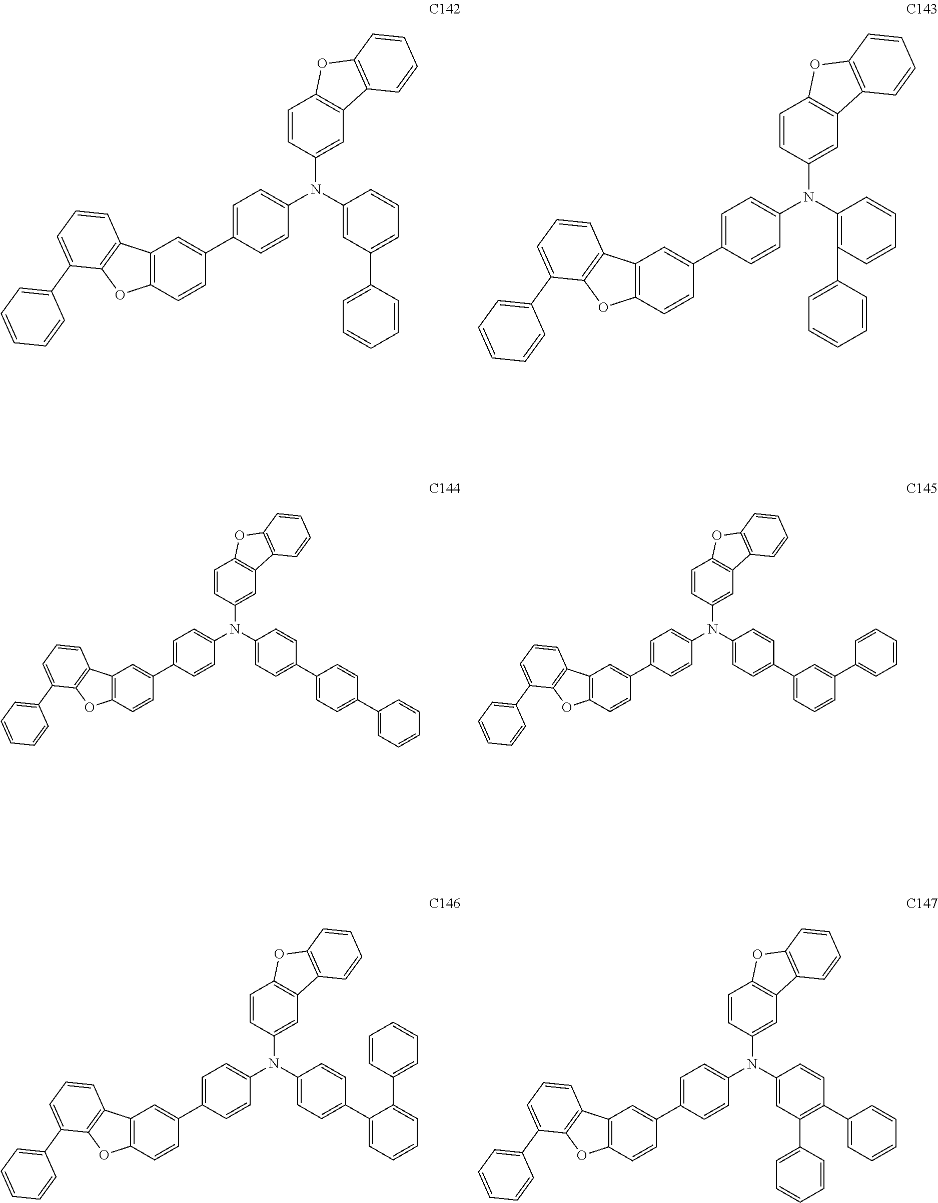

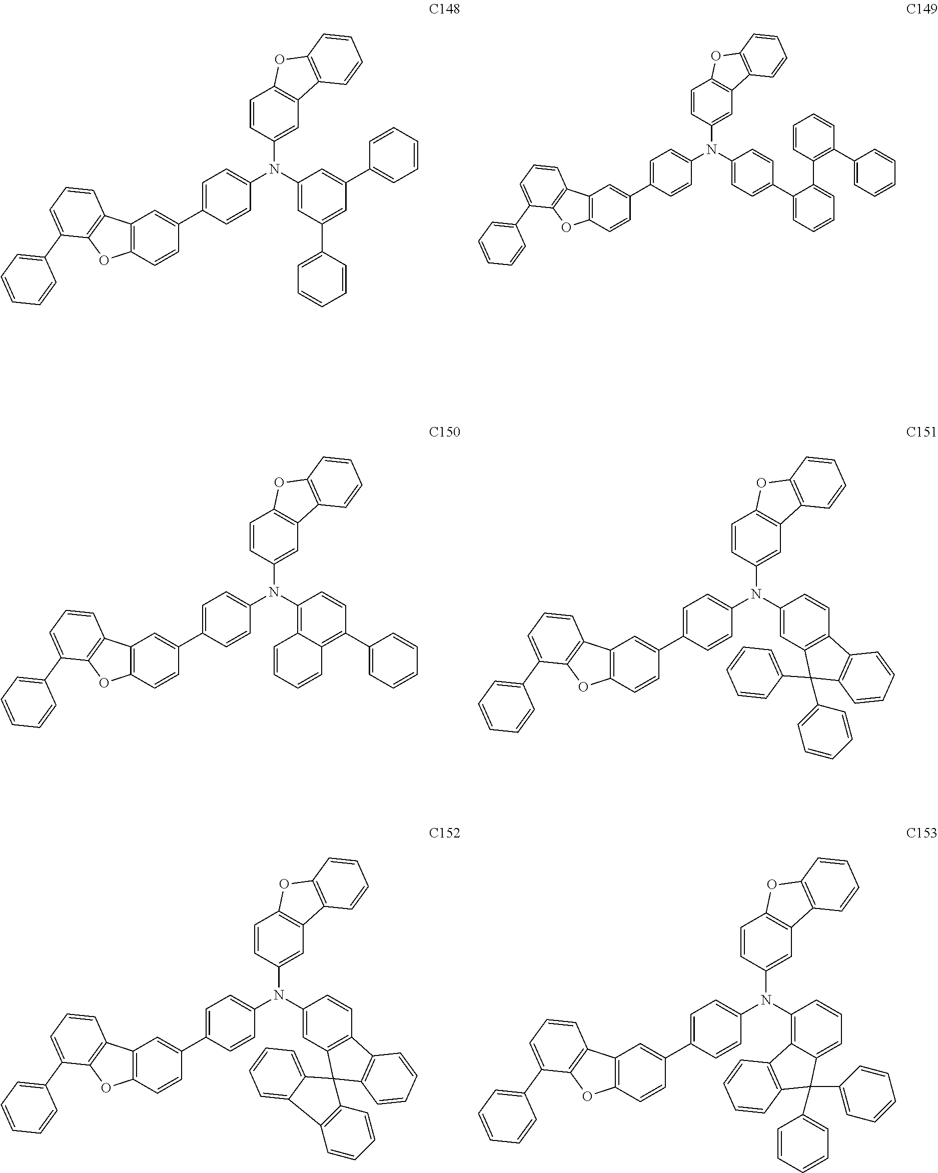

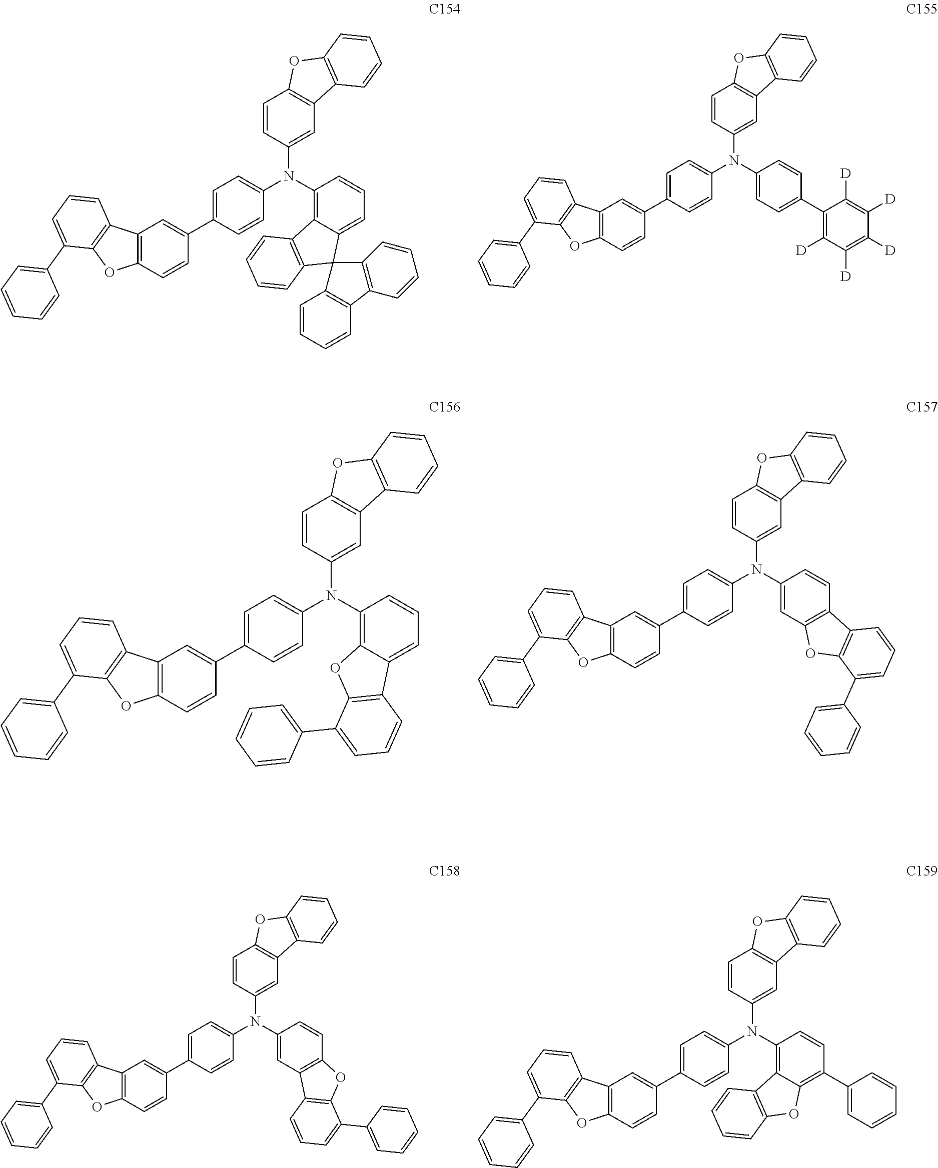

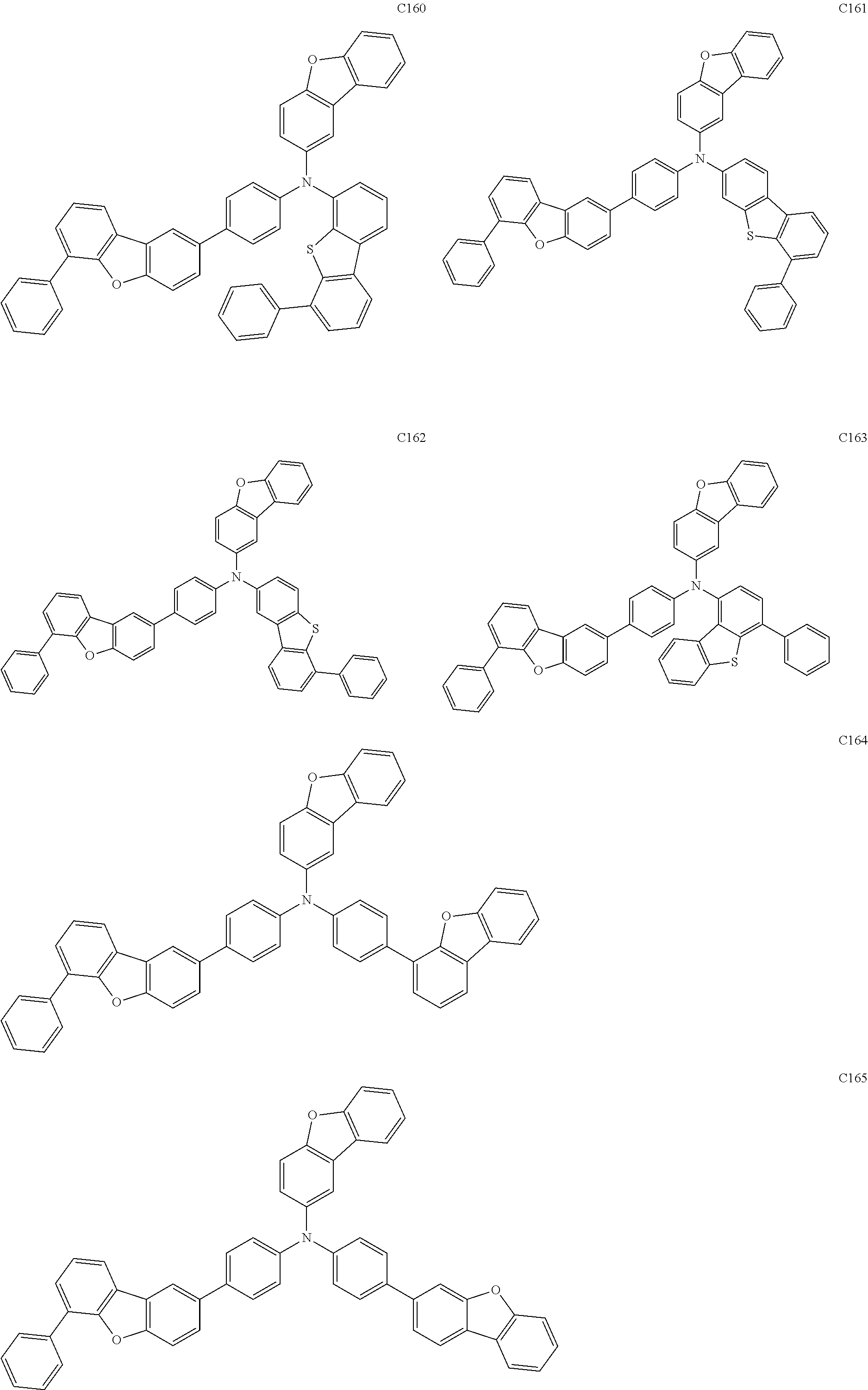

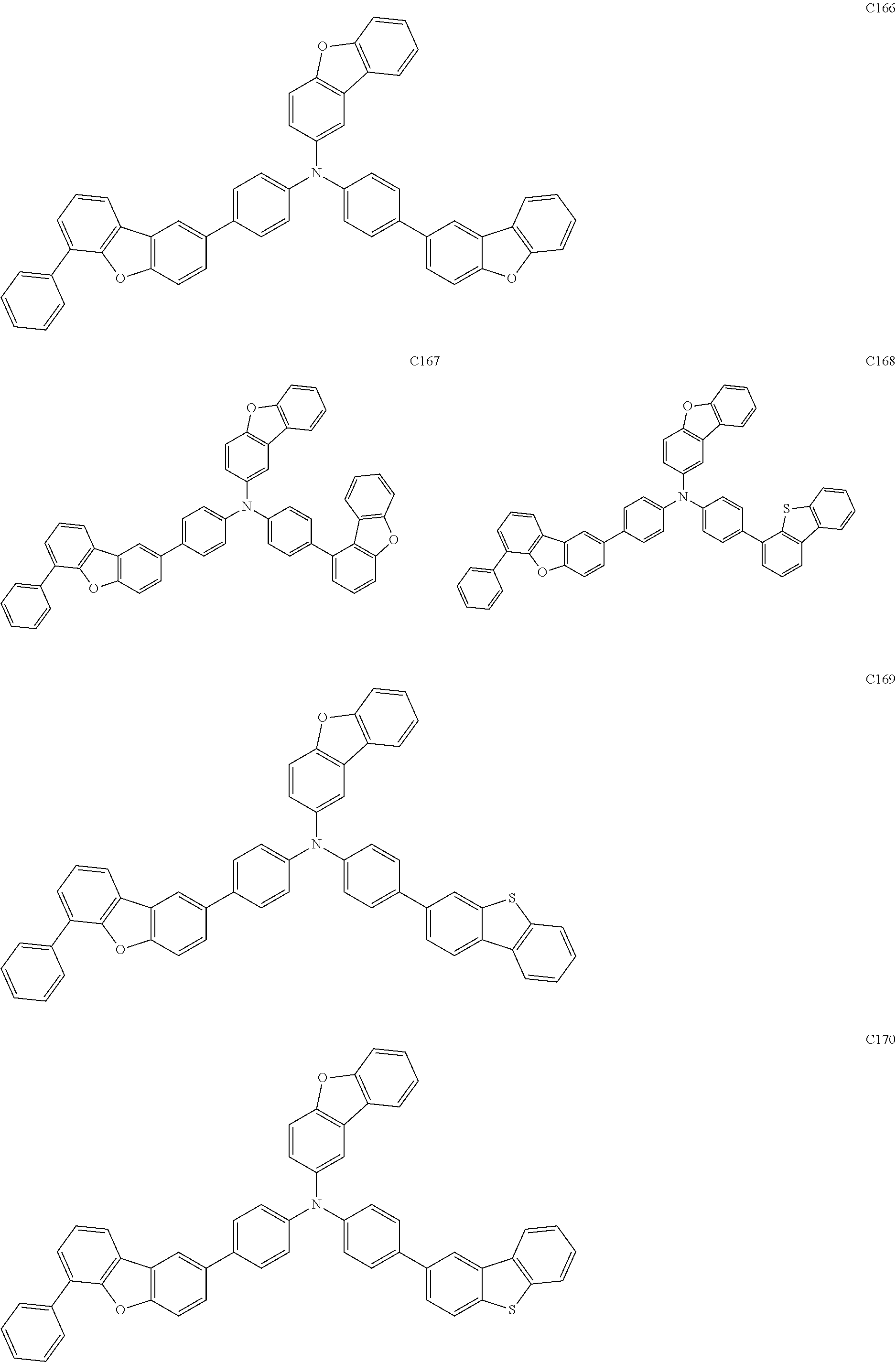 [SMILES: c1ccc(-c2cccc3c2oc2ccc(-c4ccc(N(c5ccc(-c6ccc7oc8ccccc8c7c6)cc5)c5ccc6oc7ccccc7c6c5)cc4)cc23)cc1 ; c1ccc(-c2cccc3c2oc2ccc(-c4ccc(N(c5ccc(-c6cccc7oc8ccccc8c67)cc5)c5ccc6oc7ccccc7c6c5)cc4)cc23)cc1 ; c1ccc(-c2cccc3c2oc2ccc(-c4ccc(N(c5ccc(-c6cccc7c6sc6ccccc67)cc5)c5ccc6oc7ccccc7c6c5)cc4)cc23)cc1 ; c1ccc(-c2cccc3c2oc2ccc(-c4ccc(N(c5ccc(-c6ccc7c(c6)sc6ccccc67)cc5)c5ccc6oc7ccccc7c6c5)cc4)cc23)cc1 ; c1ccc(-c2cccc3c2oc2ccc(-c4ccc(N(c5ccc(-c6ccc7sc8ccccc8c7c6)cc5)c5ccc6oc7ccccc7c6c5)cc4)cc23)cc1]

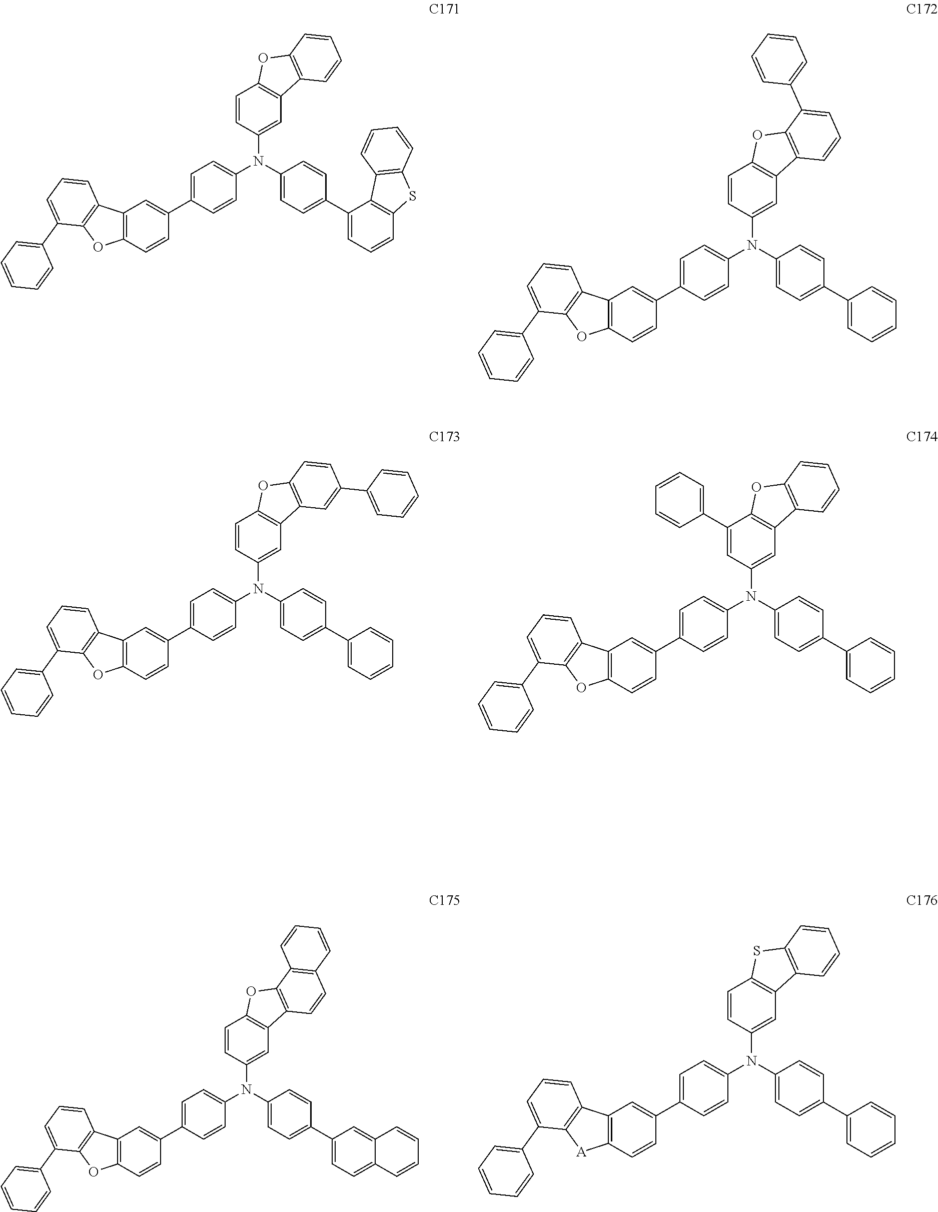

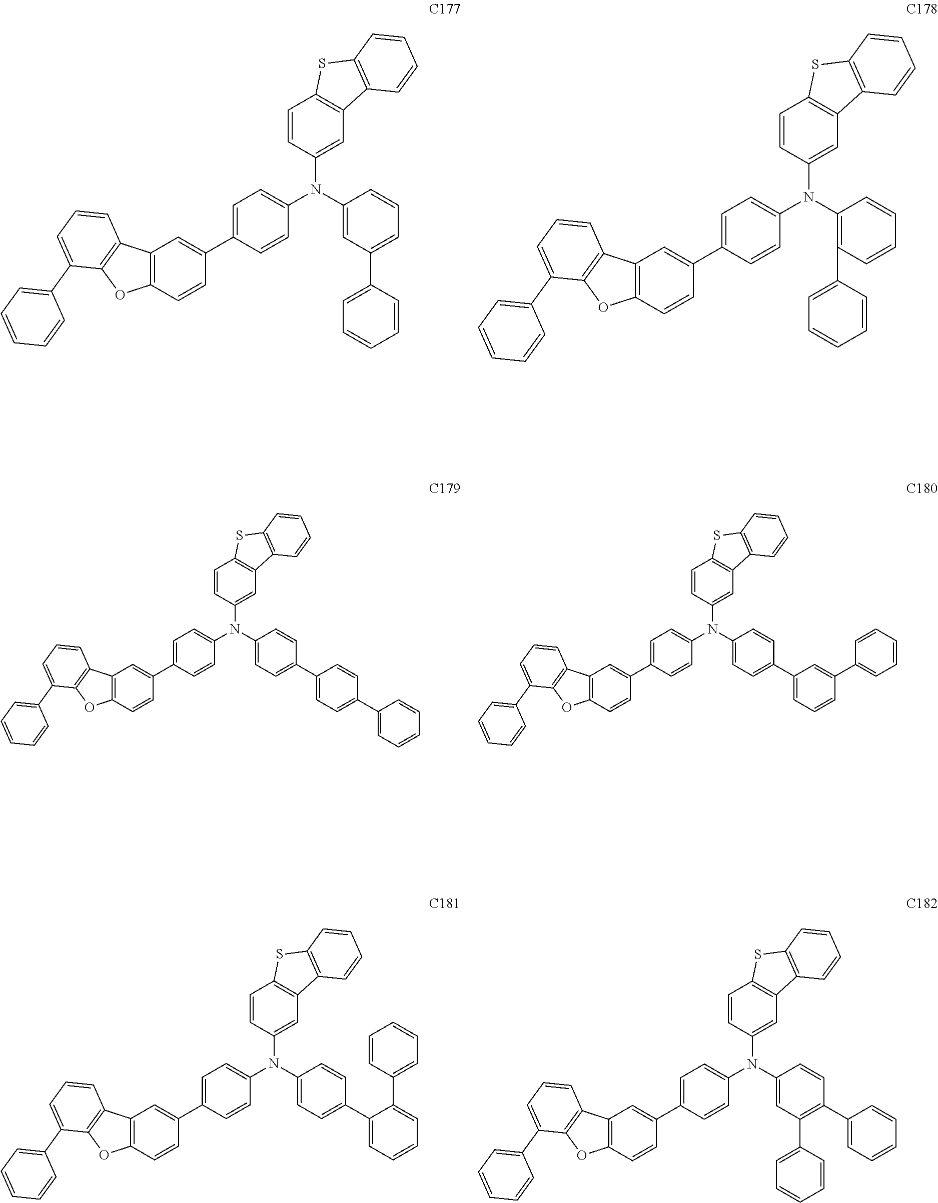

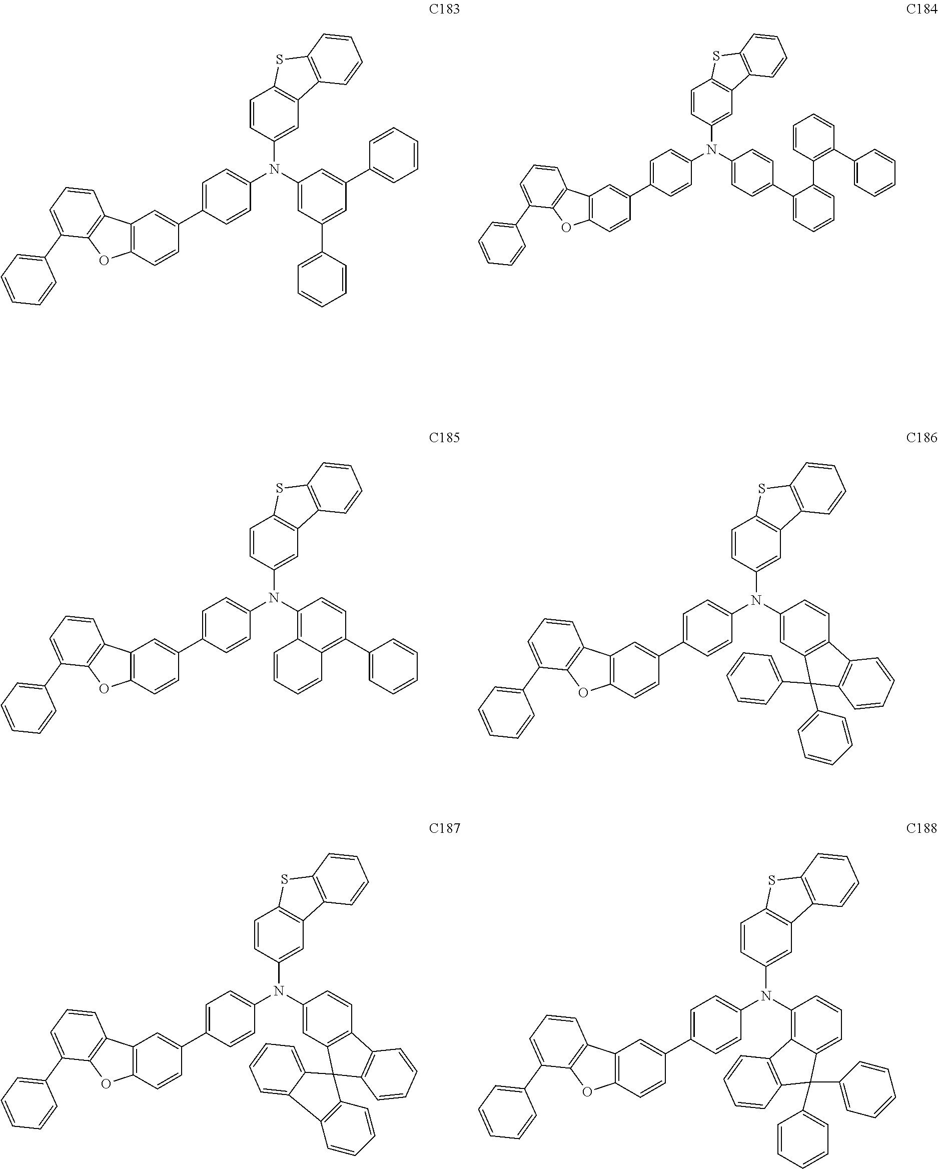

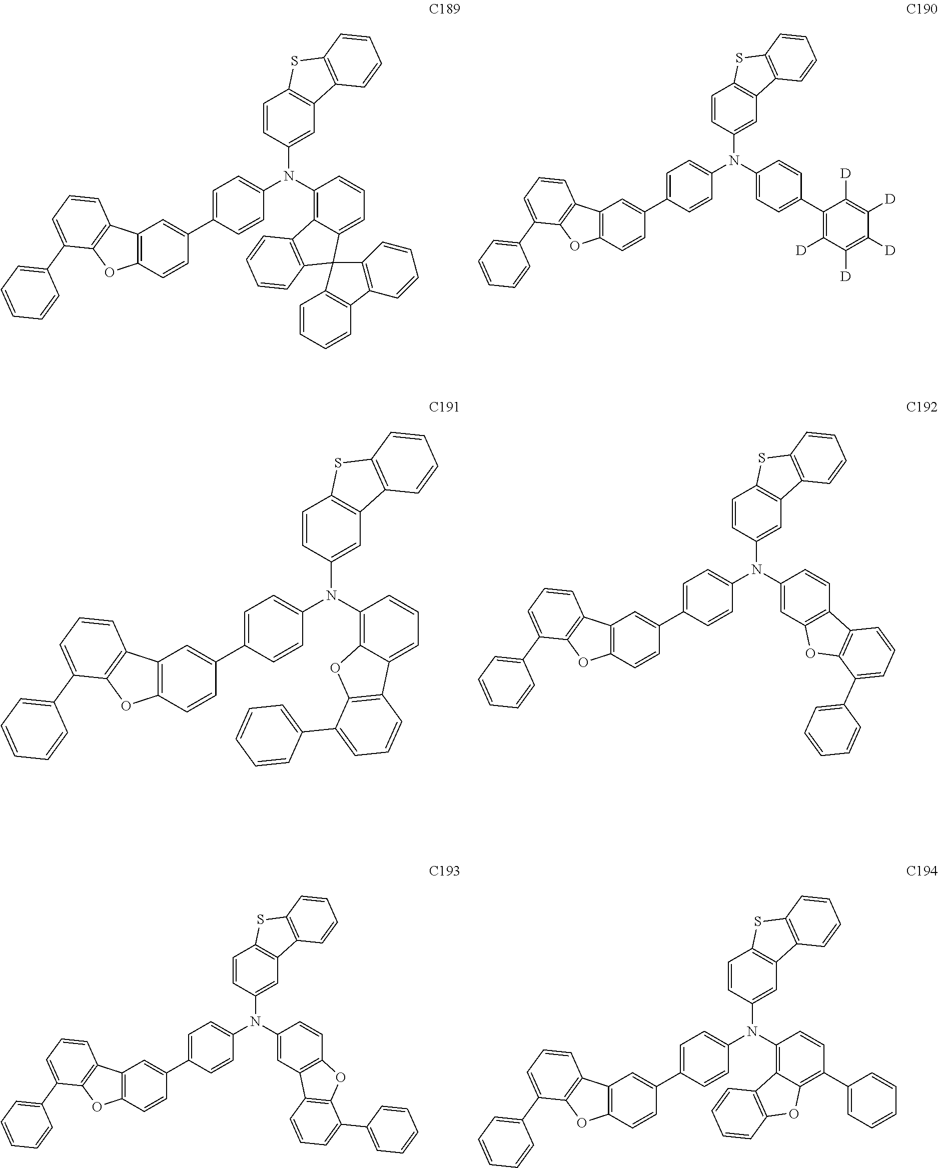

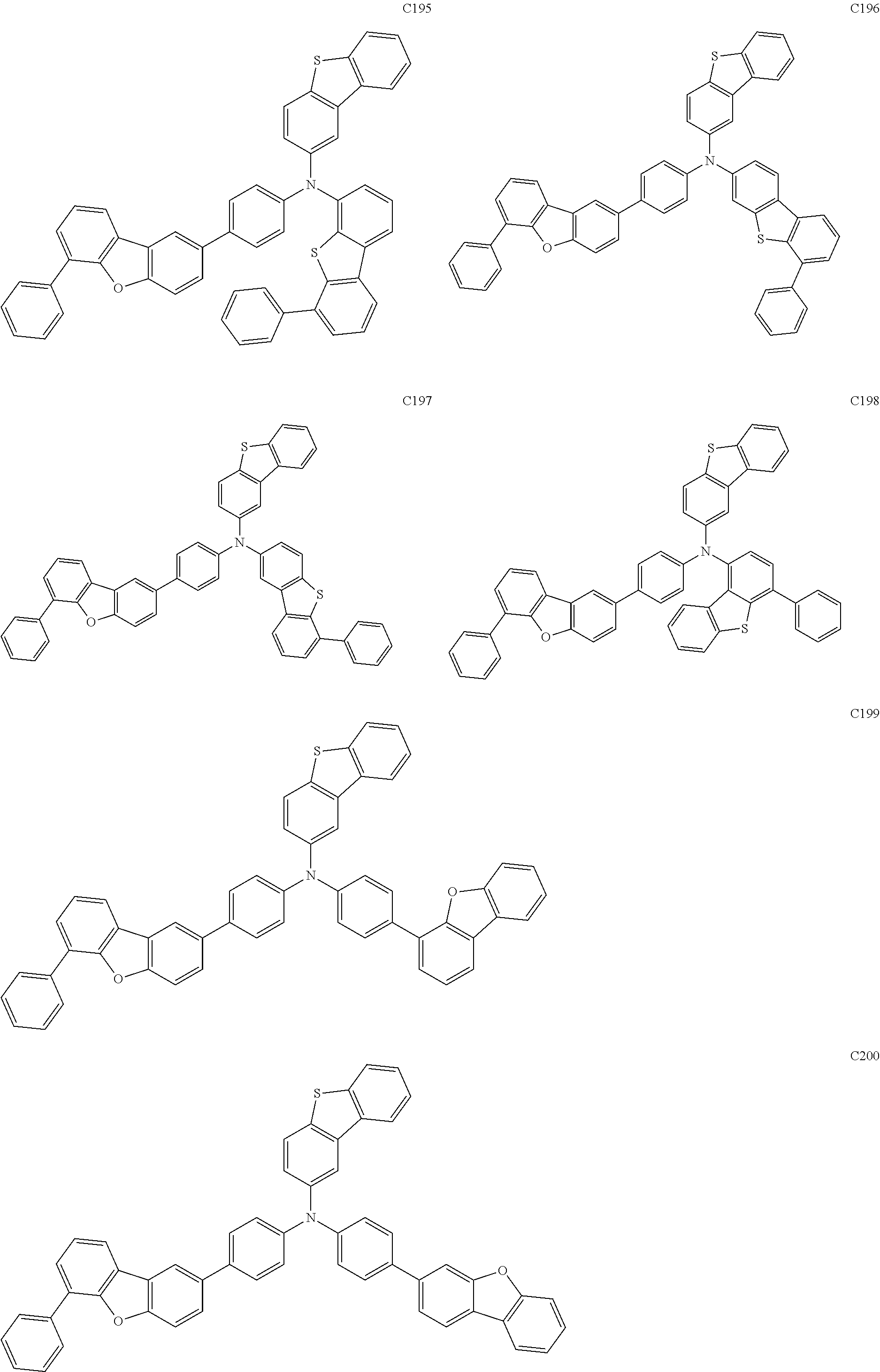

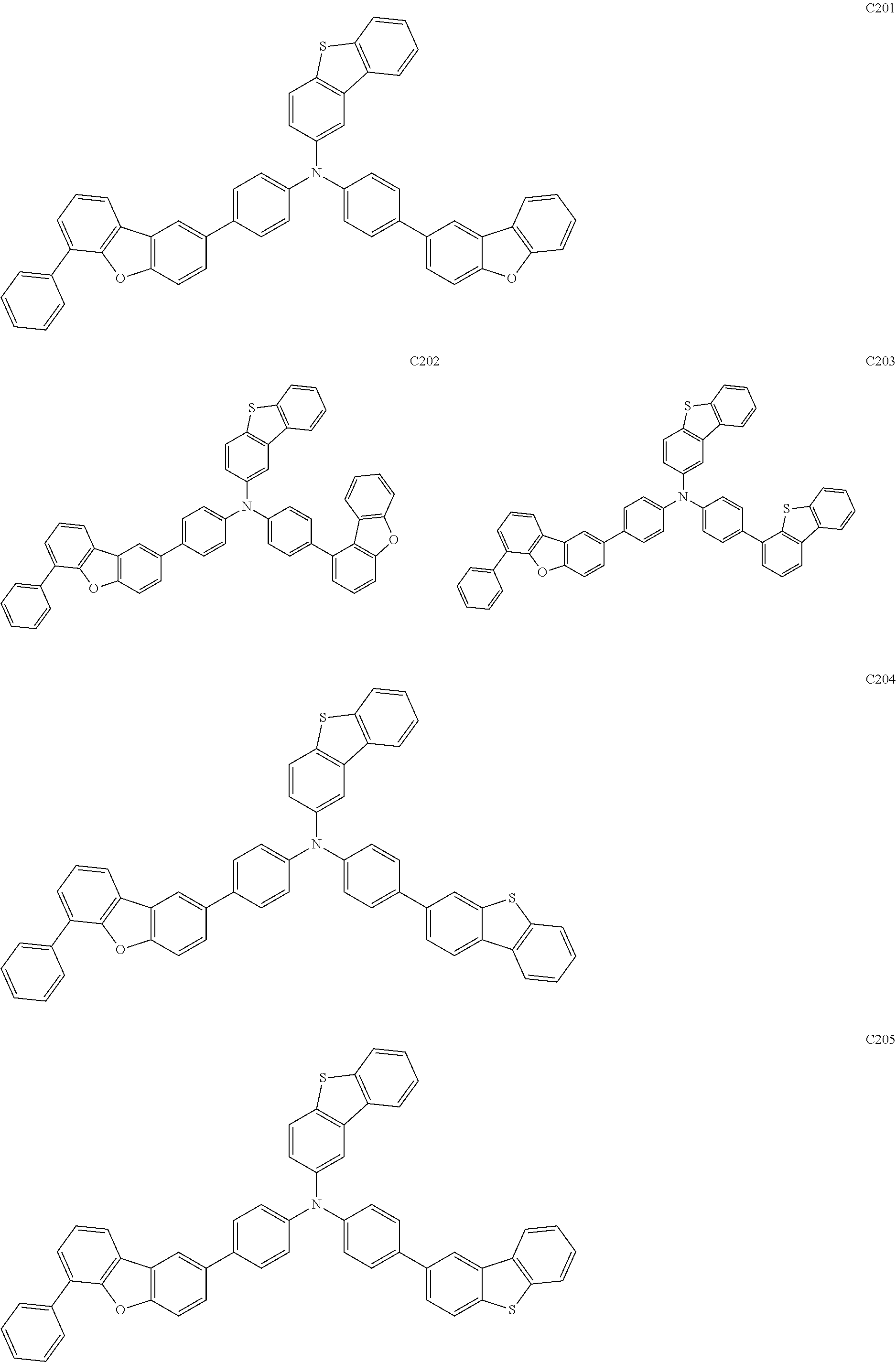 [SMILES: c1ccc(-c2cccc3c2oc2ccc(-c4ccc(N(c5ccc(-c6ccc7oc8ccccc8c7c6)cc5)c5ccc6sc7ccccc7c6c5)cc4)cc23)cc1 ; c1ccc(-c2cccc3c2oc2ccc(-c4ccc(N(c5ccc(-c6cccc7oc8ccccc8c67)cc5)c5ccc6sc7ccccc7c6c5)cc4)cc23)cc1 ; c1ccc(-c2cccc3c2oc2ccc(-c4ccc(N(c5ccc(-c6cccc7c6sc6ccccc67)cc5)c5ccc6sc7ccccc7c6c5)cc4)cc23)cc1 ; c1ccc(-c2cccc3c2oc2ccc(-c4ccc(N(c5ccc(-c6ccc7c(c6)sc6ccccc67)cc5)c5ccc6sc7ccccc7c6c5)cc4)cc23)cc1 ; c1ccc(-c2cccc3c2oc2ccc(-c4ccc(N(c5ccc(-c6ccc7sc8ccccc8c7c6)cc5)c5ccc6sc7ccccc7c6c5)cc4)cc23)cc1]

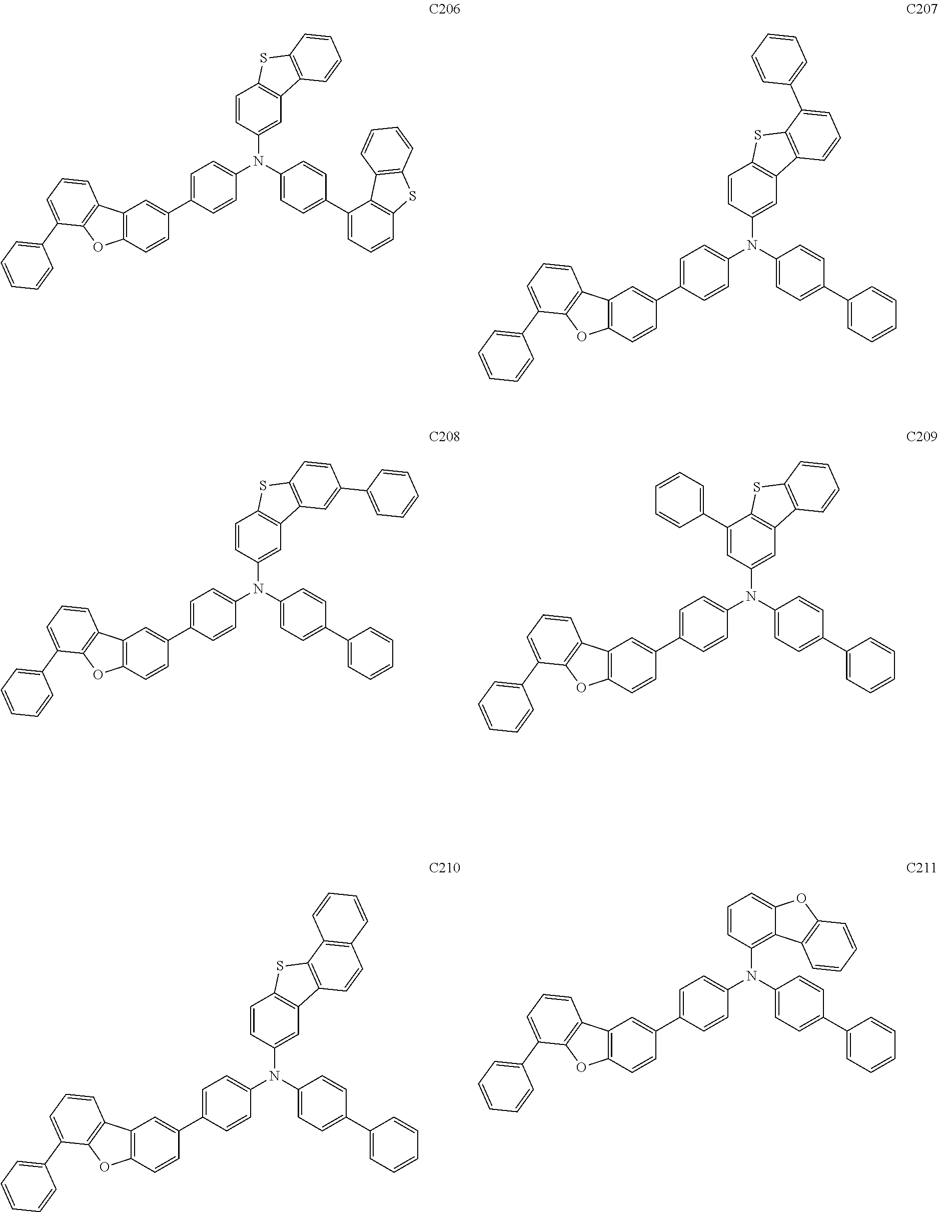

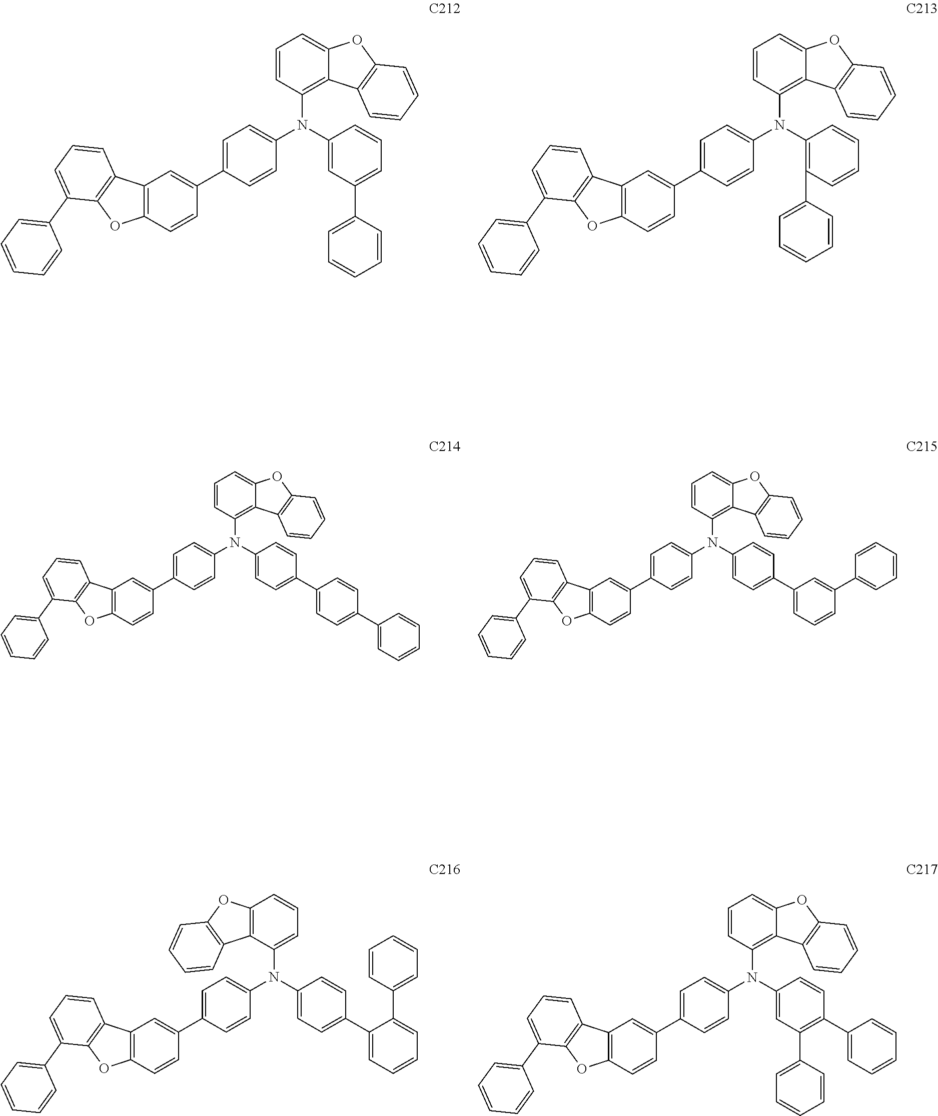

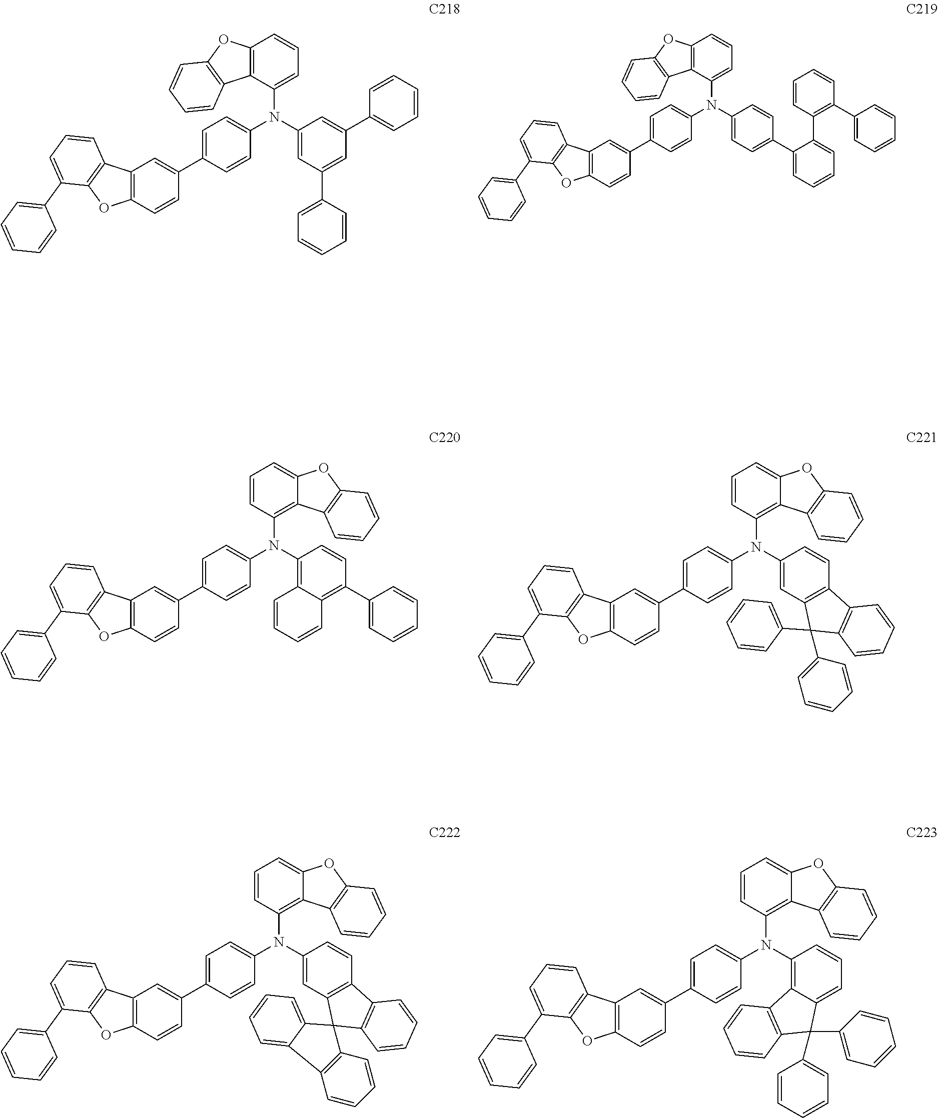

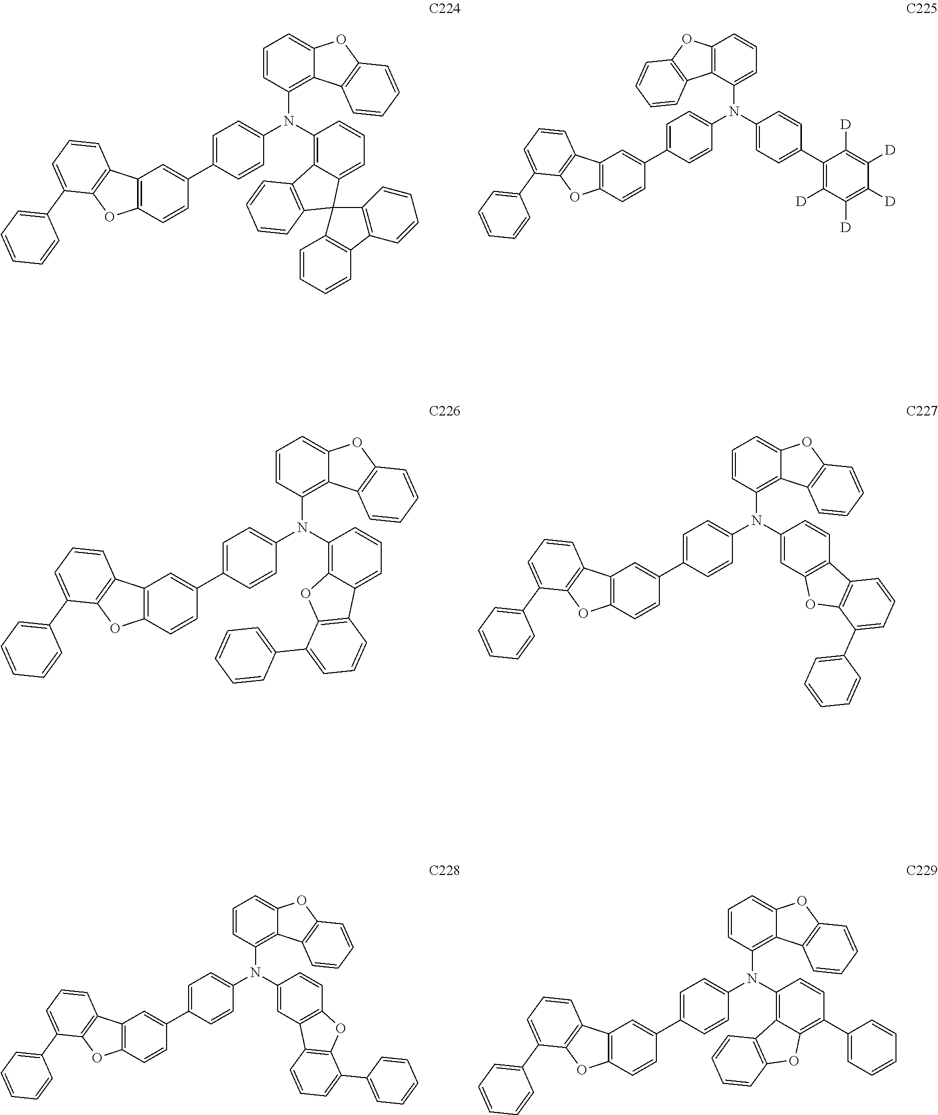

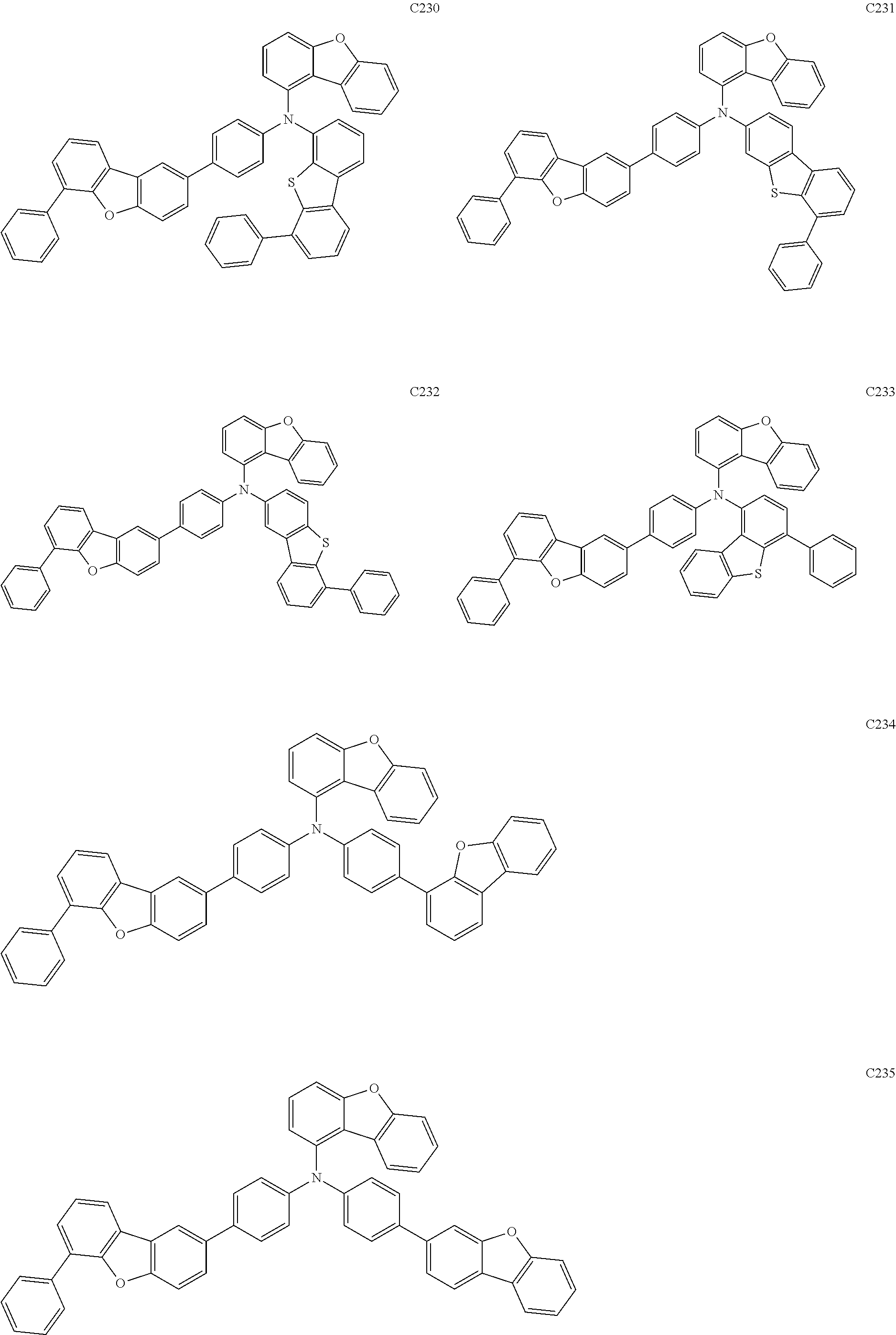

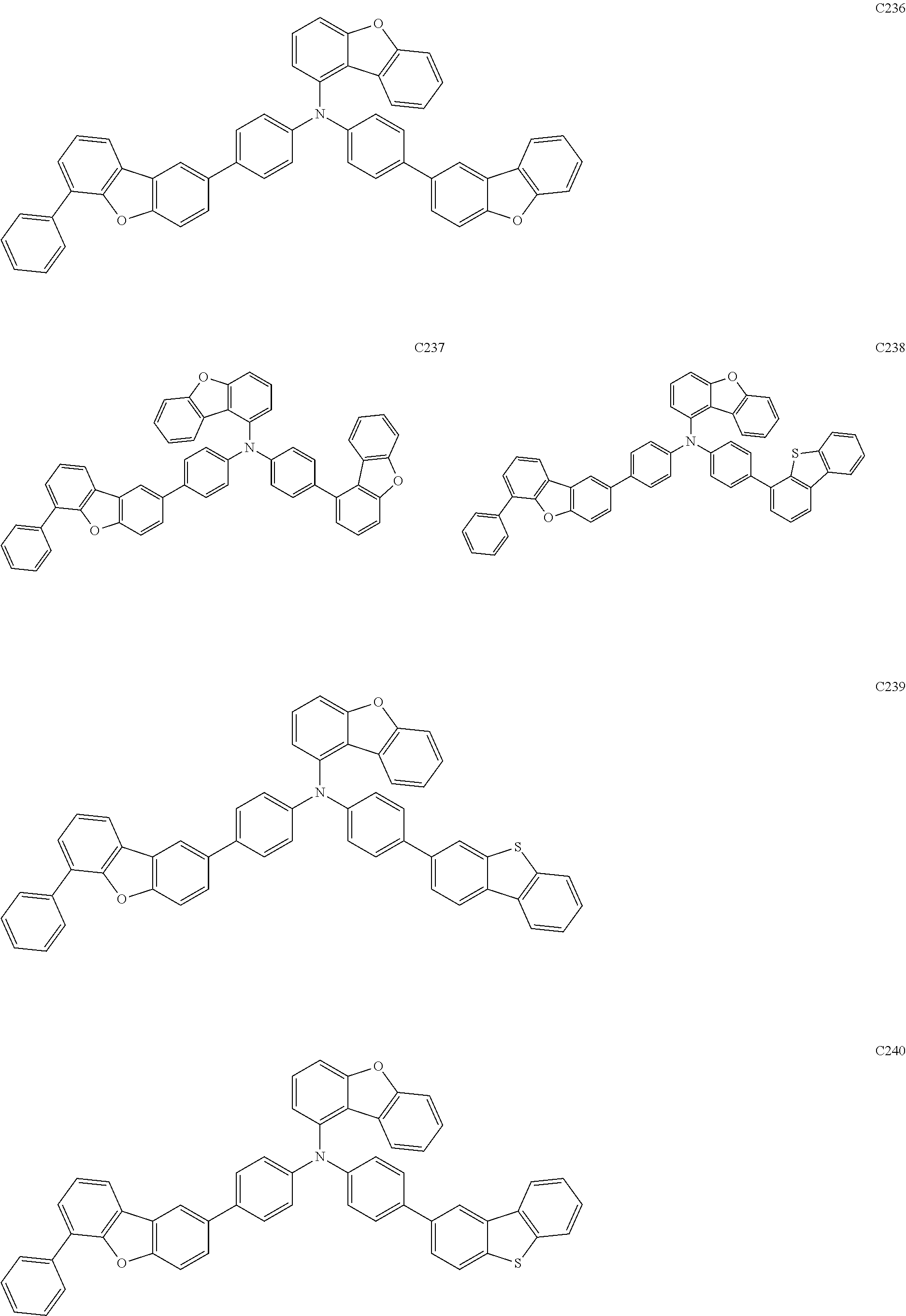 [SMILES: c1ccc(-c2cccc3c2oc2ccc(-c4ccc(N(c5ccc(-c6ccc7oc8ccccc8c7c6)cc5)c5cccc6oc7ccccc7c56)cc4)cc23)cc1 ; c1ccc(-c2cccc3c2oc2ccc(-c4ccc(N(c5ccc(-c6cccc7oc8ccccc8c67)cc5)c5cccc6oc7ccccc7c56)cc4)cc23)cc1 ; c1ccc(-c2cccc3c2oc2ccc(-c4ccc(N(c5ccc(-c6cccc7c6sc6ccccc67)cc5)c5cccc6oc7ccccc7c56)cc4)cc23)cc1 ; c1ccc(-c2cccc3c2oc2ccc(-c4ccc(N(c5ccc(-c6ccc7c(c6)sc6ccccc67)cc5)c5cccc6oc7ccccc7c56)cc4)cc23)cc1 ; c1ccc(-c2cccc3c2oc2ccc(-c4ccc(N(c5ccc(-c6ccc7sc8ccccc8c7c6)cc5)c5cccc6oc7ccccc7c56)cc4)cc23)cc1]

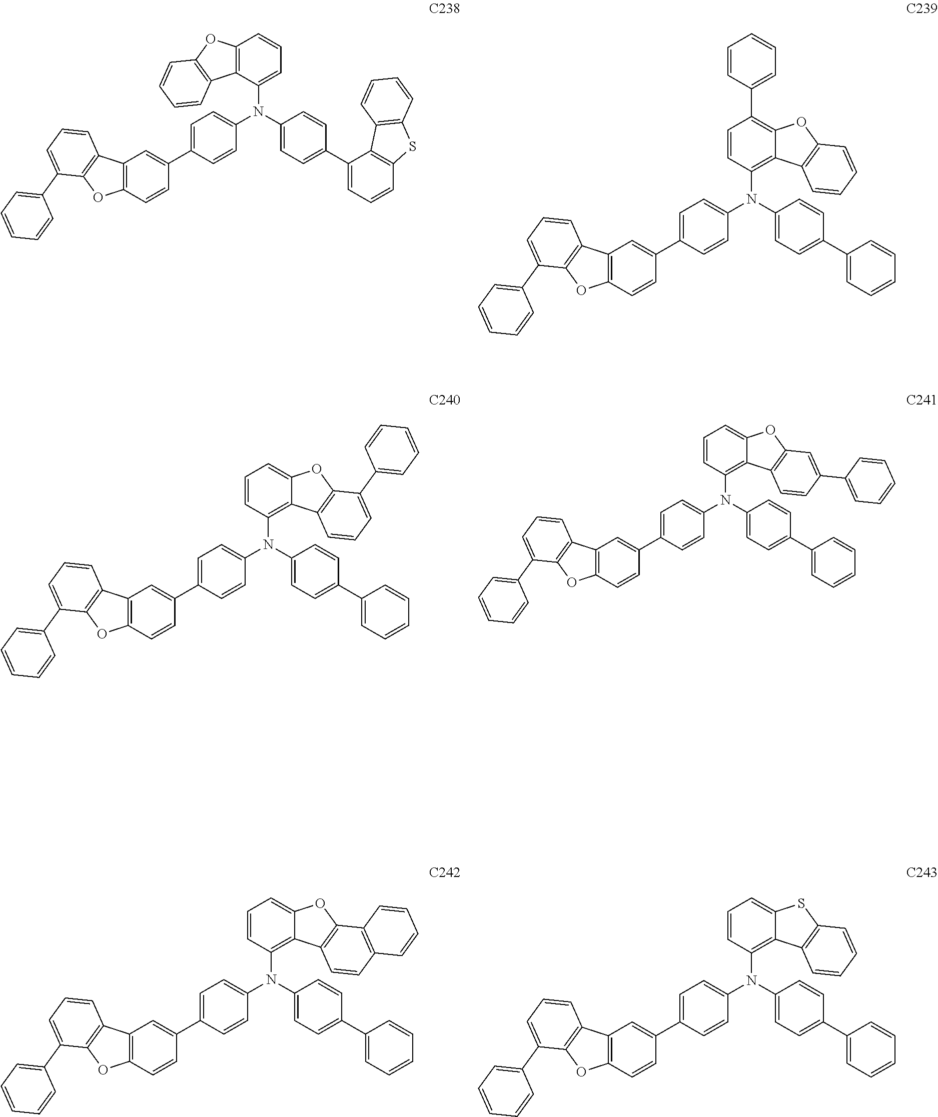

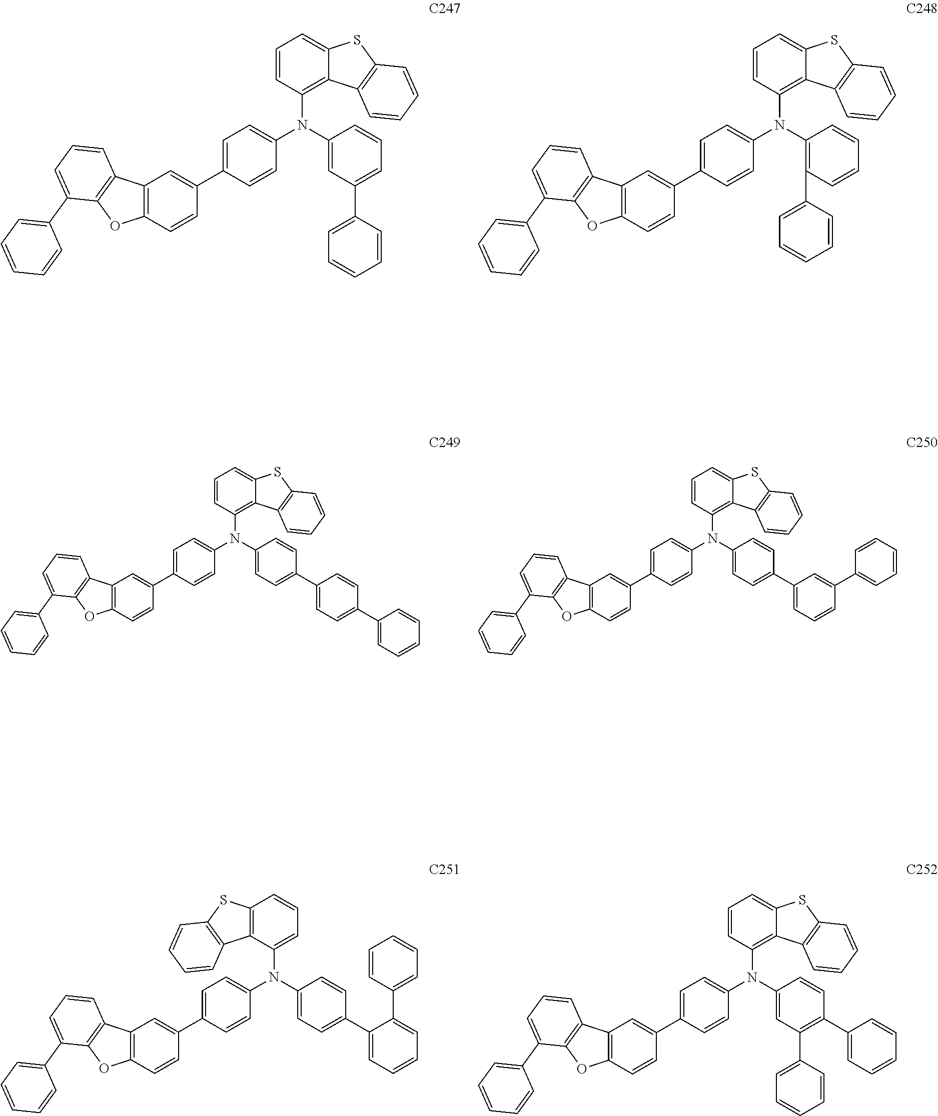

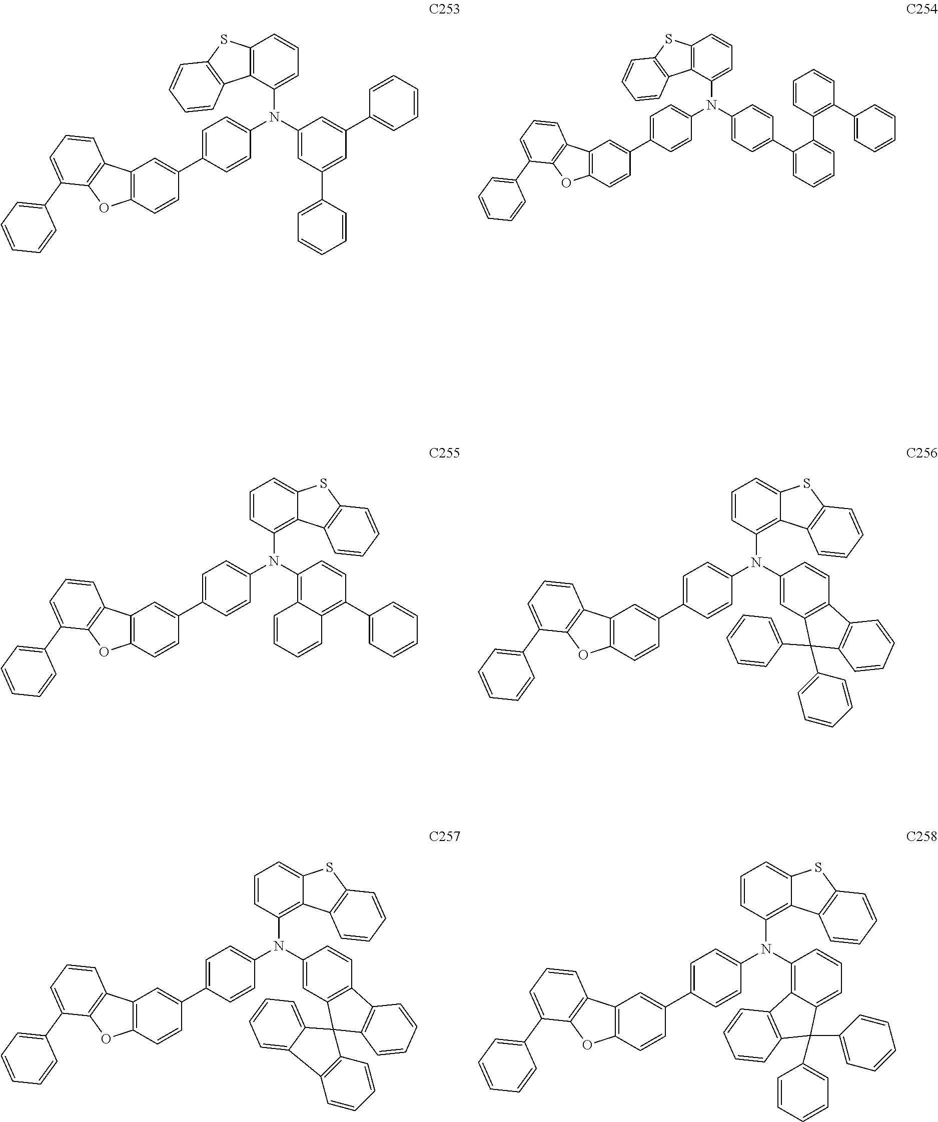

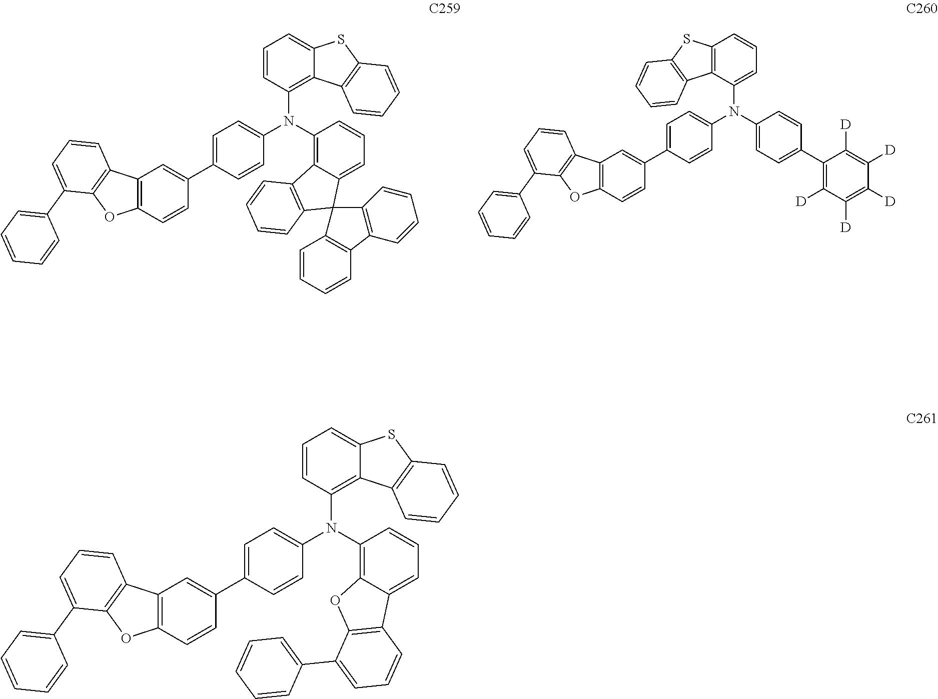 [SMILES: c1ccc(-c2cccc3c2oc2ccc(-c4ccc(N(c5cccc6c5-c5ccccc5C65c6ccccc6-c6ccccc65)c5cccc6sc7ccccc7c56)cc4)cc23)cc1 ; [2H]c1c([2H])c([2H])c(-c2ccc(N(c3ccc(-c4ccc5oc6c(-c7ccccc7)cccc6c5c4)cc3)c3cccc4sc5ccccc5c34)cc2)c([2H])c1[2H] ; c1ccc(-c2cccc3c2oc2ccc(-c4ccc(N(c5cccc6c5oc5c(-c7ccccc7)cccc56)c5cccc6sc7ccccc7c56)cc4)cc23)cc1]

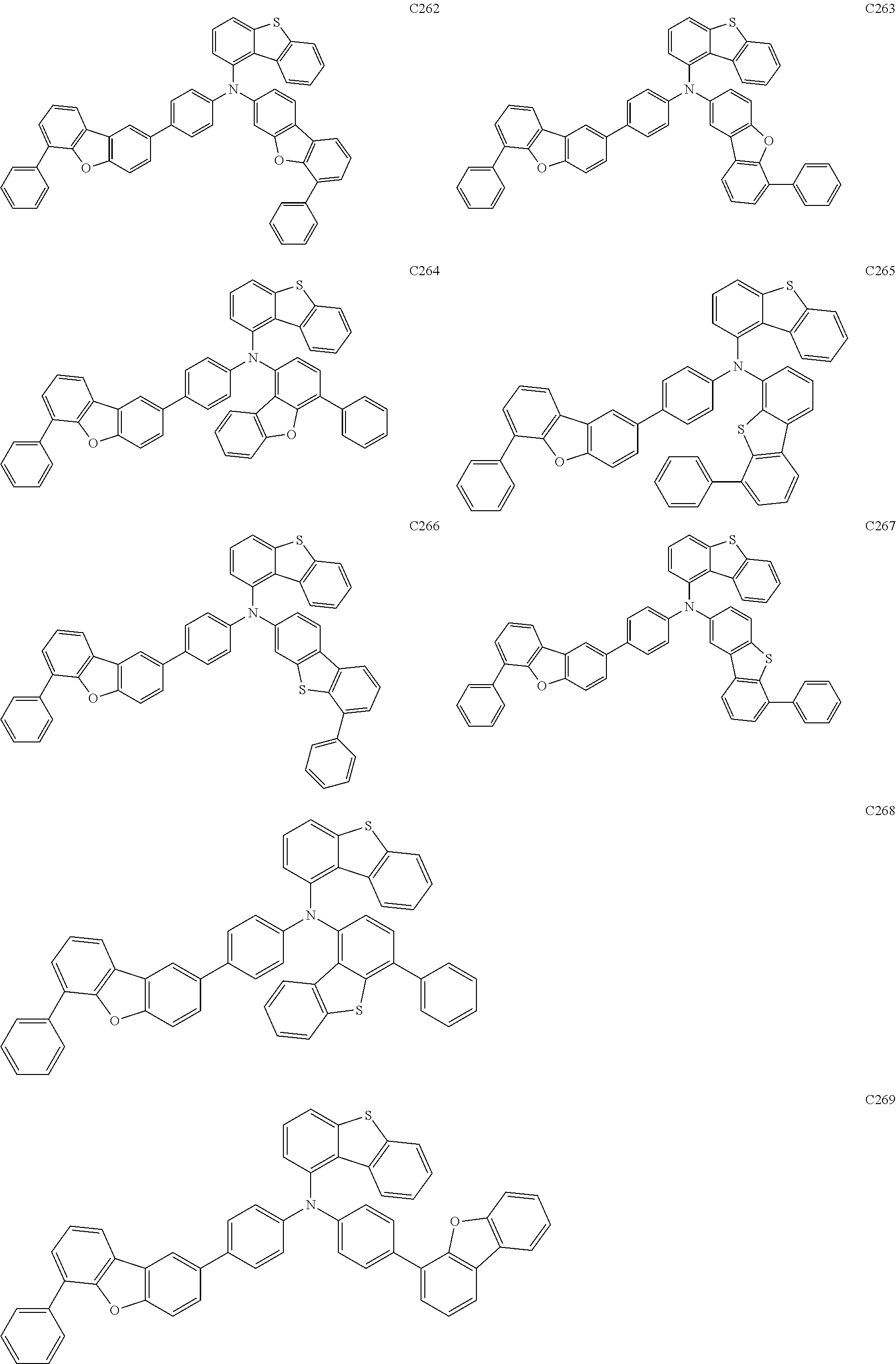

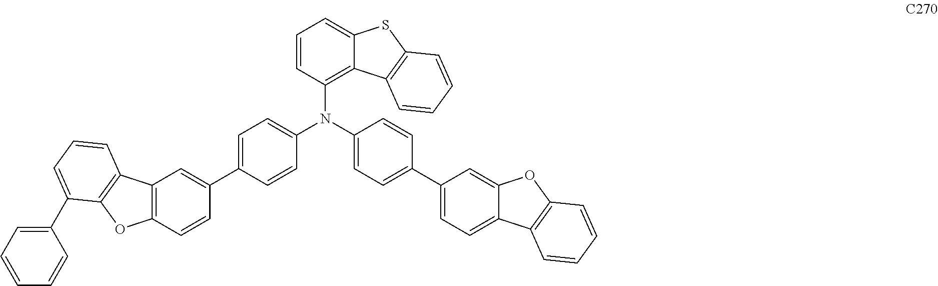 [SMILES: c1ccc(-c2cccc3c2oc2ccc(-c4ccc(N(c5ccc(-c6ccc7c(c6)oc6ccccc67)cc5)c5cccc6sc7ccccc7c56)cc4)cc23)cc1]

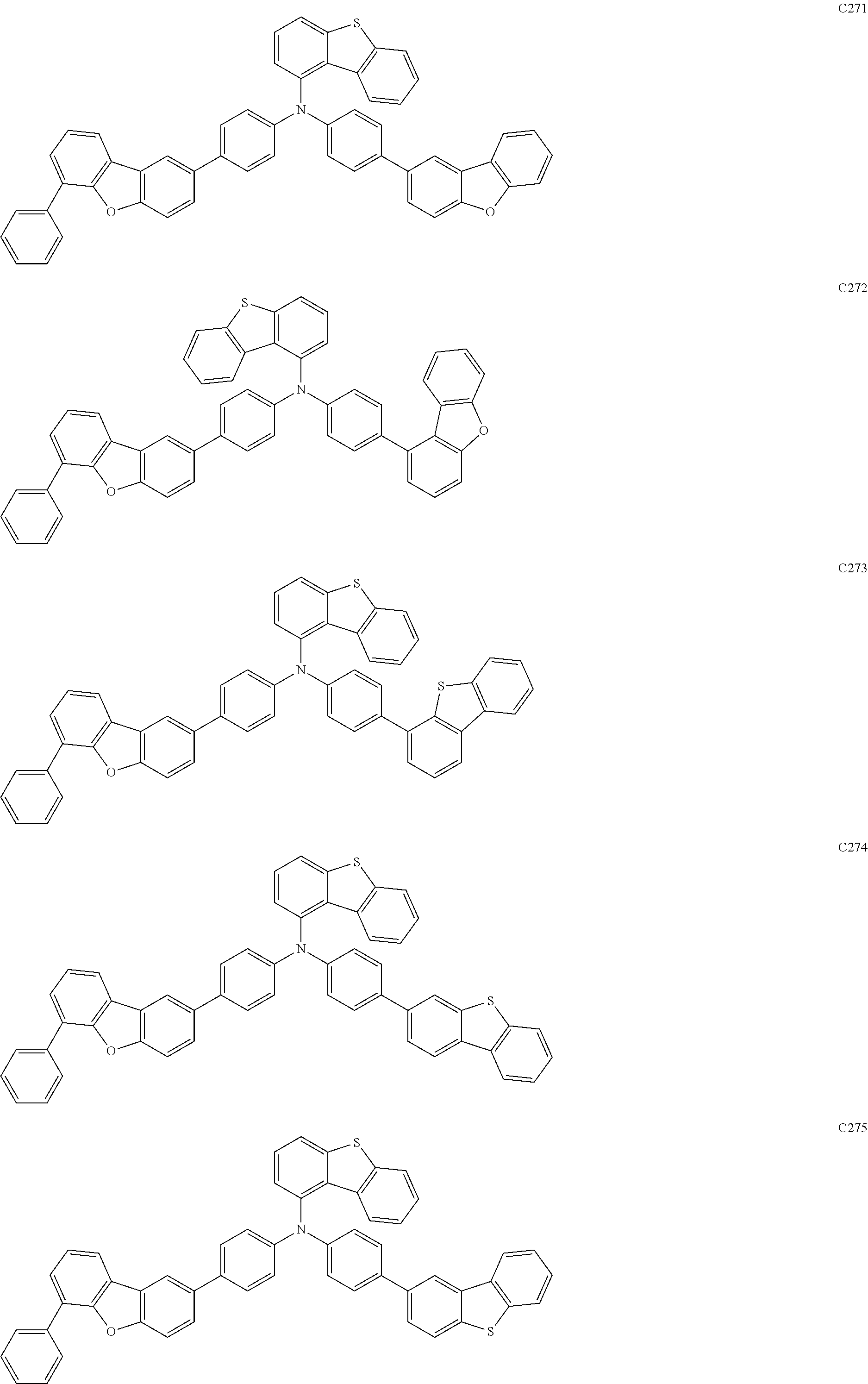 [SMILES: c1ccc(-c2cccc3c2oc2ccc(-c4ccc(N(c5ccc(-c6ccc7oc8ccccc8c7c6)cc5)c5cccc6sc7ccccc7c56)cc4)cc23)cc1 ; c1ccc(-c2cccc3c2oc2ccc(-c4ccc(N(c5ccc(-c6cccc7oc8ccccc8c67)cc5)c5cccc6sc7ccccc7c56)cc4)cc23)cc1 ; c1ccc(-c2cccc3c2oc2ccc(-c4ccc(N(c5ccc(-c6cccc7c6sc6ccccc67)cc5)c5cccc6sc7ccccc7c56)cc4)cc23)cc1 ; c1ccc(-c2cccc3c2oc2ccc(-c4ccc(N(c5ccc(-c6ccc7c(c6)sc6ccccc67)cc5)c5cccc6sc7ccccc7c56)cc4)cc23)cc1 ; c1ccc(-c2cccc3c2oc2ccc(-c4ccc(N(c5ccc(-c6ccc7sc8ccccc8c7c6)cc5)c5cccc6sc7ccccc7c56)cc4)cc23)cc1]

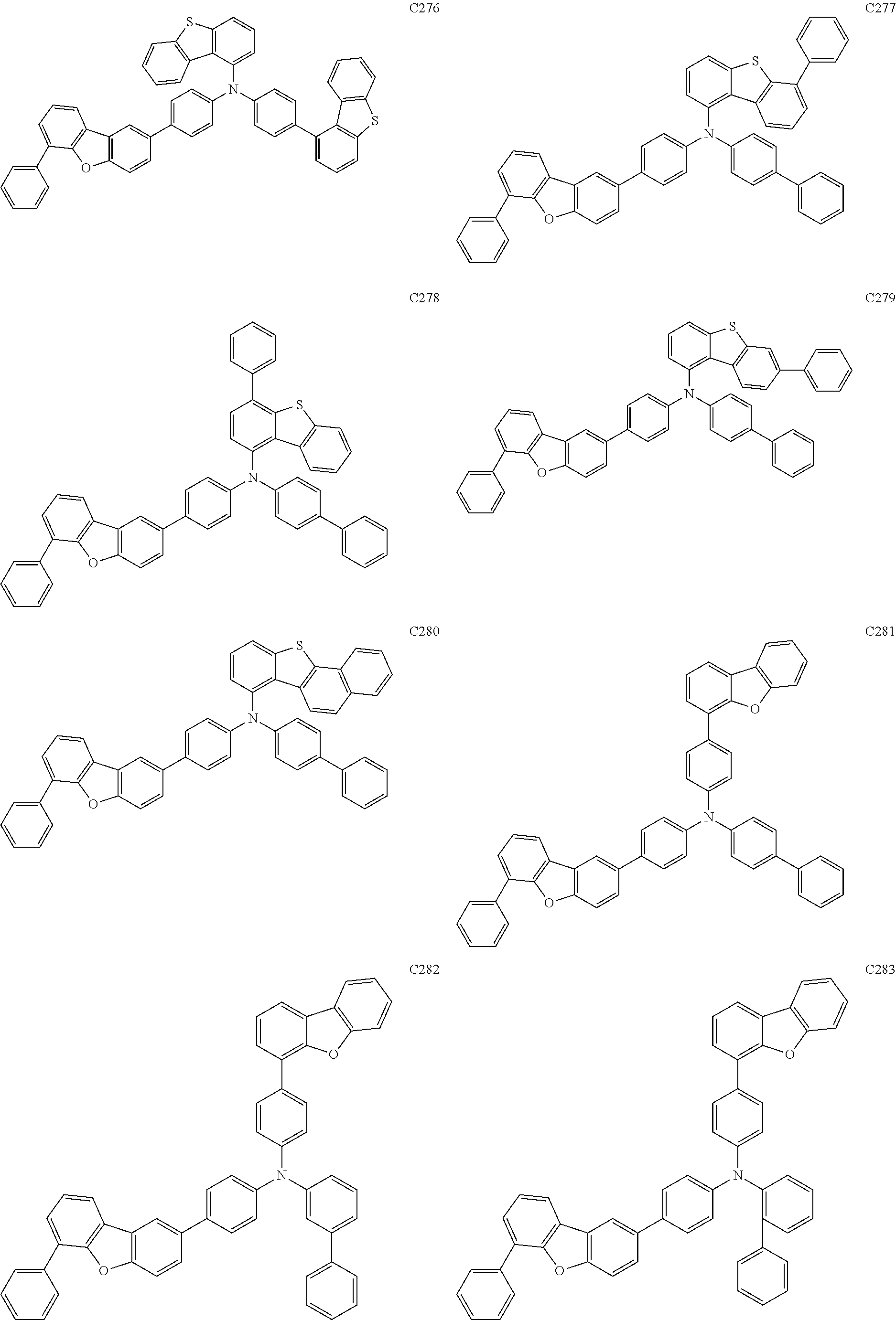

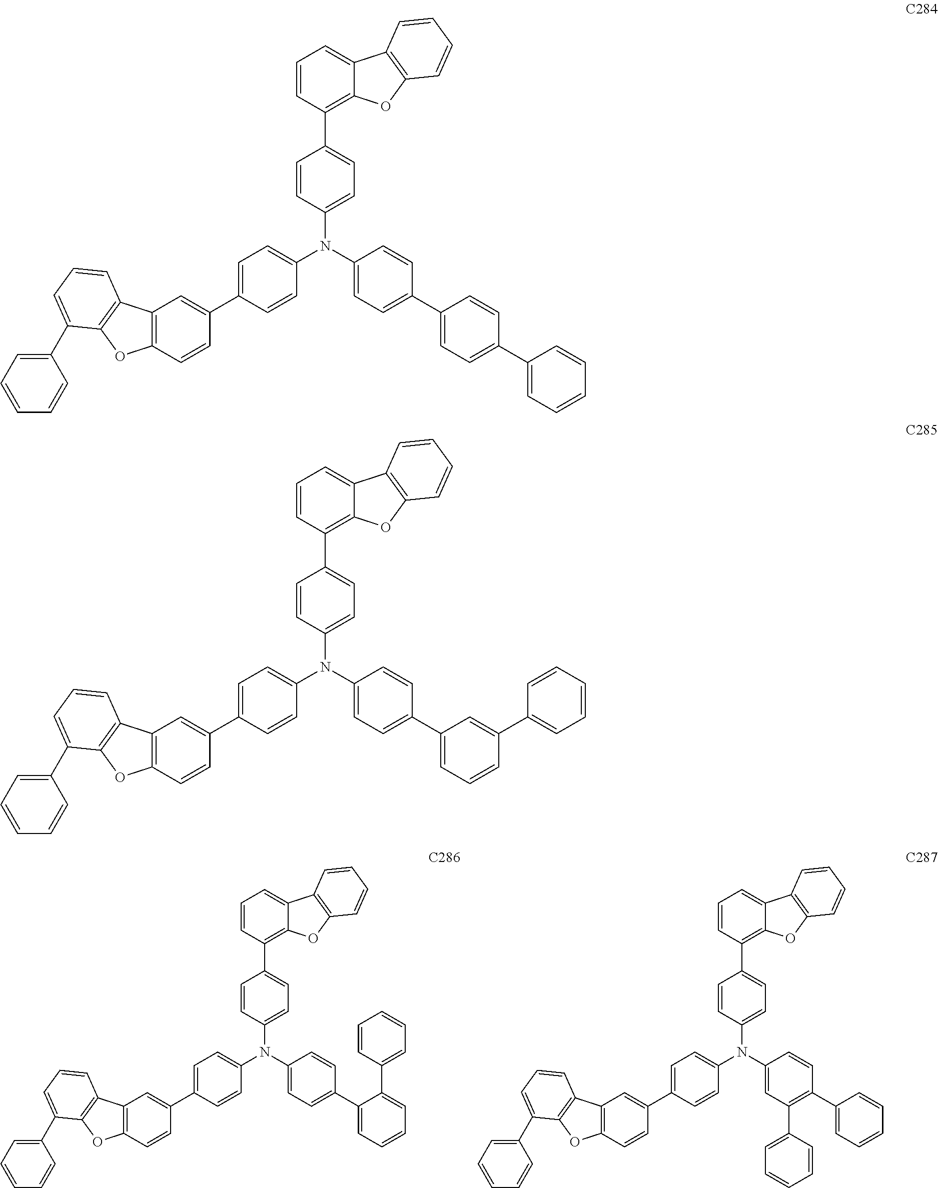 [SMILES: c1ccc(-c2ccc(-c3ccc(N(c4ccc(-c5ccc6oc7c(-c8ccccc8)cccc7c6c5)cc4)c4ccc(-c5cccc6c5oc5ccccc56)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6c5oc5ccccc56)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4cccc5c4oc4ccccc45)cc3)cc2)cc1 ; c1ccc(-c2ccc(N(c3ccc(-c4ccc5oc6c(-c7ccccc7)cccc6c5c4)cc3)c3ccc(-c4cccc5c4oc4ccccc45)cc3)cc2-c2ccccc2)cc1]

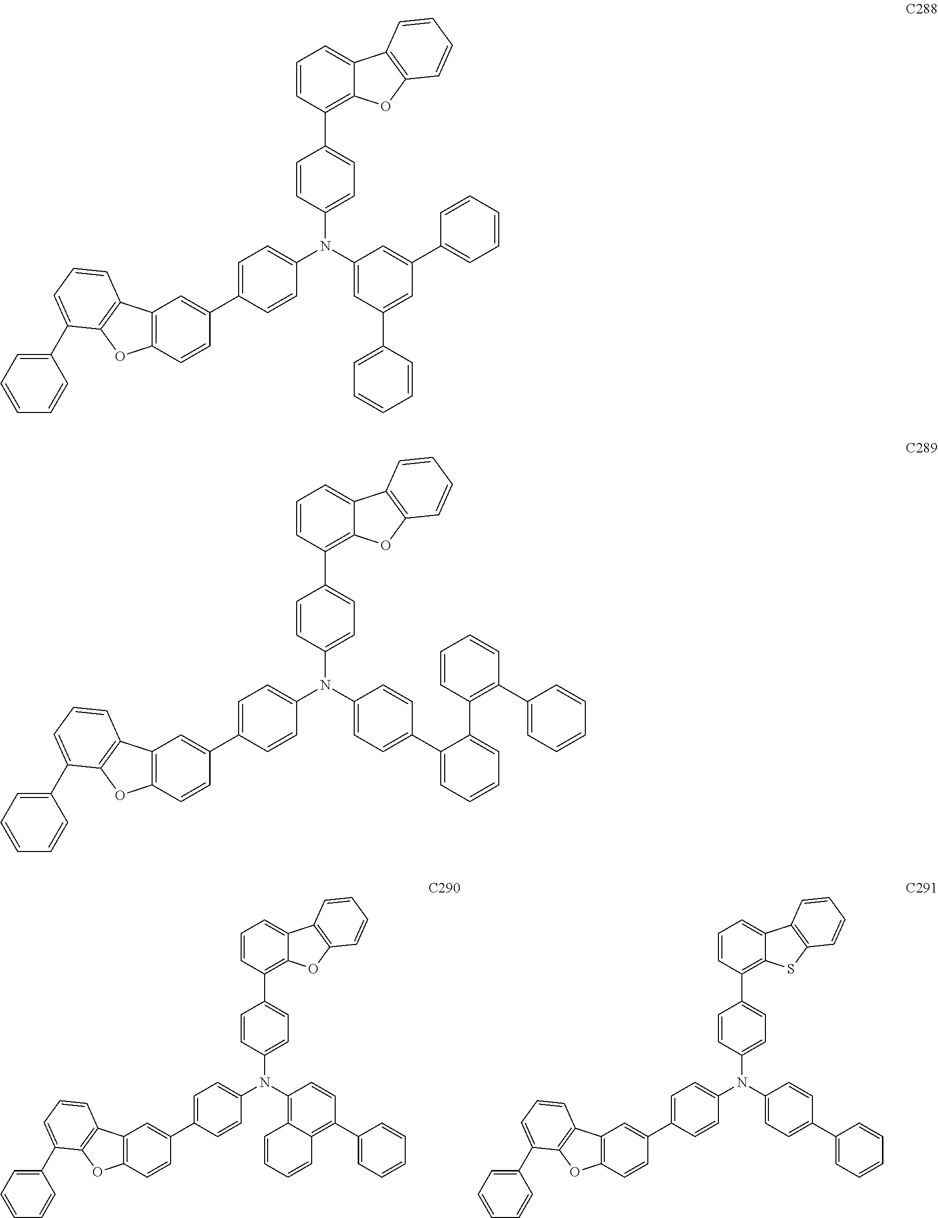 [SMILES: c1ccc(-c2cc(-c3ccccc3)cc(N(c3ccc(-c4ccc5oc6c(-c7ccccc7)cccc6c5c4)cc3)c3ccc(-c4cccc5c4oc4ccccc45)cc3)c2)cc1 ; c1ccc(-c2ccccc2-c2ccccc2-c2ccc(N(c3ccc(-c4ccc5oc6c(-c7ccccc7)cccc6c5c4)cc3)c3ccc(-c4cccc5c4oc4ccccc45)cc3)cc2)cc1 ; c1ccc(-c2ccc(N(c3ccc(-c4ccc5oc6c(-c7ccccc7)cccc6c5c4)cc3)c3ccc(-c4cccc5c4oc4ccccc45)cc3)c3ccccc23)cc1 ; c1ccc(-c2ccc(N(c3ccc(-c4ccc5oc6c(-c7ccccc7)cccc6c5c4)cc3)c3ccc(-c4cccc5c4sc4ccccc45)cc3)cc2)cc1]

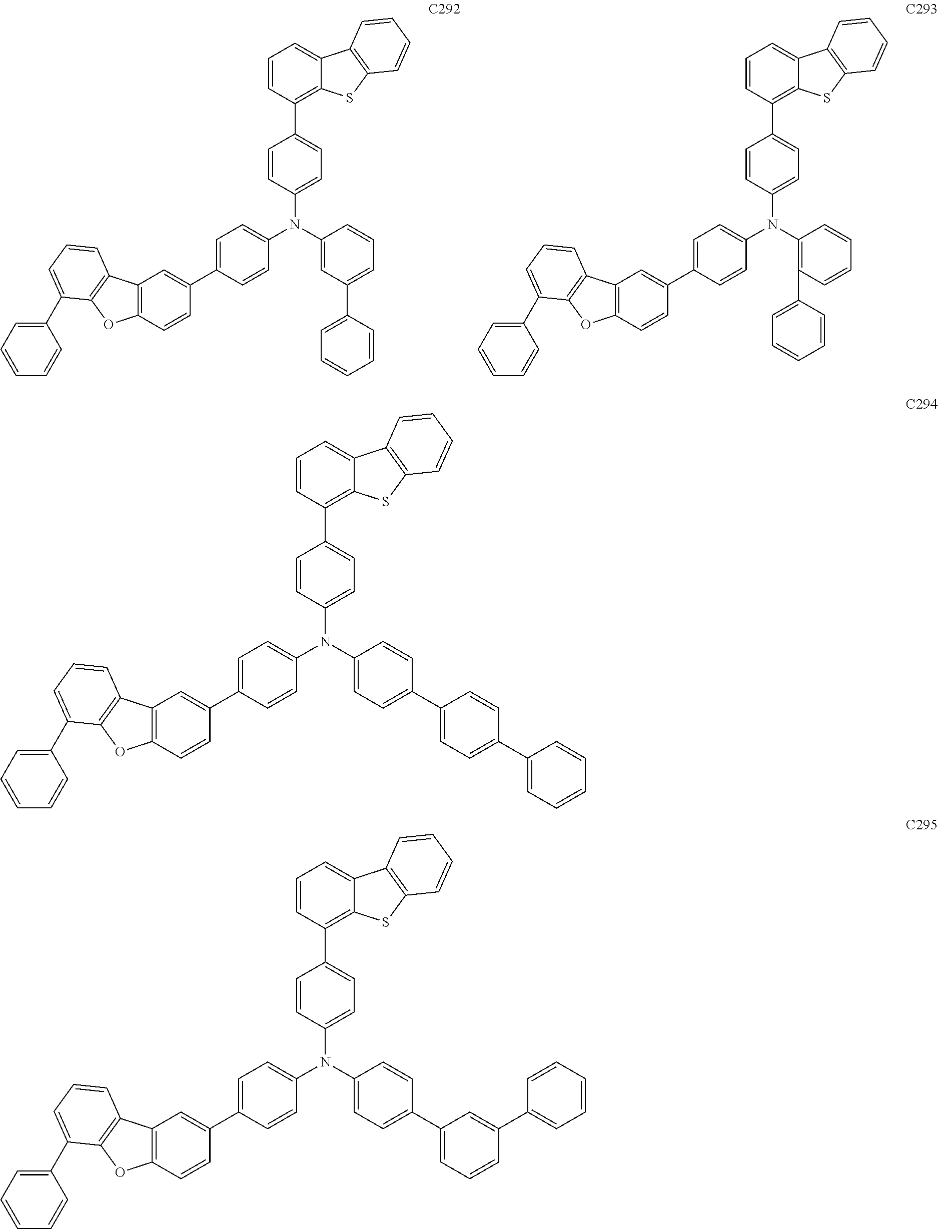 [SMILES: c1ccc(-c2cccc(N(c3ccc(-c4ccc5oc6c(-c7ccccc7)cccc6c5c4)cc3)c3ccc(-c4cccc5c4sc4ccccc45)cc3)c2)cc1 ; c1ccc(-c2ccccc2N(c2ccc(-c3ccc4oc5c(-c6ccccc6)cccc5c4c3)cc2)c2ccc(-c3cccc4c3sc3ccccc34)cc2)cc1 ; c1ccc(-c2ccc(-c3ccc(N(c4ccc(-c5ccc6oc7c(-c8ccccc8)cccc7c6c5)cc4)c4ccc(-c5cccc6c5sc5ccccc56)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6c5sc5ccccc56)cc4)cc3)c2)cc1]

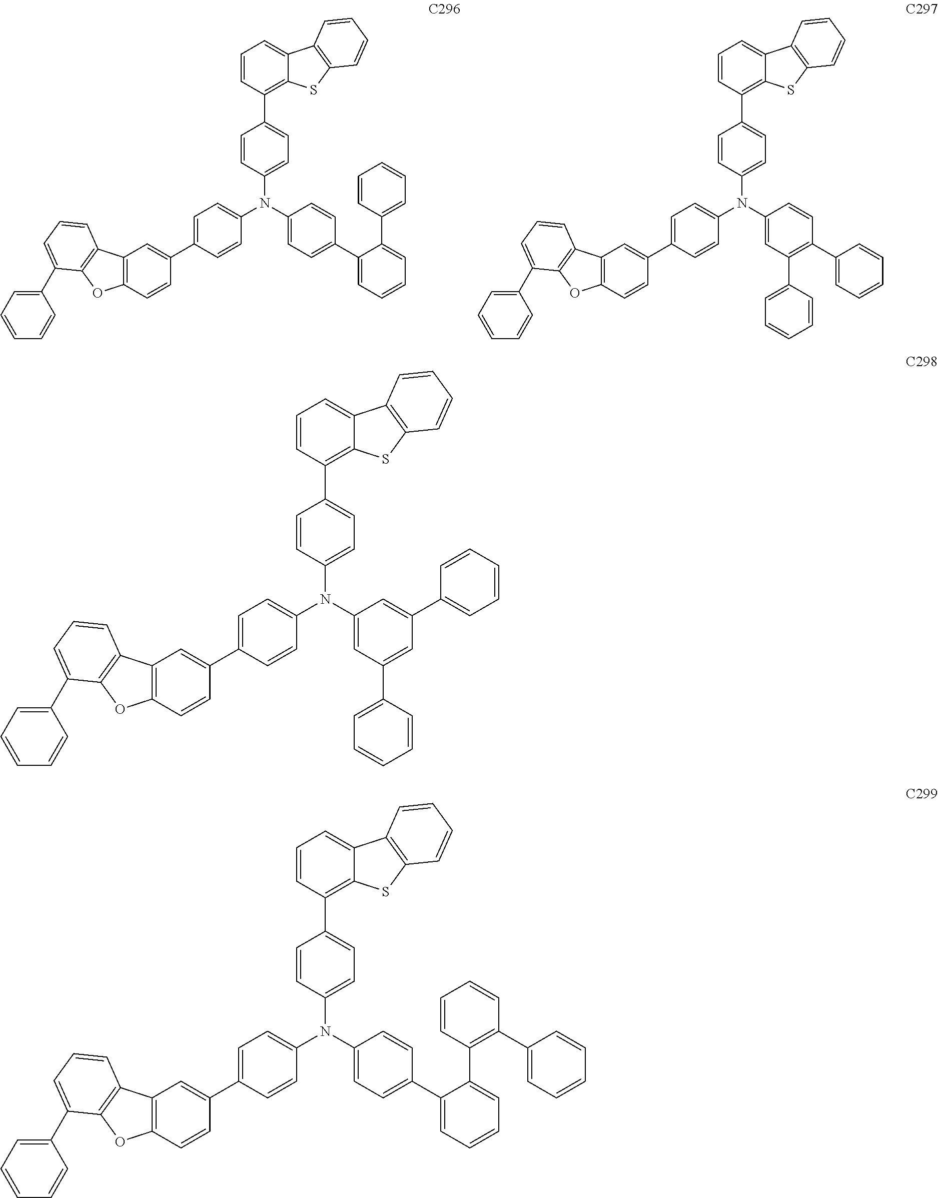

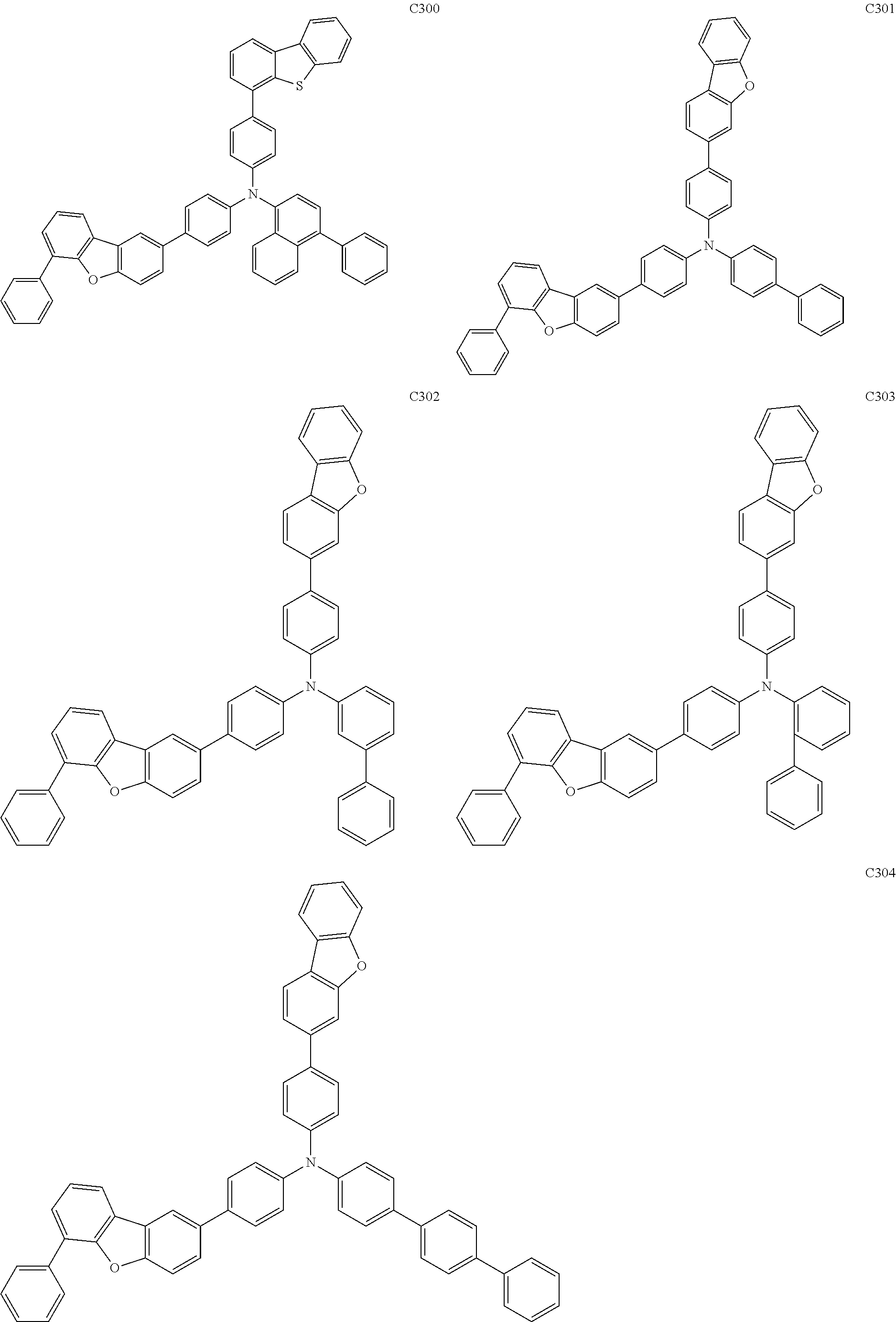

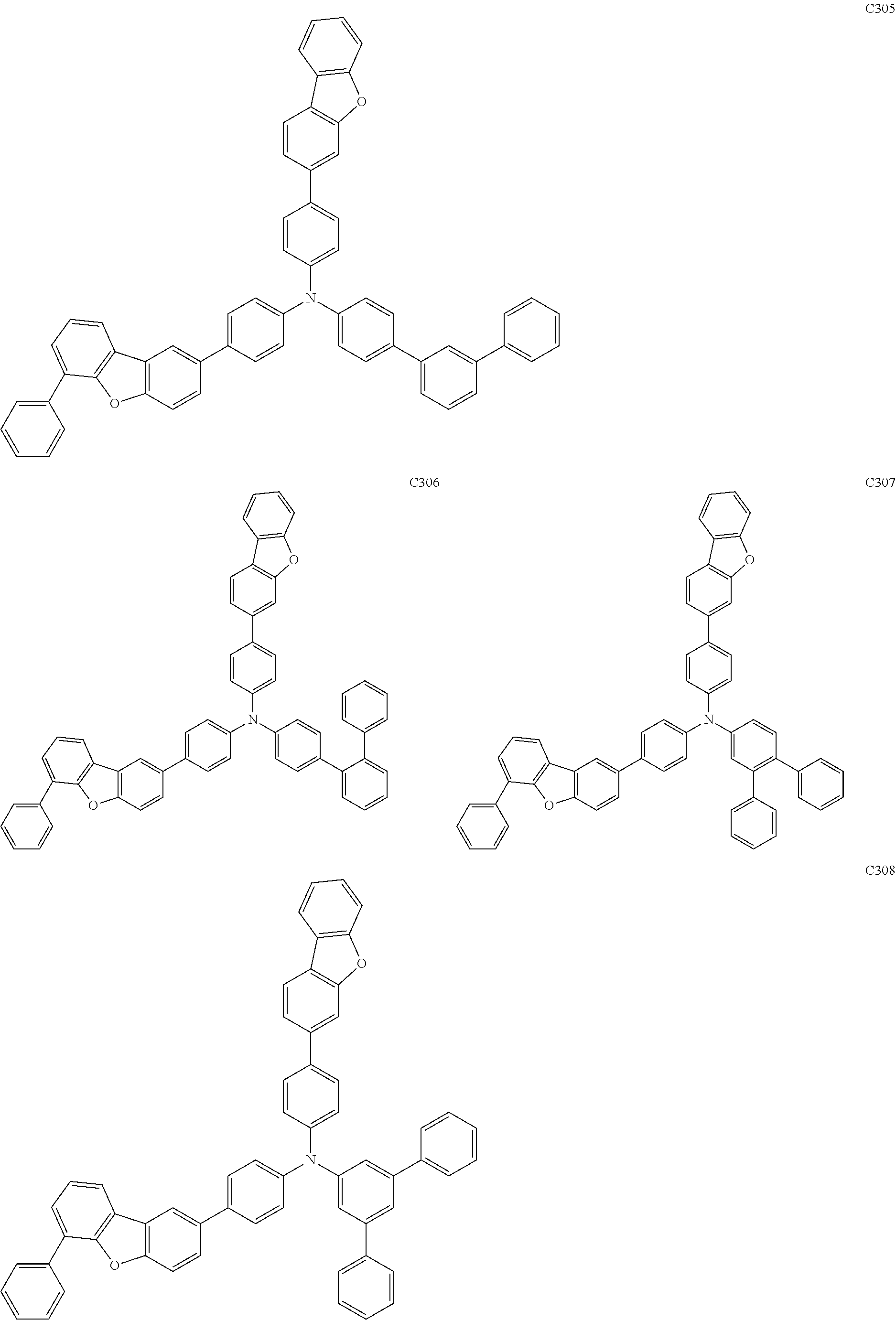 [SMILES: c1ccc(-c2cccc(-c3ccc(N(c4ccc(-c5ccc6c(c5)oc5ccccc56)cc4)c4ccc(-c5ccc6oc7c(-c8ccccc8)cccc7c6c5)cc4)cc3)c2)cc1 ; c1ccc(-c2ccccc2-c2ccc(N(c3ccc(-c4ccc5c(c4)oc4ccccc45)cc3)c3ccc(-c4ccc5oc6c(-c7ccccc7)cccc6c5c4)cc3)cc2)cc1 ; c1ccc(-c2ccc(N(c3ccc(-c4ccc5c(c4)oc4ccccc45)cc3)c3ccc(-c4ccc5oc6c(-c7ccccc7)cccc6c5c4)cc3)cc2-c2ccccc2)cc1 ; c1ccc(-c2cc(-c3ccccc3)cc(N(c3ccc(-c4ccc5c(c4)oc4ccccc45)cc3)c3ccc(-c4ccc5oc6c(-c7ccccc7)cccc6c5c4)cc3)c2)cc1]

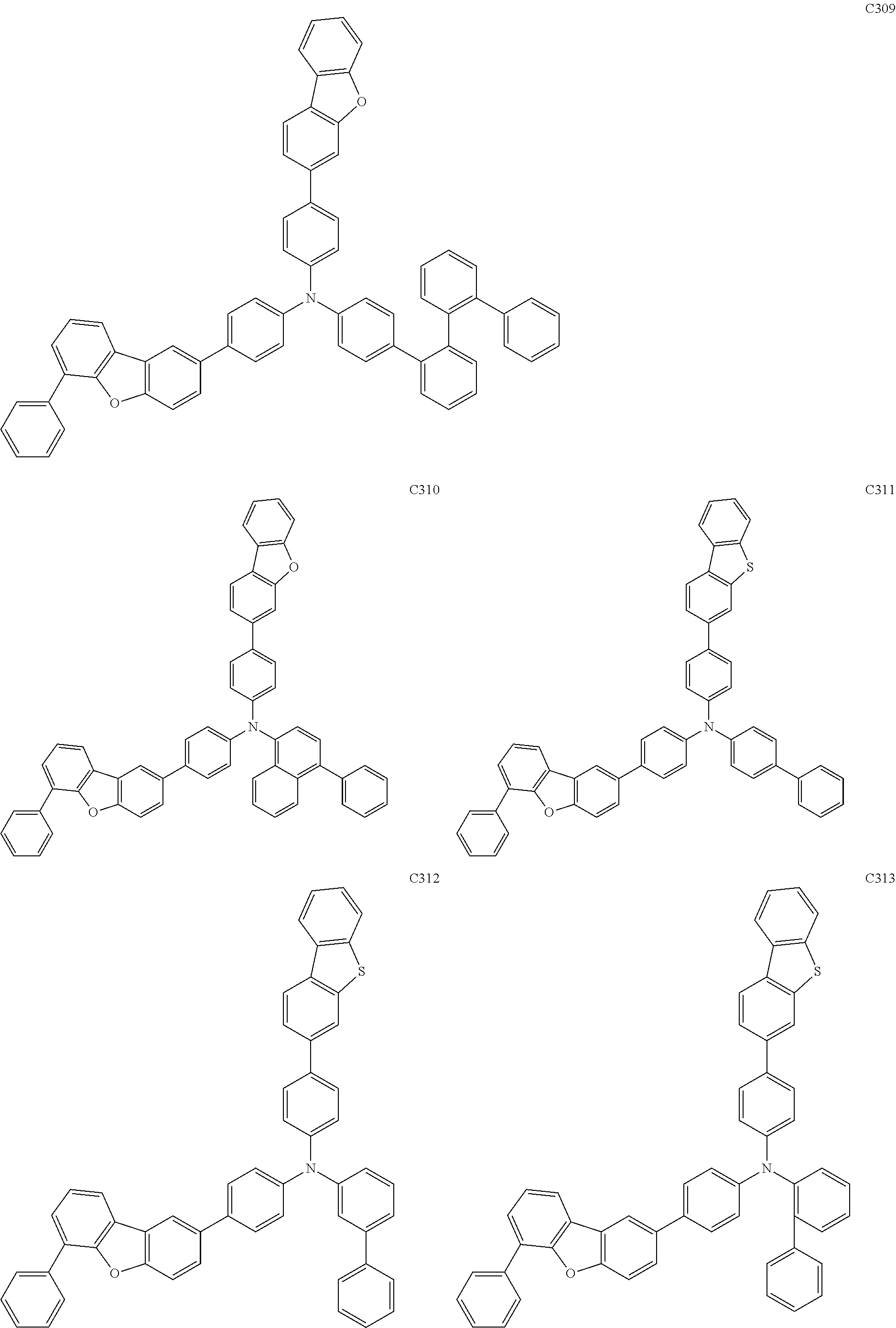

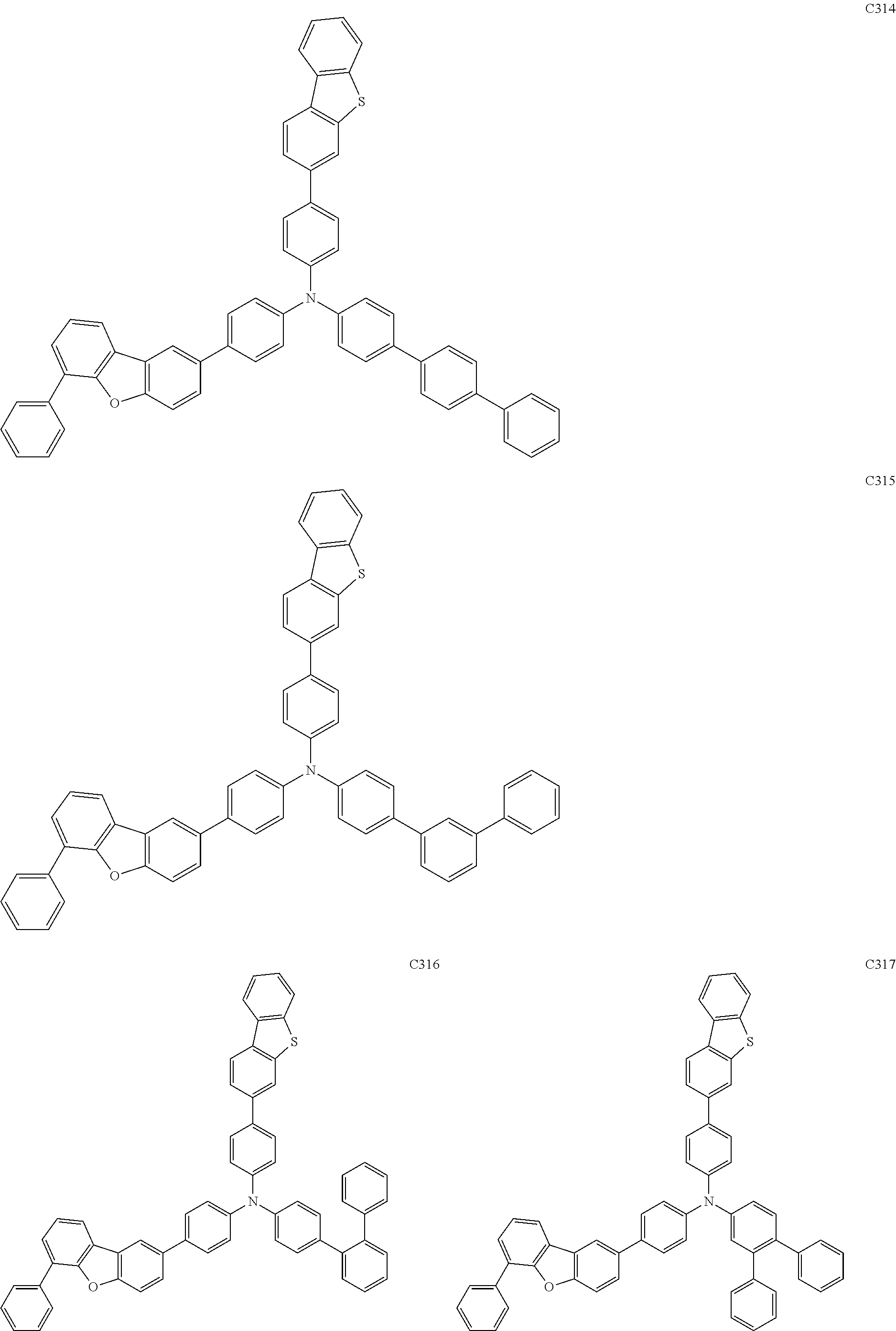 [SMILES: c1ccc(-c2ccc(-c3ccc(N(c4ccc(-c5ccc6c(c5)sc5ccccc56)cc4)c4ccc(-c5ccc6oc7c(-c8ccccc8)cccc7c6c5)cc4)cc3)cc2)cc1 ; c1ccc(-c2cccc(-c3ccc(N(c4ccc(-c5ccc6c(c5)sc5ccccc56)cc4)c4ccc(-c5ccc6oc7c(-c8ccccc8)cccc7c6c5)cc4)cc3)c2)cc1 ; c1ccc(-c2ccccc2-c2ccc(N(c3ccc(-c4ccc5c(c4)sc4ccccc45)cc3)c3ccc(-c4ccc5oc6c(-c7ccccc7)cccc6c5c4)cc3)cc2)cc1 ; c1ccc(-c2ccc(N(c3ccc(-c4ccc5c(c4)sc4ccccc45)cc3)c3ccc(-c4ccc5oc6c(-c7ccccc7)cccc6c5c4)cc3)cc2-c2ccccc2)cc1]

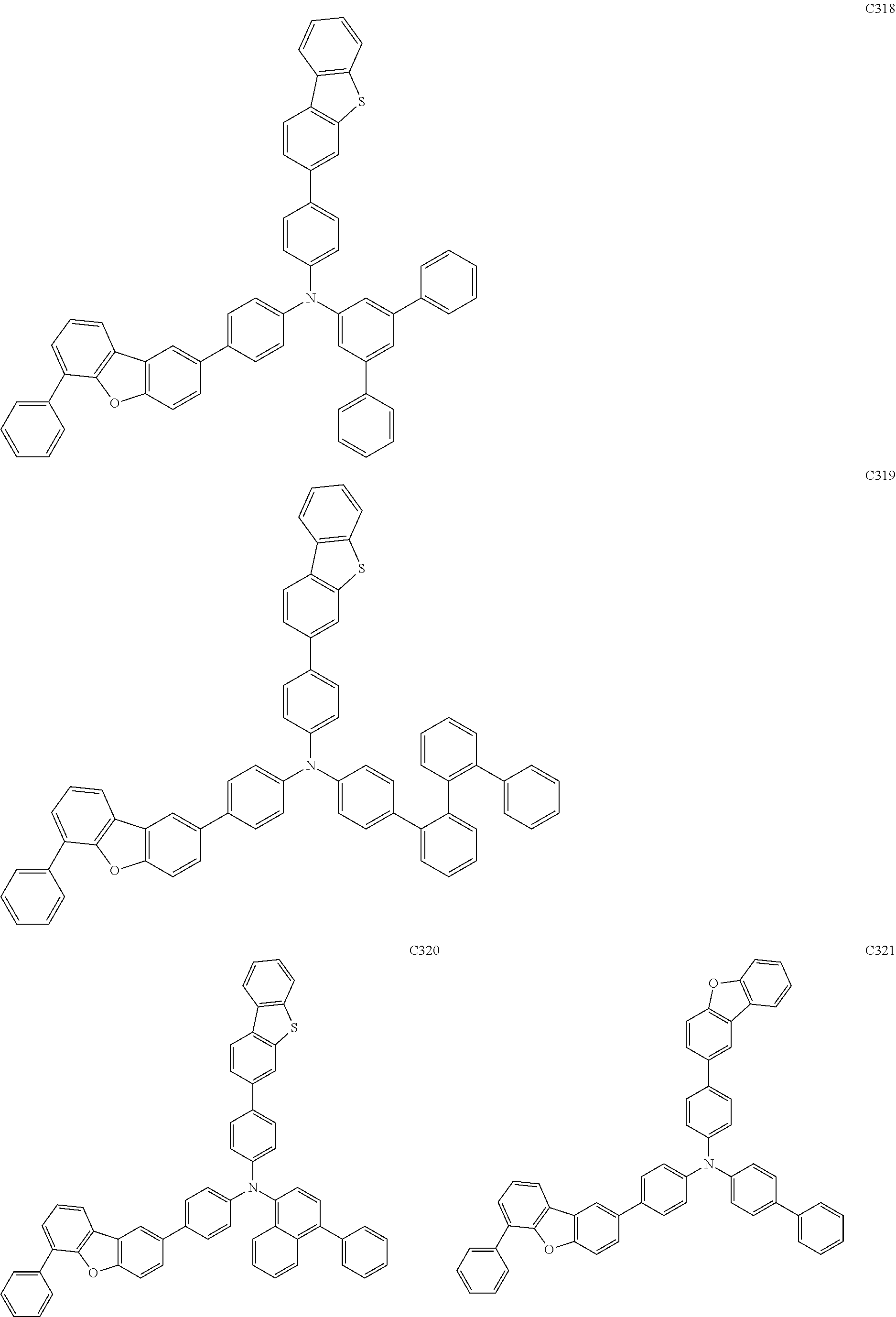

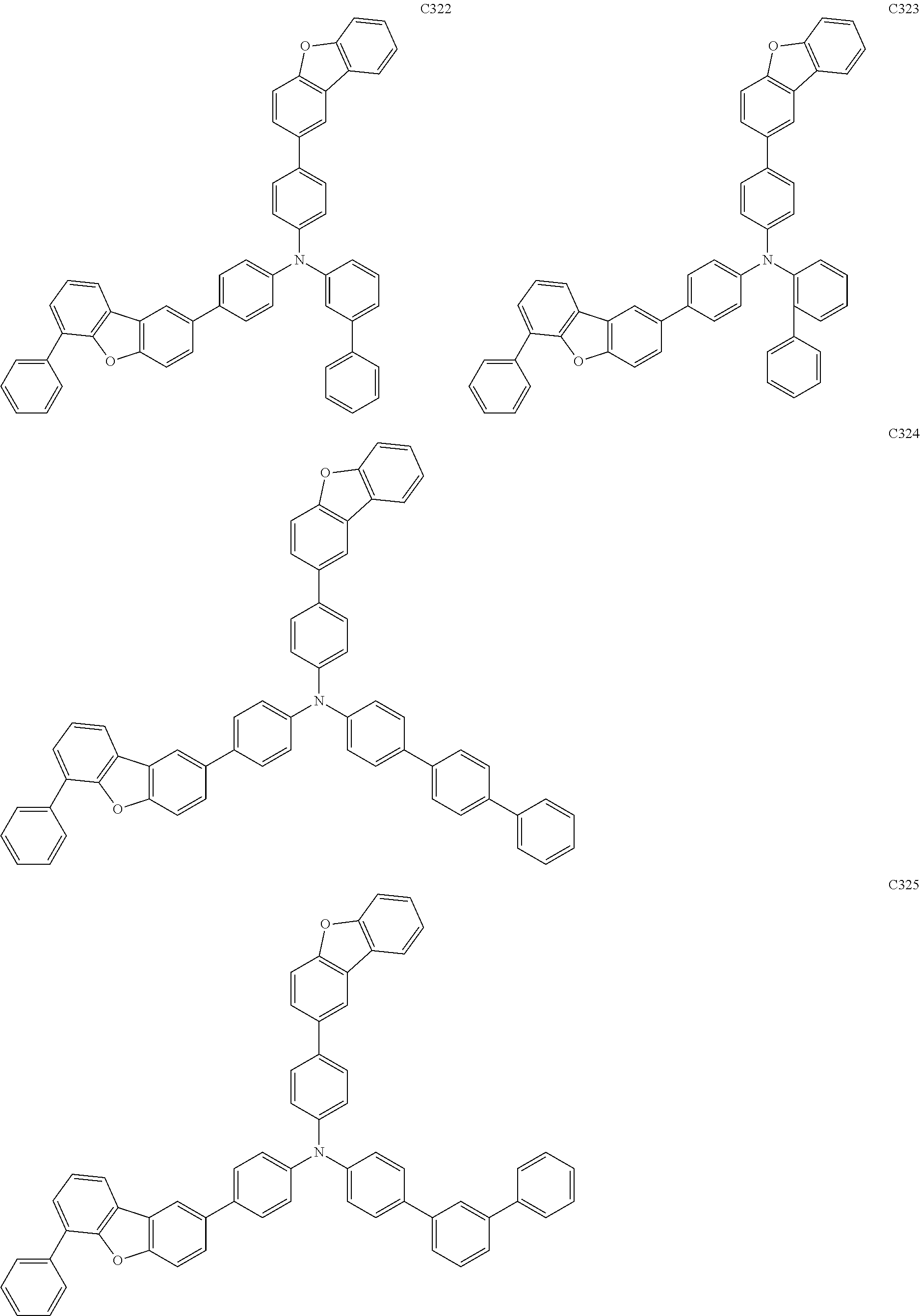

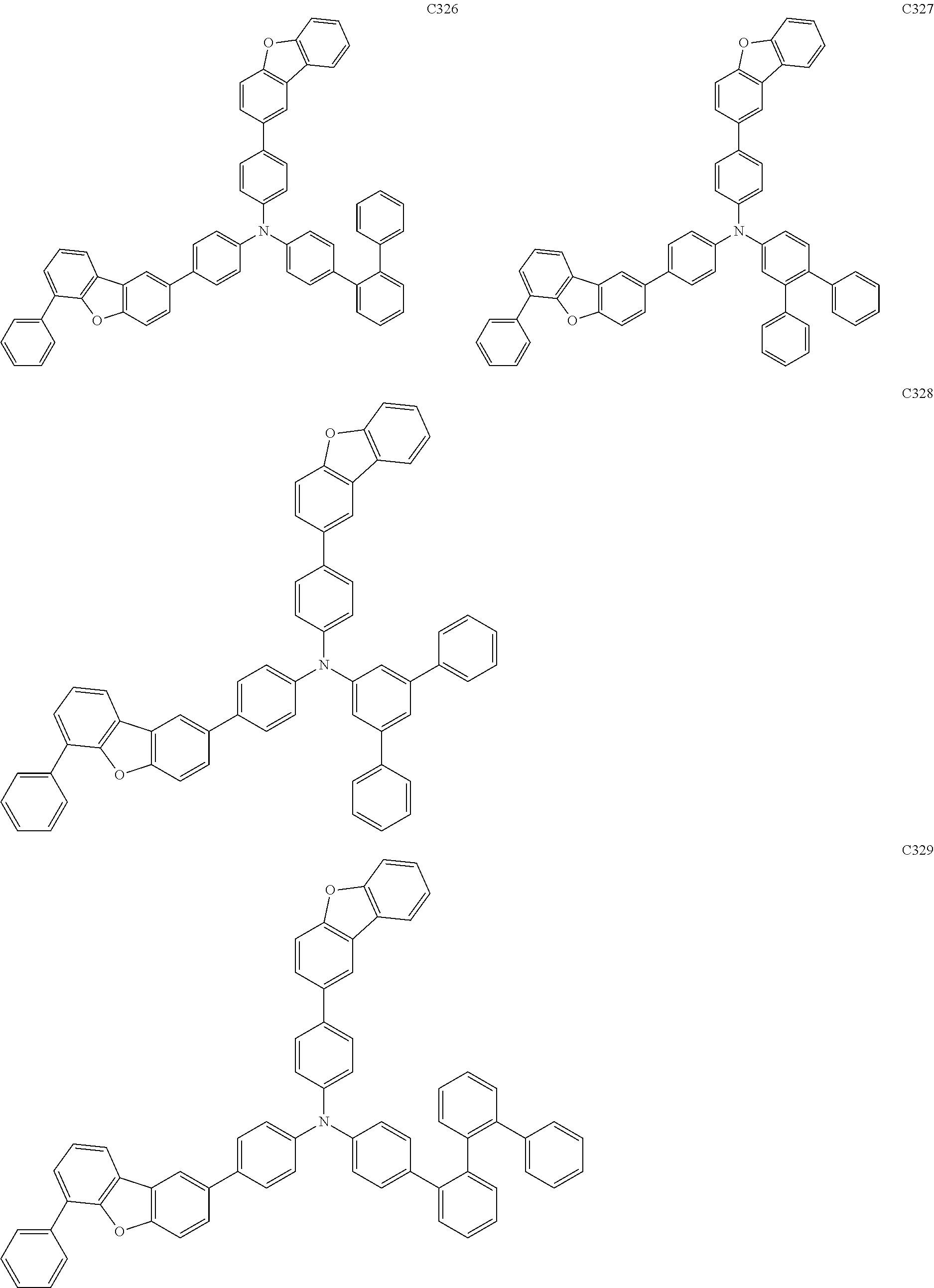

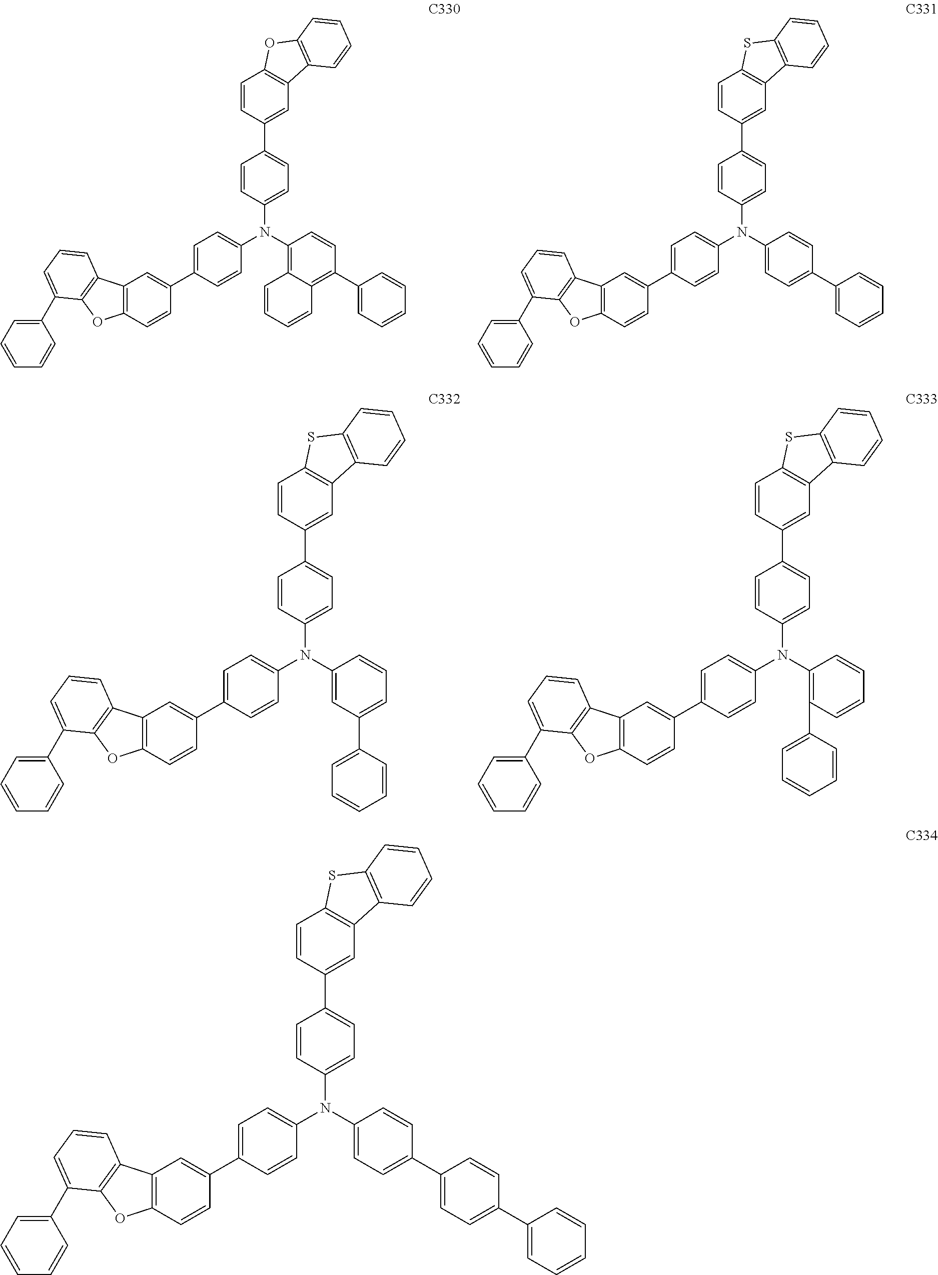

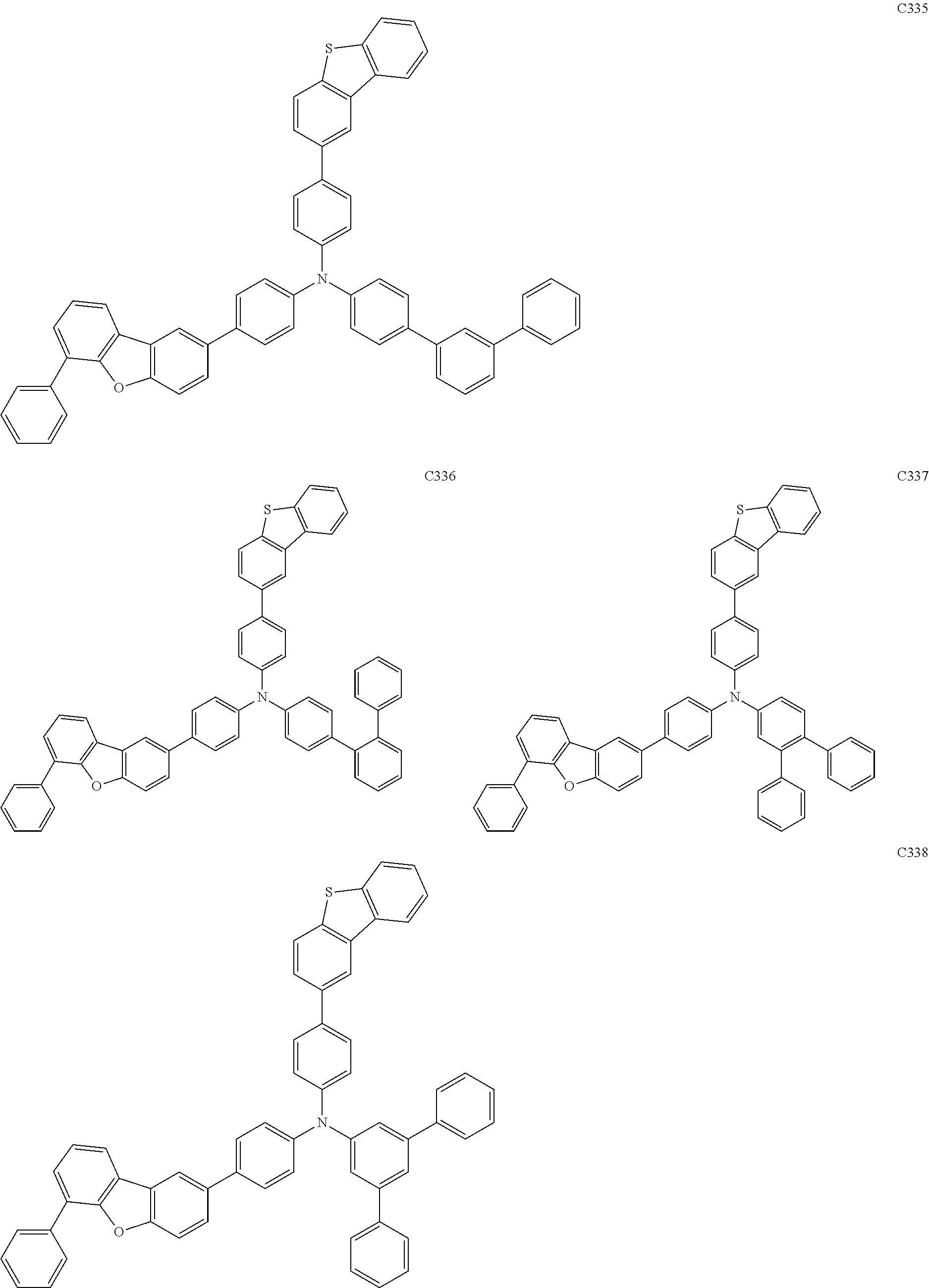 [SMILES: c1ccc(-c2cccc(-c3ccc(N(c4ccc(-c5ccc6oc7c(-c8ccccc8)cccc7c6c5)cc4)c4ccc(-c5ccc6sc7ccccc7c6c5)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4ccc5sc6ccccc6c5c4)cc3)cc2)cc1 ; c1ccc(-c2ccc(N(c3ccc(-c4ccc5oc6c(-c7ccccc7)cccc6c5c4)cc3)c3ccc(-c4ccc5sc6ccccc6c5c4)cc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc(-c4ccc5sc6ccccc6c5c4)cc3)c2)cc1]

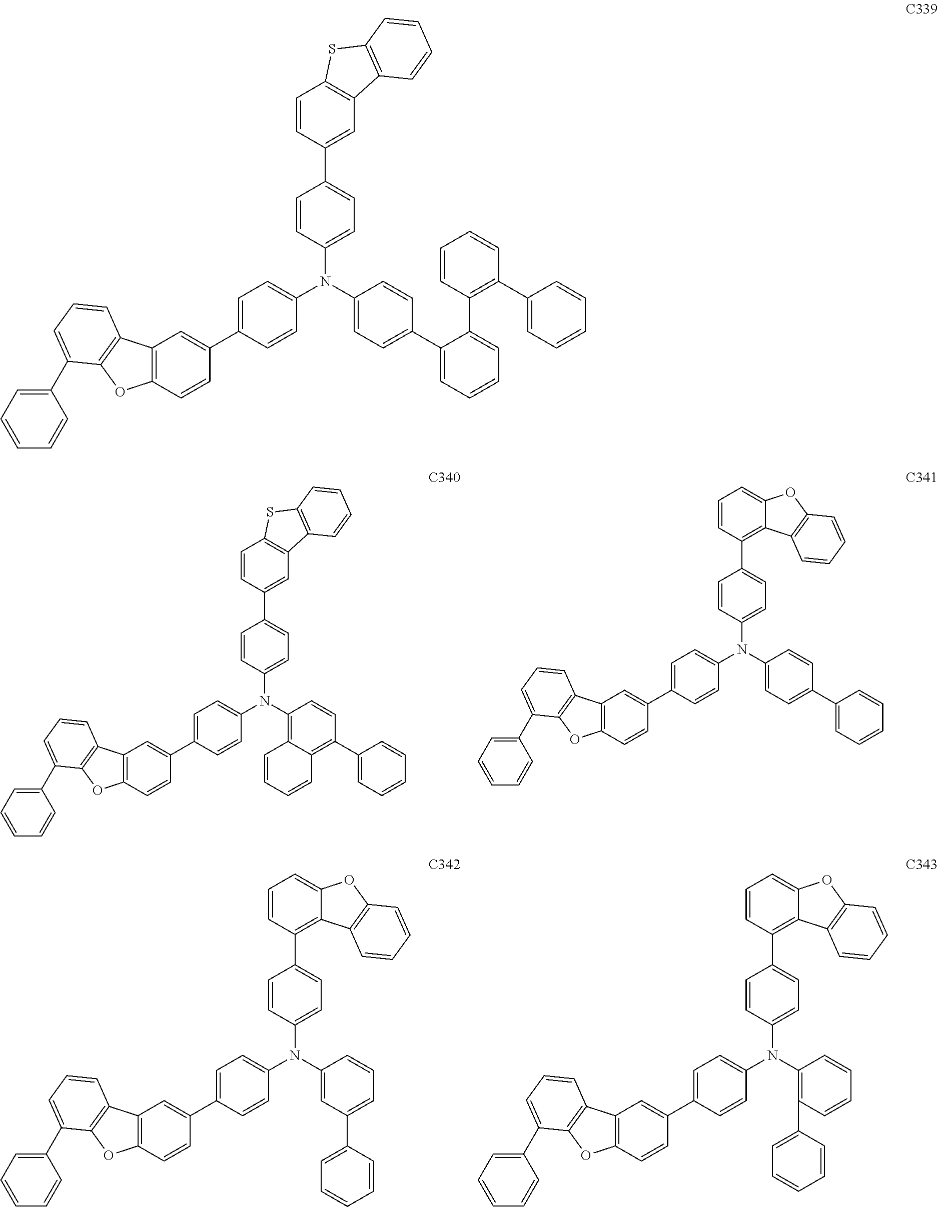

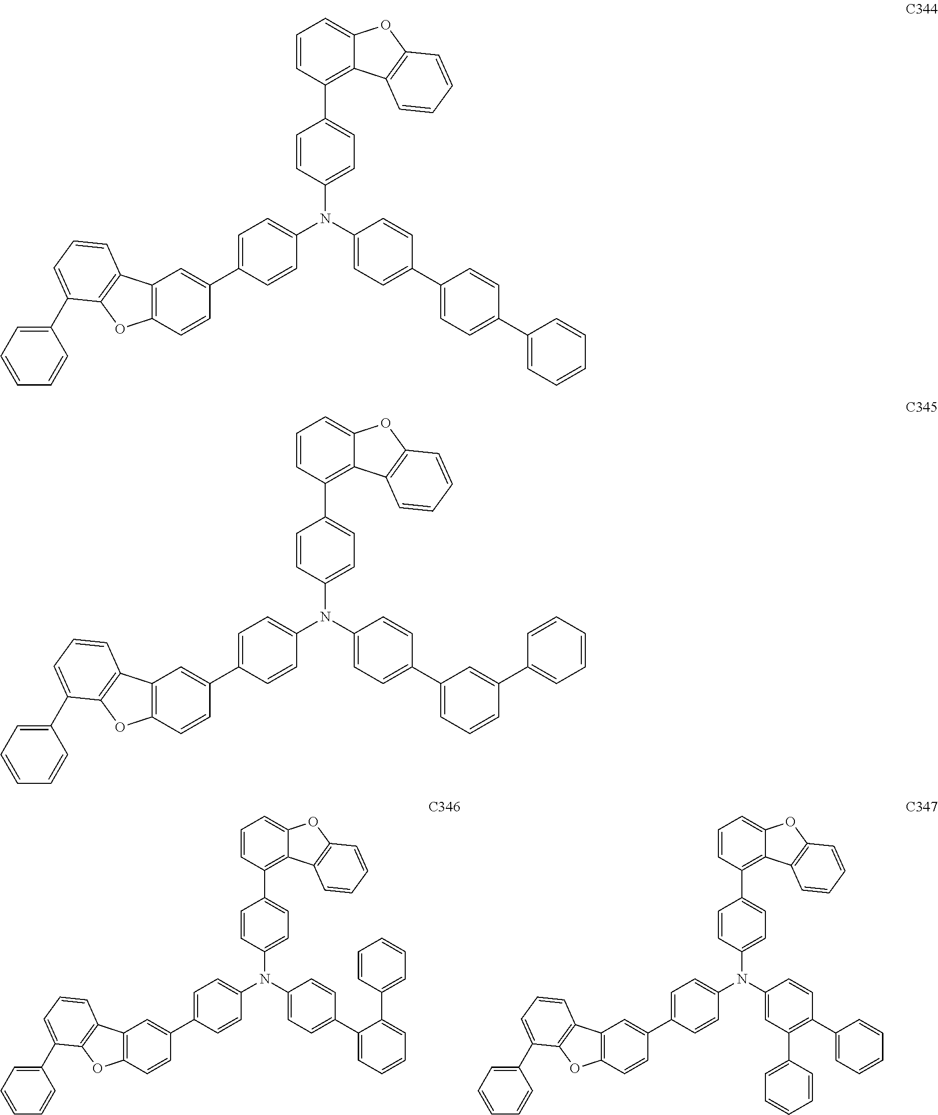 [SMILES: c1ccc(-c2ccc(-c3ccc(N(c4ccc(-c5ccc6oc7c(-c8ccccc8)cccc7c6c5)cc4)c4ccc(-c5cccc6oc7ccccc7c56)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6oc7ccccc7c56)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4cccc5oc6ccccc6c45)cc3)cc2)cc1 ; c1ccc(-c2ccc(N(c3ccc(-c4ccc5oc6c(-c7ccccc7)cccc6c5c4)cc3)c3ccc(-c4cccc5oc6ccccc6c45)cc3)cc2-c2ccccc2)cc1]

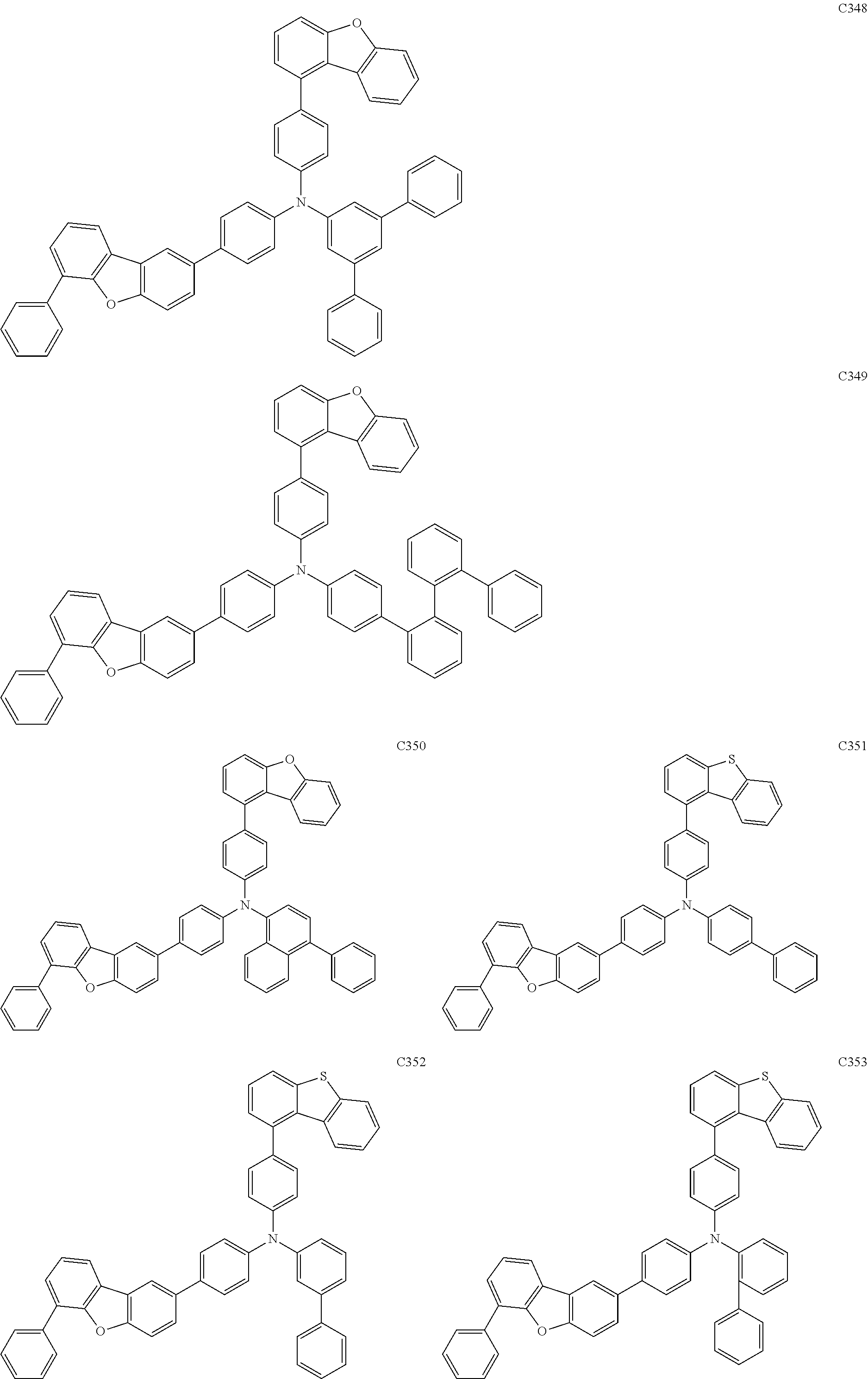

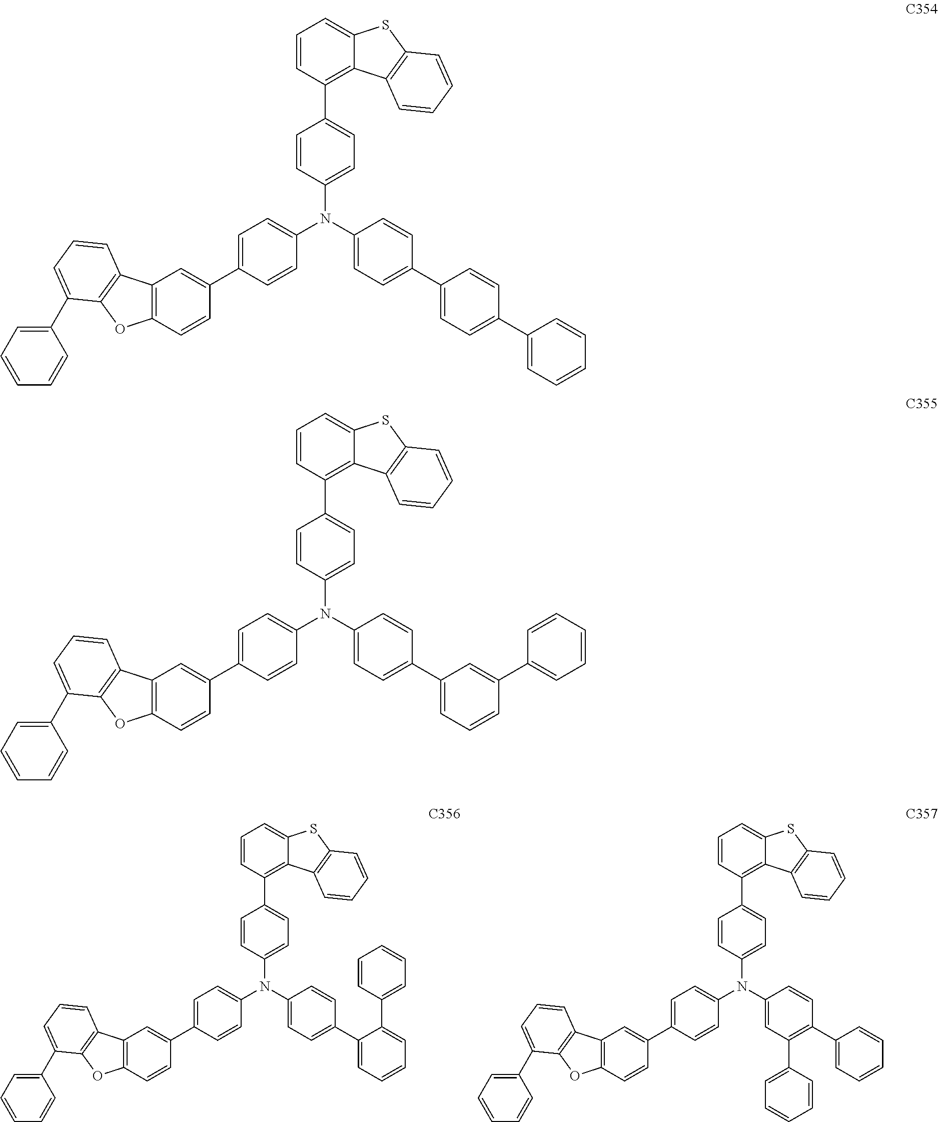 [SMILES: c1ccc(-c2ccc(-c3ccc(N(c4ccc(-c5ccc6oc7c(-c8ccccc8)cccc7c6c5)cc4)c4ccc(-c5cccc6sc7ccccc7c56)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6sc7ccccc7c56)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4cccc5sc6ccccc6c45)cc3)cc2)cc1 ; c1ccc(-c2ccc(N(c3ccc(-c4ccc5oc6c(-c7ccccc7)cccc6c5c4)cc3)c3ccc(-c4cccc5sc6ccccc6c45)cc3)cc2-c2ccccc2)cc1]

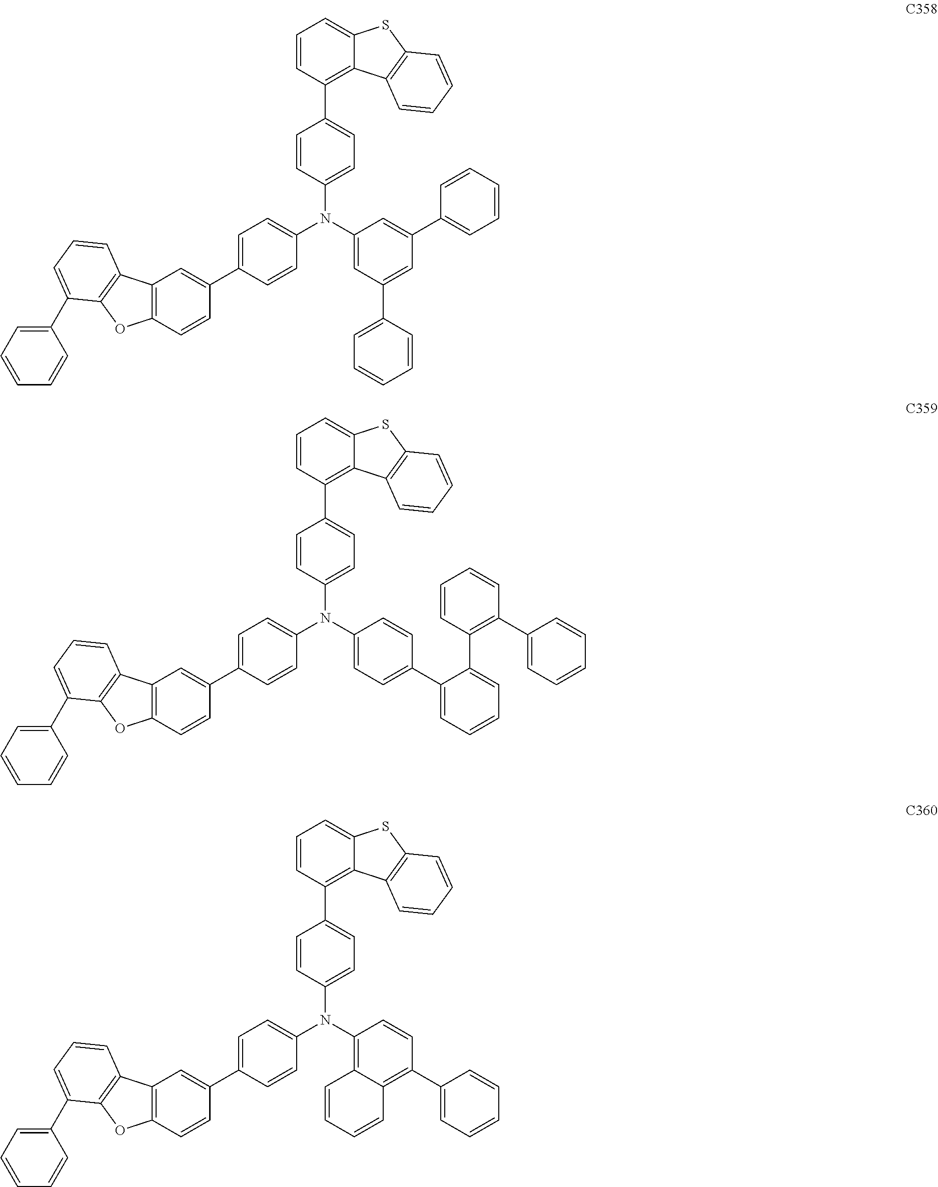

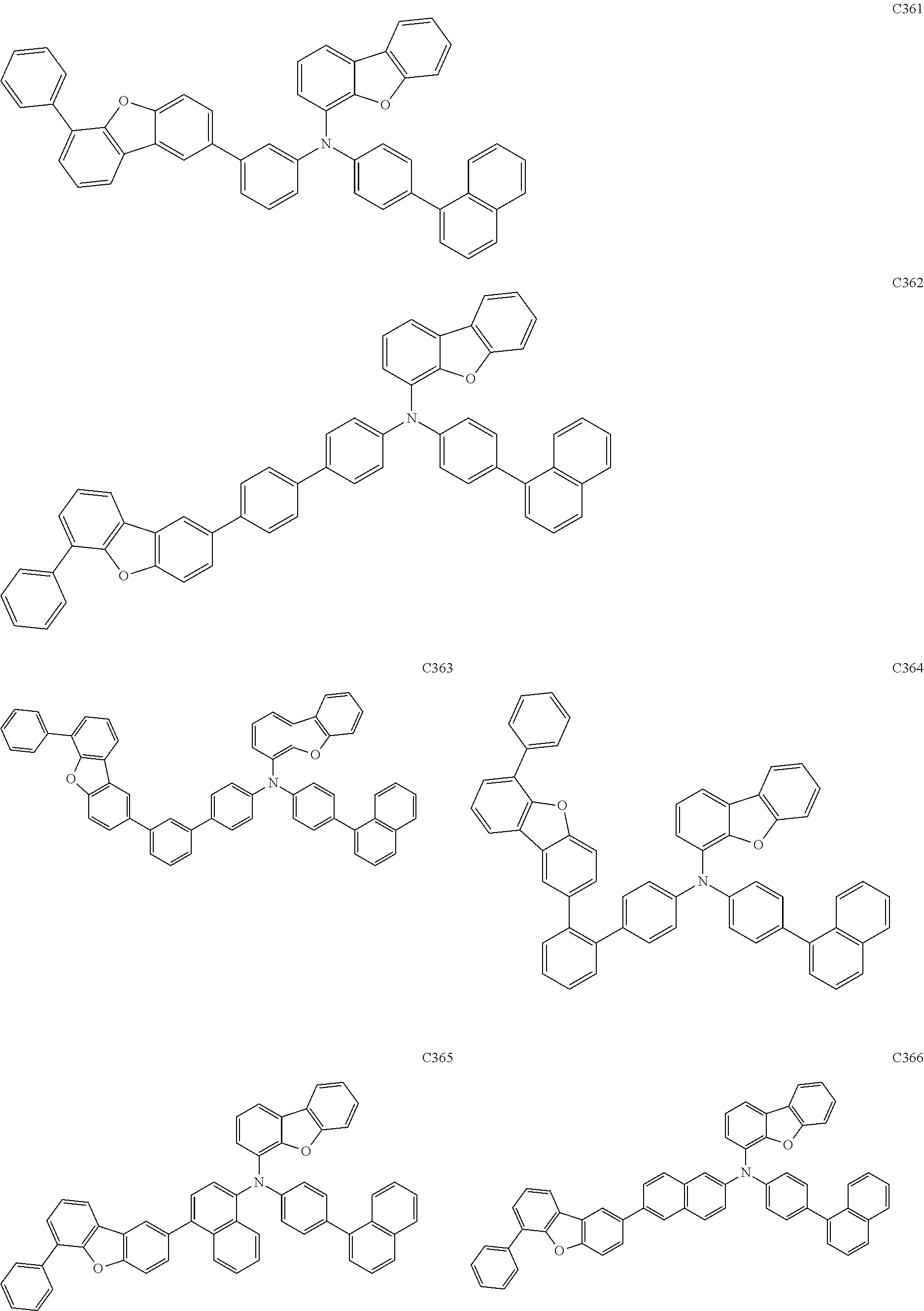

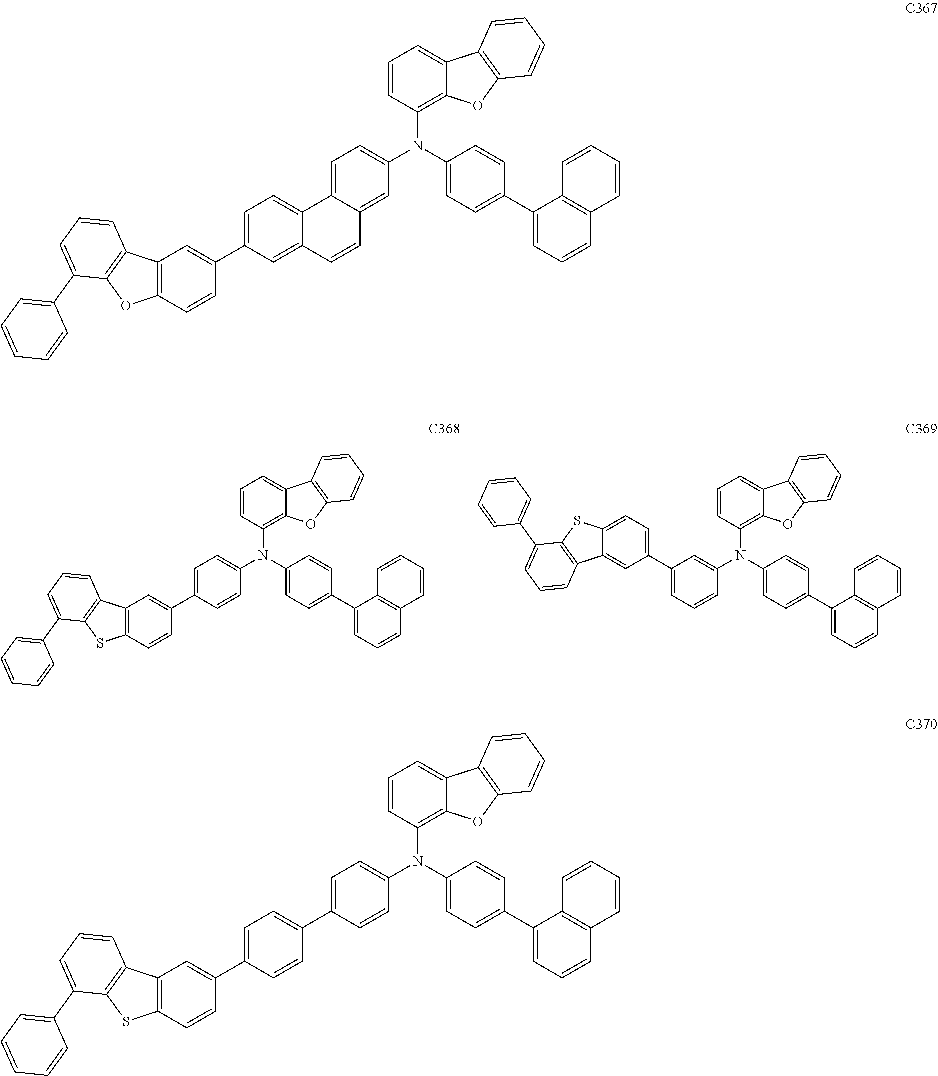 [SMILES: c1ccc(-c2cccc3c2oc2ccc(-c4ccc5c(ccc6cc(N(c7ccc(-c8cccc9ccccc89)cc7)c7cccc8c7oc7ccccc78)ccc65)c4)cc23)cc1 ; c1ccc(-c2cccc3c2sc2ccc(-c4ccc(N(c5ccc(-c6cccc7ccccc67)cc5)c5cccc6c5oc5ccccc56)cc4)cc23)cc1 ; c1ccc(-c2cccc3c2sc2ccc(-c4cccc(N(c5ccc(-c6cccc7ccccc67)cc5)c5cccc6c5oc5ccccc56)c4)cc23)cc1 ; c1ccc(-c2cccc3c2sc2ccc(-c4ccc(-c5ccc(N(c6ccc(-c7cccc8ccccc78)cc6)c6cccc7c6oc6ccccc67)cc5)cc4)cc23)cc1]

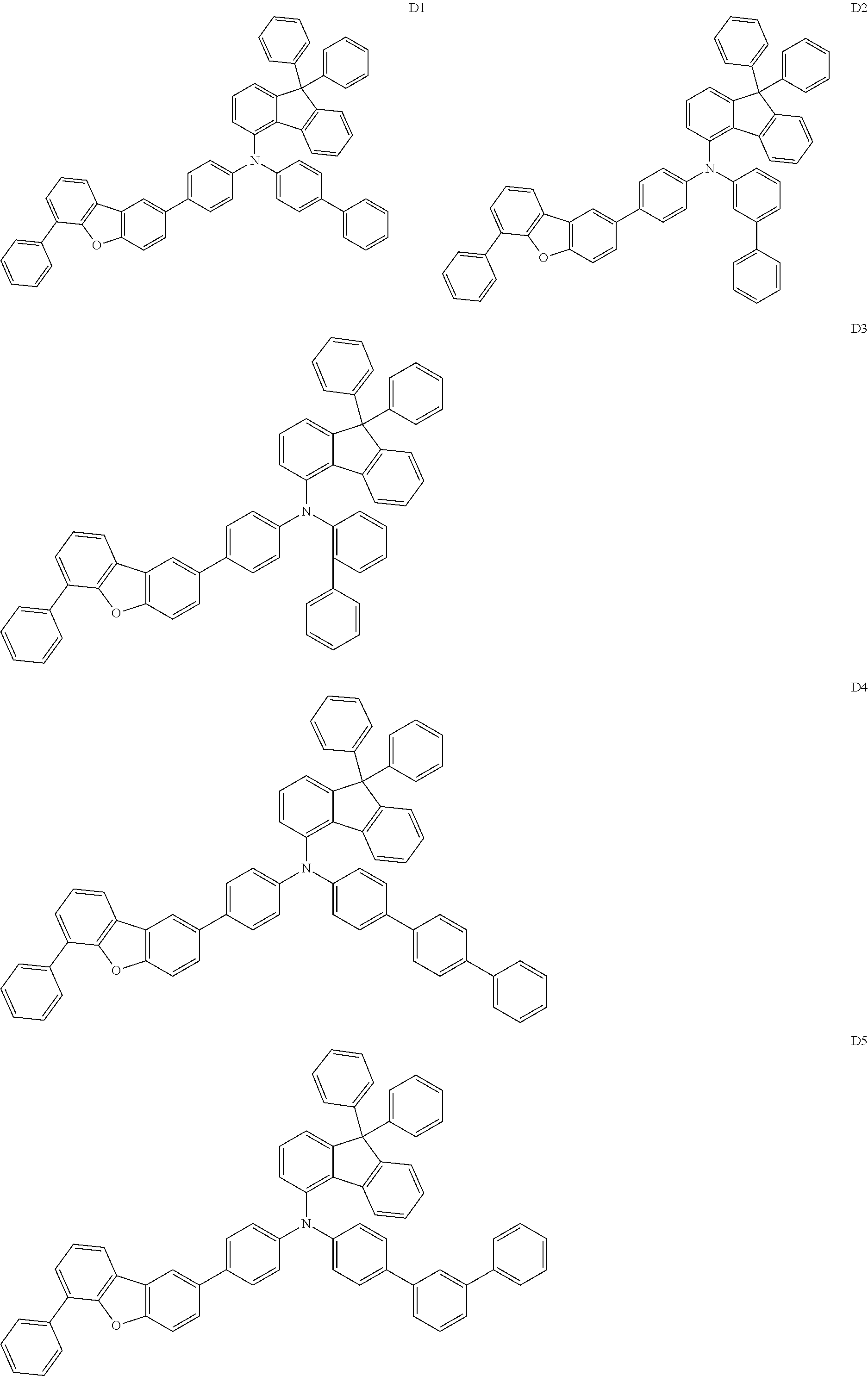

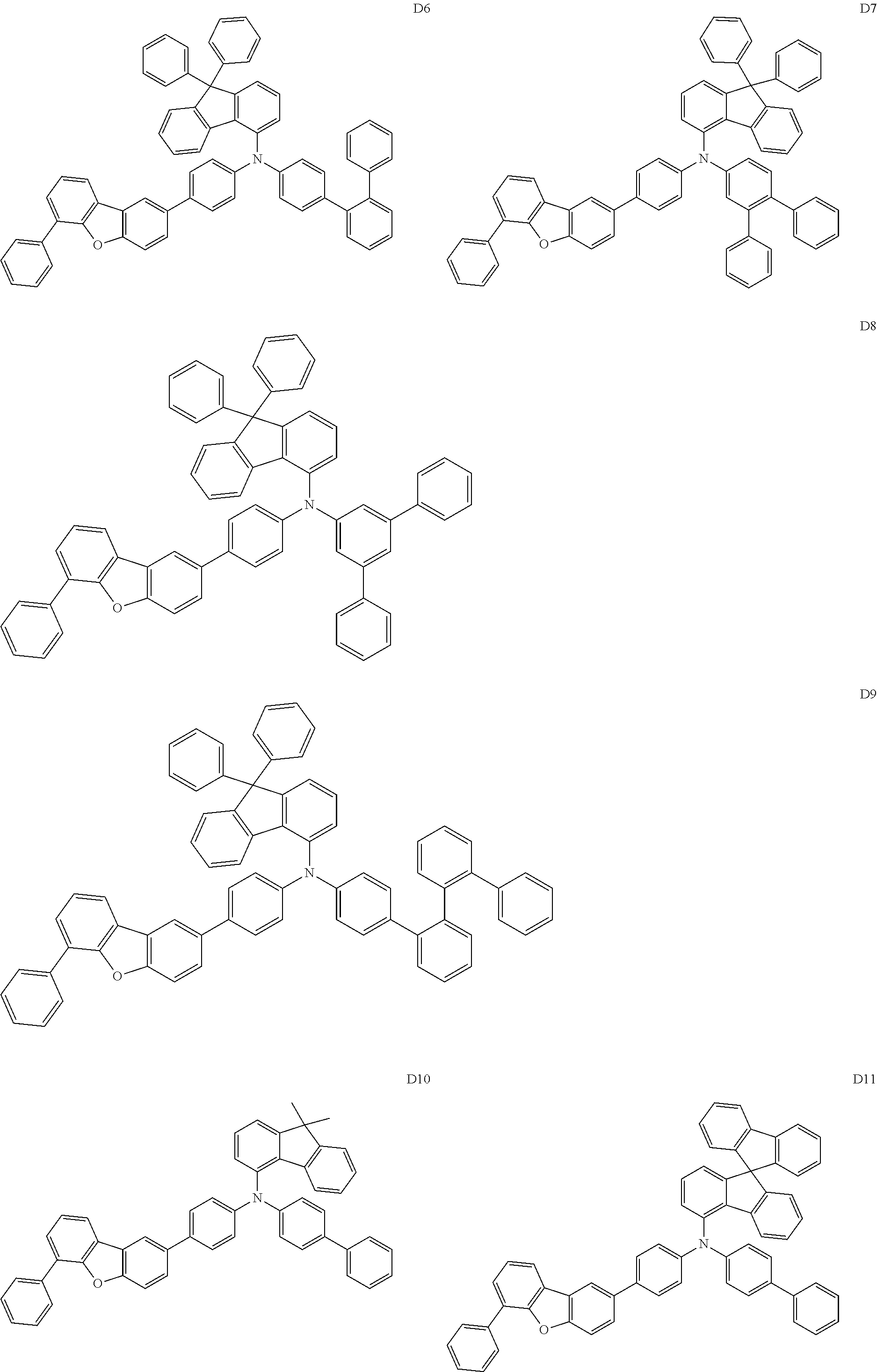

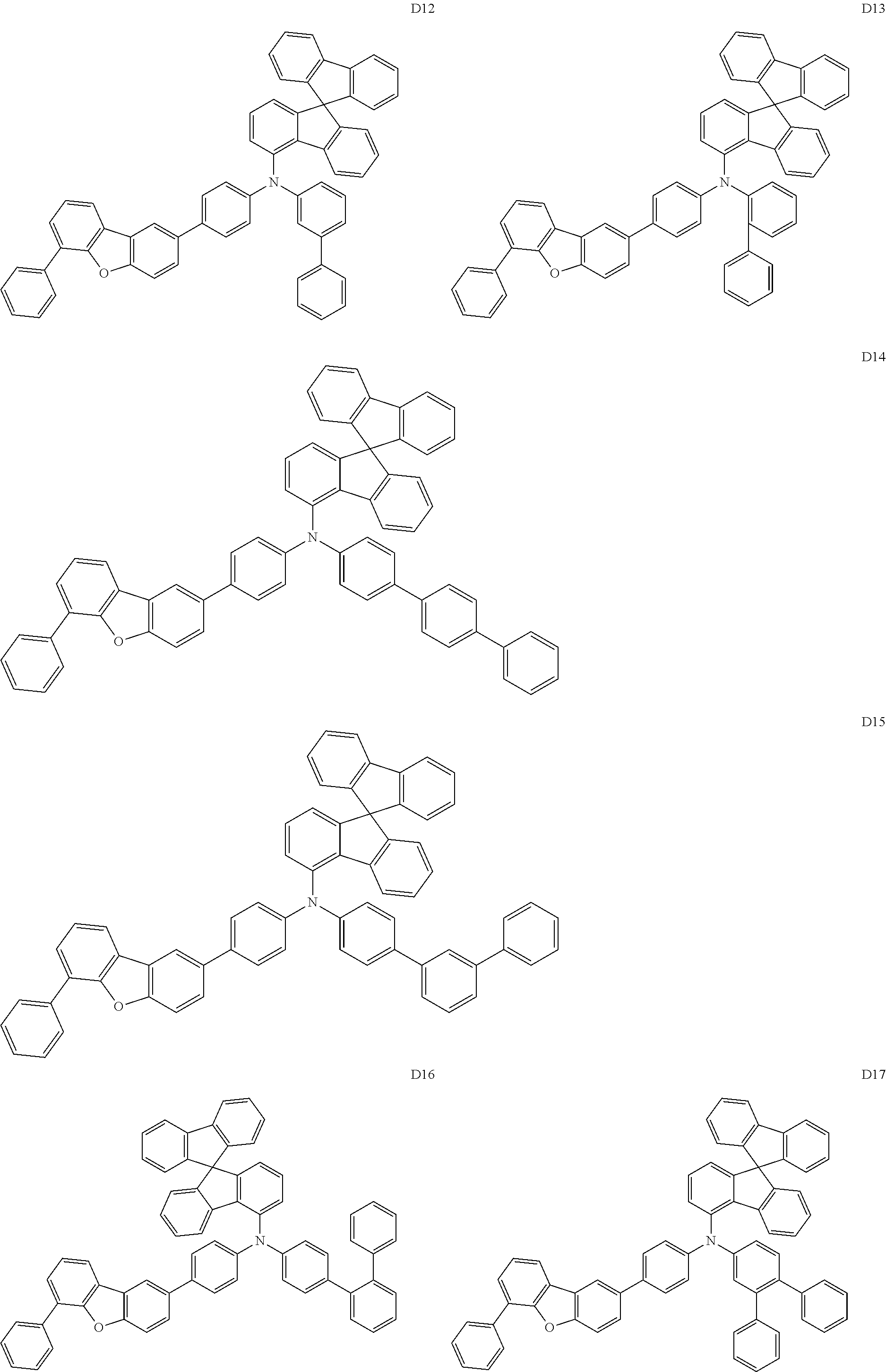

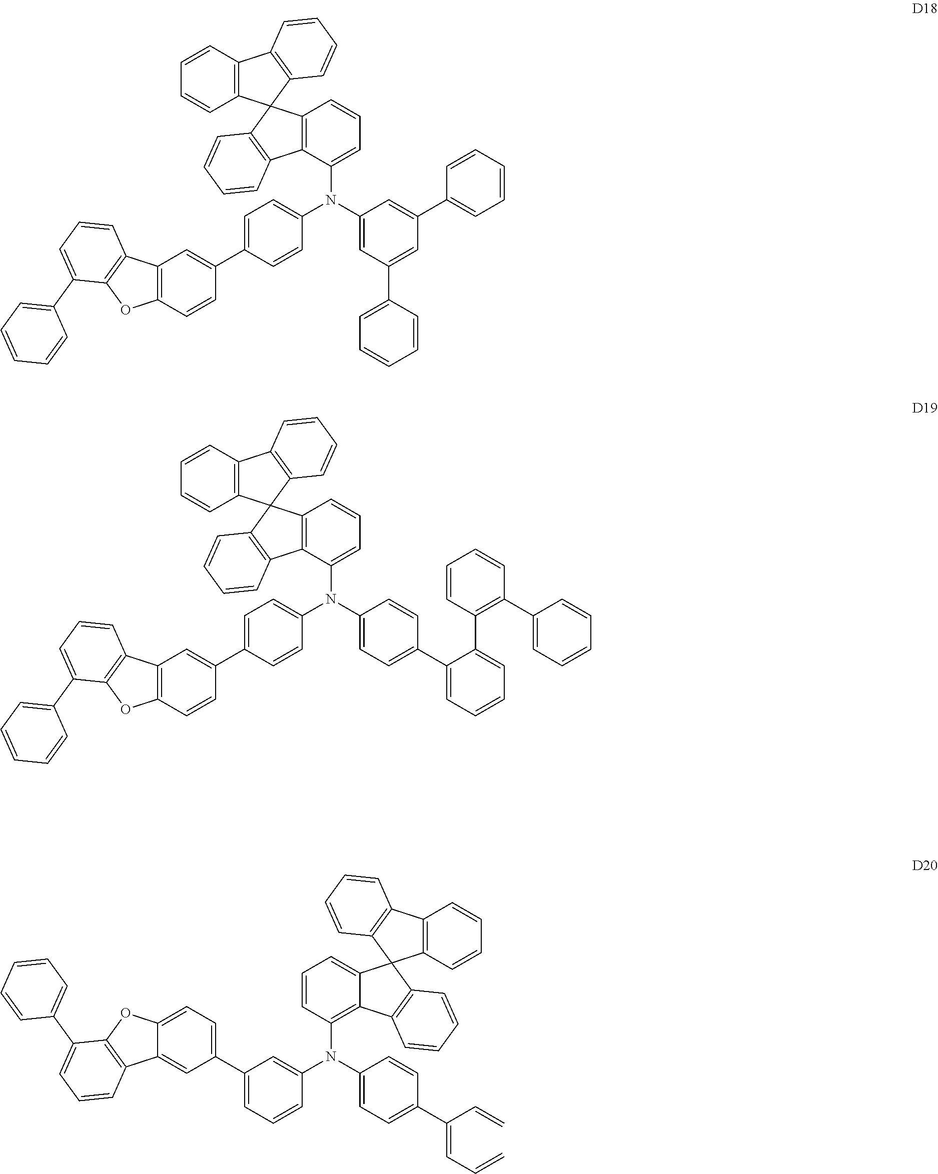 [SMILES: c1ccc(-c2cc(-c3ccccc3)cc(N(c3ccc(-c4ccc5oc6c(-c7ccccc7)cccc6c5c4)cc3)c3cccc4c3-c3ccccc3C43c4ccccc4-c4ccccc43)c2)cc1 ; c1ccc(-c2ccccc2-c2ccccc2-c2ccc(N(c3ccc(-c4ccc5oc6c(-c7ccccc7)cccc6c5c4)cc3)c3cccc4c3-c3ccccc3C43c4ccccc4-c4ccccc43)cc2)cc1 ; C=C/C=C(\C=C)c1ccc(N(c2cccc(-c3ccc4oc5c(-c6ccccc6)cccc5c4c3)c2)c2cccc3c2-c2ccccc2C32c3ccccc3-c3ccccc32)cc1]

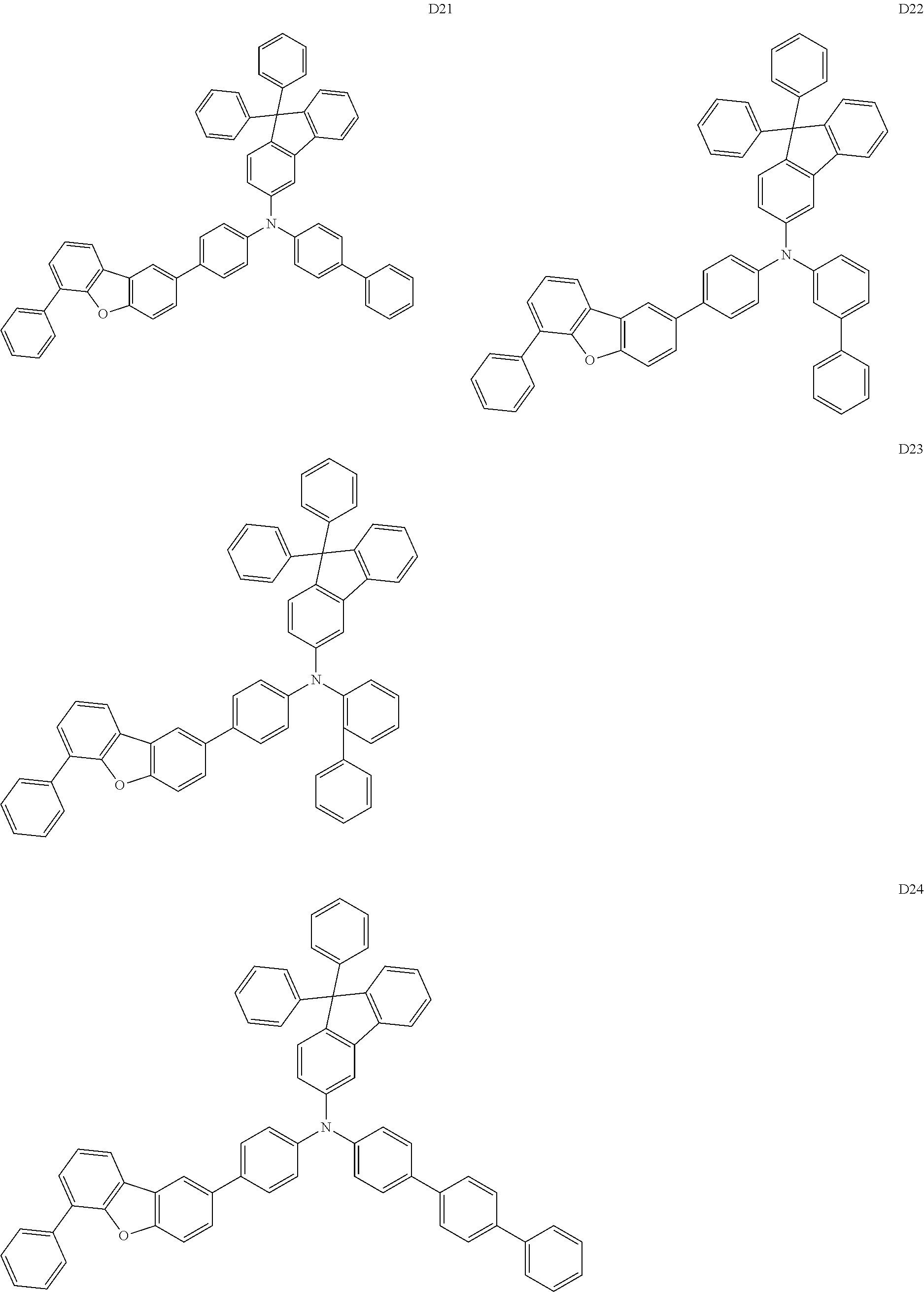 [SMILES: c1ccc(-c2ccc(N(c3ccc(-c4ccc5oc6c(-c7ccccc7)cccc6c5c4)cc3)c3ccc4c(c3)-c3ccccc3C4(c3ccccc3)c3ccccc3)cc2)cc1 ; c1ccc(-c2cccc(N(c3ccc(-c4ccc5oc6c(-c7ccccc7)cccc6c5c4)cc3)c3ccc4c(c3)-c3ccccc3C4(c3ccccc3)c3ccccc3)c2)cc1 ; c1ccc(-c2ccccc2N(c2ccc(-c3ccc4oc5c(-c6ccccc6)cccc5c4c3)cc2)c2ccc3c(c2)-c2ccccc2C3(c2ccccc2)c2ccccc2)cc1 ; c1ccc(-c2ccc(-c3ccc(N(c4ccc(-c5ccc6oc7c(-c8ccccc8)cccc7c6c5)cc4)c4ccc5c(c4)-c4ccccc4C5(c4ccccc4)c4ccccc4)cc3)cc2)cc1]

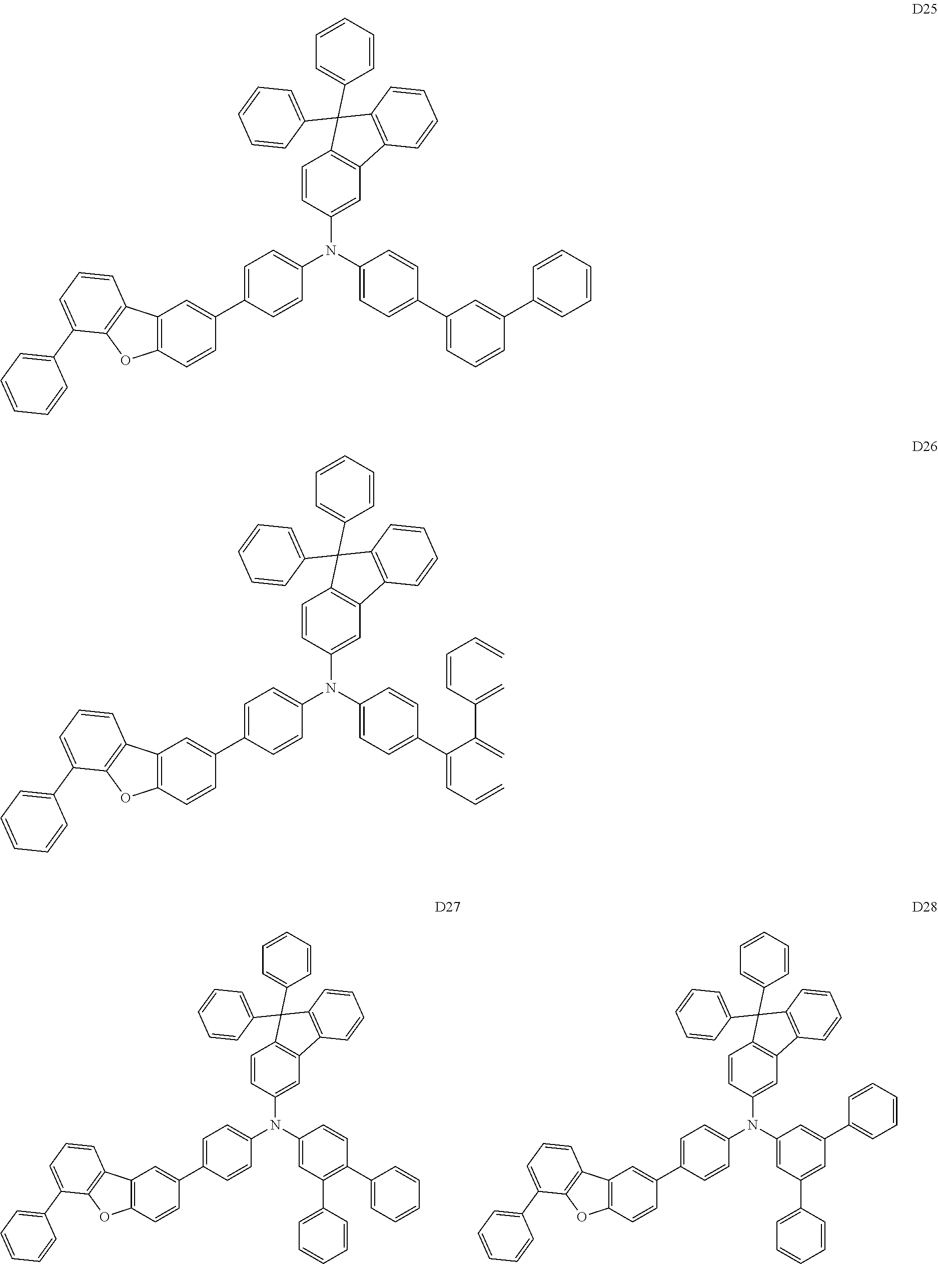 [SMILES: c1ccc(-c2cccc(-c3ccc(N(c4ccc(-c5ccc6oc7c(-c8ccccc8)cccc7c6c5)cc4)c4ccc5c(c4)-c4ccccc4C5(c4ccccc4)c4ccccc4)cc3)c2)cc1 ; C=C/C=C\C(=C)C(=C)/C(=C\C=C)c1ccc(N(c2ccc(-c3ccc4oc5c(-c6ccccc6)cccc5c4c3)cc2)c2ccc3c(c2)-c2ccccc2C3(c2ccccc2)c2ccccc2)cc1 ; c1ccc(-c2ccc(N(c3ccc(-c4ccc5oc6c(-c7ccccc7)cccc6c5c4)cc3)c3ccc4c(c3)-c3ccccc3C4(c3ccccc3)c3ccccc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc4c(c3)-c3ccccc3C4(c3ccccc3)c3ccccc3)c2)cc1]

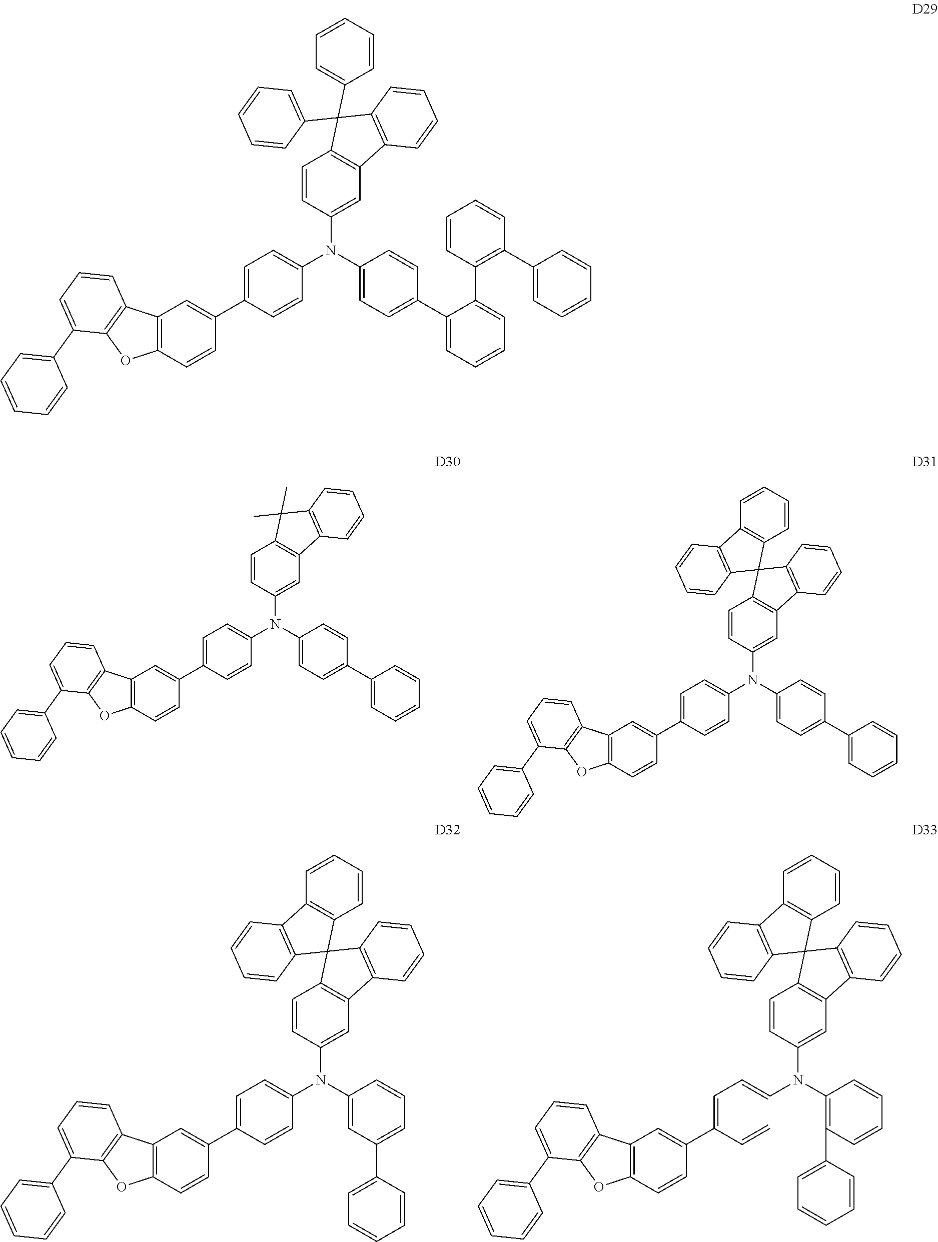

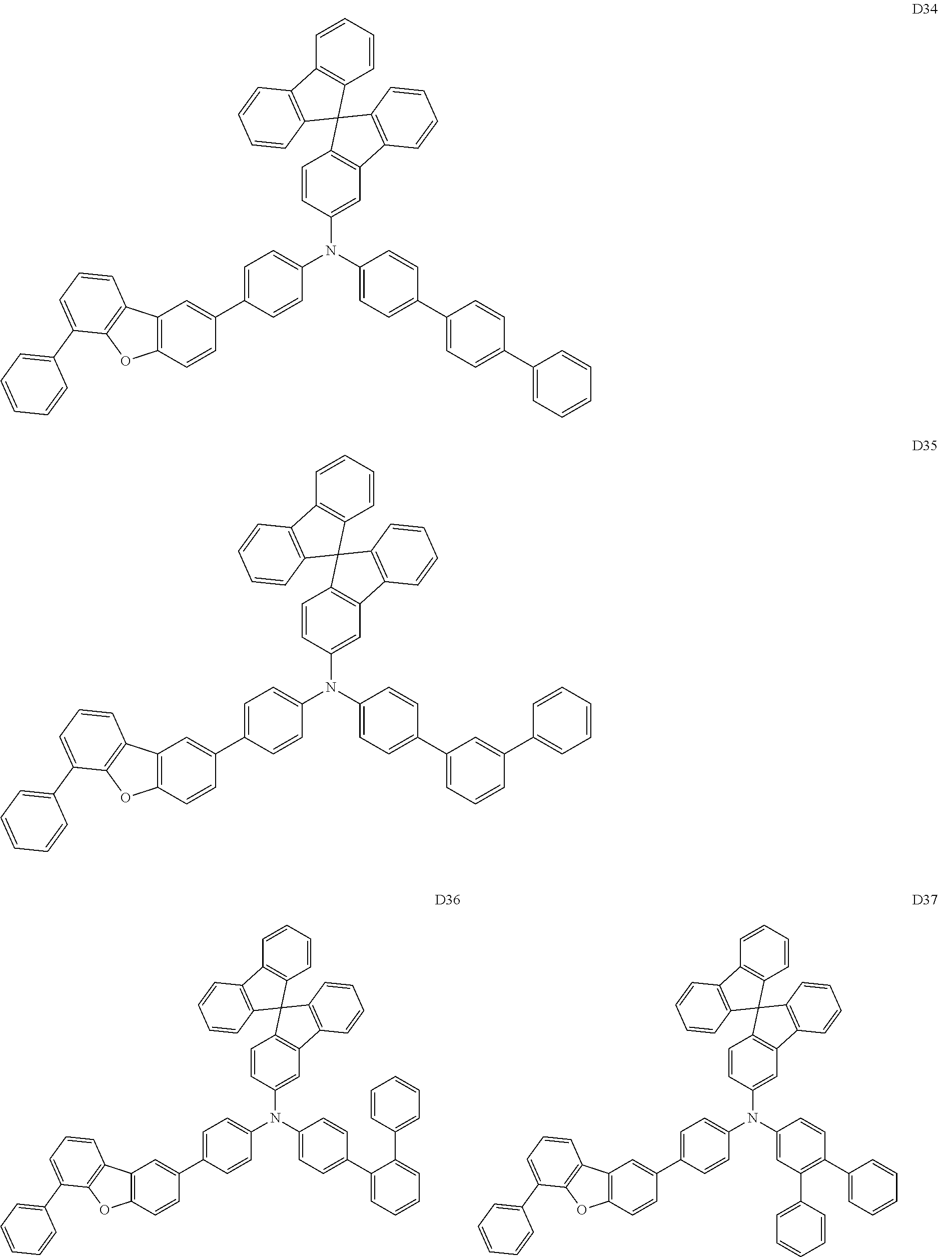 [SMILES: c1ccc(-c2ccc(-c3ccc(N(c4ccc(-c5ccc6oc7c(-c8ccccc8)cccc7c6c5)cc4)c4ccc5c(c4)-c4ccccc4C54c5ccccc5-c5ccccc54)cc3)cc2)cc1 ; c1ccc(-c2cccc(-c3ccc(N(c4ccc(-c5ccc6oc7c(-c8ccccc8)cccc7c6c5)cc4)c4ccc5c(c4)-c4ccccc4C54c5ccccc5-c5ccccc54)cc3)c2)cc1 ; c1ccc(-c2ccccc2-c2ccc(N(c3ccc(-c4ccc5oc6c(-c7ccccc7)cccc6c5c4)cc3)c3ccc4c(c3)-c3ccccc3C43c4ccccc4-c4ccccc43)cc2)cc1 ; c1ccc(-c2ccc(N(c3ccc(-c4ccc5oc6c(-c7ccccc7)cccc6c5c4)cc3)c3ccc4c(c3)-c3ccccc3C43c4ccccc4-c4ccccc43)cc2-c2ccccc2)cc1]

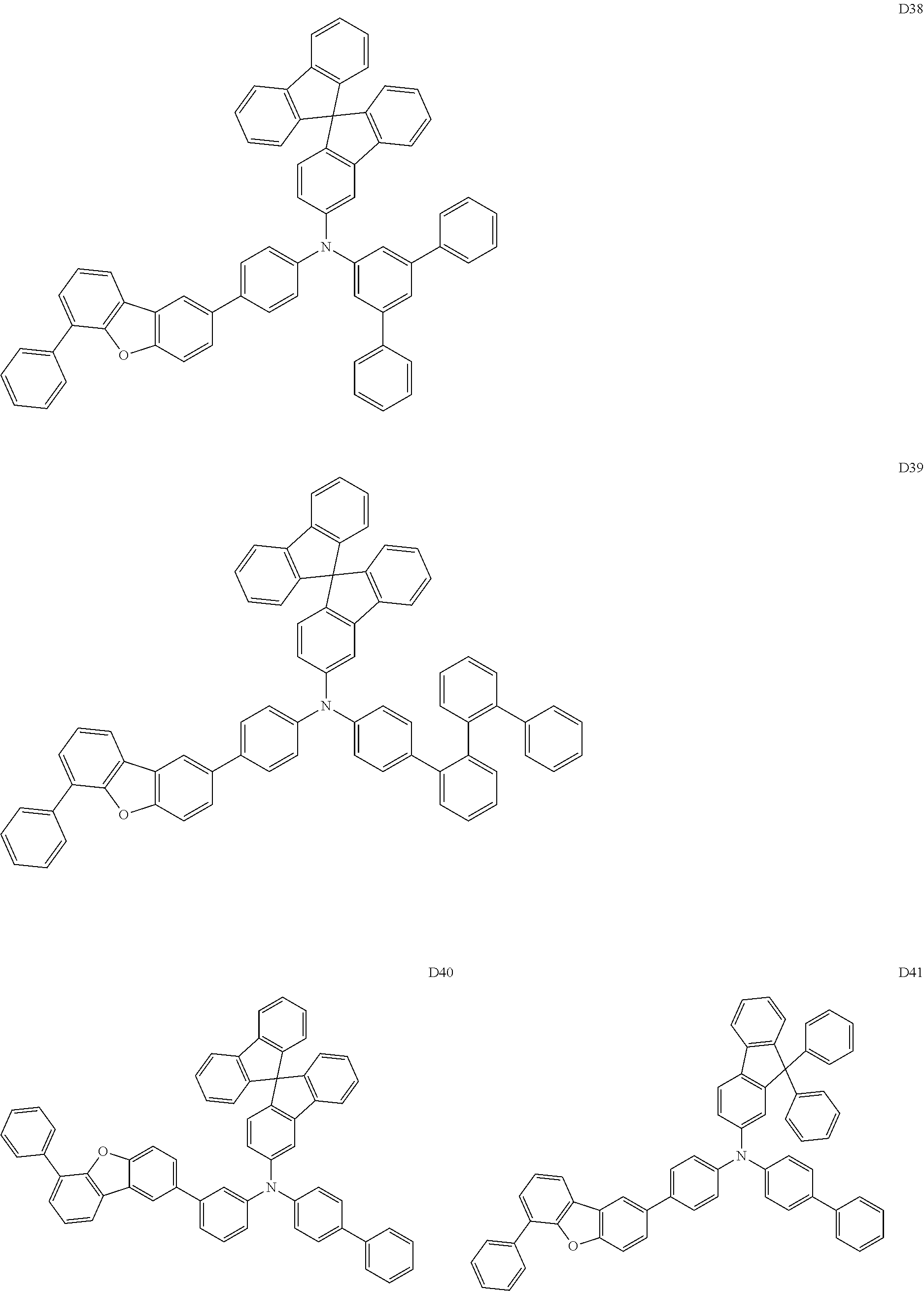 [SMILES: c1ccc(-c2cc(-c3ccccc3)cc(N(c3ccc(-c4ccc5oc6c(-c7ccccc7)cccc6c5c4)cc3)c3ccc4c(c3)-c3ccccc3C43c4ccccc4-c4ccccc43)c2)cc1 ; c1ccc(-c2ccccc2-c2ccccc2-c2ccc(N(c3ccc(-c4ccc5oc6c(-c7ccccc7)cccc6c5c4)cc3)c3ccc4c(c3)-c3ccccc3C43c4ccccc4-c4ccccc43)cc2)cc1 ; c1ccc(-c2ccc(N(c3cccc(-c4ccc5oc6c(-c7ccccc7)cccc6c5c4)c3)c3ccc4c(c3)-c3ccccc3C43c4ccccc4-c4ccccc43)cc2)cc1 ; c1ccc(-c2ccc(N(c3ccc(-c4ccc5oc6c(-c7ccccc7)cccc6c5c4)cc3)c3ccc4c(c3)C(c3ccccc3)(c3ccccc3)c3ccccc3-4)cc2)cc1]

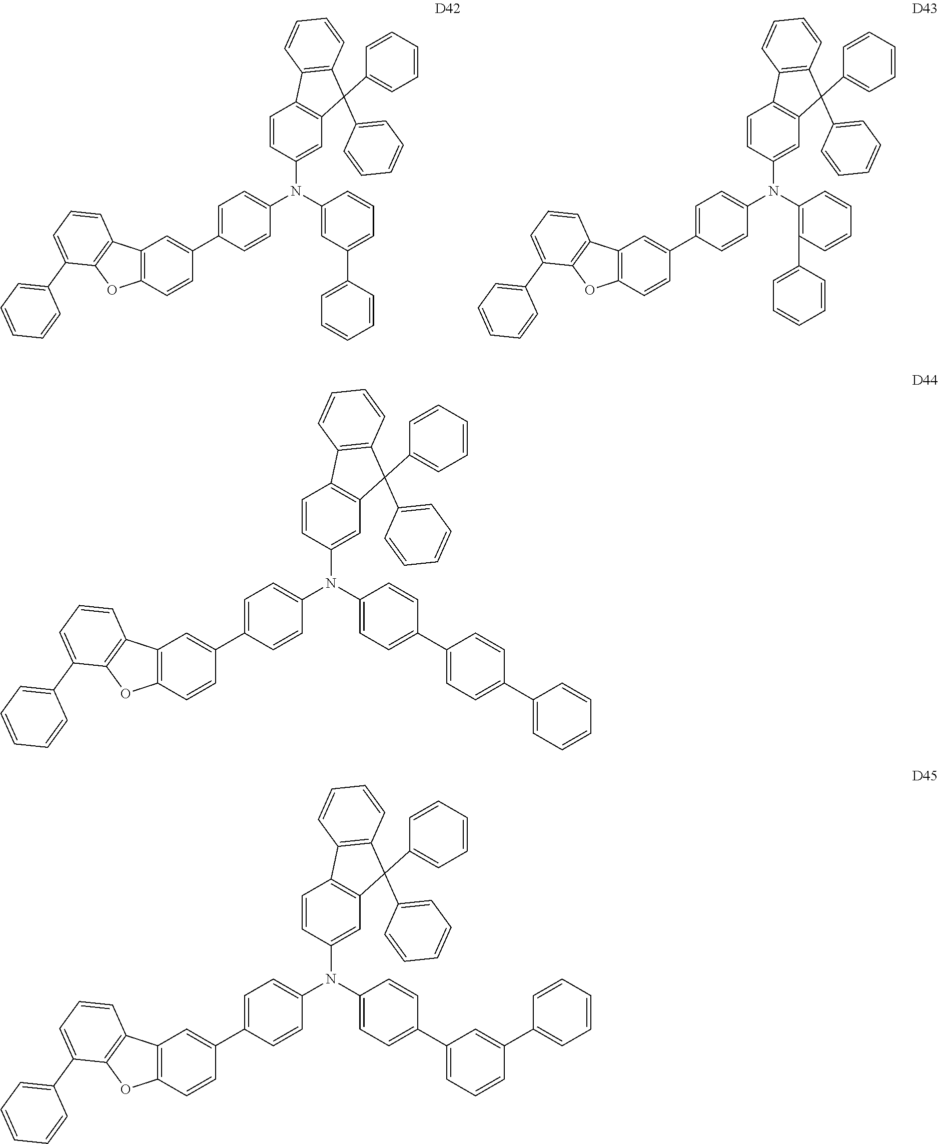 [SMILES: c1ccc(-c2cccc(N(c3ccc(-c4ccc5oc6c(-c7ccccc7)cccc6c5c4)cc3)c3ccc4c(c3)C(c3ccccc3)(c3ccccc3)c3ccccc3-4)c2)cc1 ; c1ccc(-c2ccccc2N(c2ccc(-c3ccc4oc5c(-c6ccccc6)cccc5c4c3)cc2)c2ccc3c(c2)C(c2ccccc2)(c2ccccc2)c2ccccc2-3)cc1 ; c1ccc(-c2ccc(-c3ccc(N(c4ccc(-c5ccc6oc7c(-c8ccccc8)cccc7c6c5)cc4)c4ccc5c(c4)C(c4ccccc4)(c4ccccc4)c4ccccc4-5)cc3)cc2)cc1 ; c1ccc(-c2cccc(-c3ccc(N(c4ccc(-c5ccc6oc7c(-c8ccccc8)cccc7c6c5)cc4)c4ccc5c(c4)C(c4ccccc4)(c4ccccc4)c4ccccc4-5)cc3)c2)cc1]

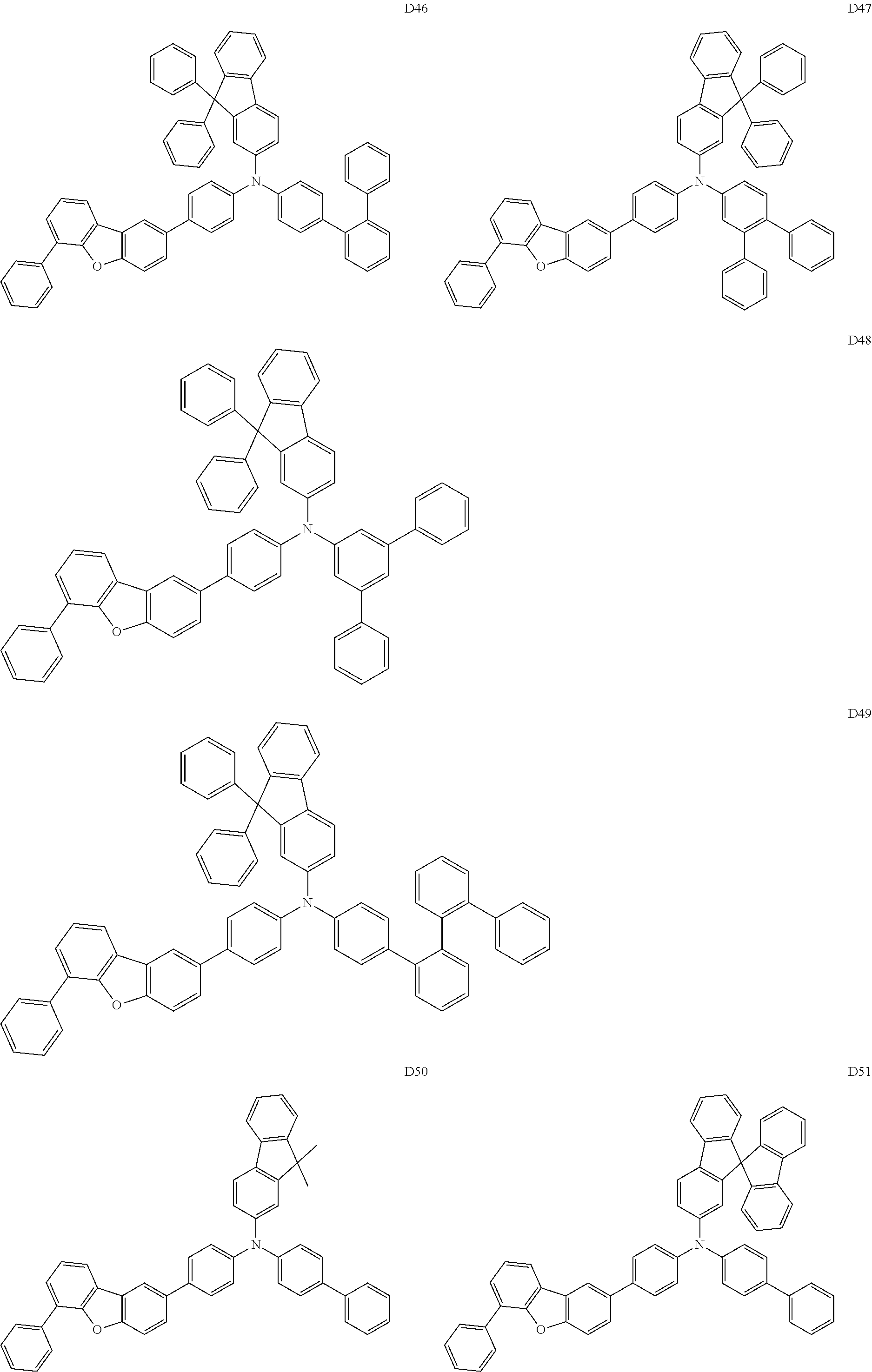

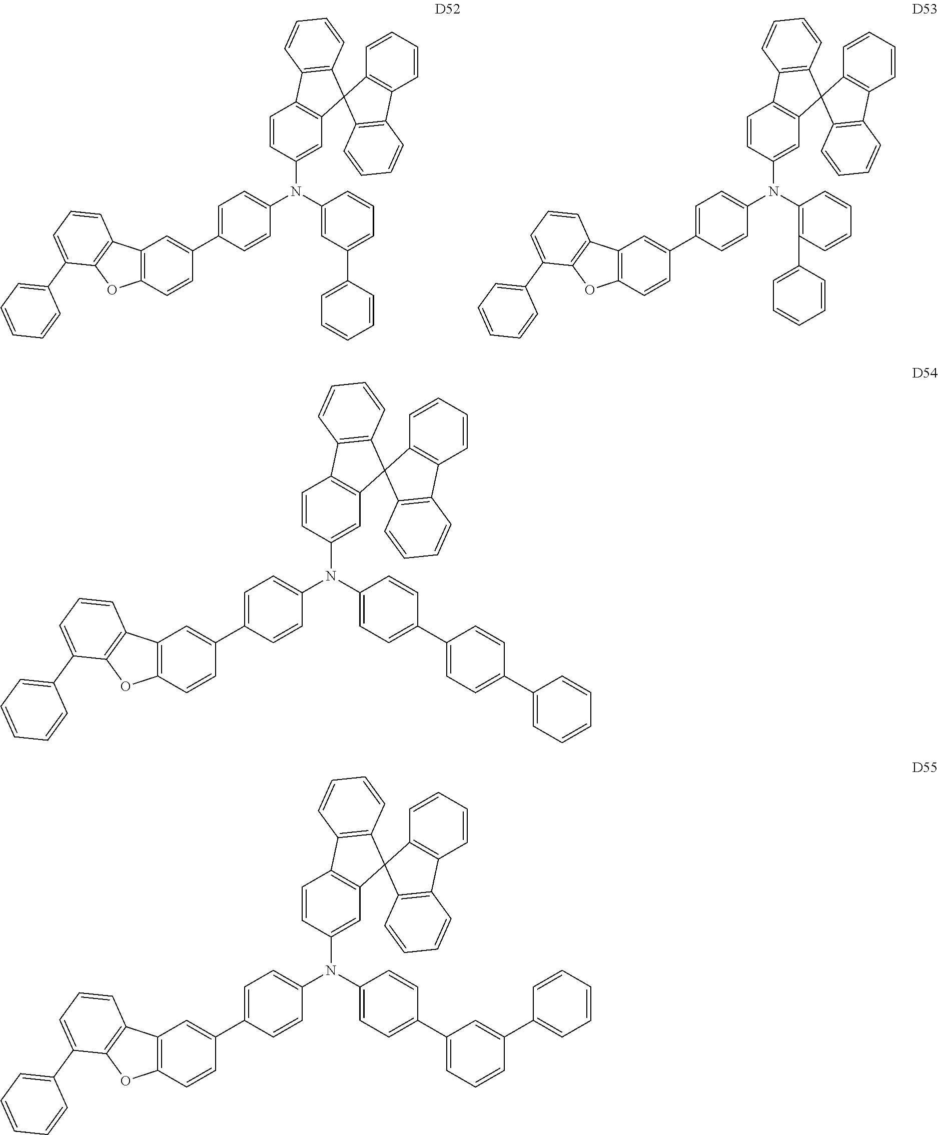

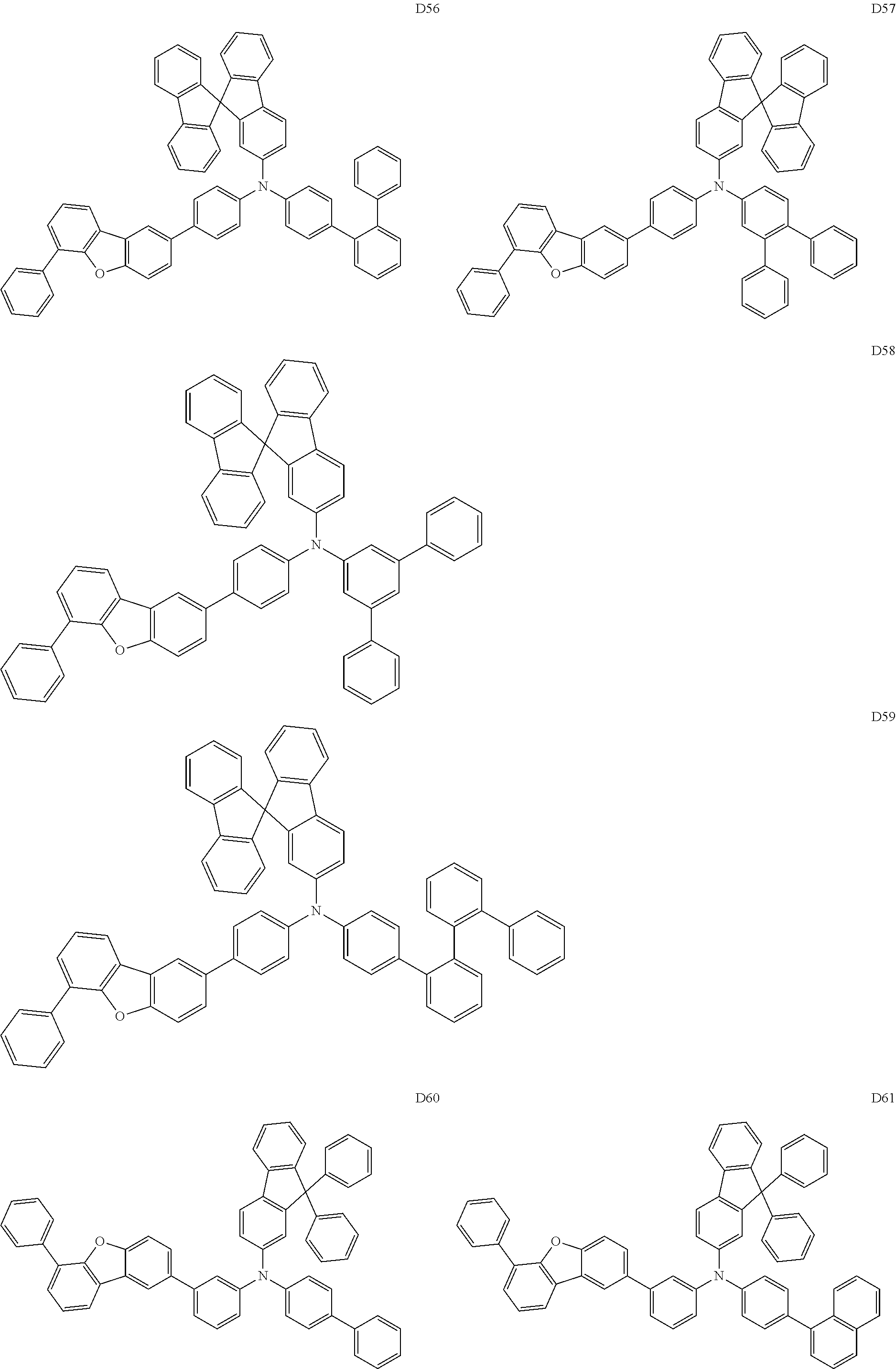

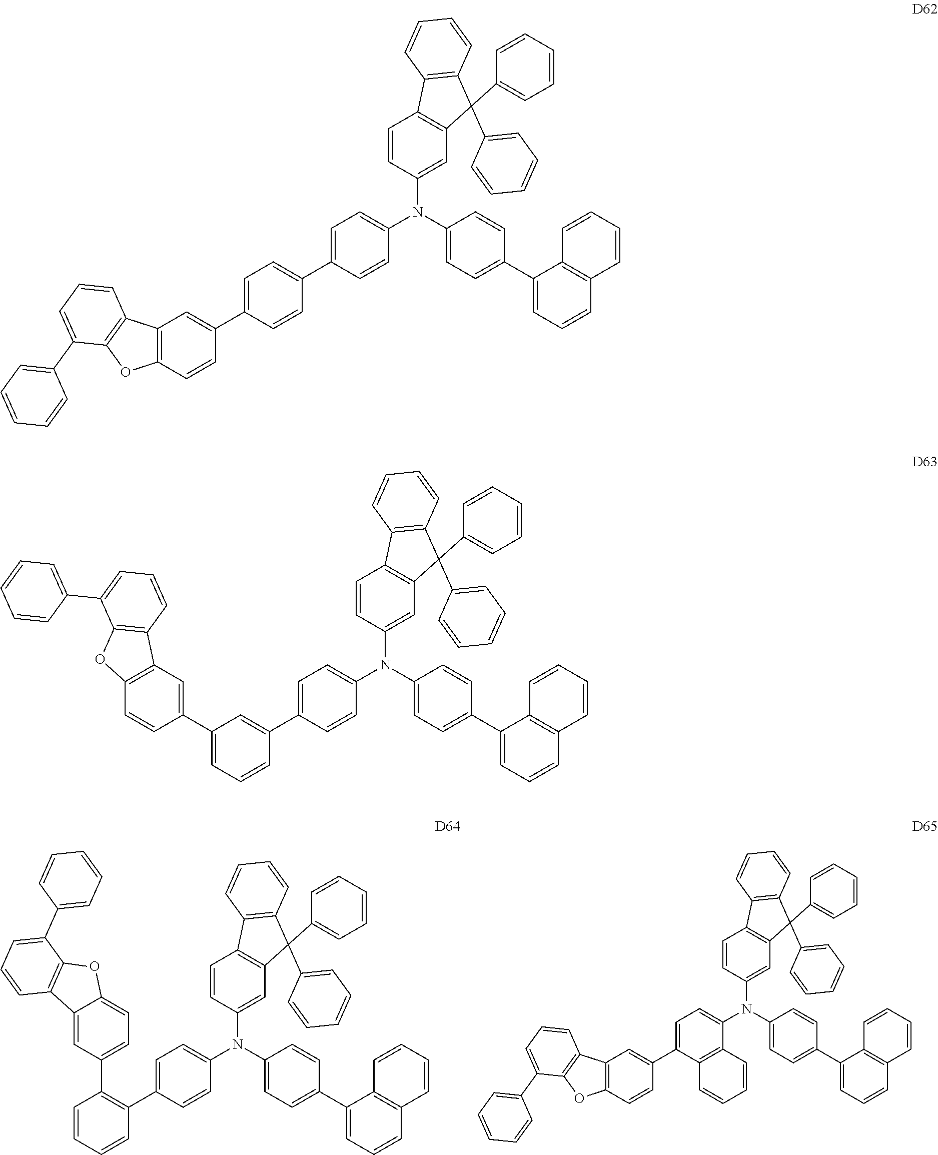 [SMILES: c1ccc(-c2cccc3c2oc2ccc(-c4ccc(-c5ccc(N(c6ccc(-c7cccc8ccccc78)cc6)c6ccc7c(c6)C(c6ccccc6)(c6ccccc6)c6ccccc6-7)cc5)cc4)cc23)cc1 ; c1ccc(-c2cccc3c2oc2ccc(-c4cccc(-c5ccc(N(c6ccc(-c7cccc8ccccc78)cc6)c6ccc7c(c6)C(c6ccccc6)(c6ccccc6)c6ccccc6-7)cc5)c4)cc23)cc1 ; c1ccc(-c2cccc3c2oc2ccc(-c4ccccc4-c4ccc(N(c5ccc(-c6cccc7ccccc67)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6cccc7ccccc67)cc5)c5ccc6c(c5)C(c5ccccc5)(c5ccccc5)c5ccccc5-6)c5ccccc45)cc23)cc1]

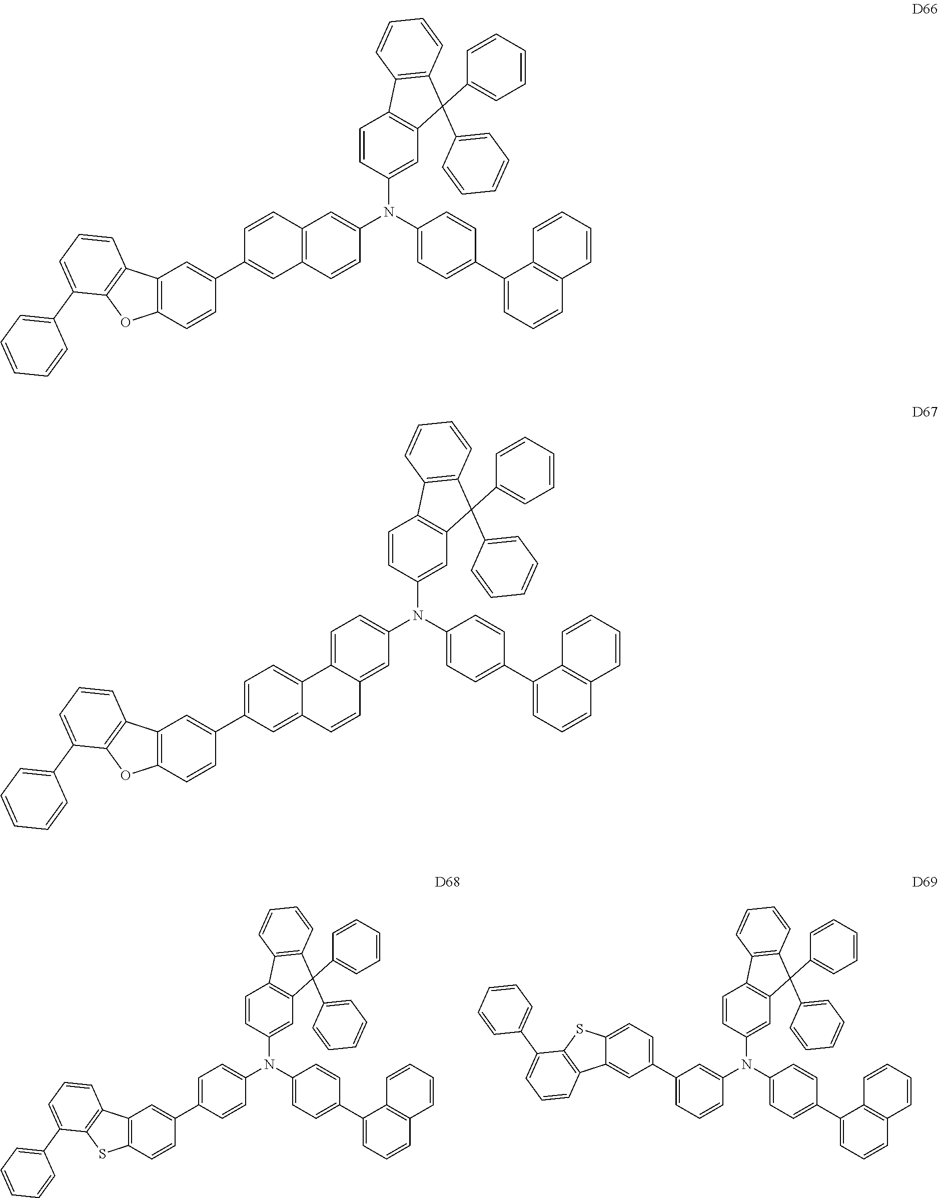 [SMILES: c1ccc(-c2cccc3c2oc2ccc(-c4ccc5cc(N(c6ccc(-c7cccc8ccccc78)cc6)c6ccc7c(c6)C(c6ccccc6)(c6ccccc6)c6ccccc6-7)ccc5c4)cc23)cc1 ; c1ccc(-c2cccc3c2oc2ccc(-c4ccc5c(ccc6cc(N(c7ccc(-c8cccc9ccccc89)cc7)c7ccc8c(c7)C(c7ccccc7)(c7ccccc7)c7ccccc7-8)ccc65)c4)cc23)cc1 ; c1ccc(-c2cccc3c2sc2ccc(-c4ccc(N(c5ccc(-c6cccc7ccccc67)cc5)c5ccc6c(c5)C(c5ccccc5)(c5ccccc5)c5ccccc5-6)cc4)cc23)cc1 ; c1ccc(-c2cccc3c2sc2ccc(-c4cccc(N(c5ccc(-c6cccc7ccccc67)cc5)c5ccc6c(c5)C(c5ccccc5)(c5ccccc5)c5ccccc5-6)c4)cc23)cc1]

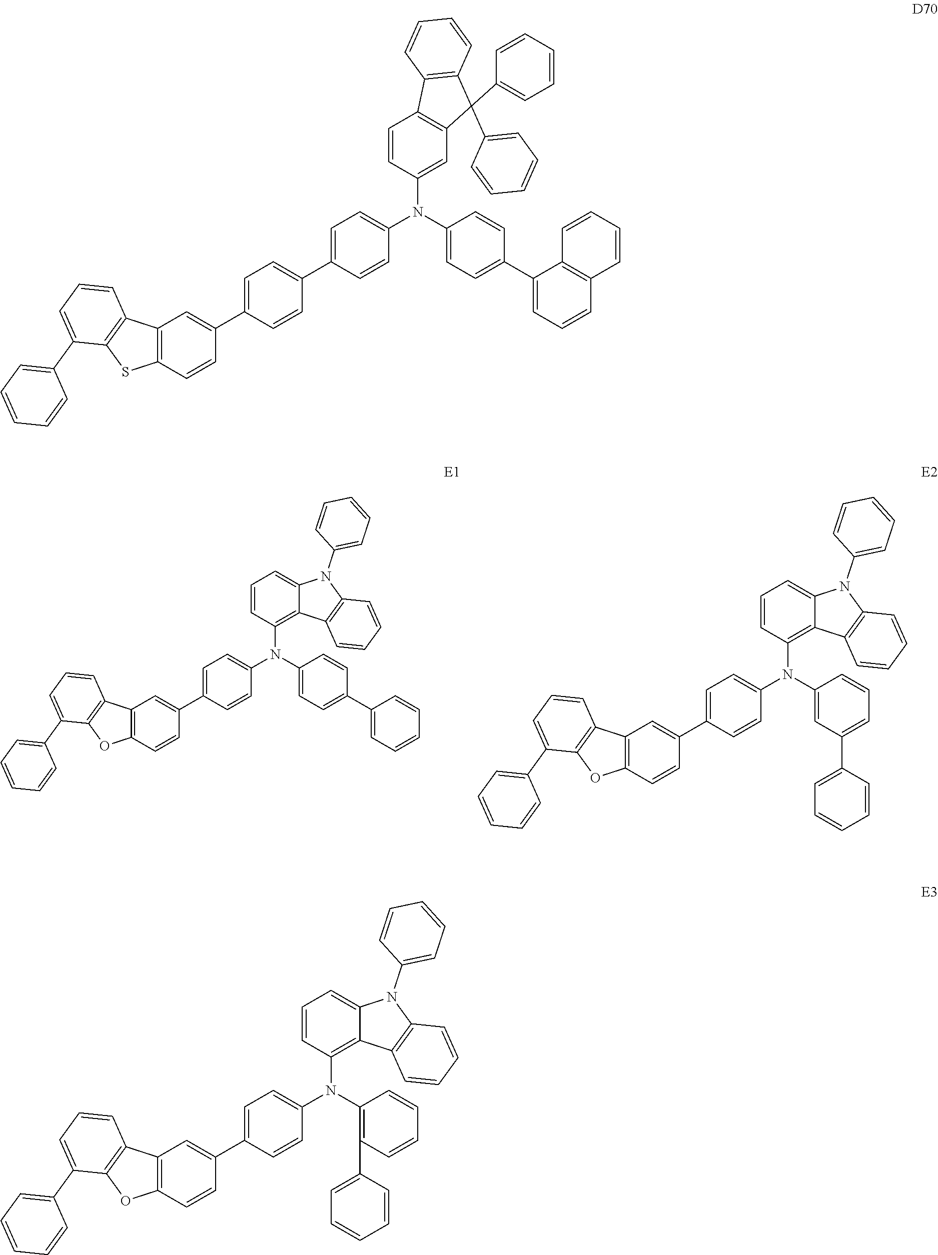 [SMILES: c1ccc(-c2cccc3c2sc2ccc(-c4ccc(-c5ccc(N(c6ccc(-c7cccc8ccccc78)cc6)c6ccc7c(c6)C(c6ccccc6)(c6ccccc6)c6ccccc6-7)cc5)cc4)cc23)cc1 ; c1ccc(-c2ccc(N(c3ccc(-c4ccc5oc6c(-c7ccccc7)cccc6c5c4)cc3)c3cccc4c3c3ccccc3n4-c3ccccc3)cc2)cc1 ; c1ccc(-c2cccc(N(c3ccc(-c4ccc5oc6c(-c7ccccc7)cccc6c5c4)cc3)c3cccc4c3c3ccccc3n4-c3ccccc3)c2)cc1 ; c1ccc(-c2ccccc2N(c2ccc(-c3ccc4oc5c(-c6ccccc6)cccc5c4c3)cc2)c2cccc3c2c2ccccc2n3-c2ccccc2)cc1]

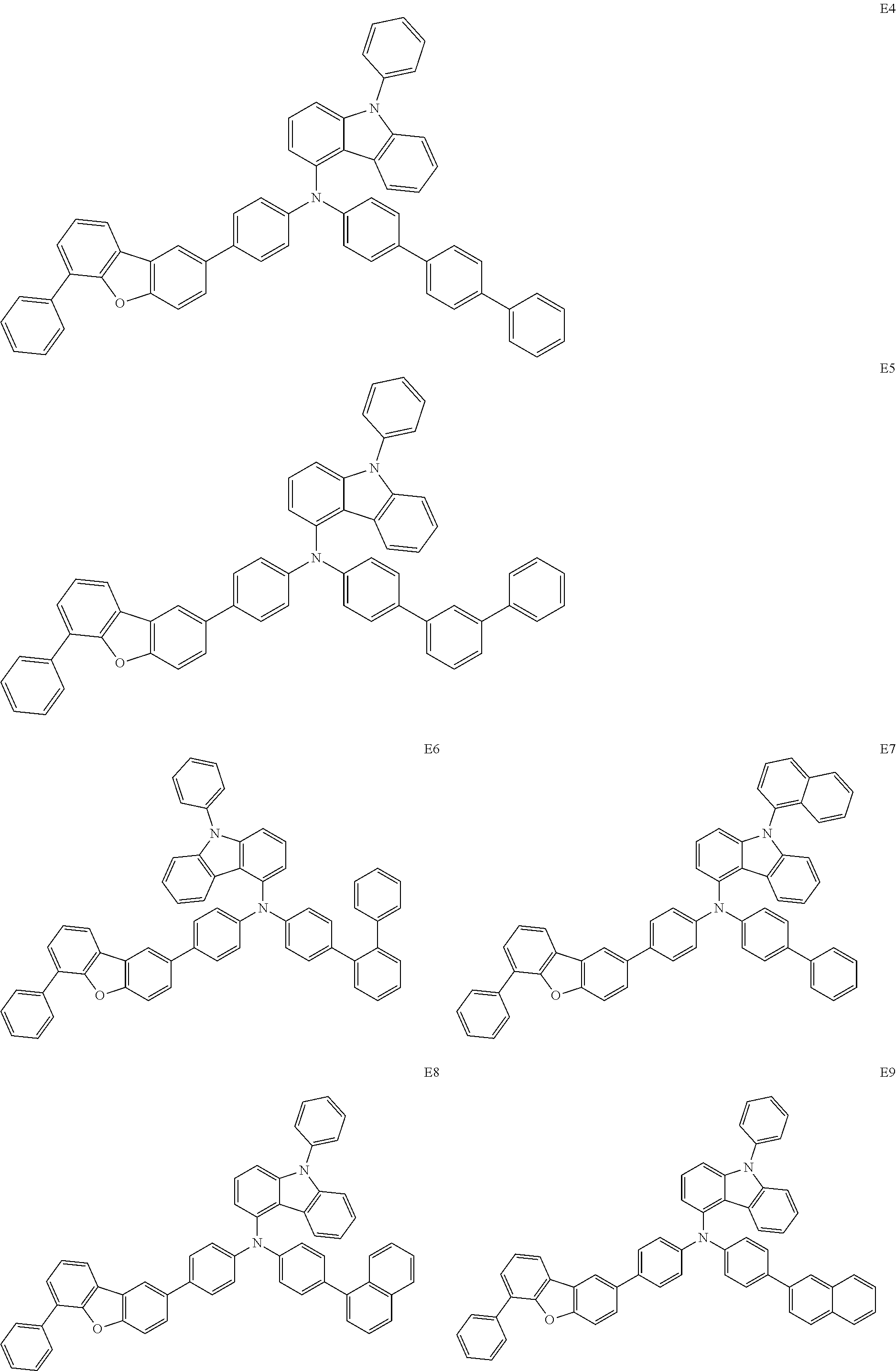

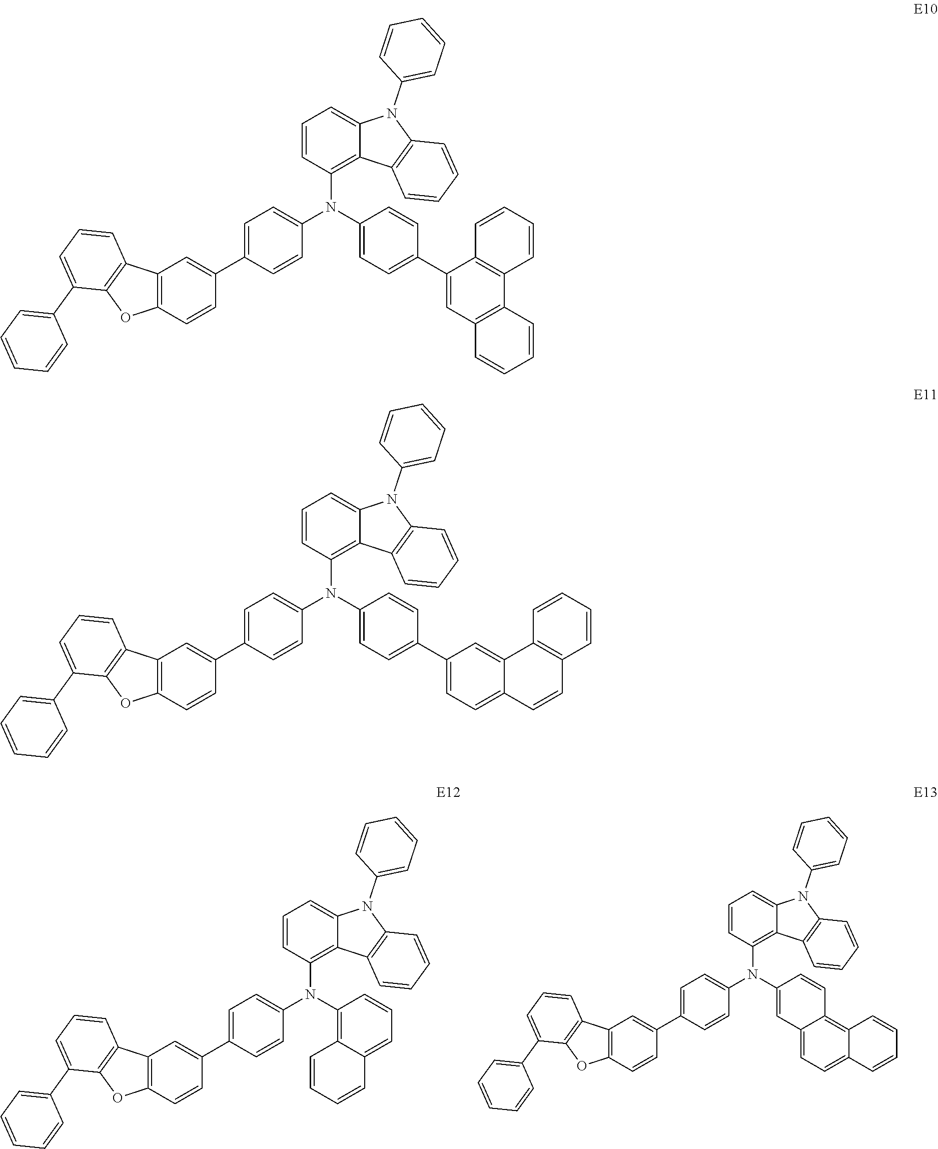 [SMILES: c1ccc(-c2cccc3c2oc2ccc(-c4ccc(N(c5ccc(-c6cc7ccccc7c7ccccc67)cc5)c5cccc6c5c5ccccc5n6-c5ccccc5)cc4)cc23)cc1 ; c1ccc(-c2cccc3c2oc2ccc(-c4ccc(N(c5ccc(-c6ccc7ccc8ccccc8c7c6)cc5)c5cccc6c5c5ccccc5n6-c5ccccc5)cc4)cc23)cc1 ; c1ccc(-c2cccc3c2oc2ccc(-c4ccc(N(c5cccc6ccccc56)c5cccc6c5c5ccccc5n6-c5ccccc5)cc4)cc23)cc1 ; c1ccc(-c2cccc3c2oc2ccc(-c4ccc(N(c5ccc6c(ccc7ccccc76)c5)c5cccc6c5c5ccccc5n6-c5ccccc5)cc4)cc23)cc1]

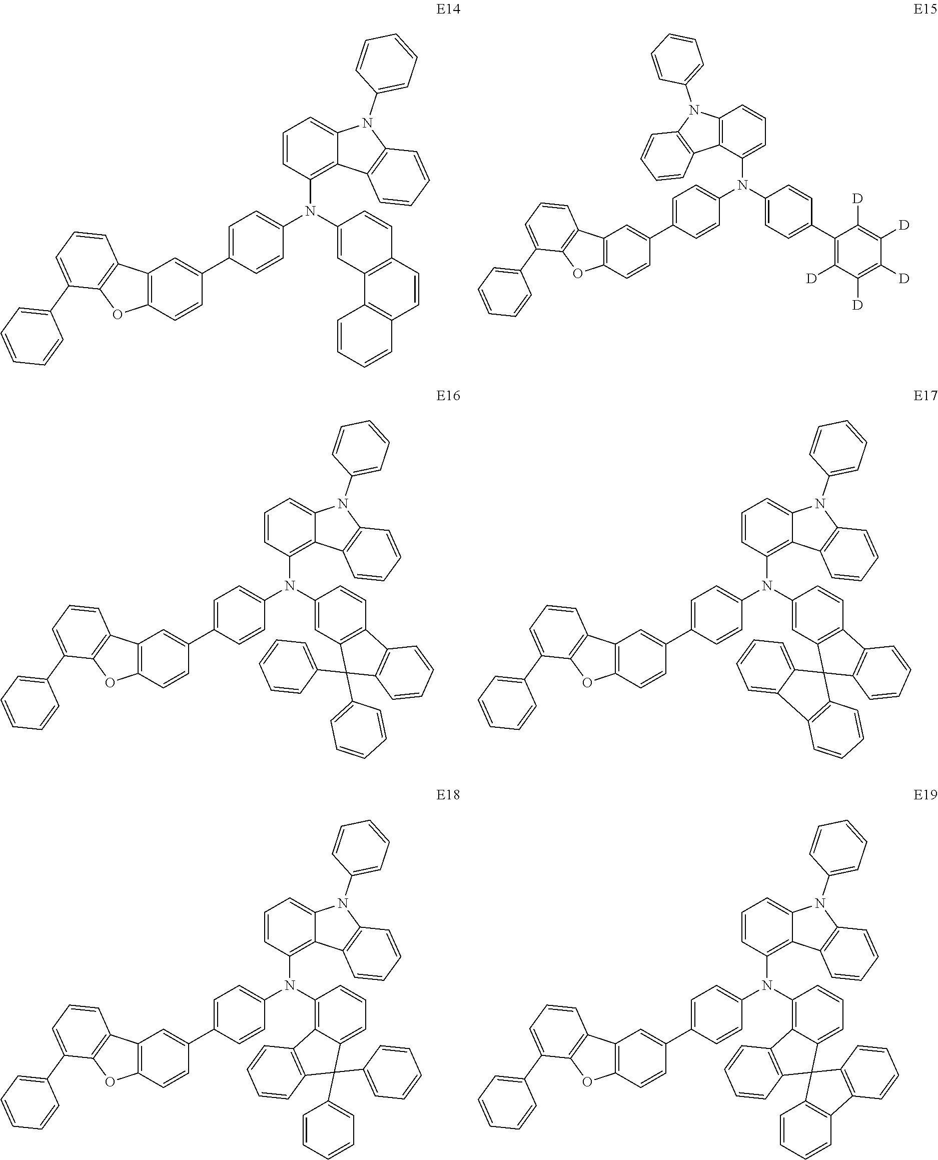

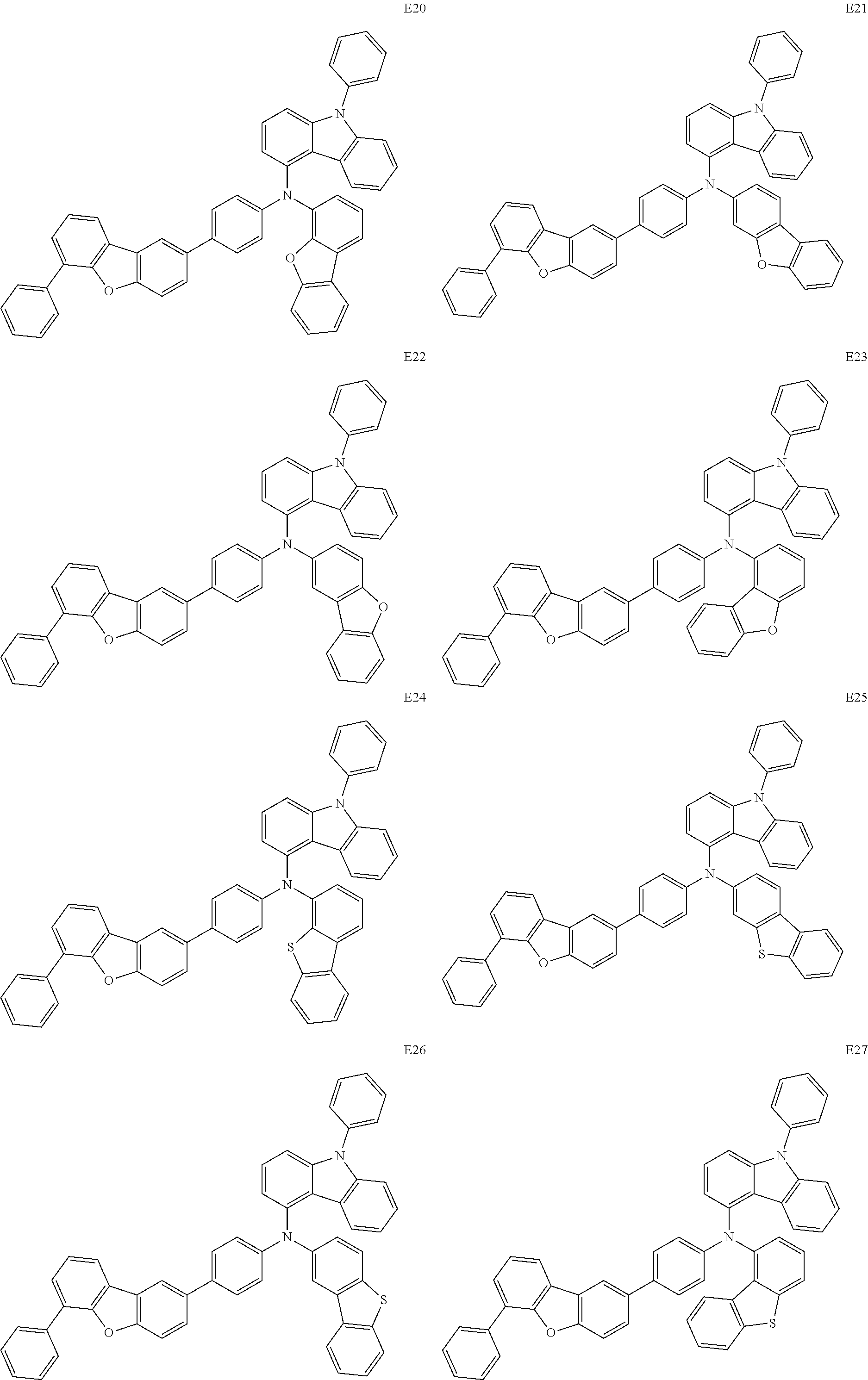

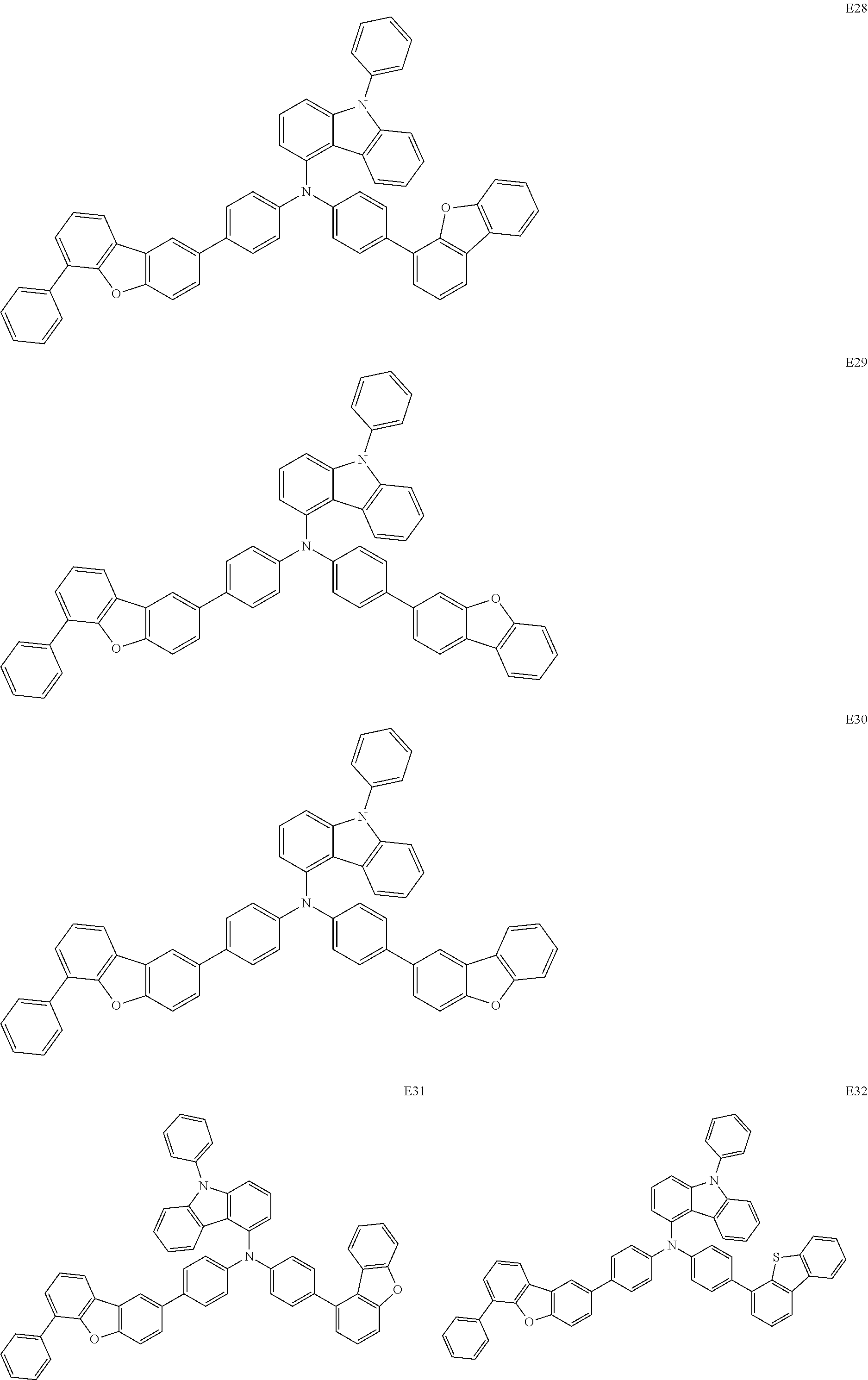

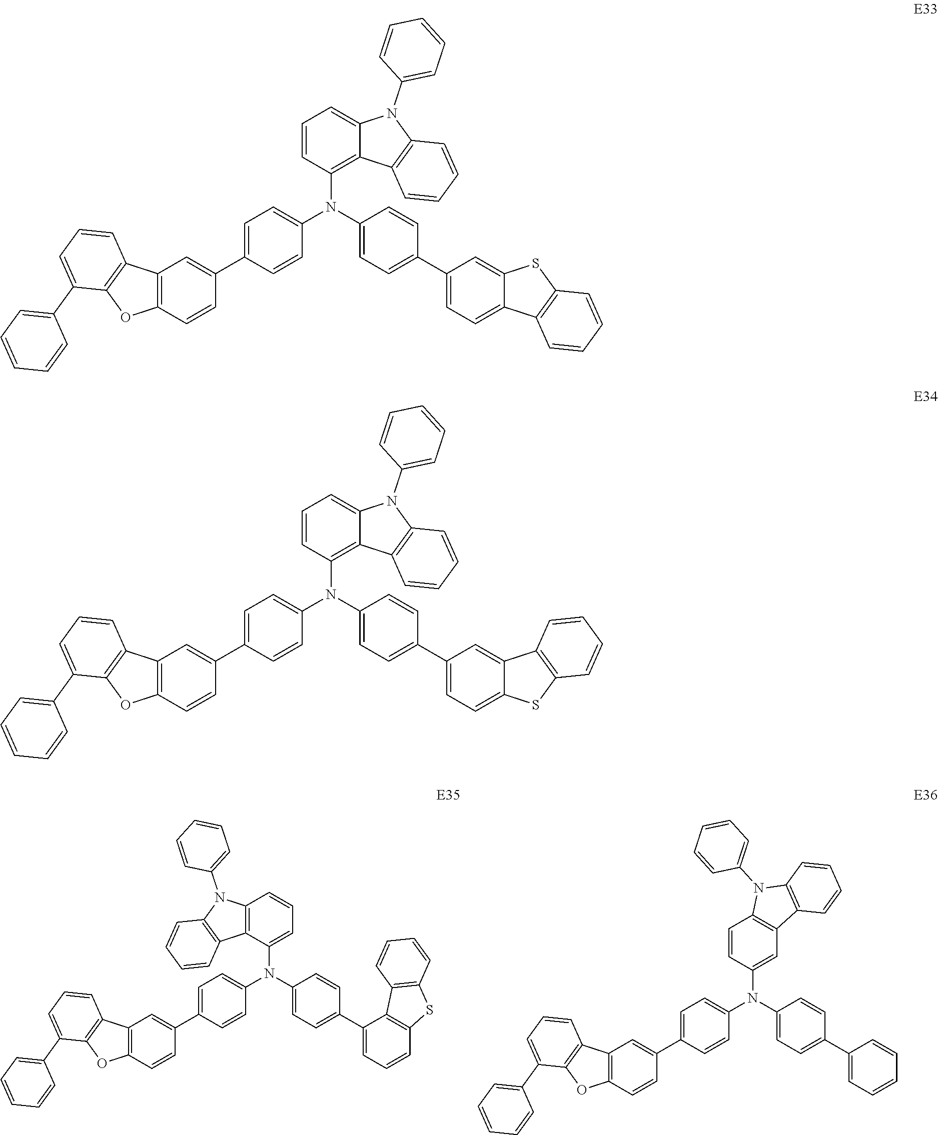 [SMILES: c1ccc(-c2cccc3c2oc2ccc(-c4ccc(N(c5ccc(-c6ccc7c(c6)sc6ccccc67)cc5)c5cccc6c5c5ccccc5n6-c5ccccc5)cc4)cc23)cc1 ; c1ccc(-c2cccc3c2oc2ccc(-c4ccc(N(c5ccc(-c6ccc7sc8ccccc8c7c6)cc5)c5cccc6c5c5ccccc5n6-c5ccccc5)cc4)cc23)cc1 ; c1ccc(-c2cccc3c2oc2ccc(-c4ccc(N(c5ccc(-c6cccc7sc8ccccc8c67)cc5)c5cccc6c5c5ccccc5n6-c5ccccc5)cc4)cc23)cc1 ; c1ccc(-c2ccc(N(c3ccc(-c4ccc5oc6c(-c7ccccc7)cccc6c5c4)cc3)c3ccc4c(c3)c3ccccc3n4-c3ccccc3)cc2)cc1]

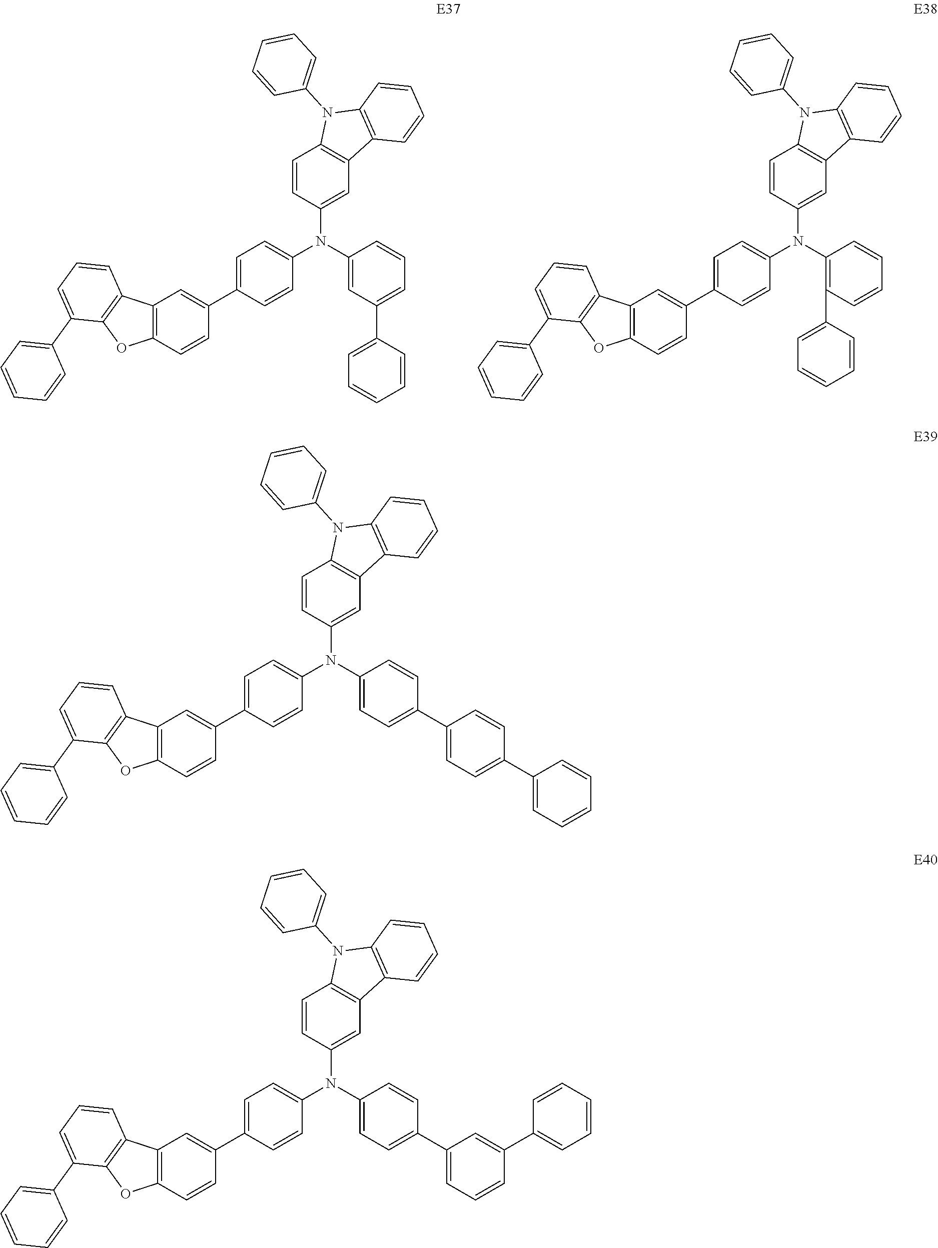 [SMILES: c1ccc(-c2cccc(N(c3ccc(-c4ccc5oc6c(-c7ccccc7)cccc6c5c4)cc3)c3ccc4c(c3)c3ccccc3n4-c3ccccc3)c2)cc1 ; c1ccc(-c2ccccc2N(c2ccc(-c3ccc4oc5c(-c6ccccc6)cccc5c4c3)cc2)c2ccc3c(c2)c2ccccc2n3-c2ccccc2)cc1 ; c1ccc(-c2ccc(-c3ccc(N(c4ccc(-c5ccc6oc7c(-c8ccccc8)cccc7c6c5)cc4)c4ccc5c(c4)c4ccccc4n5-c4ccccc4)cc3)cc2)cc1 ; c1ccc(-c2cccc(-c3ccc(N(c4ccc(-c5ccc6oc7c(-c8ccccc8)cccc7c6c5)cc4)c4ccc5c(c4)c4ccccc4n5-c4ccccc4)cc3)c2)cc1]

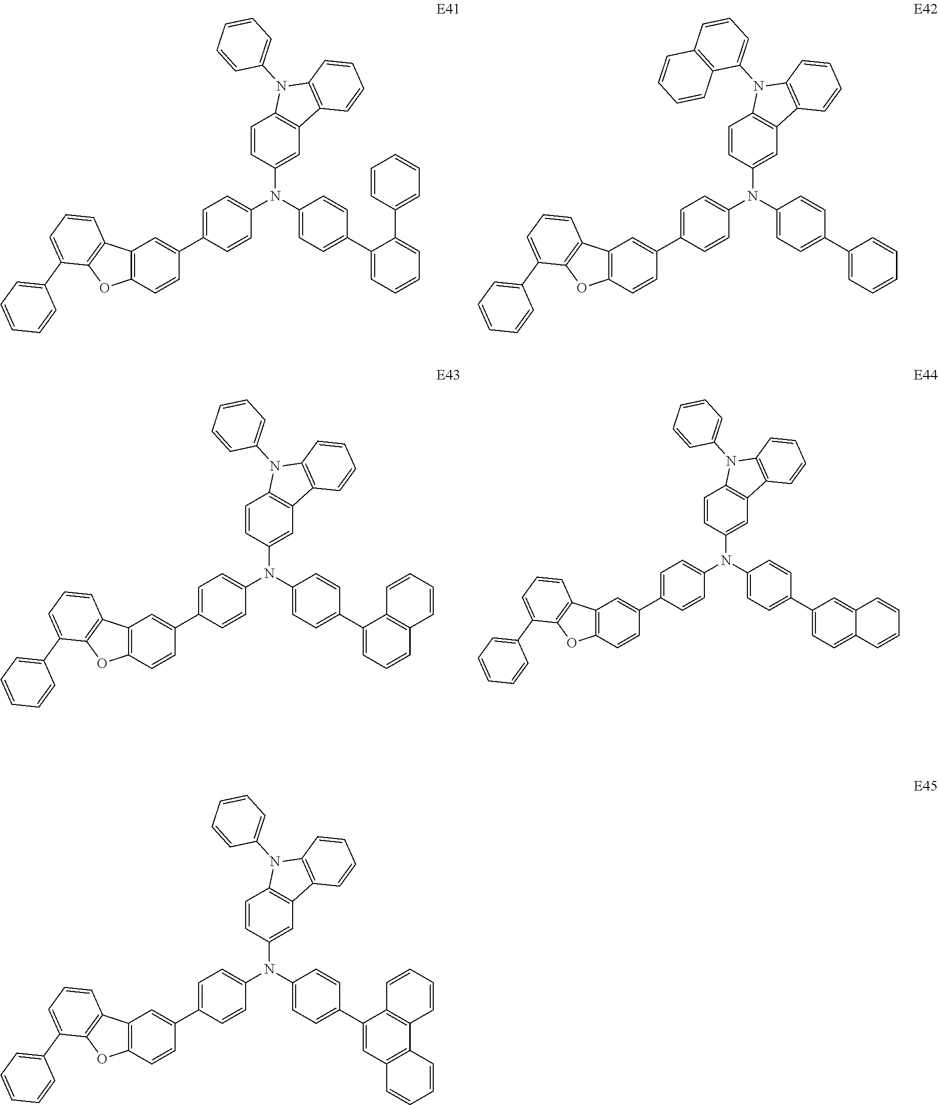

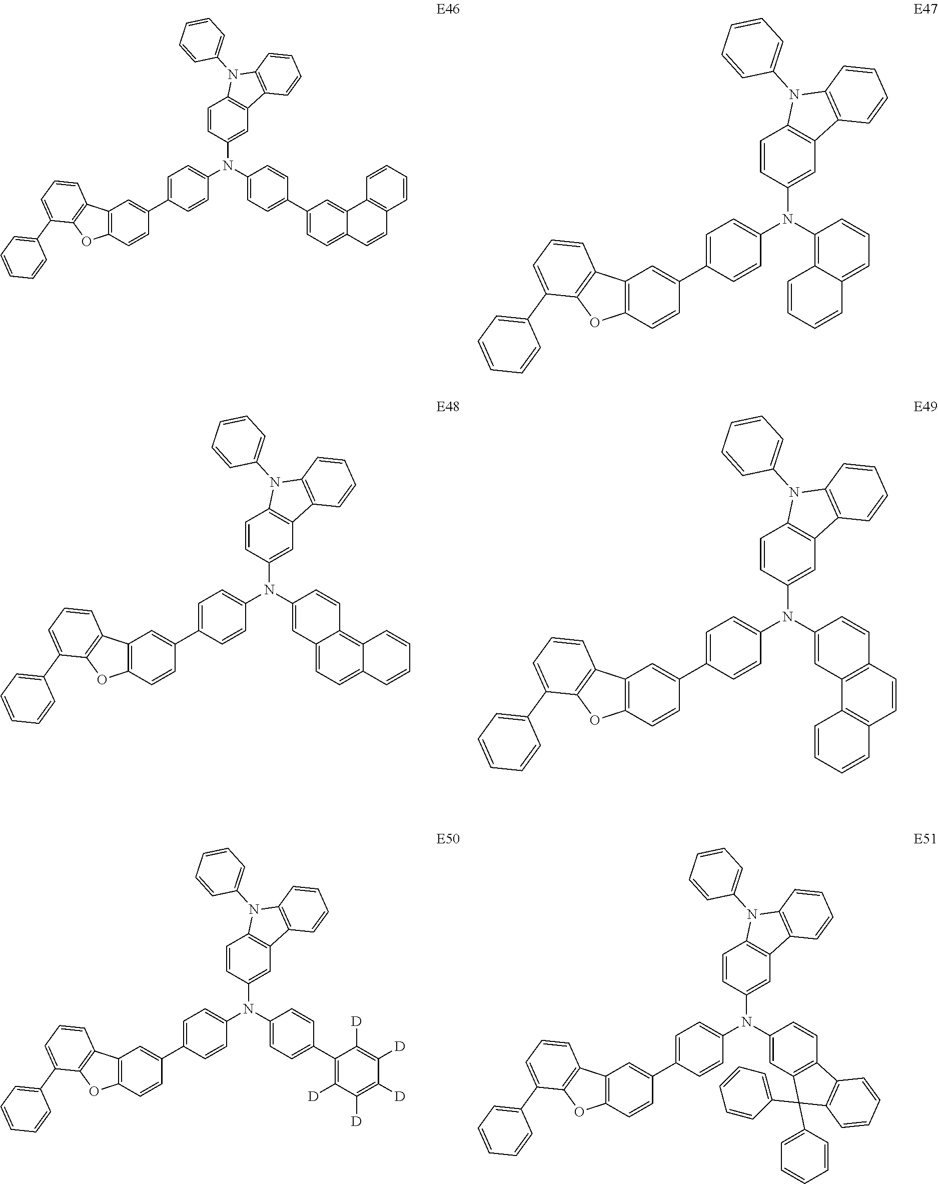

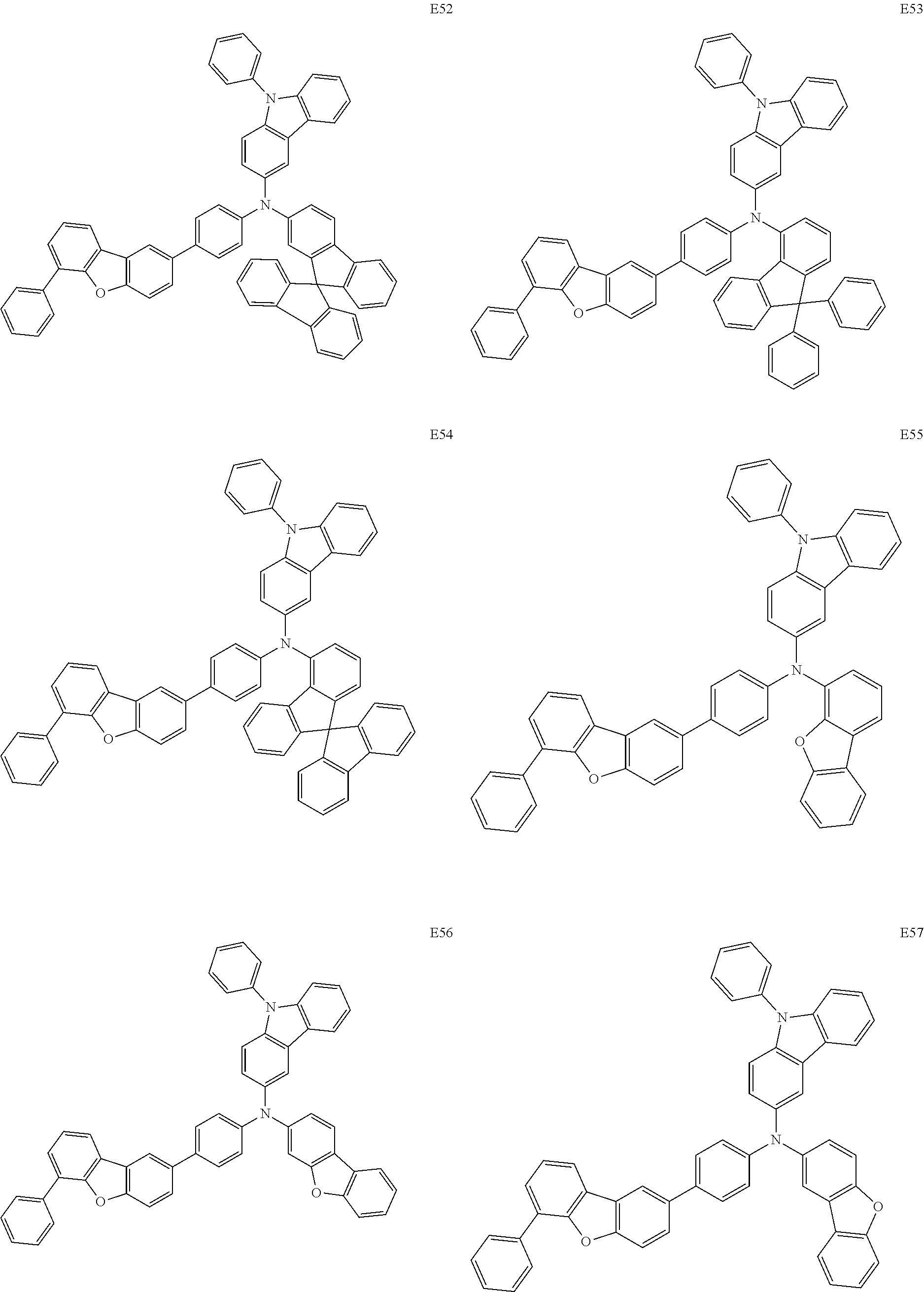

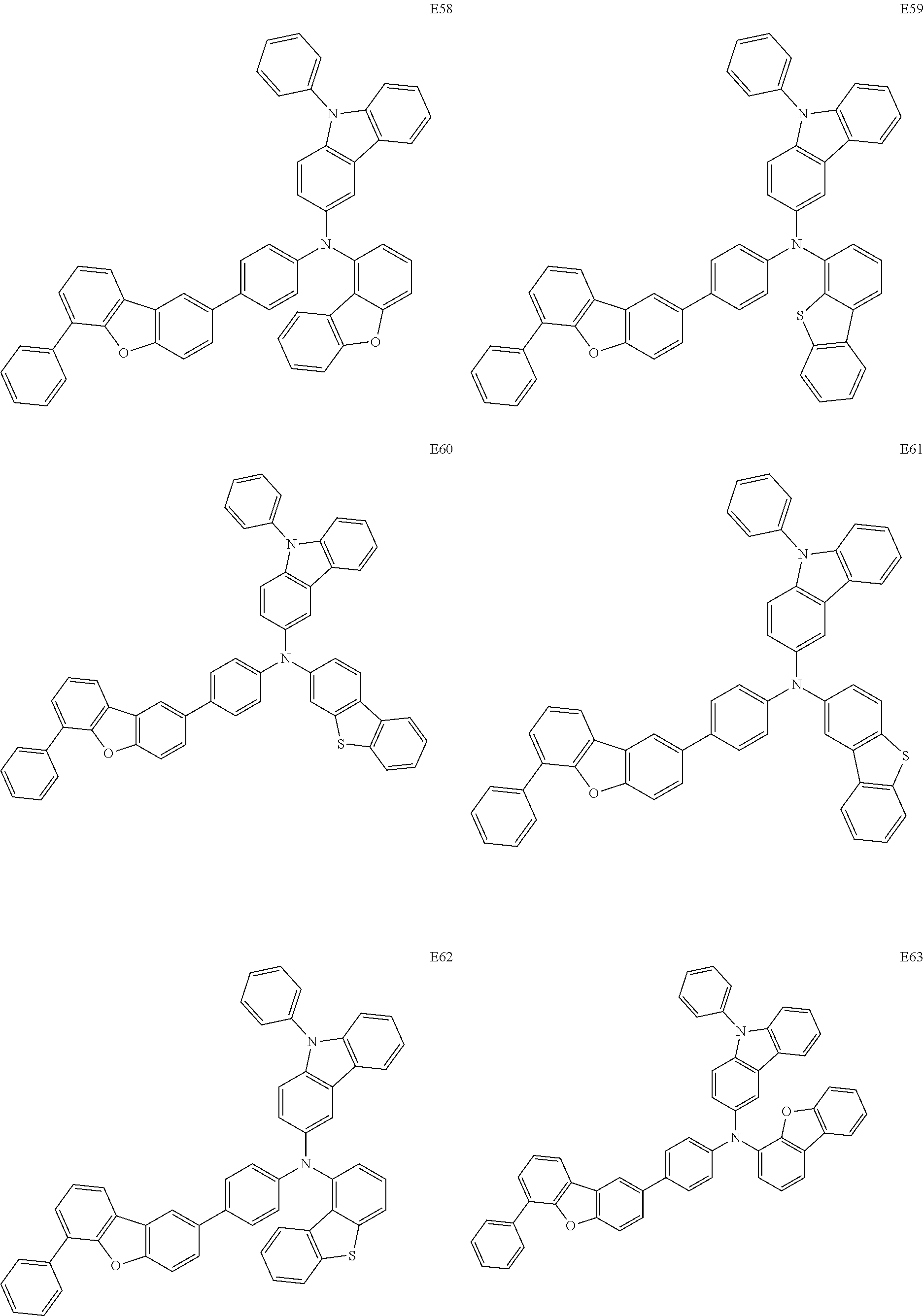

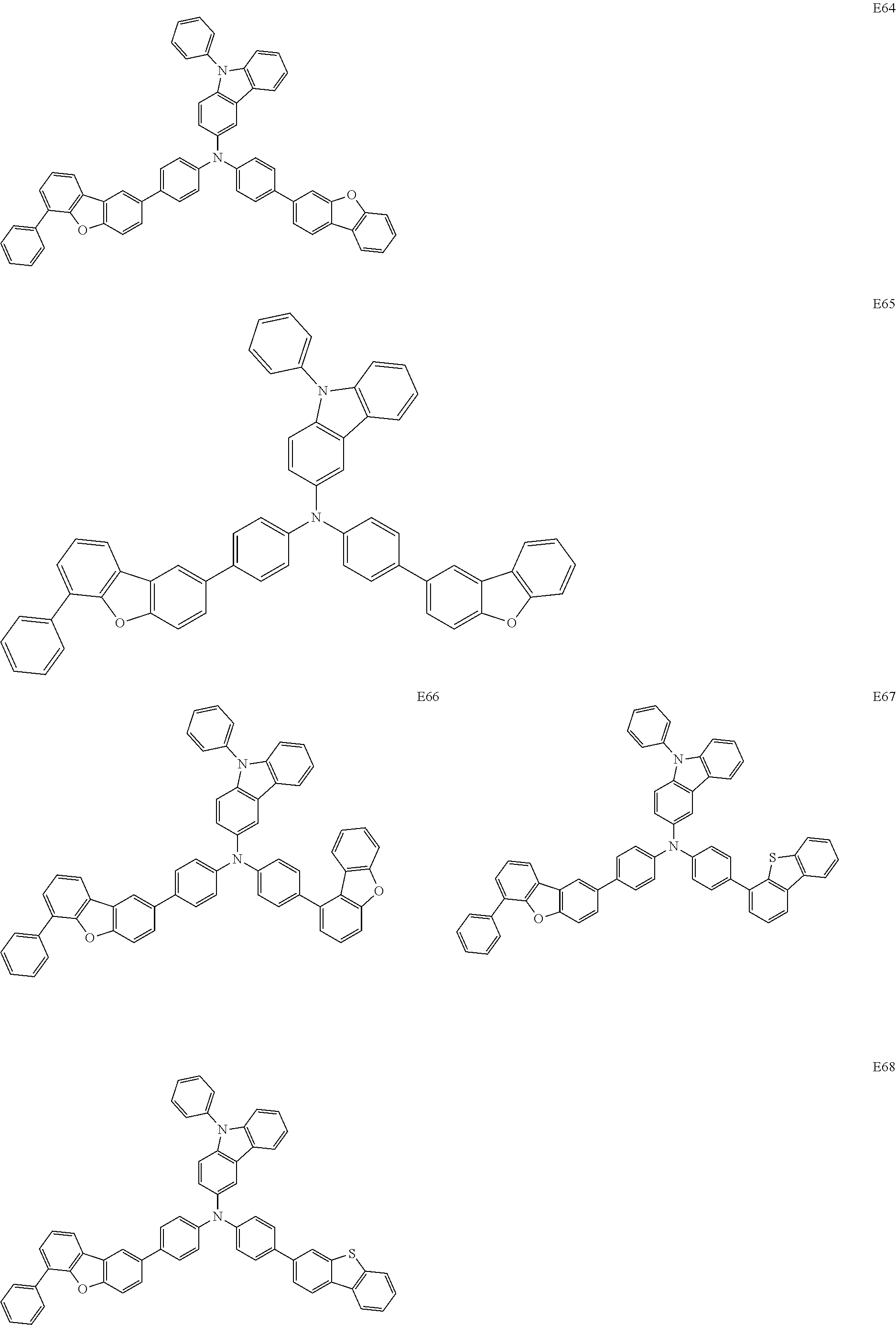

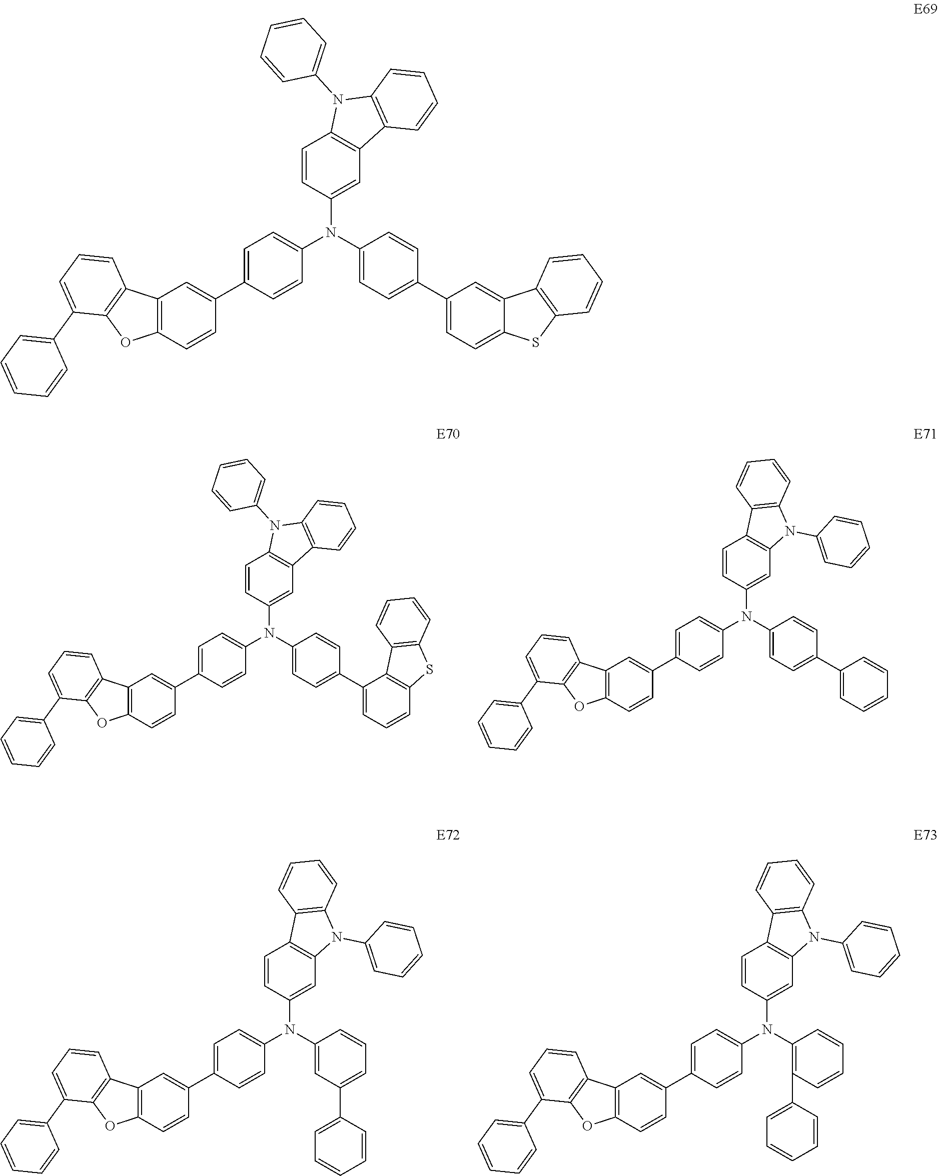

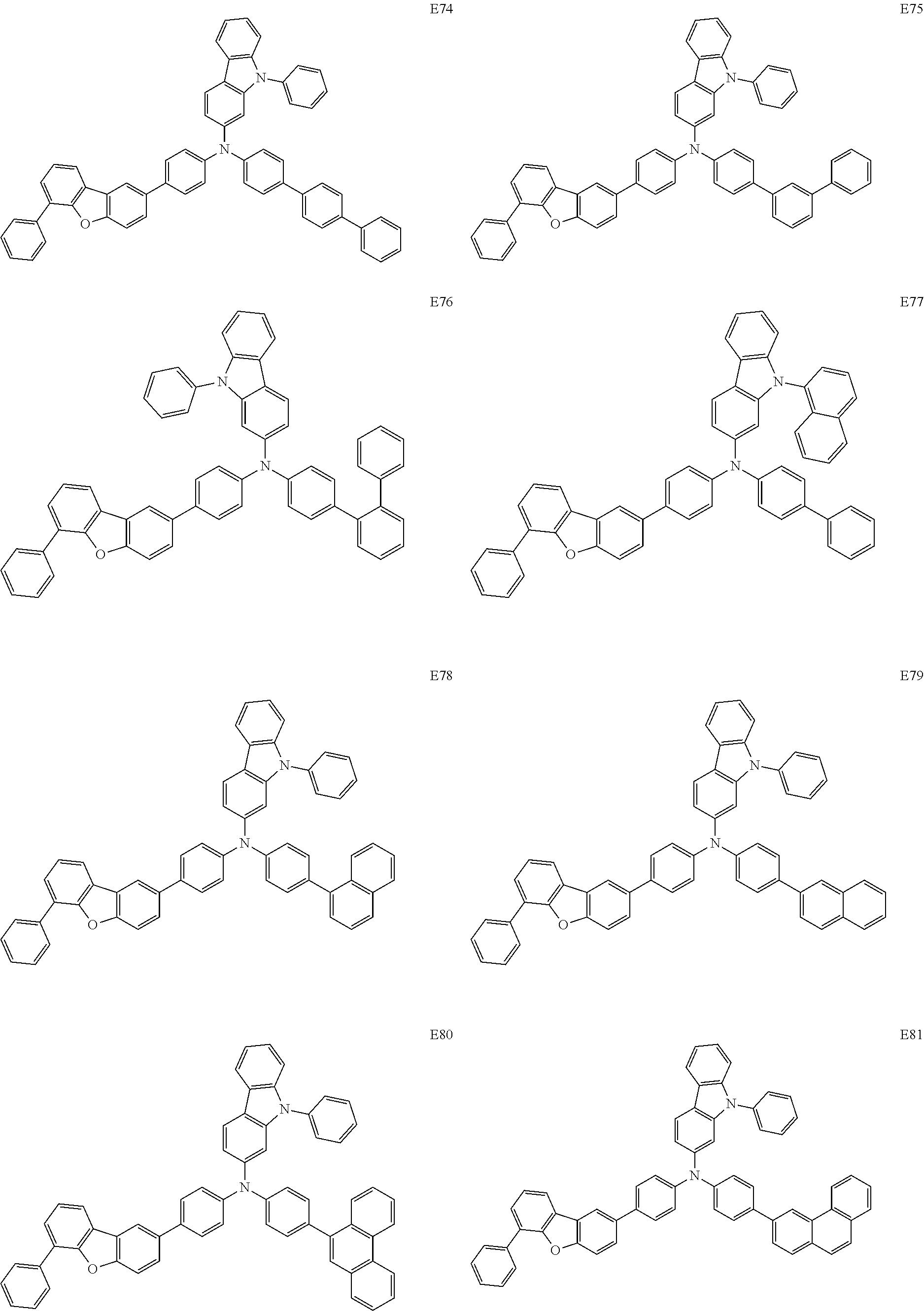

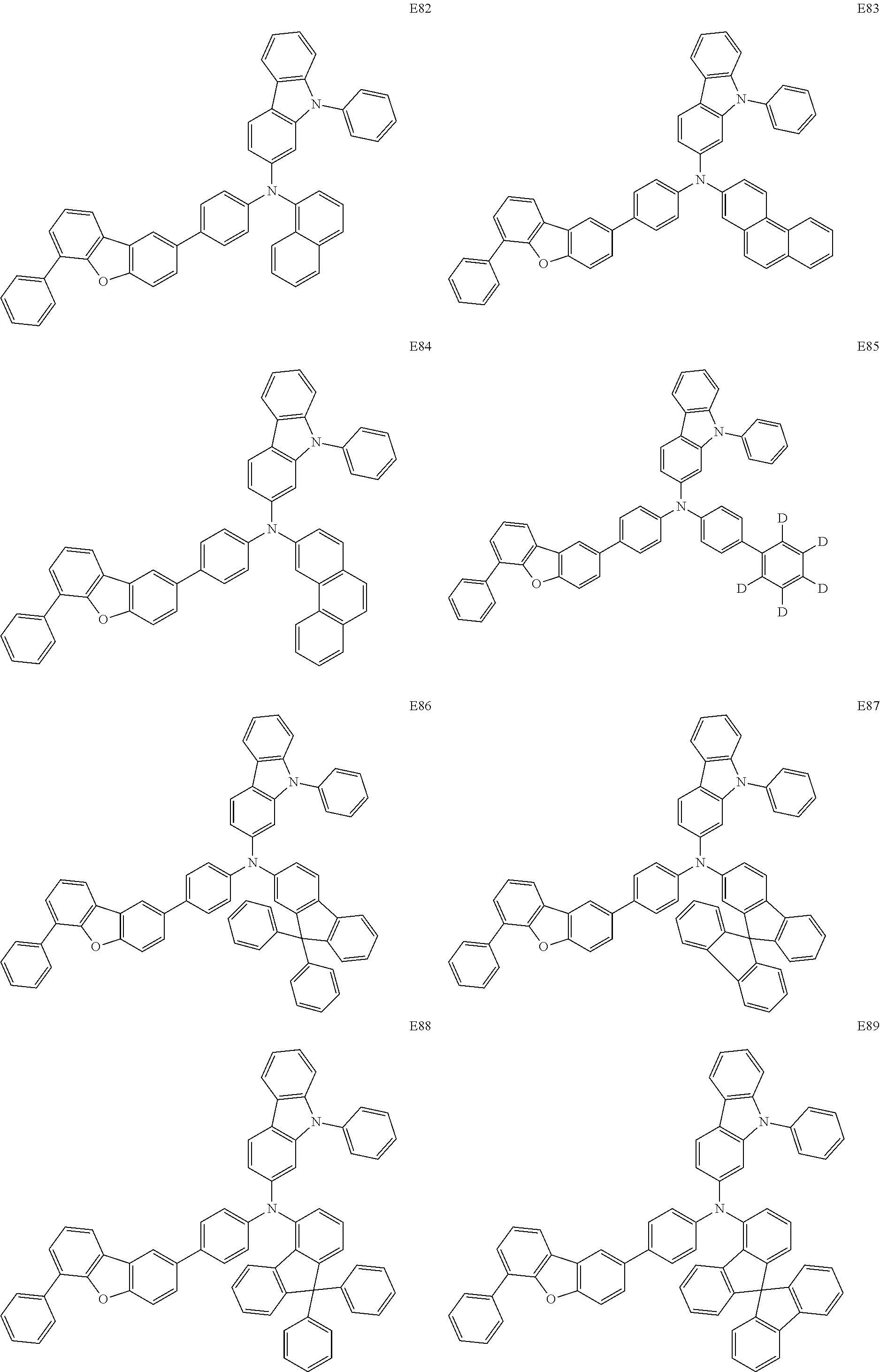

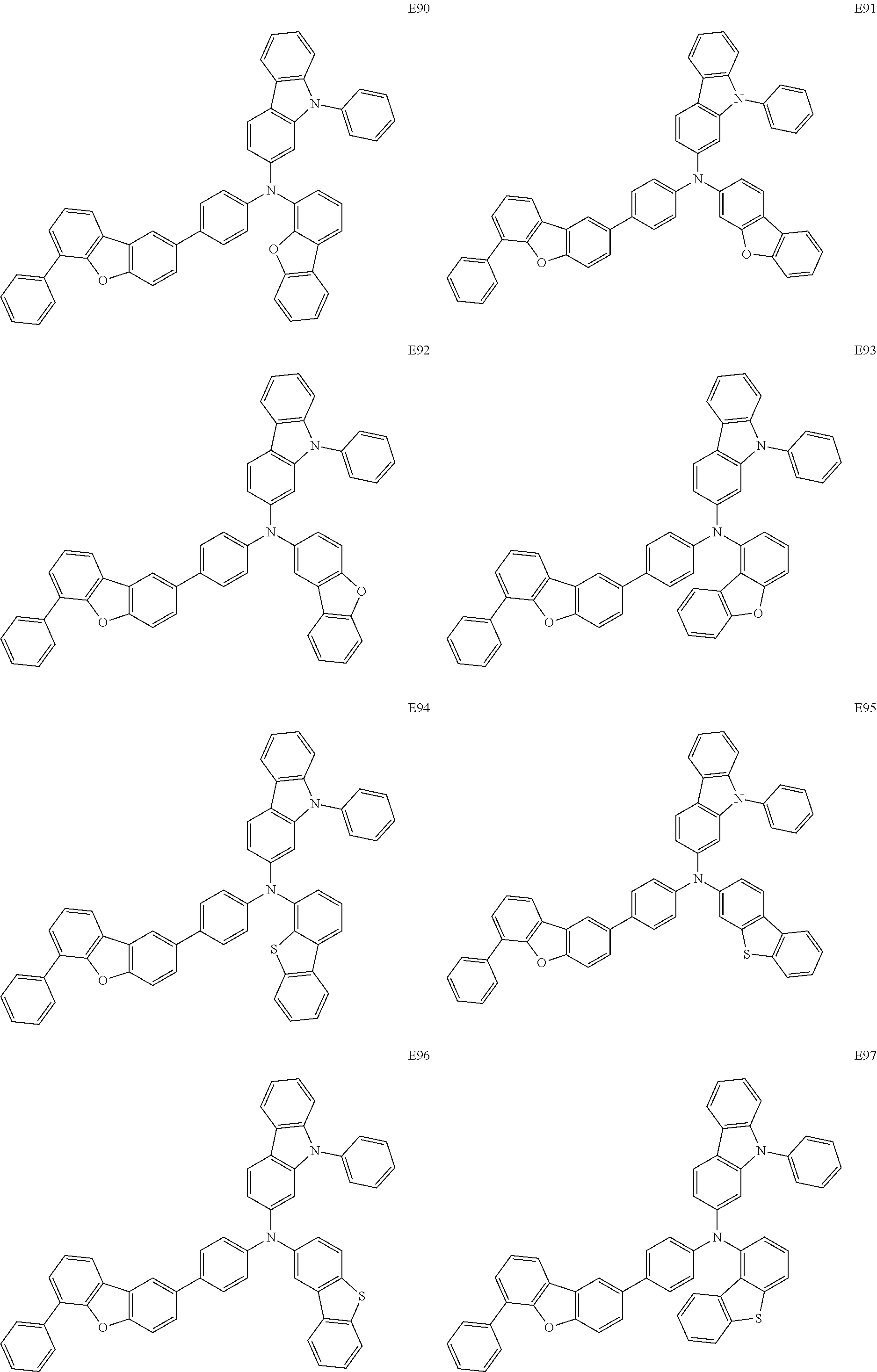

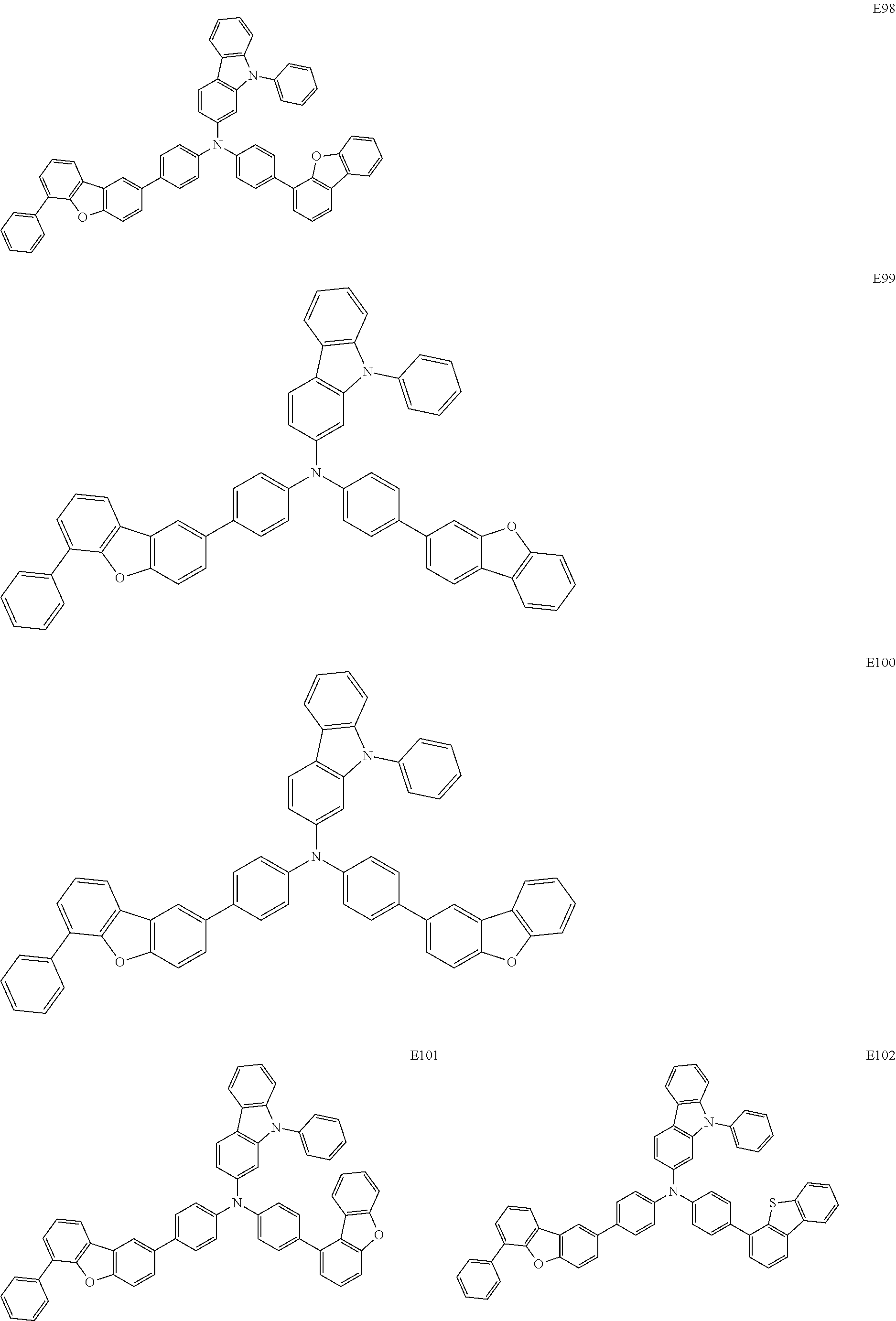

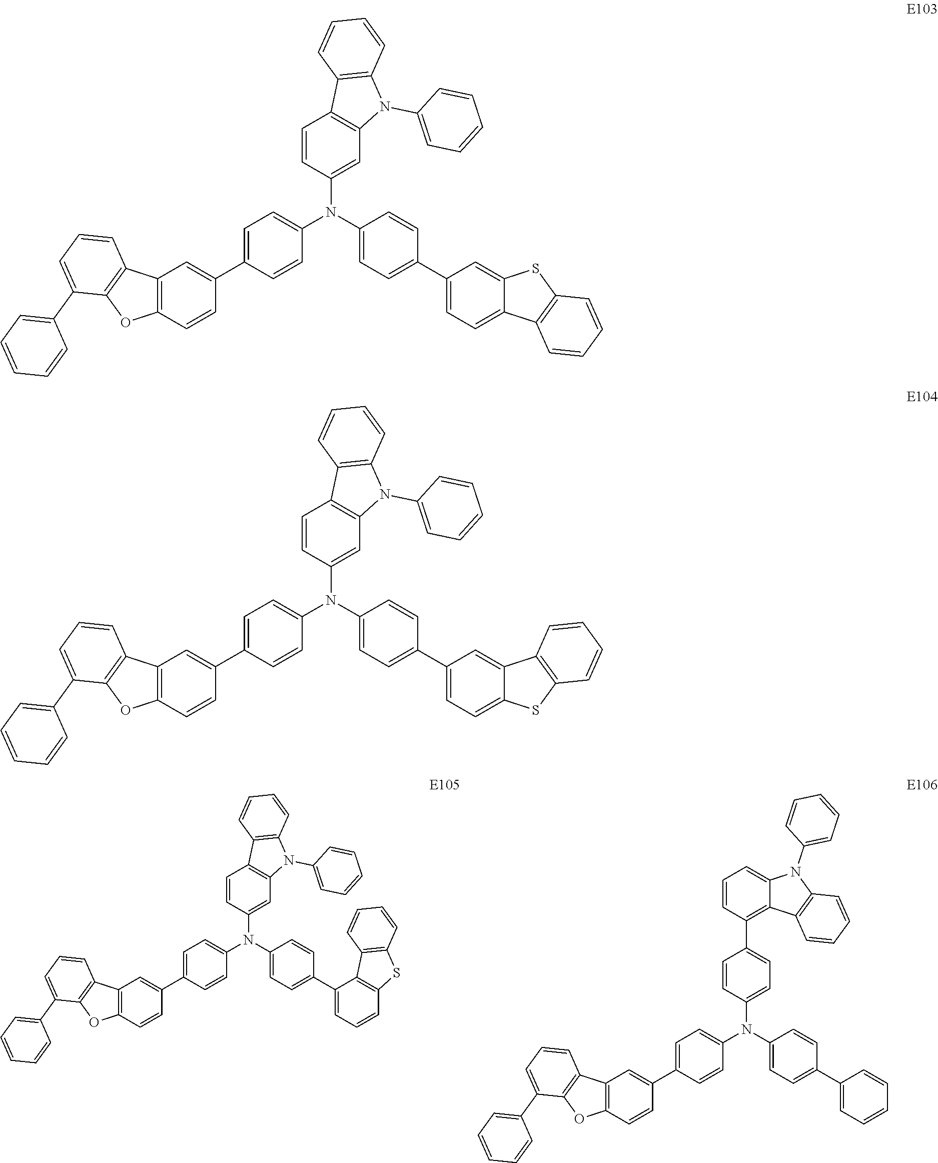 [SMILES: c1ccc(-c2cccc3c2oc2ccc(-c4ccc(N(c5ccc(-c6ccc7c(c6)sc6ccccc67)cc5)c5ccc6c7ccccc7n(-c7ccccc7)c6c5)cc4)cc23)cc1 ; c1ccc(-c2cccc3c2oc2ccc(-c4ccc(N(c5ccc(-c6ccc7sc8ccccc8c7c6)cc5)c5ccc6c7ccccc7n(-c7ccccc7)c6c5)cc4)cc23)cc1 ; c1ccc(-c2cccc3c2oc2ccc(-c4ccc(N(c5ccc(-c6cccc7sc8ccccc8c67)cc5)c5ccc6c7ccccc7n(-c7ccccc7)c6c5)cc4)cc23)cc1 ; c1ccc(-c2ccc(N(c3ccc(-c4ccc5oc6c(-c7ccccc7)cccc6c5c4)cc3)c3ccc(-c4cccc5c4c4ccccc4n5-c4ccccc4)cc3)cc2)cc1]

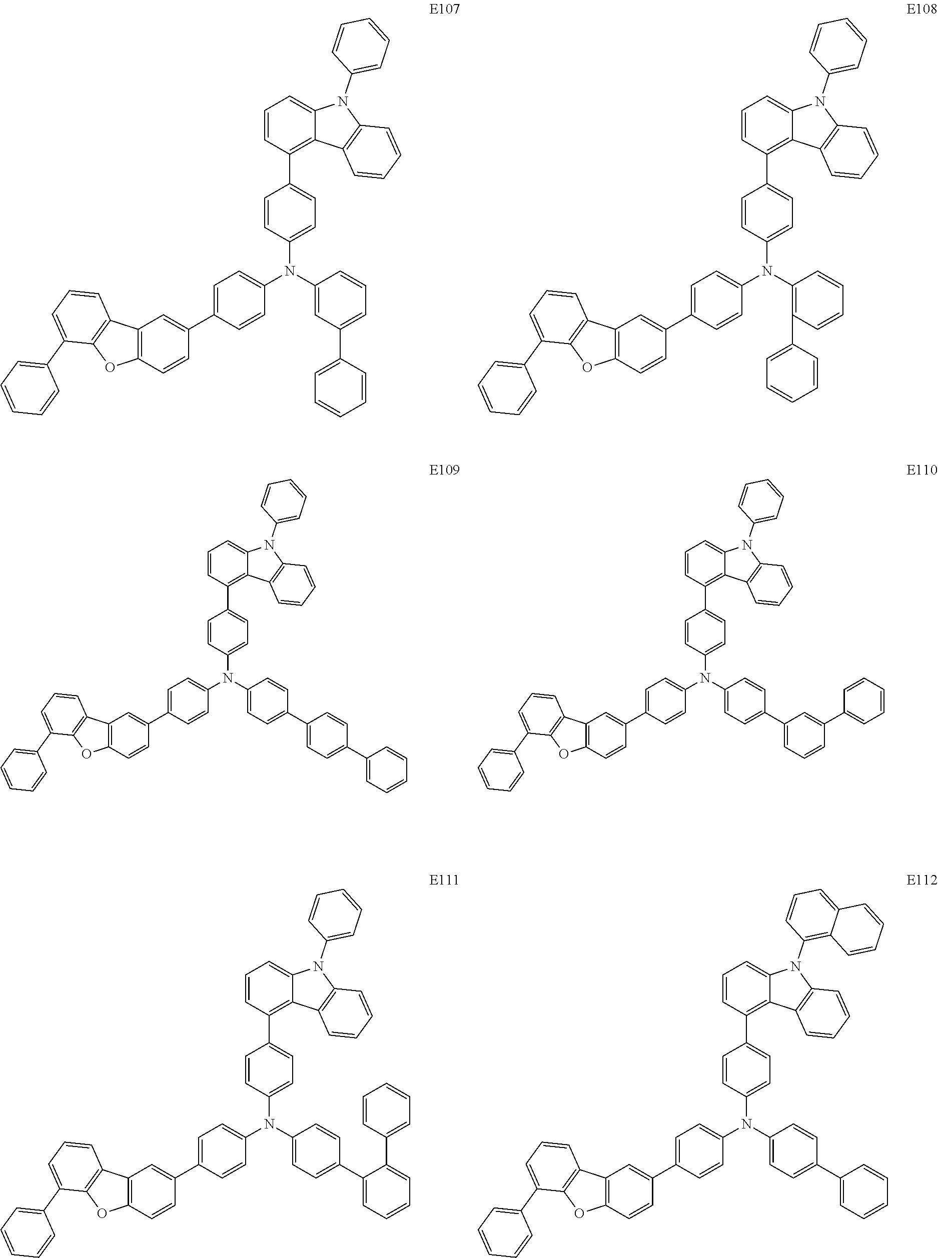

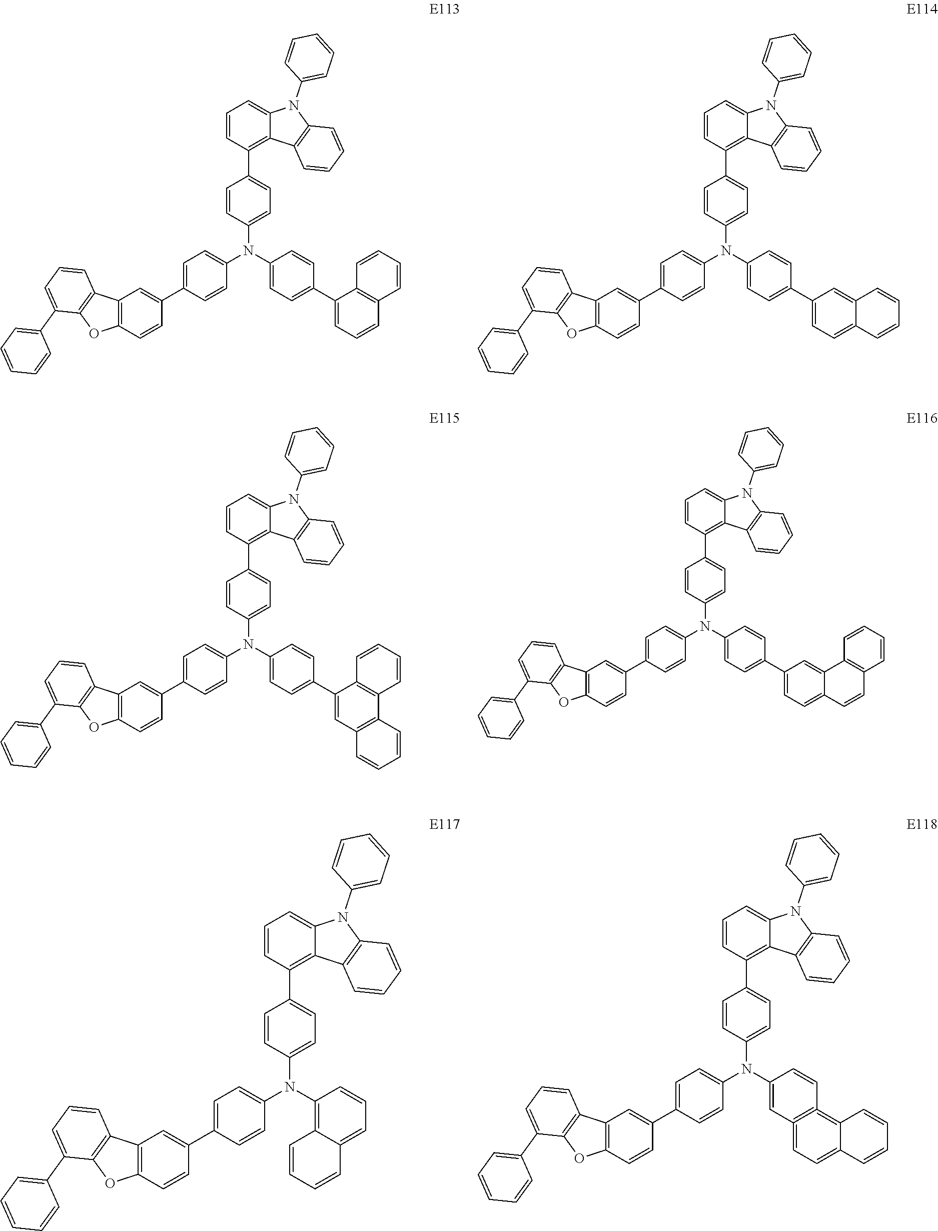

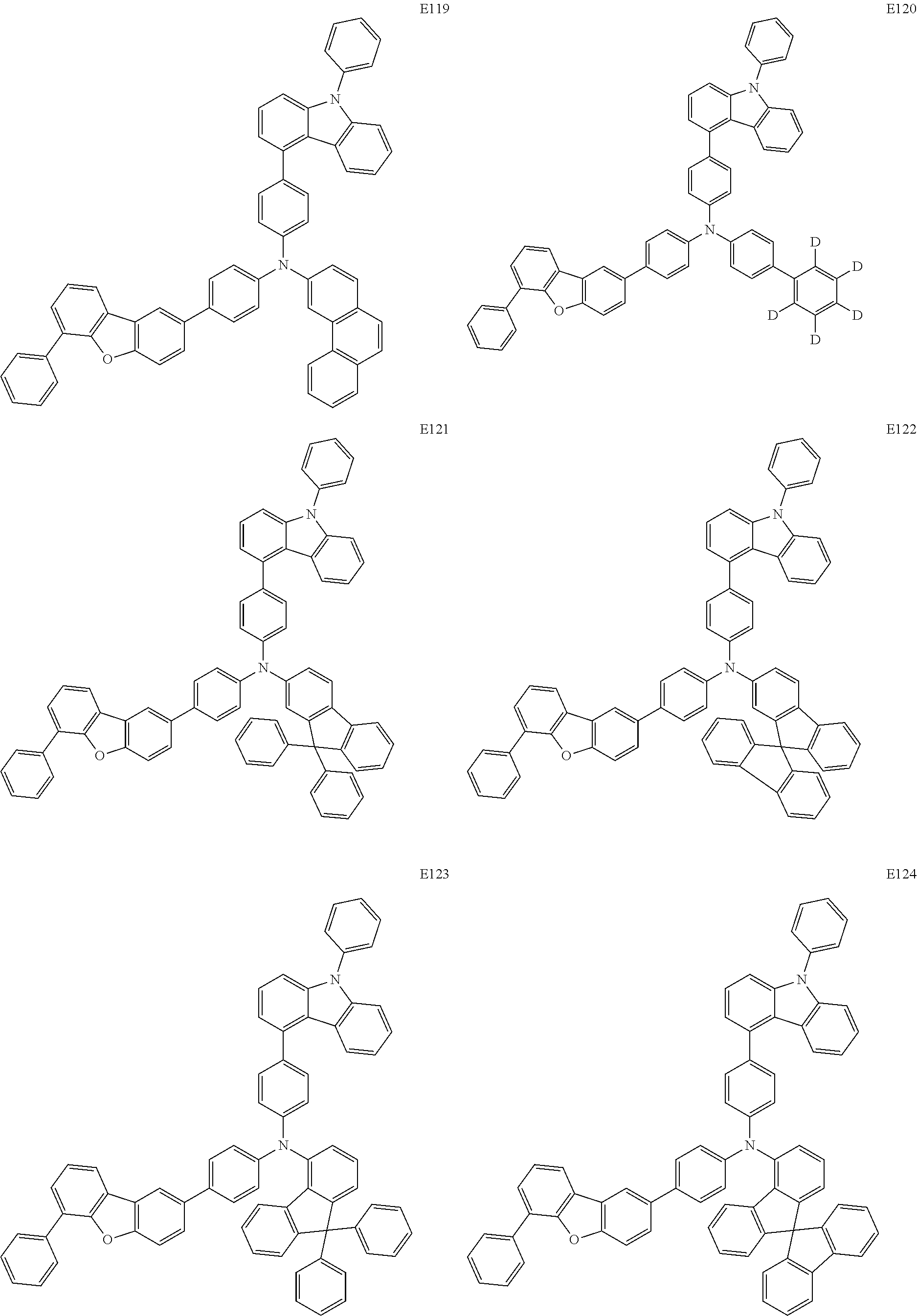

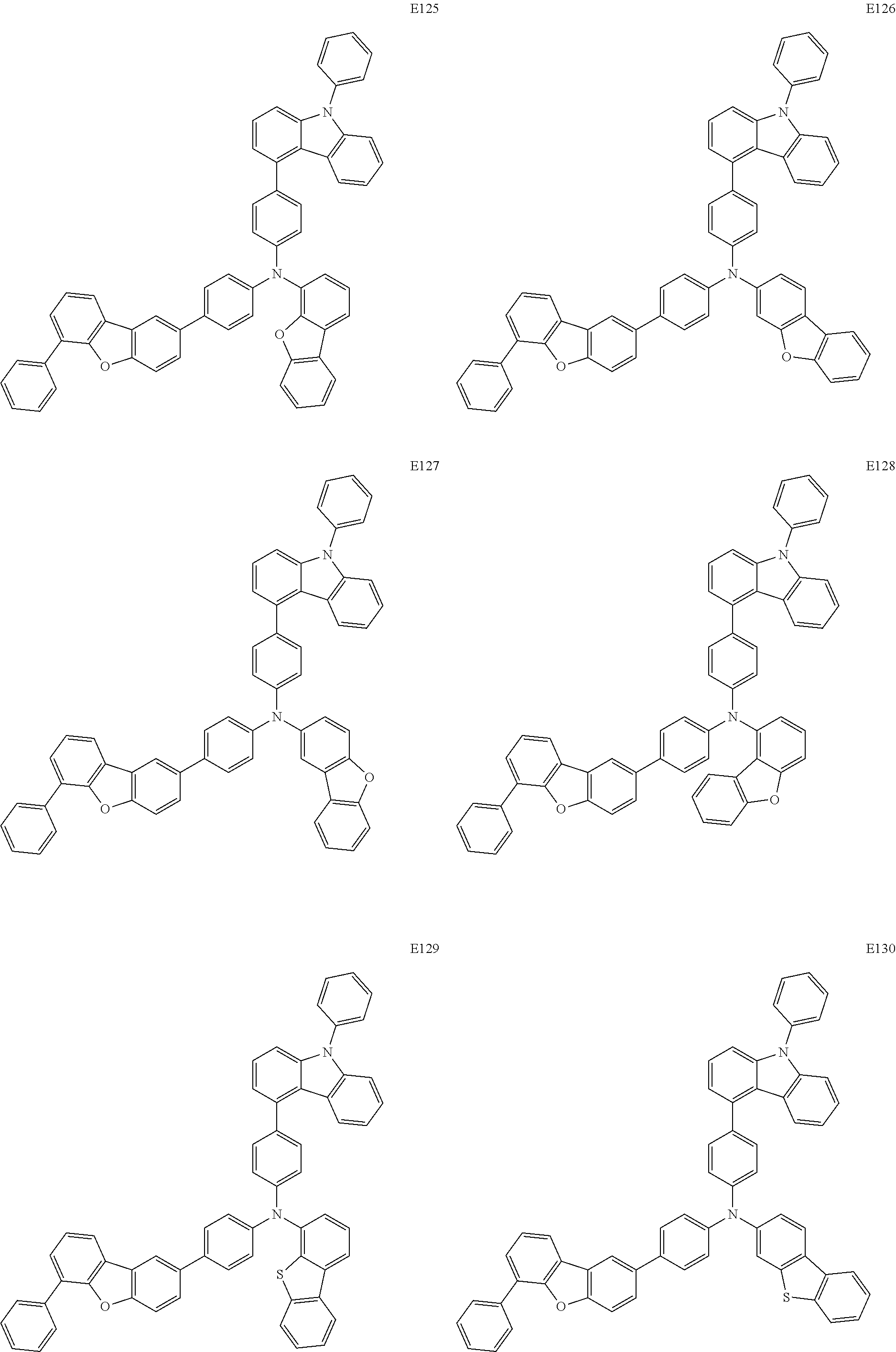

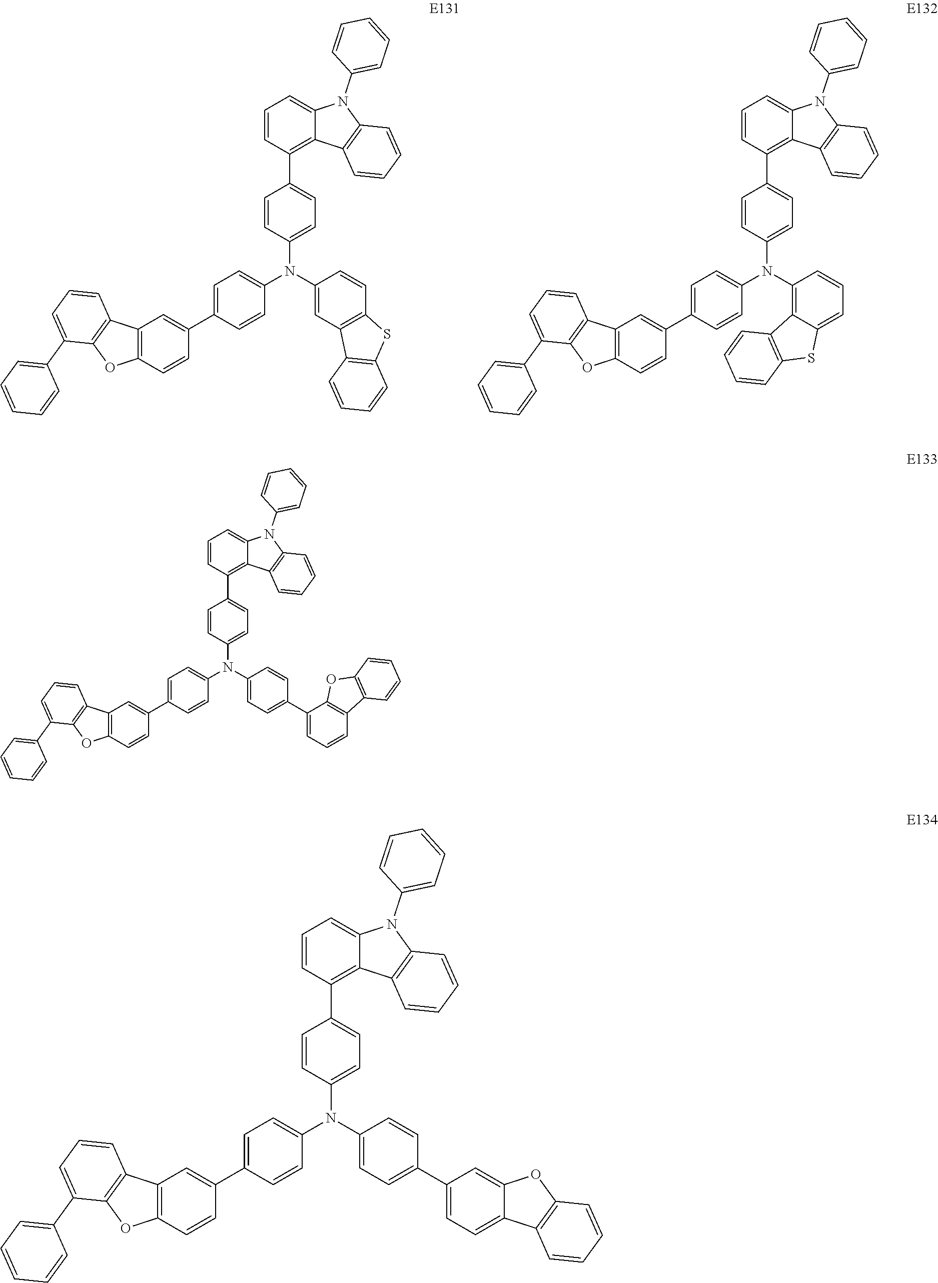 [SMILES: c1ccc(-c2cccc3c2oc2ccc(-c4ccc(N(c5ccc(-c6cccc7c6c6ccccc6n7-c6ccccc6)cc5)c5ccc6sc7ccccc7c6c5)cc4)cc23)cc1 ; c1ccc(-c2cccc3c2oc2ccc(-c4ccc(N(c5ccc(-c6cccc7c6c6ccccc6n7-c6ccccc6)cc5)c5cccc6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6ccc7c(c6)oc6ccccc67)cc5)c5ccc(-c6cccc7c6c6ccccc6n7-c6ccccc6)cc5)cc4)cc23)cc1]

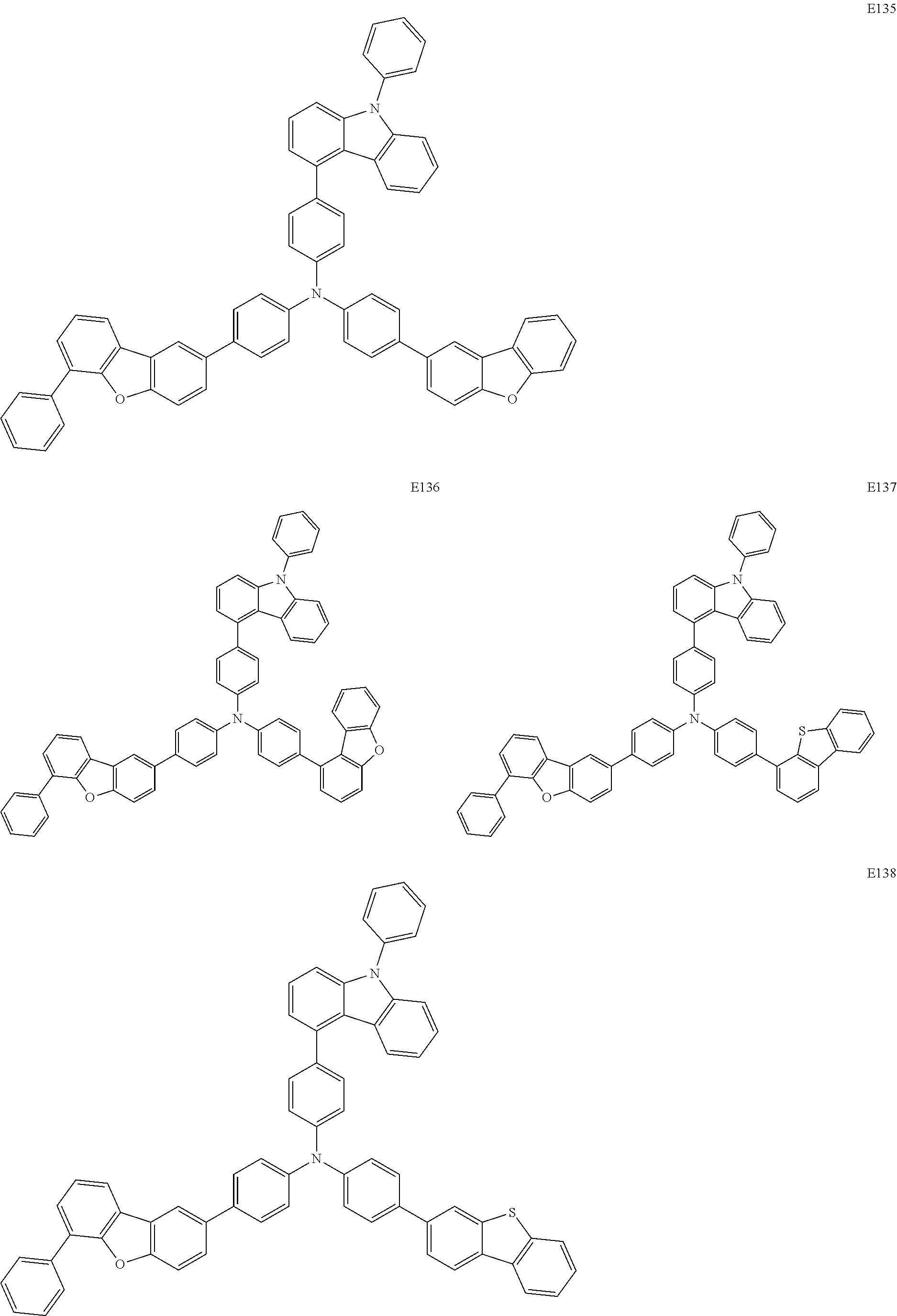 [SMILES: c1ccc(-c2cccc3c2oc2ccc(-c4ccc(N(c5ccc(-c6ccc7oc8ccccc8c7c6)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6cccc7oc8ccccc8c67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6cccc7c6sc6ccccc67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6cccc7c6c6ccccc6n7-c6ccccc6)cc5)cc4)cc23)cc1]

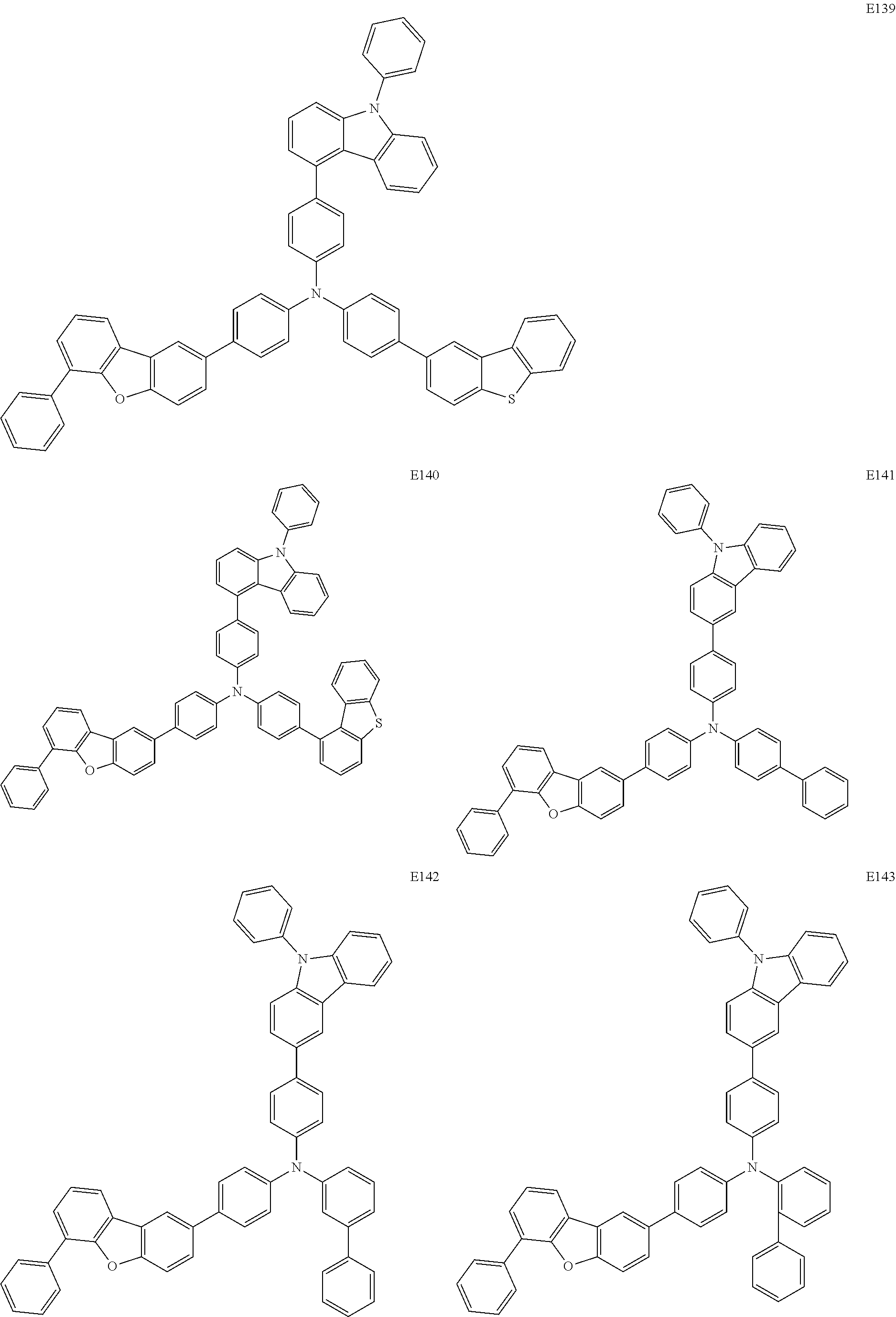

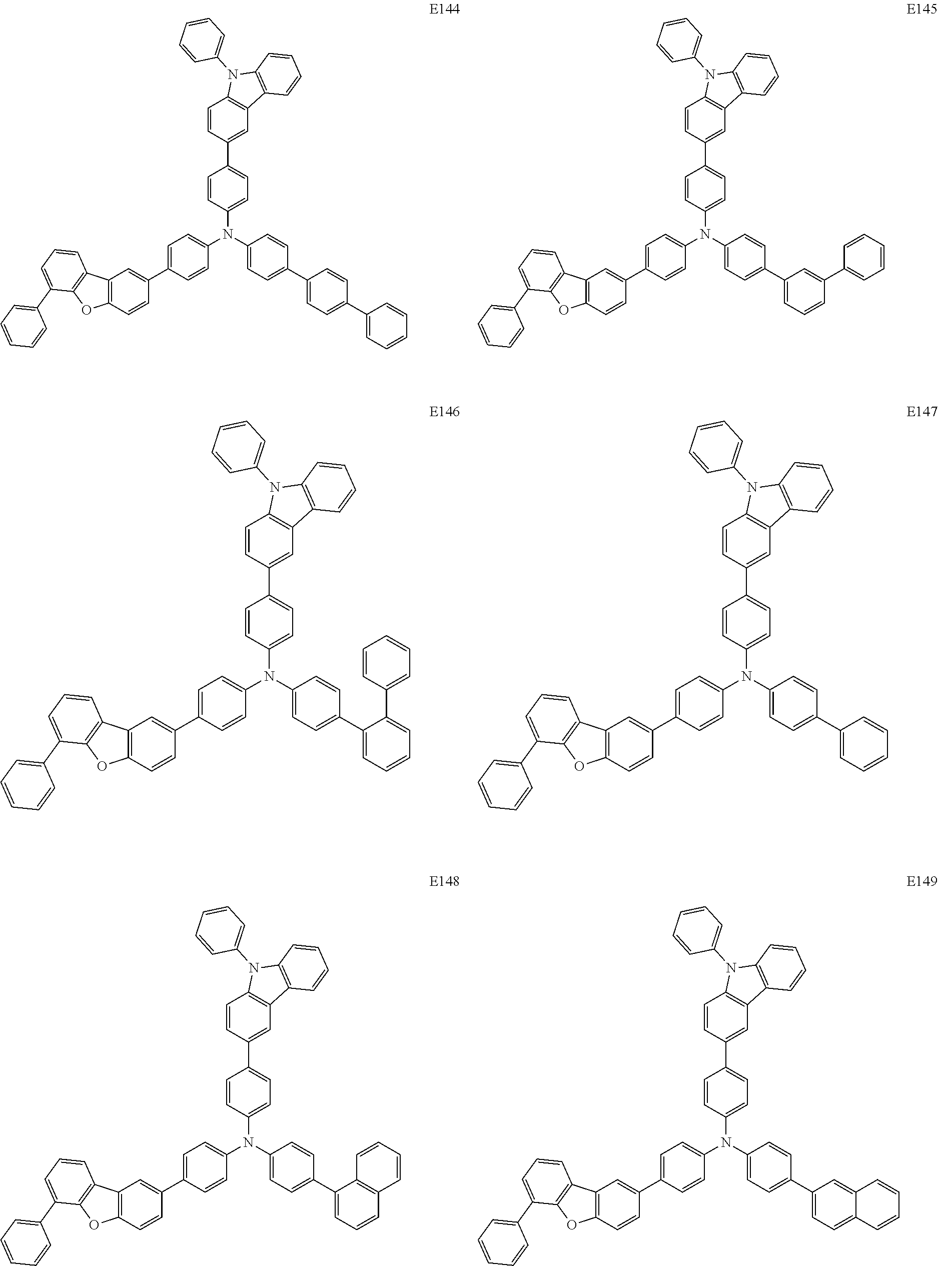

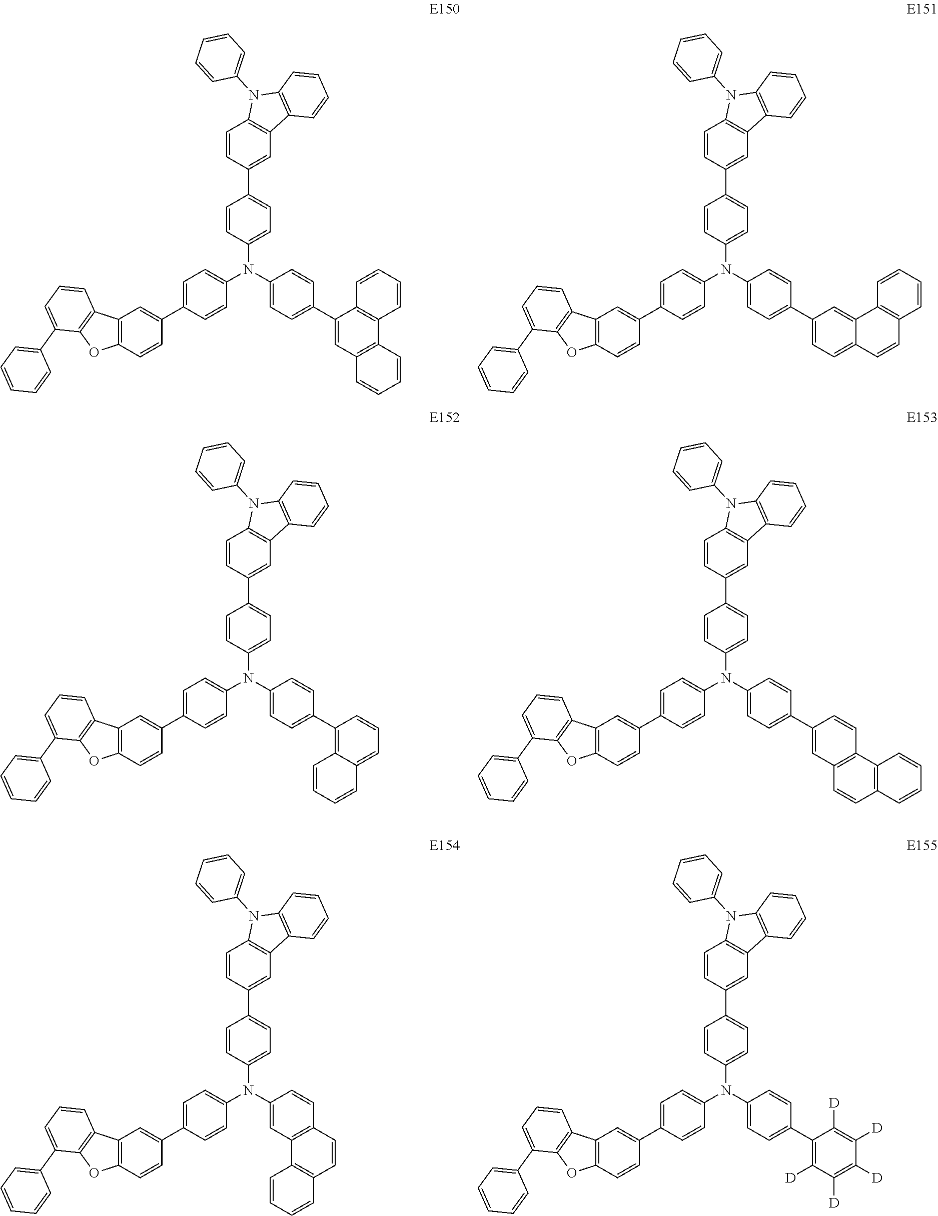

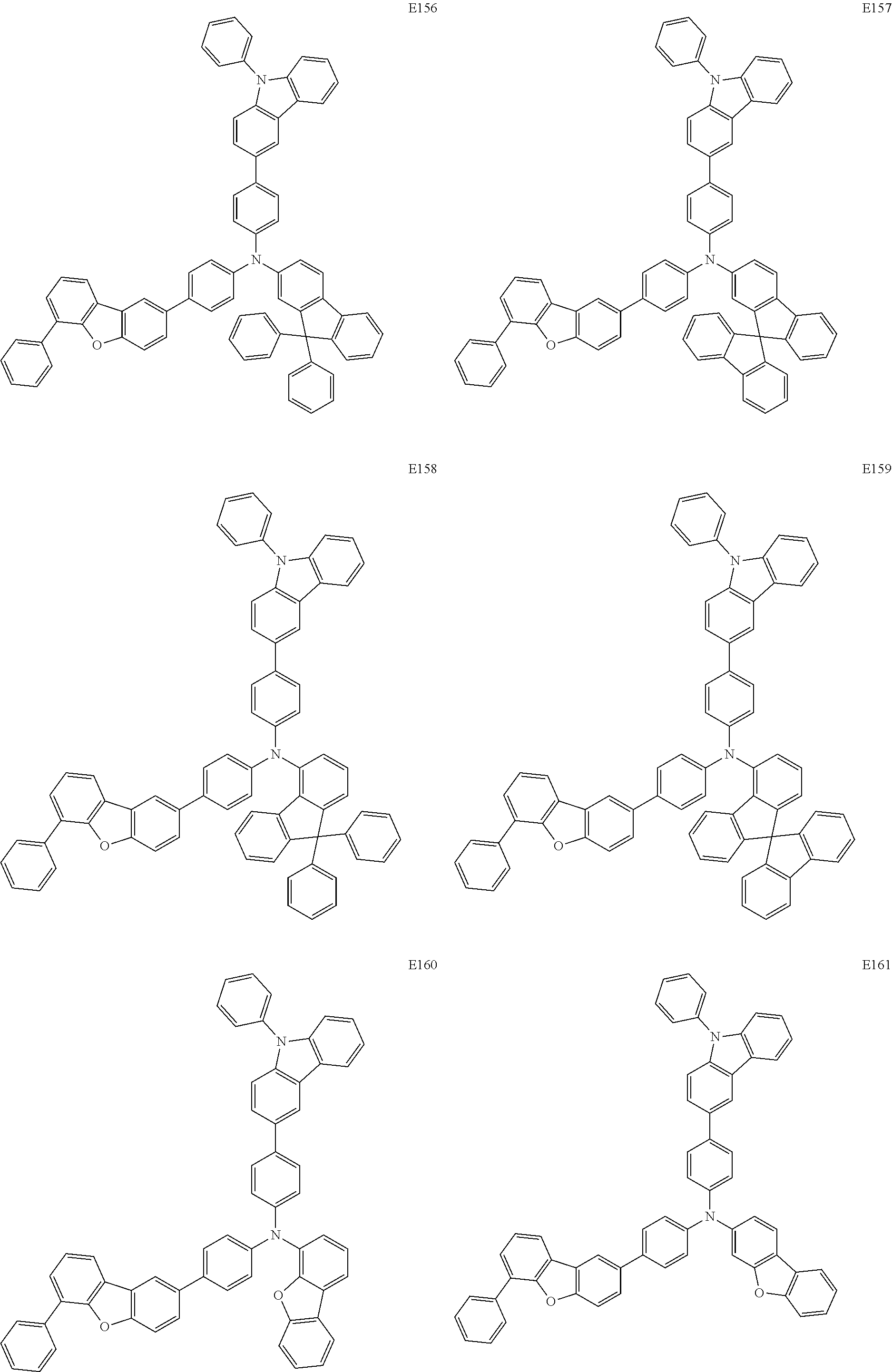

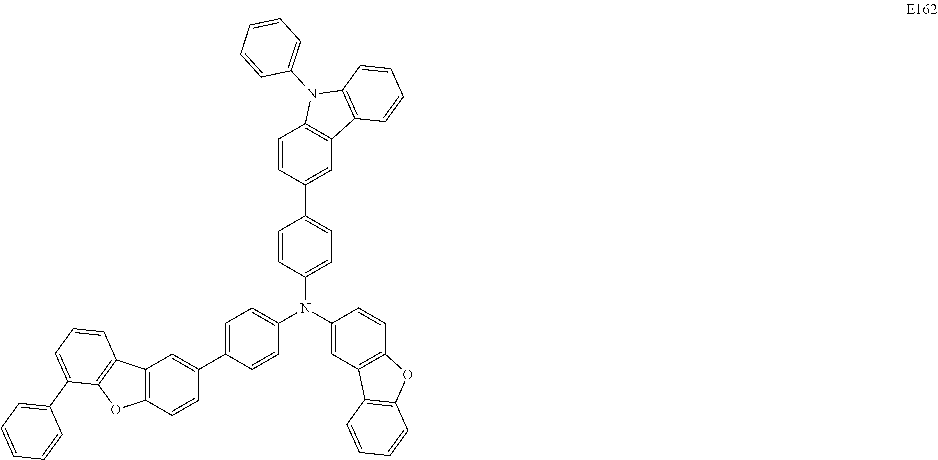 [SMILES: c1ccc(-c2cccc3c2oc2ccc(-c4ccc(N(c5ccc(-c6ccc7c(c6)c6ccccc6n7-c6ccccc6)cc5)c5ccc6oc7ccccc7c6c5)cc4)cc23)cc1]

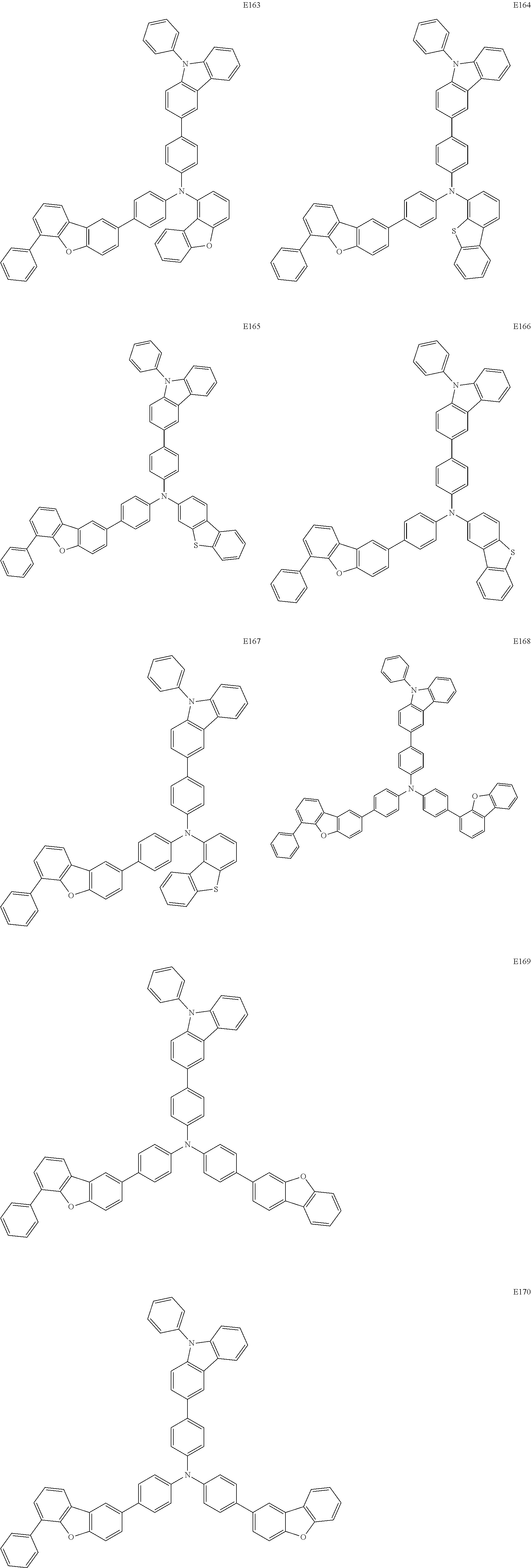

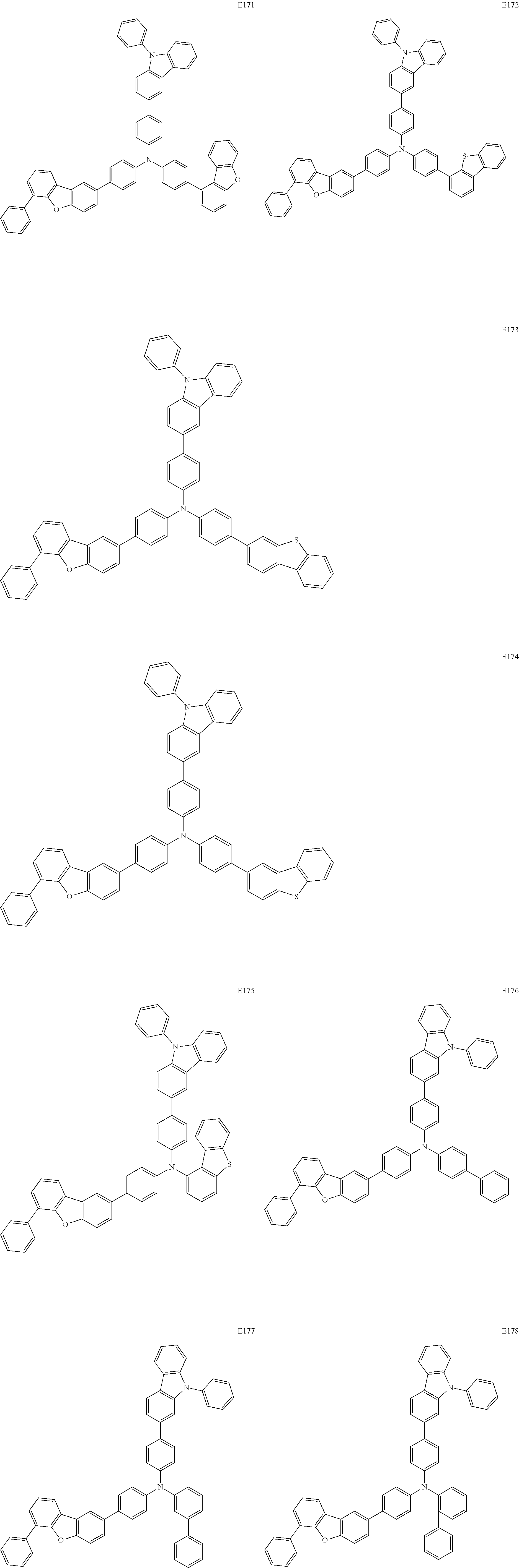

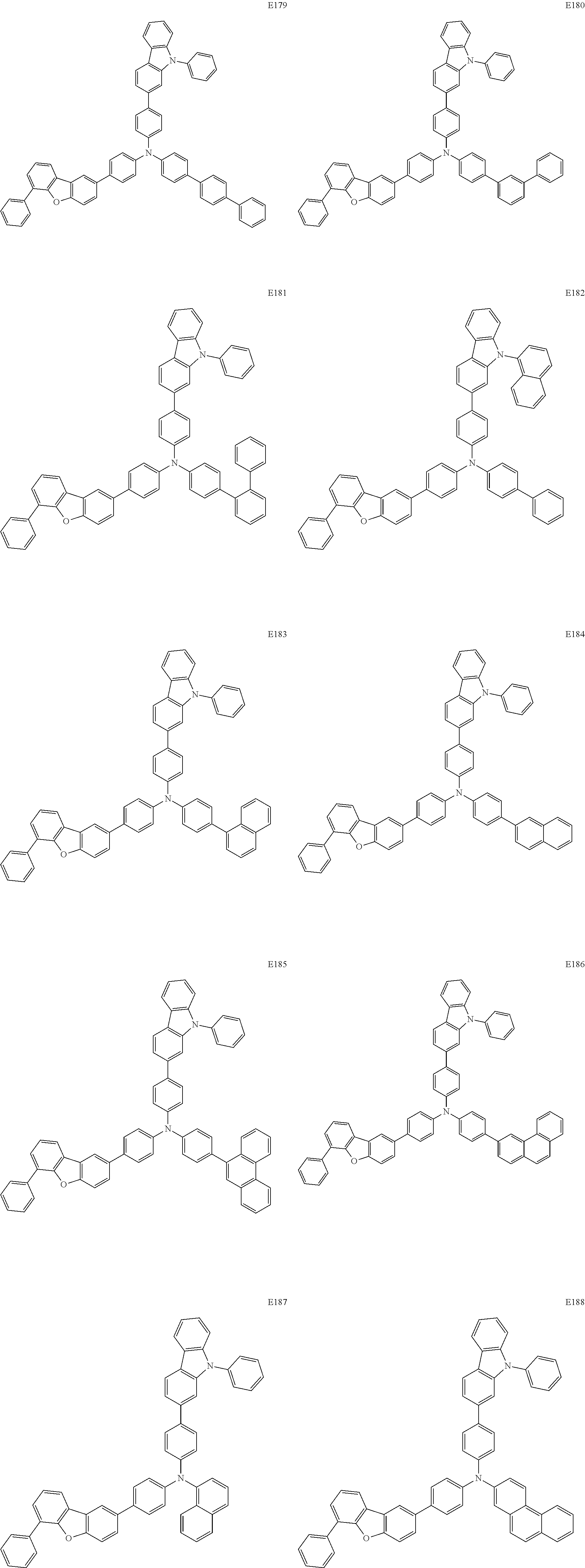

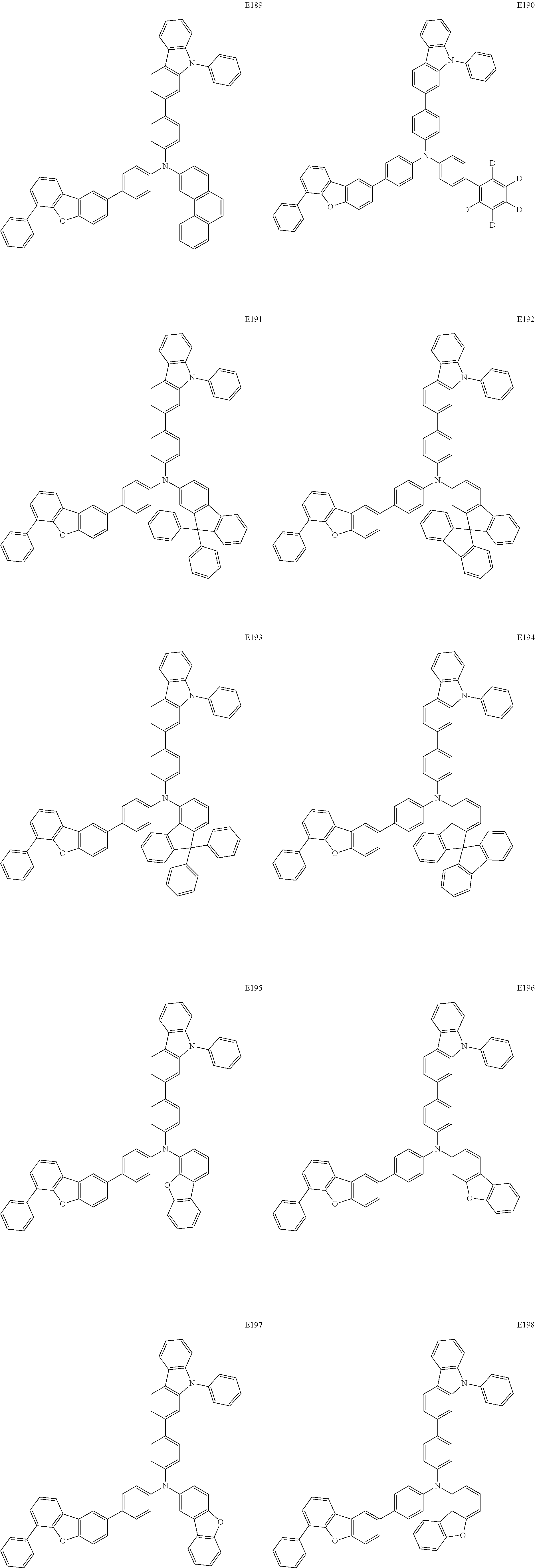

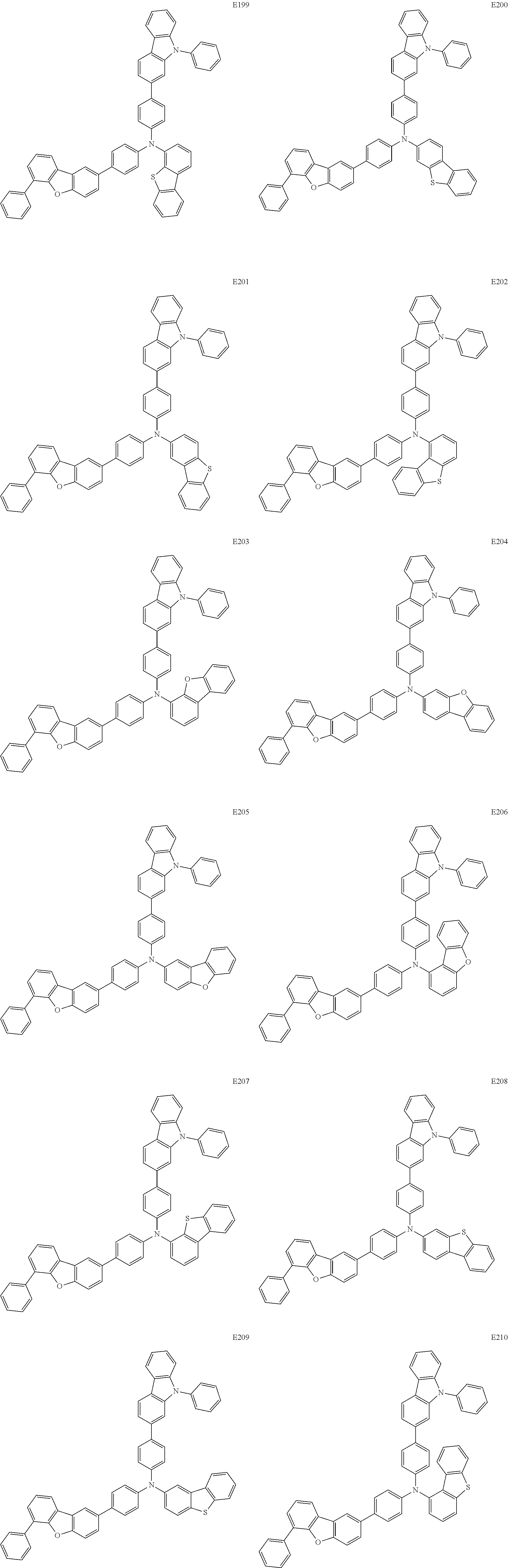

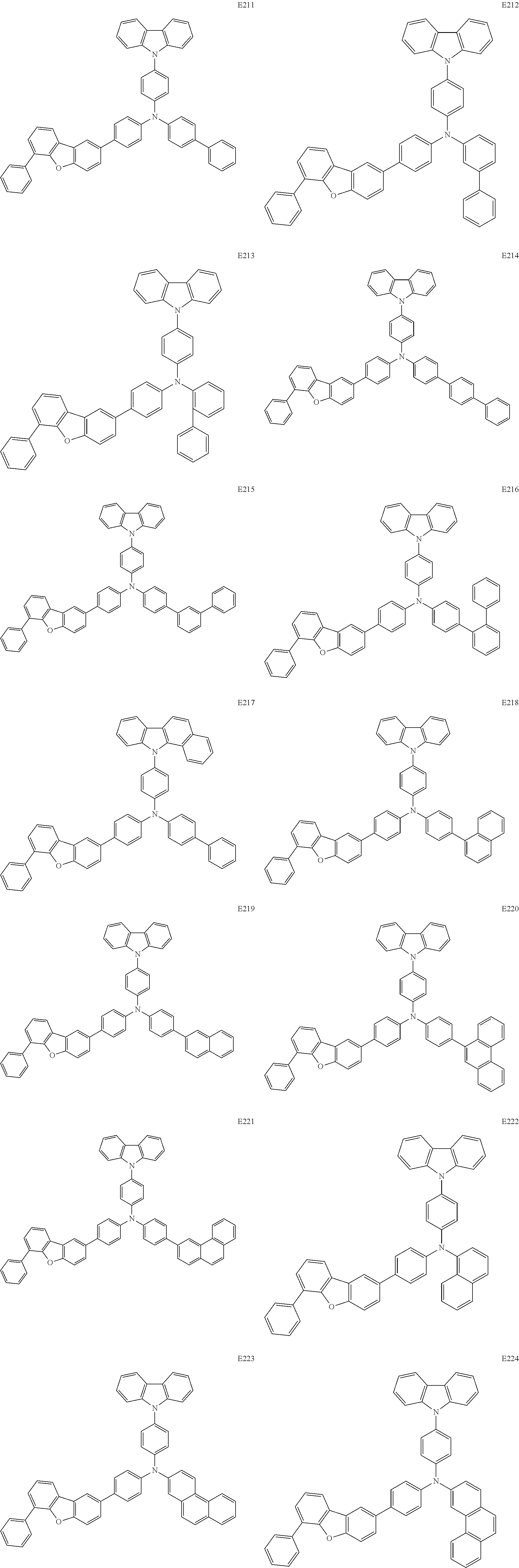

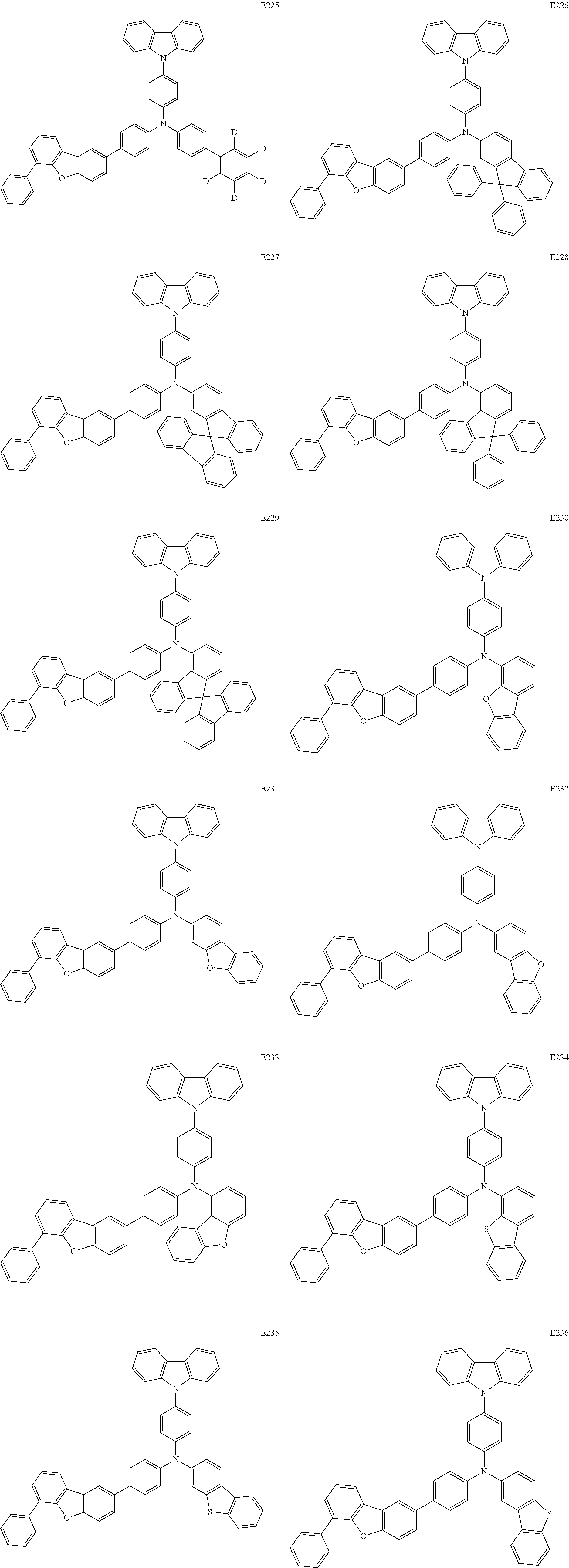

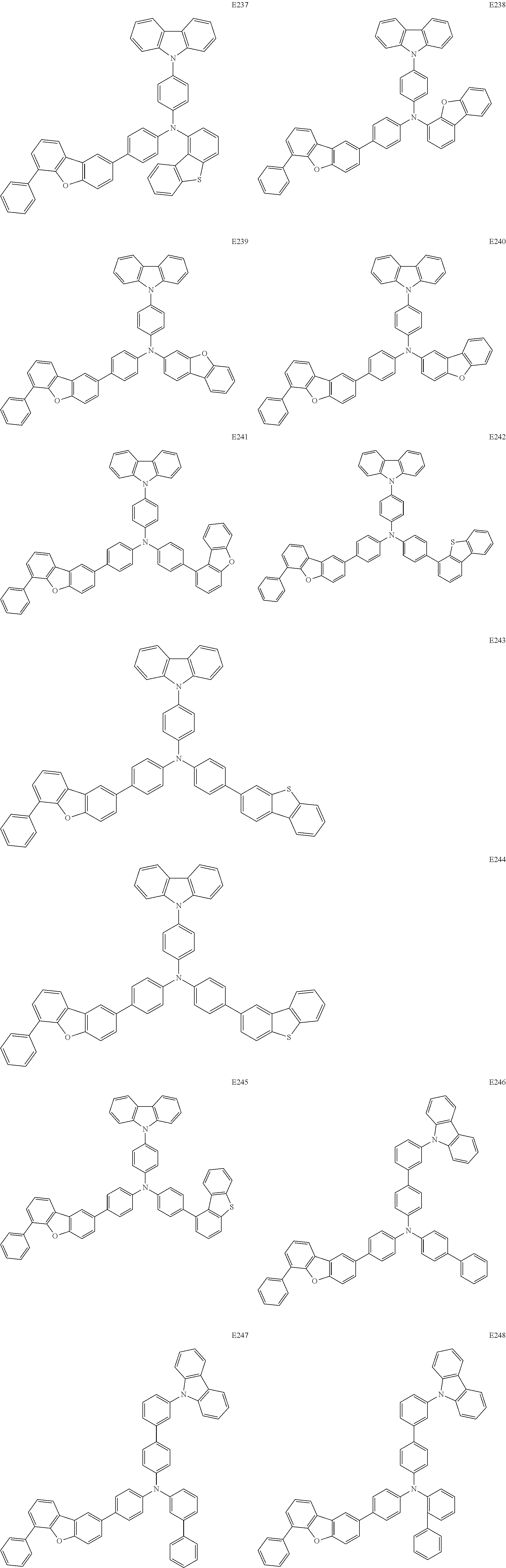

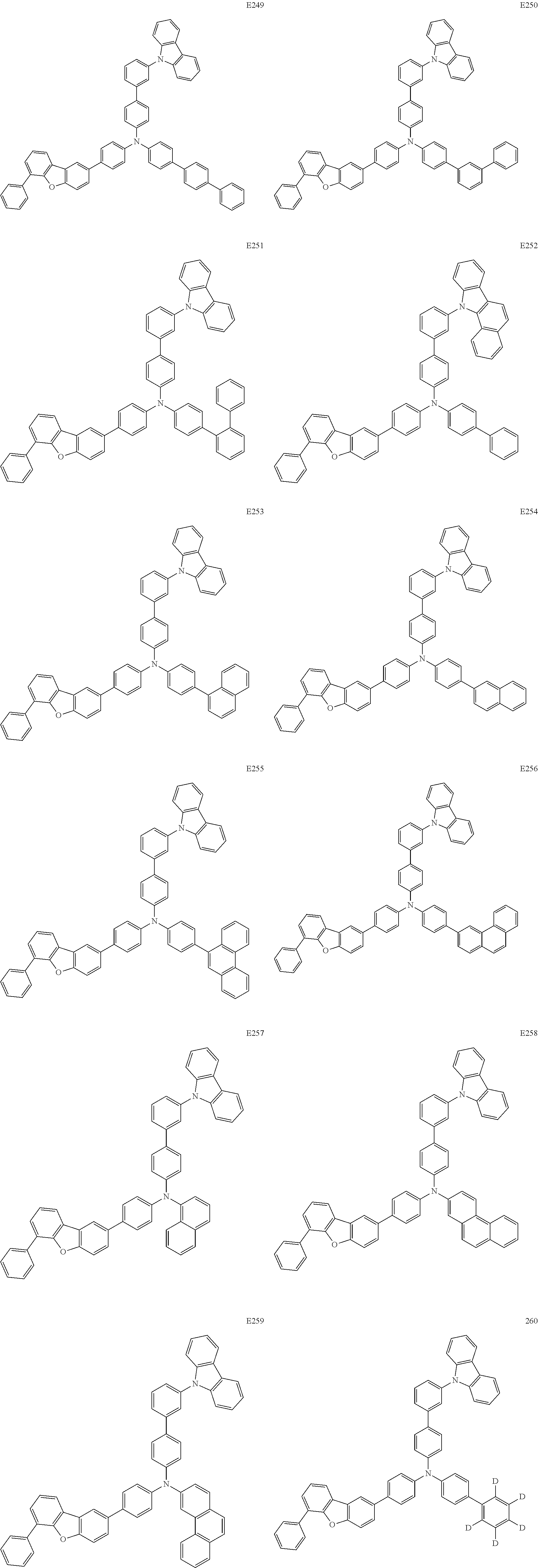

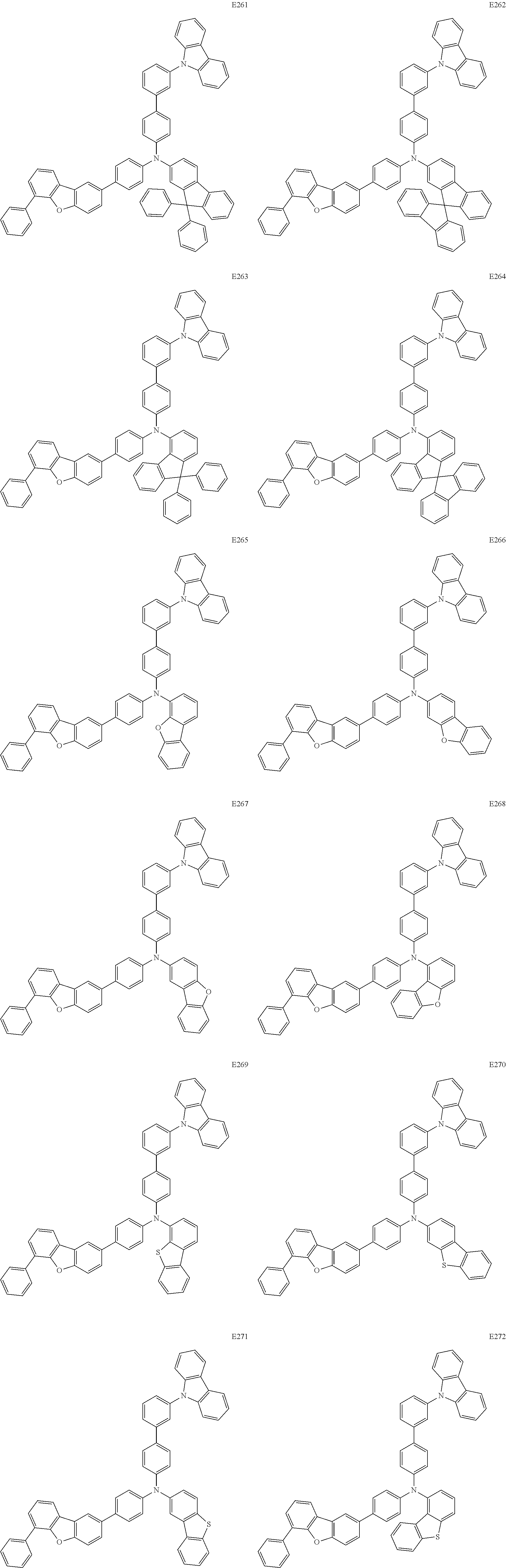

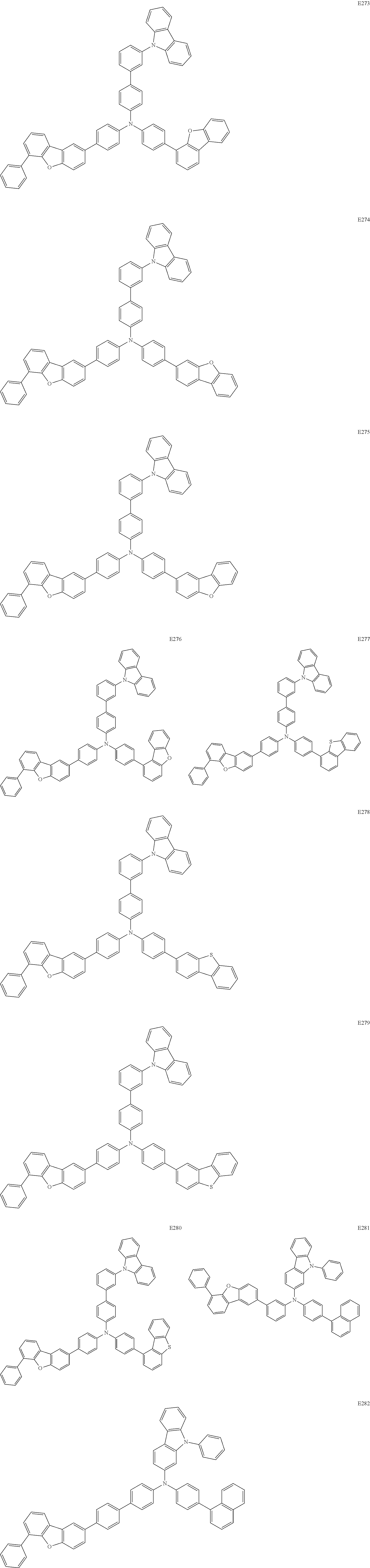

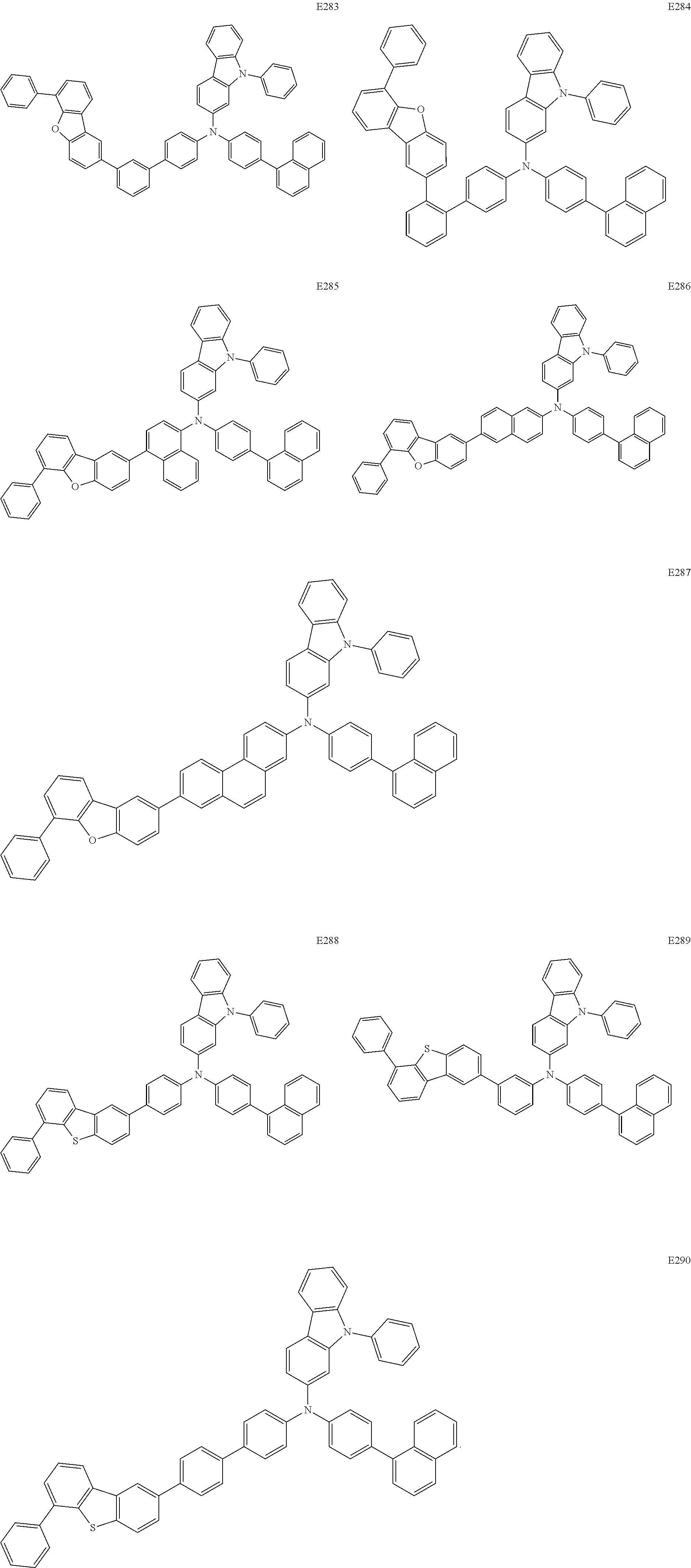

14. An amine compound represented by Formula 1:
Formula 1 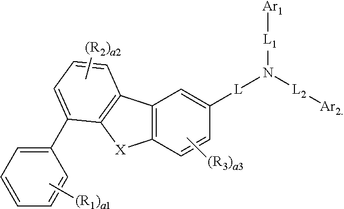
wherein, in Formula 1,
X is O or S,
R₁ to R₃ are each independently a hydrogen atom, or a deuterium atom,
a1 is an integer from 0 to 5,
a2 and a3 are each independently an integer from 0 to 3, and
L is a substituted or unsubstituted arylene group having 6 to 30 ring-forming carbon atoms,
L₁ and L₂ are each independently a direct linkage, a substituted or unsubstituted arylene group having 6 to 30 ring-forming carbon atoms, or a substituted or unsubstituted heteroarylene group having 2 to 30 ring-forming carbon atoms,
Ar₁ is represented by any one among Formula 2-1 to Formula 2-4, and
Ar₂ is a substituted or unsubstituted aryl group having 10 to 30 ring-forming carbon atoms, or a substituted or unsubstituted heteroaryl group having 2 to 30 ring-forming carbon atoms, 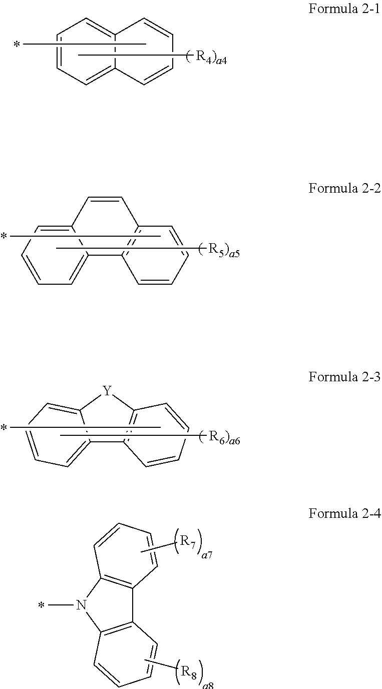
wherein, in Formula 2-1 to Formula 2-4,
R₄ to R₈ are each independently a hydrogen atom, a deuterium atom, a halogen atom, a substituted or unsubstituted alkyl group having 1 to 30 carbon group, a substituted or unsubstituted alkenyl group having 2 to 30 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring,
Y is O, S, NR₉, or CR₁₀R₁₁,
R₉ to R₁₁ are each independently a substituted or unsubstituted alkyl group having 1 to 20 carbon atoms, or a substituted or unsubstituted aryl group having 6 to 30 ring-forming carbon atoms, or are bonded to an adjacent group to form a ring,
a4 and a6 are each independently an integer from 0 to 7,
a5 is an integer from 0 to 9,
a7 and a8 are each independently an integer from 0 to 4, and [symbol]
is a position linked to Formula 1.

15. The amine compound of claim 14, wherein Ar₂ is represented by any one among Formula 3-1 to Formula 3-3 or any one among Formula 2-1 to Formula 2-4: 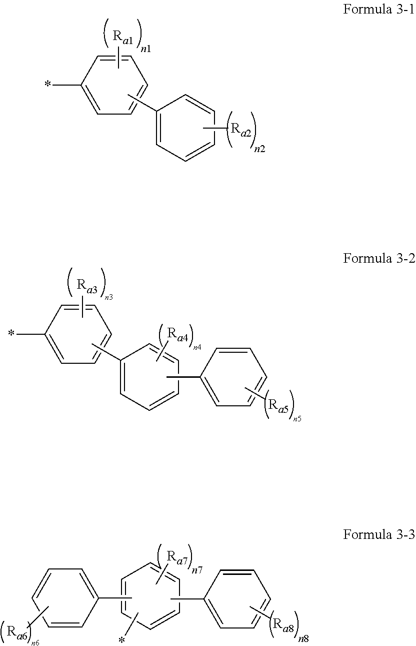
wherein, in Formula 3-1 to Formula 3-3,
Rₐ₁ to Rₐ₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1, n3 and n4 are each independently an integer from 0 to 4,
n2, n5, n6, and n8 are each independently an integer from 0 to 5,
n7 is an integer from 0 to 3, and [symbol]
is a position linked to Formula 1.

16. The amine compound of claim 14, wherein Ar₁ is represented by Formula 2-1-1 or Formula 2-1-2: 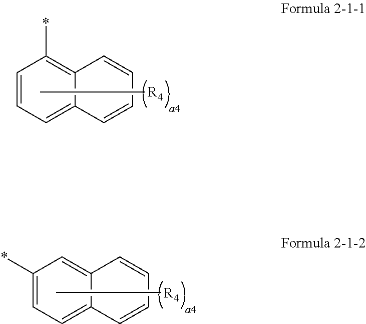
wherein, in Formula 2-1-1 and Formula 2-1-2, R₄ and a4 are the same as defined in Formula 2-1.

17. The amine compound of claim 14, wherein Ar₁ is represented by any one among Formula 2-2-1 to Formula 2-2-3: 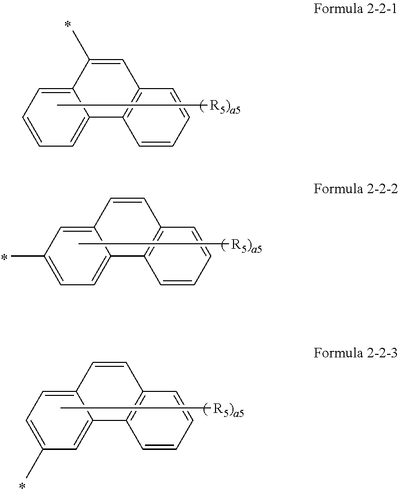
wherein, in Formula 2-2-1 to Formula 2-2-3, R₅ and a5 are the same as defined in Formula 2-2.

18. The amine compound of claim 14, wherein Ar₁ is represented by any one among Formula 2-3-1 to Formula 2-3-4: 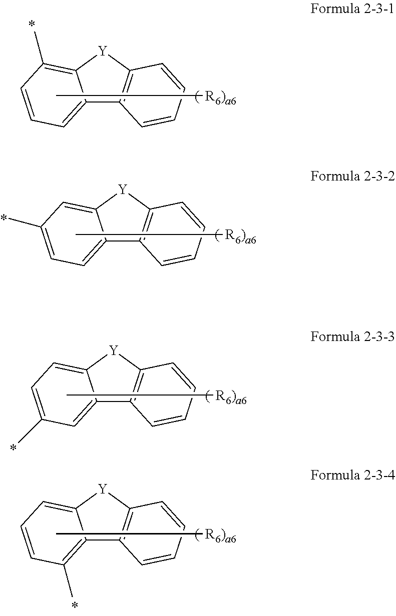
wherein, in Formula 2-3-1 to Formula 2-3-4, Y, R₆ and a6 are the same as defined in Formula 2-3.

19. The amine compound of claim 14, wherein Ar₁ is represented by any one among Formula 4-1 to Formula 4-7: 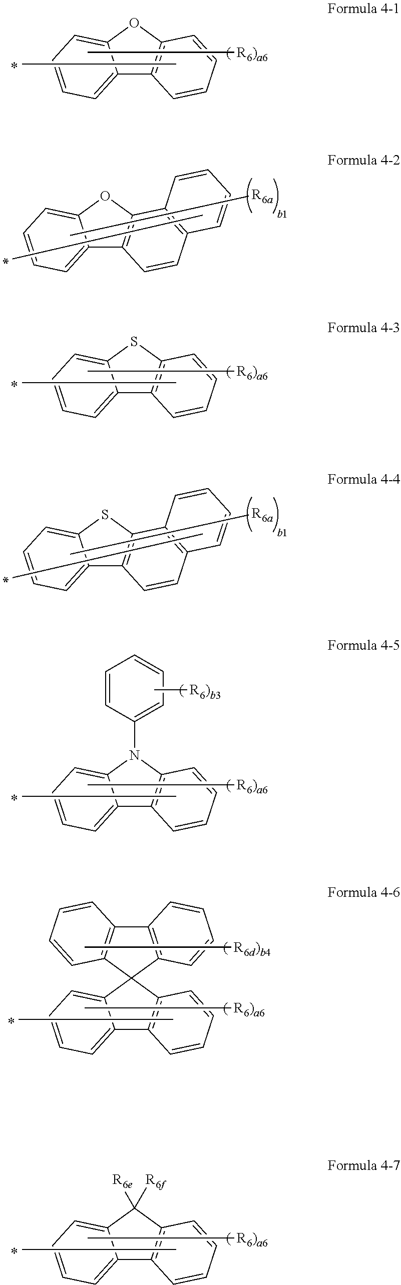
wherein, in Formula 4-1 to Formula 4-7,
R₆ₐ to R_{6d} are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
R₆ₑ and R_{6f} are each independently a substituted or unsubstituted alkyl group having 1 to 20 carbon atoms, or a substituted or unsubstituted aryl group having 6 to 30 ring-forming carbon atoms,
b1 and b2 are each independently an integer from 0 to 9,
b3 is an integer from 0 to 5,
b4 is an integer from 0 to 8, and
R₆ and a6 are the same as defined in Formula 2-3.

20. The amine compound of claim 14, wherein the amine compound represented by Formula 1 comprises at least one from among the compounds in Compound Group 1:
Compound Group 1 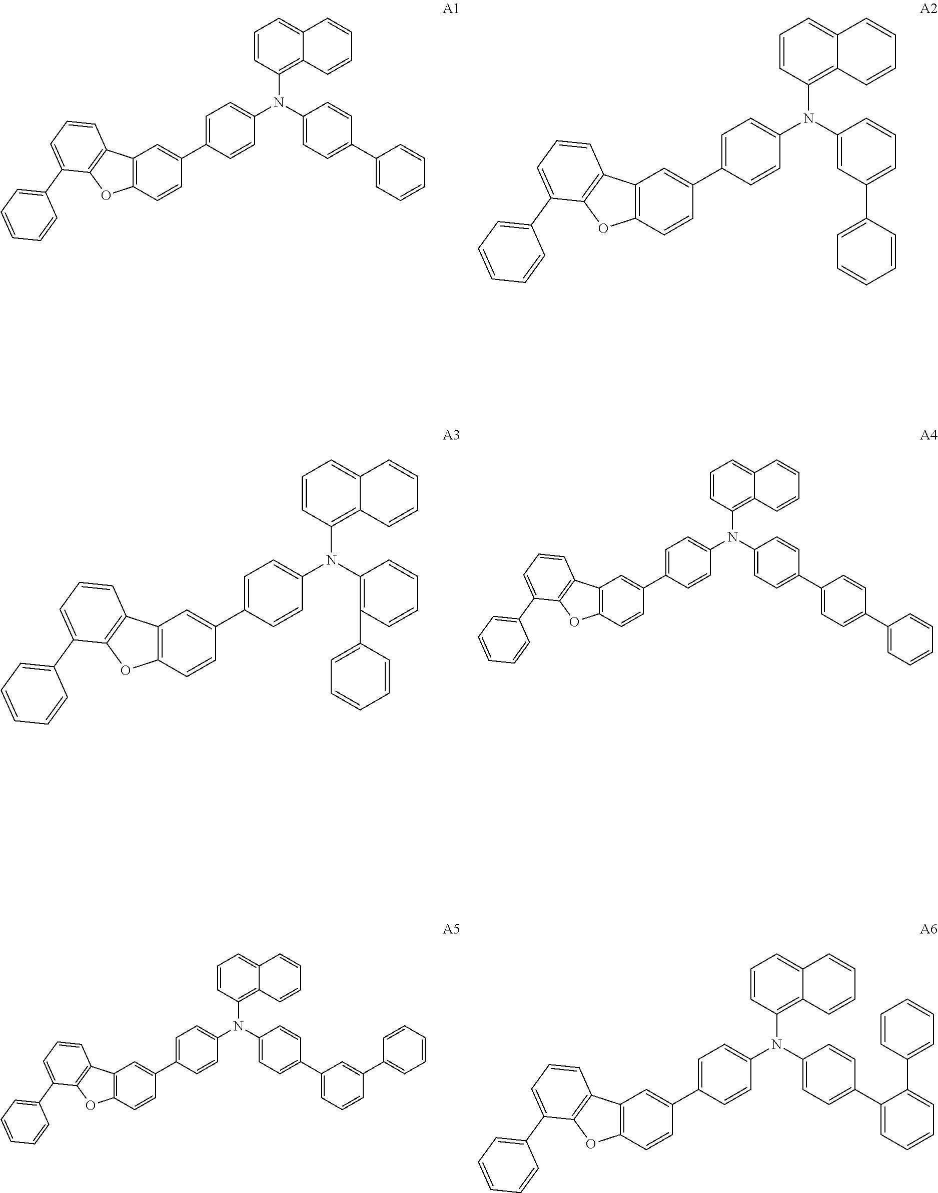 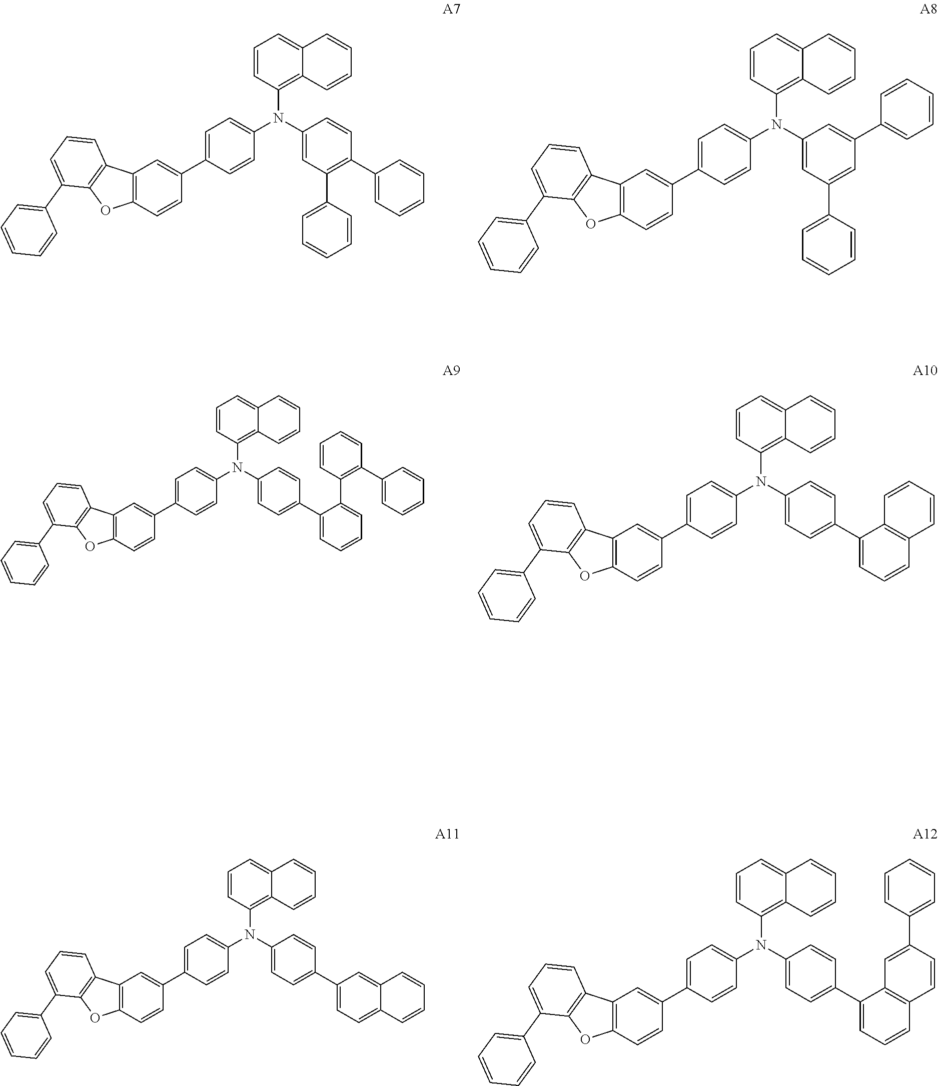 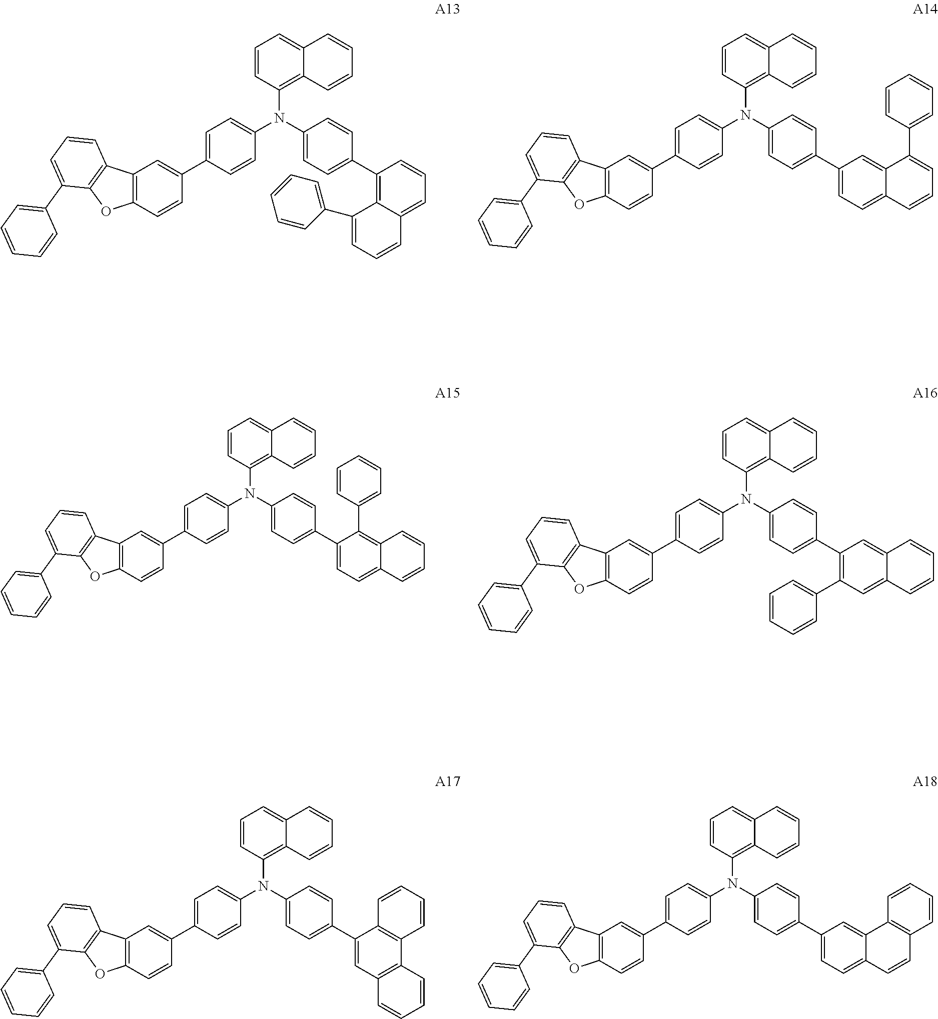 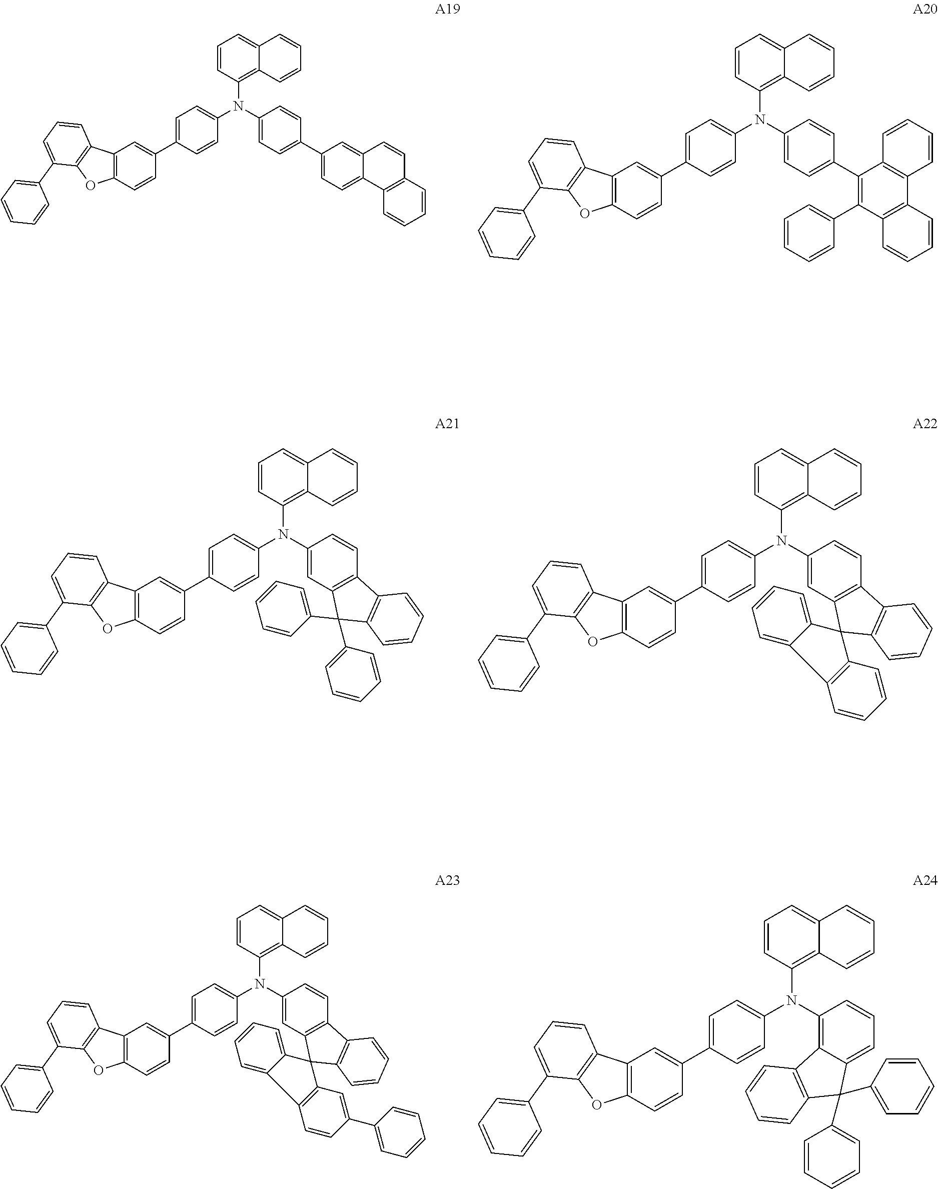 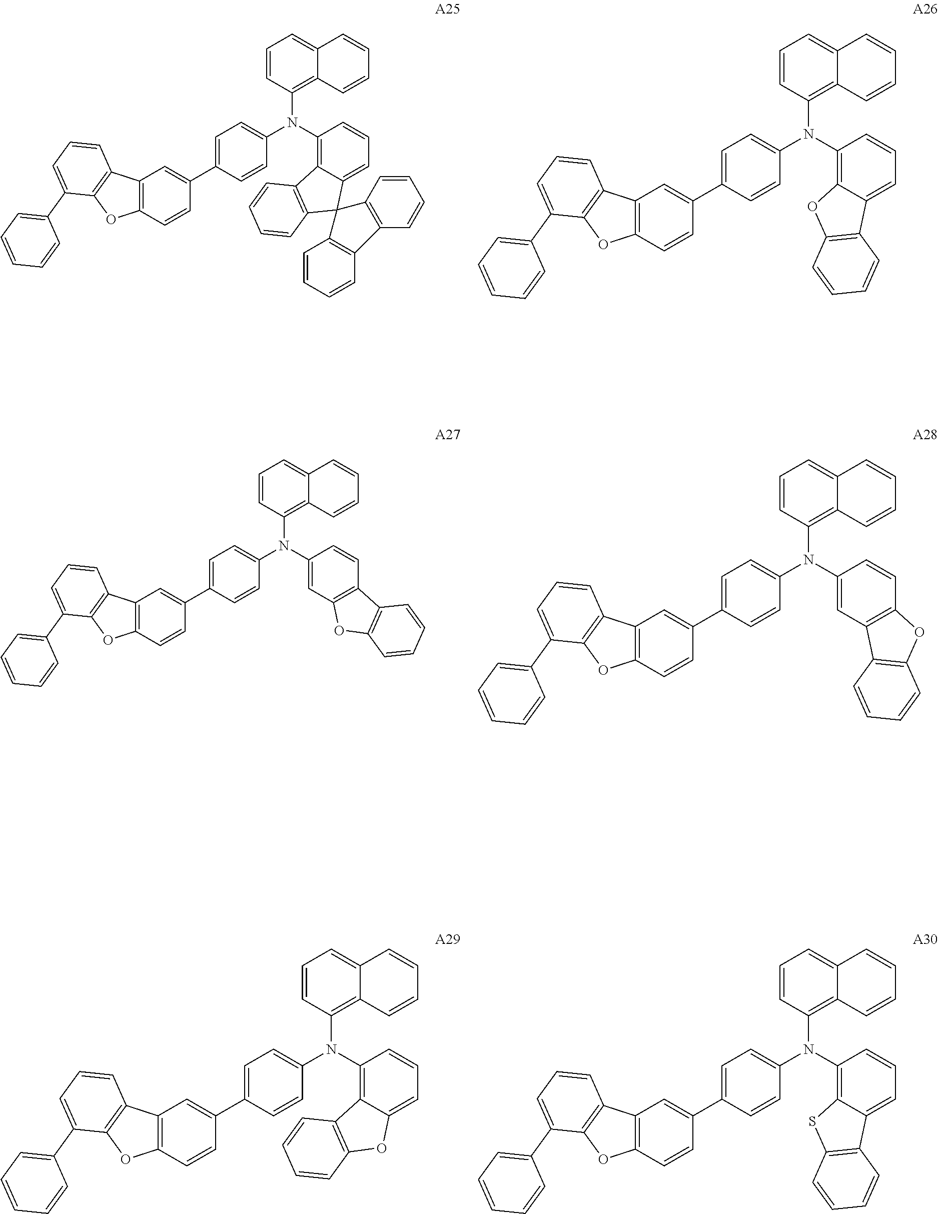 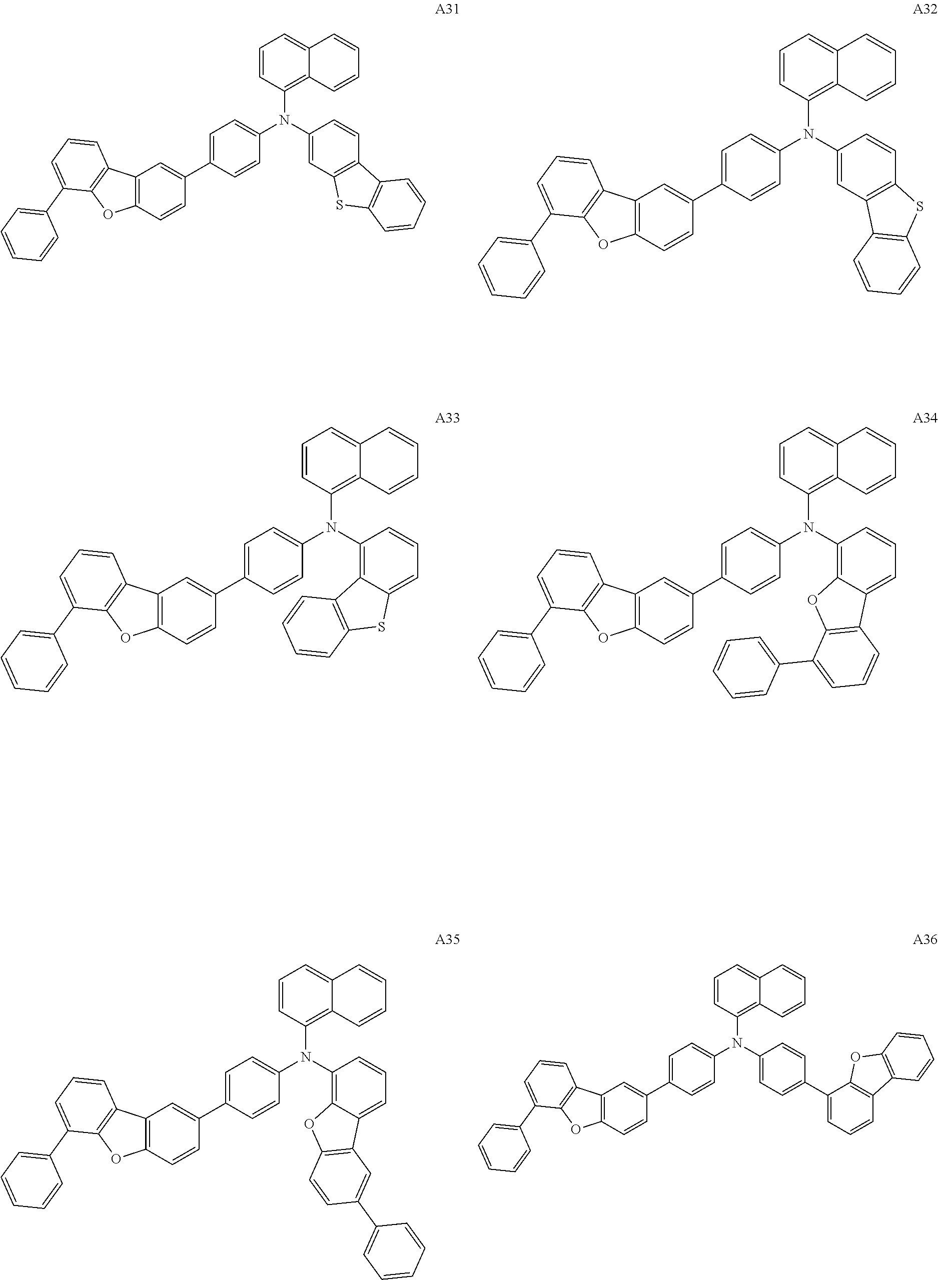 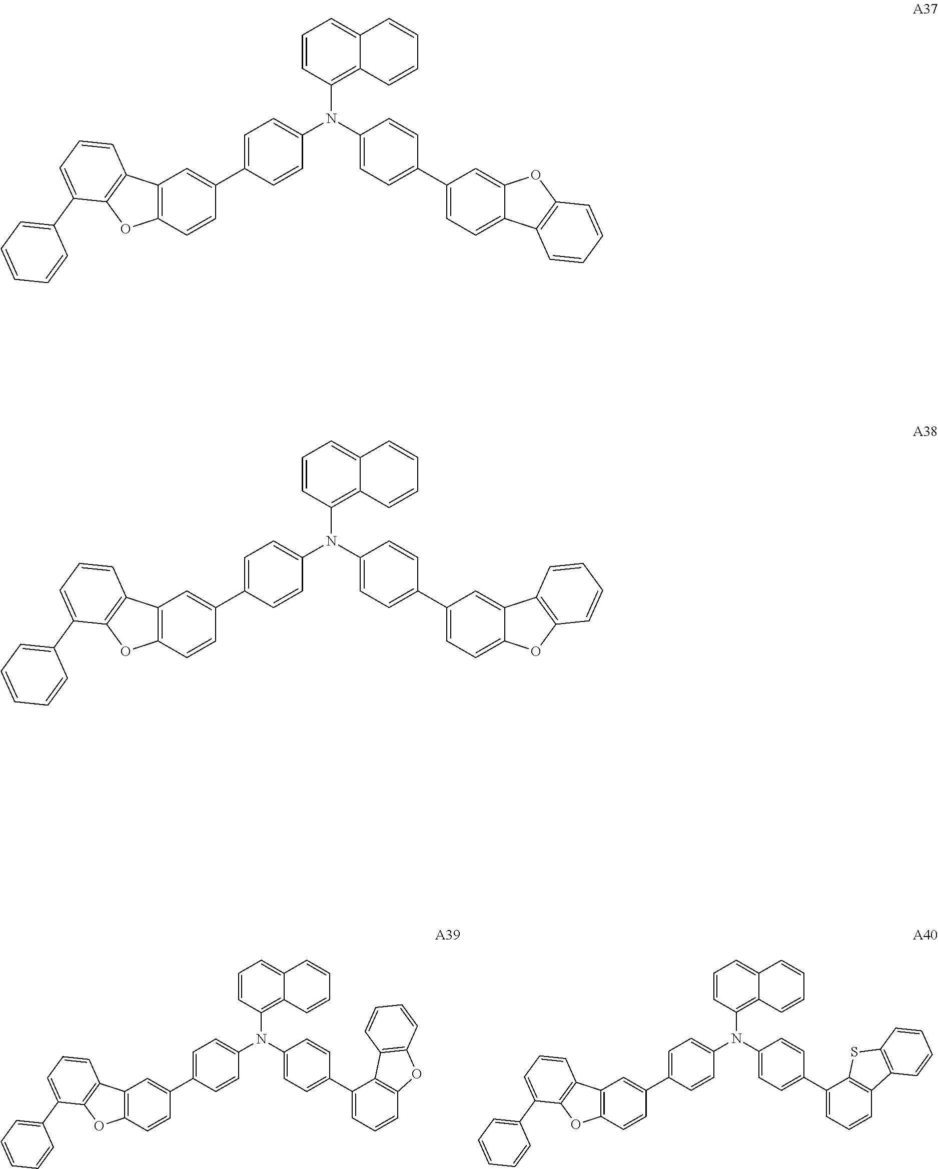 [SMILES: c1ccc(-c2cccc3c2oc2ccc(-c4ccc(N(c5ccc(-c6ccc7c(c6)oc6ccccc67)cc5)c5cccc6ccccc56)cc4)cc23)cc1 ; c1ccc(-c2cccc3c2oc2ccc(-c4ccc(N(c5ccc(-c6ccc7oc8ccccc8c7c6)cc5)c5cccc6ccccc56)cc4)cc23)cc1 ; c1ccc(-c2cccc3c2oc2ccc(-c4ccc(N(c5ccc(-c6cccc7oc8ccccc8c67)cc5)c5cccc6ccccc56)cc4)cc23)cc1 ; c1ccc(-c2cccc3c2oc2ccc(-c4ccc(N(c5ccc(-c6cccc7c6sc6ccccc67)cc5)c5cccc6ccccc56)cc4)cc23)cc1] 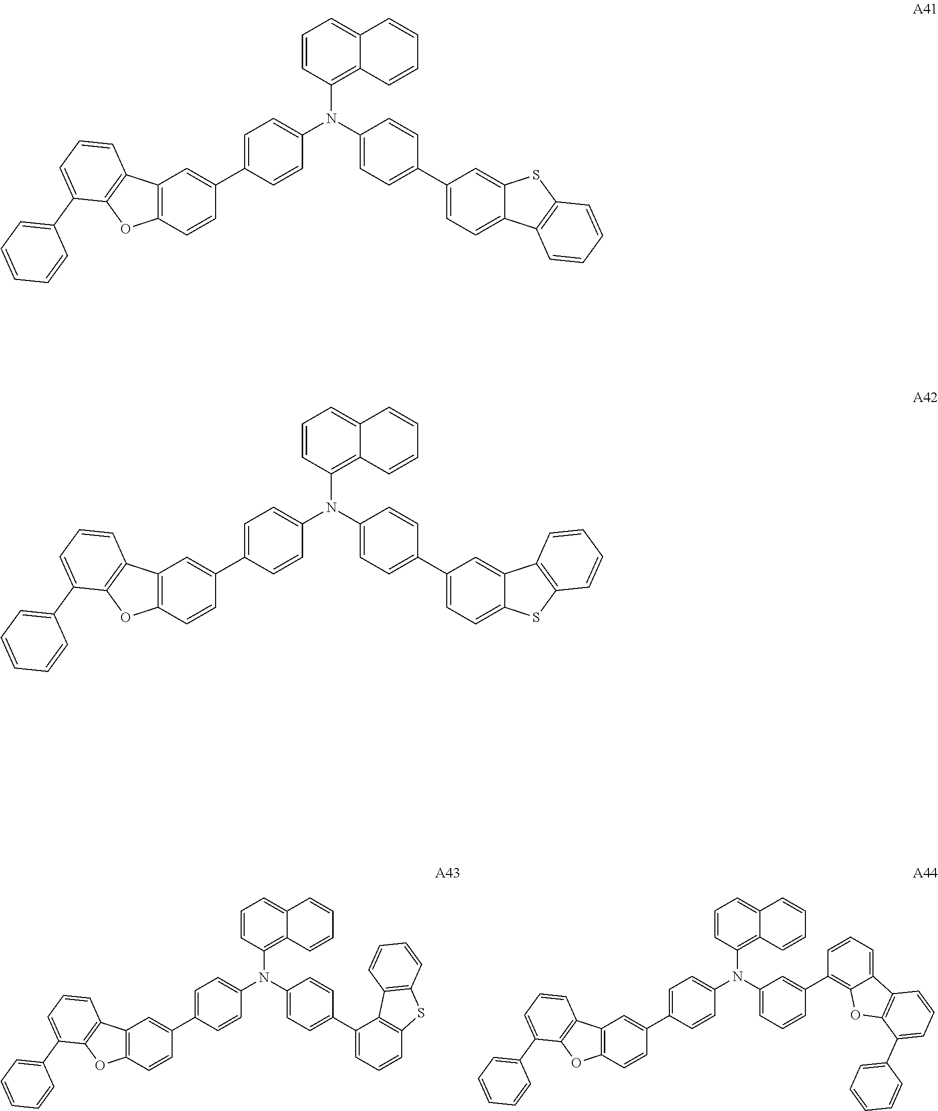 [SMILES: c1ccc(-c2cccc3c2oc2ccc(-c4ccc(N(c5ccc(-c6ccc7c(c6)sc6ccccc67)cc5)c5cccc6ccccc56)cc4)cc23)cc1 ; c1ccc(-c2cccc3c2oc2ccc(-c4ccc(N(c5ccc(-c6ccc7sc8ccccc8c7c6)cc5)c5cccc6ccccc56)cc4)cc23)cc1 ; c1ccc(-c2cccc3c2oc2ccc(-c4ccc(N(c5ccc(-c6cccc7sc8ccccc8c67)cc5)c5cccc6ccccc56)cc4)cc23)cc1 ; c1ccc(-c2cccc3c2oc2ccc(-c4ccc(N(c5cccc(-c6cccc7c6oc6c(-c8ccccc8)cccc67)c5)c5cccc6ccccc56)cc4)cc23)cc1] 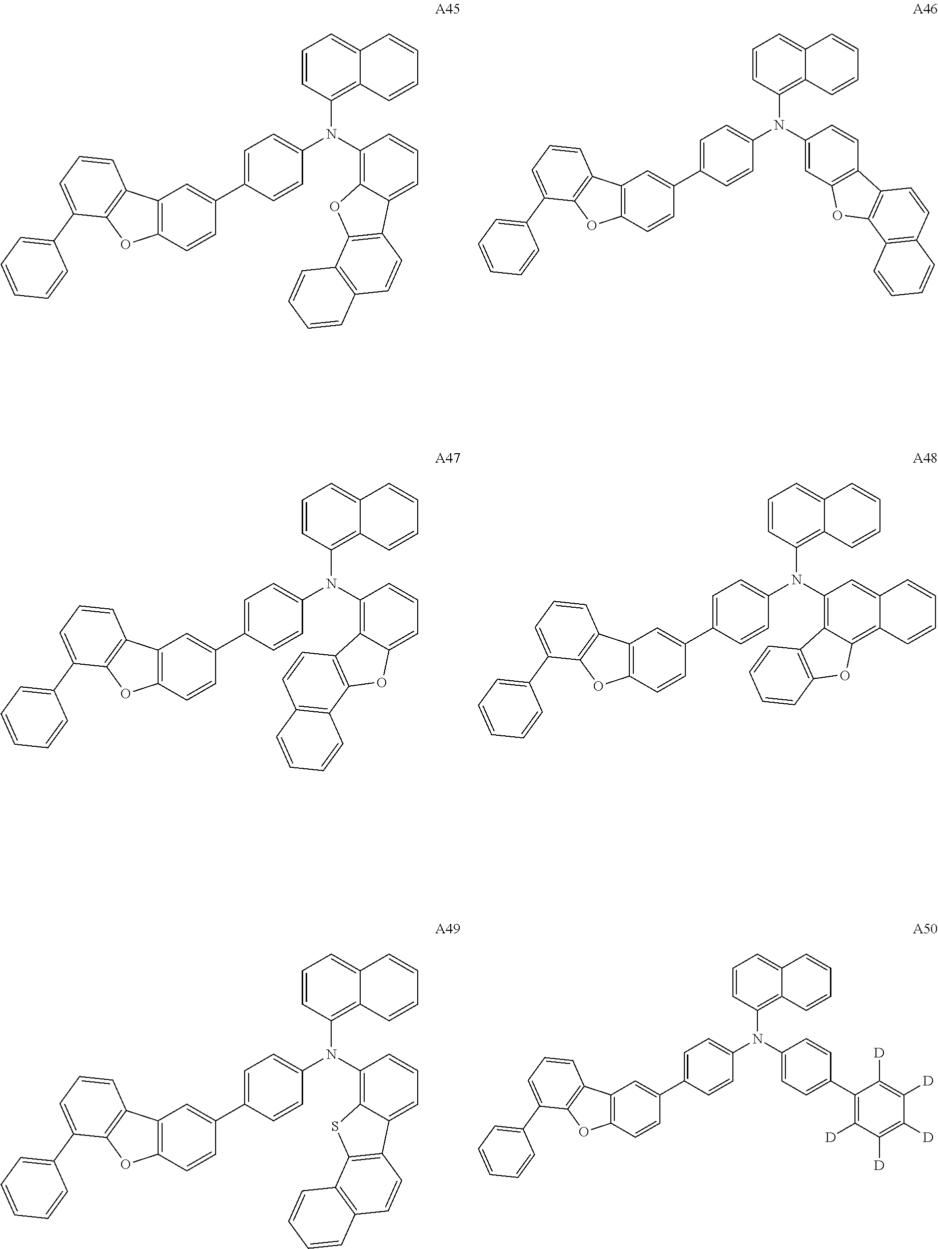 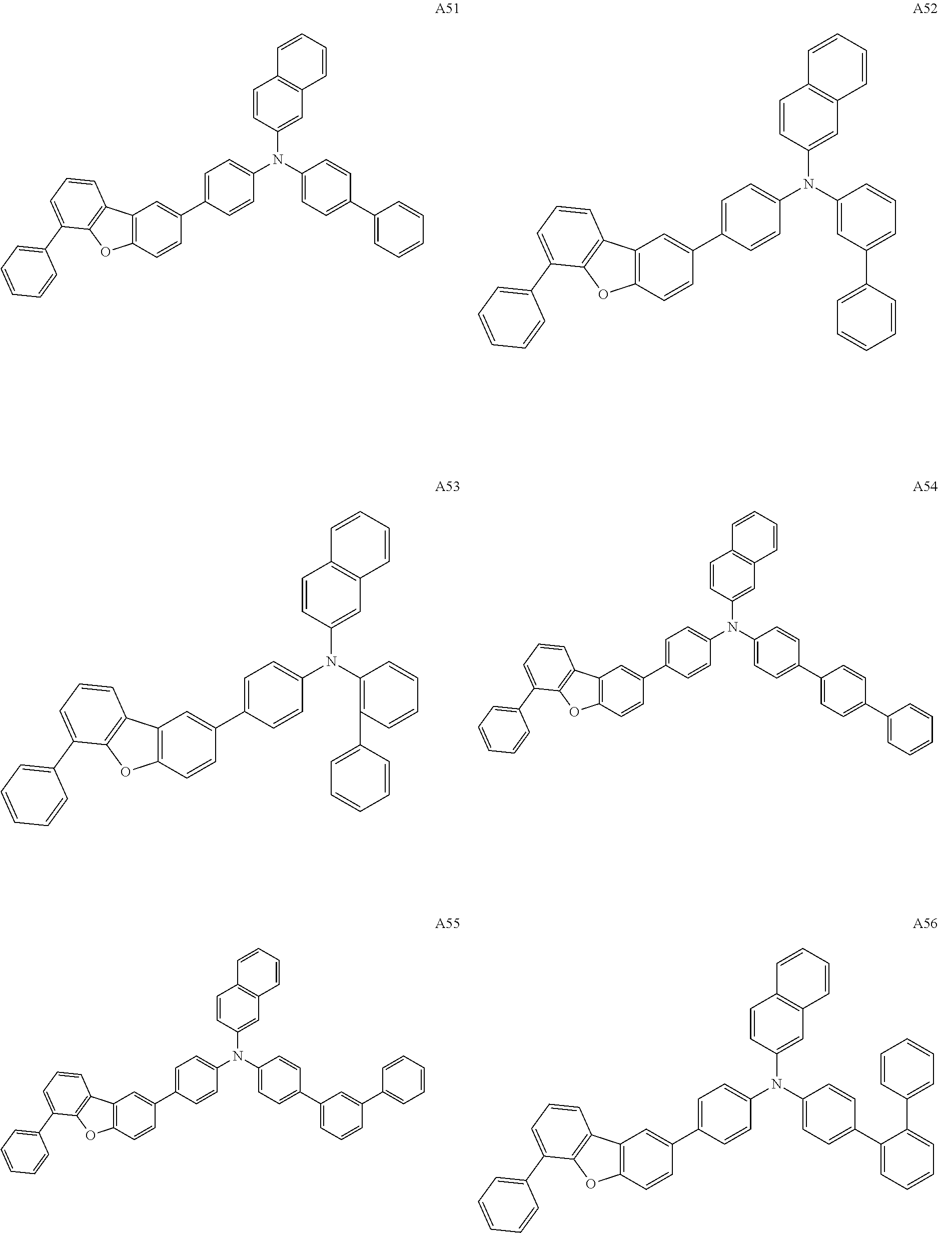 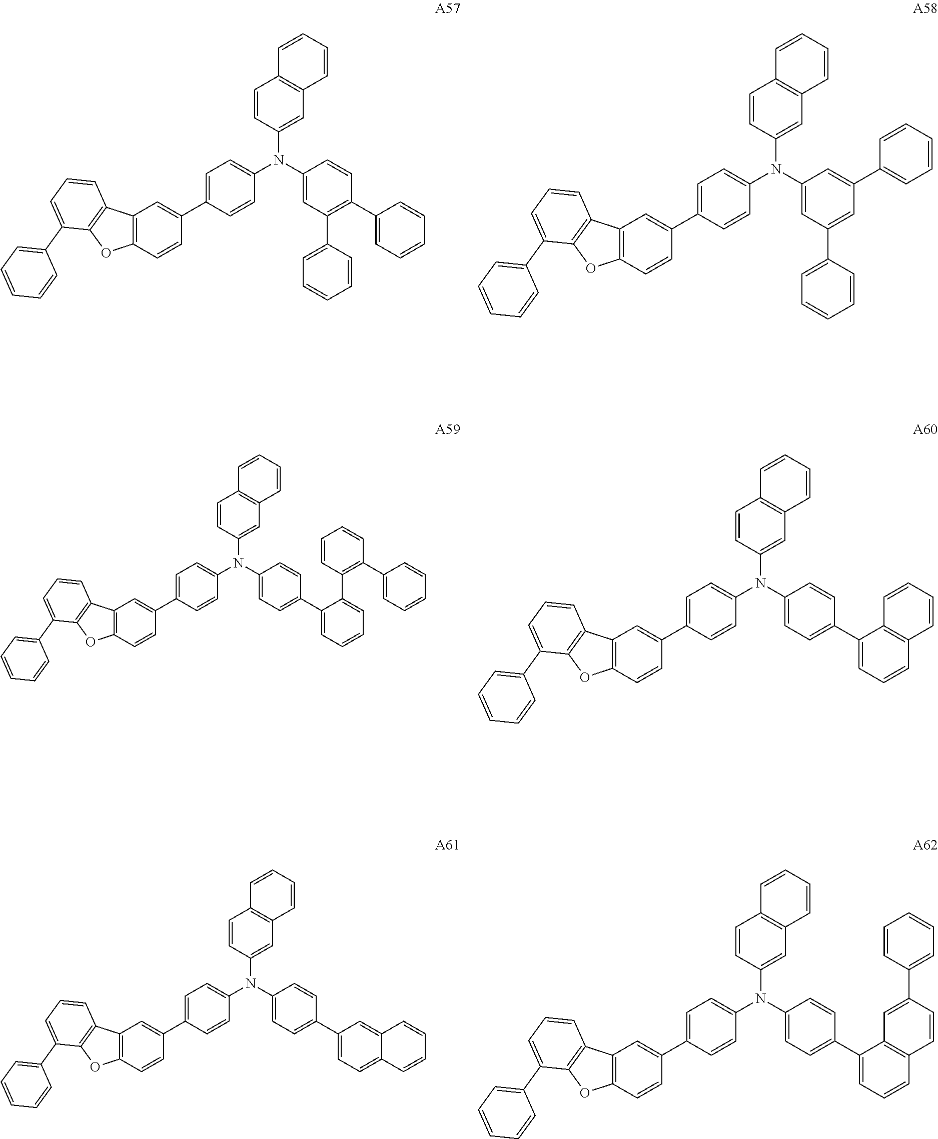 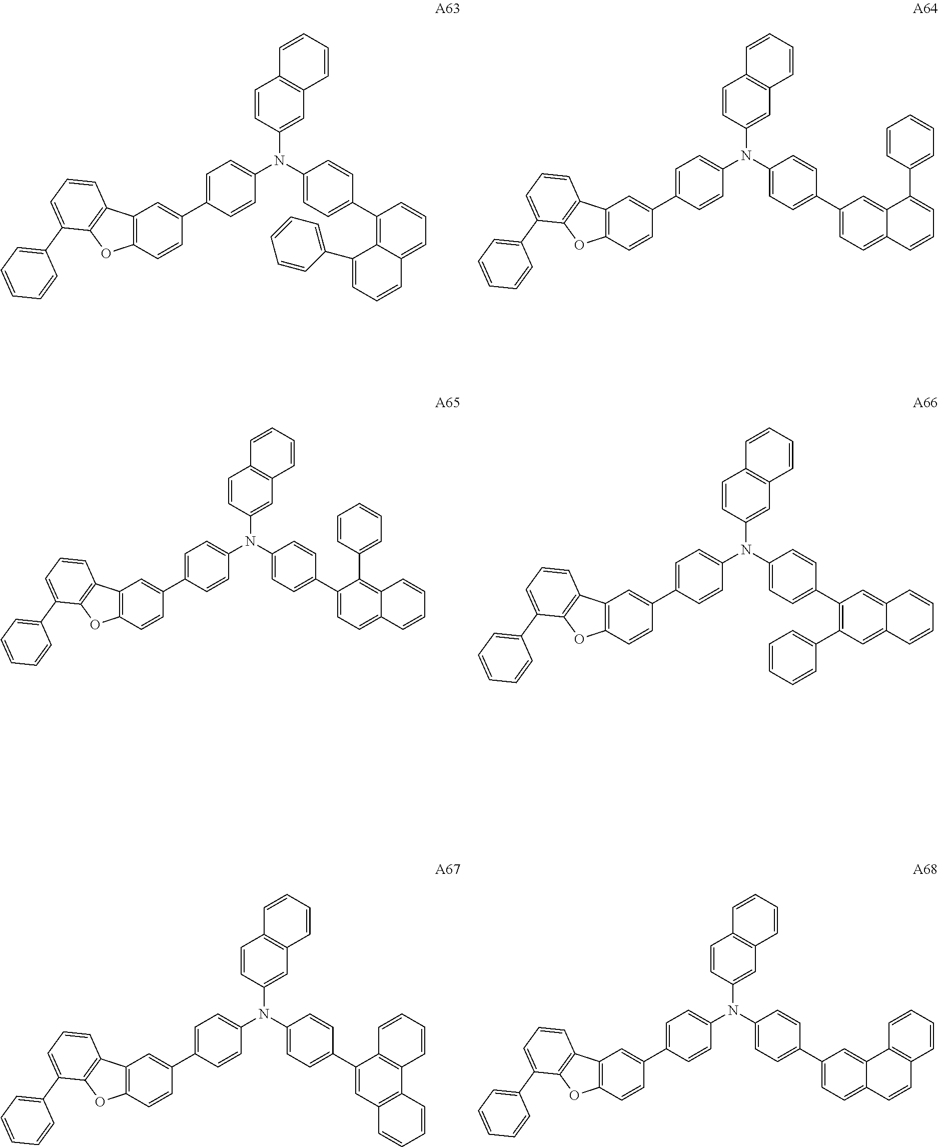 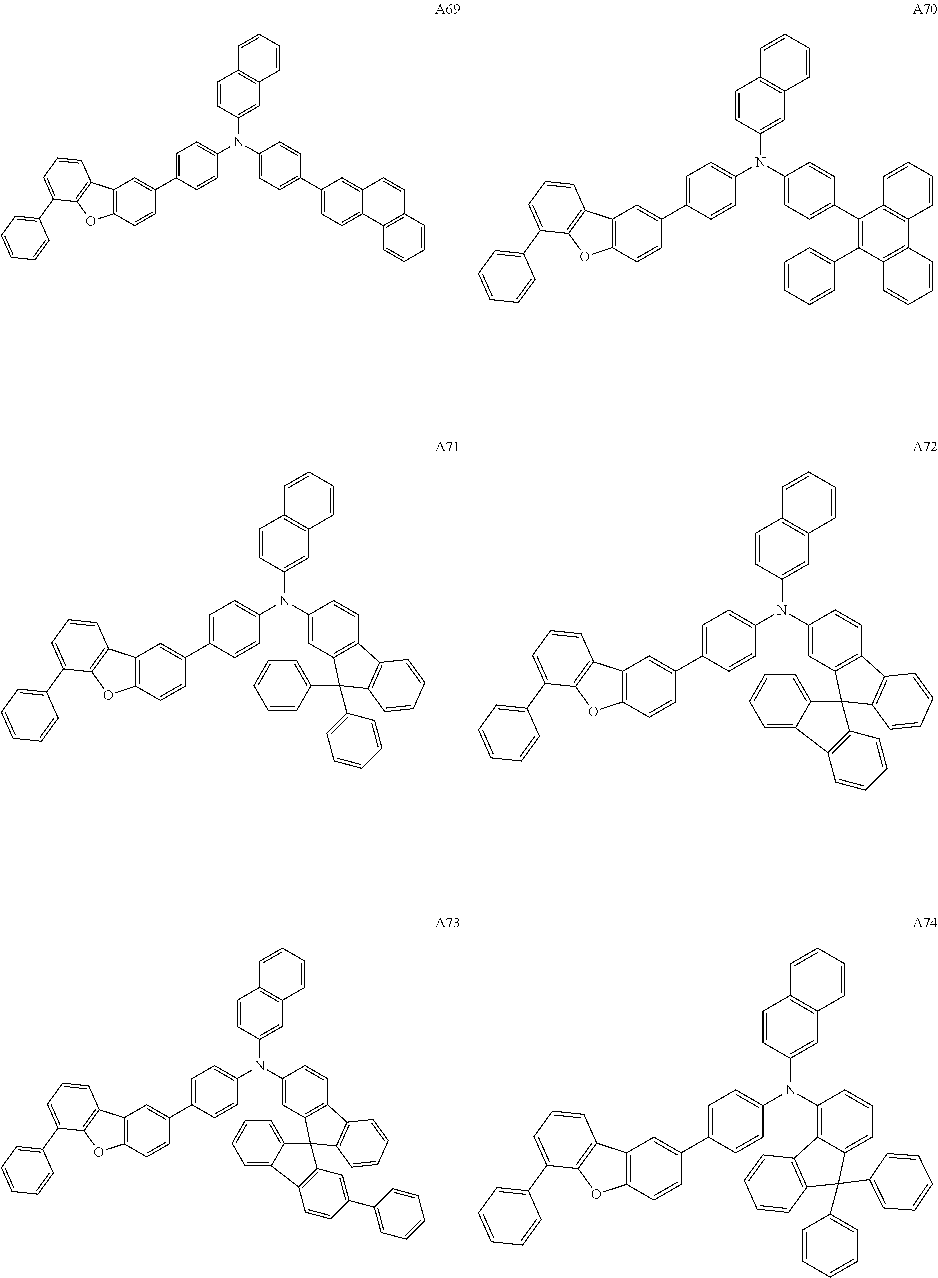 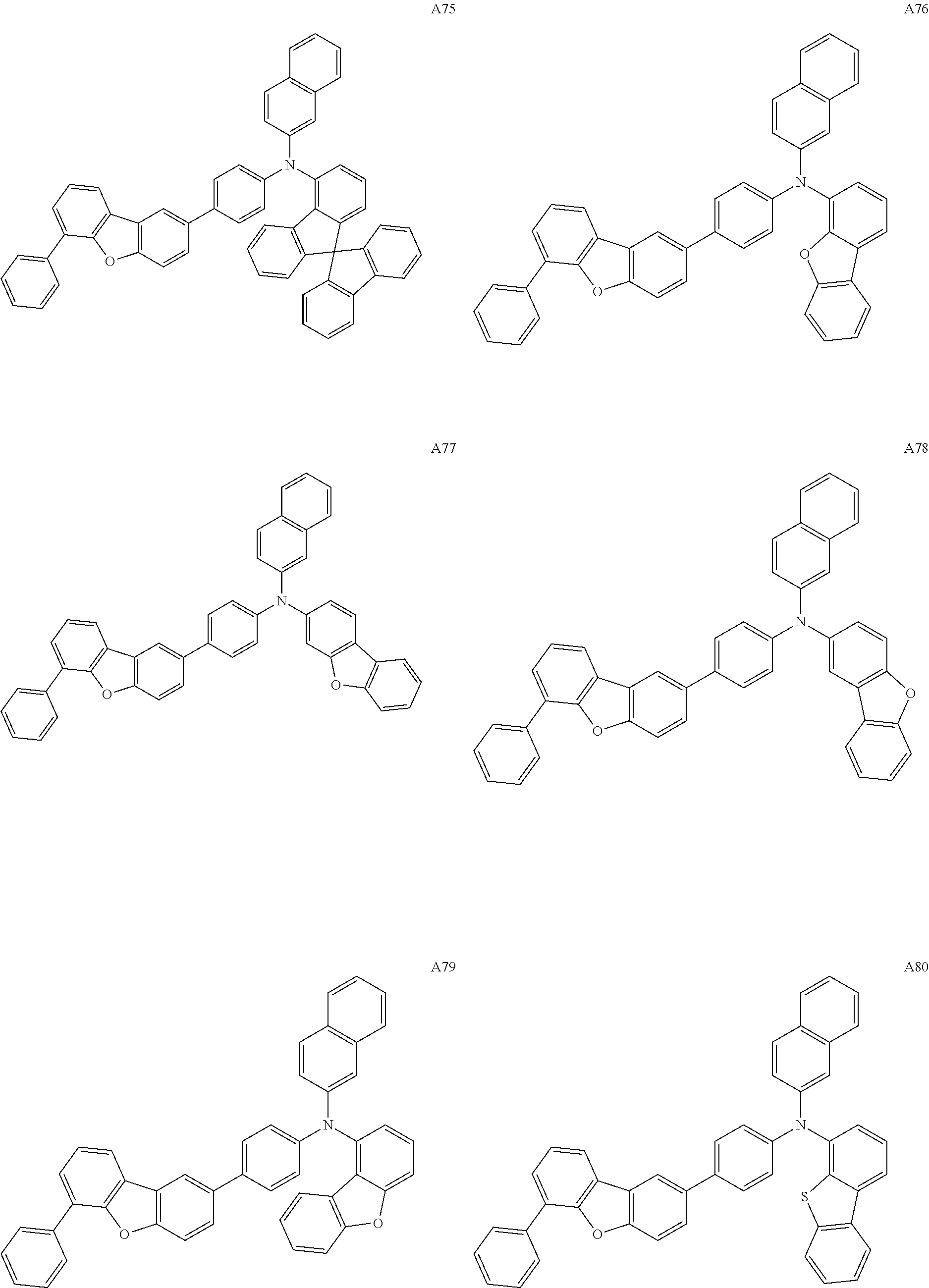 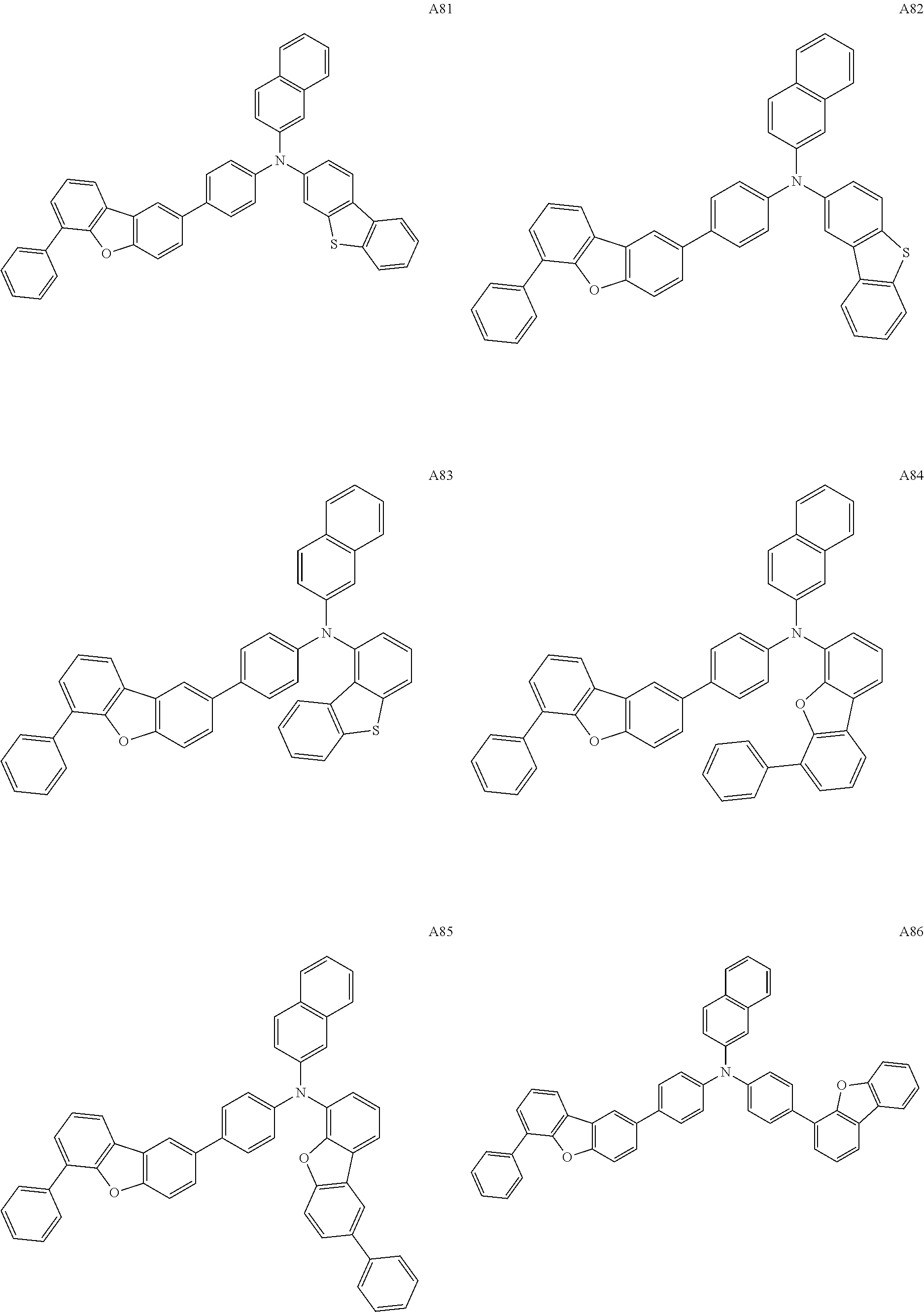 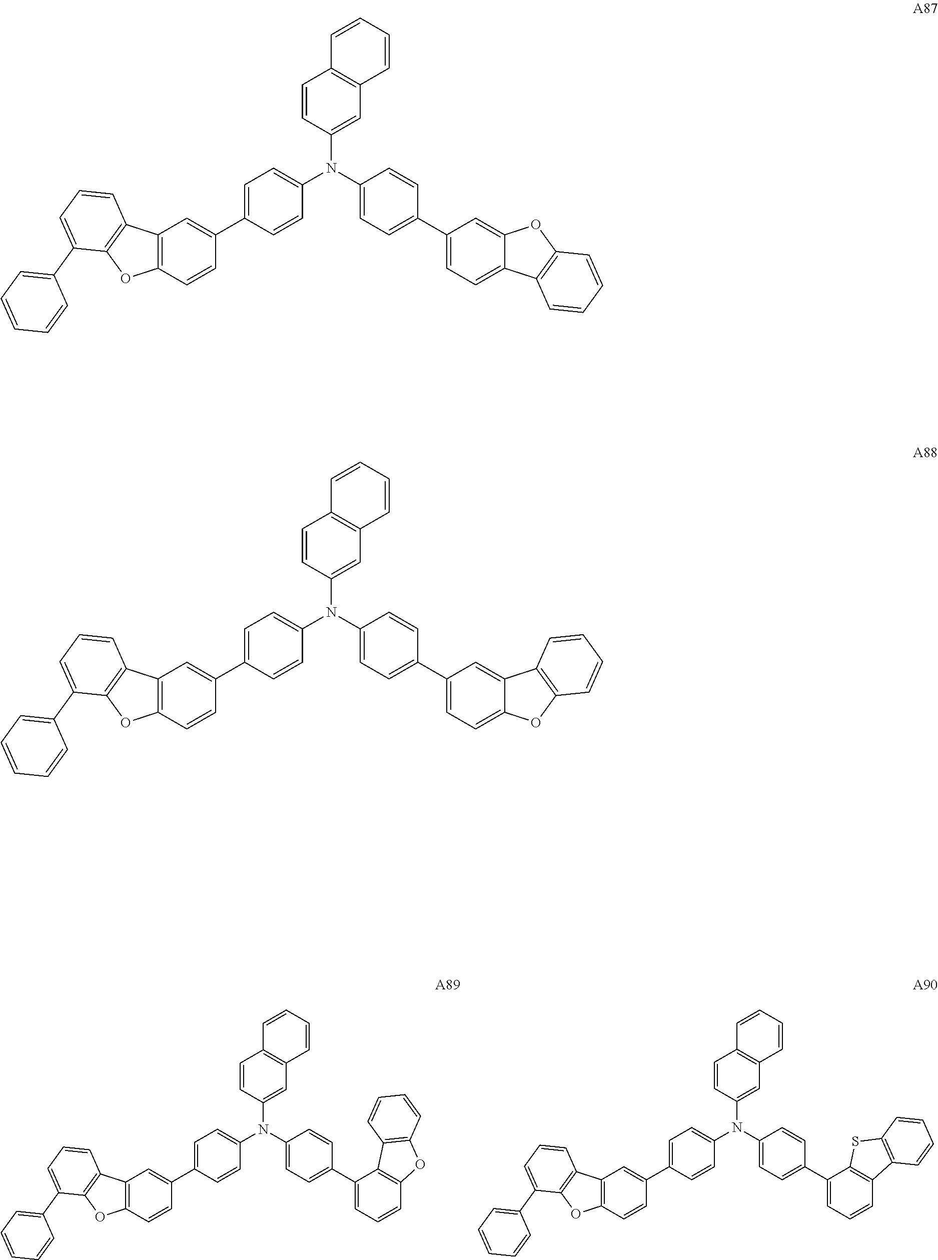 [SMILES: c1ccc(-c2cccc3c2oc2ccc(-c4ccc(N(c5ccc(-c6ccc7c(c6)oc6ccccc67)cc5)c5ccc6ccccc6c5)cc4)cc23)cc1 ; c1ccc(-c2cccc3c2oc2ccc(-c4ccc(N(c5ccc(-c6ccc7oc8ccccc8c7c6)cc5)c5ccc6ccccc6c5)cc4)cc23)cc1 ; c1ccc(-c2cccc3c2oc2ccc(-c4ccc(N(c5ccc(-c6cccc7oc8ccccc8c67)cc5)c5ccc6ccccc6c5)cc4)cc23)cc1 ; c1ccc(-c2cccc3c2oc2ccc(-c4ccc(N(c5ccc(-c6cccc7c6sc6ccccc67)cc5)c5ccc6ccccc6c5)cc4)cc23)cc1] 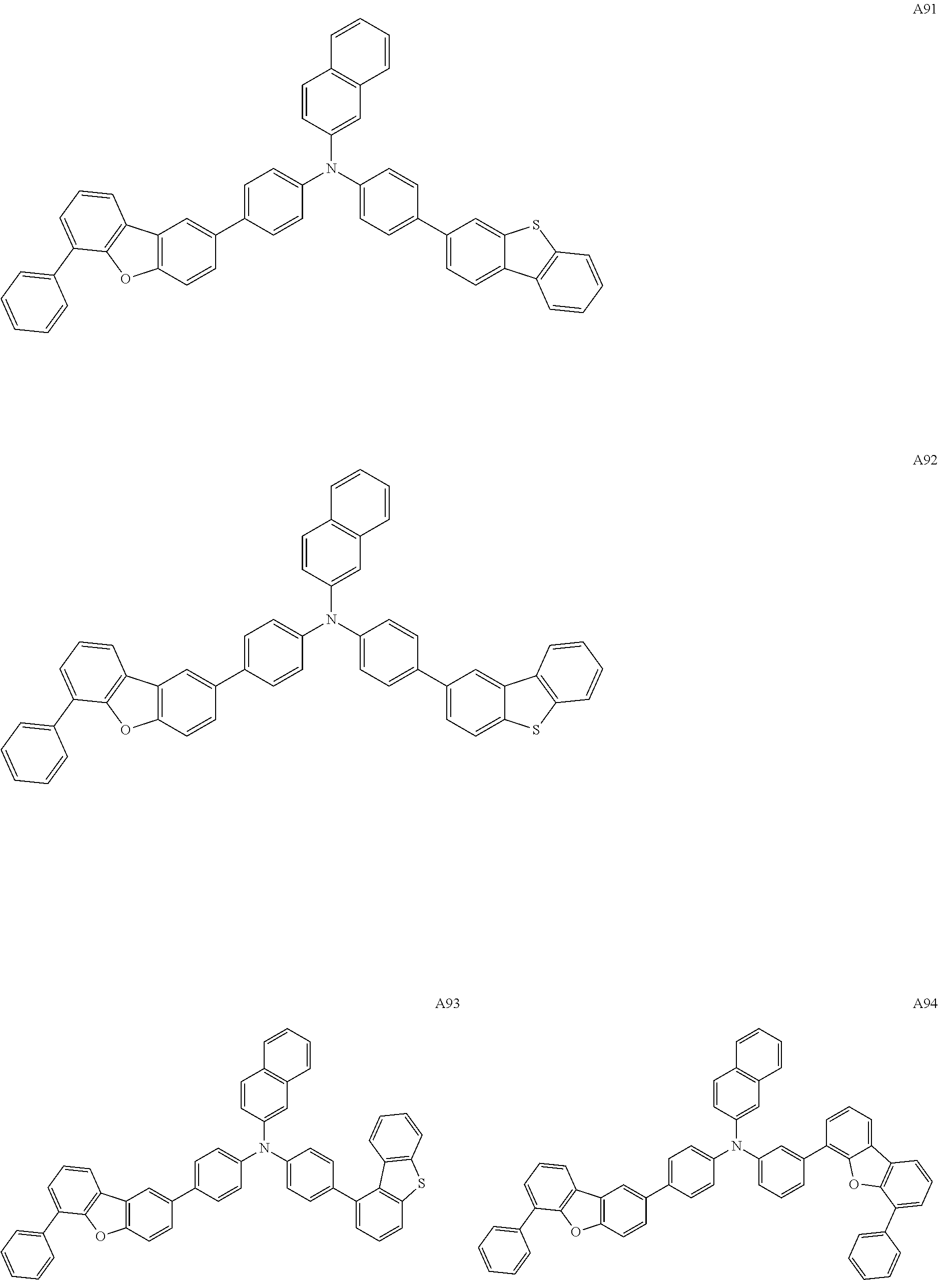 [SMILES: c1ccc(-c2cccc3c2oc2ccc(-c4ccc(N(c5ccc(-c6ccc7c(c6)sc6ccccc67)cc5)c5ccc6ccccc6c5)cc4)cc23)cc1 ; c1ccc(-c2cccc3c2oc2ccc(-c4ccc(N(c5ccc(-c6ccc7sc8ccccc8c7c6)cc5)c5ccc6ccccc6c5)cc4)cc23)cc1 ; c1ccc(-c2cccc3c2oc2ccc(-c4ccc(N(c5ccc(-c6cccc7sc8ccccc8c67)cc5)c5ccc6ccccc6c5)cc4)cc23)cc1 ; c1ccc(-c2cccc3c2oc2ccc(-c4ccc(N(c5cccc(-c6cccc7c6oc6c(-c8ccccc8)cccc67)c5)c5ccc6ccccc6c5)cc4)cc23)cc1] 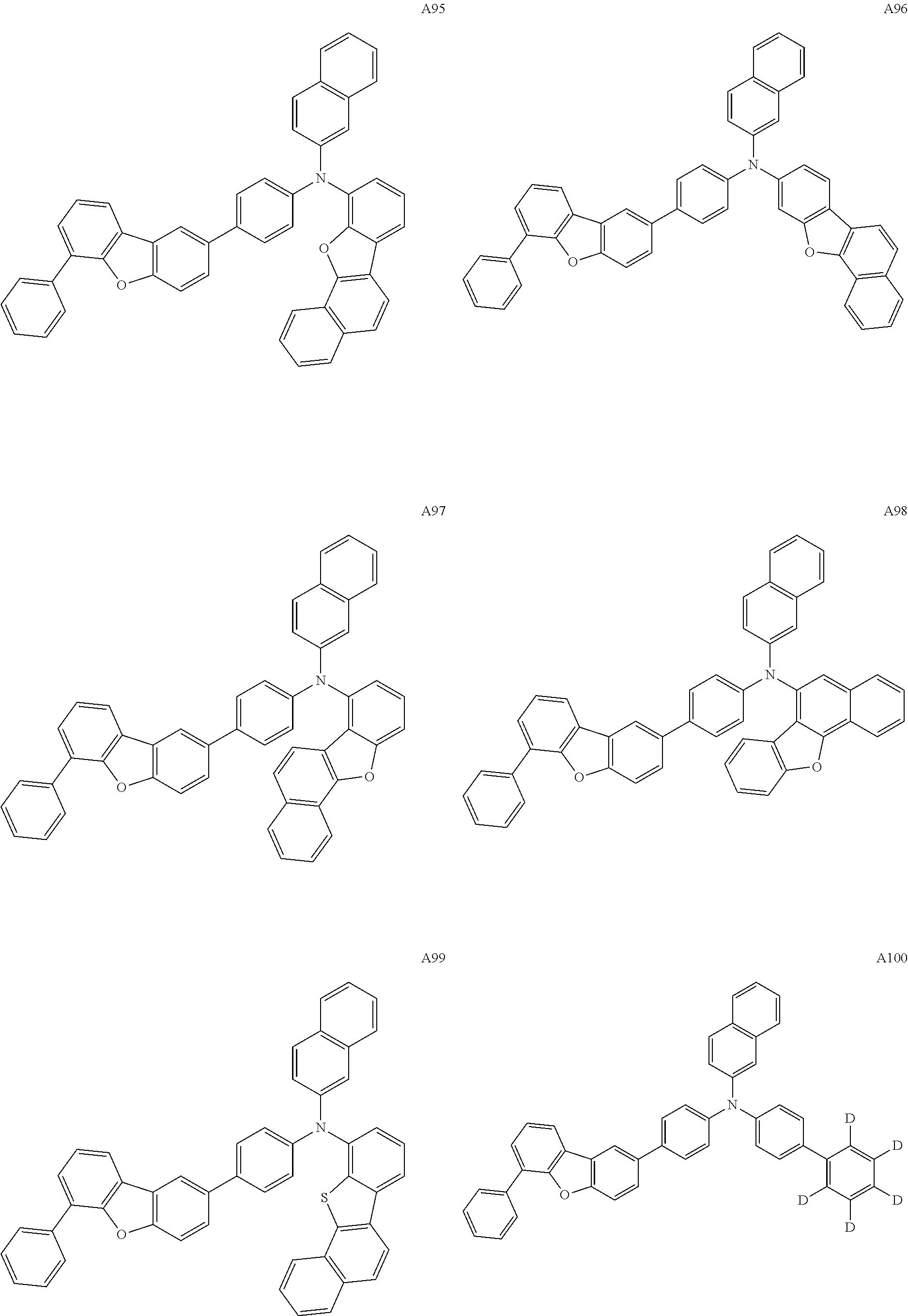 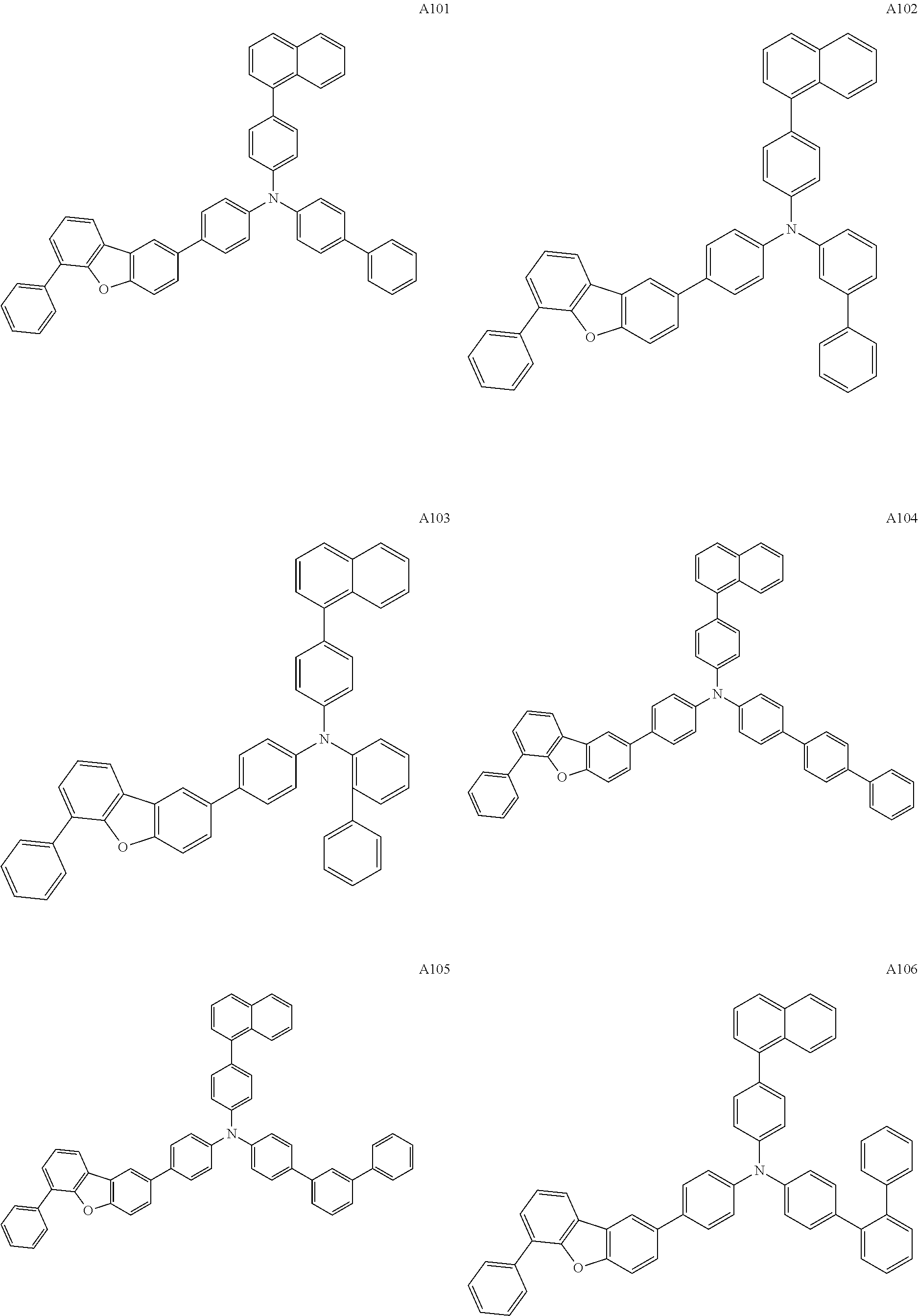 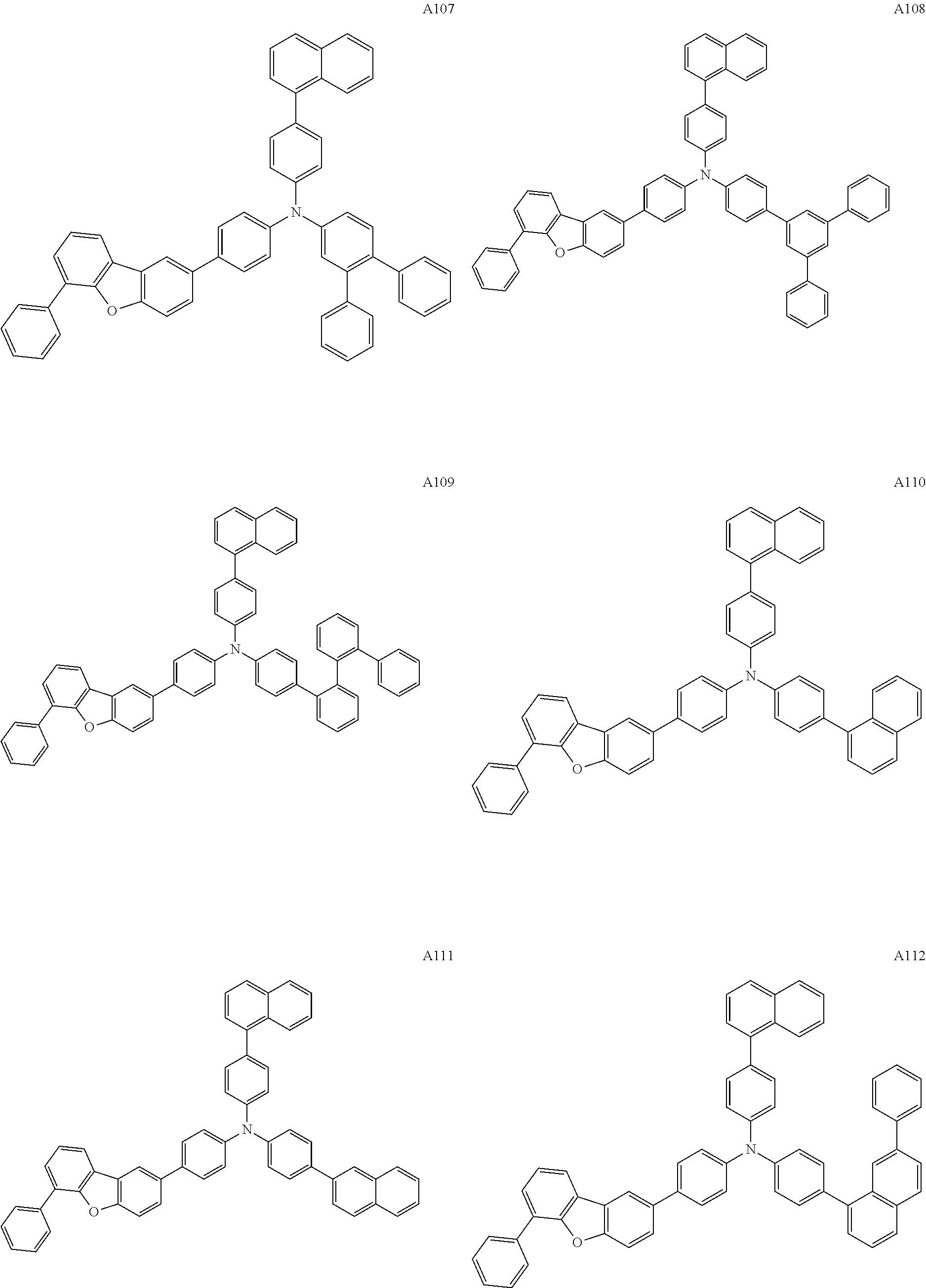 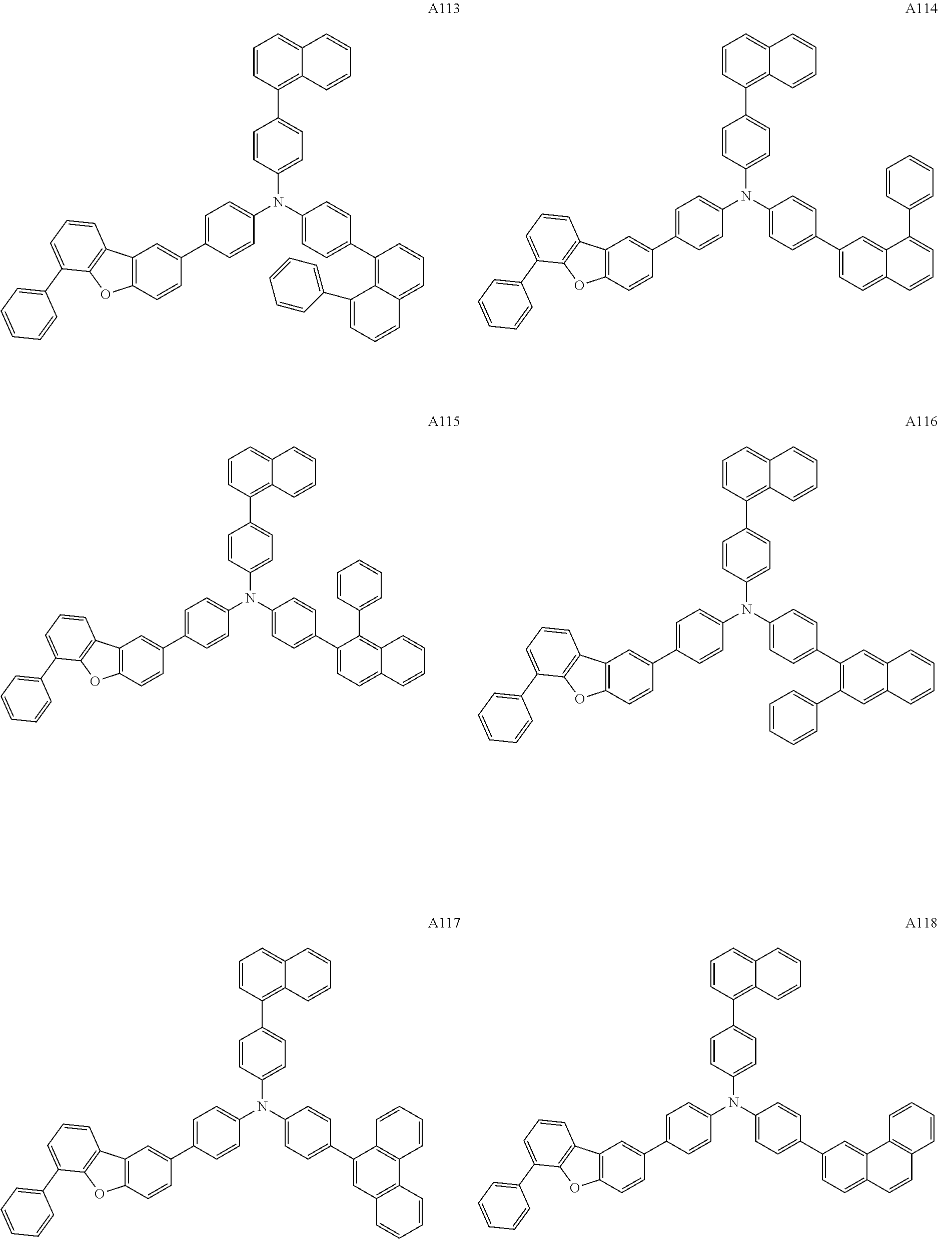 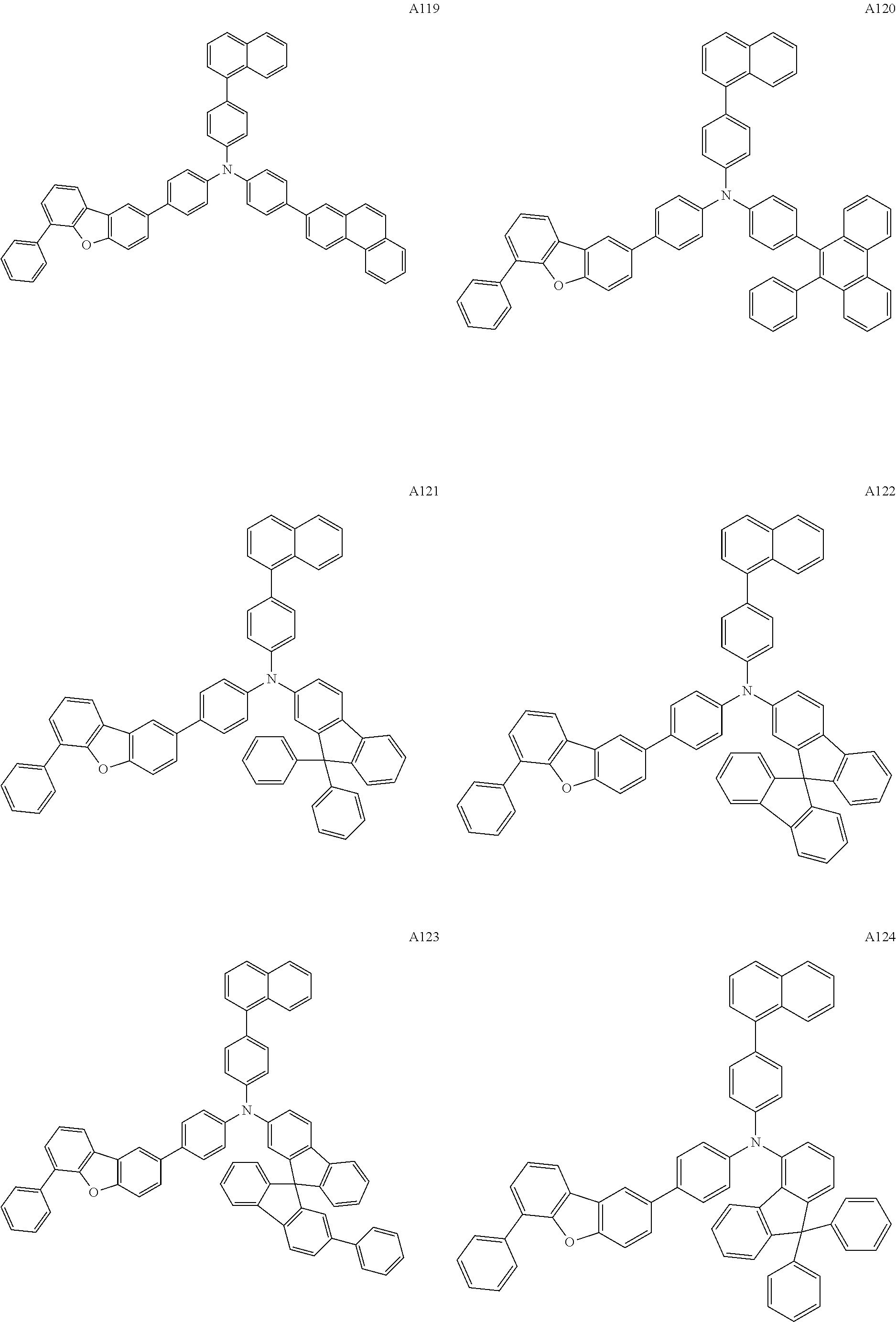 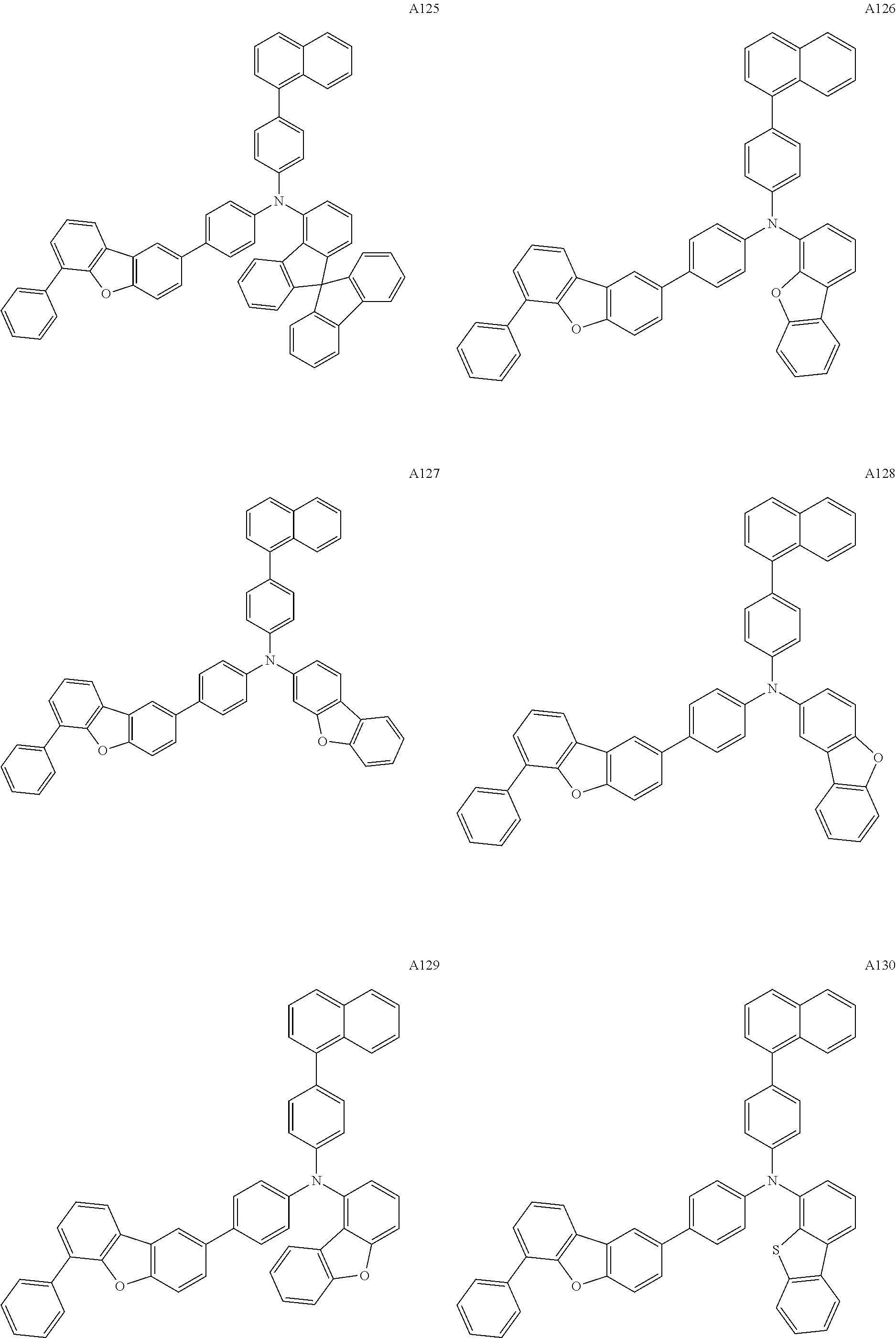 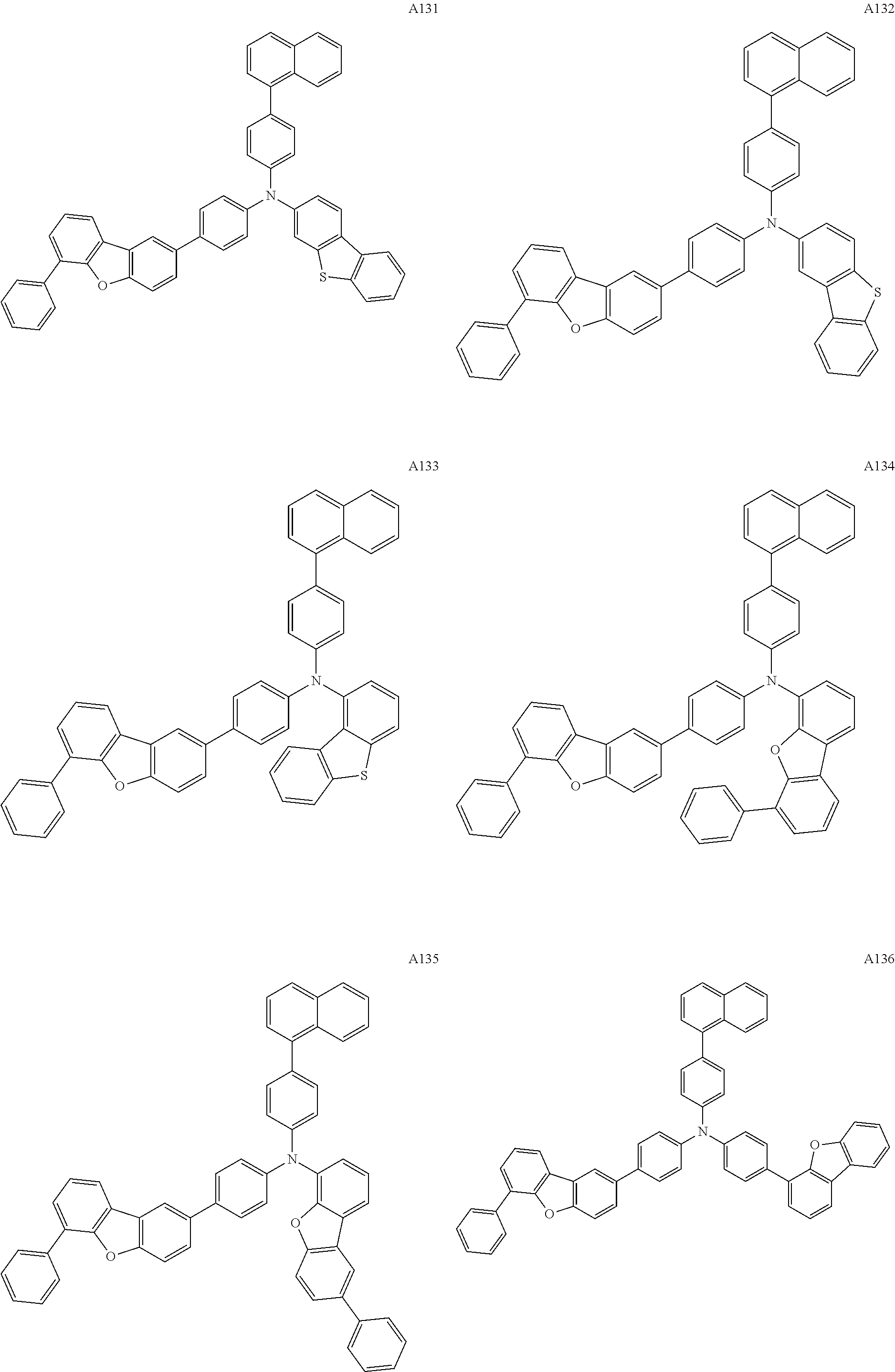 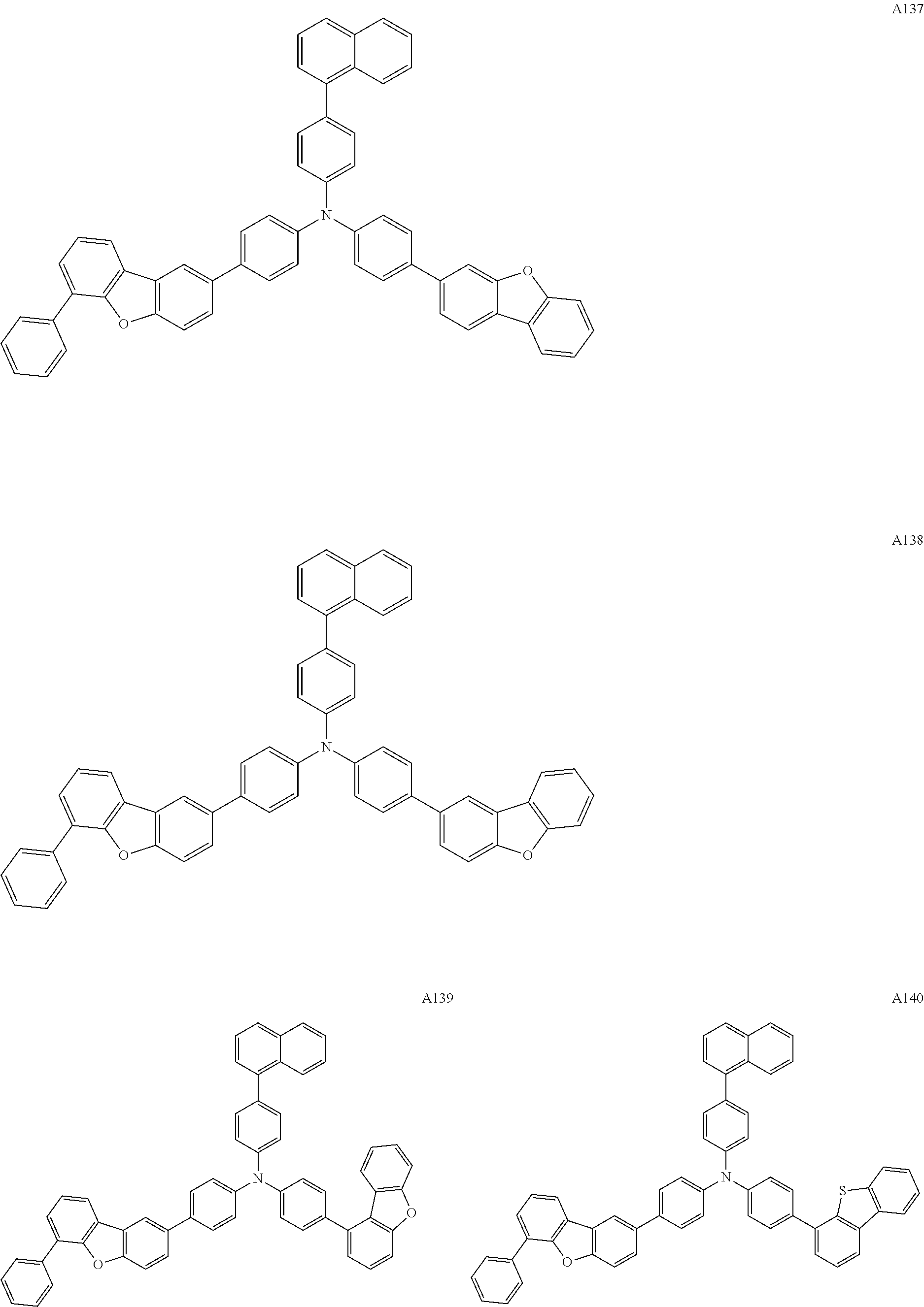 [SMILES: c1ccc(-c2cccc3c2oc2ccc(-c4ccc(N(c5ccc(-c6ccc7c(c6)oc6ccccc67)cc5)c5ccc(-c6cccc7ccccc67)cc5)cc4)cc23)cc1 ; c1ccc(-c2cccc3c2oc2ccc(-c4ccc(N(c5ccc(-c6ccc7oc8ccccc8c7c6)cc5)c5ccc(-c6cccc7ccccc67)cc5)cc4)cc23)cc1 ; c1ccc(-c2cccc3c2oc2ccc(-c4ccc(N(c5ccc(-c6cccc7ccccc67)cc5)c5ccc(-c6cccc7oc8ccccc8c67)cc5)cc4)cc23)cc1 ; c1ccc(-c2cccc3c2oc2ccc(-c4ccc(N(c5ccc(-c6cccc7ccccc67)cc5)c5ccc(-c6cccc7c6sc6ccccc67)cc5)cc4)cc23)cc1] 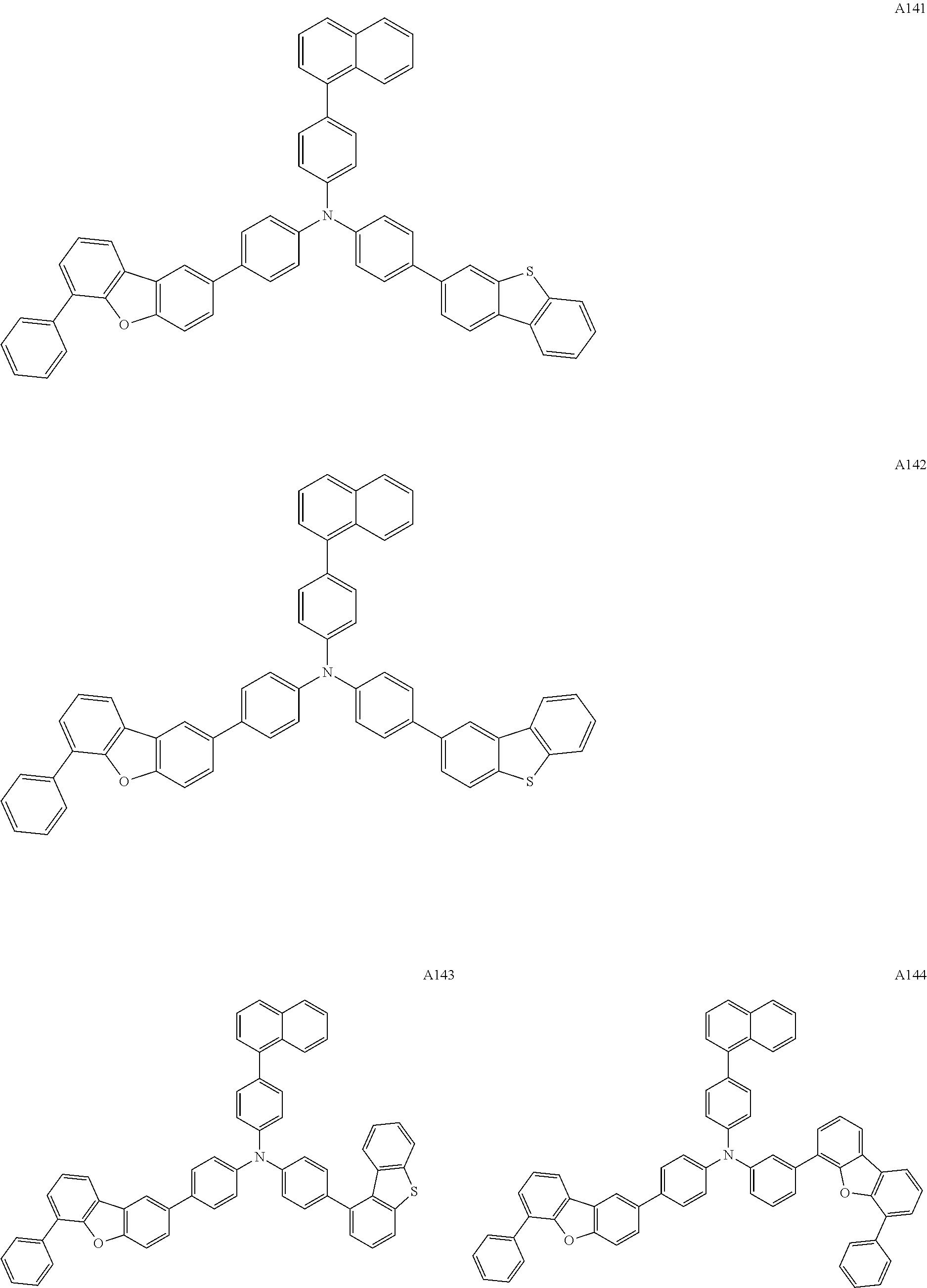 [SMILES: c1ccc(-c2cccc3c2oc2ccc(-c4ccc(N(c5ccc(-c6ccc7c(c6)sc6ccccc67)cc5)c5ccc(-c6cccc7ccccc67)cc5)cc4)cc23)cc1 ; c1ccc(-c2cccc3c2oc2ccc(-c4ccc(N(c5ccc(-c6ccc7sc8ccccc8c7c6)cc5)c5ccc(-c6cccc7ccccc67)cc5)cc4)cc23)cc1 ; c1ccc(-c2cccc3c2oc2ccc(-c4ccc(N(c5ccc(-c6cccc7ccccc67)cc5)c5ccc(-c6cccc7sc8ccccc8c67)cc5)cc4)cc23)cc1 ; c1ccc(-c2cccc3c2oc2ccc(-c4ccc(N(c5ccc(-c6cccc7ccccc67)cc5)c5cccc(-c6cccc7c6oc6c(-c8ccccc8)cccc67)c5)cc4)cc23)cc1] 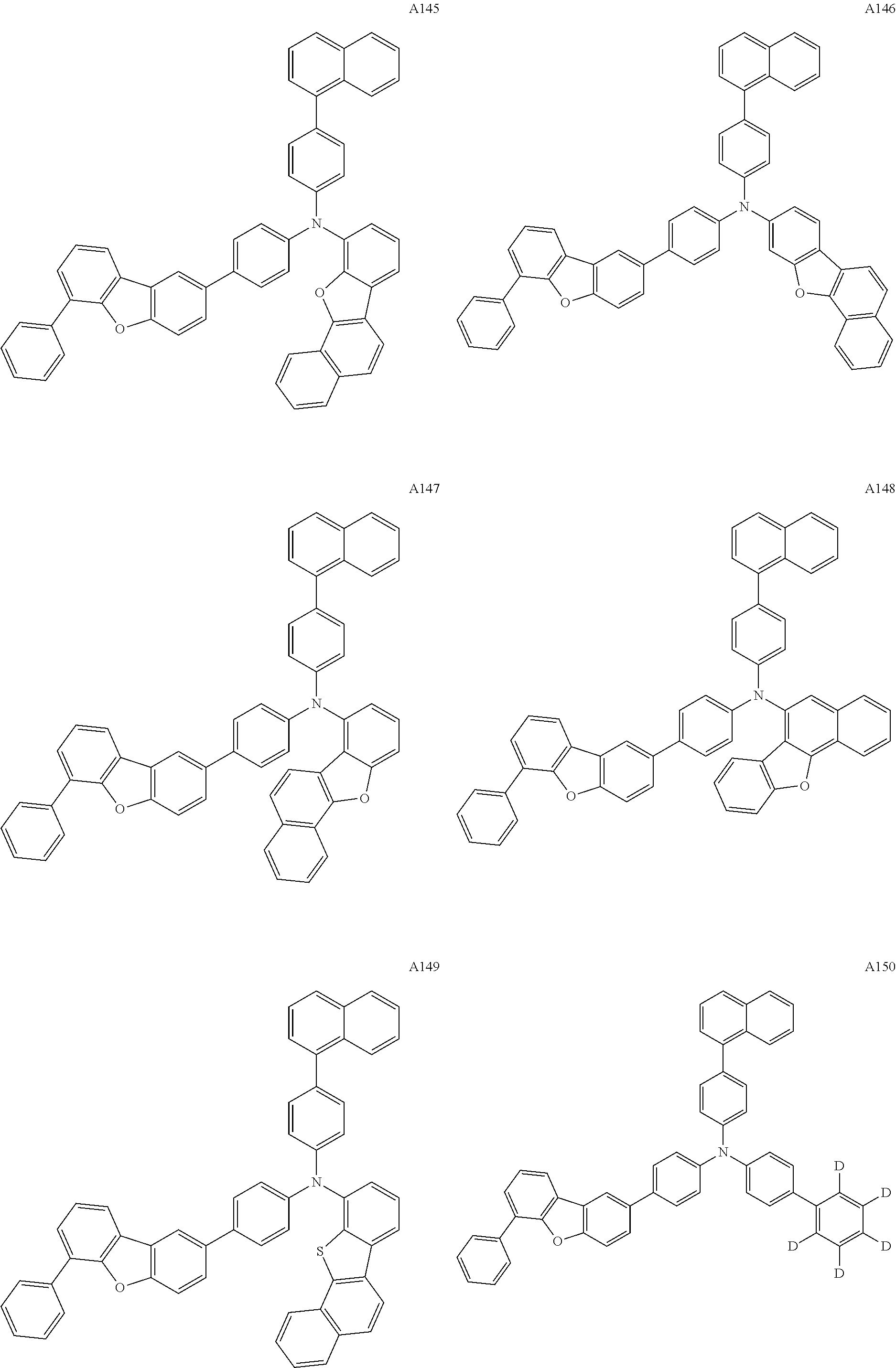 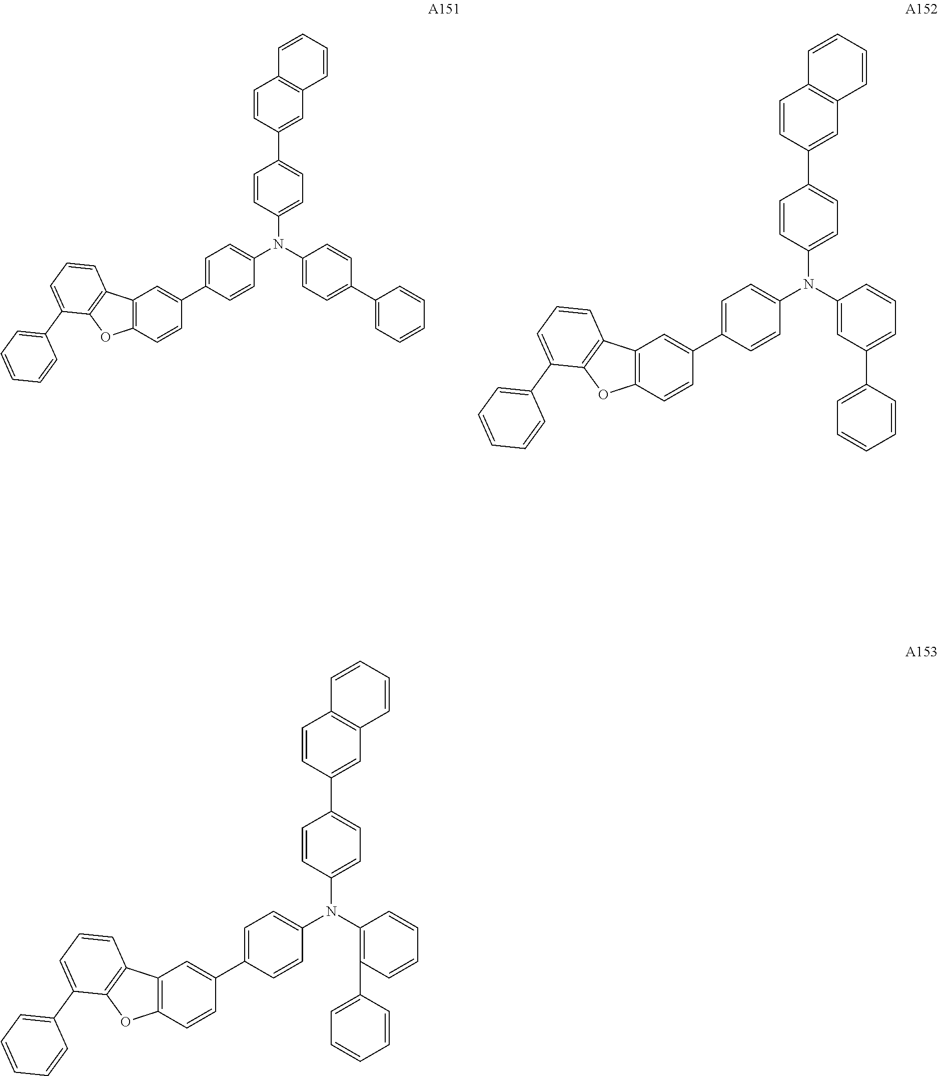 [SMILES: c1ccc(-c2ccc(N(c3ccc(-c4ccc5ccccc5c4)cc3)c3ccc(-c4ccc5oc6c(-c7ccccc7)cccc6c5c4)cc3)cc2)cc1 ; c1ccc(-c2cccc(N(c3ccc(-c4ccc5ccccc5c4)cc3)c3ccc(-c4ccc5oc6c(-c7ccccc7)cccc6c5c4)cc3)c2)cc1 ; c1ccc(-c2ccccc2N(c2ccc(-c3ccc4ccccc4c3)cc2)c2ccc(-c3ccc4oc5c(-c6ccccc6)cccc5c4c3)cc2)cc1] 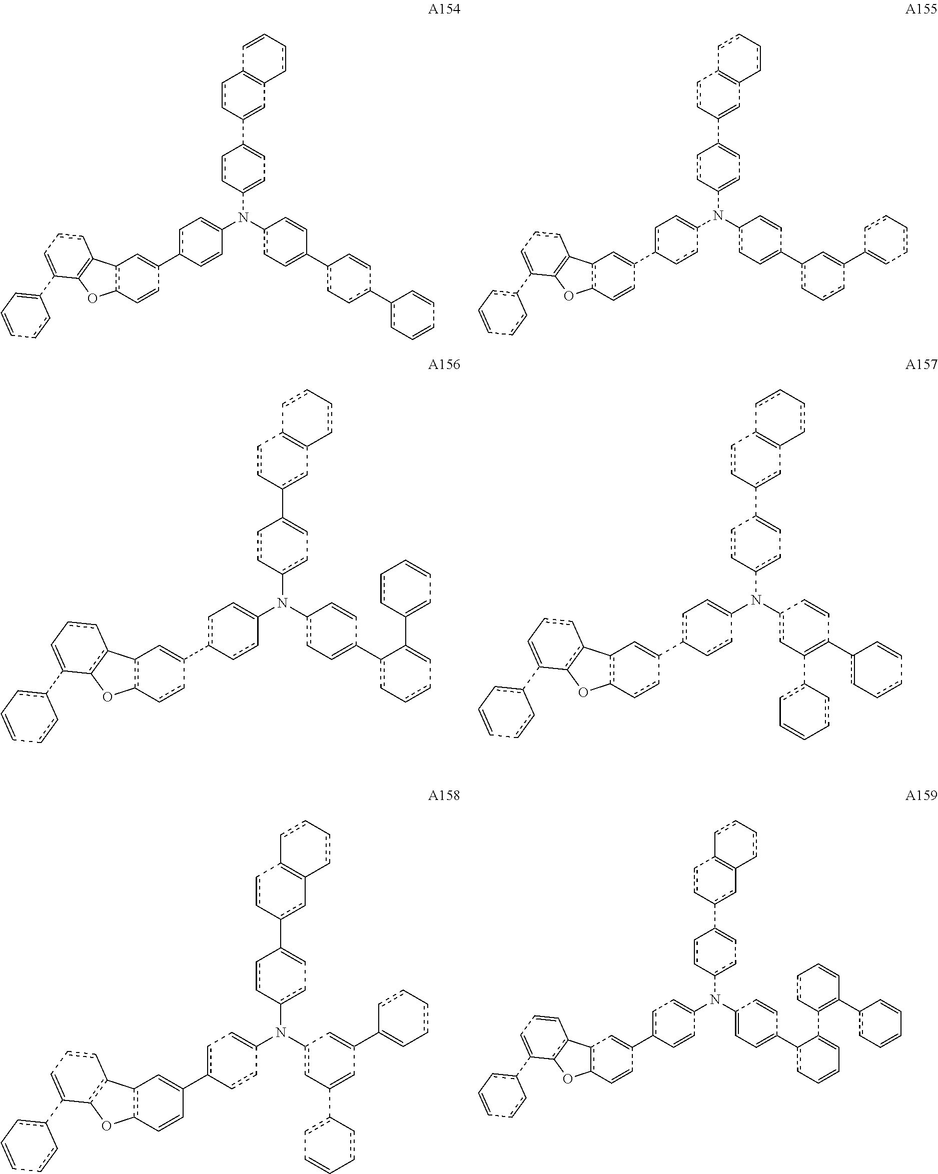 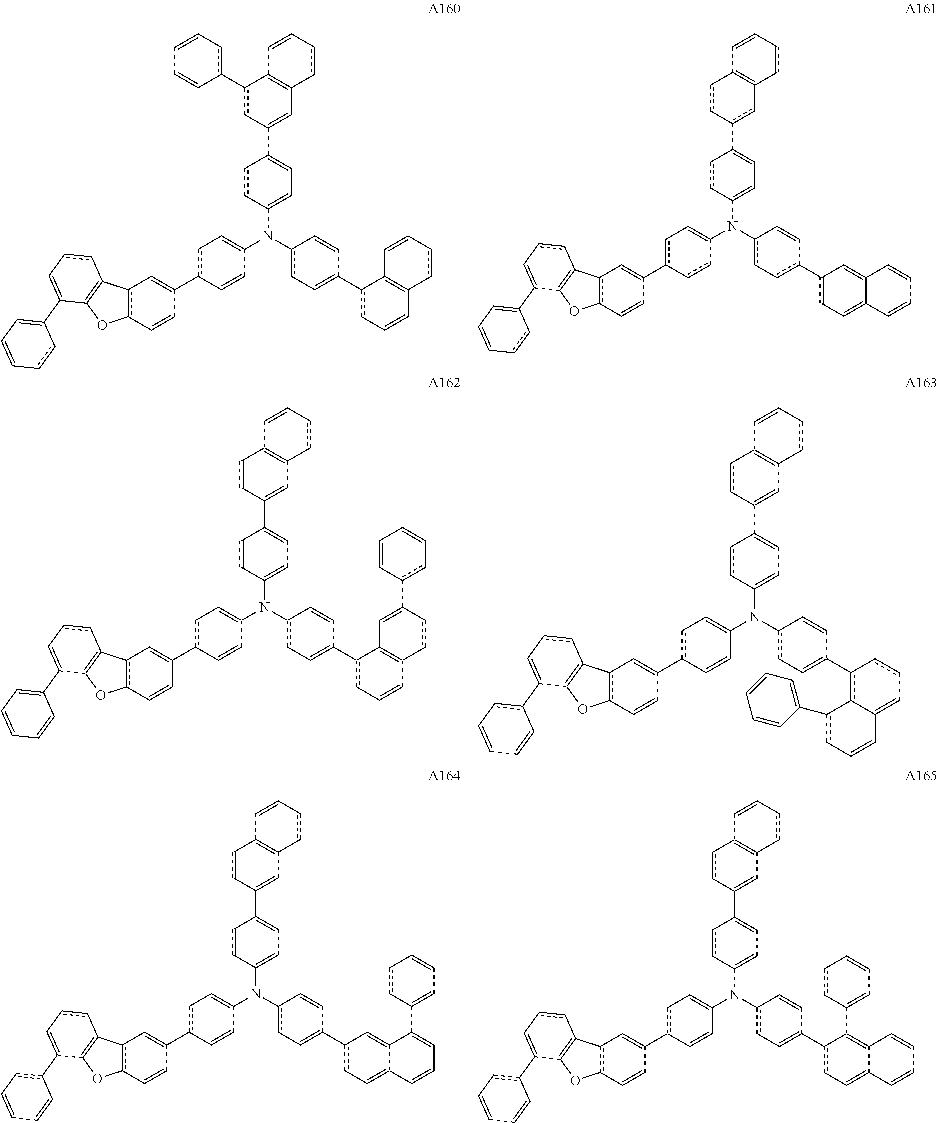 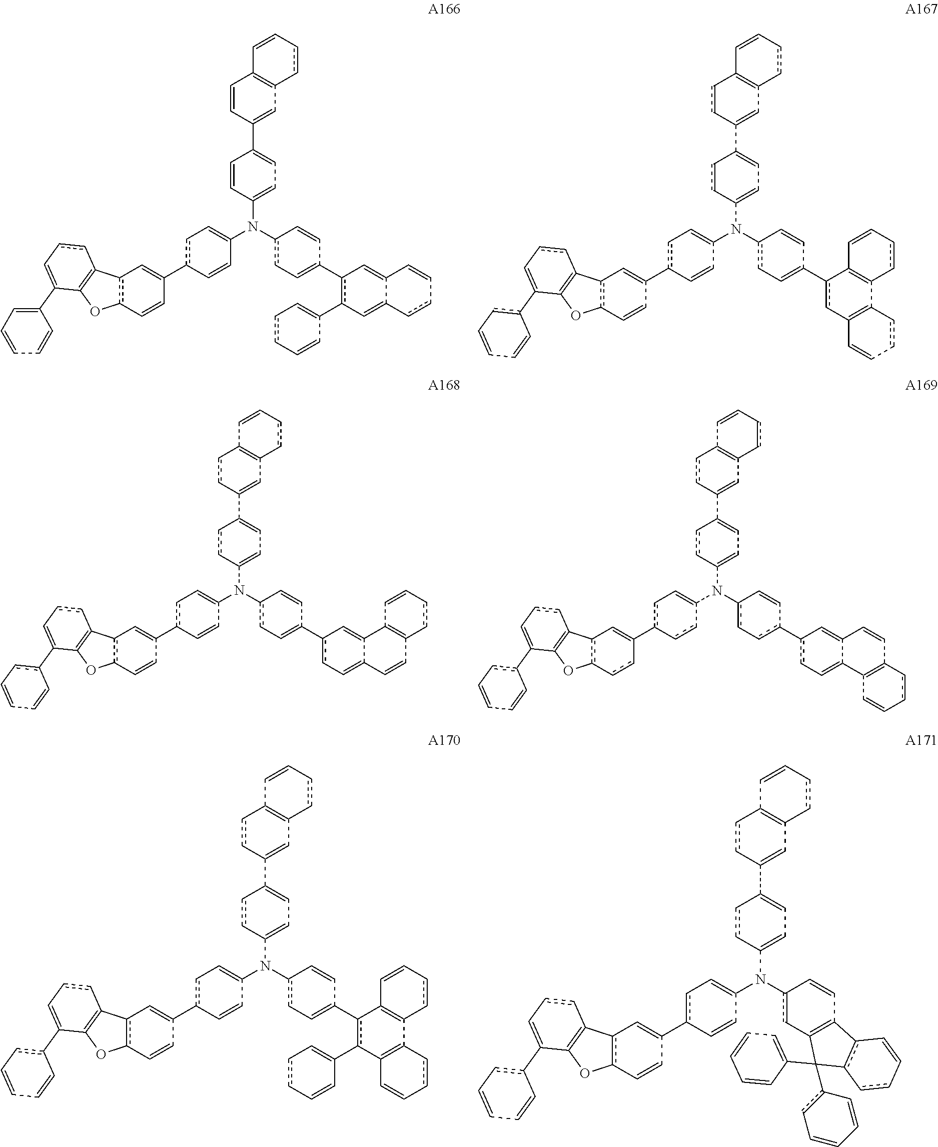 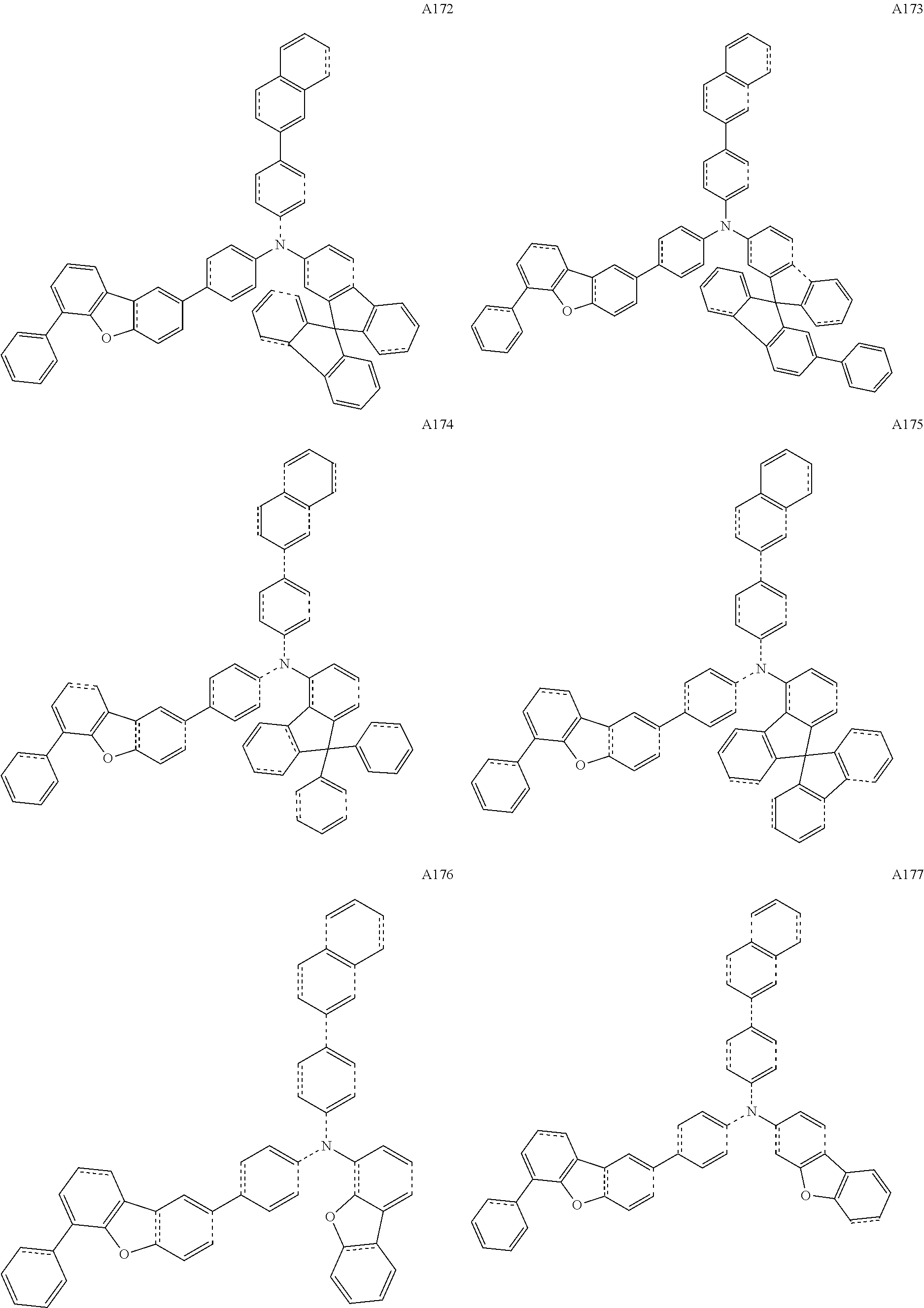 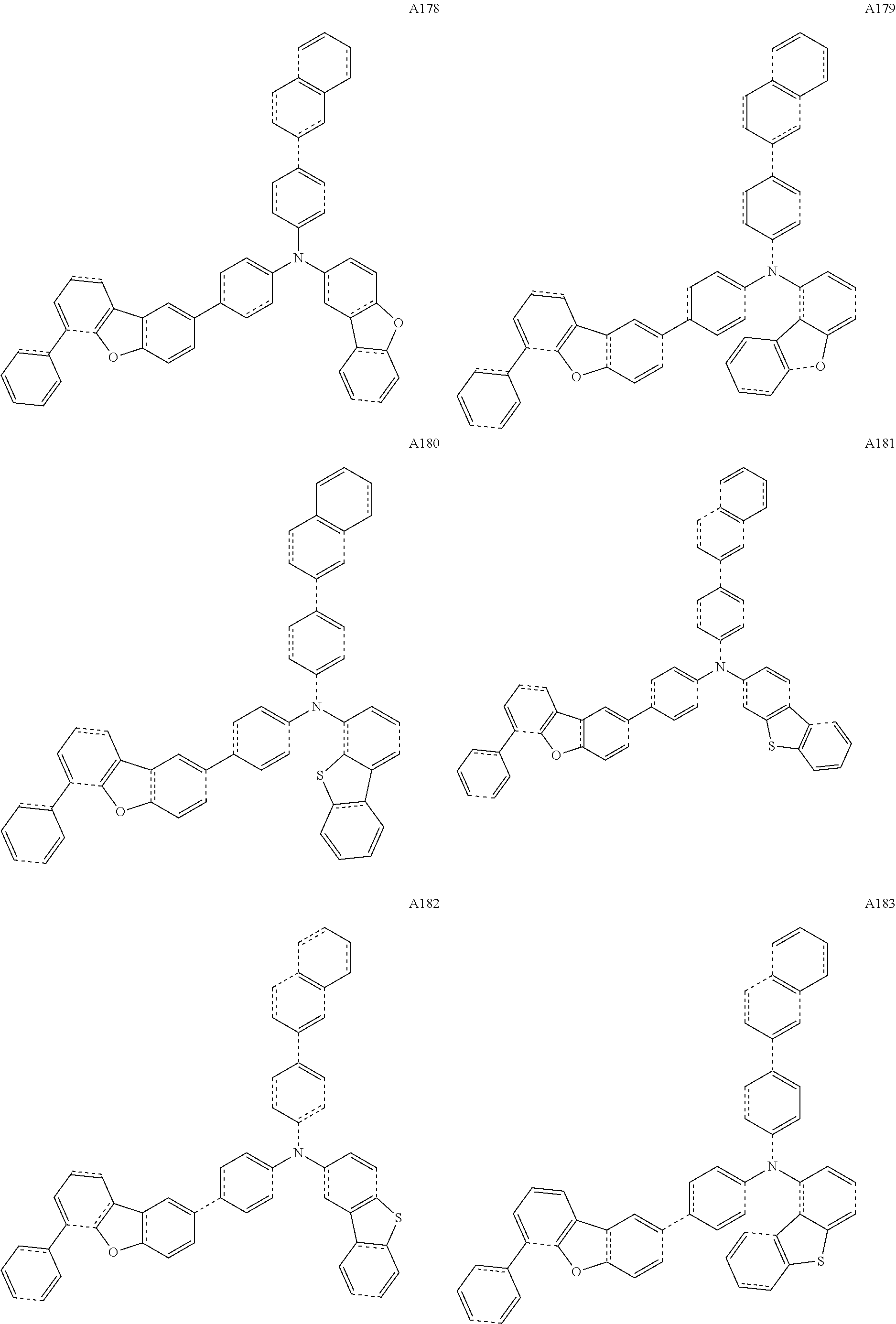 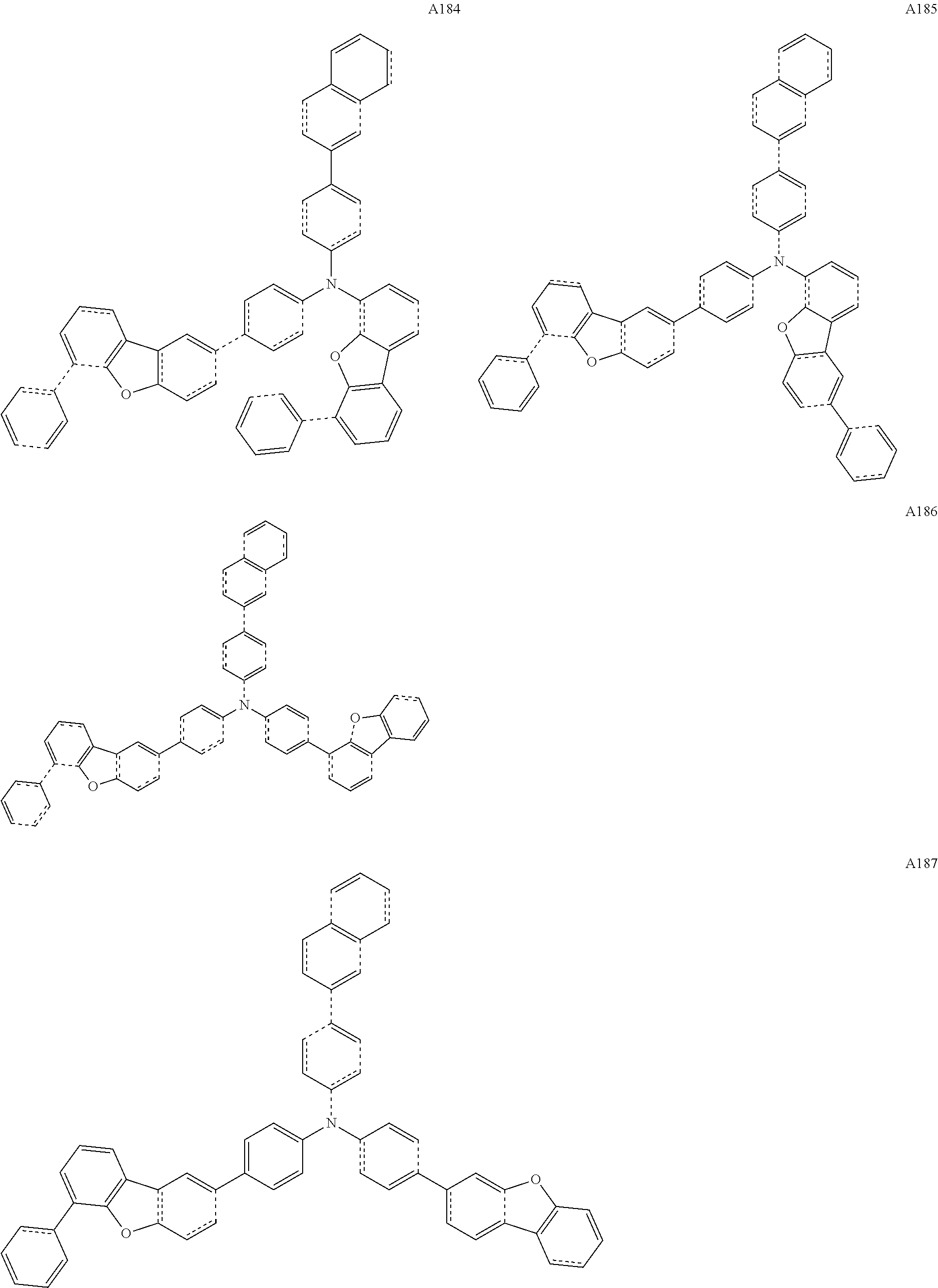 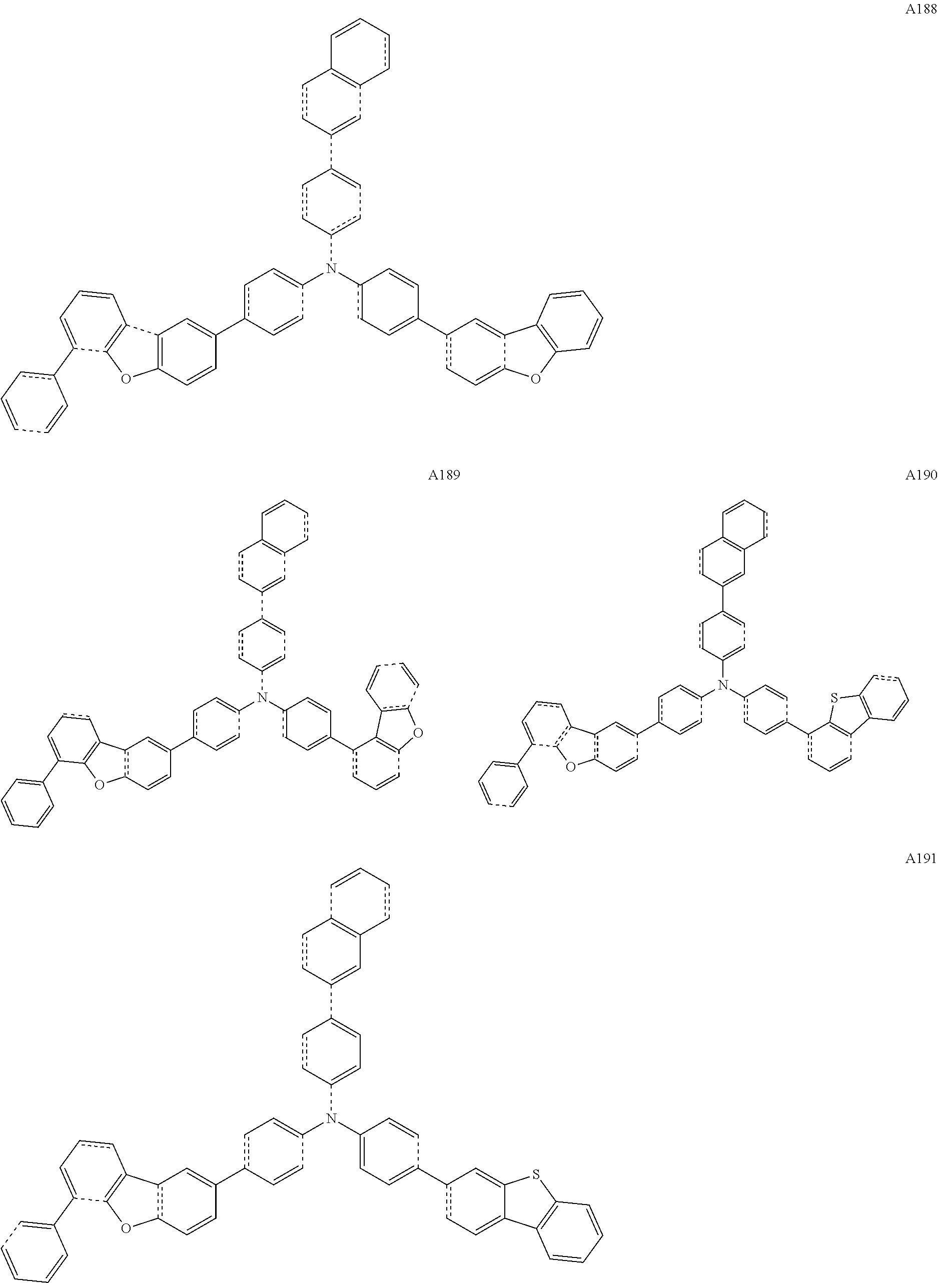 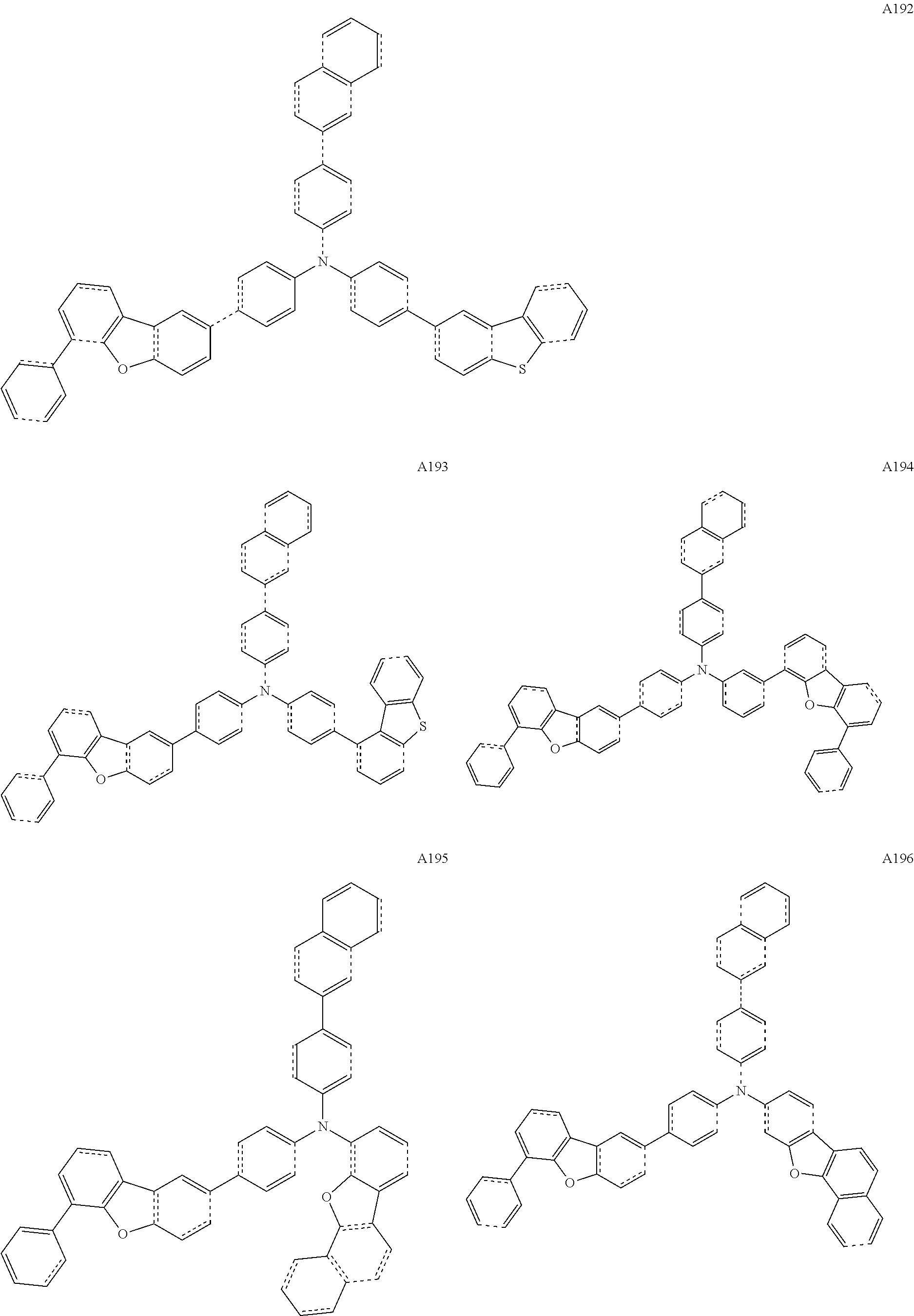 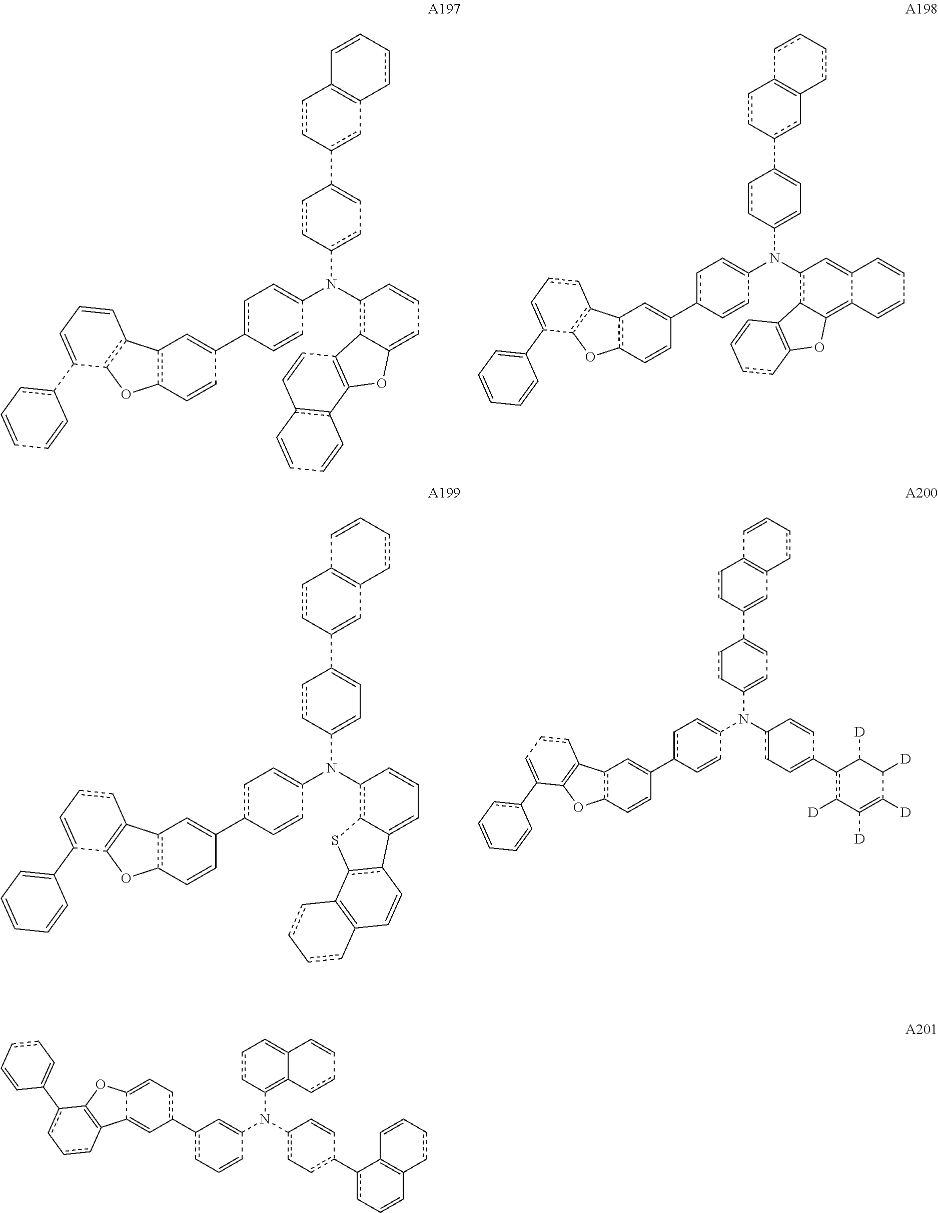 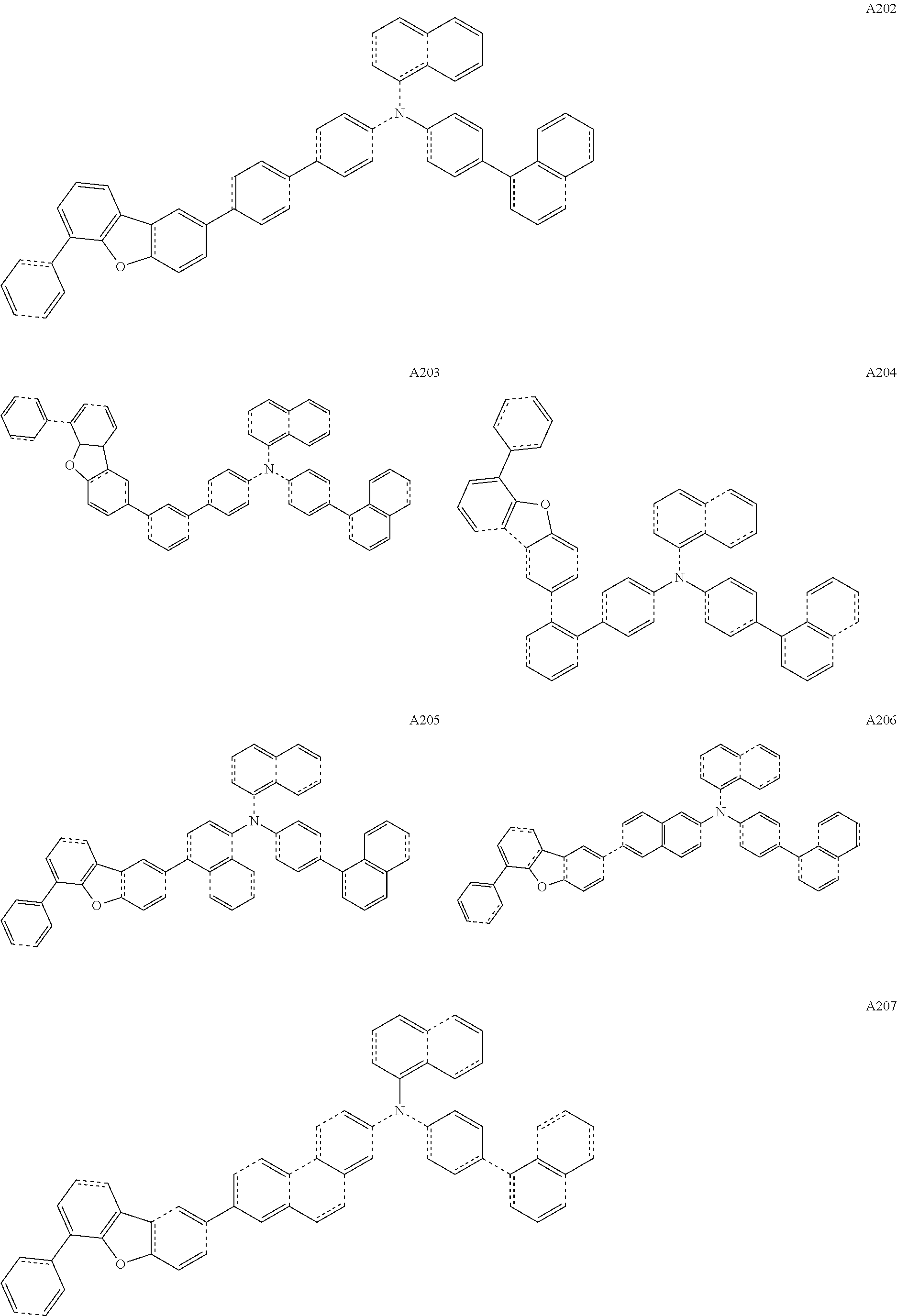 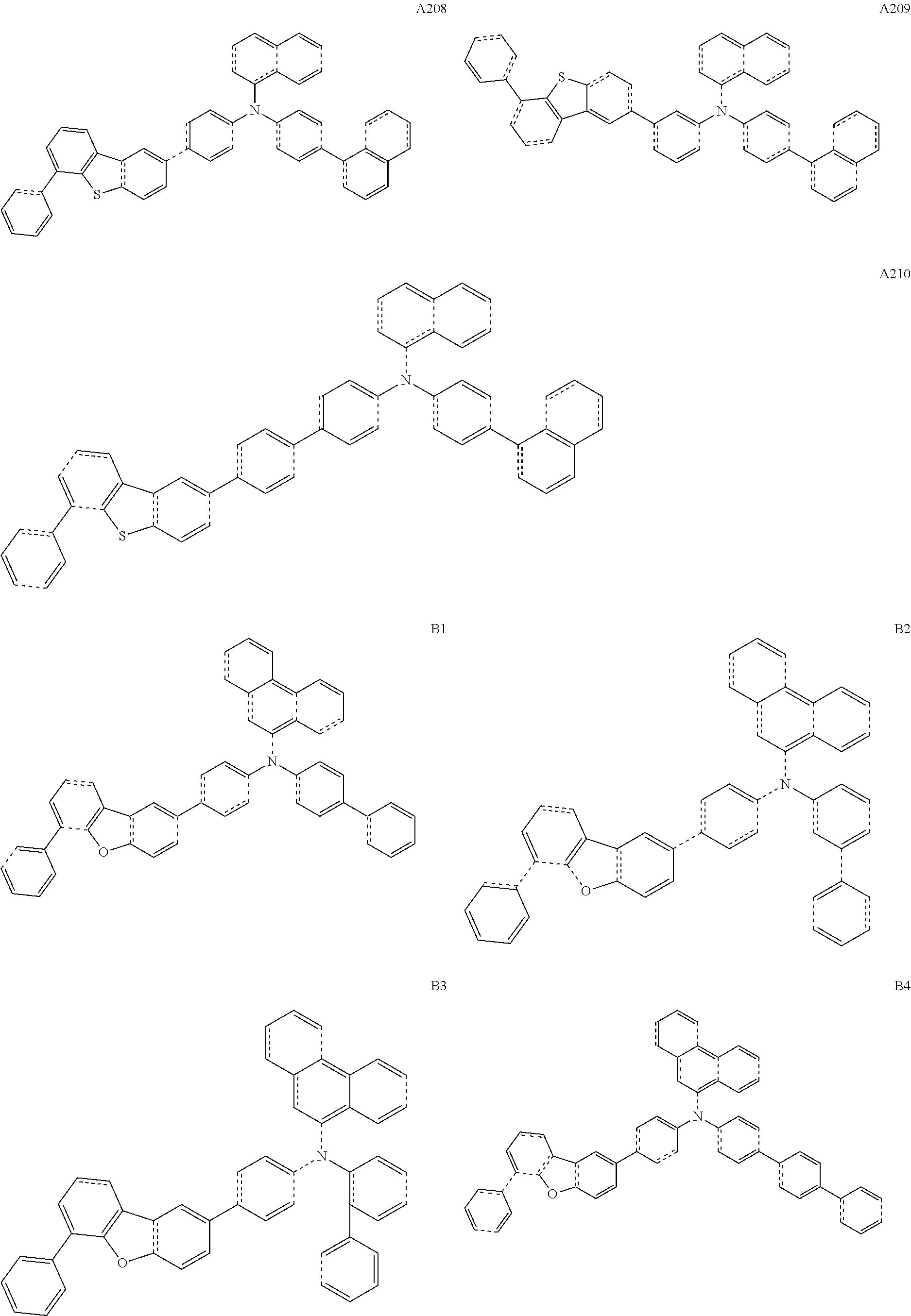 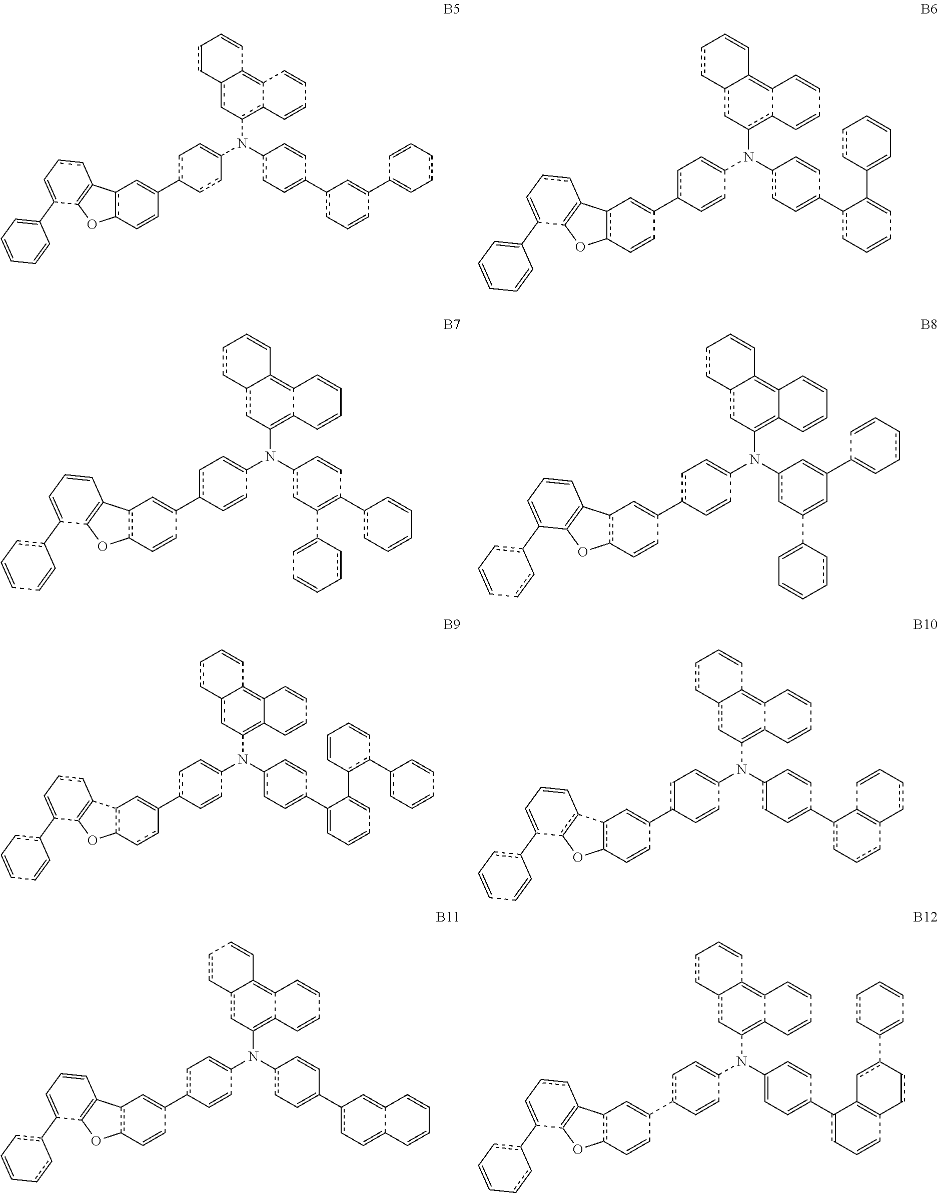 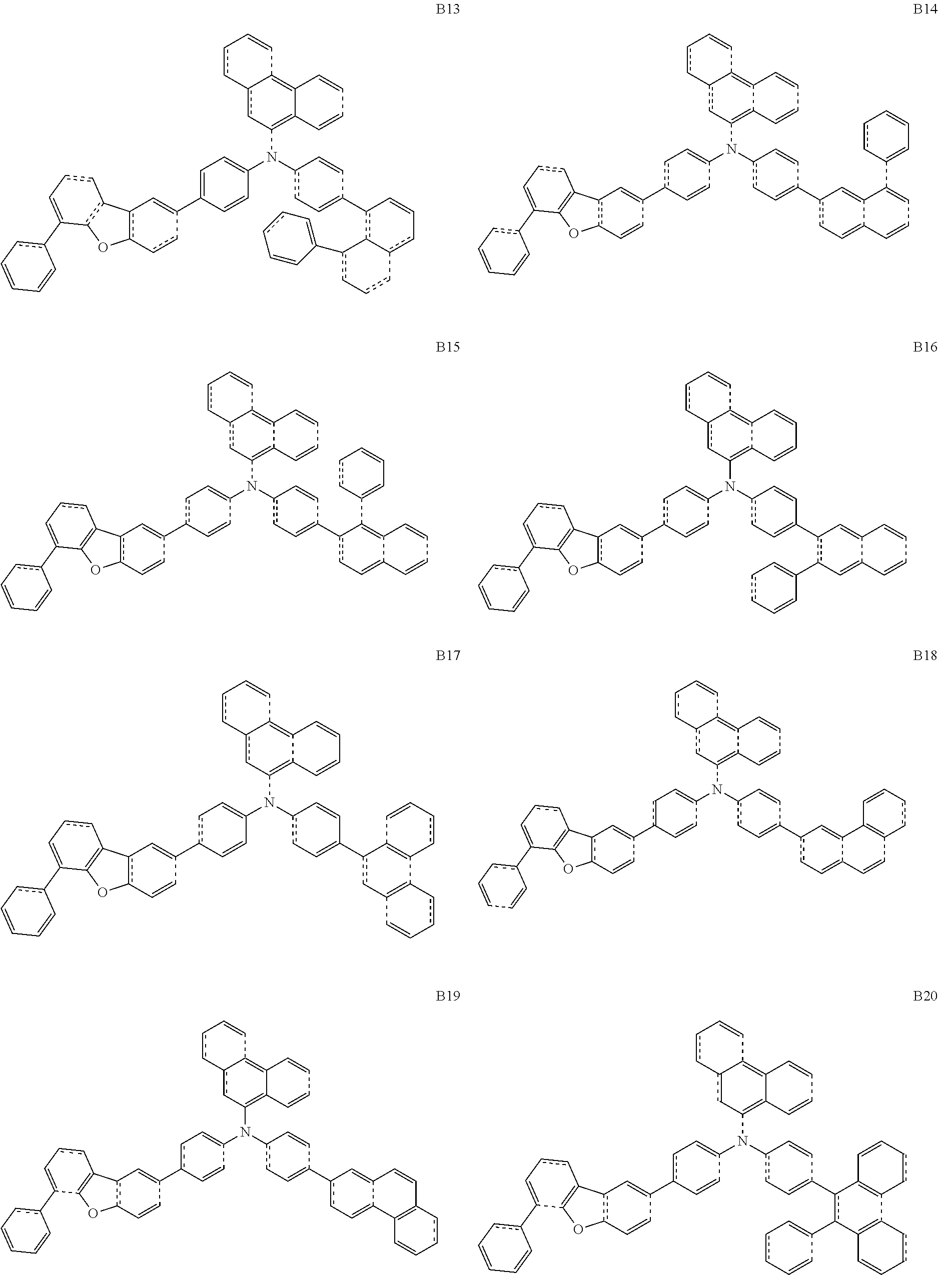 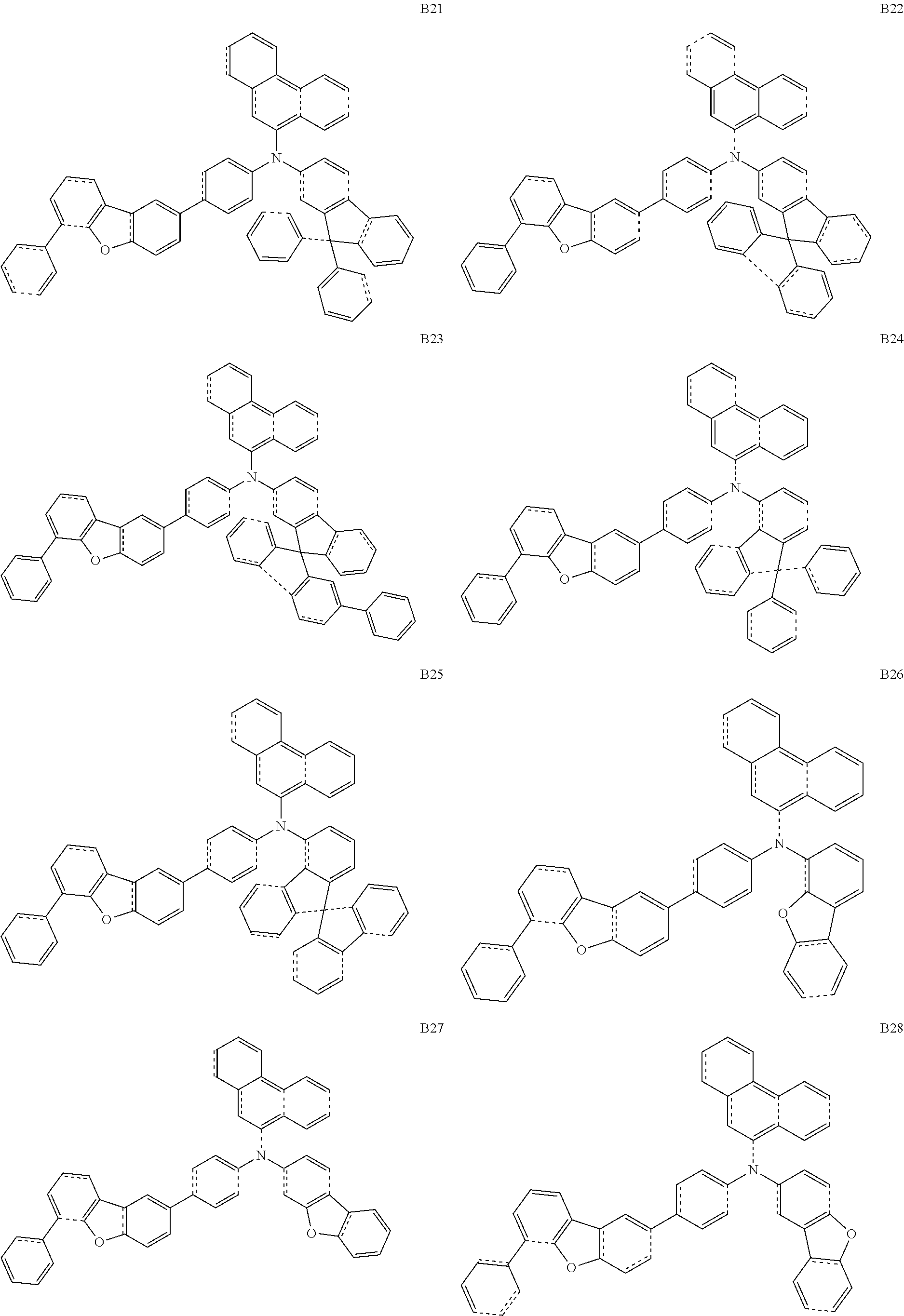 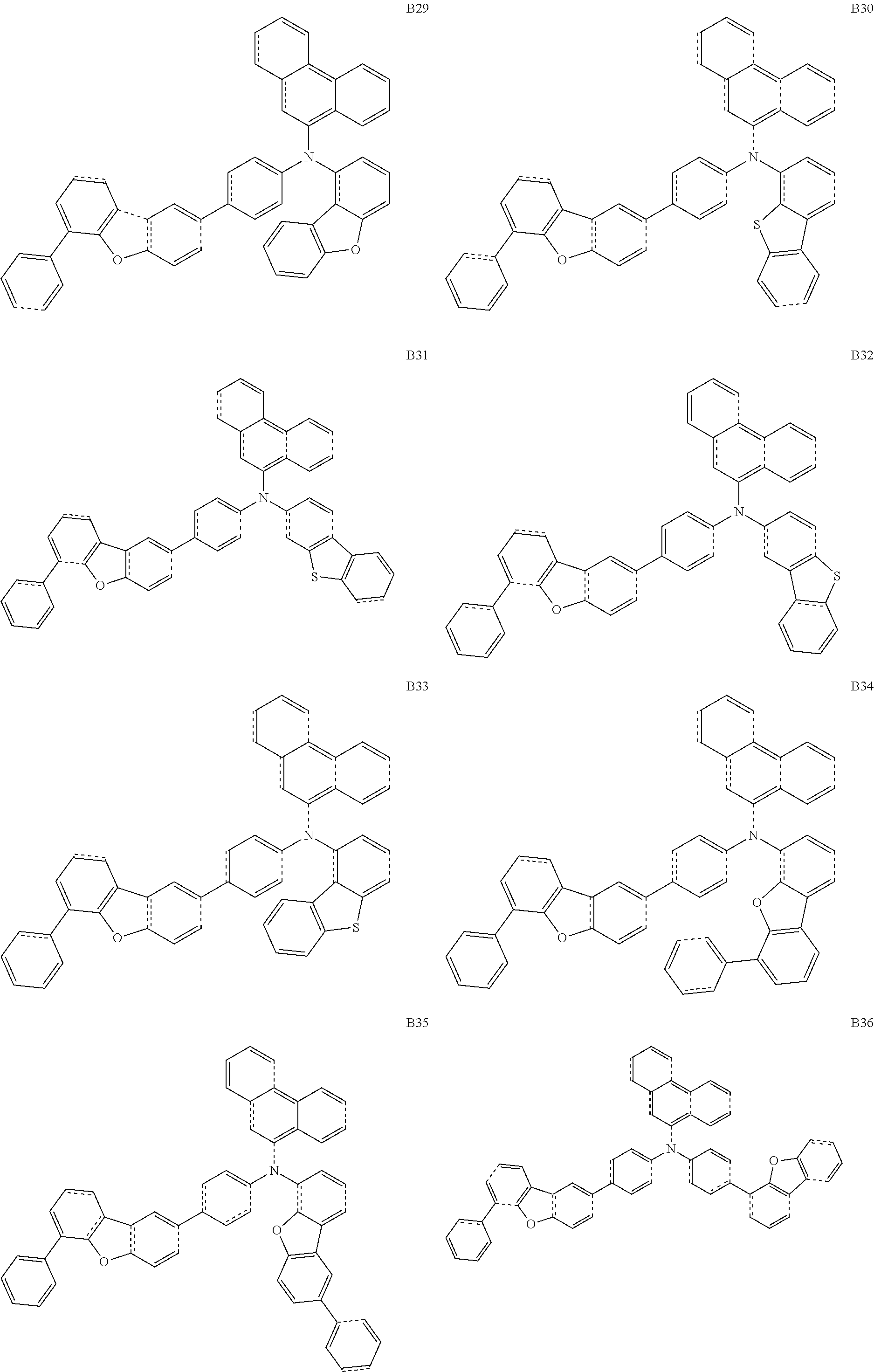 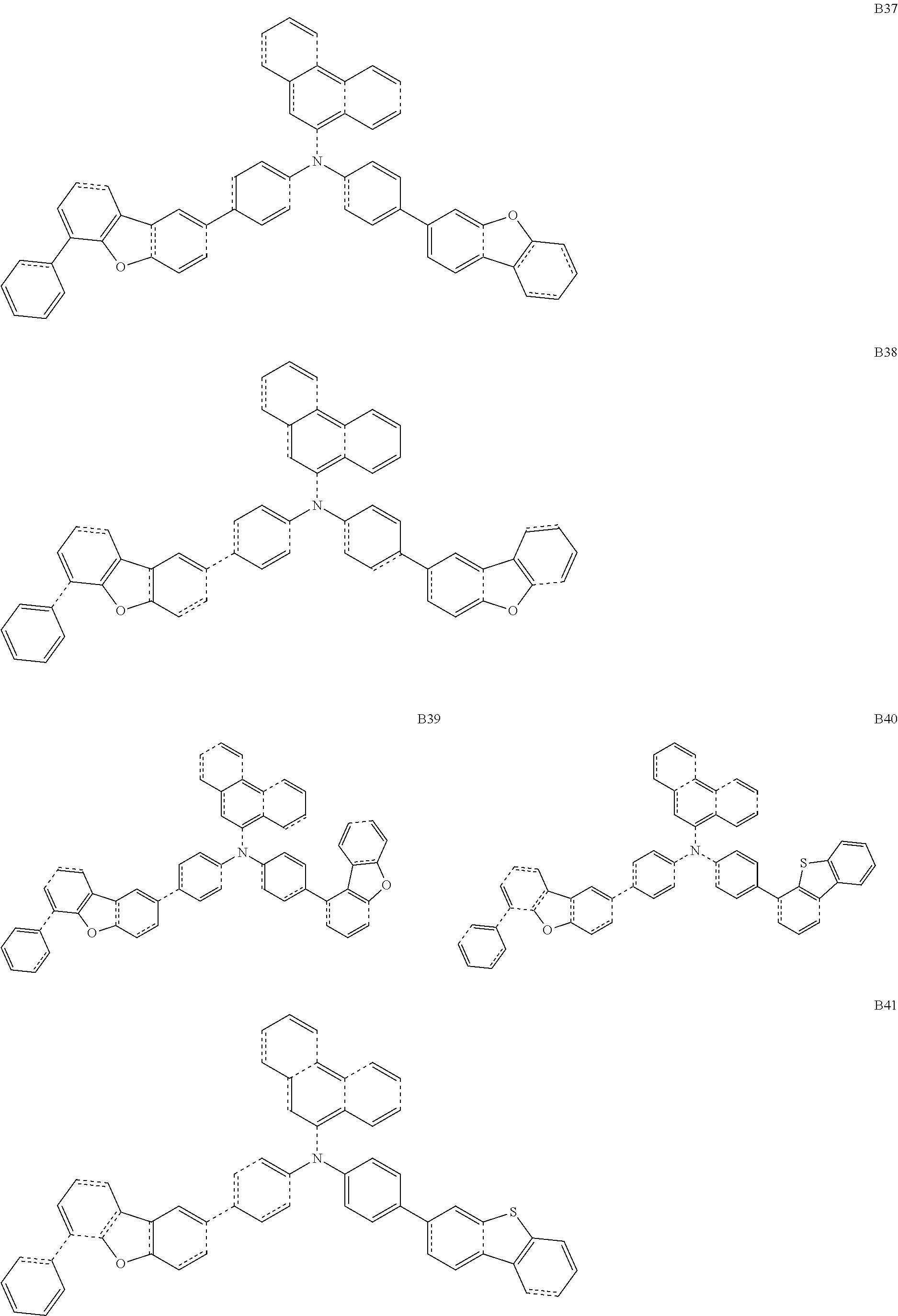 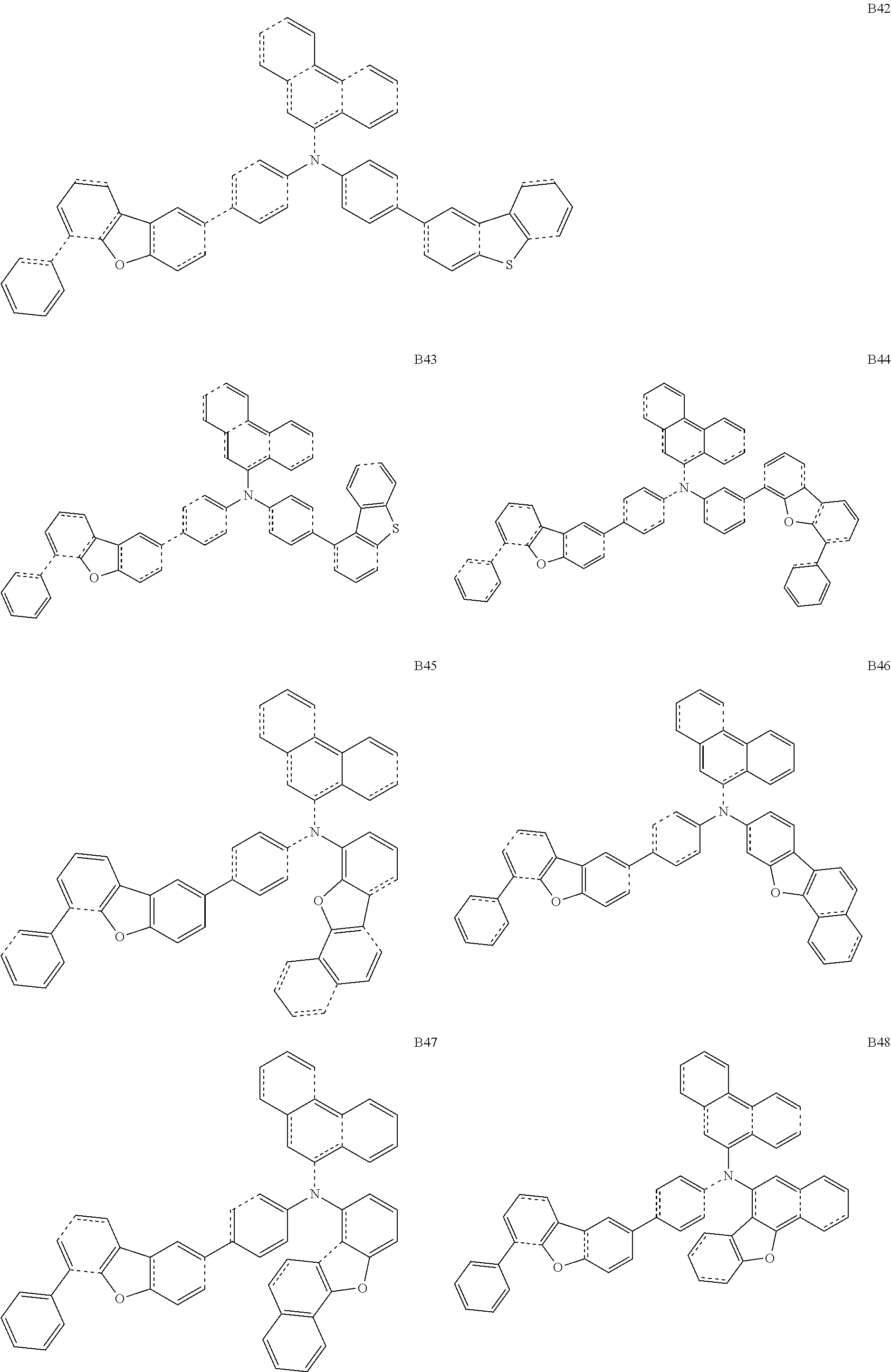 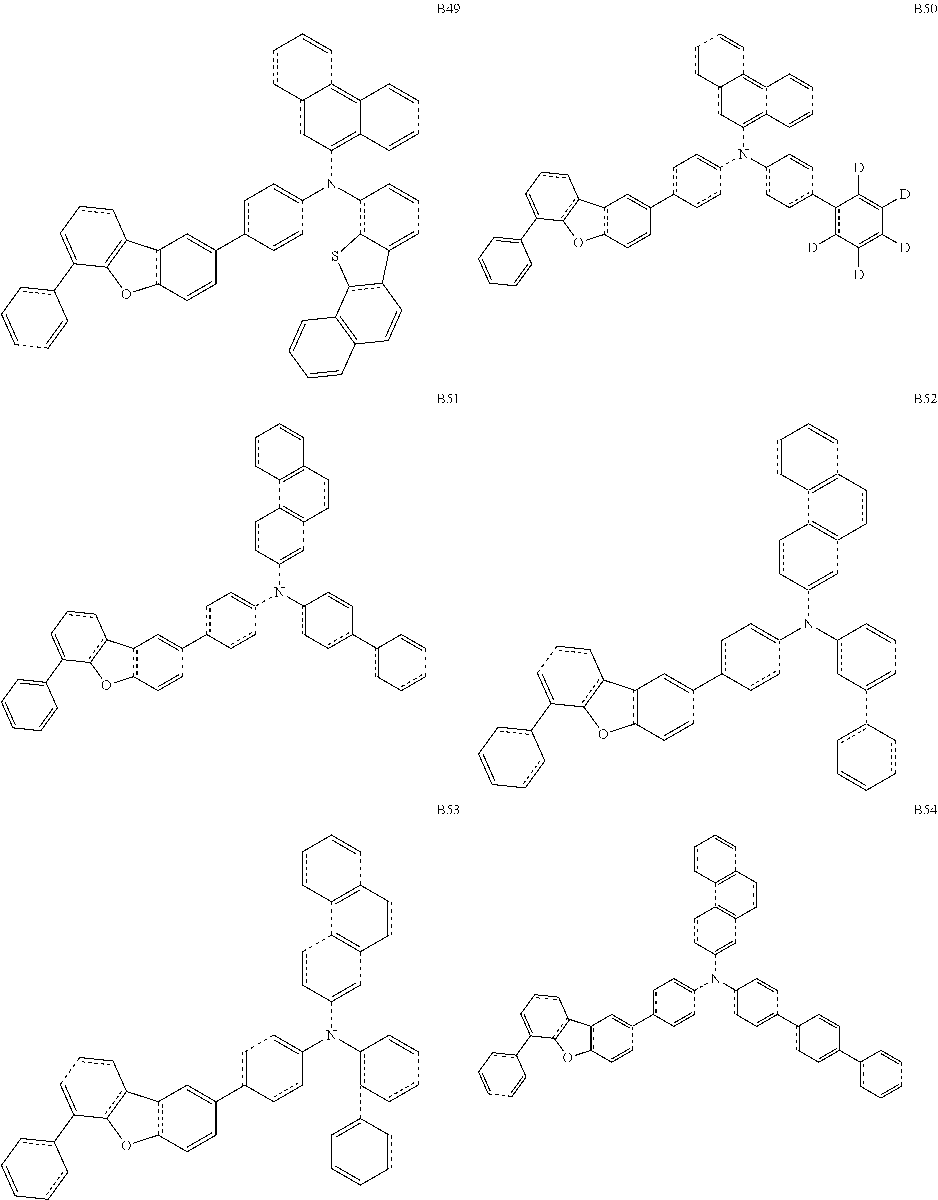 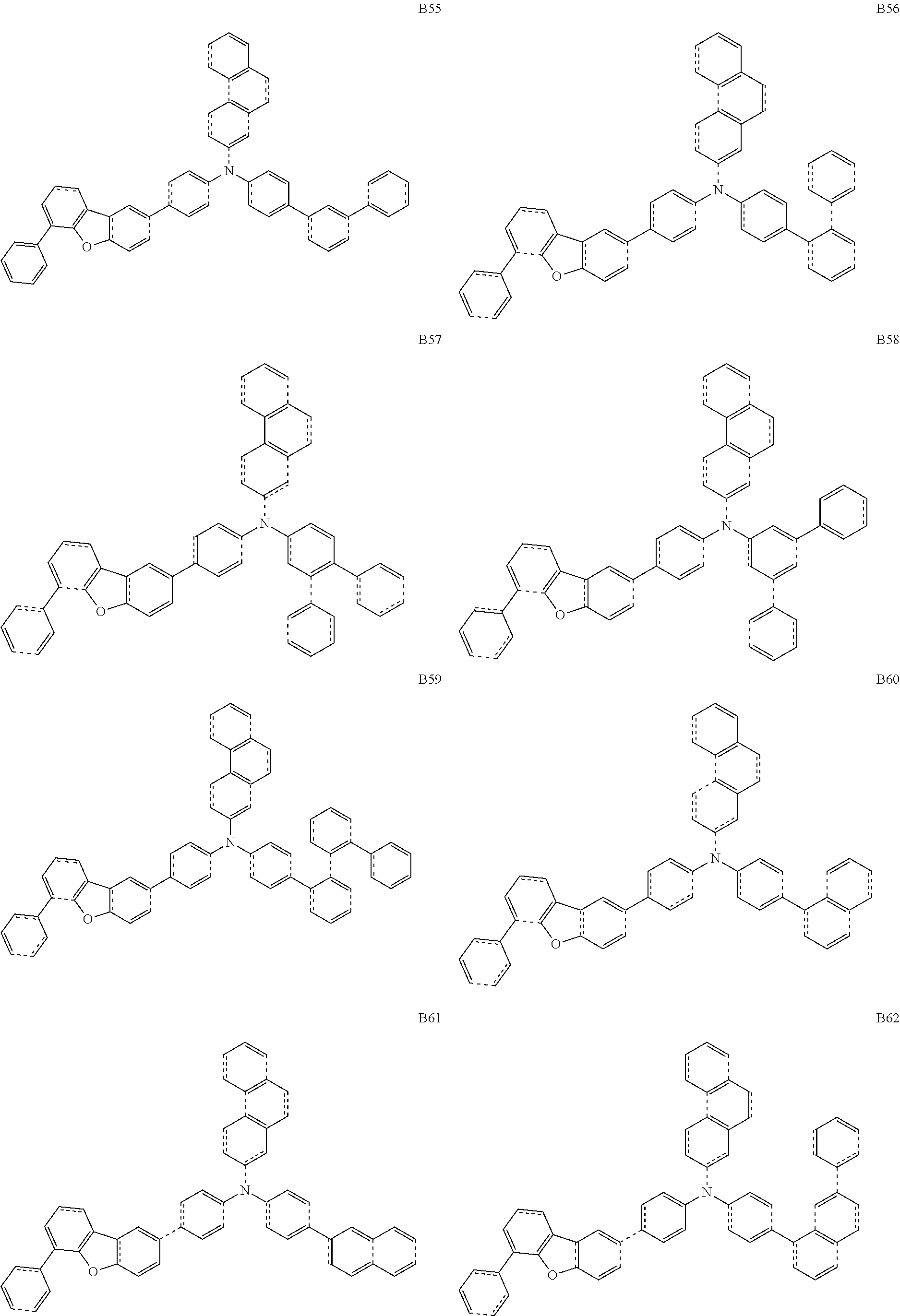 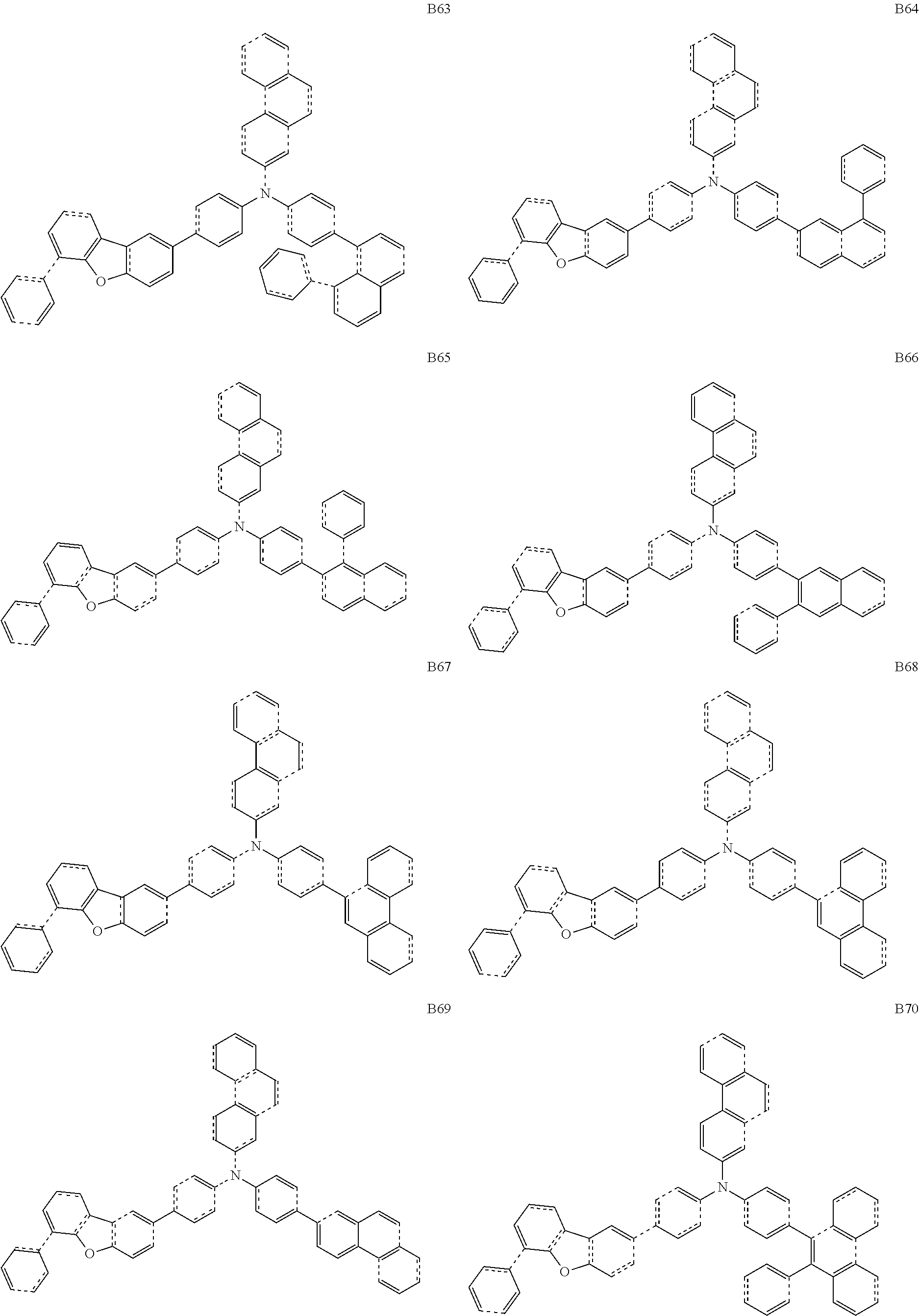 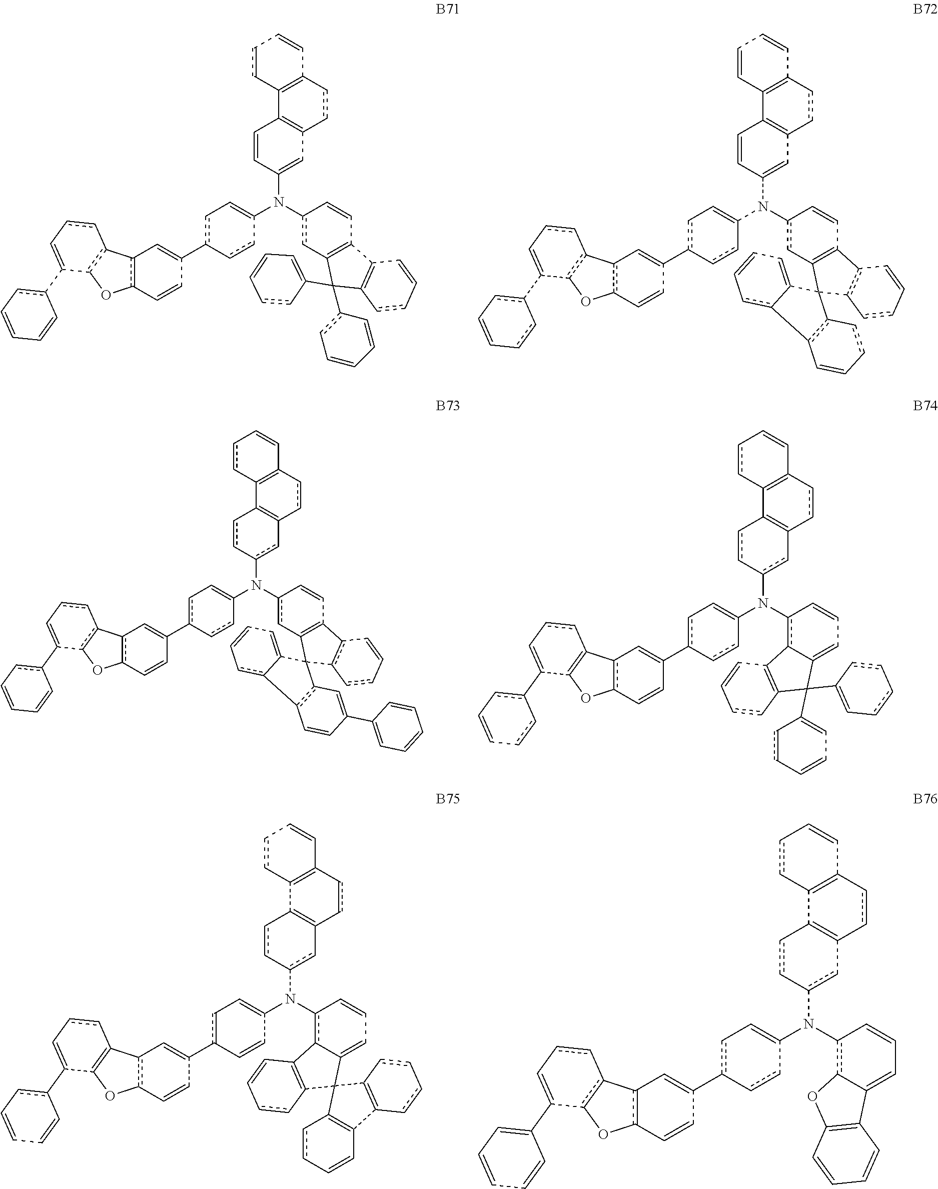 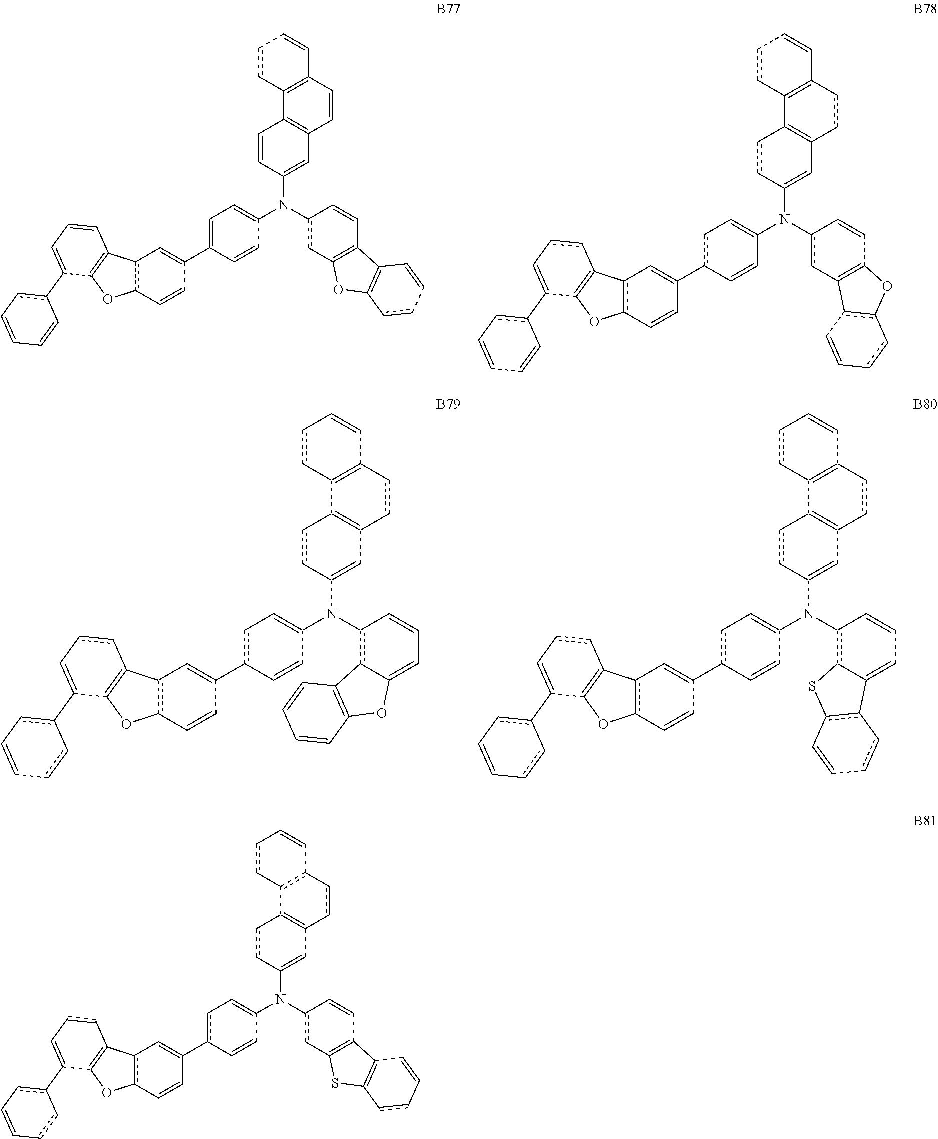 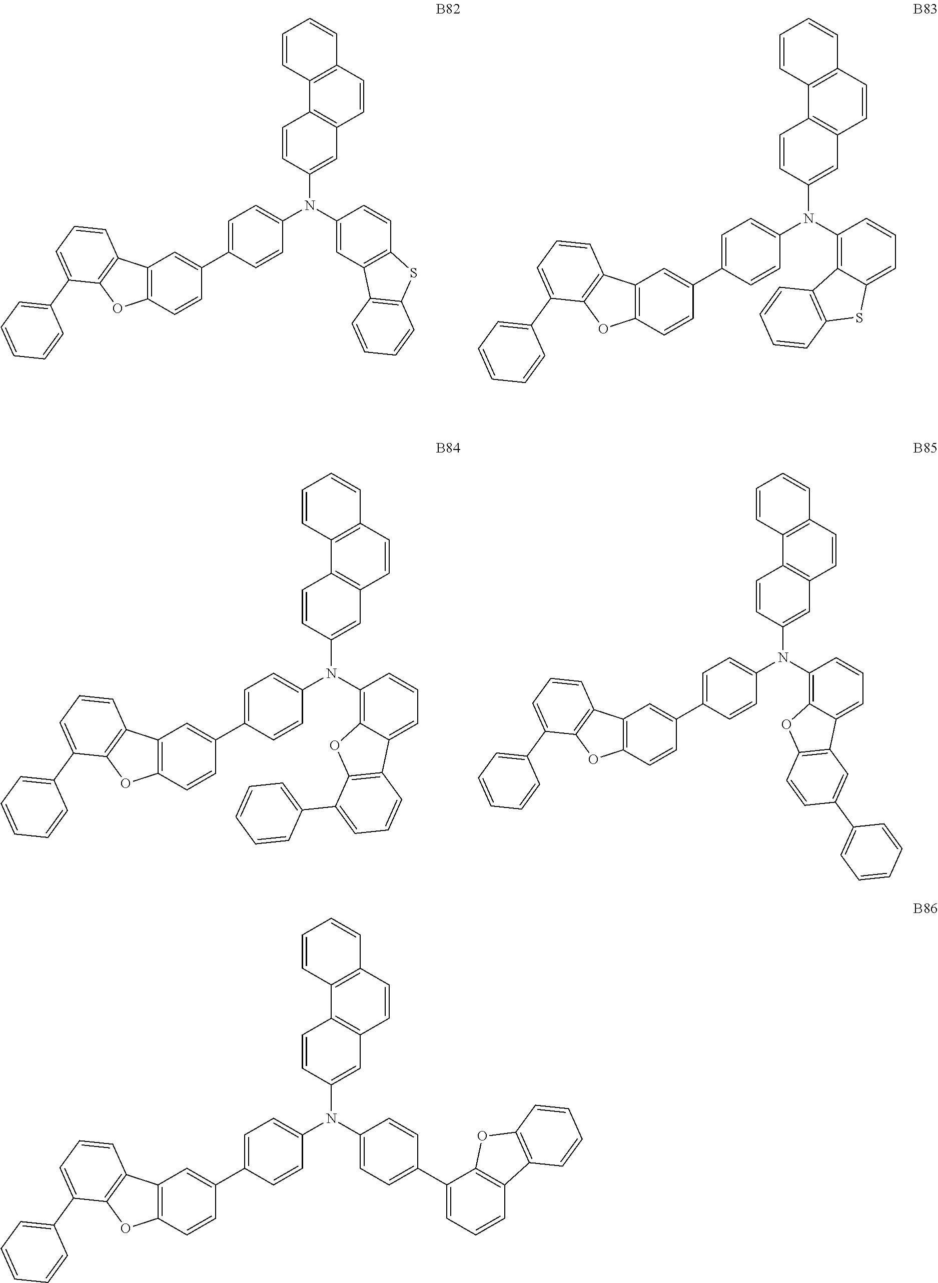 [SMILES: c1ccc(-c2cccc3c2oc2ccc(-c4ccc(N(c5ccc6c(ccc7ccccc76)c5)c5ccc6sc7ccccc7c6c5)cc4)cc23)cc1 ; c1ccc(-c2cccc3c2oc2ccc(-c4ccc(N(c5ccc6c(ccc7ccccc76)c5)c5cccc6sc7ccccc7c56)cc4)cc23)cc1 ; c1ccc(-c2cccc3c2oc2ccc(-c4ccc(N(c5ccc6c(ccc7ccccc76)c5)c5cccc6c5oc5c(-c7ccccc7)cccc56)cc4)cc23)cc1 ; c1ccc(-c2ccc3oc4c(N(c5ccc(-c6ccc7oc8c(-c9ccccc9)cccc8c7c6)cc5)c5ccc6c(ccc7ccccc76)c5)cccc4c3c2)cc1 ; c1ccc(-c2cccc3c2oc2ccc(-c4ccc(N(c5ccc(-c6cccc7c6oc6ccccc67)cc5)c5ccc6c(ccc7ccccc76)c5)cc4)cc23)cc1] 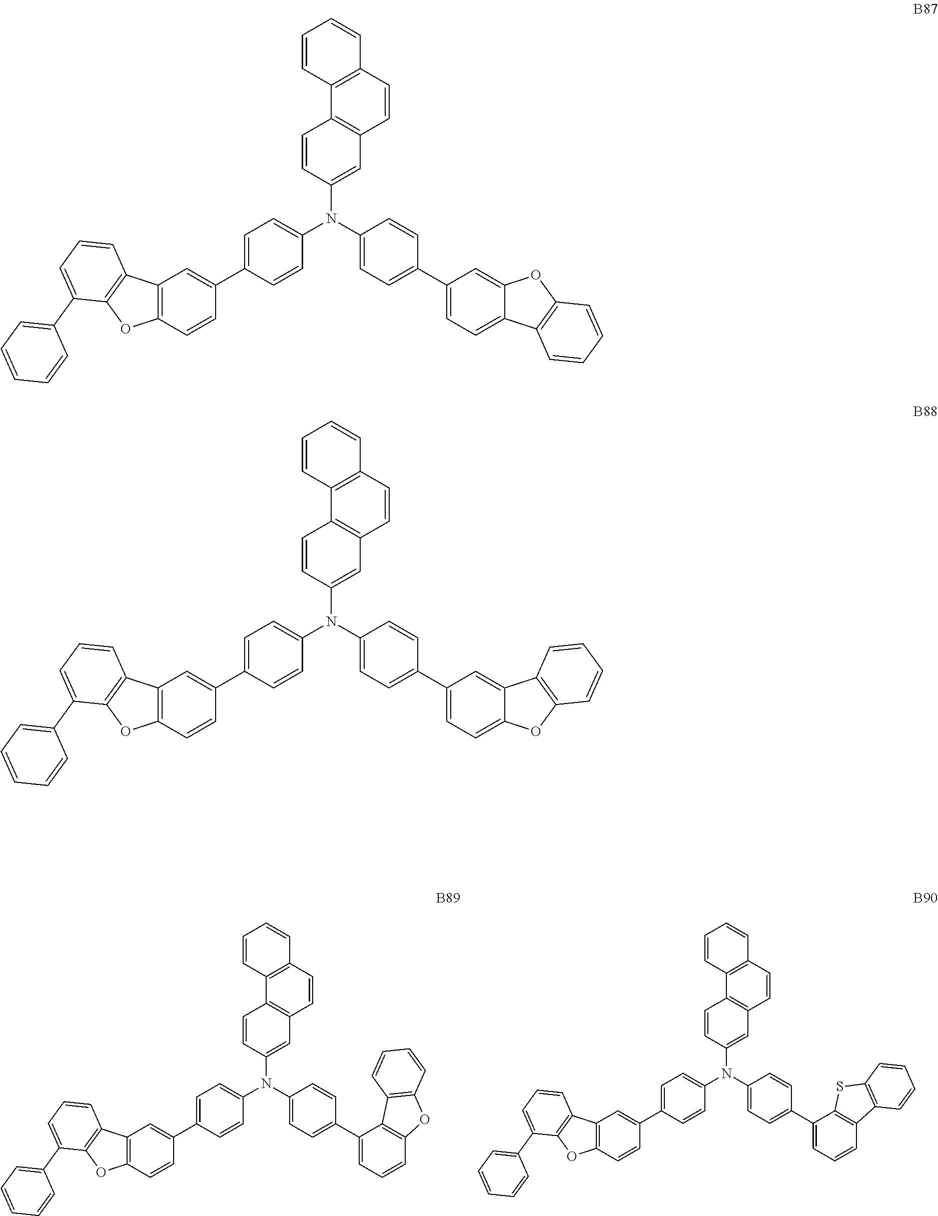 [SMILES: c1ccc(-c2cccc3c2oc2ccc(-c4ccc(N(c5ccc(-c6ccc7c(c6)oc6ccccc67)cc5)c5ccc6c(ccc7ccccc76)c5)cc4)cc23)cc1 ; c1ccc(-c2cccc3c2oc2ccc(-c4ccc(N(c5ccc(-c6ccc7oc8ccccc8c7c6)cc5)c5ccc6c(ccc7ccccc76)c5)cc4)cc23)cc1 ; c1ccc(-c2cccc3c2oc2ccc(-c4ccc(N(c5ccc(-c6cccc7oc8ccccc8c67)cc5)c5ccc6c(ccc7ccccc76)c5)cc4)cc23)cc1 ; c1ccc(-c2cccc3c2oc2ccc(-c4ccc(N(c5ccc(-c6cccc7c6sc6ccccc67)cc5)c5ccc6c(ccc7ccccc76)c5)cc4)cc23)cc1] 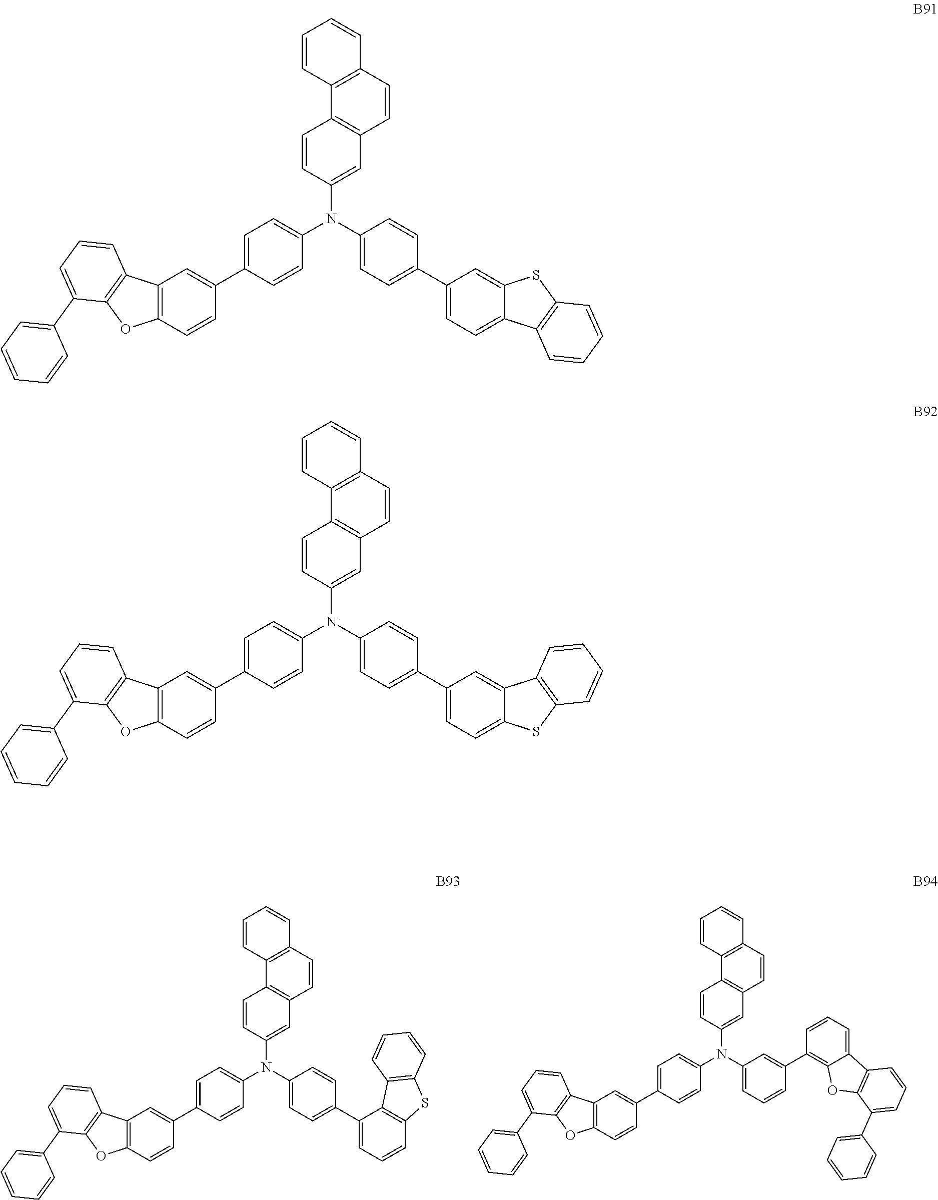 [SMILES: c1ccc(-c2cccc3c2oc2ccc(-c4ccc(N(c5ccc(-c6ccc7c(c6)sc6ccccc67)cc5)c5ccc6c(ccc7ccccc76)c5)cc4)cc23)cc1 ; c1ccc(-c2cccc3c2oc2ccc(-c4ccc(N(c5ccc(-c6ccc7sc8ccccc8c7c6)cc5)c5ccc6c(ccc7ccccc76)c5)cc4)cc23)cc1 ; c1ccc(-c2cccc3c2oc2ccc(-c4ccc(N(c5ccc(-c6cccc7sc8ccccc8c67)cc5)c5ccc6c(ccc7ccccc76)c5)cc4)cc23)cc1 ; c1ccc(-c2cccc3c2oc2ccc(-c4ccc(N(c5cccc(-c6cccc7c6oc6c(-c8ccccc8)cccc67)c5)c5ccc6c(ccc7ccccc76)c5)cc4)cc23)cc1] 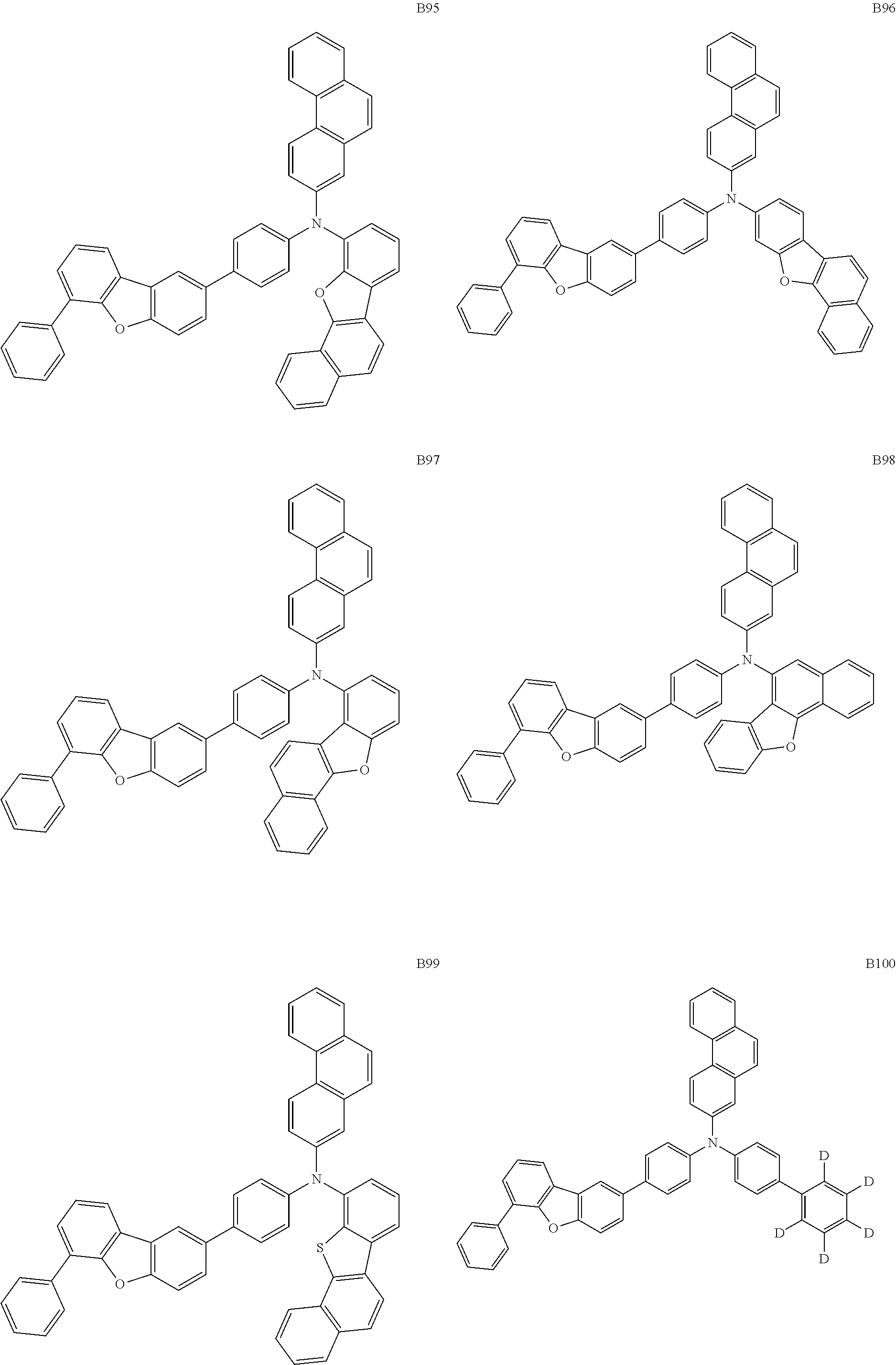 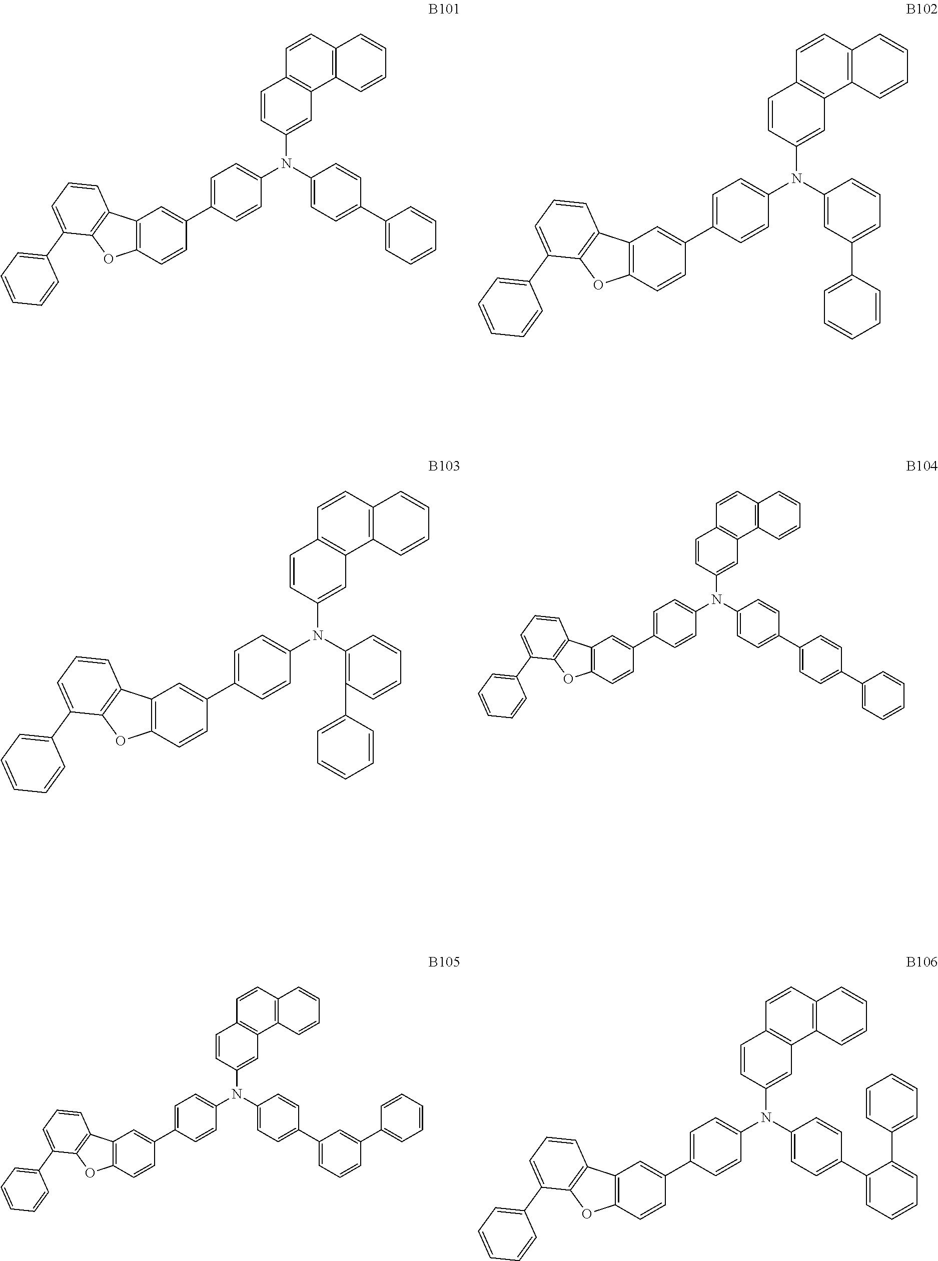 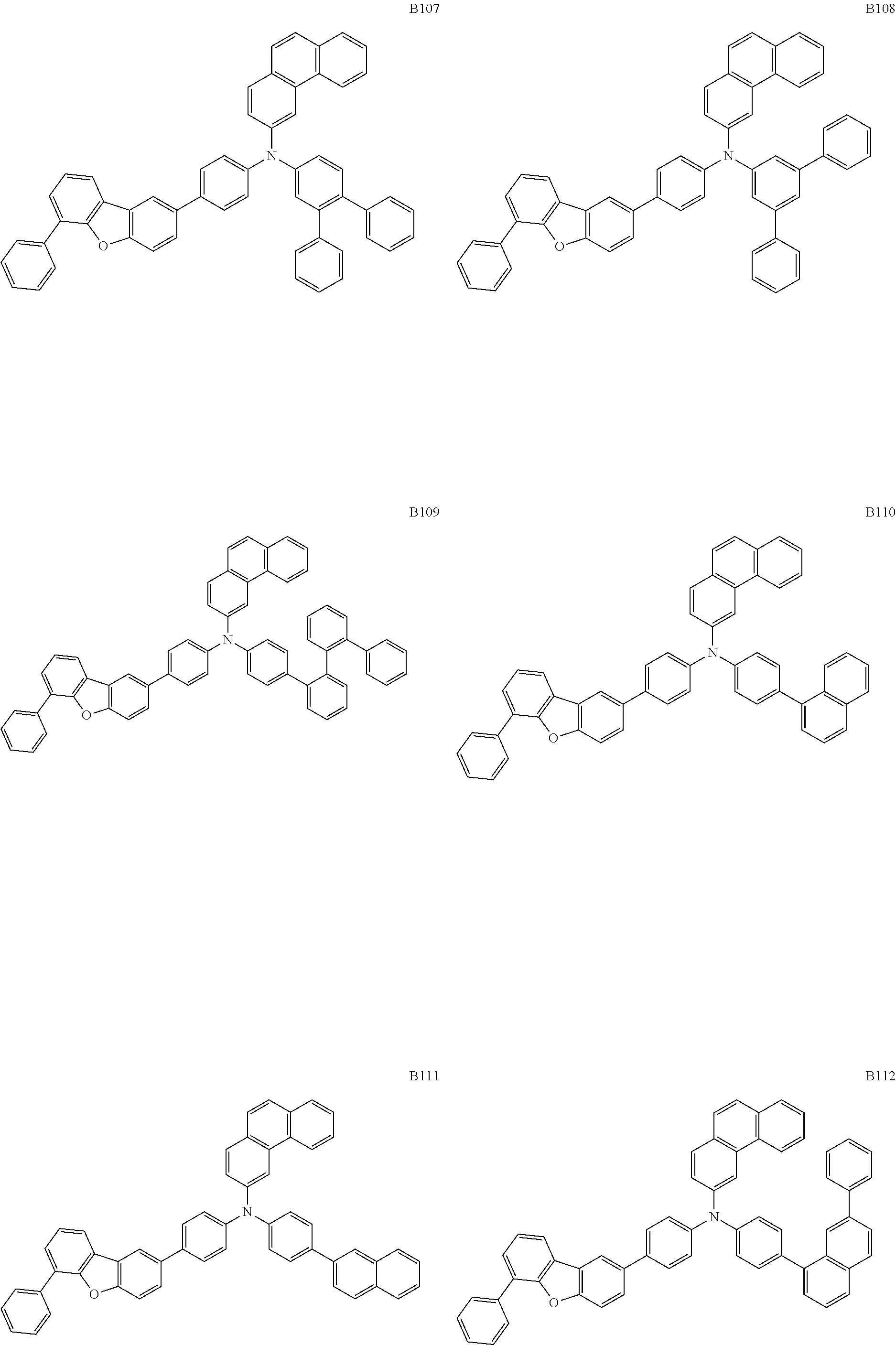 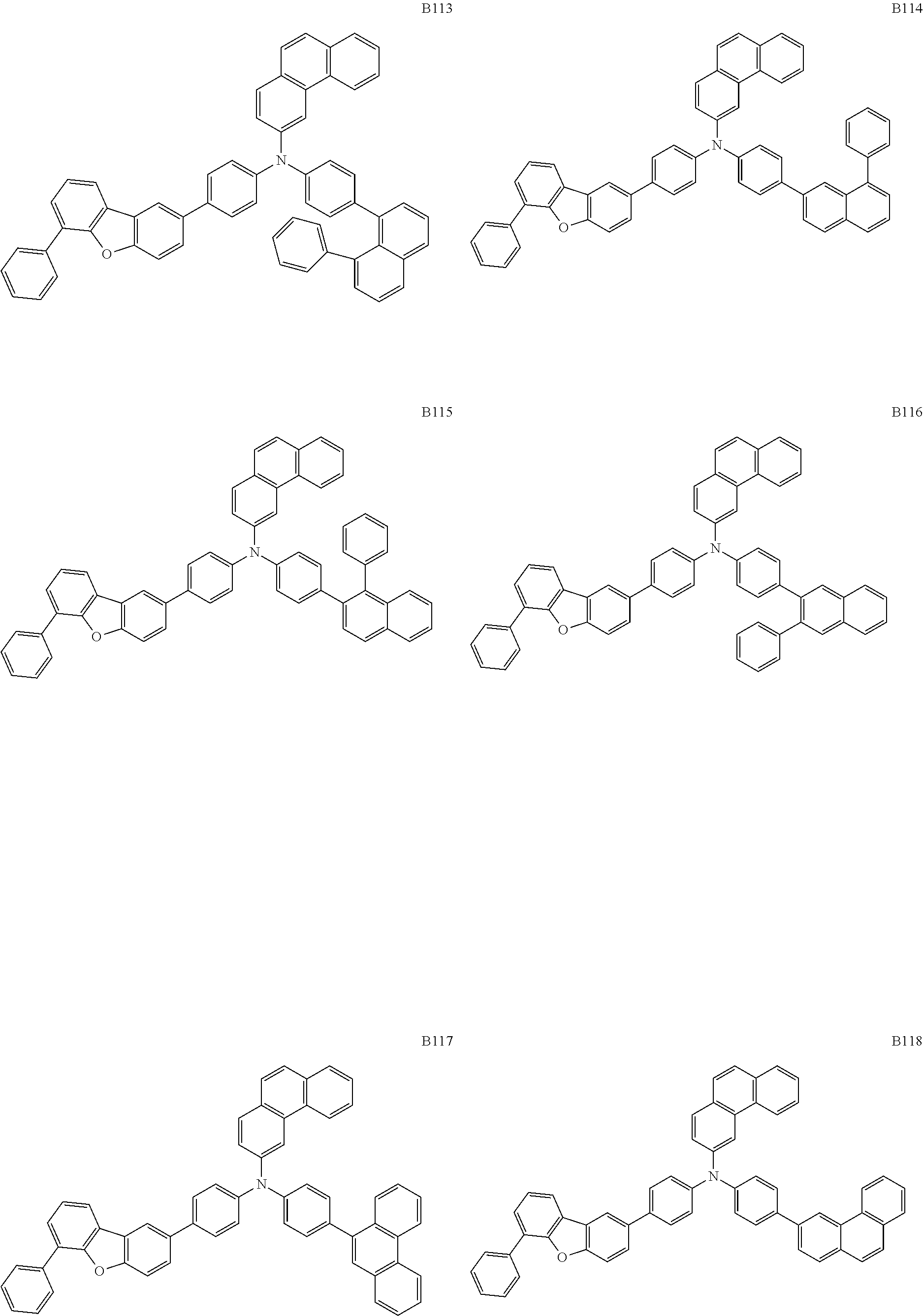 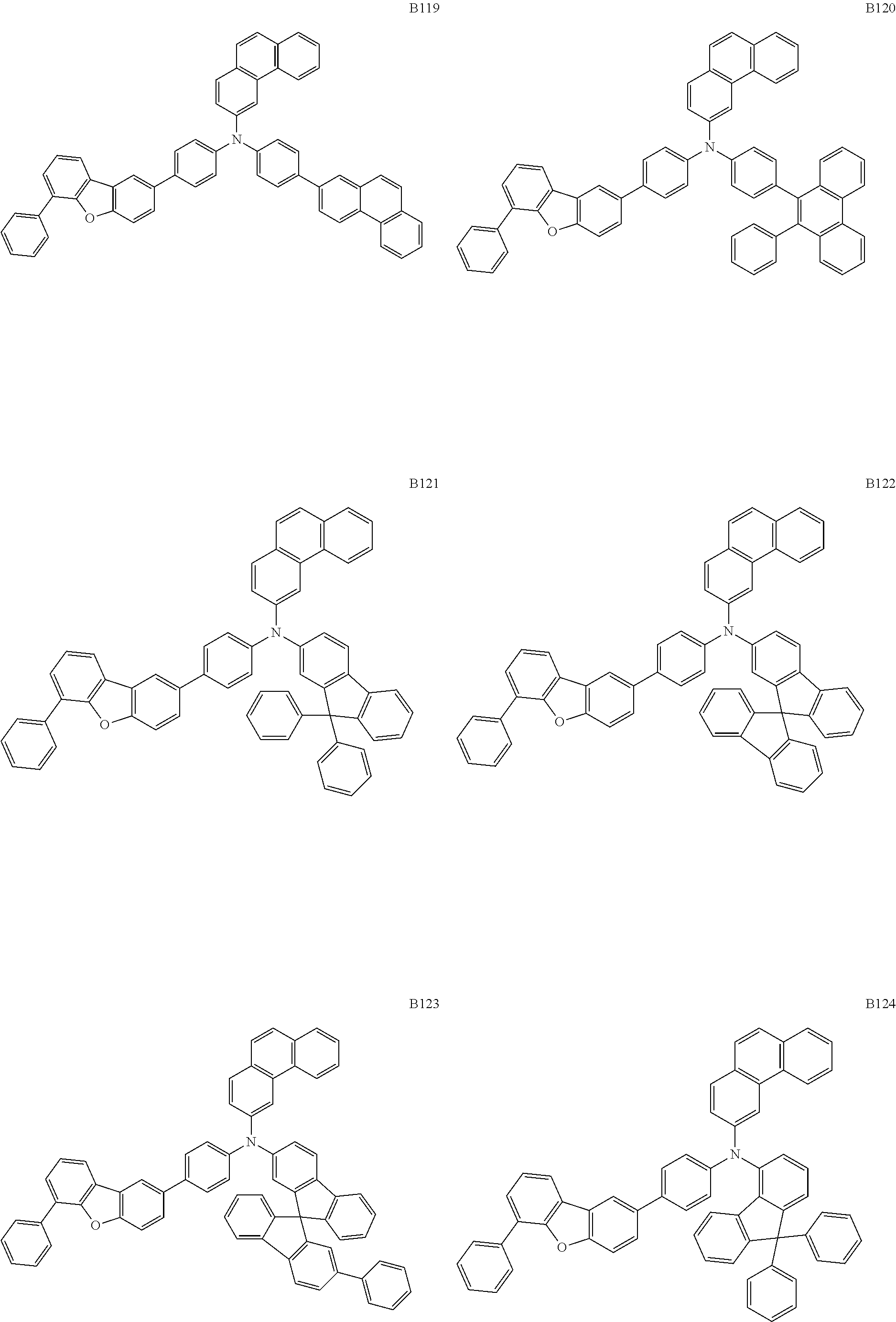 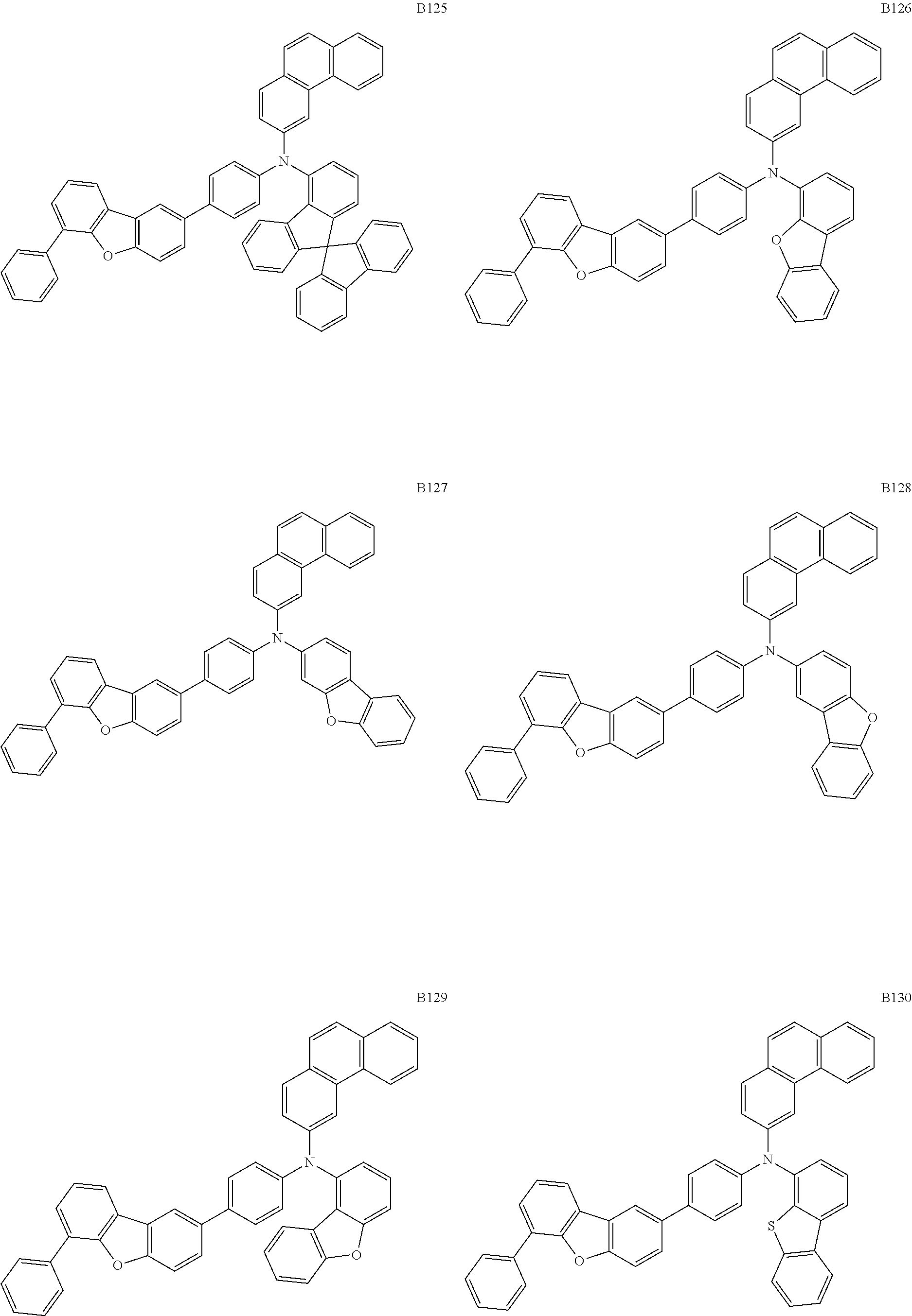 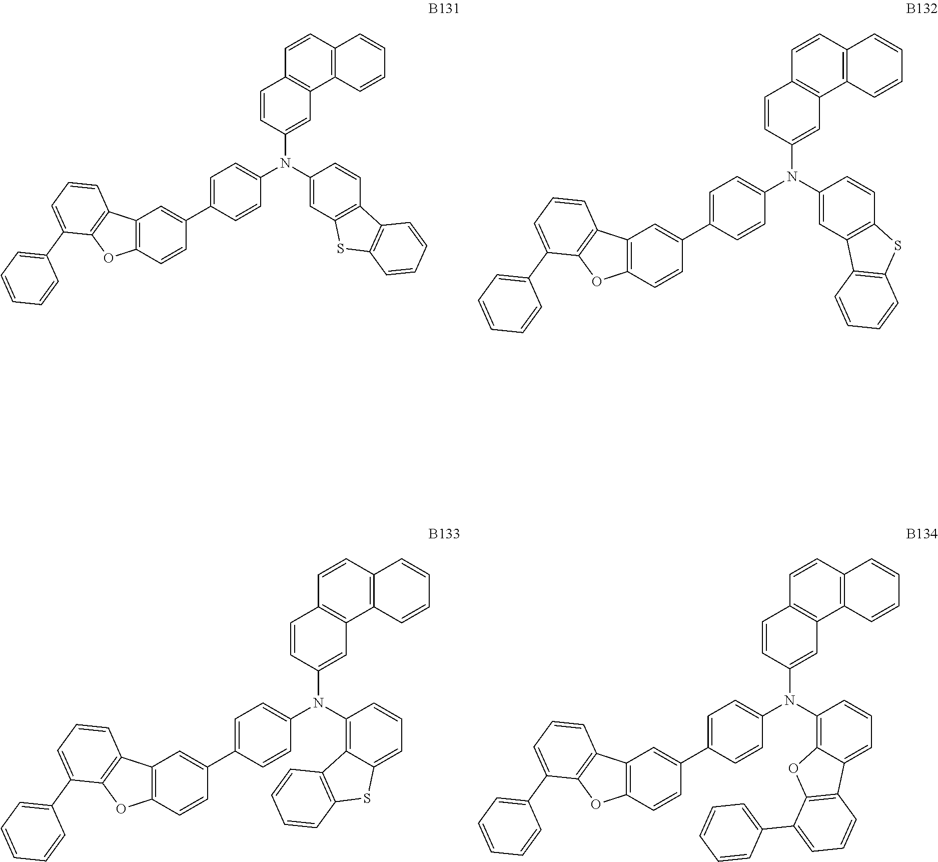 [SMILES: c1ccc(-c2cccc3c2oc2ccc(-c4ccc(N(c5ccc6c(c5)sc5ccccc56)c5ccc6ccc7ccccc7c6c5)cc4)cc23)cc1 ; c1ccc(-c2cccc3c2oc2ccc(-c4ccc(N(c5ccc6ccc7ccccc7c6c5)c5ccc6sc7ccccc7c6c5)cc4)cc23)cc1 ; c1ccc(-c2cccc3c2oc2ccc(-c4ccc(N(c5ccc6ccc7ccccc7c6c5)c5cccc6sc7ccccc7c56)cc4)cc23)cc1 ; c1ccc(-c2cccc3c2oc2ccc(-c4ccc(N(c5ccc6ccc7ccccc7c6c5)c5cccc6c5oc5c(-c7ccccc7)cccc56)cc4)cc23)cc1] 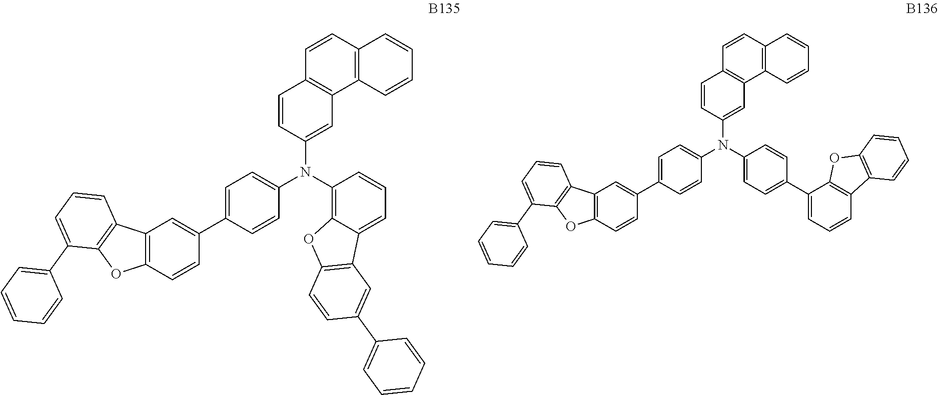 [SMILES: c1ccc(-c2ccc3oc4c(N(c5ccc(-c6ccc7oc8c(-c9ccccc9)cccc8c7c6)cc5)c5ccc6ccc7ccccc7c6c5)cccc4c3c2)cc1 ; c1ccc(-c2cccc3c2oc2ccc(-c4ccc(N(c5ccc(-c6cccc7c6oc6ccccc67)cc5)c5ccc6ccc7ccccc7c6c5)cc4)cc23)cc1] 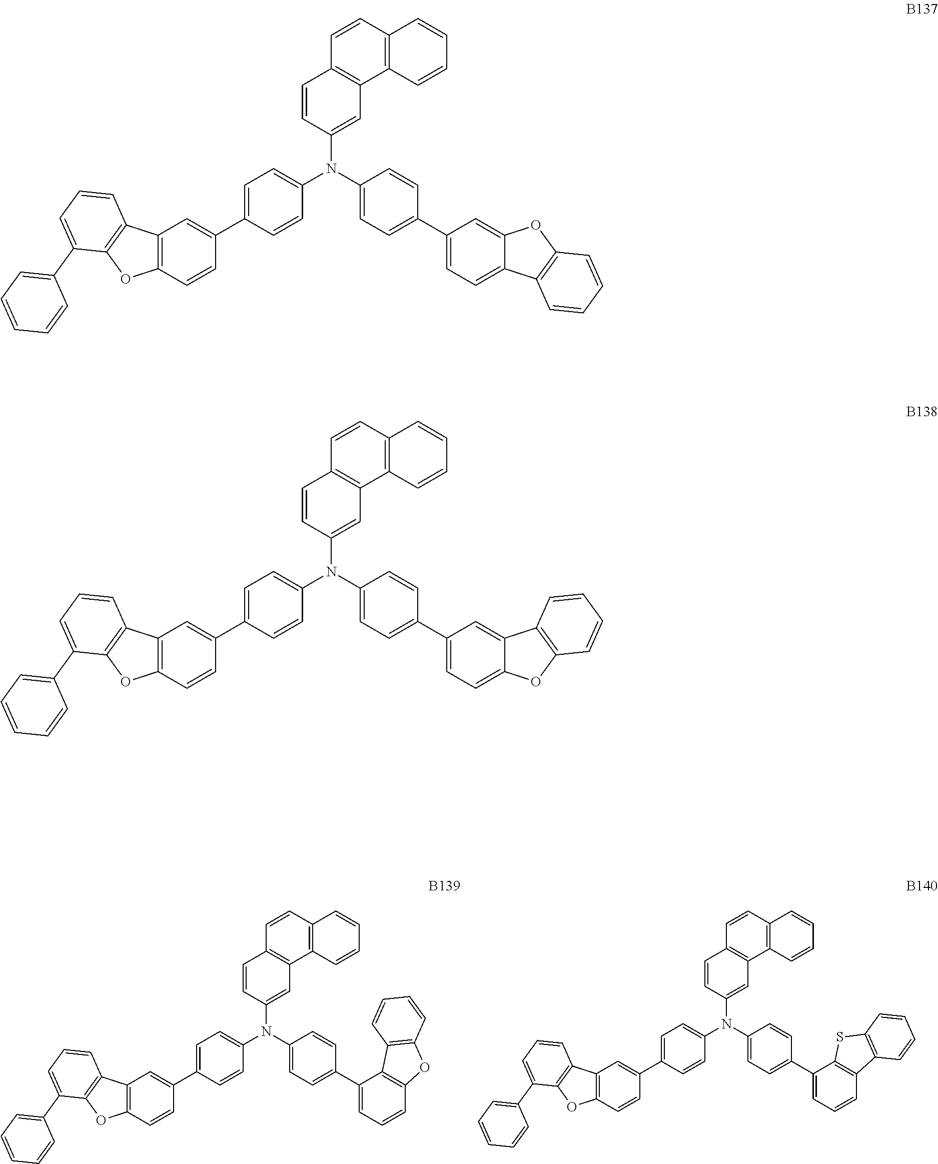 [SMILES: c1ccc(-c2cccc3c2oc2ccc(-c4ccc(N(c5ccc(-c6ccc7c(c6)oc6ccccc67)cc5)c5ccc6ccc7ccccc7c6c5)cc4)cc23)cc1 ; c1ccc(-c2cccc3c2oc2ccc(-c4ccc(N(c5ccc(-c6ccc7oc8ccccc8c7c6)cc5)c5ccc6ccc7ccccc7c6c5)cc4)cc23)cc1 ; c1ccc(-c2cccc3c2oc2ccc(-c4ccc(N(c5ccc(-c6cccc7oc8ccccc8c67)cc5)c5ccc6ccc7ccccc7c6c5)cc4)cc23)cc1 ; c1ccc(-c2cccc3c2oc2ccc(-c4ccc(N(c5ccc(-c6cccc7c6sc6ccccc67)cc5)c5ccc6ccc7ccccc7c6c5)cc4)cc23)cc1] 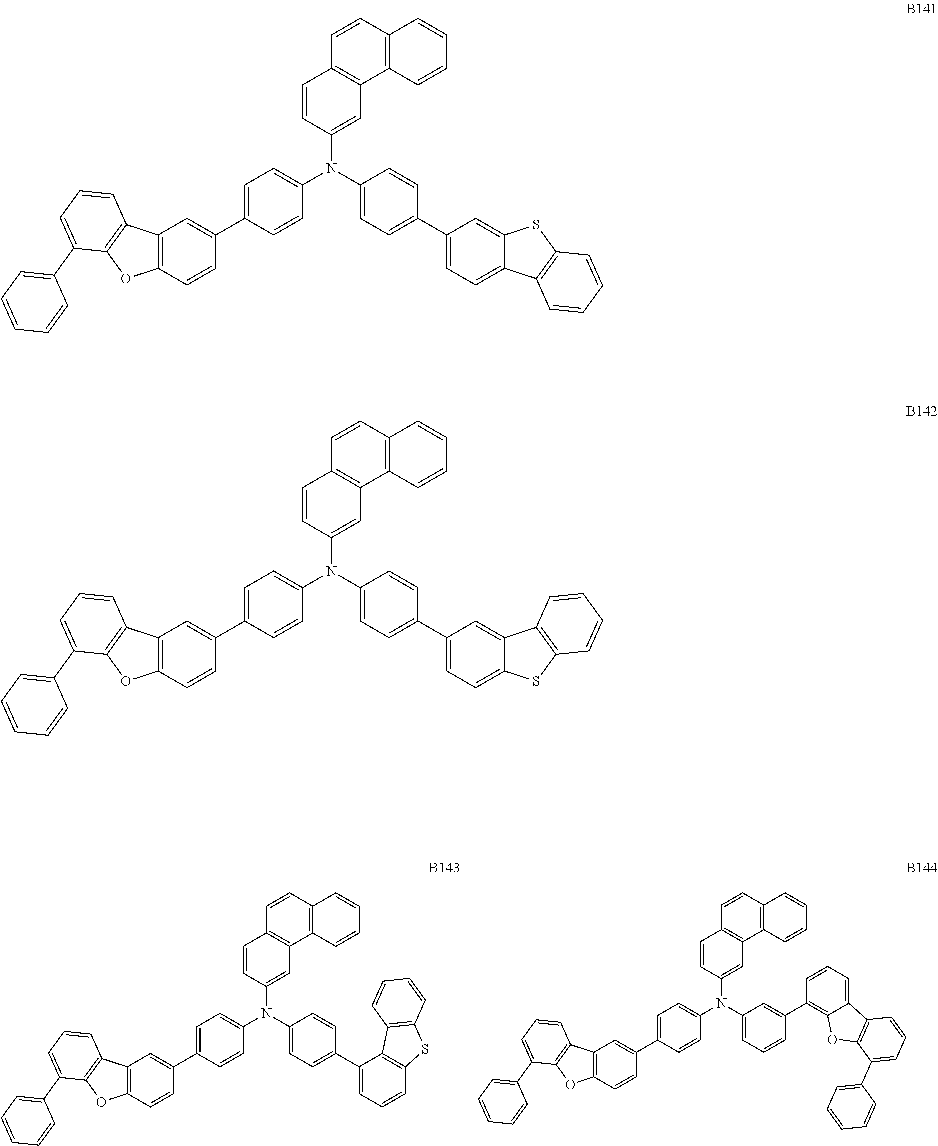 [SMILES: c1ccc(-c2cccc3c2oc2ccc(-c4ccc(N(c5ccc(-c6ccc7c(c6)sc6ccccc67)cc5)c5ccc6ccc7ccccc7c6c5)cc4)cc23)cc1 ; c1ccc(-c2cccc3c2oc2ccc(-c4ccc(N(c5ccc(-c6ccc7sc8ccccc8c7c6)cc5)c5ccc6ccc7ccccc7c6c5)cc4)cc23)cc1 ; c1ccc(-c2cccc3c2oc2ccc(-c4ccc(N(c5ccc(-c6cccc7sc8ccccc8c67)cc5)c5ccc6ccc7ccccc7c6c5)cc4)cc23)cc1 ; c1ccc(-c2cccc3c2oc2ccc(-c4ccc(N(c5cccc(-c6cccc7c6oc6c(-c8ccccc8)cccc67)c5)c5ccc6ccc7ccccc7c6c5)cc4)cc23)cc1] 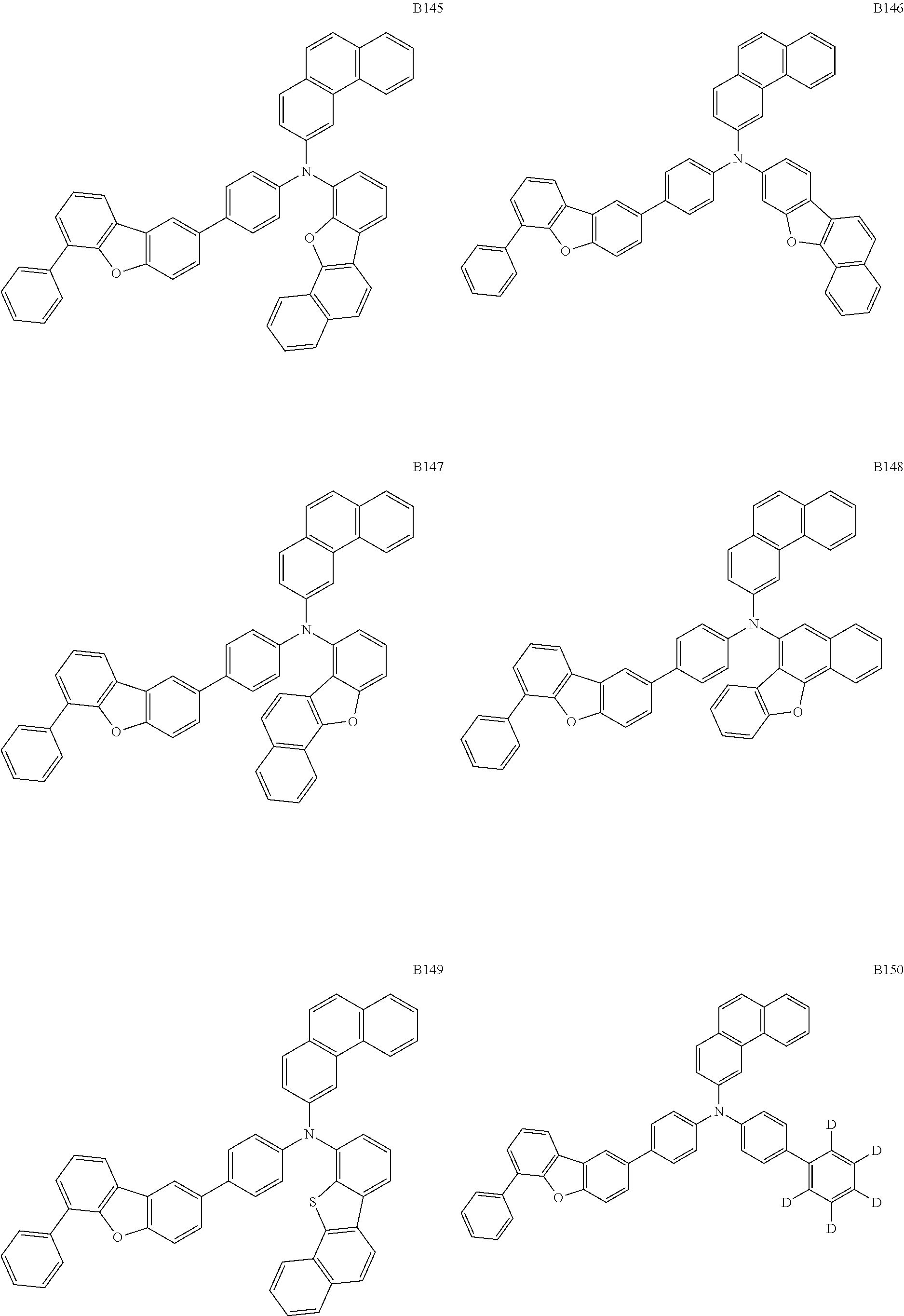 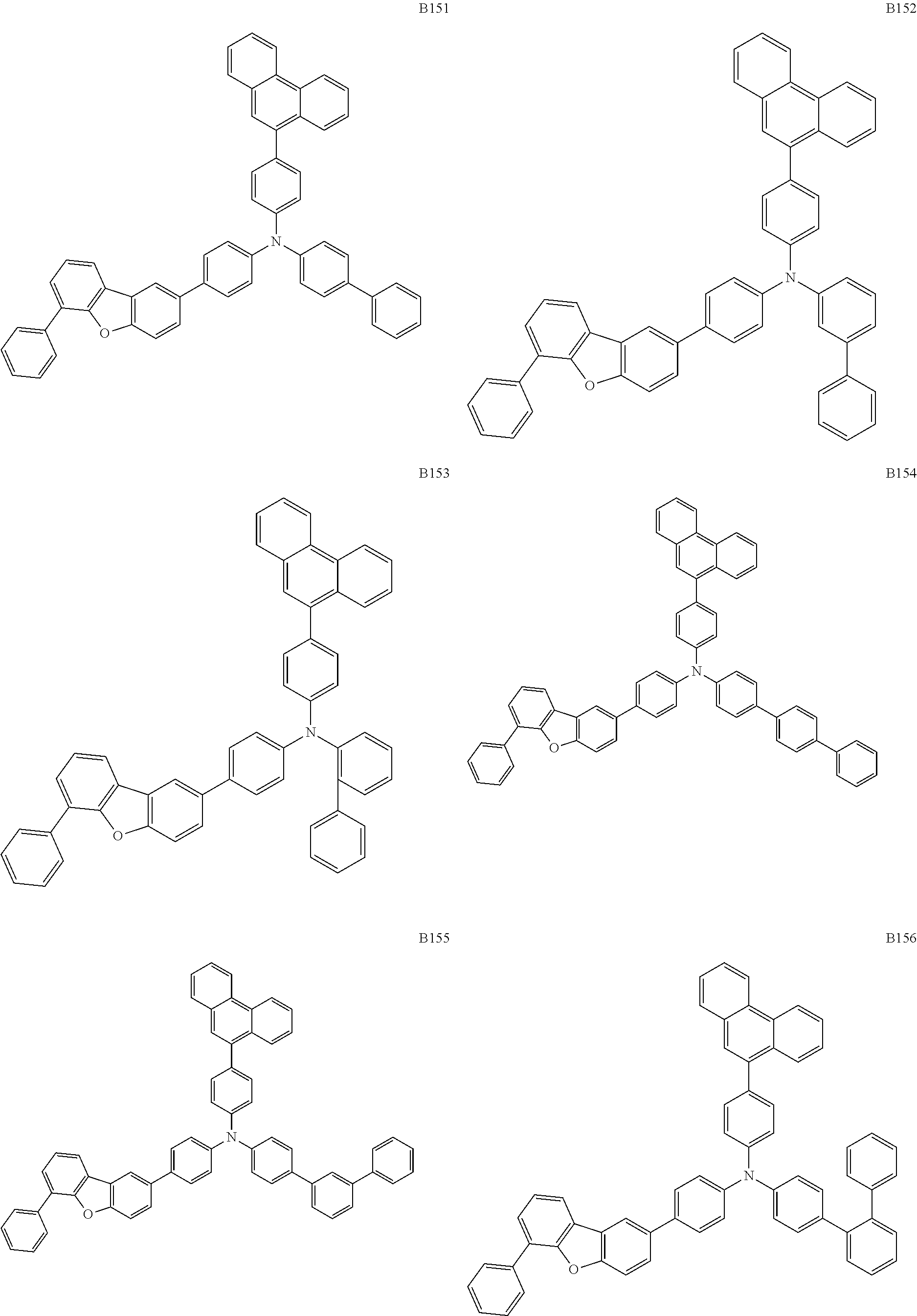 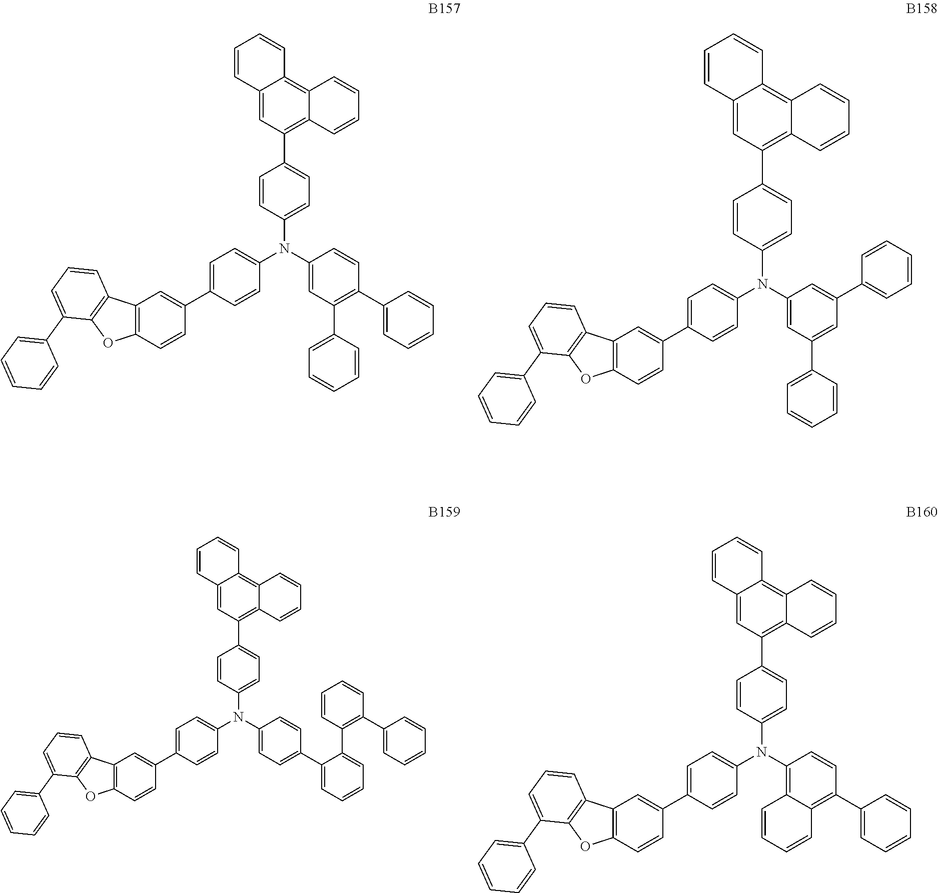 [SMILES: c1ccc(-c2ccc(N(c3ccc(-c4ccc5oc6c(-c7ccccc7)cccc6c5c4)cc3)c3ccc(-c4cc5ccccc5c5ccccc45)cc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc(-c4cc5ccccc5c5ccccc45)cc3)c2)cc1 ; c1ccc(-c2ccccc2-c2ccccc2-c2ccc(N(c3ccc(-c4ccc5oc6c(-c7ccccc7)cccc6c5c4)cc3)c3ccc(-c4cc5ccccc5c5ccccc45)cc3)cc2)cc1 ; c1ccc(-c2ccc(N(c3ccc(-c4ccc5oc6c(-c7ccccc7)cccc6c5c4)cc3)c3ccc(-c4cc5ccccc5c5ccccc45)cc3)c3ccccc23)cc1] 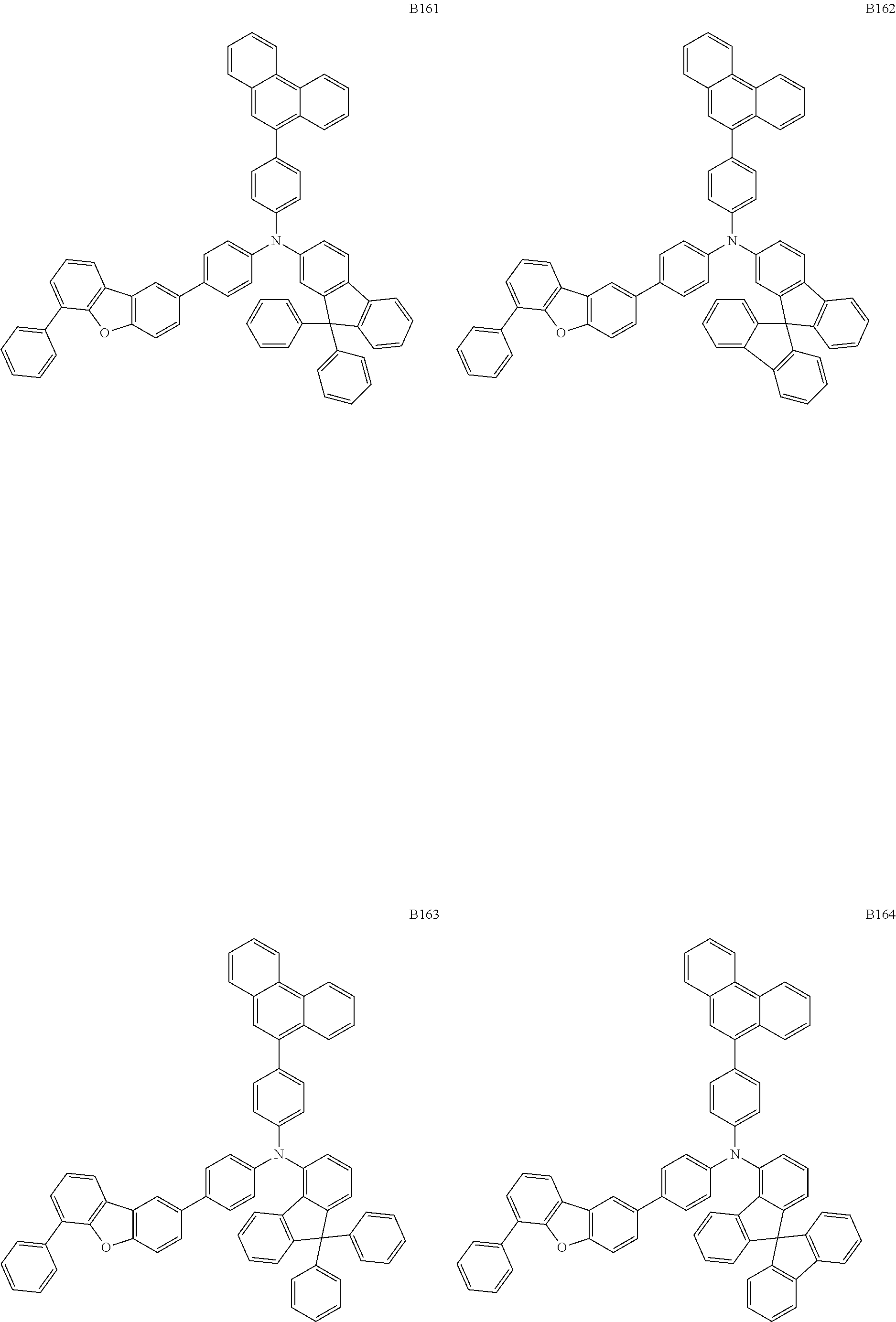 [SMILES: c1ccc(-c2cccc3c2oc2ccc(-c4ccc(N(c5ccc(-c6cc7ccccc7c7ccccc67)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6cc7ccccc7c7ccccc67)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6cc7ccccc7c7ccccc67)cc5)c5cccc6c5-c5ccccc5C6(c5ccccc5)c5ccccc5)cc4)cc23)cc1 ; c1ccc(-c2cccc3c2oc2ccc(-c4ccc(N(c5ccc(-c6cc7ccccc7c7ccccc67)cc5)c5cccc6c5-c5ccccc5C65c6ccccc6-c6ccccc65)cc4)cc23)cc1] 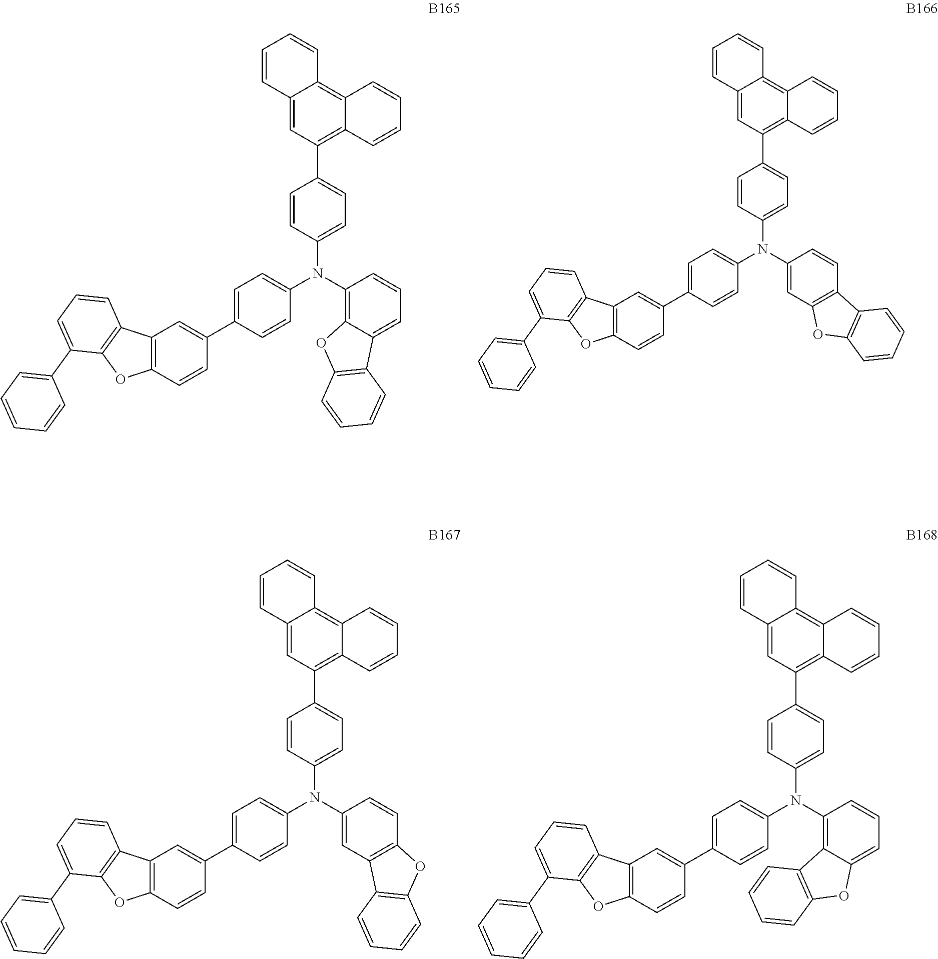 [SMILES: c1ccc(-c2cccc3c2oc2ccc(-c4ccc(N(c5ccc(-c6cc7ccccc7c7ccccc67)cc5)c5cccc6c5oc5ccccc56)cc4)cc23)cc1 ; c1ccc(-c2cccc3c2oc2ccc(-c4ccc(N(c5ccc(-c6cc7ccccc7c7ccccc67)cc5)c5ccc6c(c5)oc5ccccc56)cc4)cc23)cc1 ; c1ccc(-c2cccc3c2oc2ccc(-c4ccc(N(c5ccc(-c6cc7ccccc7c7ccccc67)cc5)c5ccc6oc7ccccc7c6c5)cc4)cc23)cc1 ; c1ccc(-c2cccc3c2oc2ccc(-c4ccc(N(c5ccc(-c6cc7ccccc7c7ccccc67)cc5)c5cccc6oc7ccccc7c56)cc4)cc23)cc1] 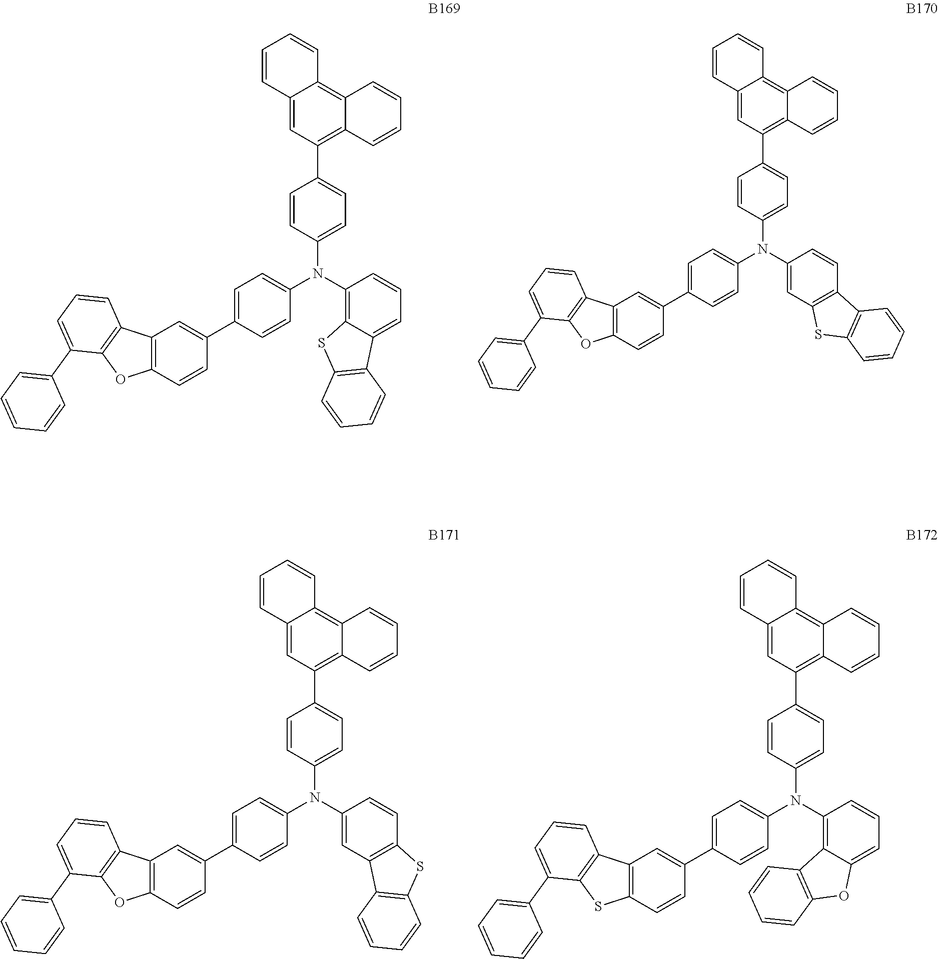 [SMILES: c1ccc(-c2cccc3c2oc2ccc(-c4ccc(N(c5ccc(-c6cc7ccccc7c7ccccc67)cc5)c5cccc6c5sc5ccccc56)cc4)cc23)cc1 ; c1ccc(-c2cccc3c2oc2ccc(-c4ccc(N(c5ccc(-c6cc7ccccc7c7ccccc67)cc5)c5ccc6c(c5)sc5ccccc56)cc4)cc23)cc1 ; c1ccc(-c2cccc3c2oc2ccc(-c4ccc(N(c5ccc(-c6cc7ccccc7c7ccccc67)cc5)c5ccc6sc7ccccc7c6c5)cc4)cc23)cc1 ; c1ccc(-c2cccc3c2sc2ccc(-c4ccc(N(c5ccc(-c6cc7ccccc7c7ccccc67)cc5)c5cccc6oc7ccccc7c56)cc4)cc23)cc1] 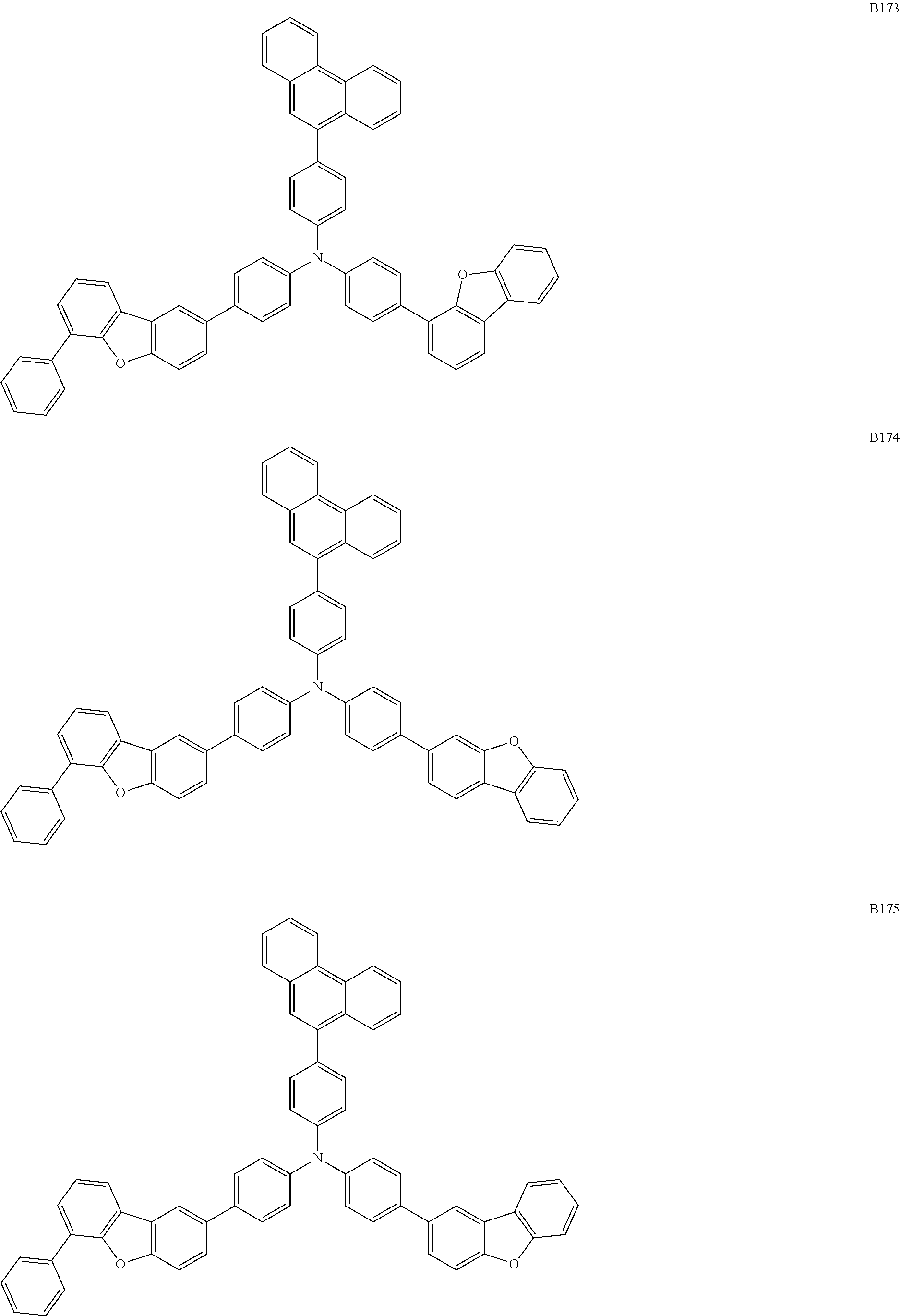 [SMILES: c1ccc(-c2cccc3c2oc2ccc(-c4ccc(N(c5ccc(-c6cc7ccccc7c7ccccc67)cc5)c5ccc(-c6cccc7c6oc6ccccc67)cc5)cc4)cc23)cc1 ; c1ccc(-c2cccc3c2oc2ccc(-c4ccc(N(c5ccc(-c6ccc7c(c6)oc6ccccc67)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7oc8ccccc8c7c6)cc5)c5ccc(-c6cc7ccccc7c7ccccc67)cc5)cc4)cc23)cc1] 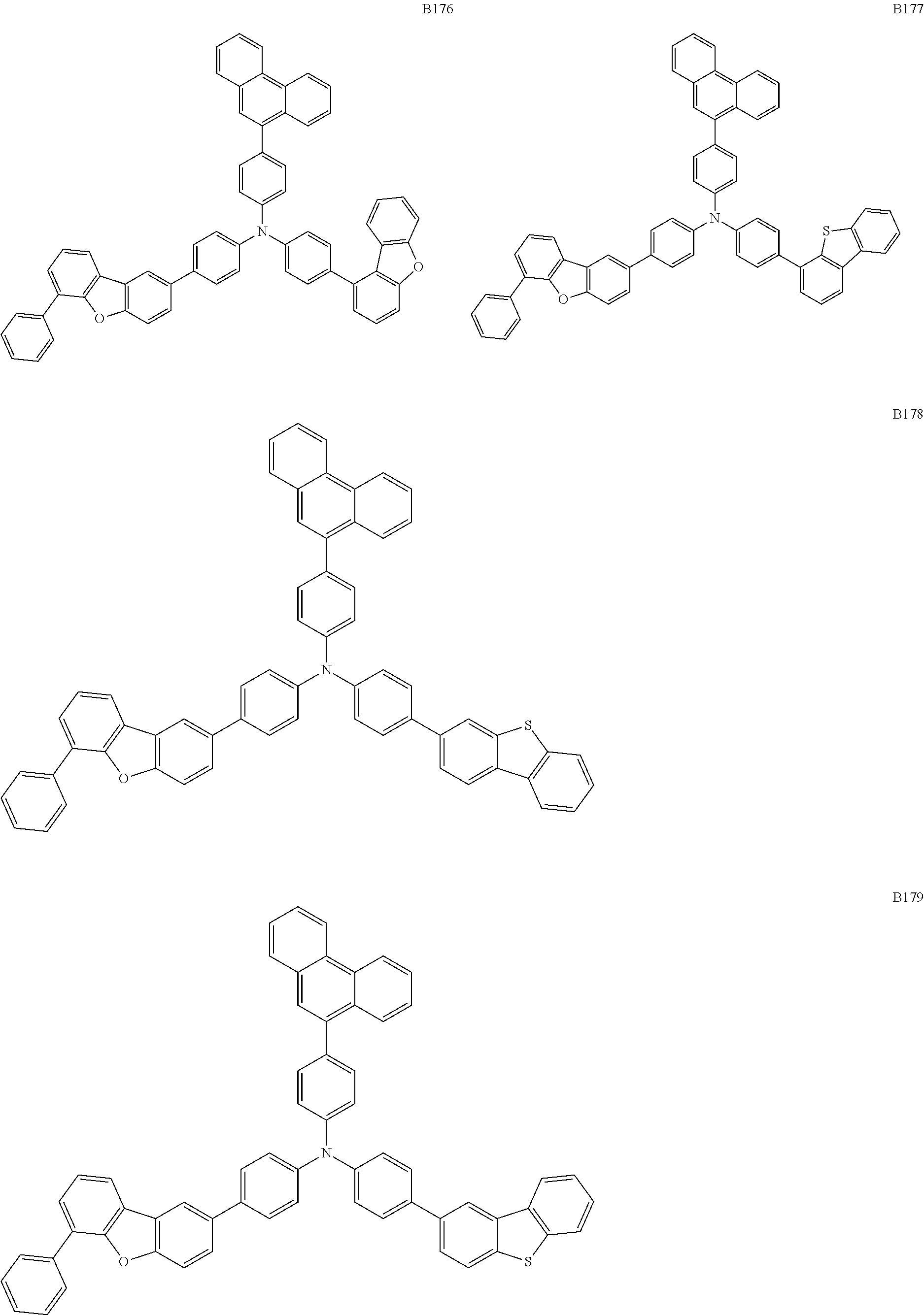 [SMILES: c1ccc(-c2cccc3c2oc2ccc(-c4ccc(N(c5ccc(-c6cc7ccccc7c7ccccc67)cc5)c5ccc(-c6cccc7oc8ccccc8c67)cc5)cc4)cc23)cc1 ; c1ccc(-c2cccc3c2oc2ccc(-c4ccc(N(c5ccc(-c6cc7ccccc7c7ccccc67)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7sc8ccccc8c7c6)cc5)c5ccc(-c6cc7ccccc7c7ccccc67)cc5)cc4)cc23)cc1] 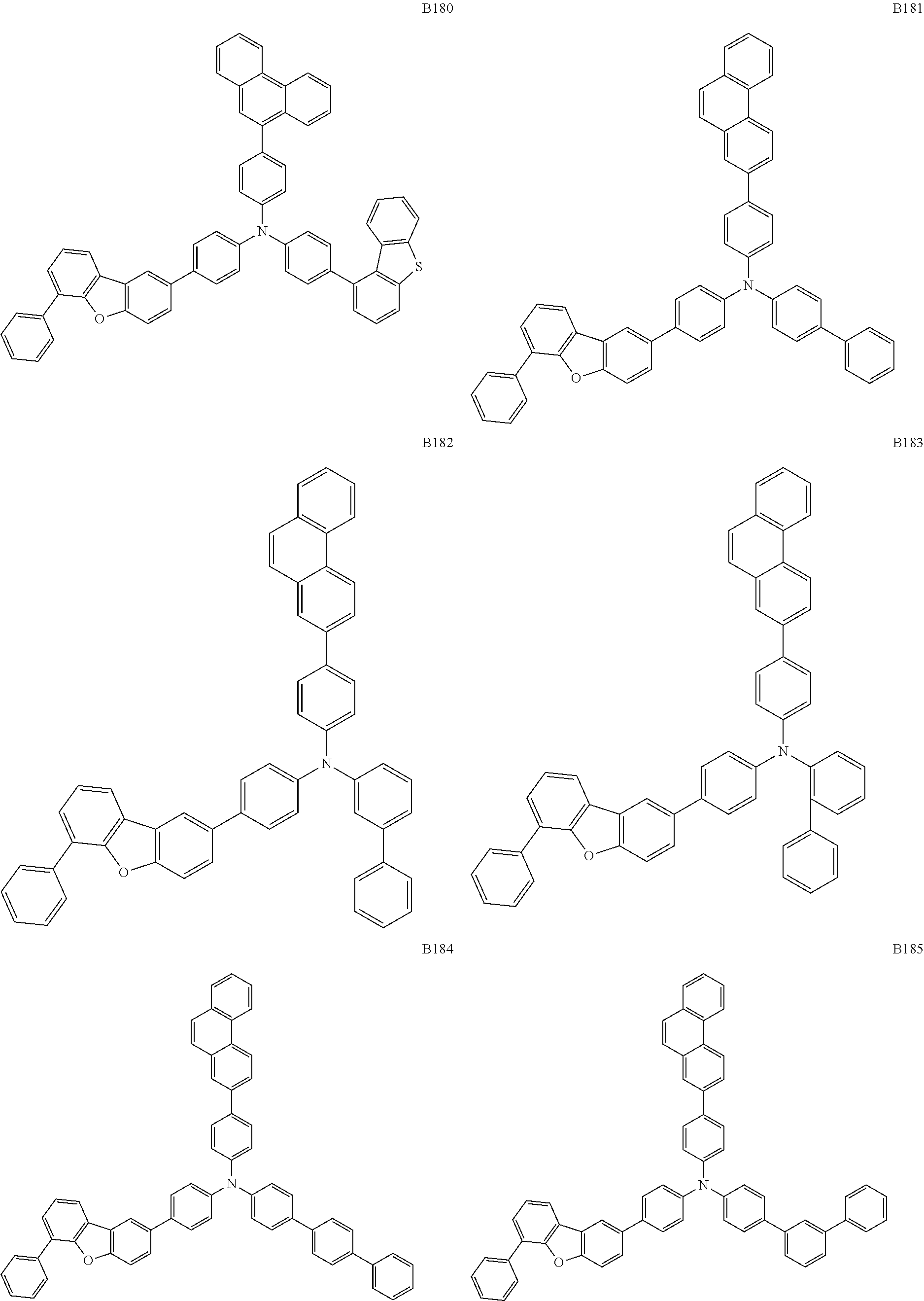 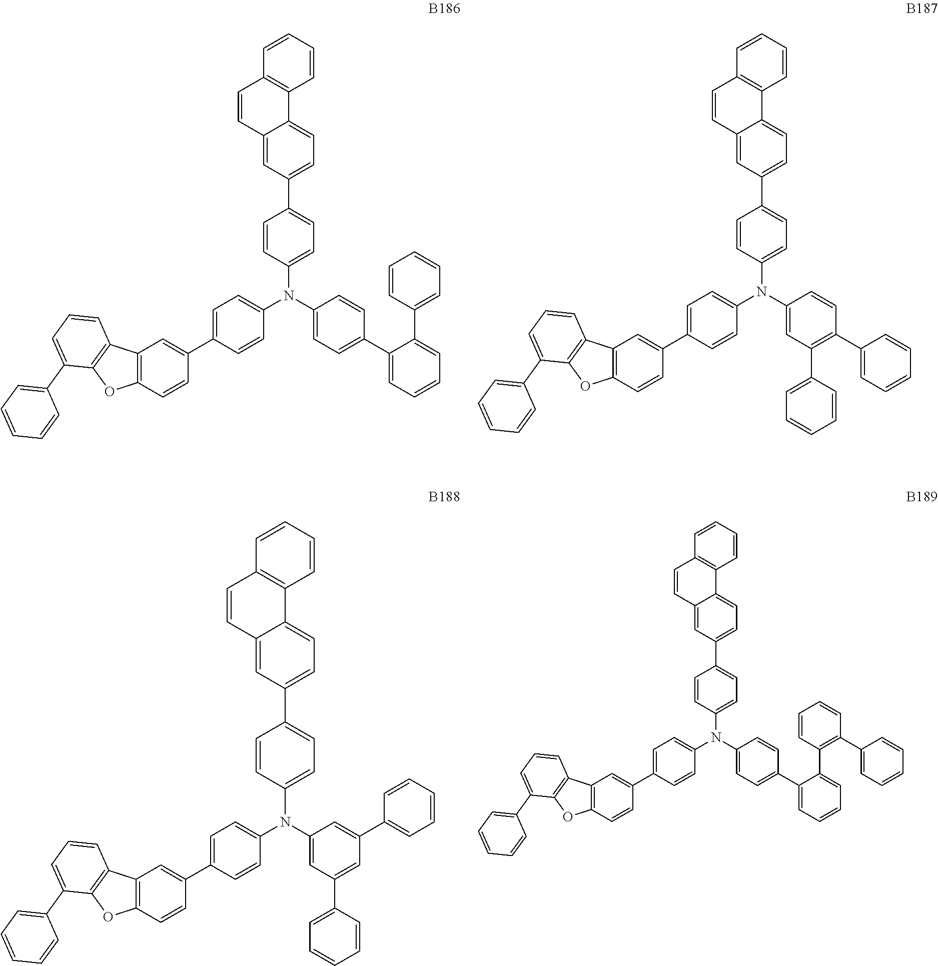 [SMILES: c1ccc(-c2ccccc2-c2ccc(N(c3ccc(-c4ccc5c(ccc6ccccc65)c4)cc3)c3ccc(-c4ccc5oc6c(-c7ccccc7)cccc6c5c4)cc3)cc2)cc1 ; c1ccc(-c2ccc(N(c3ccc(-c4ccc5c(ccc6ccccc65)c4)cc3)c3ccc(-c4ccc5oc6c(-c7ccccc7)cccc6c5c4)cc3)cc2-c2ccccc2)cc1 ; c1ccc(-c2cc(-c3ccccc3)cc(N(c3ccc(-c4ccc5c(ccc6ccccc65)c4)cc3)c3ccc(-c4ccc5oc6c(-c7ccccc7)cccc6c5c4)cc3)c2)cc1 ; c1ccc(-c2ccccc2-c2ccccc2-c2ccc(N(c3ccc(-c4ccc5c(ccc6ccccc65)c4)cc3)c3ccc(-c4ccc5oc6c(-c7ccccc7)cccc6c5c4)cc3)cc2)cc1] 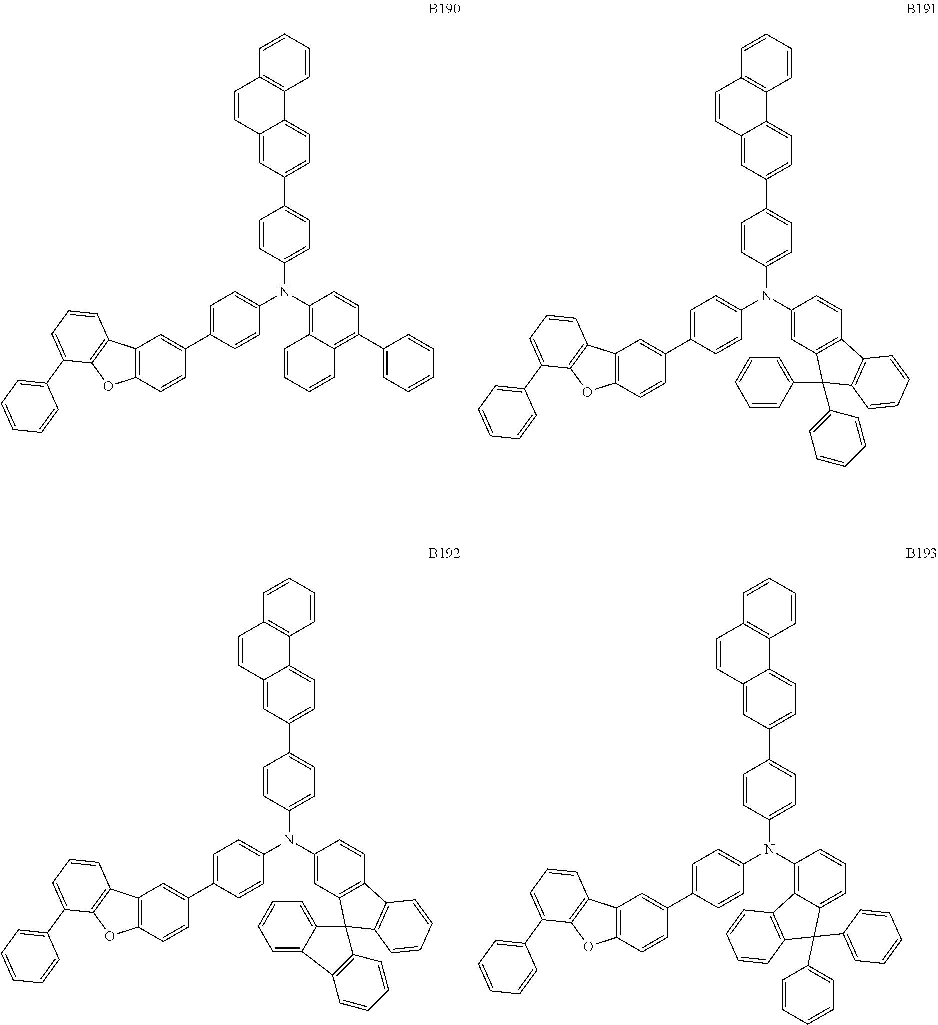 [SMILES: c1ccc(-c2ccc(N(c3ccc(-c4ccc5c(ccc6ccccc65)c4)cc3)c3ccc(-c4ccc5oc6c(-c7ccccc7)cccc6c5c4)cc3)c3ccccc23)cc1 ; c1ccc(-c2cccc3c2oc2ccc(-c4ccc(N(c5ccc(-c6ccc7c(ccc8ccccc87)c6)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6ccc7c(ccc8ccccc87)c6)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6ccc7c(ccc8ccccc87)c6)cc5)c5cccc6c5-c5ccccc5C6(c5ccccc5)c5ccccc5)cc4)cc23)cc1] 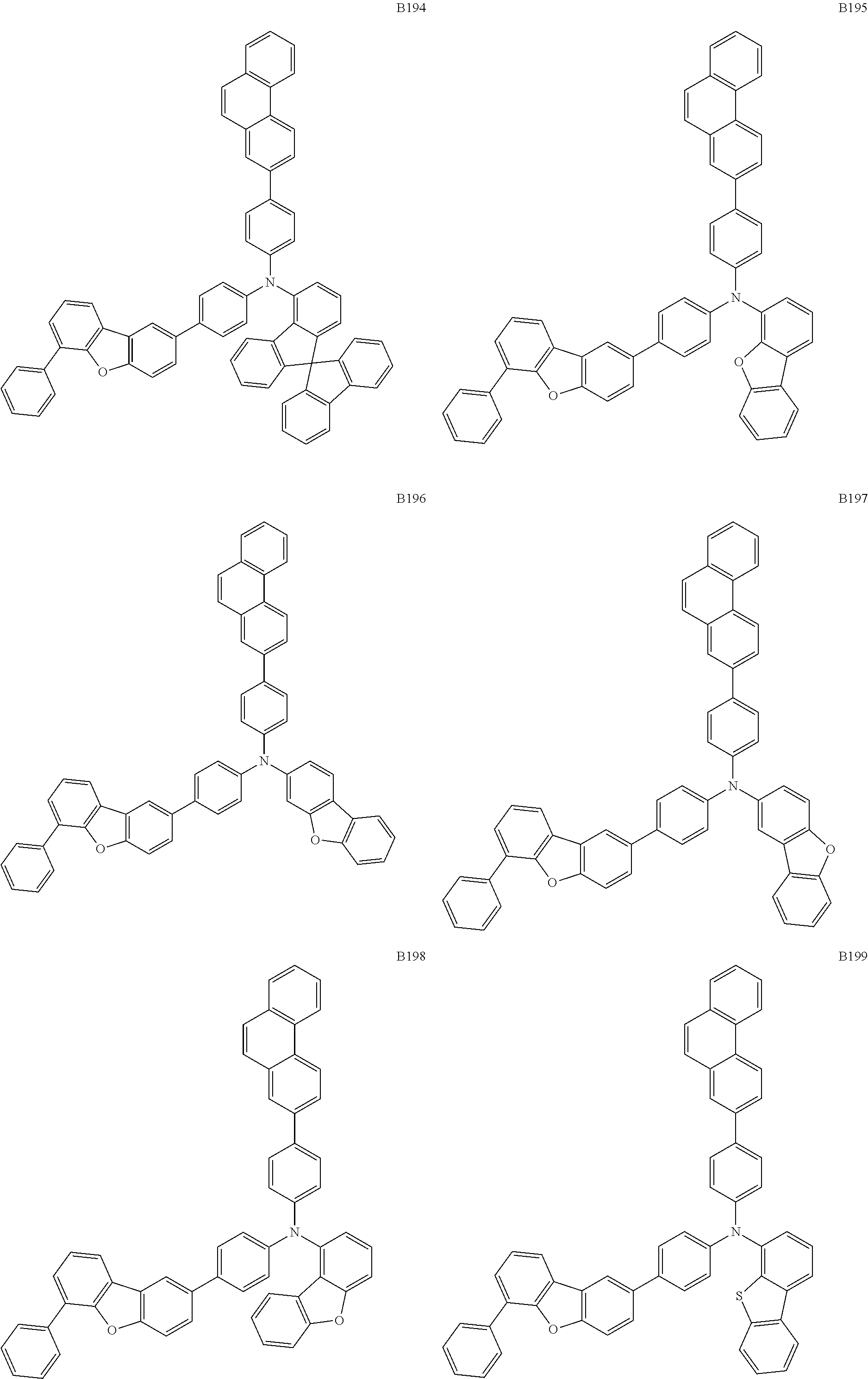 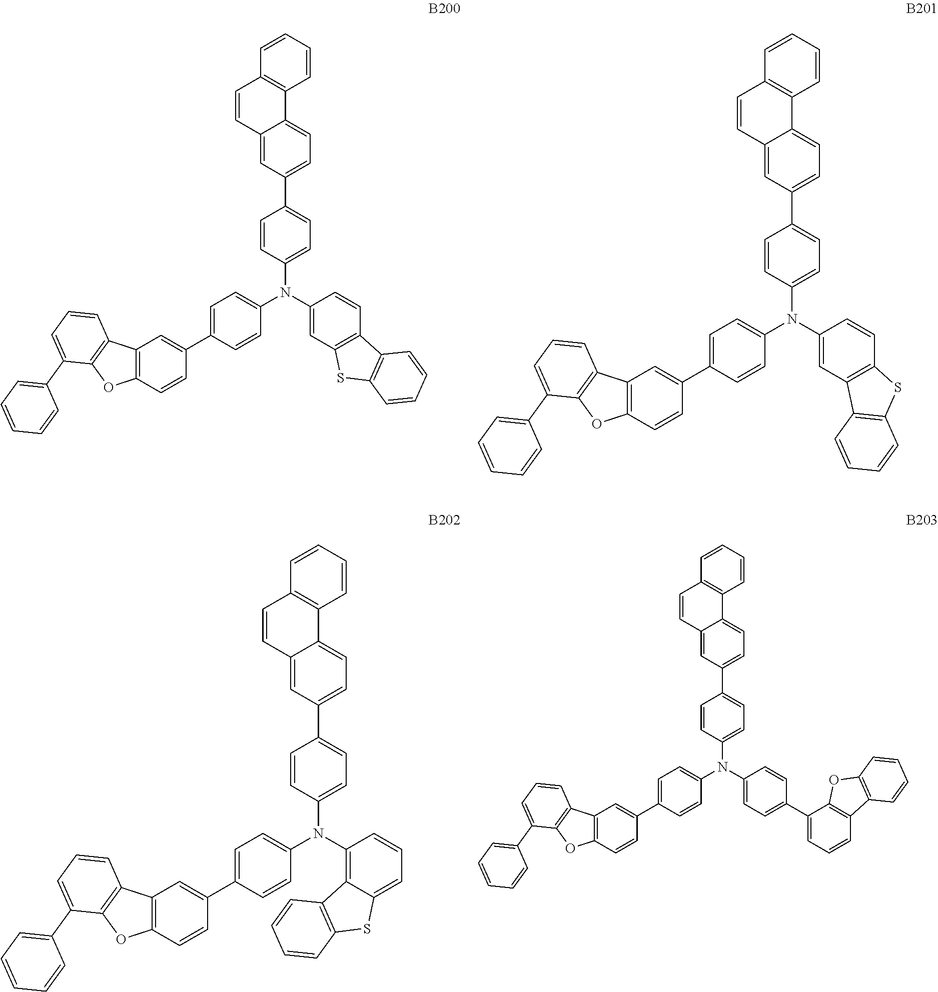 [SMILES: c1ccc(-c2cccc3c2oc2ccc(-c4ccc(N(c5ccc(-c6ccc7c(ccc8ccccc87)c6)cc5)c5ccc6c(c5)sc5ccccc56)cc4)cc23)cc1 ; c1ccc(-c2cccc3c2oc2ccc(-c4ccc(N(c5ccc(-c6ccc7c(ccc8ccccc87)c6)cc5)c5ccc6sc7ccccc7c6c5)cc4)cc23)cc1 ; c1ccc(-c2cccc3c2oc2ccc(-c4ccc(N(c5ccc(-c6ccc7c(ccc8ccccc87)c6)cc5)c5cccc6sc7ccccc7c56)cc4)cc23)cc1 ; c1ccc(-c2cccc3c2oc2ccc(-c4ccc(N(c5ccc(-c6ccc7c(ccc8ccccc87)c6)cc5)c5ccc(-c6cccc7c6oc6ccccc67)cc5)cc4)cc23)cc1] 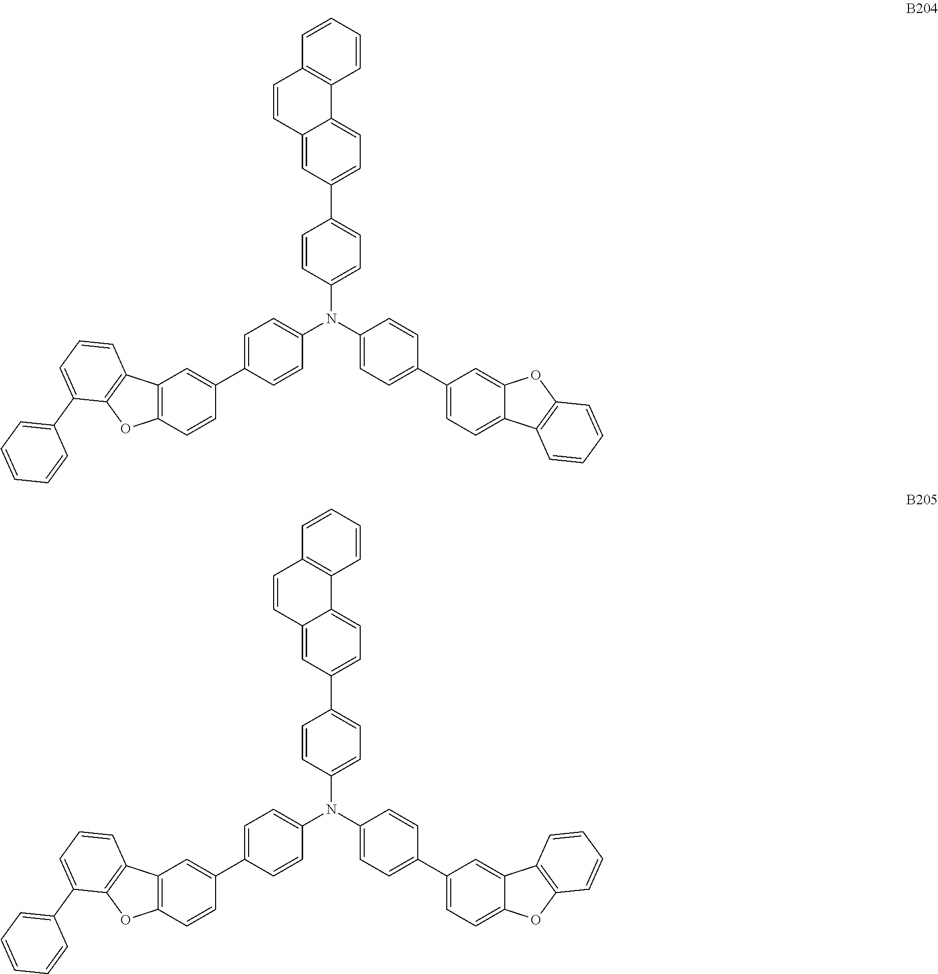 [SMILES: c1ccc(-c2cccc3c2oc2ccc(-c4ccc(N(c5ccc(-c6ccc7c(ccc8ccccc87)c6)cc5)c5ccc(-c6ccc7c(c6)oc6ccccc67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6ccc7oc8ccccc8c7c6)cc5)cc4)cc23)cc1] 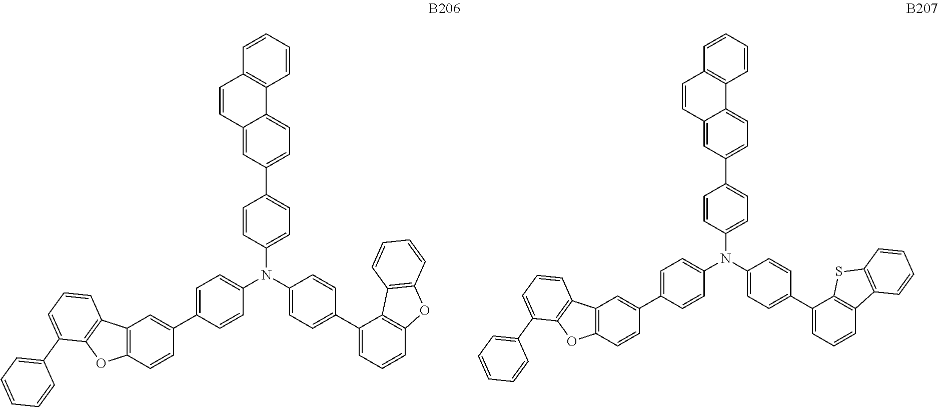 [SMILES: c1ccc(-c2cccc3c2oc2ccc(-c4ccc(N(c5ccc(-c6ccc7c(ccc8ccccc87)c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6cccc7c6sc6ccccc67)cc5)cc4)cc23)cc1] 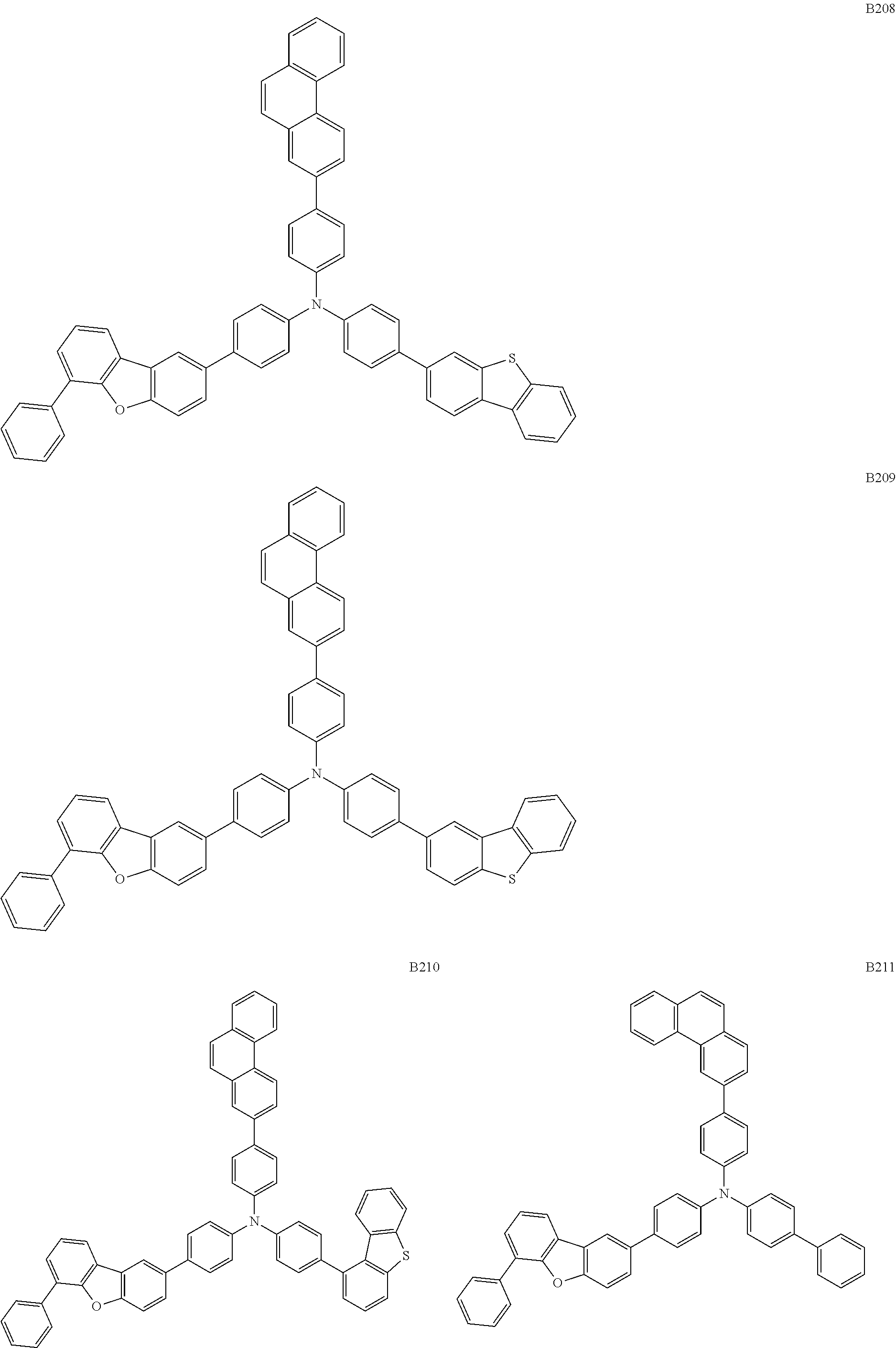 [SMILES: c1ccc(-c2cccc3c2oc2ccc(-c4ccc(N(c5ccc(-c6ccc7c(ccc8ccccc87)c6)cc5)c5ccc(-c6ccc7c(c6)sc6ccccc67)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6ccc7sc8ccccc8c7c6)cc5)cc4)cc23)cc1 ; c1ccc(-c2cccc3c2oc2ccc(-c4ccc(N(c5ccc(-c6ccc7c(ccc8ccccc87)c6)cc5)c5ccc(-c6cccc7sc8ccccc8c67)cc5)cc4)cc23)cc1 ; c1ccc(-c2ccc(N(c3ccc(-c4ccc5ccc6ccccc6c5c4)cc3)c3ccc(-c4ccc5oc6c(-c7ccccc7)cccc6c5c4)cc3)cc2)cc1] 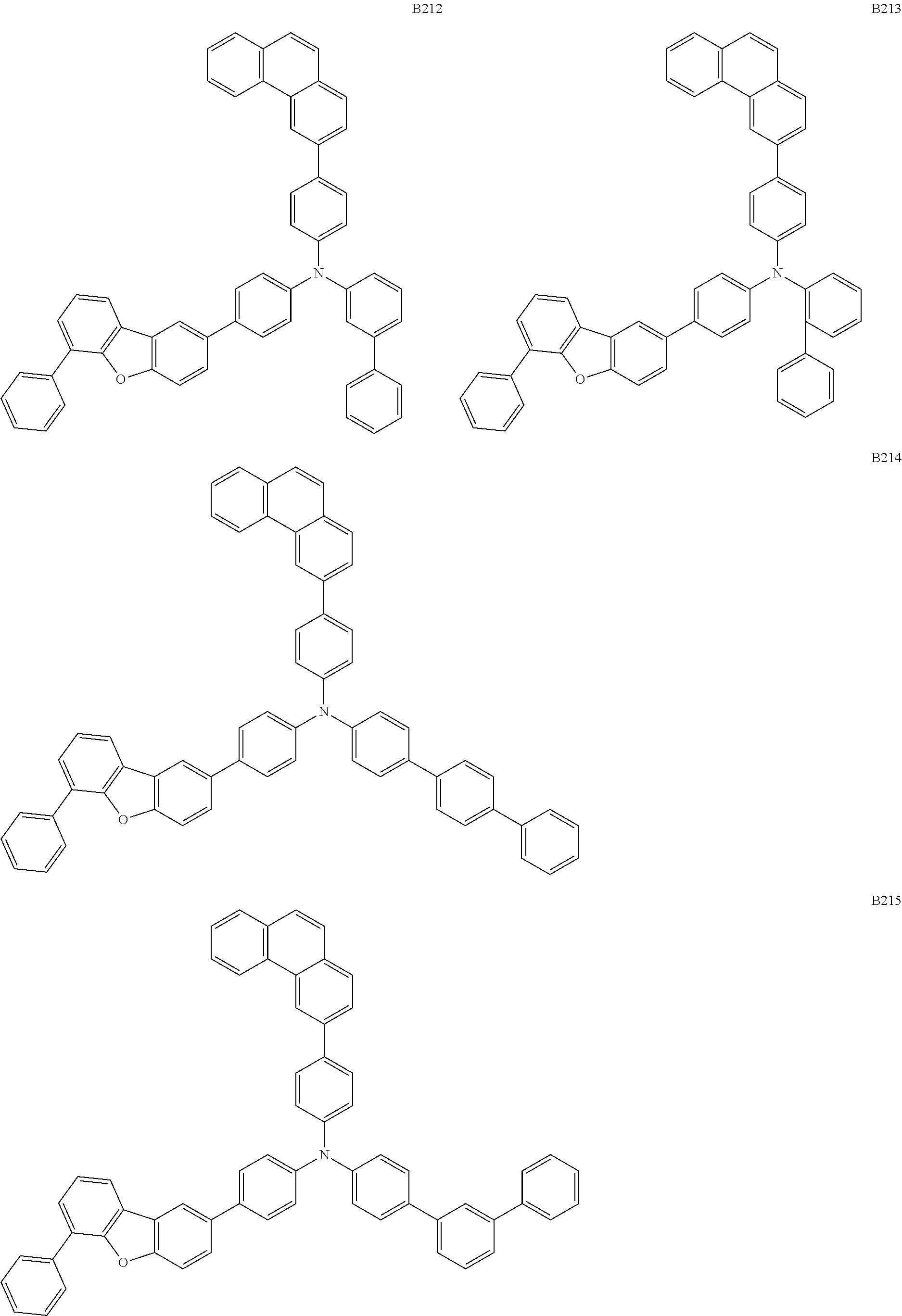 [SMILES: c1ccc(-c2cccc(N(c3ccc(-c4ccc5ccc6ccccc6c5c4)cc3)c3ccc(-c4ccc5oc6c(-c7ccccc7)cccc6c5c4)cc3)c2)cc1 ; c1ccc(-c2ccccc2N(c2ccc(-c3ccc4ccc5ccccc5c4c3)cc2)c2ccc(-c3ccc4oc5c(-c6ccccc6)cccc5c4c3)cc2)cc1 ; c1ccc(-c2ccc(-c3ccc(N(c4ccc(-c5ccc6ccc7ccccc7c6c5)cc4)c4ccc(-c5ccc6oc7c(-c8ccccc8)cccc7c6c5)cc4)cc3)cc2)cc1 ; c1ccc(-c2cccc(-c3ccc(N(c4ccc(-c5ccc6ccc7ccccc7c6c5)cc4)c4ccc(-c5ccc6oc7c(-c8ccccc8)cccc7c6c5)cc4)cc3)c2)cc1] 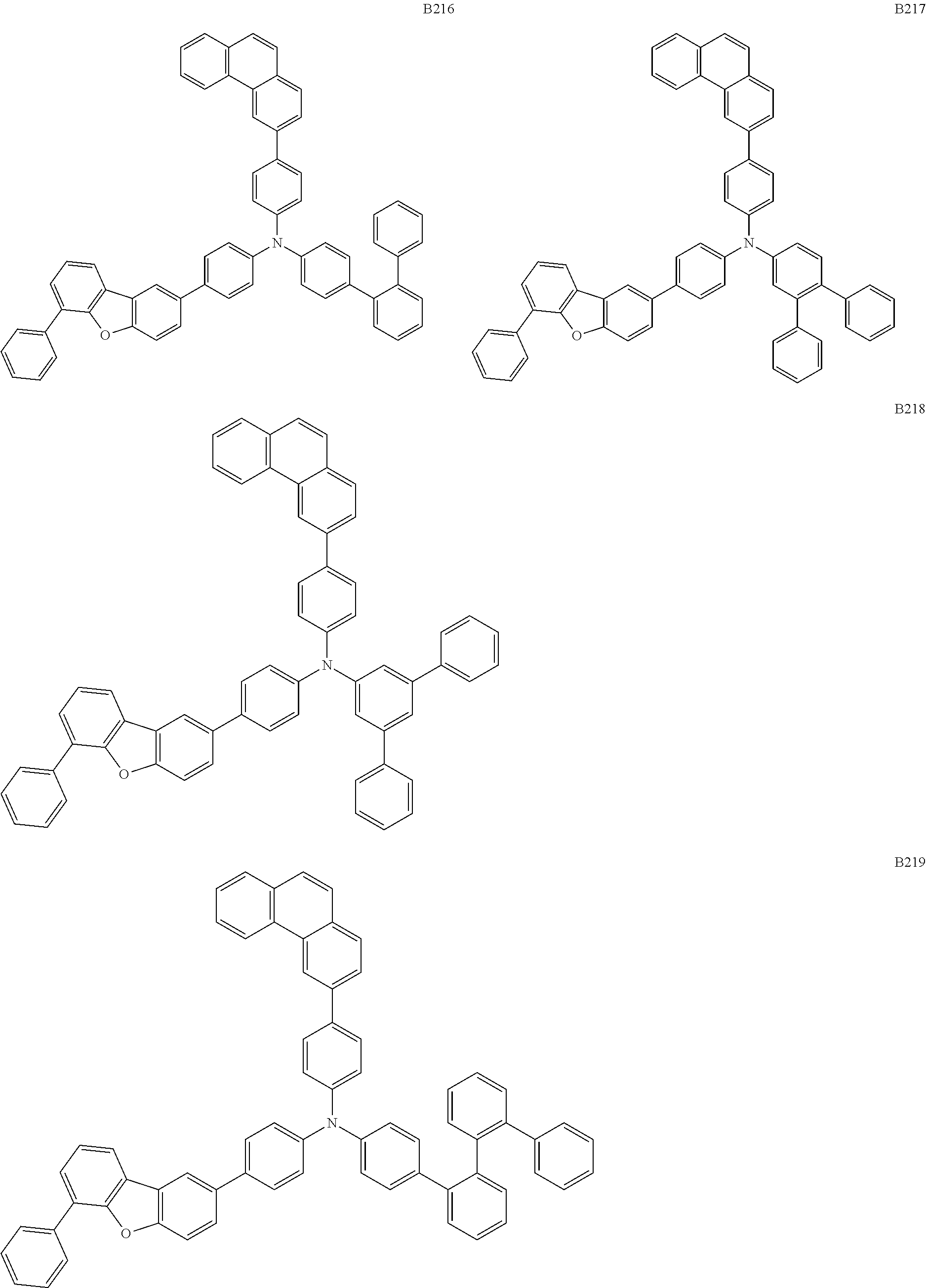 [SMILES: c1ccc(-c2ccccc2-c2ccc(N(c3ccc(-c4ccc5ccc6ccccc6c5c4)cc3)c3ccc(-c4ccc5oc6c(-c7ccccc7)cccc6c5c4)cc3)cc2)cc1 ; c1ccc(-c2ccc(N(c3ccc(-c4ccc5ccc6ccccc6c5c4)cc3)c3ccc(-c4ccc5oc6c(-c7ccccc7)cccc6c5c4)cc3)cc2-c2ccccc2)cc1 ; c1ccc(-c2cc(-c3ccccc3)cc(N(c3ccc(-c4ccc5ccc6ccccc6c5c4)cc3)c3ccc(-c4ccc5oc6c(-c7ccccc7)cccc6c5c4)cc3)c2)cc1 ; c1ccc(-c2ccccc2-c2ccccc2-c2ccc(N(c3ccc(-c4ccc5ccc6ccccc6c5c4)cc3)c3ccc(-c4ccc5oc6c(-c7ccccc7)cccc6c5c4)cc3)cc2)cc1] 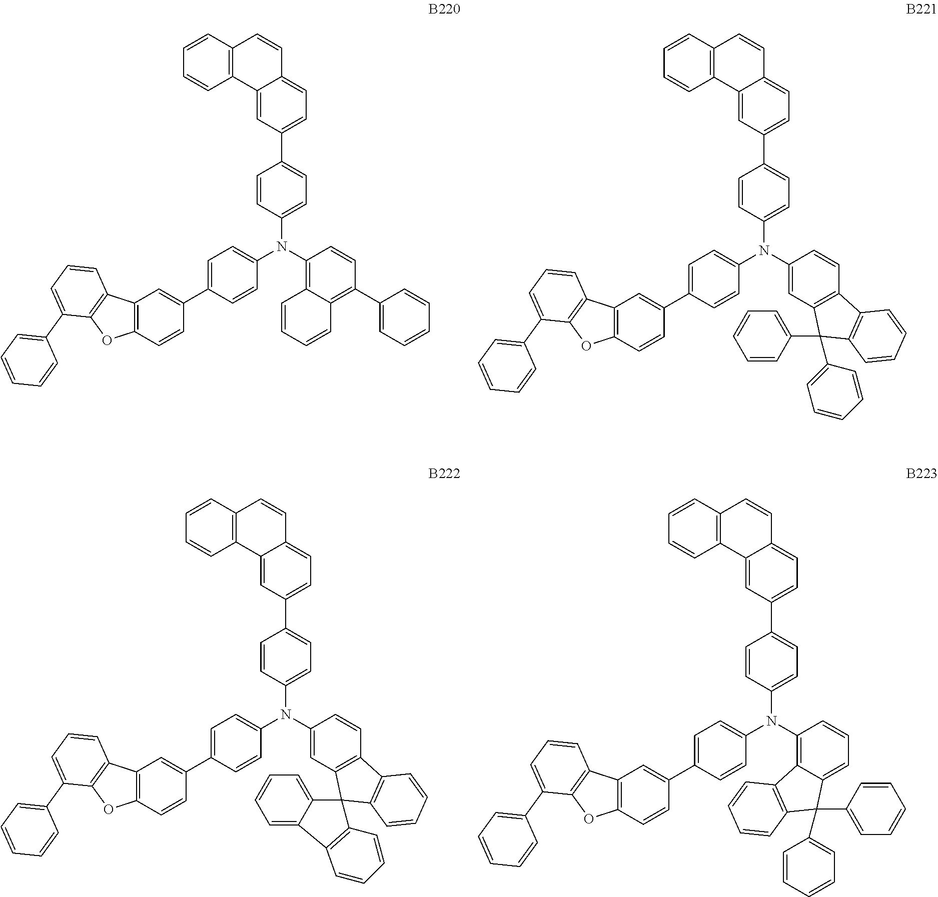 [SMILES: c1ccc(-c2ccc(N(c3ccc(-c4ccc5ccc6ccccc6c5c4)cc3)c3ccc(-c4ccc5oc6c(-c7ccccc7)cccc6c5c4)cc3)c3ccccc23)cc1 ; c1ccc(-c2cccc3c2oc2ccc(-c4ccc(N(c5ccc(-c6ccc7ccc8ccccc8c7c6)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6ccc7ccc8ccccc8c7c6)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6ccc7ccc8ccccc8c7c6)cc5)c5cccc6c5-c5ccccc5C6(c5ccccc5)c5ccccc5)cc4)cc23)cc1] 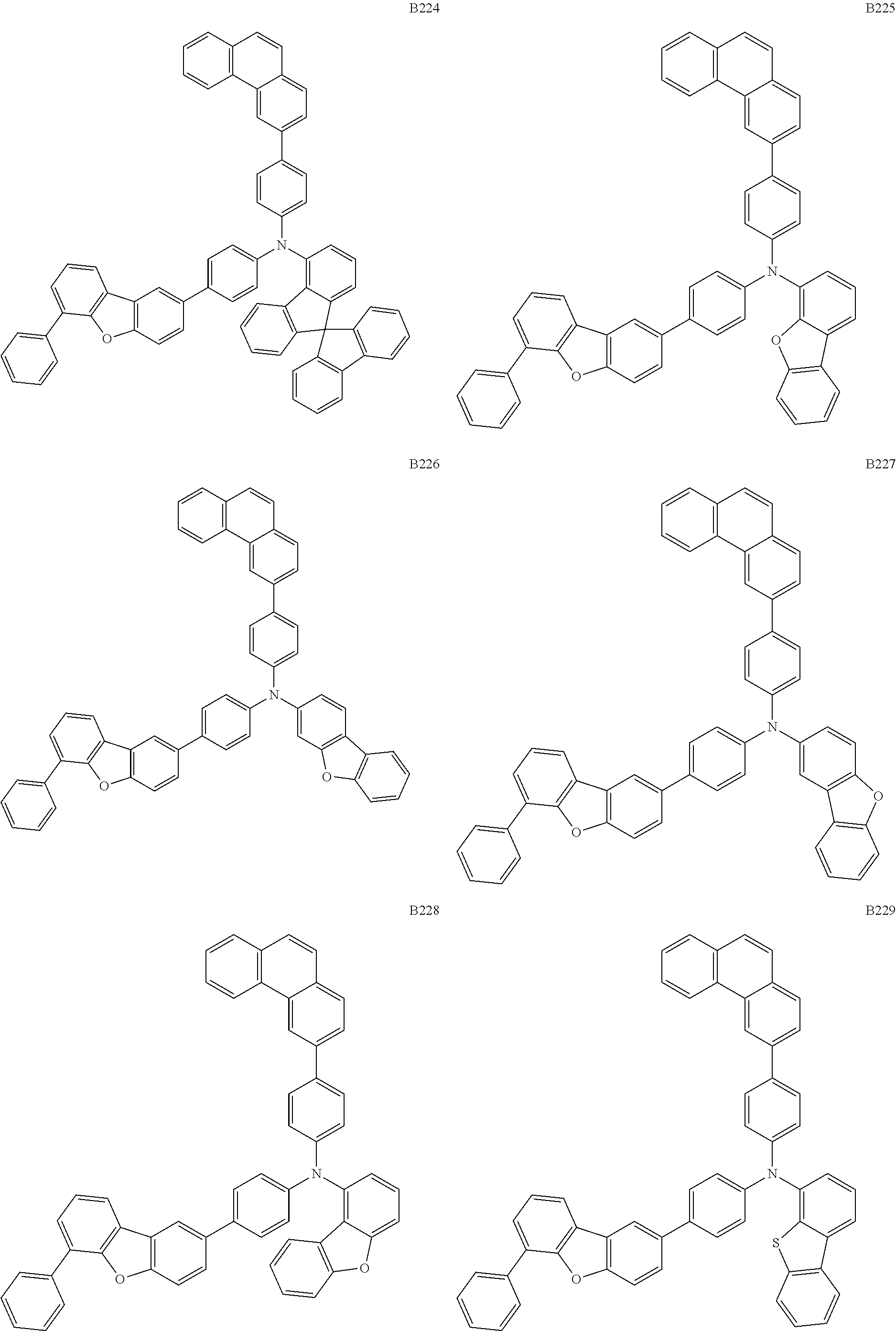 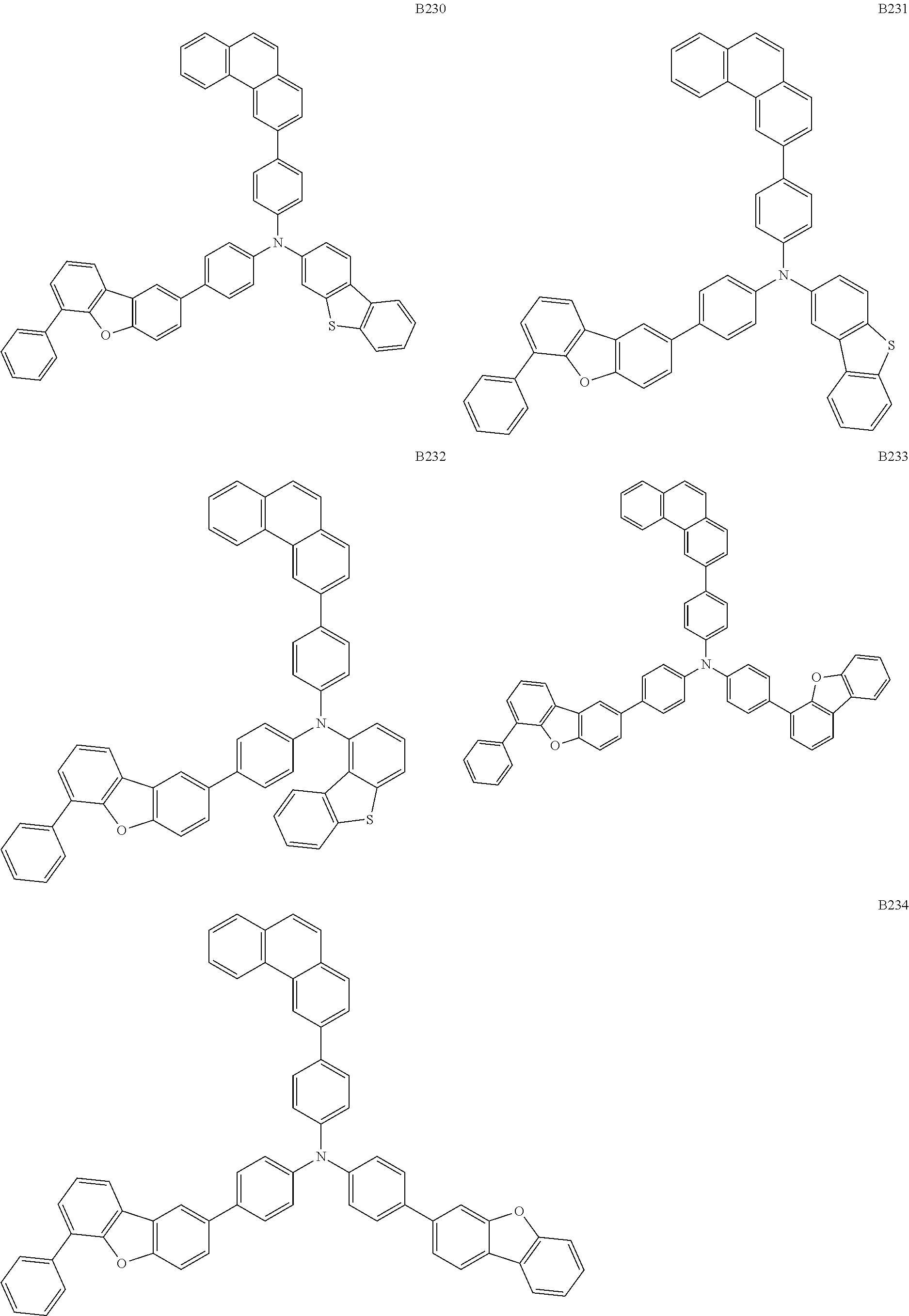 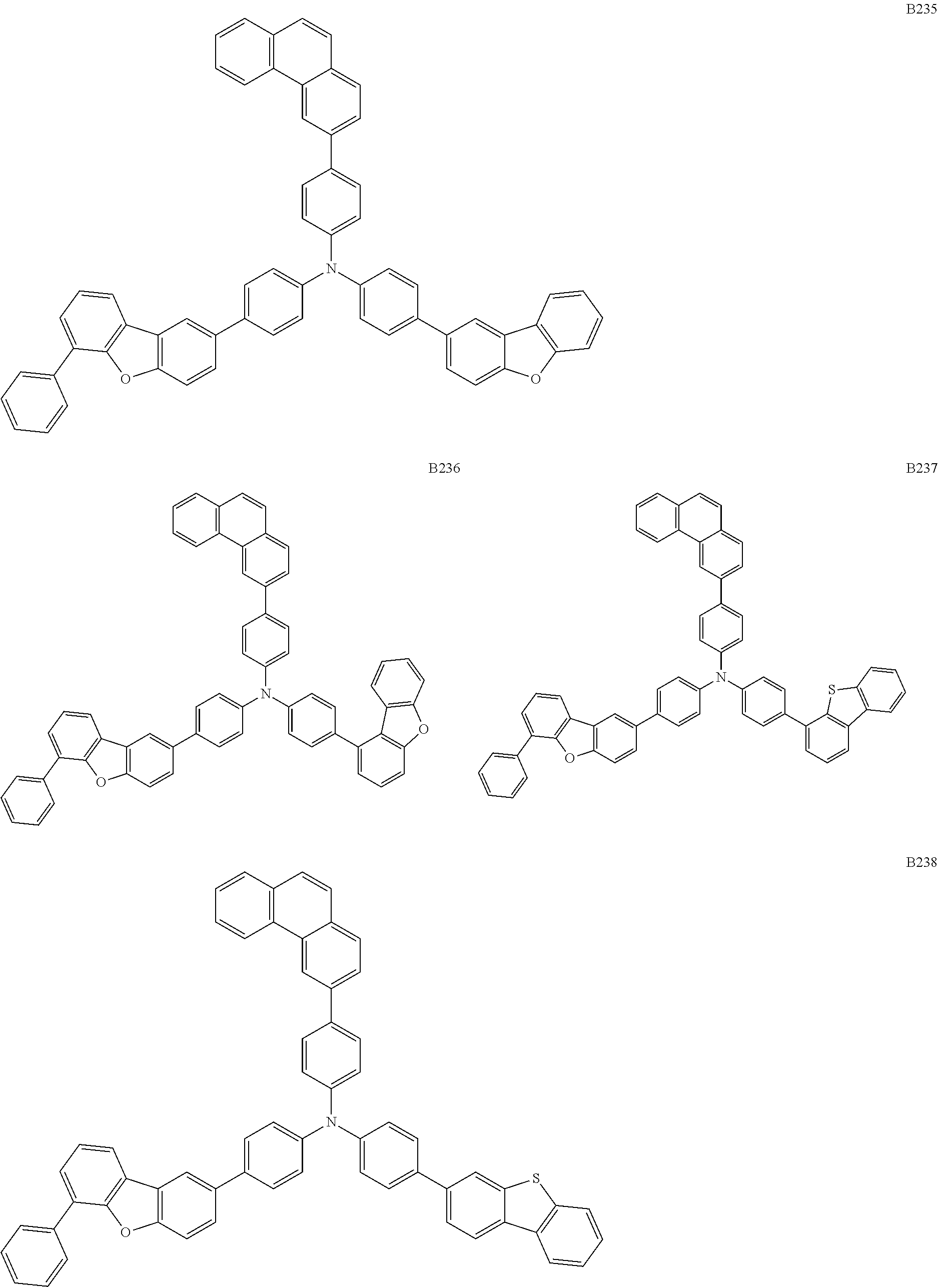 [SMILES: c1ccc(-c2cccc3c2oc2ccc(-c4ccc(N(c5ccc(-c6ccc7ccc8ccccc8c7c6)cc5)c5ccc(-c6ccc7oc8ccccc8c7c6)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6ccc7ccc8ccccc8c7c6)cc5)cc4)cc23)cc1] 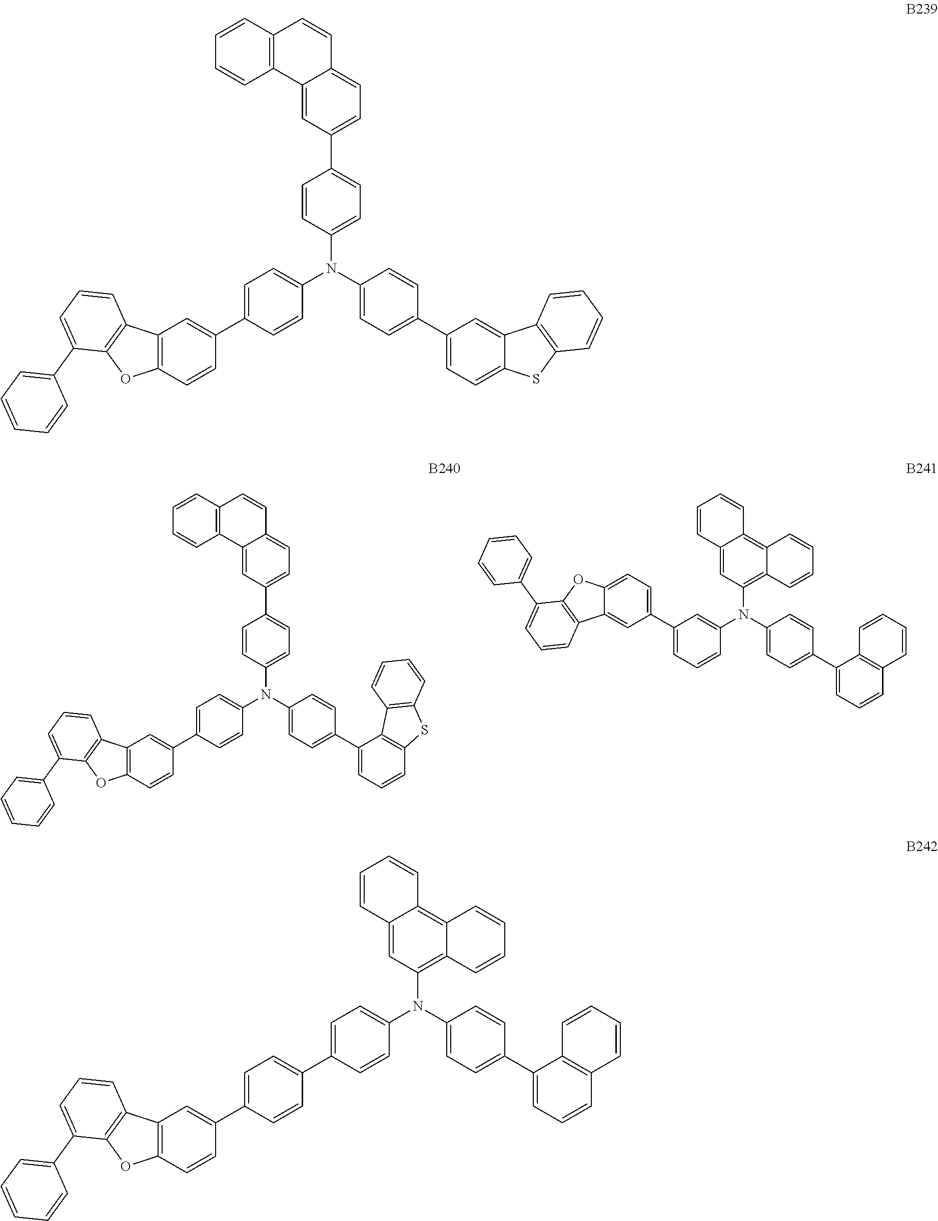 [SMILES: c1ccc(-c2cccc3c2oc2ccc(-c4ccc(N(c5ccc(-c6ccc7ccc8ccccc8c7c6)cc5)c5ccc(-c6ccc7sc8ccccc8c7c6)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7sc8ccccc8c67)cc5)cc4)cc23)cc1 ; c1ccc(-c2cccc3c2oc2ccc(-c4cccc(N(c5ccc(-c6cccc7ccccc67)cc5)c5cc6ccccc6c6ccccc56)c4)cc23)cc1 ; c1ccc(-c2cccc3c2oc2ccc(-c4ccc(-c5ccc(N(c6ccc(-c7cccc8ccccc78)cc6)c6cc7ccccc7c7ccccc67)cc5)cc4)cc23)cc1] 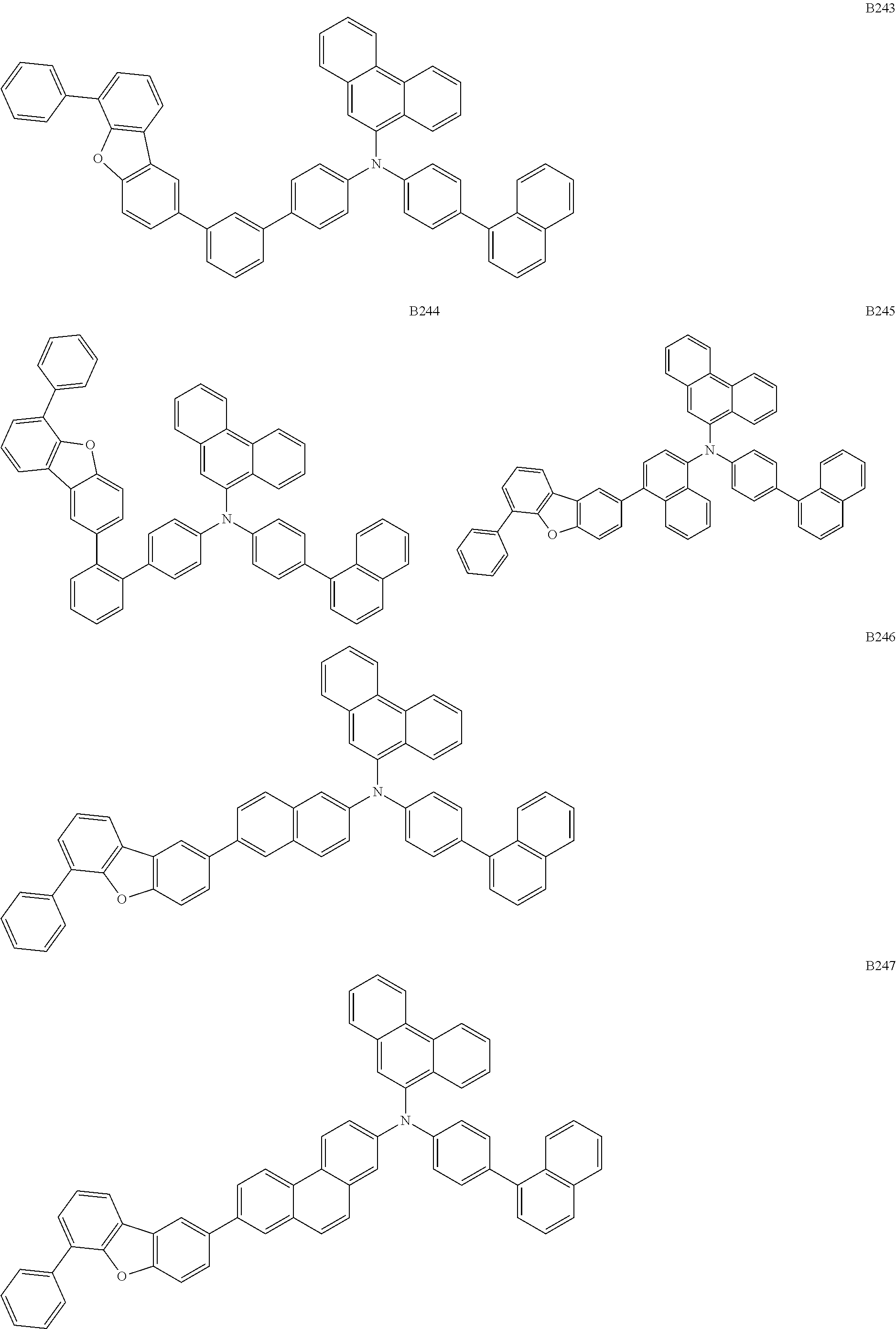 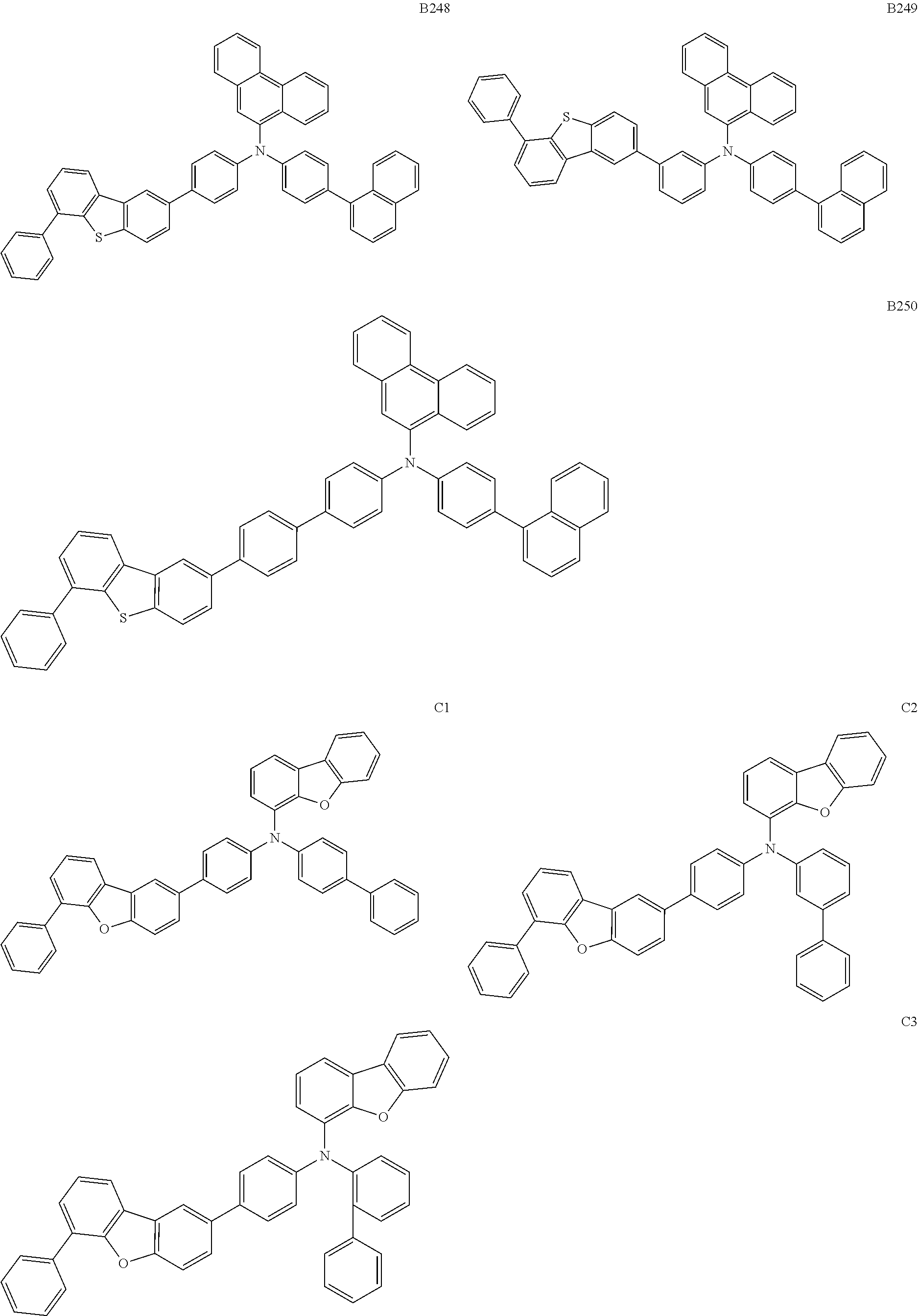 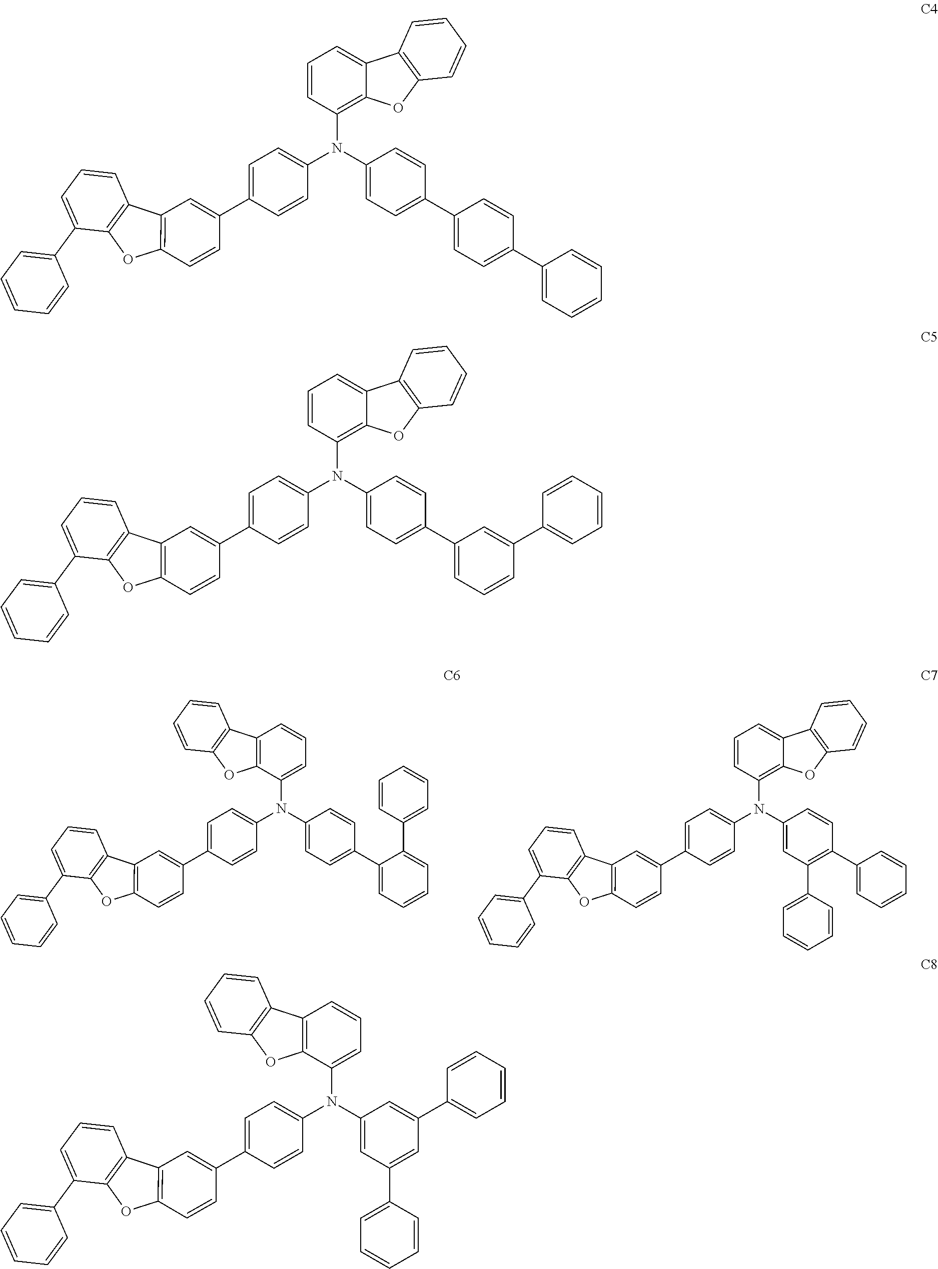 [SMILES: c1ccc(-c2ccc(-c3ccc(N(c4ccc(-c5ccc6oc7c(-c8ccccc8)cccc7c6c5)cc4)c4cccc5c4oc4ccccc45)cc3)cc2)cc1 ; c1ccc(-c2cccc(-c3ccc(N(c4ccc(-c5ccc6oc7c(-c8ccccc8)cccc7c6c5)cc4)c4cccc5c4oc4ccccc45)cc3)c2)cc1 ; c1ccc(-c2ccccc2-c2ccc(N(c3ccc(-c4ccc5oc6c(-c7ccccc7)cccc6c5c4)cc3)c3cccc4c3oc3ccccc34)cc2)cc1 ; c1ccc(-c2ccc(N(c3ccc(-c4ccc5oc6c(-c7ccccc7)cccc6c5c4)cc3)c3cccc4c3oc3ccccc34)cc2-c2ccccc2)cc1 ; c1ccc(-c2cc(-c3ccccc3)cc(N(c3ccc(-c4ccc5oc6c(-c7ccccc7)cccc6c5c4)cc3)c3cccc4c3oc3ccccc34)c2)cc1] 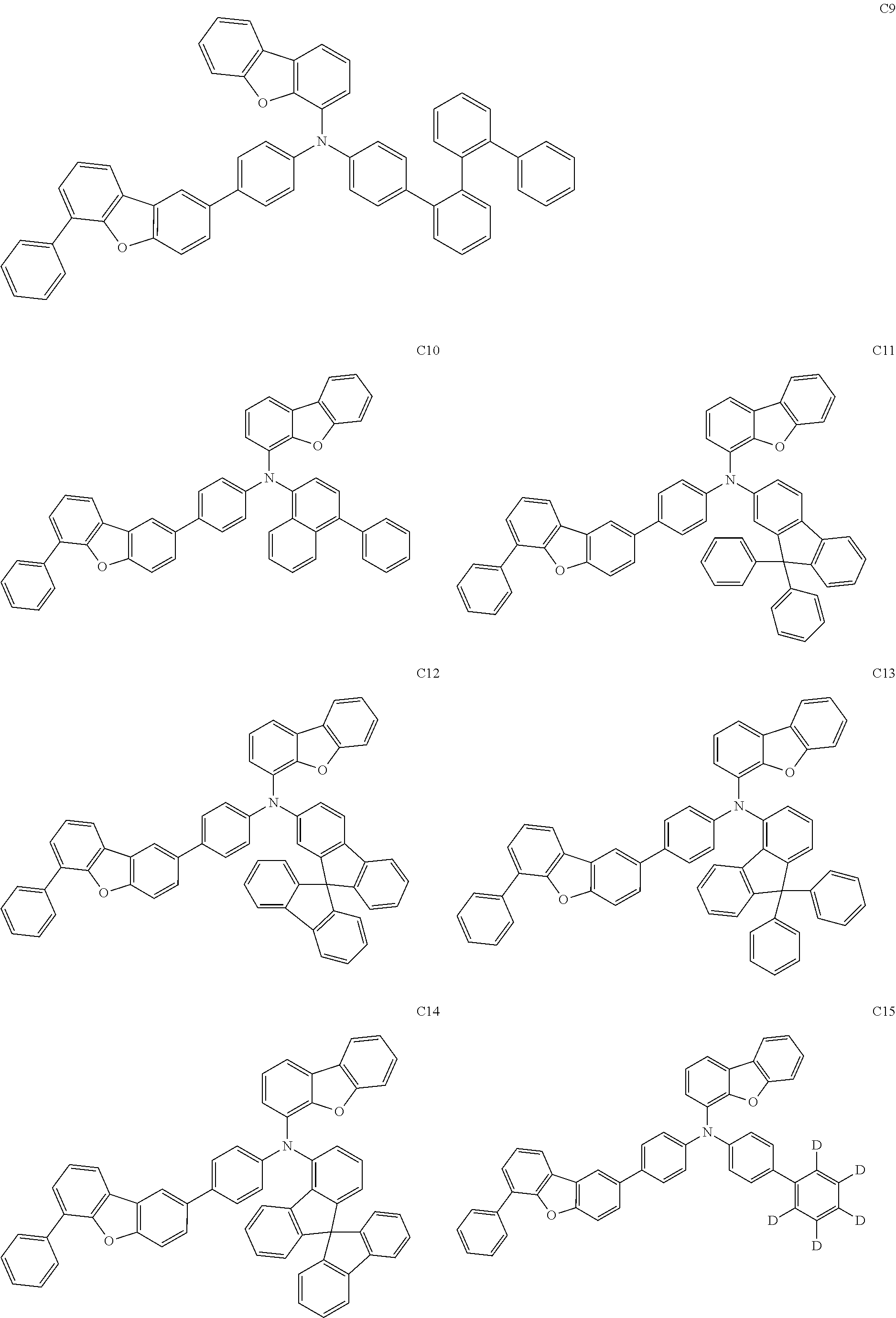 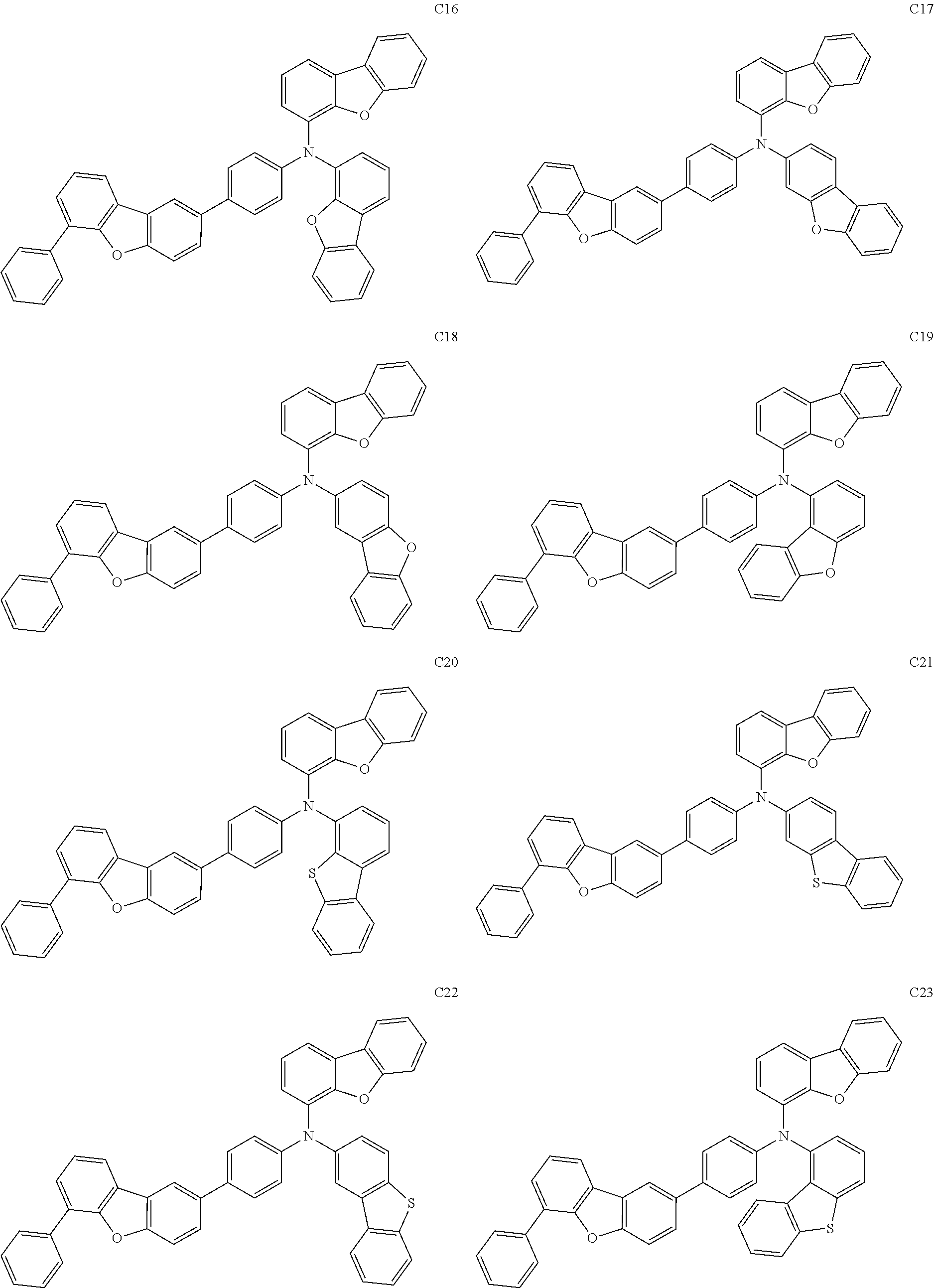 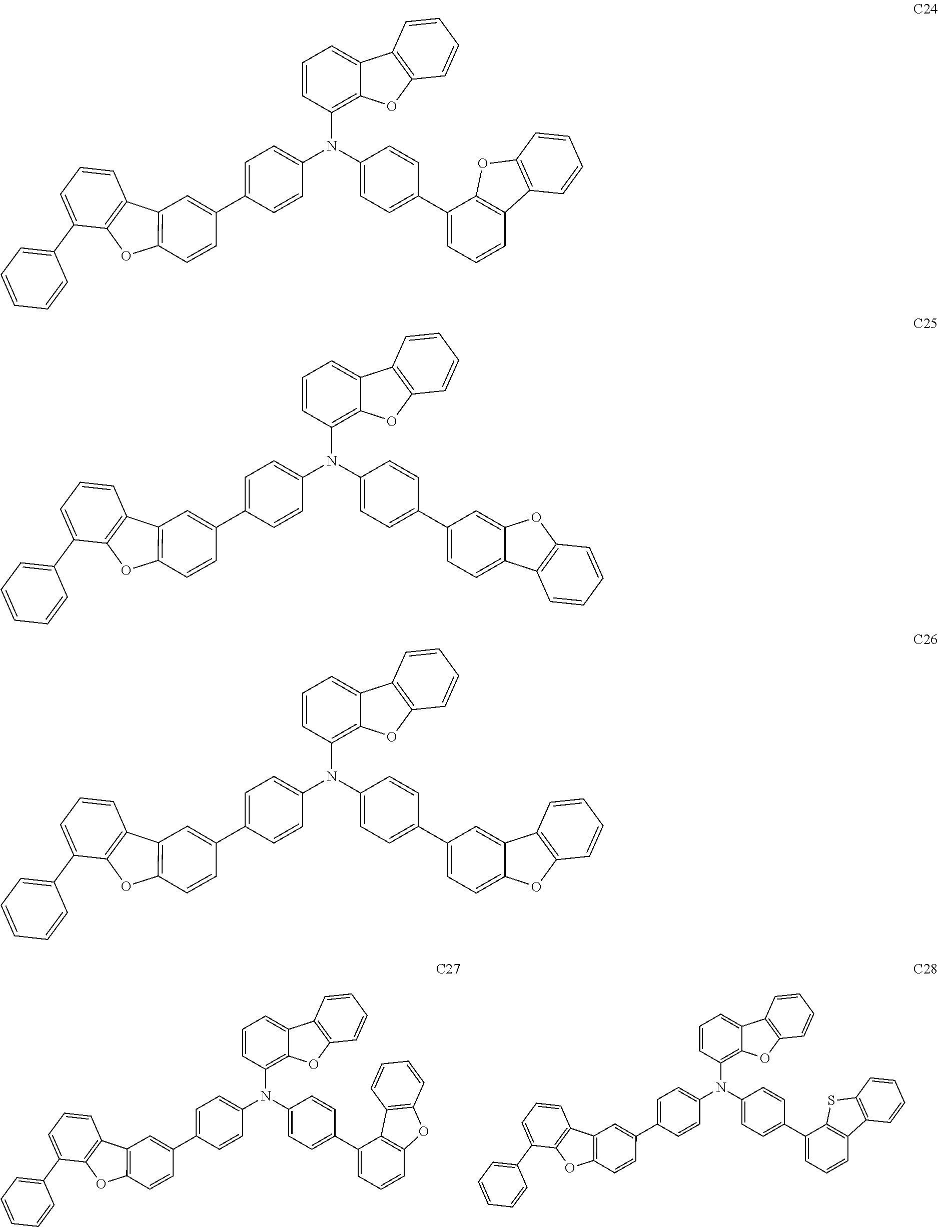 [SMILES: c1ccc(-c2cccc3c2oc2ccc(-c4ccc(N(c5ccc(-c6cccc7c6oc6ccccc67)cc5)c5cccc6c5oc5ccccc56)cc4)cc23)cc1 ; c1ccc(-c2cccc3c2oc2ccc(-c4ccc(N(c5ccc(-c6ccc7c(c6)oc6ccccc67)cc5)c5cccc6c5oc5ccccc56)cc4)cc23)cc1 ; c1ccc(-c2cccc3c2oc2ccc(-c4ccc(N(c5ccc(-c6ccc7oc8ccccc8c7c6)cc5)c5cccc6c5oc5ccccc56)cc4)cc23)cc1 ; c1ccc(-c2cccc3c2oc2ccc(-c4ccc(N(c5ccc(-c6cccc7oc8ccccc8c67)cc5)c5cccc6c5oc5ccccc56)cc4)cc23)cc1 ; c1ccc(-c2cccc3c2oc2ccc(-c4ccc(N(c5ccc(-c6cccc7c6sc6ccccc67)cc5)c5cccc6c5oc5ccccc56)cc4)cc23)cc1] 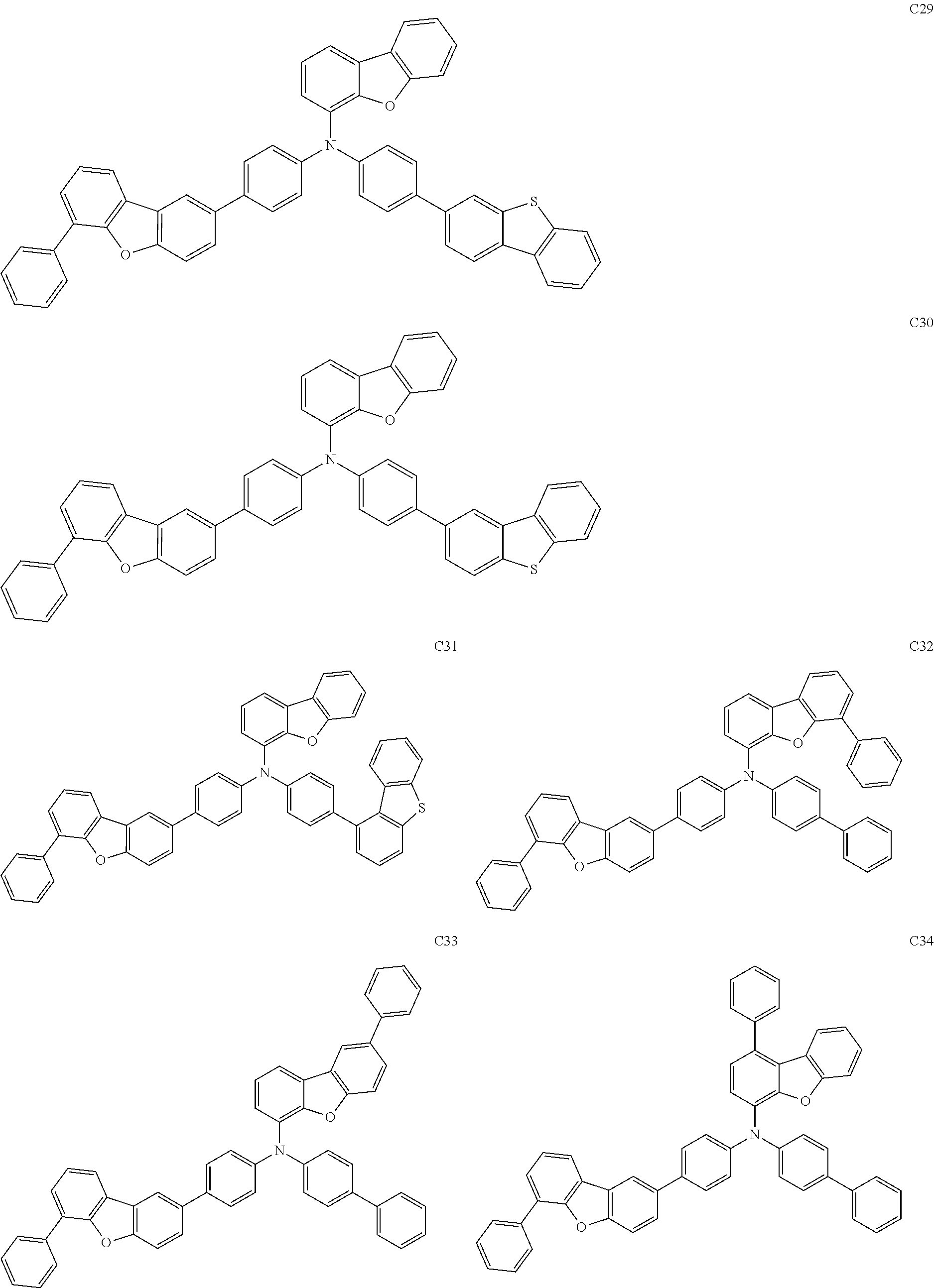 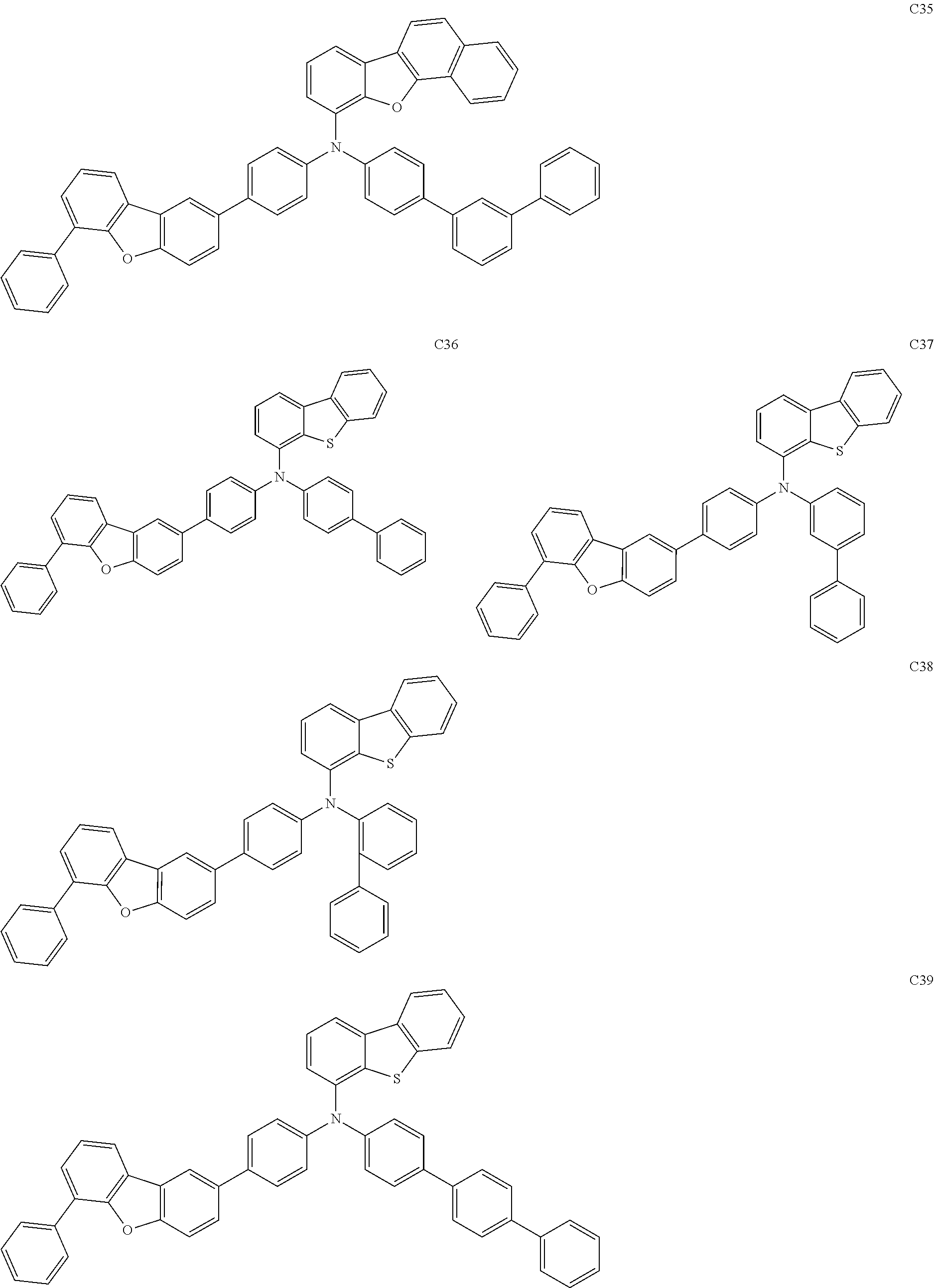 [SMILES: c1ccc(-c2cccc(-c3ccc(N(c4ccc(-c5ccc6oc7c(-c8ccccc8)cccc7c6c5)cc4)c4cccc5c4oc4c6ccccc6ccc54)cc3)c2)cc1 ; c1ccc(-c2ccc(N(c3ccc(-c4ccc5oc6c(-c7ccccc7)cccc6c5c4)cc3)c3cccc4c3sc3ccccc34)cc2)cc1 ; c1ccc(-c2cccc(N(c3ccc(-c4ccc5oc6c(-c7ccccc7)cccc6c5c4)cc3)c3cccc4c3sc3ccccc34)c2)cc1 ; c1ccc(-c2ccccc2N(c2ccc(-c3ccc4oc5c(-c6ccccc6)cccc5c4c3)cc2)c2cccc3c2sc2ccccc23)cc1 ; c1ccc(-c2ccc(-c3ccc(N(c4ccc(-c5ccc6oc7c(-c8ccccc8)cccc7c6c5)cc4)c4cccc5c4sc4ccccc45)cc3)cc2)cc1] 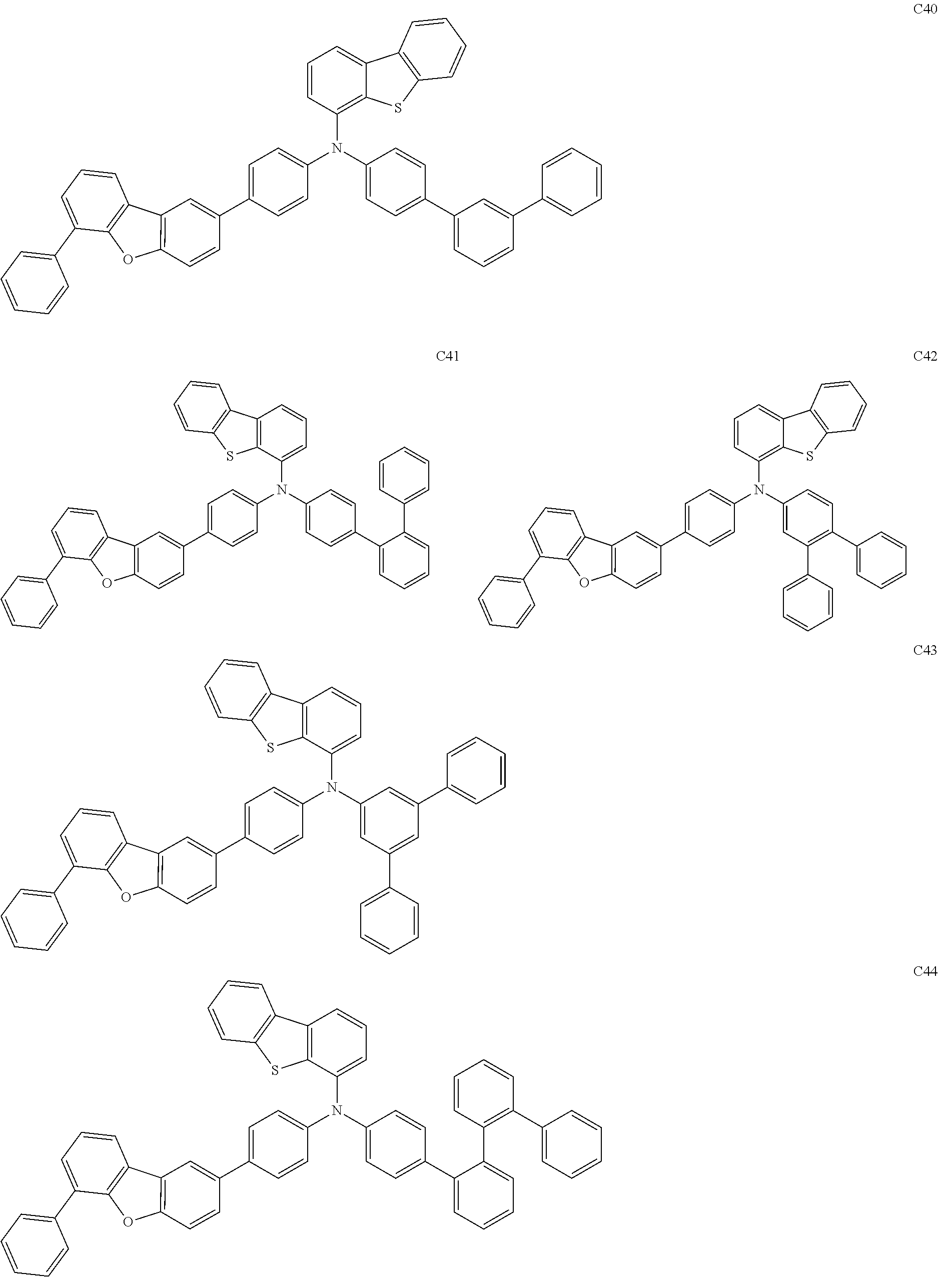 [SMILES: c1ccc(-c2cccc(-c3ccc(N(c4ccc(-c5ccc6oc7c(-c8ccccc8)cccc7c6c5)cc4)c4cccc5c4sc4ccccc45)cc3)c2)cc1 ; c1ccc(-c2ccccc2-c2ccc(N(c3ccc(-c4ccc5oc6c(-c7ccccc7)cccc6c5c4)cc3)c3cccc4c3sc3ccccc34)cc2)cc1 ; c1ccc(-c2ccc(N(c3ccc(-c4ccc5oc6c(-c7ccccc7)cccc6c5c4)cc3)c3cccc4c3sc3ccccc34)cc2-c2ccccc2)cc1 ; c1ccc(-c2cc(-c3ccccc3)cc(N(c3ccc(-c4ccc5oc6c(-c7ccccc7)cccc6c5c4)cc3)c3cccc4c3sc3ccccc34)c2)cc1 ; c1ccc(-c2ccccc2-c2ccccc2-c2ccc(N(c3ccc(-c4ccc5oc6c(-c7ccccc7)cccc6c5c4)cc3)c3cccc4c3sc3ccccc34)cc2)cc1] 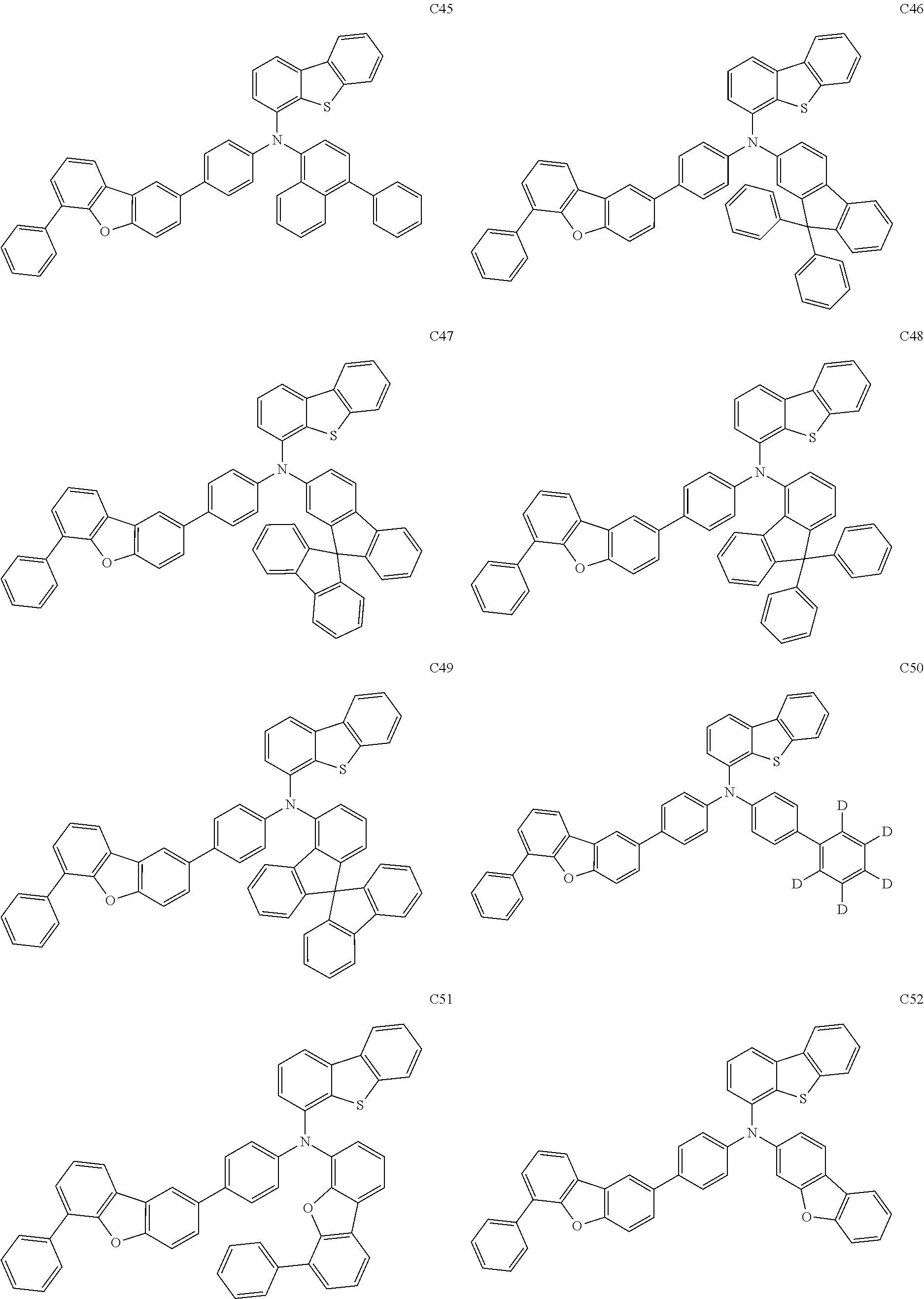 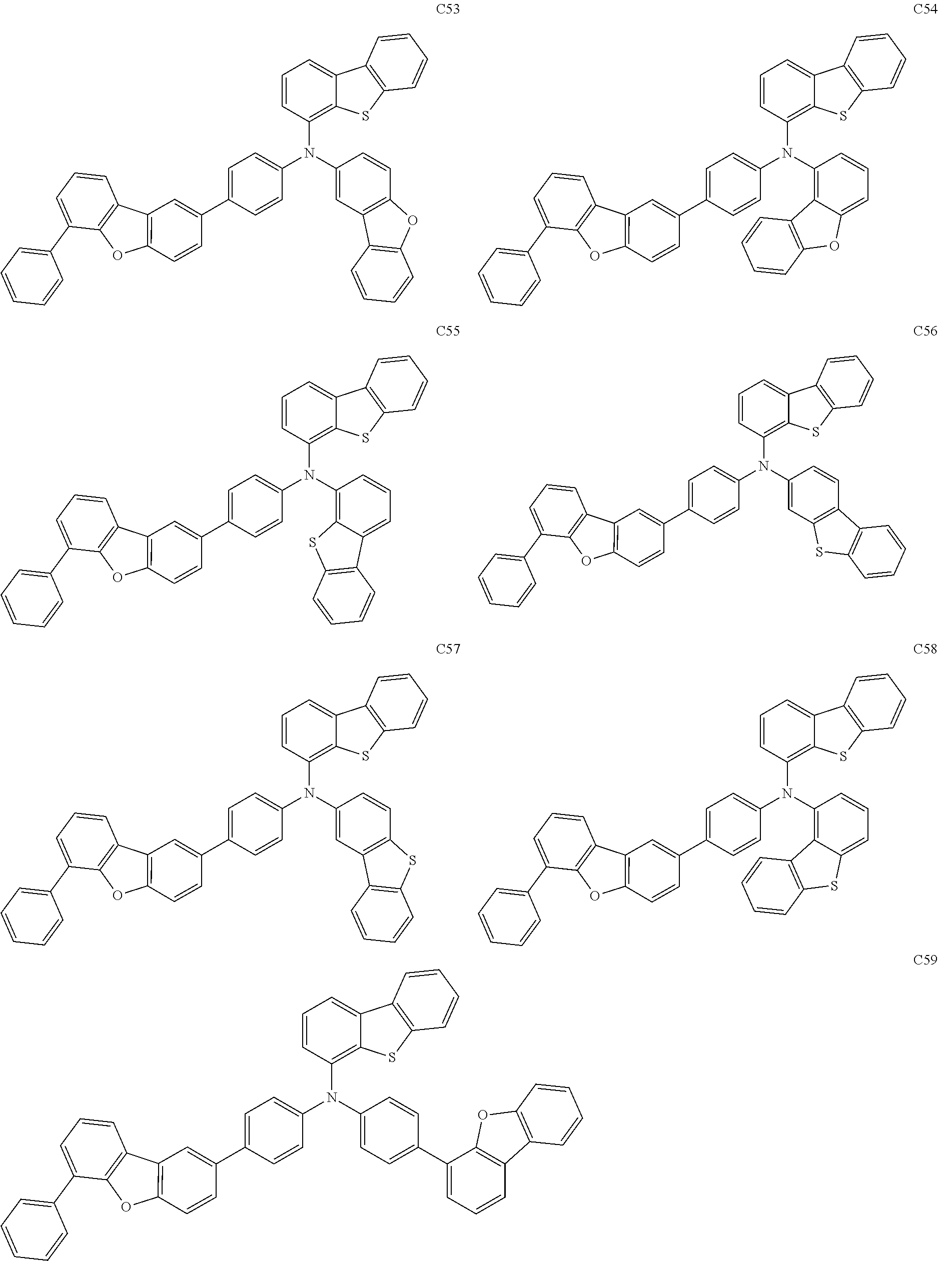 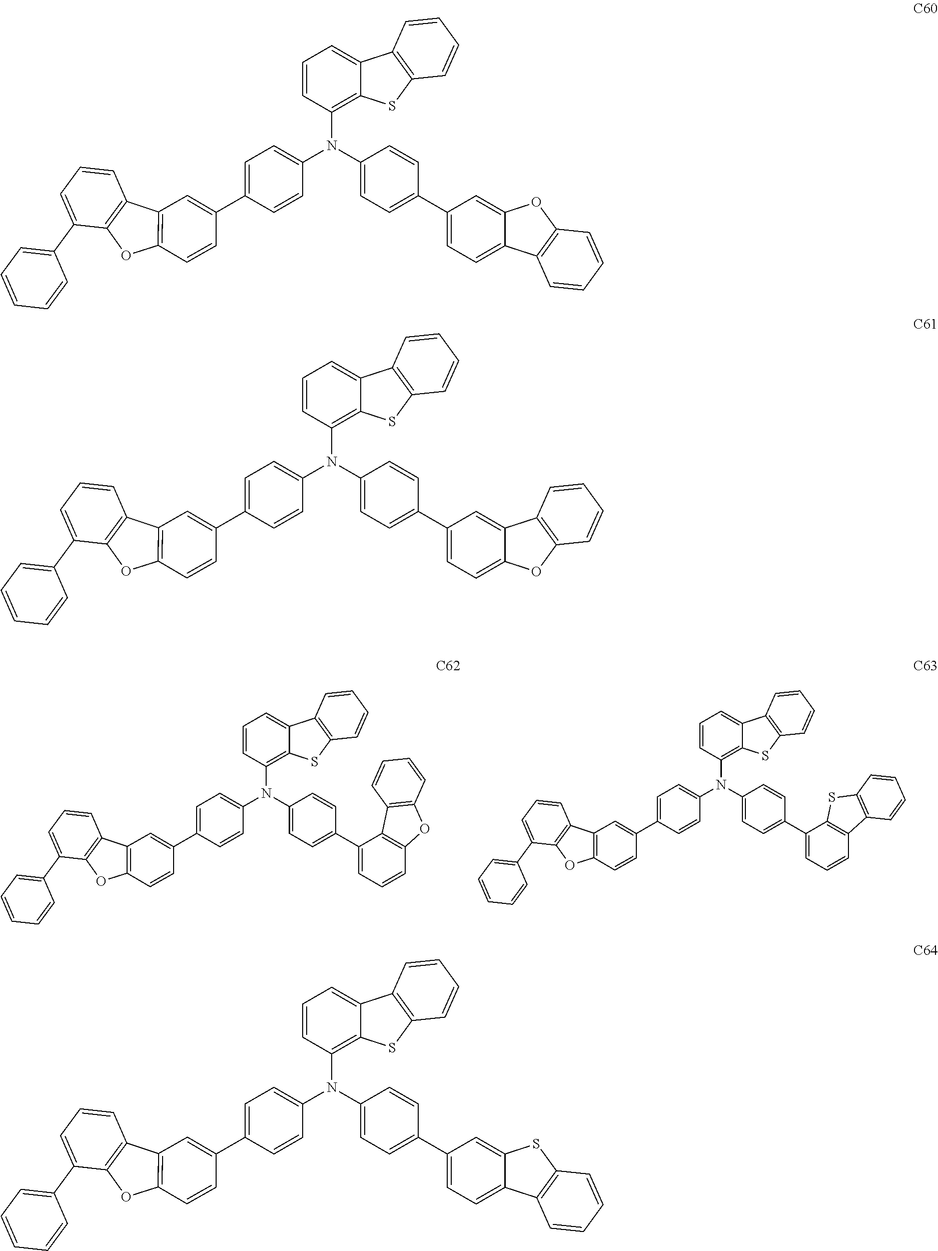 [SMILES: c1ccc(-c2cccc3c2oc2ccc(-c4ccc(N(c5ccc(-c6ccc7c(c6)oc6ccccc67)cc5)c5cccc6c5sc5ccccc56)cc4)cc23)cc1 ; c1ccc(-c2cccc3c2oc2ccc(-c4ccc(N(c5ccc(-c6ccc7oc8ccccc8c7c6)cc5)c5cccc6c5sc5ccccc56)cc4)cc23)cc1 ; c1ccc(-c2cccc3c2oc2ccc(-c4ccc(N(c5ccc(-c6cccc7oc8ccccc8c67)cc5)c5cccc6c5sc5ccccc56)cc4)cc23)cc1 ; c1ccc(-c2cccc3c2oc2ccc(-c4ccc(N(c5ccc(-c6cccc7c6sc6ccccc67)cc5)c5cccc6c5sc5ccccc56)cc4)cc23)cc1 ; c1ccc(-c2cccc3c2oc2ccc(-c4ccc(N(c5ccc(-c6ccc7c(c6)sc6ccccc67)cc5)c5cccc6c5sc5ccccc56)cc4)cc23)cc1] 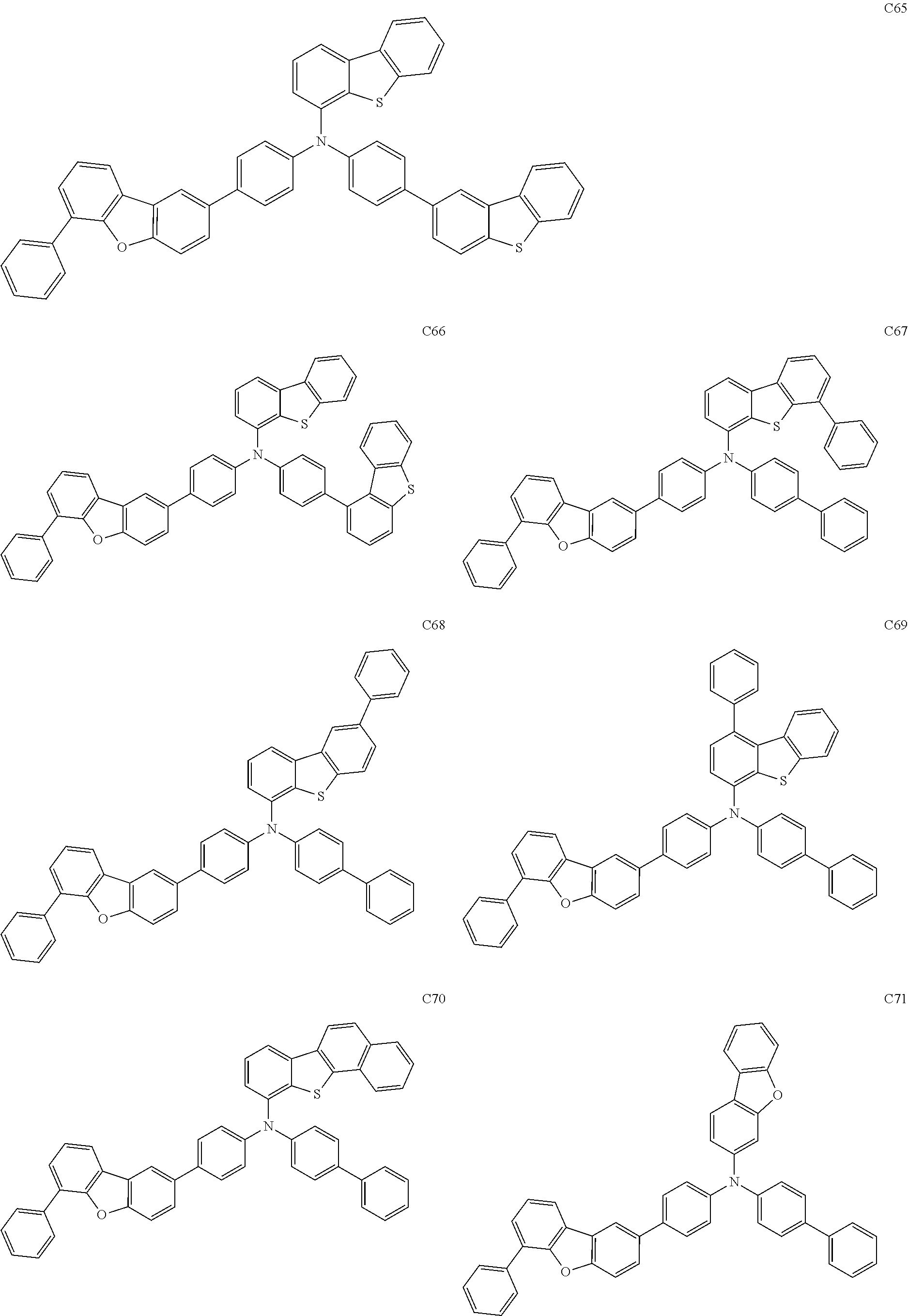 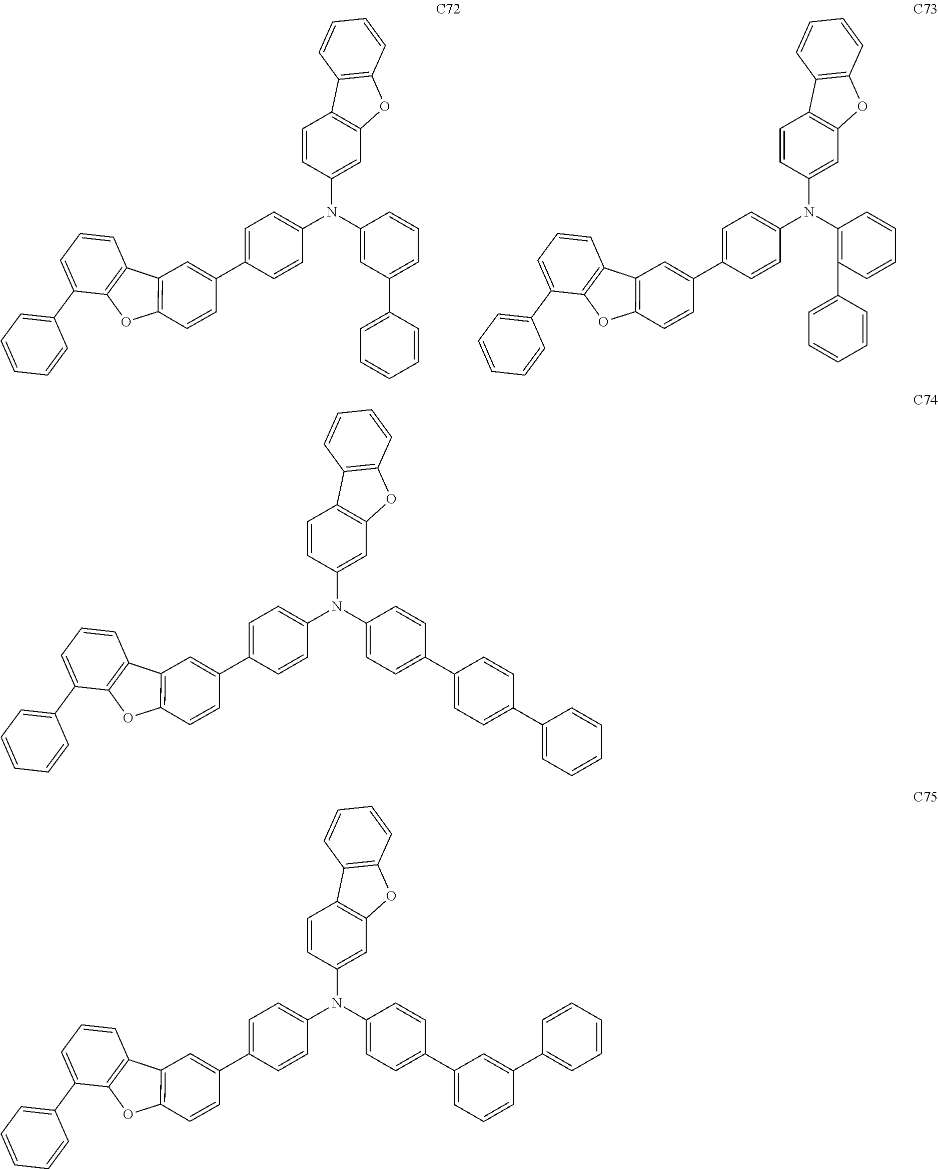 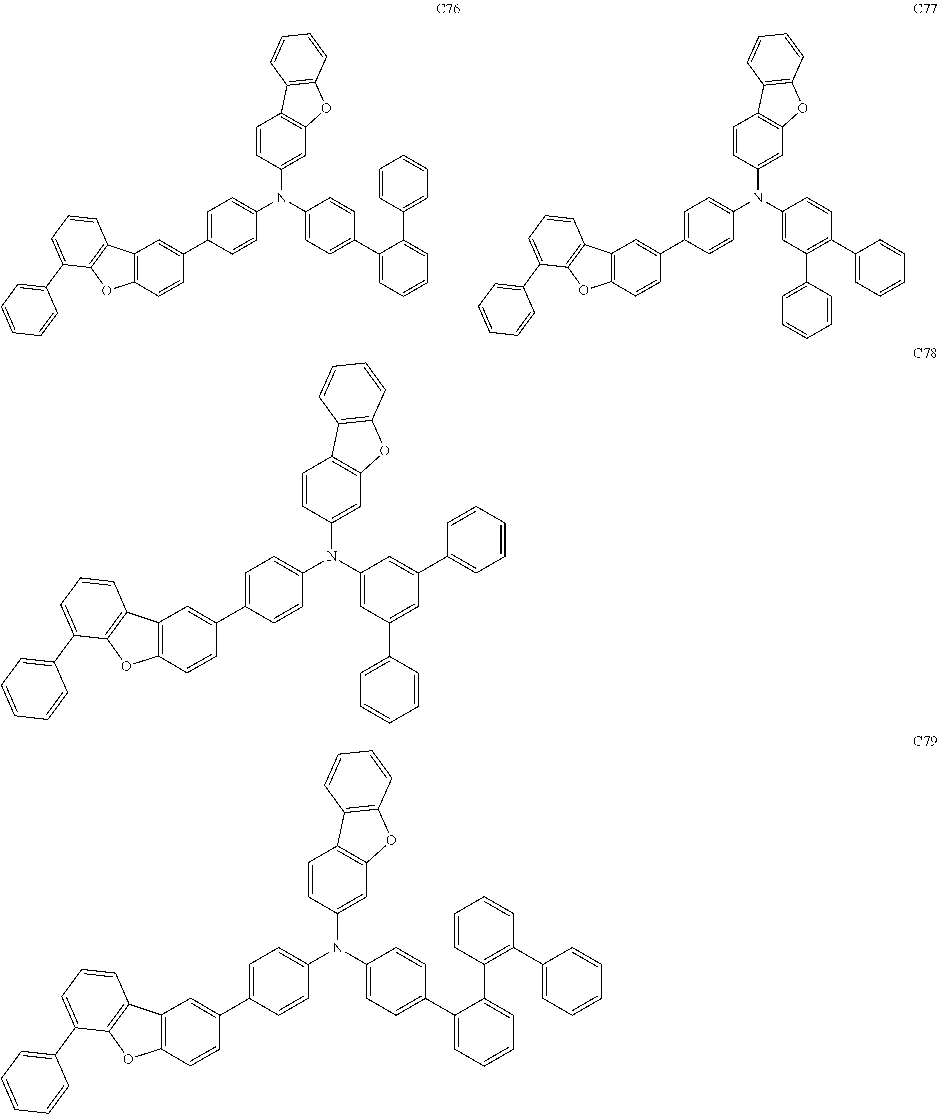 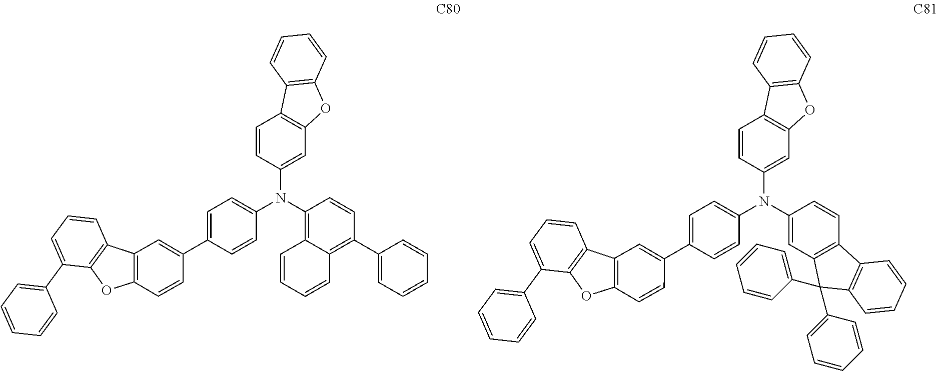 [SMILES: c1ccc(-c2ccc(N(c3ccc(-c4ccc5oc6c(-c7ccccc7)cccc6c5c4)cc3)c3ccc4c(c3)oc3ccccc34)c3ccccc23)cc1 ; c1ccc(-c2cccc3c2oc2ccc(-c4ccc(N(c5ccc6c(c5)C(c5ccccc5)(c5ccccc5)c5ccccc5-6)c5ccc6c(c5)oc5ccccc56)cc4)cc23)cc1] 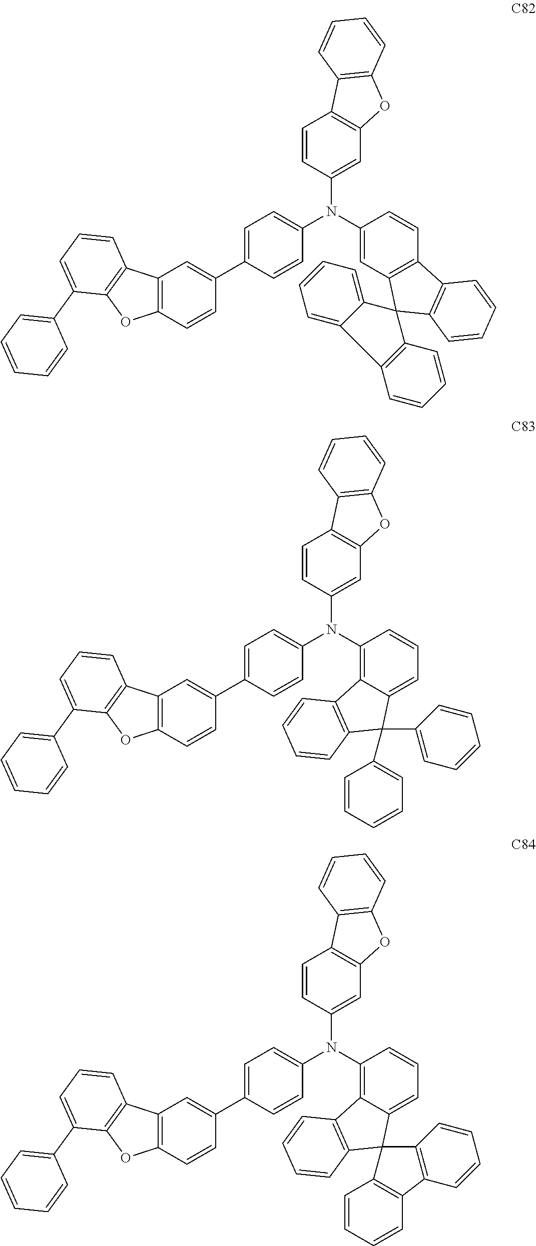 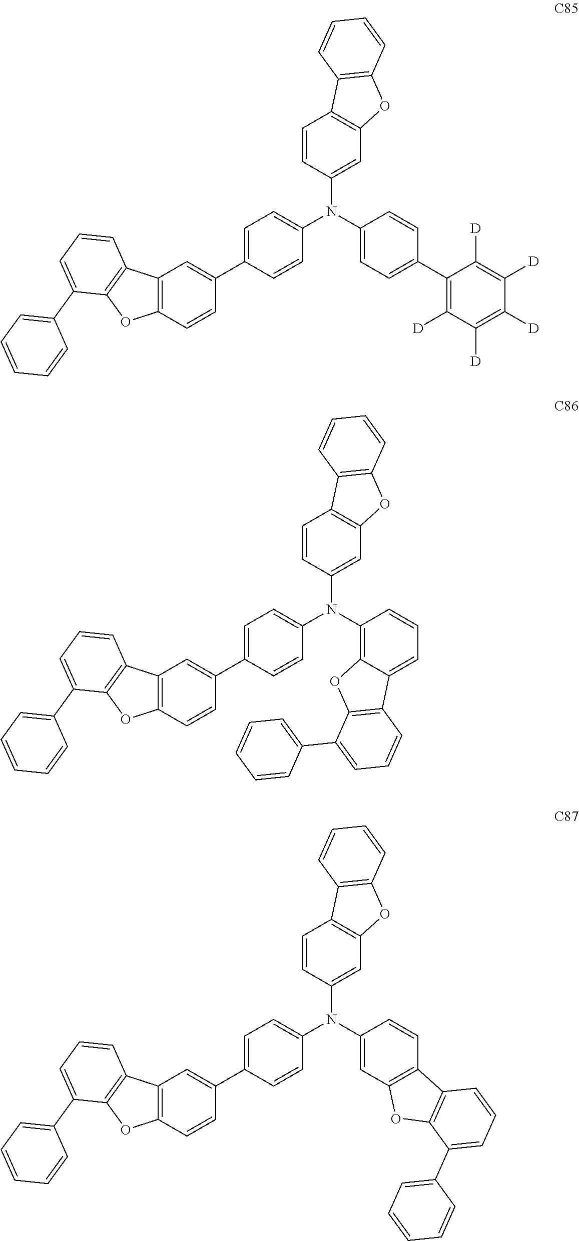 [SMILES: [2H]c1c([2H])c([2H])c(-c2ccc(N(c3ccc(-c4ccc5oc6c(-c7ccccc7)cccc6c5c4)cc3)c3ccc4c(c3)oc3ccccc34)cc2)c([2H])c1[2H] ; c1ccc(-c2cccc3c2oc2ccc(-c4ccc(N(c5ccc6c(c5)oc5ccccc56)c5cccc6c5oc5c(-c7ccccc7)cccc56)cc4)cc23)cc1 ; c1ccc(-c2cccc3c2oc2cc(N(c4ccc(-c5ccc6oc7c(-c8ccccc8)cccc7c6c5)cc4)c4ccc5c(c4)oc4ccccc45)ccc23)cc1] 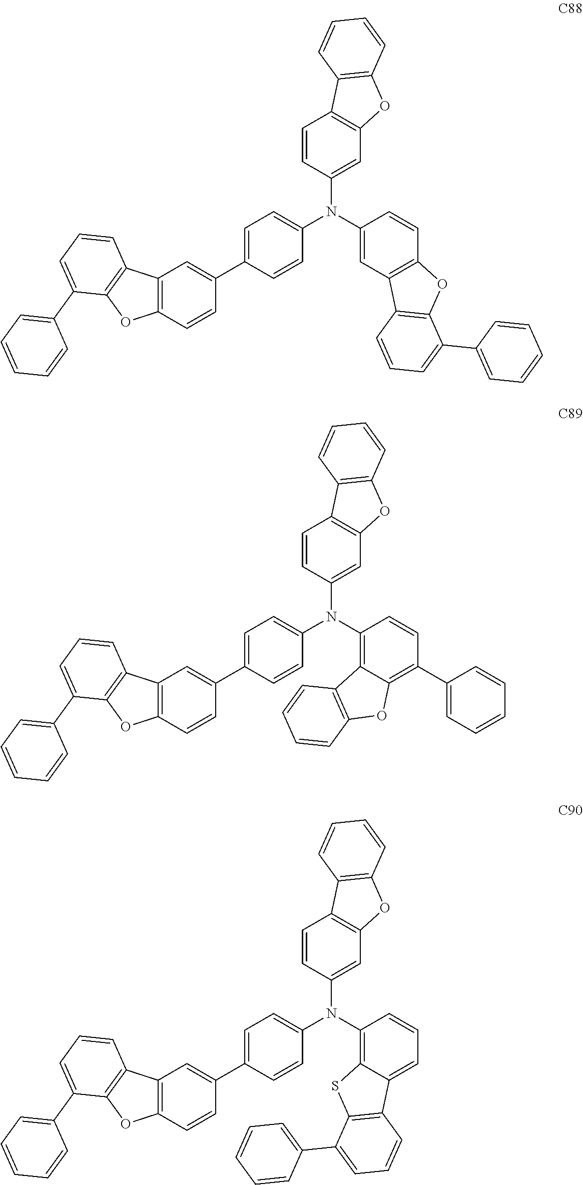 [SMILES: c1ccc(-c2cccc3c2oc2ccc(-c4ccc(N(c5ccc6c(c5)oc5ccccc56)c5ccc6oc7c(-c8ccccc8)cccc7c6c5)cc4)cc23)cc1 ; c1ccc(-c2cccc3c2oc2ccc(-c4ccc(N(c5ccc6c(c5)oc5ccccc56)c5ccc(-c6ccccc6)c6oc7ccccc7c56)cc4)cc23)cc1 ; c1ccc(-c2cccc3c2oc2ccc(-c4ccc(N(c5ccc6c(c5)oc5ccccc56)c5cccc6c5sc5c(-c7ccccc7)cccc56)cc4)cc23)cc1] 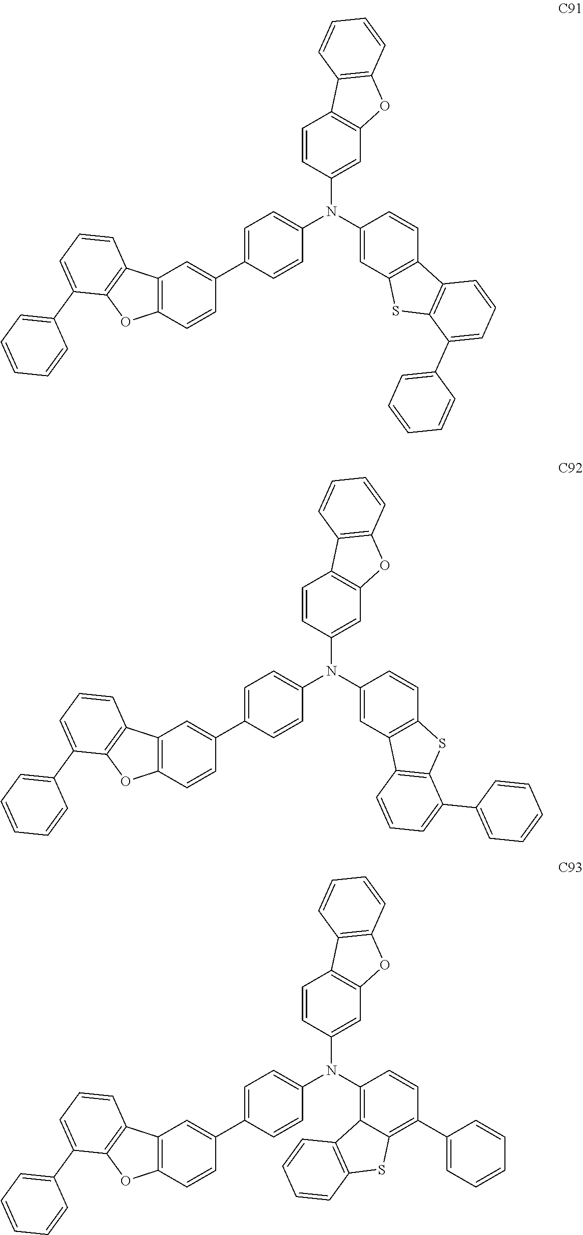 [SMILES: c1ccc(-c2cccc3c2oc2ccc(-c4ccc(N(c5ccc6c(c5)oc5ccccc56)c5ccc6c(c5)sc5c(-c7ccccc7)cccc56)cc4)cc23)cc1 ; c1ccc(-c2cccc3c2oc2ccc(-c4ccc(N(c5ccc6c(c5)oc5ccccc56)c5ccc6sc7c(-c8ccccc8)cccc7c6c5)cc4)cc23)cc1 ; c1ccc(-c2cccc3c2oc2ccc(-c4ccc(N(c5ccc6c(c5)oc5ccccc56)c5ccc(-c6ccccc6)c6sc7ccccc7c56)cc4)cc23)cc1] 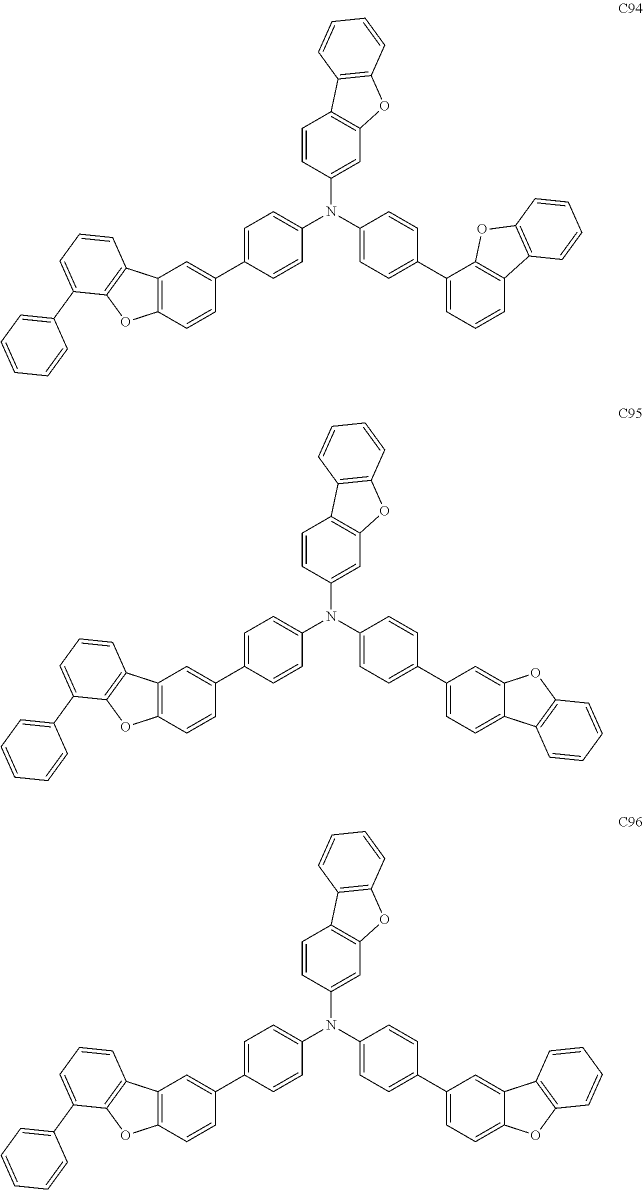 [SMILES: c1ccc(-c2cccc3c2oc2ccc(-c4ccc(N(c5ccc(-c6cccc7c6oc6ccccc67)cc5)c5ccc6c(c5)oc5ccccc56)cc4)cc23)cc1 ; c1ccc(-c2cccc3c2oc2ccc(-c4ccc(N(c5ccc(-c6ccc7c(c6)oc6ccccc67)cc5)c5ccc6c(c5)oc5ccccc56)cc4)cc23)cc1 ; c1ccc(-c2cccc3c2oc2ccc(-c4ccc(N(c5ccc(-c6ccc7oc8ccccc8c7c6)cc5)c5ccc6c(c5)oc5ccccc56)cc4)cc23)cc1] 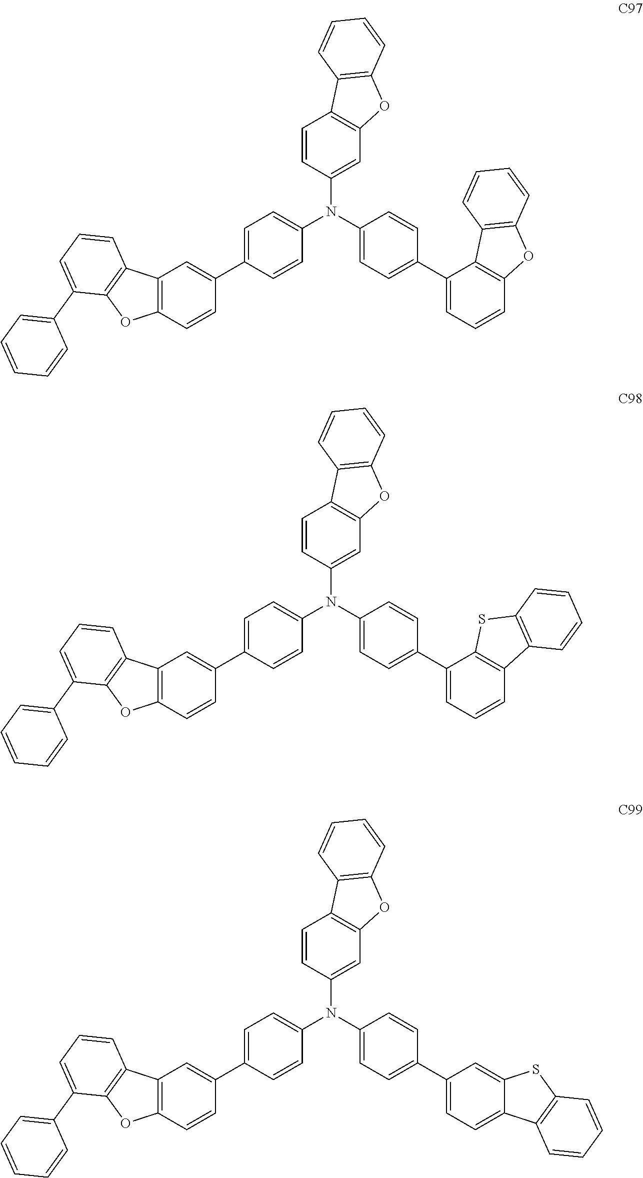 [SMILES: c1ccc(-c2cccc3c2oc2ccc(-c4ccc(N(c5ccc(-c6cccc7oc8ccccc8c67)cc5)c5ccc6c(c5)oc5ccccc56)cc4)cc23)cc1 ; c1ccc(-c2cccc3c2oc2ccc(-c4ccc(N(c5ccc(-c6cccc7c6sc6ccccc67)cc5)c5ccc6c(c5)oc5ccccc56)cc4)cc23)cc1 ; c1ccc(-c2cccc3c2oc2ccc(-c4ccc(N(c5ccc(-c6ccc7c(c6)sc6ccccc67)cc5)c5ccc6c(c5)oc5ccccc56)cc4)cc23)cc1] 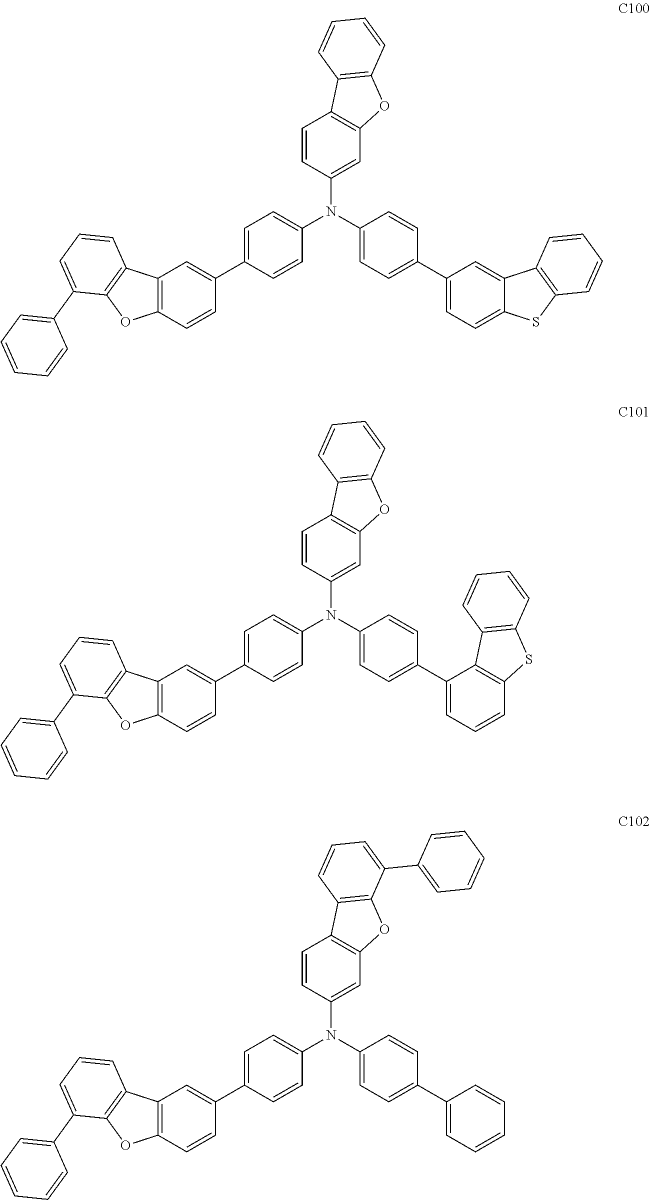 [SMILES: c1ccc(-c2cccc3c2oc2ccc(-c4ccc(N(c5ccc(-c6ccc7sc8ccccc8c7c6)cc5)c5ccc6c(c5)oc5ccccc56)cc4)cc23)cc1 ; c1ccc(-c2cccc3c2oc2ccc(-c4ccc(N(c5ccc(-c6cccc7sc8ccccc8c67)cc5)c5ccc6c(c5)oc5ccccc56)cc4)cc23)cc1 ; c1ccc(-c2ccc(N(c3ccc(-c4ccc5oc6c(-c7ccccc7)cccc6c5c4)cc3)c3ccc4c(c3)oc3c(-c5ccccc5)cccc34)cc2)cc1] 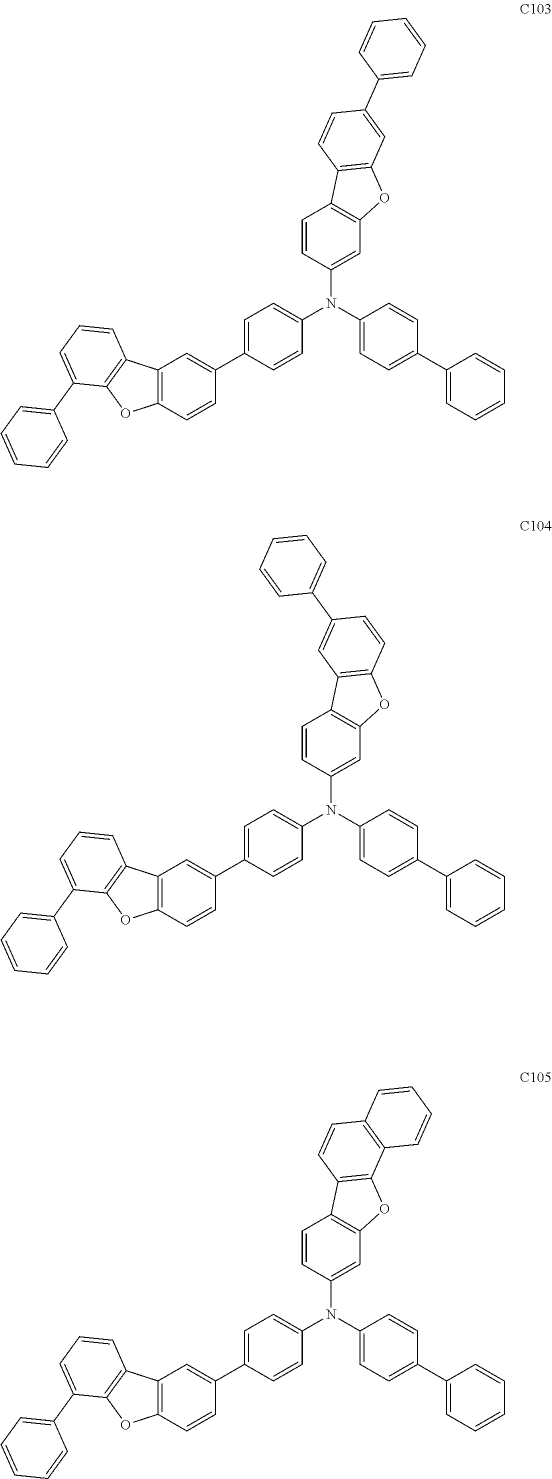 [SMILES: c1ccc(-c2ccc(N(c3ccc(-c4ccc5oc6c(-c7ccccc7)cccc6c5c4)cc3)c3ccc4c(c3)oc3cc(-c5ccccc5)ccc34)cc2)cc1 ; c1ccc(-c2ccc(N(c3ccc(-c4ccc5oc6c(-c7ccccc7)cccc6c5c4)cc3)c3ccc4c(c3)oc3ccc(-c5ccccc5)cc34)cc2)cc1 ; c1ccc(-c2ccc(N(c3ccc(-c4ccc5oc6c(-c7ccccc7)cccc6c5c4)cc3)c3ccc4c(c3)oc3c5ccccc5ccc43)cc2)cc1] 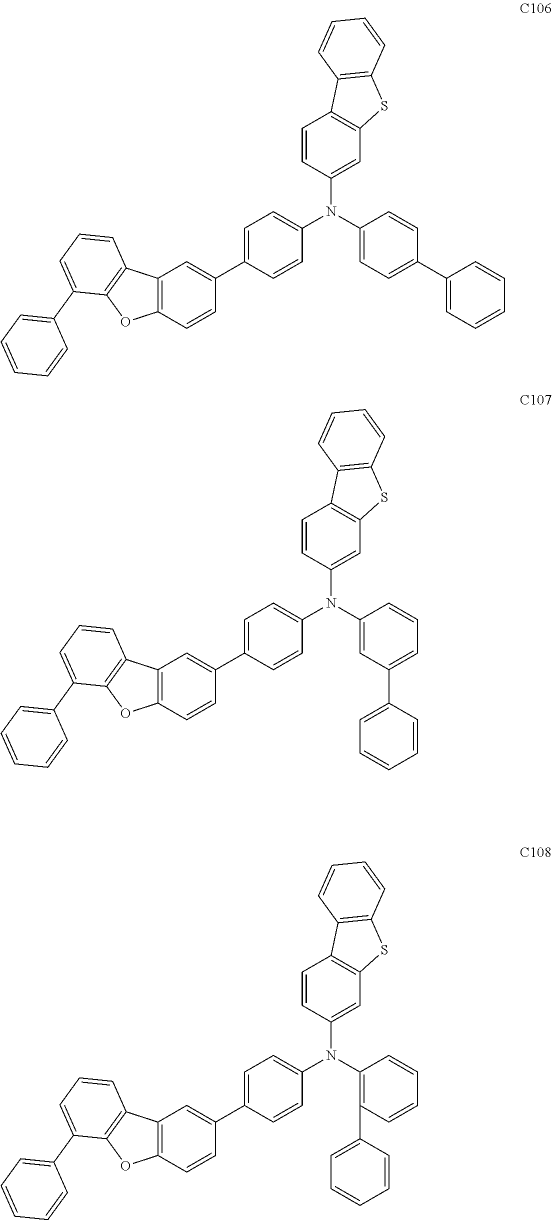 [SMILES: c1ccc(-c2ccc(N(c3ccc(-c4ccc5oc6c(-c7ccccc7)cccc6c5c4)cc3)c3ccc4c(c3)sc3ccccc34)cc2)cc1 ; c1ccc(-c2cccc(N(c3ccc(-c4ccc5oc6c(-c7ccccc7)cccc6c5c4)cc3)c3ccc4c(c3)sc3ccccc34)c2)cc1 ; c1ccc(-c2ccccc2N(c2ccc(-c3ccc4oc5c(-c6ccccc6)cccc5c4c3)cc2)c2ccc3c(c2)sc2ccccc23)cc1] 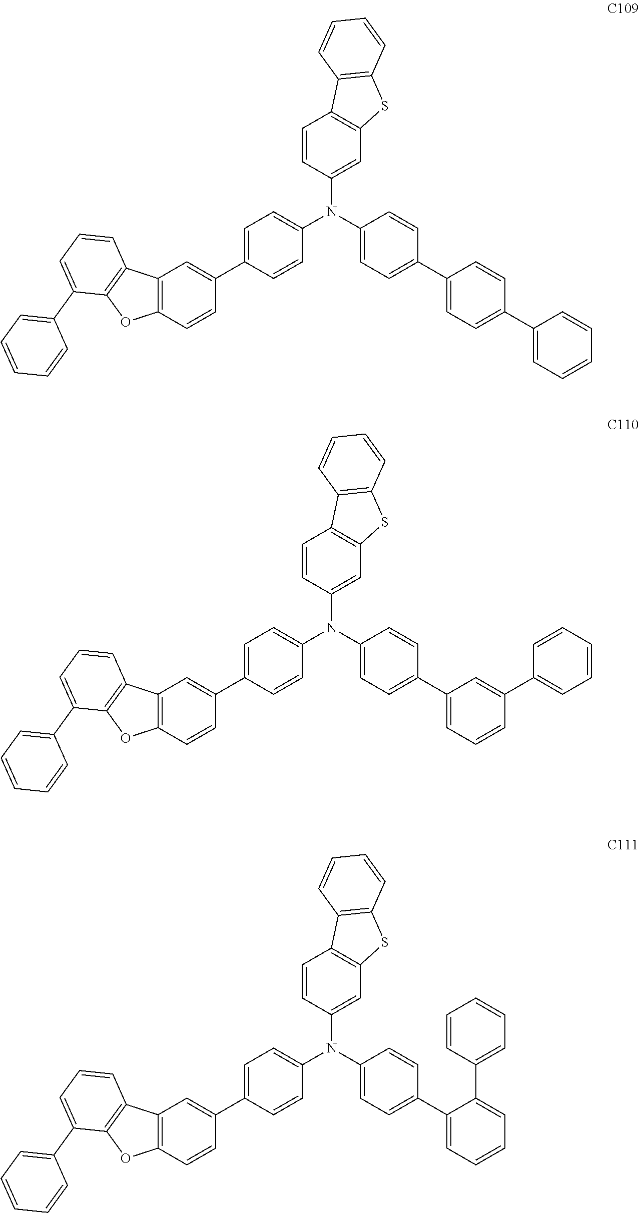 [SMILES: c1ccc(-c2ccc(-c3ccc(N(c4ccc(-c5ccc6oc7c(-c8ccccc8)cccc7c6c5)cc4)c4ccc5c(c4)sc4ccccc45)cc3)cc2)cc1 ; c1ccc(-c2cccc(-c3ccc(N(c4ccc(-c5ccc6oc7c(-c8ccccc8)cccc7c6c5)cc4)c4ccc5c(c4)sc4ccccc45)cc3)c2)cc1 ; c1ccc(-c2ccccc2-c2ccc(N(c3ccc(-c4ccc5oc6c(-c7ccccc7)cccc6c5c4)cc3)c3ccc4c(c3)sc3ccccc34)cc2)cc1] 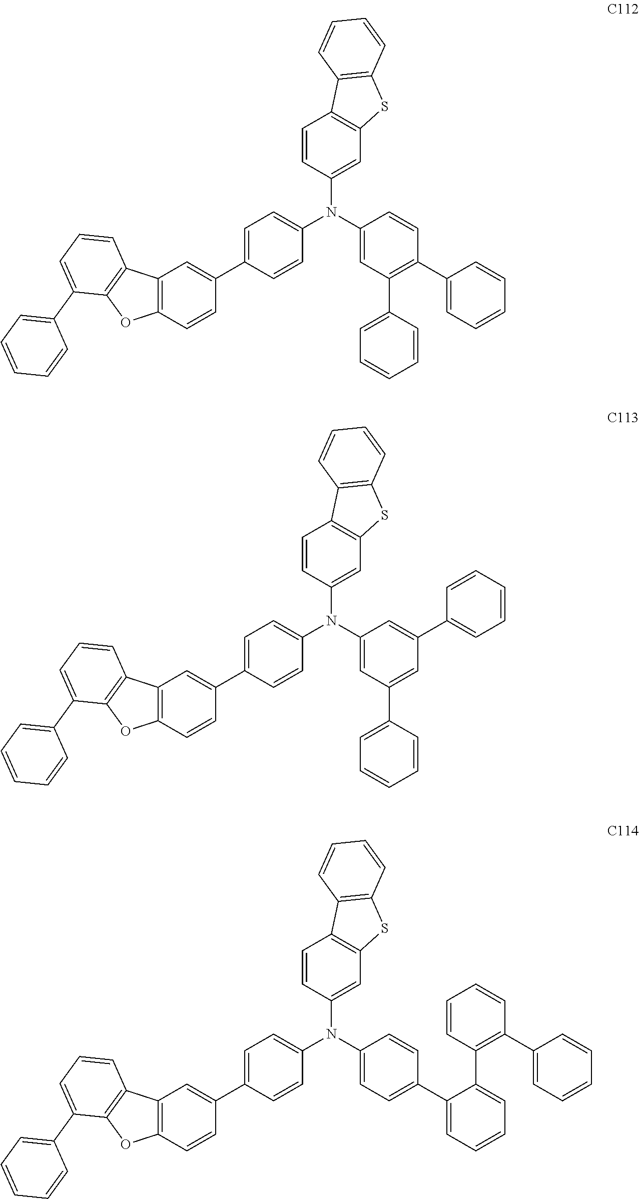 [SMILES: c1ccc(-c2ccc(N(c3ccc(-c4ccc5oc6c(-c7ccccc7)cccc6c5c4)cc3)c3ccc4c(c3)sc3ccccc34)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc4c(c3)sc3ccccc34)c2)cc1 ; c1ccc(-c2ccccc2-c2ccccc2-c2ccc(N(c3ccc(-c4ccc5oc6c(-c7ccccc7)cccc6c5c4)cc3)c3ccc4c(c3)sc3ccccc34)cc2)cc1] 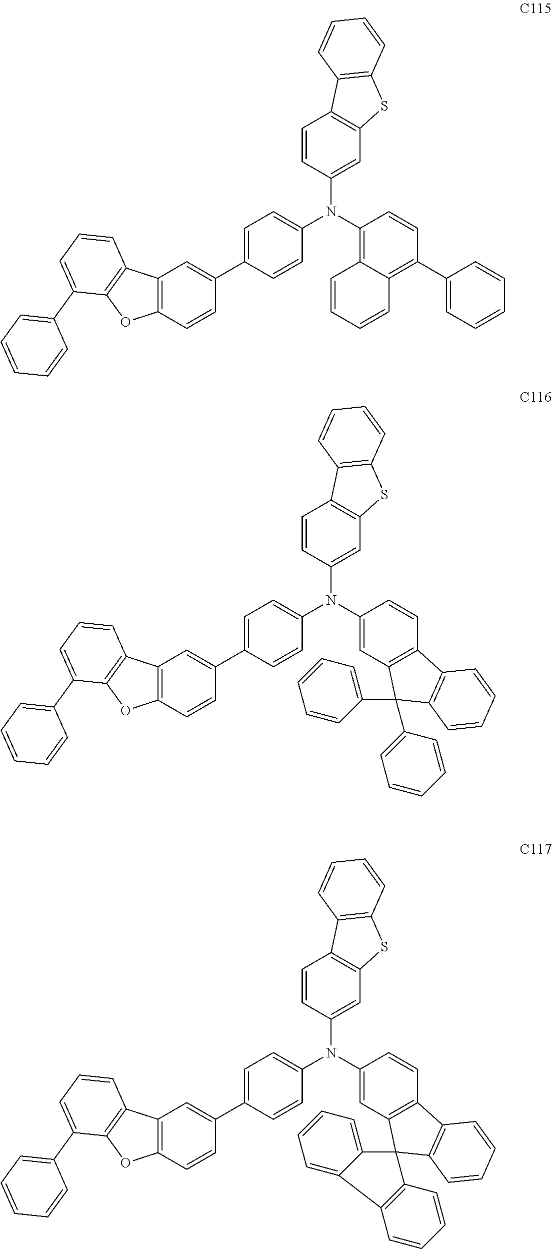 [SMILES: c1ccc(-c2ccc(N(c3ccc(-c4ccc5oc6c(-c7ccccc7)cccc6c5c4)cc3)c3ccc4c(c3)sc3ccccc34)c3ccccc23)cc1 ; c1ccc(-c2cccc3c2oc2ccc(-c4ccc(N(c5ccc6c(c5)C(c5ccccc5)(c5ccccc5)c5ccccc5-6)c5ccc6c(c5)sc5ccccc56)cc4)cc23)cc1 ; c1ccc(-c2cccc3c2oc2ccc(-c4ccc(N(c5ccc6c(c5)C5(c7ccccc7-c7ccccc75)c5ccccc5-6)c5ccc6c(c5)sc5ccccc56)cc4)cc23)cc1] 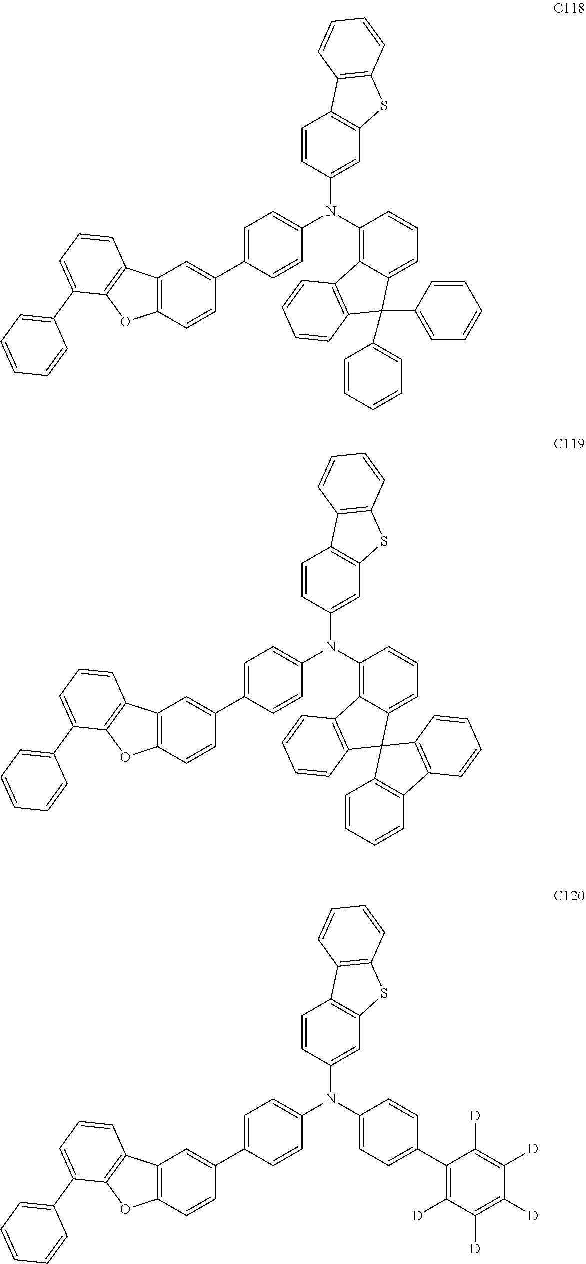 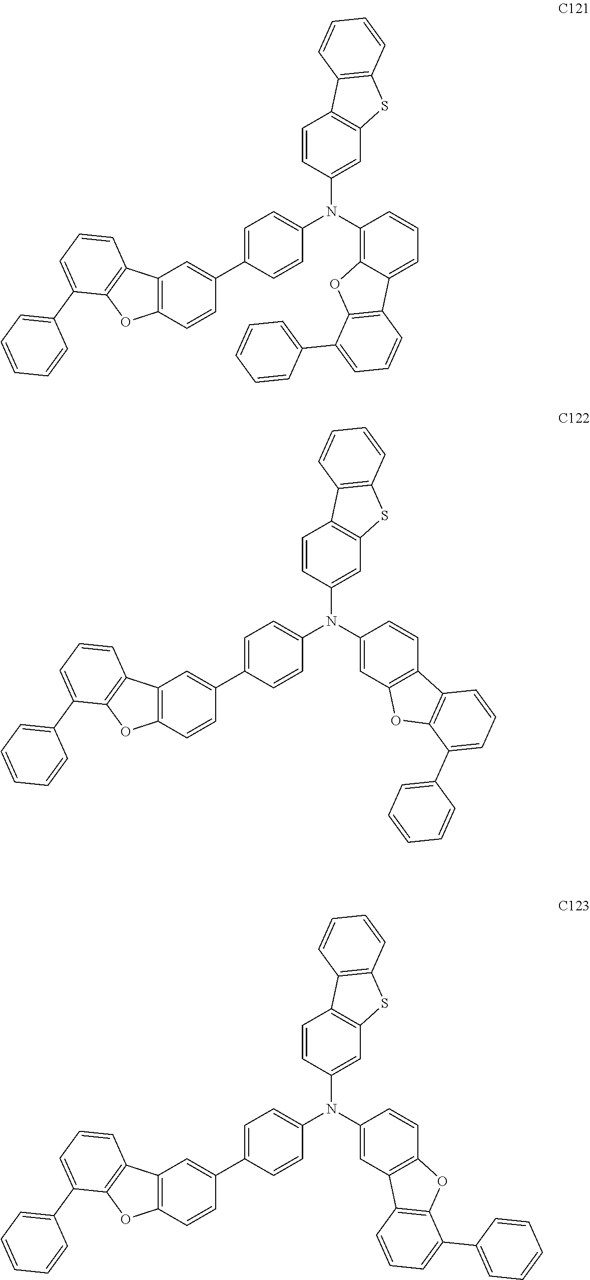 [SMILES: c1ccc(-c2cccc3c2oc2ccc(-c4ccc(N(c5ccc6c(c5)sc5ccccc56)c5cccc6c5oc5c(-c7ccccc7)cccc56)cc4)cc23)cc1 ; c1ccc(-c2cccc3c2oc2cc(N(c4ccc(-c5ccc6oc7c(-c8ccccc8)cccc7c6c5)cc4)c4ccc5c(c4)sc4ccccc45)ccc23)cc1 ; c1ccc(-c2cccc3c2oc2ccc(-c4ccc(N(c5ccc6c(c5)sc5ccccc56)c5ccc6oc7c(-c8ccccc8)cccc7c6c5)cc4)cc23)cc1] 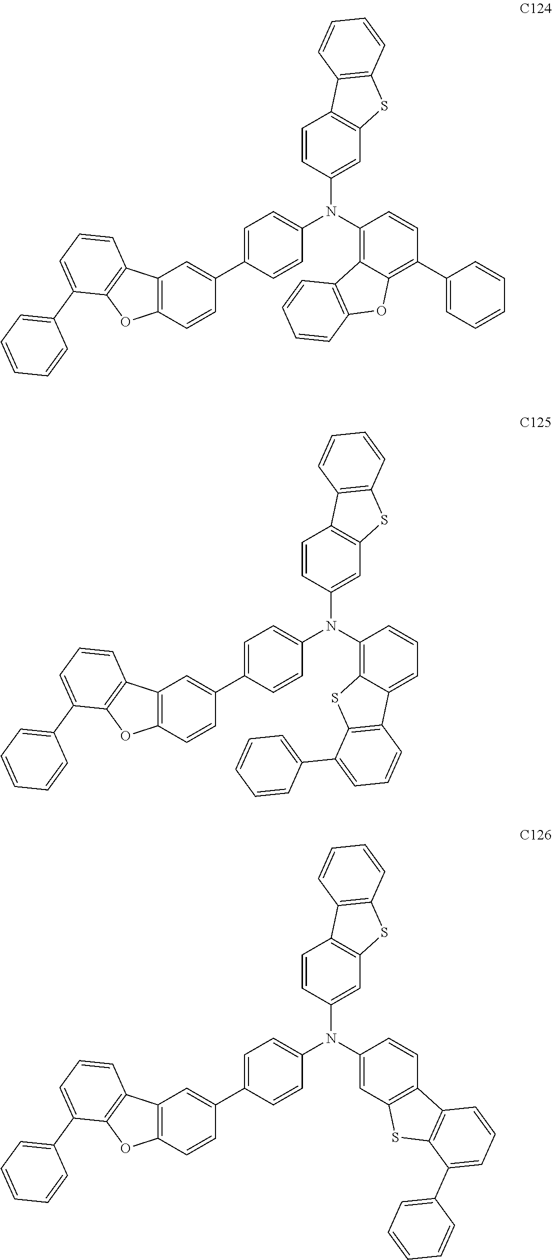 [SMILES: c1ccc(-c2cccc3c2oc2ccc(-c4ccc(N(c5ccc6c(c5)sc5ccccc56)c5ccc(-c6ccccc6)c6oc7ccccc7c56)cc4)cc23)cc1 ; c1ccc(-c2cccc3c2oc2ccc(-c4ccc(N(c5ccc6c(c5)sc5ccccc56)c5cccc6c5sc5c(-c7ccccc7)cccc56)cc4)cc23)cc1 ; c1ccc(-c2cccc3c2oc2ccc(-c4ccc(N(c5ccc6c(c5)sc5ccccc56)c5ccc6c(c5)sc5c(-c7ccccc7)cccc56)cc4)cc23)cc1] 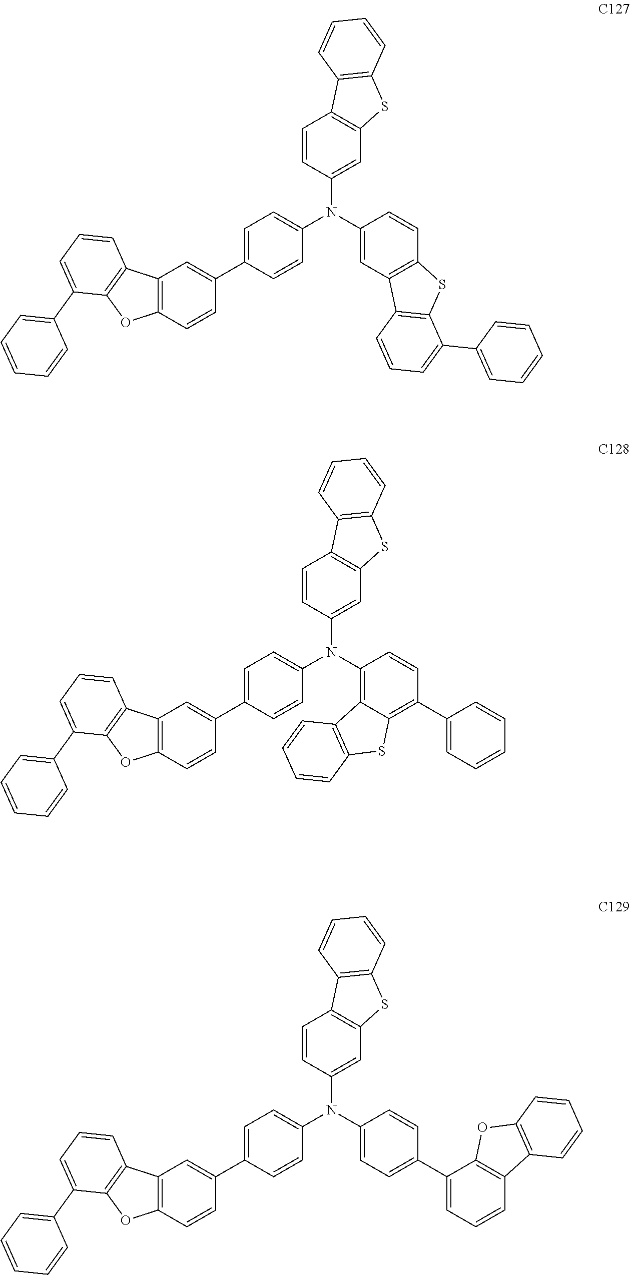 [SMILES: c1ccc(-c2cccc3c2oc2ccc(-c4ccc(N(c5ccc6c(c5)sc5ccccc56)c5ccc6sc7c(-c8ccccc8)cccc7c6c5)cc4)cc23)cc1 ; c1ccc(-c2cccc3c2oc2ccc(-c4ccc(N(c5ccc6c(c5)sc5ccccc56)c5ccc(-c6ccccc6)c6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc6c(c5)sc5ccccc56)cc4)cc23)cc1] 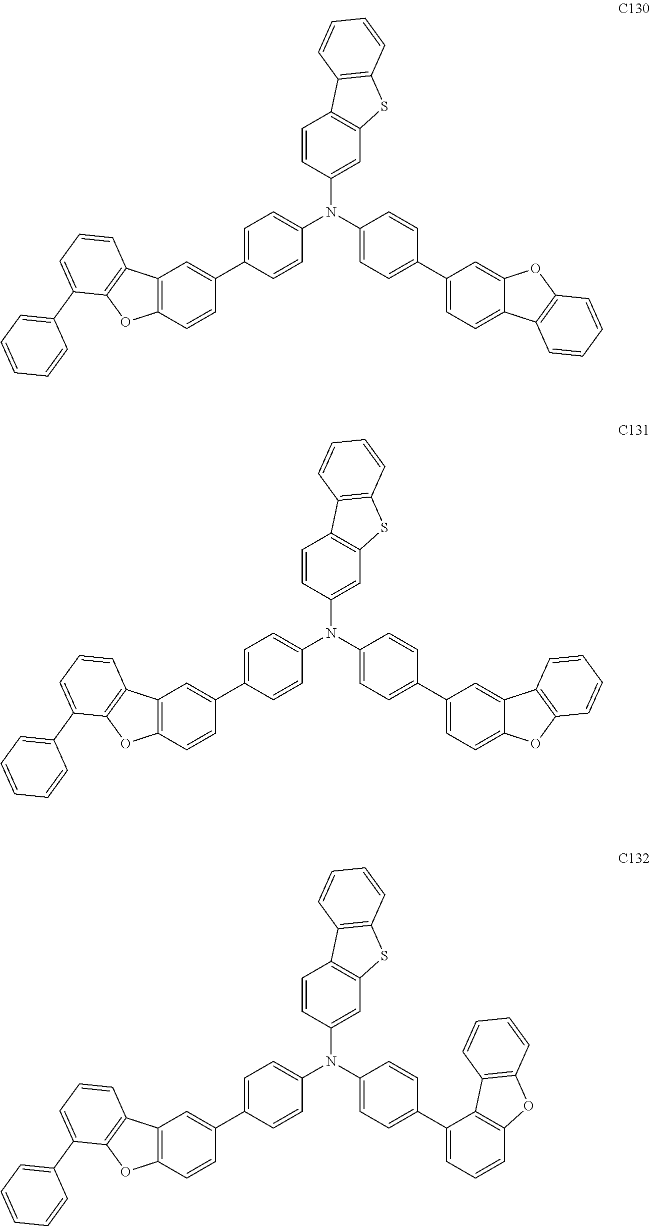 [SMILES: c1ccc(-c2cccc3c2oc2ccc(-c4ccc(N(c5ccc(-c6ccc7c(c6)oc6ccccc67)cc5)c5ccc6c(c5)sc5ccccc56)cc4)cc23)cc1 ; c1ccc(-c2cccc3c2oc2ccc(-c4ccc(N(c5ccc(-c6ccc7oc8ccccc8c7c6)cc5)c5ccc6c(c5)sc5ccccc56)cc4)cc23)cc1 ; c1ccc(-c2cccc3c2oc2ccc(-c4ccc(N(c5ccc(-c6cccc7oc8ccccc8c67)cc5)c5ccc6c(c5)sc5ccccc56)cc4)cc23)cc1] 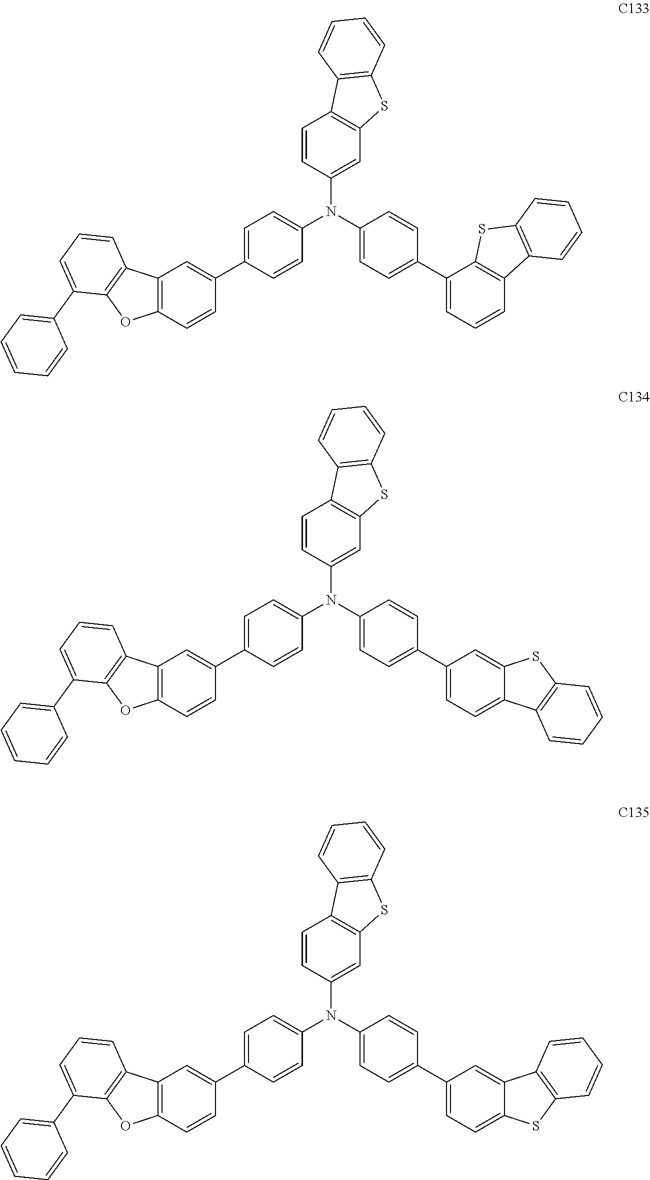 [SMILES: c1ccc(-c2cccc3c2oc2ccc(-c4ccc(N(c5ccc(-c6cccc7c6sc6ccccc67)cc5)c5ccc6c(c5)sc5ccccc56)cc4)cc23)cc1 ; c1ccc(-c2cccc3c2oc2ccc(-c4ccc(N(c5ccc(-c6ccc7c(c6)sc6ccccc67)cc5)c5ccc6c(c5)sc5ccccc56)cc4)cc23)cc1 ; c1ccc(-c2cccc3c2oc2ccc(-c4ccc(N(c5ccc(-c6ccc7sc8ccccc8c7c6)cc5)c5ccc6c(c5)sc5ccccc56)cc4)cc23)cc1] 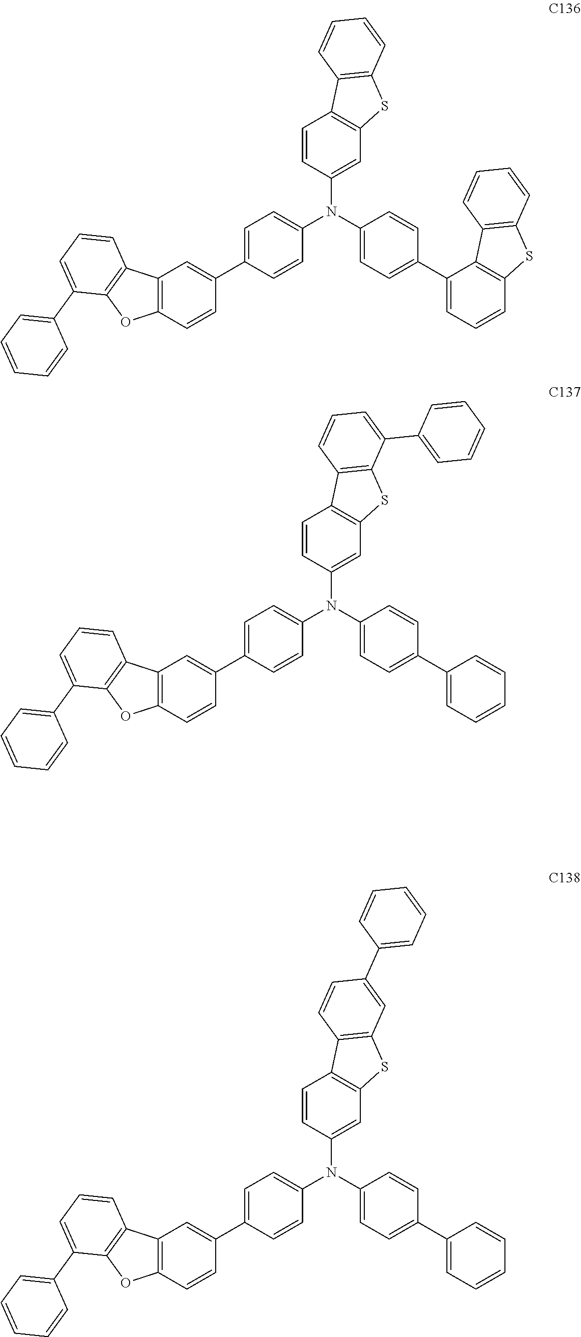 [SMILES: c1ccc(-c2cccc3c2oc2ccc(-c4ccc(N(c5ccc(-c6cccc7sc8ccccc8c67)cc5)c5ccc6c(c5)sc5ccccc56)cc4)cc23)cc1 ; c1ccc(-c2ccc(N(c3ccc(-c4ccc5oc6c(-c7ccccc7)cccc6c5c4)cc3)c3ccc4c(c3)sc3c(-c5ccccc5)cccc34)cc2)cc1 ; c1ccc(-c2ccc(N(c3ccc(-c4ccc5oc6c(-c7ccccc7)cccc6c5c4)cc3)c3ccc4c(c3)sc3cc(-c5ccccc5)ccc34)cc2)cc1] 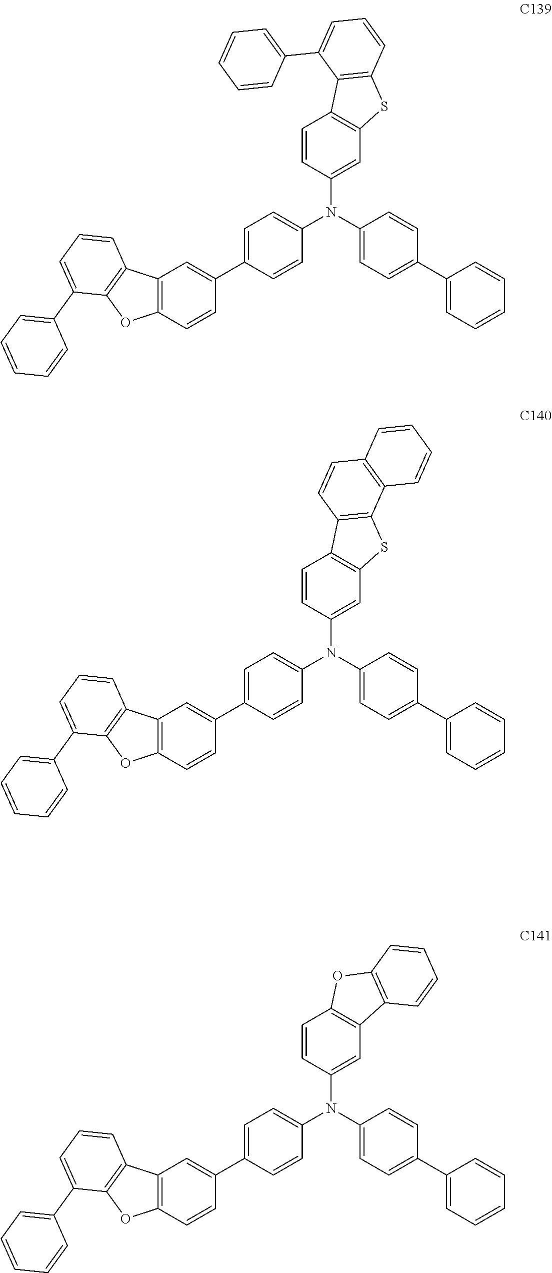 [SMILES: c1ccc(-c2ccc(N(c3ccc(-c4ccc5oc6c(-c7ccccc7)cccc6c5c4)cc3)c3ccc4c(c3)sc3cccc(-c5ccccc5)c34)cc2)cc1 ; c1ccc(-c2ccc(N(c3ccc(-c4ccc5oc6c(-c7ccccc7)cccc6c5c4)cc3)c3ccc4c(c3)sc3c5ccccc5ccc43)cc2)cc1 ; c1ccc(-c2ccc(N(c3ccc(-c4ccc5oc6c(-c7ccccc7)cccc6c5c4)cc3)c3ccc4oc5ccccc5c4c3)cc2)cc1] 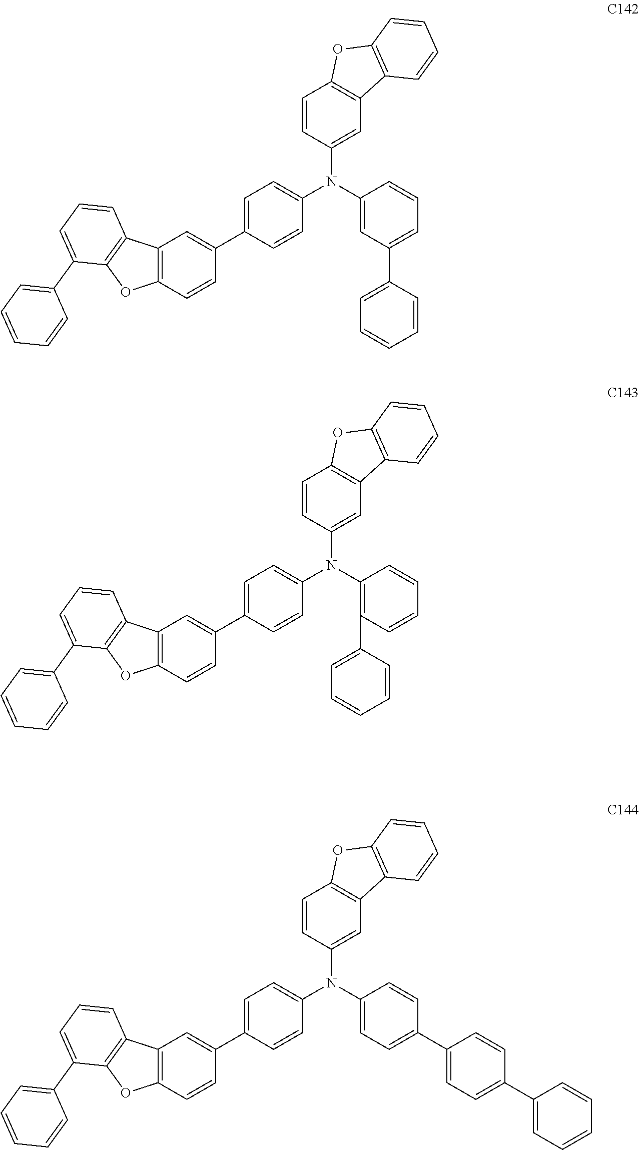 [SMILES: c1ccc(-c2cccc(N(c3ccc(-c4ccc5oc6c(-c7ccccc7)cccc6c5c4)cc3)c3ccc4oc5ccccc5c4c3)c2)cc1 ; c1ccc(-c2ccccc2N(c2ccc(-c3ccc4oc5c(-c6ccccc6)cccc5c4c3)cc2)c2ccc3oc4ccccc4c3c2)cc1 ; c1ccc(-c2ccc(-c3ccc(N(c4ccc(-c5ccc6oc7c(-c8ccccc8)cccc7c6c5)cc4)c4ccc5oc6ccccc6c5c4)cc3)cc2)cc1] 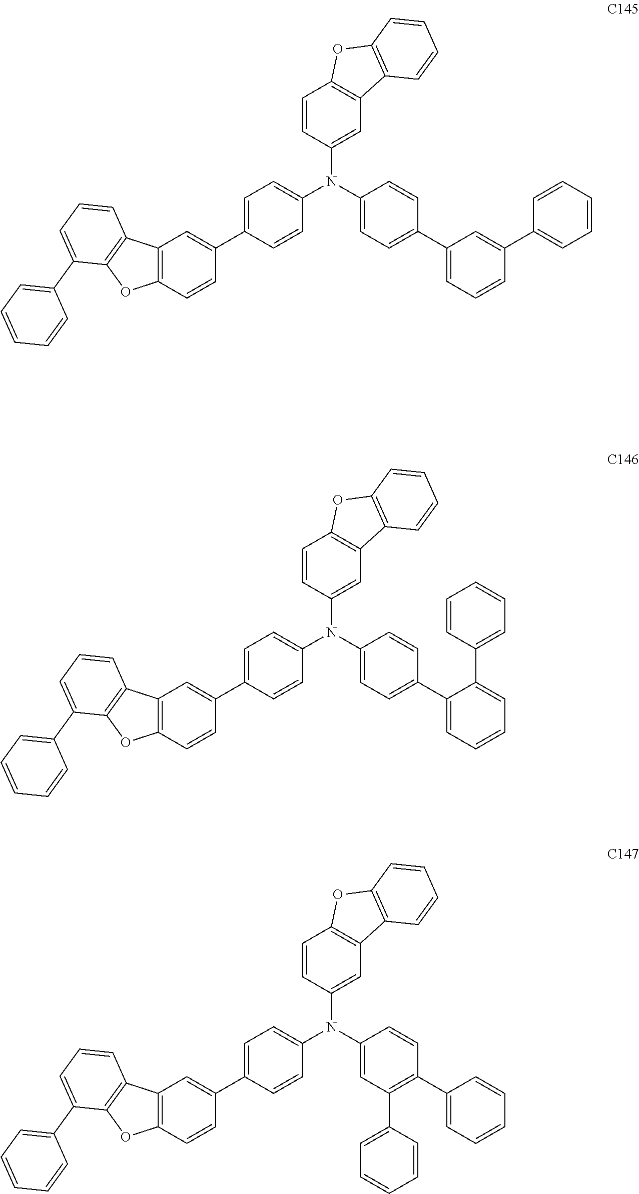 [SMILES: c1ccc(-c2cccc(-c3ccc(N(c4ccc(-c5ccc6oc7c(-c8ccccc8)cccc7c6c5)cc4)c4ccc5oc6ccccc6c5c4)cc3)c2)cc1 ; c1ccc(-c2ccccc2-c2ccc(N(c3ccc(-c4ccc5oc6c(-c7ccccc7)cccc6c5c4)cc3)c3ccc4oc5ccccc5c4c3)cc2)cc1 ; c1ccc(-c2ccc(N(c3ccc(-c4ccc5oc6c(-c7ccccc7)cccc6c5c4)cc3)c3ccc4oc5ccccc5c4c3)cc2-c2ccccc2)cc1] 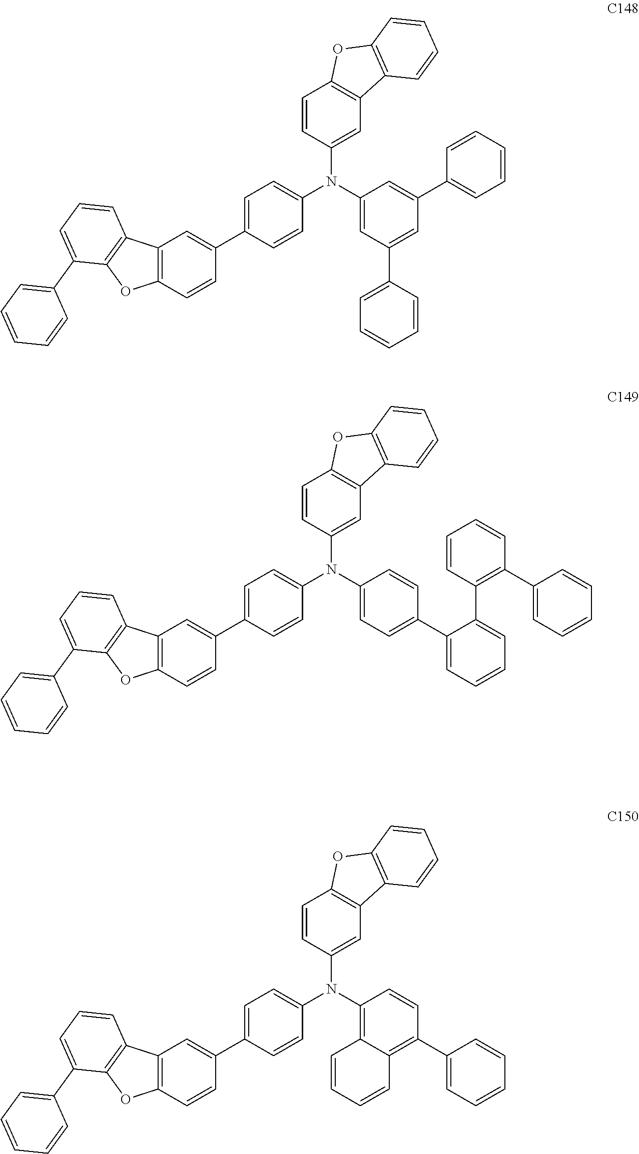 [SMILES: c1ccc(-c2cc(-c3ccccc3)cc(N(c3ccc(-c4ccc5oc6c(-c7ccccc7)cccc6c5c4)cc3)c3ccc4oc5ccccc5c4c3)c2)cc1 ; c1ccc(-c2ccccc2-c2ccccc2-c2ccc(N(c3ccc(-c4ccc5oc6c(-c7ccccc7)cccc6c5c4)cc3)c3ccc4oc5ccccc5c4c3)cc2)cc1 ; c1ccc(-c2ccc(N(c3ccc(-c4ccc5oc6c(-c7ccccc7)cccc6c5c4)cc3)c3ccc4oc5ccccc5c4c3)c3ccccc23)cc1] 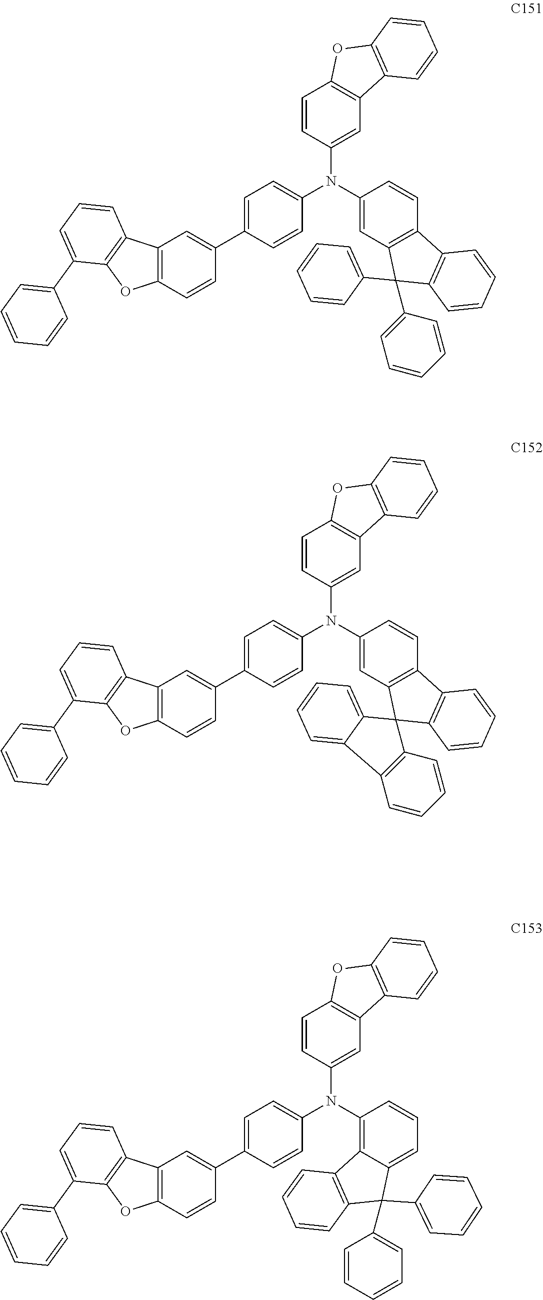 [SMILES: c1ccc(-c2cccc3c2oc2ccc(-c4ccc(N(c5ccc6c(c5)C(c5ccccc5)(c5ccccc5)c5ccccc5-6)c5ccc6oc7ccccc7c6c5)cc4)cc23)cc1 ; c1ccc(-c2cccc3c2oc2ccc(-c4ccc(N(c5ccc6c(c5)C5(c7ccccc7-c7ccccc75)c5ccccc5-6)c5ccc6oc7ccccc7c6c5)cc4)cc23)cc1 ; c1ccc(-c2cccc3c2oc2ccc(-c4ccc(N(c5ccc6oc7ccccc7c6c5)c5cccc6c5-c5ccccc5C6(c5ccccc5)c5ccccc5)cc4)cc23)cc1] 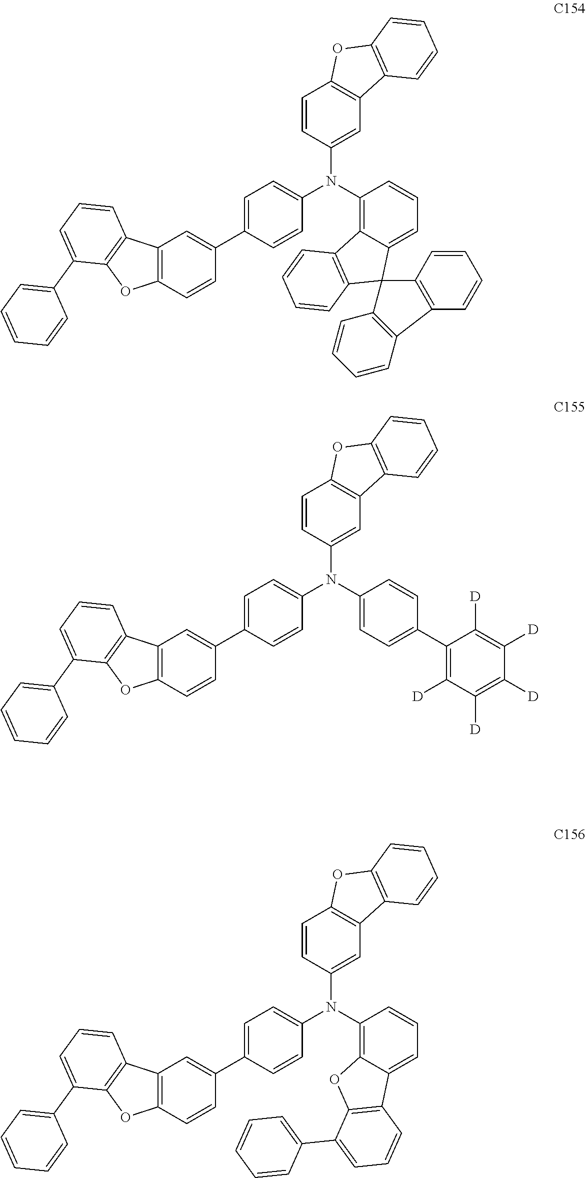 [SMILES: c1ccc(-c2cccc3c2oc2ccc(-c4ccc(N(c5ccc6oc7ccccc7c6c5)c5cccc6c5-c5ccccc5C65c6ccccc6-c6ccccc65)cc4)cc23)cc1 ; [2H]c1c([2H])c([2H])c(-c2ccc(N(c3ccc(-c4ccc5oc6c(-c7ccccc7)cccc6c5c4)cc3)c3ccc4oc5ccccc5c4c3)cc2)c([2H])c1[2H] ; c1ccc(-c2cccc3c2oc2ccc(-c4ccc(N(c5ccc6oc7ccccc7c6c5)c5cccc6c5oc5c(-c7ccccc7)cccc56)cc4)cc23)cc1] 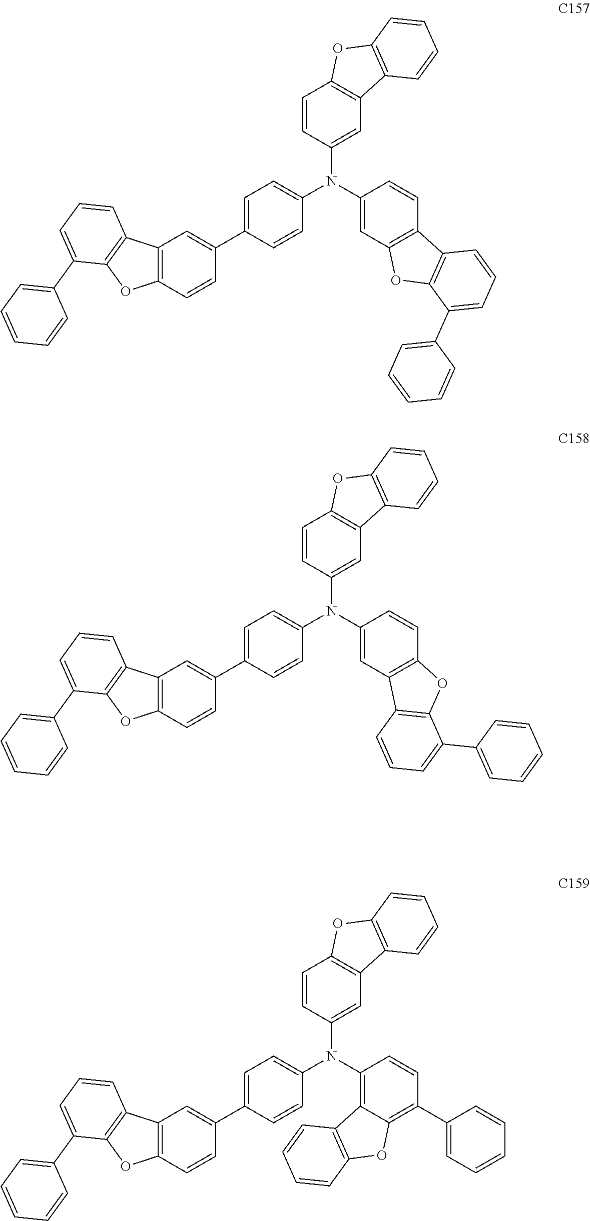 [SMILES: c1ccc(-c2cccc3c2oc2cc(N(c4ccc(-c5ccc6oc7c(-c8ccccc8)cccc7c6c5)cc4)c4ccc5oc6ccccc6c5c4)ccc23)cc1 ; c1ccc(-c2cccc3c2oc2ccc(-c4ccc(N(c5ccc6oc7ccccc7c6c5)c5ccc6oc7c(-c8ccccc8)cccc7c6c5)cc4)cc23)cc1 ; c1ccc(-c2cccc3c2oc2ccc(-c4ccc(N(c5ccc6oc7ccccc7c6c5)c5ccc(-c6ccccc6)c6oc7ccccc7c56)cc4)cc23)cc1] 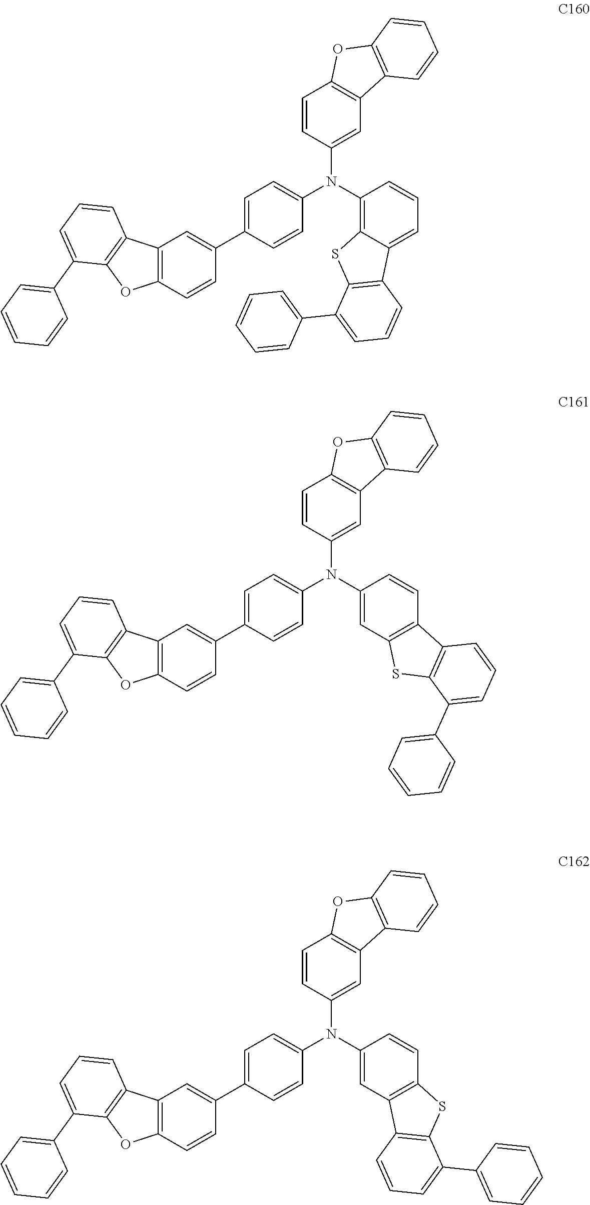 [SMILES: c1ccc(-c2cccc3c2oc2ccc(-c4ccc(N(c5ccc6oc7ccccc7c6c5)c5cccc6c5sc5c(-c7ccccc7)cccc56)cc4)cc23)cc1 ; c1ccc(-c2cccc3c2oc2ccc(-c4ccc(N(c5ccc6c(c5)sc5c(-c7ccccc7)cccc56)c5ccc6oc7ccccc7c6c5)cc4)cc23)cc1 ; c1ccc(-c2cccc3c2oc2ccc(-c4ccc(N(c5ccc6oc7ccccc7c6c5)c5ccc6sc7c(-c8ccccc8)cccc7c6c5)cc4)cc23)cc1] 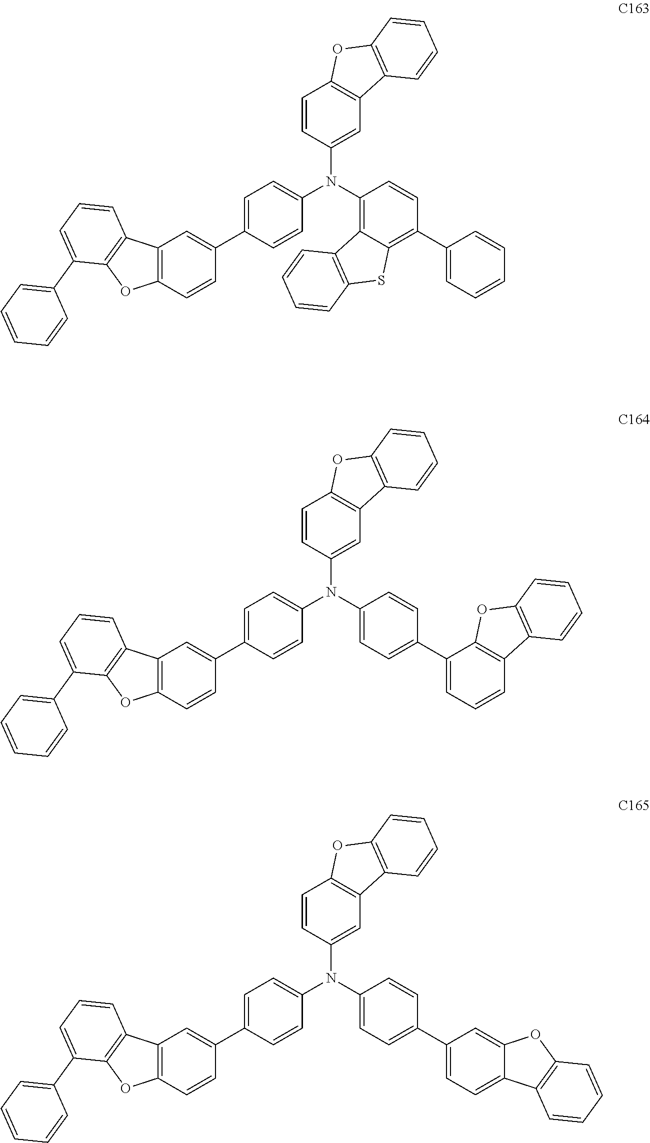 [SMILES: c1ccc(-c2cccc3c2oc2ccc(-c4ccc(N(c5ccc6oc7ccccc7c6c5)c5ccc(-c6ccccc6)c6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc6oc7ccccc7c6c5)cc4)cc23)cc1 ; c1ccc(-c2cccc3c2oc2ccc(-c4ccc(N(c5ccc(-c6ccc7c(c6)oc6ccccc67)cc5)c5ccc6oc7ccccc7c6c5)cc4)cc23)cc1] 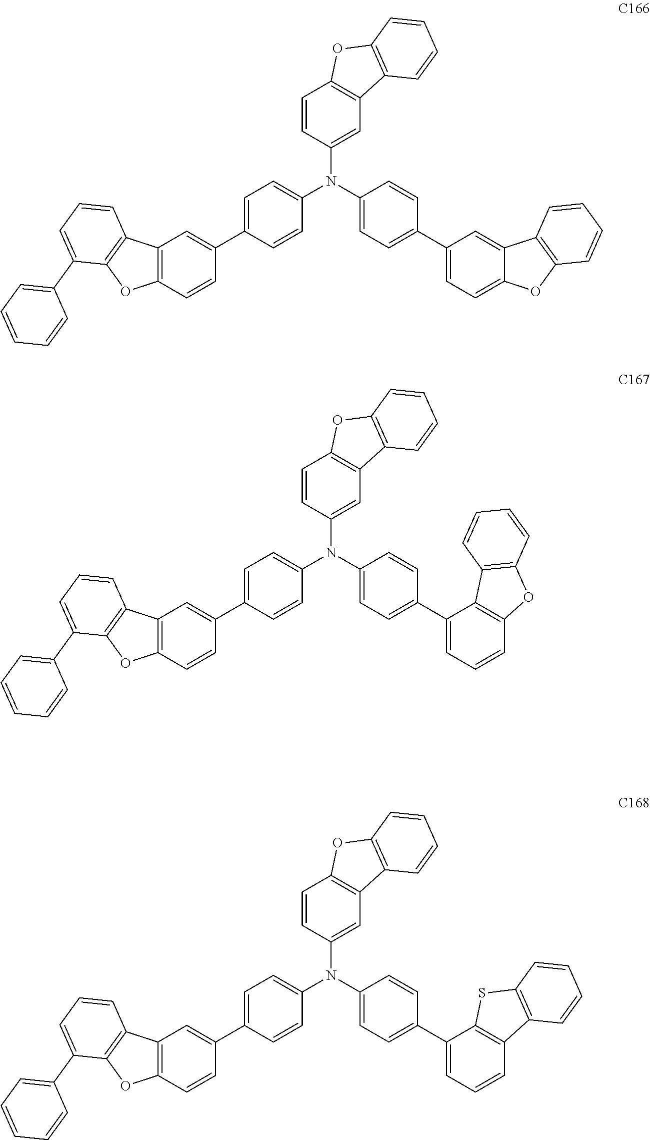 [SMILES: c1ccc(-c2cccc3c2oc2ccc(-c4ccc(N(c5ccc(-c6ccc7oc8ccccc8c7c6)cc5)c5ccc6oc7ccccc7c6c5)cc4)cc23)cc1 ; c1ccc(-c2cccc3c2oc2ccc(-c4ccc(N(c5ccc(-c6cccc7oc8ccccc8c67)cc5)c5ccc6oc7ccccc7c6c5)cc4)cc23)cc1 ; c1ccc(-c2cccc3c2oc2ccc(-c4ccc(N(c5ccc(-c6cccc7c6sc6ccccc67)cc5)c5ccc6oc7ccccc7c6c5)cc4)cc23)cc1] 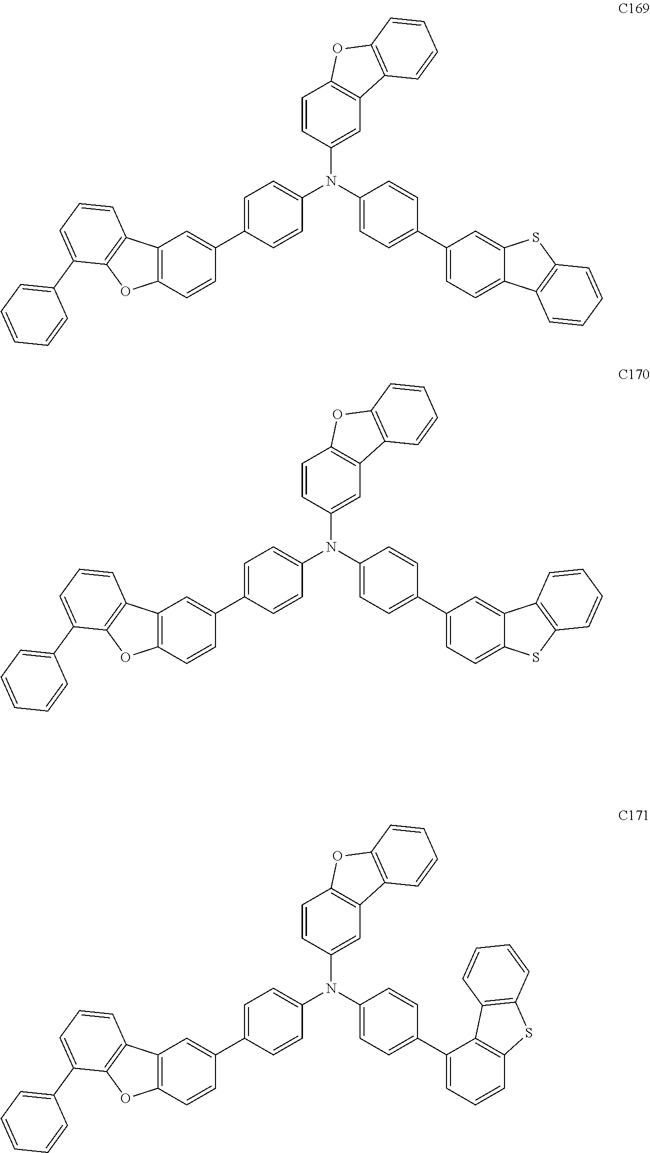 [SMILES: c1ccc(-c2cccc3c2oc2ccc(-c4ccc(N(c5ccc(-c6ccc7c(c6)sc6ccccc67)cc5)c5ccc6oc7ccccc7c6c5)cc4)cc23)cc1 ; c1ccc(-c2cccc3c2oc2ccc(-c4ccc(N(c5ccc(-c6ccc7sc8ccccc8c7c6)cc5)c5ccc6oc7ccccc7c6c5)cc4)cc23)cc1 ; c1ccc(-c2cccc3c2oc2ccc(-c4ccc(N(c5ccc(-c6cccc7sc8ccccc8c67)cc5)c5ccc6oc7ccccc7c6c5)cc4)cc23)cc1] 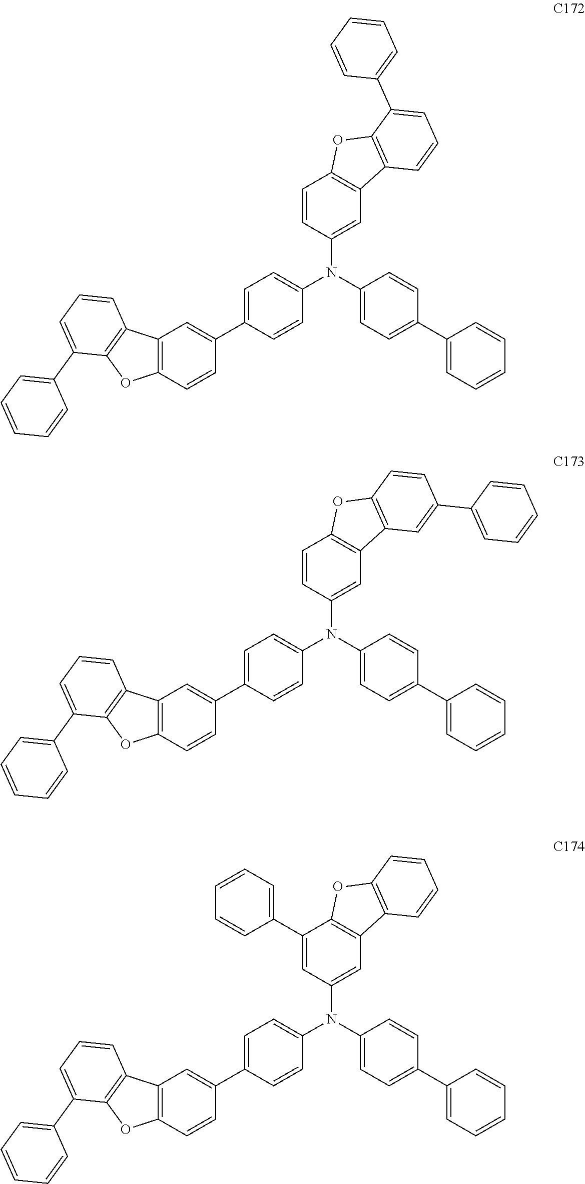 [SMILES: c1ccc(-c2ccc(N(c3ccc(-c4ccc5oc6c(-c7ccccc7)cccc6c5c4)cc3)c3ccc4oc5c(-c6ccccc6)cccc5c4c3)cc2)cc1 ; c1ccc(-c2ccc(N(c3ccc(-c4ccc5oc6c(-c7ccccc7)cccc6c5c4)cc3)c3ccc4oc5ccc(-c6ccccc6)cc5c4c3)cc2)cc1 ; c1ccc(-c2ccc(N(c3ccc(-c4ccc5oc6c(-c7ccccc7)cccc6c5c4)cc3)c3cc(-c4ccccc4)c4oc5ccccc5c4c3)cc2)cc1] 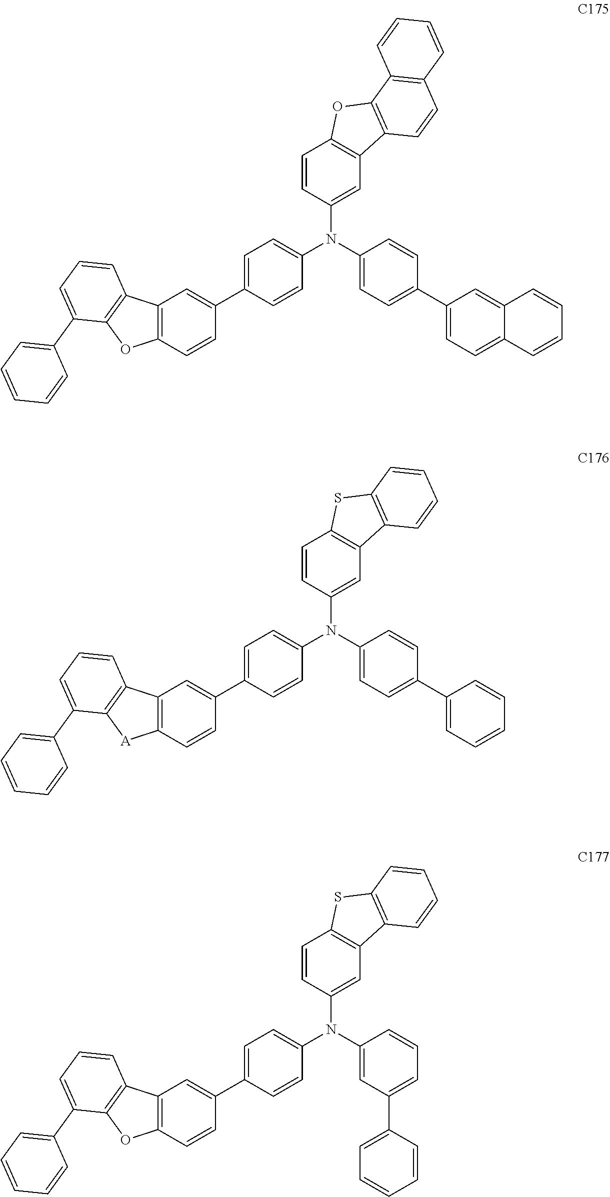 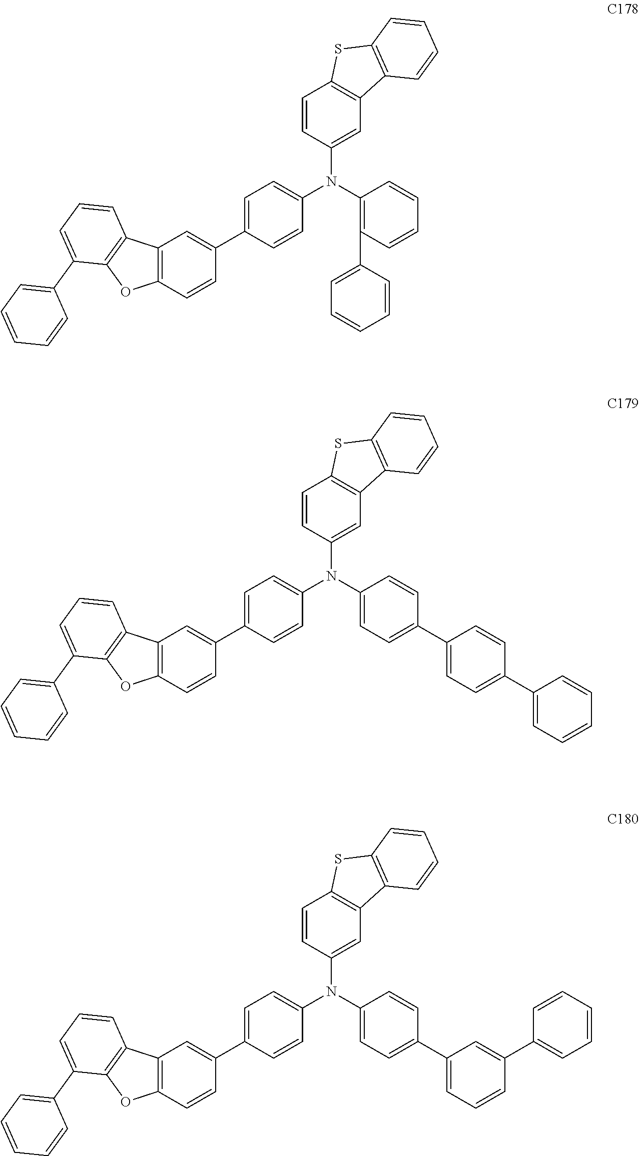 [SMILES: c1ccc(-c2ccccc2N(c2ccc(-c3ccc4oc5c(-c6ccccc6)cccc5c4c3)cc2)c2ccc3sc4ccccc4c3c2)cc1 ; c1ccc(-c2ccc(-c3ccc(N(c4ccc(-c5ccc6oc7c(-c8ccccc8)cccc7c6c5)cc4)c4ccc5sc6ccccc6c5c4)cc3)cc2)cc1 ; c1ccc(-c2cccc(-c3ccc(N(c4ccc(-c5ccc6oc7c(-c8ccccc8)cccc7c6c5)cc4)c4ccc5sc6ccccc6c5c4)cc3)c2)cc1] 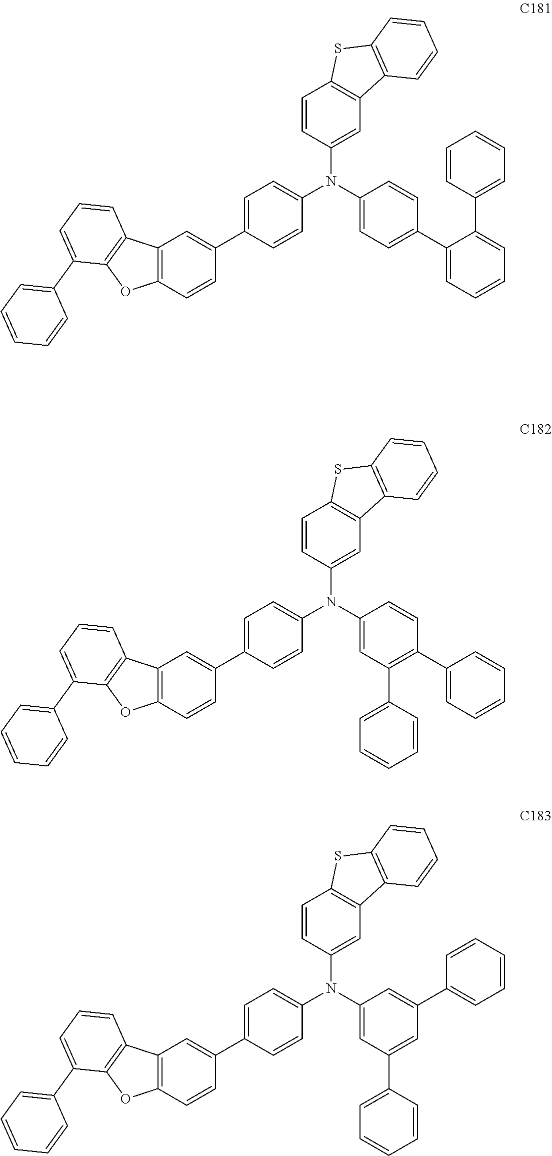 [SMILES: c1ccc(-c2ccccc2-c2ccc(N(c3ccc(-c4ccc5oc6c(-c7ccccc7)cccc6c5c4)cc3)c3ccc4sc5ccccc5c4c3)cc2)cc1 ; c1ccc(-c2ccc(N(c3ccc(-c4ccc5oc6c(-c7ccccc7)cccc6c5c4)cc3)c3ccc4sc5ccccc5c4c3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc4sc5ccccc5c4c3)c2)cc1] 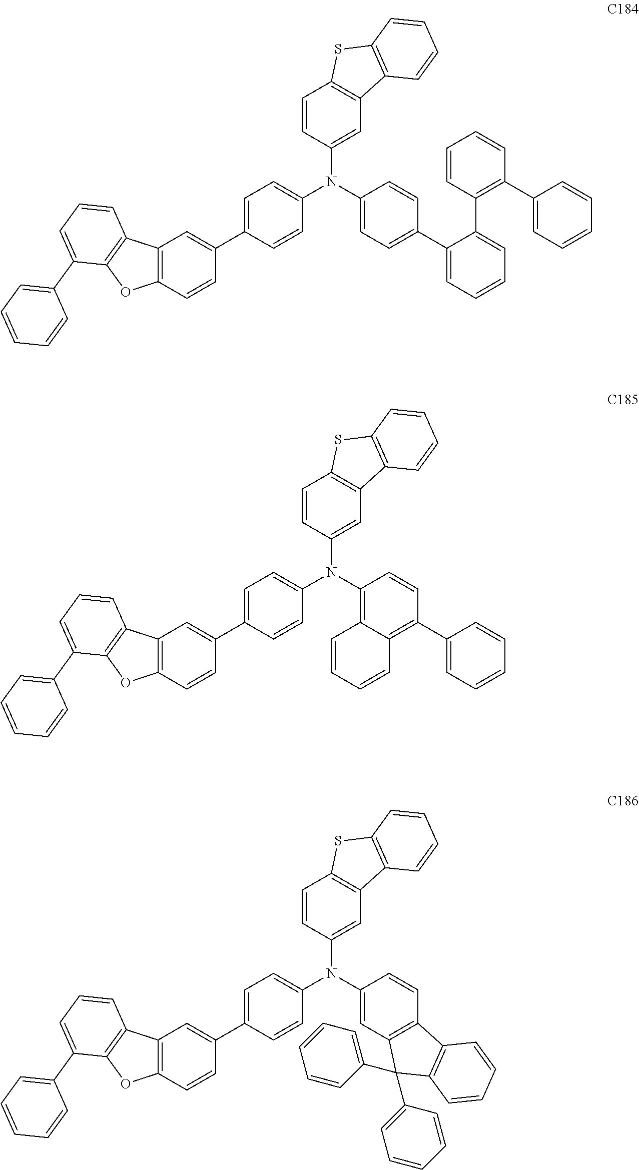 [SMILES: c1ccc(-c2ccccc2-c2ccccc2-c2ccc(N(c3ccc(-c4ccc5oc6c(-c7ccccc7)cccc6c5c4)cc3)c3ccc4sc5ccccc5c4c3)cc2)cc1 ; c1ccc(-c2ccc(N(c3ccc(-c4ccc5oc6c(-c7ccccc7)cccc6c5c4)cc3)c3ccc4sc5ccccc5c4c3)c3ccccc23)cc1 ; c1ccc(-c2cccc3c2oc2ccc(-c4ccc(N(c5ccc6c(c5)C(c5ccccc5)(c5ccccc5)c5ccccc5-6)c5ccc6sc7ccccc7c6c5)cc4)cc23)cc1] 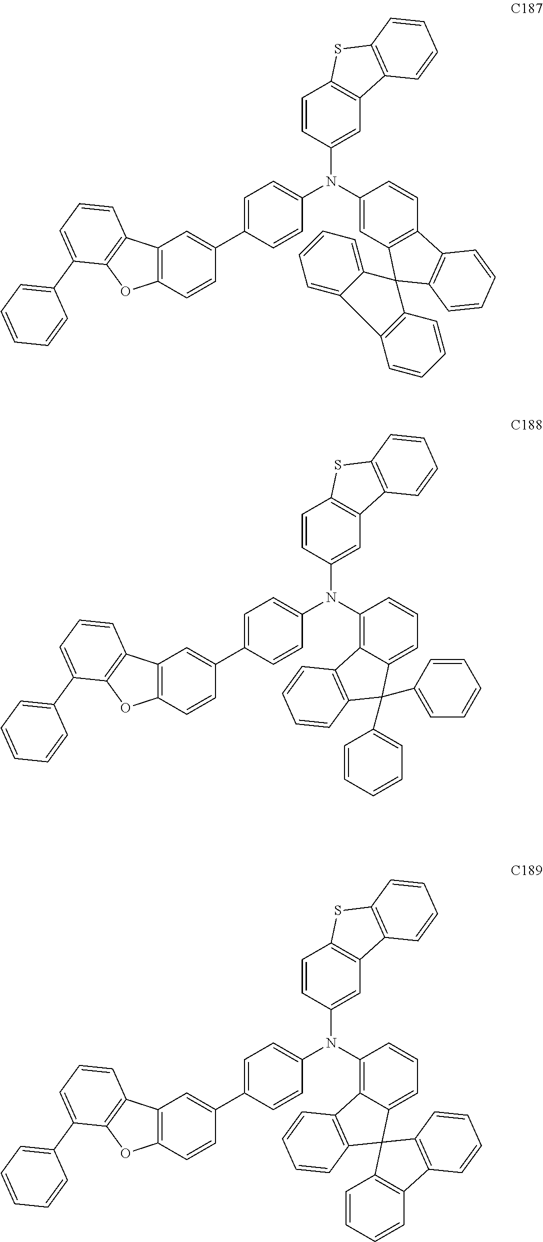 [SMILES: c1ccc(-c2cccc3c2oc2ccc(-c4ccc(N(c5ccc6c(c5)C5(c7ccccc7-c7ccccc75)c5ccccc5-6)c5ccc6sc7ccccc7c6c5)cc4)cc23)cc1 ; c1ccc(-c2cccc3c2oc2ccc(-c4ccc(N(c5ccc6sc7ccccc7c6c5)c5cccc6c5-c5ccccc5C6(c5ccccc5)c5ccccc5)cc4)cc23)cc1 ; c1ccc(-c2cccc3c2oc2ccc(-c4ccc(N(c5ccc6sc7ccccc7c6c5)c5cccc6c5-c5ccccc5C65c6ccccc6-c6ccccc65)cc4)cc23)cc1] 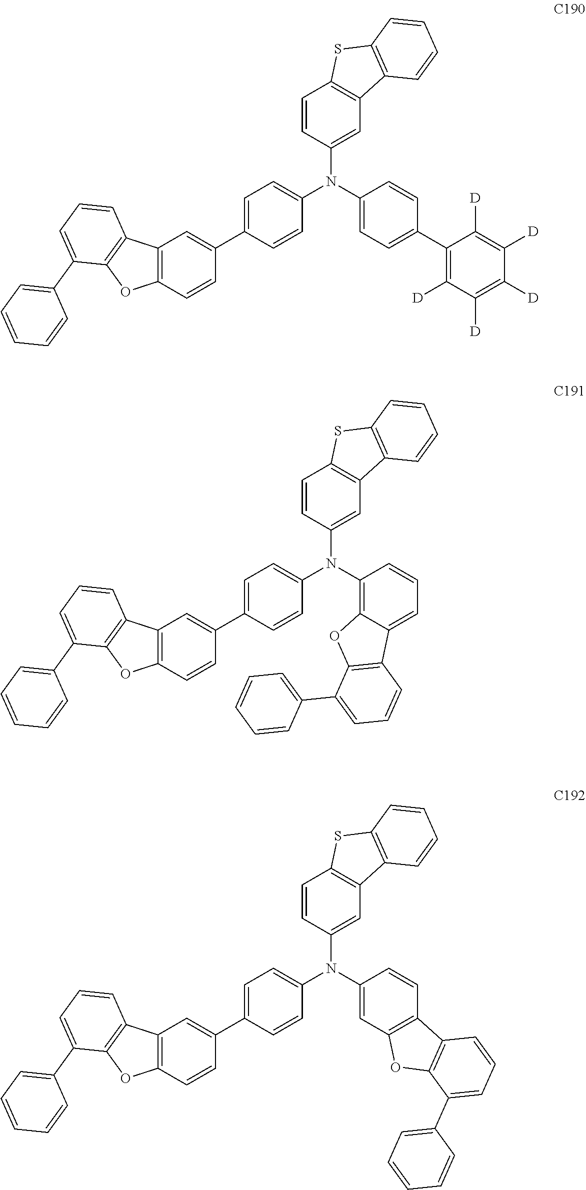 [SMILES: [2H]c1c([2H])c([2H])c(-c2ccc(N(c3ccc(-c4ccc5oc6c(-c7ccccc7)cccc6c5c4)cc3)c3ccc4sc5ccccc5c4c3)cc2)c([2H])c1[2H] ; c1ccc(-c2cccc3c2oc2ccc(-c4ccc(N(c5ccc6sc7ccccc7c6c5)c5cccc6c5oc5c(-c7ccccc7)cccc56)cc4)cc23)cc1 ; c1ccc(-c2cccc3c2oc2cc(N(c4ccc(-c5ccc6oc7c(-c8ccccc8)cccc7c6c5)cc4)c4ccc5sc6ccccc6c5c4)ccc23)cc1] 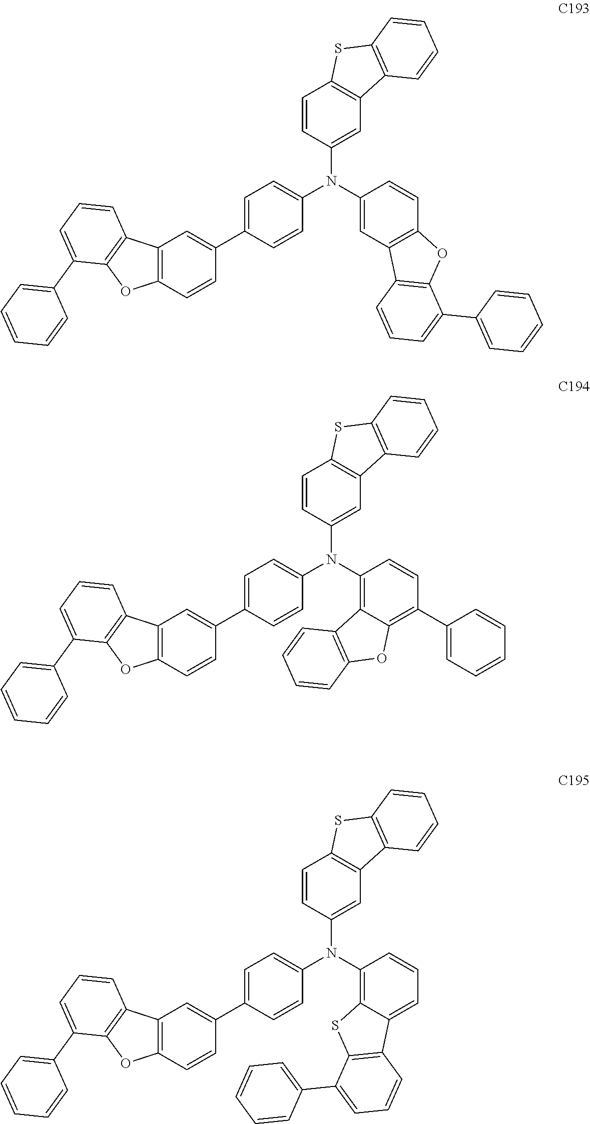 [SMILES: c1ccc(-c2cccc3c2oc2ccc(-c4ccc(N(c5ccc6oc7c(-c8ccccc8)cccc7c6c5)c5ccc6sc7ccccc7c6c5)cc4)cc23)cc1 ; c1ccc(-c2cccc3c2oc2ccc(-c4ccc(N(c5ccc6sc7ccccc7c6c5)c5ccc(-c6ccccc6)c6oc7ccccc7c56)cc4)cc23)cc1 ; c1ccc(-c2cccc3c2oc2ccc(-c4ccc(N(c5ccc6sc7ccccc7c6c5)c5cccc6c5sc5c(-c7ccccc7)cccc56)cc4)cc23)cc1] 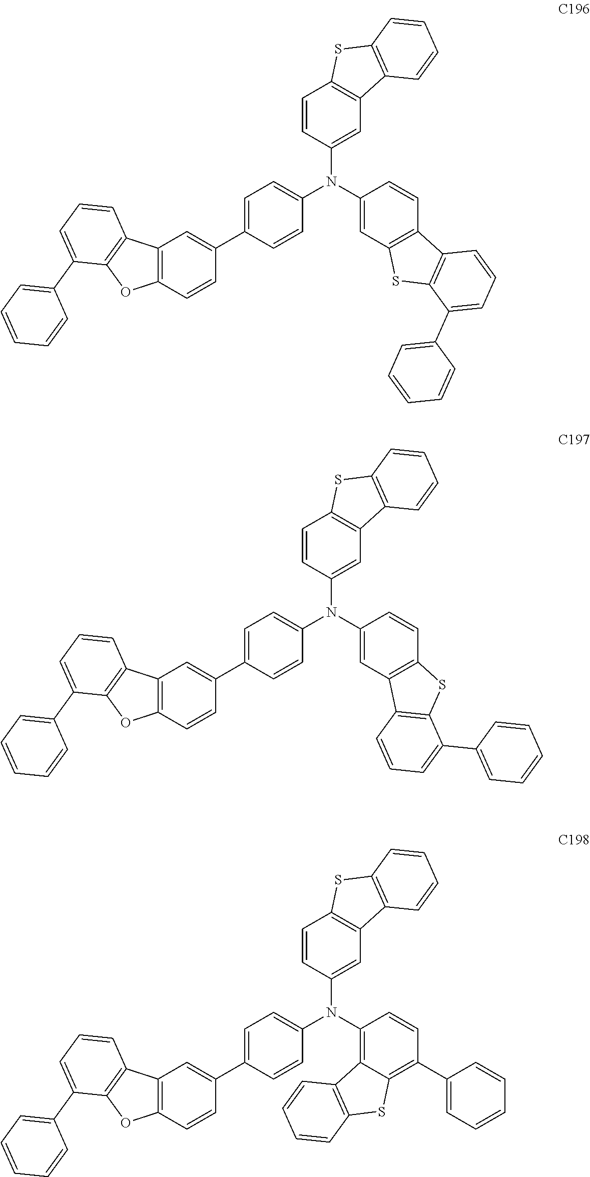 [SMILES: c1ccc(-c2cccc3c2oc2ccc(-c4ccc(N(c5ccc6c(c5)sc5c(-c7ccccc7)cccc56)c5ccc6sc7ccccc7c6c5)cc4)cc23)cc1 ; c1ccc(-c2cccc3c2oc2ccc(-c4ccc(N(c5ccc6sc7ccccc7c6c5)c5ccc6sc7c(-c8ccccc8)cccc7c6c5)cc4)cc23)cc1 ; c1ccc(-c2cccc3c2oc2ccc(-c4ccc(N(c5ccc6sc7ccccc7c6c5)c5ccc(-c6ccccc6)c6sc7ccccc7c56)cc4)cc23)cc1] 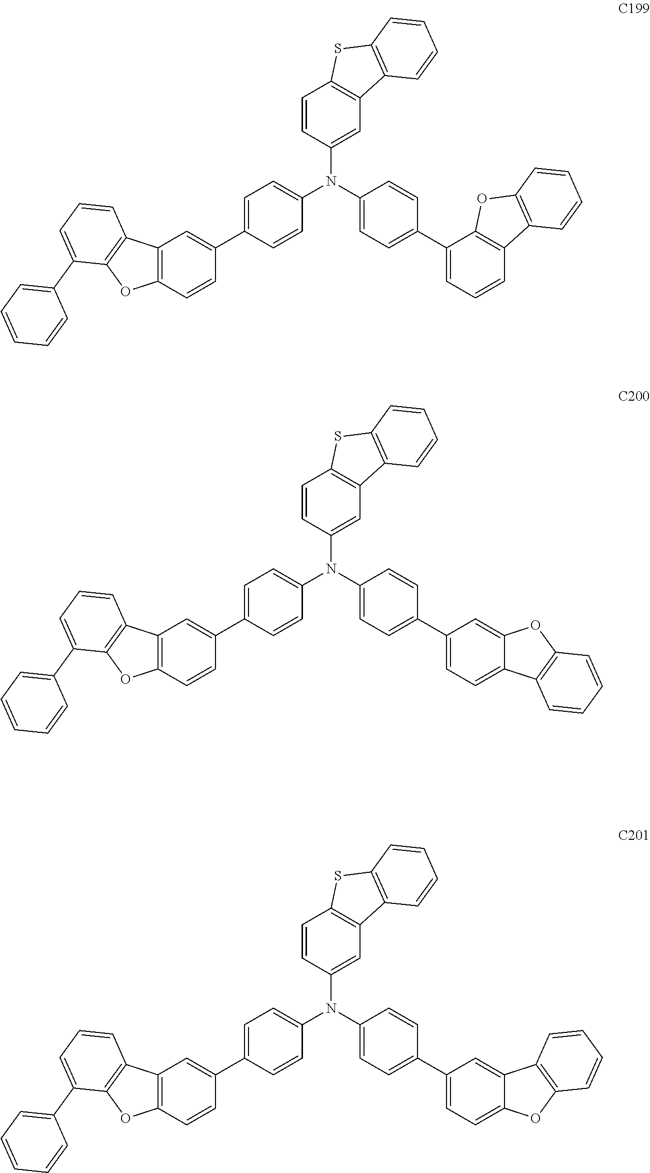 [SMILES: c1ccc(-c2cccc3c2oc2ccc(-c4ccc(N(c5ccc(-c6cccc7c6oc6ccccc67)cc5)c5ccc6sc7ccccc7c6c5)cc4)cc23)cc1 ; c1ccc(-c2cccc3c2oc2ccc(-c4ccc(N(c5ccc(-c6ccc7c(c6)oc6ccccc67)cc5)c5ccc6sc7ccccc7c6c5)cc4)cc23)cc1 ; c1ccc(-c2cccc3c2oc2ccc(-c4ccc(N(c5ccc(-c6ccc7oc8ccccc8c7c6)cc5)c5ccc6sc7ccccc7c6c5)cc4)cc23)cc1] 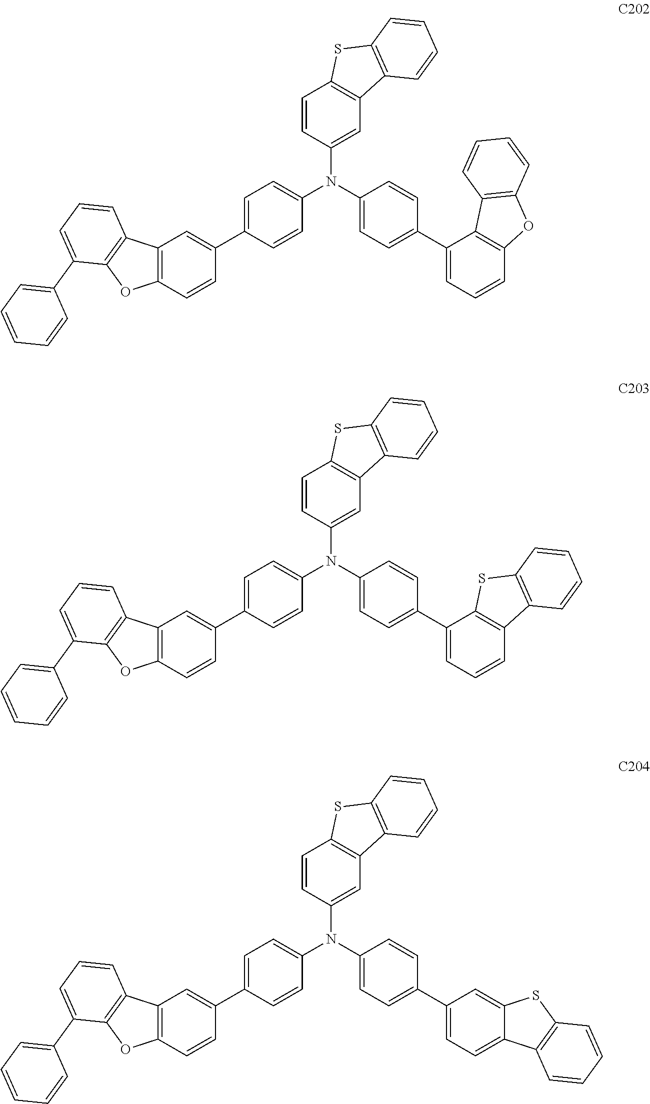 [SMILES: c1ccc(-c2cccc3c2oc2ccc(-c4ccc(N(c5ccc(-c6cccc7oc8ccccc8c67)cc5)c5ccc6sc7ccccc7c6c5)cc4)cc23)cc1 ; c1ccc(-c2cccc3c2oc2ccc(-c4ccc(N(c5ccc(-c6cccc7c6sc6ccccc67)cc5)c5ccc6sc7ccccc7c6c5)cc4)cc23)cc1 ; c1ccc(-c2cccc3c2oc2ccc(-c4ccc(N(c5ccc(-c6ccc7c(c6)sc6ccccc67)cc5)c5ccc6sc7ccccc7c6c5)cc4)cc23)cc1] 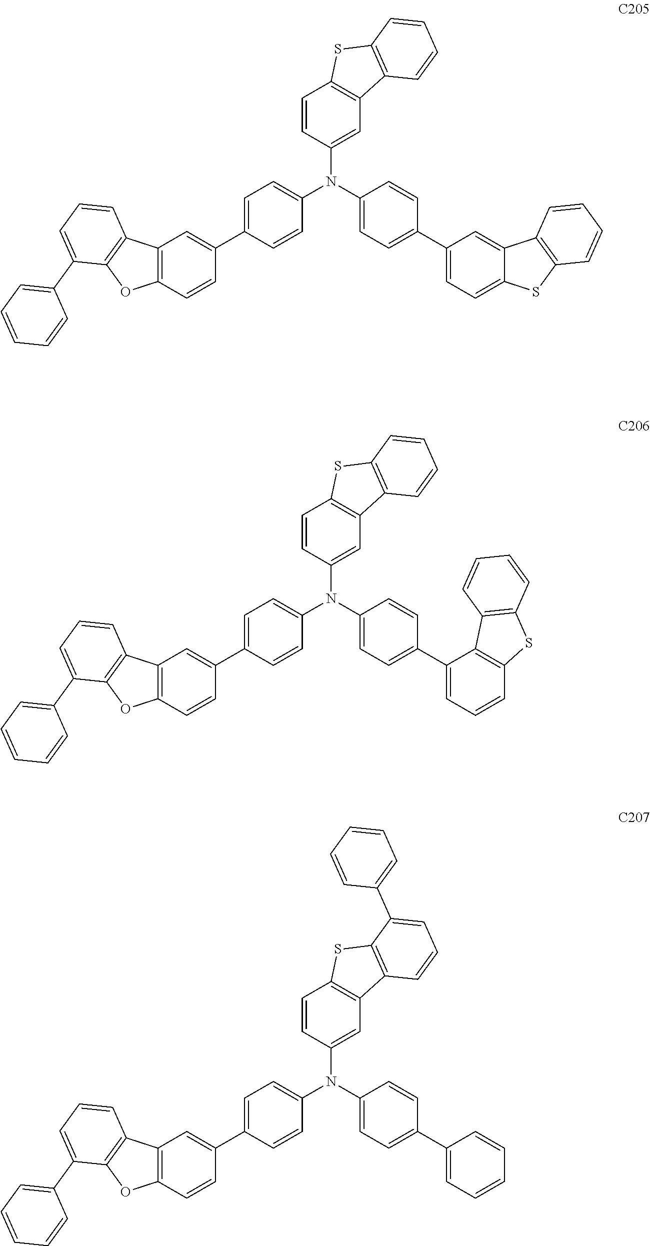 [SMILES: c1ccc(-c2cccc3c2oc2ccc(-c4ccc(N(c5ccc(-c6ccc7sc8ccccc8c7c6)cc5)c5ccc6sc7ccccc7c6c5)cc4)cc23)cc1 ; c1ccc(-c2cccc3c2oc2ccc(-c4ccc(N(c5ccc(-c6cccc7sc8ccccc8c67)cc5)c5ccc6sc7ccccc7c6c5)cc4)cc23)cc1 ; c1ccc(-c2ccc(N(c3ccc(-c4ccc5oc6c(-c7ccccc7)cccc6c5c4)cc3)c3ccc4sc5c(-c6ccccc6)cccc5c4c3)cc2)cc1] 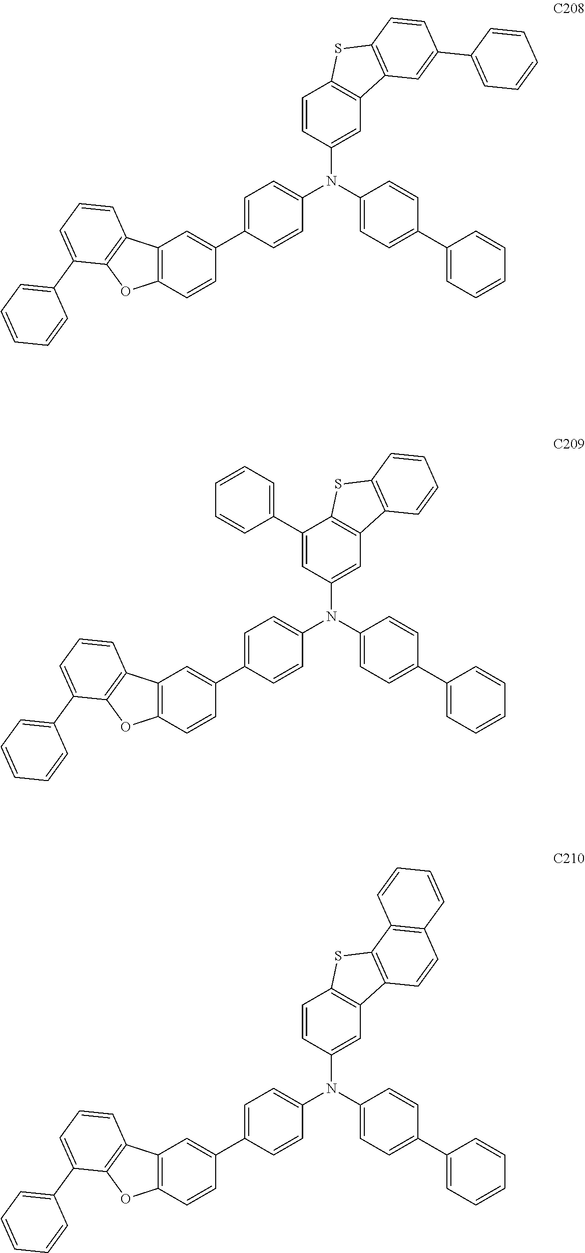 [SMILES: c1ccc(-c2ccc(N(c3ccc(-c4ccc5oc6c(-c7ccccc7)cccc6c5c4)cc3)c3ccc4sc5ccc(-c6ccccc6)cc5c4c3)cc2)cc1 ; c1ccc(-c2ccc(N(c3ccc(-c4ccc5oc6c(-c7ccccc7)cccc6c5c4)cc3)c3cc(-c4ccccc4)c4sc5ccccc5c4c3)cc2)cc1 ; c1ccc(-c2ccc(N(c3ccc(-c4ccc5oc6c(-c7ccccc7)cccc6c5c4)cc3)c3ccc4sc5c6ccccc6ccc5c4c3)cc2)cc1] 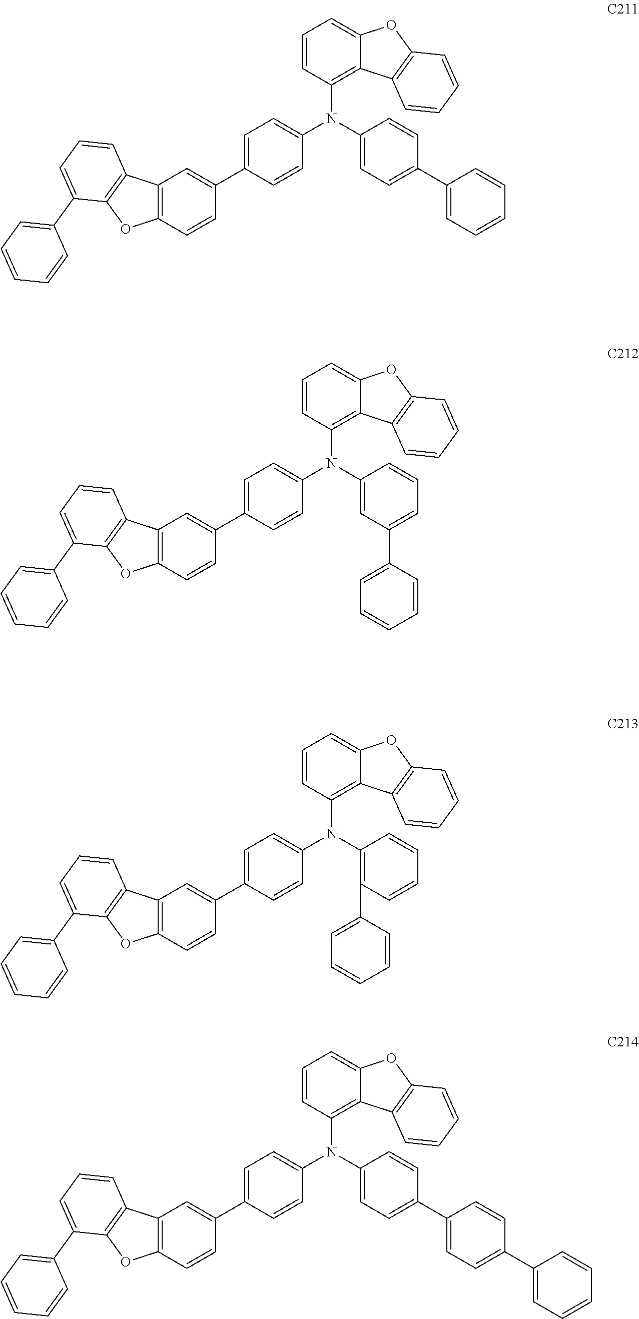 [SMILES: c1ccc(-c2ccc(N(c3ccc(-c4ccc5oc6c(-c7ccccc7)cccc6c5c4)cc3)c3cccc4oc5ccccc5c34)cc2)cc1 ; c1ccc(-c2cccc(N(c3ccc(-c4ccc5oc6c(-c7ccccc7)cccc6c5c4)cc3)c3cccc4oc5ccccc5c34)c2)cc1 ; c1ccc(-c2ccccc2N(c2ccc(-c3ccc4oc5c(-c6ccccc6)cccc5c4c3)cc2)c2cccc3oc4ccccc4c23)cc1 ; c1ccc(-c2ccc(-c3ccc(N(c4ccc(-c5ccc6oc7c(-c8ccccc8)cccc7c6c5)cc4)c4cccc5oc6ccccc6c45)cc3)cc2)cc1] 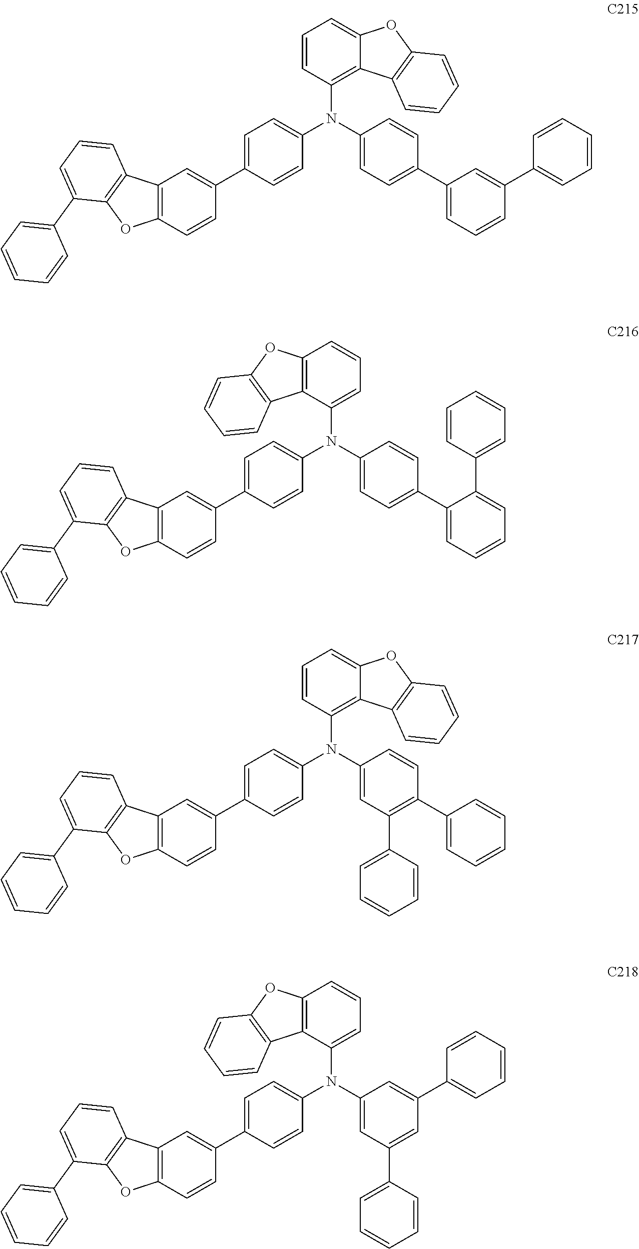 [SMILES: c1ccc(-c2cccc(-c3ccc(N(c4ccc(-c5ccc6oc7c(-c8ccccc8)cccc7c6c5)cc4)c4cccc5oc6ccccc6c45)cc3)c2)cc1 ; c1ccc(-c2ccccc2-c2ccc(N(c3ccc(-c4ccc5oc6c(-c7ccccc7)cccc6c5c4)cc3)c3cccc4oc5ccccc5c34)cc2)cc1 ; c1ccc(-c2ccc(N(c3ccc(-c4ccc5oc6c(-c7ccccc7)cccc6c5c4)cc3)c3cccc4oc5ccccc5c34)cc2-c2ccccc2)cc1 ; c1ccc(-c2cc(-c3ccccc3)cc(N(c3ccc(-c4ccc5oc6c(-c7ccccc7)cccc6c5c4)cc3)c3cccc4oc5ccccc5c34)c2)cc1] 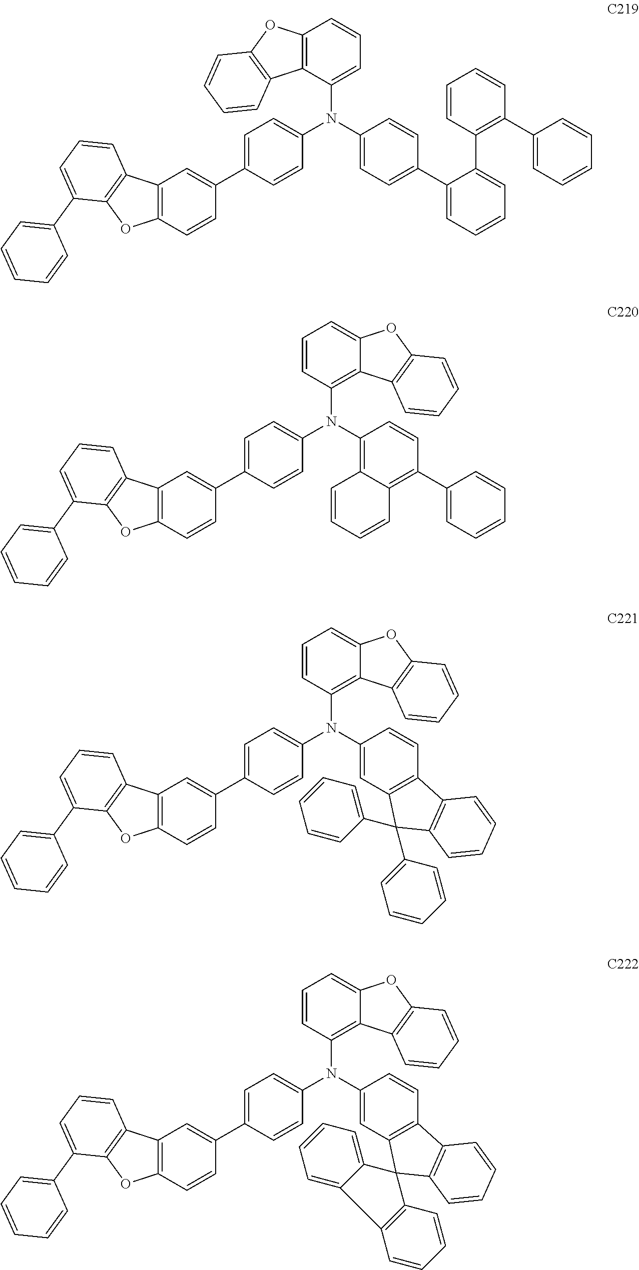 [SMILES: c1ccc(-c2ccccc2-c2ccccc2-c2ccc(N(c3ccc(-c4ccc5oc6c(-c7ccccc7)cccc6c5c4)cc3)c3cccc4oc5ccccc5c34)cc2)cc1 ; c1ccc(-c2ccc(N(c3ccc(-c4ccc5oc6c(-c7ccccc7)cccc6c5c4)cc3)c3cccc4oc5ccccc5c34)c3ccccc23)cc1 ; c1ccc(-c2cccc3c2oc2ccc(-c4ccc(N(c5ccc6c(c5)C(c5ccccc5)(c5ccccc5)c5ccccc5-6)c5cccc6oc7ccccc7c56)cc4)cc23)cc1 ; c1ccc(-c2cccc3c2oc2ccc(-c4ccc(N(c5ccc6c(c5)C5(c7ccccc7-c7ccccc75)c5ccccc5-6)c5cccc6oc7ccccc7c56)cc4)cc23)cc1] 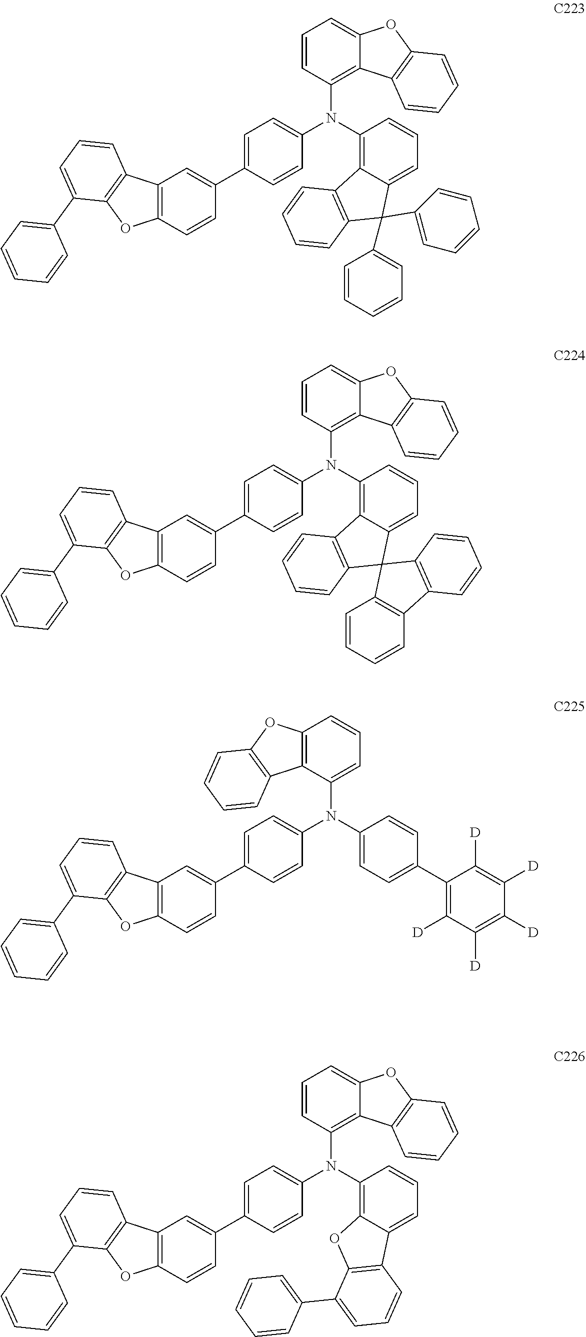 [SMILES: c1ccc(-c2cccc3c2oc2ccc(-c4ccc(N(c5cccc6c5-c5ccccc5C6(c5ccccc5)c5ccccc5)c5cccc6oc7ccccc7c56)cc4)cc23)cc1 ; c1ccc(-c2cccc3c2oc2ccc(-c4ccc(N(c5cccc6c5-c5ccccc5C65c6ccccc6-c6ccccc65)c5cccc6oc7ccccc7c56)cc4)cc23)cc1 ; [2H]c1c([2H])c([2H])c(-c2ccc(N(c3ccc(-c4ccc5oc6c(-c7ccccc7)cccc6c5c4)cc3)c3cccc4oc5ccccc5c34)cc2)c([2H])c1[2H] ; c1ccc(-c2cccc3c2oc2ccc(-c4ccc(N(c5cccc6c5oc5c(-c7ccccc7)cccc56)c5cccc6oc7ccccc7c56)cc4)cc23)cc1] 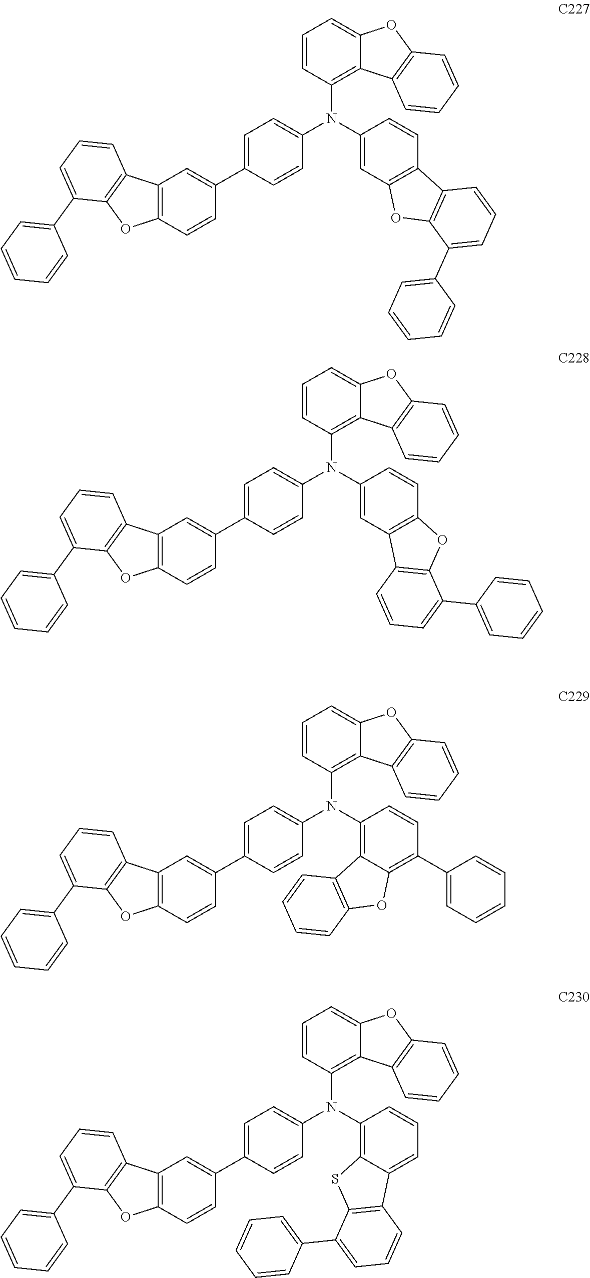 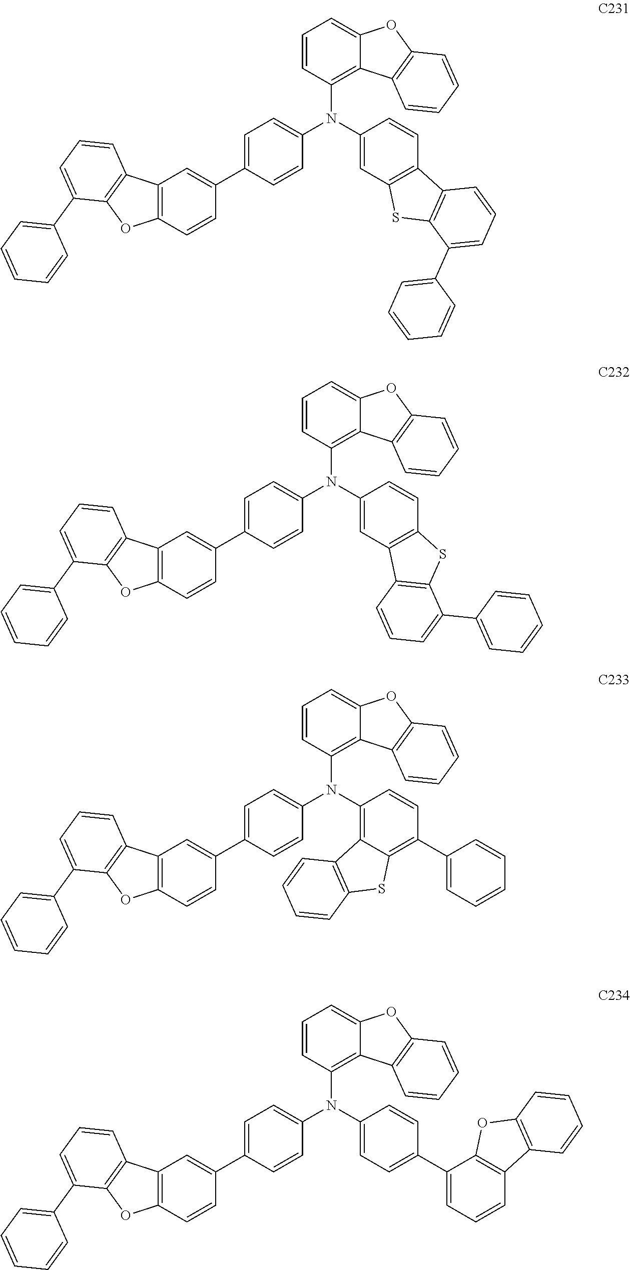 [SMILES: c1ccc(-c2cccc3c2oc2ccc(-c4ccc(N(c5ccc6c(c5)sc5c(-c7ccccc7)cccc56)c5cccc6oc7ccccc7c56)cc4)cc23)cc1 ; c1ccc(-c2cccc3c2oc2ccc(-c4ccc(N(c5ccc6sc7c(-c8ccccc8)cccc7c6c5)c5cccc6oc7ccccc7c56)cc4)cc23)cc1 ; c1ccc(-c2cccc3c2oc2ccc(-c4ccc(N(c5cccc6oc7ccccc7c56)c5ccc(-c6ccccc6)c6sc7ccccc7c56)cc4)cc23)cc1 ; c1ccc(-c2cccc3c2oc2ccc(-c4ccc(N(c5ccc(-c6cccc7c6oc6ccccc67)cc5)c5cccc6oc7ccccc7c56)cc4)cc23)cc1] 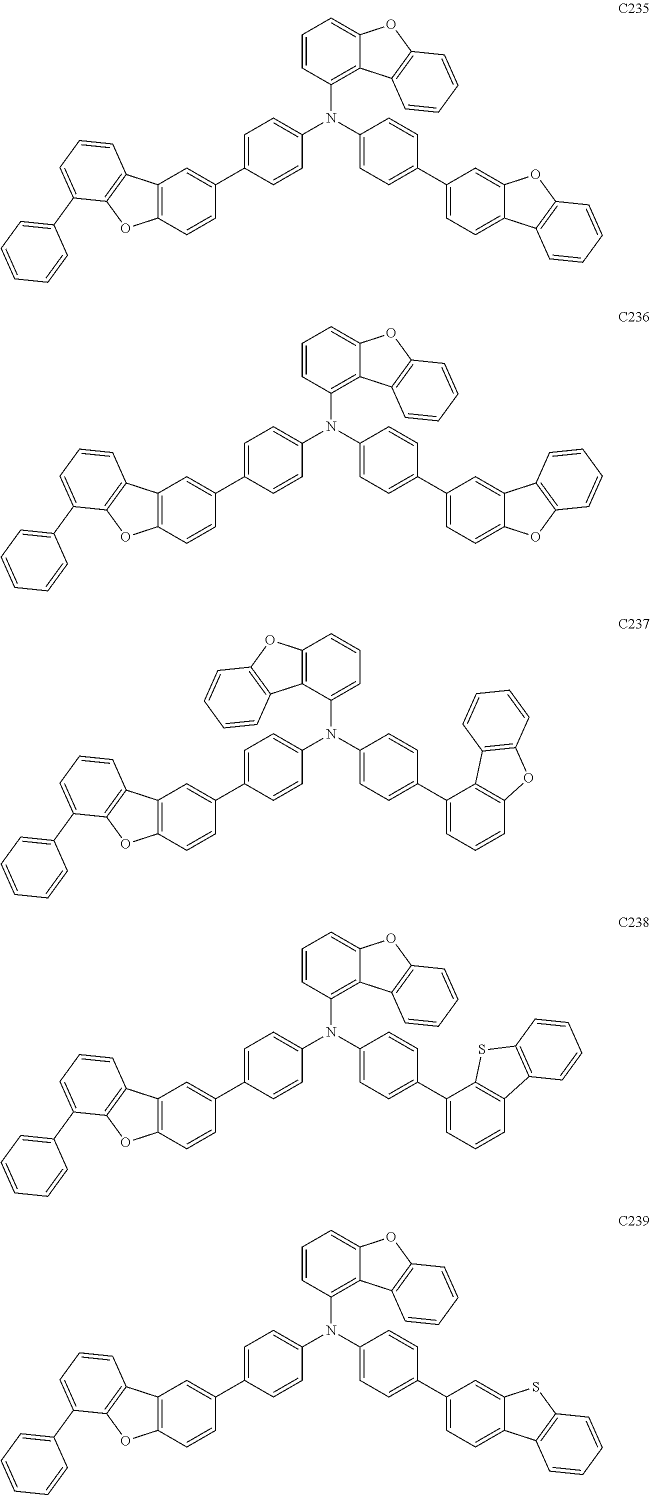 [SMILES: c1ccc(-c2cccc3c2oc2ccc(-c4ccc(N(c5ccc(-c6ccc7c(c6)oc6ccccc67)cc5)c5cccc6oc7ccccc7c56)cc4)cc23)cc1 ; c1ccc(-c2cccc3c2oc2ccc(-c4ccc(N(c5ccc(-c6ccc7oc8ccccc8c7c6)cc5)c5cccc6oc7ccccc7c56)cc4)cc23)cc1 ; c1ccc(-c2cccc3c2oc2ccc(-c4ccc(N(c5ccc(-c6cccc7oc8ccccc8c67)cc5)c5cccc6oc7ccccc7c56)cc4)cc23)cc1 ; c1ccc(-c2cccc3c2oc2ccc(-c4ccc(N(c5ccc(-c6cccc7c6sc6ccccc67)cc5)c5cccc6oc7ccccc7c56)cc4)cc23)cc1 ; c1ccc(-c2cccc3c2oc2ccc(-c4ccc(N(c5ccc(-c6ccc7c(c6)sc6ccccc67)cc5)c5cccc6oc7ccccc7c56)cc4)cc23)cc1] 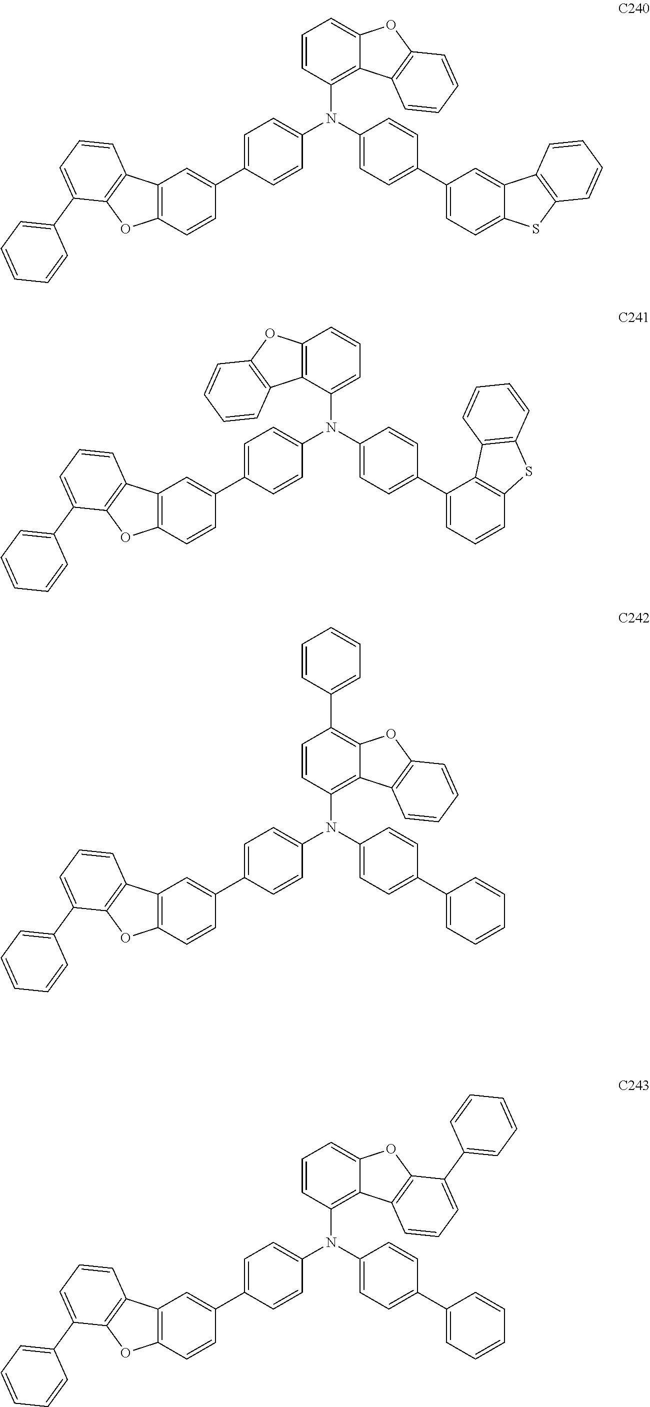 [SMILES: c1ccc(-c2cccc3c2oc2ccc(-c4ccc(N(c5ccc(-c6ccc7sc8ccccc8c7c6)cc5)c5cccc6oc7ccccc7c56)cc4)cc23)cc1 ; c1ccc(-c2cccc3c2oc2ccc(-c4ccc(N(c5ccc(-c6cccc7sc8ccccc8c67)cc5)c5cccc6oc7ccccc7c56)cc4)cc23)cc1 ; c1ccc(-c2ccc(N(c3ccc(-c4ccc5oc6c(-c7ccccc7)cccc6c5c4)cc3)c3ccc(-c4ccccc4)c4oc5ccccc5c34)cc2)cc1 ; c1ccc(-c2ccc(N(c3ccc(-c4ccc5oc6c(-c7ccccc7)cccc6c5c4)cc3)c3cccc4oc5c(-c6ccccc6)cccc5c34)cc2)cc1] 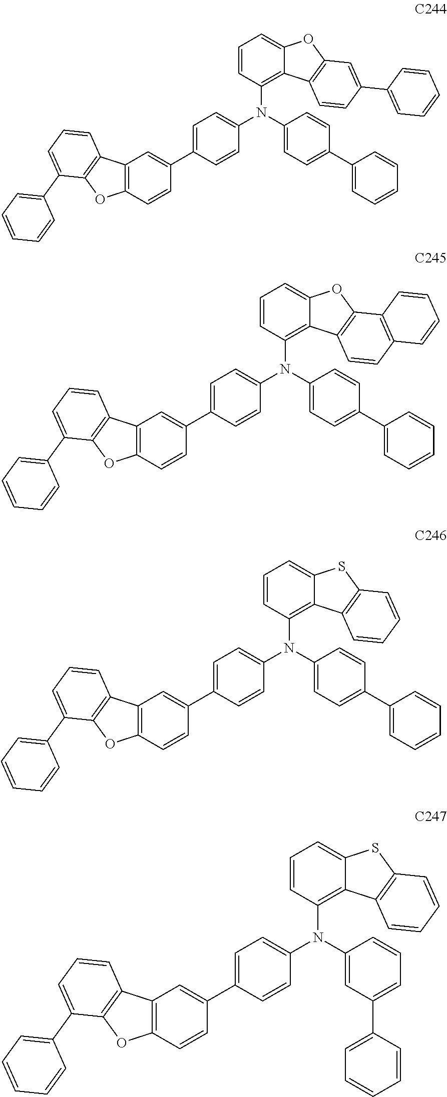 [SMILES: c1ccc(-c2ccc(N(c3ccc(-c4ccc5oc6c(-c7ccccc7)cccc6c5c4)cc3)c3cccc4oc5cc(-c6ccccc6)ccc5c34)cc2)cc1 ; c1ccc(-c2ccc(N(c3ccc(-c4ccc5oc6c(-c7ccccc7)cccc6c5c4)cc3)c3cccc4oc5c6ccccc6ccc5c34)cc2)cc1 ; c1ccc(-c2ccc(N(c3ccc(-c4ccc5oc6c(-c7ccccc7)cccc6c5c4)cc3)c3cccc4sc5ccccc5c34)cc2)cc1 ; c1ccc(-c2cccc(N(c3ccc(-c4ccc5oc6c(-c7ccccc7)cccc6c5c4)cc3)c3cccc4sc5ccccc5c34)c2)cc1] 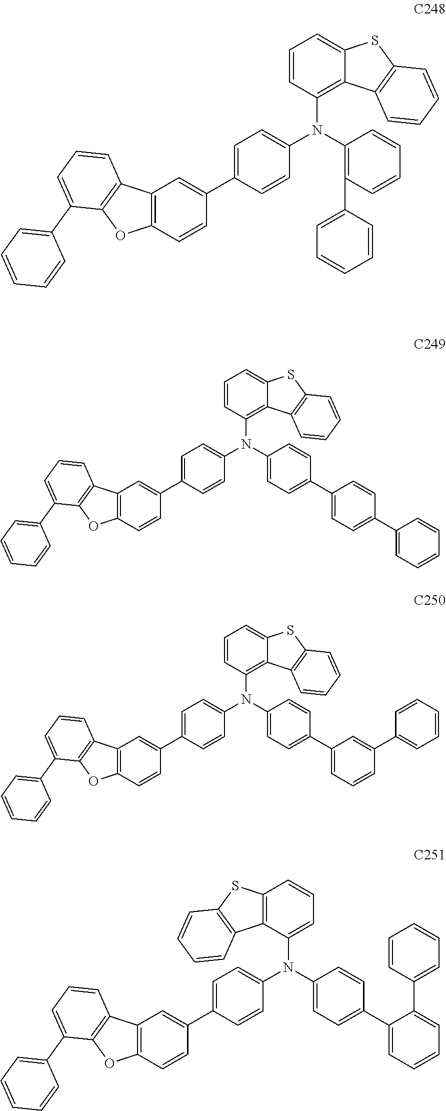 [SMILES: c1ccc(-c2ccccc2N(c2ccc(-c3ccc4oc5c(-c6ccccc6)cccc5c4c3)cc2)c2cccc3sc4ccccc4c23)cc1 ; c1ccc(-c2ccc(-c3ccc(N(c4ccc(-c5ccc6oc7c(-c8ccccc8)cccc7c6c5)cc4)c4cccc5sc6ccccc6c45)cc3)cc2)cc1 ; c1ccc(-c2cccc(-c3ccc(N(c4ccc(-c5ccc6oc7c(-c8ccccc8)cccc7c6c5)cc4)c4cccc5sc6ccccc6c45)cc3)c2)cc1 ; c1ccc(-c2ccccc2-c2ccc(N(c3ccc(-c4ccc5oc6c(-c7ccccc7)cccc6c5c4)cc3)c3cccc4sc5ccccc5c34)cc2)cc1] 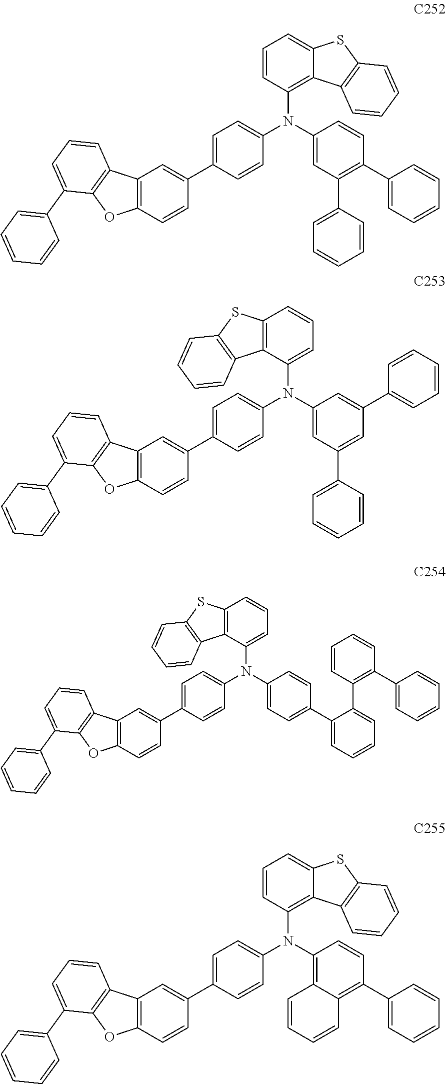 [SMILES: c1ccc(-c2ccc(N(c3ccc(-c4ccc5oc6c(-c7ccccc7)cccc6c5c4)cc3)c3cccc4sc5ccccc5c34)cc2-c2ccccc2)cc1 ; c1ccc(-c2cc(-c3ccccc3)cc(N(c3ccc(-c4ccc5oc6c(-c7ccccc7)cccc6c5c4)cc3)c3cccc4sc5ccccc5c34)c2)cc1 ; c1ccc(-c2ccccc2-c2ccccc2-c2ccc(N(c3ccc(-c4ccc5oc6c(-c7ccccc7)cccc6c5c4)cc3)c3cccc4sc5ccccc5c34)cc2)cc1 ; c1ccc(-c2ccc(N(c3ccc(-c4ccc5oc6c(-c7ccccc7)cccc6c5c4)cc3)c3cccc4sc5ccccc5c34)c3ccccc23)cc1] 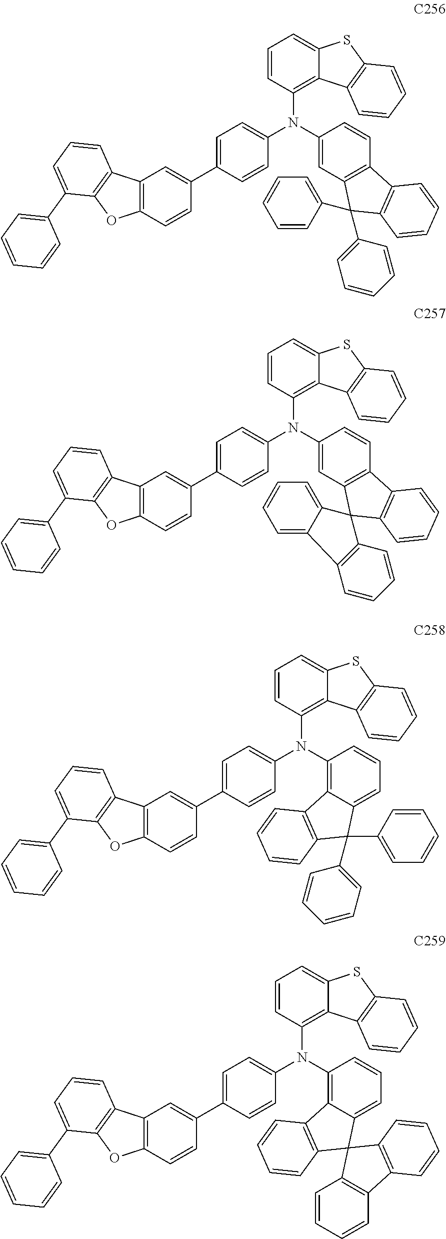 [SMILES: c1ccc(-c2cccc3c2oc2ccc(-c4ccc(N(c5ccc6c(c5)C(c5ccccc5)(c5ccccc5)c5ccccc5-6)c5cccc6sc7ccccc7c56)cc4)cc23)cc1 ; c1ccc(-c2cccc3c2oc2ccc(-c4ccc(N(c5ccc6c(c5)C5(c7ccccc7-c7ccccc75)c5ccccc5-6)c5cccc6sc7ccccc7c56)cc4)cc23)cc1 ; c1ccc(-c2cccc3c2oc2ccc(-c4ccc(N(c5cccc6c5-c5ccccc5C6(c5ccccc5)c5ccccc5)c5cccc6sc7ccccc7c56)cc4)cc23)cc1 ; c1ccc(-c2cccc3c2oc2ccc(-c4ccc(N(c5cccc6c5-c5ccccc5C65c6ccccc6-c6ccccc65)c5cccc6sc7ccccc7c56)cc4)cc23)cc1] 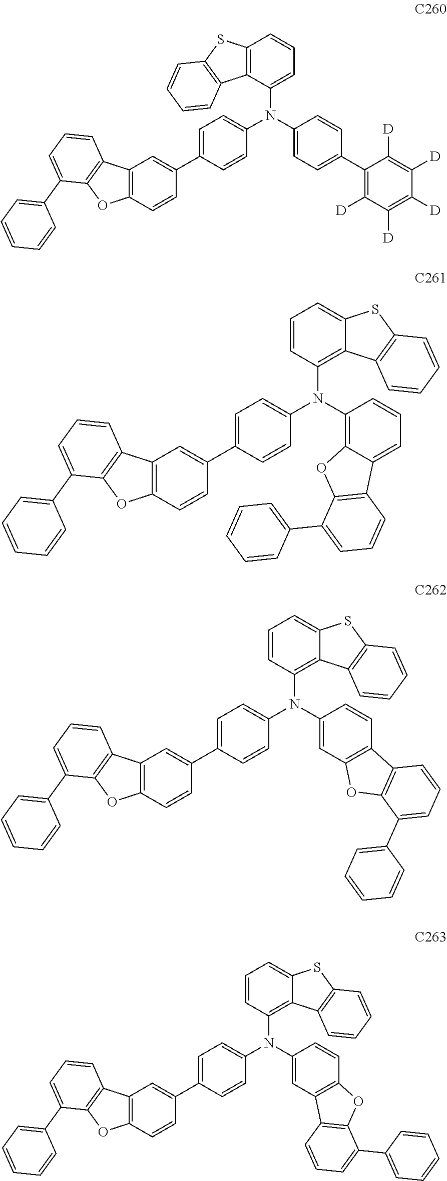 [SMILES: [2H]c1c([2H])c([2H])c(-c2ccc(N(c3ccc(-c4ccc5oc6c(-c7ccccc7)cccc6c5c4)cc3)c3cccc4sc5ccccc5c34)cc2)c([2H])c1[2H] ; c1ccc(-c2cccc3c2oc2ccc(-c4ccc(N(c5cccc6c5oc5c(-c7ccccc7)cccc56)c5cccc6sc7ccccc7c56)cc4)cc23)cc1 ; c1ccc(-c2cccc3c2oc2cc(N(c4ccc(-c5ccc6oc7c(-c8ccccc8)cccc7c6c5)cc4)c4cccc5sc6ccccc6c45)ccc23)cc1 ; c1ccc(-c2cccc3c2oc2ccc(-c4ccc(N(c5ccc6oc7c(-c8ccccc8)cccc7c6c5)c5cccc6sc7ccccc7c56)cc4)cc23)cc1] 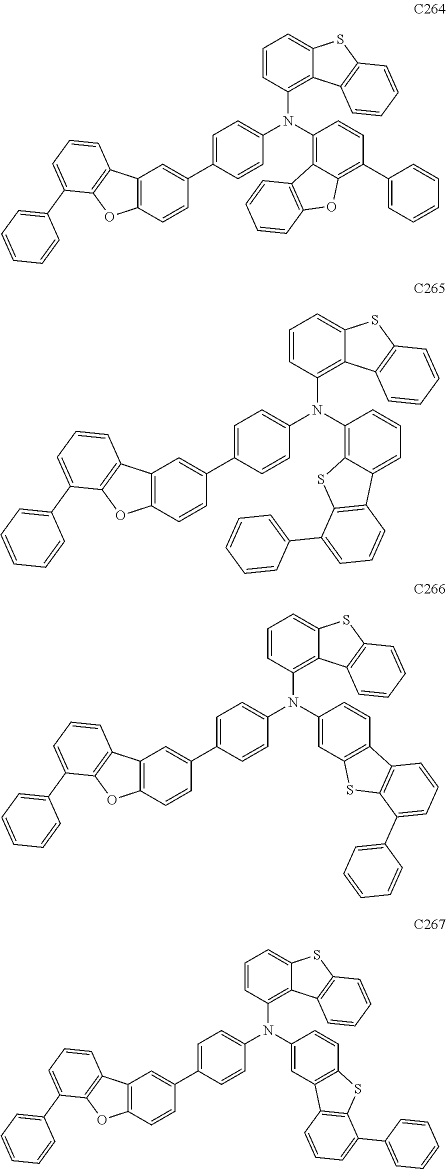 [SMILES: c1ccc(-c2cccc3c2oc2ccc(-c4ccc(N(c5cccc6sc7ccccc7c56)c5ccc(-c6ccccc6)c6oc7ccccc7c56)cc4)cc23)cc1 ; c1ccc(-c2cccc3c2oc2ccc(-c4ccc(N(c5cccc6c5sc5c(-c7ccccc7)cccc56)c5cccc6sc7ccccc7c56)cc4)cc23)cc1 ; c1ccc(-c2cccc3c2oc2ccc(-c4ccc(N(c5ccc6c(c5)sc5c(-c7ccccc7)cccc56)c5cccc6sc7ccccc7c56)cc4)cc23)cc1 ; c1ccc(-c2cccc3c2oc2ccc(-c4ccc(N(c5ccc6sc7c(-c8ccccc8)cccc7c6c5)c5cccc6sc7ccccc7c56)cc4)cc23)cc1] 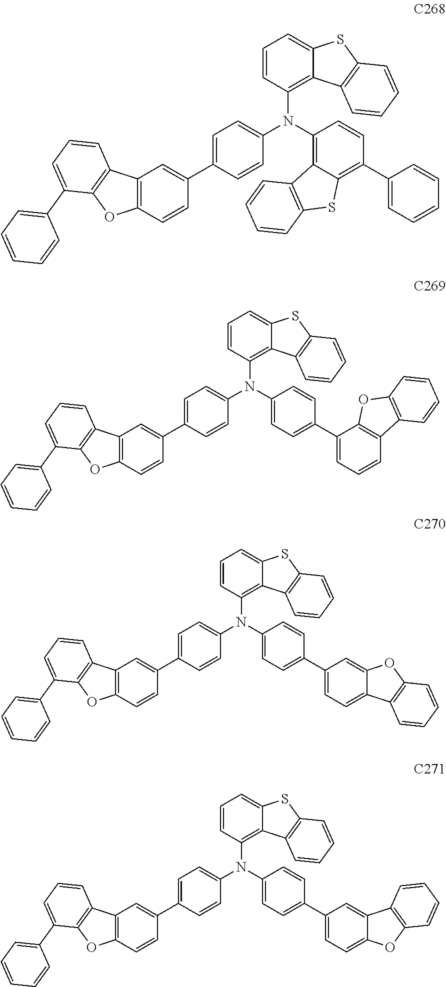 [SMILES: c1ccc(-c2cccc3c2oc2ccc(-c4ccc(N(c5cccc6sc7ccccc7c56)c5ccc(-c6ccccc6)c6sc7ccccc7c56)cc4)cc23)cc1 ; c1ccc(-c2cccc3c2oc2ccc(-c4ccc(N(c5ccc(-c6cccc7c6oc6ccccc67)cc5)c5cccc6sc7ccccc7c56)cc4)cc23)cc1 ; c1ccc(-c2cccc3c2oc2ccc(-c4ccc(N(c5ccc(-c6ccc7c(c6)oc6ccccc67)cc5)c5cccc6sc7ccccc7c56)cc4)cc23)cc1 ; c1ccc(-c2cccc3c2oc2ccc(-c4ccc(N(c5ccc(-c6ccc7oc8ccccc8c7c6)cc5)c5cccc6sc7ccccc7c56)cc4)cc23)cc1] 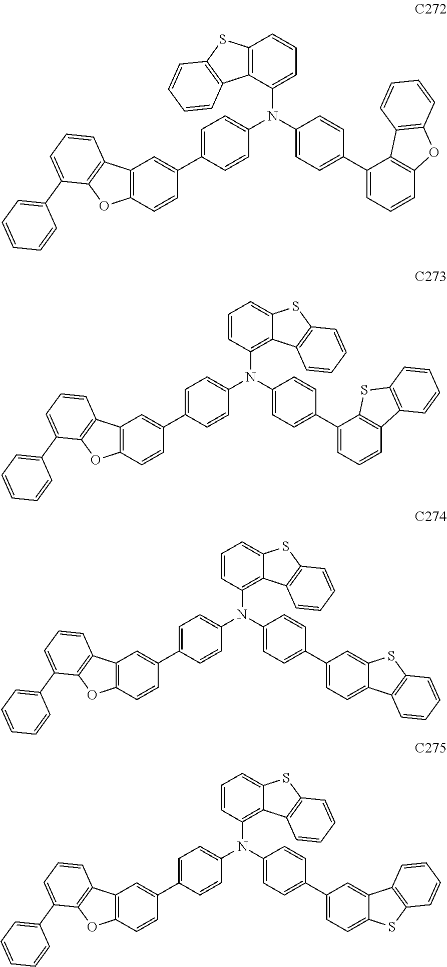 [SMILES: c1ccc(-c2cccc3c2oc2ccc(-c4ccc(N(c5ccc(-c6cccc7oc8ccccc8c67)cc5)c5cccc6sc7ccccc7c56)cc4)cc23)cc1 ; c1ccc(-c2cccc3c2oc2ccc(-c4ccc(N(c5ccc(-c6cccc7c6sc6ccccc67)cc5)c5cccc6sc7ccccc7c56)cc4)cc23)cc1 ; c1ccc(-c2cccc3c2oc2ccc(-c4ccc(N(c5ccc(-c6ccc7c(c6)sc6ccccc67)cc5)c5cccc6sc7ccccc7c56)cc4)cc23)cc1 ; c1ccc(-c2cccc3c2oc2ccc(-c4ccc(N(c5ccc(-c6ccc7sc8ccccc8c7c6)cc5)c5cccc6sc7ccccc7c56)cc4)cc23)cc1] 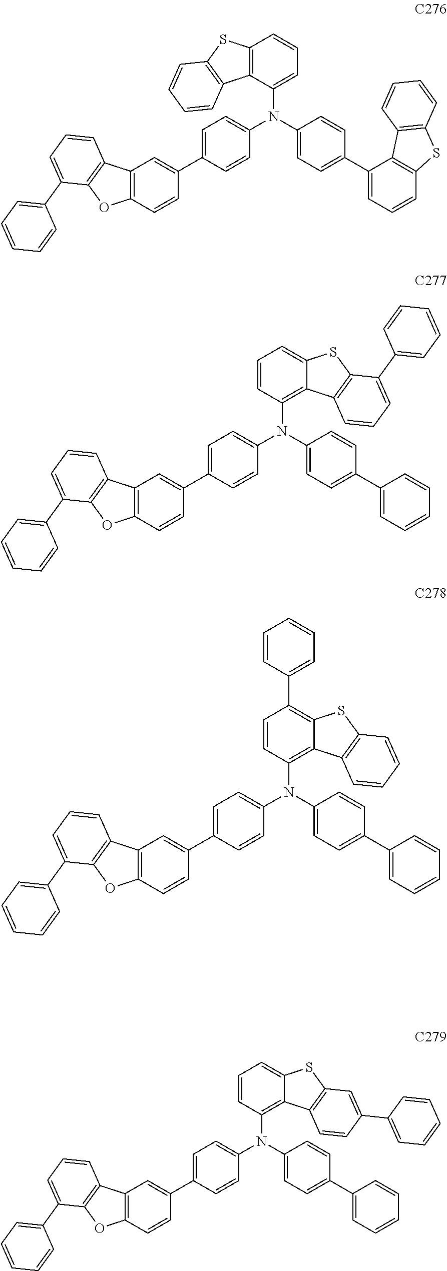 [SMILES: c1ccc(-c2cccc3c2oc2ccc(-c4ccc(N(c5ccc(-c6cccc7sc8ccccc8c67)cc5)c5cccc6sc7ccccc7c56)cc4)cc23)cc1 ; c1ccc(-c2ccc(N(c3ccc(-c4ccc5oc6c(-c7ccccc7)cccc6c5c4)cc3)c3cccc4sc5c(-c6ccccc6)cccc5c34)cc2)cc1 ; c1ccc(-c2ccc(N(c3ccc(-c4ccc5oc6c(-c7ccccc7)cccc6c5c4)cc3)c3ccc(-c4ccccc4)c4sc5ccccc5c34)cc2)cc1 ; c1ccc(-c2ccc(N(c3ccc(-c4ccc5oc6c(-c7ccccc7)cccc6c5c4)cc3)c3cccc4sc5cc(-c6ccccc6)ccc5c34)cc2)cc1] 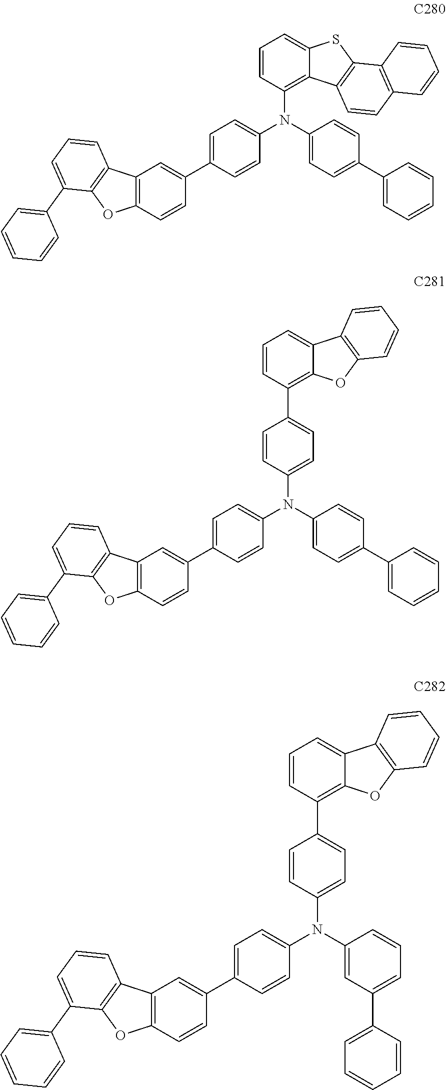 [SMILES: c1ccc(-c2ccc(N(c3ccc(-c4ccc5oc6c(-c7ccccc7)cccc6c5c4)cc3)c3cccc4sc5c6ccccc6ccc5c34)cc2)cc1 ; c1ccc(-c2ccc(N(c3ccc(-c4ccc5oc6c(-c7ccccc7)cccc6c5c4)cc3)c3ccc(-c4cccc5c4oc4ccccc45)cc3)cc2)cc1 ; c1ccc(-c2cccc(N(c3ccc(-c4ccc5oc6c(-c7ccccc7)cccc6c5c4)cc3)c3ccc(-c4cccc5c4oc4ccccc45)cc3)c2)cc1] 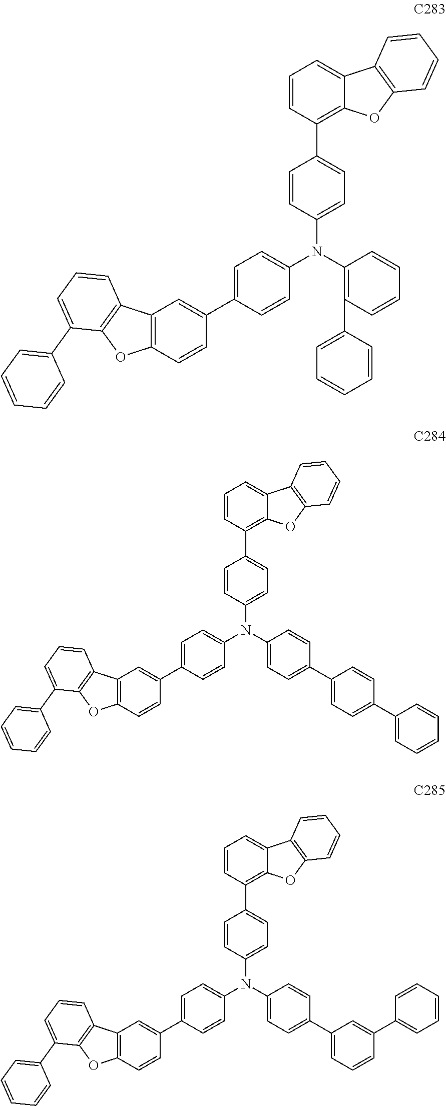 [SMILES: c1ccc(-c2ccccc2N(c2ccc(-c3ccc4oc5c(-c6ccccc6)cccc5c4c3)cc2)c2ccc(-c3cccc4c3oc3ccccc34)cc2)cc1 ; c1ccc(-c2ccc(-c3ccc(N(c4ccc(-c5ccc6oc7c(-c8ccccc8)cccc7c6c5)cc4)c4ccc(-c5cccc6c5oc5ccccc56)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6c5oc5ccccc56)cc4)cc3)c2)cc1] 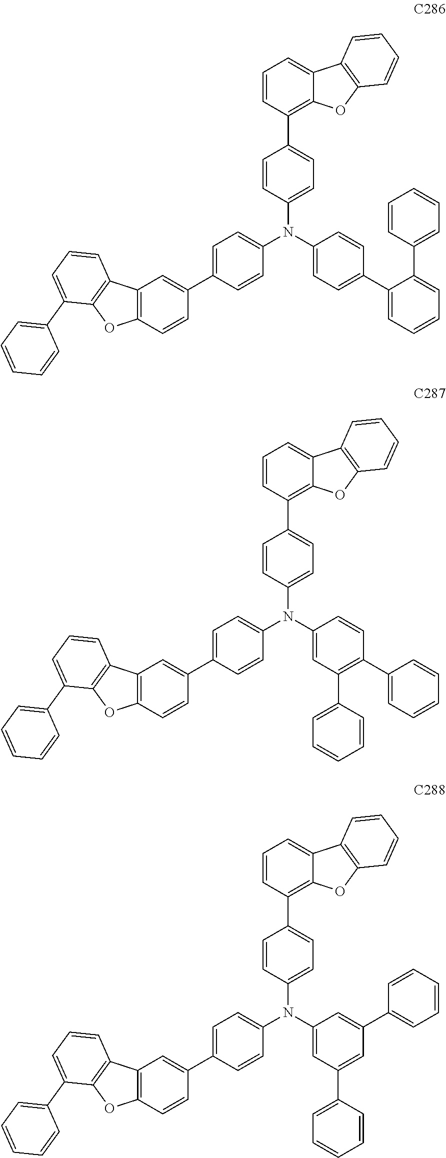 [SMILES: c1ccc(-c2ccccc2-c2ccc(N(c3ccc(-c4ccc5oc6c(-c7ccccc7)cccc6c5c4)cc3)c3ccc(-c4cccc5c4oc4ccccc45)cc3)cc2)cc1 ; c1ccc(-c2ccc(N(c3ccc(-c4ccc5oc6c(-c7ccccc7)cccc6c5c4)cc3)c3ccc(-c4cccc5c4oc4ccccc45)cc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc(-c4cccc5c4oc4ccccc45)cc3)c2)cc1] 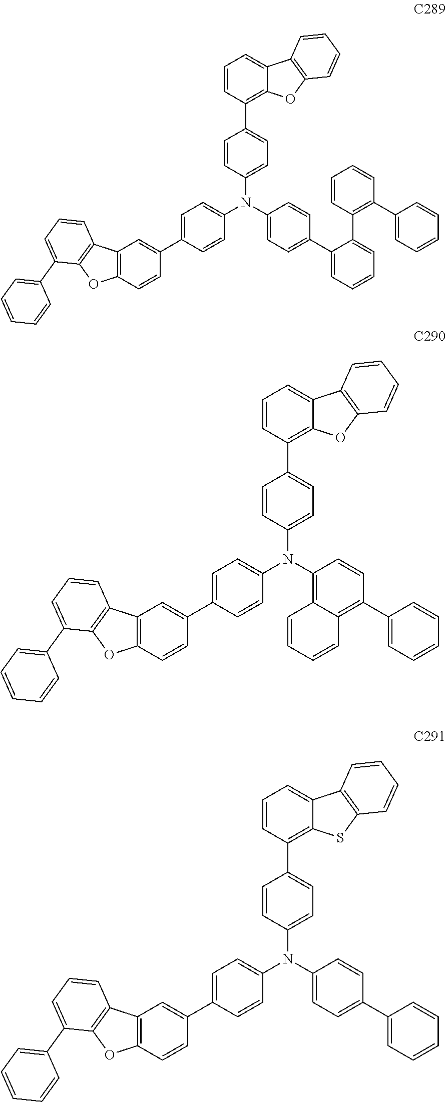 [SMILES: c1ccc(-c2ccccc2-c2ccccc2-c2ccc(N(c3ccc(-c4ccc5oc6c(-c7ccccc7)cccc6c5c4)cc3)c3ccc(-c4cccc5c4oc4ccccc45)cc3)cc2)cc1 ; c1ccc(-c2ccc(N(c3ccc(-c4ccc5oc6c(-c7ccccc7)cccc6c5c4)cc3)c3ccc(-c4cccc5c4oc4ccccc45)cc3)c3ccccc23)cc1 ; c1ccc(-c2ccc(N(c3ccc(-c4ccc5oc6c(-c7ccccc7)cccc6c5c4)cc3)c3ccc(-c4cccc5c4sc4ccccc45)cc3)cc2)cc1] 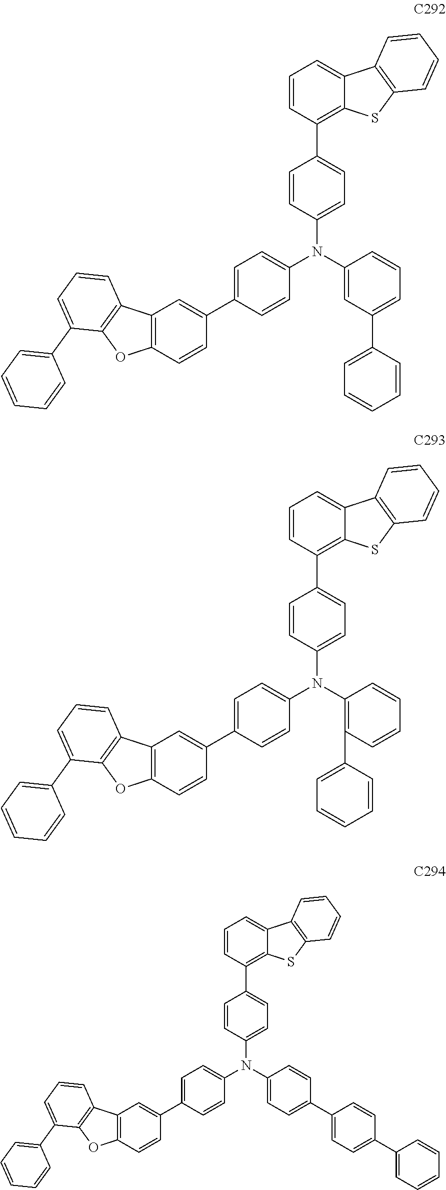 [SMILES: c1ccc(-c2cccc(N(c3ccc(-c4ccc5oc6c(-c7ccccc7)cccc6c5c4)cc3)c3ccc(-c4cccc5c4sc4ccccc45)cc3)c2)cc1 ; c1ccc(-c2ccccc2N(c2ccc(-c3ccc4oc5c(-c6ccccc6)cccc5c4c3)cc2)c2ccc(-c3cccc4c3sc3ccccc34)cc2)cc1 ; c1ccc(-c2ccc(-c3ccc(N(c4ccc(-c5ccc6oc7c(-c8ccccc8)cccc7c6c5)cc4)c4ccc(-c5cccc6c5sc5ccccc56)cc4)cc3)cc2)cc1] 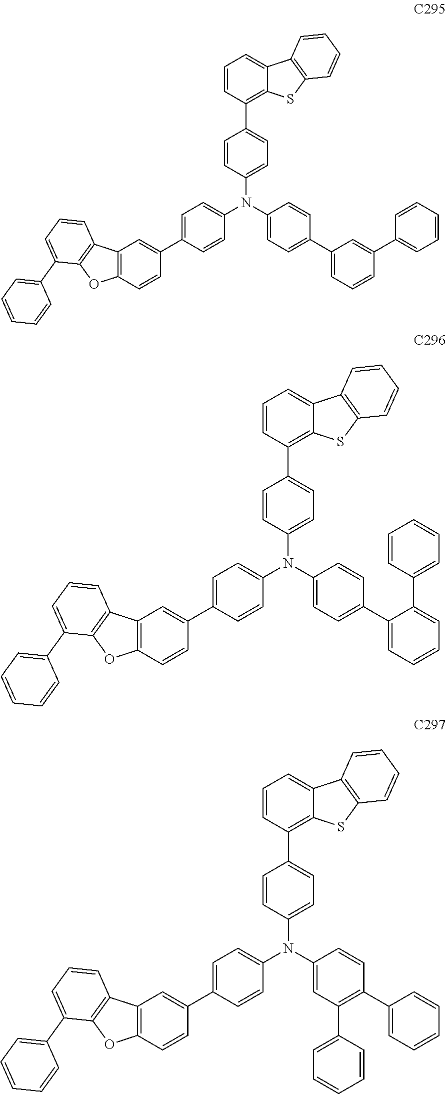 [SMILES: c1ccc(-c2cccc(-c3ccc(N(c4ccc(-c5ccc6oc7c(-c8ccccc8)cccc7c6c5)cc4)c4ccc(-c5cccc6c5sc5ccccc56)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4cccc5c4sc4ccccc45)cc3)cc2)cc1 ; c1ccc(-c2ccc(N(c3ccc(-c4ccc5oc6c(-c7ccccc7)cccc6c5c4)cc3)c3ccc(-c4cccc5c4sc4ccccc45)cc3)cc2-c2ccccc2)cc1] 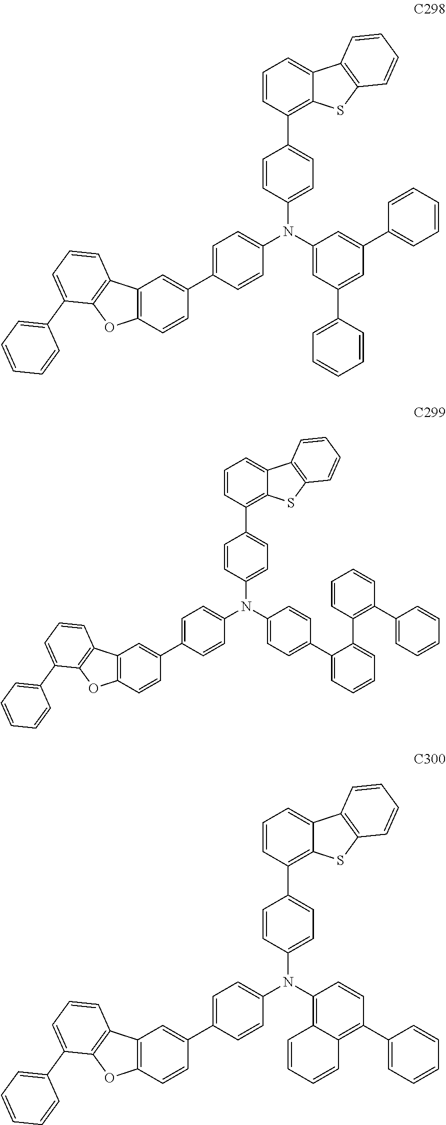 [SMILES: c1ccc(-c2cc(-c3ccccc3)cc(N(c3ccc(-c4ccc5oc6c(-c7ccccc7)cccc6c5c4)cc3)c3ccc(-c4cccc5c4sc4ccccc45)cc3)c2)cc1 ; c1ccc(-c2ccccc2-c2ccccc2-c2ccc(N(c3ccc(-c4ccc5oc6c(-c7ccccc7)cccc6c5c4)cc3)c3ccc(-c4cccc5c4sc4ccccc45)cc3)cc2)cc1 ; c1ccc(-c2ccc(N(c3ccc(-c4ccc5oc6c(-c7ccccc7)cccc6c5c4)cc3)c3ccc(-c4cccc5c4sc4ccccc45)cc3)c3ccccc23)cc1] 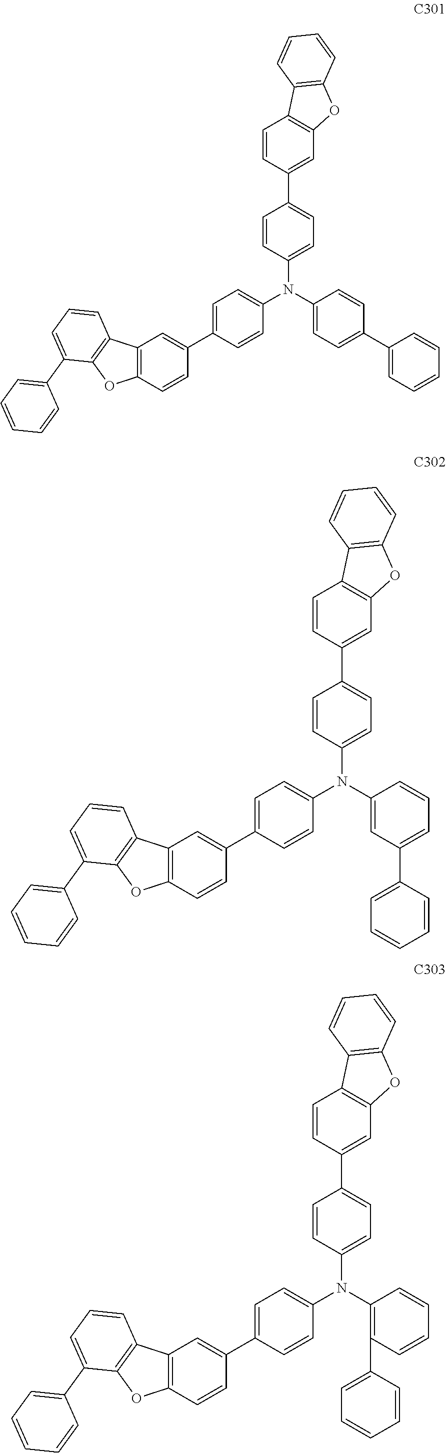 [SMILES: c1ccc(-c2ccc(N(c3ccc(-c4ccc5c(c4)oc4ccccc45)cc3)c3ccc(-c4ccc5oc6c(-c7ccccc7)cccc6c5c4)cc3)cc2)cc1 ; c1ccc(-c2cccc(N(c3ccc(-c4ccc5c(c4)oc4ccccc45)cc3)c3ccc(-c4ccc5oc6c(-c7ccccc7)cccc6c5c4)cc3)c2)cc1 ; c1ccc(-c2ccccc2N(c2ccc(-c3ccc4c(c3)oc3ccccc34)cc2)c2ccc(-c3ccc4oc5c(-c6ccccc6)cccc5c4c3)cc2)cc1] 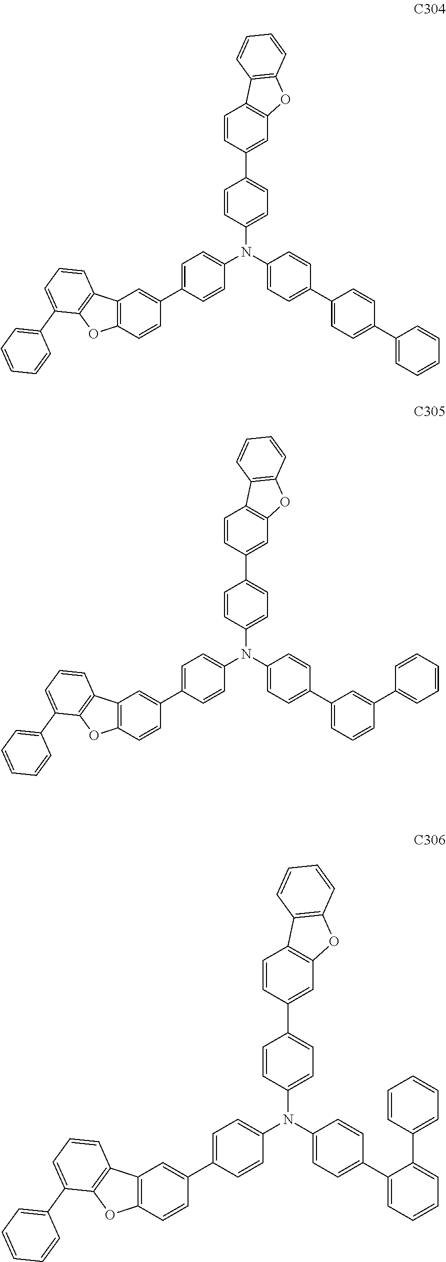 [SMILES: c1ccc(-c2ccc(-c3ccc(N(c4ccc(-c5ccc6c(c5)oc5ccccc56)cc4)c4ccc(-c5ccc6oc7c(-c8ccccc8)cccc7c6c5)cc4)cc3)cc2)cc1 ; c1ccc(-c2cccc(-c3ccc(N(c4ccc(-c5ccc6c(c5)oc5ccccc56)cc4)c4ccc(-c5ccc6oc7c(-c8ccccc8)cccc7c6c5)cc4)cc3)c2)cc1 ; c1ccc(-c2ccccc2-c2ccc(N(c3ccc(-c4ccc5c(c4)oc4ccccc45)cc3)c3ccc(-c4ccc5oc6c(-c7ccccc7)cccc6c5c4)cc3)cc2)cc1] 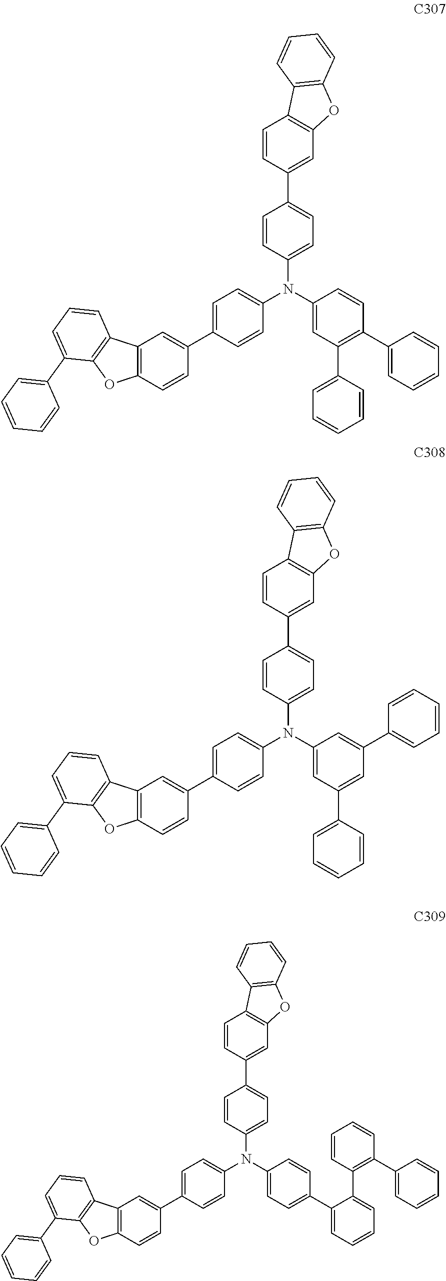 [SMILES: c1ccc(-c2ccc(N(c3ccc(-c4ccc5c(c4)oc4ccccc45)cc3)c3ccc(-c4ccc5oc6c(-c7ccccc7)cccc6c5c4)cc3)cc2-c2ccccc2)cc1 ; c1ccc(-c2cc(-c3ccccc3)cc(N(c3ccc(-c4ccc5c(c4)oc4ccccc45)cc3)c3ccc(-c4ccc5oc6c(-c7ccccc7)cccc6c5c4)cc3)c2)cc1 ; c1ccc(-c2ccccc2-c2ccccc2-c2ccc(N(c3ccc(-c4ccc5c(c4)oc4ccccc45)cc3)c3ccc(-c4ccc5oc6c(-c7ccccc7)cccc6c5c4)cc3)cc2)cc1] 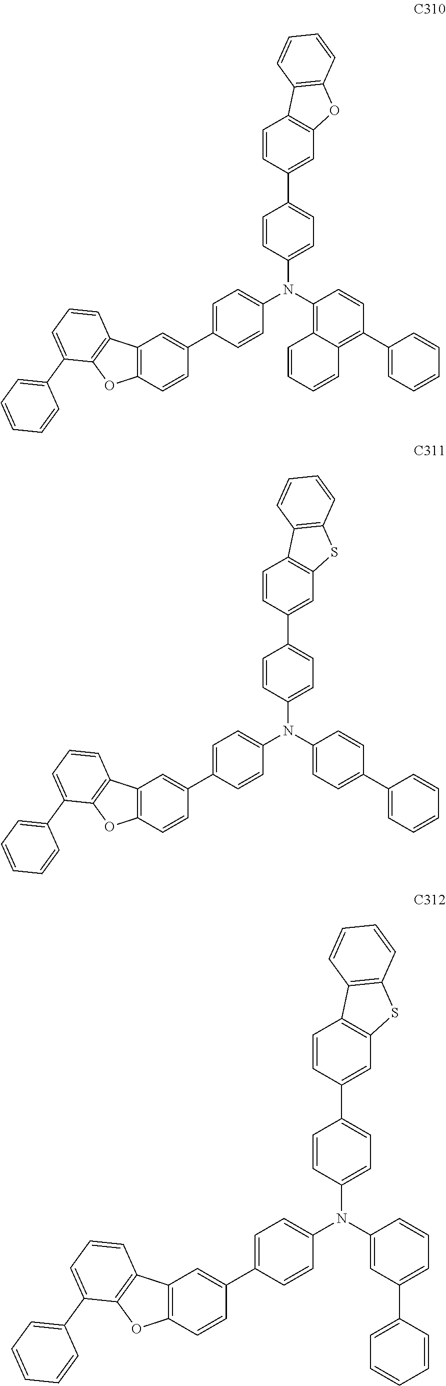 [SMILES: c1ccc(-c2ccc(N(c3ccc(-c4ccc5c(c4)oc4ccccc45)cc3)c3ccc(-c4ccc5oc6c(-c7ccccc7)cccc6c5c4)cc3)c3ccccc23)cc1 ; c1ccc(-c2ccc(N(c3ccc(-c4ccc5c(c4)sc4ccccc45)cc3)c3ccc(-c4ccc5oc6c(-c7ccccc7)cccc6c5c4)cc3)cc2)cc1 ; c1ccc(-c2cccc(N(c3ccc(-c4ccc5c(c4)sc4ccccc45)cc3)c3ccc(-c4ccc5oc6c(-c7ccccc7)cccc6c5c4)cc3)c2)cc1] 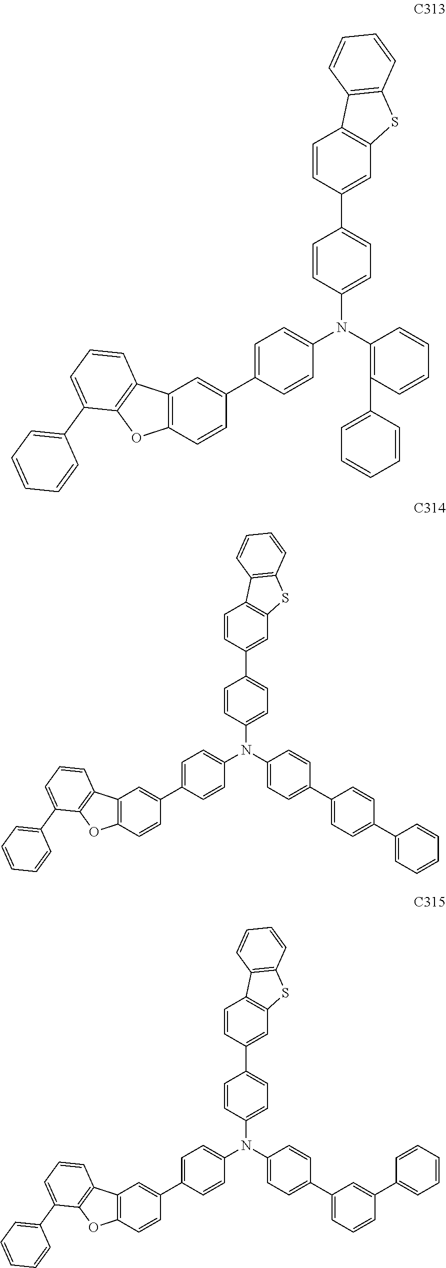 [SMILES: c1ccc(-c2ccccc2N(c2ccc(-c3ccc4c(c3)sc3ccccc34)cc2)c2ccc(-c3ccc4oc5c(-c6ccccc6)cccc5c4c3)cc2)cc1 ; c1ccc(-c2ccc(-c3ccc(N(c4ccc(-c5ccc6c(c5)sc5ccccc56)cc4)c4ccc(-c5ccc6oc7c(-c8ccccc8)cccc7c6c5)cc4)cc3)cc2)cc1 ; c1ccc(-c2cccc(-c3ccc(N(c4ccc(-c5ccc6c(c5)sc5ccccc56)cc4)c4ccc(-c5ccc6oc7c(-c8ccccc8)cccc7c6c5)cc4)cc3)c2)cc1] 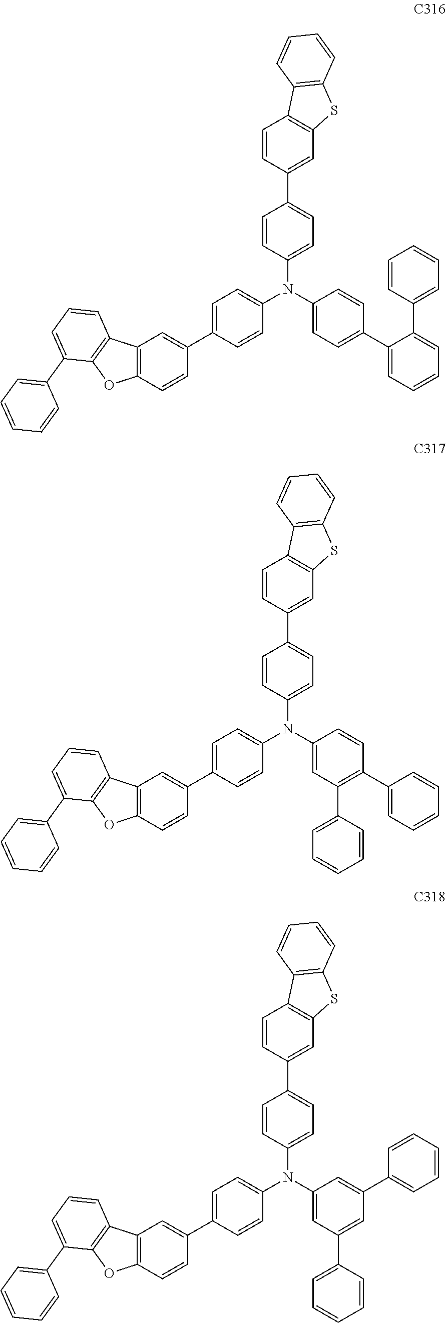 [SMILES: c1ccc(-c2ccccc2-c2ccc(N(c3ccc(-c4ccc5c(c4)sc4ccccc45)cc3)c3ccc(-c4ccc5oc6c(-c7ccccc7)cccc6c5c4)cc3)cc2)cc1 ; c1ccc(-c2ccc(N(c3ccc(-c4ccc5c(c4)sc4ccccc45)cc3)c3ccc(-c4ccc5oc6c(-c7ccccc7)cccc6c5c4)cc3)cc2-c2ccccc2)cc1 ; c1ccc(-c2cc(-c3ccccc3)cc(N(c3ccc(-c4ccc5c(c4)sc4ccccc45)cc3)c3ccc(-c4ccc5oc6c(-c7ccccc7)cccc6c5c4)cc3)c2)cc1] 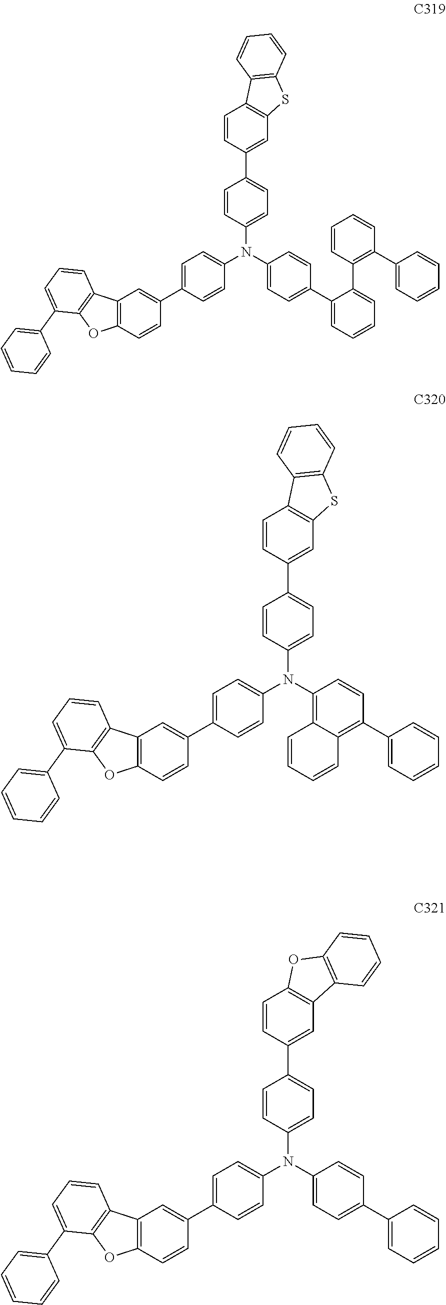 [SMILES: c1ccc(-c2ccccc2-c2ccccc2-c2ccc(N(c3ccc(-c4ccc5c(c4)sc4ccccc45)cc3)c3ccc(-c4ccc5oc6c(-c7ccccc7)cccc6c5c4)cc3)cc2)cc1 ; c1ccc(-c2ccc(N(c3ccc(-c4ccc5c(c4)sc4ccccc45)cc3)c3ccc(-c4ccc5oc6c(-c7ccccc7)cccc6c5c4)cc3)c3ccccc23)cc1 ; c1ccc(-c2ccc(N(c3ccc(-c4ccc5oc6ccccc6c5c4)cc3)c3ccc(-c4ccc5oc6c(-c7ccccc7)cccc6c5c4)cc3)cc2)cc1] 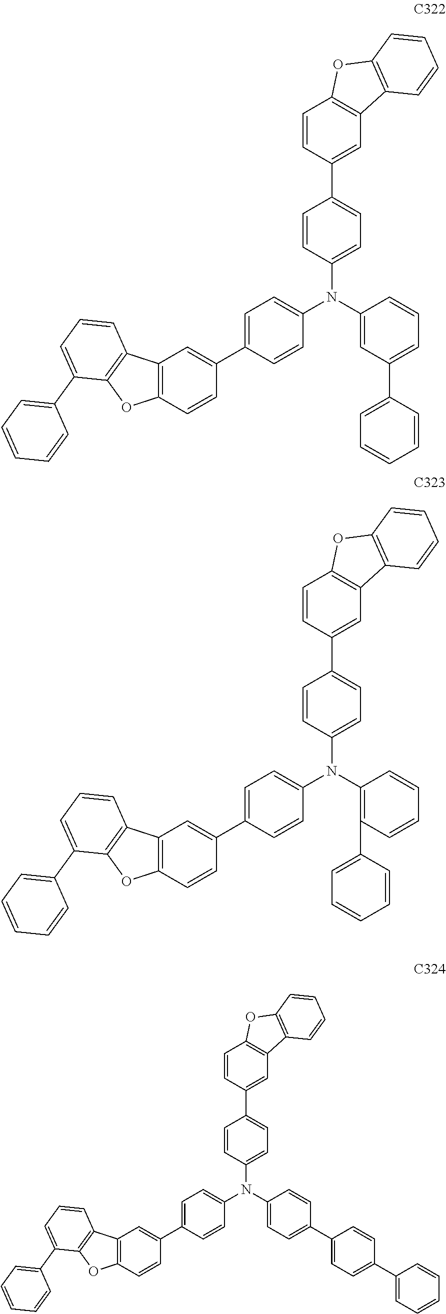 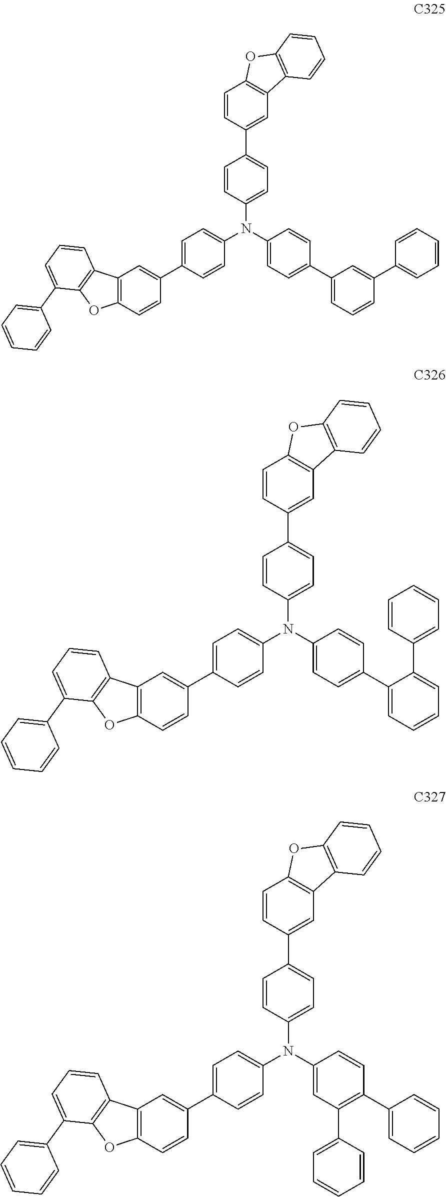 [SMILES: c1ccc(-c2cccc(-c3ccc(N(c4ccc(-c5ccc6oc7ccccc7c6c5)cc4)c4ccc(-c5ccc6oc7c(-c8ccccc8)cccc7c6c5)cc4)cc3)c2)cc1 ; c1ccc(-c2ccccc2-c2ccc(N(c3ccc(-c4ccc5oc6ccccc6c5c4)cc3)c3ccc(-c4ccc5oc6c(-c7ccccc7)cccc6c5c4)cc3)cc2)cc1 ; c1ccc(-c2ccc(N(c3ccc(-c4ccc5oc6ccccc6c5c4)cc3)c3ccc(-c4ccc5oc6c(-c7ccccc7)cccc6c5c4)cc3)cc2-c2ccccc2)cc1] 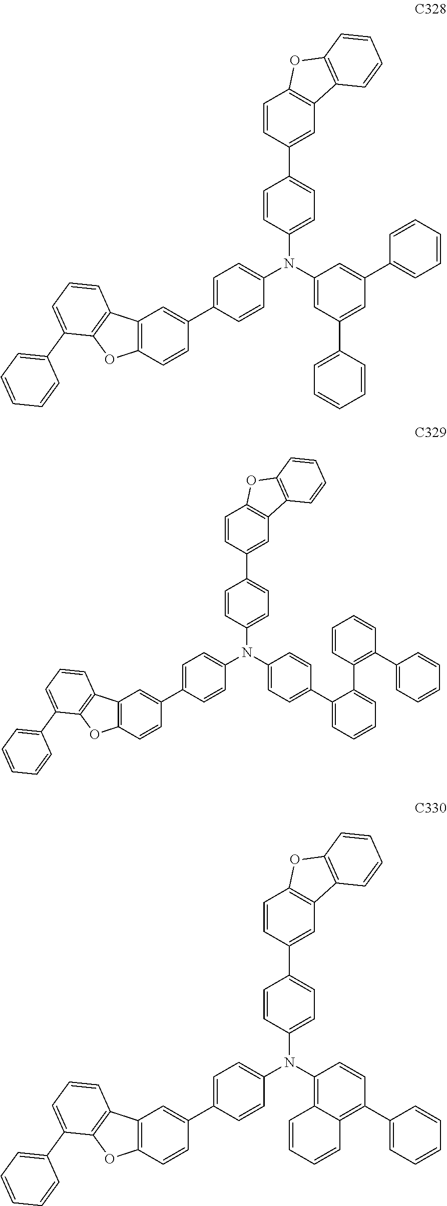 [SMILES: c1ccc(-c2cc(-c3ccccc3)cc(N(c3ccc(-c4ccc5oc6ccccc6c5c4)cc3)c3ccc(-c4ccc5oc6c(-c7ccccc7)cccc6c5c4)cc3)c2)cc1 ; c1ccc(-c2ccccc2-c2ccccc2-c2ccc(N(c3ccc(-c4ccc5oc6ccccc6c5c4)cc3)c3ccc(-c4ccc5oc6c(-c7ccccc7)cccc6c5c4)cc3)cc2)cc1 ; c1ccc(-c2ccc(N(c3ccc(-c4ccc5oc6ccccc6c5c4)cc3)c3ccc(-c4ccc5oc6c(-c7ccccc7)cccc6c5c4)cc3)c3ccccc23)cc1] 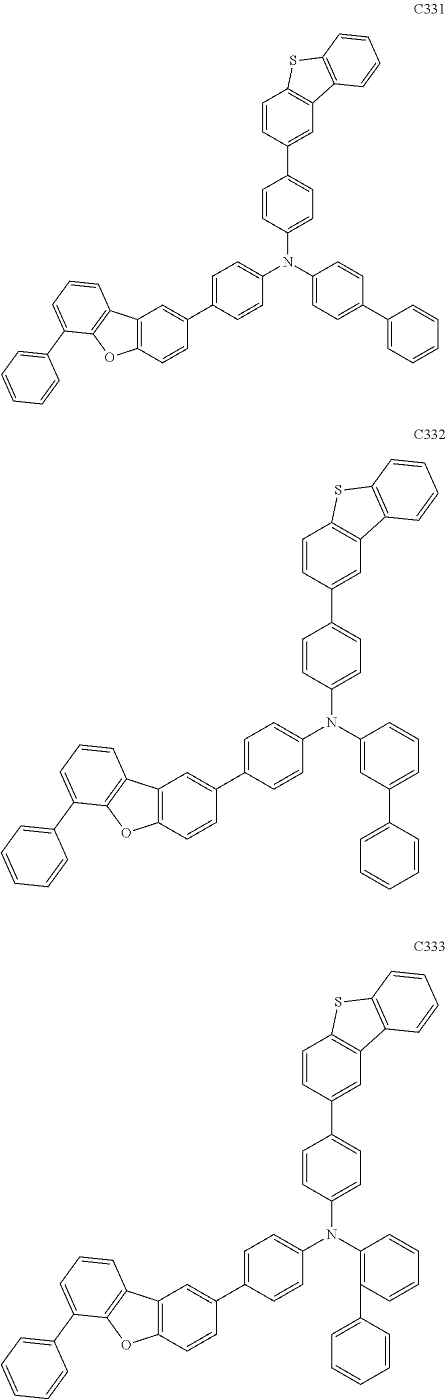 [SMILES: c1ccc(-c2ccc(N(c3ccc(-c4ccc5oc6c(-c7ccccc7)cccc6c5c4)cc3)c3ccc(-c4ccc5sc6ccccc6c5c4)cc3)cc2)cc1 ; c1ccc(-c2cccc(N(c3ccc(-c4ccc5oc6c(-c7ccccc7)cccc6c5c4)cc3)c3ccc(-c4ccc5sc6ccccc6c5c4)cc3)c2)cc1 ; c1ccc(-c2ccccc2N(c2ccc(-c3ccc4oc5c(-c6ccccc6)cccc5c4c3)cc2)c2ccc(-c3ccc4sc5ccccc5c4c3)cc2)cc1] 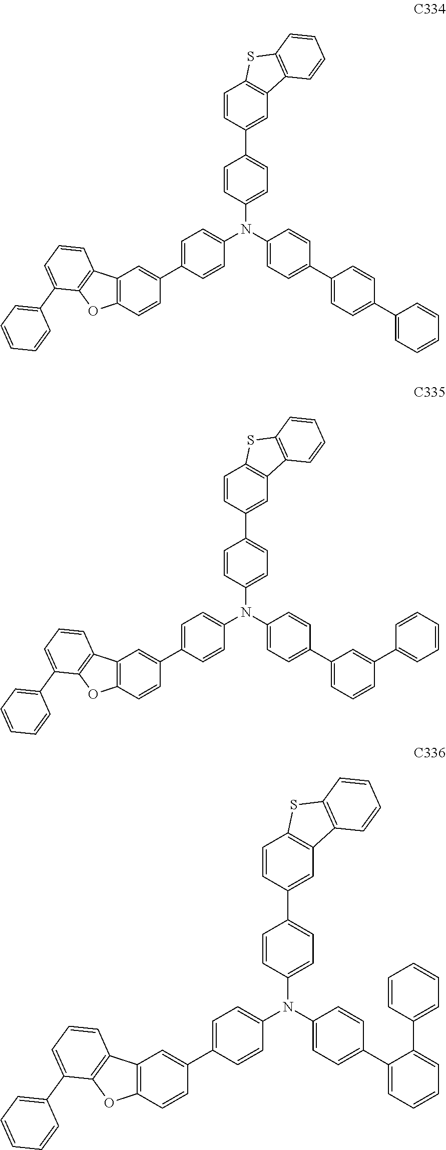 [SMILES: c1ccc(-c2ccc(-c3ccc(N(c4ccc(-c5ccc6oc7c(-c8ccccc8)cccc7c6c5)cc4)c4ccc(-c5ccc6sc7ccccc7c6c5)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5ccc6sc7ccccc7c6c5)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4ccc5sc6ccccc6c5c4)cc3)cc2)cc1] 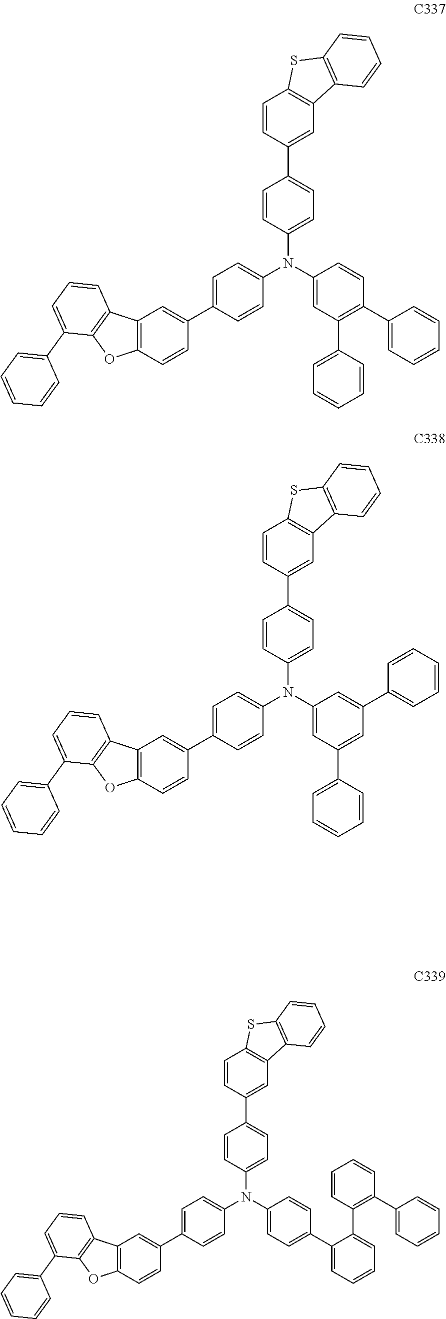 [SMILES: c1ccc(-c2ccc(N(c3ccc(-c4ccc5oc6c(-c7ccccc7)cccc6c5c4)cc3)c3ccc(-c4ccc5sc6ccccc6c5c4)cc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc(-c4ccc5sc6ccccc6c5c4)cc3)c2)cc1 ; c1ccc(-c2ccccc2-c2ccccc2-c2ccc(N(c3ccc(-c4ccc5oc6c(-c7ccccc7)cccc6c5c4)cc3)c3ccc(-c4ccc5sc6ccccc6c5c4)cc3)cc2)cc1] 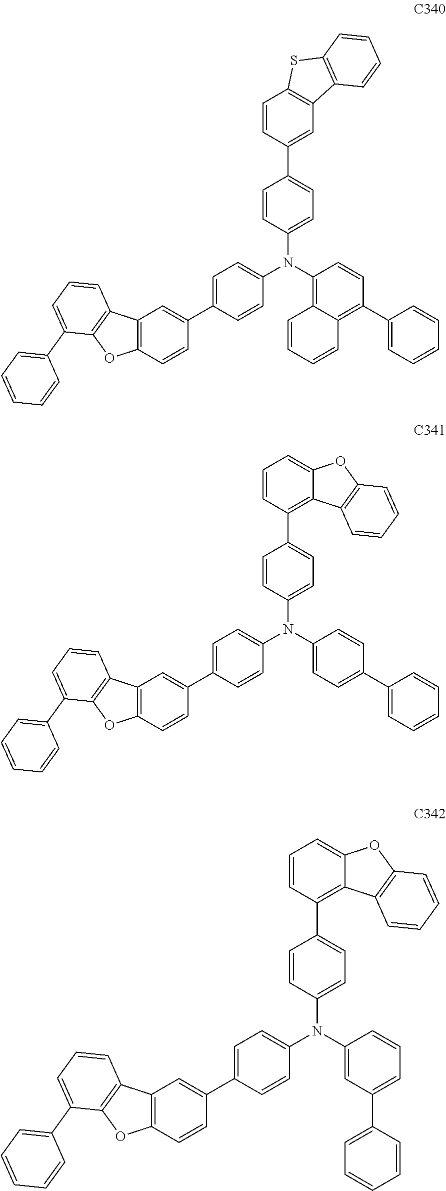 [SMILES: c1ccc(-c2ccc(N(c3ccc(-c4ccc5oc6c(-c7ccccc7)cccc6c5c4)cc3)c3ccc(-c4ccc5sc6ccccc6c5c4)cc3)c3ccccc23)cc1 ; c1ccc(-c2ccc(N(c3ccc(-c4ccc5oc6c(-c7ccccc7)cccc6c5c4)cc3)c3ccc(-c4cccc5oc6ccccc6c45)cc3)cc2)cc1 ; c1ccc(-c2cccc(N(c3ccc(-c4ccc5oc6c(-c7ccccc7)cccc6c5c4)cc3)c3ccc(-c4cccc5oc6ccccc6c45)cc3)c2)cc1] 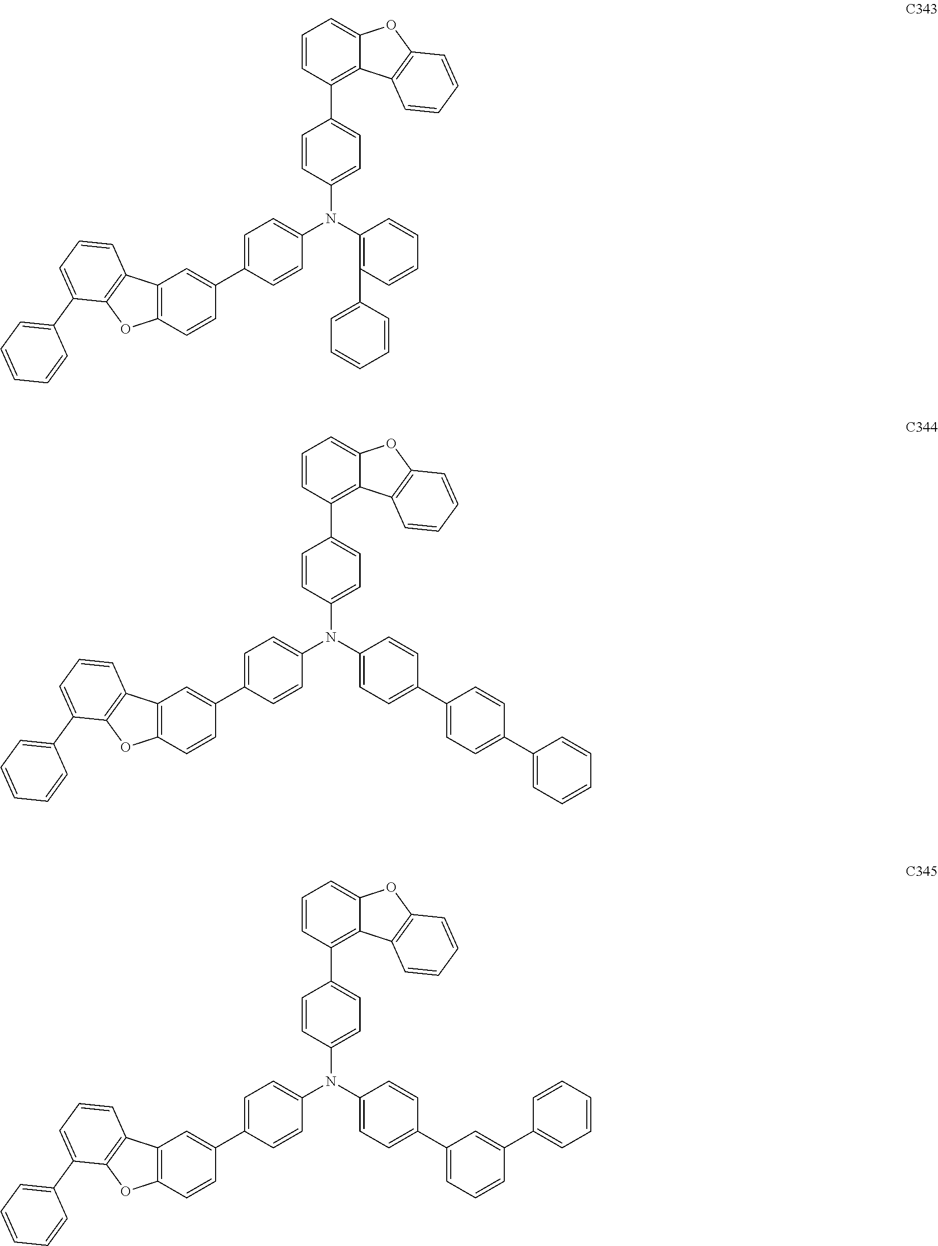 [SMILES: c1ccc(-c2ccccc2N(c2ccc(-c3ccc4oc5c(-c6ccccc6)cccc5c4c3)cc2)c2ccc(-c3cccc4oc5ccccc5c34)cc2)cc1 ; c1ccc(-c2ccc(-c3ccc(N(c4ccc(-c5ccc6oc7c(-c8ccccc8)cccc7c6c5)cc4)c4ccc(-c5cccc6oc7ccccc7c56)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6oc7ccccc7c56)cc4)cc3)c2)cc1] 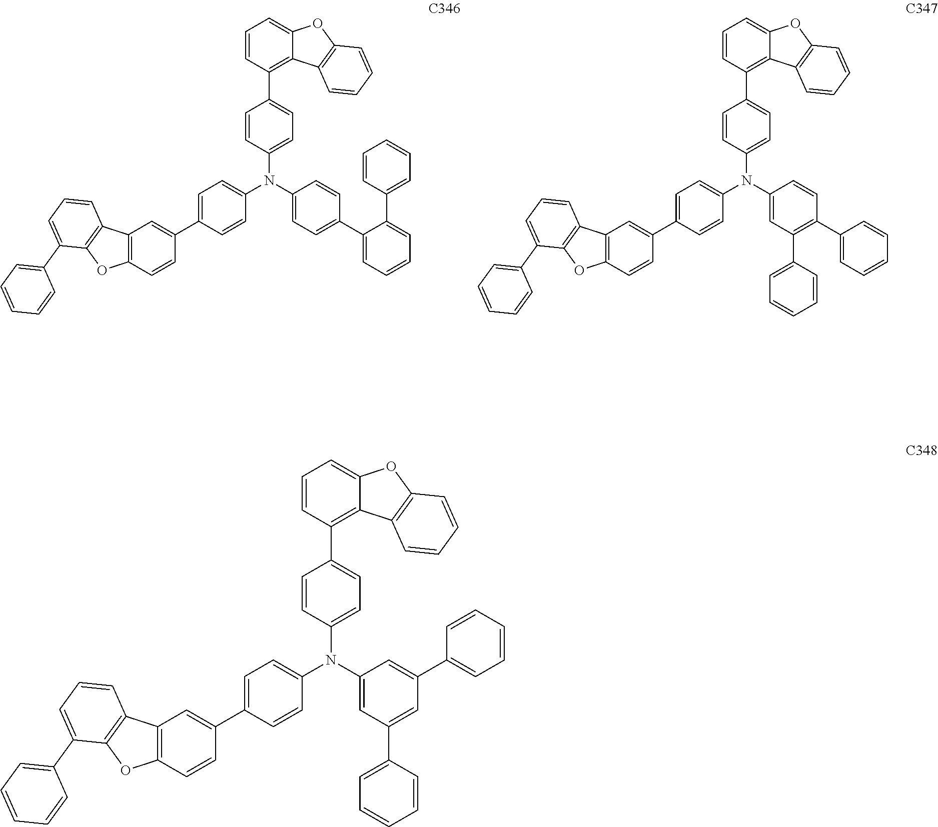 [SMILES: c1ccc(-c2ccccc2-c2ccc(N(c3ccc(-c4ccc5oc6c(-c7ccccc7)cccc6c5c4)cc3)c3ccc(-c4cccc5oc6ccccc6c45)cc3)cc2)cc1 ; c1ccc(-c2ccc(N(c3ccc(-c4ccc5oc6c(-c7ccccc7)cccc6c5c4)cc3)c3ccc(-c4cccc5oc6ccccc6c45)cc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc(-c4cccc5oc6ccccc6c45)cc3)c2)cc1] 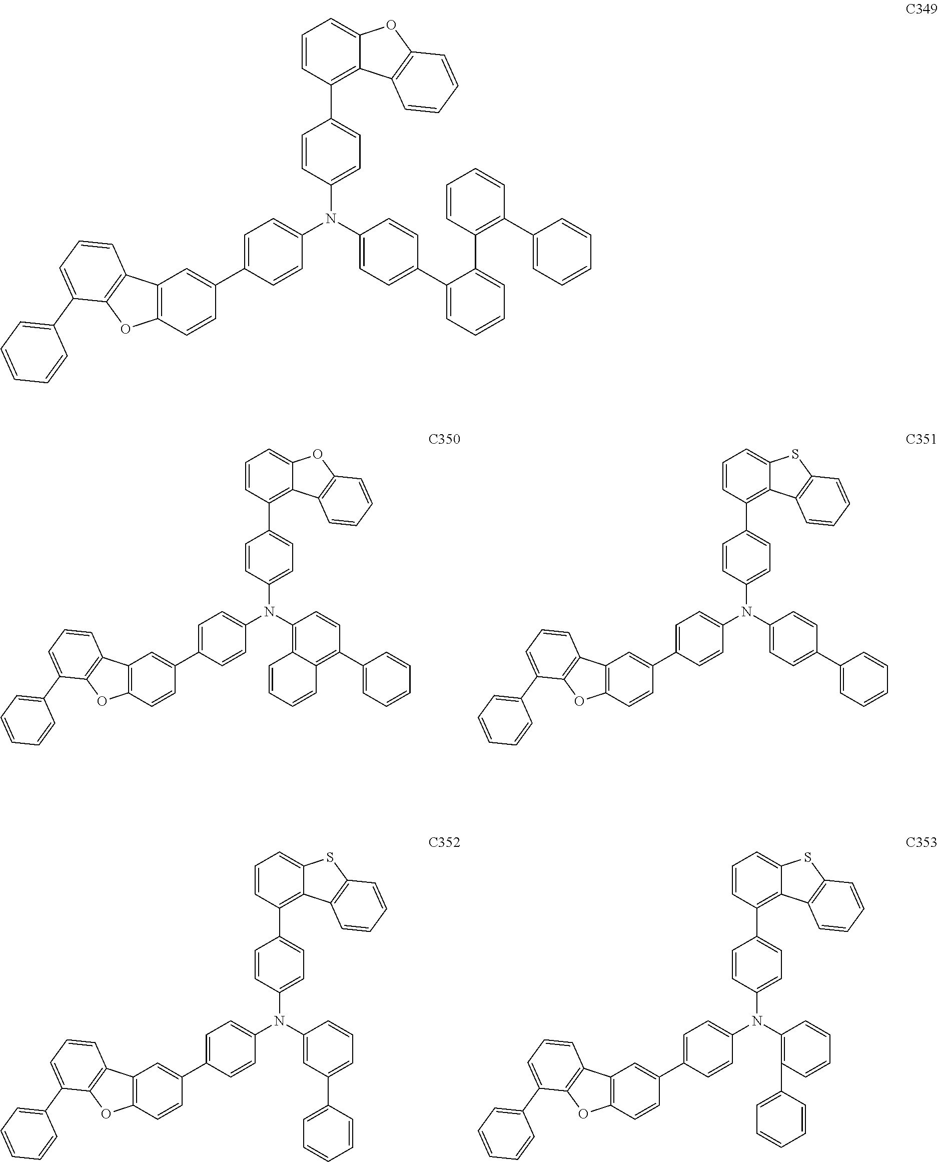 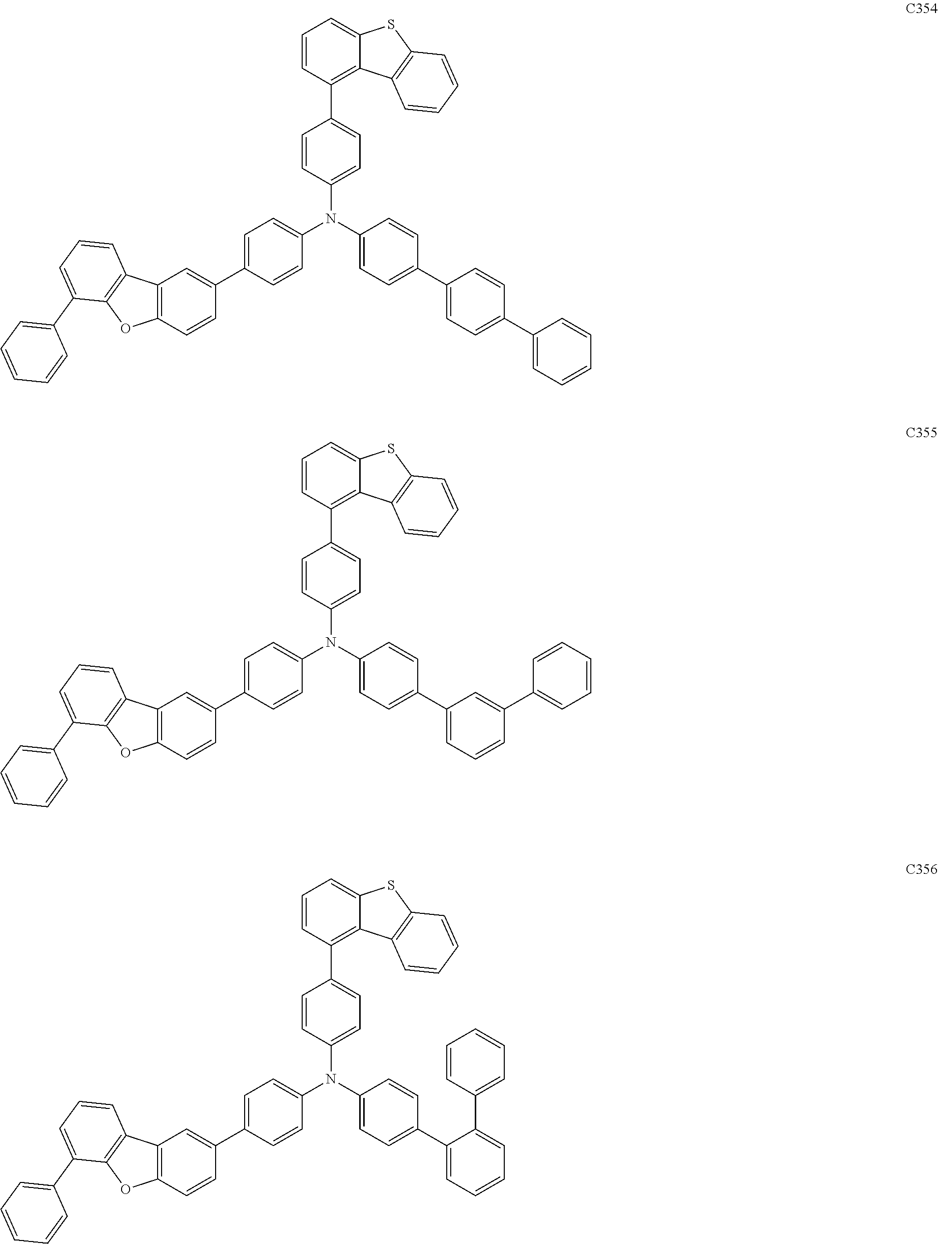 [SMILES: c1ccc(-c2ccc(-c3ccc(N(c4ccc(-c5ccc6oc7c(-c8ccccc8)cccc7c6c5)cc4)c4ccc(-c5cccc6sc7ccccc7c56)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6sc7ccccc7c56)cc4)cc3)c2)cc1 ; c1ccc(-c2ccccc2-c2ccc(N(c3ccc(-c4ccc5oc6c(-c7ccccc7)cccc6c5c4)cc3)c3ccc(-c4cccc5sc6ccccc6c45)cc3)cc2)cc1] 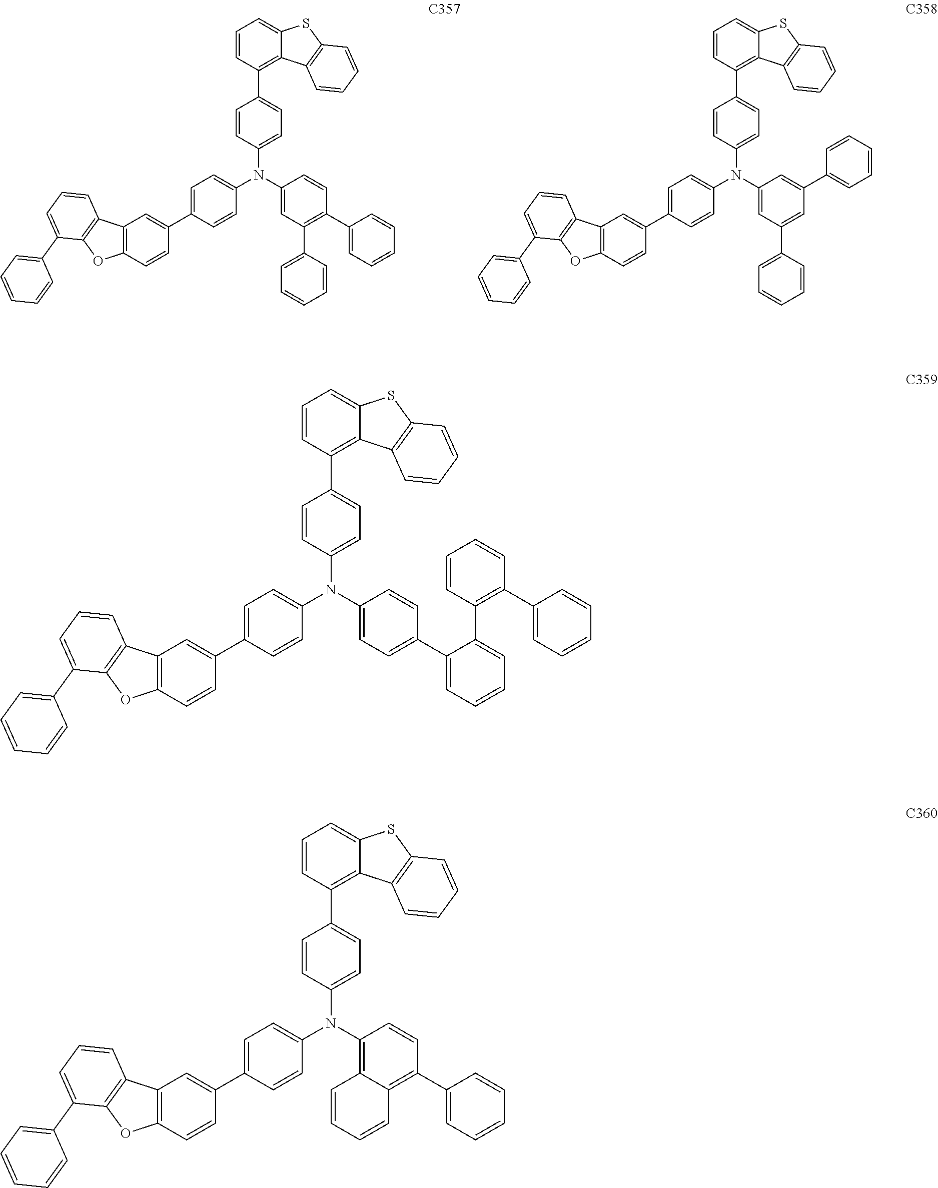 [SMILES: c1ccc(-c2ccc(N(c3ccc(-c4ccc5oc6c(-c7ccccc7)cccc6c5c4)cc3)c3ccc(-c4cccc5sc6ccccc6c45)cc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc(-c4cccc5sc6ccccc6c45)cc3)c2)cc1 ; c1ccc(-c2ccccc2-c2ccccc2-c2ccc(N(c3ccc(-c4ccc5oc6c(-c7ccccc7)cccc6c5c4)cc3)c3ccc(-c4cccc5sc6ccccc6c45)cc3)cc2)cc1 ; c1ccc(-c2ccc(N(c3ccc(-c4ccc5oc6c(-c7ccccc7)cccc6c5c4)cc3)c3ccc(-c4cccc5sc6ccccc6c45)cc3)c3ccccc23)cc1] 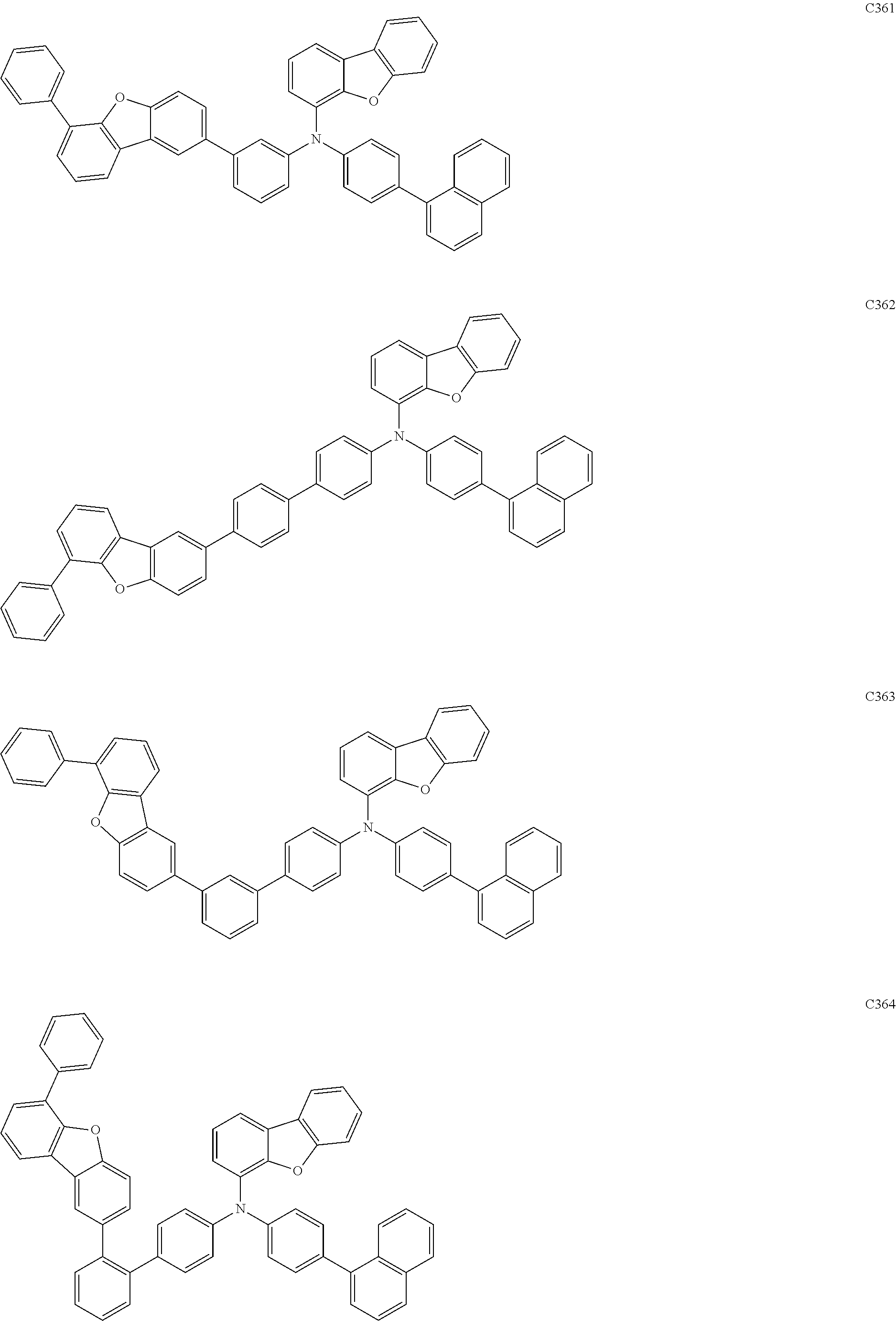 [SMILES: c1ccc(-c2cccc3c2oc2ccc(-c4cccc(N(c5ccc(-c6cccc7ccccc67)cc5)c5cccc6c5oc5ccccc56)c4)cc23)cc1 ; c1ccc(-c2cccc3c2oc2ccc(-c4ccc(-c5ccc(N(c6ccc(-c7cccc8ccccc78)cc6)c6cccc7c6oc6ccccc67)cc5)cc4)cc23)cc1 ; c1ccc(-c2cccc3c2oc2ccc(-c4cccc(-c5ccc(N(c6ccc(-c7cccc8ccccc78)cc6)c6cccc7c6oc6ccccc67)cc5)c4)cc23)cc1 ; c1ccc(-c2cccc3c2oc2ccc(-c4ccccc4-c4ccc(N(c5ccc(-c6cccc7ccccc67)cc5)c5cccc6c5oc5ccccc56)cc4)cc23)cc1] 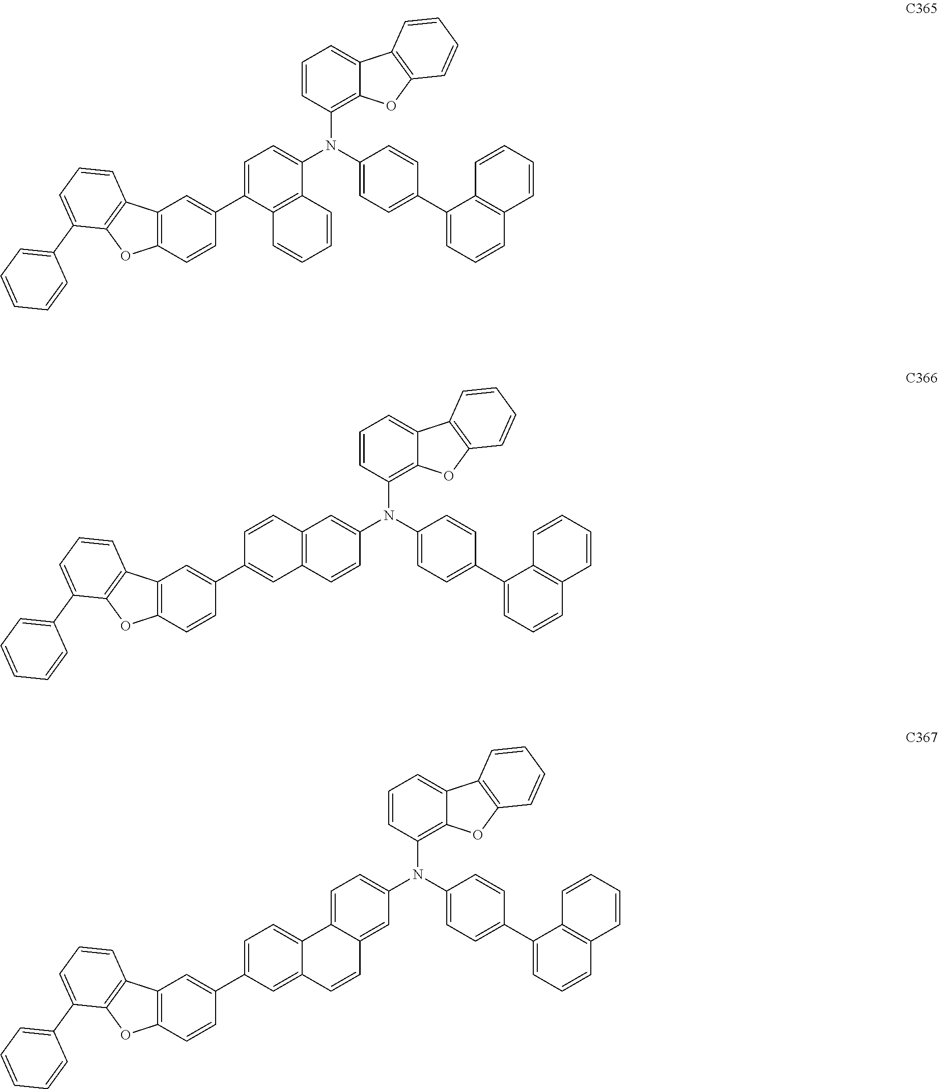 [SMILES: c1ccc(-c2cccc3c2oc2ccc(-c4ccc(N(c5ccc(-c6cccc7ccccc67)cc5)c5cccc6c5oc5ccccc56)c5ccccc45)cc23)cc1 ; c1ccc(-c2cccc3c2oc2ccc(-c4ccc5cc(N(c6ccc(-c7cccc8ccccc78)cc6)c6cccc7c6oc6ccccc67)ccc5c4)cc23)cc1 ; c1ccc(-c2cccc3c2oc2ccc(-c4ccc5c(ccc6cc(N(c7ccc(-c8cccc9ccccc89)cc7)c7cccc8c7oc7ccccc78)ccc65)c4)cc23)cc1] 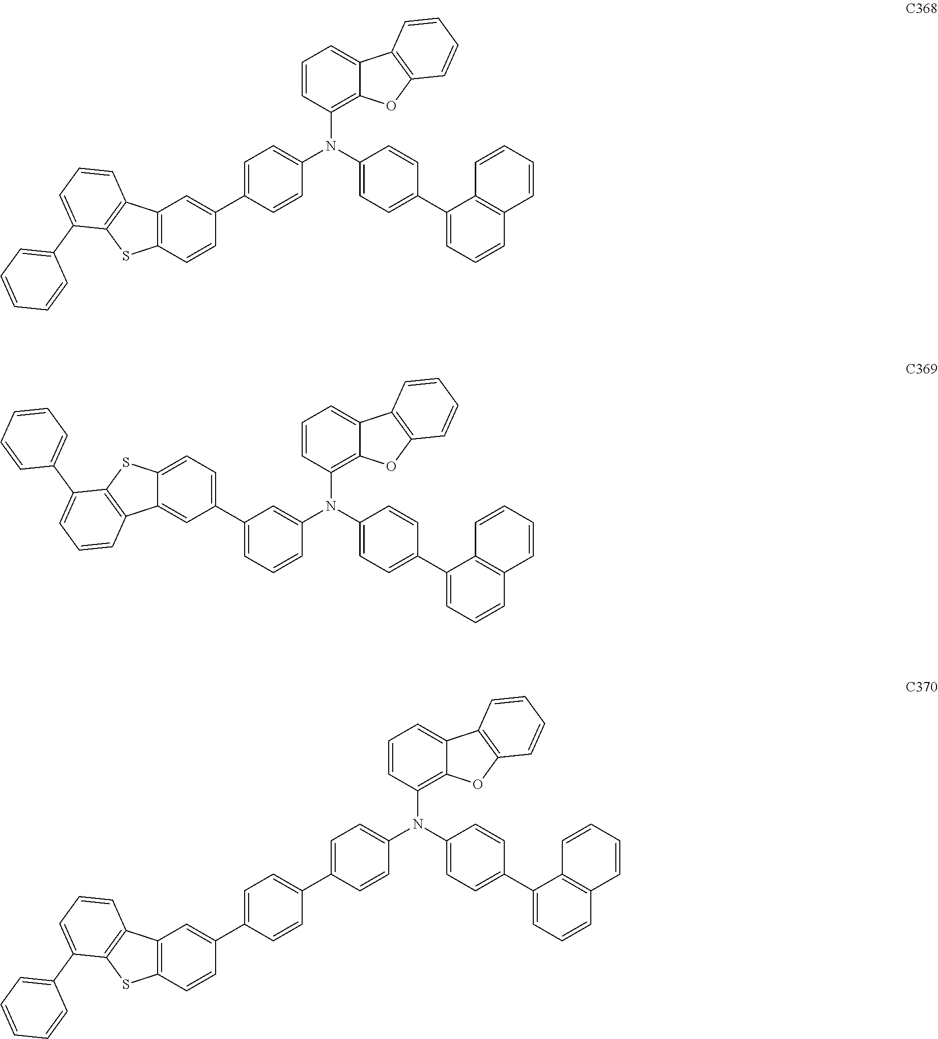 [SMILES: c1ccc(-c2cccc3c2sc2ccc(-c4ccc(N(c5ccc(-c6cccc7ccccc67)cc5)c5cccc6c5oc5ccccc56)cc4)cc23)cc1 ; c1ccc(-c2cccc3c2sc2ccc(-c4cccc(N(c5ccc(-c6cccc7ccccc67)cc5)c5cccc6c5oc5ccccc56)c4)cc23)cc1 ; c1ccc(-c2cccc3c2sc2ccc(-c4ccc(-c5ccc(N(c6ccc(-c7cccc8ccccc78)cc6)c6cccc7c6oc6ccccc67)cc5)cc4)cc23)cc1] 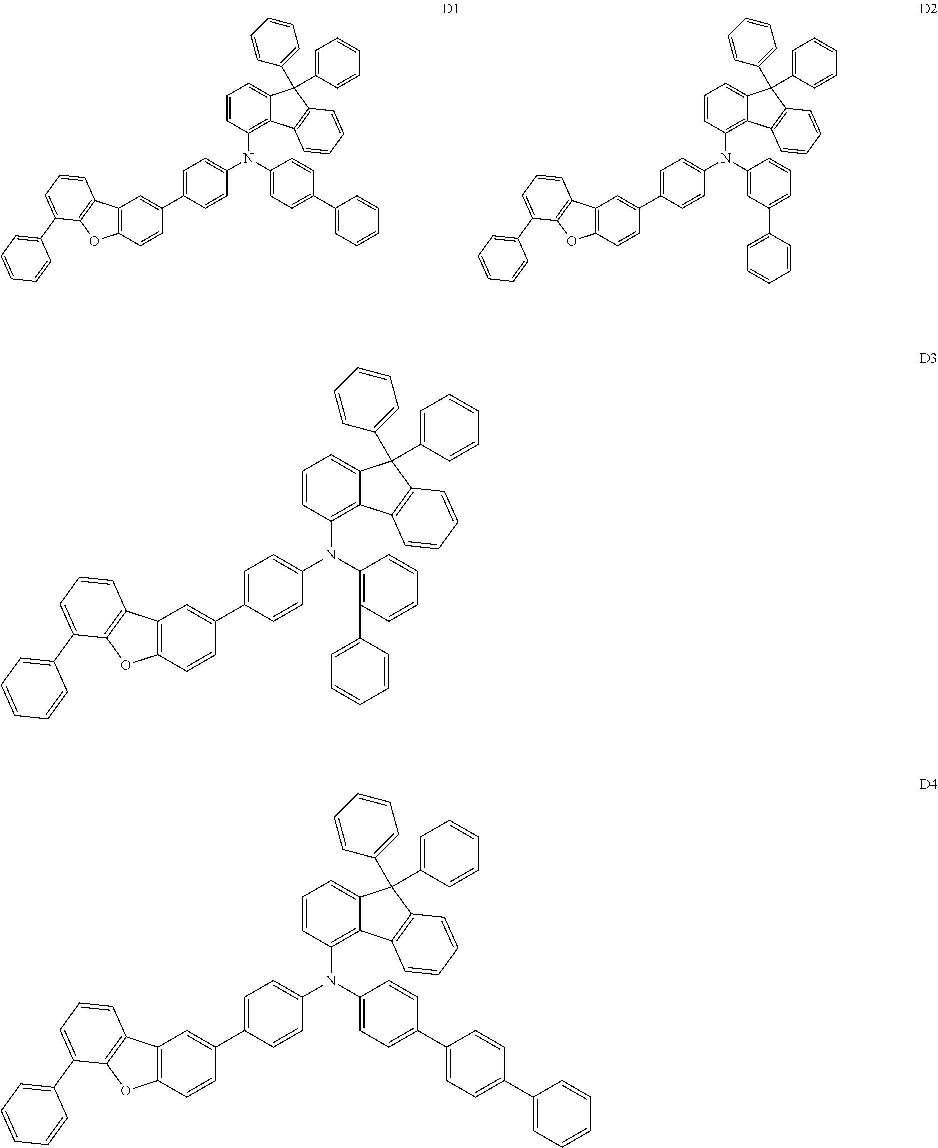 [SMILES: c1ccc(-c2ccc(N(c3ccc(-c4ccc5oc6c(-c7ccccc7)cccc6c5c4)cc3)c3cccc4c3-c3ccccc3C4(c3ccccc3)c3ccccc3)cc2)cc1 ; c1ccc(-c2cccc(N(c3ccc(-c4ccc5oc6c(-c7ccccc7)cccc6c5c4)cc3)c3cccc4c3-c3ccccc3C4(c3ccccc3)c3ccccc3)c2)cc1 ; c1ccc(-c2ccccc2N(c2ccc(-c3ccc4oc5c(-c6ccccc6)cccc5c4c3)cc2)c2cccc3c2-c2ccccc2C3(c2ccccc2)c2ccccc2)cc1 ; c1ccc(-c2ccc(-c3ccc(N(c4ccc(-c5ccc6oc7c(-c8ccccc8)cccc7c6c5)cc4)c4cccc5c4-c4ccccc4C5(c4ccccc4)c4ccccc4)cc3)cc2)cc1] 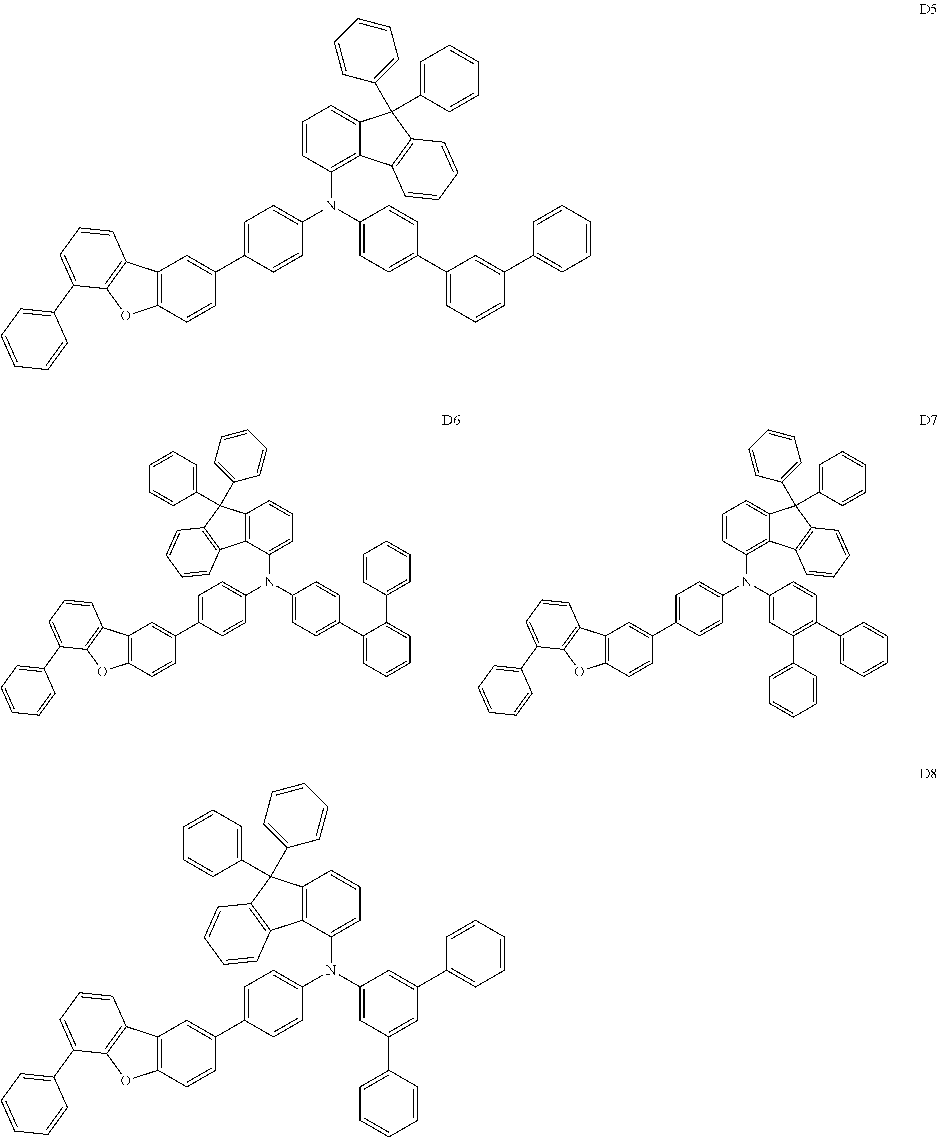 [SMILES: c1ccc(-c2cccc(-c3ccc(N(c4ccc(-c5ccc6oc7c(-c8ccccc8)cccc7c6c5)cc4)c4cccc5c4-c4ccccc4C5(c4ccccc4)c4ccccc4)cc3)c2)cc1 ; c1ccc(-c2ccccc2-c2ccc(N(c3ccc(-c4ccc5oc6c(-c7ccccc7)cccc6c5c4)cc3)c3cccc4c3-c3ccccc3C4(c3ccccc3)c3ccccc3)cc2)cc1 ; c1ccc(-c2ccc(N(c3ccc(-c4ccc5oc6c(-c7ccccc7)cccc6c5c4)cc3)c3cccc4c3-c3ccccc3C4(c3ccccc3)c3ccccc3)cc2-c2ccccc2)cc1 ; c1ccc(-c2cc(-c3ccccc3)cc(N(c3ccc(-c4ccc5oc6c(-c7ccccc7)cccc6c5c4)cc3)c3cccc4c3-c3ccccc3C4(c3ccccc3)c3ccccc3)c2)cc1] 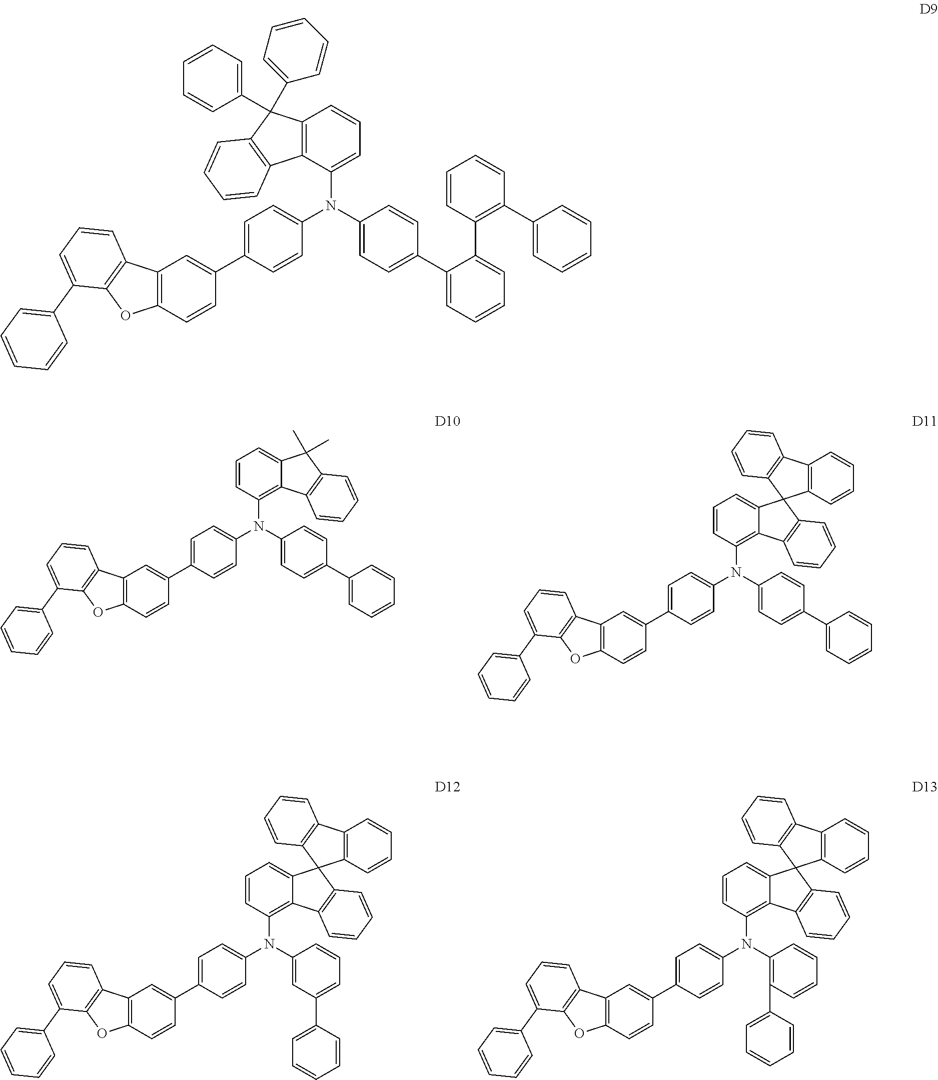 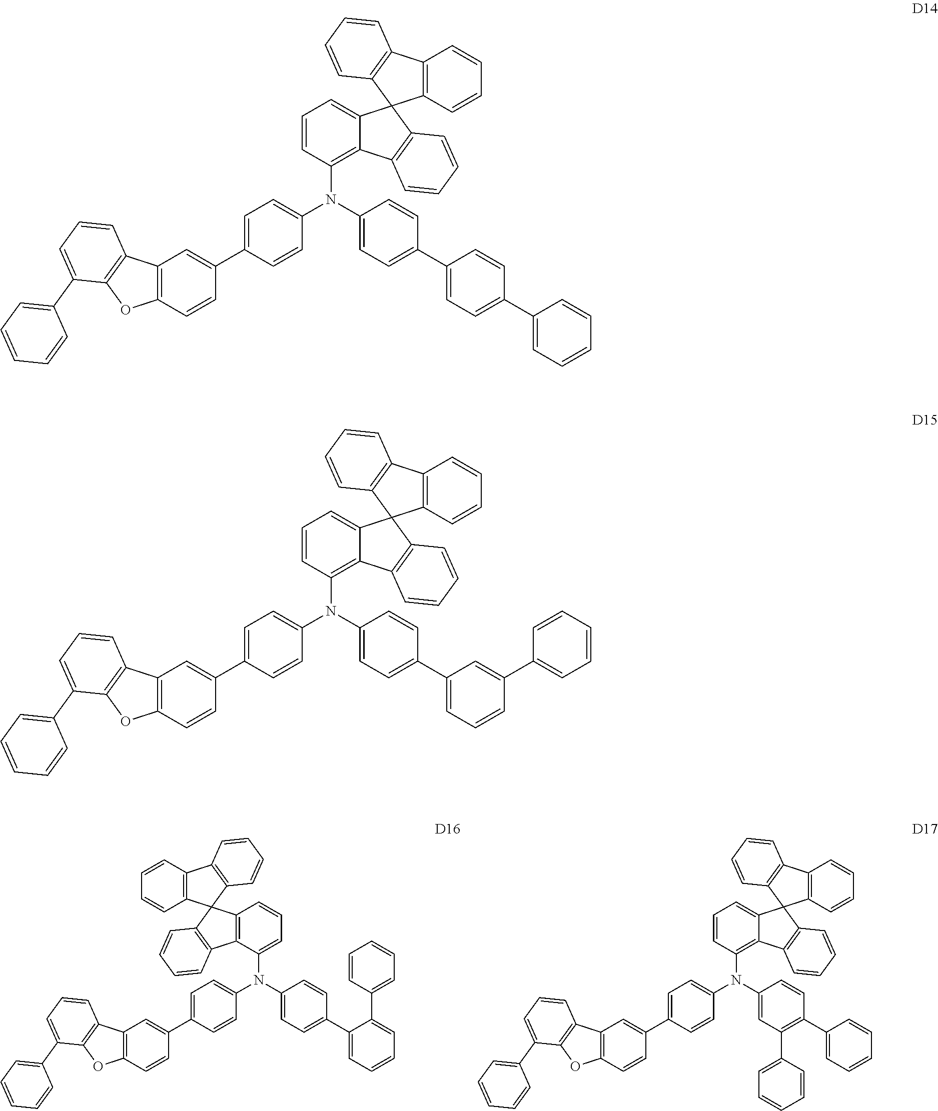 [SMILES: c1ccc(-c2ccc(-c3ccc(N(c4ccc(-c5ccc6oc7c(-c8ccccc8)cccc7c6c5)cc4)c4cccc5c4-c4ccccc4C54c5ccccc5-c5ccccc54)cc3)cc2)cc1 ; c1ccc(-c2cccc(-c3ccc(N(c4ccc(-c5ccc6oc7c(-c8ccccc8)cccc7c6c5)cc4)c4cccc5c4-c4ccccc4C54c5ccccc5-c5ccccc54)cc3)c2)cc1 ; c1ccc(-c2ccccc2-c2ccc(N(c3ccc(-c4ccc5oc6c(-c7ccccc7)cccc6c5c4)cc3)c3cccc4c3-c3ccccc3C43c4ccccc4-c4ccccc43)cc2)cc1 ; c1ccc(-c2ccc(N(c3ccc(-c4ccc5oc6c(-c7ccccc7)cccc6c5c4)cc3)c3cccc4c3-c3ccccc3C43c4ccccc4-c4ccccc43)cc2-c2ccccc2)cc1] 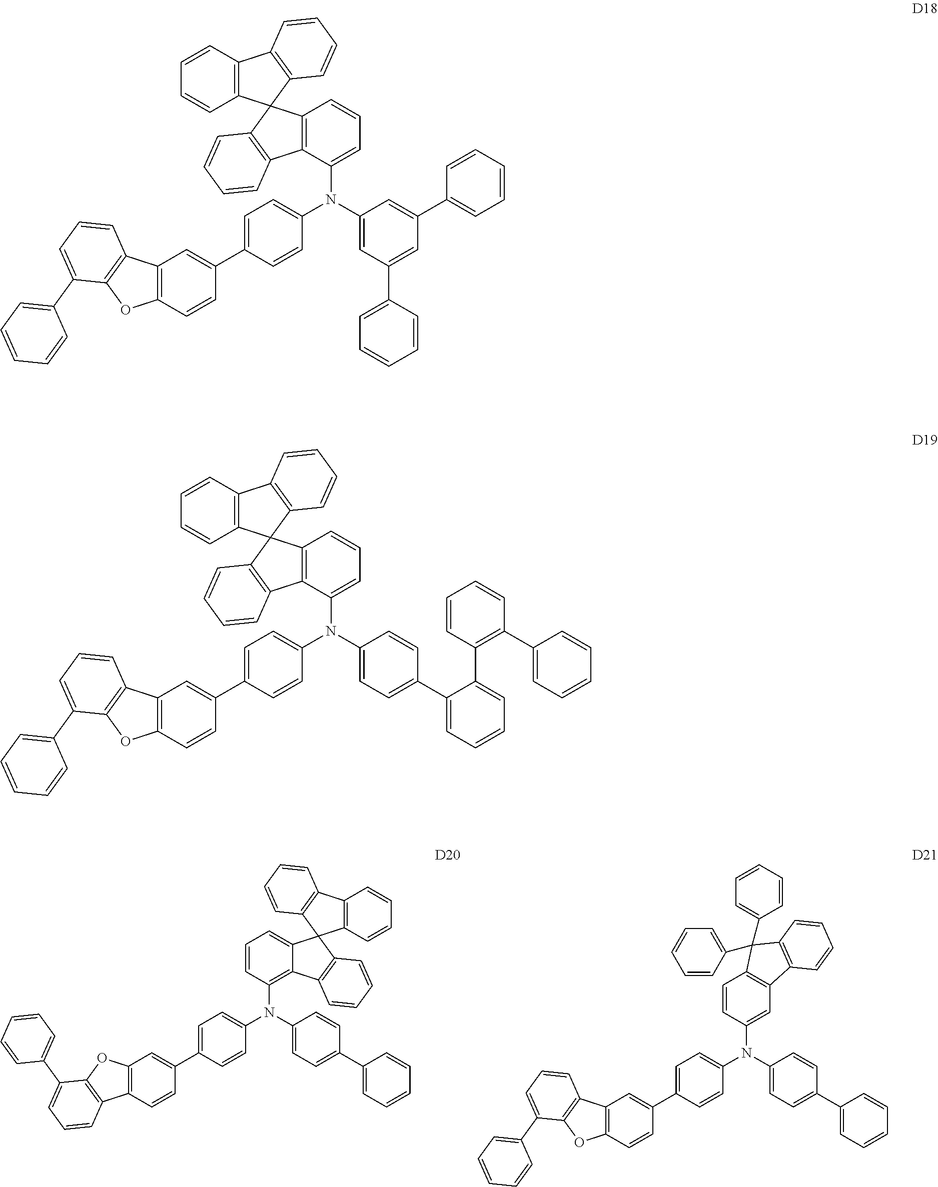 [SMILES: c1ccc(-c2cc(-c3ccccc3)cc(N(c3ccc(-c4ccc5oc6c(-c7ccccc7)cccc6c5c4)cc3)c3cccc4c3-c3ccccc3C43c4ccccc4-c4ccccc43)c2)cc1 ; c1ccc(-c2ccccc2-c2ccccc2-c2ccc(N(c3ccc(-c4ccc5oc6c(-c7ccccc7)cccc6c5c4)cc3)c3cccc4c3-c3ccccc3C43c4ccccc4-c4ccccc43)cc2)cc1 ; c1ccc(-c2ccc(N(c3ccc(-c4ccc5c(c4)oc4c(-c6ccccc6)cccc45)cc3)c3cccc4c3-c3ccccc3C43c4ccccc4-c4ccccc43)cc2)cc1 ; c1ccc(-c2ccc(N(c3ccc(-c4ccc5oc6c(-c7ccccc7)cccc6c5c4)cc3)c3ccc4c(c3)-c3ccccc3C4(c3ccccc3)c3ccccc3)cc2)cc1] 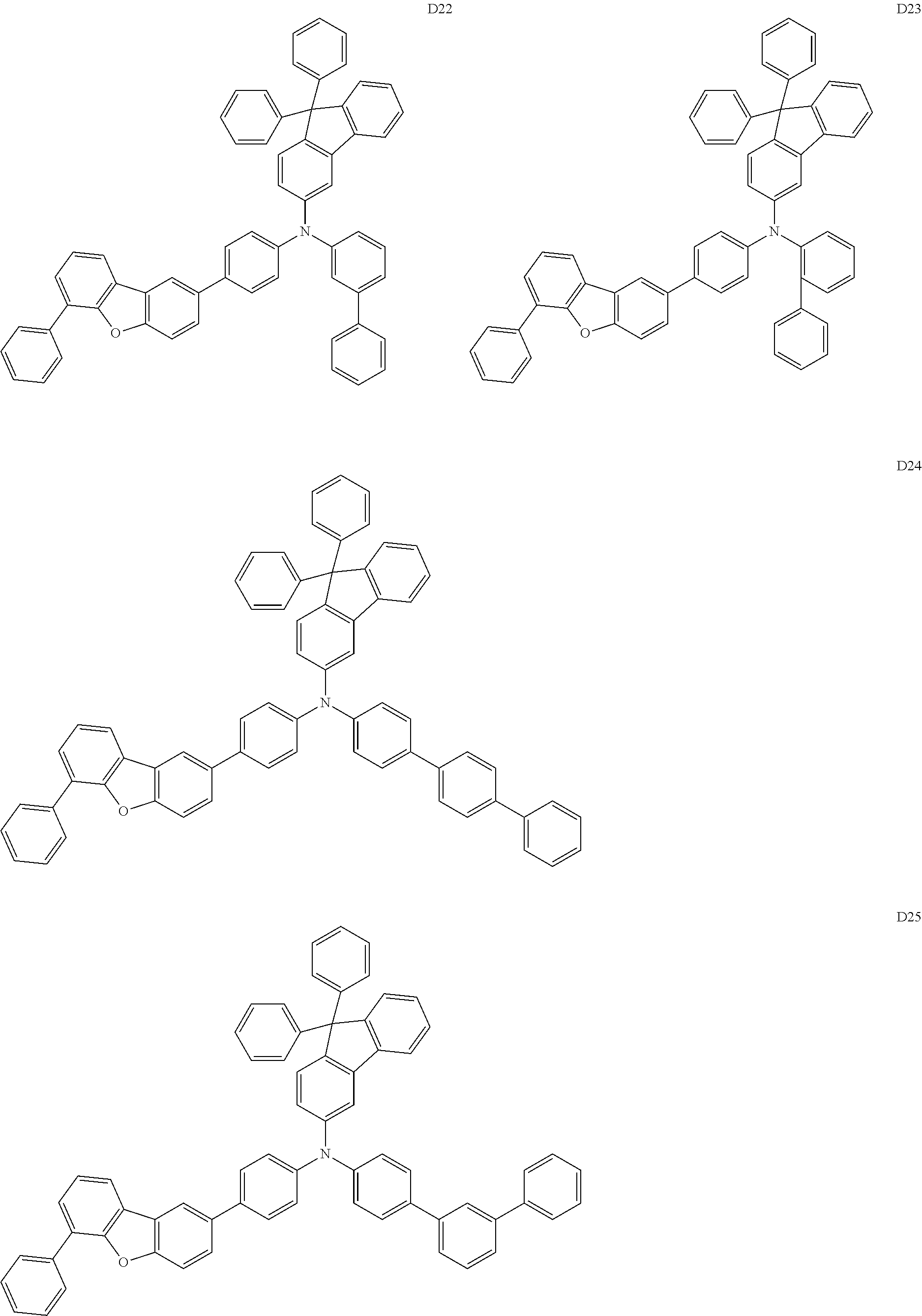 [SMILES: c1ccc(-c2cccc(N(c3ccc(-c4ccc5oc6c(-c7ccccc7)cccc6c5c4)cc3)c3ccc4c(c3)-c3ccccc3C4(c3ccccc3)c3ccccc3)c2)cc1 ; c1ccc(-c2ccccc2N(c2ccc(-c3ccc4oc5c(-c6ccccc6)cccc5c4c3)cc2)c2ccc3c(c2)-c2ccccc2C3(c2ccccc2)c2ccccc2)cc1 ; c1ccc(-c2ccc(-c3ccc(N(c4ccc(-c5ccc6oc7c(-c8ccccc8)cccc7c6c5)cc4)c4ccc5c(c4)-c4ccccc4C5(c4ccccc4)c4ccccc4)cc3)cc2)cc1 ; c1ccc(-c2cccc(-c3ccc(N(c4ccc(-c5ccc6oc7c(-c8ccccc8)cccc7c6c5)cc4)c4ccc5c(c4)-c4ccccc4C5(c4ccccc4)c4ccccc4)cc3)c2)cc1] 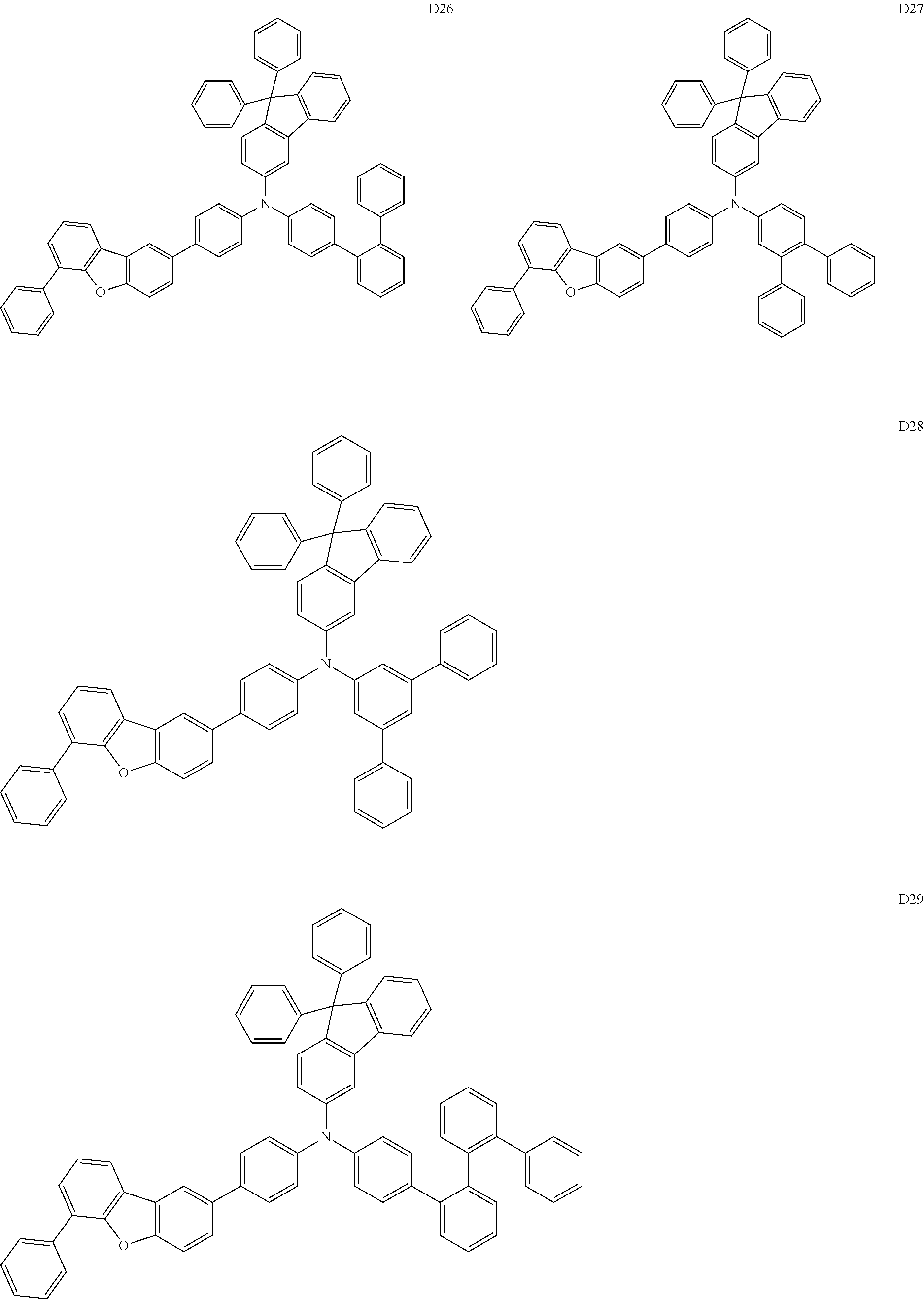 [SMILES: c1ccc(-c2ccccc2-c2ccc(N(c3ccc(-c4ccc5oc6c(-c7ccccc7)cccc6c5c4)cc3)c3ccc4c(c3)-c3ccccc3C4(c3ccccc3)c3ccccc3)cc2)cc1 ; c1ccc(-c2ccc(N(c3ccc(-c4ccc5oc6c(-c7ccccc7)cccc6c5c4)cc3)c3ccc4c(c3)-c3ccccc3C4(c3ccccc3)c3ccccc3)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc4c(c3)-c3ccccc3C4(c3ccccc3)c3ccccc3)c2)cc1 ; c1ccc(-c2ccccc2-c2ccccc2-c2ccc(N(c3ccc(-c4ccc5oc6c(-c7ccccc7)cccc6c5c4)cc3)c3ccc4c(c3)-c3ccccc3C4(c3ccccc3)c3ccccc3)cc2)cc1] 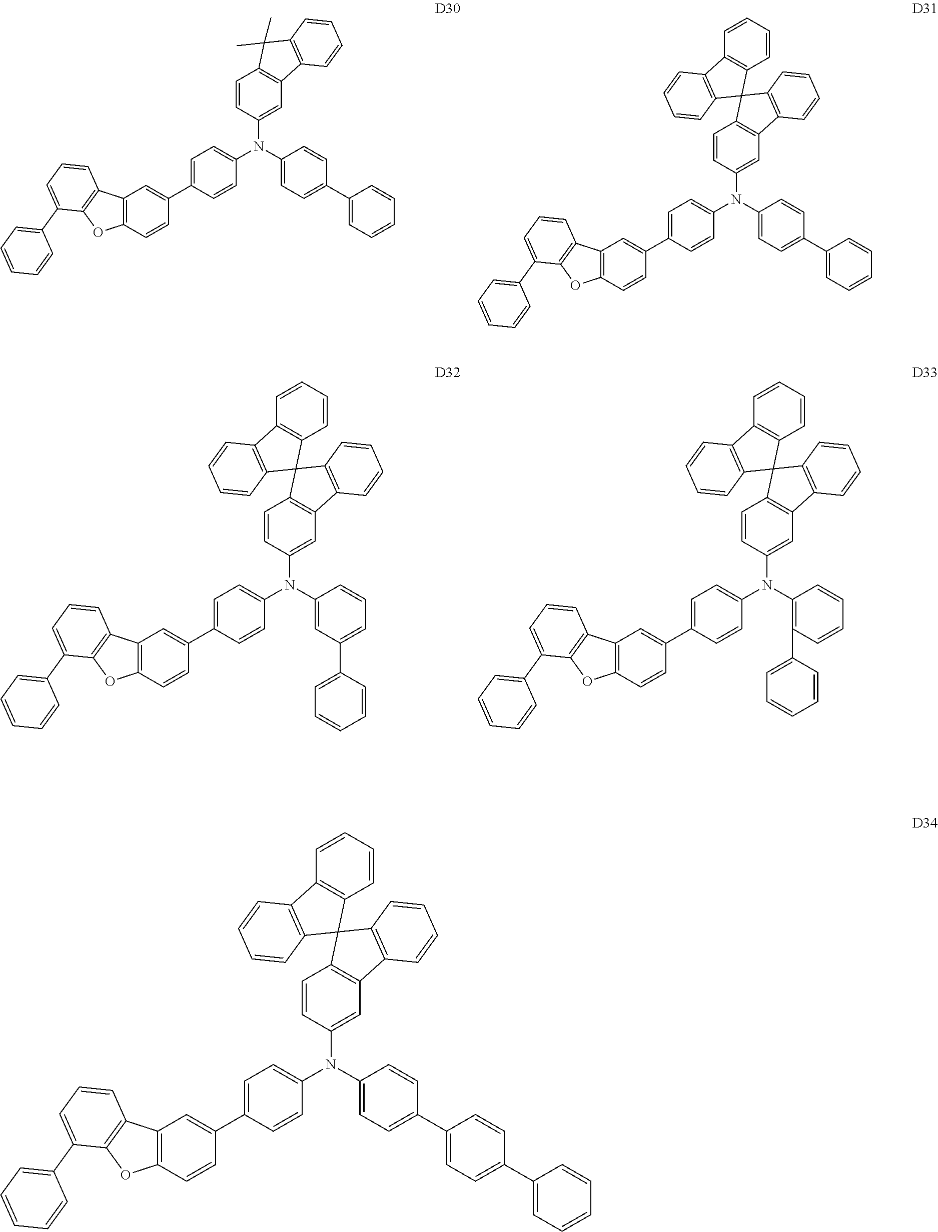 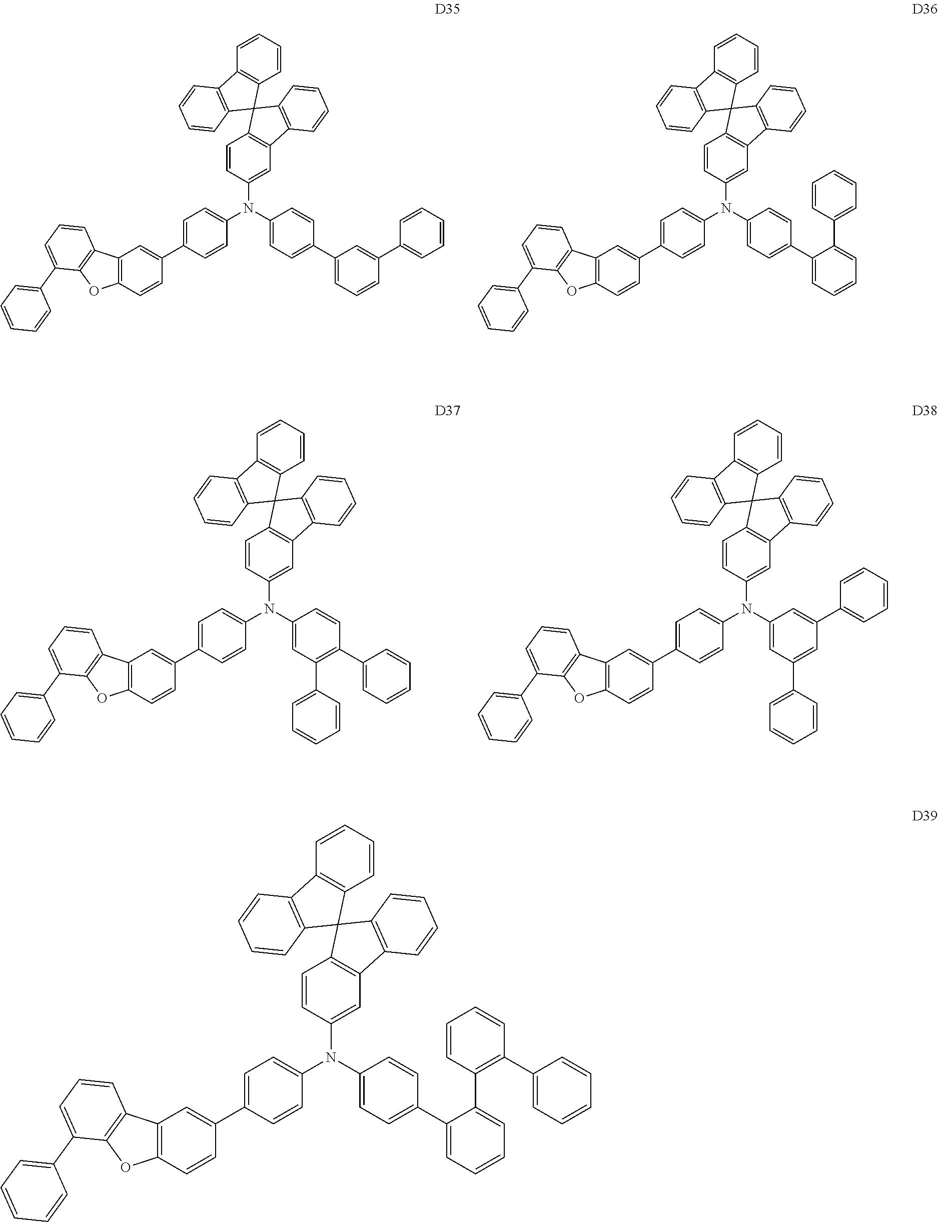 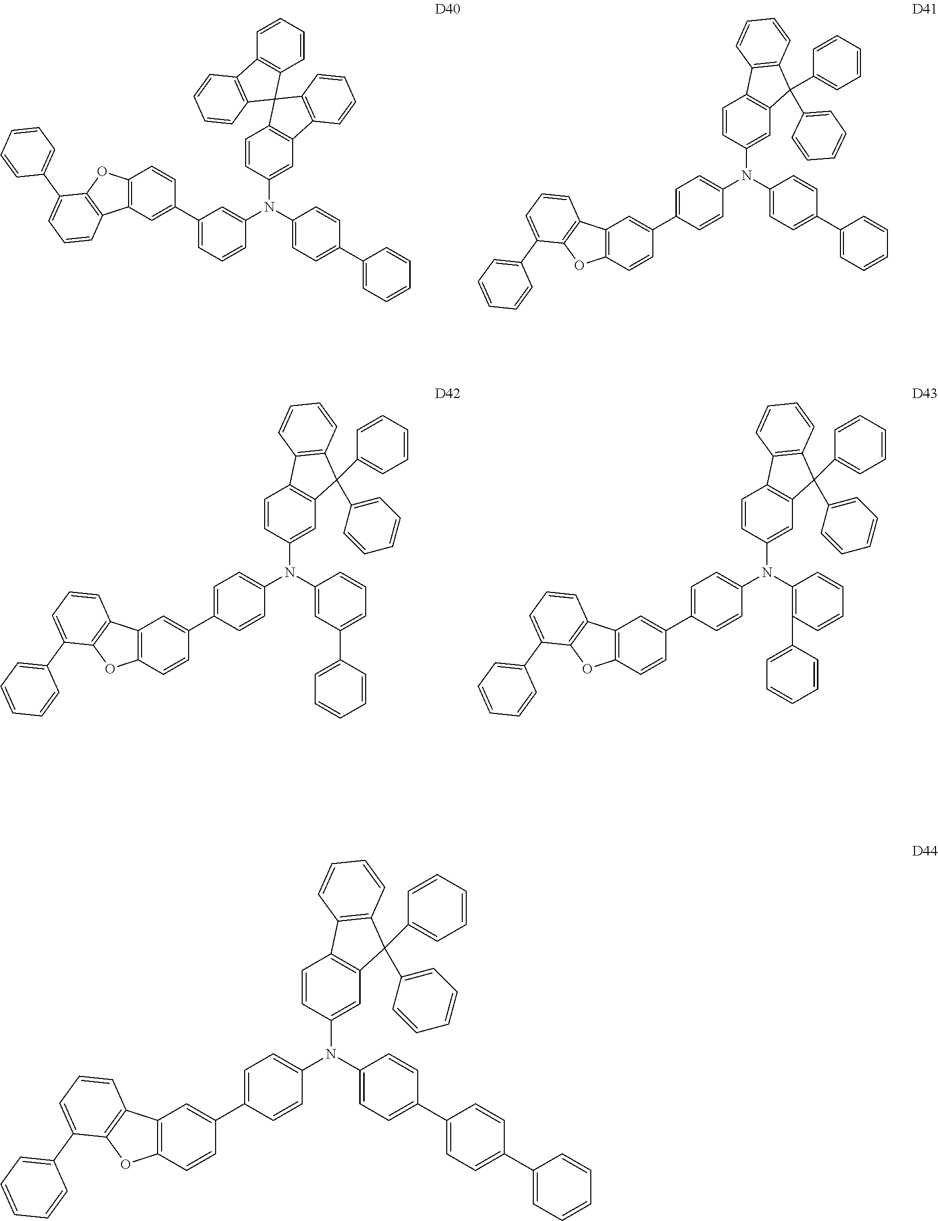 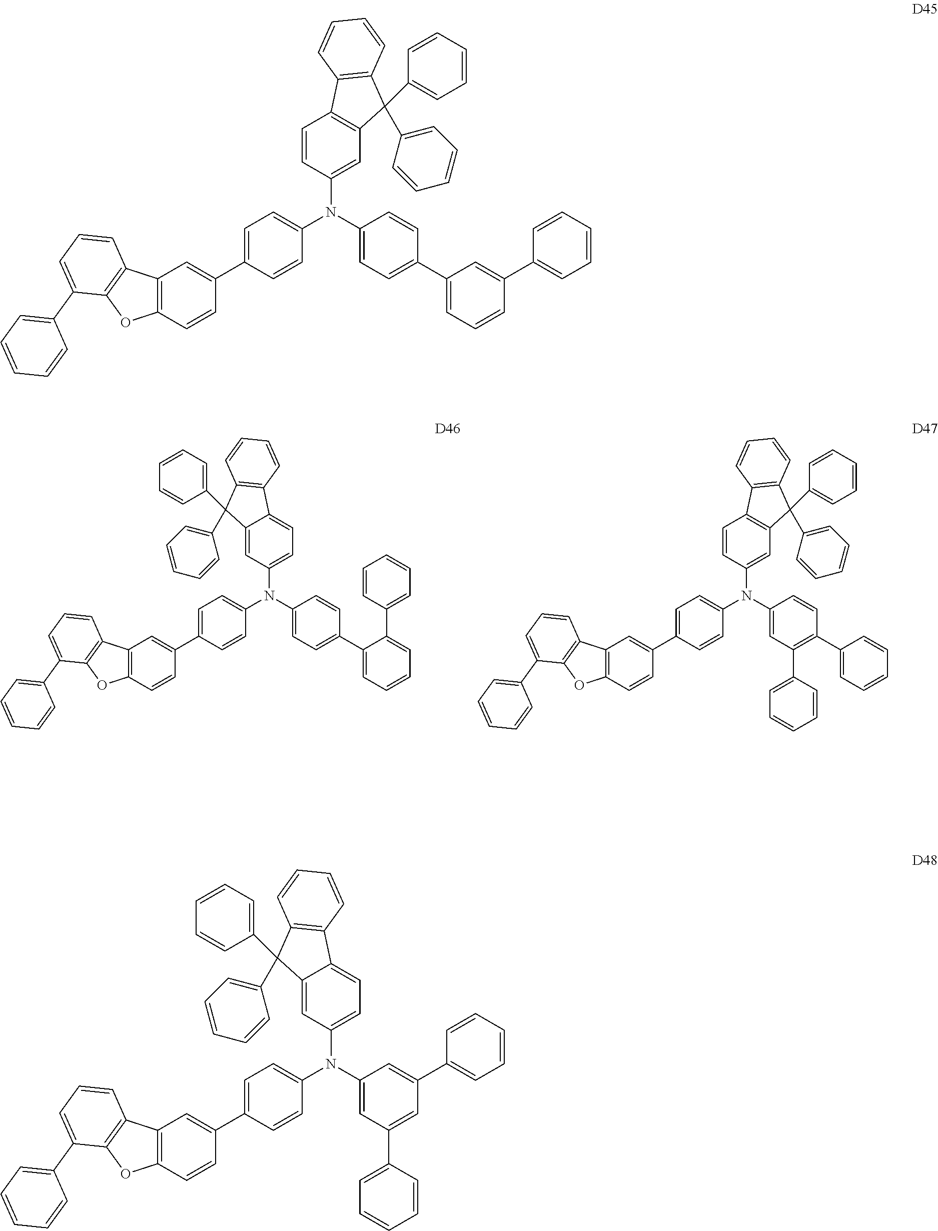 [SMILES: c1ccc(-c2cccc(-c3ccc(N(c4ccc(-c5ccc6oc7c(-c8ccccc8)cccc7c6c5)cc4)c4ccc5c(c4)C(c4ccccc4)(c4ccccc4)c4ccccc4-5)cc3)c2)cc1 ; c1ccc(-c2ccccc2-c2ccc(N(c3ccc(-c4ccc5oc6c(-c7ccccc7)cccc6c5c4)cc3)c3ccc4c(c3)C(c3ccccc3)(c3ccccc3)c3ccccc3-4)cc2)cc1 ; c1ccc(-c2ccc(N(c3ccc(-c4ccc5oc6c(-c7ccccc7)cccc6c5c4)cc3)c3ccc4c(c3)C(c3ccccc3)(c3ccccc3)c3ccccc3-4)cc2-c2ccccc2)cc1 ; c1ccc(-c2cc(-c3ccccc3)cc(N(c3ccc(-c4ccc5oc6c(-c7ccccc7)cccc6c5c4)cc3)c3ccc4c(c3)C(c3ccccc3)(c3ccccc3)c3ccccc3-4)c2)cc1] 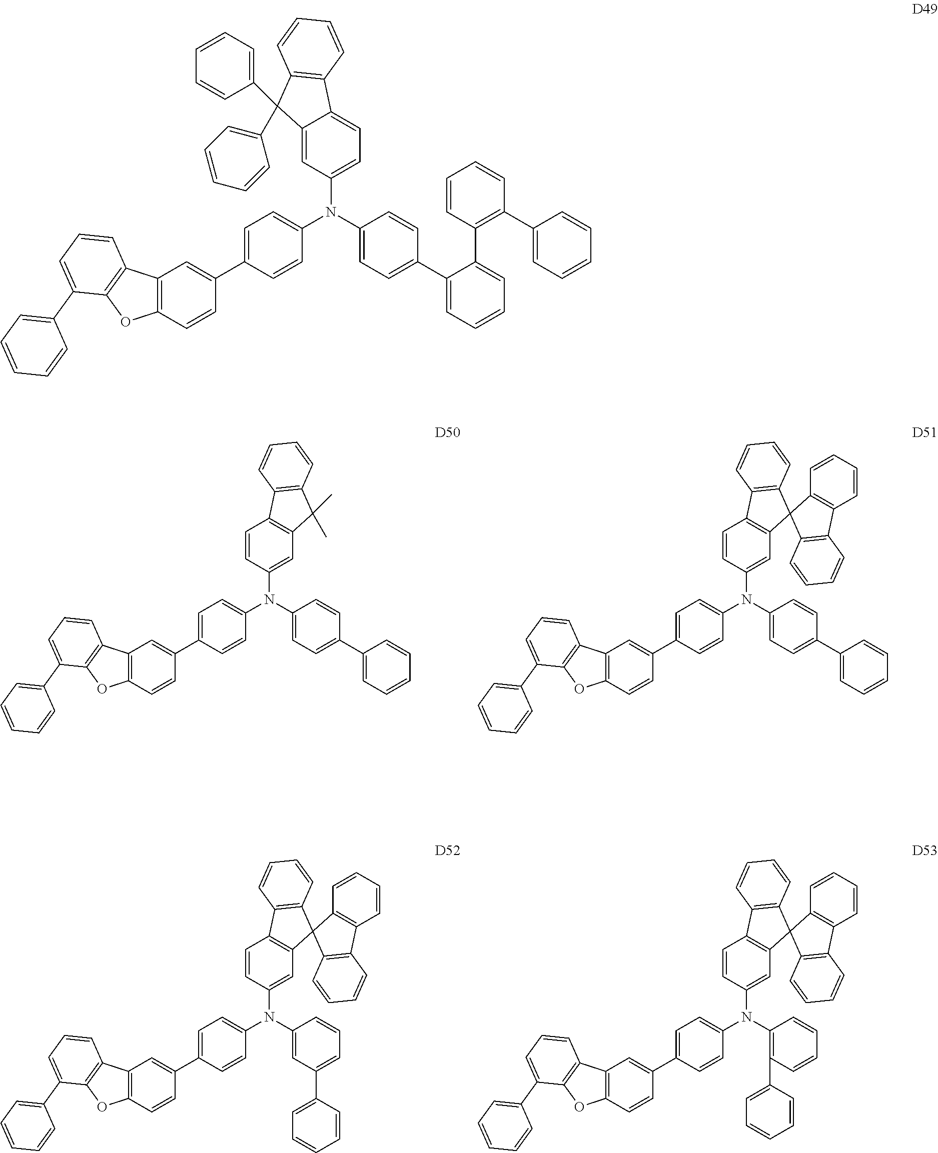 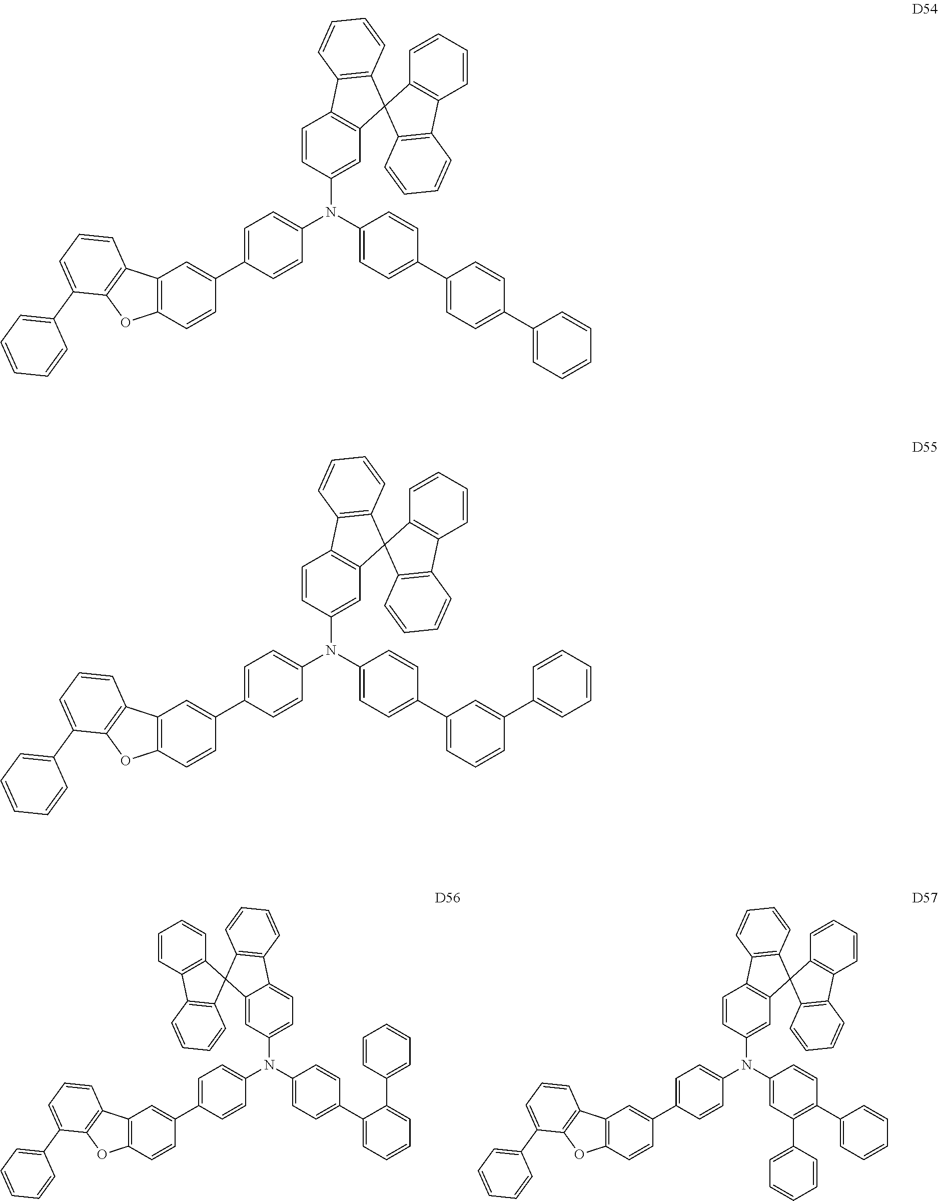 [SMILES: c1ccc(-c2ccc(-c3ccc(N(c4ccc(-c5ccc6oc7c(-c8ccccc8)cccc7c6c5)cc4)c4ccc5c(c4)C4(c6ccccc6-c6ccccc64)c4ccccc4-5)cc3)cc2)cc1 ; c1ccc(-c2cccc(-c3ccc(N(c4ccc(-c5ccc6oc7c(-c8ccccc8)cccc7c6c5)cc4)c4ccc5c(c4)C4(c6ccccc6-c6ccccc64)c4ccccc4-5)cc3)c2)cc1 ; c1ccc(-c2ccccc2-c2ccc(N(c3ccc(-c4ccc5oc6c(-c7ccccc7)cccc6c5c4)cc3)c3ccc4c(c3)C3(c5ccccc5-c5ccccc53)c3ccccc3-4)cc2)cc1 ; c1ccc(-c2ccc(N(c3ccc(-c4ccc5oc6c(-c7ccccc7)cccc6c5c4)cc3)c3ccc4c(c3)C3(c5ccccc5-c5ccccc53)c3ccccc3-4)cc2-c2ccccc2)cc1] 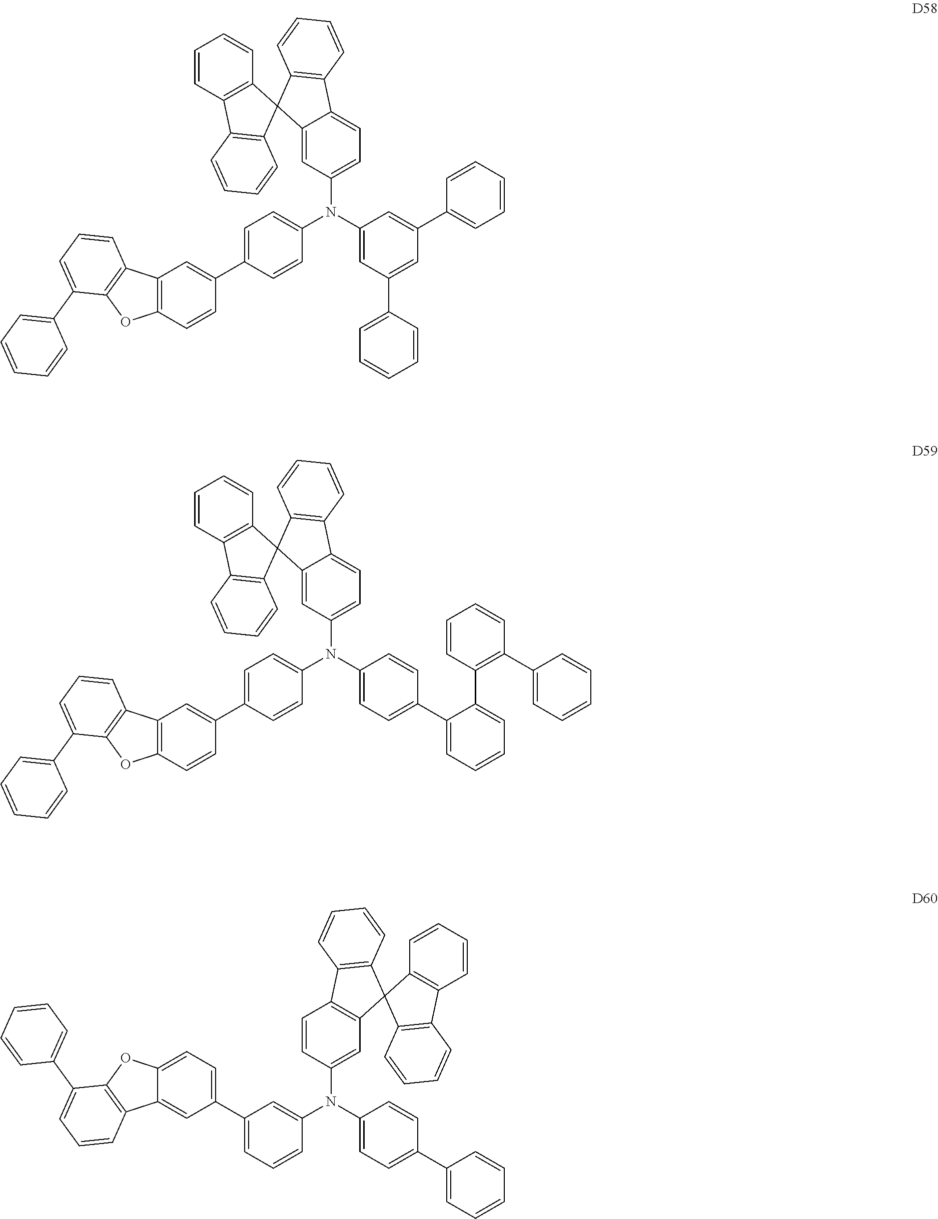 [SMILES: c1ccc(-c2cc(-c3ccccc3)cc(N(c3ccc(-c4ccc5oc6c(-c7ccccc7)cccc6c5c4)cc3)c3ccc4c(c3)C3(c5ccccc5-c5ccccc53)c3ccccc3-4)c2)cc1 ; c1ccc(-c2ccccc2-c2ccccc2-c2ccc(N(c3ccc(-c4ccc5oc6c(-c7ccccc7)cccc6c5c4)cc3)c3ccc4c(c3)C3(c5ccccc5-c5ccccc53)c3ccccc3-4)cc2)cc1 ; c1ccc(-c2ccc(N(c3cccc(-c4ccc5oc6c(-c7ccccc7)cccc6c5c4)c3)c3ccc4c(c3)C3(c5ccccc5-c5ccccc53)c3ccccc3-4)cc2)cc1] 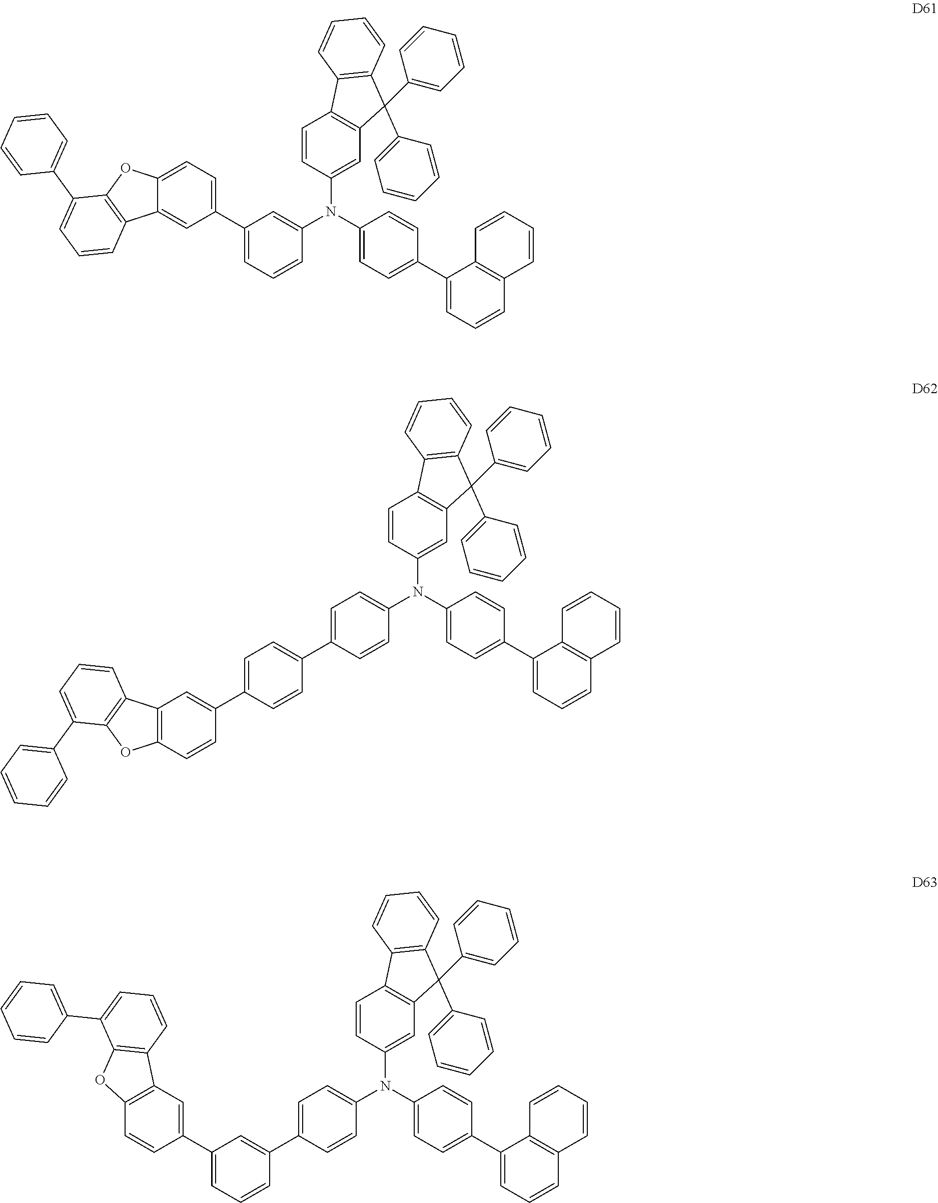 [SMILES: c1ccc(-c2cccc3c2oc2ccc(-c4cccc(N(c5ccc(-c6cccc7ccccc67)cc5)c5ccc6c(c5)C(c5ccccc5)(c5ccccc5)c5ccccc5-6)c4)cc23)cc1 ; c1ccc(-c2cccc3c2oc2ccc(-c4ccc(-c5ccc(N(c6ccc(-c7cccc8ccccc78)cc6)c6ccc7c(c6)C(c6ccccc6)(c6ccccc6)c6ccccc6-7)cc5)cc4)cc23)cc1 ; c1ccc(-c2cccc3c2oc2ccc(-c4cccc(-c5ccc(N(c6ccc(-c7cccc8ccccc78)cc6)c6ccc7c(c6)C(c6ccccc6)(c6ccccc6)c6ccccc6-7)cc5)c4)cc23)cc1] 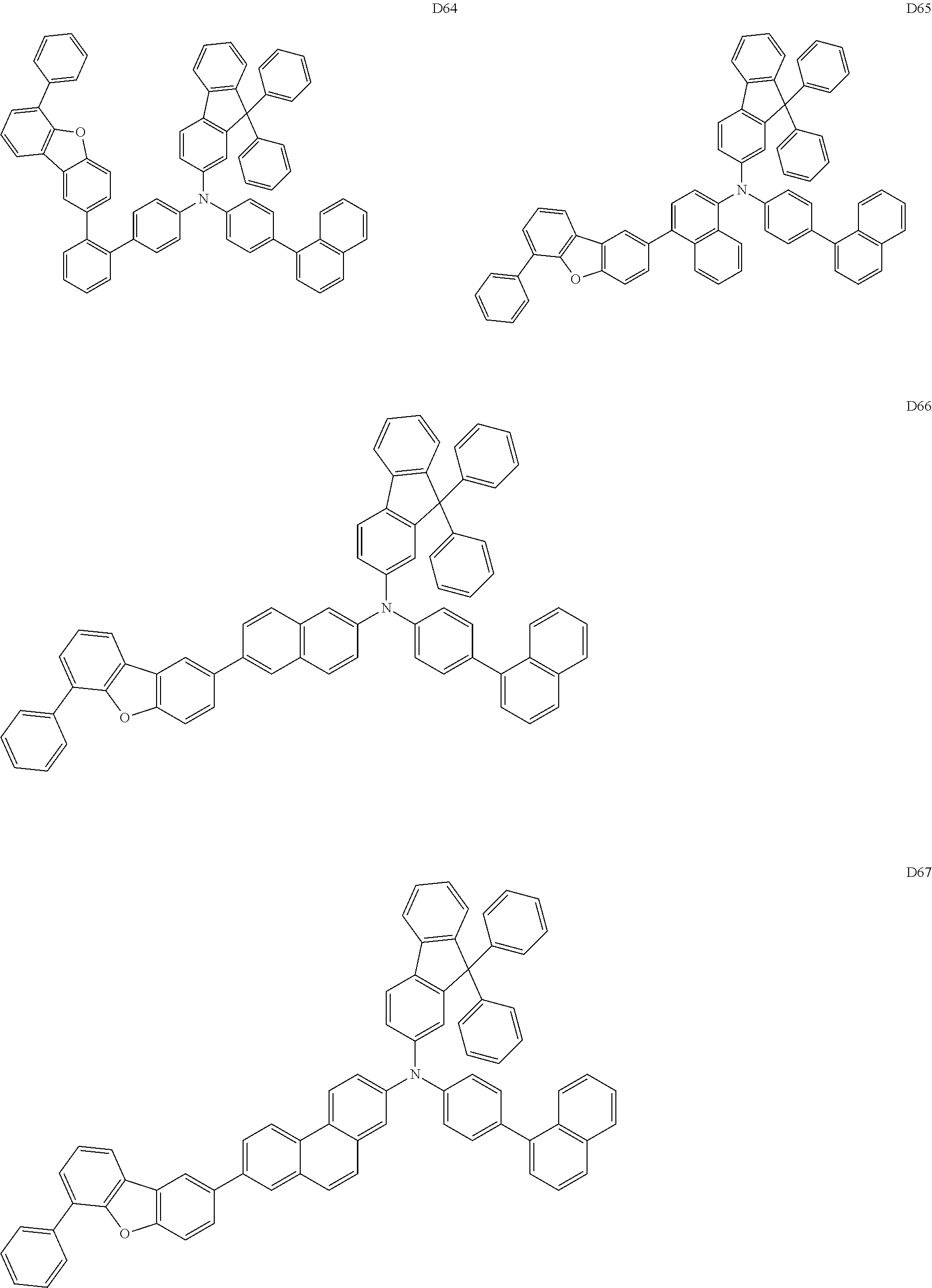 [SMILES: c1ccc(-c2cccc3c2oc2ccc(-c4ccccc4-c4ccc(N(c5ccc(-c6cccc7ccccc67)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6cccc7ccccc67)cc5)c5ccc6c(c5)C(c5ccccc5)(c5ccccc5)c5ccccc5-6)c5ccccc45)cc23)cc1 ; c1ccc(-c2cccc3c2oc2ccc(-c4ccc5cc(N(c6ccc(-c7cccc8ccccc78)cc6)c6ccc7c(c6)C(c6ccccc6)(c6ccccc6)c6ccccc6-7)ccc5c4)cc23)cc1 ; c1ccc(-c2cccc3c2oc2ccc(-c4ccc5c(ccc6cc(N(c7ccc(-c8cccc9ccccc89)cc7)c7ccc8c(c7)C(c7ccccc7)(c7ccccc7)c7ccccc7-8)ccc65)c4)cc23)cc1] 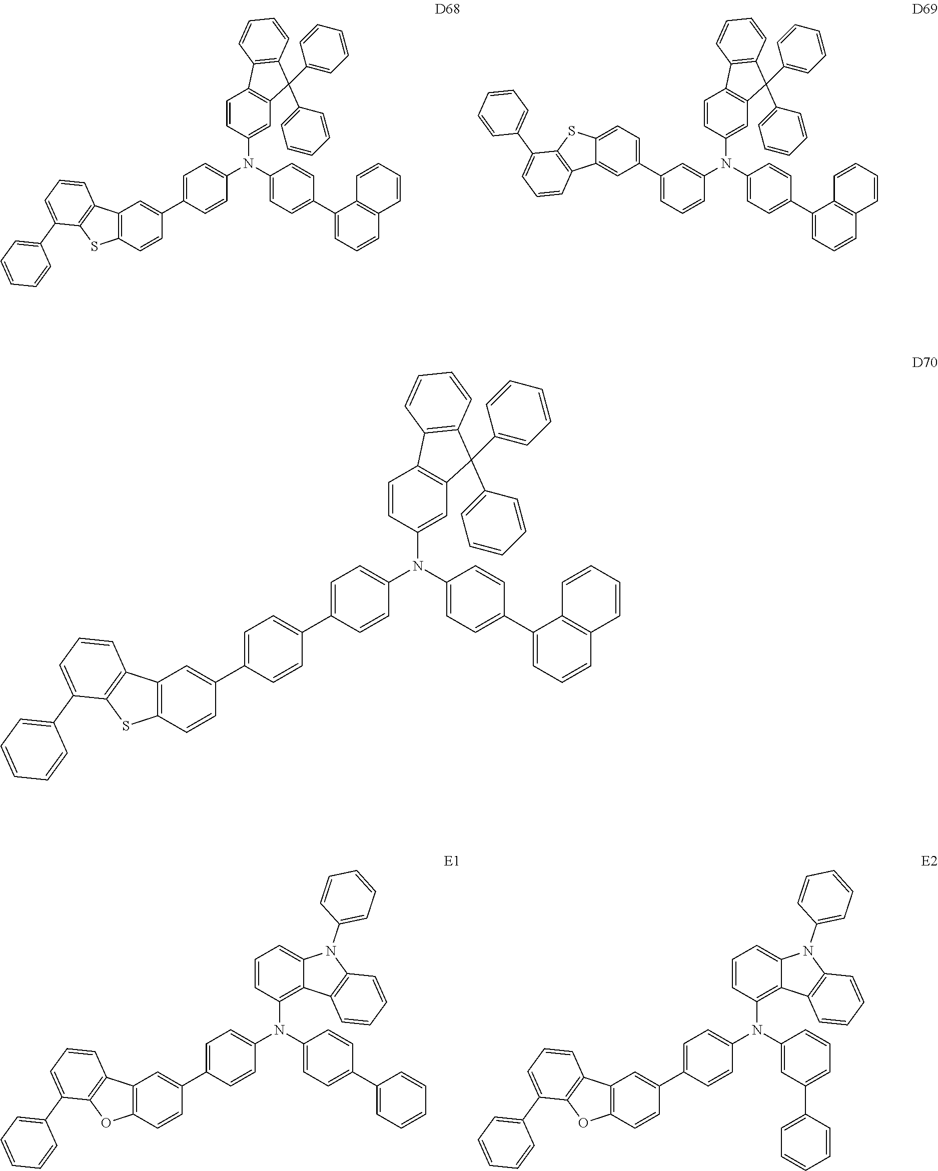 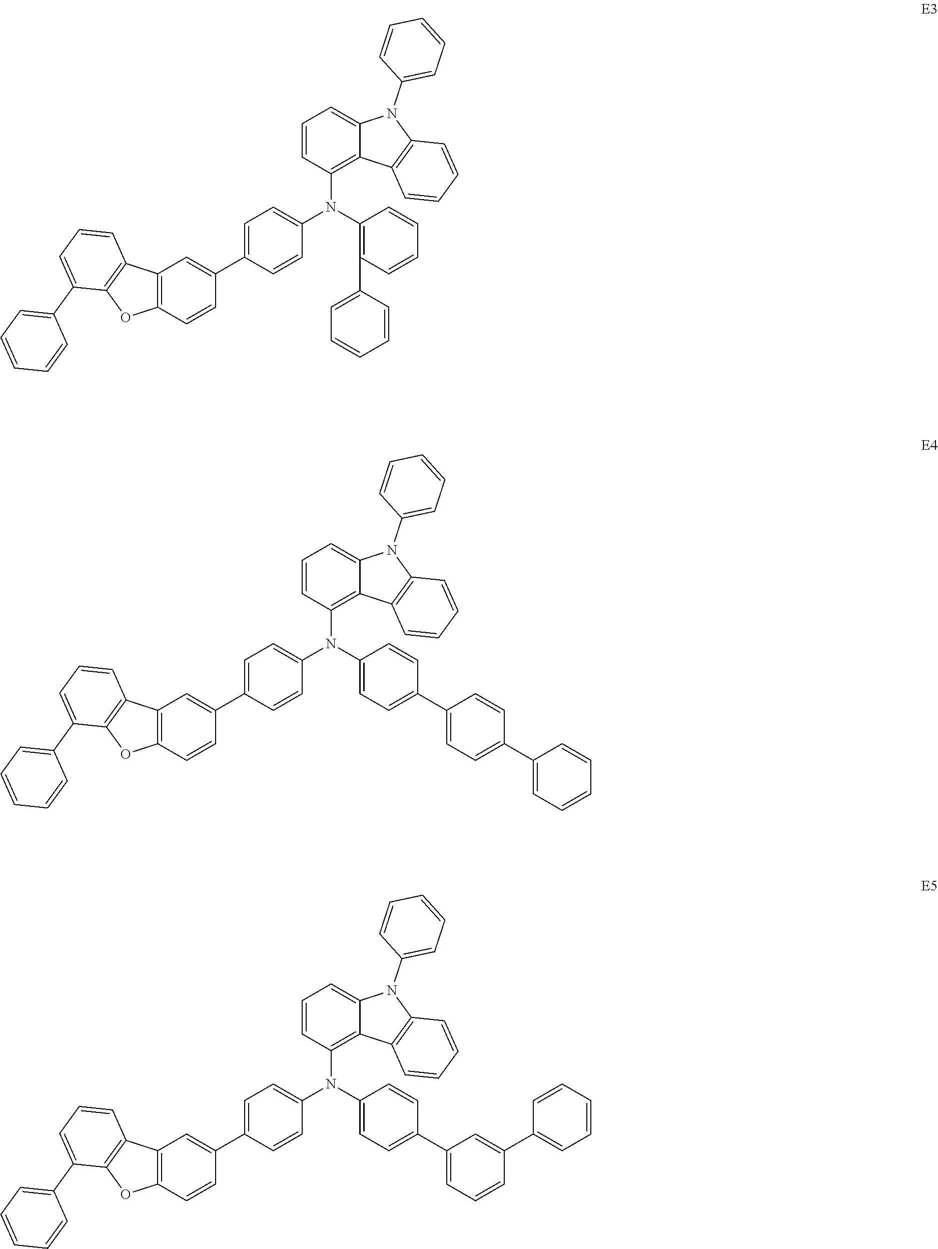 [SMILES: c1ccc(-c2ccccc2N(c2ccc(-c3ccc4oc5c(-c6ccccc6)cccc5c4c3)cc2)c2cccc3c2c2ccccc2n3-c2ccccc2)cc1 ; c1ccc(-c2ccc(-c3ccc(N(c4ccc(-c5ccc6oc7c(-c8ccccc8)cccc7c6c5)cc4)c4cccc5c4c4ccccc4n5-c4ccccc4)cc3)cc2)cc1 ; c1ccc(-c2cccc(-c3ccc(N(c4ccc(-c5ccc6oc7c(-c8ccccc8)cccc7c6c5)cc4)c4cccc5c4c4ccccc4n5-c4ccccc4)cc3)c2)cc1] 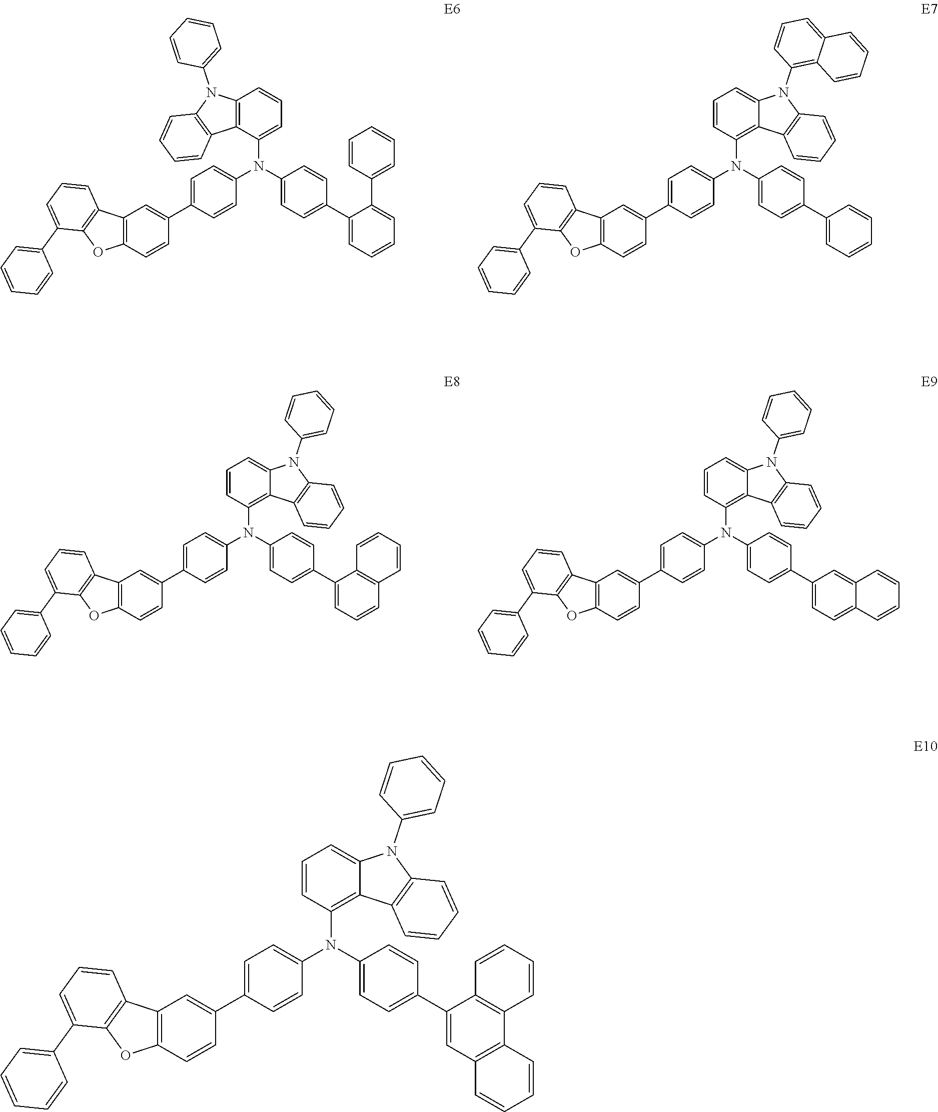 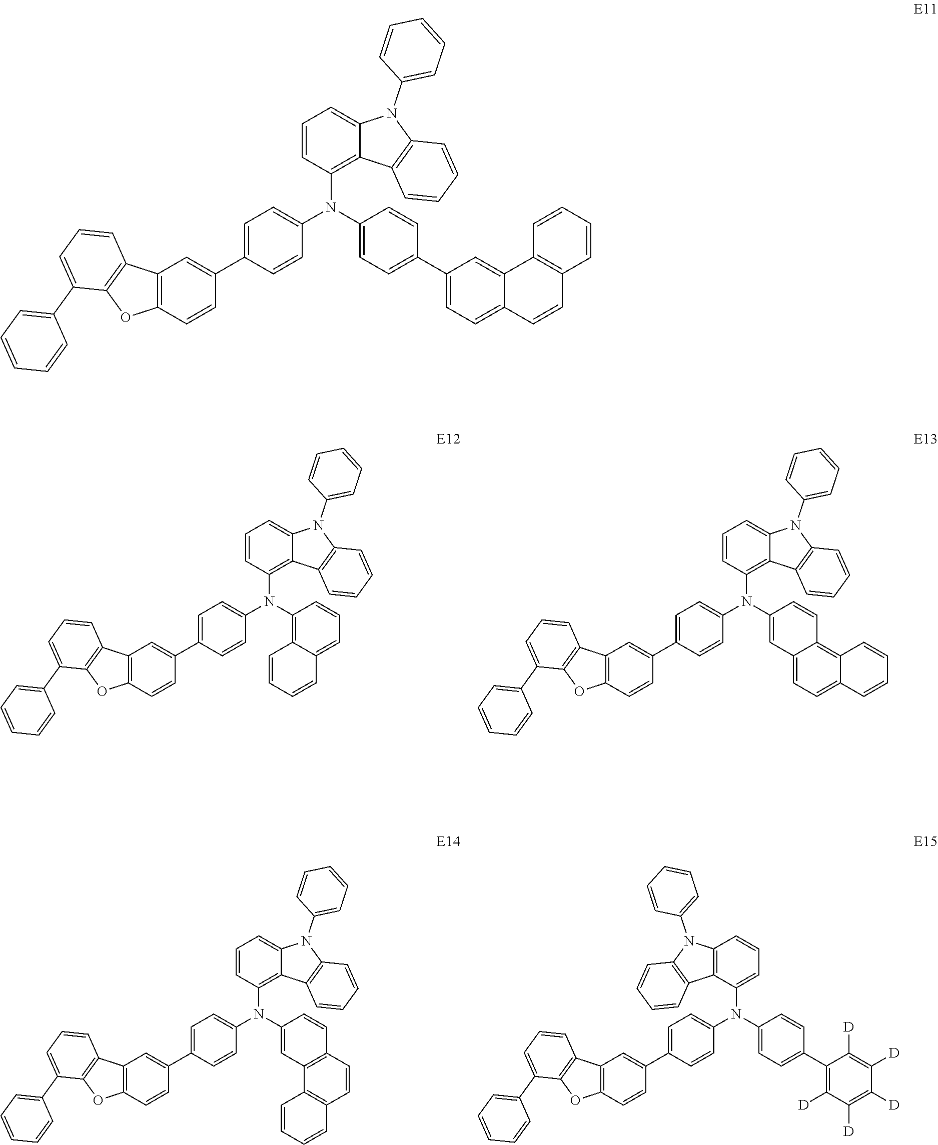 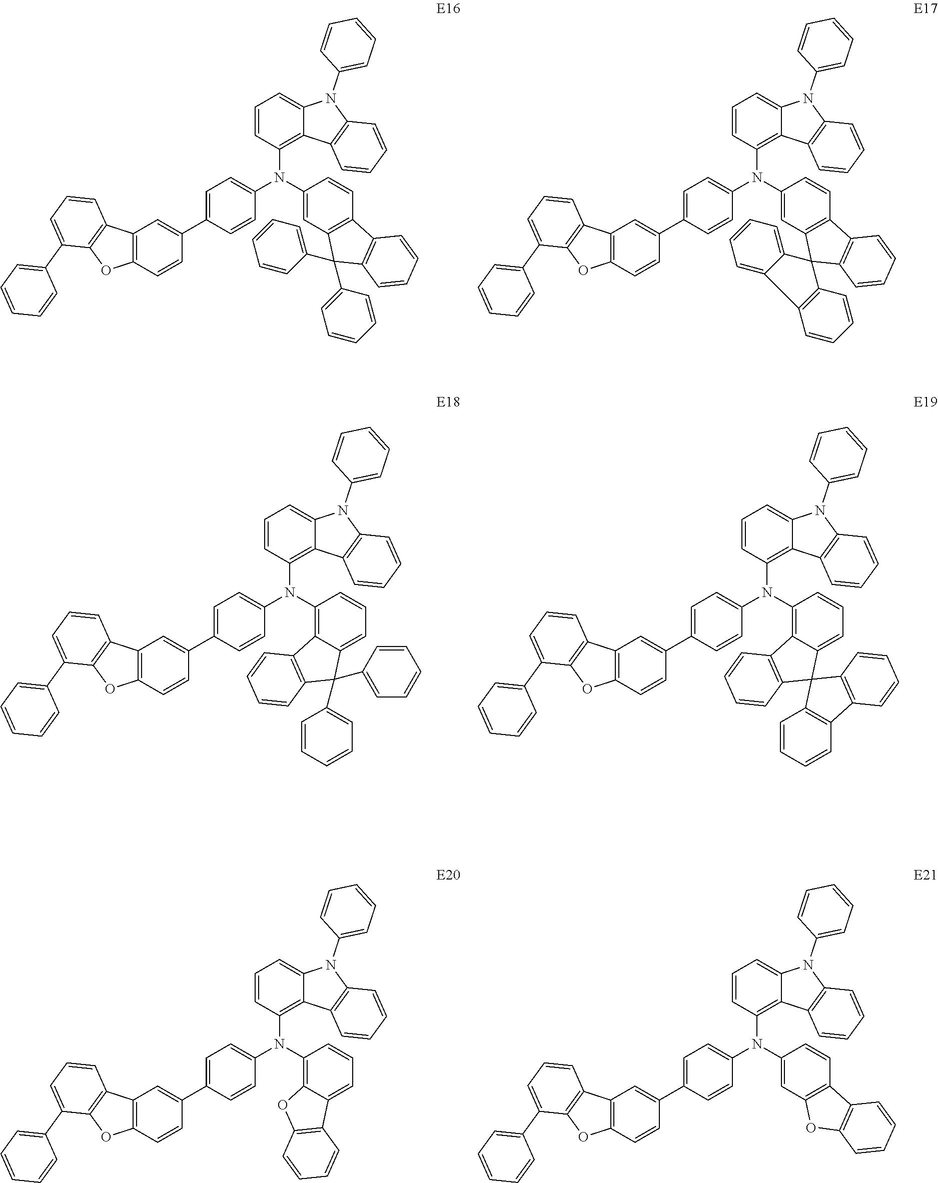 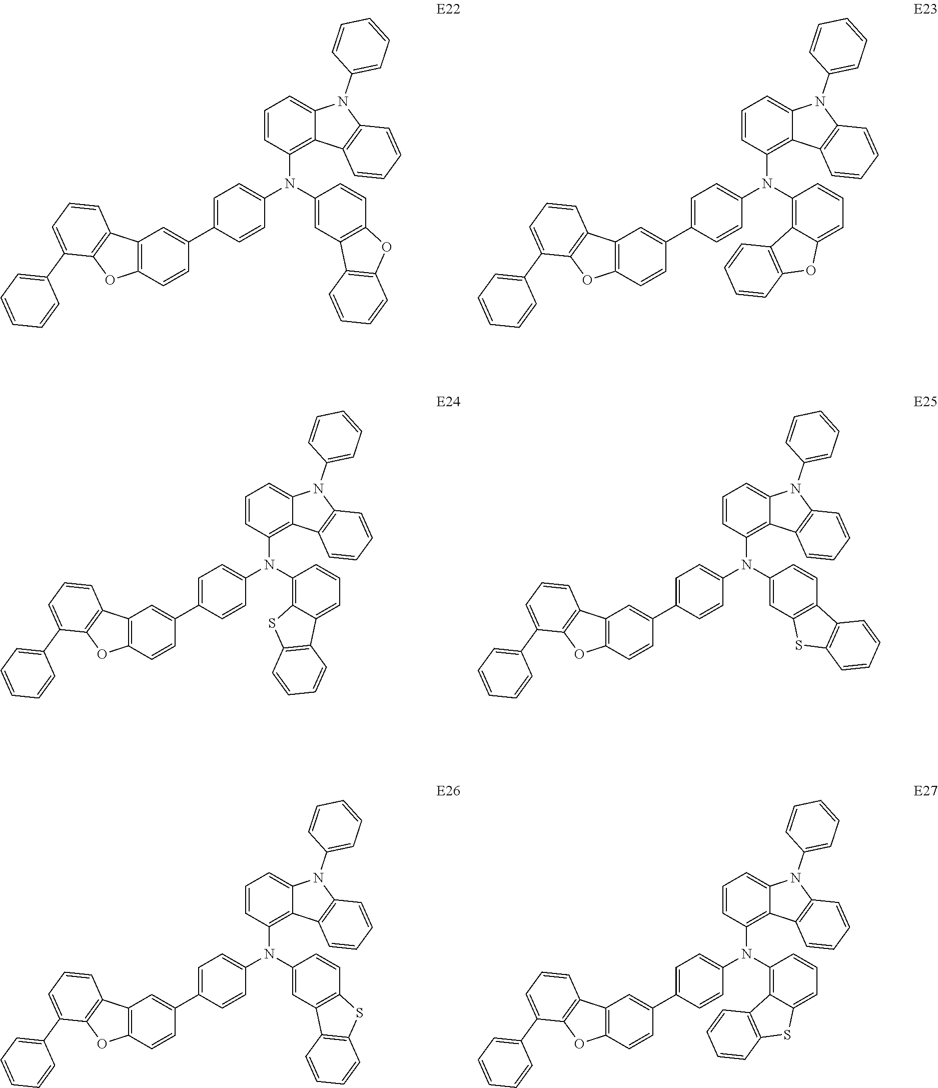 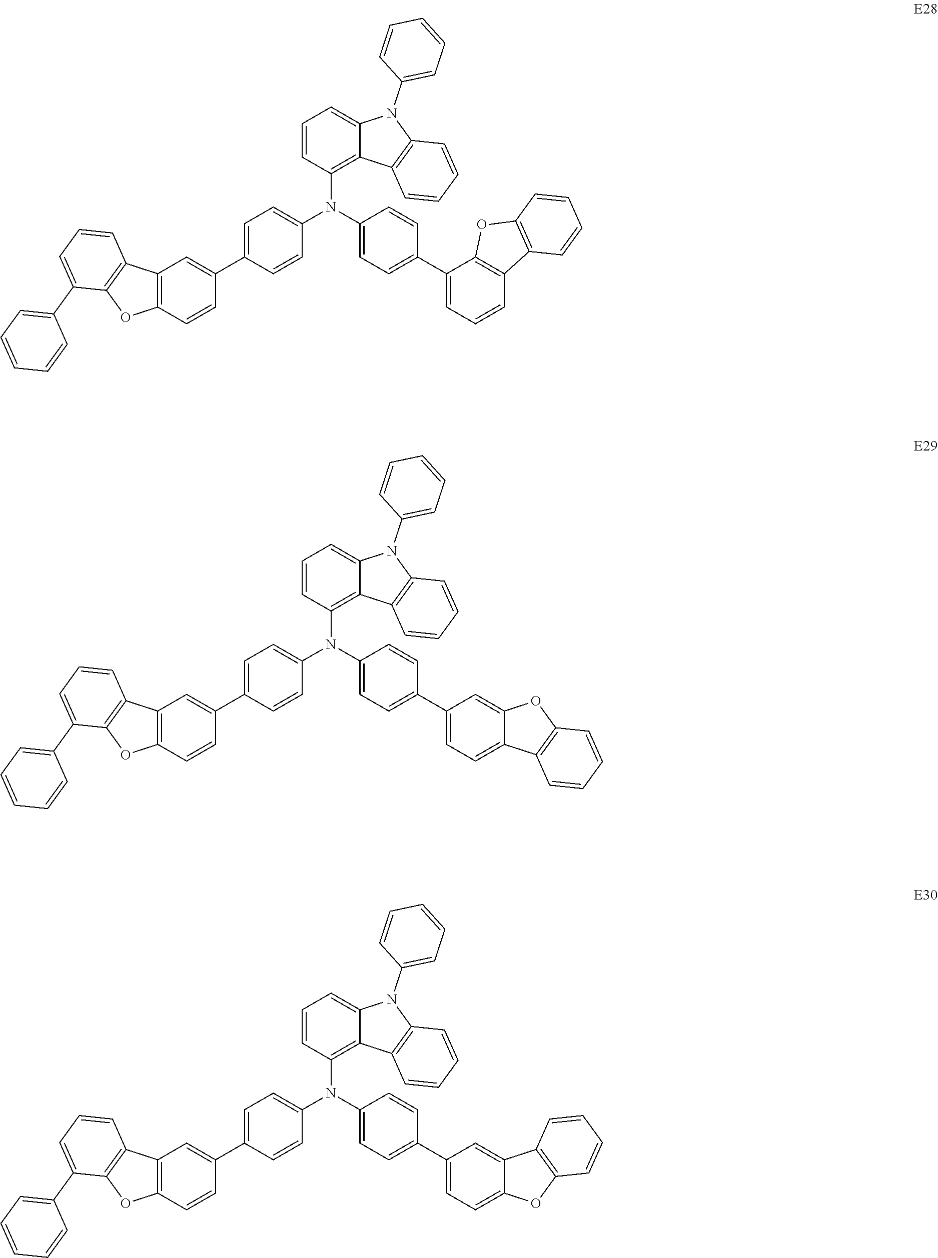 [SMILES: c1ccc(-c2cccc3c2oc2ccc(-c4ccc(N(c5ccc(-c6cccc7c6oc6ccccc67)cc5)c5cccc6c5c5ccccc5n6-c5ccccc5)cc4)cc23)cc1 ; c1ccc(-c2cccc3c2oc2ccc(-c4ccc(N(c5ccc(-c6ccc7c(c6)oc6ccccc67)cc5)c5cccc6c5c5ccccc5n6-c5ccccc5)cc4)cc23)cc1 ; c1ccc(-c2cccc3c2oc2ccc(-c4ccc(N(c5ccc(-c6ccc7oc8ccccc8c7c6)cc5)c5cccc6c5c5ccccc5n6-c5ccccc5)cc4)cc23)cc1] 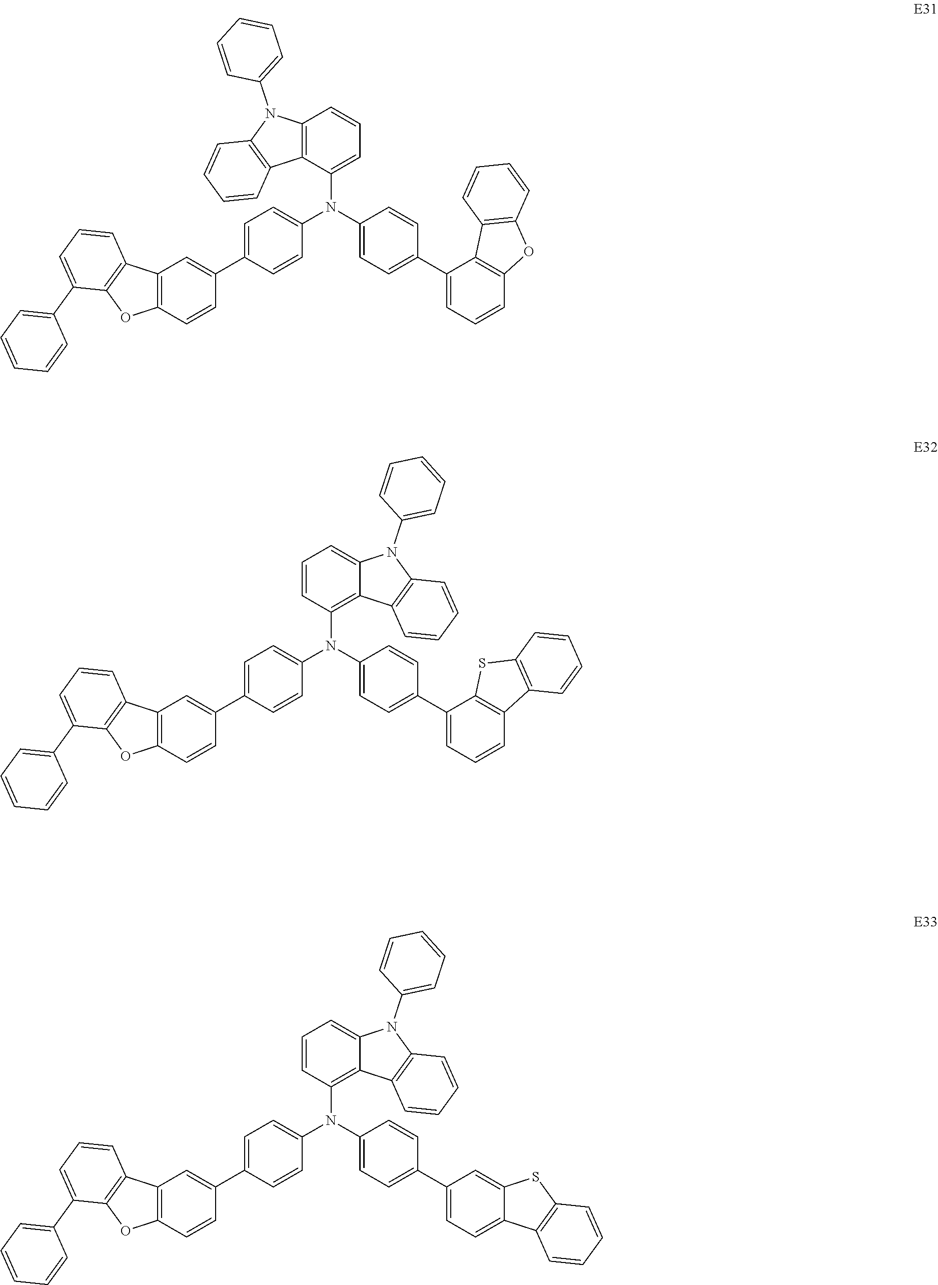 [SMILES: c1ccc(-c2cccc3c2oc2ccc(-c4ccc(N(c5ccc(-c6cccc7oc8ccccc8c67)cc5)c5cccc6c5c5ccccc5n6-c5ccccc5)cc4)cc23)cc1 ; c1ccc(-c2cccc3c2oc2ccc(-c4ccc(N(c5ccc(-c6cccc7c6sc6ccccc67)cc5)c5cccc6c5c5ccccc5n6-c5ccccc5)cc4)cc23)cc1 ; c1ccc(-c2cccc3c2oc2ccc(-c4ccc(N(c5ccc(-c6ccc7c(c6)sc6ccccc67)cc5)c5cccc6c5c5ccccc5n6-c5ccccc5)cc4)cc23)cc1] 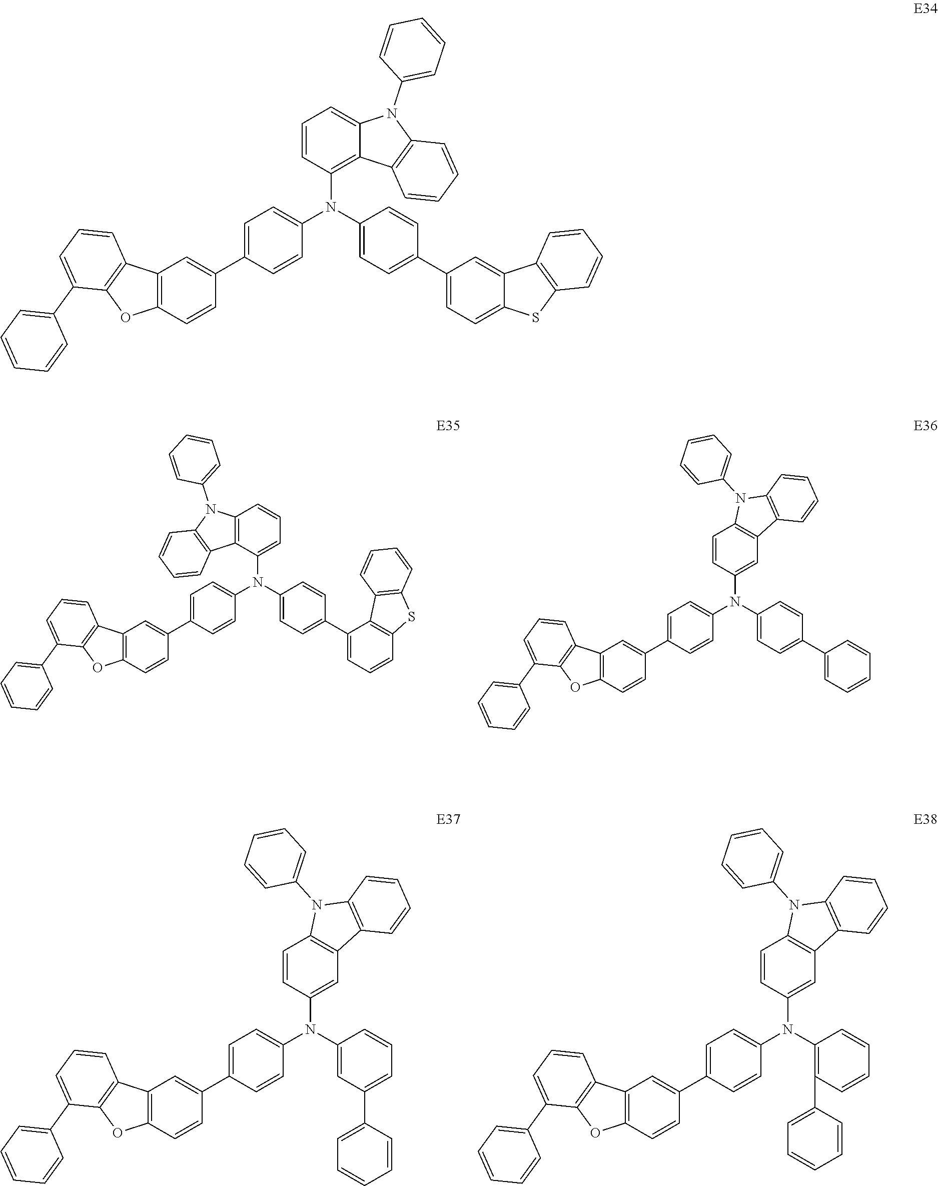 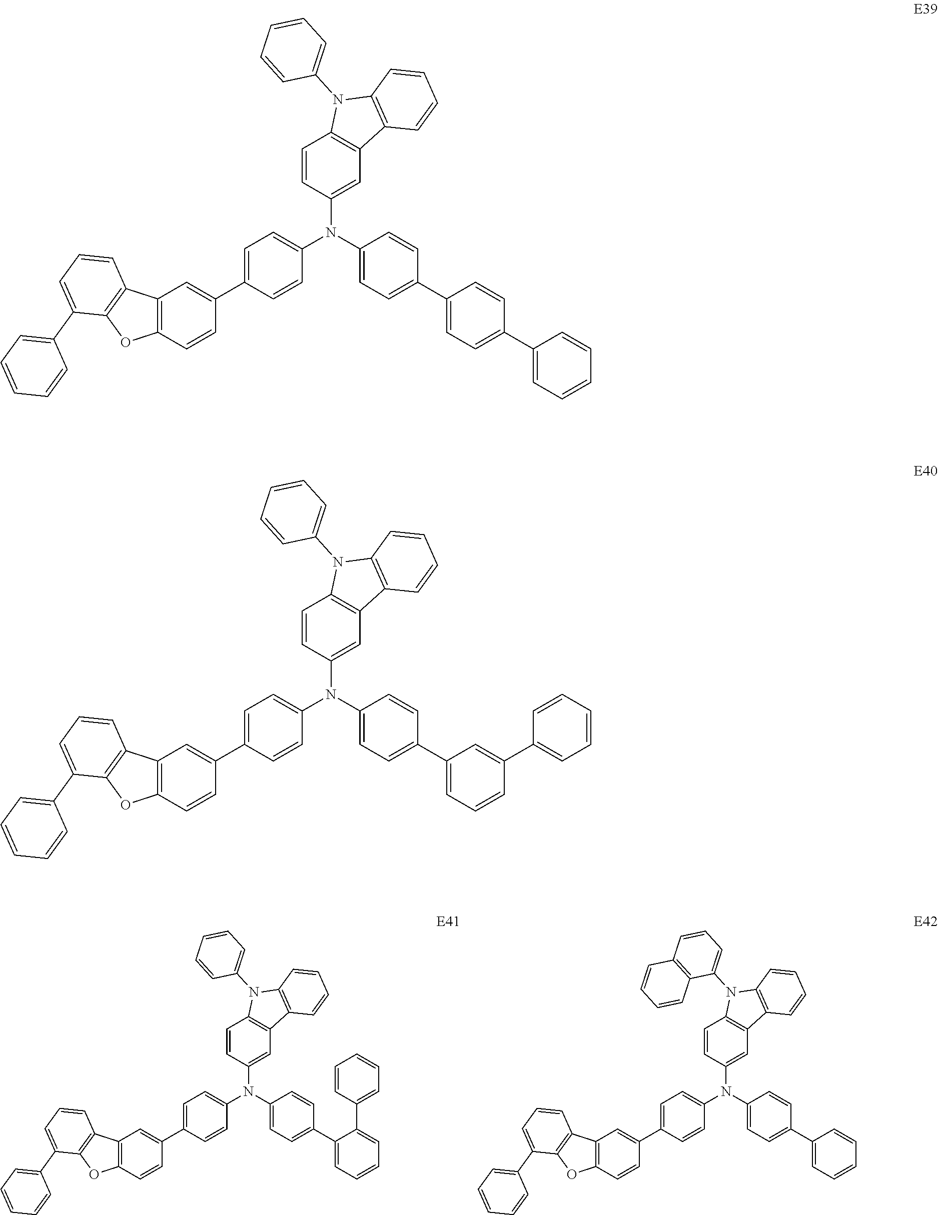 [SMILES: c1ccc(-c2ccc(-c3ccc(N(c4ccc(-c5ccc6oc7c(-c8ccccc8)cccc7c6c5)cc4)c4ccc5c(c4)c4ccccc4n5-c4ccccc4)cc3)cc2)cc1 ; c1ccc(-c2cccc(-c3ccc(N(c4ccc(-c5ccc6oc7c(-c8ccccc8)cccc7c6c5)cc4)c4ccc5c(c4)c4ccccc4n5-c4ccccc4)cc3)c2)cc1 ; c1ccc(-c2ccccc2-c2ccc(N(c3ccc(-c4ccc5oc6c(-c7ccccc7)cccc6c5c4)cc3)c3ccc4c(c3)c3ccccc3n4-c3ccccc3)cc2)cc1 ; c1ccc(-c2ccc(N(c3ccc(-c4ccc5oc6c(-c7ccccc7)cccc6c5c4)cc3)c3ccc4c(c3)c3ccccc3n4-c3cccc4ccccc34)cc2)cc1] 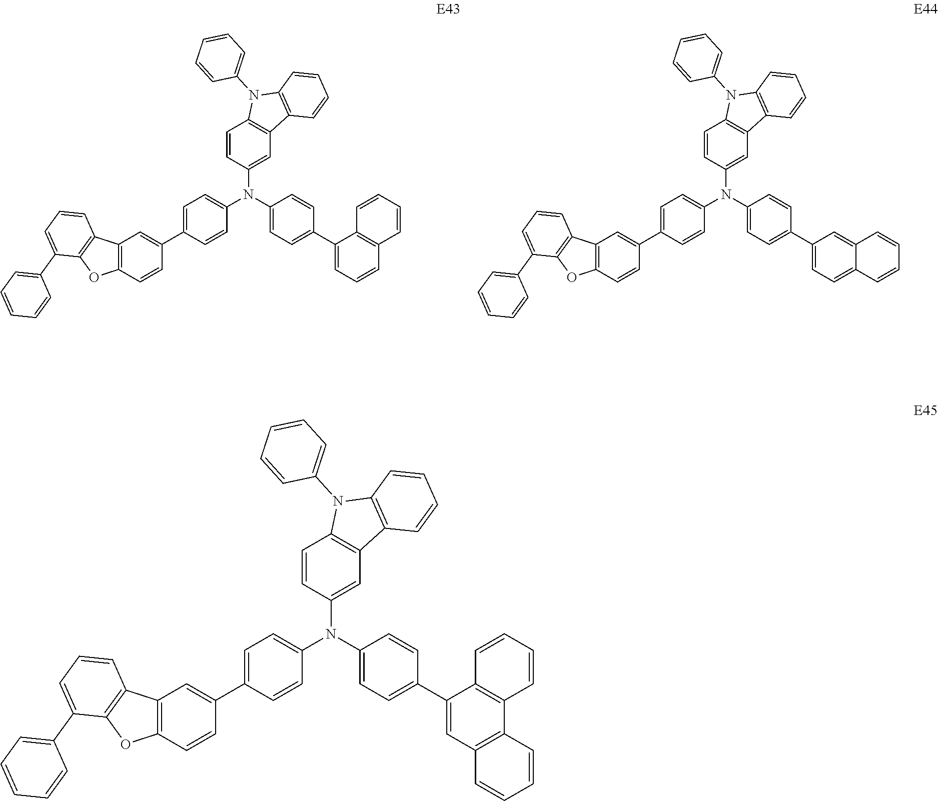 [SMILES: c1ccc(-c2cccc3c2oc2ccc(-c4ccc(N(c5ccc(-c6cccc7ccccc67)cc5)c5ccc6c(c5)c5ccccc5n6-c5ccccc5)cc4)cc23)cc1 ; c1ccc(-c2cccc3c2oc2ccc(-c4ccc(N(c5ccc(-c6ccc7ccccc7c6)cc5)c5ccc6c(c5)c5ccccc5n6-c5ccccc5)cc4)cc23)cc1 ; c1ccc(-c2cccc3c2oc2ccc(-c4ccc(N(c5ccc(-c6cc7ccccc7c7ccccc67)cc5)c5ccc6c(c5)c5ccccc5n6-c5ccccc5)cc4)cc23)cc1] 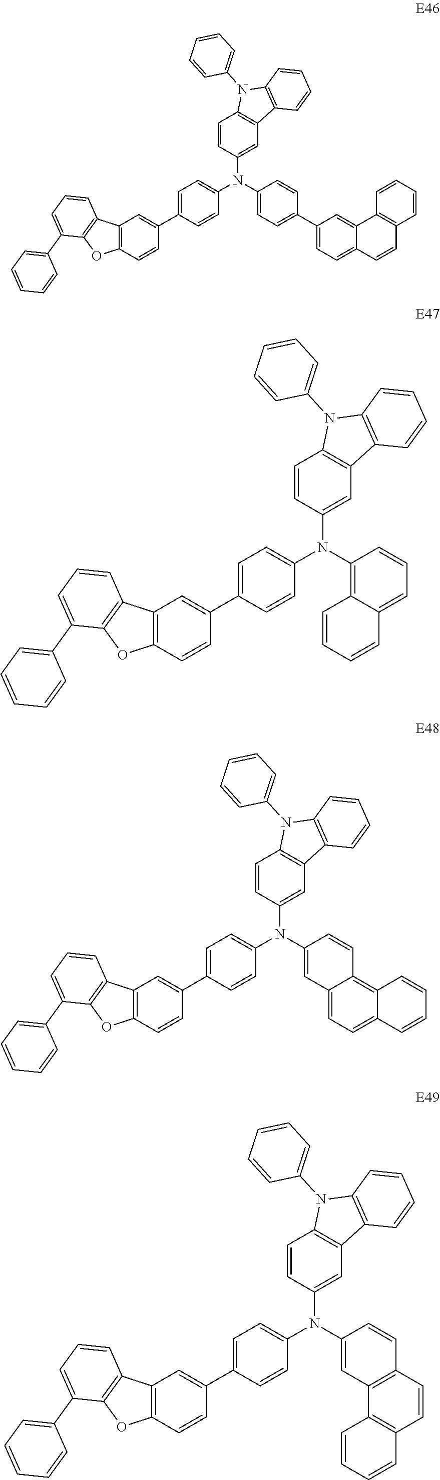 [SMILES: c1ccc(-c2cccc3c2oc2ccc(-c4ccc(N(c5ccc(-c6ccc7ccc8ccccc8c7c6)cc5)c5ccc6c(c5)c5ccccc5n6-c5ccccc5)cc4)cc23)cc1 ; c1ccc(-c2cccc3c2oc2ccc(-c4ccc(N(c5ccc6c(c5)c5ccccc5n6-c5ccccc5)c5cccc6ccccc56)cc4)cc23)cc1 ; c1ccc(-c2cccc3c2oc2ccc(-c4ccc(N(c5ccc6c(ccc7ccccc76)c5)c5ccc6c(c5)c5ccccc5n6-c5ccccc5)cc4)cc23)cc1 ; c1ccc(-c2cccc3c2oc2ccc(-c4ccc(N(c5ccc6ccc7ccccc7c6c5)c5ccc6c(c5)c5ccccc5n6-c5ccccc5)cc4)cc23)cc1] 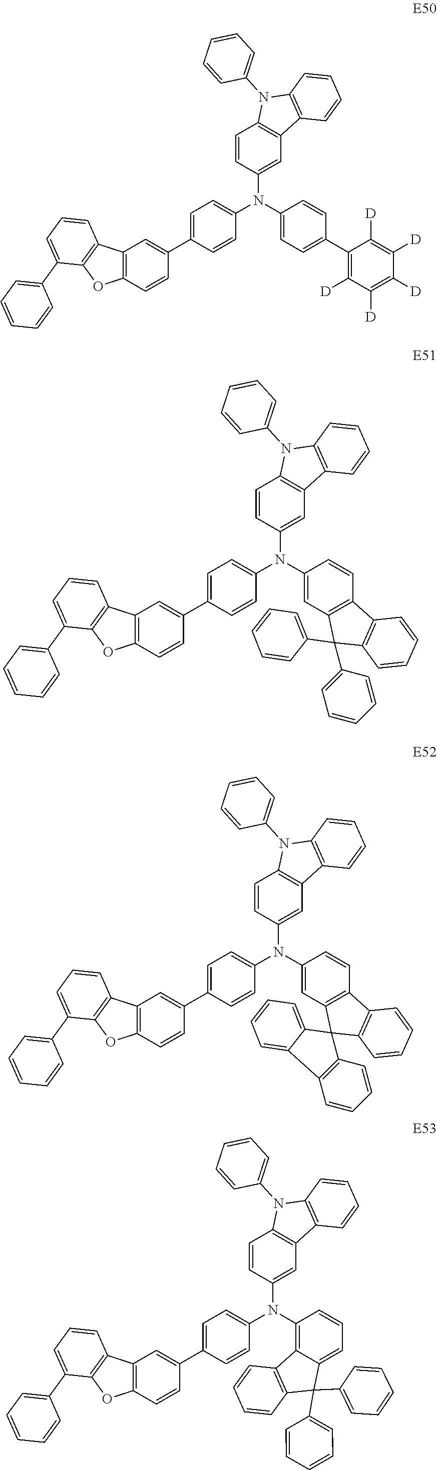 [SMILES: [2H]c1c([2H])c([2H])c(-c2ccc(N(c3ccc(-c4ccc5oc6c(-c7ccccc7)cccc6c5c4)cc3)c3ccc4c(c3)c3ccccc3n4-c3ccccc3)cc2)c([2H])c1[2H] ; c1ccc(-c2cccc3c2oc2ccc(-c4ccc(N(c5ccc6c(c5)C(c5ccccc5)(c5ccccc5)c5ccccc5-6)c5ccc6c(c5)c5ccccc5n6-c5ccccc5)cc4)cc23)cc1 ; c1ccc(-c2cccc3c2oc2ccc(-c4ccc(N(c5ccc6c(c5)C5(c7ccccc7-c7ccccc75)c5ccccc5-6)c5ccc6c(c5)c5ccccc5n6-c5ccccc5)cc4)cc23)cc1 ; c1ccc(-c2cccc3c2oc2ccc(-c4ccc(N(c5ccc6c(c5)c5ccccc5n6-c5ccccc5)c5cccc6c5-c5ccccc5C6(c5ccccc5)c5ccccc5)cc4)cc23)cc1] 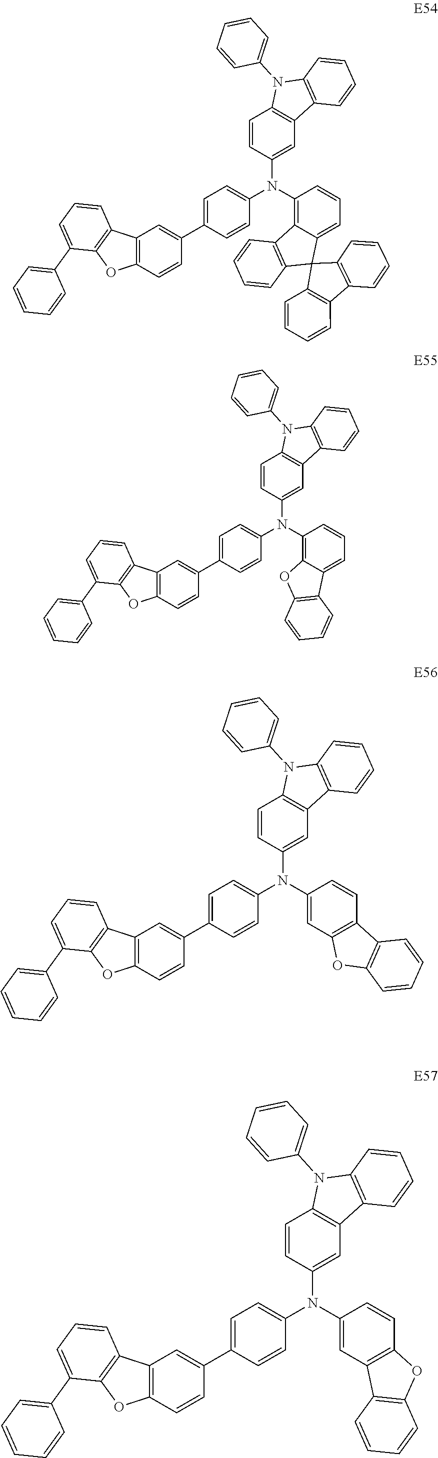 [SMILES: c1ccc(-c2cccc3c2oc2ccc(-c4ccc(N(c5ccc6c(c5)c5ccccc5n6-c5ccccc5)c5cccc6c5-c5ccccc5C65c6ccccc6-c6ccccc65)cc4)cc23)cc1 ; c1ccc(-c2cccc3c2oc2ccc(-c4ccc(N(c5ccc6c(c5)c5ccccc5n6-c5ccccc5)c5cccc6c5oc5ccccc56)cc4)cc23)cc1 ; c1ccc(-c2cccc3c2oc2ccc(-c4ccc(N(c5ccc6c(c5)oc5ccccc56)c5ccc6c(c5)c5ccccc5n6-c5ccccc5)cc4)cc23)cc1 ; c1ccc(-c2cccc3c2oc2ccc(-c4ccc(N(c5ccc6oc7ccccc7c6c5)c5ccc6c(c5)c5ccccc5n6-c5ccccc5)cc4)cc23)cc1] 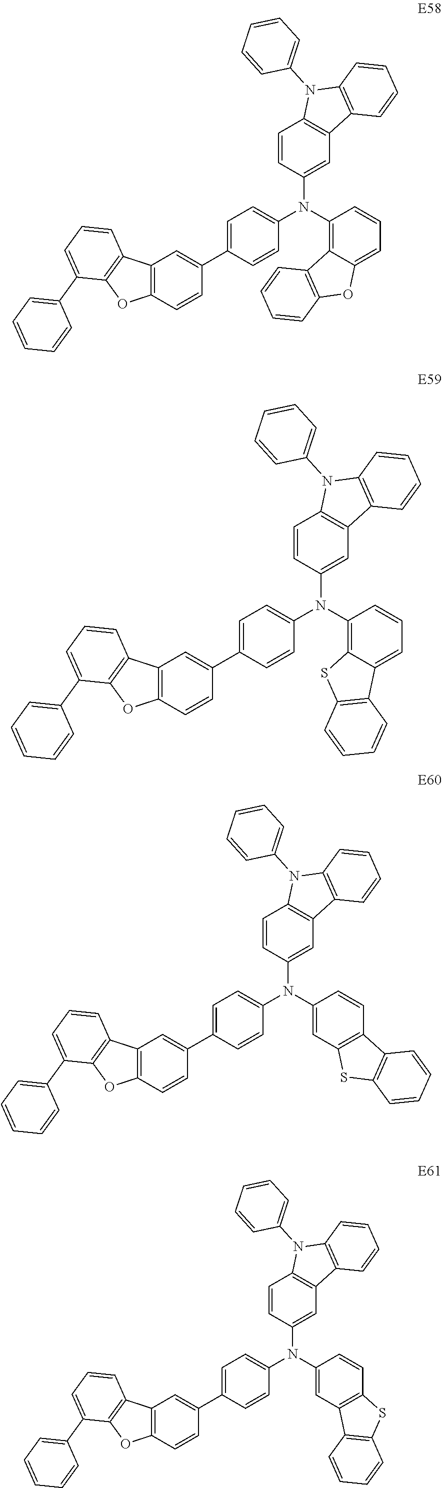 [SMILES: c1ccc(-c2cccc3c2oc2ccc(-c4ccc(N(c5ccc6c(c5)c5ccccc5n6-c5ccccc5)c5cccc6oc7ccccc7c56)cc4)cc23)cc1 ; c1ccc(-c2cccc3c2oc2ccc(-c4ccc(N(c5ccc6c(c5)c5ccccc5n6-c5ccccc5)c5cccc6c5sc5ccccc56)cc4)cc23)cc1 ; c1ccc(-c2cccc3c2oc2ccc(-c4ccc(N(c5ccc6c(c5)sc5ccccc56)c5ccc6c(c5)c5ccccc5n6-c5ccccc5)cc4)cc23)cc1 ; c1ccc(-c2cccc3c2oc2ccc(-c4ccc(N(c5ccc6sc7ccccc7c6c5)c5ccc6c(c5)c5ccccc5n6-c5ccccc5)cc4)cc23)cc1] 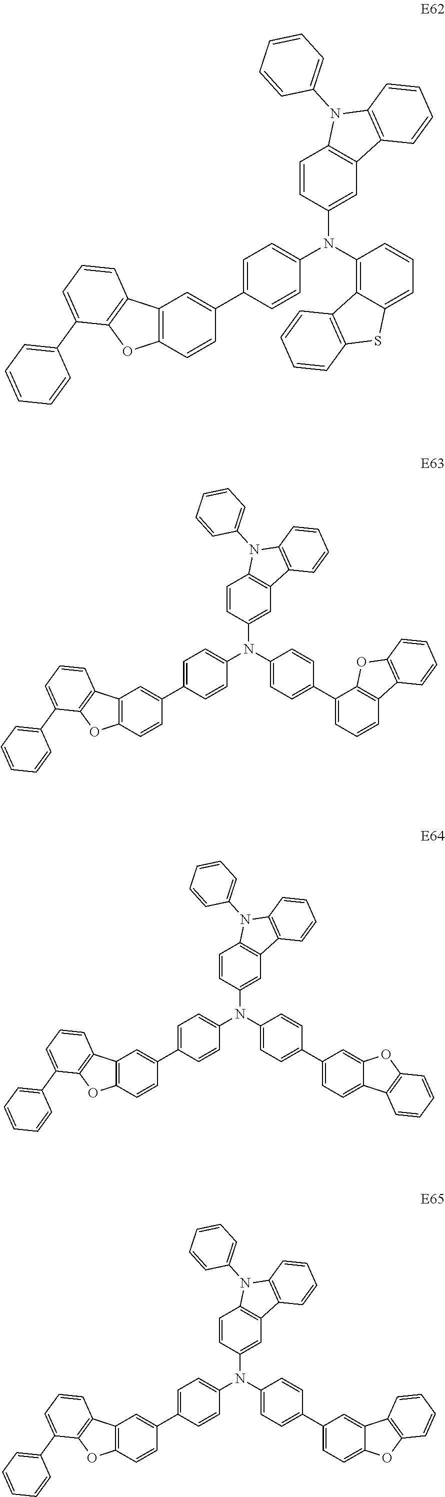 [SMILES: c1ccc(-c2cccc3c2oc2ccc(-c4ccc(N(c5ccc6c(c5)c5ccccc5n6-c5ccccc5)c5cccc6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc6c(c5)c5ccccc5n6-c5ccccc5)cc4)cc23)cc1 ; c1ccc(-c2cccc3c2oc2ccc(-c4ccc(N(c5ccc(-c6ccc7c(c6)oc6ccccc67)cc5)c5ccc6c(c5)c5ccccc5n6-c5ccccc5)cc4)cc23)cc1 ; c1ccc(-c2cccc3c2oc2ccc(-c4ccc(N(c5ccc(-c6ccc7oc8ccccc8c7c6)cc5)c5ccc6c(c5)c5ccccc5n6-c5ccccc5)cc4)cc23)cc1] 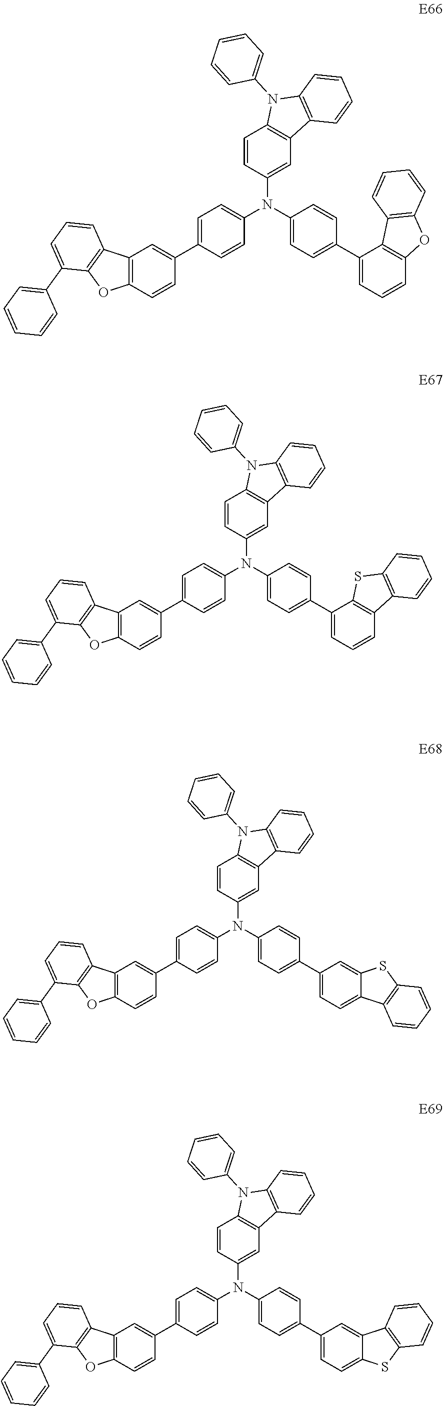 [SMILES: c1ccc(-c2cccc3c2oc2ccc(-c4ccc(N(c5ccc(-c6cccc7oc8ccccc8c67)cc5)c5ccc6c(c5)c5ccccc5n6-c5ccccc5)cc4)cc23)cc1 ; c1ccc(-c2cccc3c2oc2ccc(-c4ccc(N(c5ccc(-c6cccc7c6sc6ccccc67)cc5)c5ccc6c(c5)c5ccccc5n6-c5ccccc5)cc4)cc23)cc1 ; c1ccc(-c2cccc3c2oc2ccc(-c4ccc(N(c5ccc(-c6ccc7c(c6)sc6ccccc67)cc5)c5ccc6c(c5)c5ccccc5n6-c5ccccc5)cc4)cc23)cc1 ; c1ccc(-c2cccc3c2oc2ccc(-c4ccc(N(c5ccc(-c6ccc7sc8ccccc8c7c6)cc5)c5ccc6c(c5)c5ccccc5n6-c5ccccc5)cc4)cc23)cc1] 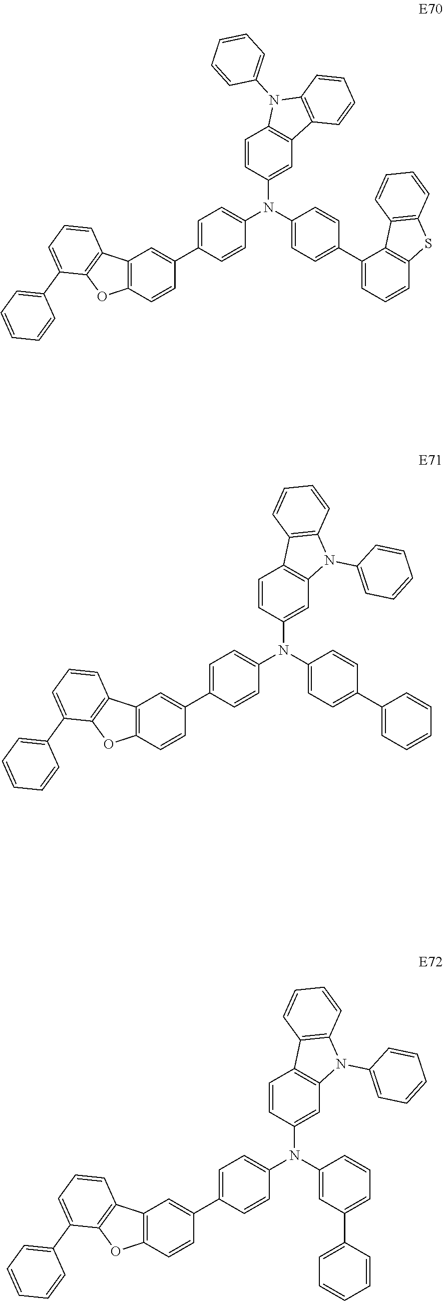 [SMILES: c1ccc(-c2cccc3c2oc2ccc(-c4ccc(N(c5ccc(-c6cccc7sc8ccccc8c67)cc5)c5ccc6c(c5)c5ccccc5n6-c5ccccc5)cc4)cc23)cc1 ; c1ccc(-c2ccc(N(c3ccc(-c4ccc5oc6c(-c7ccccc7)cccc6c5c4)cc3)c3ccc4c5ccccc5n(-c5ccccc5)c4c3)cc2)cc1 ; c1ccc(-c2cccc(N(c3ccc(-c4ccc5oc6c(-c7ccccc7)cccc6c5c4)cc3)c3ccc4c5ccccc5n(-c5ccccc5)c4c3)c2)cc1] 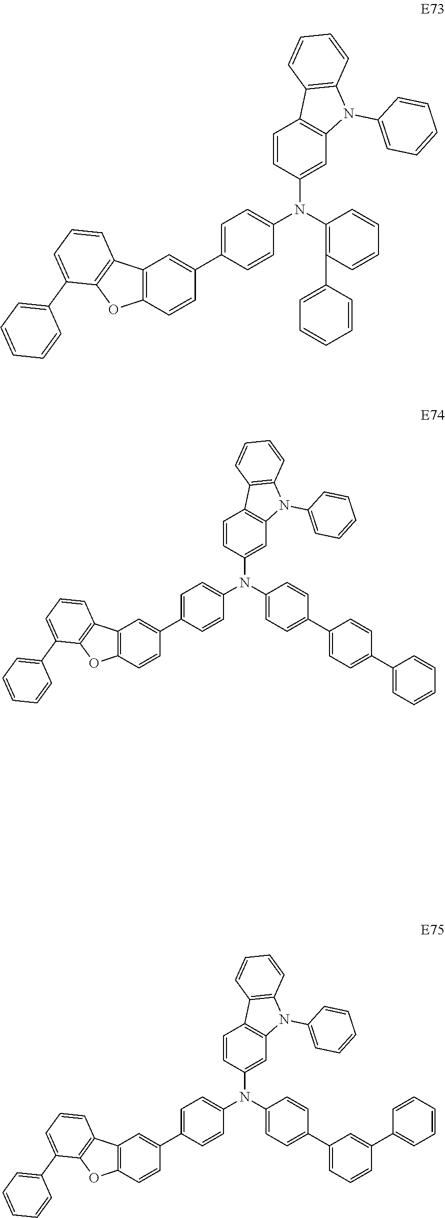 [SMILES: c1ccc(-c2ccccc2N(c2ccc(-c3ccc4oc5c(-c6ccccc6)cccc5c4c3)cc2)c2ccc3c4ccccc4n(-c4ccccc4)c3c2)cc1 ; c1ccc(-c2ccc(-c3ccc(N(c4ccc(-c5ccc6oc7c(-c8ccccc8)cccc7c6c5)cc4)c4ccc5c6ccccc6n(-c6ccccc6)c5c4)cc3)cc2)cc1 ; c1ccc(-c2cccc(-c3ccc(N(c4ccc(-c5ccc6oc7c(-c8ccccc8)cccc7c6c5)cc4)c4ccc5c6ccccc6n(-c6ccccc6)c5c4)cc3)c2)cc1] 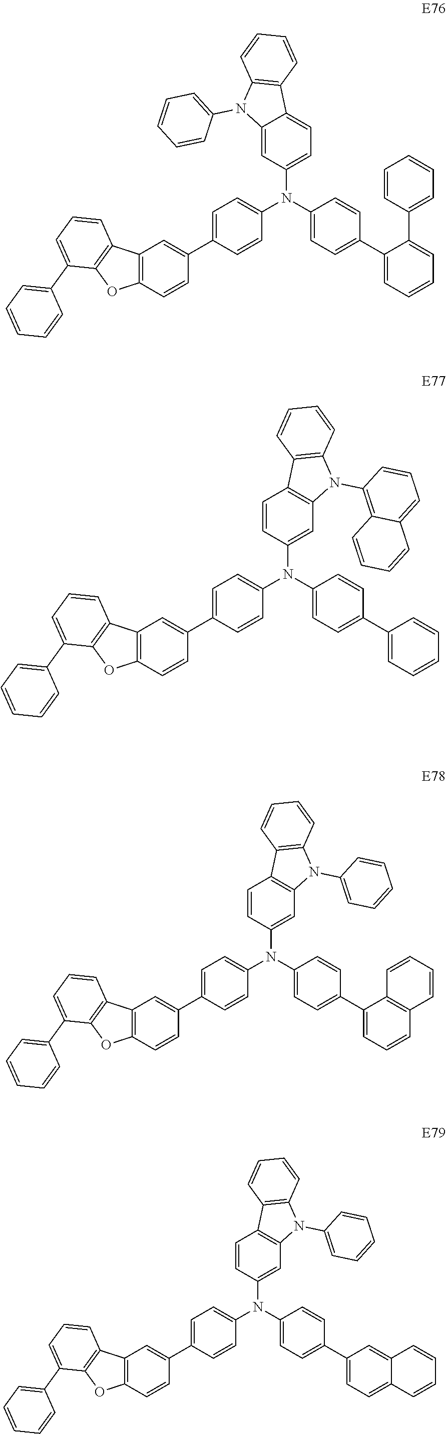 [SMILES: c1ccc(-c2ccccc2-c2ccc(N(c3ccc(-c4ccc5oc6c(-c7ccccc7)cccc6c5c4)cc3)c3ccc4c5ccccc5n(-c5ccccc5)c4c3)cc2)cc1 ; c1ccc(-c2ccc(N(c3ccc(-c4ccc5oc6c(-c7ccccc7)cccc6c5c4)cc3)c3ccc4c5ccccc5n(-c5cccc6ccccc56)c4c3)cc2)cc1 ; c1ccc(-c2cccc3c2oc2ccc(-c4ccc(N(c5ccc(-c6cccc7ccccc67)cc5)c5ccc6c7ccccc7n(-c7ccccc7)c6c5)cc4)cc23)cc1 ; c1ccc(-c2cccc3c2oc2ccc(-c4ccc(N(c5ccc(-c6ccc7ccccc7c6)cc5)c5ccc6c7ccccc7n(-c7ccccc7)c6c5)cc4)cc23)cc1] 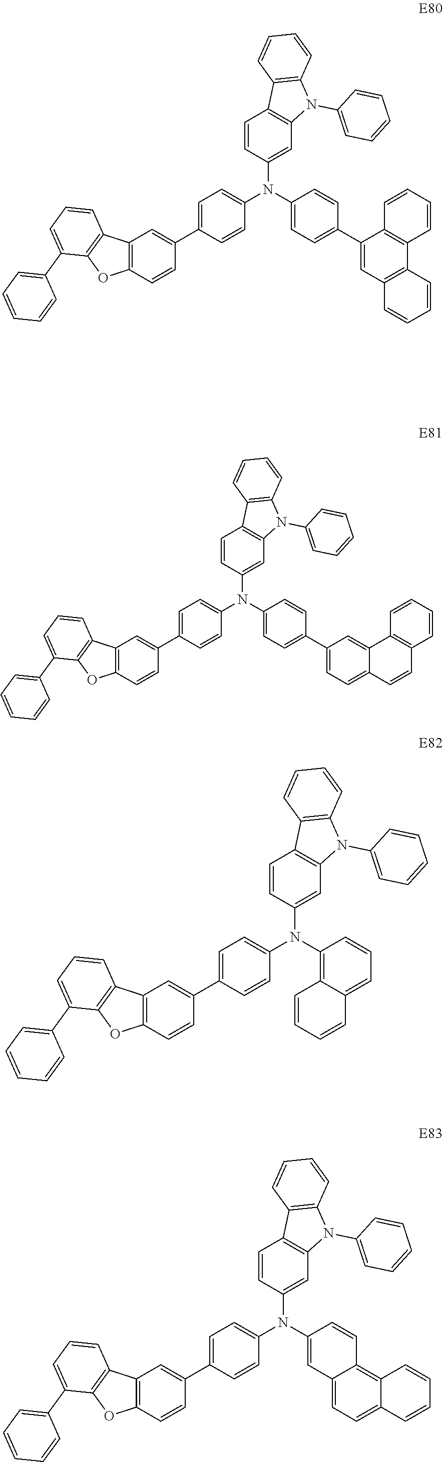 [SMILES: c1ccc(-c2cccc3c2oc2ccc(-c4ccc(N(c5ccc(-c6cc7ccccc7c7ccccc67)cc5)c5ccc6c7ccccc7n(-c7ccccc7)c6c5)cc4)cc23)cc1 ; c1ccc(-c2cccc3c2oc2ccc(-c4ccc(N(c5ccc(-c6ccc7ccc8ccccc8c7c6)cc5)c5ccc6c7ccccc7n(-c7ccccc7)c6c5)cc4)cc23)cc1 ; c1ccc(-c2cccc3c2oc2ccc(-c4ccc(N(c5ccc6c7ccccc7n(-c7ccccc7)c6c5)c5cccc6ccccc56)cc4)cc23)cc1 ; c1ccc(-c2cccc3c2oc2ccc(-c4ccc(N(c5ccc6c(ccc7ccccc76)c5)c5ccc6c7ccccc7n(-c7ccccc7)c6c5)cc4)cc23)cc1] 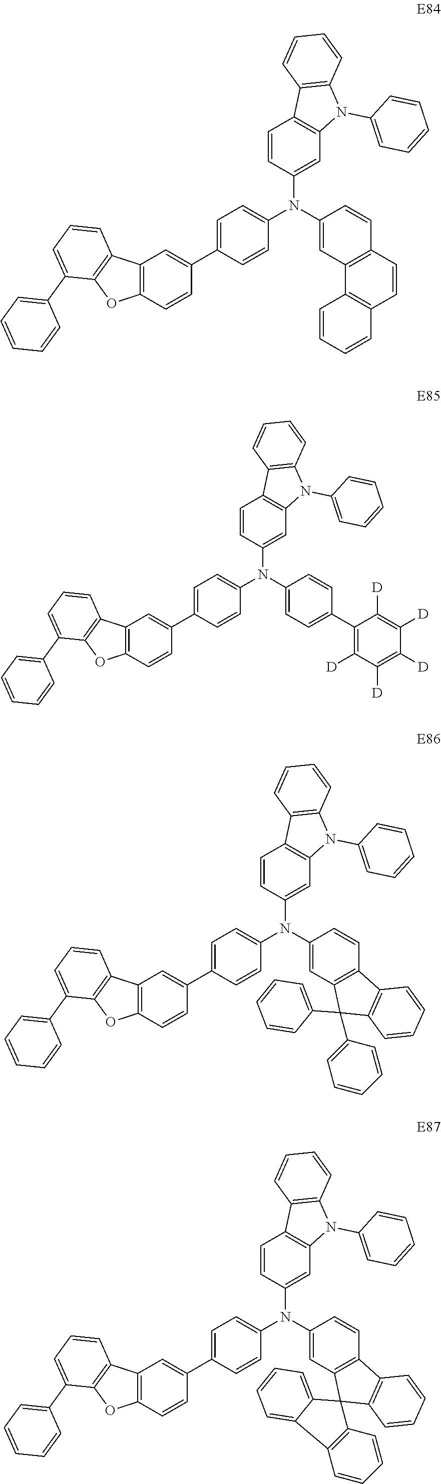 [SMILES: c1ccc(-c2cccc3c2oc2ccc(-c4ccc(N(c5ccc6ccc7ccccc7c6c5)c5ccc6c7ccccc7n(-c7ccccc7)c6c5)cc4)cc23)cc1 ; [2H]c1c([2H])c([2H])c(-c2ccc(N(c3ccc(-c4ccc5oc6c(-c7ccccc7)cccc6c5c4)cc3)c3ccc4c5ccccc5n(-c5ccccc5)c4c3)cc2)c([2H])c1[2H] ; c1ccc(-c2cccc3c2oc2ccc(-c4ccc(N(c5ccc6c(c5)C(c5ccccc5)(c5ccccc5)c5ccccc5-6)c5ccc6c7ccccc7n(-c7ccccc7)c6c5)cc4)cc23)cc1 ; c1ccc(-c2cccc3c2oc2ccc(-c4ccc(N(c5ccc6c(c5)C5(c7ccccc7-c7ccccc75)c5ccccc5-6)c5ccc6c7ccccc7n(-c7ccccc7)c6c5)cc4)cc23)cc1] 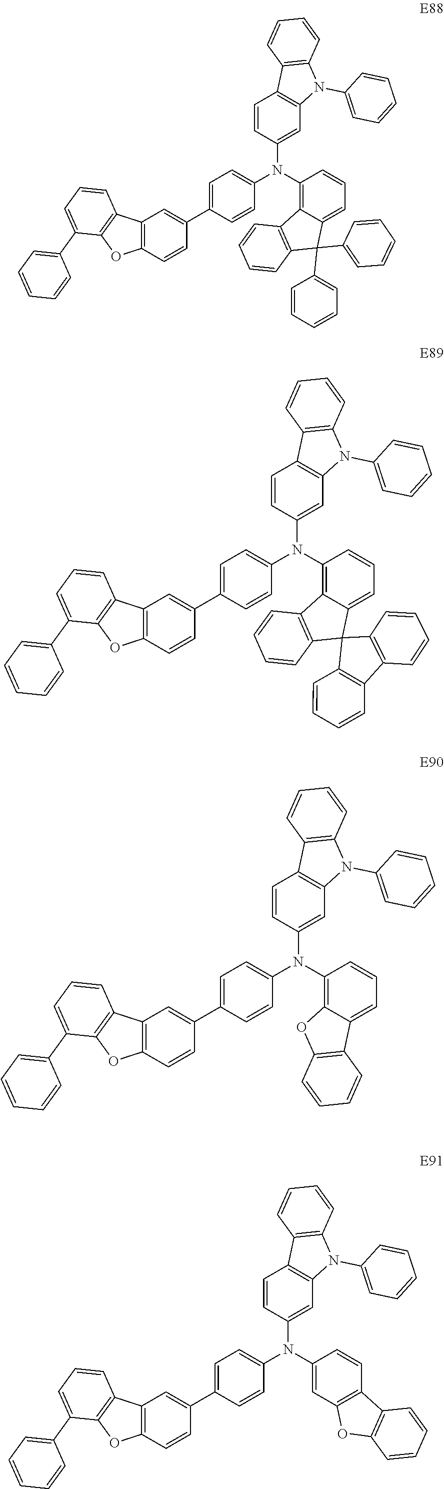 [SMILES: c1ccc(-c2cccc3c2oc2ccc(-c4ccc(N(c5ccc6c7ccccc7n(-c7ccccc7)c6c5)c5cccc6c5-c5ccccc5C6(c5ccccc5)c5ccccc5)cc4)cc23)cc1 ; c1ccc(-c2cccc3c2oc2ccc(-c4ccc(N(c5ccc6c7ccccc7n(-c7ccccc7)c6c5)c5cccc6c5-c5ccccc5C65c6ccccc6-c6ccccc65)cc4)cc23)cc1 ; c1ccc(-c2cccc3c2oc2ccc(-c4ccc(N(c5ccc6c7ccccc7n(-c7ccccc7)c6c5)c5cccc6c5oc5ccccc56)cc4)cc23)cc1 ; c1ccc(-c2cccc3c2oc2ccc(-c4ccc(N(c5ccc6c(c5)oc5ccccc56)c5ccc6c7ccccc7n(-c7ccccc7)c6c5)cc4)cc23)cc1] 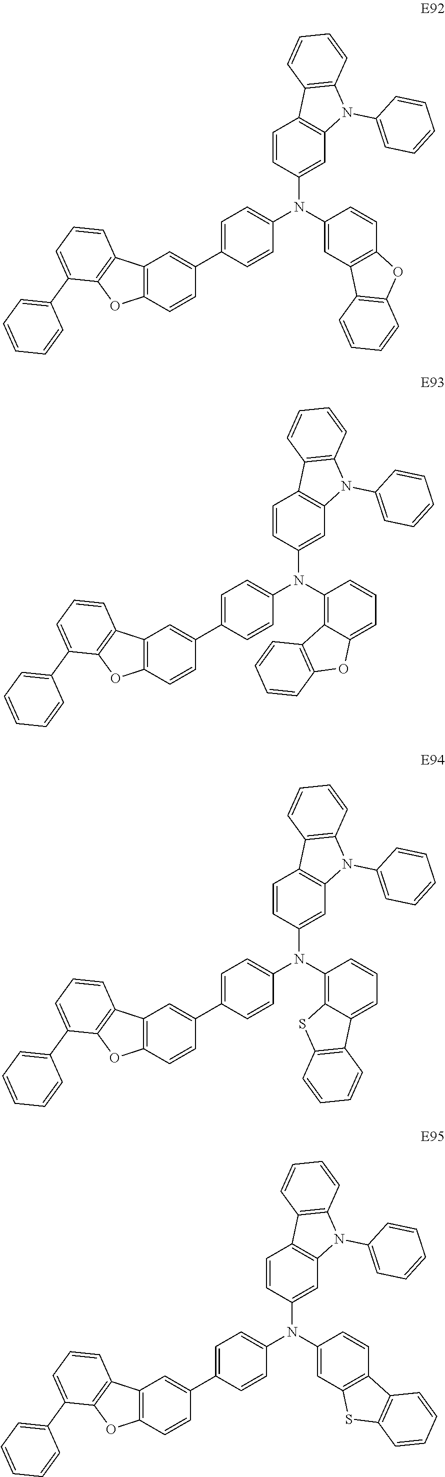 [SMILES: c1ccc(-c2cccc3c2oc2ccc(-c4ccc(N(c5ccc6oc7ccccc7c6c5)c5ccc6c7ccccc7n(-c7ccccc7)c6c5)cc4)cc23)cc1 ; c1ccc(-c2cccc3c2oc2ccc(-c4ccc(N(c5ccc6c7ccccc7n(-c7ccccc7)c6c5)c5cccc6oc7ccccc7c56)cc4)cc23)cc1 ; c1ccc(-c2cccc3c2oc2ccc(-c4ccc(N(c5ccc6c7ccccc7n(-c7ccccc7)c6c5)c5cccc6c5sc5ccccc56)cc4)cc23)cc1 ; c1ccc(-c2cccc3c2oc2ccc(-c4ccc(N(c5ccc6c(c5)sc5ccccc56)c5ccc6c7ccccc7n(-c7ccccc7)c6c5)cc4)cc23)cc1] 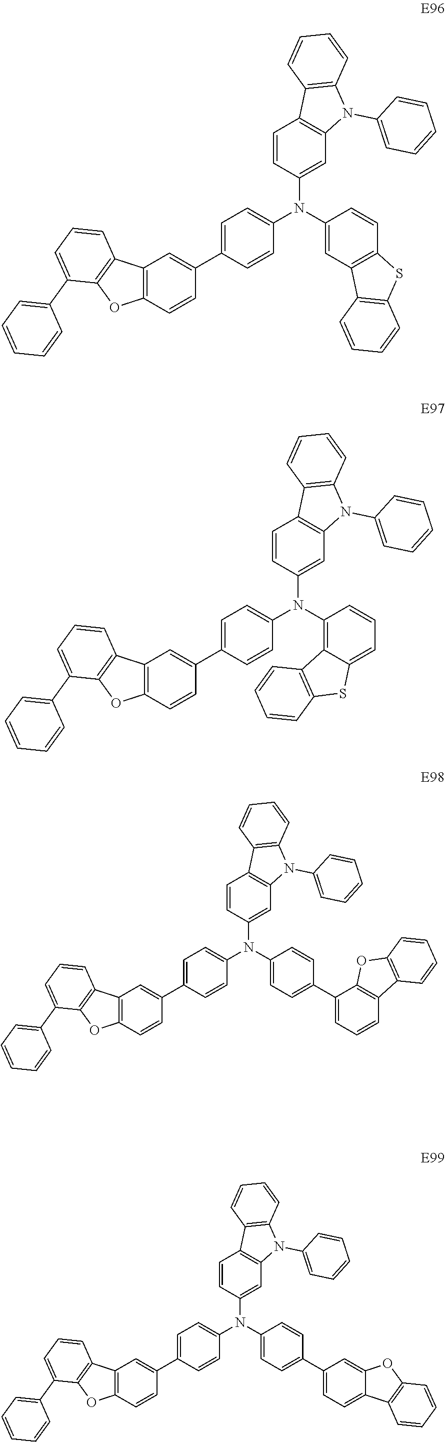 [SMILES: c1ccc(-c2cccc3c2oc2ccc(-c4ccc(N(c5ccc6sc7ccccc7c6c5)c5ccc6c7ccccc7n(-c7ccccc7)c6c5)cc4)cc23)cc1 ; c1ccc(-c2cccc3c2oc2ccc(-c4ccc(N(c5ccc6c7ccccc7n(-c7ccccc7)c6c5)c5cccc6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc6c7ccccc7n(-c7ccccc7)c6c5)cc4)cc23)cc1 ; c1ccc(-c2cccc3c2oc2ccc(-c4ccc(N(c5ccc(-c6ccc7c(c6)oc6ccccc67)cc5)c5ccc6c7ccccc7n(-c7ccccc7)c6c5)cc4)cc23)cc1] 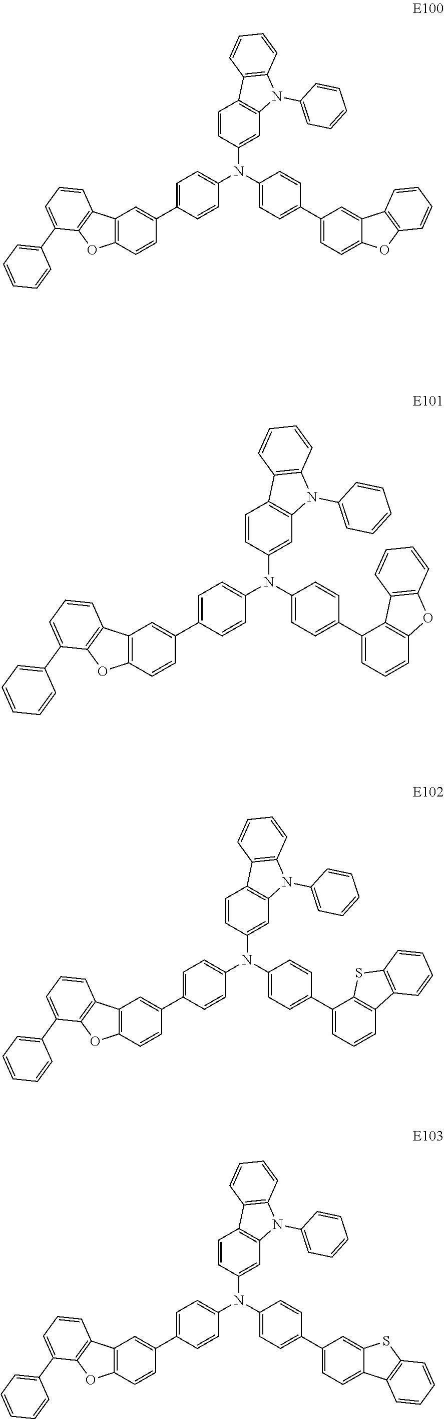 [SMILES: c1ccc(-c2cccc3c2oc2ccc(-c4ccc(N(c5ccc(-c6ccc7oc8ccccc8c7c6)cc5)c5ccc6c7ccccc7n(-c7ccccc7)c6c5)cc4)cc23)cc1 ; c1ccc(-c2cccc3c2oc2ccc(-c4ccc(N(c5ccc(-c6cccc7oc8ccccc8c67)cc5)c5ccc6c7ccccc7n(-c7ccccc7)c6c5)cc4)cc23)cc1 ; c1ccc(-c2cccc3c2oc2ccc(-c4ccc(N(c5ccc(-c6cccc7c6sc6ccccc67)cc5)c5ccc6c7ccccc7n(-c7ccccc7)c6c5)cc4)cc23)cc1 ; c1ccc(-c2cccc3c2oc2ccc(-c4ccc(N(c5ccc(-c6ccc7c(c6)sc6ccccc67)cc5)c5ccc6c7ccccc7n(-c7ccccc7)c6c5)cc4)cc23)cc1] 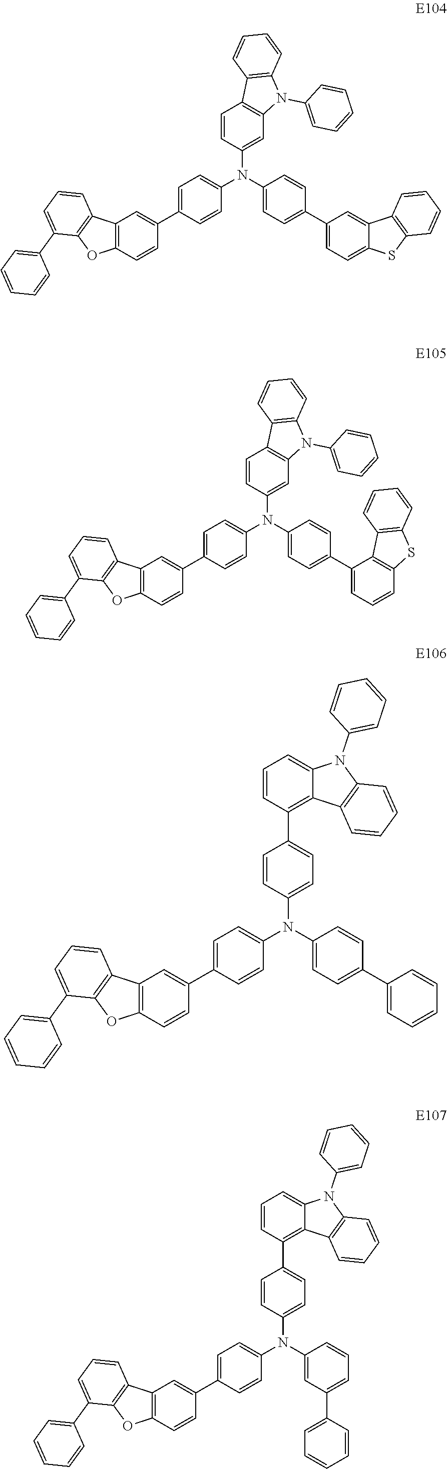 [SMILES: c1ccc(-c2cccc3c2oc2ccc(-c4ccc(N(c5ccc(-c6ccc7sc8ccccc8c7c6)cc5)c5ccc6c7ccccc7n(-c7ccccc7)c6c5)cc4)cc23)cc1 ; c1ccc(-c2cccc3c2oc2ccc(-c4ccc(N(c5ccc(-c6cccc7sc8ccccc8c67)cc5)c5ccc6c7ccccc7n(-c7ccccc7)c6c5)cc4)cc23)cc1 ; c1ccc(-c2ccc(N(c3ccc(-c4ccc5oc6c(-c7ccccc7)cccc6c5c4)cc3)c3ccc(-c4cccc5c4c4ccccc4n5-c4ccccc4)cc3)cc2)cc1 ; c1ccc(-c2cccc(N(c3ccc(-c4ccc5oc6c(-c7ccccc7)cccc6c5c4)cc3)c3ccc(-c4cccc5c4c4ccccc4n5-c4ccccc4)cc3)c2)cc1] 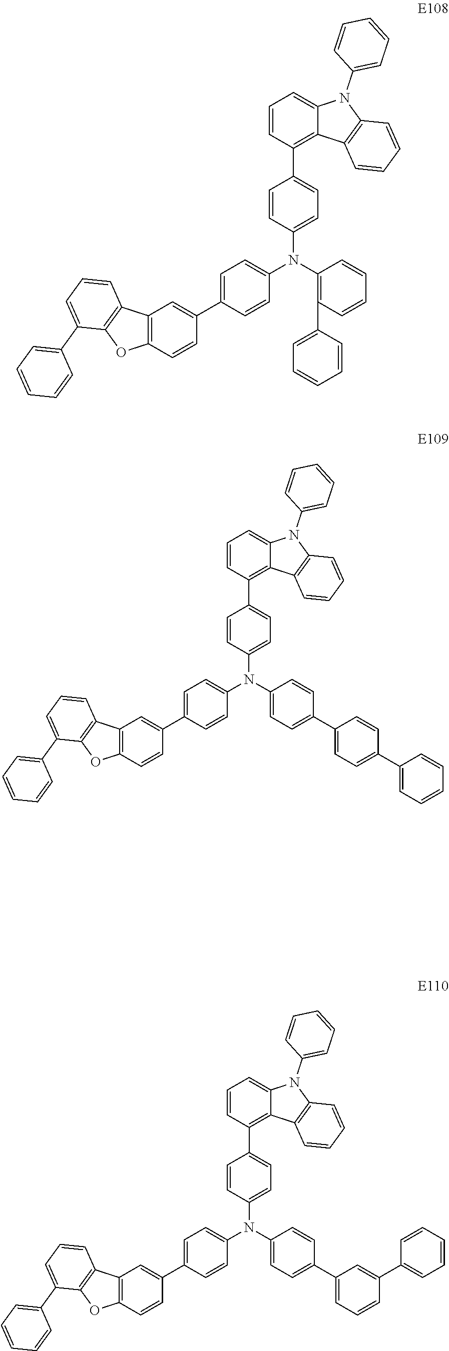 [SMILES: c1ccc(-c2ccccc2N(c2ccc(-c3ccc4oc5c(-c6ccccc6)cccc5c4c3)cc2)c2ccc(-c3cccc4c3c3ccccc3n4-c3ccccc3)cc2)cc1 ; c1ccc(-c2ccc(-c3ccc(N(c4ccc(-c5ccc6oc7c(-c8ccccc8)cccc7c6c5)cc4)c4ccc(-c5cccc6c5c5ccccc5n6-c5ccccc5)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5cccc6c5c5ccccc5n6-c5ccccc5)cc4)cc3)c2)cc1] 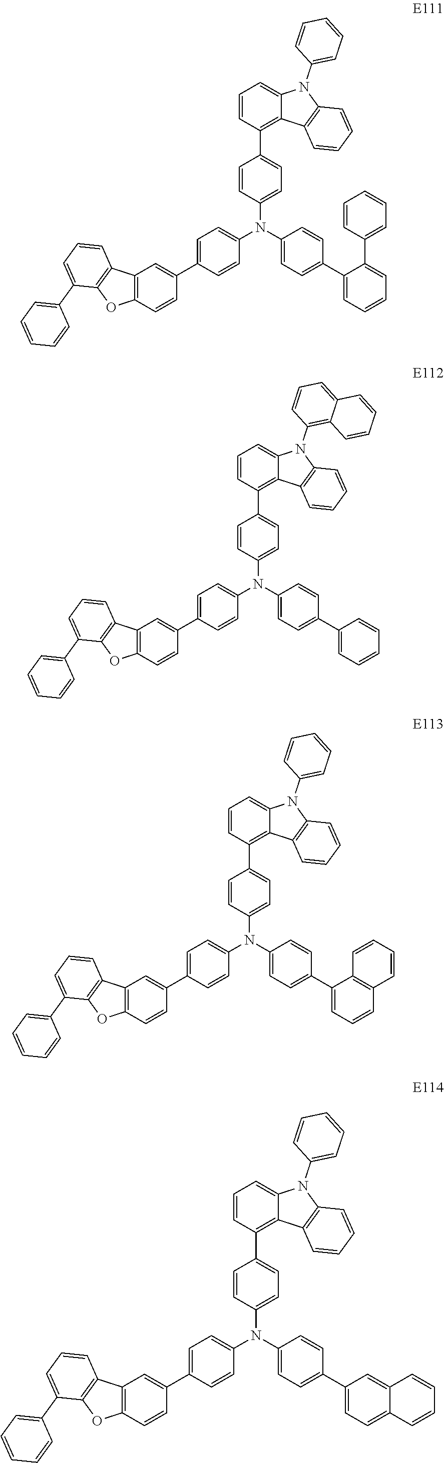 [SMILES: c1ccc(-c2ccccc2-c2ccc(N(c3ccc(-c4ccc5oc6c(-c7ccccc7)cccc6c5c4)cc3)c3ccc(-c4cccc5c4c4ccccc4n5-c4ccccc4)cc3)cc2)cc1 ; c1ccc(-c2ccc(N(c3ccc(-c4ccc5oc6c(-c7ccccc7)cccc6c5c4)cc3)c3ccc(-c4cccc5c4c4ccccc4n5-c4cccc5ccccc45)cc3)cc2)cc1 ; c1ccc(-c2cccc3c2oc2ccc(-c4ccc(N(c5ccc(-c6cccc7ccccc67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6ccc7ccccc7c6)cc5)c5ccc(-c6cccc7c6c6ccccc6n7-c6ccccc6)cc5)cc4)cc23)cc1] 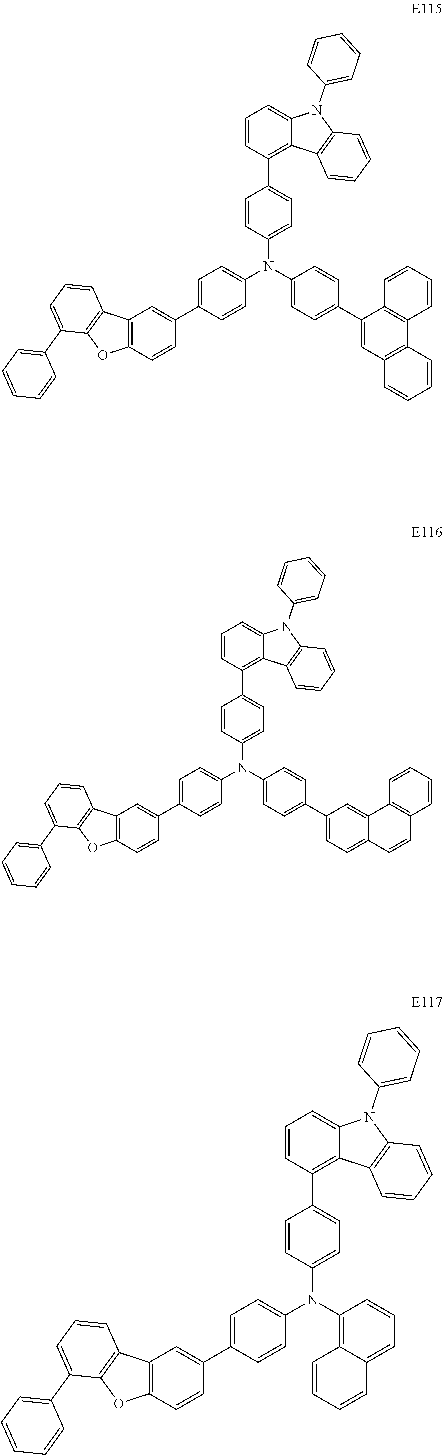 [SMILES: c1ccc(-c2cccc3c2oc2ccc(-c4ccc(N(c5ccc(-c6cc7ccccc7c7ccccc67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6cccc7c6c6ccccc6n7-c6ccccc6)cc5)c5cccc6ccccc56)cc4)cc23)cc1] 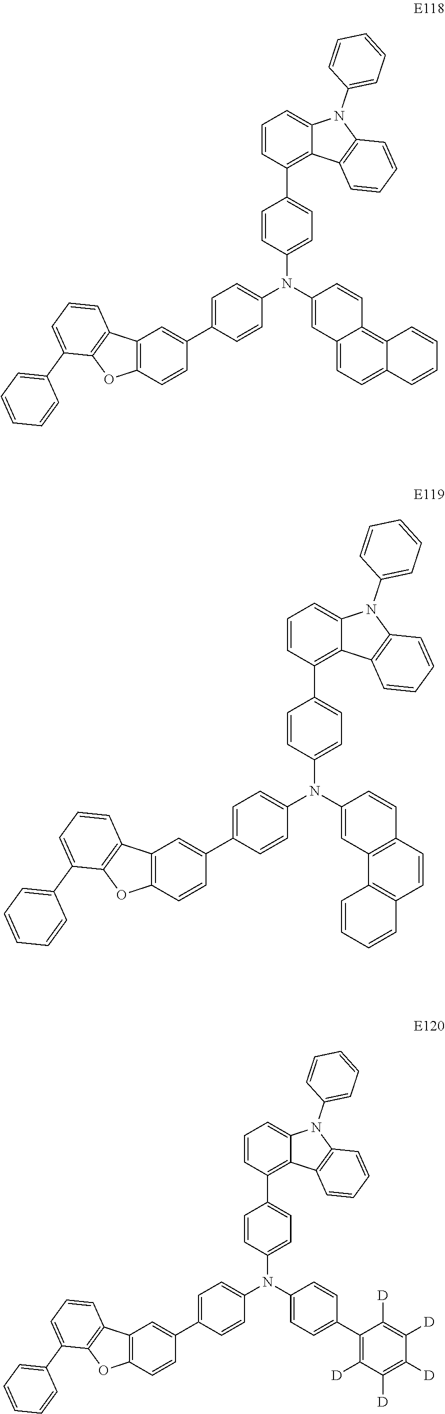 [SMILES: c1ccc(-c2cccc3c2oc2ccc(-c4ccc(N(c5ccc(-c6cccc7c6c6ccccc6n7-c6ccccc6)cc5)c5ccc6c(ccc7ccccc76)c5)cc4)cc23)cc1 ; c1ccc(-c2cccc3c2oc2ccc(-c4ccc(N(c5ccc(-c6cccc7c6c6ccccc6n7-c6ccccc6)cc5)c5ccc6ccc7ccccc7c6c5)cc4)cc23)cc1 ; [2H]c1c([2H])c([2H])c(-c2ccc(N(c3ccc(-c4ccc5oc6c(-c7ccccc7)cccc6c5c4)cc3)c3ccc(-c4cccc5c4c4ccccc4n5-c4ccccc4)cc3)cc2)c([2H])c1[2H]] 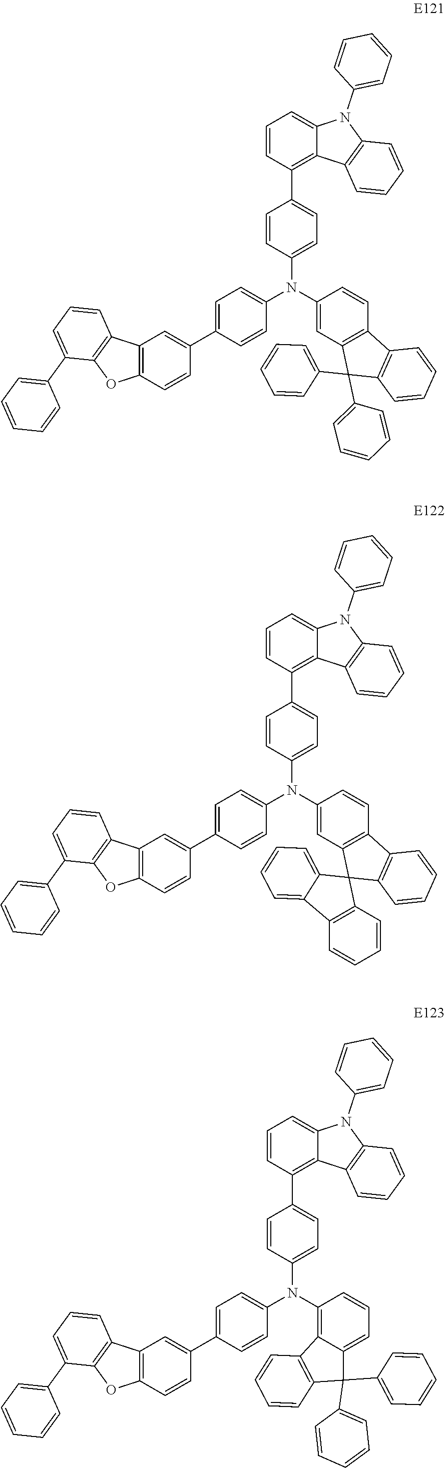 [SMILES: c1ccc(-c2cccc3c2oc2ccc(-c4ccc(N(c5ccc(-c6cccc7c6c6ccccc6n7-c6ccccc6)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6cccc7c6c6ccccc6n7-c6ccccc6)cc5)c5ccc6c(c5)C5(c7ccccc7-c7ccccc75)c5ccccc5-6)cc4)cc23)cc1 ; c1ccc(-c2cccc3c2oc2ccc(-c4ccc(N(c5ccc(-c6cccc7c6c6ccccc6n7-c6ccccc6)cc5)c5cccc6c5-c5ccccc5C6(c5ccccc5)c5ccccc5)cc4)cc23)cc1] 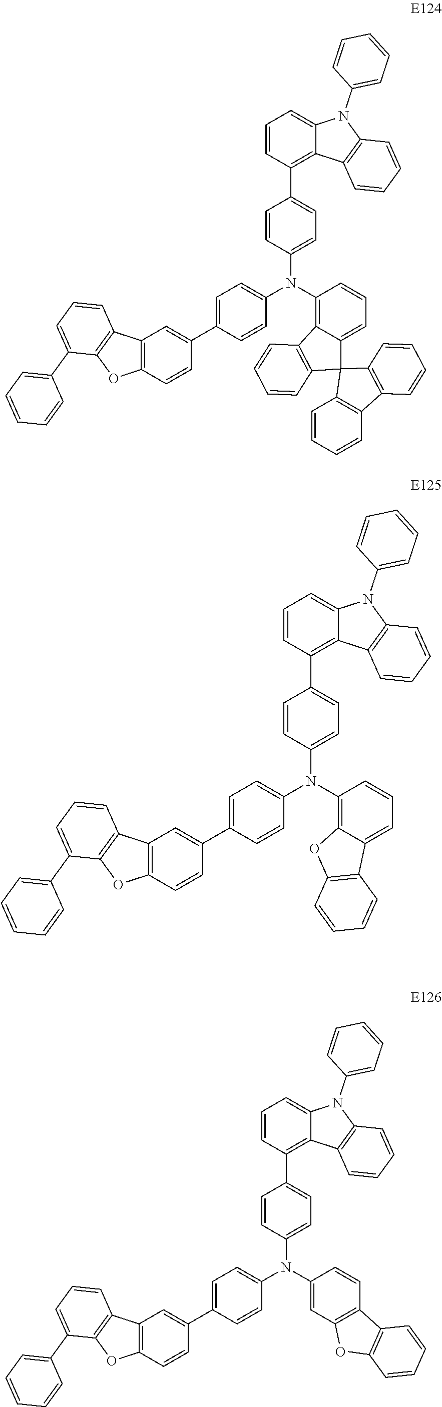 [SMILES: c1ccc(-c2cccc3c2oc2ccc(-c4ccc(N(c5ccc(-c6cccc7c6c6ccccc6n7-c6ccccc6)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6cccc7c6c6ccccc6n7-c6ccccc6)cc5)c5cccc6c5oc5ccccc56)cc4)cc23)cc1 ; c1ccc(-c2cccc3c2oc2ccc(-c4ccc(N(c5ccc(-c6cccc7c6c6ccccc6n7-c6ccccc6)cc5)c5ccc6c(c5)oc5ccccc56)cc4)cc23)cc1] 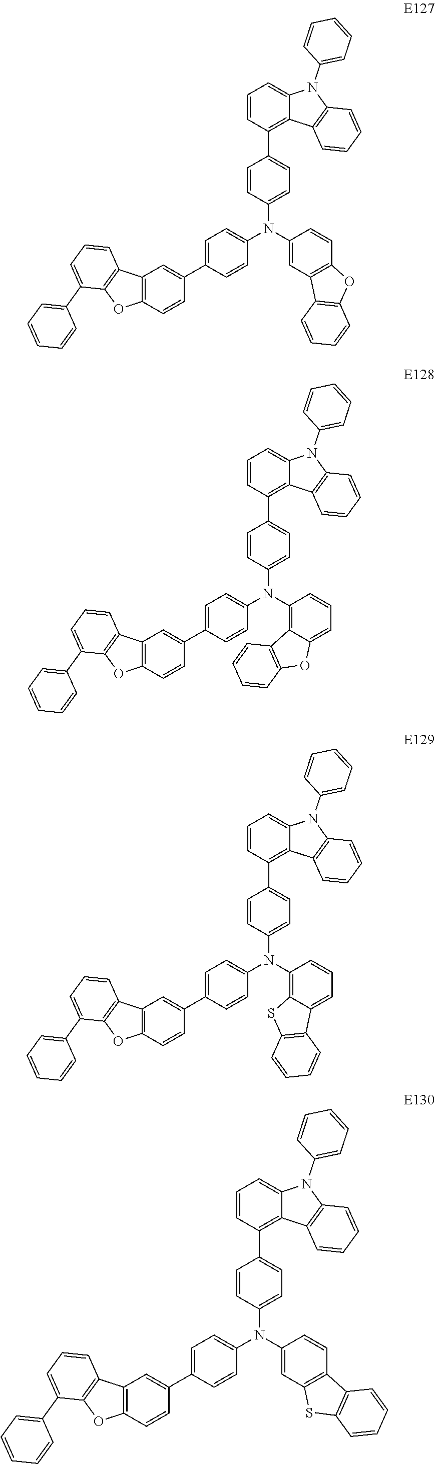 [SMILES: c1ccc(-c2cccc3c2oc2ccc(-c4ccc(N(c5ccc(-c6cccc7c6c6ccccc6n7-c6ccccc6)cc5)c5ccc6oc7ccccc7c6c5)cc4)cc23)cc1 ; c1ccc(-c2cccc3c2oc2ccc(-c4ccc(N(c5ccc(-c6cccc7c6c6ccccc6n7-c6ccccc6)cc5)c5cccc6oc7ccccc7c56)cc4)cc23)cc1 ; c1ccc(-c2cccc3c2oc2ccc(-c4ccc(N(c5ccc(-c6cccc7c6c6ccccc6n7-c6ccccc6)cc5)c5cccc6c5sc5ccccc56)cc4)cc23)cc1 ; c1ccc(-c2cccc3c2oc2ccc(-c4ccc(N(c5ccc(-c6cccc7c6c6ccccc6n7-c6ccccc6)cc5)c5ccc6c(c5)sc5ccccc56)cc4)cc23)cc1] 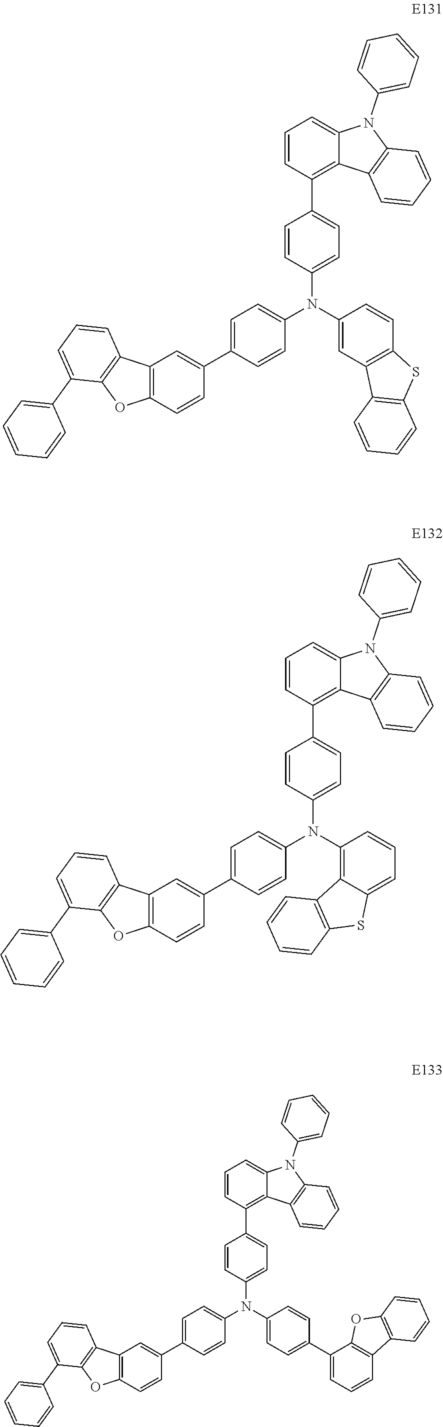 [SMILES: c1ccc(-c2cccc3c2oc2ccc(-c4ccc(N(c5ccc(-c6cccc7c6c6ccccc6n7-c6ccccc6)cc5)c5ccc6sc7ccccc7c6c5)cc4)cc23)cc1 ; c1ccc(-c2cccc3c2oc2ccc(-c4ccc(N(c5ccc(-c6cccc7c6c6ccccc6n7-c6ccccc6)cc5)c5cccc6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc(-c6cccc7c6c6ccccc6n7-c6ccccc6)cc5)cc4)cc23)cc1] 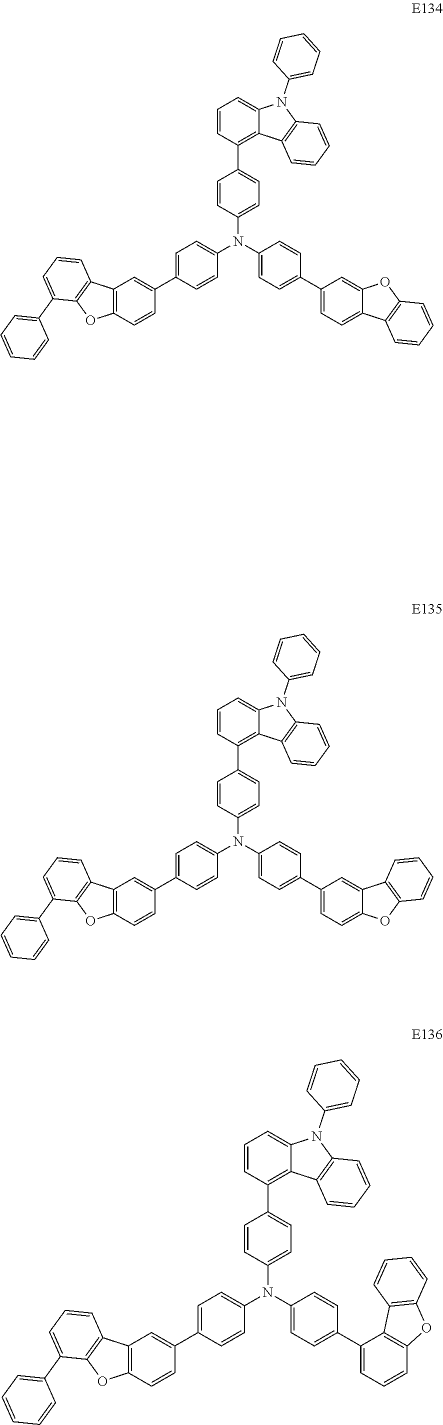 [SMILES: c1ccc(-c2cccc3c2oc2ccc(-c4ccc(N(c5ccc(-c6ccc7c(c6)oc6ccccc67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6ccc7oc8ccccc8c7c6)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6cccc7oc8ccccc8c67)cc5)c5ccc(-c6cccc7c6c6ccccc6n7-c6ccccc6)cc5)cc4)cc23)cc1] 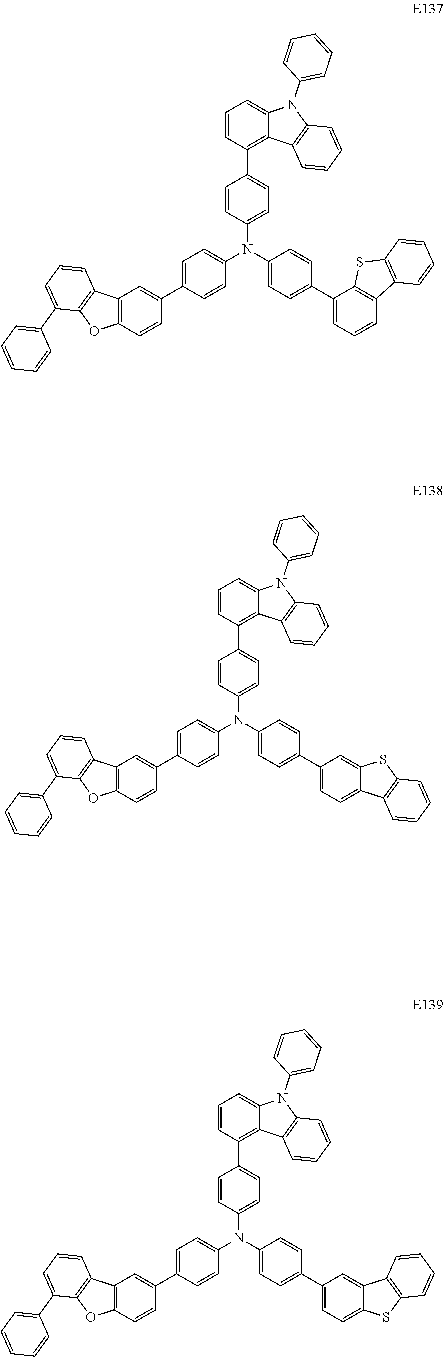 [SMILES: c1ccc(-c2cccc3c2oc2ccc(-c4ccc(N(c5ccc(-c6cccc7c6sc6ccccc67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6cccc7c6c6ccccc6n7-c6ccccc6)cc5)cc4)cc23)cc1 ; c1ccc(-c2cccc3c2oc2ccc(-c4ccc(N(c5ccc(-c6ccc7sc8ccccc8c7c6)cc5)c5ccc(-c6cccc7c6c6ccccc6n7-c6ccccc6)cc5)cc4)cc23)cc1] 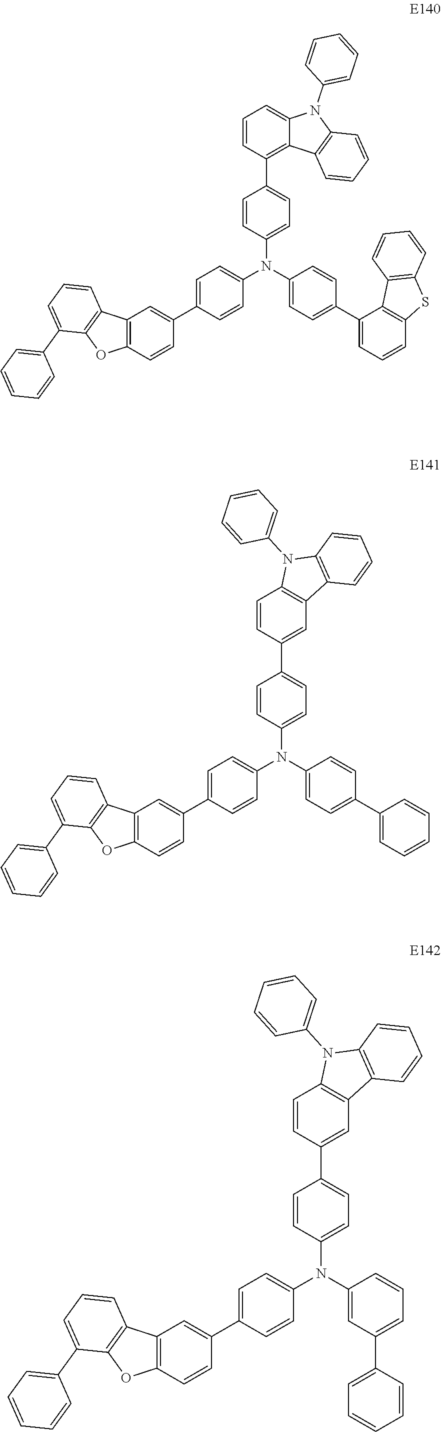 [SMILES: c1ccc(-c2cccc3c2oc2ccc(-c4ccc(N(c5ccc(-c6cccc7sc8ccccc8c67)cc5)c5ccc(-c6cccc7c6c6ccccc6n7-c6ccccc6)cc5)cc4)cc23)cc1 ; c1ccc(-c2ccc(N(c3ccc(-c4ccc5oc6c(-c7ccccc7)cccc6c5c4)cc3)c3ccc(-c4ccc5c(c4)c4ccccc4n5-c4ccccc4)cc3)cc2)cc1 ; c1ccc(-c2cccc(N(c3ccc(-c4ccc5oc6c(-c7ccccc7)cccc6c5c4)cc3)c3ccc(-c4ccc5c(c4)c4ccccc4n5-c4ccccc4)cc3)c2)cc1] 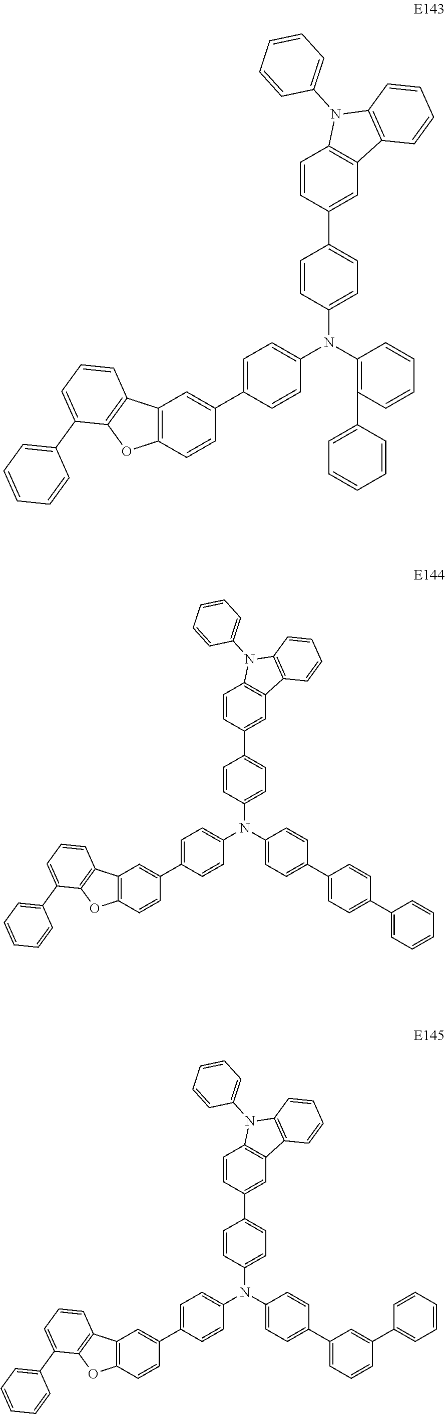 [SMILES: c1ccc(-c2ccccc2N(c2ccc(-c3ccc4oc5c(-c6ccccc6)cccc5c4c3)cc2)c2ccc(-c3ccc4c(c3)c3ccccc3n4-c3ccccc3)cc2)cc1 ; c1ccc(-c2ccc(-c3ccc(N(c4ccc(-c5ccc6oc7c(-c8ccccc8)cccc7c6c5)cc4)c4ccc(-c5ccc6c(c5)c5ccccc5n6-c5ccccc5)cc4)cc3)cc2)cc1 ; c1ccc(-c2cccc(-c3ccc(N(c4ccc(-c5ccc6oc7c(-c8ccccc8)cccc7c6c5)cc4)c4ccc(-c5ccc6c(c5)c5ccccc5n6-c5ccccc5)cc4)cc3)c2)cc1] 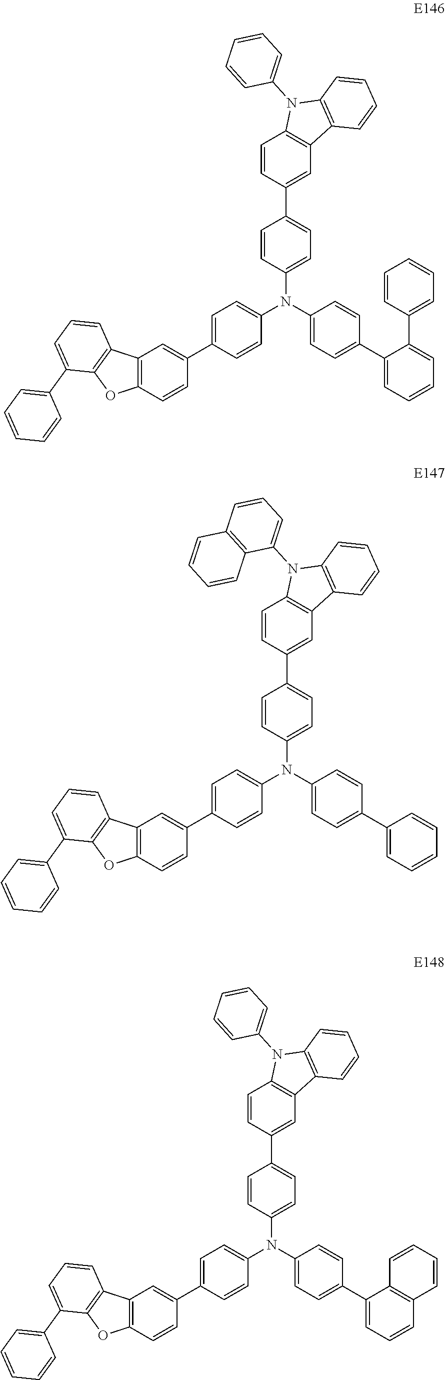 [SMILES: c1ccc(-c2ccccc2-c2ccc(N(c3ccc(-c4ccc5oc6c(-c7ccccc7)cccc6c5c4)cc3)c3ccc(-c4ccc5c(c4)c4ccccc4n5-c4ccccc4)cc3)cc2)cc1 ; c1ccc(-c2ccc(N(c3ccc(-c4ccc5oc6c(-c7ccccc7)cccc6c5c4)cc3)c3ccc(-c4ccc5c(c4)c4ccccc4n5-c4cccc5ccccc45)cc3)cc2)cc1 ; c1ccc(-c2cccc3c2oc2ccc(-c4ccc(N(c5ccc(-c6ccc7c(c6)c6ccccc6n7-c6ccccc6)cc5)c5ccc(-c6cccc7ccccc67)cc5)cc4)cc23)cc1] 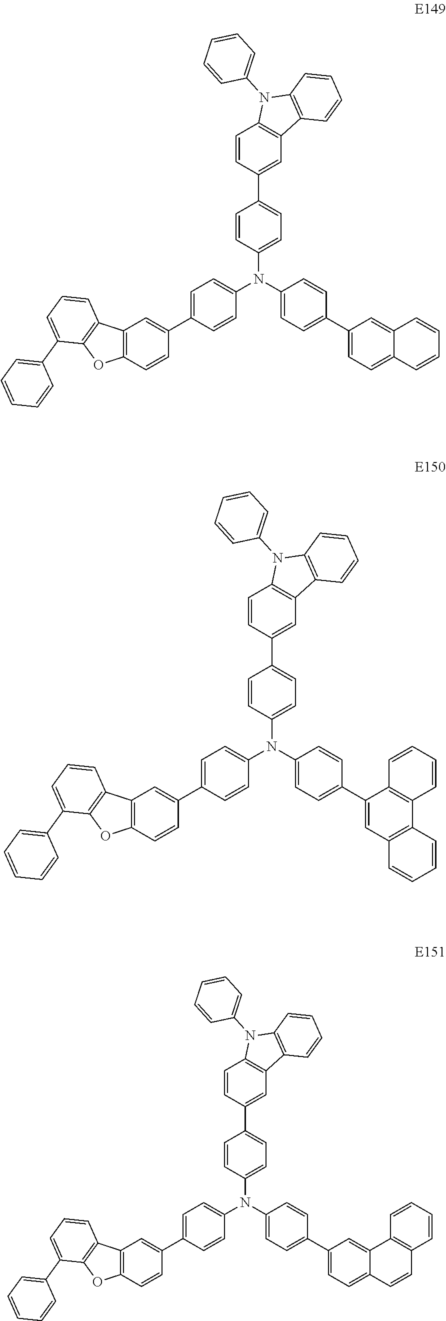 [SMILES: c1ccc(-c2cccc3c2oc2ccc(-c4ccc(N(c5ccc(-c6ccc7ccccc7c6)cc5)c5ccc(-c6ccc7c(c6)c6ccccc6n7-c6ccccc6)cc5)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc(-c6cc7ccccc7c7ccccc67)cc5)cc4)cc23)cc1 ; c1ccc(-c2cccc3c2oc2ccc(-c4ccc(N(c5ccc(-c6ccc7ccc8ccccc8c7c6)cc5)c5ccc(-c6ccc7c(c6)c6ccccc6n7-c6ccccc6)cc5)cc4)cc23)cc1] 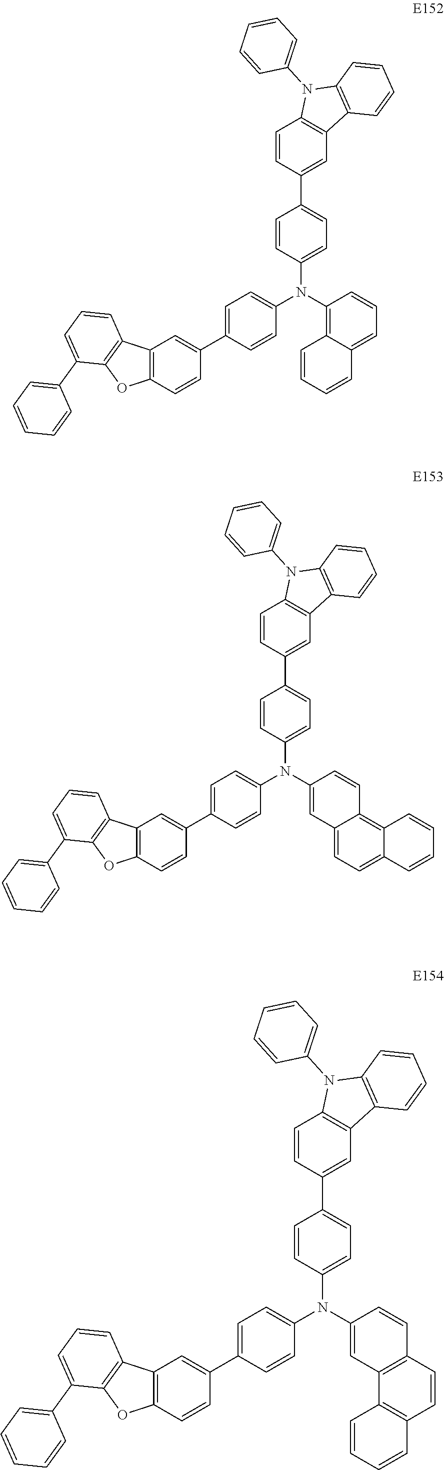 [SMILES: c1ccc(-c2cccc3c2oc2ccc(-c4ccc(N(c5ccc(-c6ccc7c(c6)c6ccccc6n7-c6ccccc6)cc5)c5cccc6ccccc56)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6c(ccc7ccccc76)c5)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6ccc7ccccc7c6c5)cc4)cc23)cc1] 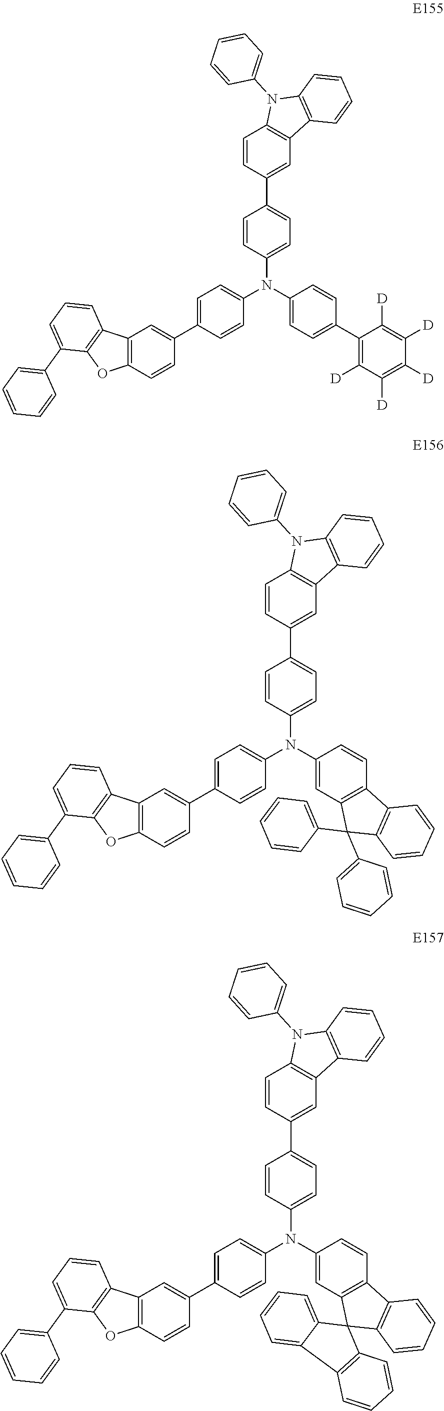 [SMILES: [2H]c1c([2H])c([2H])c(-c2ccc(N(c3ccc(-c4ccc5oc6c(-c7ccccc7)cccc6c5c4)cc3)c3ccc(-c4ccc5c(c4)c4ccccc4n5-c4ccccc4)cc3)cc2)c([2H])c1[2H] ; c1ccc(-c2cccc3c2oc2ccc(-c4ccc(N(c5ccc(-c6ccc7c(c6)c6ccccc6n7-c6ccccc6)cc5)c5ccc6c(c5)C(c5ccccc5)(c5ccccc5)c5ccccc5-6)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6c(c5)C5(c7ccccc7-c7ccccc75)c5ccccc5-6)cc4)cc23)cc1] 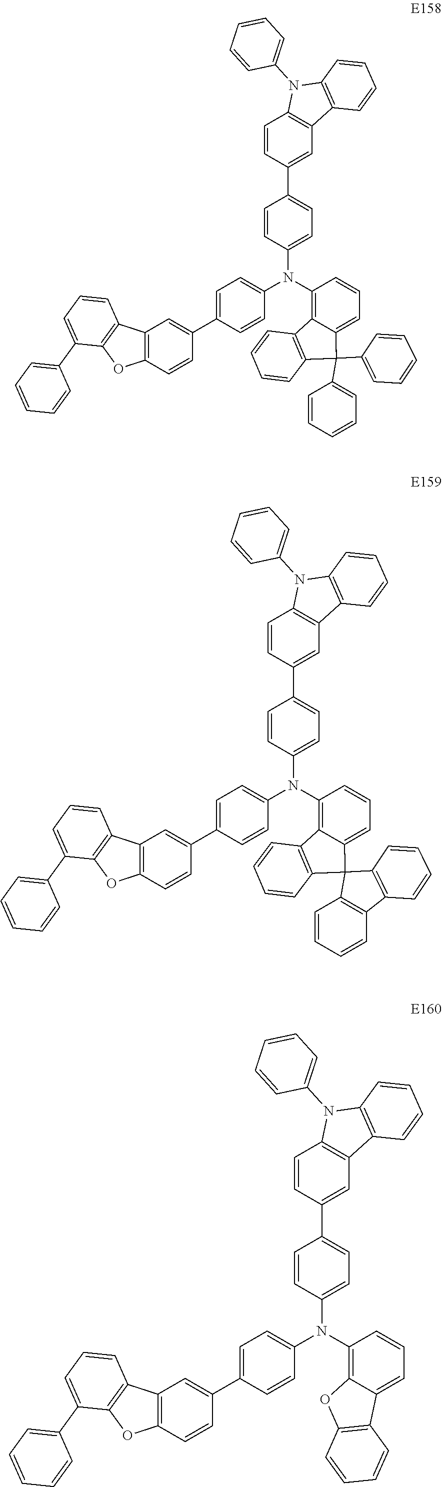 [SMILES: c1ccc(-c2cccc3c2oc2ccc(-c4ccc(N(c5ccc(-c6ccc7c(c6)c6ccccc6n7-c6ccccc6)cc5)c5cccc6c5-c5ccccc5C6(c5ccccc5)c5ccccc5)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6c5-c5ccccc5C65c6ccccc6-c6ccccc65)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6c5oc5ccccc56)cc4)cc23)cc1] 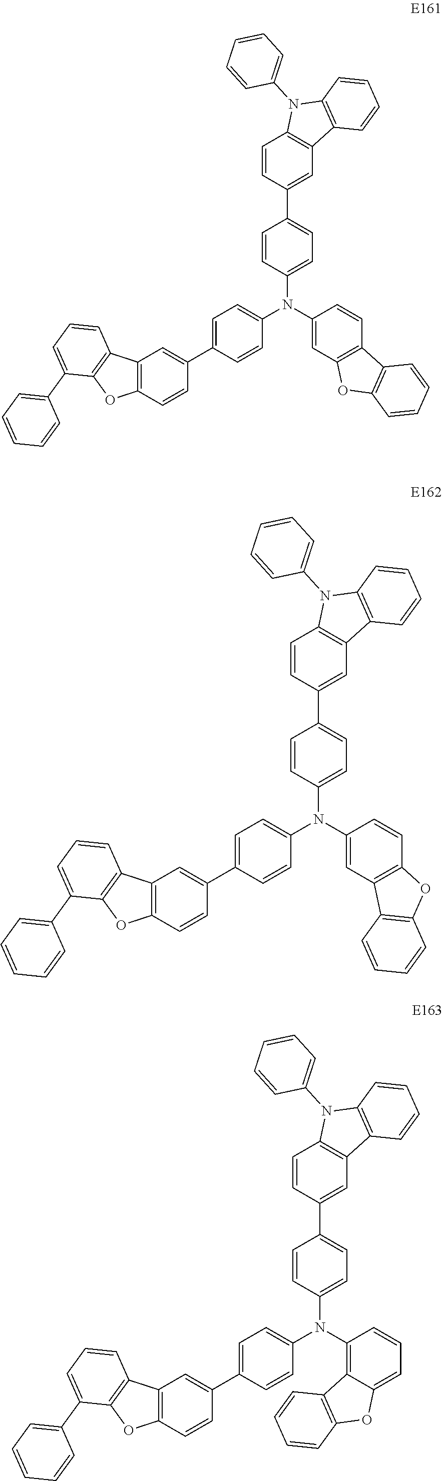 [SMILES: c1ccc(-c2cccc3c2oc2ccc(-c4ccc(N(c5ccc(-c6ccc7c(c6)c6ccccc6n7-c6ccccc6)cc5)c5ccc6c(c5)oc5ccccc56)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6oc7ccccc7c6c5)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6oc7ccccc7c56)cc4)cc23)cc1] 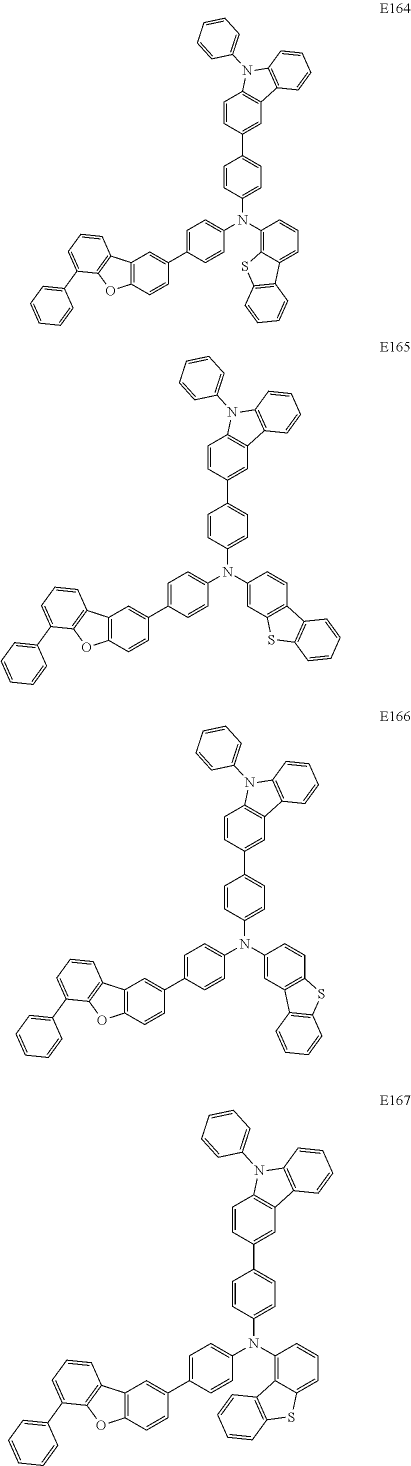 [SMILES: c1ccc(-c2cccc3c2oc2ccc(-c4ccc(N(c5ccc(-c6ccc7c(c6)c6ccccc6n7-c6ccccc6)cc5)c5cccc6c5sc5ccccc56)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6c(c5)sc5ccccc56)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc6sc7ccccc7c6c5)cc4)cc23)cc1 ; c1ccc(-c2cccc3c2oc2ccc(-c4ccc(N(c5ccc(-c6ccc7c(c6)c6ccccc6n7-c6ccccc6)cc5)c5cccc6sc7ccccc7c56)cc4)cc23)cc1] 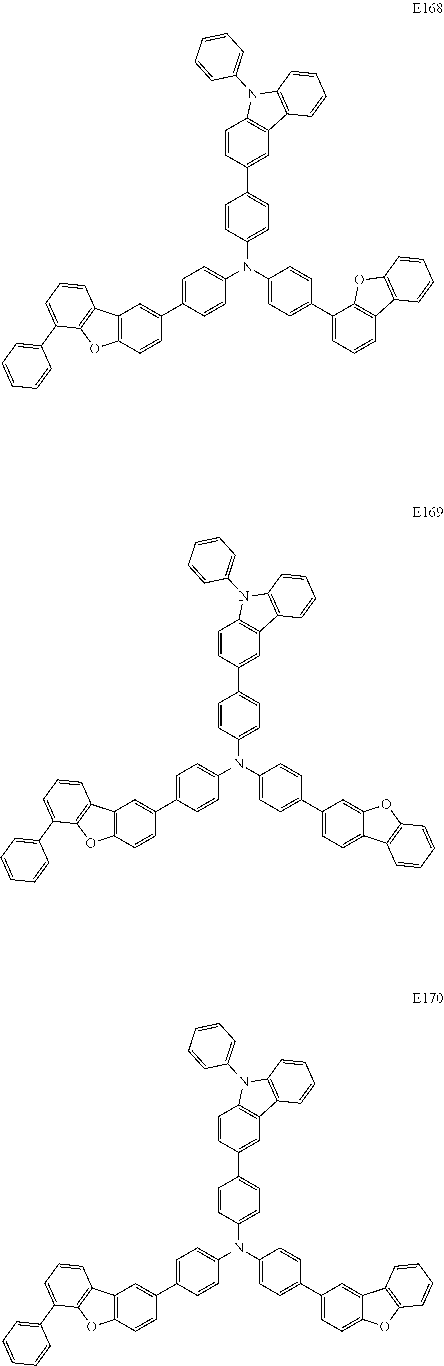 [SMILES: c1ccc(-c2cccc3c2oc2ccc(-c4ccc(N(c5ccc(-c6ccc7c(c6)c6ccccc6n7-c6ccccc6)cc5)c5ccc(-c6cccc7c6oc6ccccc67)cc5)cc4)cc23)cc1 ; c1ccc(-c2cccc3c2oc2ccc(-c4ccc(N(c5ccc(-c6ccc7c(c6)oc6ccccc67)cc5)c5ccc(-c6ccc7c(c6)c6ccccc6n7-c6ccccc6)cc5)cc4)cc23)cc1 ; c1ccc(-c2cccc3c2oc2ccc(-c4ccc(N(c5ccc(-c6ccc7oc8ccccc8c7c6)cc5)c5ccc(-c6ccc7c(c6)c6ccccc6n7-c6ccccc6)cc5)cc4)cc23)cc1] 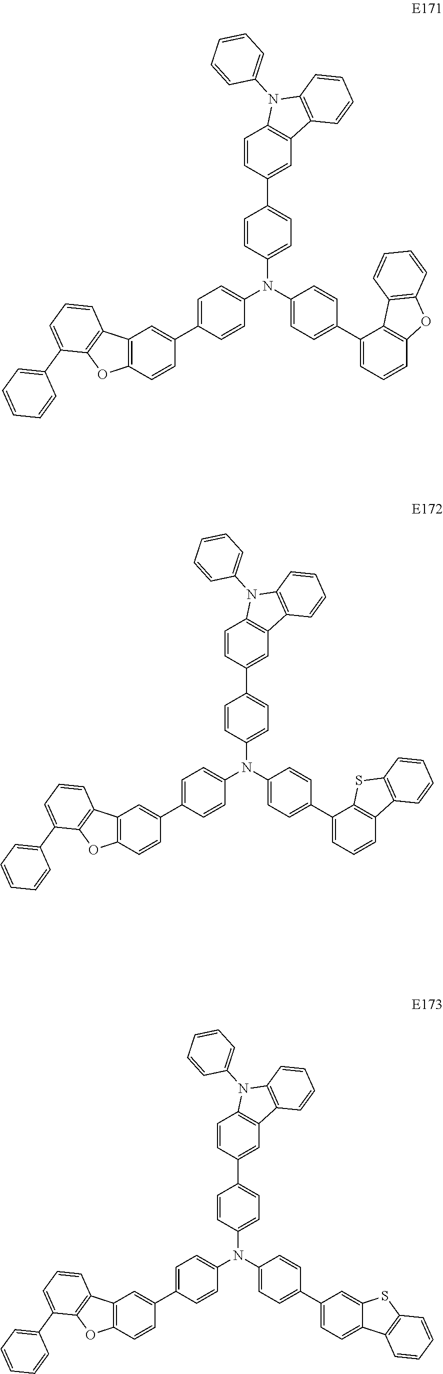 [SMILES: c1ccc(-c2cccc3c2oc2ccc(-c4ccc(N(c5ccc(-c6ccc7c(c6)c6ccccc6n7-c6ccccc6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6ccc7c(c6)c6ccccc6n7-c6ccccc6)cc5)cc4)cc23)cc1] 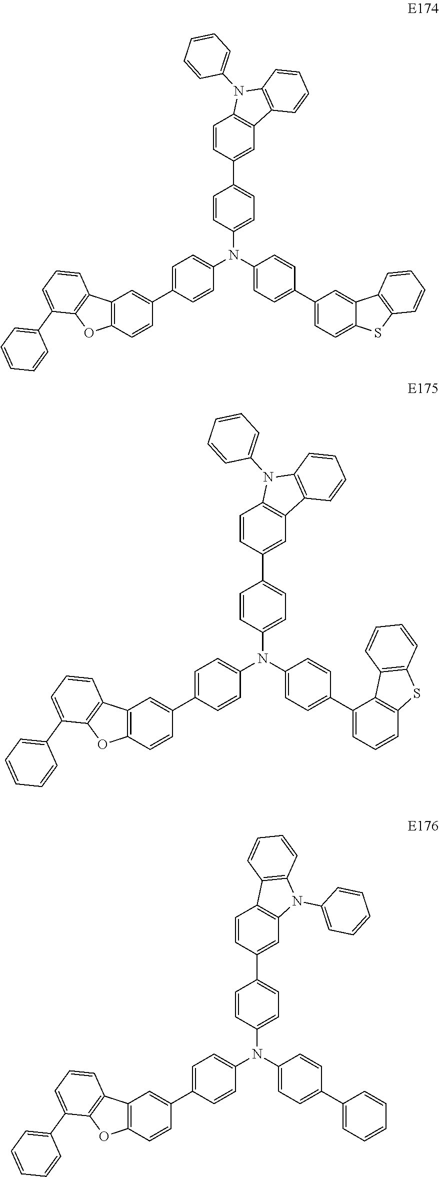 [SMILES: c1ccc(-c2cccc3c2oc2ccc(-c4ccc(N(c5ccc(-c6ccc7sc8ccccc8c7c6)cc5)c5ccc(-c6ccc7c(c6)c6ccccc6n7-c6ccccc6)cc5)cc4)cc23)cc1 ; c1ccc(-c2cccc3c2oc2ccc(-c4ccc(N(c5ccc(-c6ccc7c(c6)c6ccccc6n7-c6ccccc6)cc5)c5ccc(-c6cccc7sc8ccccc8c67)cc5)cc4)cc23)cc1 ; c1ccc(-c2ccc(N(c3ccc(-c4ccc5oc6c(-c7ccccc7)cccc6c5c4)cc3)c3ccc(-c4ccc5c6ccccc6n(-c6ccccc6)c5c4)cc3)cc2)cc1] 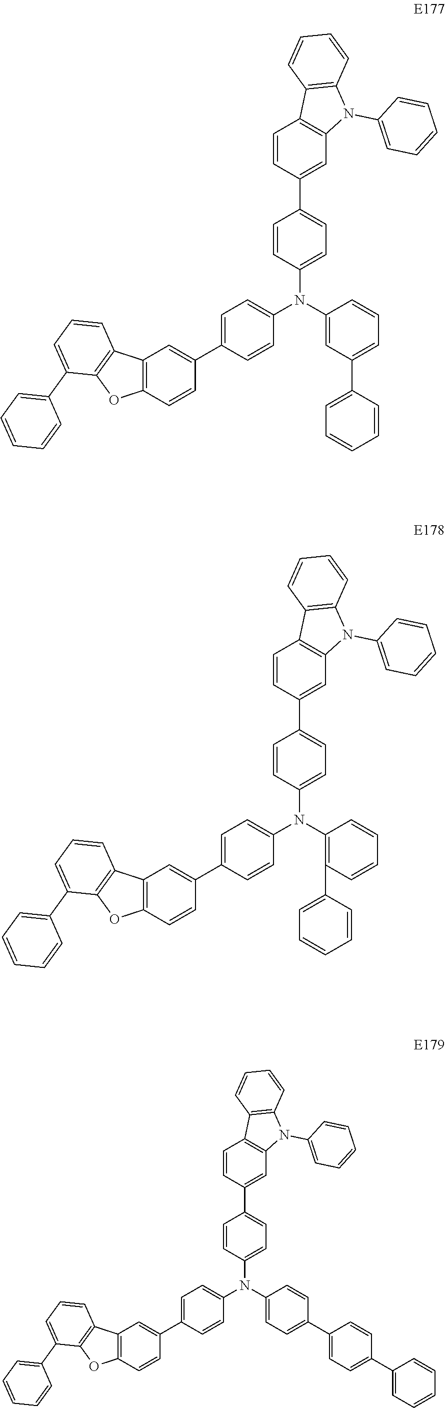 [SMILES: c1ccc(-c2cccc(N(c3ccc(-c4ccc5oc6c(-c7ccccc7)cccc6c5c4)cc3)c3ccc(-c4ccc5c6ccccc6n(-c6ccccc6)c5c4)cc3)c2)cc1 ; c1ccc(-c2ccccc2N(c2ccc(-c3ccc4oc5c(-c6ccccc6)cccc5c4c3)cc2)c2ccc(-c3ccc4c5ccccc5n(-c5ccccc5)c4c3)cc2)cc1 ; c1ccc(-c2ccc(-c3ccc(N(c4ccc(-c5ccc6oc7c(-c8ccccc8)cccc7c6c5)cc4)c4ccc(-c5ccc6c7ccccc7n(-c7ccccc7)c6c5)cc4)cc3)cc2)cc1] 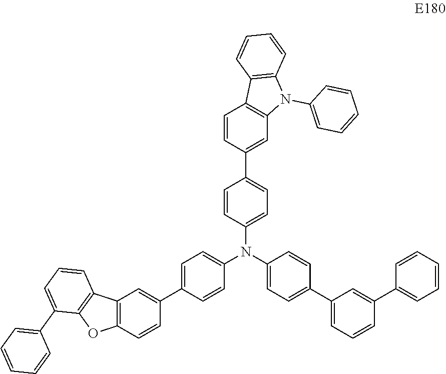 [SMILES: c1ccc(-c2cccc(-c3ccc(N(c4ccc(-c5ccc6oc7c(-c8ccccc8)cccc7c6c5)cc4)c4ccc(-c5ccc6c7ccccc7n(-c7ccccc7)c6c5)cc4)cc3)c2)cc1] 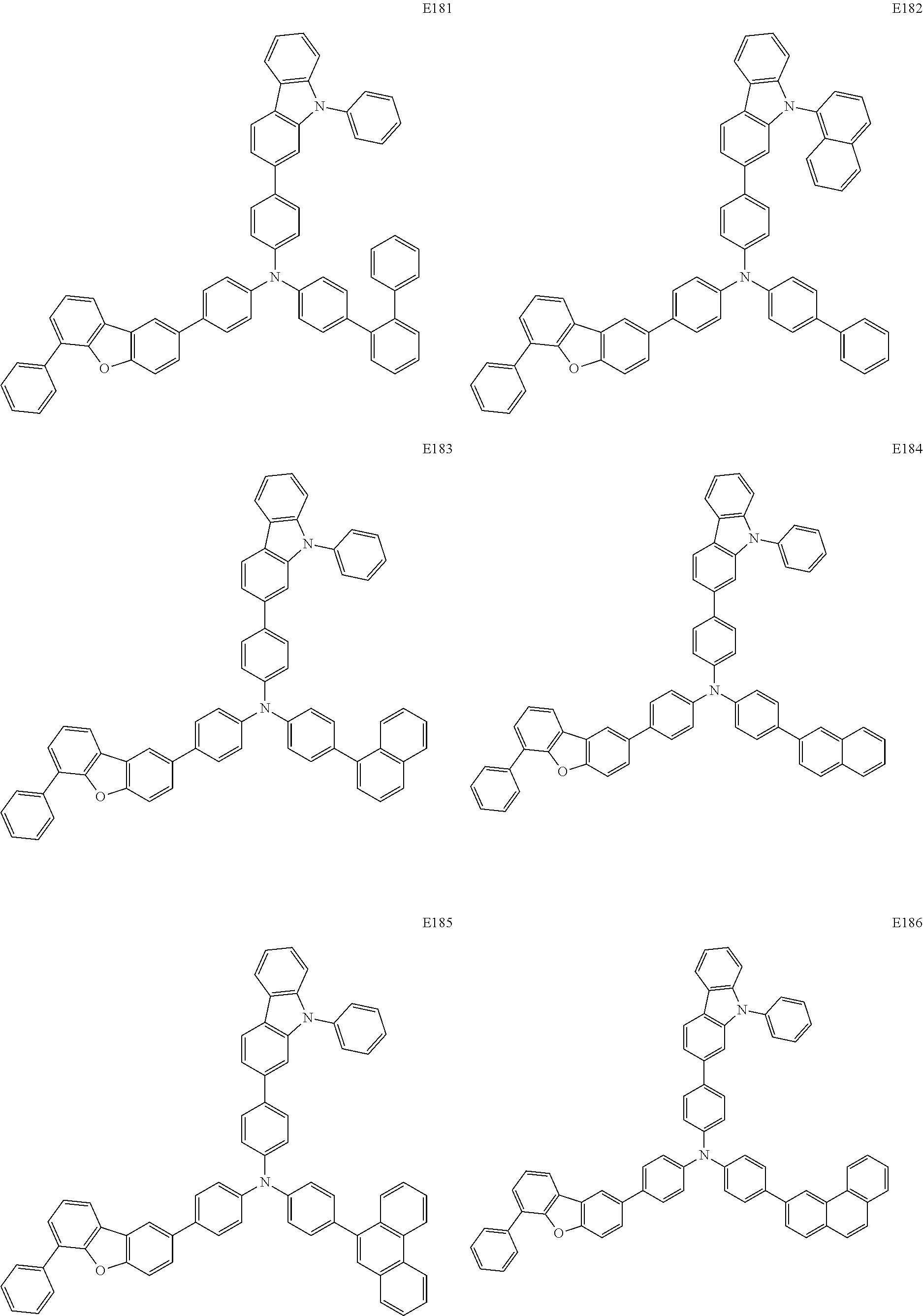 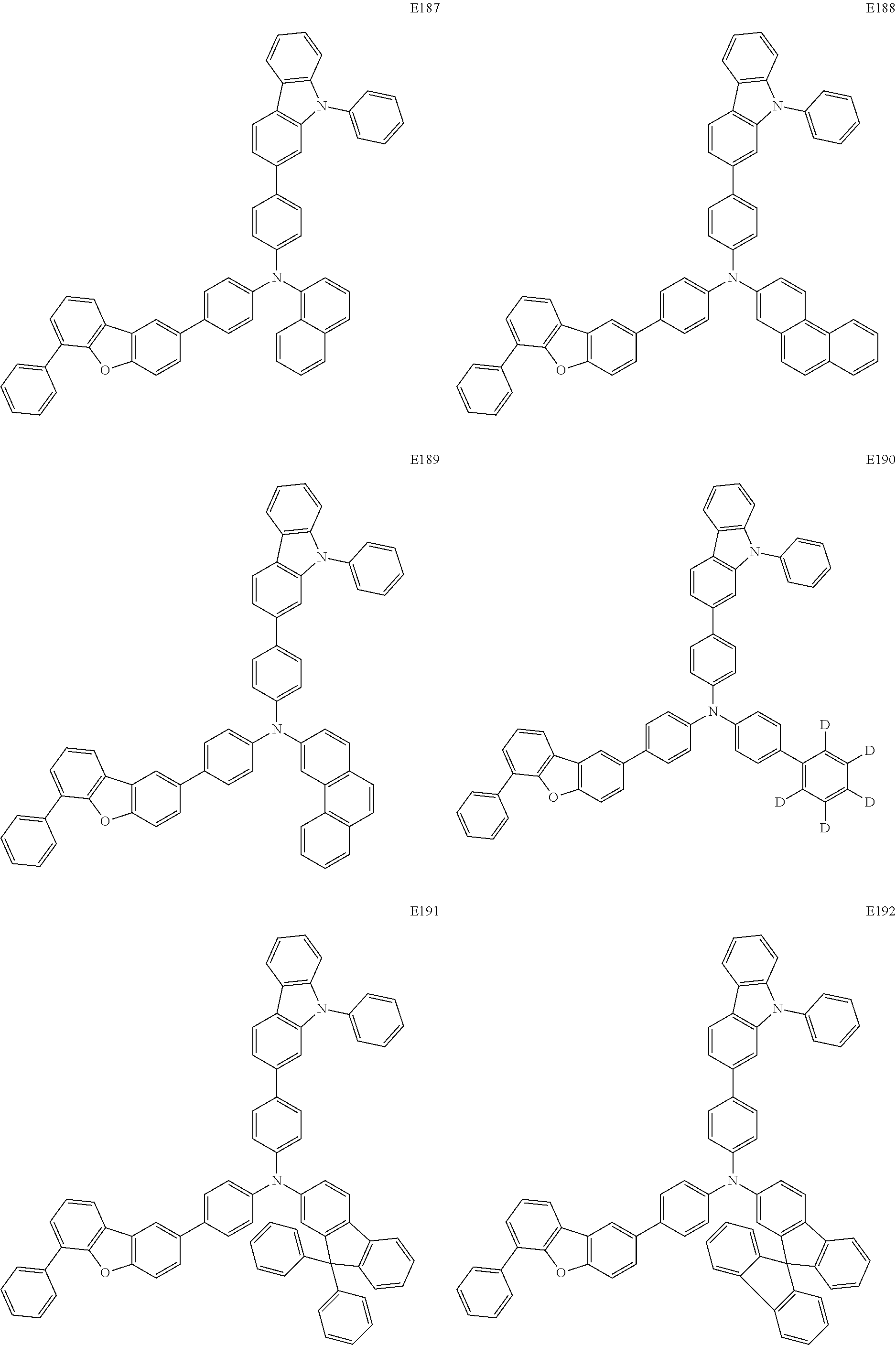 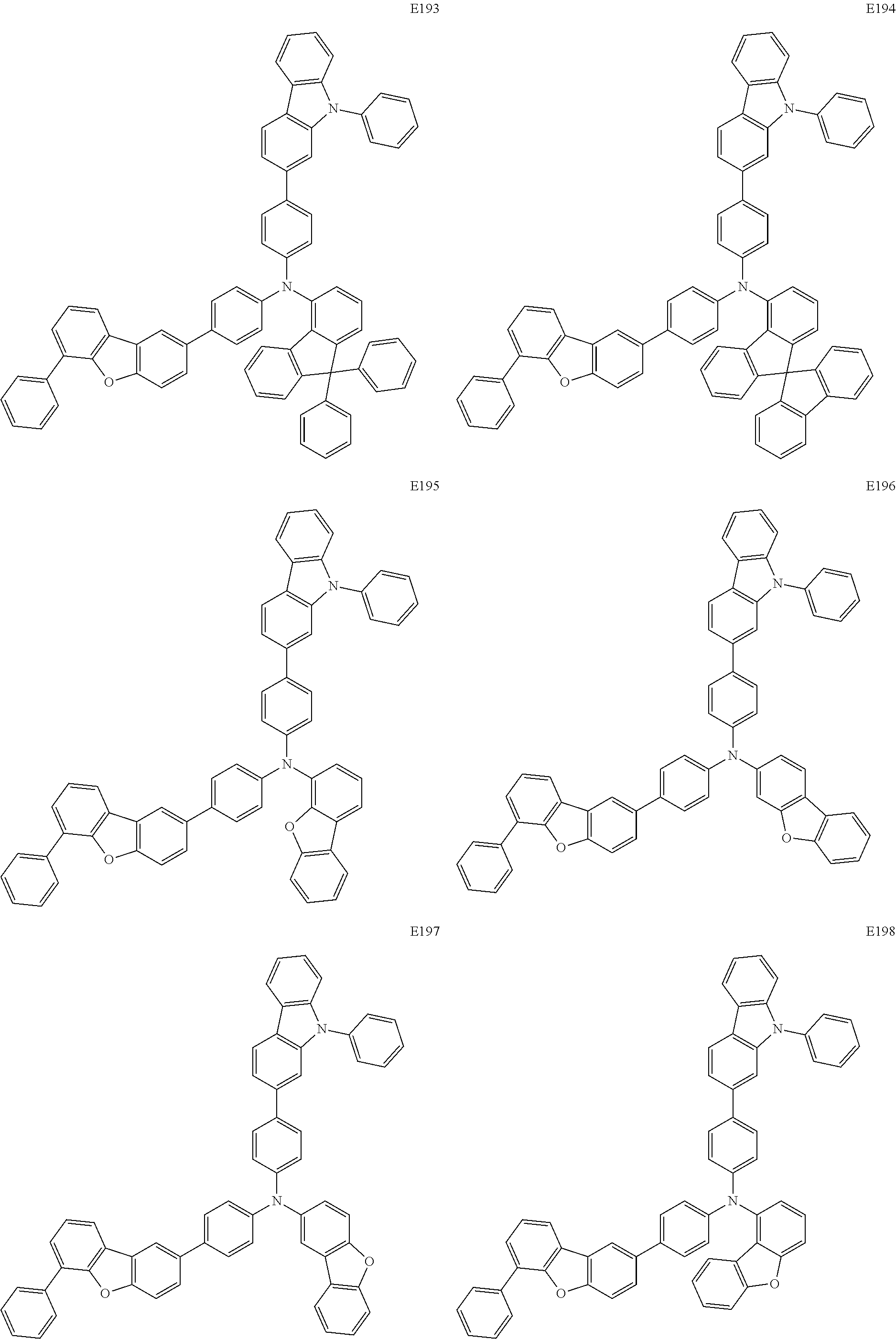 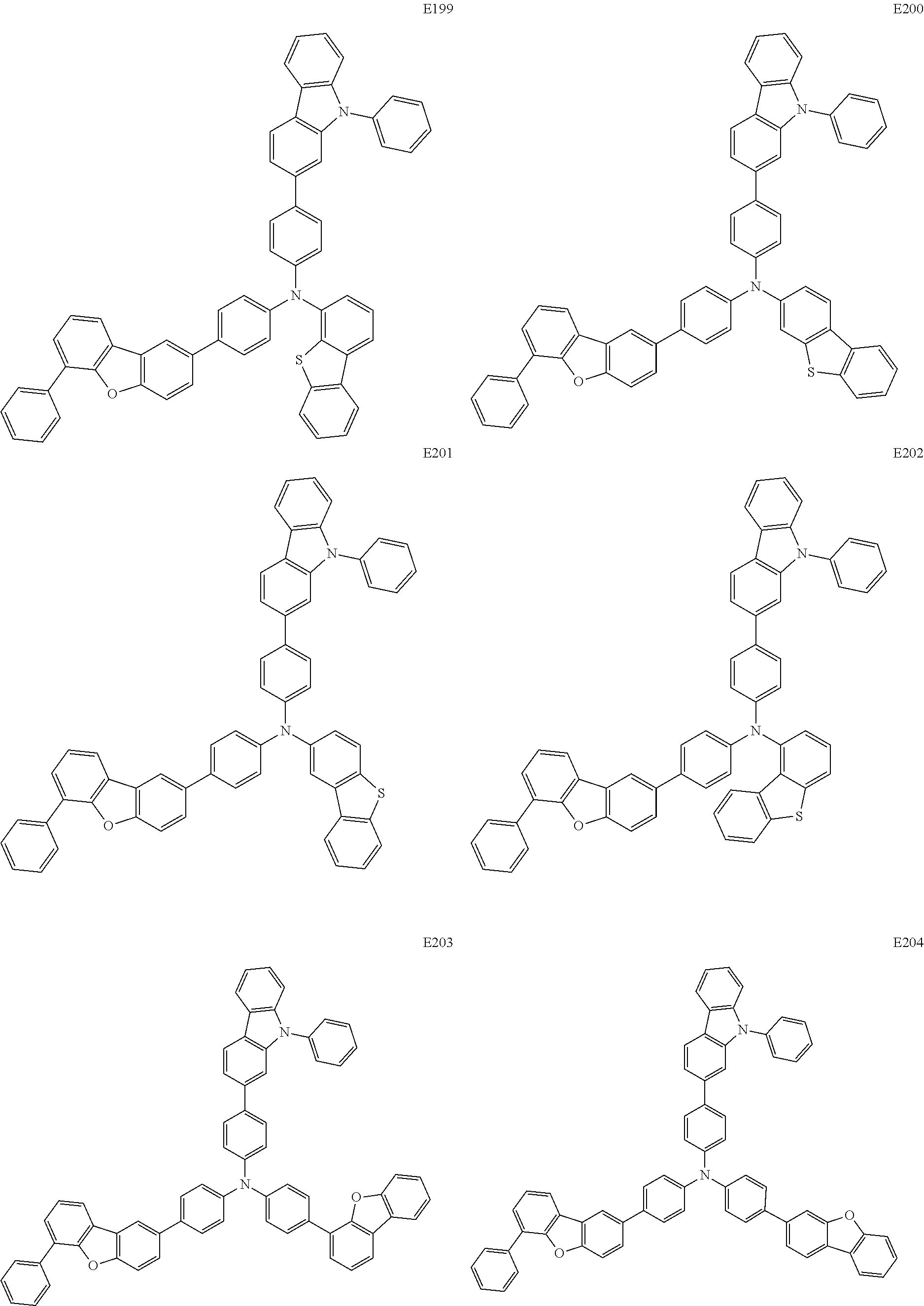 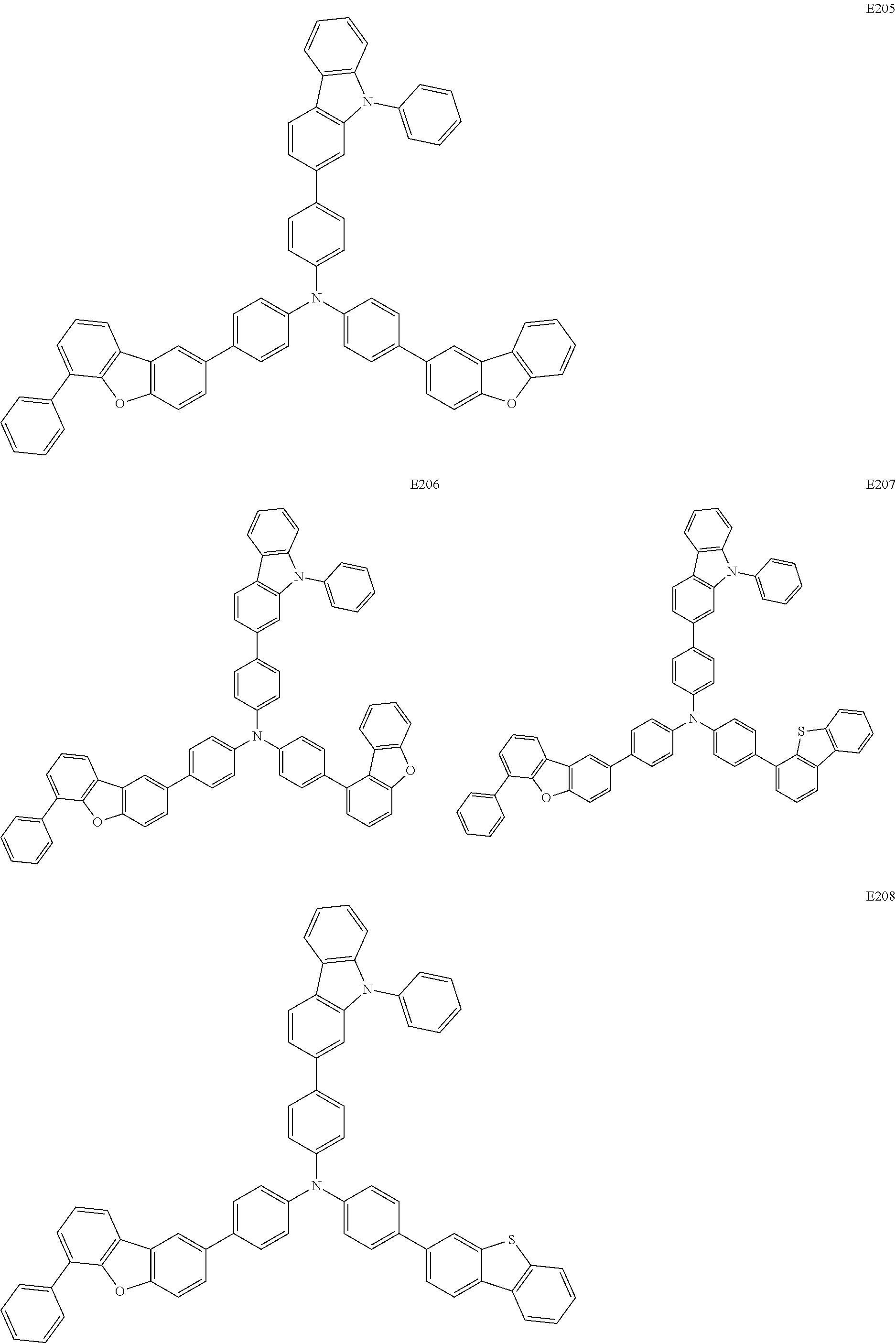 [SMILES: c1ccc(-c2cccc3c2oc2ccc(-c4ccc(N(c5ccc(-c6ccc7oc8ccccc8c7c6)cc5)c5ccc(-c6ccc7c8ccccc8n(-c8ccccc8)c7c6)cc5)cc4)cc23)cc1 ; c1ccc(-c2cccc3c2oc2ccc(-c4ccc(N(c5ccc(-c6ccc7c8ccccc8n(-c8ccccc8)c7c6)cc5)c5ccc(-c6cccc7oc8ccccc8c67)cc5)cc4)cc23)cc1 ; c1ccc(-c2cccc3c2oc2ccc(-c4ccc(N(c5ccc(-c6ccc7c8ccccc8n(-c8ccccc8)c7c6)cc5)c5ccc(-c6cccc7c6sc6ccccc67)cc5)cc4)cc23)cc1 ; c1ccc(-c2cccc3c2oc2ccc(-c4ccc(N(c5ccc(-c6ccc7c(c6)sc6ccccc67)cc5)c5ccc(-c6ccc7c8ccccc8n(-c8ccccc8)c7c6)cc5)cc4)cc23)cc1] 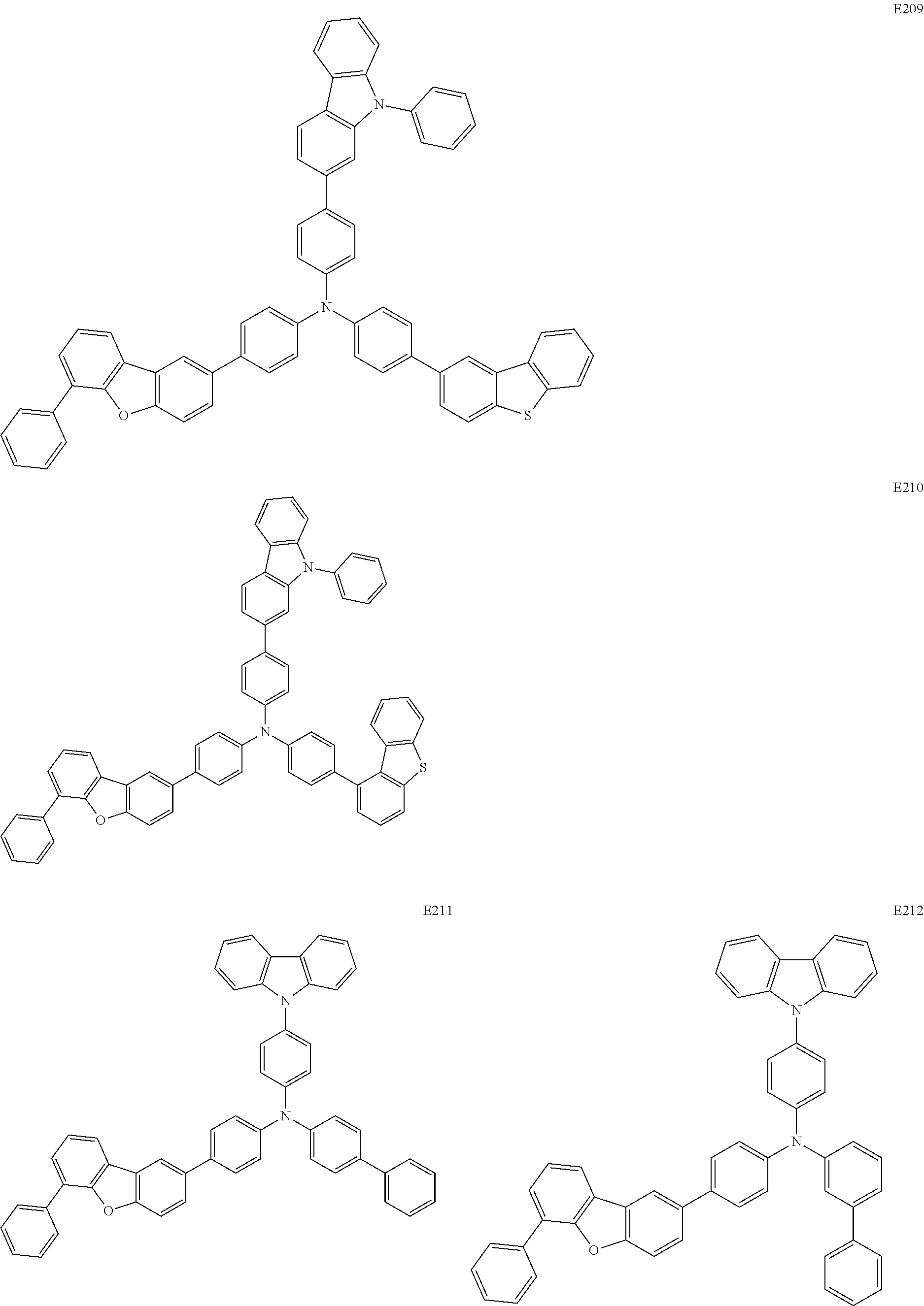 [SMILES: c1ccc(-c2cccc3c2oc2ccc(-c4ccc(N(c5ccc(-c6ccc7sc8ccccc8c7c6)cc5)c5ccc(-c6ccc7c8ccccc8n(-c8ccccc8)c7c6)cc5)cc4)cc23)cc1 ; c1ccc(-c2cccc3c2oc2ccc(-c4ccc(N(c5ccc(-c6ccc7c8ccccc8n(-c8ccccc8)c7c6)cc5)c5ccc(-c6cccc7sc8ccccc8c67)cc5)cc4)cc23)cc1 ; c1ccc(-c2ccc(N(c3ccc(-c4ccc5oc6c(-c7ccccc7)cccc6c5c4)cc3)c3ccc(-n4c5ccccc5c5ccccc54)cc3)cc2)cc1 ; c1ccc(-c2cccc(N(c3ccc(-c4ccc5oc6c(-c7ccccc7)cccc6c5c4)cc3)c3ccc(-n4c5ccccc5c5ccccc54)cc3)c2)cc1] 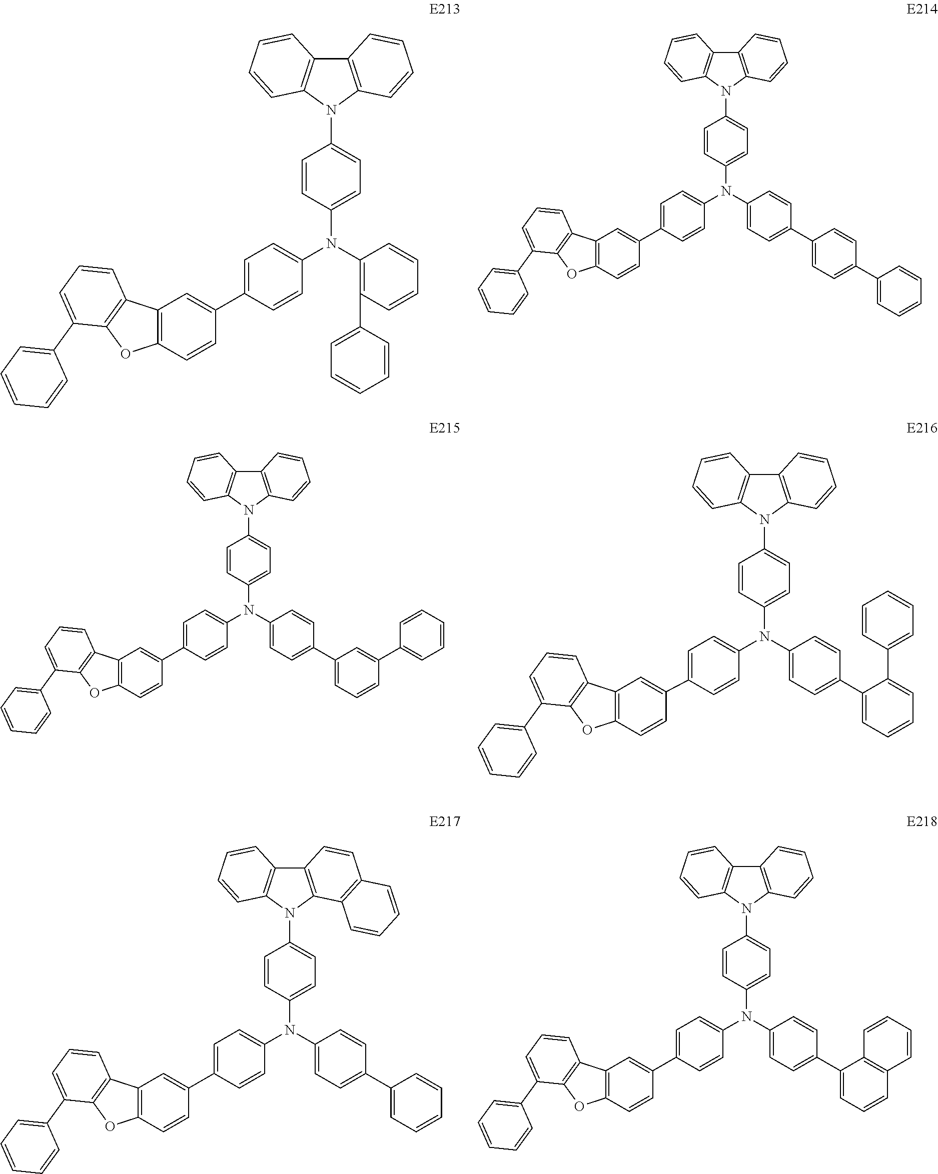 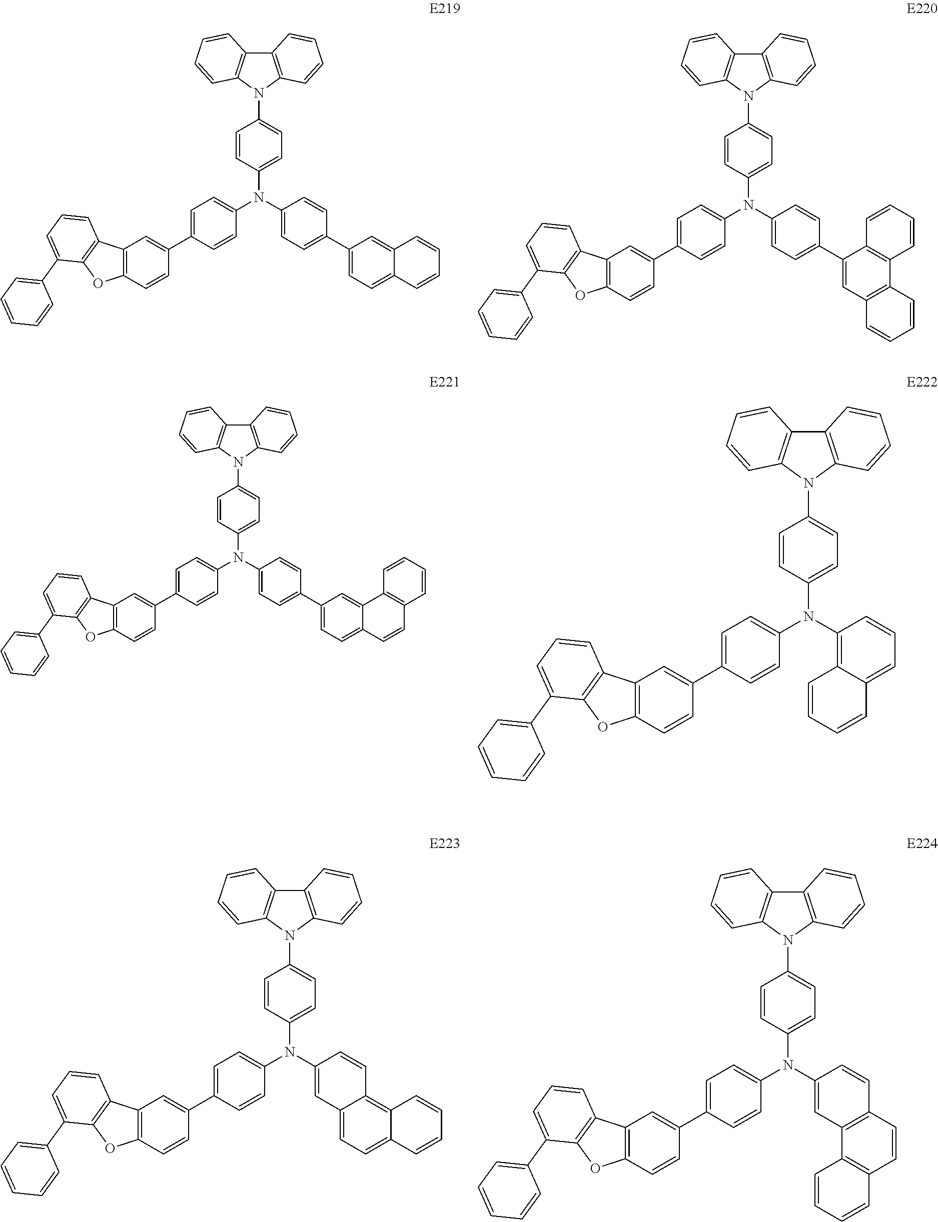 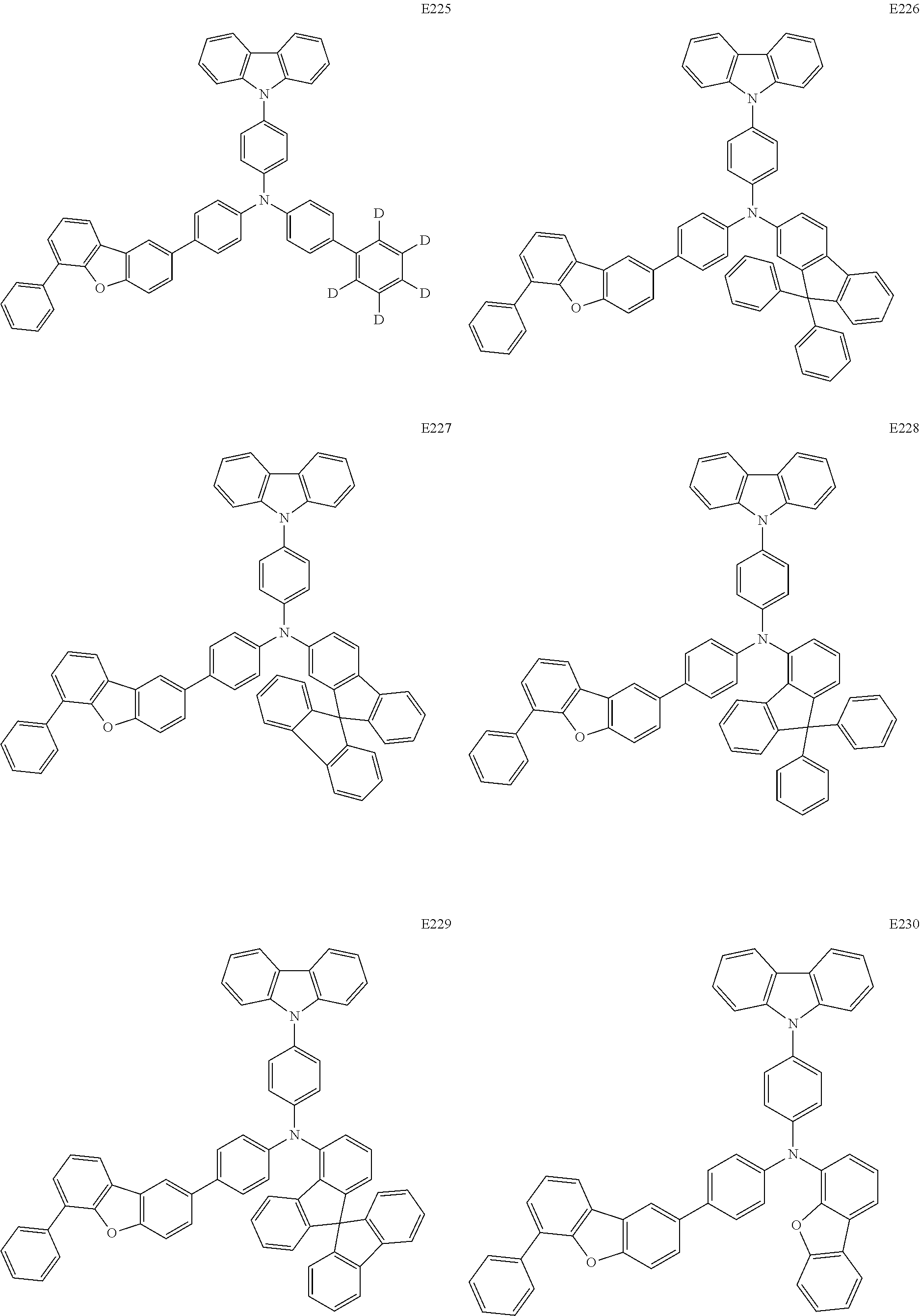 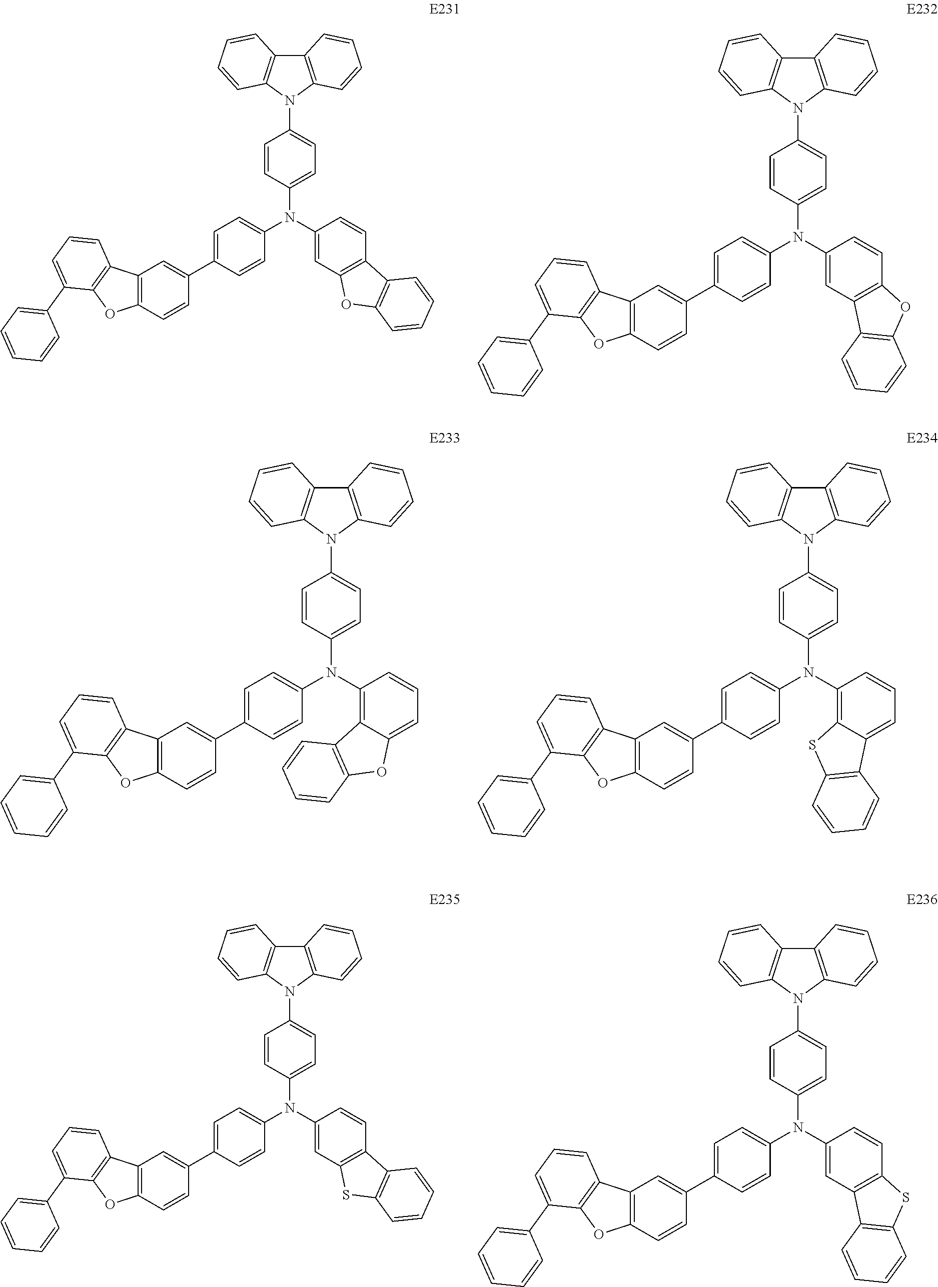 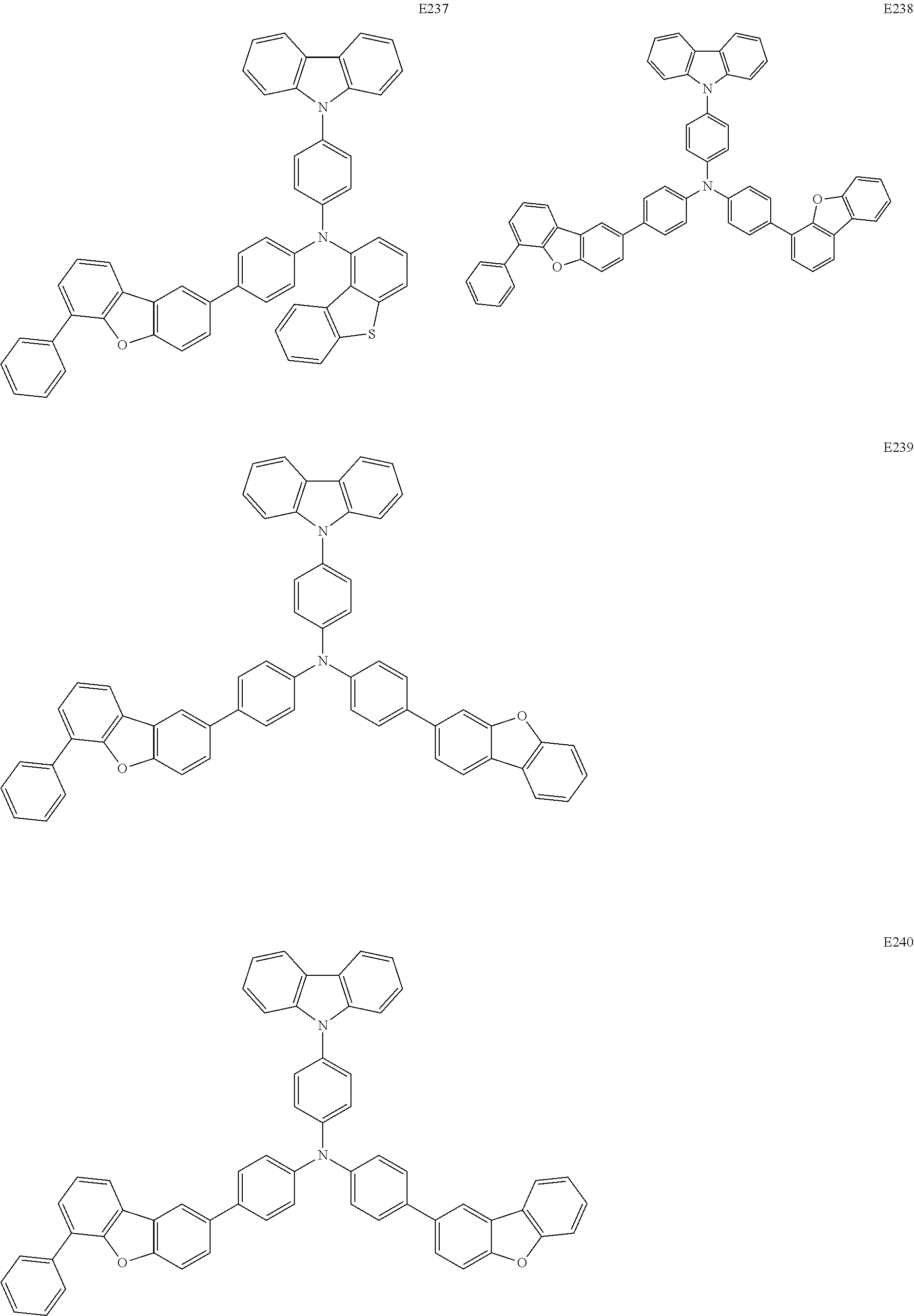 [SMILES: c1ccc(-c2cccc3c2oc2ccc(-c4ccc(N(c5ccc(-n6c7ccccc7c7ccccc76)cc5)c5cccc6sc7ccccc7c56)cc4)cc23)cc1 ; c1ccc(-c2cccc3c2oc2ccc(-c4ccc(N(c5ccc(-c6cccc7c6oc6ccccc67)cc5)c5ccc(-n6c7ccccc7c7ccccc76)cc5)cc4)cc23)cc1 ; c1ccc(-c2cccc3c2oc2ccc(-c4ccc(N(c5ccc(-c6ccc7c(c6)oc6ccccc67)cc5)c5ccc(-n6c7ccccc7c7ccccc76)cc5)cc4)cc23)cc1 ; c1ccc(-c2cccc3c2oc2ccc(-c4ccc(N(c5ccc(-c6ccc7oc8ccccc8c7c6)cc5)c5ccc(-n6c7ccccc7c7ccccc76)cc5)cc4)cc23)cc1] 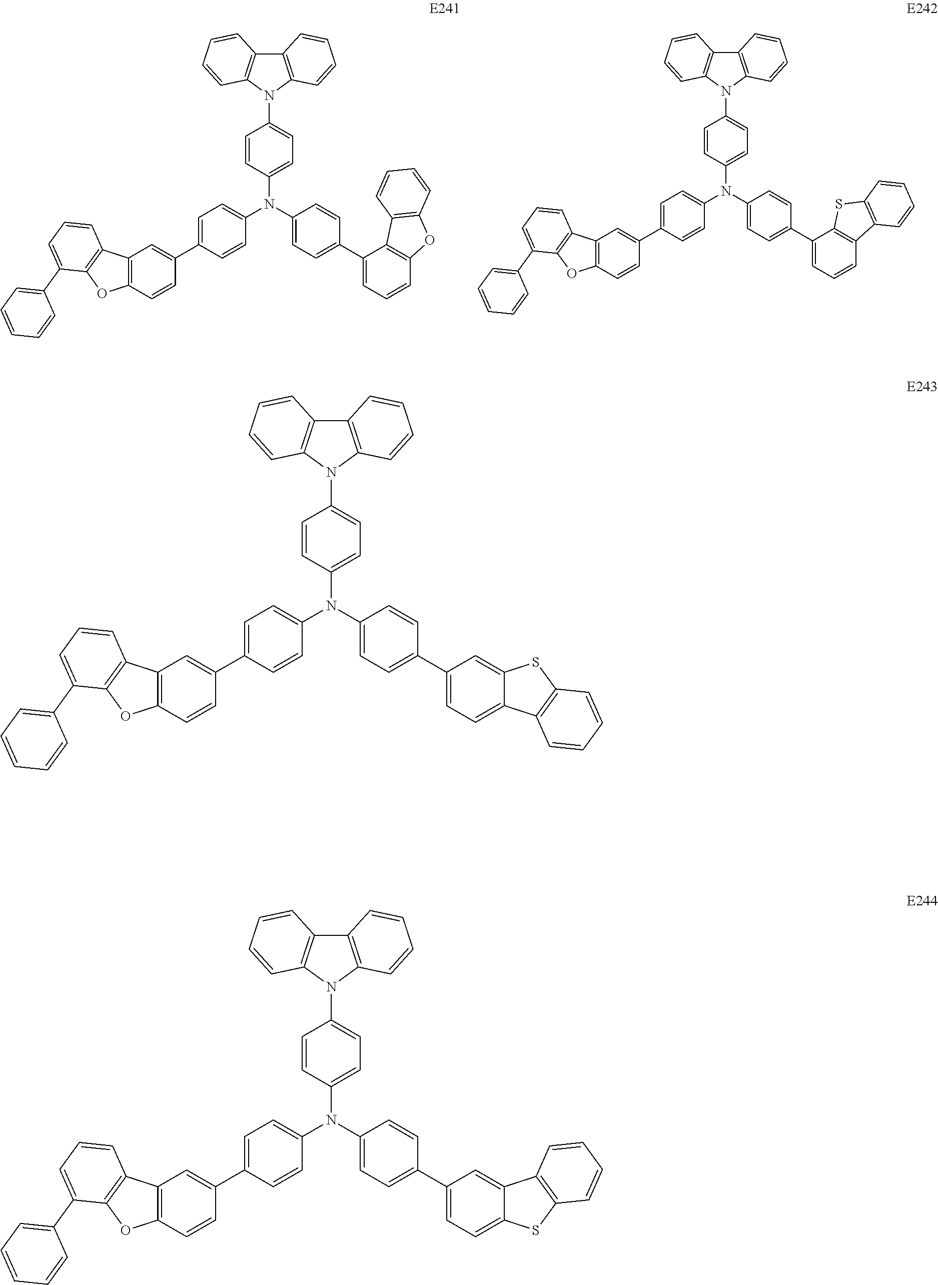 [SMILES: c1ccc(-c2cccc3c2oc2ccc(-c4ccc(N(c5ccc(-c6cccc7oc8ccccc8c67)cc5)c5ccc(-n6c7ccccc7c7ccccc76)cc5)cc4)cc23)cc1 ; c1ccc(-c2cccc3c2oc2ccc(-c4ccc(N(c5ccc(-c6cccc7c6sc6ccccc67)cc5)c5ccc(-n6c7ccccc7c7ccccc76)cc5)cc4)cc23)cc1 ; c1ccc(-c2cccc3c2oc2ccc(-c4ccc(N(c5ccc(-c6ccc7c(c6)sc6ccccc67)cc5)c5ccc(-n6c7ccccc7c7ccccc76)cc5)cc4)cc23)cc1 ; c1ccc(-c2cccc3c2oc2ccc(-c4ccc(N(c5ccc(-c6ccc7sc8ccccc8c7c6)cc5)c5ccc(-n6c7ccccc7c7ccccc76)cc5)cc4)cc23)cc1] 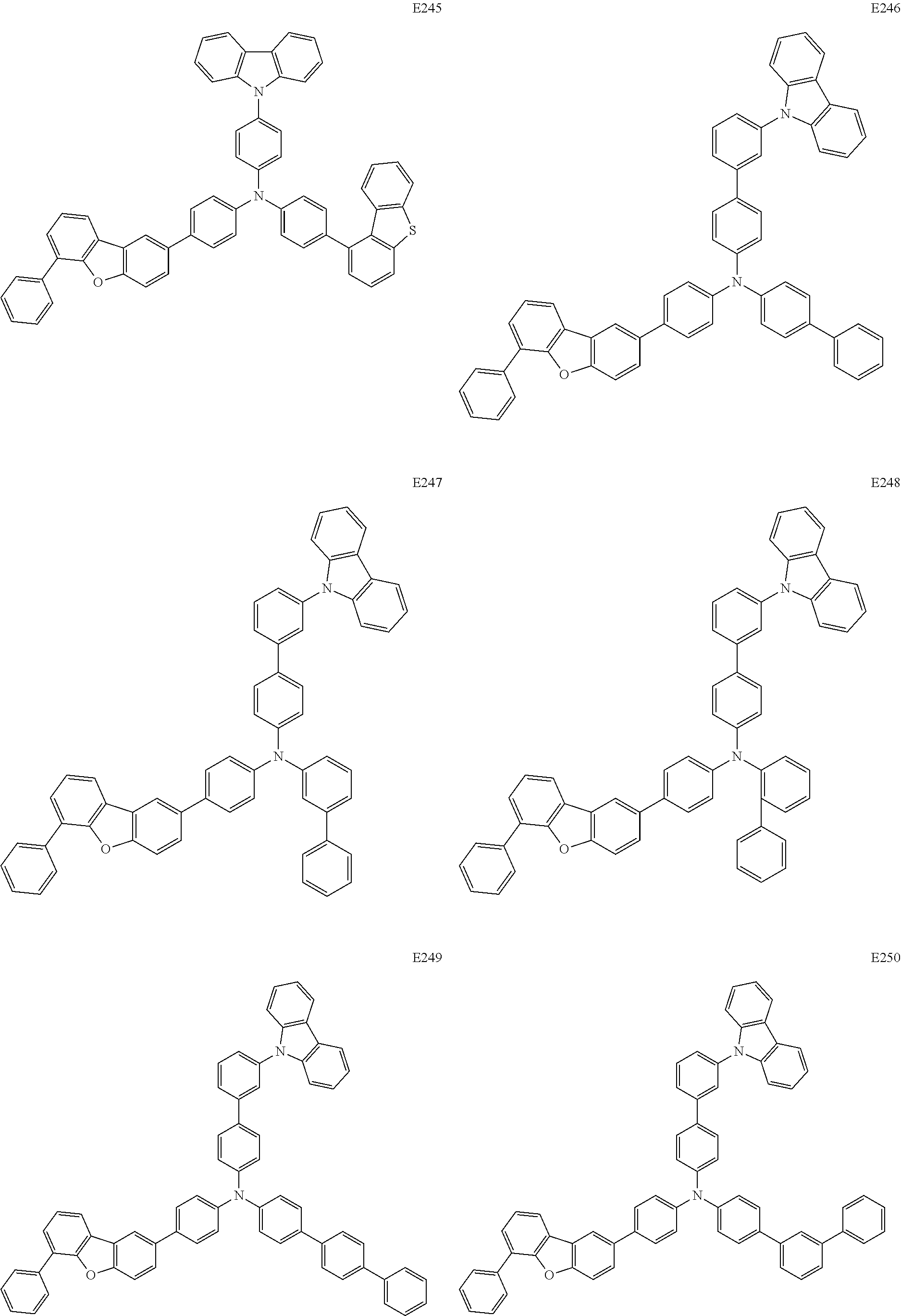 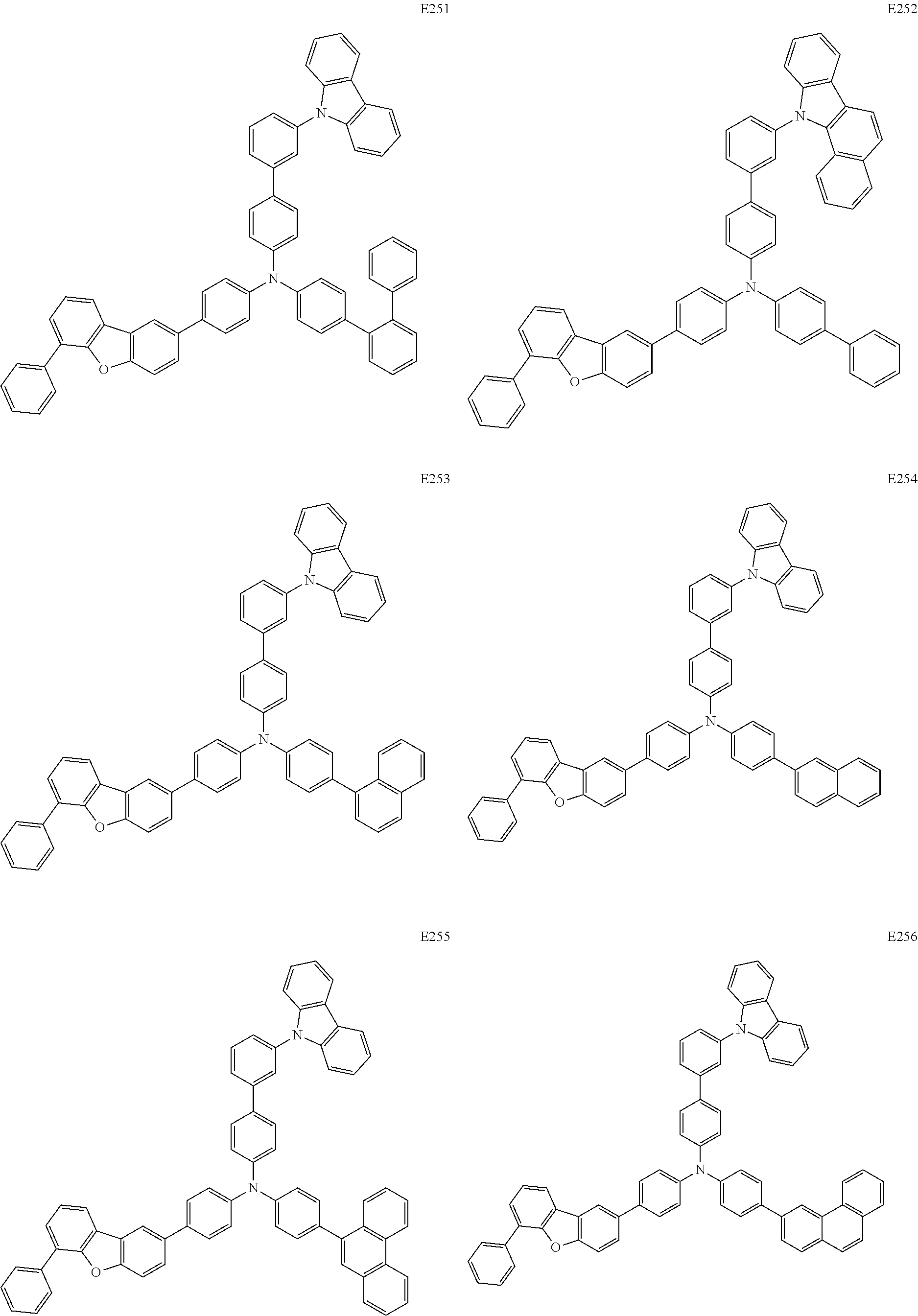 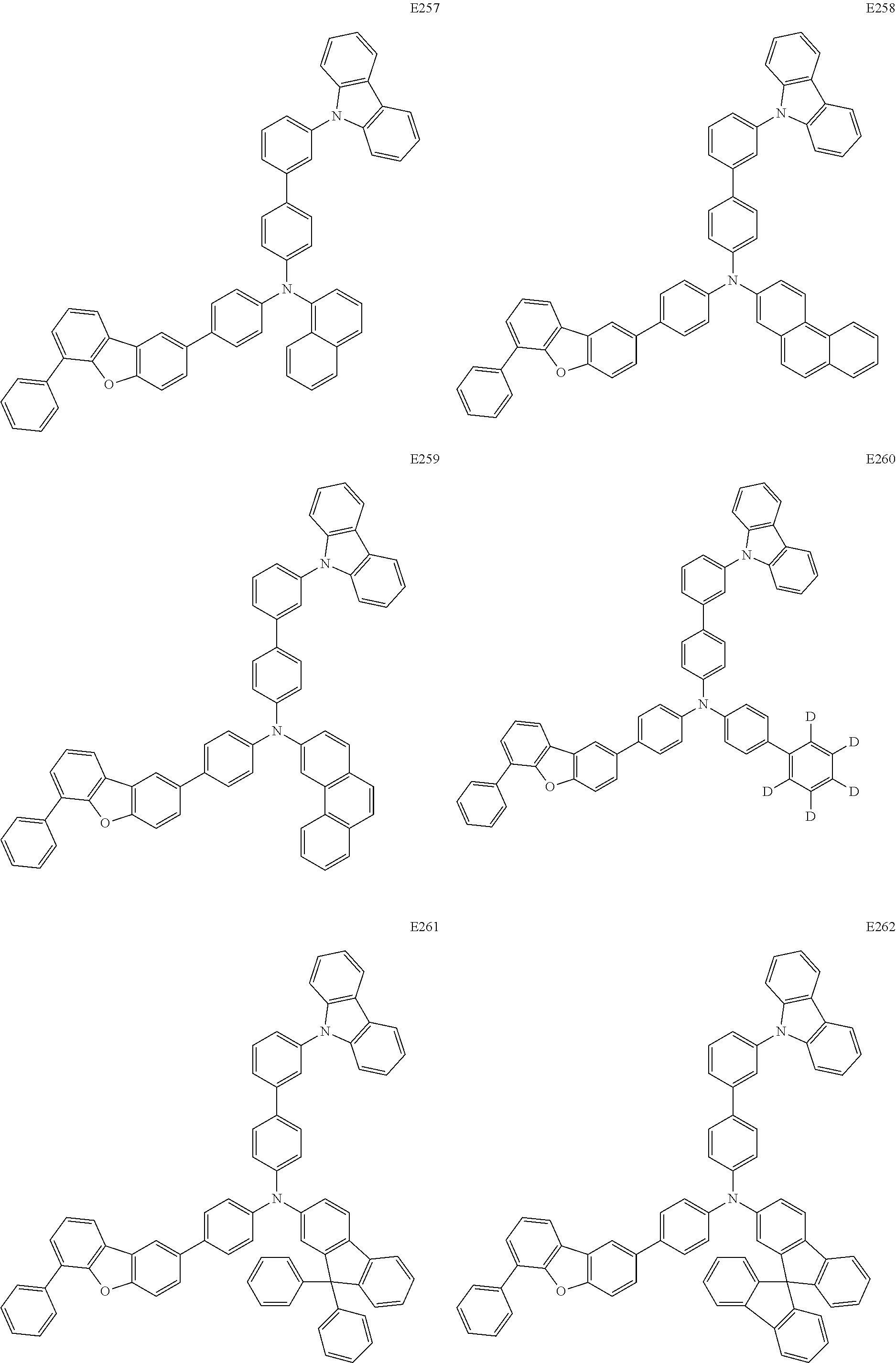 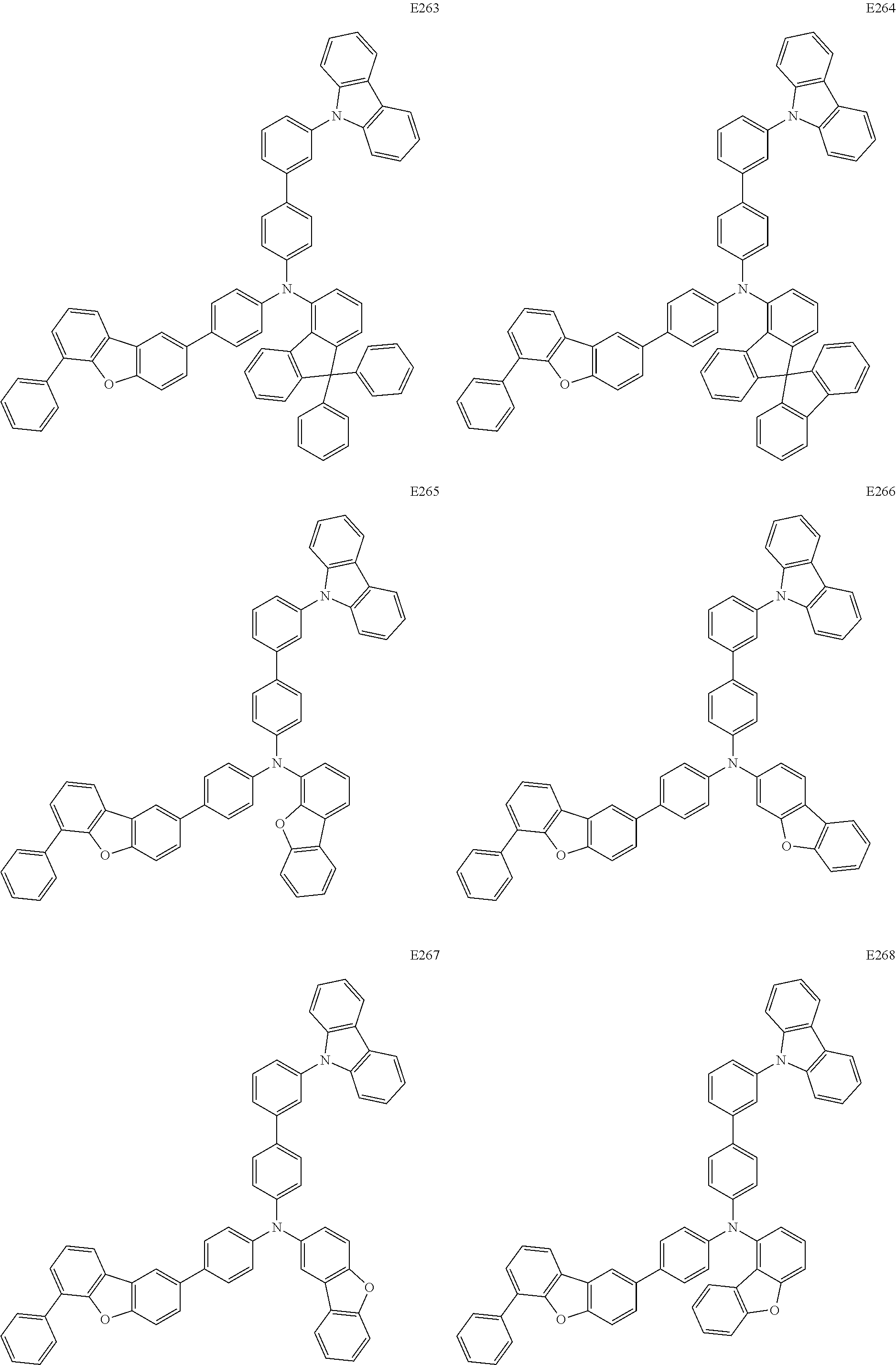 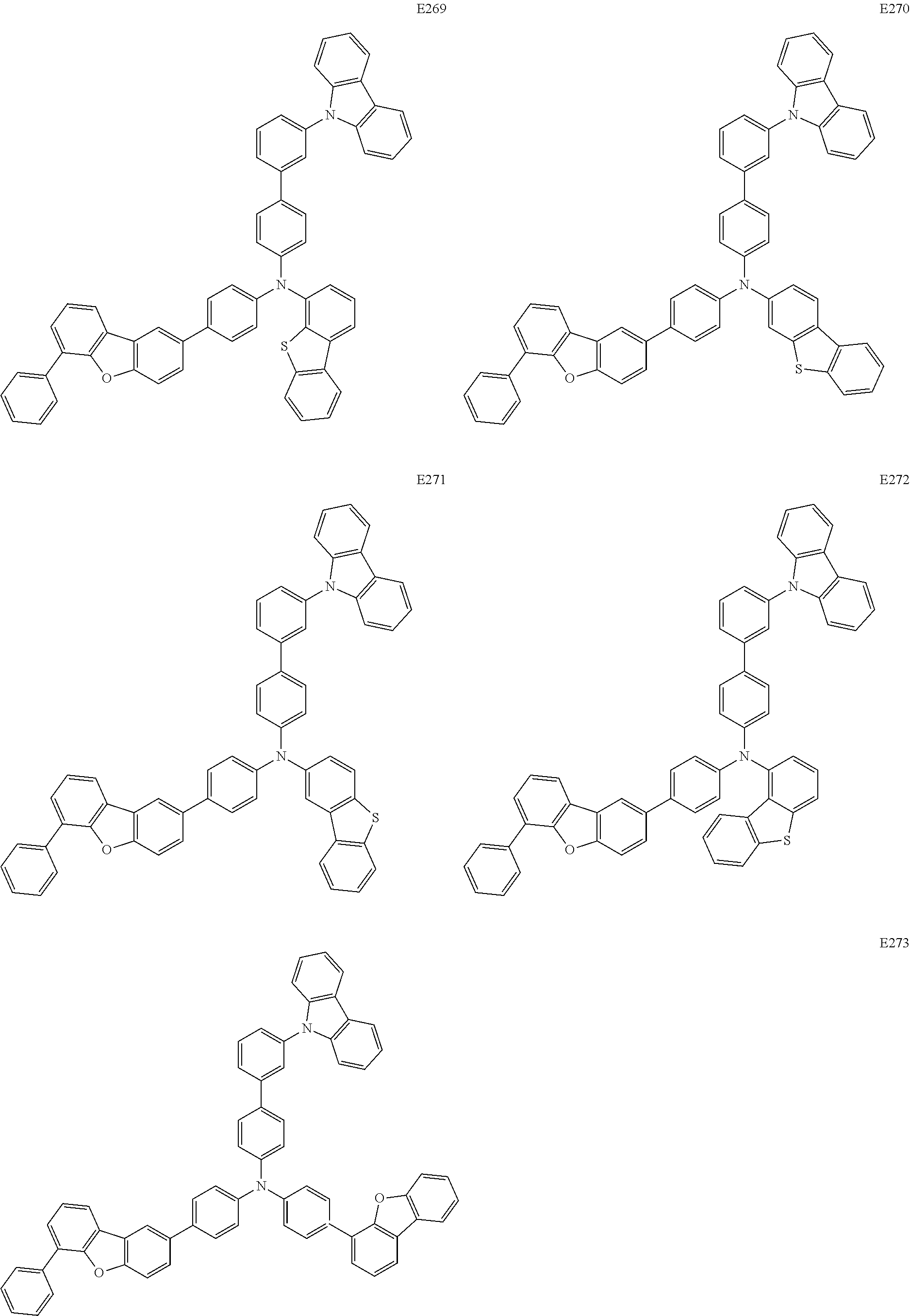 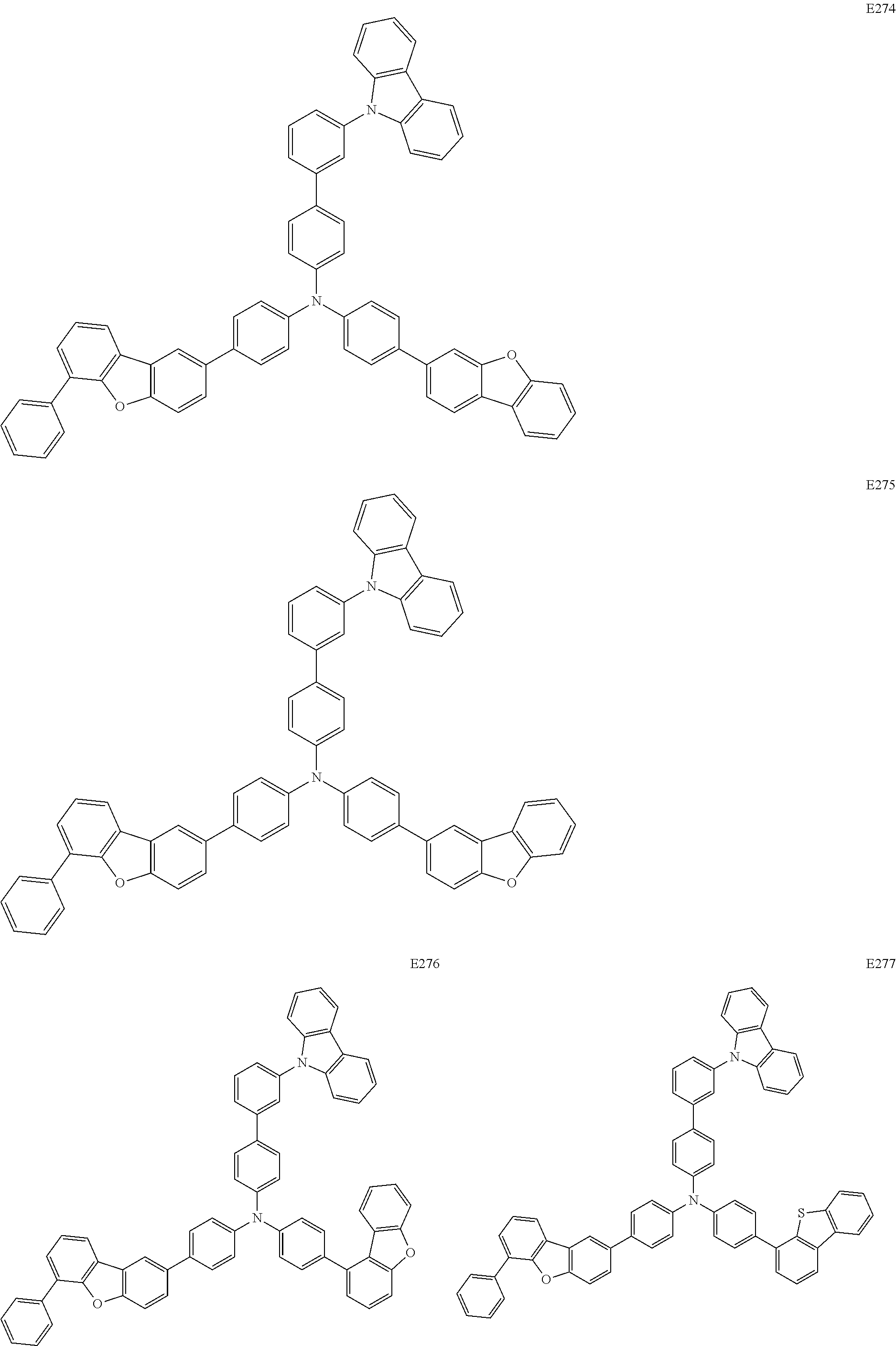 [SMILES: c1ccc(-c2cccc3c2oc2ccc(-c4ccc(N(c5ccc(-c6cccc(-n7c8ccccc8c8ccccc87)c6)cc5)c5ccc(-c6ccc7c(c6)oc6ccccc67)cc5)cc4)cc23)cc1 ; c1ccc(-c2cccc3c2oc2ccc(-c4ccc(N(c5ccc(-c6cccc(-n7c8ccccc8c8ccccc87)c6)cc5)c5ccc(-c6ccc7oc8ccccc8c7c6)cc5)cc4)cc23)cc1 ; c1ccc(-c2cccc3c2oc2ccc(-c4ccc(N(c5ccc(-c6cccc(-n7c8ccccc8c8ccccc87)c6)cc5)c5ccc(-c6cccc7oc8ccccc8c67)cc5)cc4)cc23)cc1 ; c1ccc(-c2cccc3c2oc2ccc(-c4ccc(N(c5ccc(-c6cccc(-n7c8ccccc8c8ccccc87)c6)cc5)c5ccc(-c6cccc7c6sc6ccccc67)cc5)cc4)cc23)cc1] 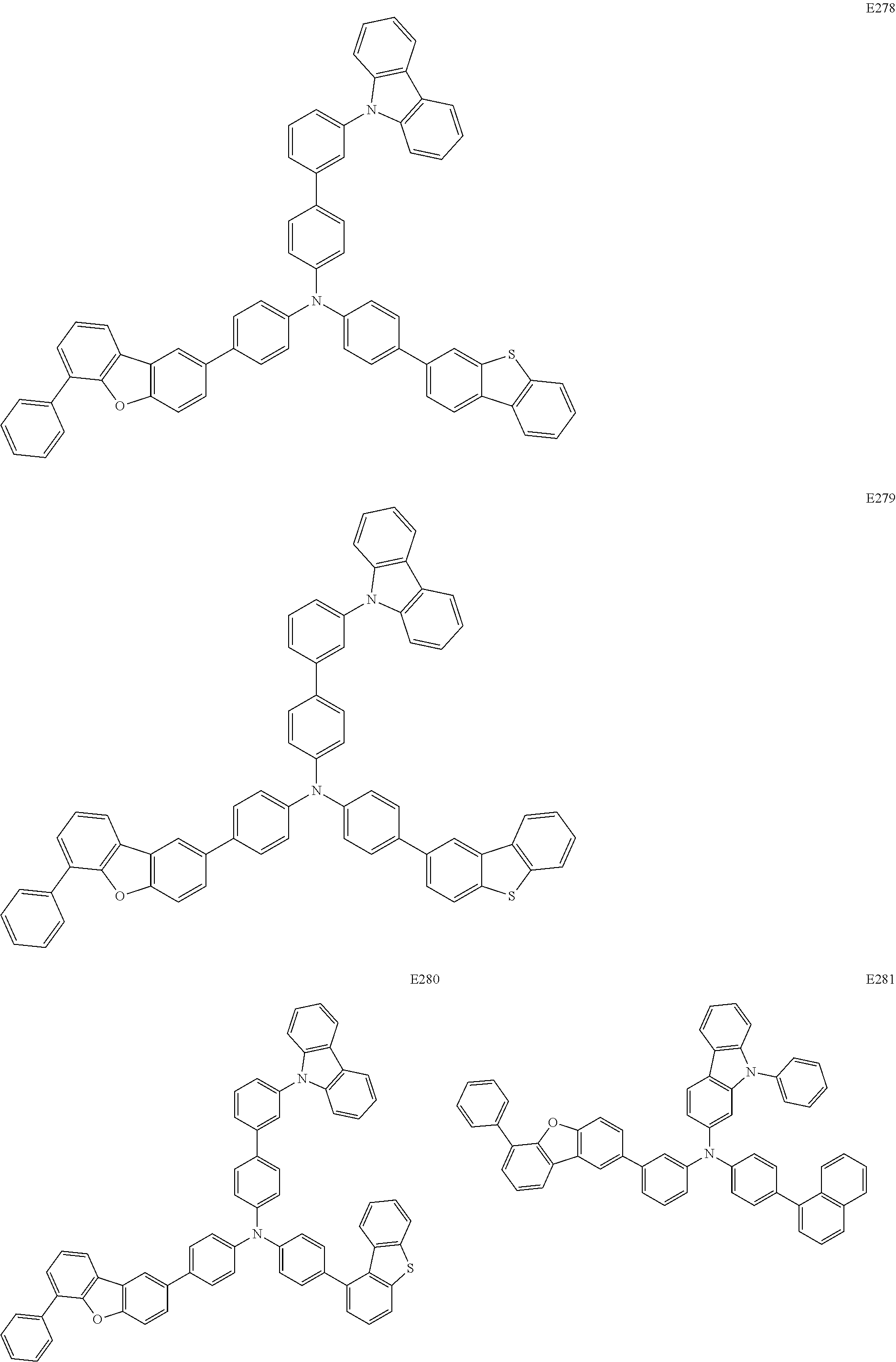 [SMILES: c1ccc(-c2cccc3c2oc2ccc(-c4ccc(N(c5ccc(-c6cccc(-n7c8ccccc8c8ccccc87)c6)cc5)c5ccc(-c6ccc7c(c6)sc6ccccc67)cc5)cc4)cc23)cc1 ; c1ccc(-c2cccc3c2oc2ccc(-c4ccc(N(c5ccc(-c6cccc(-n7c8ccccc8c8ccccc87)c6)cc5)c5ccc(-c6ccc7sc8ccccc8c7c6)cc5)cc4)cc23)cc1 ; c1ccc(-c2cccc3c2oc2ccc(-c4ccc(N(c5ccc(-c6cccc(-n7c8ccccc8c8ccccc87)c6)cc5)c5ccc(-c6cccc7sc8ccccc8c67)cc5)cc4)cc23)cc1 ; c1ccc(-c2cccc3c2oc2ccc(-c4cccc(N(c5ccc(-c6cccc7ccccc67)cc5)c5ccc6c7ccccc7n(-c7ccccc7)c6c5)c4)cc23)cc1] 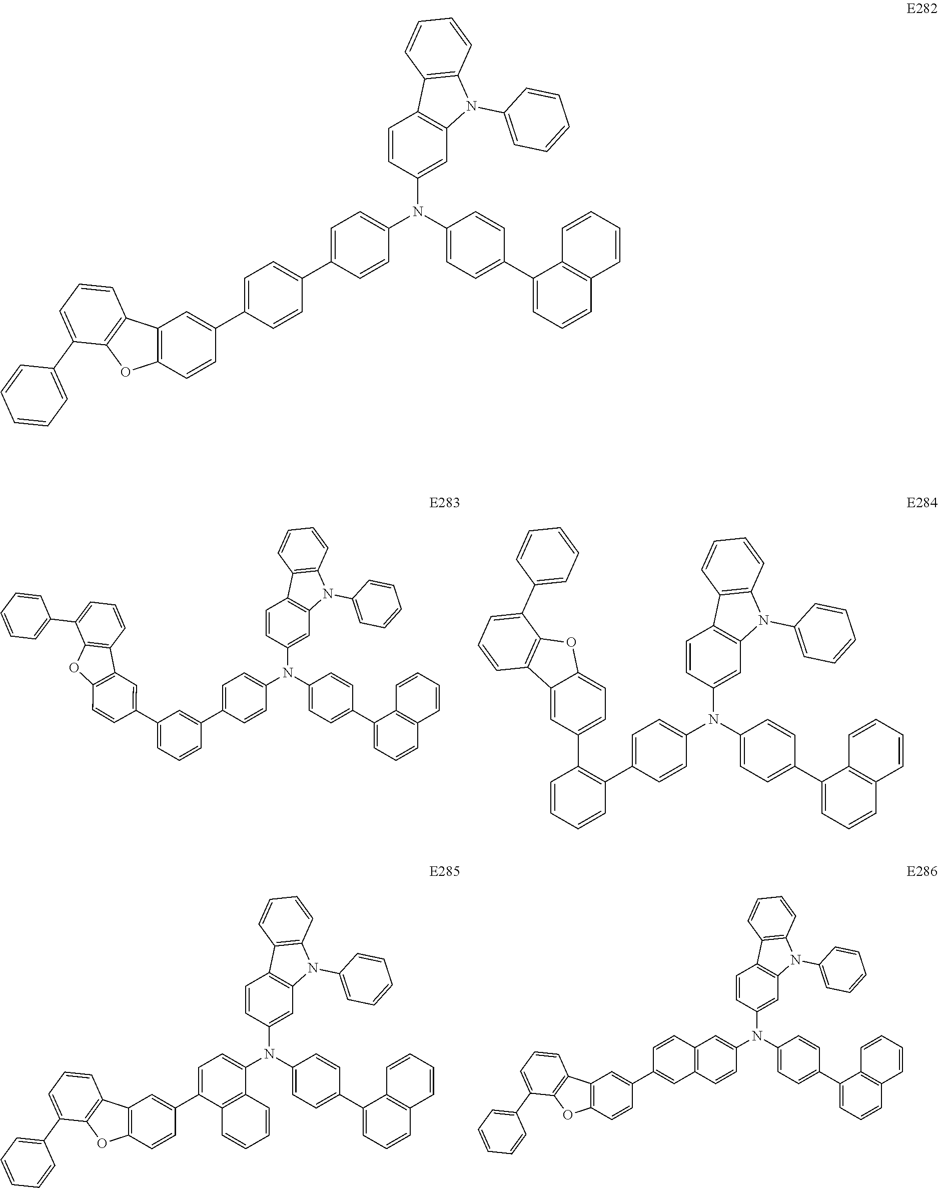 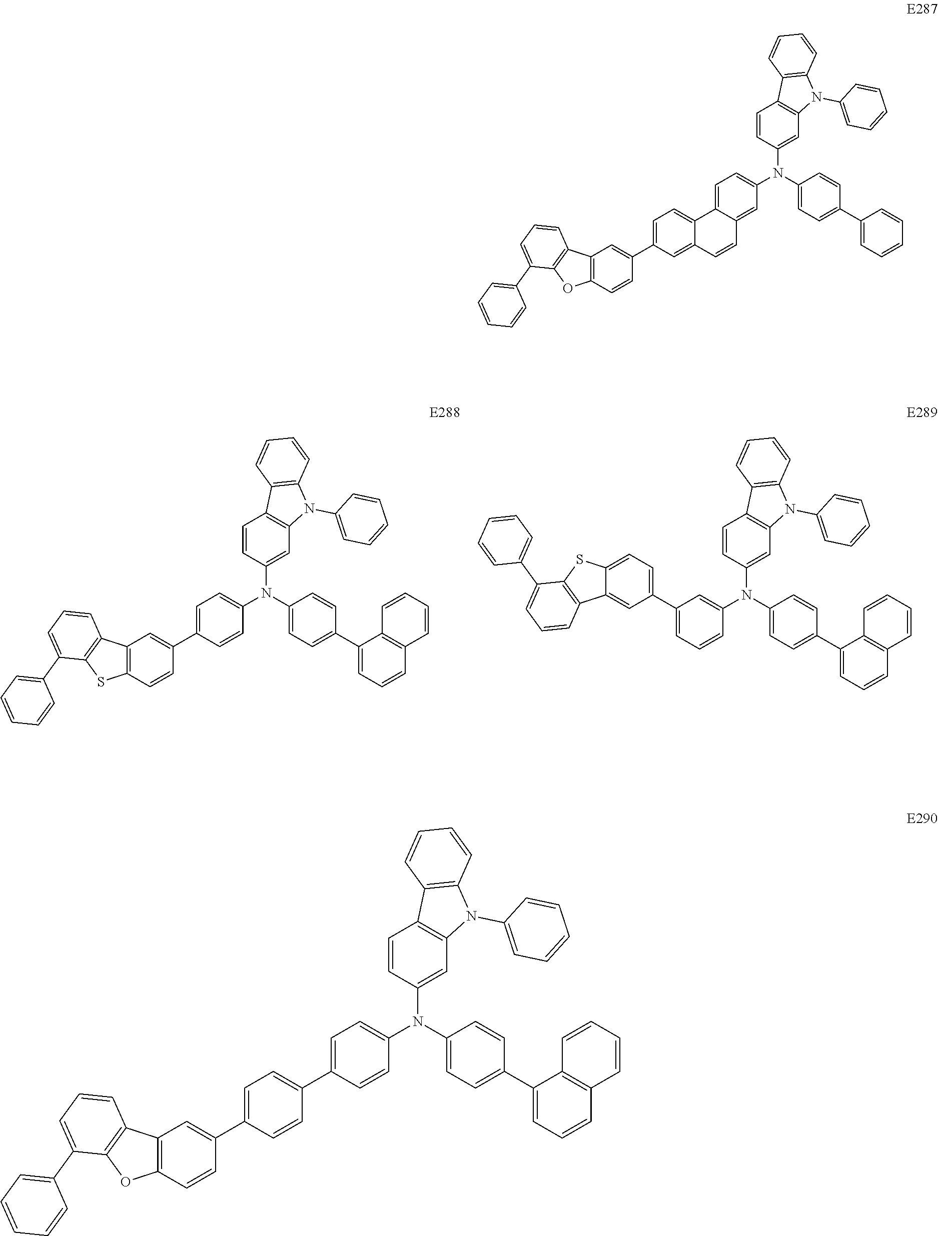 [SMILES: c1ccc(-c2ccc(N(c3ccc4c(ccc5cc(-c6ccc7oc8c(-c9ccccc9)cccc8c7c6)ccc54)c3)c3ccc4c5ccccc5n(-c5ccccc5)c4c3)cc2)cc1 ; c1ccc(-c2cccc3c2sc2ccc(-c4ccc(N(c5ccc(-c6cccc7ccccc67)cc5)c5ccc6c7ccccc7n(-c7ccccc7)c6c5)cc4)cc23)cc1 ; c1ccc(-c2cccc3c2sc2ccc(-c4cccc(N(c5ccc(-c6cccc7ccccc67)cc5)c5ccc6c7ccccc7n(-c7ccccc7)c6c5)c4)cc23)cc1 ; c1ccc(-c2cccc3c2oc2ccc(-c4ccc(-c5ccc(N(c6ccc(-c7cccc8ccccc78)cc6)c6ccc7c8ccccc8n(-c8ccccc8)c7c6)cc5)cc4)cc23)cc1]
